(19) United States
(12) Patent Application Publication
(10) Pub. No.: US 2025/0136569 A1
(43) Pub. Date: May 1, 2025

(54) COMPOUND, MATERIAL FOR ORGANIC ELECTROLUMINESCENT ELEMENT, ORGANIC ELECTROLUMINESCENT ELEMENT, AND ELECTRONIC DEVICE

(71) Applicant: IDEMITSU KOSAN CO.,LTD., Tokyo (JP)

(72) Inventors: Yusuke TAKAHASHI, Chiyoda-ku, Tokyo (JP); Hiroaki ITOI, Chiyoda-ku, Tokyo (JP); Tsukasa SAWATO, Chiyoda-ku, Tokyo (JP); Shota TANAKA, Chiyoda-ku, Tokyo (JP); Takuto FUKAMI, Chiyoda-ku, Tokyo (JP); Yongguk LEE, Chiyoda-ku, Tokyo (JP); Tasuku HAKETA, Chiyoda-ku, Tokyo (JP)

(73) Assignee: IDEMITSU KOSAN CO.,LTD., Tokyo (JP)

(21) Appl. No.: 18/718,576

(22) PCT Filed: Dec. 23, 2022
(86) PCT No.: PCT/JP2022/047680

§ 371 Date: Jun. 11, 2024

(30) Foreign Application Priority Data
Dec. 24, 2021 (JP) 2021-211424
Apr. 28, 2022 (JP) 2022-075172

(51) Int. Cl. C07D 311/92; C07B 59/00; C07D 335/08; C07D 405/04; C07D 407/04; C07D 409/04; H10K 50/15; H10K 85/20; H10K 85/60; H10K 101/00; H10K 101/10; H10K 101/30

(52) U.S. Cl. CPC C07D 311/92; C07B 59/004; C07D 335/08; C07D 405/04; C07D 407/04; C07D 409/04; H10K 50/15; H10K 85/20; H10K 85/615; H10K 85/653; H10K 85/655; H10K 85/6572; C07B 2200/05; H10K 2101/10; H10K 2101/27; H10K 2101/30

(57) ABSTRACT

To provide a compound for further improving the performance of an organic EL device, a material for an organic electroluminescent device, an organic electroluminescent device having further improved device capability, and an electronic device including such an organic electroluminescent device, and the compound is represented by formula (1). (Each symbol in the formula is as defined in the description.) In addition, the organic electroluminescent device contains the compound or has a highest occupied molecular orbital energy level HOMO, a triplet energy T1, and an 80% attenuation time t of a photoluminescence intensity PL within a predetermined range.

### TECHNICAL FIELD

The present invention relates to a compound, a material for organic electroluminescent devices, an organic electroluminescent device, and an electronic device including the organic electroluminescent device.

### BACKGROUND ART

In general, an organic electroluminescent device (which may be hereinafter referred to as an “organic EL device”) is constituted by an anode, a cathode, and an organic layer intervening between the anode and the cathode. When a voltage is applied between both the electrodes, electrons from the cathode side and holes from the anode side are injected into a light emitting region, and the injected electrons and holes are recombined in the light emitting region to generate an excited state, which then returns to the ground state to emit light. Accordingly, development of a material that efficiently transports electrons or holes into the light emitting region, and promotes recombination of the electrons and holes is important for providing a high-performance organic EL device.

PTLs 1 to 9 describe compounds used for a material for organic electroluminescent devices.

### CITATION LIST

### Patent Literature

PTL 1: JP2015-502960A
PTL 2: WO2018/168674
PTL 3: WO2020/004235
PTL 4: WO2020/106098
PTL 5: KR 10-2020-0077860A
PTL 6: JP2017-022194A
PTL 7: JP2020-169166A
PTL 8: WO2019/078701
PTL 9: US2015/001479A
PTL 10: WO2021/025162
PTL 11: WO2021/090933
PTL 12: U.S. Pat. No. 10,573,825B
PTL 13: US2015/069344A
PTL 14: WO2015/122711
PTL 15: CN111960954A
PTL 16: U.S. Pat. No. 10,600,969B
PTL 17: WO2021/210305

### SUMMARY OF INVENTION

### Technical Problem

Various compounds for organic EL devices have been reported, but a compound that further enhances the capability of an organic EL device has been still demanded.

The present invention has been made to solve the above-mentioned problems, and an object of the present invention is to provide a compound and a material for an organic electroluminescent device which further improve the capability of an organic EL device, an organic EL device having further improved device capability, and an electronic device including such an organic EL device.

### Solution to Problem

As a result of intensive research on the capability of an organic EL device containing a novel compound, the present inventors have found that the capability of an organic EL device containing a compound represented by the following formula (1) is further improved. In addition, the present inventors have found that the capability of an organic EL device containing a compound having specific physical properties is further improved.

In one embodiment, the present invention provides a compound represented by the following formula (1):

Ar¹—Ar² (1)
in which in the formula (1),
Ar¹ is represented by the following formula (1Aa), (1Ab), or (1B),

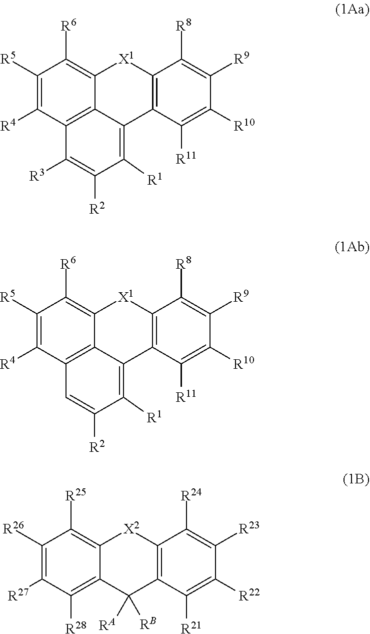

in which in the formulae (1A) and (1B),
X¹ is an oxygen atom or a sulfur atom,
R¹ to R⁶ and R⁸ to R¹¹ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
one selected from R¹ to R⁶ and R⁸ to R¹¹ which is not a hydrogen atom is a single bond bonded to Ar² or a group bonded to Ar², and
a pair of groups adjacent to each other among R¹ to R⁶ and R⁸ to R¹¹ which are not a hydrogen atom and which are not a single bond are bonded to each other to form a substituted or unsubstituted monocyclic ring, are bonded to each other to form a substituted or unsubstituted condensed ring, or are not bonded to each other and do not form a ring,
in which in the formula (1B),
X² is an oxygen atom or a sulfur atom,
R²¹ to R²⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R^{A} and R^{B} are each independently a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one selected from R²¹ to R²⁸, R^{A}, and R^{B} is a single bond bonded to Ar² or a group bonded to Ar²,
a pair of groups adjacent to each other among R²¹ to R²⁴ and R²⁵ to R²⁸ which are not a single bond are bonded to each other to form a substituted or unsubstituted monocyclic ring, are bonded to each other to form a substituted or unsubstituted condensed ring, or are not bonded to each other and do not form a ring, and
R^{A} and R^{B} are bonded to each other to form a substituted or unsubstituted monocyclic ring, are bonded to each other to form a substituted or unsubstituted condensed ring, or are not bonded to each other and do not form a ring,
Ar² is represented by the following formula (2A), (2B), (2C), (2D), (2E), or (2F),
provided that when Ar¹ is represented by the formula (1Aa), Ar² is represented by the following formula (2C), (2D), (2E), or (2F); when Ar¹ is represented by the formula (1Ab), Ar² is represented by the following formula (2A) or (2B); and when Ar¹ is represented by the formula (1B), Ar² is represented by the following formula (2A), (2B), (2C), (2D), (2E), or (2F),

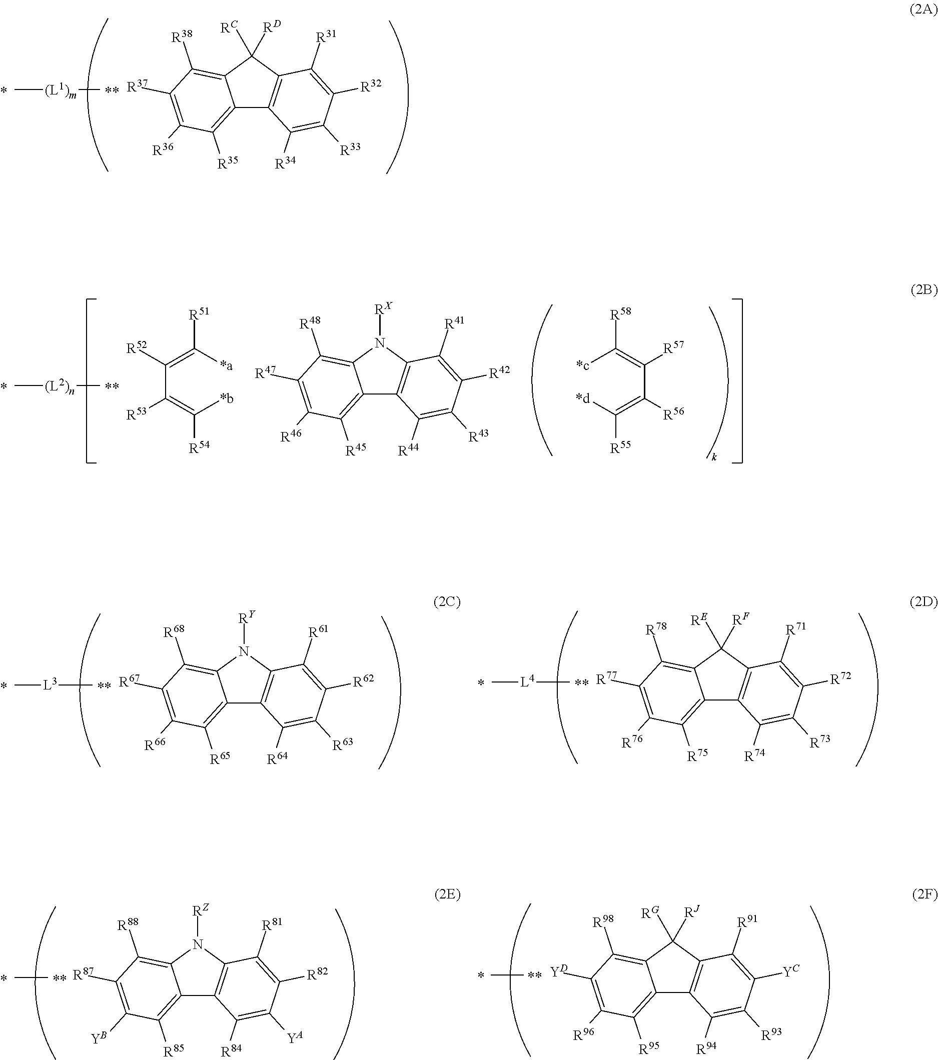

in which in the formula (2A),
L¹ is a substituted or unsubstituted arylene group having 6 to 30 ring carbon atoms, or a substituted or unsubstituted divalent heterocyclic group having 5 to 30 ring atoms,
* represents one end of L¹ or a single bond bonded to Ar¹, and ** represents the other end of L¹ or the single bond,
m is 0 or 1,
R³¹ to R³⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R^{C} and R^{D} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one selected from R³¹ to R³⁸, R^{C}, and R^{D} is a single bond bonded to ** or a group bonded to **,
a pair of groups adjacent to each other among R³¹ to R³⁸ which are not a single bond are bonded to each other to form a substituted or unsubstituted monocyclic ring, are bonded to each other to form a substituted or unsubstituted condensed ring, or are not bonded to each other and do not form a ring, and
R^{C} and R^{D} which are not a single bond are bonded to each other to form a substituted or unsubstituted monocyclic ring, are bonded to each other to form a substituted or unsubstituted condensed ring, or are not bonded to each other and do not form a ring,
in which in the formula (2B),
L² is a substituted or unsubstituted arylene group having 6 to 30 ring carbon atoms, or a substituted or unsubstituted divalent heterocyclic group having 5 to 30 ring atoms,
* represents one end of L² or a single bond bonded to Ar¹, and ** represents the other end of L² or the single bond,
n is 0 or 1,
R⁴¹ to R⁴⁸, R⁵¹ to R⁵⁴, and R⁵⁵ to R⁵⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
one of R⁴⁵ and R⁴⁶, R⁴⁶ and R⁴⁷, or R⁴⁷ and R⁴⁸ is a single bond bonded to *a, and the other is a single bond bonded to *b,
k is 0 or 1, when k is 1, one of R⁴¹ and R⁴², R⁴² and R⁴³, or R⁴³ and R⁴⁴ is a single bond bonded to *c, and the other is a single bond bonded to *d,
R^{X} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one selected from R⁴¹ to R⁴⁴ which are not bonded to *c and *d, R⁴⁵ to R⁴⁸ which are not bonded to *a and *b, R⁵¹ to R⁵⁴, R⁵⁵ to R⁵⁸, and R^{X} is a single bond bonded to ** or a group bonded to **, and
a pair of groups adjacent to each other among R⁴¹ to R⁴⁴ which are not bonded to *c and *d and are not a single bond bonded to the above **, R⁴⁵ to R⁴⁸ which are not bonded to *a and *b and are not a single bond bonded to the above **, R⁵¹ to R⁵⁴, and R⁵⁵ to R⁵⁸ are bonded to each other to form a substituted or unsubstituted monocyclic ring, are bonded to each other to form a substituted or unsubstituted condensed ring, or are not bonded to each other and do not form a ring,
in which in the formula (2C),
L³ is a substituted or unsubstituted arylene group having 6 to 30 ring carbon atoms, or a substituted or unsubstituted divalent heterocyclic group having 5 to 30 ring atoms,
* represents one end of L³ bonded to Ar¹, and ** represents the other end of L³,
R⁶¹ to R⁶⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R⁶¹ to R⁶⁸ are not bonded to each other and do not form a ring,
R^{Y} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and
one selected from R⁶¹ to R⁶⁸ and R^{Y} is a single bond bonded to ** or a group bonded to **,
in which in the formula (2D),
L⁴ is a substituted or unsubstituted arylene group having 6 to 30 ring carbon atoms, or a substituted or unsubstituted divalent heterocyclic group having 5 to 30 ring atoms,
* represents one end of L⁴ bonded to Ar¹, and ** represents the other end of L⁴,
R⁷¹ to R⁷⁸ are each independently a hydrogen atom, a substituted or unsubstituted alkyl group having 1 to 6 carbon atoms, an aryl group having 6 to 12 ring carbon atoms, or a heteroaryl group having 5 to 13 ring atoms,
R^{E} and R^{F} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one selected from R⁷¹ to R⁷⁸, R^{E}, and R^{F} is a single bond bonded to ** or a group bonded to **,
a pair of groups adjacent to each other among R⁷¹ to R⁷⁸ which are not a single bond are bonded to each other to form a substituted or unsubstituted monocyclic ring, are bonded to each other to form a substituted or unsubstituted condensed ring, or are not bonded to each other and do not form a ring, and
R^{E} and R^{F} which are not a single bond are bonded to each other to form a substituted or unsubstituted monocyclic ring, are bonded to each other to form a substituted or unsubstituted condensed ring, or are not bonded to each other and do not form a ring,
in which in the formula (2E),
* represents one end of a single bond bonded to Ar¹, and ** represents the other end of the single bond,
R⁸¹, R⁸², R⁸⁴, R⁸⁵, R⁸⁷, R⁸⁸, Y^{A}, and Y^{B} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R⁸¹, R⁸², R⁸⁴, R⁸⁵, R⁸⁷, R⁸⁸, Y^{A}, and Y^{B} are not bonded to each other and do not form a ring,
R^{Z} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and
one selected from R⁸¹, R⁸², R⁸⁴, R⁸⁵, R⁸⁷, R⁸⁸, and R^{Z} is a single bond bonded to ** or a group bonded to **,
in which in the formula (2F),
* represents one end of a single bond bonded to Ar¹, and ** represents the other end of the single bond,
R⁹¹, R⁹³ to R⁹⁶, R⁹⁸, Y^{C}, and Y^{D} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
one selected from R⁹¹, R⁹³ to R⁹⁶, R⁹⁸, R^{G}, and R^{J} is a single bond bonded to ** or a group bonded to **,
R^{G} and R^{J} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
a pair of groups adjacent to each other among R⁹¹, R⁹³ to R⁹⁶, R⁹⁸, Y^{C}, and Y^{D} which are not a single bond are bonded to each other to form a substituted or unsubstituted monocyclic ring, are bonded to each other to form a substituted or unsubstituted condensed ring, or are not bonded to each other and do not form a ring, and
R^{G} and R^{J} which are not a single bond are bonded to each other to form a substituted or unsubstituted monocyclic ring, are bonded to each other to form a substituted or unsubstituted condensed ring, or are not bonded to each other and do not form a ring.

In another embodiment, the present invention provides a material for an organic electroluminescent device containing the compound represented by the formula (1).

In still another embodiment, the present invention provides an organic electroluminescent device having a cathode, an anode, and organic layers intervening between the cathode and the anode, the organic layers including a light emitting layer, at least one layer of the organic layers containing the compound represented by the formula (1).

In still another embodiment, the present invention provides an organic electroluminescent device including an anode, a hole transporting zone, a light emitting layer, and a cathode in this order, in which the hole transporting zone contains a compound A satisfying the following conditions (A) to (C):
(A) a highest occupied molecular orbital energy level HOMO is −6.00 to −5.50 eV;
(B) a triplet energy T₁ is 2.10 eV or more; and
(C) an 80% attenuation time t of photoluminescence intensity PL is 0.10 h or more,
in which in the condition (C),
PL is an intensity of a photoluminescence emission spectrum when a measurement material in which a compound to be measured is formed into a film having a film thickness of 100 nm is irradiated with ultraviolet rays of 365 nm at an irradiation intensity I₁,
t is a time from the start of the irradiation with the ultraviolet rays until PL is attenuated to 80%,
I₁ is defined by the following mathematical expression (Equation 1),

I₁=I₀×(A₀/A₁) (Equation 1)
in which in the mathematical expression (Equation 1),
I₀ is an irradiation intensity at the time of PL measurement of a reference material in which a compound represented by the following chemical formula is formed into a film having a film thickness of 100 nm,

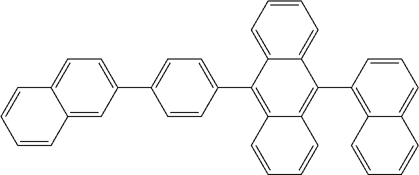 [SMILES: c1ccc2cc(-c3ccc(-c4c5ccccc5c(-c5cccc6ccccc56)c5ccccc45)cc3)ccc2c1]

A₀ is an absorptance of the reference material, and
A₁ is an absorptance of the measurement material,
each absorptance is defined by the following mathematical expression (Equation 2),

Absorptance=1−EXP(−4×3.1416×ko×d/w) (Equation 2)
in which in the mathematical expression (Equation 2),
ko is an extinction coefficient in an in-plane direction of a measurement material or a reference material on which a film of a compound to be measured is formed,
d is a film thickness of the measurement material or the reference material on which a film of the compound to be measured is formed, and
w is a wavelength of irradiation light.

In still another embodiment, the present invention provides an electronic device including the organic electroluminescent device.

### Advantageous Effects of Invention

The organic EL device containing the compound represented by the formula (1) shows an improved device capability. In addition, the organic EL device in which the compound A satisfying each condition described above is included in the hole transporting zone shows an improved device capability.

### BRIEF DESCRIPTION OF DRAWINGS

FIG. 1 is a schematic view showing an example of a layer structure of an organic EL device according to an embodiment of the present invention.

FIG. 2 is a schematic view showing another example of a layer structure of an organic EL device according to an embodiment of the present invention.

FIG. 3 is a schematic view showing still another example of a layer structure of an organic EL device according to an embodiment of the present invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained. Each of the substituents described in the description herein will be defined as described hereinafter, unless otherwise indicated in the description.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
a m-terphenyl-3′-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group, a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

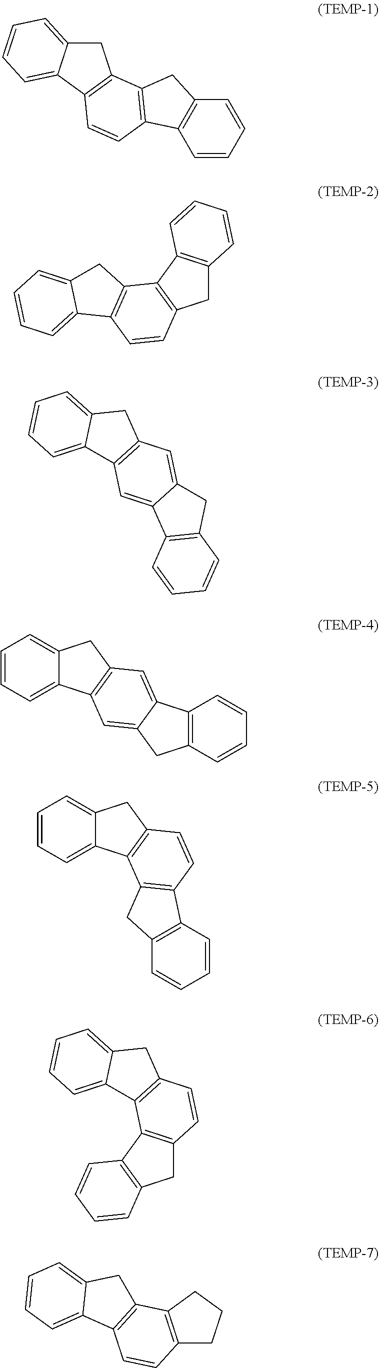

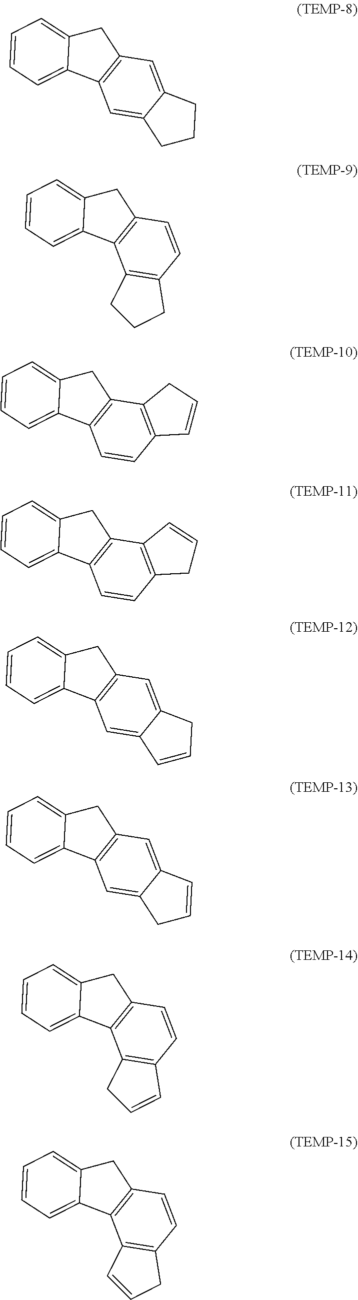

### Substituted Aryl Group (Set of Specific Examples G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom. In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

### Unsubstituted Heterocyclic Group Containing Nitrogen Atom (Set of Specific Examples G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group, a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and a diazacarbazolyl group.

### Unsubstituted Heterocyclic Group Containing Oxygen Atom (Set of Specific Examples G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing Sulfur Atom (Set of Specific Examples G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group, a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent Heterocyclic Group derived by removing One Hydrogen Atom from Ring Structures represented by General Formulae (TEMP-16) to (TEMP-33) (Set of Specific Examples G2A4)

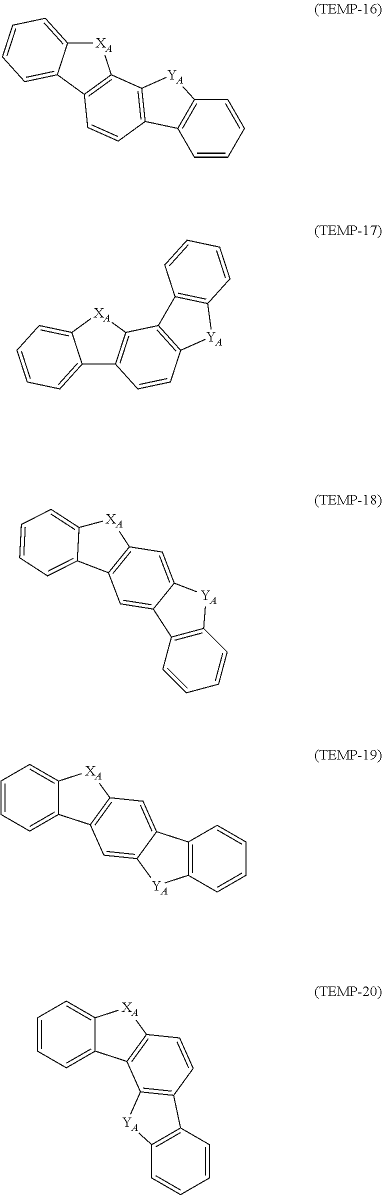

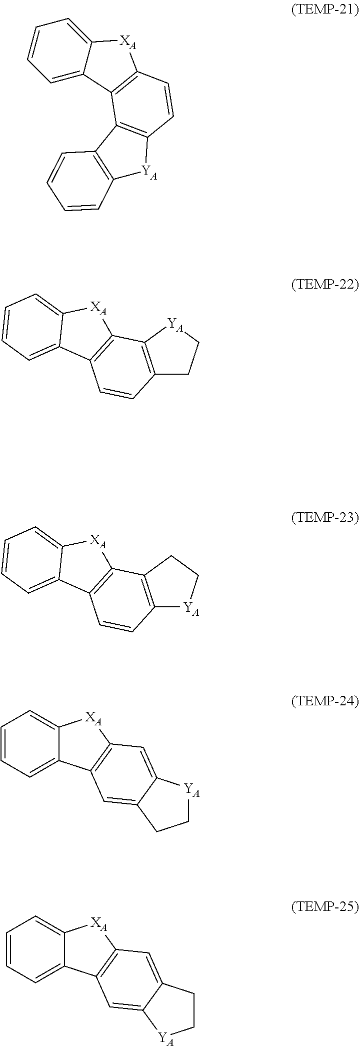

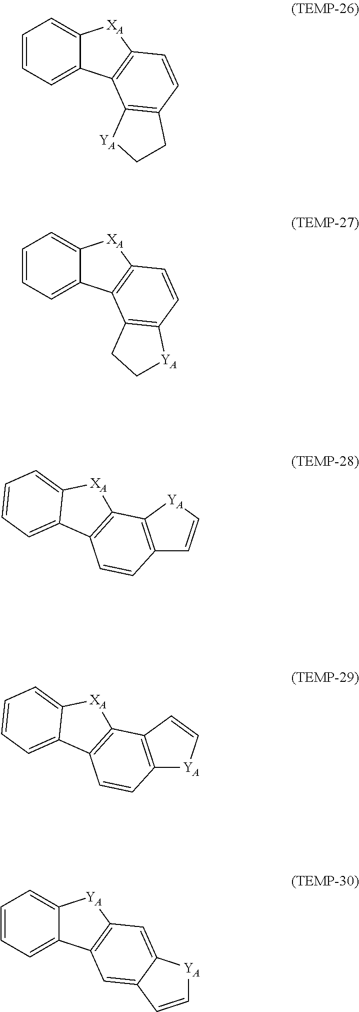

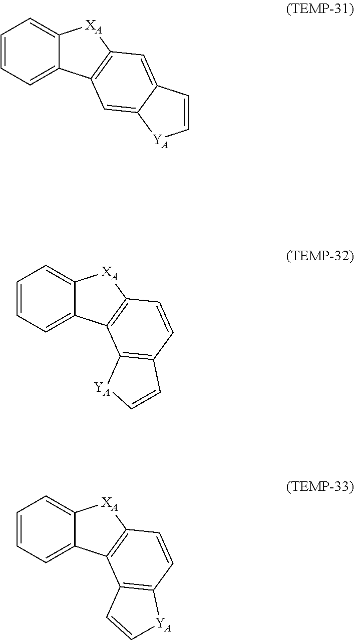

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

### Substituted Heterocyclic Group Containing Nitrogen Atom (Set of Specific Examples G2B1):

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and a biphenylquinazolinyl group.

### Substituted Heterocyclic Group Containing Oxygen Atom (Set of Specific Examples G2B2):

a phenyldibenzofuranyl group,
a methyldibenzofuranyl group,
a t-butyldibenzofuranyl group, and
a monovalent residual group of spiro[9H-xanthene-9,9′-[9H]fluorene].

### Substituted Heterocyclic Group Containing Sulfur Atom (Set of Specific Examples G2B3):

a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

Group formed by substituting one or more Hydrogen Atom of Monovalent Heterocyclic Group derived from Ring Structures represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A):

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B):

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and “the substituted alkenyl group” means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each has a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A):

a vinyl group,
an allyl group,
a 1-butenyl group,
a 2-butenyl group, and
a 3-butenyl group.

### Substituted Alkenyl Group (Set of Specific Examples G4B):

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

### Unsubstituted Alkynyl Group (Set of Specific Examples G5A):

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Set of Specific Examples G6B):

a 4-methylcyclohexyl group.
   Group Represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6). Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

### Group Represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

### Group Represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G2),
—N(G2)(G2),
—N(G1)(G2),
—N(G3)(G3), and
—N(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranly group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

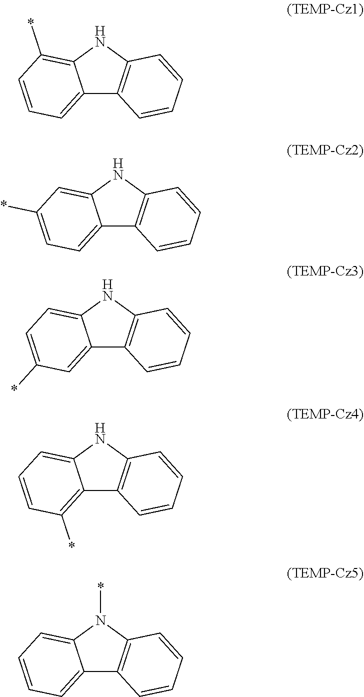 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

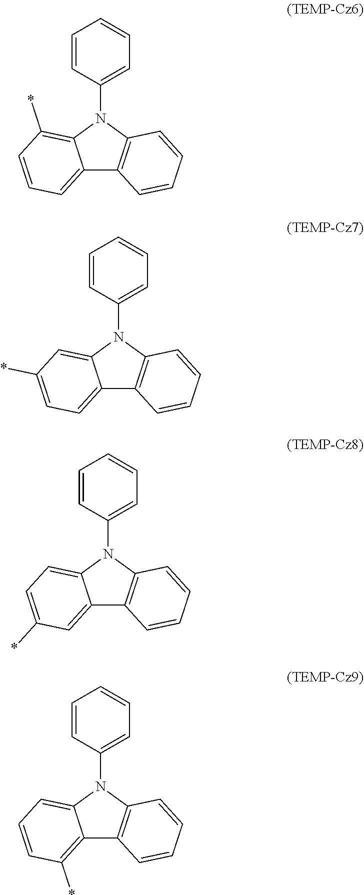 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

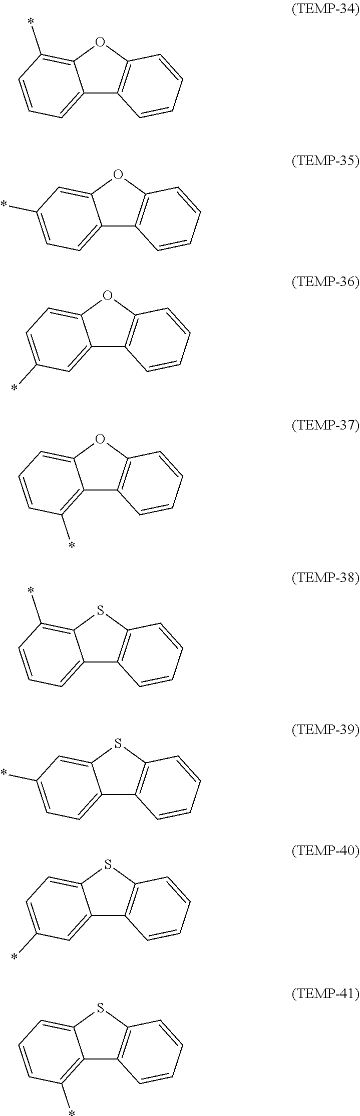

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

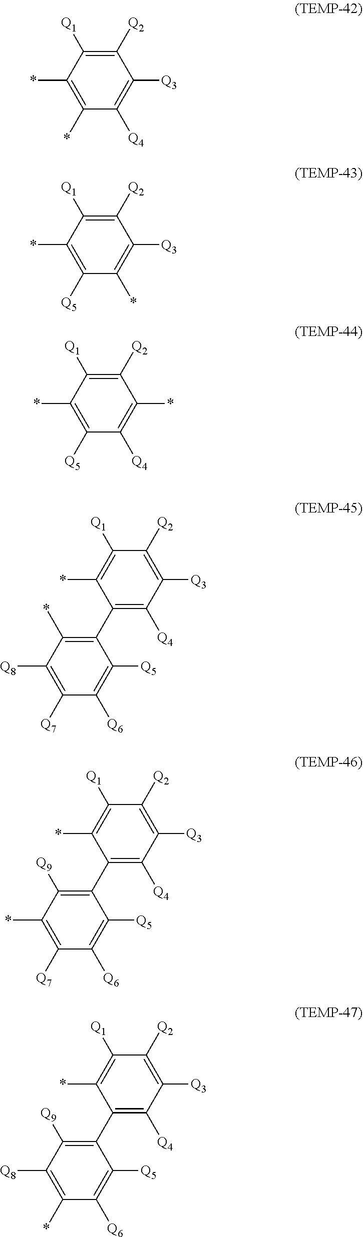

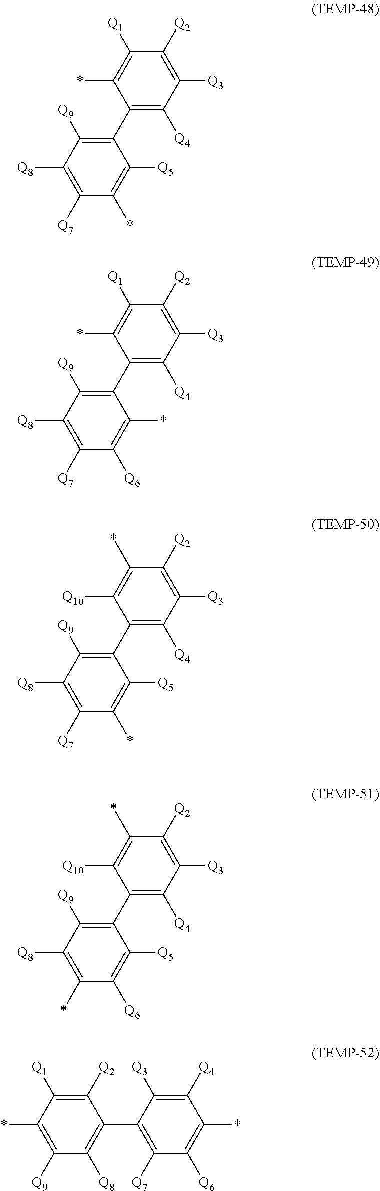

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

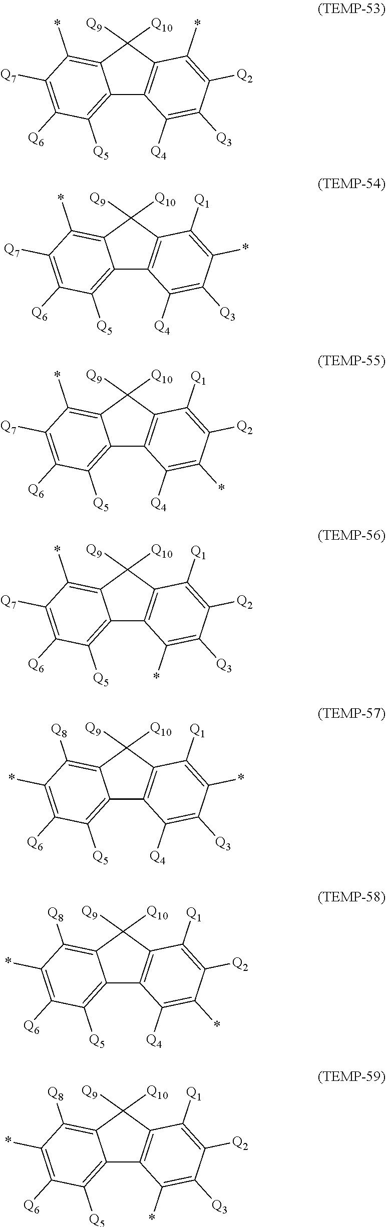

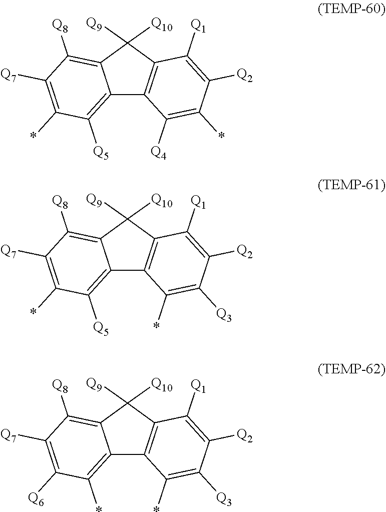

In the general formulae (TEMP-53) to (TEMP-62), Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

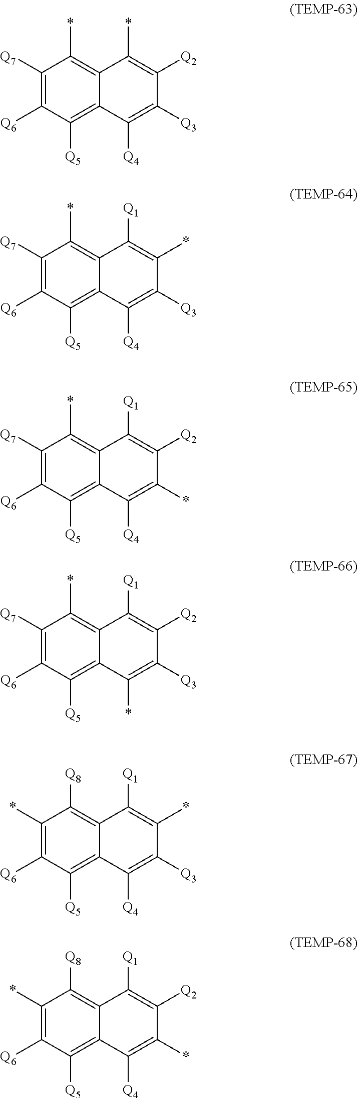

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

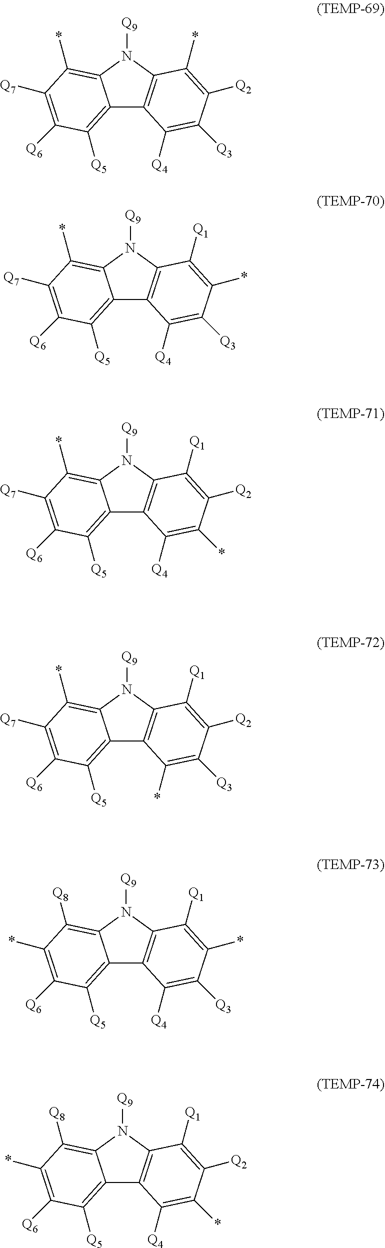

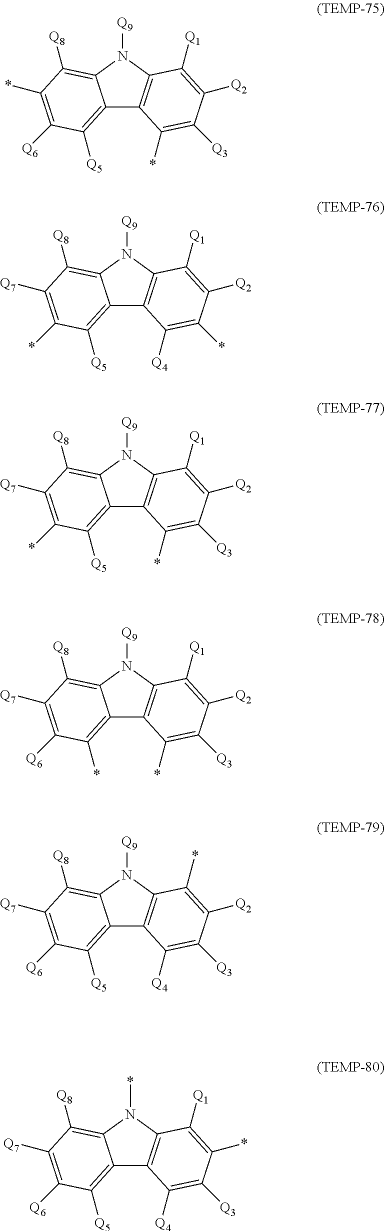

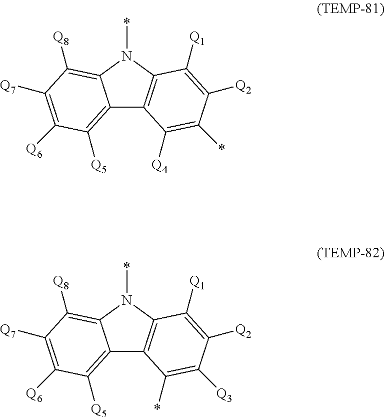

In the general formulae (TEMP-69) to (TEMP-82), Q₁ to Q₉ each independently represent a hydrogen atom or a substituent.

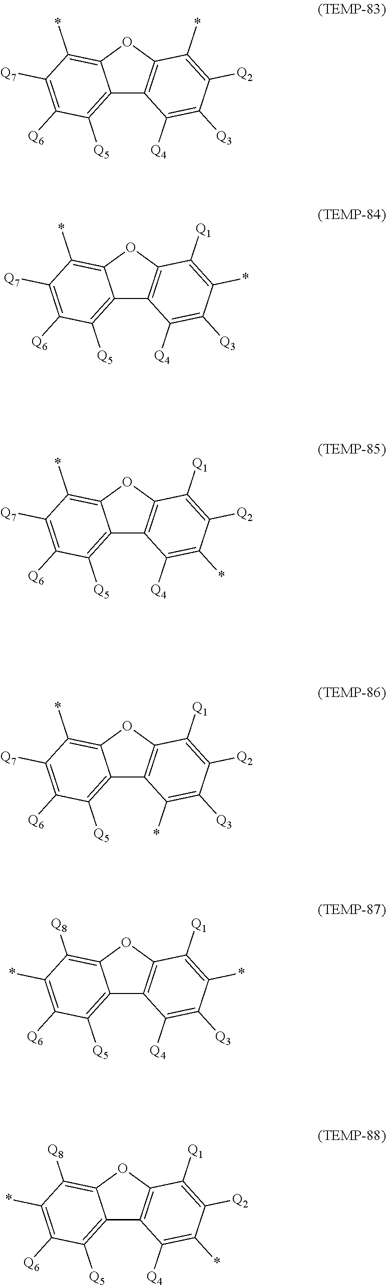

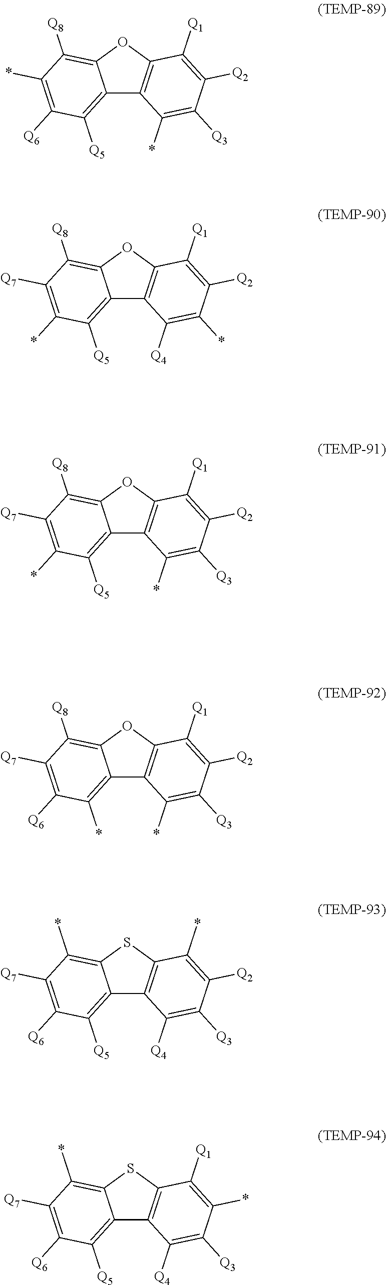

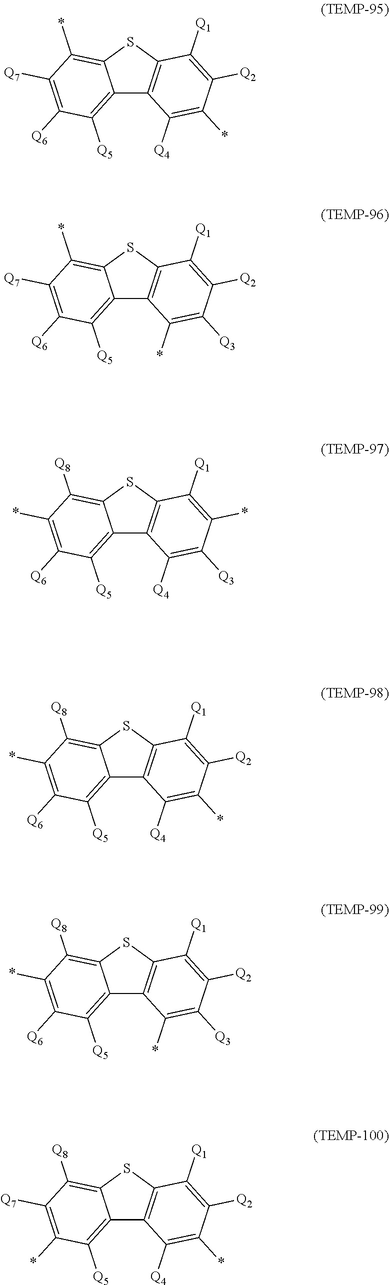

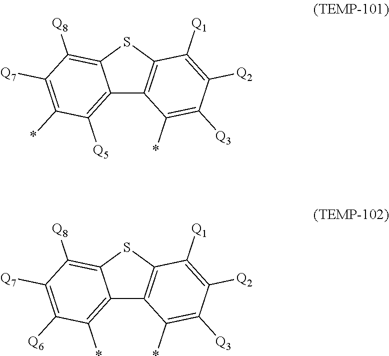

In the general formulae (TEMP-83) to (TEMP-102), Q₁ to Q₈ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

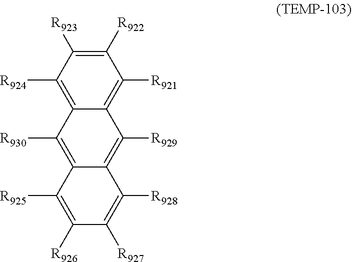

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

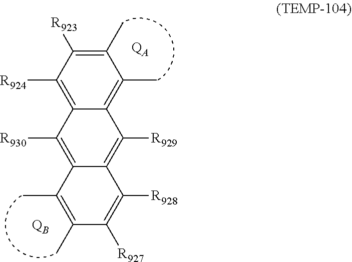

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Qc, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Qc share R₉₂₂.

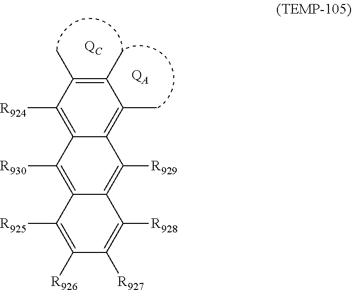

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Qc formed in the general formula (TEMP-105) each are a “condensed ring”. The ring Q_{A} and the ring Qc in the general formula (TEMP-105) form a condensed ring through condensation of the ring Q_{A} and the ring Qc. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or anon-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more each are bonded to each other to form a substituted or unsubstituted monocyclic ring”, and the case where “one or more combinations of combinations each including adjacent two or more each are bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

The compound of the present invention will be described below.

The compound according to one embodiment of the present invention is represented by the following formula (1).

However, hereinafter, the compound of the present invention represented by the formula (1) and each formula included in the formula (1) described later may be simply referred to as “compound (1)”, “inventive compound (1)”, or “inventive compound”.

Ar¹—Ar² (1)

Hereinafter, symbols in the formula (1) and each formula included in the formula (1) described later will be described. The same symbols have the same meanings.

In the formula (1), Ar¹ is represented by the following formula (1Aa), (1Ab), or (1B).

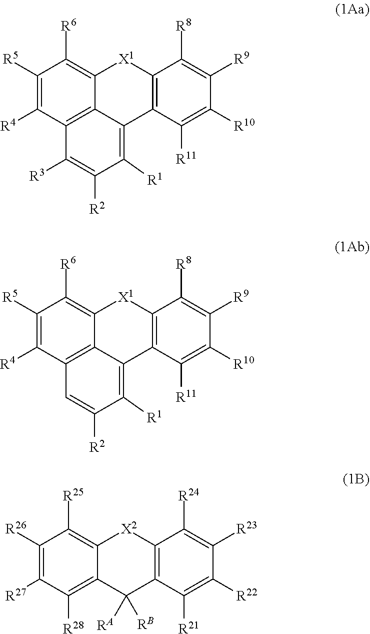

In the formulae (1Aa) and (1Ab), X¹ is preferably an oxygen atom or a sulfur atom, and more preferably an oxygen atom.

In the formulae (1Aa) and (1Ab), R¹ to R⁶ and R⁸ to R¹¹ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms, and preferably each independently a hydrogen atom, a substituted or unsubstituted alkyl group having 1 to 6 carbon atoms, or a substituted or unsubstituted aryl group having 6 to 12 ring carbon atoms.

In the formulae (1Aa) and (1Ab), one selected from R¹ to R⁶ and R⁸ to R¹¹ which is not a hydrogen atom is a single bond bonded to Ar² or a group bonded to Ar², R¹⁰ is preferably a single bond bonded to Ar² or a group bonded to Ar².

In the formulae (1Aa) and (1Ab), a pair of groups adjacent to each other among R¹ to R⁶ and R⁸ to R¹¹ which are not a hydrogen atom and which are not a single bond are bonded to each other to form a substituted or unsubstituted monocyclic ring, are bonded to each other to form a substituted or unsubstituted condensed ring, or are not bonded to each other and do not form a ring.

The unsubstituted monocyclic ring formed by R¹ to R⁶ and R⁸ to R¹¹ which are not a hydrogen atom and which are not a single bond is preferably a monocyclic ring having 3 or more and 6 or less ring atoms, for example, a benzene ring, a furan ring, or a thiophene ring, and preferably a benzene ring.

In the formula (1), X² is preferably an oxygen atom or a sulfur atom, and more preferably an oxygen atom.

In the formula (1), R²¹ to R²⁸ are each independently a hydrogen atom, a substituted or unsubstituted alkyl group having 1 to 6 carbon atoms, a substituted or unsubstituted aryl group having 6 to 12 ring carbon atoms, or a heteroaryl group having 5 to 13 ring atoms.

In the formula (1B), R^{A} and R^{B} are each independently a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and preferably a substituted or unsubstituted alkyl group having 1 to 30 carbon atoms or a substituted or unsubstituted aryl group having 6 to 30 ring carbon atoms.

In the formula (1B), one selected from R²¹ to R²⁸, R^{A}, and R^{B} is a single bond bonded to Ar² or a group bonded to Ar².

In the formula (1), a pair of groups adjacent to each other among R²¹ to R²⁴ and R²⁵ to R²⁸ which are not a single bond are bonded to each other to form a substituted or unsubstituted monocyclic ring, are bonded to each other to form a substituted or unsubstituted condensed ring, or are not bonded to each other and do not form a ring.

The unsubstituted monocyclic ring formed by R²¹ to R²⁴ and R²⁵ to R²⁸ which are not a hydrogen atom and which are not a single bond is preferably a monocyclic ring having 3 or more and 6 or less ring atoms, for example, a benzene ring, a furan ring, or a thiophene ring, and preferably a benzene ring.

In the formula (1), R^{A} and R^{B} which are not a single bond are bonded to each other to form a substituted or unsubstituted monocyclic ring, are bonded to each other to form a substituted or unsubstituted condensed ring, or are not bonded to each other and do not form a ring.

The unsubstituted alkyl group represented by R¹ to R⁶, R⁸ to R¹¹, and R²¹ to R²⁸ is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R¹ to R⁶, R⁸ to R¹¹, and R²¹ to R²⁸ is preferably a phenyl group, a biphenyl group, or a naphthyl group, and more preferably a phenyl group.

The unsubstituted heterocyclic group represented by R¹ to R⁶, R⁸ to R¹¹, and R²¹ to R²⁸ is preferably a pyridyl group or a quinazolinyl group.

The unsubstituted monocyclic ring formed by R^{C} and R^{D} which are not a single bond is, for example, a benzene ring, a cyclopentane ring, or a cyclohexane ring.

The unsubstituted condensed ring formed by R^{C} and R^{D} which are not a single bond, the unsubstituted condensed ring formed by R^{E} and R^{F} which are not a single bond, and the unsubstituted condensed ring formed by R^{G} and R^{J} which are not a single bond are, for example, a naphthalene ring or an anthracene ring.

The unsubstituted alkyl group represented by R^{A} and R^{B} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{A} and R^{B} is preferably a phenyl group, a biphenyl group, a naphthyl group, a terphenyl group, a phenanthrenyl group, a triphenylenyl group, or a fluorenyl group, more preferably a phenyl group, a biphenyl group, a naphthyl group, or a phenanthrenyl group, still more preferably a phenyl group, a naphthyl group, or a phenanthrenyl group, and even more preferably a phenyl group or a naphthyl group.

The unsubstituted heterocyclic group represented by R^{A} and R^{B} is preferably a dibenzofuranyl group, a dibenzothiophenyl group, or a pyridyl group, and more preferably a dibenzofuranyl group or a dibenzothiophenyl group.

In the formula (1),
in the case where Ar¹ is represented by the formula (1Aa) or (1Ab), it is preferable that any one of R¹ to R¹¹ which is not a hydrogen atom is a single bond bonded to Ar², or a monocyclic ring or a condensed ring formed of a pair of groups adjacent to each other among R¹ to R¹¹ is bonded to Ar², and
in the case where Ar¹ is represented by the formula (1), it is preferable that any one of R²¹ to R²⁸, R^{A}, and R^{B} which is not a hydrogen atom is a single bond bonded to Ar², a monocyclic ring or a condensed ring formed of a pair of groups adjacent to each other among R²¹ to R²⁸ is bonded to Ar², or a monocyclic ring or a condensed ring formed of R^{A} and R^{B} is bonded to Ar².

In the formula (1), Ar² is represented by the following formula (2A), (2B), (2C), (2D), (2E), or (2F), provided that in the case where Ar¹ is represented by the formula (1Aa), Ar² is represented by the formula (2C), (2D), (2E), or (2F).

In the case where Ar¹ is represented by the formula (1Ab), Ar² is represented by the following formula (2A) or (2B). In the case where Ar¹ is represented by the formula (1), Ar² is represented by the following formula (2A), (2B), (2C), (2D), (2E), or (2F).

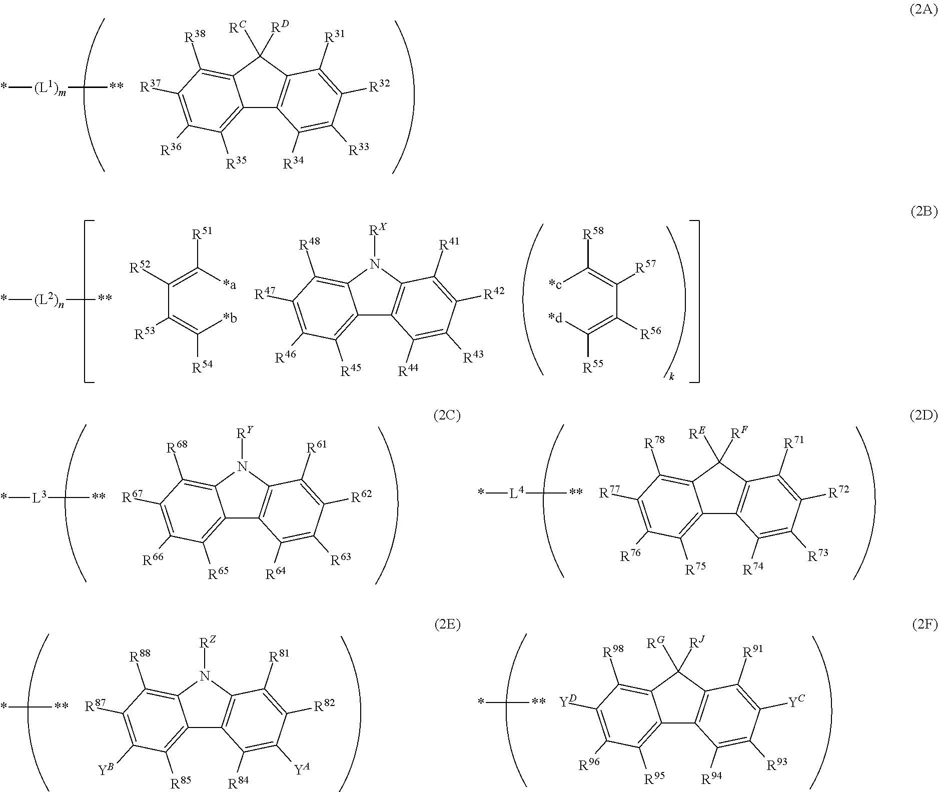

In the formula (2A), L¹ is a substituted or unsubstituted arylene group having 6 to 30 ring carbon atoms, or a substituted or unsubstituted divalent heterocyclic group having 5 to 30 ring atoms.

In the formula (2A), * represents one end of L¹ or a single bond bonded to Ar¹, and ** represents the other end of L¹ or the single bond.

In the formula (2A), m is 0 or 1.

In the formula (2A), R³¹ to R³⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2A), R^{C} and R^{D} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2A), one selected from R³¹ to R³⁸, R^{C}, and R^{D} is a single bond bonded to ** or a group bonded to **.

In the formula (2A), a pair of groups adjacent to each other among R³¹ to R³⁸ which are not a single bond are bonded to each other to form a substituted or unsubstituted monocyclic ring, are bonded to each other to form a substituted or unsubstituted condensed ring, or are not bonded to each other and do not form a ring.

In the formula (2A), R^{C} and R^{D} which are not a single bond are bonded to each other to form a substituted or unsubstituted monocyclic ring, are bonded to each other to form a substituted or unsubstituted condensed ring, or are not bonded to each other and do not form a ring.

In the formula (2B), L² is a substituted or unsubstituted arylene group having 6 to 30 ring carbon atoms, or a substituted or unsubstituted divalent heterocyclic group having 5 to 30 ring atoms.

In the formula (2B), * represents one end of L² or a single bond bonded to Ar¹, and ** represents the other end of L² or the single bond.

In the formula (2B), n is 0 or 1.

In the formula (2B), R⁴¹ to R⁴⁸, R⁵¹ to R⁵⁴, and R⁵⁵ to R⁵⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

One of R⁴⁵ and R⁴⁶, R⁴⁶ and R⁴⁷, or R⁴⁷ and R⁴⁸ is a single bond bonded to *a, and the other is a single bond bonded to *b.

In the formula (2B), k is 0 or 1.

When k is 1, one of R⁴¹ and R⁴², R⁴² and R⁴³, or R⁴³ and R⁴⁴ is a single bond bonded to *c, and the other is a single bond bonded to *d.

In the formula (2B), R^{X} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2B), one selected from R⁴¹ to R⁴⁴ which are not bonded to *c and *d, R⁴⁵ to R⁴⁸ which are not bonded to *a and *b, R⁵¹ to R⁵⁴, R⁵⁵ to R⁵⁸, and R^{X} is a single bond bonded to ** or a group bonded to **.

In the formula (2B), a pair of groups adjacent to each other among R⁴¹ to R⁴⁴ which are not bonded to *c and *d and are not a single bond bonded to the above **, R⁴⁵ to R⁴⁸ which are not bonded to *a and *b and are not a single bond bonded to the above **, R⁵¹ to R⁵⁴, and R⁵⁵ to R⁵⁸ are bonded to each other to form a substituted or unsubstituted monocyclic ring, are bonded to each other to form a substituted or unsubstituted condensed ring, or are not bonded to each other and do not form a ring.

In the formula (2C), L³ is a substituted or unsubstituted arylene group having 6 to 30 ring carbon atoms, or a substituted or unsubstituted divalent heterocyclic group having 5 to 30 ring atoms.

In the formula (2C), * represents one end of L³ bonded to Ar¹, and ** represents the other end of L³.

In the formula (2C), R⁶¹ to R⁶⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

R⁶¹ to R⁶⁸ are not bonded to each other and do not form a ring.

In the formula (2C), R^{Y} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2C), one selected from R⁶¹ to R⁶⁸ and R^{Y} is a single bond bonded to ** or a group bonded to **.

In the formula (2D), L⁴ is a substituted or unsubstituted arylene group having 6 to 30 ring carbon atoms, or a substituted or unsubstituted divalent heterocyclic group having 5 to 30 ring atoms.

In the formula (2D), * represents one end of L⁴ bonded to Ar¹, and ** represents the other end of L⁴.

In the formula (2D), R⁷¹ to R⁷⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2D), R^{E} and R^{F} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2D), one selected from R⁷¹ to R⁷⁸, R^{E}, and R^{F} is a single bond bonded to ** or a group bonded to **.

A pair of groups adjacent to each other among R⁷¹ to R⁷⁸ which are not a single bond are bonded to each other to form a substituted or unsubstituted monocyclic ring, are bonded to each other to form a substituted or unsubstituted condensed ring, or are not bonded to each other and do not form a ring.

R^{E} and R^{F} which are not a single bond are bonded to each other to form a substituted or unsubstituted monocyclic ring, are bonded to each other to form a substituted or unsubstituted condensed ring, or are not bonded to each other and do not form a ring.

In the formula (2E), * represents one end of a single bond bonded to Ar¹, and ** represents the other end of the single bond.

In the formula (2E), * represents one end of a single bond bonded to Ar¹, and ** represents the other end of the single bond.

In the formula (2E), R⁸¹, R⁸², R⁸⁴, R⁸⁵, R⁸⁷, R⁸⁸, Y^{A}, and Y^{B} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

R⁸¹, R⁸², R⁸⁴, R⁸⁵, R⁸⁷, R⁸⁸, Y^{A} and Y^{B} are not bonded to each other and do not form a ring.

In the formula (2E), R^{Z} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2E), one selected from R⁸¹, R⁸², R⁸⁴, R⁸⁵, R⁸⁷, R⁸⁸, and R^{Z} is a single bond bonded to ** or a group bonded to **.

In the formula (2F), * represents one end of a single bond bonded to Ar¹, and ** represents the other end of the single bond.

In the formula (2F), R⁹¹, R⁹³ to R⁹⁶, R⁹⁸, Y^{C}, and Y^{D} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2F), R^{G} and R^{J} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2F), one selected from R⁹¹, R⁹³ to R⁹⁶, R⁹⁸, R^{G}, and R^{J} is a single bond bonded to ** or a group bonded to **.

In the formula (2F), a pair of groups adjacent to each other among R⁹¹, R⁹³ to R⁹⁶, R⁹⁸, Y^{C}, and Y^{D} which are not a single bond are bonded to each other to form a substituted or unsubstituted monocyclic ring, are bonded to each other to form a substituted or unsubstituted condensed ring, or are not bonded to each other and do not form a ring.

In the formula (2F), R^{G} and R^{J} which are not a single bond are bonded to each other to form a substituted or unsubstituted monocyclic ring, are bonded to each other to form a substituted or unsubstituted condensed ring, or are not bonded to each other and do not form a ring.

The unsubstituted alkyl group represented by R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, Y^{A}, Y^{B}, Y^{C}, and Y^{D} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, Y^{A}, Y^{B}, Y^{C}, and Y^{D} is preferably a phenyl group, a biphenyl group, or a naphthyl group, and more preferably a phenyl group.

The unsubstituted heterocyclic group represented by R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R¹⁴, R⁸⁵, R⁸⁷, R⁸⁸, R⁹¹, R⁹², R⁹⁴, R⁹⁵, R⁹⁷, Y^{A}, Y^{B}, Y^{C} and Y^{D} is preferably a pyridyl group or a quinazolinyl group.

The unsubstituted alkyl group represented by R^{C}, R^{D}, R^{E}, R^{F}, R^{G}, and R is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{C}, R^{D}, R^{E}, R^{F}, R^{G}, and R^{J} is preferably a phenyl group, a biphenyl group, a naphthyl group, a terphenyl group, a phenanthrenyl group, a triphenylenyl group, or a fluorenyl group, more preferably a phenyl group, a biphenyl group, a naphthyl group, or a phenanthrenyl group, still more preferably a phenyl group, a naphthyl group, or a phenanthrenyl group, and even more preferably a phenyl group or a naphthyl group.

The unsubstituted heteroaryl group represented by R^{C}, R^{D}, R^{E}, R^{F}, R^{G}, and R is preferably a dibenzofuranyl group, a dibenzothiophenyl group, or a pyridyl group, and more preferably a dibenzofuranyl group or a dibenzothiophenyl group.

The unsubstituted alkyl group represented by R^{X}, R^{Y}, and R^{Z} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{X}, R^{Y}, and R^{Z} is preferably a phenyl group, a biphenyl group, a naphthyl group, a terphenyl group, a phenanthrenyl group, a triphenylenyl group, or a fluorenyl group, more preferably a phenyl group, a biphenyl group, a naphthyl group, or a phenanthrenyl group, still more preferably a phenyl group, a naphthyl group, or a phenanthrenyl group, and even more preferably a phenyl group or a naphthyl group.

The unsubstituted heteroaryl group represented by R^{X}, R^{Y}, and R^{Z} is preferably a dibenzofuranyl group, a dibenzothiophenyl group, or a pyridyl group, and more preferably a dibenzofuranyl group or a dibenzothiophenyl group.

The details of the substituted or unsubstituted arylene group having 6 to 30 ring carbon atoms represented by L¹ to L⁴ are as described above in the section of “Substituents in Description”. The substituted or unsubstituted arylene group represented by L¹ to L⁴ is preferably each independently a substituted or unsubstituted phenylene group, a substituted or unsubstituted biphenylene group, or a substituted or unsubstituted naphthylene group.

The phenylene group is an o-phenylene group, a m-phenylene group, or a p-phenylene group, and particularly preferably a p-phenylene group.

The biphenylene group is preferably a 4,2′-biphenylene group, a 4,3′-biphenylene group, a 4,4′-biphenylene group, or a 3,3′-biphenylene group, more preferably a 4,3′-biphenylene group, a 4,4′-biphenylene group, or a 3,3′-biphenylene group, and still more preferably a 4,4′-biphenylene group.

The naphthylene group is preferably a 1,4-naphthylene group, a 2,6-naphthylene group, a 1,5-naphthylene group, or a 1,8-naphthylene group.

The details of the substituted or unsubstituted divalent heterocyclic group having 5 to 30 ring atoms represented by L¹ to L⁴ are as described above in the section of “Substituents in Description”.

In the formula (1),
in the case where Ar² is represented by the formula (2A), it is preferable that any one of R³¹ to R³⁸, R^{C}, and R^{D} is a single bond bonded to **, a monocyclic ring or a condensed ring formed of a pair of groups adjacent to each other among R³¹ to R³⁸ is bonded to **, or a monocyclic ring or a condensed ring formed of R^{C} and R^{D} is bonded to **;
in the case where Ar² is represented by the formula (2B), it is preferable that one selected from R⁴¹ to R⁴⁴ which are not bonded to *c and *d, R⁴⁵ to R⁴⁸ which are not bonded to *a and *b, R⁵¹ to R⁵⁴, R⁵⁵ to R⁵⁸, and R^{X} is a single bond bonded to **, or a monocyclic ring or a condensed ring formed of a pair of groups adjacent to each other among R⁴¹ to R⁴⁴ which are not bonded to *c and *d, R⁴⁵ to R⁴⁸ which are not bonded to *a and *b, R⁵¹ to R⁵⁴, and R⁵⁵ to R⁵⁸ is bonded to **;
in the case where Ar² is represented by the formula (2C), it is preferable that one selected from R⁶¹ to R⁶⁸, and R^{Y} is a single bond bonded to **;
in the case where Ar² is represented by the formula (2D), it is preferable that any one of R⁷¹ to R⁷⁸, R^{E}, and R^{F} which are not a hydrogen atom is a single bond bonded to **, a monocyclic ring or a condensed ring formed of a pair of groups adjacent to each other among R⁷¹ to R⁷⁸ is bonded to **, or a monocyclic ring or a condensed ring formed of R^{E} and R^{F} is bonded to **;
in the case where Ar² is represented by the formula (2E), it is preferable that any one of R⁸¹, R⁸², R⁸⁴, R⁸⁵, R⁸⁷, R⁸⁸, and R^{Z} is a single bond bonded to **; and
in the case where Ar² is represented by the formula (2F), it is preferable that any one of R⁹¹, R⁹³ to R⁹⁶, R⁹⁸, R^{G}, and R which are not a hydrogen atom is a single bond bonded to Ar¹, a monocyclic ring or a condensed ring formed of a pair of groups adjacent to each other among R⁹¹, R⁹³ to R⁹⁶, R⁹⁸, Y^{C}, and Y^{D} is bonded to Ar¹, or a monocyclic ring or a condensed ring formed of R^{G} and R^{J} is bonded to Ar¹.

In other words, the compound (1) is represented by any one of combinations of the following formulae [a] to [l] below.
[a]: (1Aa)-(2C)
[b]: (1Aa)-(2D)
[c]: (1Aa)-(2E)
[d]: (1Aa)-(2F)
[e]: (1Ab)-(2A)
[f]: (1Ab)-(2B)
[g]: (1B)-(2A)
[h]: (1B)-(2B)
[i]: (1B)-(2C)
[j]: (1B)-(2D)
[k]: (1B)-(2E)
[l]: (1B)-(2F)

Among these, [a] to [h] are preferable, and [e] to [h] are more preferable.

In one embodiment, the compound (1) is represented by any one of the following formulae (1-1) to (1-4).

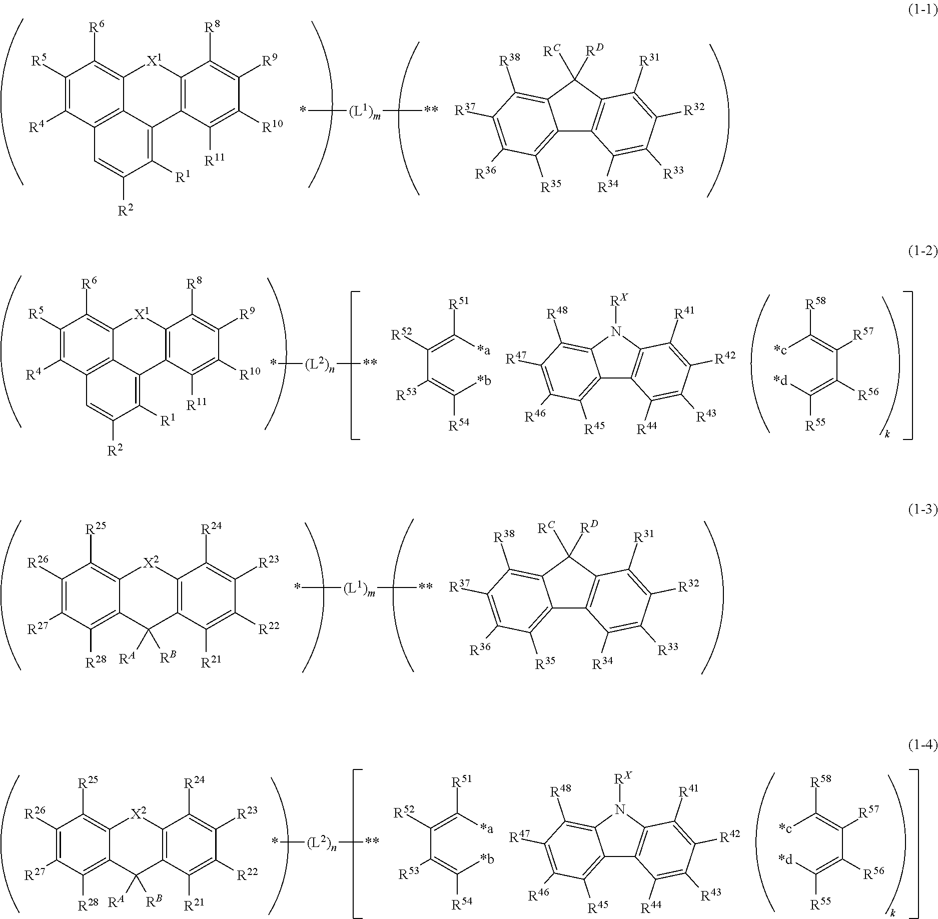

In the formulae (1-1) to (1-4), X¹, X², L¹, L², R^{A}, R^{B}, R^{C}, R^{D}, R^{X}, R¹ to R⁶, R⁸ to R¹¹, R²¹ to R²⁸, R³¹ to R³⁸, R⁴¹ to R⁴⁸, R⁵¹, R⁵⁴, R⁵⁵ to R⁵⁸, k, m, n, *, **, *a, *b, *c, and *d are as defined in the formula (1).

The compound (1) is preferably represented by the formula (1-1) or (1-2).

In addition, in one embodiment, the compound (1) is represented by any one of the following formulae (1-5) to (1-8).

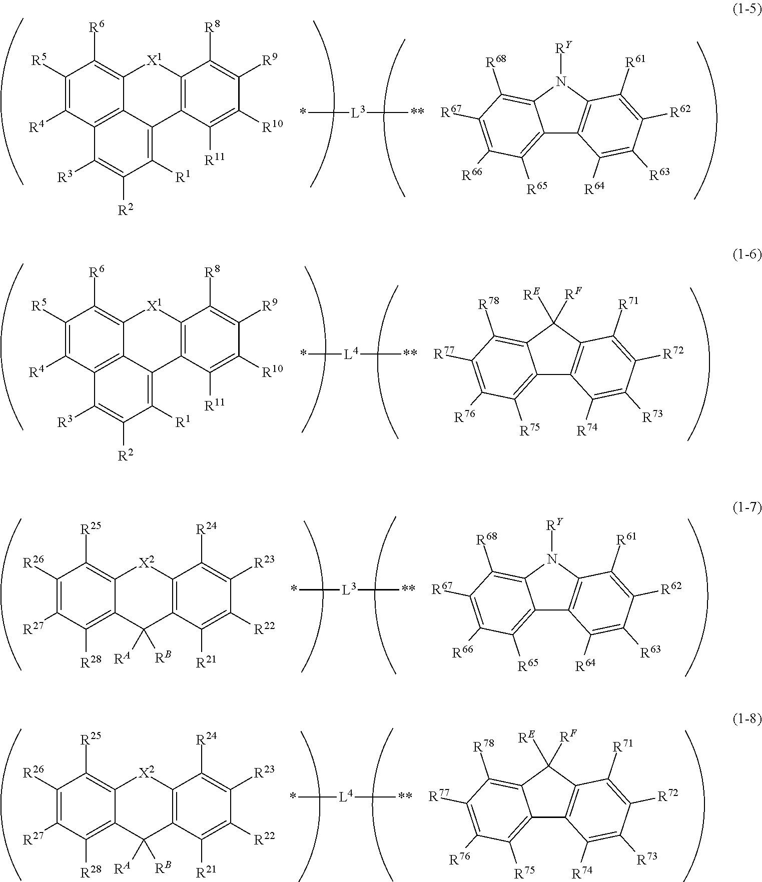

In the formulae (1-5) to (1-8), X¹, X², L³, L⁴, R^{A}, R^{B}, R^{E}, R^{F}, R^{Y}, R¹ to R⁶, R⁸ to R¹¹, R²¹ to R²⁸, R⁶¹ to R⁶⁸, R⁷¹ to R⁷⁸, *, and ** are as defined in the formula (1).

The compound (1) is preferably represented by the formula (1-5) or (1-6).

In one embodiment, in the above formulae (1-5) and (1-7), one selected from R⁶¹, R⁶², R⁶⁴, R⁶⁵, R⁶⁷, R⁶⁸, and R^{Y} is a single bond bonded to ** or a group bonded to **.

In one embodiment, the compound (1) is represented by any one of the following formulae (1-5a) to (1-5d) and (1-7a) to (1-7d).

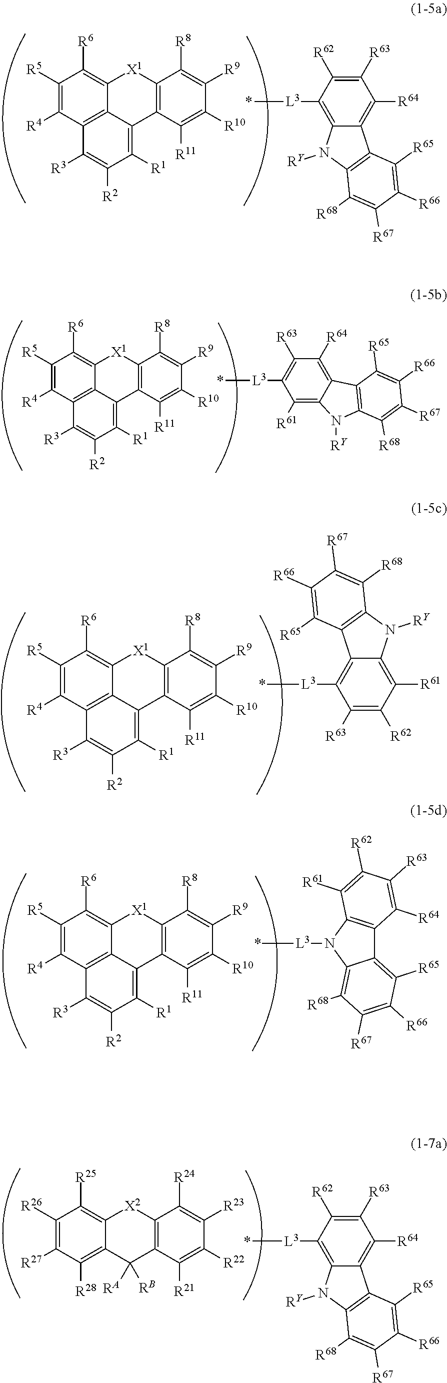

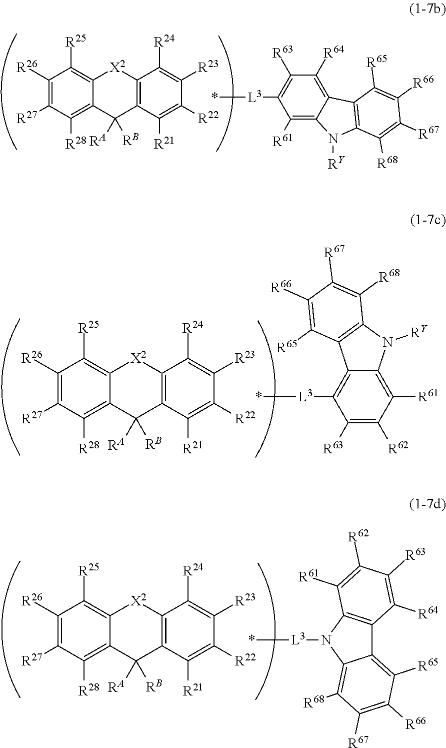

In the formulae (1-5a) to (1-5d) and (1-7a) to (1-7d), X¹, X², L³, R^{A}, R^{B}, R^{Y}, R¹ to, R⁶, R⁸ to R¹¹, R²¹ to R²⁸, R⁶¹ to R⁶⁸, and * are as defined in the formula (1).

Further, in one embodiment, in the above formulae (1-6) and (1-8), one selected from R⁷¹, R⁷³ to R⁷⁶, R⁷⁸, R^{E}, and R^{F} is a single bond bonded to ** or a group bonded to **.

In one embodiment, the compound (1) is represented by any one of the following formulae (1-6a) to (1-6c) and (1-8a) to (1-8c).

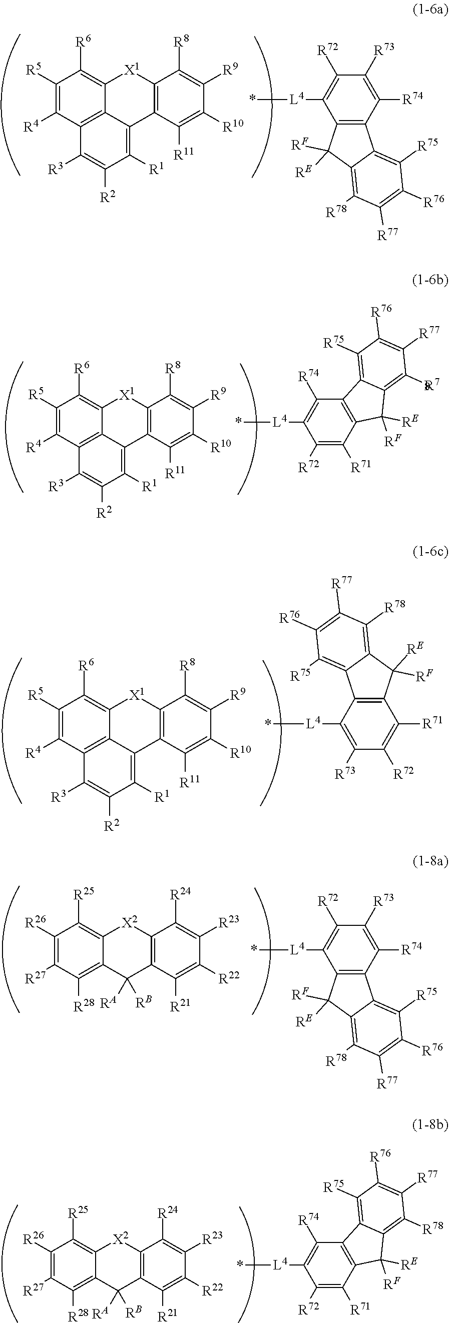

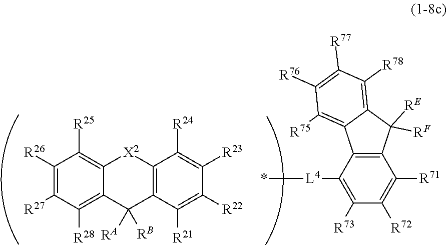

In the formulae (1-6a) to (1-6c) and (1-8a) to (1-8c), X¹, X², L⁴, R^{A}, R^{B}, R^{E}, R^{F}, R¹ to R⁶, R⁸ to R¹¹, R²¹ to R²⁸, R⁶¹ to R⁶⁸, and * are as defined in the formula (1).

In addition, in one embodiment, the compound (1) is represented by any one of the following formulae (1-9) to (1-12).

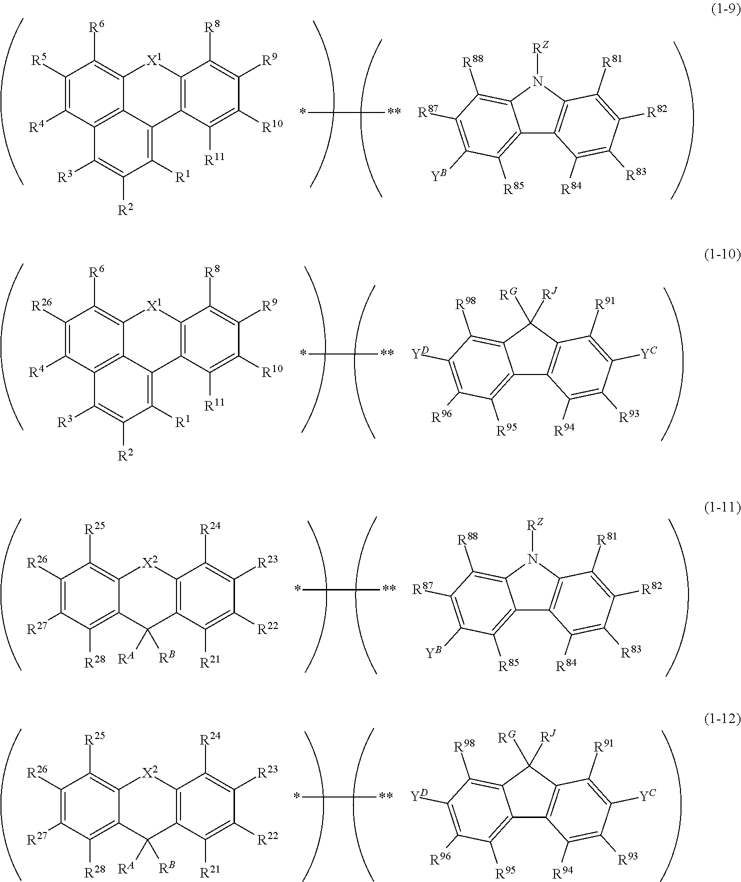

In the formulae (1-9) to (1-12), X¹, X², R^{A}, R^{B}, R^{G}, R^{Y}, R^{Z}, R¹ to R⁶, R⁸ to R¹¹, R²¹ to R²⁸, R⁸¹, R⁸², R⁸⁴, R⁸⁵, R⁸⁷, R⁸⁸, R⁹¹, R⁹³ to R⁹⁶, R⁹⁸, Y^{A}, Y^{B}, Y^{C}, Y^{D}, *, and ** are as defined in the formula (1).

The compound (1) is preferably represented by the formula (1-9) or (1-10).

In one embodiment, the compound (1) is represented by any one of the following formulae (1-9a) to (1-9d) and (1-11a) to (1-11d).

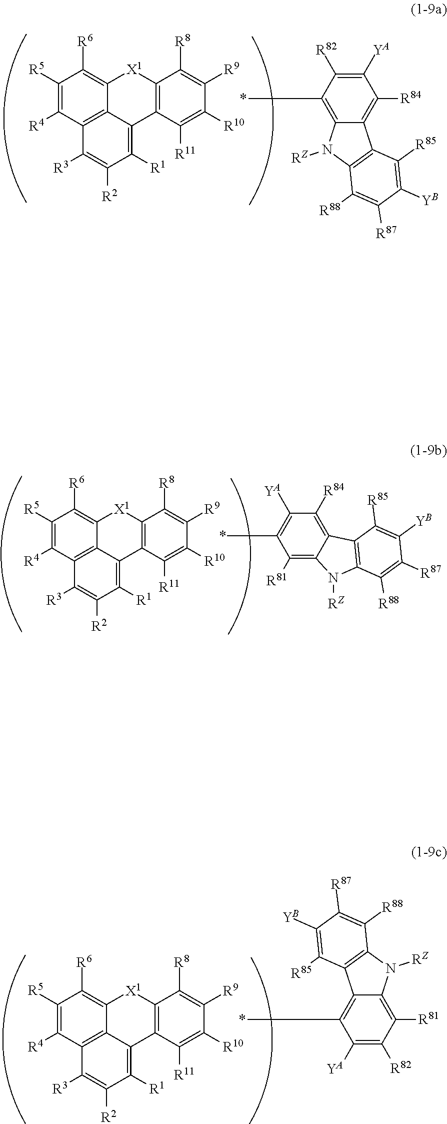

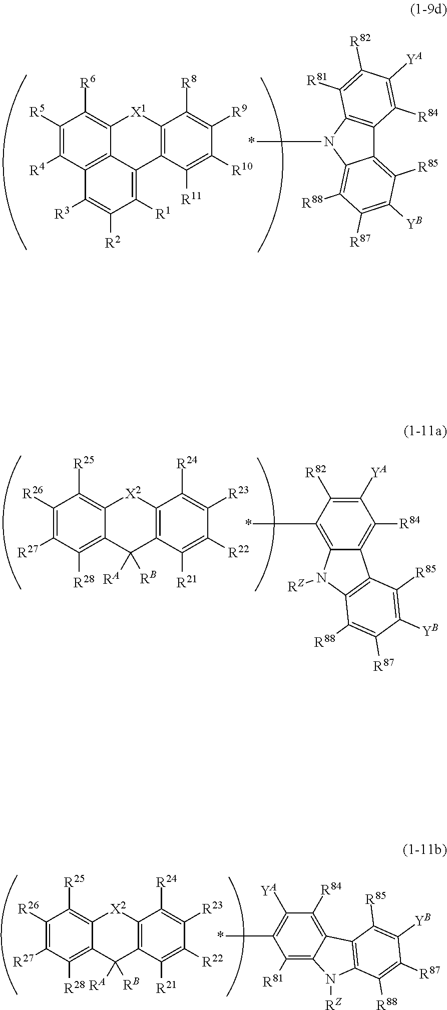

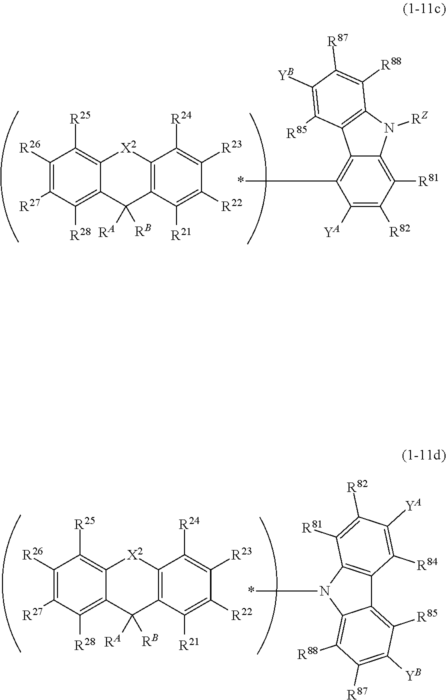

In the formulae (1-9a) to (1-9d) and (1-11a) to (1-11d), X¹, X², R^{A}, R^{B}, R^{Z}, R¹ to R⁶, R⁸ to R¹¹, R²¹ to R²⁸, R⁸¹, R⁸², R⁸⁴, R⁸⁵, R⁸⁷, R⁸⁸, Y^{A}, Y^{B}, and * are as defined in the formula (1).

In one embodiment, the compound (1) is represented by any one of the following formulae (1-10a) to (1-10c) and (1-12a) to (1-12c).

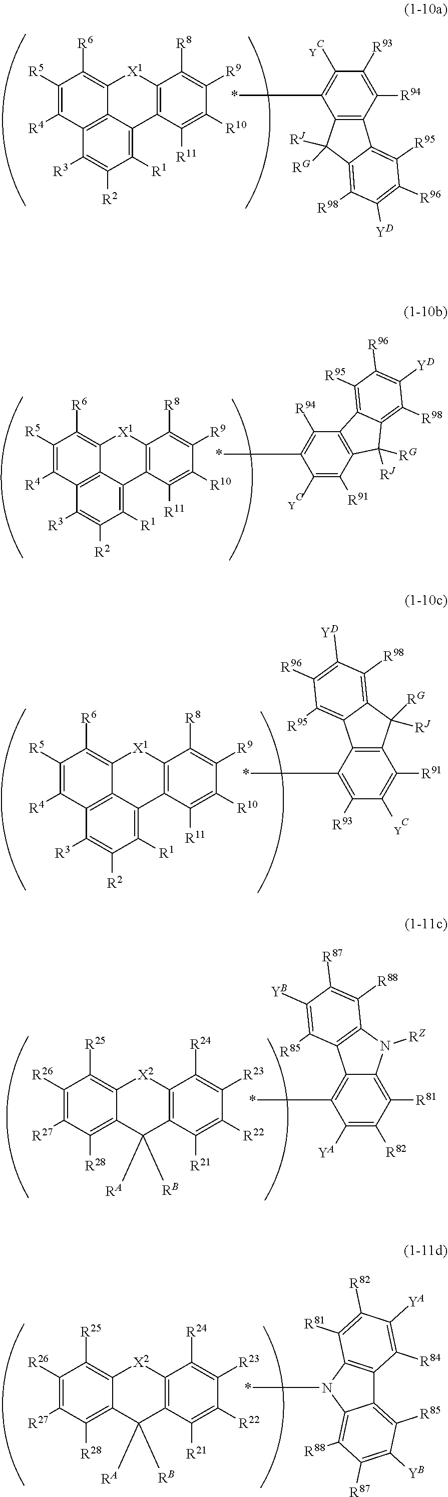

In the formulae (1-10a) to (1-10c) and (1-12a) to (1-12c), X¹, X², R^{G}, R^{J}, R¹ to R⁶, R⁸ to R¹¹, R²¹ to R²⁸, R⁹¹, R⁹³ to R⁹⁶, R⁹⁸, Y^{C}, Y^{D}, and * are as defined in the formula (1).

In one embodiment, the compound (1) is represented by any one of the following formulae (1-1-1), (1-2-1), (1-5-1), (1-6-1), (1-9-1), and (1-10-1).

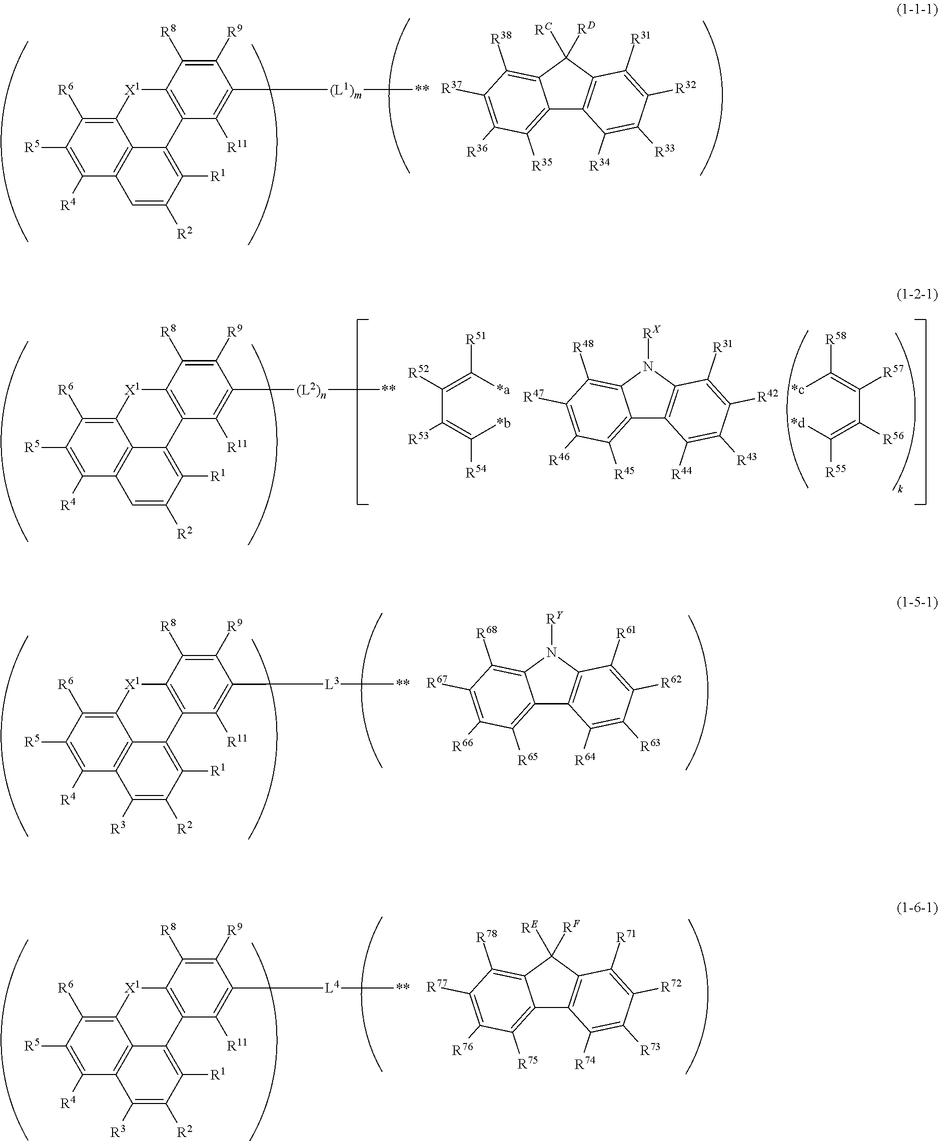

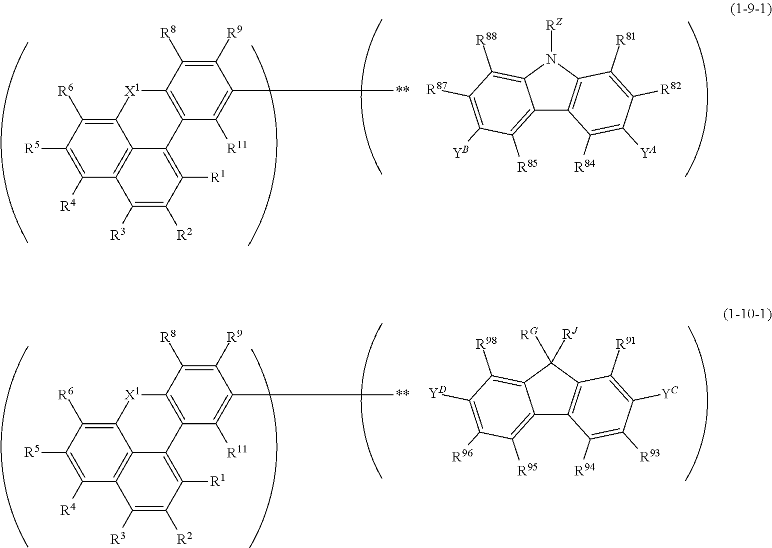

In the formulae (1-1-1), (1-2-1), (1-5-1), (1-6-1), (1-9-1), and (1-10-1), X¹, L¹, L², L³, L⁴, R^{C}, R^{D}, R^{E}, R^{F}, R^{G}, R^{J}, R^{X}, R^{Y}, R^{Z}, R¹ to R⁶, R⁸ to R¹¹, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹³ to R⁹⁶, R⁹⁸, Y^{A}, Y^{B}, Y^{C}, Y^{D}, k, m, n, **, *a, *b, *c, and *d are as defined in the formula (1).

In one embodiment, in the above formulae (2A), (1-1), (1-3), and (1-1-1), m is 0, or m is 1 and L¹ is a phenylene group or a biphenylene group.

In one embodiment, in the above formulae (2B), (1-2), (1-4), and (1-2-1), n is 0, or n is 1 and L² is a phenylene group or a biphenylene group.

In one embodiment, in the above formulae (1Aa), (1Ab), (1-1), (1-2), (1-5), (1-6), (1-9), (1-10), (1-9a) to (1-9d), (1-10a) to (1-10c), (1-1-1), (1-2-1), (1-5-1), (1-6-1), (1-9-1), and (1-10-1), X¹ is an oxygen atom.

In one embodiment, in the above formulae (1), (1-3), (1-4), (1-4), (1-7), (1-8), (1-11), (1-12), (1-11a) to (1-11d), and (1-12a) to (1-12c), X² is an oxygen atom.

In one embodiment, in the formula (2A), one selected from R³², R³⁴, R³⁵, and R³⁷ is a single bond bonded to ** or a group bonded to **; in the formula (2D), one selected from R⁷², R⁷⁴, R⁷⁵, and R⁷⁷ is a single bond bonded to ** or a group bonded to **; and in the formula (2F), one selected from R⁹⁴ and R⁹⁵ is a single bond bonded to ** or a group bonded to **.

In other words, in one embodiment, the 2-position or the 4-position on the fluorene skeleton in the formulae (2A) and (2D) is bonded to **, and the 4-position on the fluorene skeleton in the formula (2F) is bonded to **.

In one embodiment, the formula (1Aa) is represented by the following formulae (1Aa-1) to (1Aa-10).

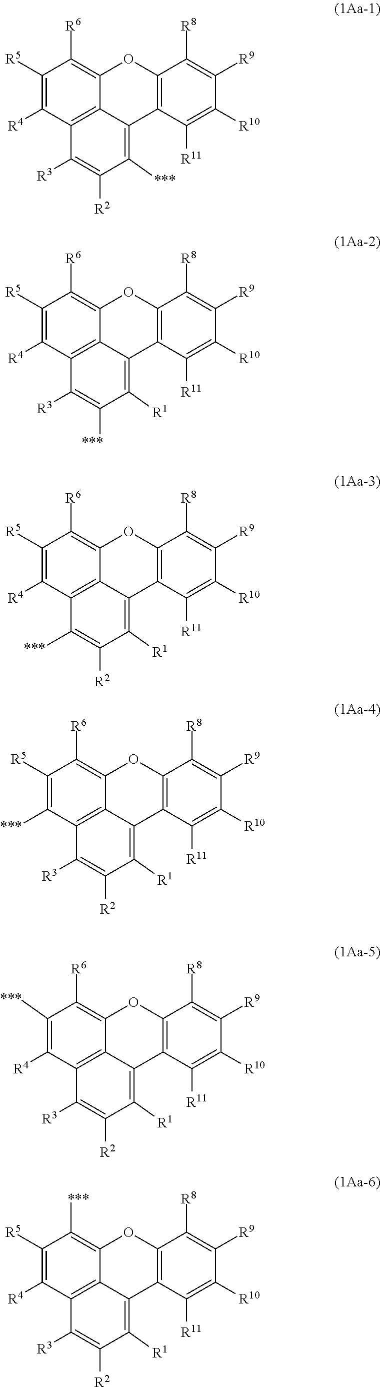

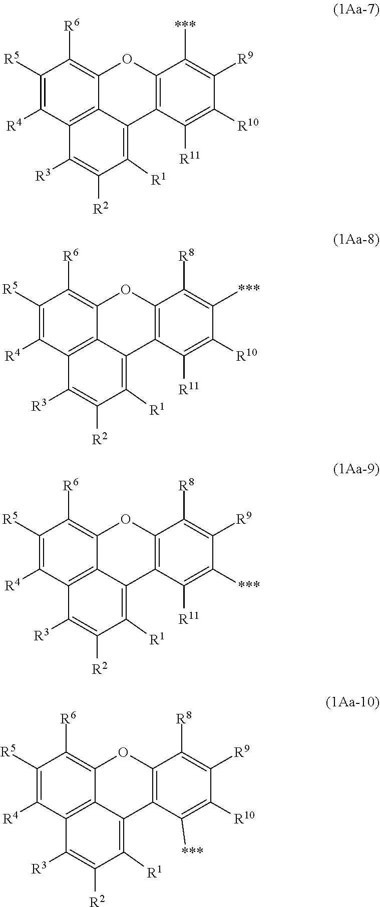

In the formulae (1Aa-1) to (1Aa-10), *** represents a bonding site to Ar², R¹ to R⁶ and R⁸ to R¹¹ are as defined in the formula (1).

In one embodiment, the formula (1Aa) is represented by the following formulae (1Aa-11) to (1Aa-20).

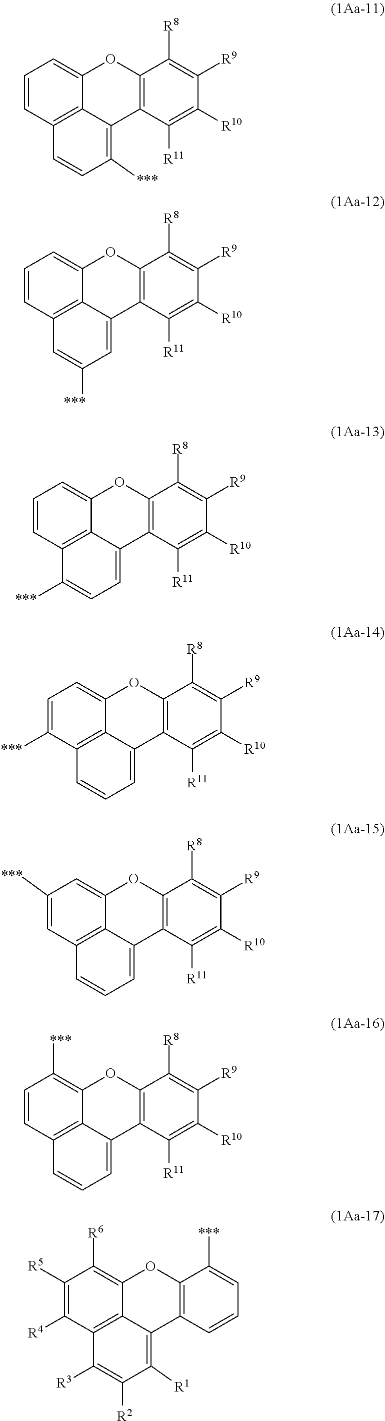

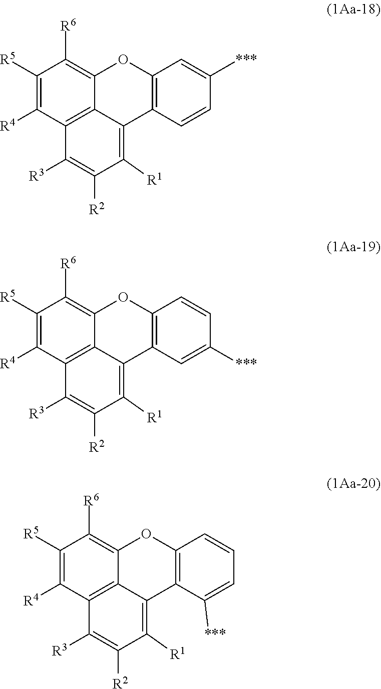

In the formulae (1Aa-11) to (1Aa-20), *** represents a bonding site to Ar², R¹ to R⁶ and R⁸ to R¹¹ are as defined in the formula (1).

In one embodiment, the formula (1Ab) is represented by the following formulae (1Ab-1) to (1Ab-9).

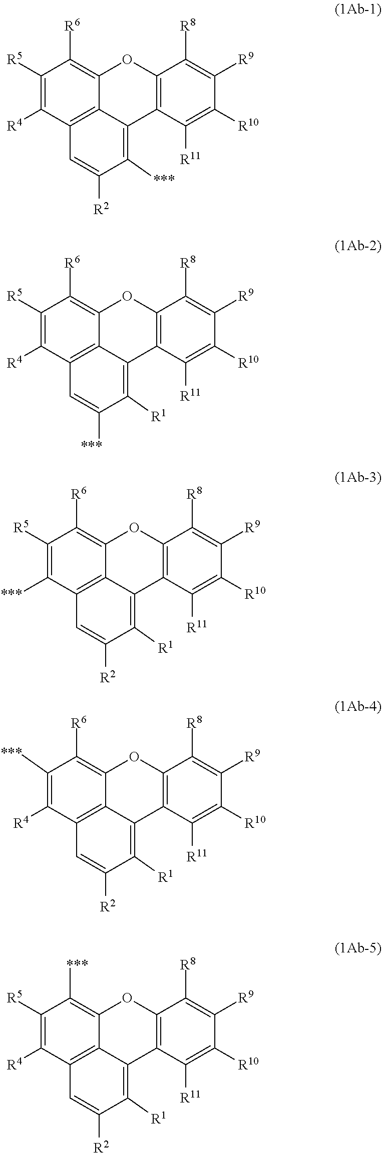

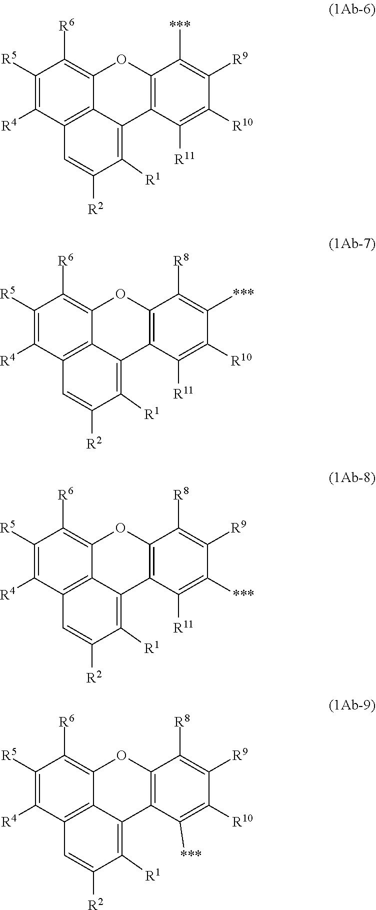

In the formulae (1Ab-1) to (1Ab-9), *** represents a bonding site to Ar², R¹, R², R⁴ to R⁶, and R⁸ to R¹¹ are as defined in the formula (1).

In one embodiment, the formula (1Ab) is represented by the following formulae (1Ab-11) to (1Ab-19).

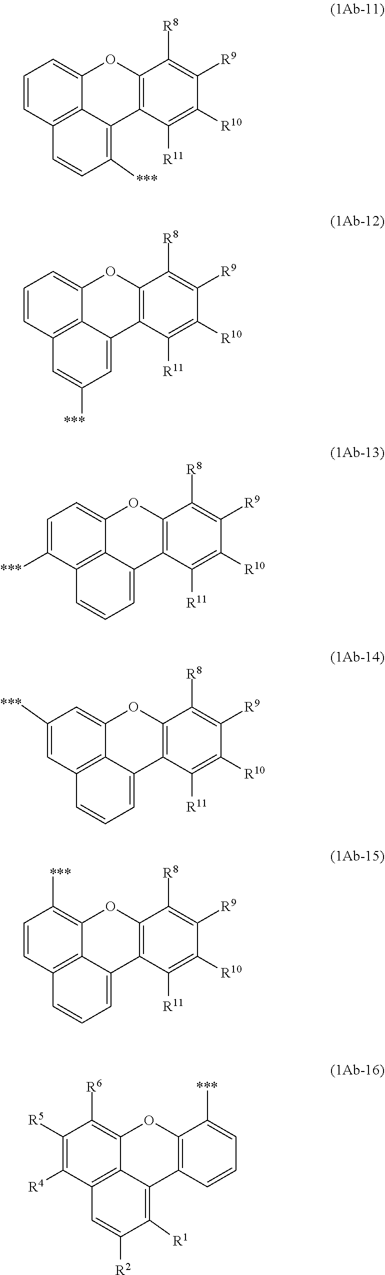

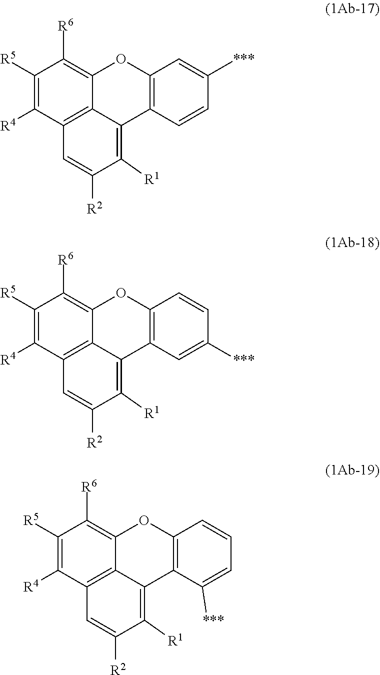

In the formulae (1Ab-1) to (1Ab-9), *** represents a bonding site to Ar², R¹, R², R⁴ to R⁶, and R⁸ to R¹¹ are as defined in the formula (1).

In one embodiment, in the formulae (1), (1-3), (1-4), (1-7), (1-8), (1-11), (1-12), and (1-11a) to (1-11d), R^{A} and R^{B} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group.

In one embodiment, in the formulae (1B), (1-3), (1-4), (1-7), (1-8), (1-11), (1-12), and (1-11a) to (1-11d), R^{A} and R^{B} are bonded to each other to form a substituted or unsubstituted monocyclic ring, or are bonded to each other to form a substituted or unsubstituted condensed ring.

The unsubstituted monocyclic ring formed by R^{A} and R^{B} is, for example, a benzene ring, a cyclopentane ring, or a cyclohexane ring.

The unsubstituted condensed ring formed by R^{A} and R^{B} is, for example, a naphthalene ring or an anthracene ring.

In addition, in the case where R^{A} and R^{B} are bonded to each other to form an unsubstituted monocyclic ring or an unsubstituted condensed ring, R^{A} and R^{B} may form a ring together with a xanthene skeleton to which these are bonded, for example, a spirobixanthene skeleton or a spiro[fluorene-9,9′-xanthene]skeleton.

In one embodiment, the formula (1B) is represented by the following formulae (1B-1) to (1B-5).

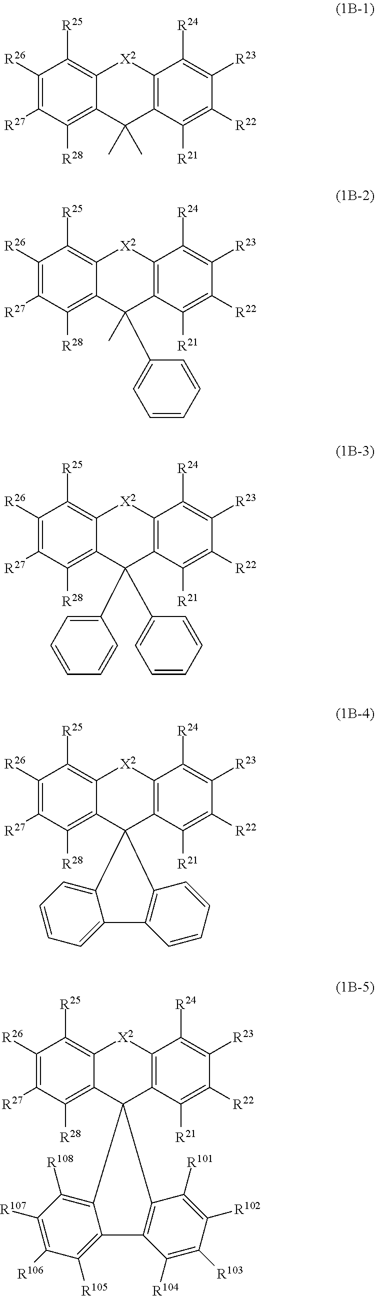

In the formulae (1B-1) to (1B-5), X² and R²¹ to R²⁸ are as defined in the formula (1).

In the formula (1B-5), R¹⁰¹ to R¹⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (1B-5), one selected from R²¹ to R²⁸ and R¹⁰¹ to R¹⁰⁸ is a single bond bonded to Ar² or a group bonded to Ar².

In one embodiment, in the above formulae (2A), (1-1), (1-3) and (1-1-1), R^{C} and R^{D} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group. Preferably, R^{C} and R^{D} are each a phenyl group, one of R^{C} and R^{D} is a phenyl group, and the other is a naphthyl group.

In one embodiment, in the above formulae (2D), (1-6), (1-8) and (1-6-1), R^{E} and R^{F} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group. Preferably, R^{E} and R^{F} are each a phenyl group, one of R^{E} and R^{F} is a phenyl group, and the other is a naphthyl group.

In one embodiment, in the above formulae (2F), (1-10), (1-12), (1-10a) to (1-10c), (1-12a) to (1-12c), and (1-10-1), R^{G} and R^{J} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group. Preferably, R^{G} and R^{J} are each a phenyl group, one of R^{G} and R^{J} is a phenyl group, and the other is a naphthyl group.

In one embodiment, in the formulae (2A), (1-1), (1-3), and (1-1-1), R^{C} and R^{D} are bonded to each other to form a substituted or unsubstituted monocyclic ring, or are bonded to each other to form a substituted or unsubstituted condensed ring.

In one embodiment, in the formulae (2D), (1-6), (1-8), and (1-6-1), R^{E} and R^{F} are bonded to each other to form a substituted or unsubstituted monocyclic ring, or are bonded to each other to form a substituted or unsubstituted condensed ring.

In one embodiment, in the formulae (2F), (1-10), (1-12), (1-10a) to (1-10c), (1-12a) to (1-12c), and (1-10-1), R^{G} and R^{J} are bonded to each other to form a substituted or unsubstituted monocyclic ring, or are bonded to each other to form a substituted or unsubstituted condensed ring.

The unsubstituted monocyclic ring formed by R^{C} and R^{D}, the unsubstituted monocyclic ring formed by R^{E} and R^{F}, and the unsubstituted monocyclic ring formed by R^{G} and R^{J} are, for example, a benzene ring, a cyclopentane ring, or a cyclohexane ring.

The unsubstituted condensed ring formed by R^{C} and R^{D}, the unsubstituted condensed ring formed by R^{E} and R^{F}, and the unsubstituted condensed ring formed by R^{G} and R^{J} are, for example, a naphthalene ring or an anthracene ring.

In addition, in the case where R^{C} and R^{D} are bonded to each other to form an unsubstituted monocyclic ring or an unsubstituted condensed ring, in the case where R^{E} and R^{F} are bonded to each other to form an unsubstituted monocyclic ring or an unsubstituted condensed ring, and in the case where R^{G} and R^{J} are bonded to each other to form an unsubstituted monocyclic ring or an unsubstituted condensed ring, R^{C} and R^{D}, R^{E} and R^{F}, and R^{G} and R^{J} form a ring together with a fluorene skeleton to which these are bonded, and for example, a spirobifluorene skeleton, a spiro[9H-fluorene-9,1′-cyclopentane] skeleton, a spiro[cyclohexane-1,9′-[9H]fluorene] skeleton, and a spiro[9H-fluorene-9,2′-tricyclo[3.3.1.1^{3,7}]decane] skeleton may be formed.

In one embodiment, Ar² represented by the formula (2A) is represented by any of the following formulae (2Aa) to (2Ag).

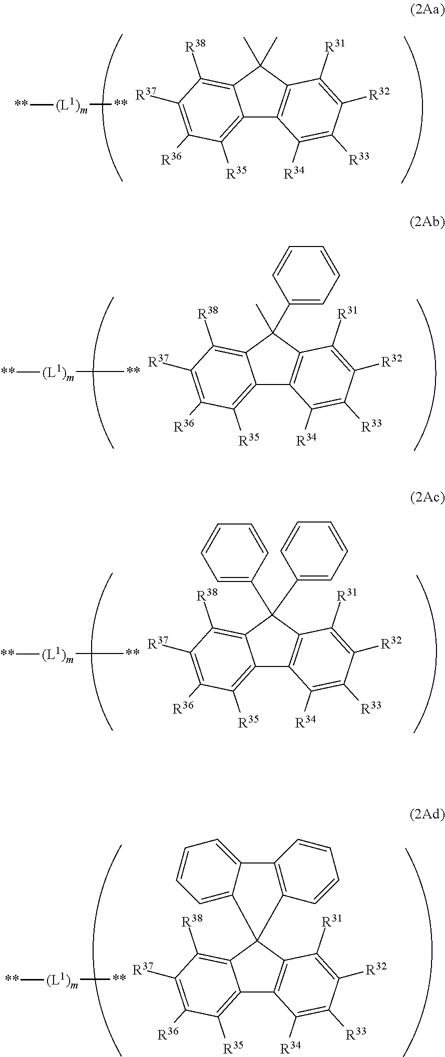

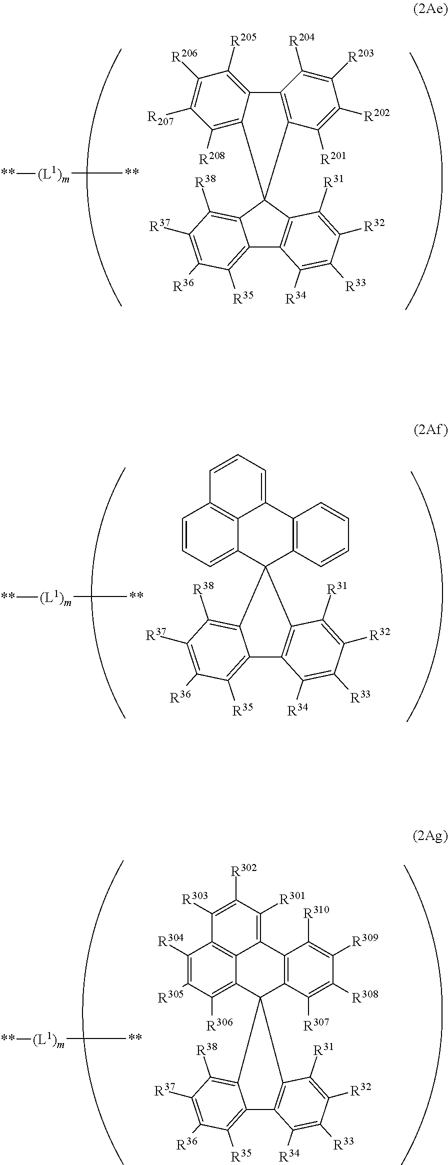

In the formulae (2Aa) to (2Ag), L¹, R³¹ to R³⁸, m, *, and ** are as defined in the formula (1).

In the formula (2Ae), R²⁰¹ to R²⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2Ae), one selected from R³¹ to R³⁸ and R²⁰¹ to R²¹⁰ is a single bond bonded to ** or a group bonded to Ar².

In the formula (2Ag), R³⁰¹ to R³¹⁰ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2Ag), one selected from R³¹ to R³⁸ and R³⁰¹ to R³¹⁰ is a single bond bonded to ** or a group bonded to Ar².

Among the alkyl group, the aryl group, and the heteroaryl group represented by R²⁰¹ to R²⁰⁸ and R³⁰¹ to R³¹⁰, preferred groups are the same as those described for R¹.

In one embodiment, Ar² represented by the formula (2B) is represented by any of the following formulae (2Ba) to (2Bi).

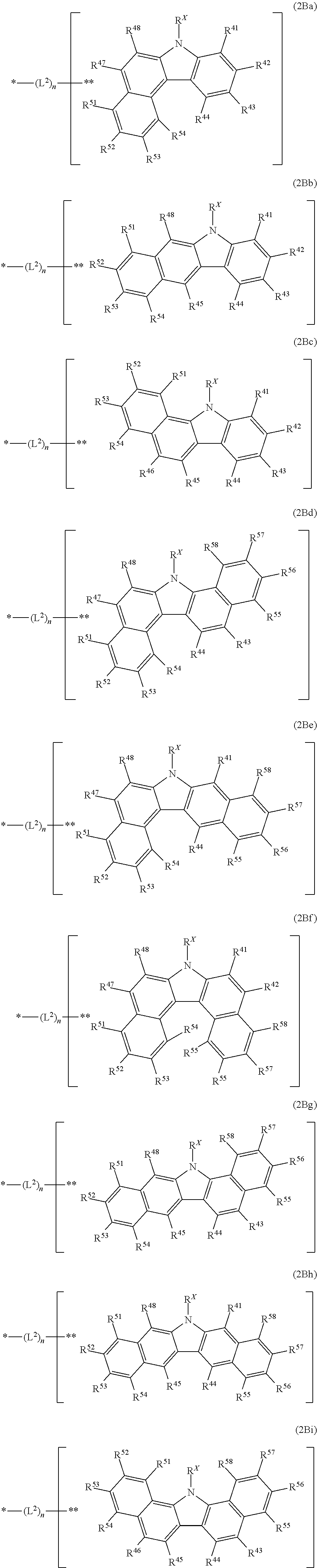

In the formulae (2Ba) to (2Bi), L², R^{X}, R⁴¹ to R⁴⁸, R⁵¹ to R⁵⁴, R⁵⁵ to R⁵⁸, n, *, and ** are as defined in the formula (1).

In one embodiment, Ar² represented by the formula (2D) is represented by any of the following formulae (2 Da) to (2Dj).

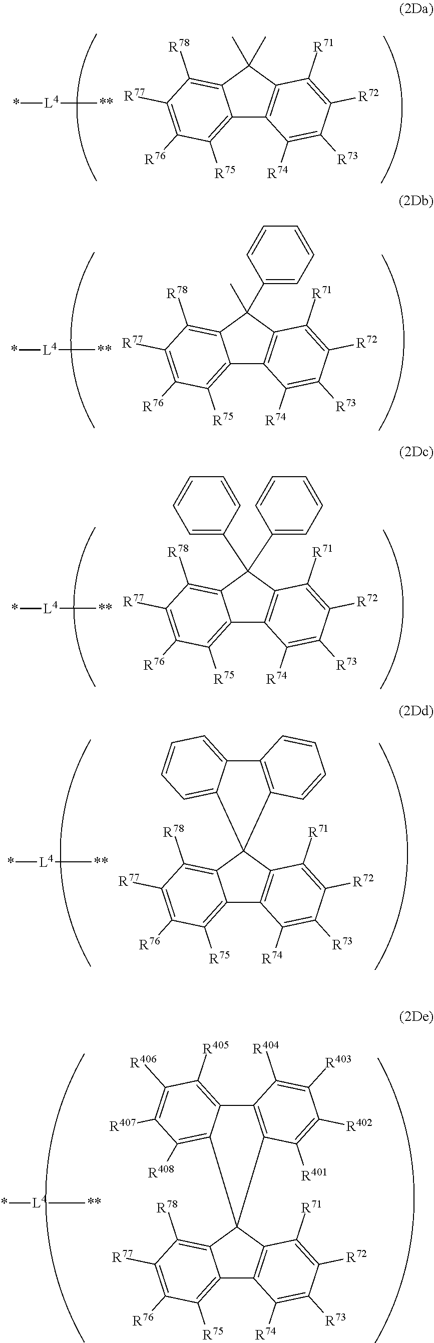

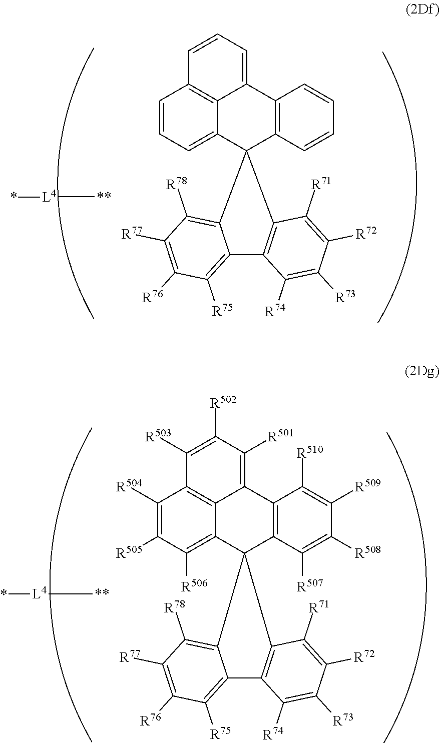

In the formulae (2 Da) to (2Dj), L⁴, R⁷¹ to R⁷⁸, *, and ** are as defined in the formula (1).

In the formula (2De), R⁴⁰¹ to R⁴⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2De), one selected from R⁷¹ to R⁷⁸ and R⁴⁰¹ to R⁴¹⁰ is a single bond bonded to ** or a group bonded to Ar².

In the formula (2Dg), R⁵⁰¹ to R⁵¹⁰ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2Dg), one selected from R⁷¹ to R⁷⁸ and R⁵⁰¹ to R⁵¹⁰ is a single bond bonded to ** or a group bonded to Ar².

Among the alkyl group, the aryl group, and the heteroaryl group represented by R⁴⁰¹ to R⁴⁰⁸ and R⁵⁰¹ to R⁵¹⁰, preferred groups are the same as those described for R¹.

In one embodiment, Ar² represented by the formula (2E) is represented by any of the following formulae (2Ea) to (2Ej).

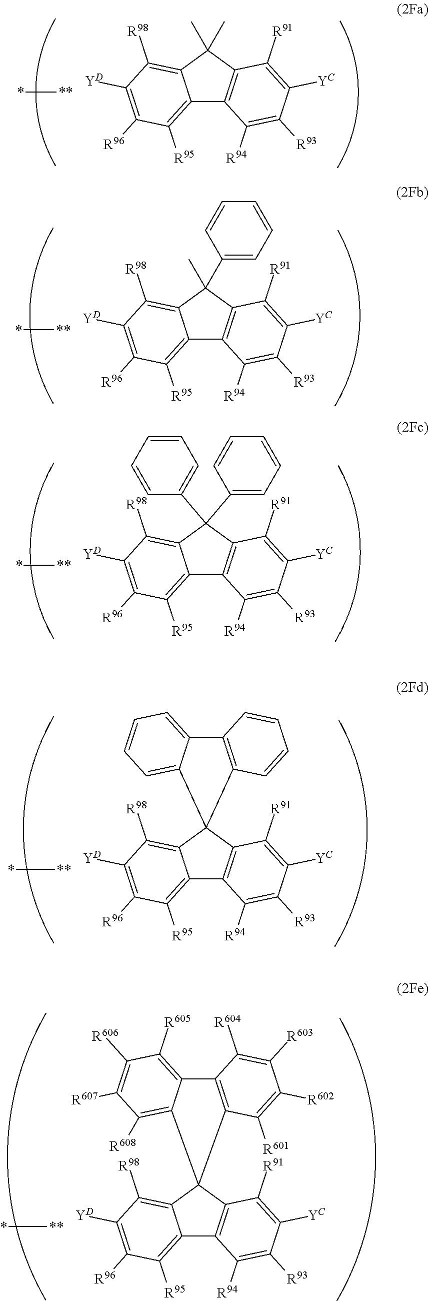

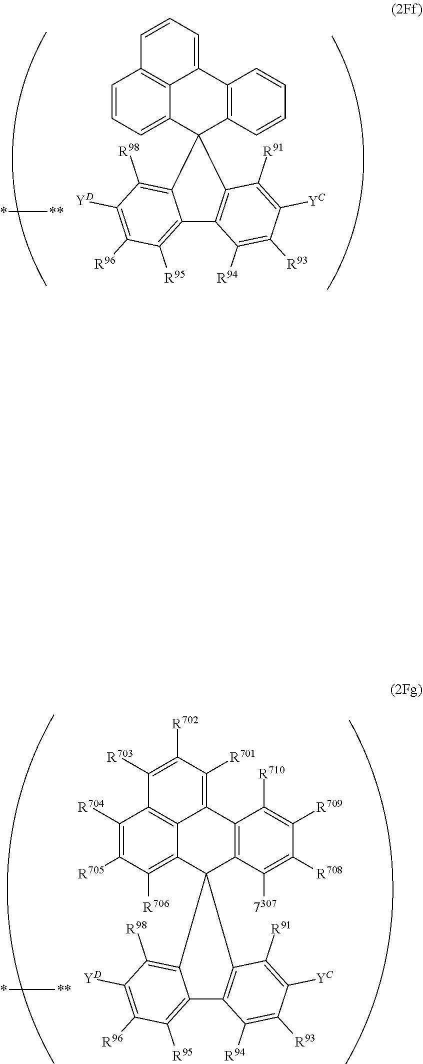

In the formulae (2Fa) to (2Fj), R⁹¹, R⁹², R⁹⁴, R⁹⁵, R⁹⁷, R⁹⁸, Y^{C}, Y^{D}, *, and ** are as defined in the formula (1).

In the formula (2Fe), R⁶⁰¹ to R⁶⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2Fe), one selected from R⁹¹, R⁹², R⁹⁴, R⁹⁵, R⁹⁷, R⁹⁸, Y^{C}, Y^{D} and R⁶⁰¹ to R⁶¹⁰ is a single bond bonded to ** or a group bonded to Ar².

In the formula (2Fg), R⁷⁰¹ to R⁷¹⁰ are each independently a hydrogen atom, a substituted or unsubstituted alkyl group having 1 to 6 carbon atoms, a substituted or unsubstituted alkyl group having 6 to 12 ring carbon atoms, or a substituted or unsubstituted heteroaryl group having 5 to 13 ring atoms.

In the formula (2Fg), one selected from R⁹¹, R⁹², R⁹⁴, R⁹⁵, R⁹⁷, R⁹⁸, Y^{C}, Y^{D} and R⁷⁰¹ to R⁷¹⁰ is a single bond bonded to ** or a group bonded to Ar².

Among the alkyl group, the aryl group, and the heteroaryl group represented by R⁶⁰¹ to R⁶⁰⁸ and R⁷⁰¹ to R⁷¹⁰, preferred groups are the same as those described for R¹.

In one embodiment, the compound (1) contains at least one deuterium atom.

The deuterium atom contained in the inventive compound (1) will be described in detail later.

In one embodiment of the inventive compound (1), at least one of the following (1) to (19) is a deuterium atom:
(1) a hydrogen atom represented by each of R¹ to R⁶, R⁸ to R¹¹, R²¹ to R²⁸, R³¹ to R³⁸, R⁴¹ to R⁴⁸, R¹ to R⁵⁴, R⁵⁵ to R⁵⁸, R⁶¹ to R⁶⁸, R⁷¹ to R⁷⁸, R⁸¹, R⁸², R⁵⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹⁰, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C}, and Y^{D};
(2) a hydrogen atom at the 3-position of the benzoxanthene skeleton of the formula (1Ab);
(3) a hydrogen atom directly bonded to an alkyl group represented by each of R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C}, and Y^{D};
(4) a hydrogen atom directly bonded to an aryl group represented by each of R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C} and Y^{D};
(5) a hydrogen atom directly bonded to a heteroaryl group represented by each of R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C} and Y^{D}.
(6) a hydrogen atom directly bonded to a substituent of an alkyl group represented by each of R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹⁰, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C}, and Y^{D};
(7) a hydrogen atom directly bonded to a substituent of an aryl group represented by each of R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹⁰, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C}, and Y^{D};
(8) a hydrogen atom directly bonded to a substituent of a heteroaryl group represented by each of R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R¹⁴, R⁸⁵, R⁸⁷, R⁸⁸, R⁹¹, R⁹², R⁹⁴, R⁹⁵, R⁹⁷, R⁹⁸, R¹⁰¹ to R¹⁰⁸, R²⁰¹ to R²⁰⁸, R³⁰¹ to R³¹⁰, R⁷¹ to R⁷⁸, R⁴⁰¹ to R⁴⁰⁸, R⁵⁰¹ to R⁵¹⁰, R⁶⁰¹ to R⁶⁰⁸, R⁷⁰¹ to R⁷¹⁰, Y^{A}, Y^{B}, Y^{C}, and Y^{D};
(9) a hydrogen atom represented by each of R^{A}, R^{B}, R^{C}, R^{D}, R^{E}, R^{F}, R^{G}, R^{J}, R^{X}, R^{Y}, and R^{Z};
(10) a hydrogen atom directly bonded to an alkyl group represented by each of R^{A}, R^{B}, R^{C}, R^{D}, R^{E}, R^{F}, R^{G}, R^{J}, R^{X}, R^{Y}, and R^{Z};
(11) a hydrogen atom directly bonded to an aryl group represented by each of R^{A}, R^{B}, R^{C}, R^{D}, R^{E}, R^{F}, R^{G}, R^{J}, R^{X}, R^{Y}, and R^{Z};
(12) a hydrogen atom directly bonded to a heteroaryl group represented by each of R^{A}, R^{B}, R^{C}, R^{D}, R^{E}, R^{F}, R^{G}, R^{J}, R^{X}, R^{Y}, and R^{Z};
(13) a hydrogen atom directly bonded to a substituent of an alkyl group represented by each of R^{A}, R^{B}, R^{C}, R^{D}, R^{E}, R^{F}, R^{G}, R^{J}, R^{X}, R^{Y}, and R^{Z};
(14) a hydrogen atom directly bonded to a substituent of an aryl group represented by each of R^{A}, R^{B}, R^{C}, R^{D}, R^{E}, R^{F}, R^{G}, R^{J}, R^{X}, R^{Y}, and R^{Z};
(15) a hydrogen atom directly bonded to a substituent of a heteroaryl group represented by each of R^{A}, R^{B}, R^{C}, R^{D}, R^{E}, R^{F}, R^{G}, R^{J}, R^{X}, R^{Y}, and R^{Z};
(16) a hydrogen atom directly bonded to an arylene group represented by each of L¹, L², L³, and L⁴;
(17) a hydrogen atom directly bonded to a divalent heterocyclic group represented by each of L¹, L², L³, and L⁴;
(18) a hydrogen atom directly bonded to a substituent of an arylene group represented by each of L¹, L², L³, and L⁴; and
(19) a hydrogen atom directly bonded to a substituent of a divalent heterocyclic group represented by each of L¹, L², L³, and L⁴.

As described above, the “hydrogen atom” used in the description herein includes a protium atom, a deuterium atom, and a tritium atom. The inventive compound may contain a naturally-derived deuterium atom.

A deuterium atom may be intentionally introduced into the inventive compound by using a deuterated compound as a part or the whole of the raw material compound.

The deuteration rate of the inventive compound depends on the deuteration rate of the raw material compound used. Even when a raw material having a predetermined deuteration rate is used, a naturally-derived protium isotope can be contained in a certain ratio. Accordingly, an embodiment of the deuteration rate of the inventive compound shown below includes the proportion for which a minor amount of a naturally-derived isotope is taken into consideration, relative to the proportion determined by counting the number of the deuterium atoms merely represented by a chemical formula.

The deuteration rate of the inventive compound is preferably 1% or more, more preferably 3% or more, still more preferably 5% or more, even more preferably 10% or more, and even more preferably 50% or more.

The inventive compound may be a deuterium compound in which all hydrogen atoms are deuterium atoms (that is, the deuteration rate of the inventive compound is 100%).

The inventive compound may be a mixture of a deuterated compound and a non-deuterated compound, or a mixture of two or more compounds having different deuteration rates from each other. The deuteration rate of the mixture is preferably 1% or more, more preferably 3% or more, still more preferably 5% or more, even more preferably 10% or more, and even more preferably 50% or more, and is less than 100%.

The proportion of the number of the deuterium atoms to the number of all the hydrogen atoms in the inventive compound is preferably 1% or more, more preferably 3% or more, still more preferably 5% or more, even more preferably 10% or more, and is 100% or less.

The details of the substituent (optional substituent) in the case of “substituted or unsubstituted” included in the definition of each of the above formulae are as described in the “Substituent for “Substituted or Unsubstituted”. The same applies to each formula relating to “compound A” described later.

The inventive compound and the “compound A” described later can be easily produced by a person skilled in the art with reference to Synthesis Examples described later and known synthesis methods.

Specific examples of the inventive compound will be described below, but the inventive compound is not limited to the following example compounds.

In the following specific examples, D represents a deuterium atom.

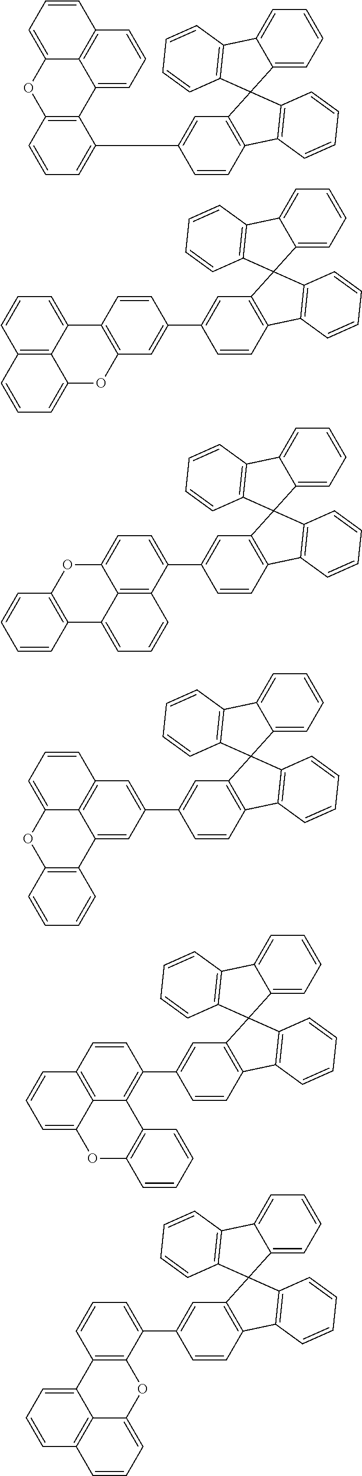

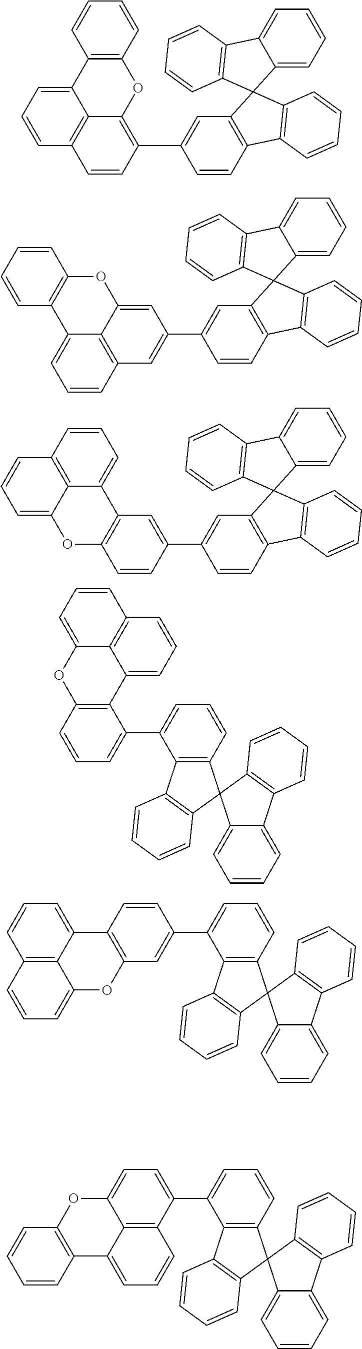

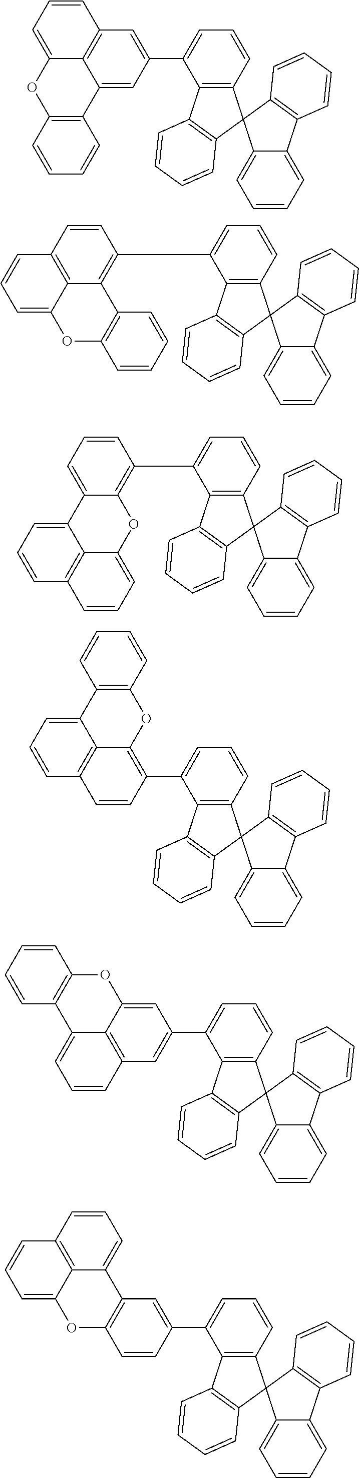

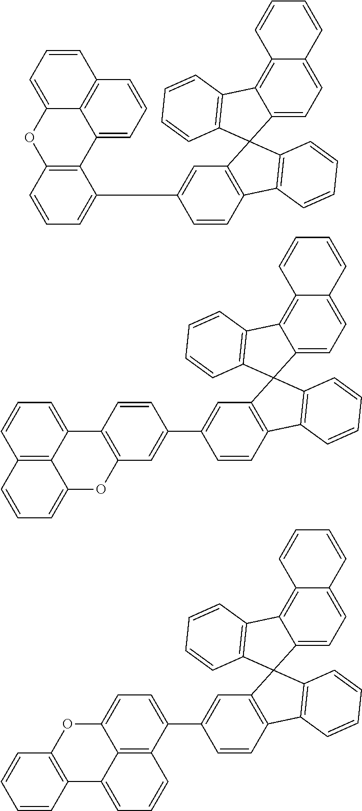

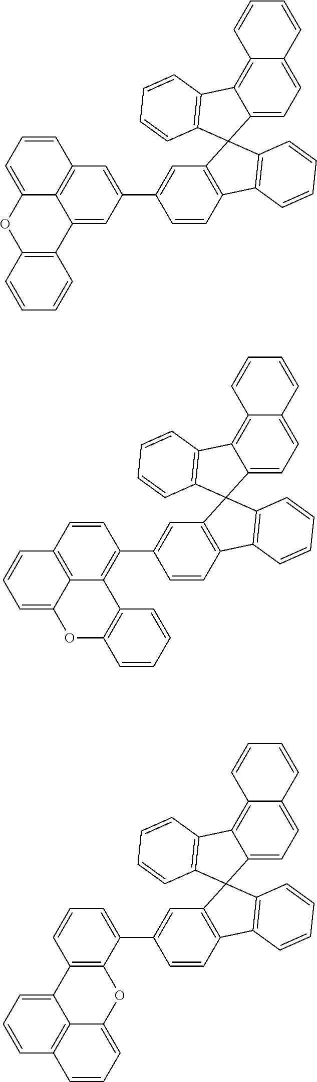 [SMILES: c1ccc2c(c1)Oc1cccc3cc(-c4ccc5c(c4)C4(c6ccccc6-5)c5ccccc5-c5c4ccc4ccccc54)cc-2c13 ; c1ccc2c(c1)Oc1cccc3ccc(-c4ccc5c(c4)C4(c6ccccc6-5)c5ccccc5-c5c4ccc4ccccc54)c-2c13 ; c1ccc2c(c1)-c1ccc(-c3cccc4c3Oc3cccc5cccc-4c35)cc1C21c2ccccc2-c2c1ccc1ccccc21]

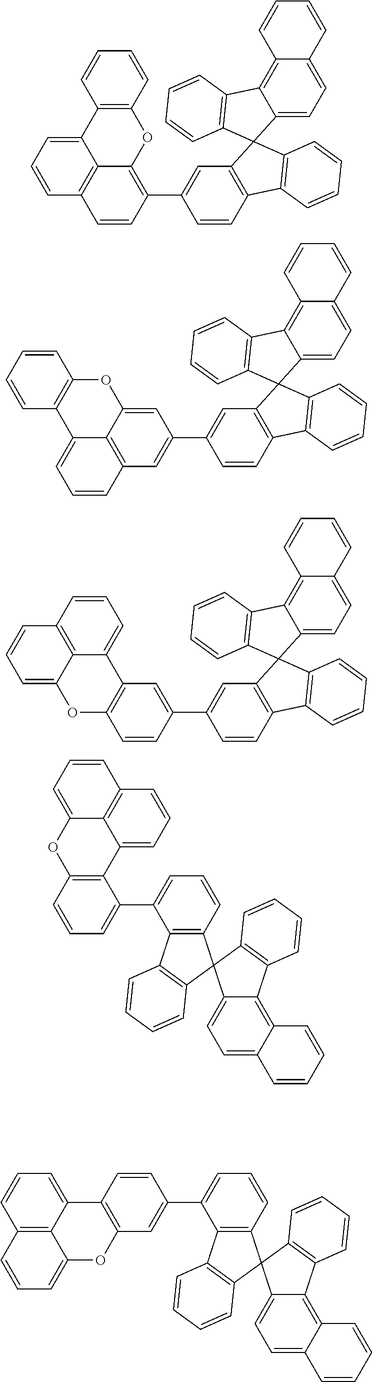

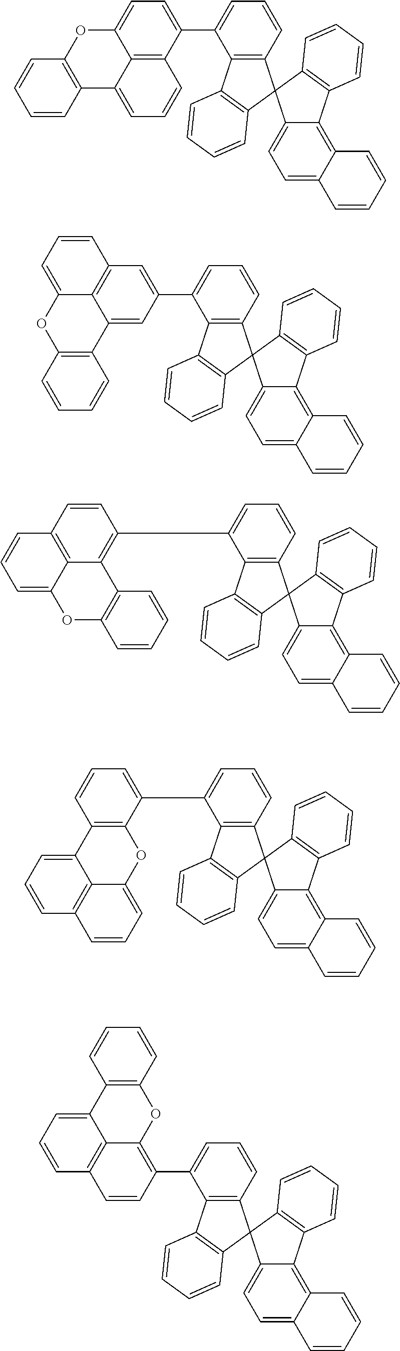

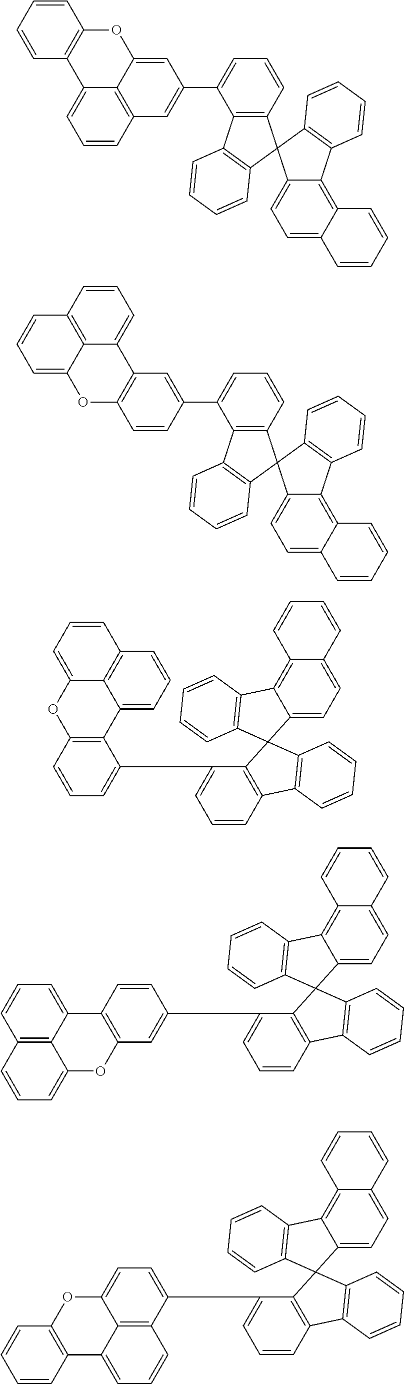

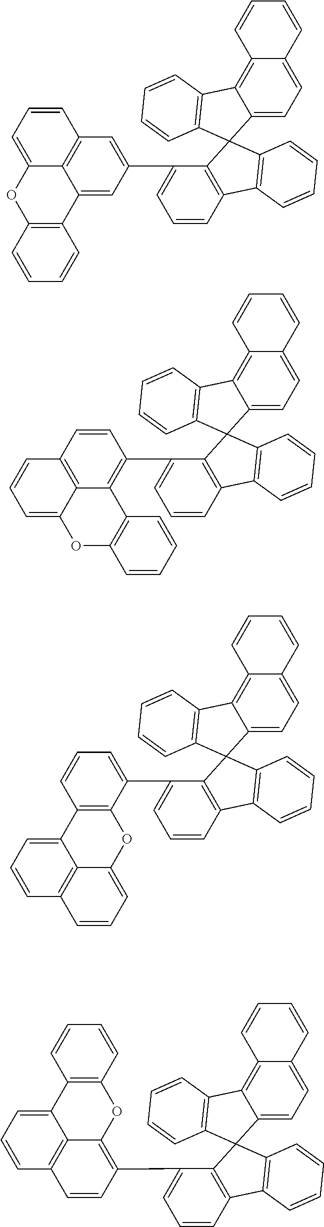 [SMILES: c1ccc2c(c1)Oc1cccc3cc(-c4cccc5c4C4(c6ccccc6-5)c5ccccc5-c5c4ccc4ccccc54)cc-2c13 ; c1ccc2c(c1)Oc1cccc3ccc(-c4cccc5c4C4(c6ccccc6-5)c5ccccc5-c5c4ccc4ccccc54)c-2c13 ; c1ccc2c(c1)-c1cccc(-c3cccc4c3Oc3cccc5cccc-4c35)c1C21c2ccccc2-c2c1ccc1ccccc21 ; c1ccc2c(c1)Oc1c(-c3cccc4c3C3(c5ccccc5-4)c4ccccc4-c4c3ccc3ccccc43)ccc3cccc-2c13]

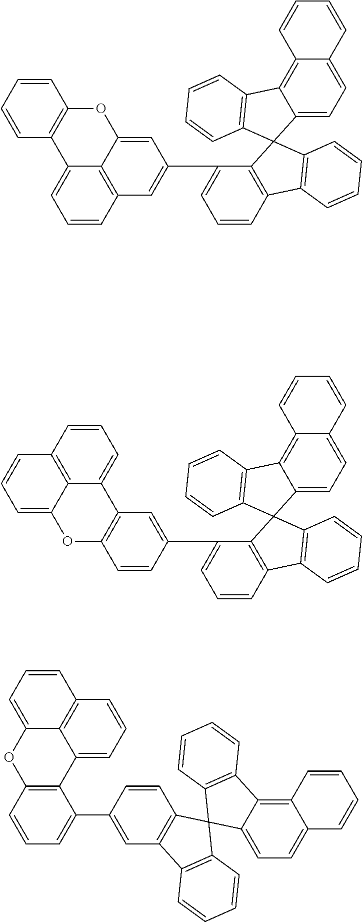 [SMILES: c1ccc2c(c1)Oc1cc(-c3cccc4c3C3(c5ccccc5-4)c4ccccc4-c4c3ccc3ccccc43)cc3cccc-2c13 ; c1ccc2c(c1)-c1cccc(-c3ccc4c(c3)-c3cccc5cccc(c35)O4)c1C21c2ccccc2-c2c1ccc1ccccc21 ; c1ccc2c(c1)-c1cc(-c3cccc4c3-c3cccc5cccc(c35)O4)ccc1C21c2ccccc2-c2c1ccc1ccccc21]

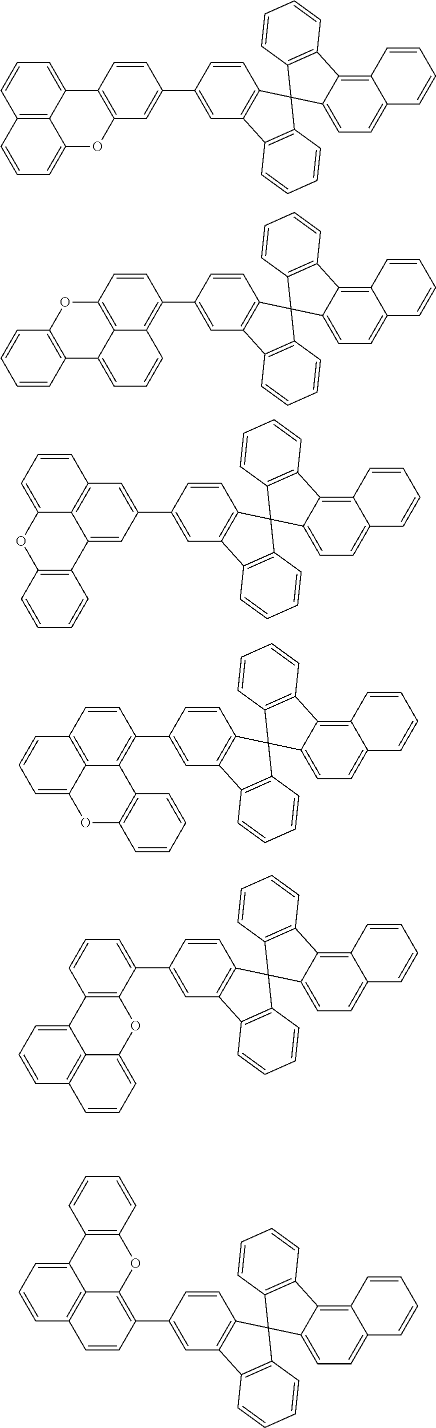

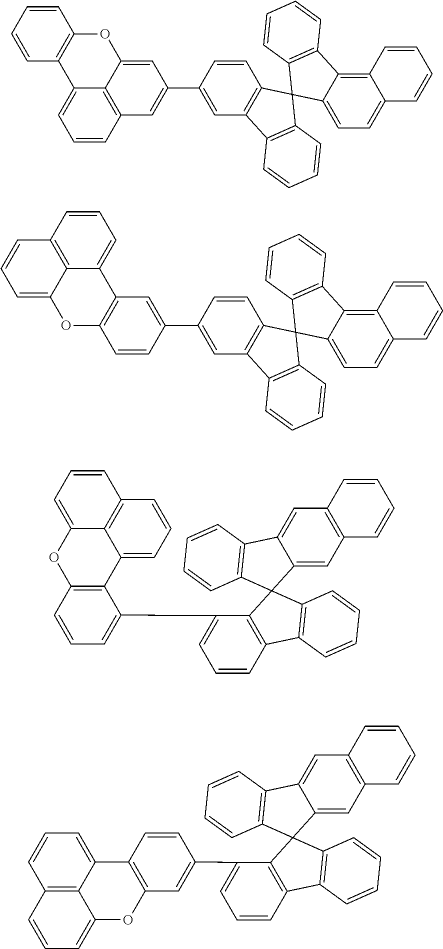 [SMILES: c1ccc2c(c1)Oc1cc(-c3ccc4c(c3)-c3ccccc3C43c4ccccc4-c4c3ccc3ccccc43)cc3cccc-2c13 ; c1ccc2c(c1)-c1cc(-c3ccc4c(c3)-c3cccc5cccc(c35)O4)ccc1C21c2ccccc2-c2c1ccc1ccccc21 ; c1ccc2c(c1)-c1cc3ccccc3cc1C21c2ccccc2-c2cccc(-c3cccc4c3-c3cccc5cccc(c35)O4)c21 ; c1ccc2c(c1)-c1cc3ccccc3cc1C21c2ccccc2-c2cccc(-c3ccc4c(c3)Oc3cccc5cccc-4c35)c21]

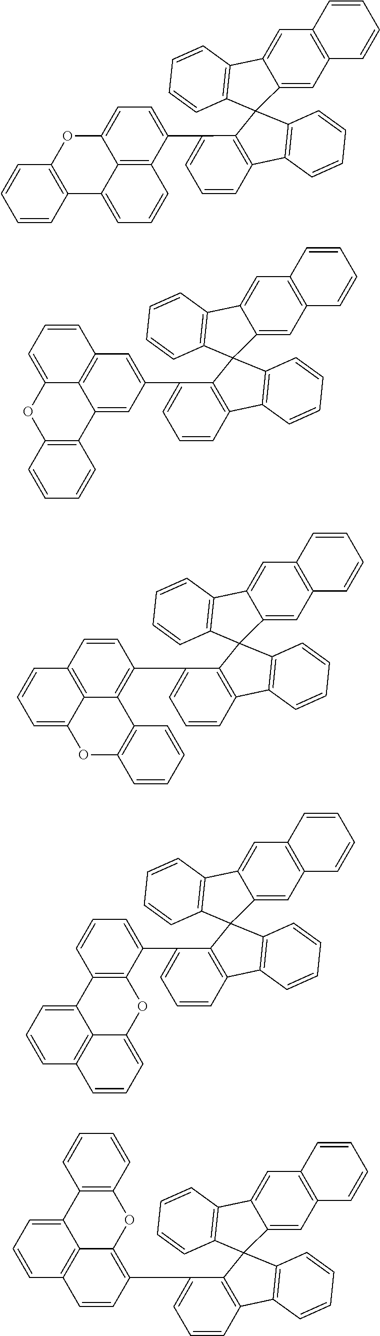

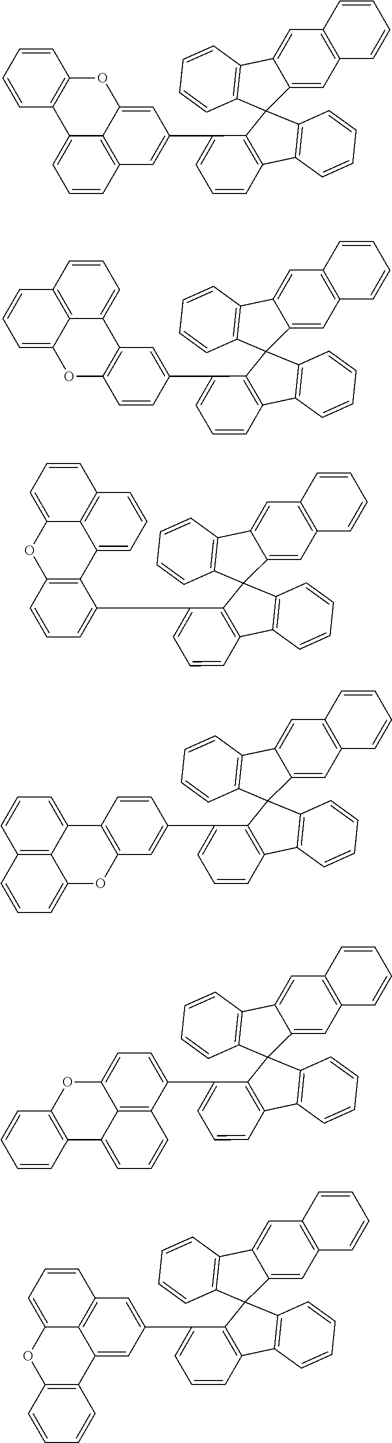 [SMILES: c1ccc2c(c1)Oc1cc(-c3cccc4c3C3(c5ccccc5-c5cc6ccccc6cc53)c3ccccc3-4)cc3cccc-2c13 ; c1ccc2c(c1)-c1cc3ccccc3cc1C21c2ccccc2-c2cccc(-c3ccc4c(c3)-c3cccc5cccc(c35)O4)c21 ; c1ccc2c(c1)-c1cc3ccccc3cc1C21c2ccccc2-c2cccc(-c3cccc4c3-c3cccc5cccc(c35)O4)c21 ; c1ccc2c(c1)-c1cc3ccccc3cc1C21c2ccccc2-c2cccc(-c3ccc4c(c3)Oc3cccc5cccc-4c35)c21 ; c1ccc2c(c1)Oc1ccc(-c3cccc4c3C3(c5ccccc5-c5cc6ccccc6cc53)c3ccccc3-4)c3cccc-2c13 ; c1ccc2c(c1)Oc1cccc3cc(-c4cccc5c4C4(c6ccccc6-c6cc7ccccc7cc64)c4ccccc4-5)cc-2c13]

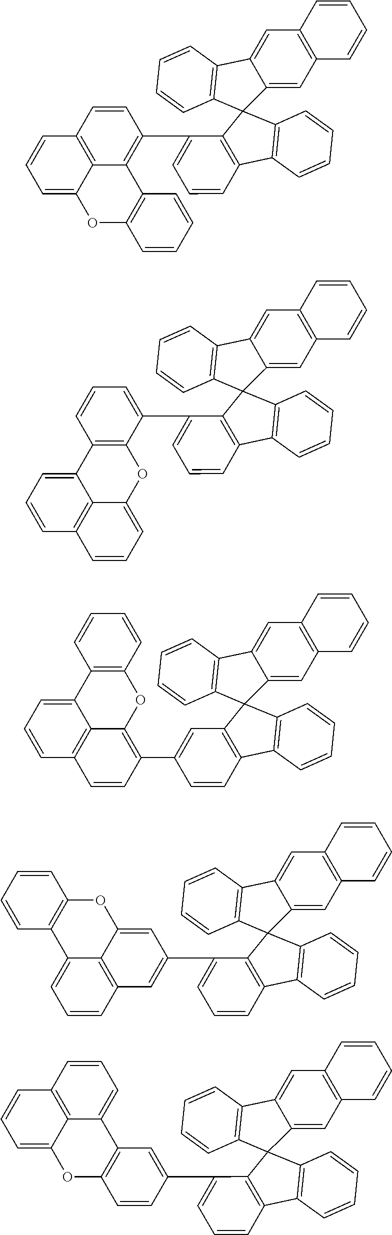

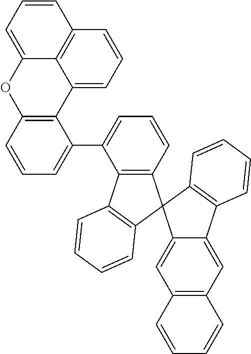 [SMILES: c1ccc2c(c1)-c1cc3ccccc3cc1C21c2ccccc2-c2c(-c3cccc4c3-c3cccc5cccc(c35)O4)cccc21]

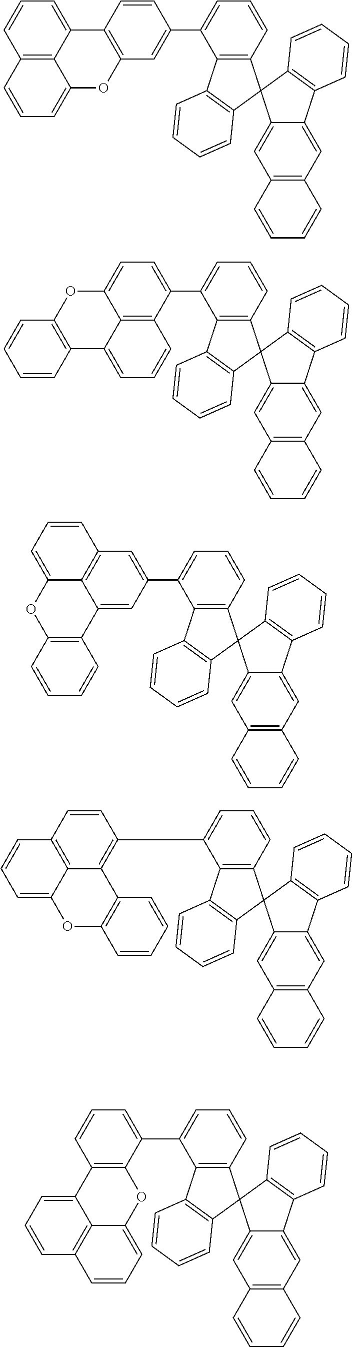

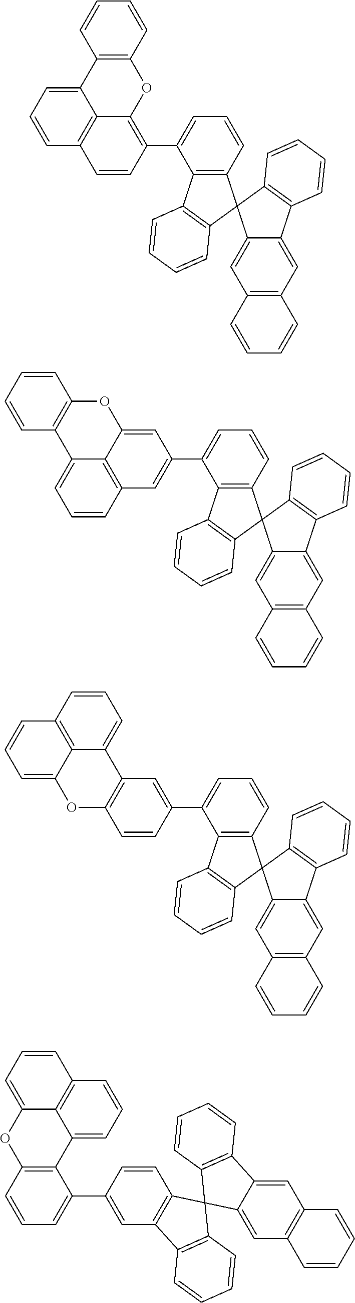 [SMILES: c1ccc2c(c1)Oc1c(-c3cccc4c3-c3ccccc3C43c4ccccc4-c4cc5ccccc5cc43)ccc3cccc-2c13 ; c1ccc2c(c1)Oc1cc(-c3cccc4c3-c3ccccc3C43c4ccccc4-c4cc5ccccc5cc43)cc3cccc-2c13 ; c1ccc2c(c1)-c1cc3ccccc3cc1C21c2ccccc2-c2c(-c3ccc4c(c3)-c3cccc5cccc(c35)O4)cccc21 ; c1ccc2c(c1)-c1cc(-c3cccc4c3-c3cccc5cccc(c35)O4)ccc1C21c2ccccc2-c2cc3ccccc3cc21]

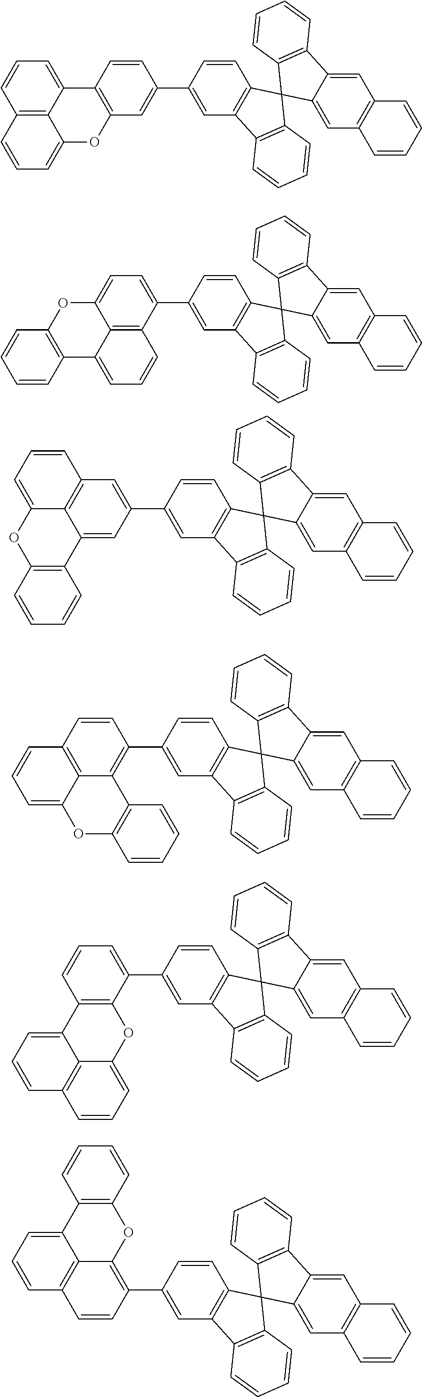

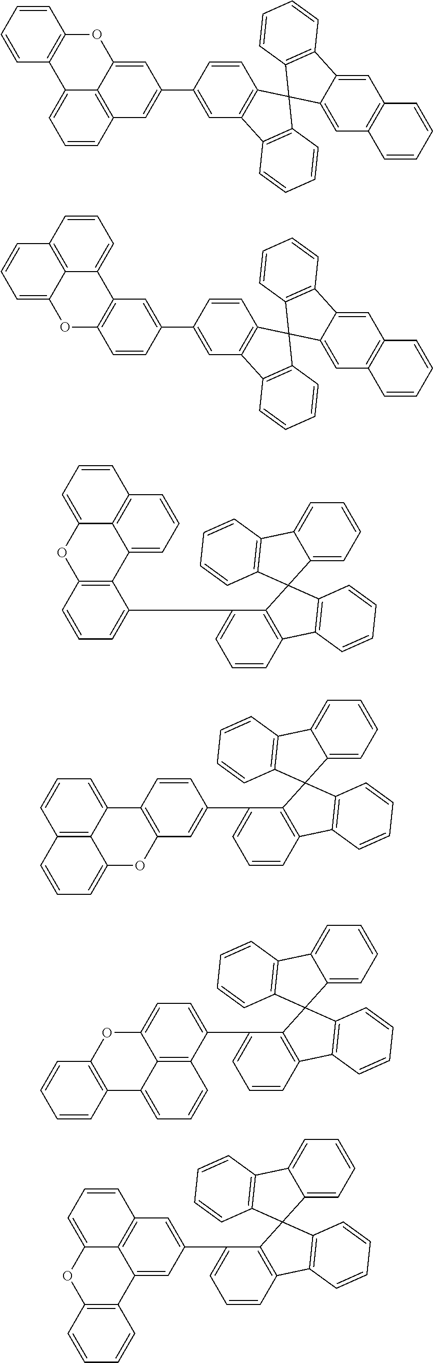

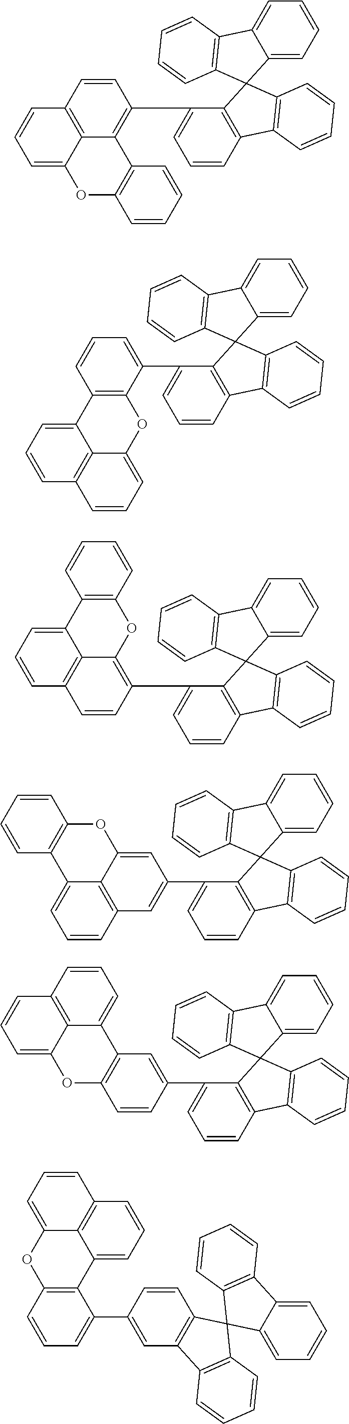

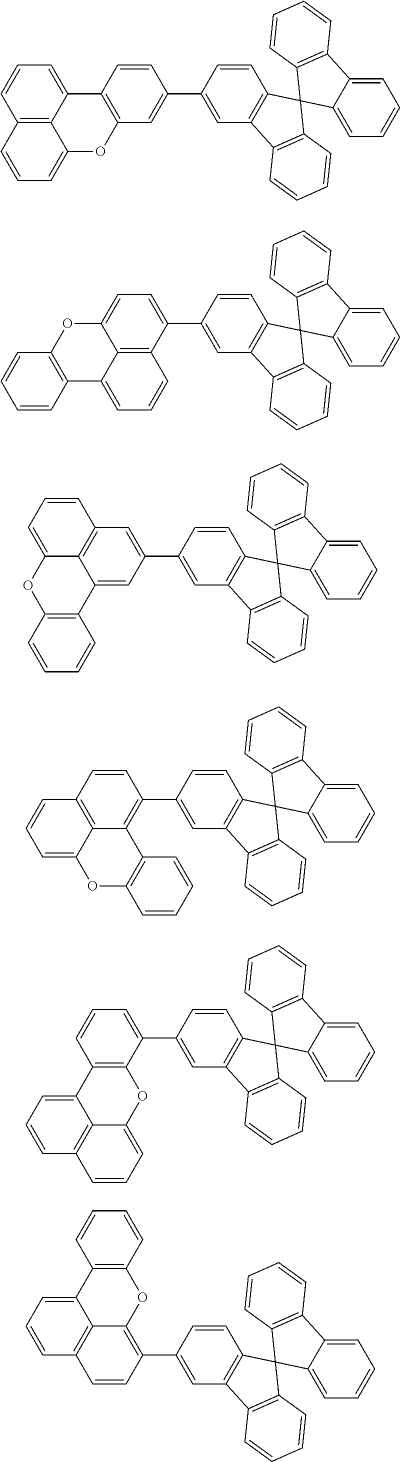

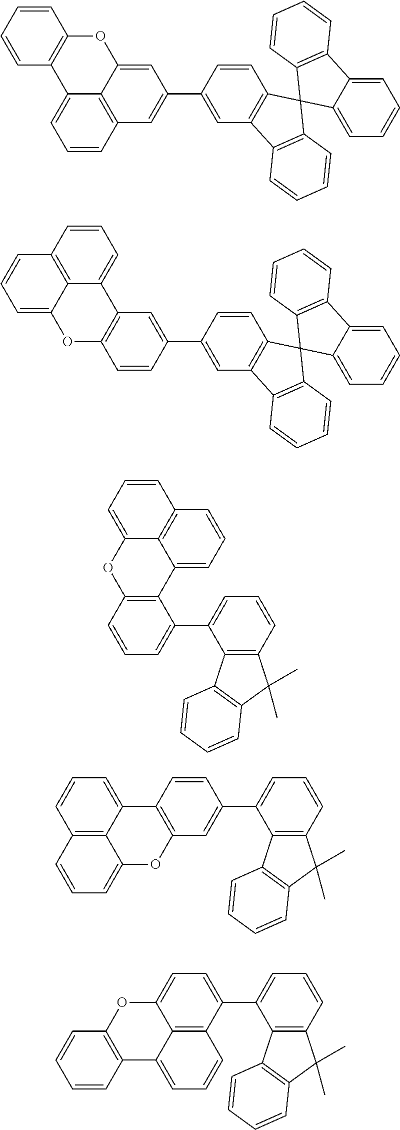

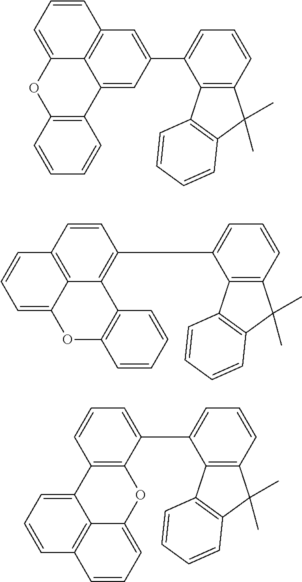

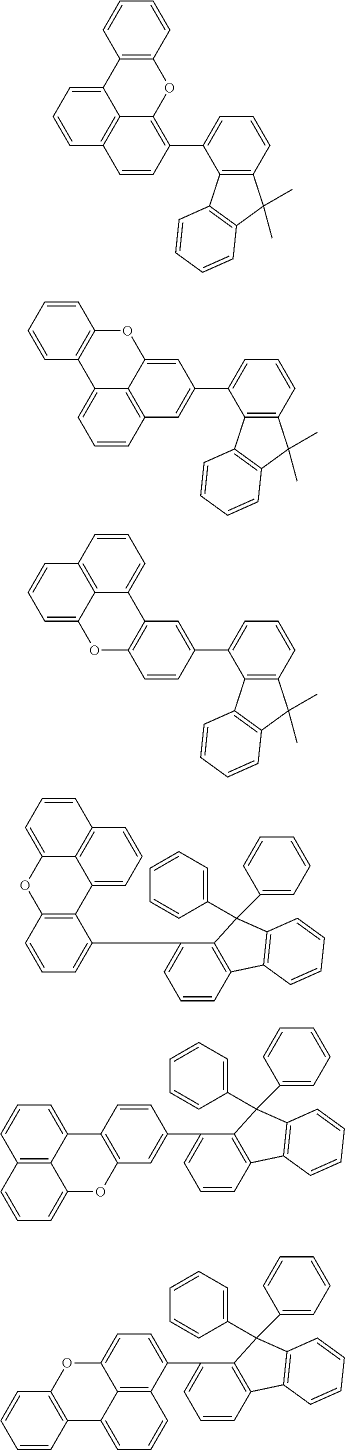

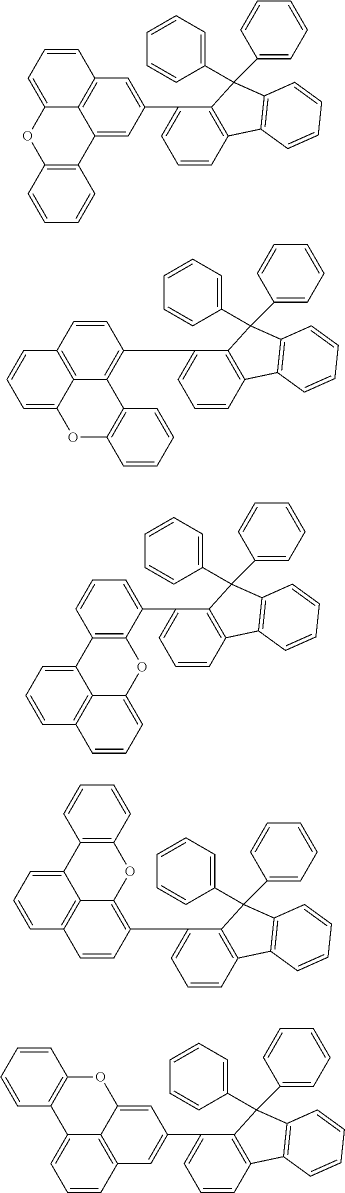

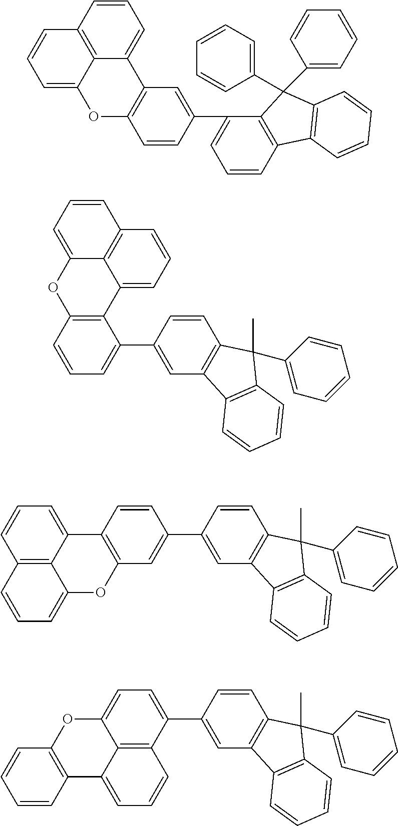 [SMILES: c1ccc(C2(c3ccccc3)c3ccccc3-c3cccc(-c4ccc5c(c4)-c4cccc6cccc(c46)O5)c32)cc1 ; CC1(c2ccccc2)c2ccccc2-c2cc(-c3cccc4c3-c3cccc5cccc(c35)O4)ccc21 ; CC1(c2ccccc2)c2ccccc2-c2cc(-c3ccc4c(c3)Oc3cccc5cccc-4c35)ccc21 ; CC1(c2ccccc2)c2ccccc2-c2cc(-c3ccc4c5c(cccc35)-c3ccccc3O4)ccc21]

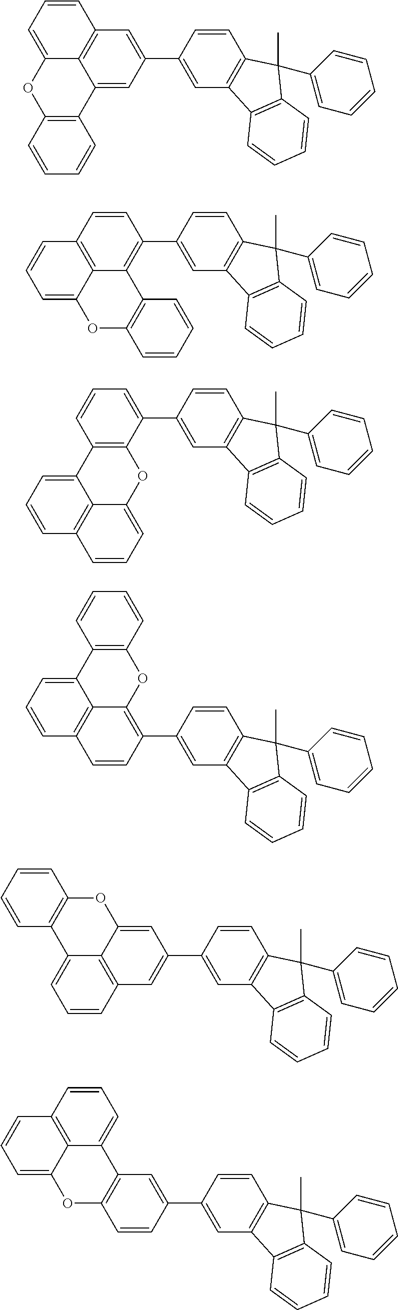

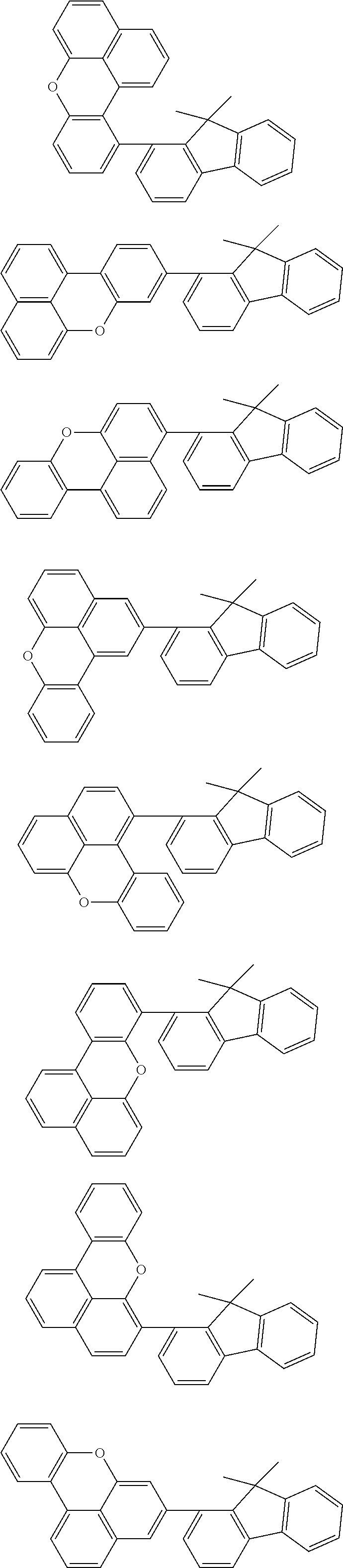

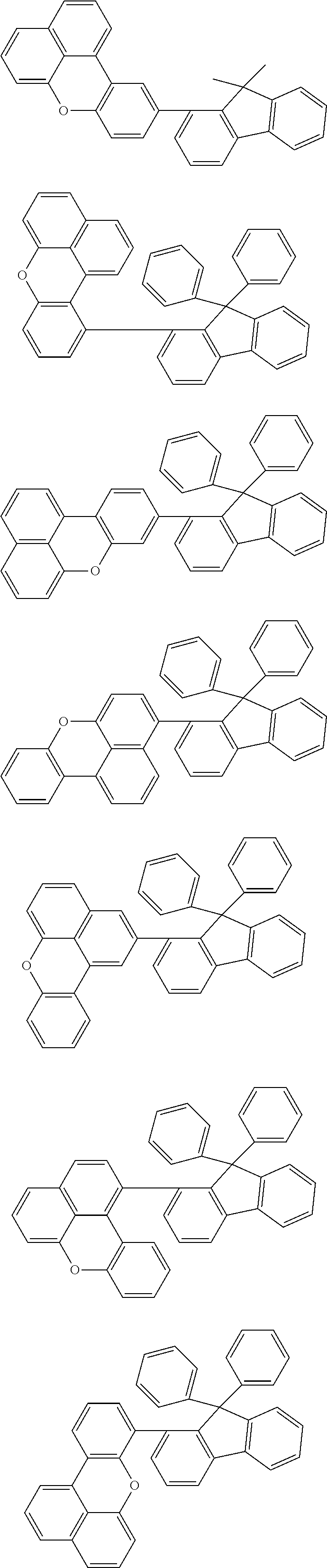

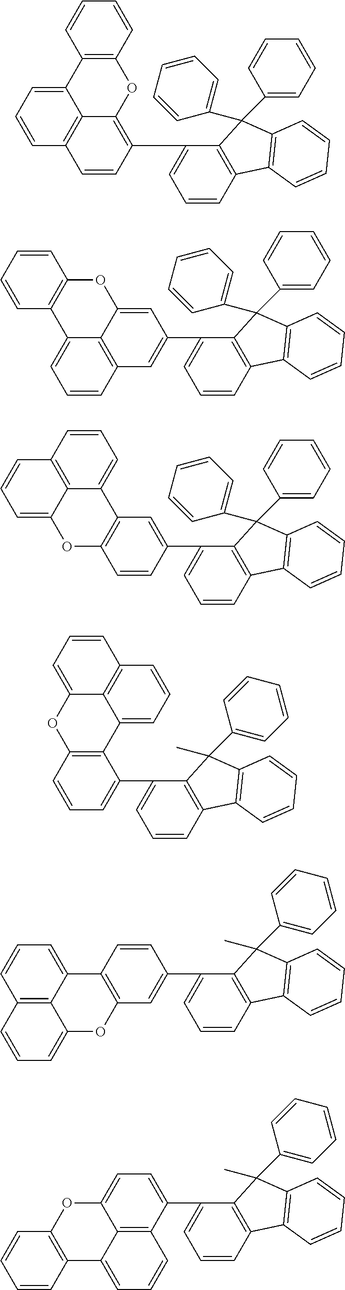

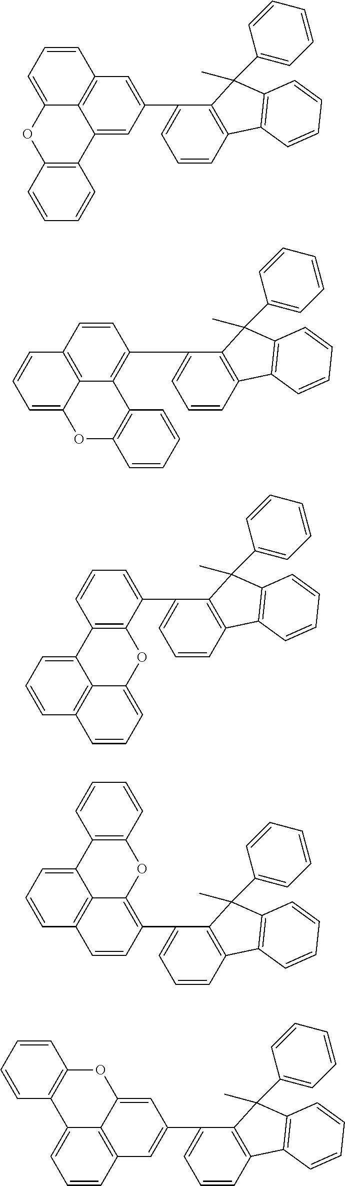

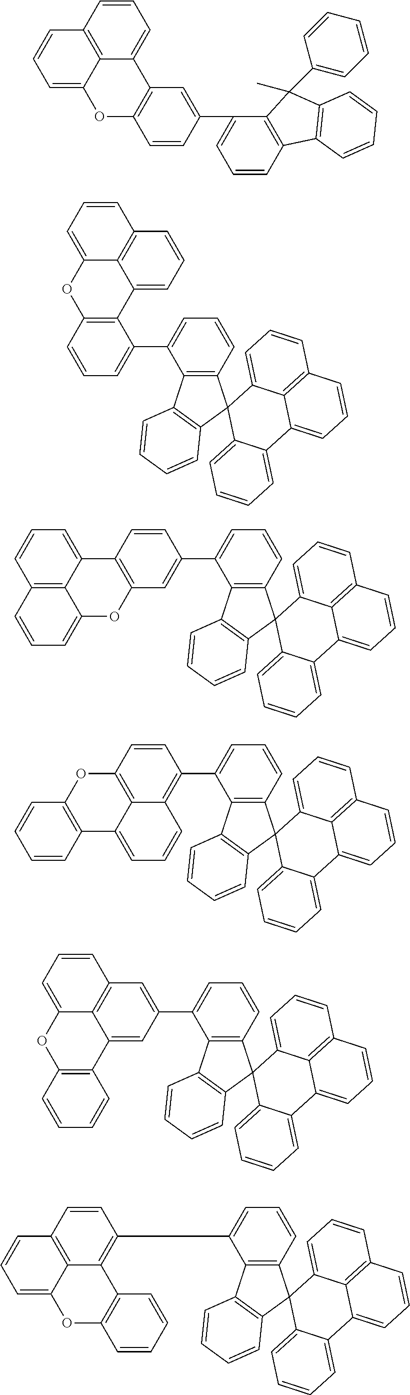

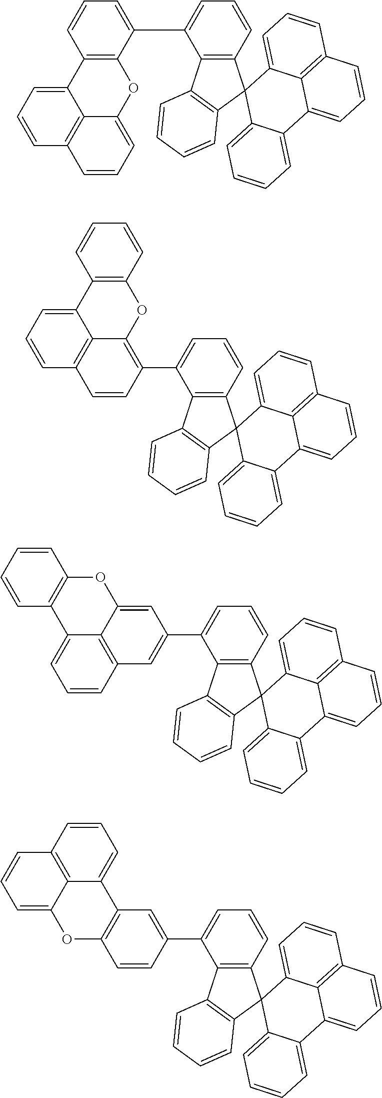 [SMILES: c1ccc2c(c1)-c1c(-c3cccc4c3Oc3cccc5cccc-4c35)cccc1C21c2ccccc2-c2cccc3cccc1c23 ; c1ccc2c(c1)Oc1c(-c3cccc4c3-c3ccccc3C43c4ccccc4-c4cccc5cccc3c45)ccc3cccc-2c13 ; c1ccc2c(c1)Oc1cc(-c3cccc4c3-c3ccccc3C43c4ccccc4-c4cccc5cccc3c45)cc3cccc-2c13 ; c1ccc2c(c1)-c1c(-c3ccc4c(c3)-c3cccc5cccc(c35)O4)cccc1C21c2ccccc2-c2cccc3cccc1c23]

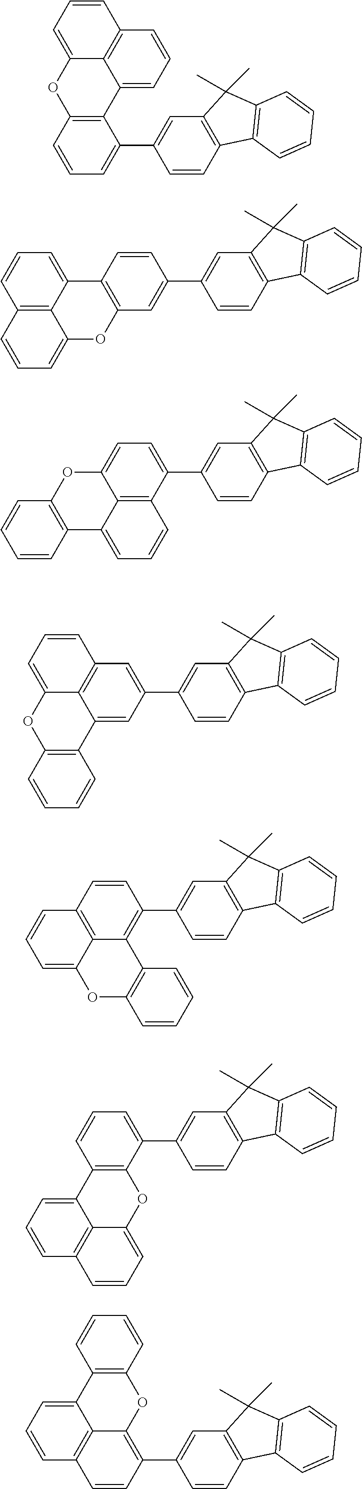

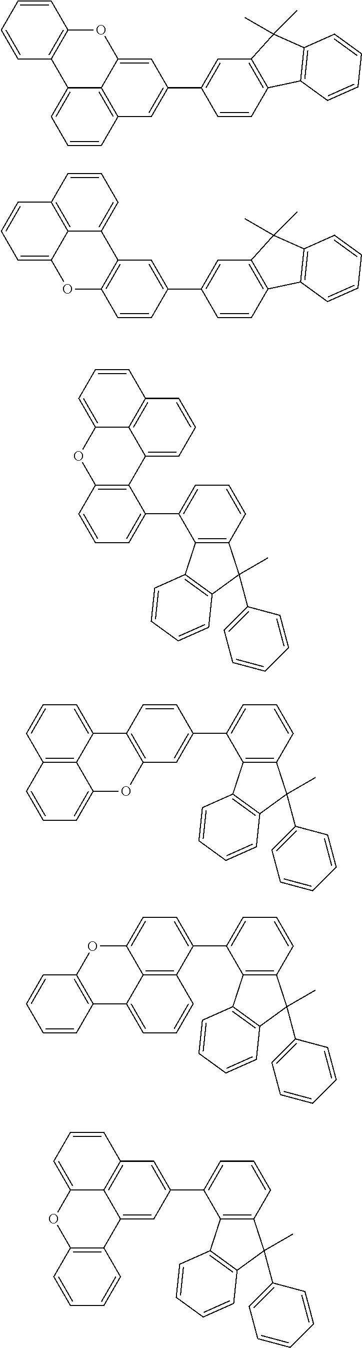

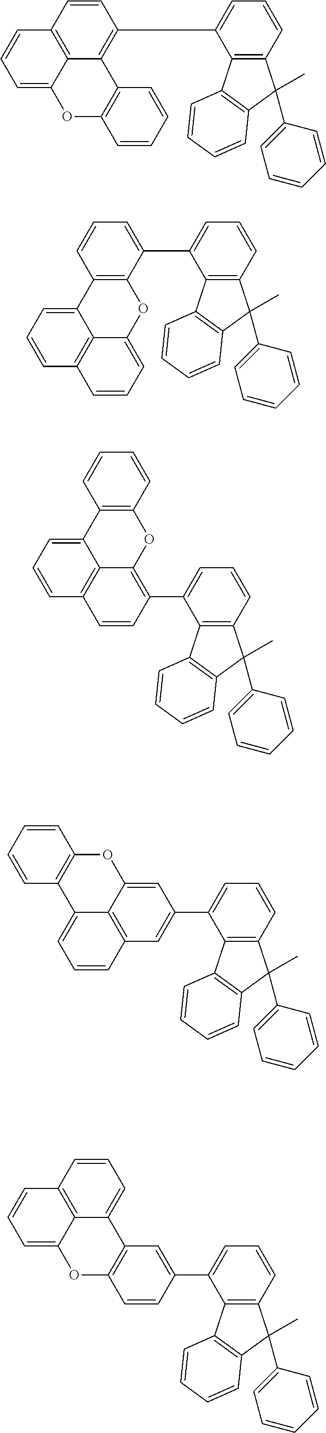

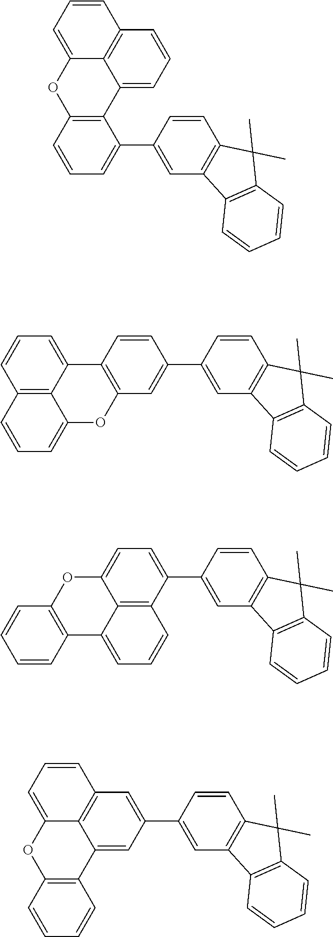 [SMILES: CC1(C)c2ccccc2-c2cc(-c3cccc4c3-c3cccc5cccc(c35)O4)ccc21 ; CC1(C)c2ccccc2-c2cc(-c3ccc4c(c3)Oc3cccc5cccc-4c35)ccc21 ; CC1(C)c2ccccc2-c2cc(-c3ccc4c5c(cccc35)-c3ccccc3O4)ccc21 ; CC1(C)c2ccccc2-c2cc(-c3cc4c5c(cccc5c3)Oc3ccccc3-4)ccc21]

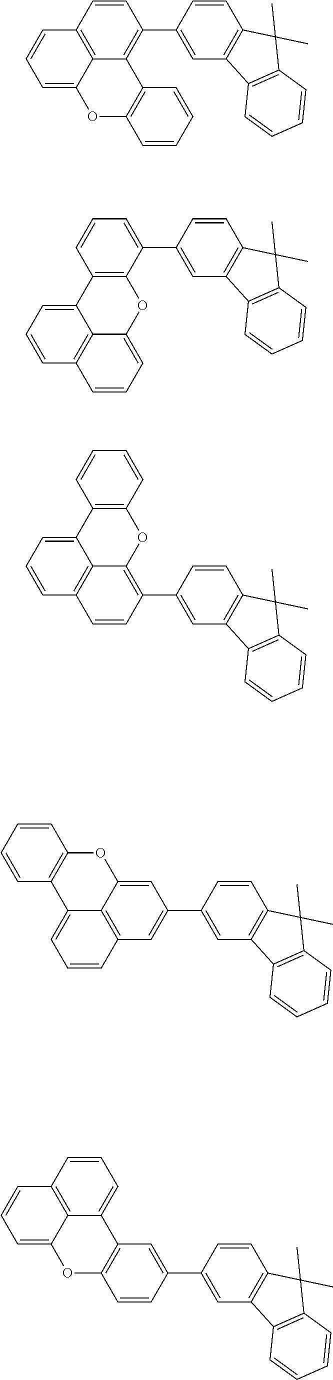 [SMILES: CC1(C)c2ccccc2-c2cc(-c3ccc4cccc5c4c3-c3ccccc3O5)ccc21 ; CC1(C)c2ccccc2-c2cc(-c3cccc4c3Oc3cccc5cccc-4c35)ccc21 ; CC1(C)c2ccccc2-c2cc(-c3ccc4cccc5c4c3Oc3ccccc3-5)ccc21 ; CC1(C)c2ccccc2-c2cc(-c3cc4c5c(cccc5c3)-c3ccccc3O4)ccc21 ; CC1(C)c2ccccc2-c2cc(-c3ccc4c(c3)-c3cccc5cccc(c35)O4)ccc21]

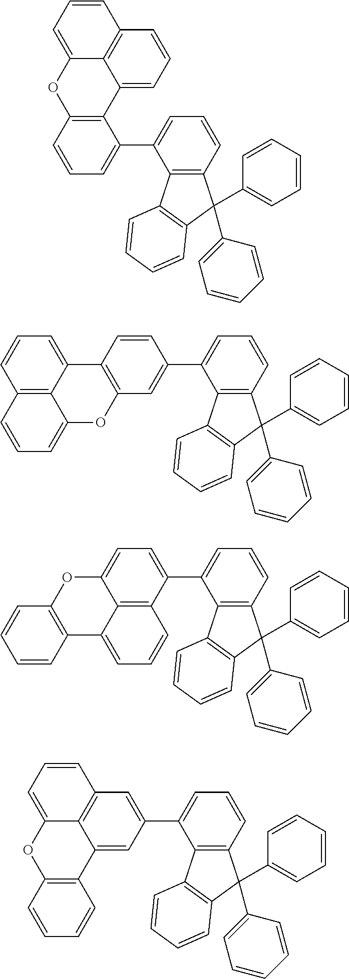

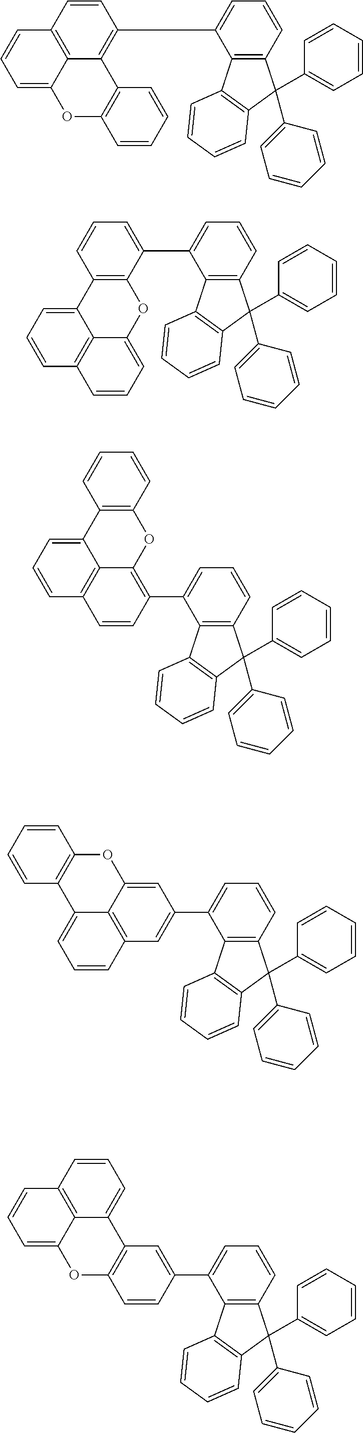

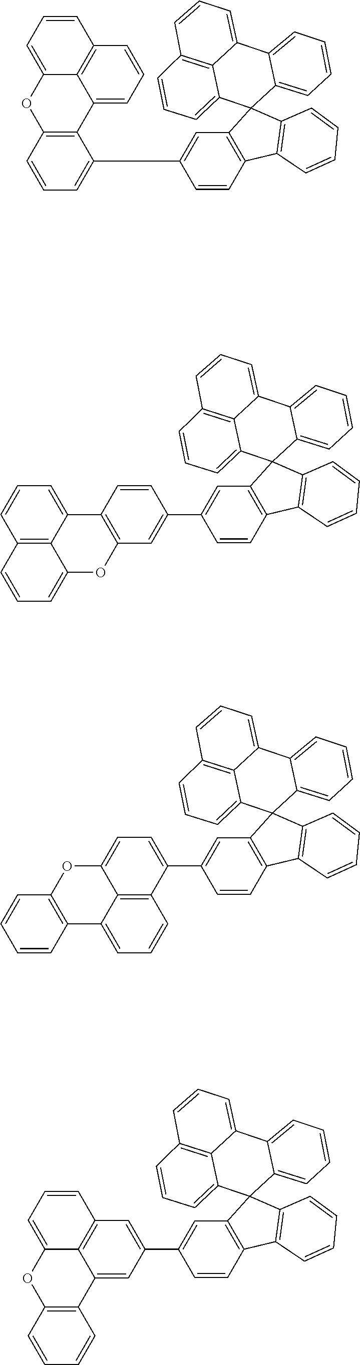 [SMILES: c1ccc2c(c1)-c1ccc(-c3cccc4c3-c3cccc5cccc(c35)O4)cc1C21c2ccccc2-c2cccc3cccc1c23 ; c1ccc2c(c1)-c1ccc(-c3ccc4c(c3)Oc3cccc5cccc-4c35)cc1C21c2ccccc2-c2cccc3cccc1c23 ; c1ccc2c(c1)Oc1ccc(-c3ccc4c(c3)C3(c5ccccc5-4)c4ccccc4-c4cccc5cccc3c45)c3cccc-2c13 ; c1ccc2c(c1)Oc1cccc3cc(-c4ccc5c(c4)C4(c6ccccc6-5)c5ccccc5-c5cccc6cccc4c56)cc-2c13]

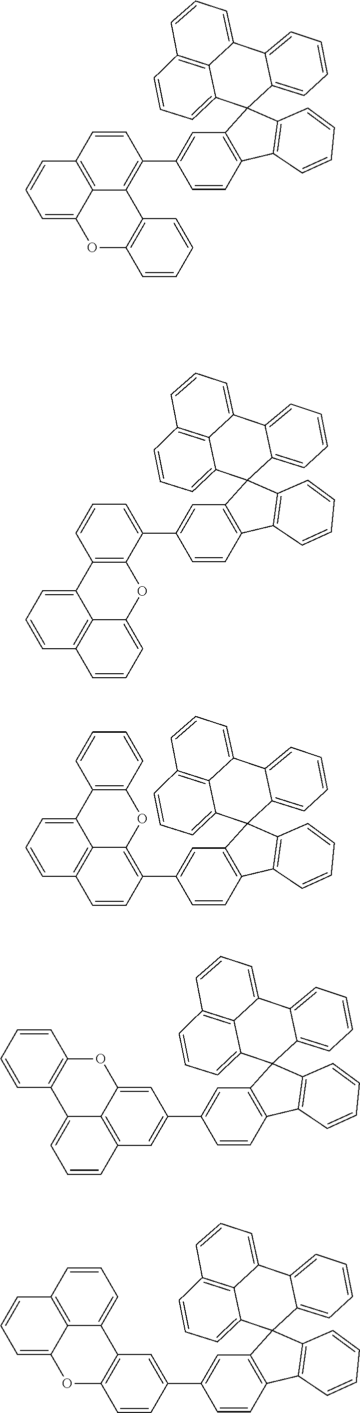 [SMILES: c1ccc2c(c1)Oc1cccc3ccc(-c4ccc5c(c4)C4(c6ccccc6-5)c5ccccc5-c5cccc6cccc4c56)c-2c13 ; c1ccc2c(c1)-c1ccc(-c3cccc4c3Oc3cccc5cccc-4c35)cc1C21c2ccccc2-c2cccc3cccc1c23 ; c1ccc2c(c1)Oc1c(-c3ccc4c(c3)C3(c5ccccc5-4)c4ccccc4-c4cccc5cccc3c45)ccc3cccc-2c13 ; c1ccc2c(c1)Oc1cc(-c3ccc4c(c3)C3(c5ccccc5-4)c4ccccc4-c4cccc5cccc3c45)cc3cccc-2c13 ; c1ccc2c(c1)-c1ccc(-c3ccc4c(c3)-c3cccc5cccc(c35)O4)cc1C21c2ccccc2-c2cccc3cccc1c23]

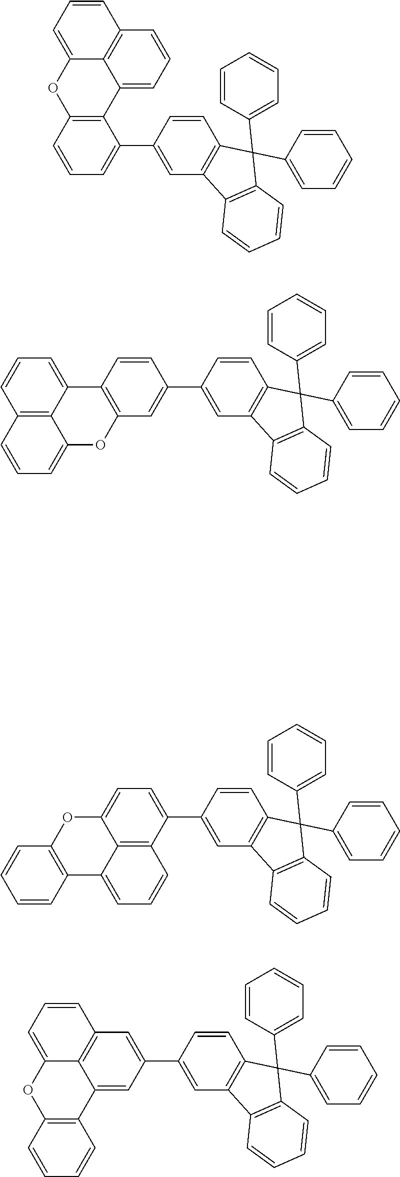 [SMILES: c1ccc(C2(c3ccccc3)c3ccccc3-c3cc(-c4cccc5c4-c4cccc6cccc(c46)O5)ccc32)cc1 ; c1ccc(C2(c3ccccc3)c3ccccc3-c3cc(-c4ccc5c(c4)Oc4cccc6cccc-5c46)ccc32)cc1 ; c1ccc(C2(c3ccccc3)c3ccccc3-c3cc(-c4ccc5c6c(cccc46)-c4ccccc4O5)ccc32)cc1 ; c1ccc(C2(c3ccccc3)c3ccccc3-c3cc(-c4cc5c6c(cccc6c4)Oc4ccccc4-5)ccc32)cc1]

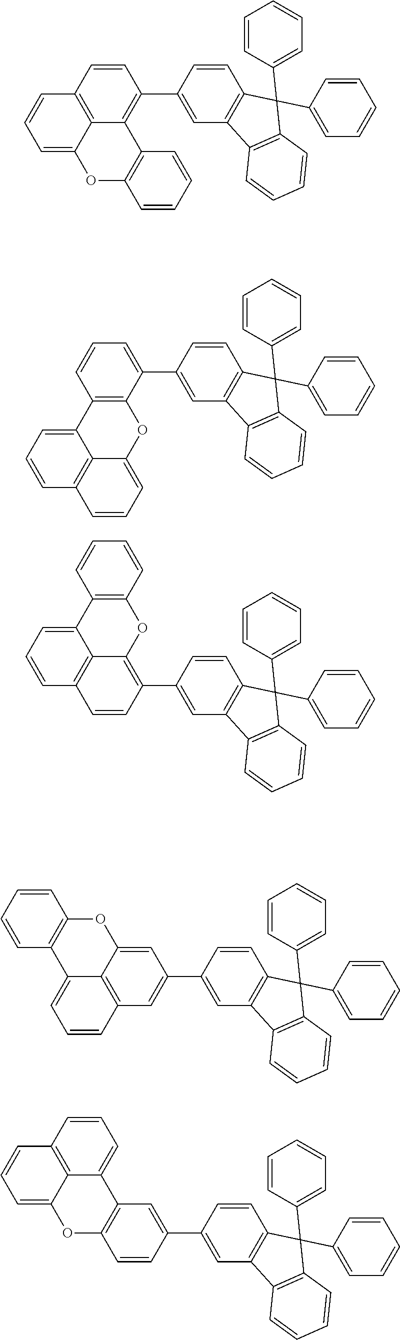

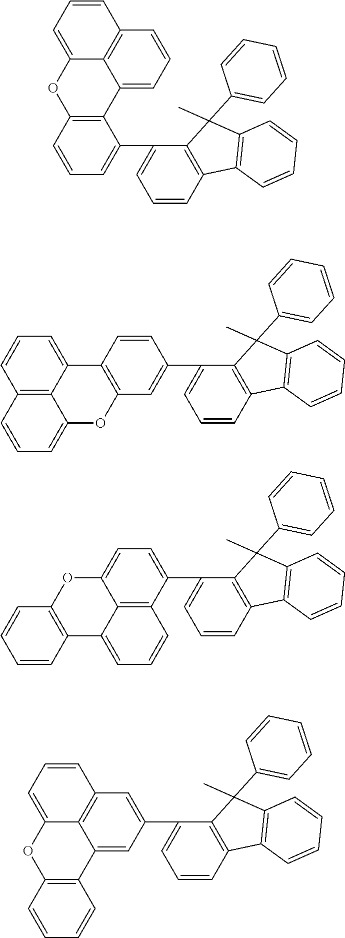 [SMILES: CC1(c2ccccc2)c2ccccc2-c2cccc(-c3cccc4c3-c3cccc5cccc(c35)O4)c21 ; CC1(c2ccccc2)c2ccccc2-c2cccc(-c3ccc4c(c3)Oc3cccc5cccc-4c35)c21 ; CC1(c2ccccc2)c2ccccc2-c2cccc(-c3ccc4c5c(cccc35)-c3ccccc3O4)c21 ; CC1(c2ccccc2)c2ccccc2-c2cccc(-c3cc4c5c(cccc5c3)Oc3ccccc3-4)c21]

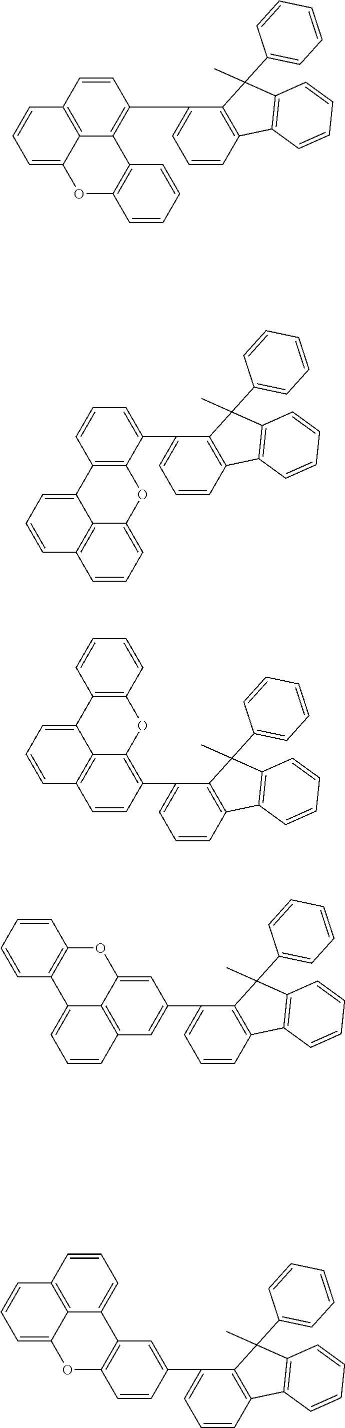

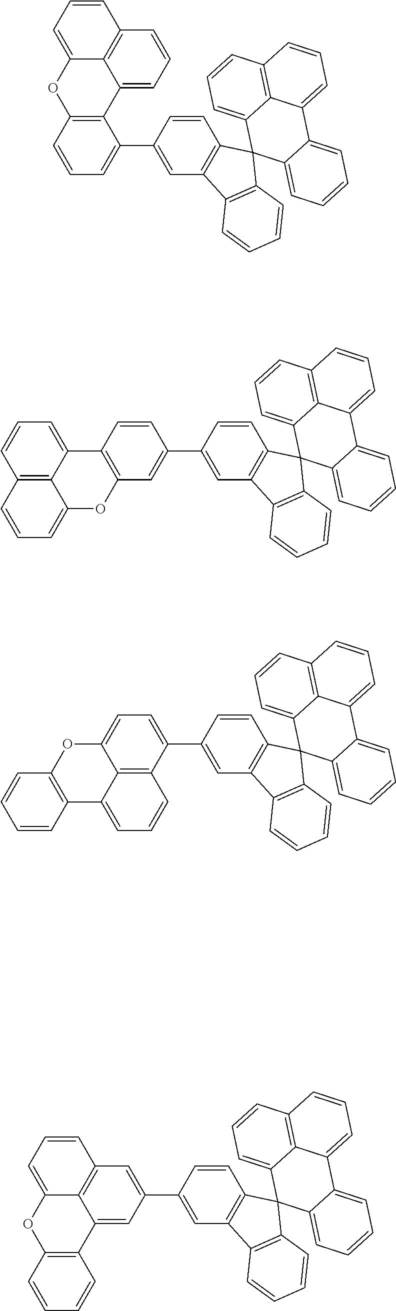 [SMILES: c1ccc2c(c1)-c1cc(-c3cccc4c3-c3cccc5cccc(c35)O4)ccc1C21c2ccccc2-c2cccc3cccc1c23 ; c1ccc2c(c1)-c1cc(-c3ccc4c(c3)Oc3cccc5cccc-4c35)ccc1C21c2ccccc2-c2cccc3cccc1c23 ; c1ccc2c(c1)Oc1ccc(-c3ccc4c(c3)-c3ccccc3C43c4ccccc4-c4cccc5cccc3c45)c3cccc-2c13 ; c1ccc2c(c1)Oc1cccc3cc(-c4ccc5c(c4)-c4ccccc4C54c5ccccc5-c5cccc6cccc4c56)cc-2c13]

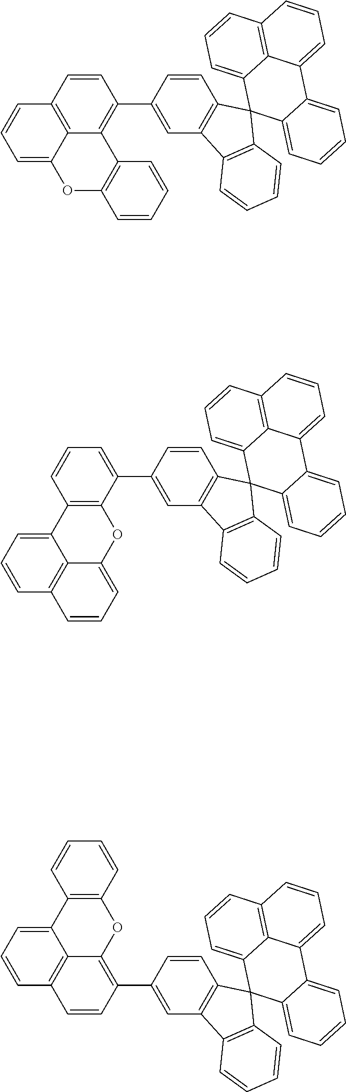

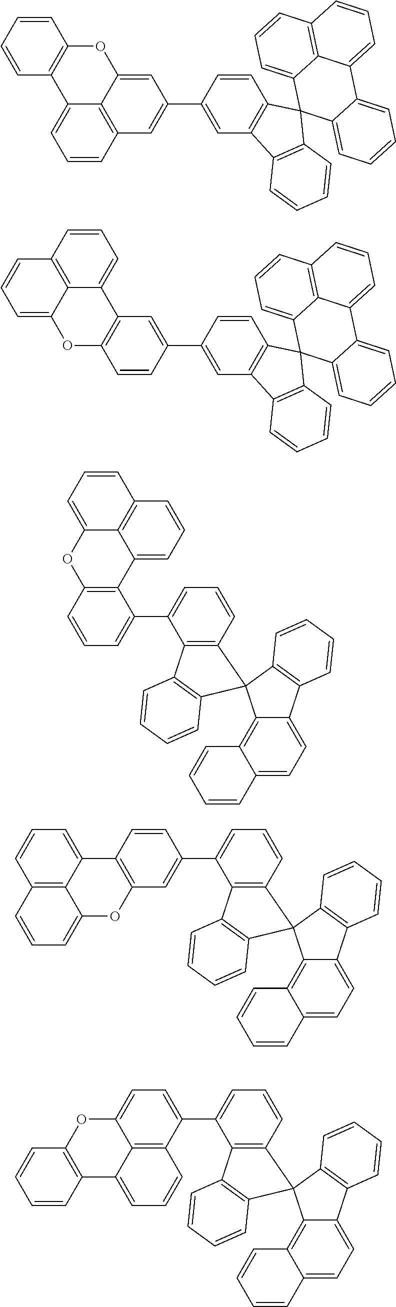

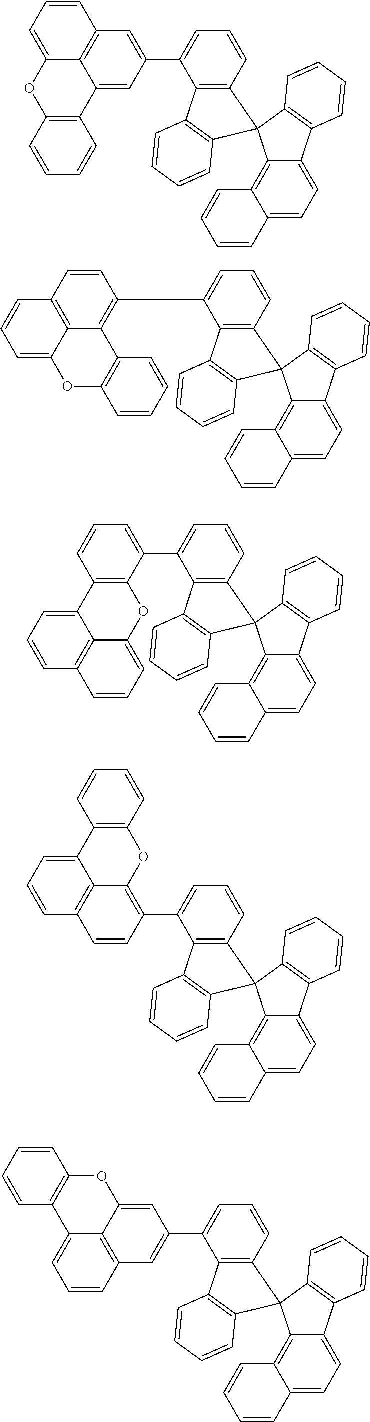

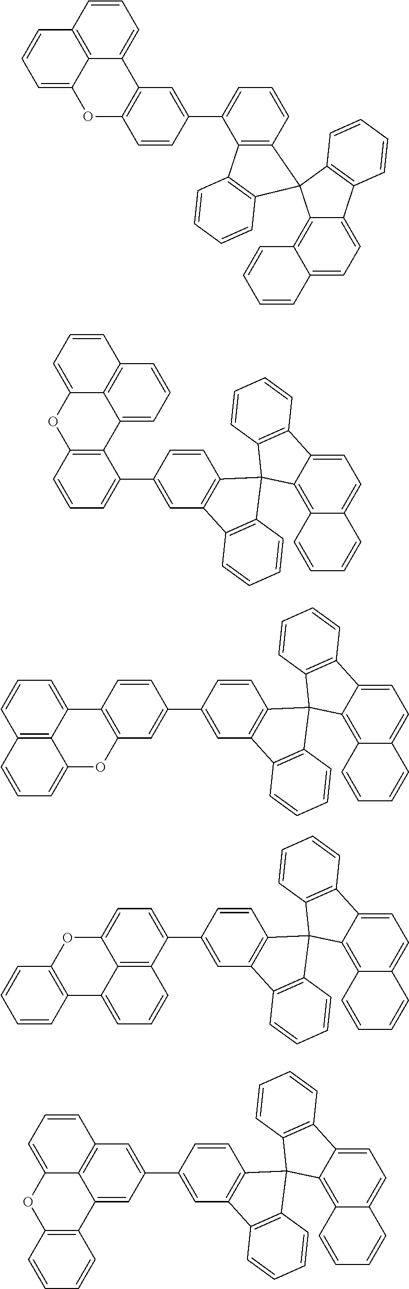

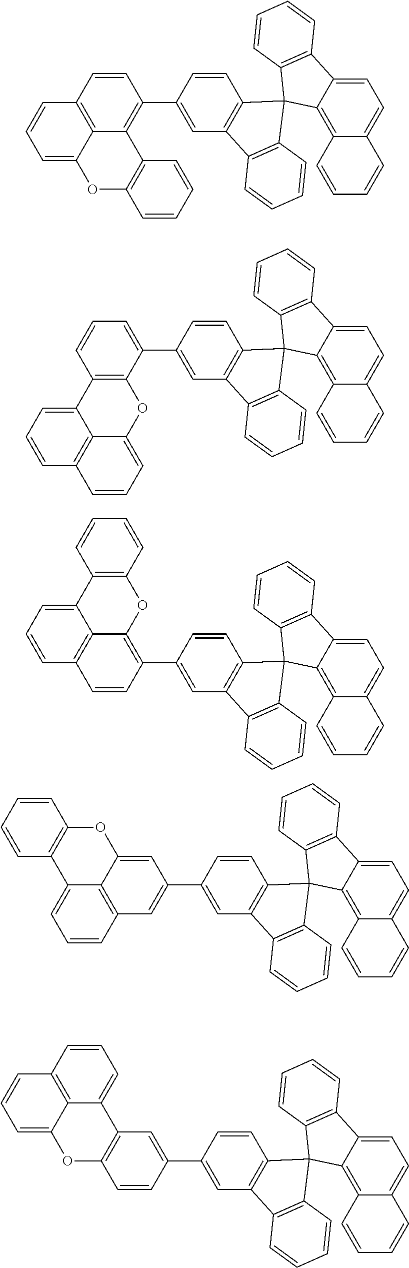

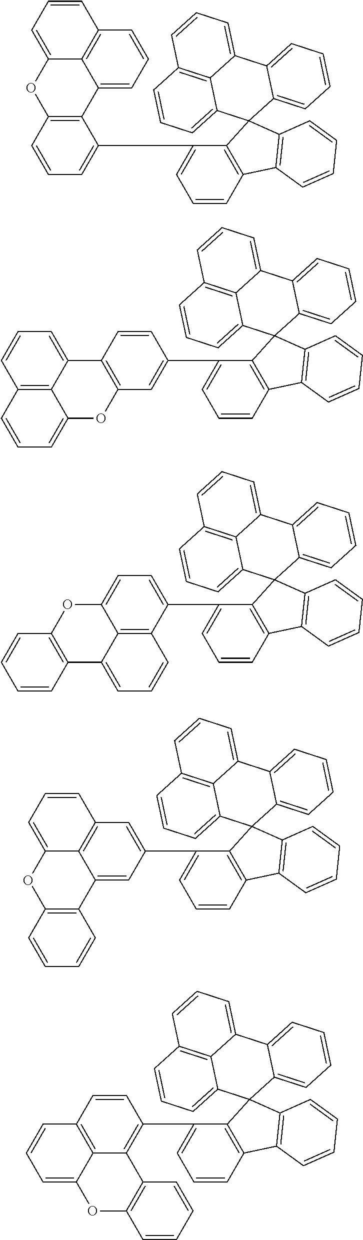

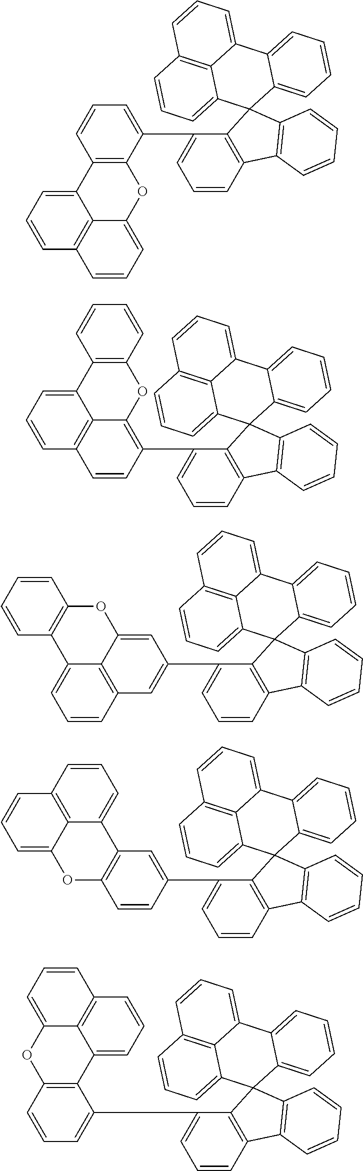

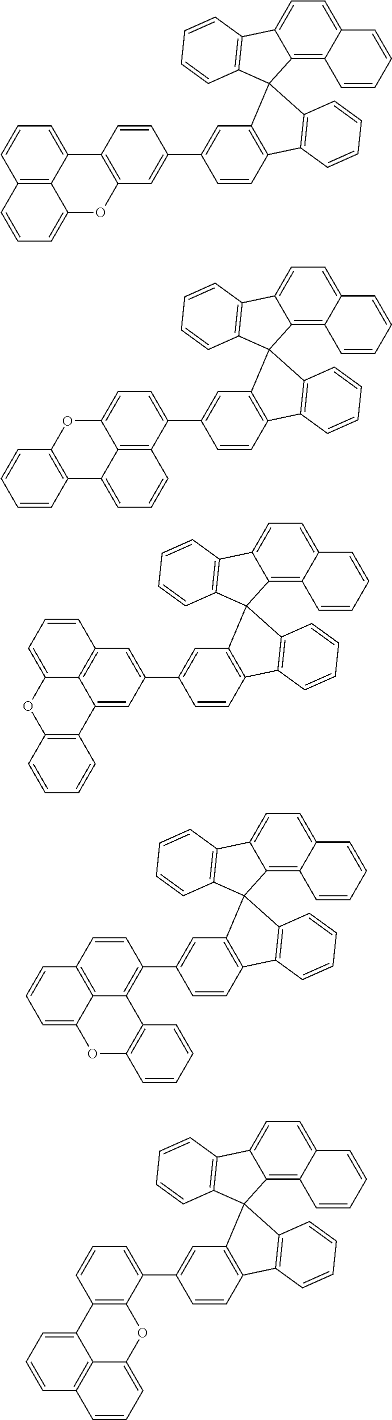 [SMILES: c1ccc2c(c1)-c1ccc(-c3ccc4c(c3)Oc3cccc5cccc-4c35)cc1C21c2ccccc2-c2ccc3ccccc3c21 ; c1ccc2c(c1)Oc1ccc(-c3ccc4c(c3)C3(c5ccccc5-4)c4ccccc4-c4ccc5ccccc5c43)c3cccc-2c13 ; c1ccc2c(c1)Oc1cccc3cc(-c4ccc5c(c4)C4(c6ccccc6-5)c5ccccc5-c5ccc6ccccc6c54)cc-2c13 ; c1ccc2c(c1)Oc1cccc3ccc(-c4ccc5c(c4)C4(c6ccccc6-5)c5ccccc5-c5ccc6ccccc6c54)c-2c13 ; c1ccc2c(c1)-c1ccc(-c3cccc4c3Oc3cccc5cccc-4c35)cc1C21c2ccccc2-c2ccc3ccccc3c21]

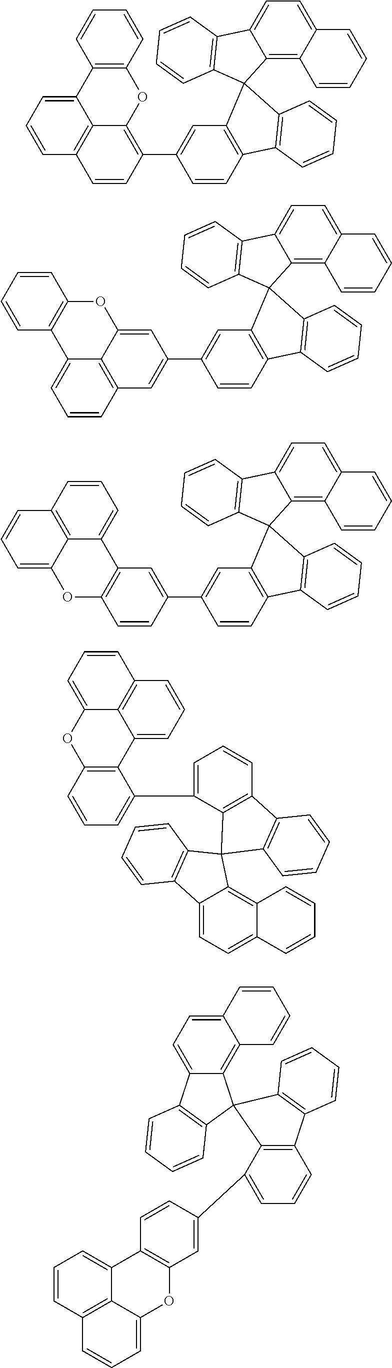

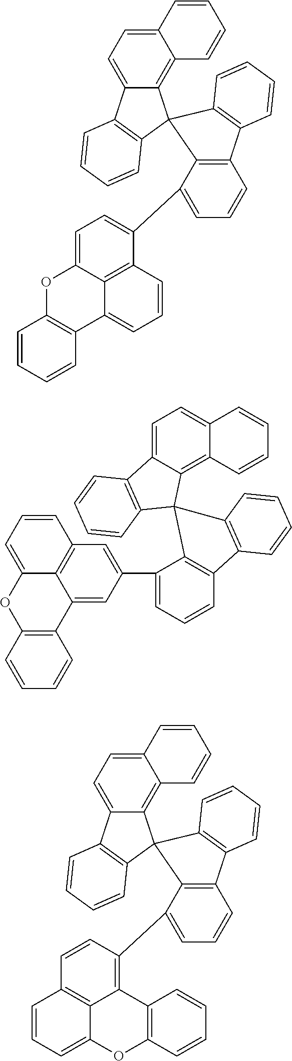 [SMILES: c1ccc2c(c1)Oc1ccc(-c3cccc4c3C3(c5ccccc5-4)c4ccccc4-c4ccc5ccccc5c43)c3cccc-2c13 ; c1ccc2c(c1)Oc1cccc3cc(-c4cccc5c4C4(c6ccccc6-5)c5ccccc5-c5ccc6ccccc6c54)cc-2c13 ; c1ccc2c(c1)Oc1cccc3ccc(-c4cccc5c4C4(c6ccccc6-5)c5ccccc5-c5ccc6ccccc6c54)c-2c13]

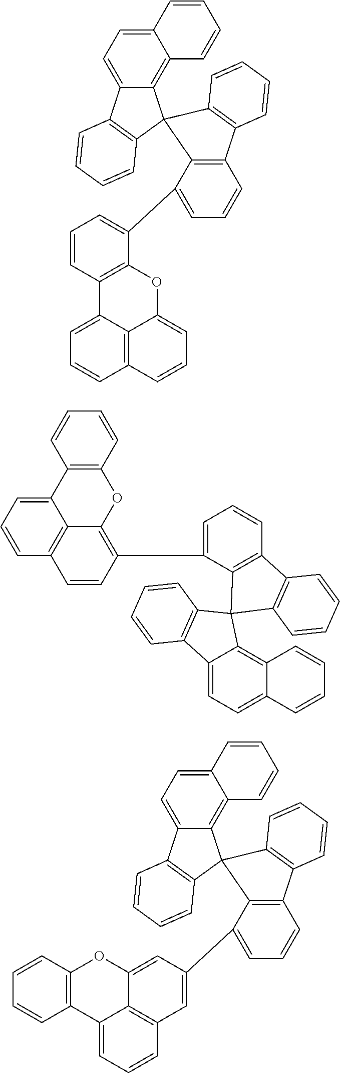

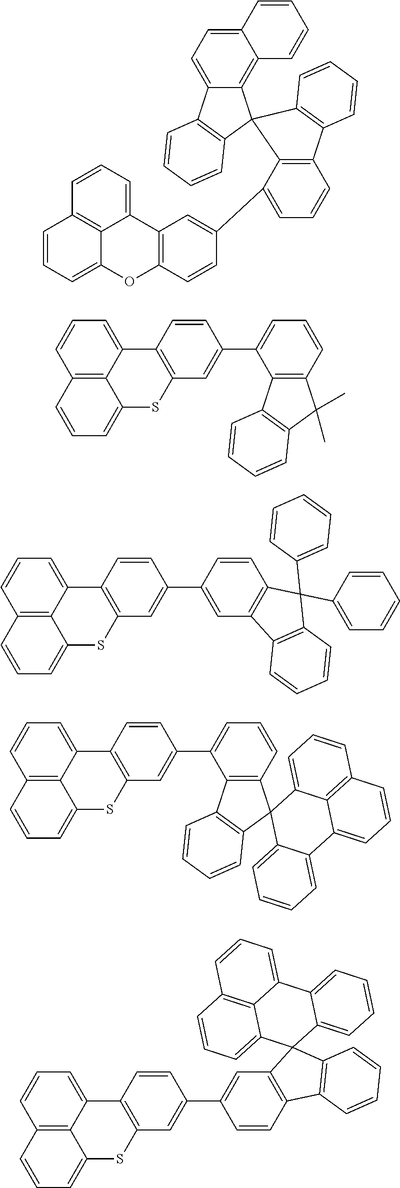

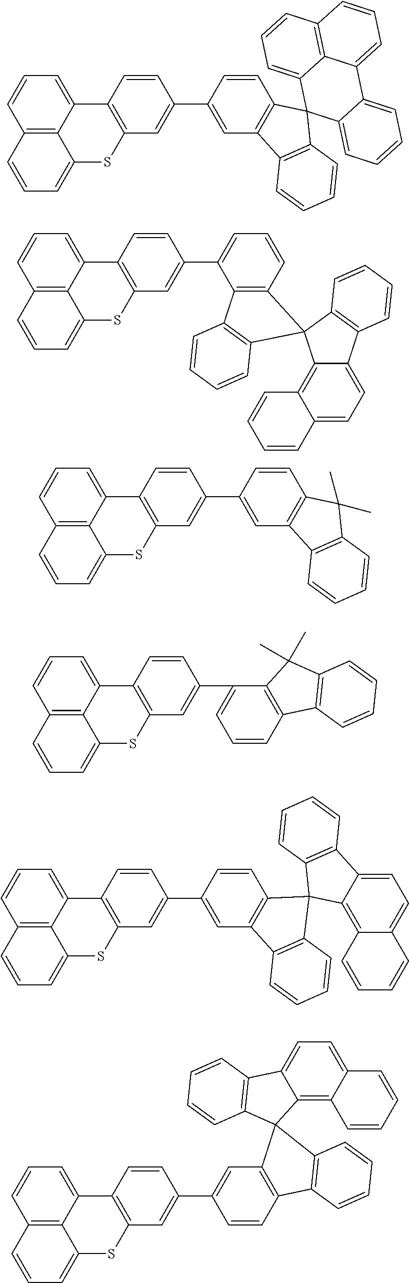

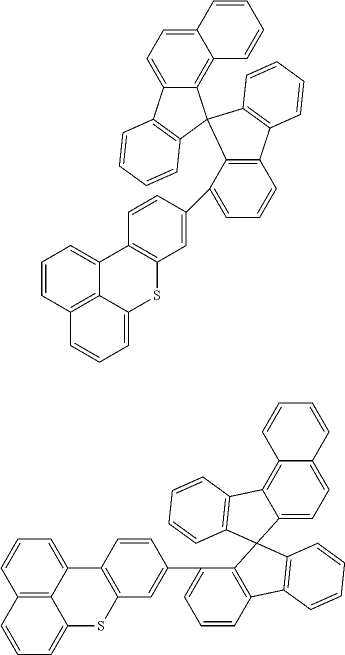 [SMILES: c1ccc2c(c1)-c1cccc(-c3ccc4c(c3)Sc3cccc5cccc-4c35)c1C21c2ccccc2-c2ccc3ccccc3c21 ; c1ccc2c(c1)-c1cccc(-c3ccc4c(c3)Sc3cccc5cccc-4c35)c1C21c2ccccc2-c2c1ccc1ccccc21]

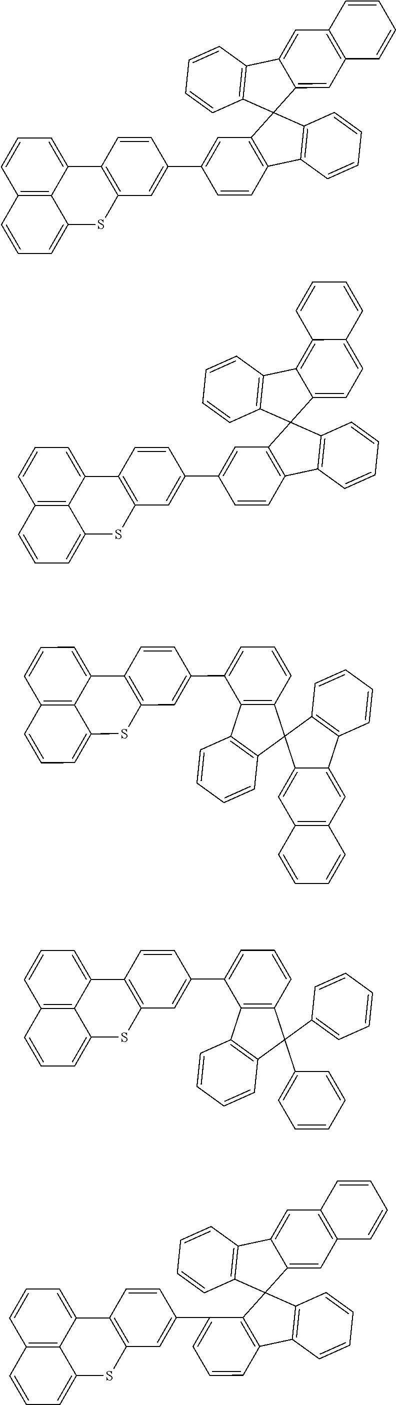 [SMILES: c1ccc2c(c1)-c1ccc(-c3ccc4c(c3)Sc3cccc5cccc-4c35)cc1C21c2ccccc2-c2cc3ccccc3cc21 ; c1ccc2c(c1)-c1ccc(-c3ccc4c(c3)Sc3cccc5cccc-4c35)cc1C21c2ccccc2-c2c1ccc1ccccc21 ; c1ccc2c(c1)-c1cc3ccccc3cc1C21c2ccccc2-c2c(-c3ccc4c(c3)Sc3cccc5cccc-4c35)cccc21 ; c1ccc(C2(c3ccccc3)c3ccccc3-c3c(-c4ccc5c(c4)Sc4cccc6cccc-5c46)cccc32)cc1 ; c1ccc2c(c1)-c1cc3ccccc3cc1C21c2ccccc2-c2cccc(-c3ccc4c(c3)Sc3cccc5cccc-4c35)c21]

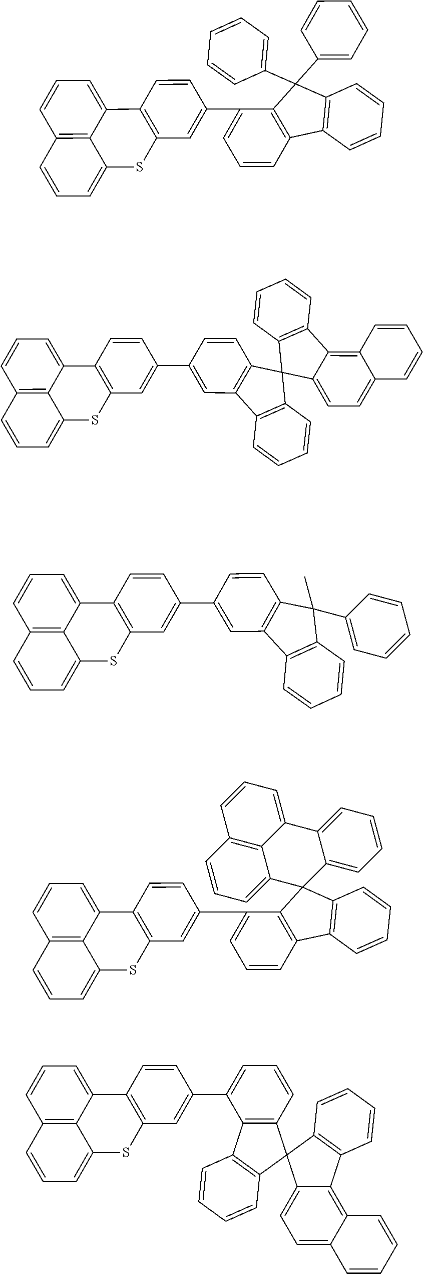 [SMILES: c1ccc(C2(c3ccccc3)c3ccccc3-c3cccc(-c4ccc5c(c4)Sc4cccc6cccc-5c46)c32)cc1 ; c1ccc2c(c1)-c1cc(-c3ccc4c(c3)Sc3cccc5cccc-4c35)ccc1C21c2ccccc2-c2c1ccc1ccccc21 ; CC1(c2ccccc2)c2ccccc2-c2cc(-c3ccc4c(c3)Sc3cccc5cccc-4c35)ccc21 ; c1ccc2c(c1)-c1cccc(-c3ccc4c(c3)Sc3cccc5cccc-4c35)c1C21c2ccccc2-c2cccc3cccc1c23 ; c1ccc2c(c1)-c1c(-c3ccc4c(c3)Sc3cccc5cccc-4c35)cccc1C21c2ccccc2-c2c1ccc1ccccc21]

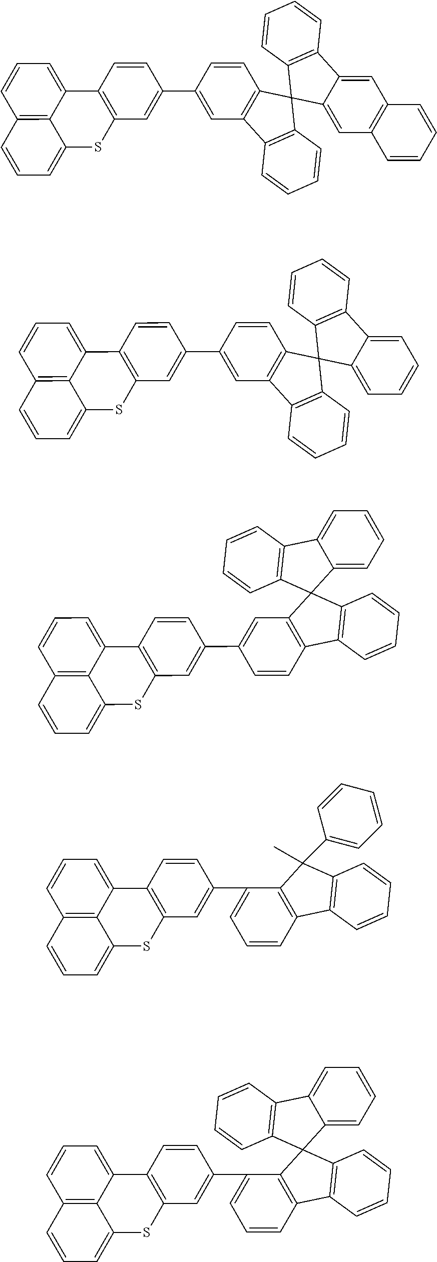 [SMILES: c1ccc2c(c1)-c1cc(-c3ccc4c(c3)Sc3cccc5cccc-4c35)ccc1C21c2ccccc2-c2cc3ccccc3cc21 ; c1ccc2c(c1)-c1ccccc1C21c2ccccc2-c2cc(-c3ccc4c(c3)Sc3cccc5cccc-4c35)ccc21 ; c1ccc2c(c1)-c1ccccc1C21c2ccccc2-c2ccc(-c3ccc4c(c3)Sc3cccc5cccc-4c35)cc21 ; CC1(c2ccccc2)c2ccccc2-c2cccc(-c3ccc4c(c3)Sc3cccc5cccc-4c35)c21 ; c1ccc2c(c1)-c1ccccc1C21c2ccccc2-c2cccc(-c3ccc4c(c3)Sc3cccc5cccc-4c35)c21]

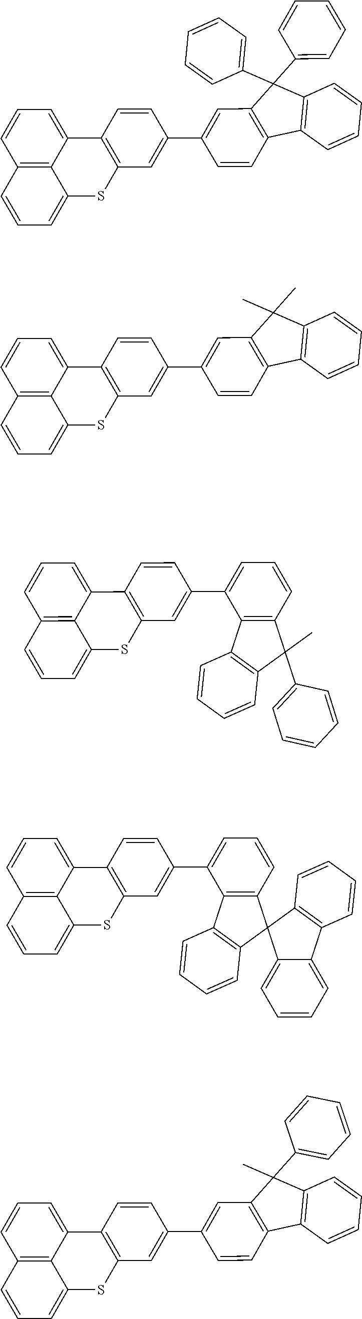 [SMILES: c1ccc(C2(c3ccccc3)c3ccccc3-c3ccc(-c4ccc5c(c4)Sc4cccc6cccc-5c46)cc32)cc1 ; CC1(C)c2ccccc2-c2ccc(-c3ccc4c(c3)Sc3cccc5cccc-4c35)cc21 ; CC1(c2ccccc2)c2ccccc2-c2c(-c3ccc4c(c3)Sc3cccc5cccc-4c35)cccc21 ; c1ccc2c(c1)-c1ccccc1C21c2ccccc2-c2c(-c3ccc4c(c3)Sc3cccc5cccc-4c35)cccc21 ; CC1(c2ccccc2)c2ccccc2-c2ccc(-c3ccc4c(c3)Sc3cccc5cccc-4c35)cc21]

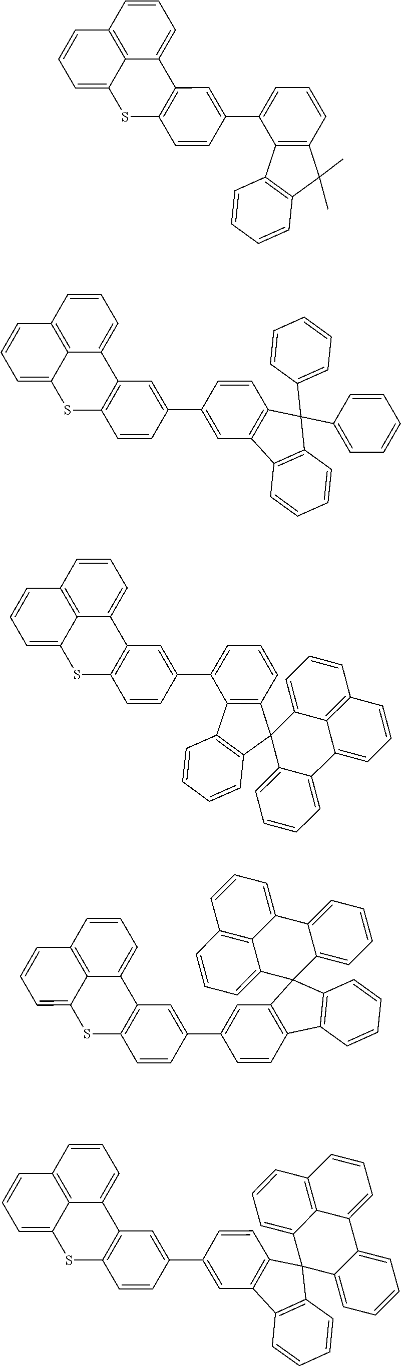 [SMILES: CC1(C)c2ccccc2-c2c(-c3ccc4c(c3)-c3cccc5cccc(c35)S4)cccc21 ; c1ccc(C2(c3ccccc3)c3ccccc3-c3cc(-c4ccc5c(c4)-c4cccc6cccc(c46)S5)ccc32)cc1 ; c1ccc2c(c1)-c1c(-c3ccc4c(c3)-c3cccc5cccc(c35)S4)cccc1C21c2ccccc2-c2cccc3cccc1c23 ; c1ccc2c(c1)-c1ccc(-c3ccc4c(c3)-c3cccc5cccc(c35)S4)cc1C21c2ccccc2-c2cccc3cccc1c23 ; c1ccc2c(c1)-c1cc(-c3ccc4c(c3)-c3cccc5cccc(c35)S4)ccc1C21c2ccccc2-c2cccc3cccc1c23]

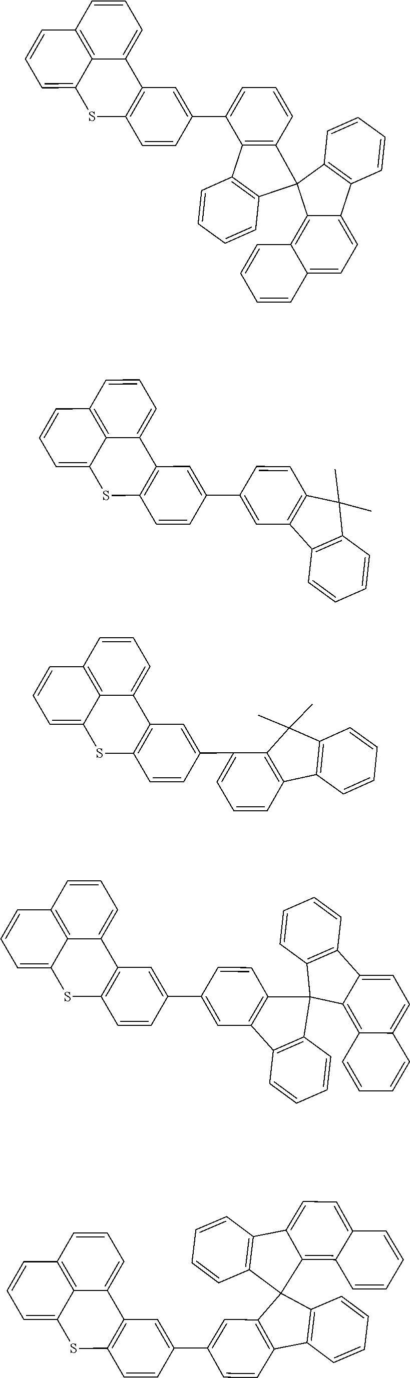 [SMILES: c1ccc2c(c1)-c1ccc3ccccc3c1C21c2ccccc2-c2c(-c3ccc4c(c3)-c3cccc5cccc(c35)S4)cccc21 ; CC1(C)c2ccccc2-c2cc(-c3ccc4c(c3)-c3cccc5cccc(c35)S4)ccc21 ; CC1(C)c2ccccc2-c2cccc(-c3ccc4c(c3)-c3cccc5cccc(c35)S4)c21 ; c1ccc2c(c1)-c1cc(-c3ccc4c(c3)-c3cccc5cccc(c35)S4)ccc1C21c2ccccc2-c2ccc3ccccc3c21 ; c1ccc2c(c1)-c1ccc(-c3ccc4c(c3)-c3cccc5cccc(c35)S4)cc1C21c2ccccc2-c2ccc3ccccc3c21]

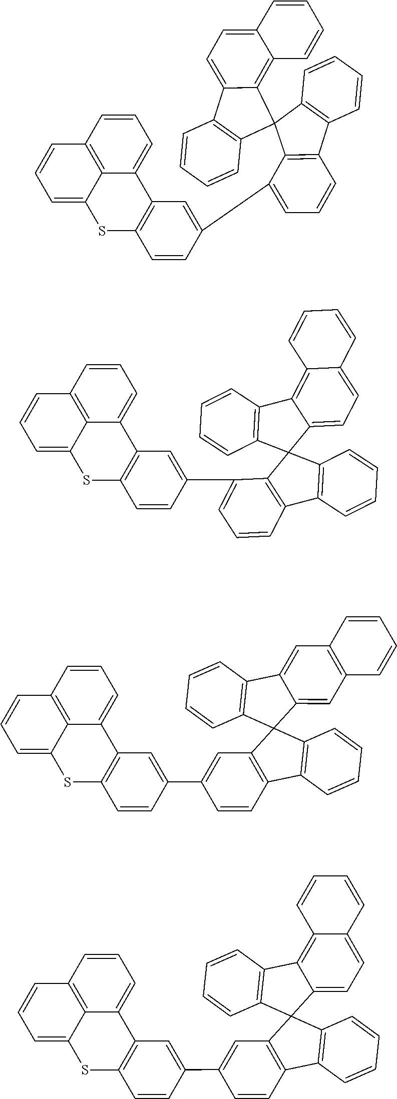 [SMILES: c1ccc2c(c1)-c1cccc(-c3ccc4c(c3)-c3cccc5cccc(c35)S4)c1C21c2ccccc2-c2ccc3ccccc3c21 ; c1ccc2c(c1)-c1cccc(-c3ccc4c(c3)-c3cccc5cccc(c35)S4)c1C21c2ccccc2-c2c1ccc1ccccc21 ; c1ccc2c(c1)-c1ccc(-c3ccc4c(c3)-c3cccc5cccc(c35)S4)cc1C21c2ccccc2-c2cc3ccccc3cc21 ; c1ccc2c(c1)-c1ccc(-c3ccc4c(c3)-c3cccc5cccc(c35)S4)cc1C21c2ccccc2-c2c1ccc1ccccc21]

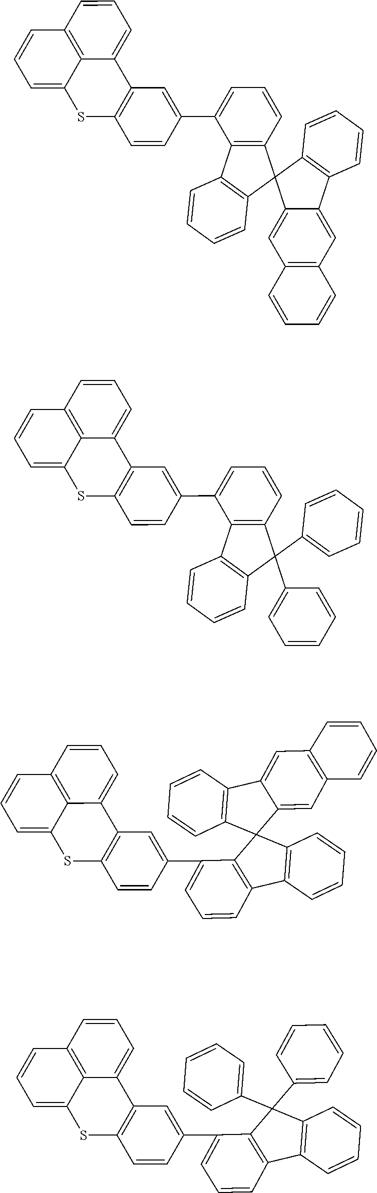 [SMILES: c1ccc2c(c1)-c1cc3ccccc3cc1C21c2ccccc2-c2c(-c3ccc4c(c3)-c3cccc5cccc(c35)S4)cccc21 ; c1ccc(C2(c3ccccc3)c3ccccc3-c3c(-c4ccc5c(c4)-c4cccc6cccc(c46)S5)cccc32)cc1 ; c1ccc2c(c1)-c1cc3ccccc3cc1C21c2ccccc2-c2cccc(-c3ccc4c(c3)-c3cccc5cccc(c35)S4)c21 ; c1ccc(C2(c3ccccc3)c3ccccc3-c3cccc(-c4ccc5c(c4)-c4cccc6cccc(c46)S5)c32)cc1]

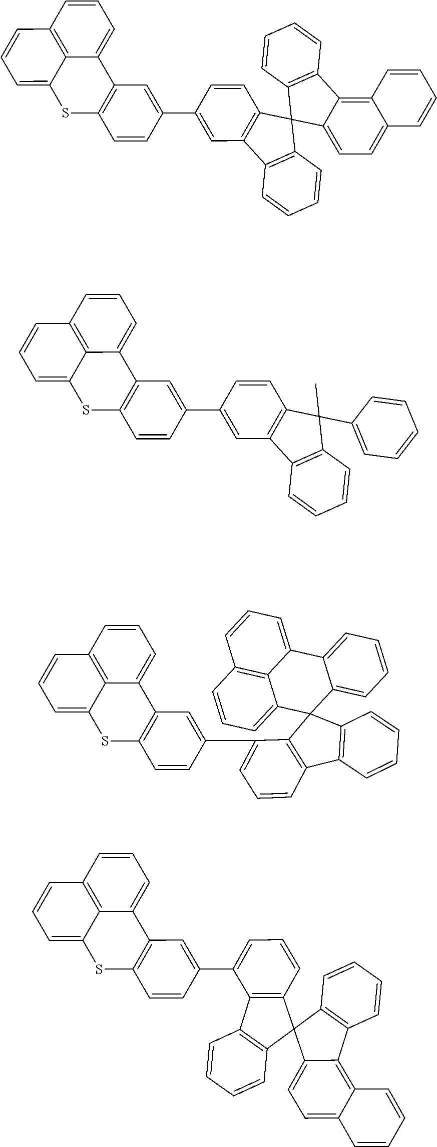 [SMILES: c1ccc2c(c1)-c1cc(-c3ccc4c(c3)-c3cccc5cccc(c35)S4)ccc1C21c2ccccc2-c2c1ccc1ccccc21 ; CC1(c2ccccc2)c2ccccc2-c2cc(-c3ccc4c(c3)-c3cccc5cccc(c35)S4)ccc21 ; c1ccc2c(c1)-c1cccc(-c3ccc4c(c3)-c3cccc5cccc(c35)S4)c1C21c2ccccc2-c2cccc3cccc1c23 ; c1ccc2c(c1)-c1c(-c3ccc4c(c3)-c3cccc5cccc(c35)S4)cccc1C21c2ccccc2-c2c1ccc1ccccc21]

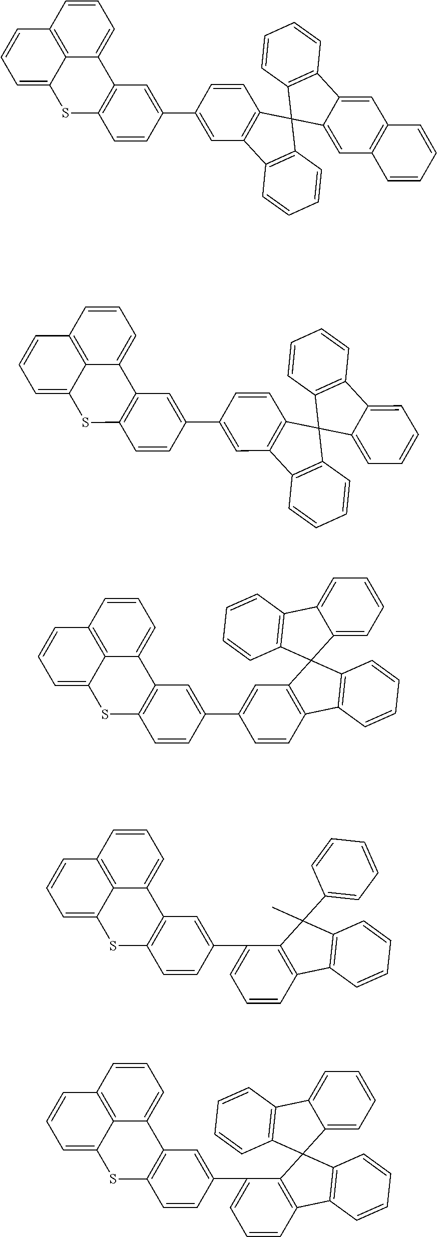 [SMILES: c1ccc2c(c1)-c1cc(-c3ccc4c(c3)-c3cccc5cccc(c35)S4)ccc1C21c2ccccc2-c2cc3ccccc3cc21 ; c1ccc2c(c1)-c1ccccc1C21c2ccccc2-c2cc(-c3ccc4c(c3)-c3cccc5cccc(c35)S4)ccc21 ; c1ccc2c(c1)-c1ccccc1C21c2ccccc2-c2ccc(-c3ccc4c(c3)-c3cccc5cccc(c35)S4)cc21 ; CC1(c2ccccc2)c2ccccc2-c2cccc(-c3ccc4c(c3)-c3cccc5cccc(c35)S4)c21 ; c1ccc2c(c1)-c1ccccc1C21c2ccccc2-c2cccc(-c3ccc4c(c3)-c3cccc5cccc(c35)S4)c21]

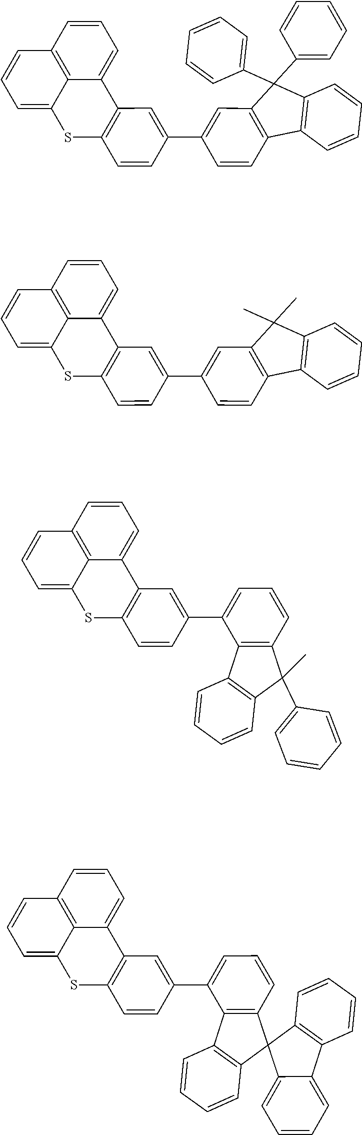 [SMILES: c1ccc(C2(c3ccccc3)c3ccccc3-c3ccc(-c4ccc5c(c4)-c4cccc6cccc(c46)S5)cc32)cc1 ; CC1(C)c2ccccc2-c2ccc(-c3ccc4c(c3)-c3cccc5cccc(c35)S4)cc21 ; CC1(c2ccccc2)c2ccccc2-c2c(-c3ccc4c(c3)-c3cccc5cccc(c35)S4)cccc21 ; c1ccc2c(c1)-c1ccccc1C21c2ccccc2-c2c(-c3ccc4c(c3)-c3cccc5cccc(c35)S4)cccc21]

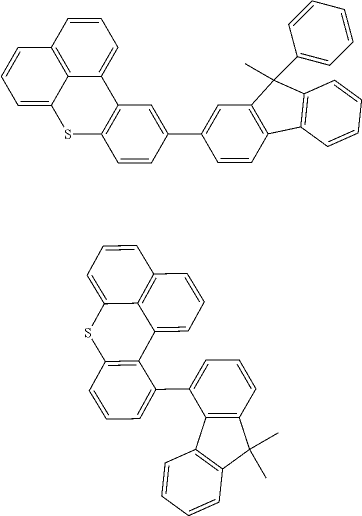 [SMILES: CC1(c2ccccc2)c2ccccc2-c2ccc(-c3ccc4c(c3)-c3cccc5cccc(c35)S4)cc21 ; CC1(C)c2ccccc2-c2c(-c3cccc4c3-c3cccc5cccc(c35)S4)cccc21]

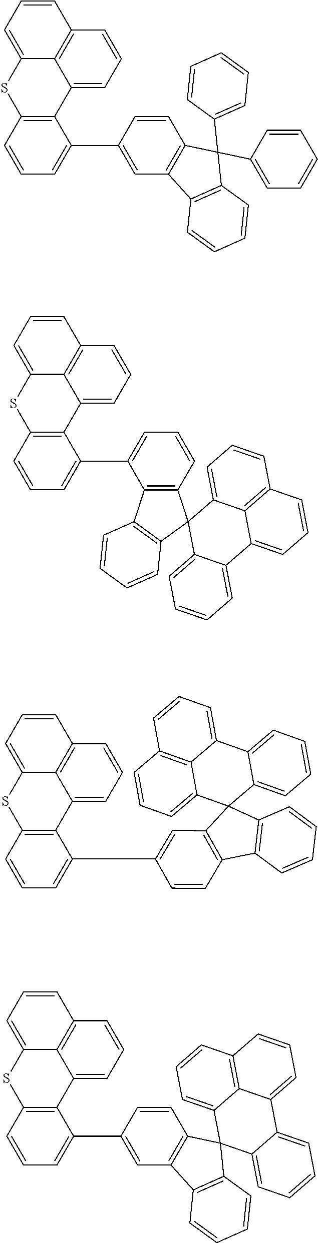 [SMILES: c1ccc(C2(c3ccccc3)c3ccccc3-c3cc(-c4cccc5c4-c4cccc6cccc(c46)S5)ccc32)cc1 ; c1ccc2c(c1)-c1c(-c3cccc4c3-c3cccc5cccc(c35)S4)cccc1C21c2ccccc2-c2cccc3cccc1c23 ; c1ccc2c(c1)-c1ccc(-c3cccc4c3-c3cccc5cccc(c35)S4)cc1C21c2ccccc2-c2cccc3cccc1c23 ; c1ccc2c(c1)-c1cc(-c3cccc4c3-c3cccc5cccc(c35)S4)ccc1C21c2ccccc2-c2cccc3cccc1c23]

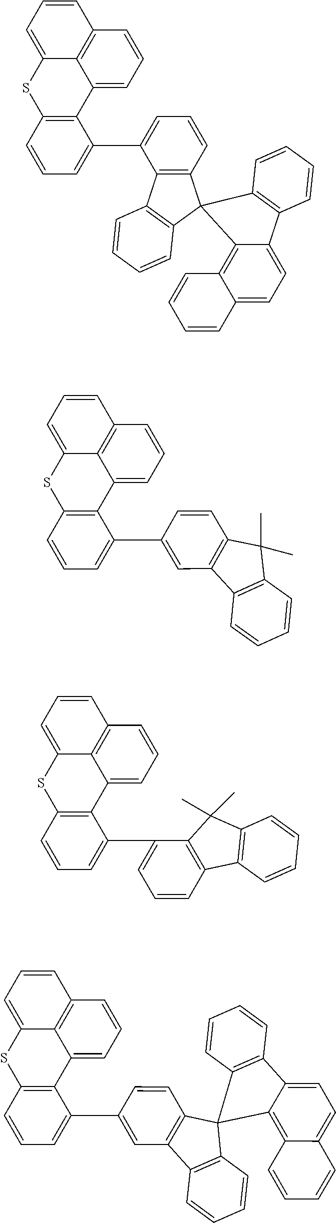 [SMILES: c1ccc2c(c1)-c1ccc3ccccc3c1C21c2ccccc2-c2c(-c3cccc4c3-c3cccc5cccc(c35)S4)cccc21 ; CC1(C)c2ccccc2-c2cc(-c3cccc4c3-c3cccc5cccc(c35)S4)ccc21 ; CC1(C)c2ccccc2-c2cccc(-c3cccc4c3-c3cccc5cccc(c35)S4)c21 ; c1ccc2c(c1)-c1cc(-c3cccc4c3-c3cccc5cccc(c35)S4)ccc1C21c2ccccc2-c2ccc3ccccc3c21]

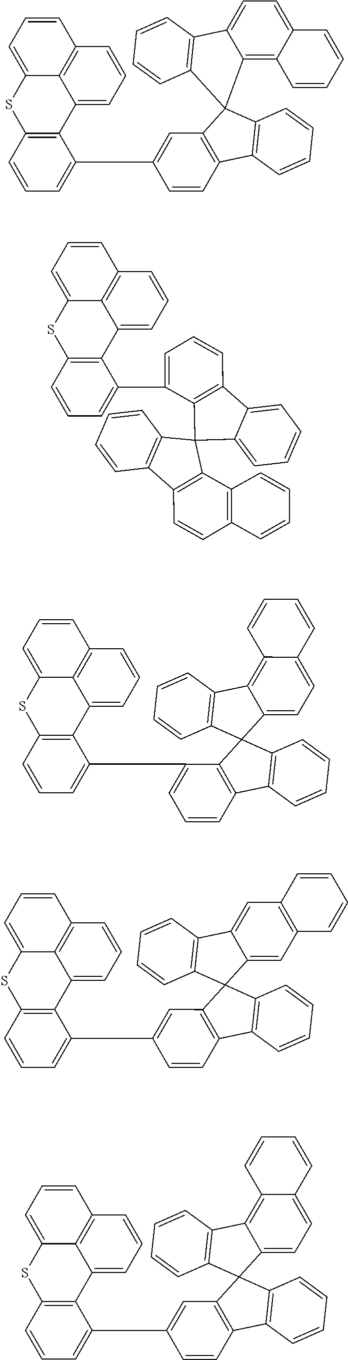 [SMILES: c1ccc2c(c1)-c1ccc(-c3cccc4c3-c3cccc5cccc(c35)S4)cc1C21c2ccccc2-c2ccc3ccccc3c21 ; c1ccc2c(c1)-c1cccc(-c3cccc4c3-c3cccc5cccc(c35)S4)c1C21c2ccccc2-c2ccc3ccccc3c21 ; c1ccc2c(c1)-c1cccc(-c3cccc4c3-c3cccc5cccc(c35)S4)c1C21c2ccccc2-c2c1ccc1ccccc21 ; c1ccc2c(c1)-c1ccc(-c3cccc4c3-c3cccc5cccc(c35)S4)cc1C21c2ccccc2-c2cc3ccccc3cc21 ; c1ccc2c(c1)-c1ccc(-c3cccc4c3-c3cccc5cccc(c35)S4)cc1C21c2ccccc2-c2c1ccc1ccccc21]

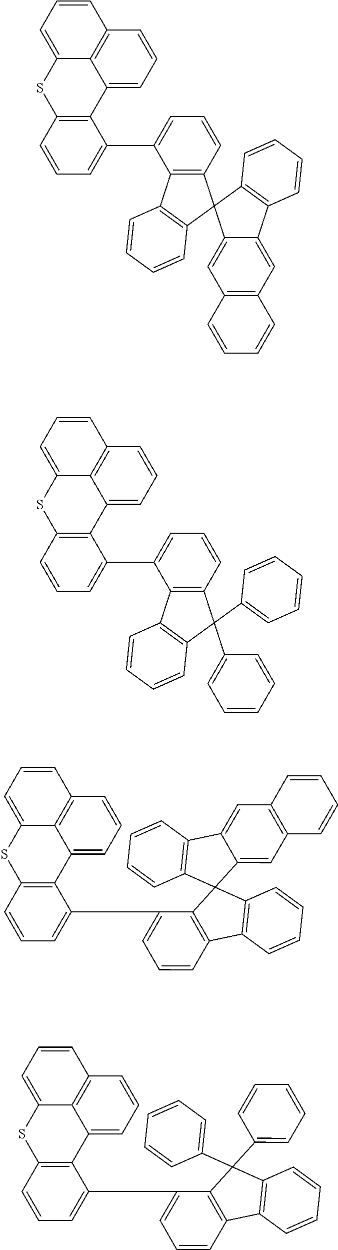 [SMILES: c1ccc2c(c1)-c1cc3ccccc3cc1C21c2ccccc2-c2c(-c3cccc4c3-c3cccc5cccc(c35)S4)cccc21 ; c1ccc(C2(c3ccccc3)c3ccccc3-c3c(-c4cccc5c4-c4cccc6cccc(c46)S5)cccc32)cc1 ; c1ccc2c(c1)-c1cc3ccccc3cc1C21c2ccccc2-c2cccc(-c3cccc4c3-c3cccc5cccc(c35)S4)c21 ; c1ccc(C2(c3ccccc3)c3ccccc3-c3cccc(-c4cccc5c4-c4cccc6cccc(c46)S5)c32)cc1]

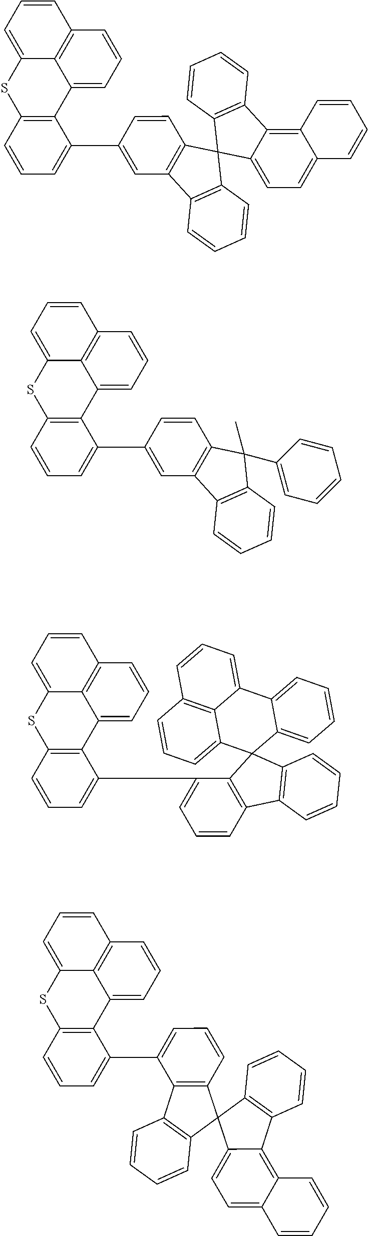 [SMILES: c1ccc2c(c1)-c1cc(-c3cccc4c3-c3cccc5cccc(c35)S4)ccc1C21c2ccccc2-c2c1ccc1ccccc21 ; CC1(c2ccccc2)c2ccccc2-c2cc(-c3cccc4c3-c3cccc5cccc(c35)S4)ccc21 ; c1ccc2c(c1)-c1cccc(-c3cccc4c3-c3cccc5cccc(c35)S4)c1C21c2ccccc2-c2cccc3cccc1c23 ; c1ccc2c(c1)-c1c(-c3cccc4c3-c3cccc5cccc(c35)S4)cccc1C21c2ccccc2-c2c1ccc1ccccc21]

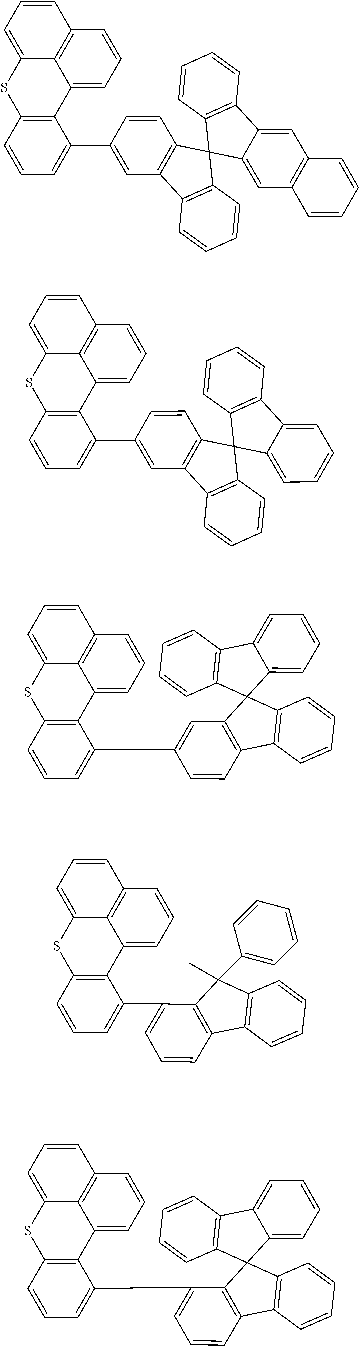 [SMILES: c1ccc2c(c1)-c1cc(-c3cccc4c3-c3cccc5cccc(c35)S4)ccc1C21c2ccccc2-c2cc3ccccc3cc21 ; c1ccc2c(c1)-c1ccccc1C21c2ccccc2-c2cc(-c3cccc4c3-c3cccc5cccc(c35)S4)ccc21 ; c1ccc2c(c1)-c1ccccc1C21c2ccccc2-c2ccc(-c3cccc4c3-c3cccc5cccc(c35)S4)cc21 ; CC1(c2ccccc2)c2ccccc2-c2cccc(-c3cccc4c3-c3cccc5cccc(c35)S4)c21 ; c1ccc2c(c1)-c1ccccc1C21c2ccccc2-c2cccc(-c3cccc4c3-c3cccc5cccc(c35)S4)c21]

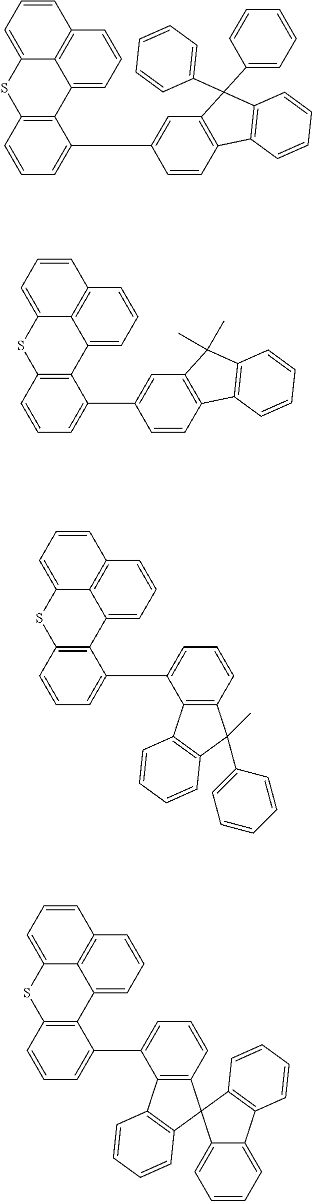 [SMILES: c1ccc(C2(c3ccccc3)c3ccccc3-c3ccc(-c4cccc5c4-c4cccc6cccc(c46)S5)cc32)cc1 ; CC1(C)c2ccccc2-c2ccc(-c3cccc4c3-c3cccc5cccc(c35)S4)cc21 ; CC1(c2ccccc2)c2ccccc2-c2c(-c3cccc4c3-c3cccc5cccc(c35)S4)cccc21 ; c1ccc2c(c1)-c1ccccc1C21c2ccccc2-c2c(-c3cccc4c3-c3cccc5cccc(c35)S4)cccc21]

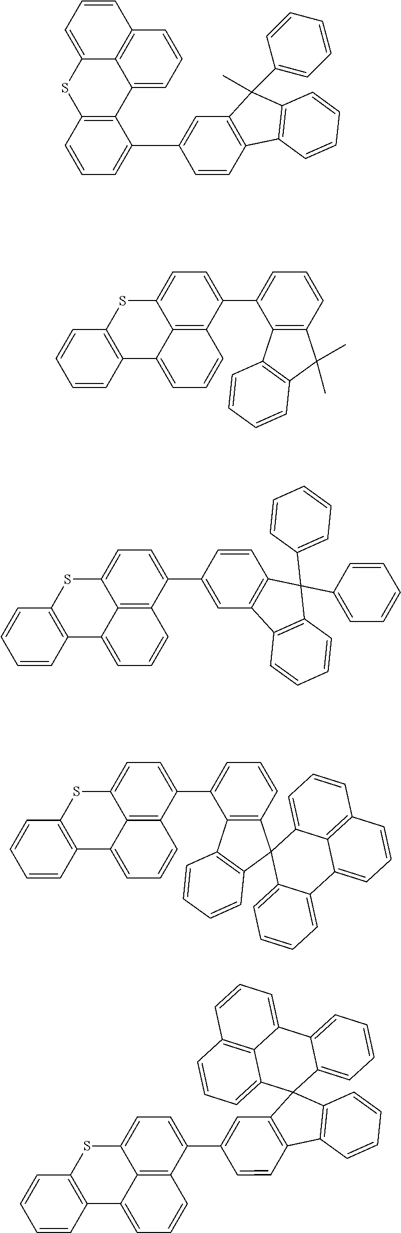 [SMILES: CC1(c2ccccc2)c2ccccc2-c2ccc(-c3cccc4c3-c3cccc5cccc(c35)S4)cc21 ; CC1(C)c2ccccc2-c2c(-c3ccc4c5c(cccc35)-c3ccccc3S4)cccc21 ; c1ccc(C2(c3ccccc3)c3ccccc3-c3cc(-c4ccc5c6c(cccc46)-c4ccccc4S5)ccc32)cc1 ; c1ccc2c(c1)Sc1ccc(-c3cccc4c3-c3ccccc3C43c4ccccc4-c4cccc5cccc3c45)c3cccc-2c13 ; c1ccc2c(c1)Sc1ccc(-c3ccc4c(c3)C3(c5ccccc5-4)c4ccccc4-c4cccc5cccc3c45)c3cccc-2c13]

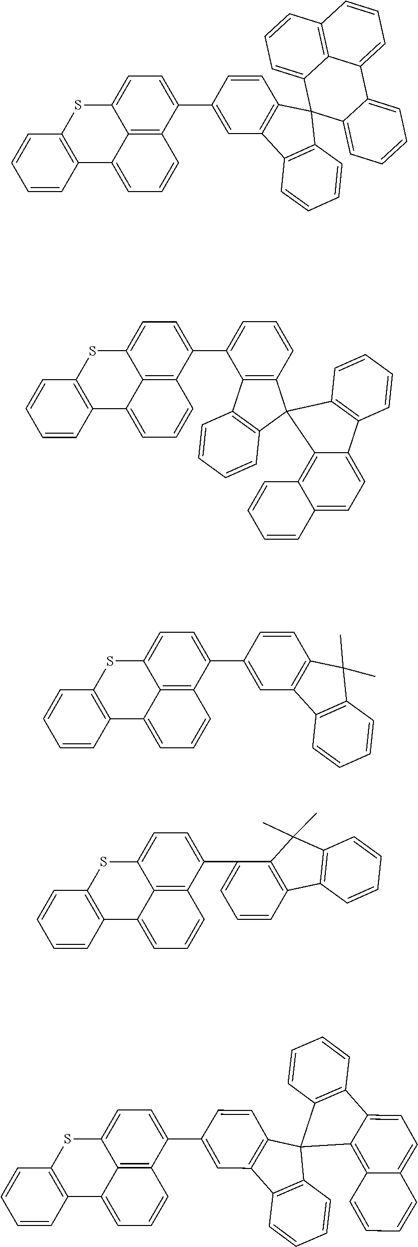 [SMILES: c1ccc2c(c1)Sc1ccc(-c3ccc4c(c3)-c3ccccc3C43c4ccccc4-c4cccc5cccc3c45)c3cccc-2c13 ; c1ccc2c(c1)Sc1ccc(-c3cccc4c3-c3ccccc3C43c4ccccc4-c4ccc5ccccc5c43)c3cccc-2c13 ; CC1(C)c2ccccc2-c2cc(-c3ccc4c5c(cccc35)-c3ccccc3S4)ccc21 ; CC1(C)c2ccccc2-c2cccc(-c3ccc4c5c(cccc35)-c3ccccc3S4)c21 ; c1ccc2c(c1)Sc1ccc(-c3ccc4c(c3)-c3ccccc3C43c4ccccc4-c4ccc5ccccc5c43)c3cccc-2c13]

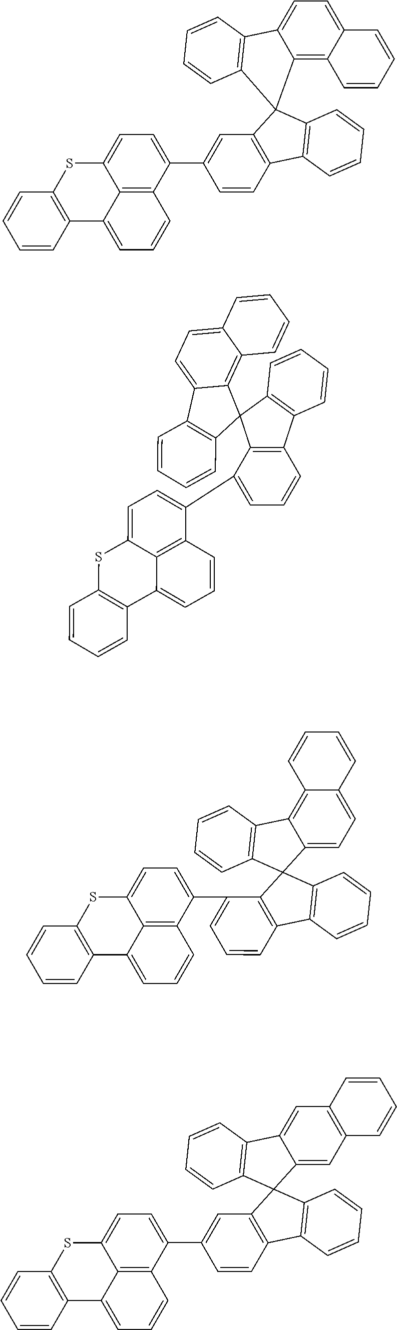 [SMILES: c1ccc2c(c1)Sc1ccc(-c3ccc4c(c3)C3(c5ccccc5-4)c4ccccc4-c4ccc5ccccc5c43)c3cccc-2c13 ; c1ccc2c(c1)Sc1ccc(-c3cccc4c3C3(c5ccccc5-4)c4ccccc4-c4ccc5ccccc5c43)c3cccc-2c13 ; c1ccc2c(c1)Sc1ccc(-c3cccc4c3C3(c5ccccc5-4)c4ccccc4-c4c3ccc3ccccc43)c3cccc-2c13 ; c1ccc2c(c1)Sc1ccc(-c3ccc4c(c3)C3(c5ccccc5-4)c4ccccc4-c4cc5ccccc5cc43)c3cccc-2c13]

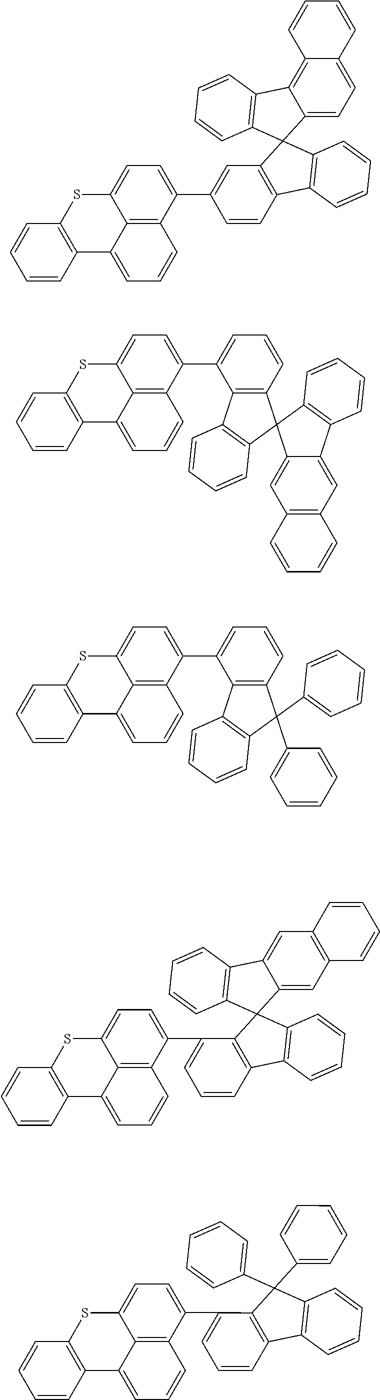 [SMILES: c1ccc2c(c1)Sc1ccc(-c3ccc4c(c3)C3(c5ccccc5-4)c4ccccc4-c4c3ccc3ccccc43)c3cccc-2c13 ; c1ccc2c(c1)Sc1ccc(-c3cccc4c3-c3ccccc3C43c4ccccc4-c4cc5ccccc5cc43)c3cccc-2c13 ; c1ccc(C2(c3ccccc3)c3ccccc3-c3c(-c4ccc5c6c(cccc46)-c4ccccc4S5)cccc32)cc1 ; c1ccc2c(c1)Sc1ccc(-c3cccc4c3C3(c5ccccc5-c5cc6ccccc6cc53)c3ccccc3-4)c3cccc-2c13 ; c1ccc(C2(c3ccccc3)c3ccccc3-c3cccc(-c4ccc5c6c(cccc46)-c4ccccc4S5)c32)cc1]

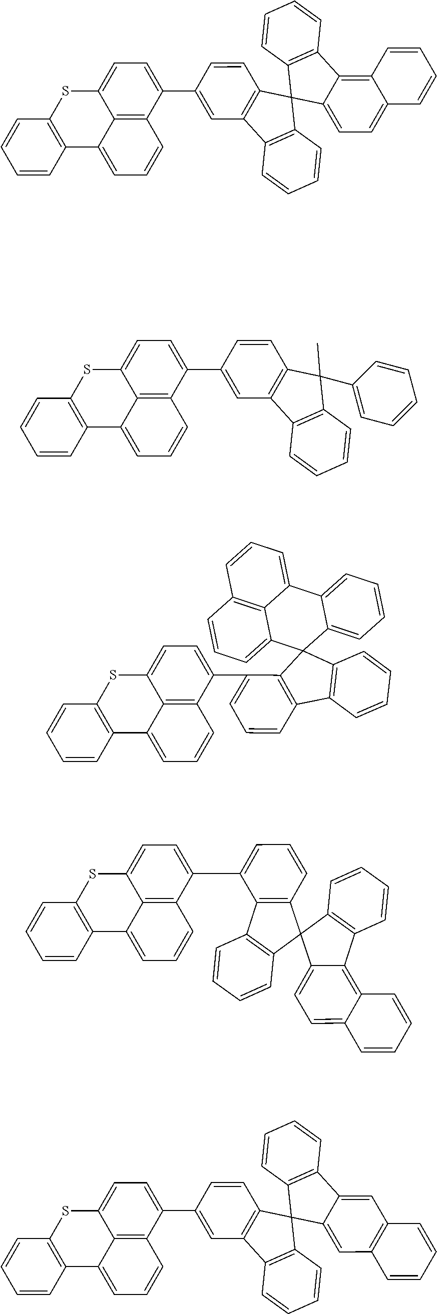 [SMILES: c1ccc2c(c1)Sc1ccc(-c3ccc4c(c3)-c3ccccc3C43c4ccccc4-c4c3ccc3ccccc43)c3cccc-2c13 ; CC1(c2ccccc2)c2ccccc2-c2cc(-c3ccc4c5c(cccc35)-c3ccccc3S4)ccc21 ; c1ccc2c(c1)Sc1ccc(-c3cccc4c3C3(c5ccccc5-4)c4ccccc4-c4cccc5cccc3c45)c3cccc-2c13 ; c1ccc2c(c1)Sc1ccc(-c3cccc4c3-c3ccccc3C43c4ccccc4-c4c3ccc3ccccc43)c3cccc-2c13 ; c1ccc2c(c1)Sc1ccc(-c3ccc4c(c3)-c3ccccc3C43c4ccccc4-c4cc5ccccc5cc43)c3cccc-2c13]

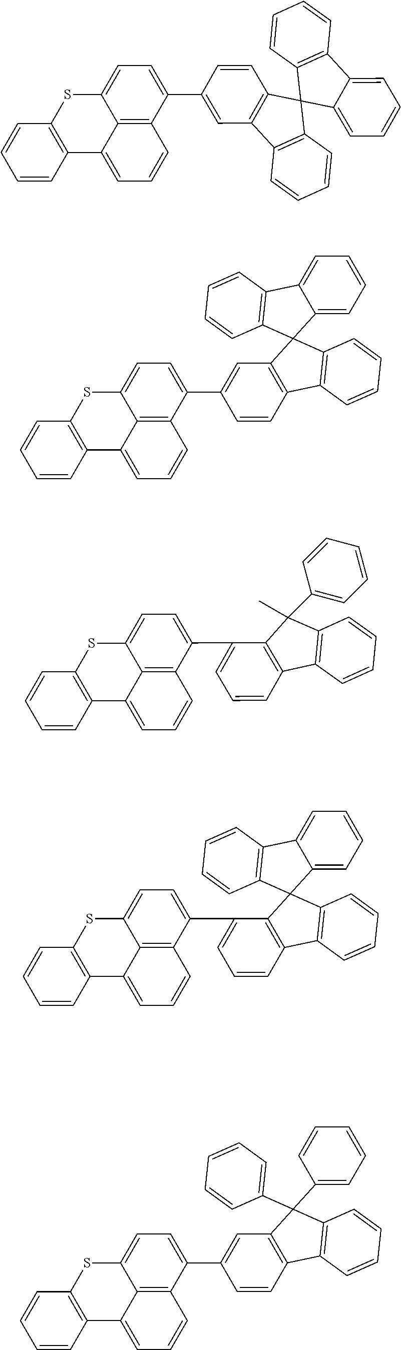 [SMILES: c1ccc2c(c1)Sc1ccc(-c3ccc4c(c3)-c3ccccc3C43c4ccccc4-c4ccccc43)c3cccc-2c13 ; c1ccc2c(c1)Sc1ccc(-c3ccc4c(c3)C3(c5ccccc5-c5ccccc53)c3ccccc3-4)c3cccc-2c13 ; CC1(c2ccccc2)c2ccccc2-c2cccc(-c3ccc4c5c(cccc35)-c3ccccc3S4)c21 ; c1ccc2c(c1)Sc1ccc(-c3cccc4c3C3(c5ccccc5-c5ccccc53)c3ccccc3-4)c3cccc-2c13 ; c1ccc(C2(c3ccccc3)c3ccccc3-c3ccc(-c4ccc5c6c(cccc46)-c4ccccc4S5)cc32)cc1]

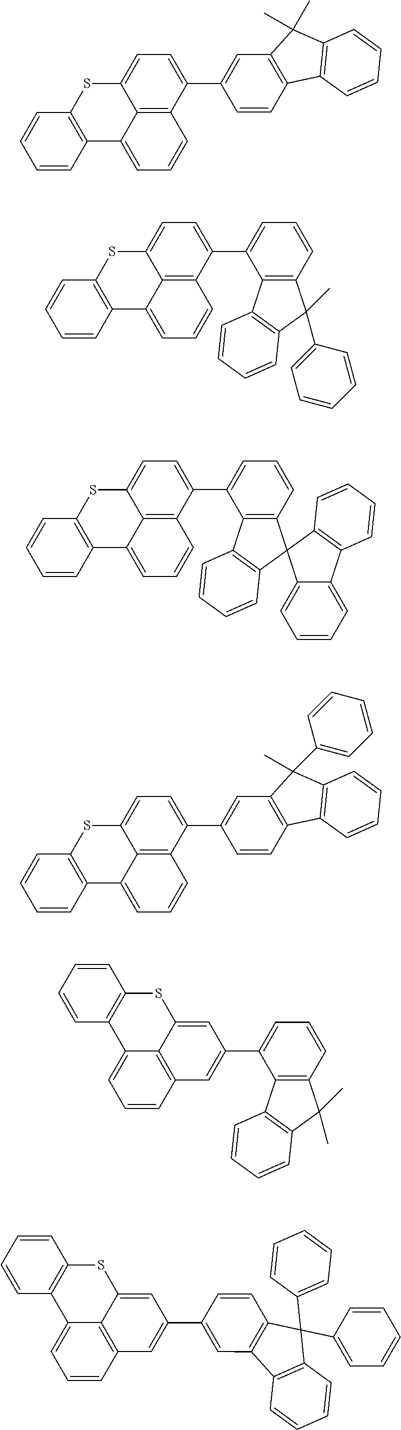 [SMILES: CC1(C)c2ccccc2-c2ccc(-c3ccc4c5c(cccc35)-c3ccccc3S4)cc21 ; CC1(c2ccccc2)c2ccccc2-c2c(-c3ccc4c5c(cccc35)-c3ccccc3S4)cccc21 ; c1ccc2c(c1)Sc1ccc(-c3cccc4c3-c3ccccc3C43c4ccccc4-c4ccccc43)c3cccc-2c13 ; CC1(c2ccccc2)c2ccccc2-c2ccc(-c3ccc4c5c(cccc35)-c3ccccc3S4)cc21 ; CC1(C)c2ccccc2-c2c(-c3cc4c5c(cccc5c3)-c3ccccc3S4)cccc21 ; c1ccc(C2(c3ccccc3)c3ccccc3-c3cc(-c4cc5c6c(cccc6c4)-c4ccccc4S5)ccc32)cc1]

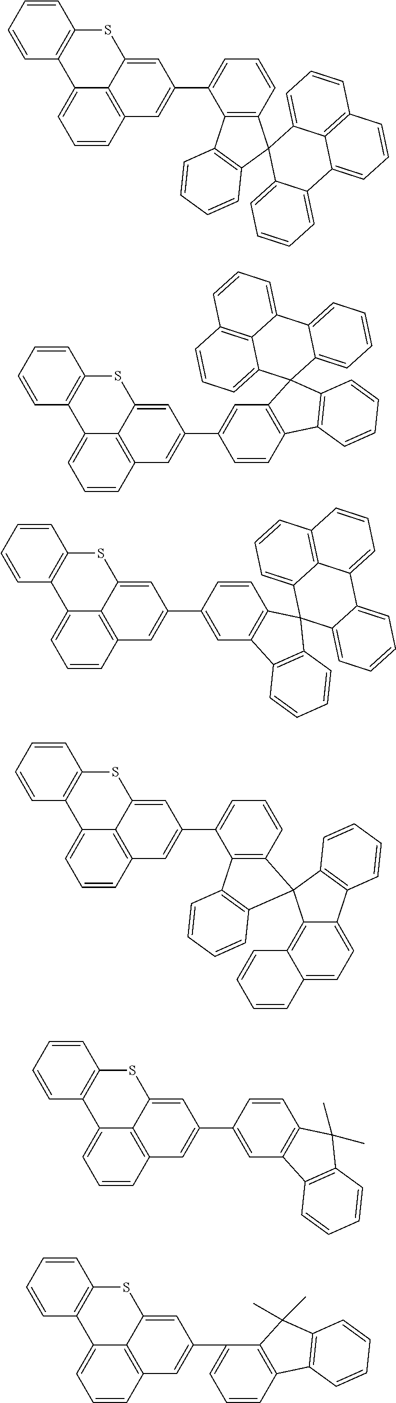

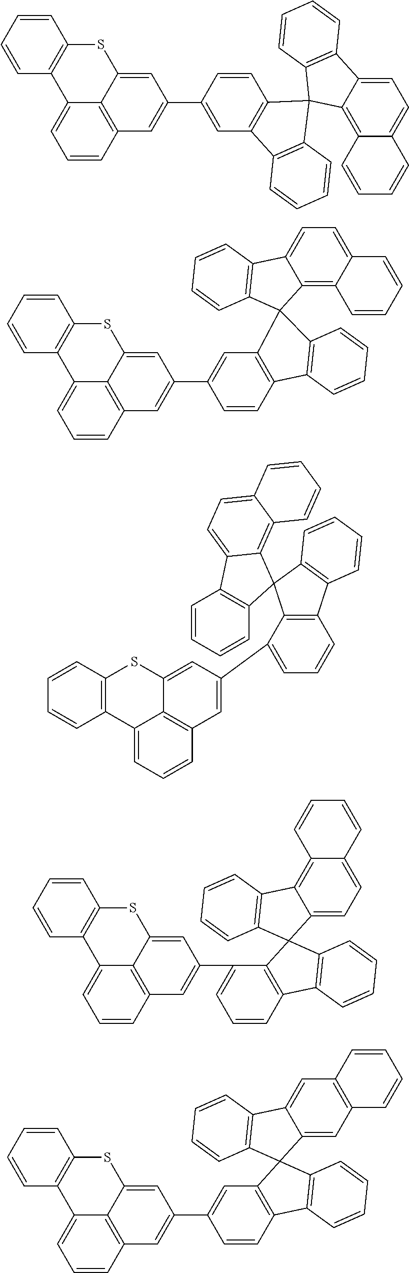

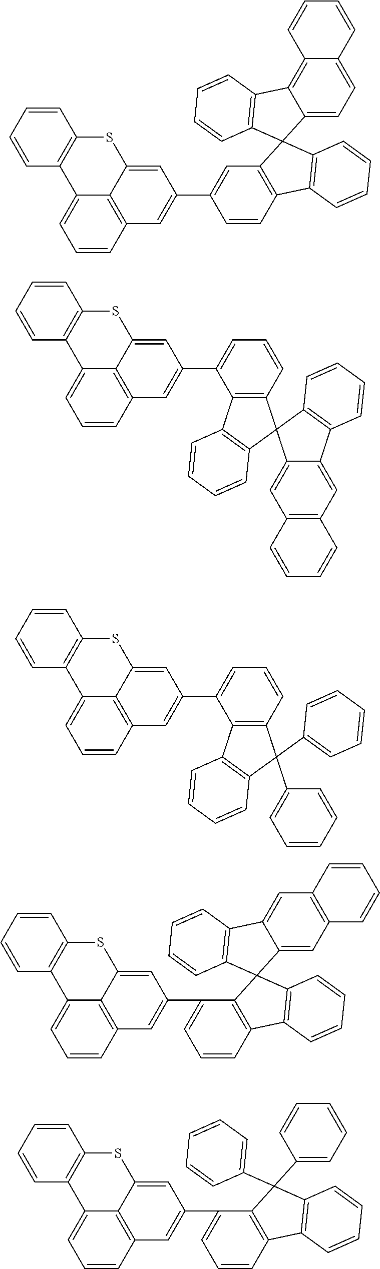

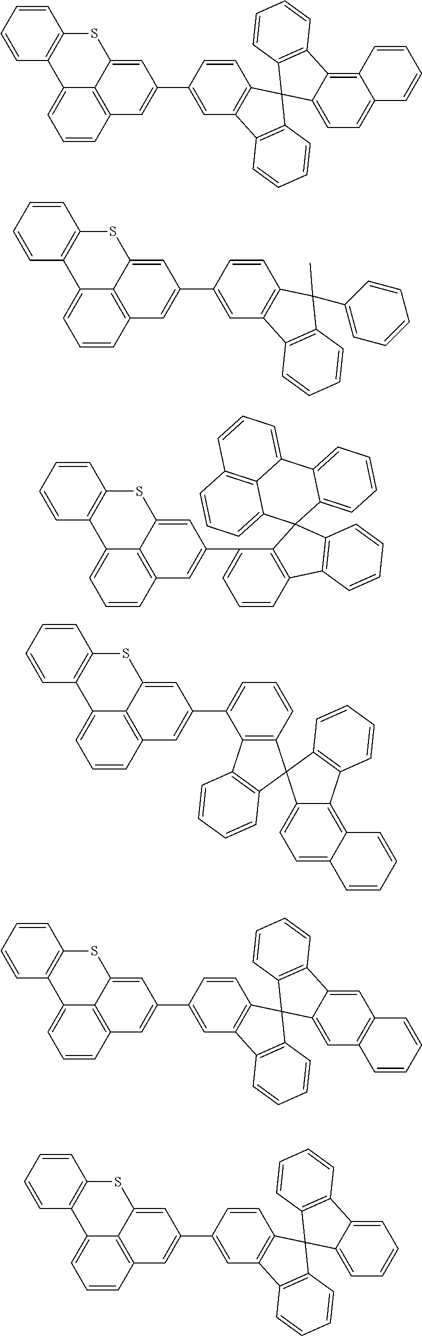

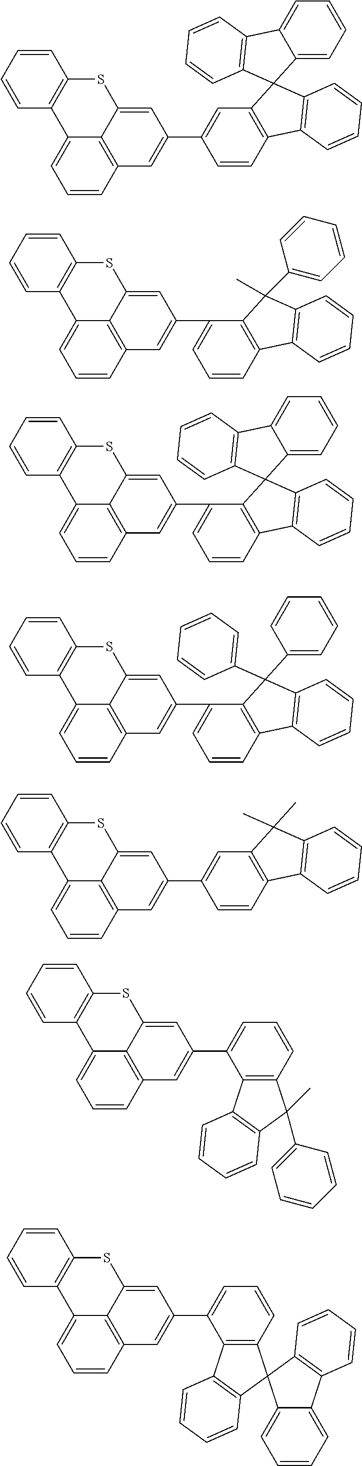

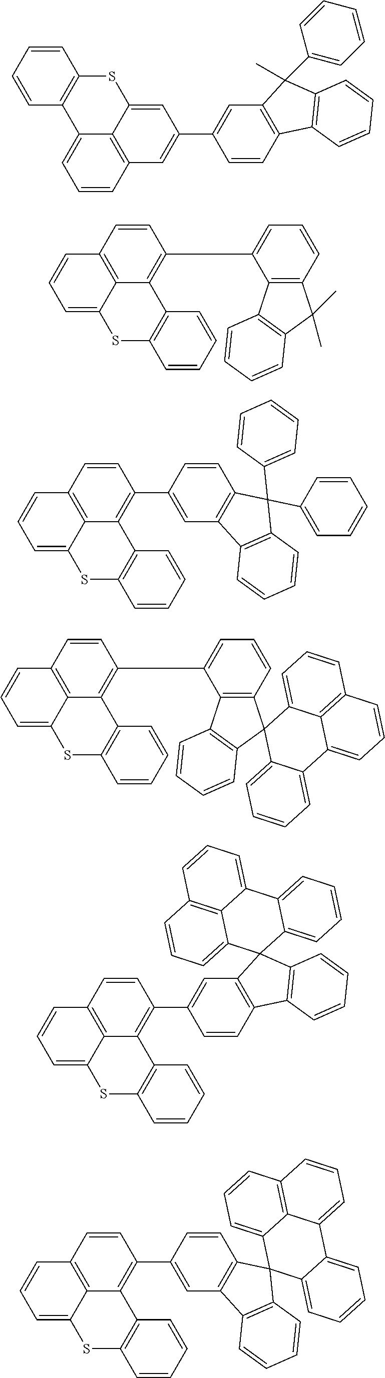

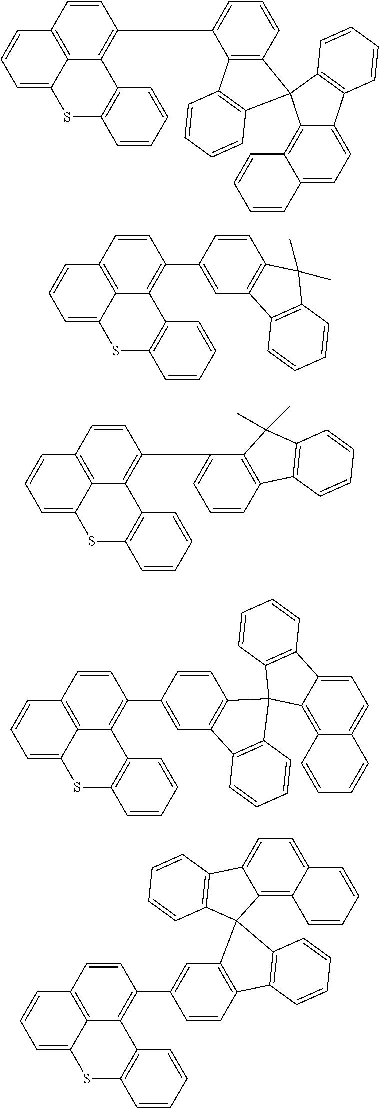

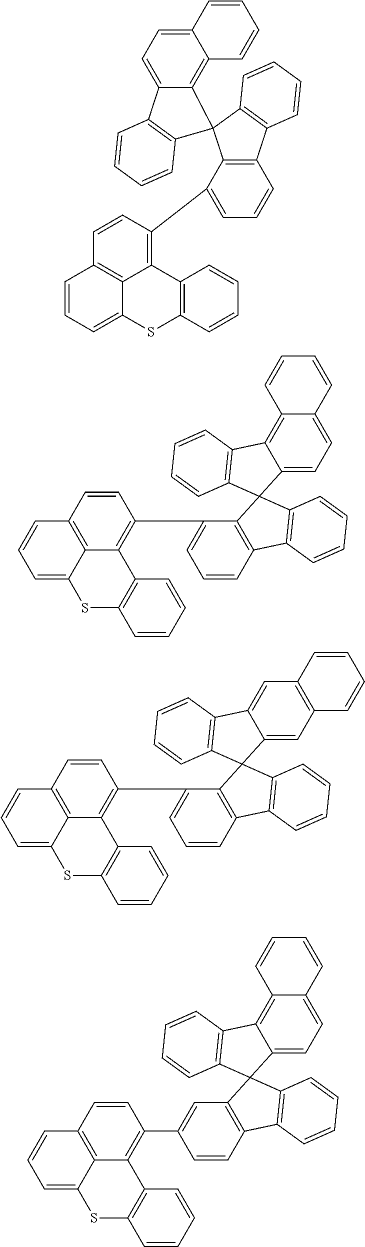 [SMILES: c1ccc2c(c1)Sc1cccc3ccc(-c4cccc5c4C4(c6ccccc6-5)c5ccccc5-c5ccc6ccccc6c54)c-2c13 ; c1ccc2c(c1)Sc1cccc3ccc(-c4cccc5c4C4(c6ccccc6-5)c5ccccc5-c5c4ccc4ccccc54)c-2c13 ; c1ccc2c(c1)Sc1cccc3ccc(-c4cccc5c4C4(c6ccccc6-c6cc7ccccc7cc64)c4ccccc4-5)c-2c13 ; c1ccc2c(c1)Sc1cccc3ccc(-c4ccc5c(c4)C4(c6ccccc6-5)c5ccccc5-c5c4ccc4ccccc54)c-2c13]

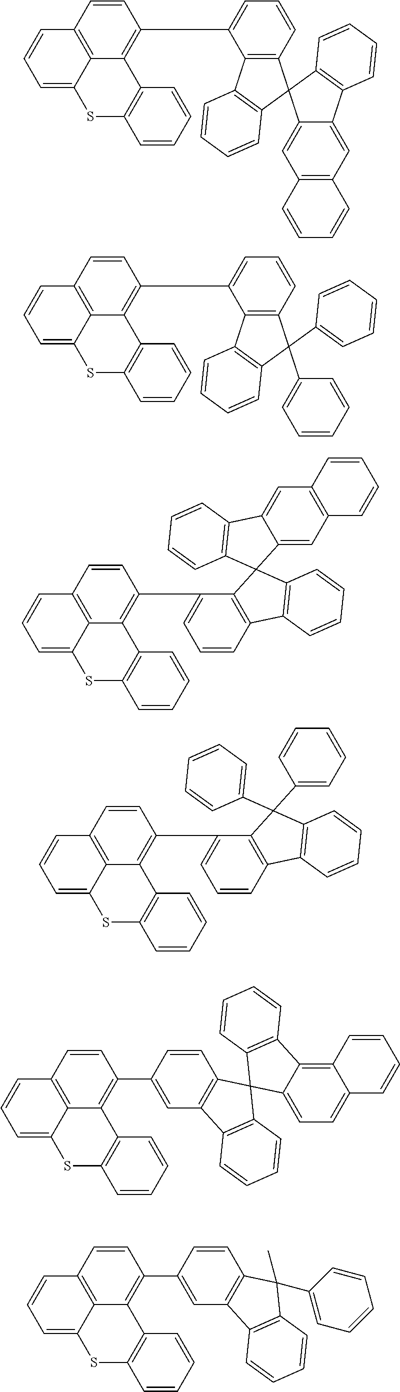

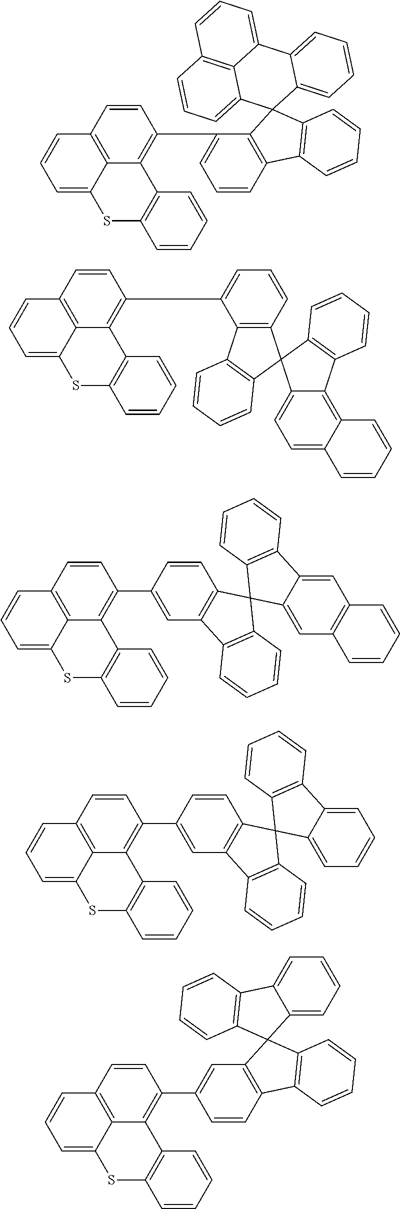

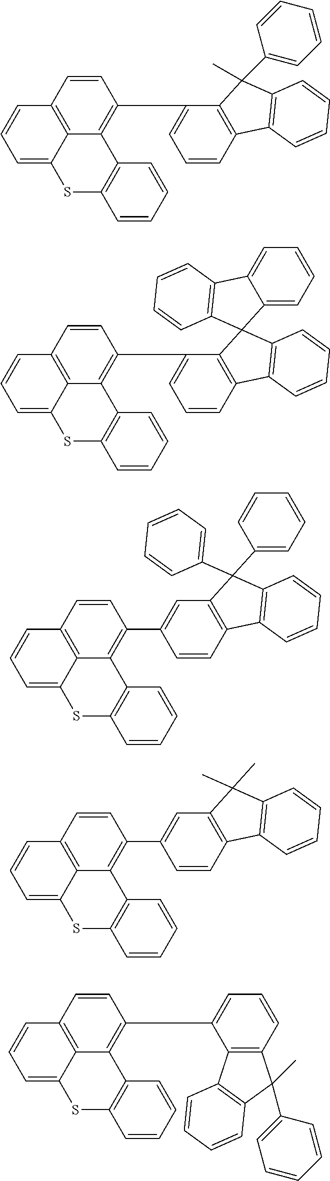

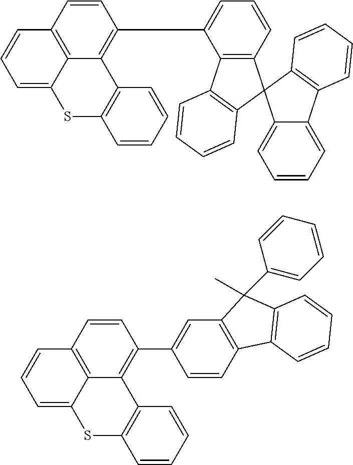

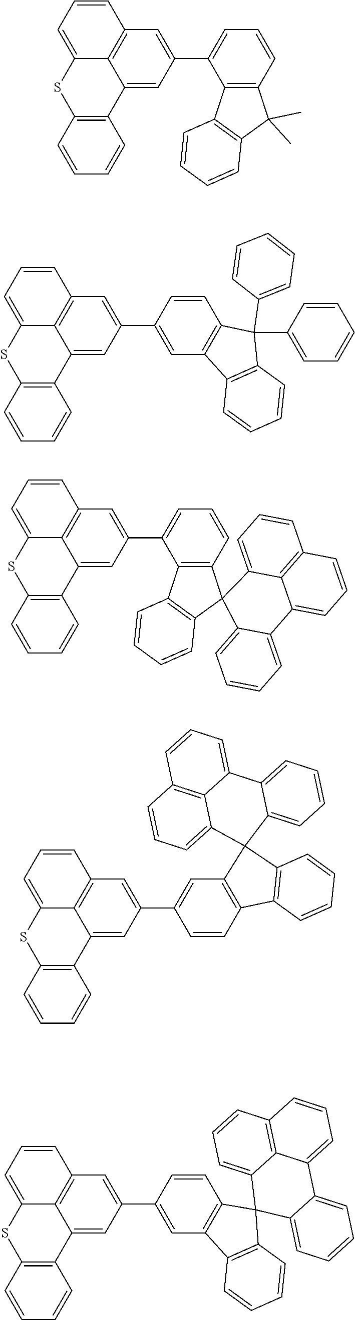

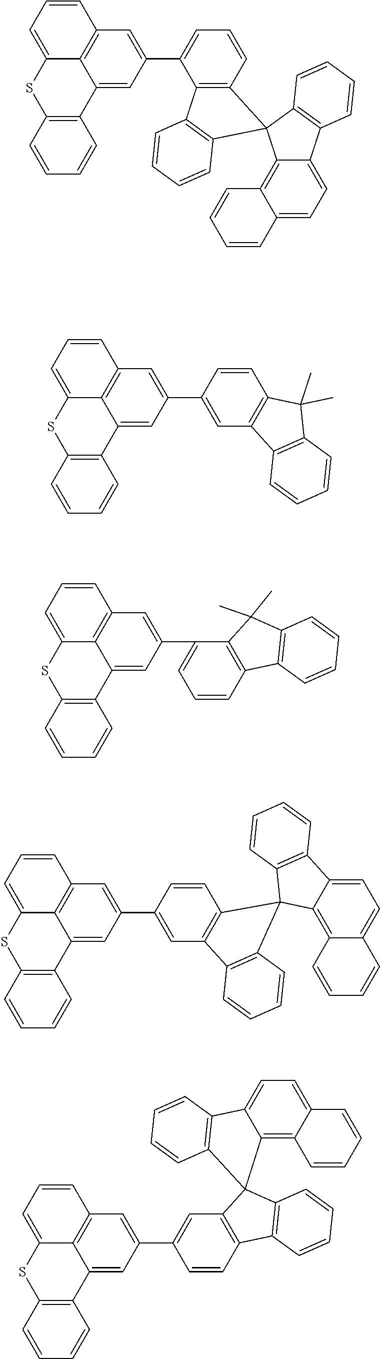 [SMILES: c1ccc2c(c1)Sc1cccc3cc(-c4cccc5c4-c4ccccc4C54c5ccccc5-c5ccc6ccccc6c54)cc-2c13 ; CC1(C)c2ccccc2-c2cc(-c3cc4c5c(cccc5c3)Sc3ccccc3-4)ccc21 ; CC1(C)c2ccccc2-c2cccc(-c3cc4c5c(cccc5c3)Sc3ccccc3-4)c21 ; c1ccc2c(c1)Sc1cccc3cc(-c4ccc5c(c4)-c4ccccc4C54c5ccccc5-c5ccc6ccccc6c54)cc-2c13 ; c1ccc2c(c1)Sc1cccc3cc(-c4ccc5c(c4)C4(c6ccccc6-5)c5ccccc5-c5ccc6ccccc6c54)cc-2c13]

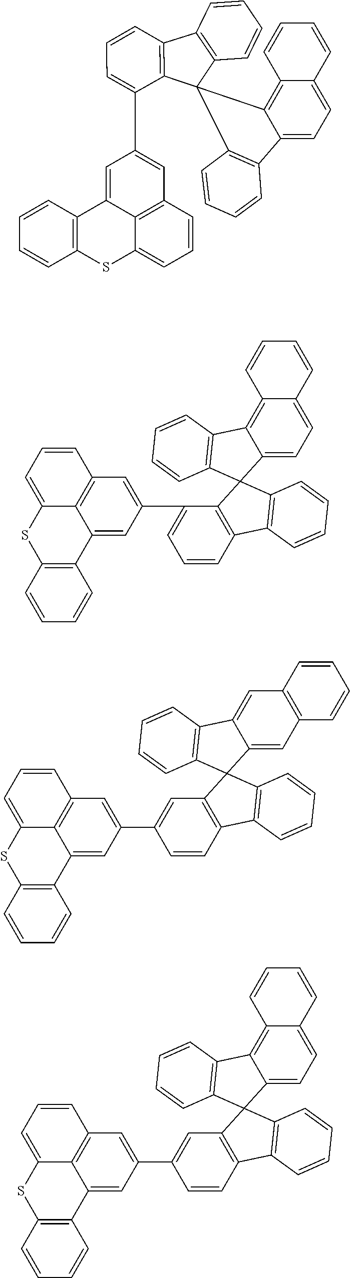 [SMILES: c1ccc2c(c1)Sc1cccc3cc(-c4cccc5c4C4(c6ccccc6-5)c5ccccc5-c5ccc6ccccc6c54)cc-2c13 ; c1ccc2c(c1)Sc1cccc3cc(-c4cccc5c4C4(c6ccccc6-5)c5ccccc5-c5c4ccc4ccccc54)cc-2c13 ; c1ccc2c(c1)Sc1cccc3cc(-c4ccc5c(c4)C4(c6ccccc6-5)c5ccccc5-c5cc6ccccc6cc54)cc-2c13 ; c1ccc2c(c1)Sc1cccc3cc(-c4ccc5c(c4)C4(c6ccccc6-5)c5ccccc5-c5c4ccc4ccccc54)cc-2c13]

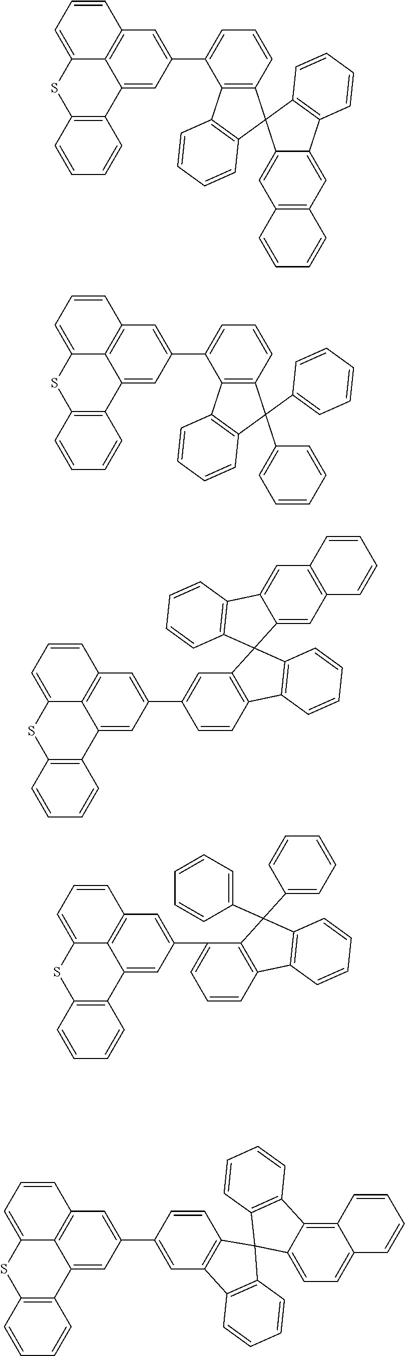

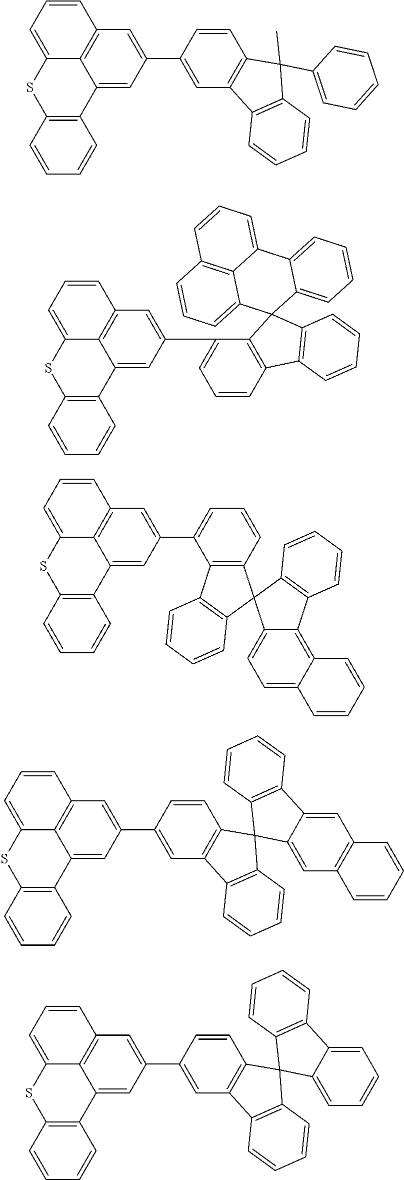

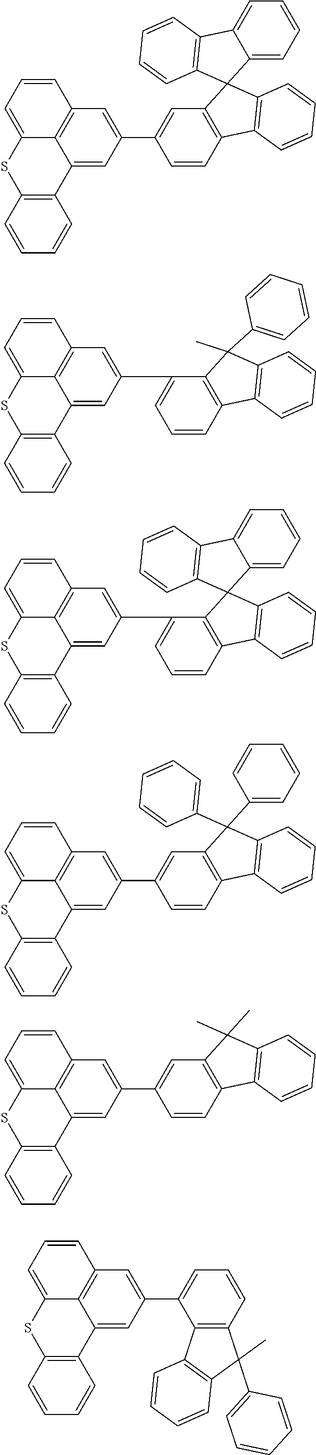

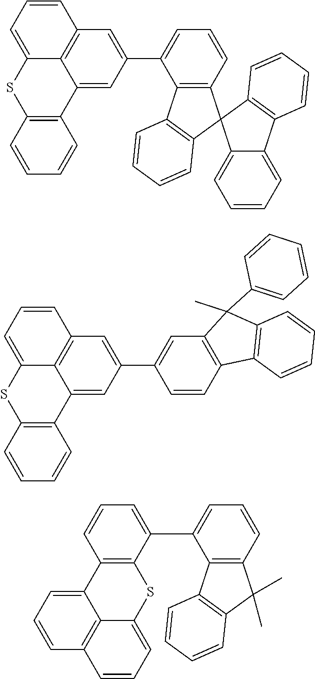

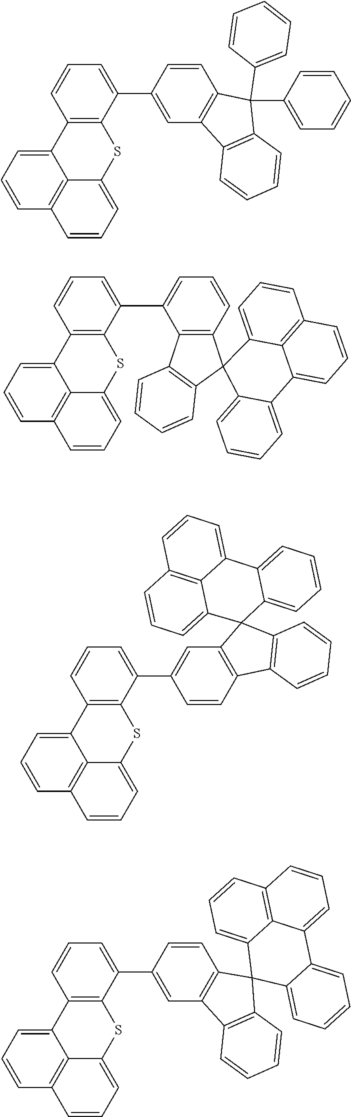

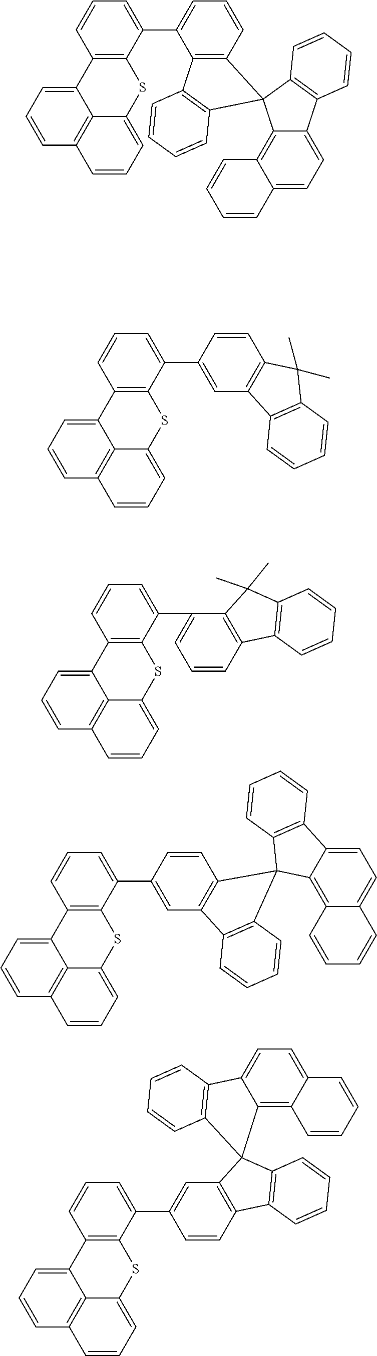 [SMILES: c1ccc2c(c1)-c1ccc3ccccc3c1C21c2ccccc2-c2c(-c3cccc4c3Sc3cccc5cccc-4c35)cccc21 ; CC1(C)c2ccccc2-c2cc(-c3cccc4c3Sc3cccc5cccc-4c35)ccc21 ; CC1(C)c2ccccc2-c2cccc(-c3cccc4c3Sc3cccc5cccc-4c35)c21 ; c1ccc2c(c1)-c1cc(-c3cccc4c3Sc3cccc5cccc-4c35)ccc1C21c2ccccc2-c2ccc3ccccc3c21 ; c1ccc2c(c1)-c1ccc(-c3cccc4c3Sc3cccc5cccc-4c35)cc1C21c2ccccc2-c2ccc3ccccc3c21]

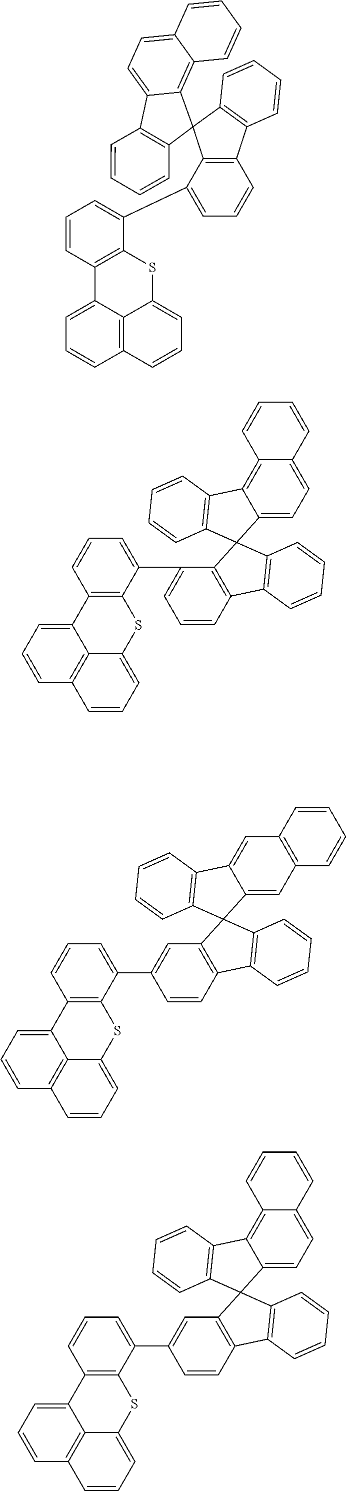

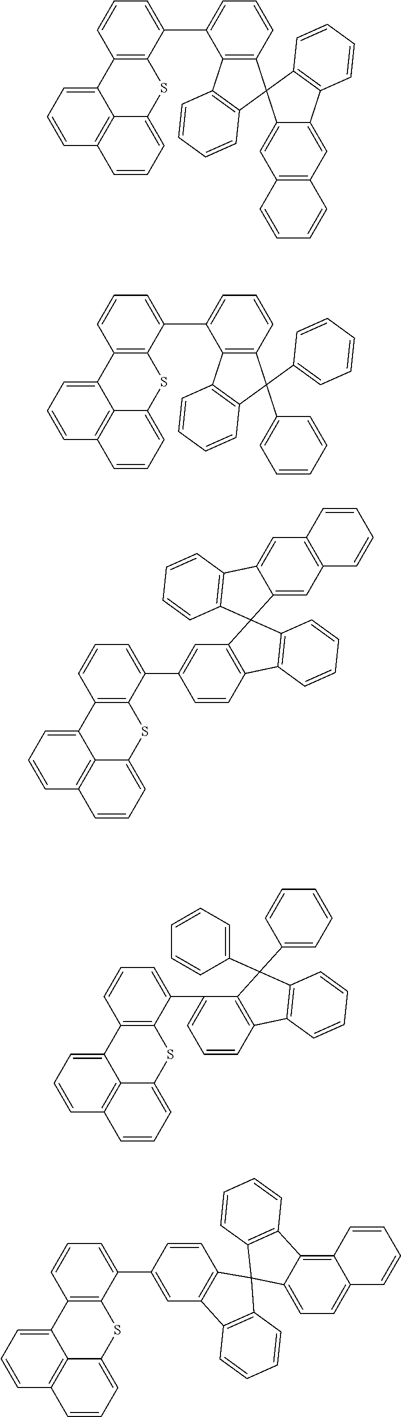

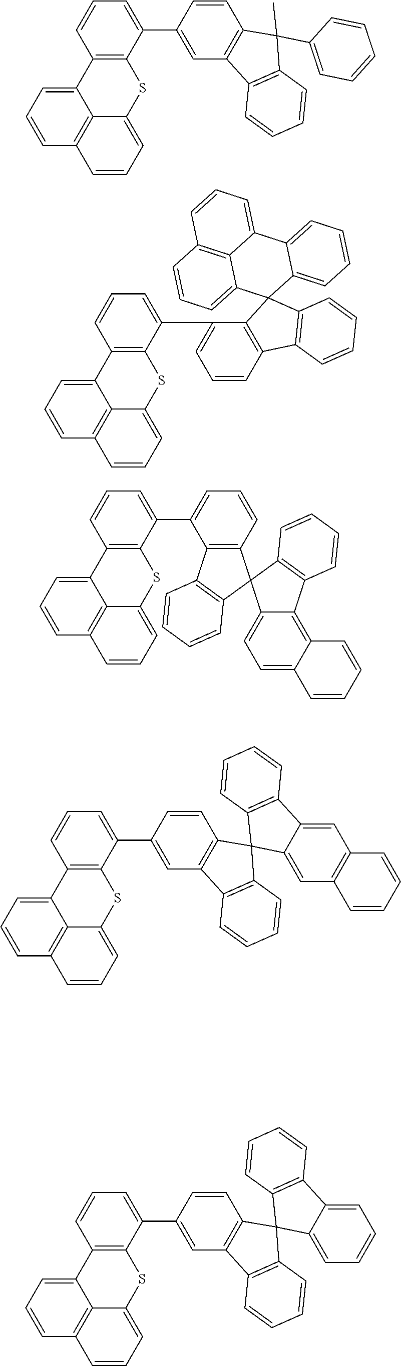

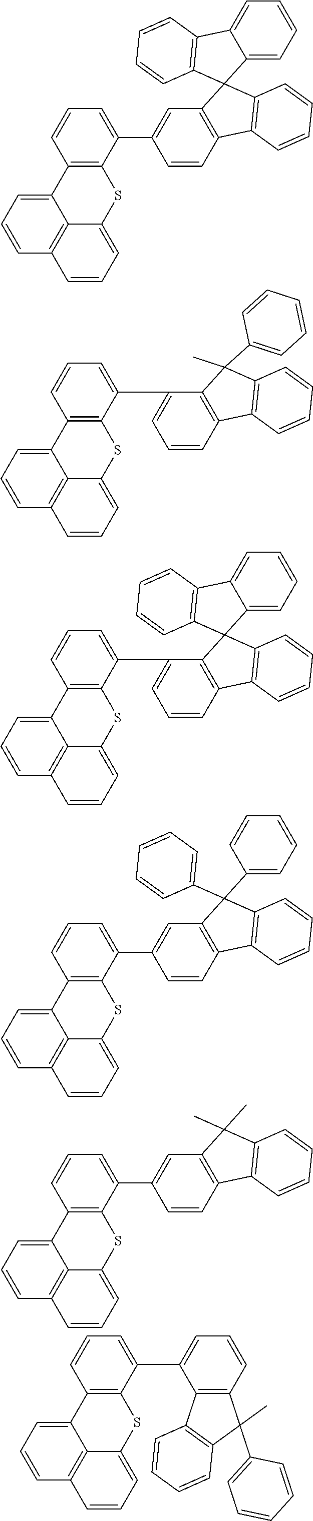

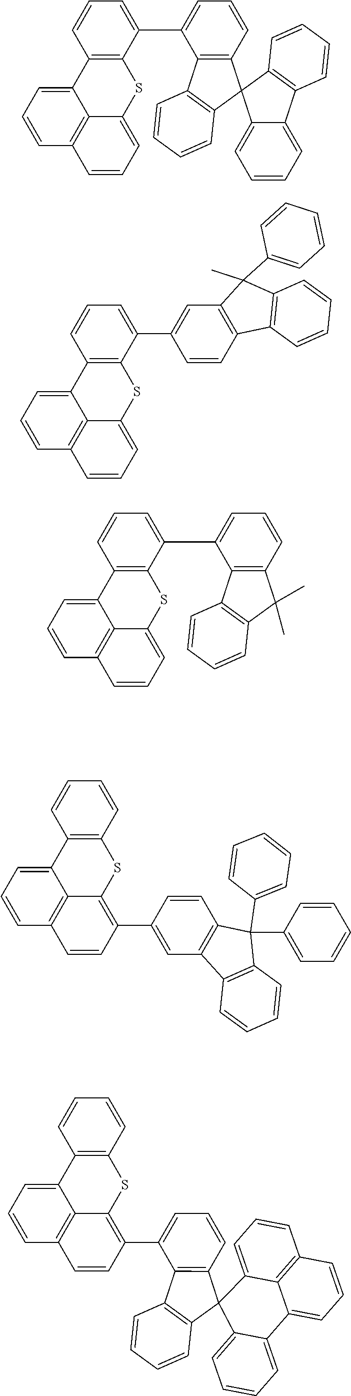

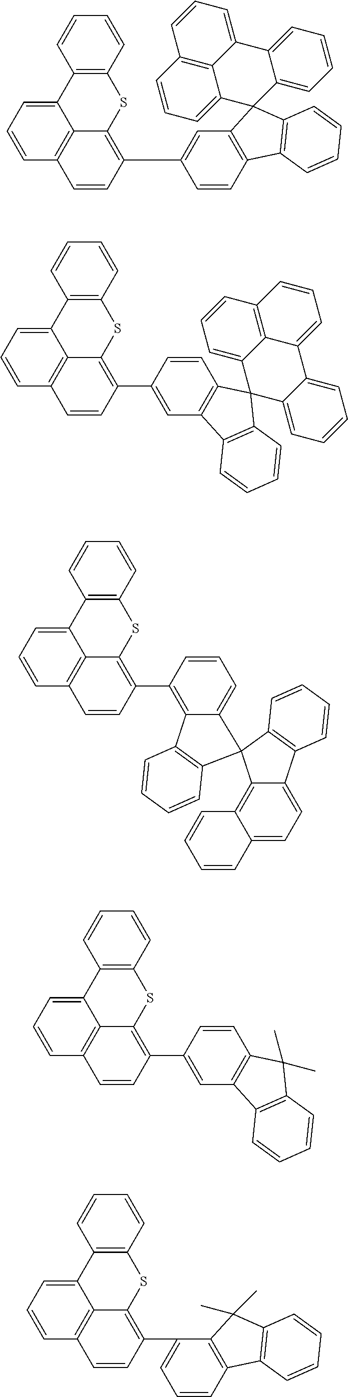 [SMILES: c1ccc2c(c1)Sc1c(-c3ccc4c(c3)C3(c5ccccc5-4)c4ccccc4-c4cccc5cccc3c45)ccc3cccc-2c13 ; c1ccc2c(c1)Sc1c(-c3ccc4c(c3)-c3ccccc3C43c4ccccc4-c4cccc5cccc3c45)ccc3cccc-2c13 ; c1ccc2c(c1)Sc1c(-c3cccc4c3-c3ccccc3C43c4ccccc4-c4ccc5ccccc5c43)ccc3cccc-2c13 ; CC1(C)c2ccccc2-c2cc(-c3ccc4cccc5c4c3Sc3ccccc3-5)ccc21 ; CC1(C)c2ccccc2-c2cccc(-c3ccc4cccc5c4c3Sc3ccccc3-5)c21]

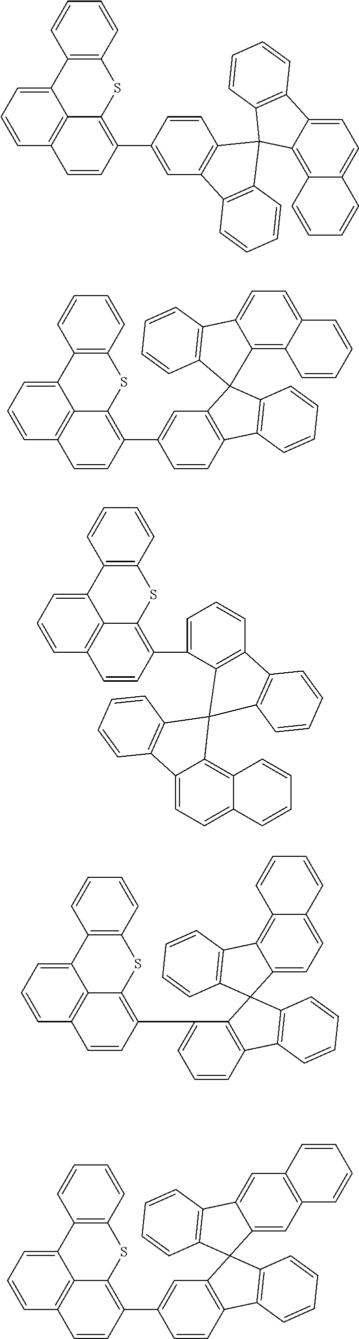

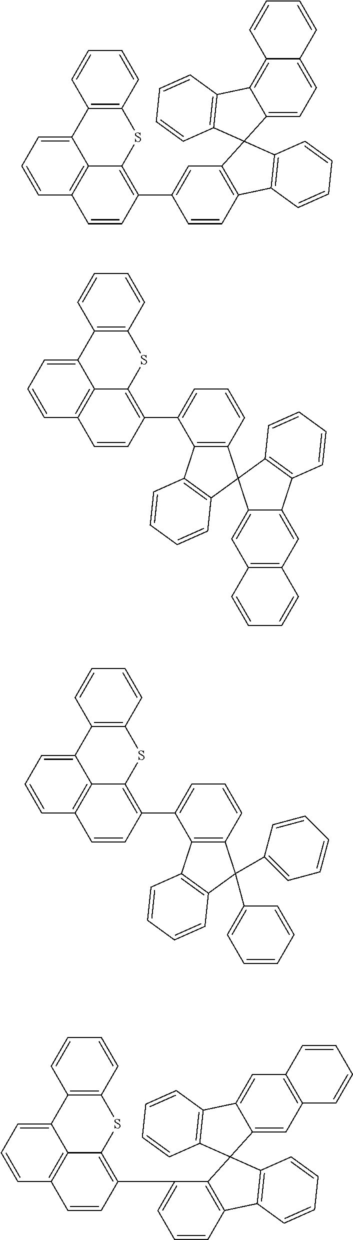 [SMILES: c1ccc2c(c1)Sc1c(-c3ccc4c(c3)C3(c5ccccc5-4)c4ccccc4-c4c3ccc3ccccc43)ccc3cccc-2c13 ; c1ccc2c(c1)Sc1c(-c3cccc4c3-c3ccccc3C43c4ccccc4-c4cc5ccccc5cc43)ccc3cccc-2c13 ; c1ccc(C2(c3ccccc3)c3ccccc3-c3c(-c4ccc5cccc6c5c4Sc4ccccc4-6)cccc32)cc1 ; c1ccc2c(c1)Sc1c(-c3cccc4c3C3(c5ccccc5-c5cc6ccccc6cc53)c3ccccc3-4)ccc3cccc-2c13]

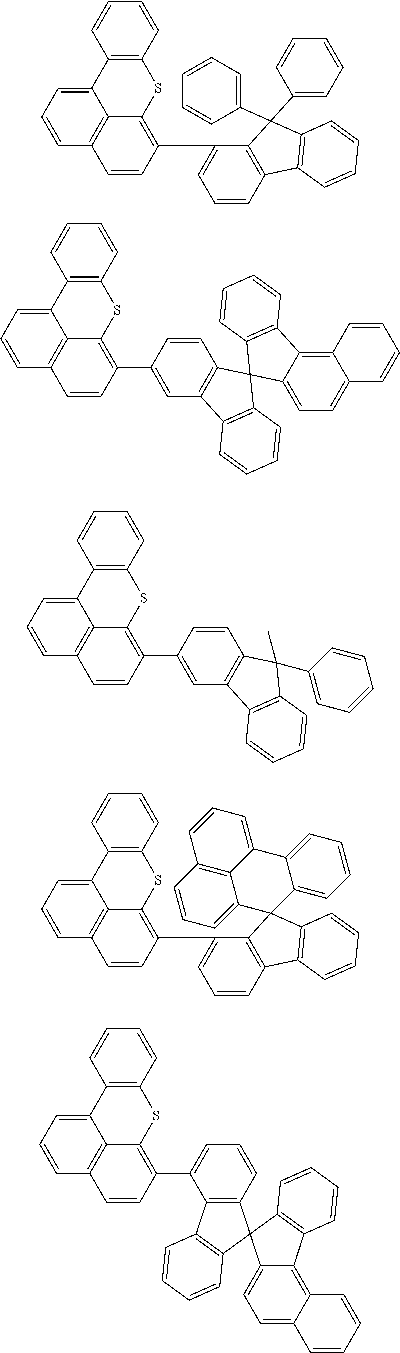 [SMILES: c1ccc(C2(c3ccccc3)c3ccccc3-c3cccc(-c4ccc5cccc6c5c4Sc4ccccc4-6)c32)cc1 ; c1ccc2c(c1)Sc1c(-c3ccc4c(c3)-c3ccccc3C43c4ccccc4-c4c3ccc3ccccc43)ccc3cccc-2c13 ; CC1(c2ccccc2)c2ccccc2-c2cc(-c3ccc4cccc5c4c3Sc3ccccc3-5)ccc21 ; c1ccc2c(c1)Sc1c(-c3cccc4c3C3(c5ccccc5-4)c4ccccc4-c4cccc5cccc3c45)ccc3cccc-2c13 ; c1ccc2c(c1)Sc1c(-c3cccc4c3-c3ccccc3C43c4ccccc4-c4c3ccc3ccccc43)ccc3cccc-2c13]

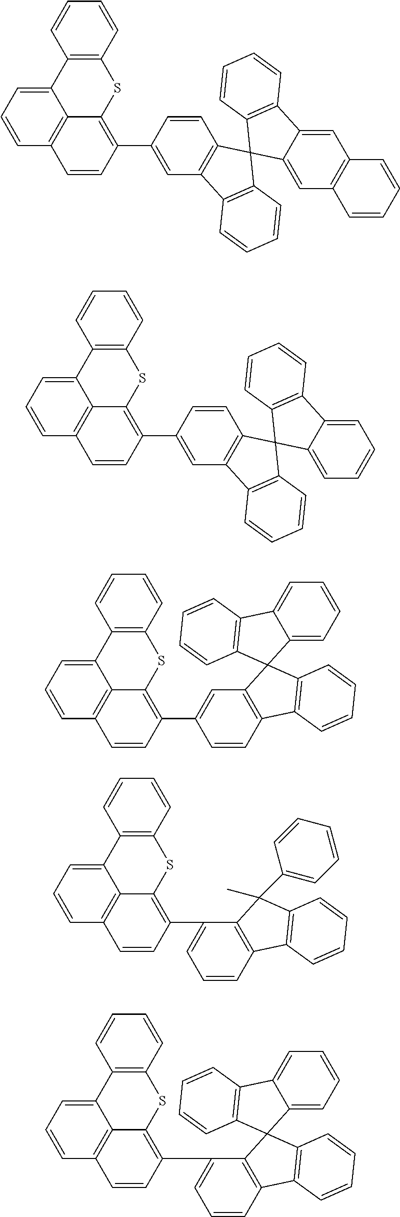

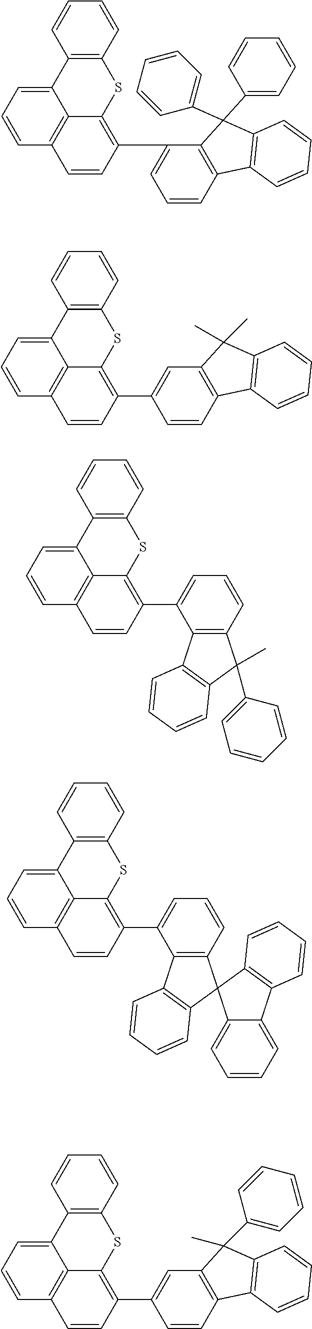

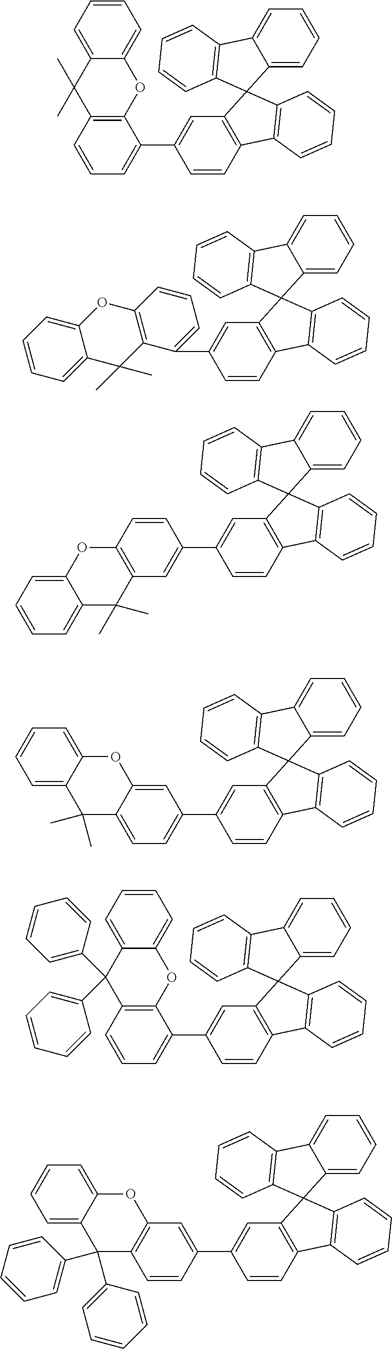 [SMILES: CC1(C)c2ccccc2Oc2c(-c3ccc4c(c3)C3(c5ccccc5-c5ccccc53)c3ccccc3-4)cccc21 ; CC1(C)c2ccccc2Oc2cccc(-c3ccc4c(c3)C3(c5ccccc5-c5ccccc53)c3ccccc3-4)c21 ; CC1(C)c2ccccc2Oc2ccc(-c3ccc4c(c3)C3(c5ccccc5-c5ccccc53)c3ccccc3-4)cc21 ; CC1(C)c2ccccc2Oc2cc(-c3ccc4c(c3)C3(c5ccccc5-c5ccccc53)c3ccccc3-4)ccc21 ; c1ccc(C2(c3ccccc3)c3ccccc3Oc3c(-c4ccc5c(c4)C4(c6ccccc6-c6ccccc64)c4ccccc4-5)cccc32)cc1 ; c1ccc(C2(c3ccccc3)c3ccccc3Oc3cc(-c4ccc5c(c4)C4(c6ccccc6-c6ccccc64)c4ccccc4-5)ccc32)cc1]

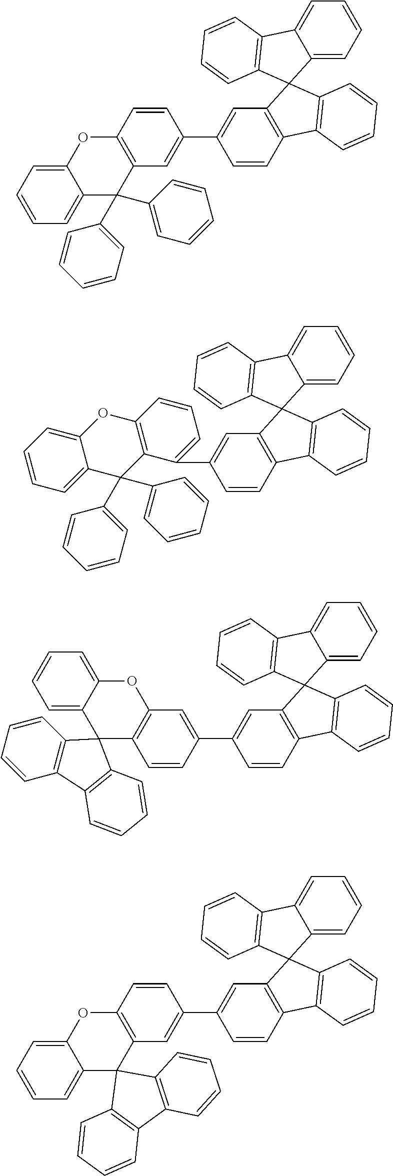 [SMILES: c1ccc(C2(c3ccccc3)c3ccccc3Oc3ccc(-c4ccc5c(c4)C4(c6ccccc6-c6ccccc64)c4ccccc4-5)cc32)cc1 ; c1ccc(C2(c3ccccc3)c3ccccc3Oc3cccc(-c4ccc5c(c4)C4(c6ccccc6-c6ccccc64)c4ccccc4-5)c32)cc1 ; c1ccc2c(c1)Oc1cc(-c3ccc4c(c3)C3(c5ccccc5-c5ccccc53)c3ccccc3-4)ccc1C21c2ccccc2-c2ccccc21 ; c1ccc2c(c1)Oc1ccc(-c3ccc4c(c3)C3(c5ccccc5-c5ccccc53)c3ccccc3-4)cc1C21c2ccccc2-c2ccccc21]

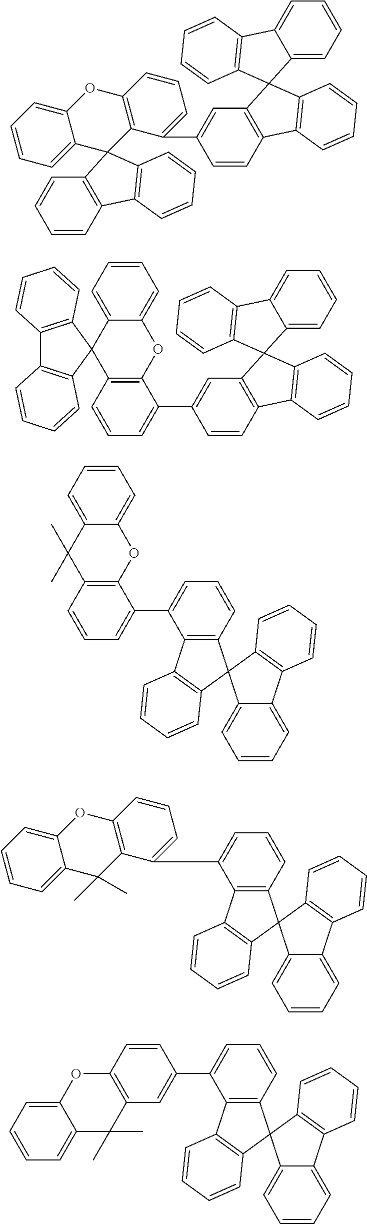

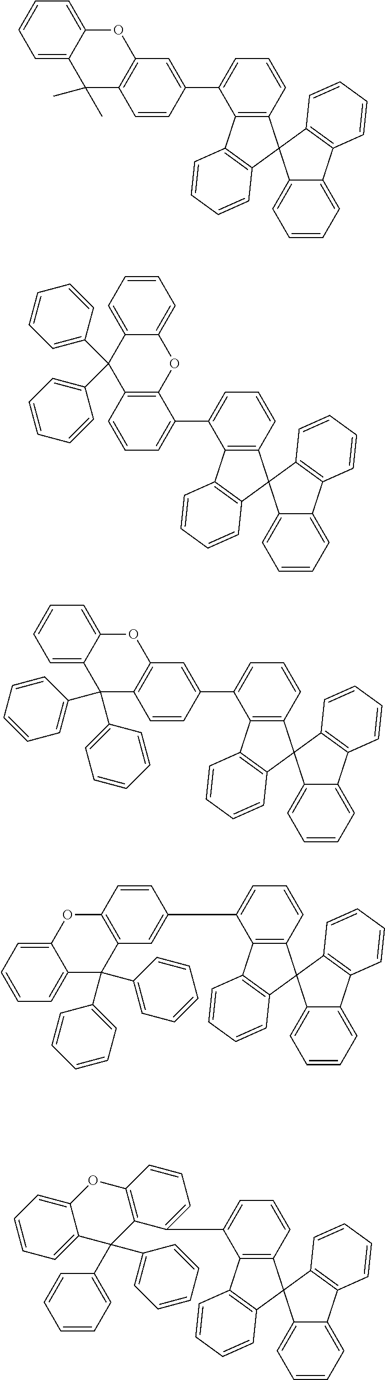 [SMILES: CC1(C)c2ccccc2Oc2cc(-c3cccc4c3-c3ccccc3C43c4ccccc4-c4ccccc43)ccc21 ; c1ccc(C2(c3ccccc3)c3ccccc3Oc3c(-c4cccc5c4-c4ccccc4C54c5ccccc5-c5ccccc54)cccc32)cc1 ; c1ccc(C2(c3ccccc3)c3ccccc3Oc3cc(-c4cccc5c4-c4ccccc4C54c5ccccc5-c5ccccc54)ccc32)cc1 ; c1ccc(C2(c3ccccc3)c3ccccc3Oc3ccc(-c4cccc5c4-c4ccccc4C54c5ccccc5-c5ccccc54)cc32)cc1 ; c1ccc(C2(c3ccccc3)c3ccccc3Oc3cccc(-c4cccc5c4-c4ccccc4C54c5ccccc5-c5ccccc54)c32)cc1]

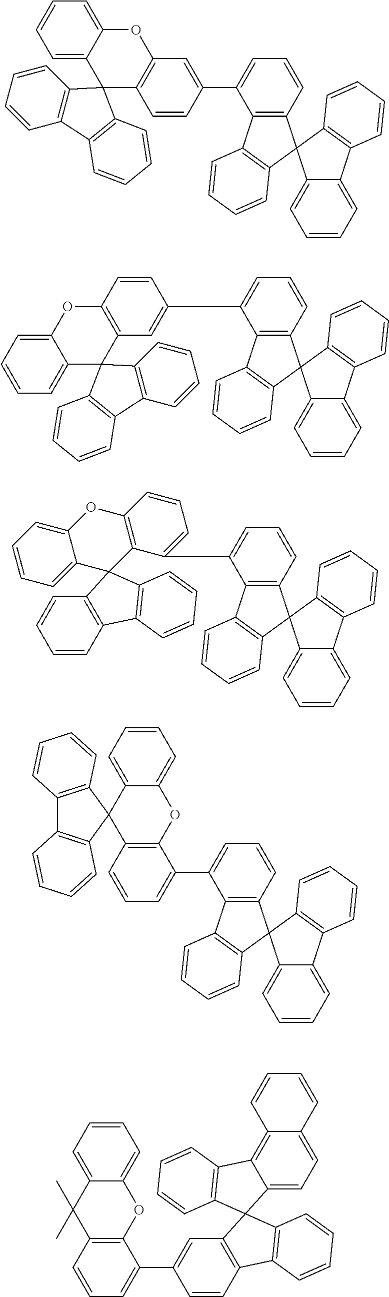

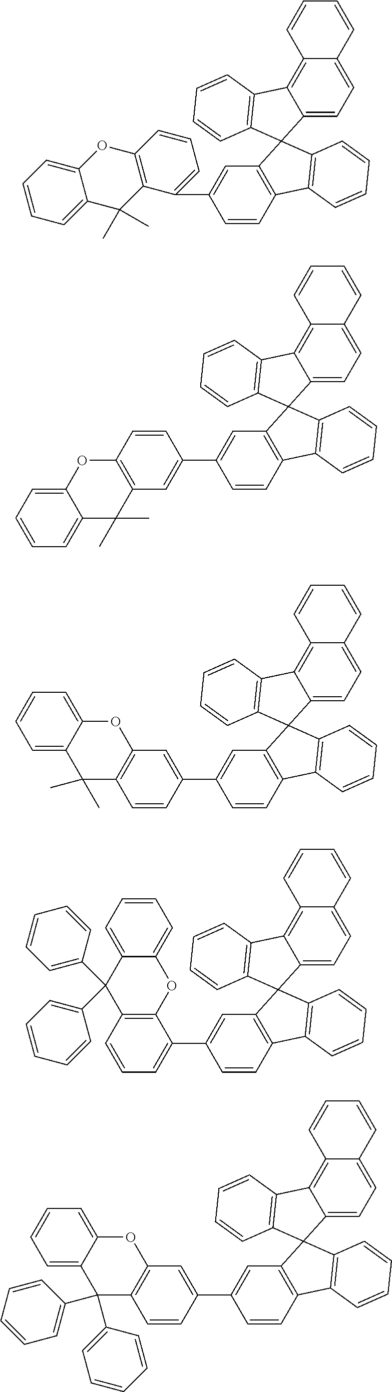

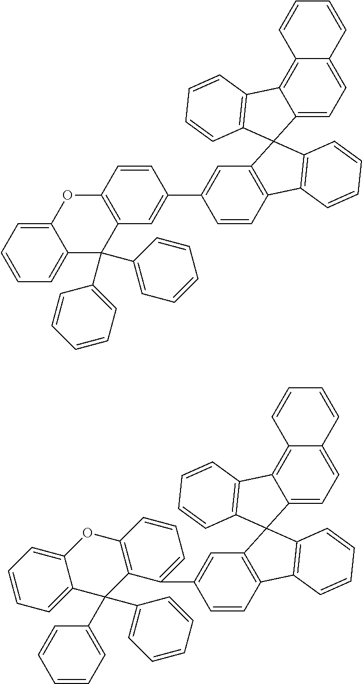 [SMILES: c1ccc(C2(c3ccccc3)c3ccccc3Oc3ccc(-c4ccc5c(c4)C4(c6ccccc6-5)c5ccccc5-c5c4ccc4ccccc54)cc32)cc1 ; c1ccc(C2(c3ccccc3)c3ccccc3Oc3cccc(-c4ccc5c(c4)C4(c6ccccc6-5)c5ccccc5-c5c4ccc4ccccc54)c32)cc1]

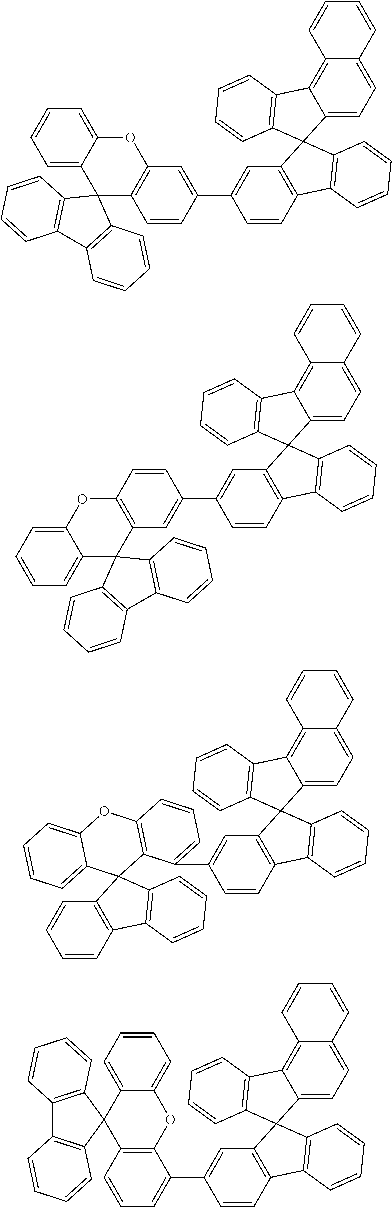 [SMILES: c1ccc2c(c1)Oc1cc(-c3ccc4c(c3)C3(c5ccccc5-4)c4ccccc4-c4c3ccc3ccccc43)ccc1C21c2ccccc2-c2ccccc21 ; c1ccc2c(c1)Oc1ccc(-c3ccc4c(c3)C3(c5ccccc5-4)c4ccccc4-c4c3ccc3ccccc43)cc1C21c2ccccc2-c2ccccc21 ; c1ccc2c(c1)Oc1cccc(-c3ccc4c(c3)C3(c5ccccc5-4)c4ccccc4-c4c3ccc3ccccc43)c1C21c2ccccc2-c2ccccc21 ; c1ccc2c(c1)Oc1c(-c3ccc4c(c3)C3(c5ccccc5-4)c4ccccc4-c4c3ccc3ccccc43)cccc1C21c2ccccc2-c2ccccc21]

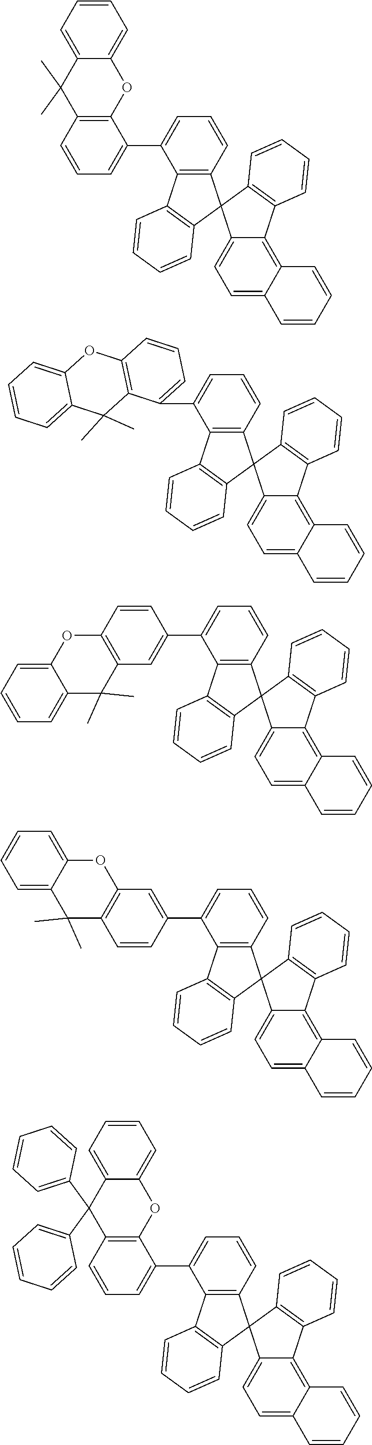 [SMILES: CC1(C)c2ccccc2Oc2c(-c3cccc4c3-c3ccccc3C43c4ccccc4-c4c3ccc3ccccc43)cccc21 ; CC1(C)c2ccccc2Oc2cccc(-c3cccc4c3-c3ccccc3C43c4ccccc4-c4c3ccc3ccccc43)c21 ; CC1(C)c2ccccc2Oc2ccc(-c3cccc4c3-c3ccccc3C43c4ccccc4-c4c3ccc3ccccc43)cc21 ; CC1(C)c2ccccc2Oc2cc(-c3cccc4c3-c3ccccc3C43c4ccccc4-c4c3ccc3ccccc43)ccc21 ; c1ccc(C2(c3ccccc3)c3ccccc3Oc3c(-c4cccc5c4-c4ccccc4C54c5ccccc5-c5c4ccc4ccccc54)cccc32)cc1]

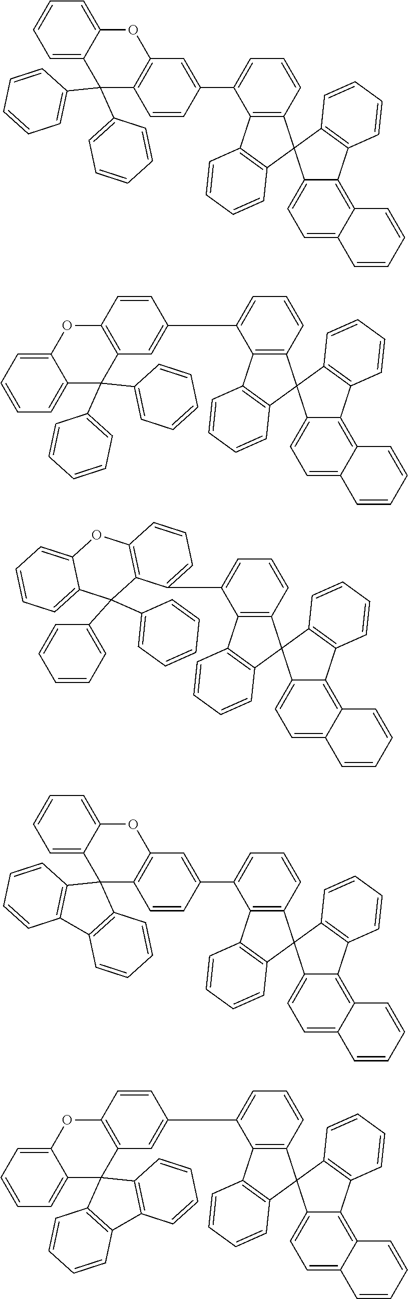 [SMILES: c1ccc(C2(c3ccccc3)c3ccccc3Oc3cc(-c4cccc5c4-c4ccccc4C54c5ccccc5-c5c4ccc4ccccc54)ccc32)cc1 ; c1ccc(C2(c3ccccc3)c3ccccc3Oc3ccc(-c4cccc5c4-c4ccccc4C54c5ccccc5-c5c4ccc4ccccc54)cc32)cc1 ; c1ccc(C2(c3ccccc3)c3ccccc3Oc3cccc(-c4cccc5c4-c4ccccc4C54c5ccccc5-c5c4ccc4ccccc54)c32)cc1 ; c1ccc2c(c1)Oc1cc(-c3cccc4c3-c3ccccc3C43c4ccccc4-c4c3ccc3ccccc43)ccc1C21c2ccccc2-c2ccccc21 ; c1ccc2c(c1)Oc1ccc(-c3cccc4c3-c3ccccc3C43c4ccccc4-c4c3ccc3ccccc43)cc1C21c2ccccc2-c2ccccc21]

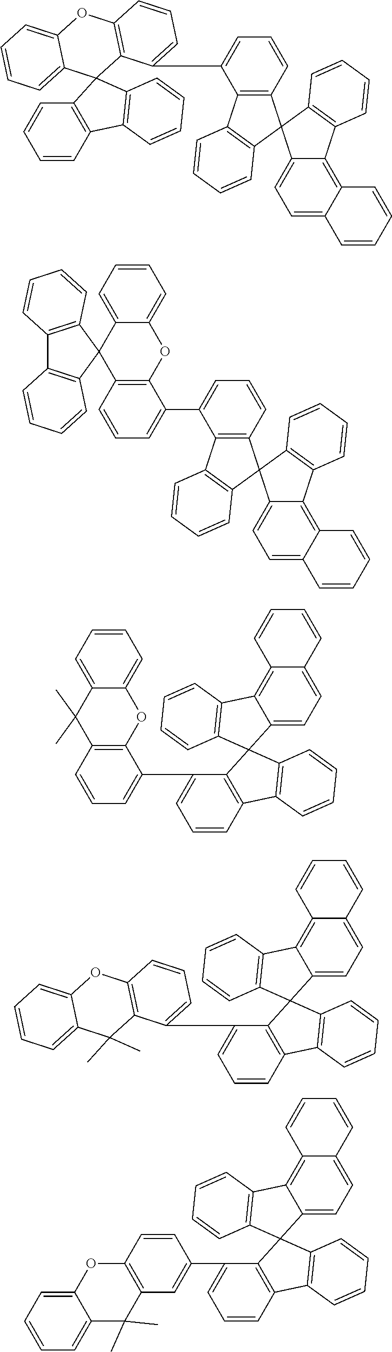

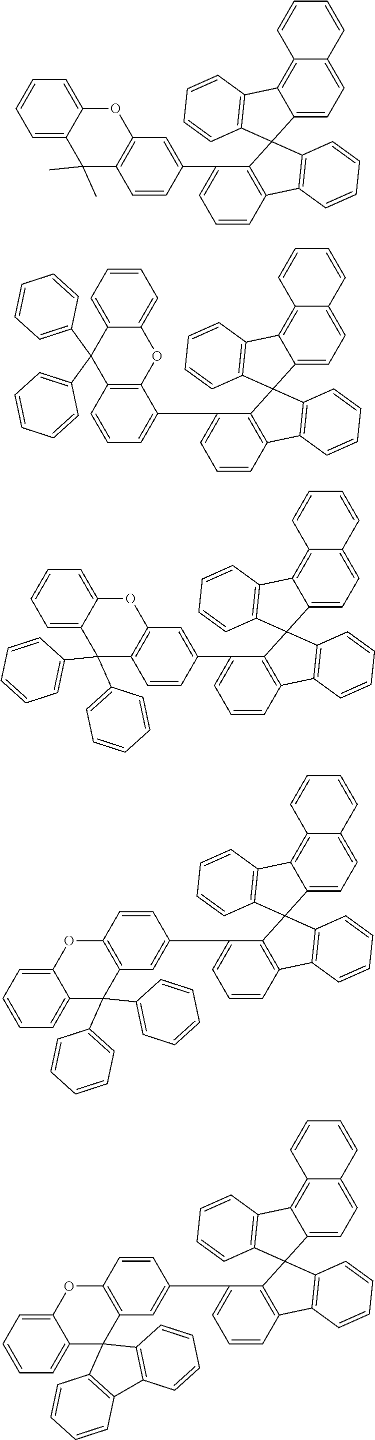

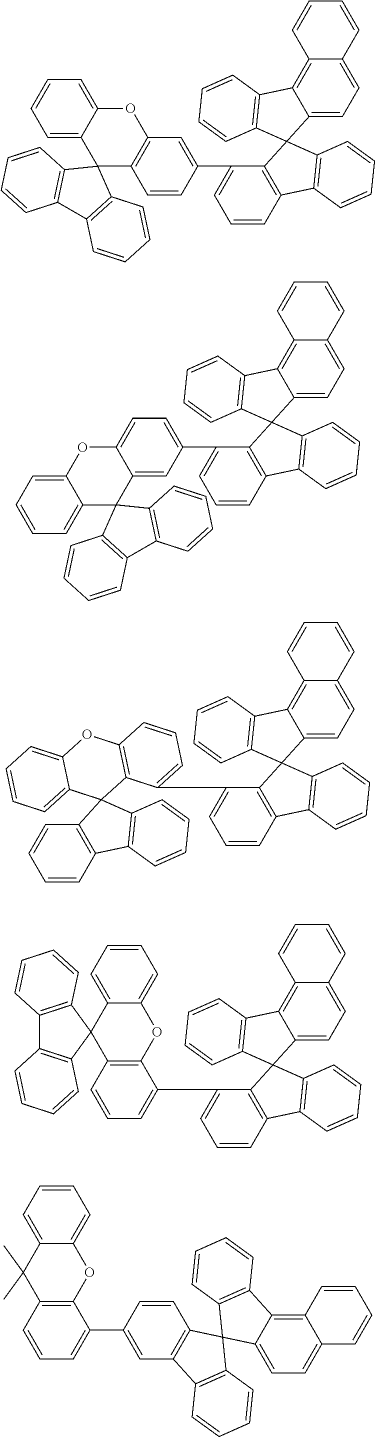 [SMILES: c1ccc2c(c1)Oc1cc(-c3cccc4c3C3(c5ccccc5-4)c4ccccc4-c4c3ccc3ccccc43)ccc1C21c2ccccc2-c2ccccc21 ; c1ccc2c(c1)Oc1ccc(-c3cccc4c3C3(c5ccccc5-4)c4ccccc4-c4c3ccc3ccccc43)cc1C21c2ccccc2-c2ccccc21 ; c1ccc2c(c1)Oc1cccc(-c3cccc4c3C3(c5ccccc5-4)c4ccccc4-c4c3ccc3ccccc43)c1C21c2ccccc2-c2ccccc21 ; c1ccc2c(c1)Oc1c(-c3cccc4c3C3(c5ccccc5-4)c4ccccc4-c4c3ccc3ccccc43)cccc1C21c2ccccc2-c2ccccc21 ; CC1(C)c2ccccc2Oc2c(-c3ccc4c(c3)-c3ccccc3C43c4ccccc4-c4c3ccc3ccccc43)cccc21]

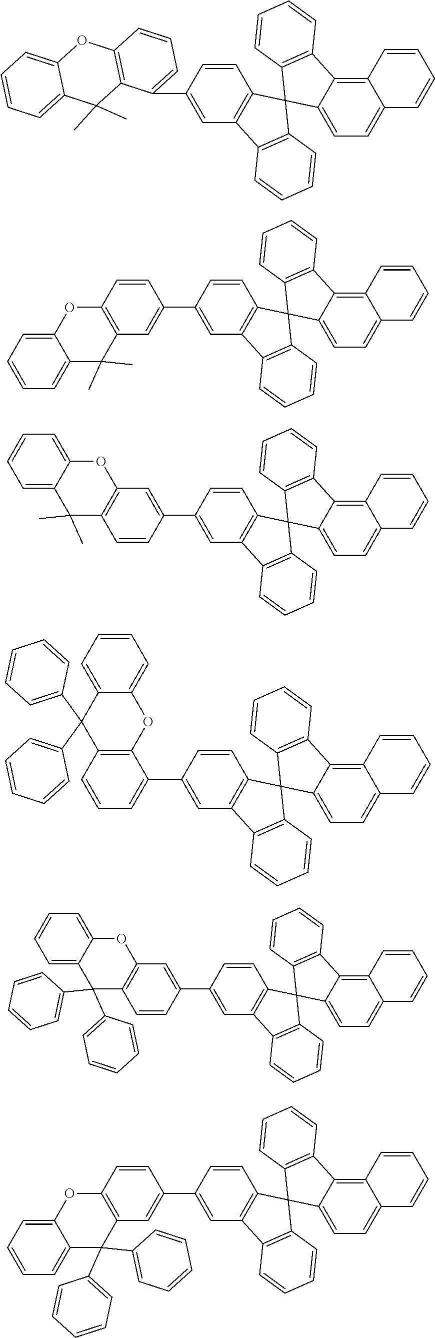

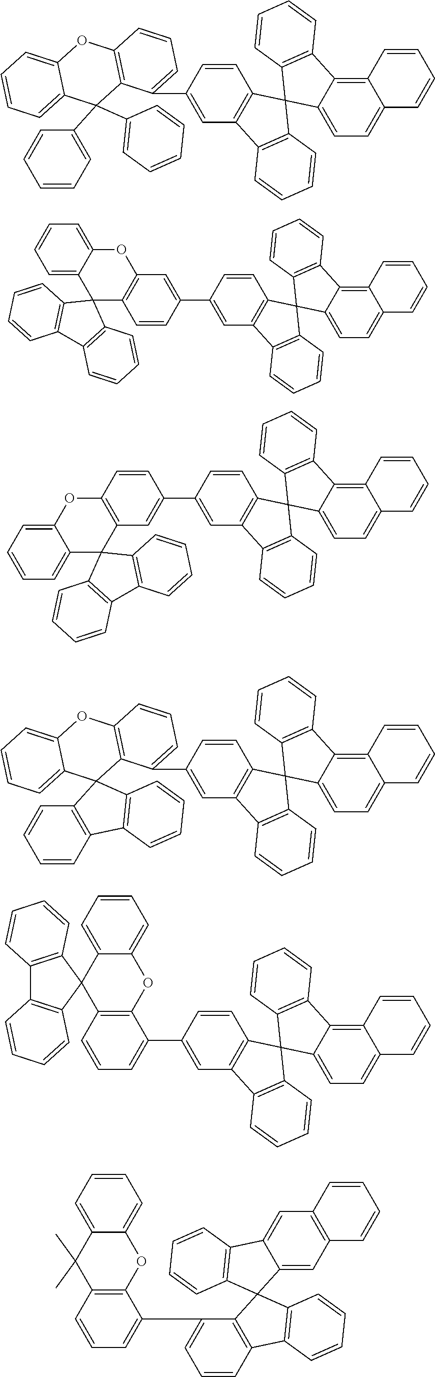

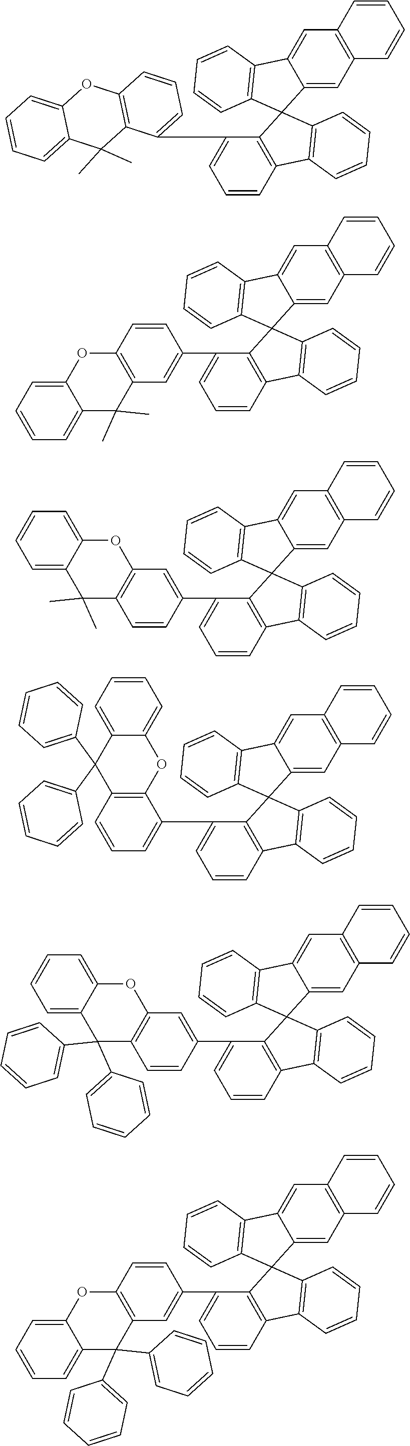

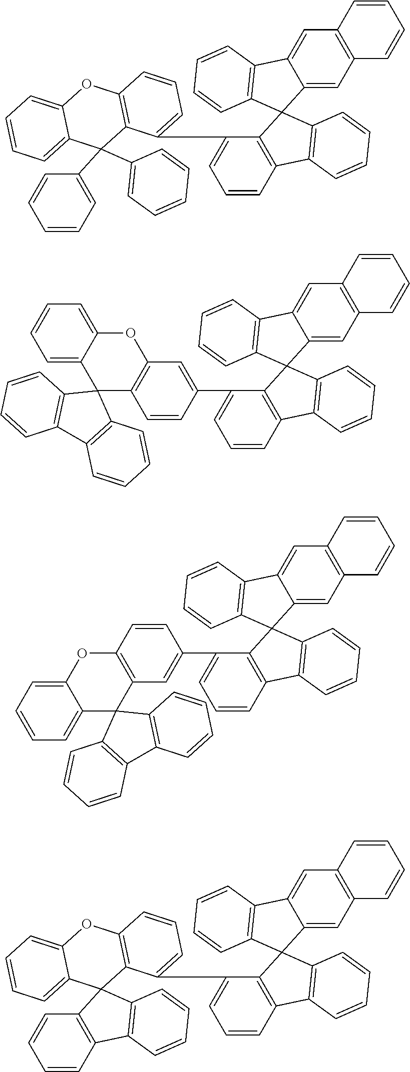 [SMILES: c1ccc(C2(c3ccccc3)c3ccccc3Oc3cccc(-c4cccc5c4C4(c6ccccc6-c6cc7ccccc7cc64)c4ccccc4-5)c32)cc1 ; c1ccc2c(c1)Oc1cc(-c3cccc4c3C3(c5ccccc5-c5cc6ccccc6cc53)c3ccccc3-4)ccc1C21c2ccccc2-c2ccccc21 ; c1ccc2c(c1)Oc1ccc(-c3cccc4c3C3(c5ccccc5-c5cc6ccccc6cc53)c3ccccc3-4)cc1C21c2ccccc2-c2ccccc21 ; c1ccc2c(c1)Oc1cccc(-c3cccc4c3C3(c5ccccc5-c5cc6ccccc6cc53)c3ccccc3-4)c1C21c2ccccc2-c2ccccc21]

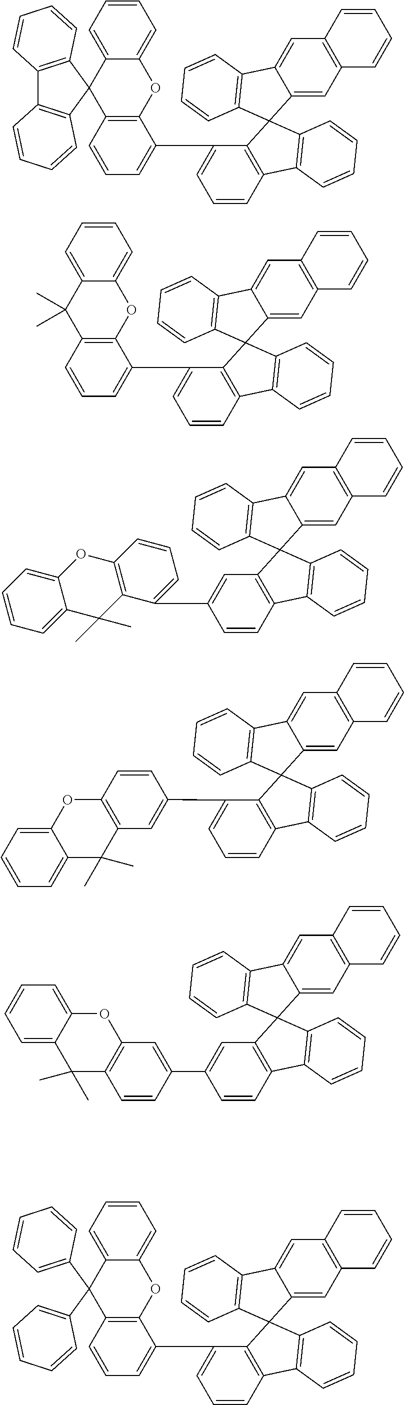

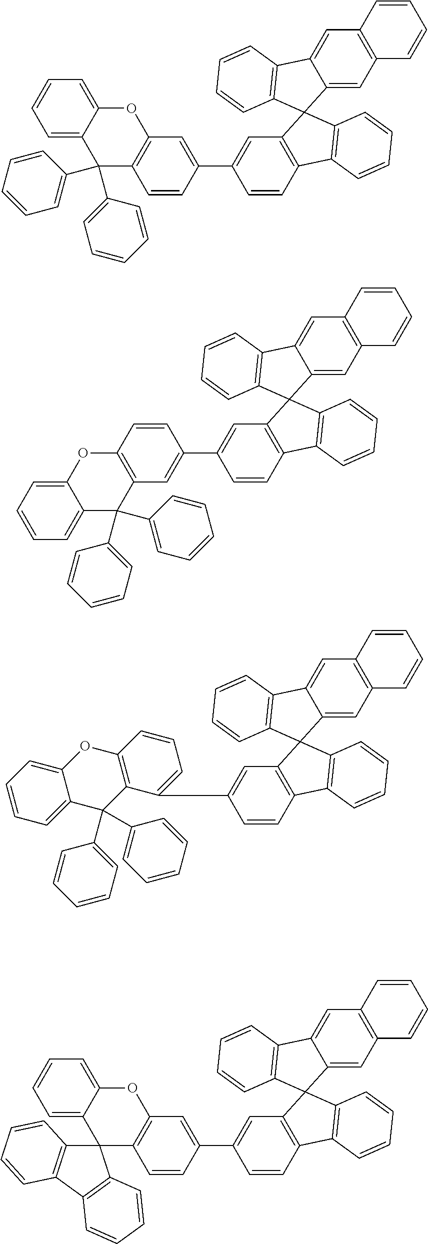 [SMILES: c1ccc(C2(c3ccccc3)c3ccccc3Oc3cc(-c4ccc5c(c4)C4(c6ccccc6-5)c5ccccc5-c5cc6ccccc6cc54)ccc32)cc1 ; c1ccc(C2(c3ccccc3)c3ccccc3Oc3ccc(-c4ccc5c(c4)C4(c6ccccc6-5)c5ccccc5-c5cc6ccccc6cc54)cc32)cc1 ; c1ccc(C2(c3ccccc3)c3ccccc3Oc3cccc(-c4ccc5c(c4)C4(c6ccccc6-5)c5ccccc5-c5cc6ccccc6cc54)c32)cc1 ; c1ccc2c(c1)Oc1cc(-c3ccc4c(c3)C3(c5ccccc5-4)c4ccccc4-c4cc5ccccc5cc43)ccc1C21c2ccccc2-c2ccccc21]

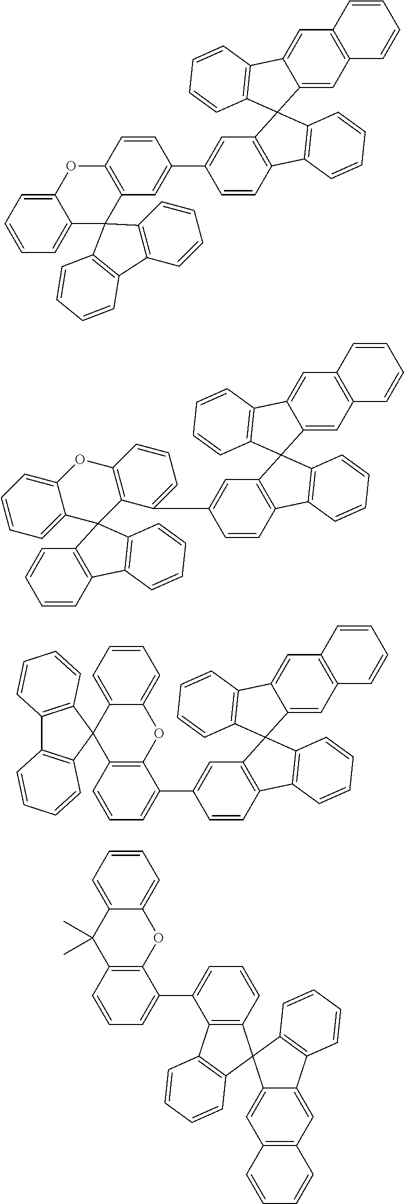

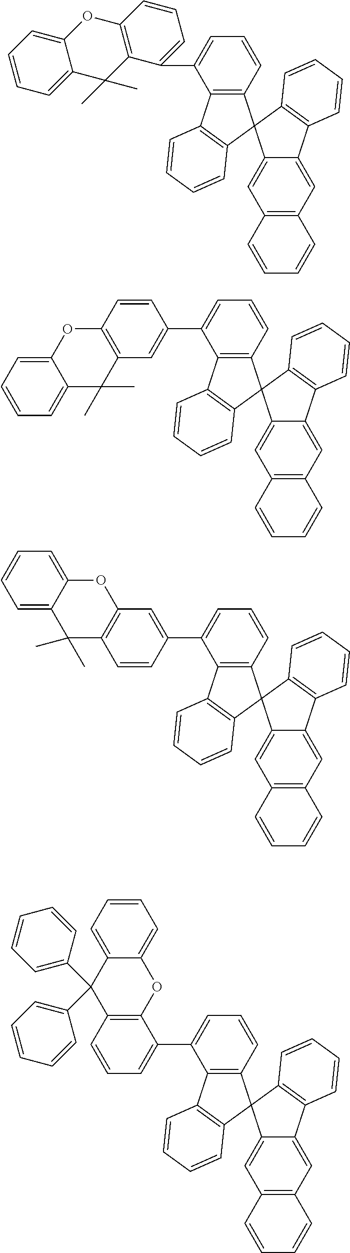

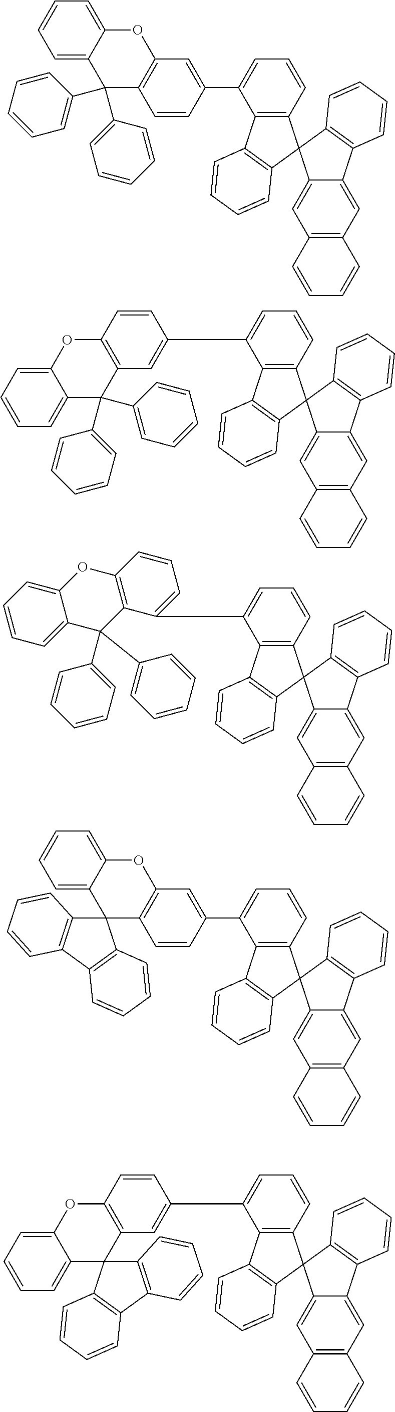

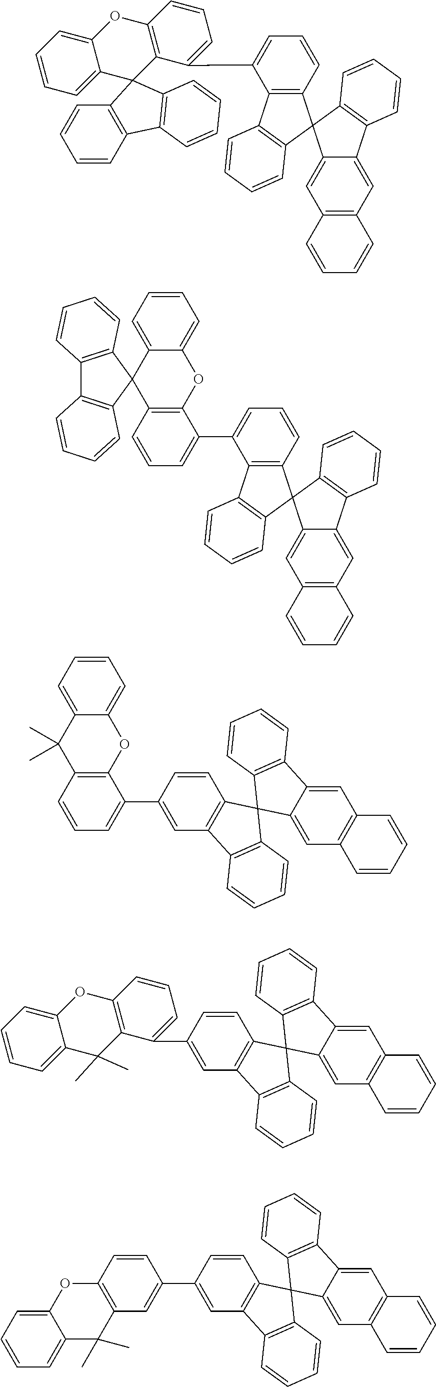 [SMILES: c1ccc2c(c1)Oc1cccc(-c3cccc4c3-c3ccccc3C43c4ccccc4-c4cc5ccccc5cc43)c1C21c2ccccc2-c2ccccc21 ; c1ccc2c(c1)Oc1c(-c3cccc4c3-c3ccccc3C43c4ccccc4-c4cc5ccccc5cc43)cccc1C21c2ccccc2-c2ccccc21 ; CC1(C)c2ccccc2Oc2c(-c3ccc4c(c3)-c3ccccc3C43c4ccccc4-c4cc5ccccc5cc43)cccc21 ; CC1(C)c2ccccc2Oc2cccc(-c3ccc4c(c3)-c3ccccc3C43c4ccccc4-c4cc5ccccc5cc43)c21 ; CC1(C)c2ccccc2Oc2ccc(-c3ccc4c(c3)-c3ccccc3C43c4ccccc4-c4cc5ccccc5cc43)cc21]

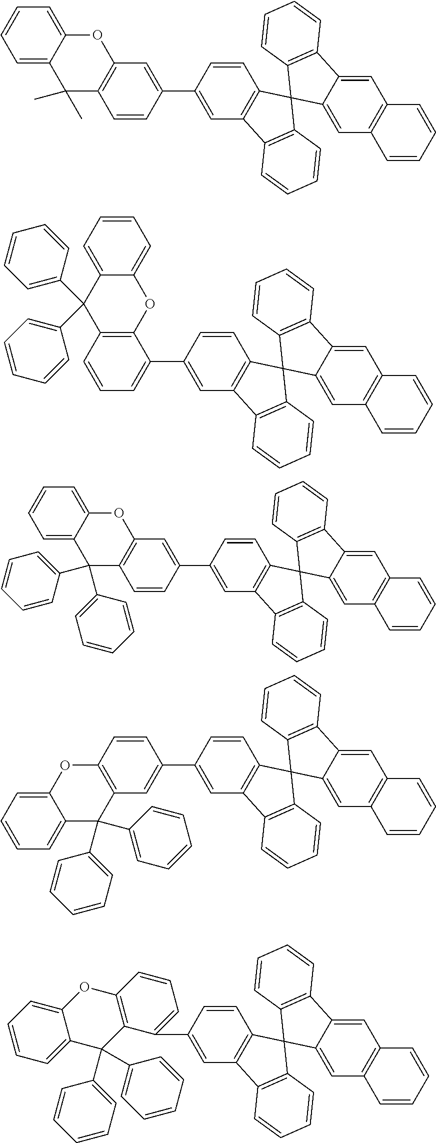

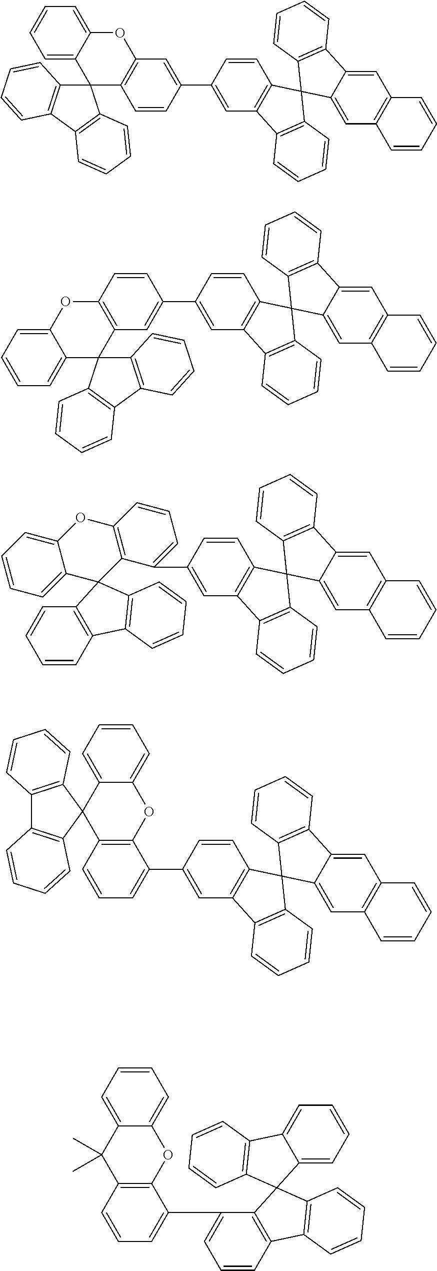

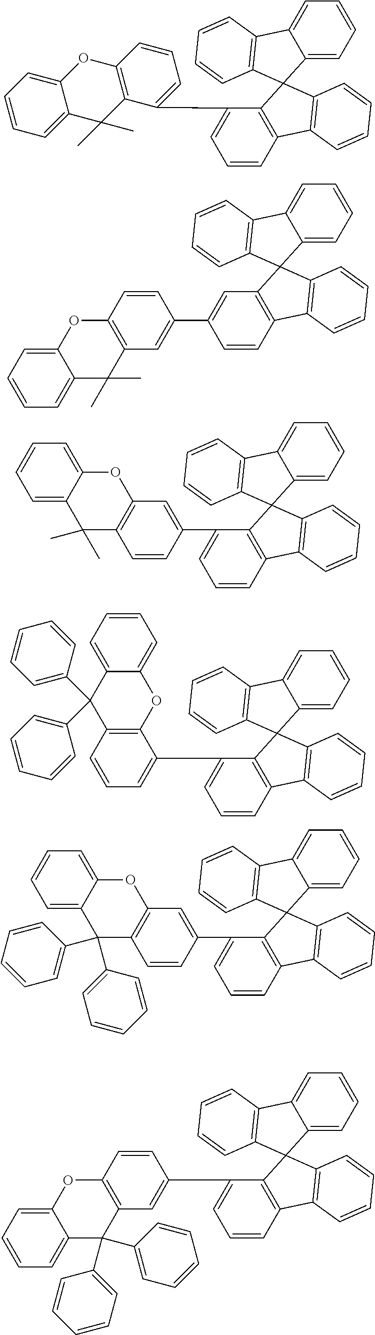

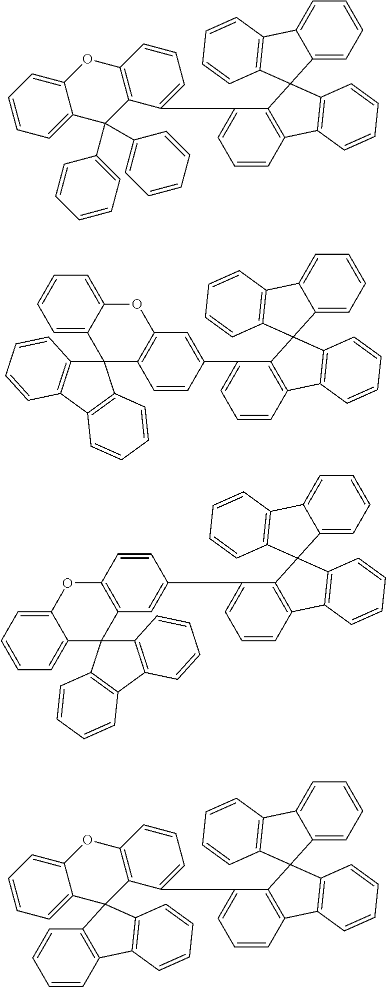 [SMILES: c1ccc(C2(c3ccccc3)c3ccccc3Oc3cccc(-c4cccc5c4C4(c6ccccc6-c6ccccc64)c4ccccc4-5)c32)cc1 ; c1ccc2c(c1)Oc1cc(-c3cccc4c3C3(c5ccccc5-c5ccccc53)c3ccccc3-4)ccc1C21c2ccccc2-c2ccccc21 ; c1ccc2c(c1)Oc1ccc(-c3cccc4c3C3(c5ccccc5-c5ccccc53)c3ccccc3-4)cc1C21c2ccccc2-c2ccccc21 ; c1ccc2c(c1)Oc1cccc(-c3cccc4c3C3(c5ccccc5-c5ccccc53)c3ccccc3-4)c1C21c2ccccc2-c2ccccc21]

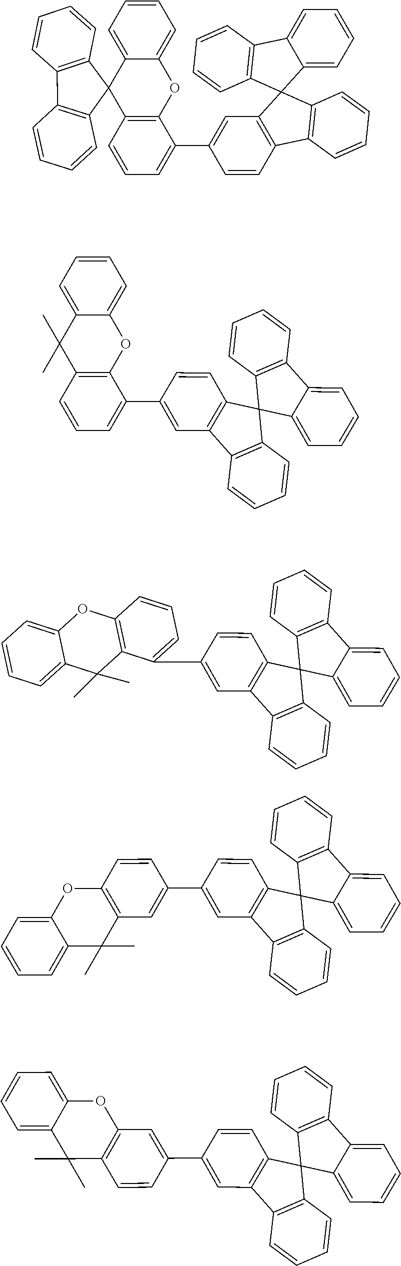

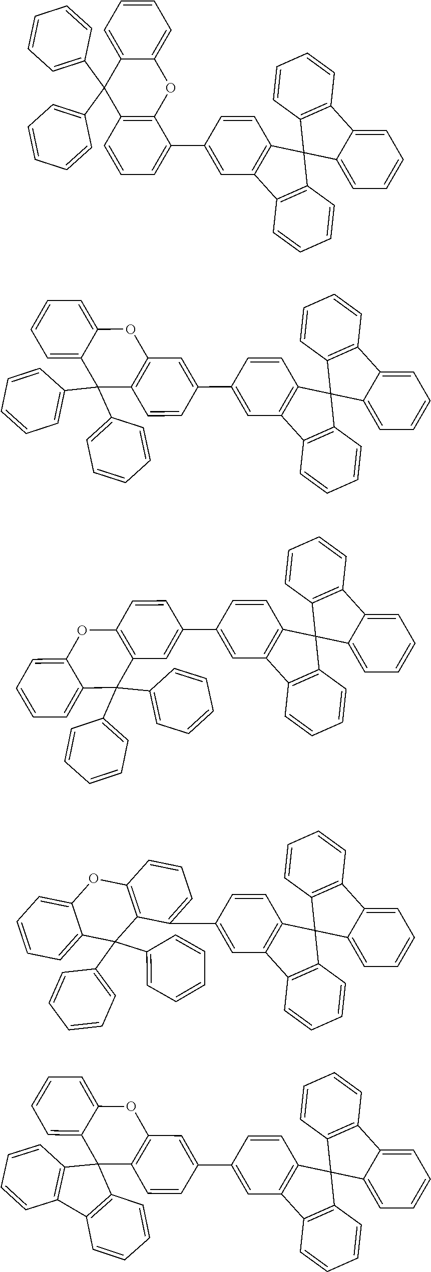

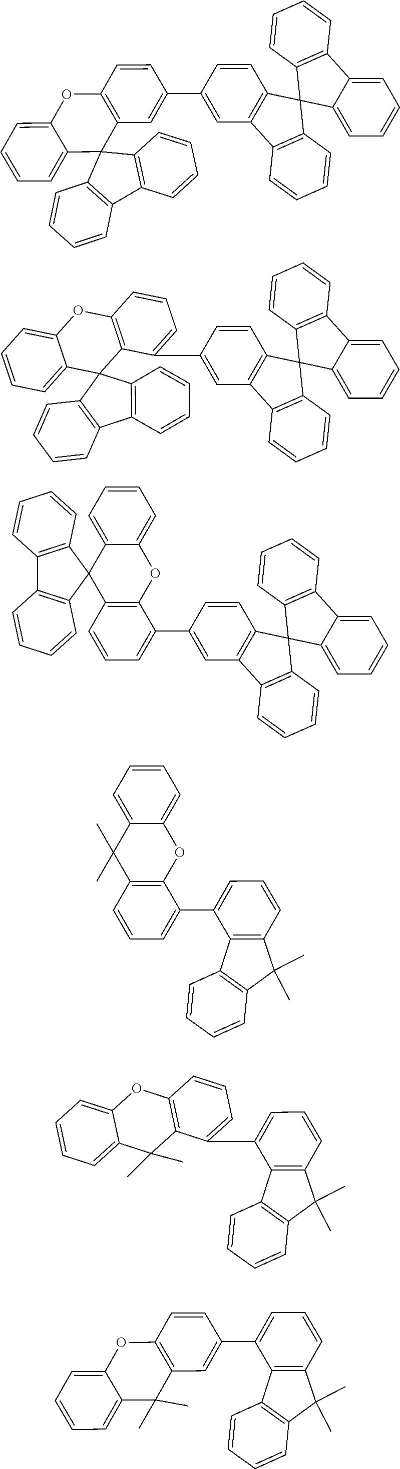

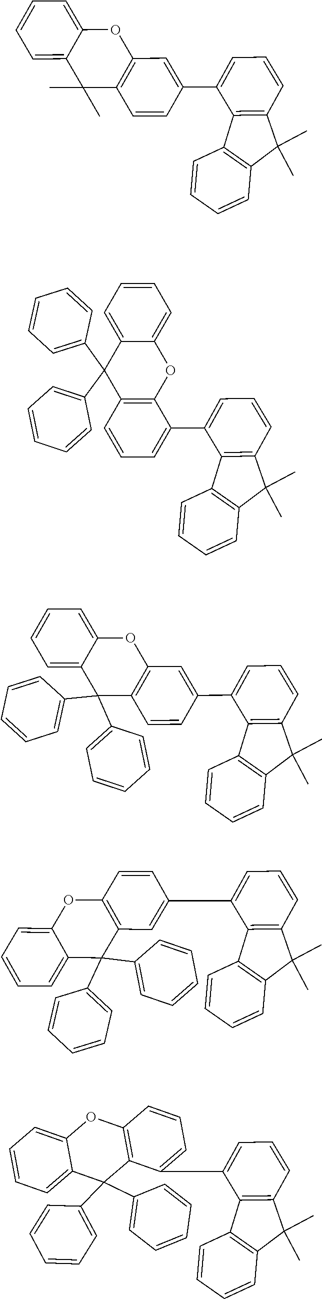 [SMILES: CC1(C)c2ccccc2Oc2cc(-c3cccc4c3-c3ccccc3C4(C)C)ccc21 ; CC1(C)c2ccccc2-c2c(-c3cccc4c3Oc3ccccc3C4(c3ccccc3)c3ccccc3)cccc21 ; CC1(C)c2ccccc2-c2c(-c3ccc4c(c3)Oc3ccccc3C4(c3ccccc3)c3ccccc3)cccc21 ; CC1(C)c2ccccc2-c2c(-c3ccc4c(c3)C(c3ccccc3)(c3ccccc3)c3ccccc3O4)cccc21 ; CC1(C)c2ccccc2-c2c(-c3cccc4c3C(c3ccccc3)(c3ccccc3)c3ccccc3O4)cccc21]

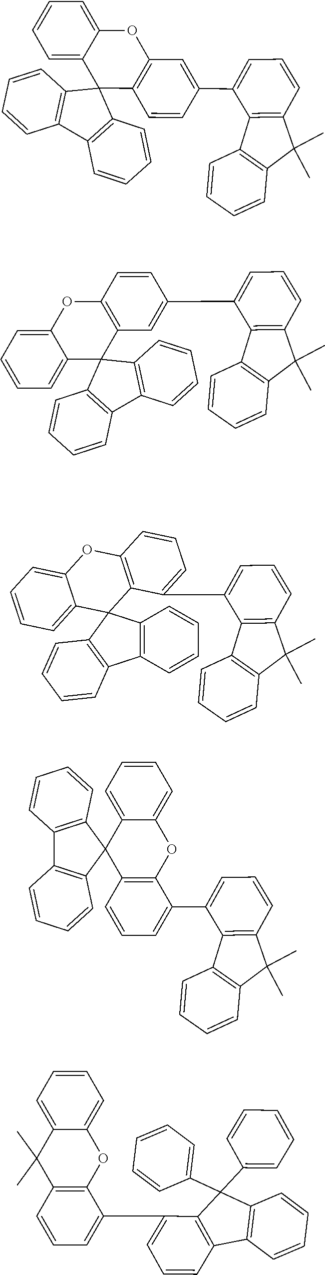

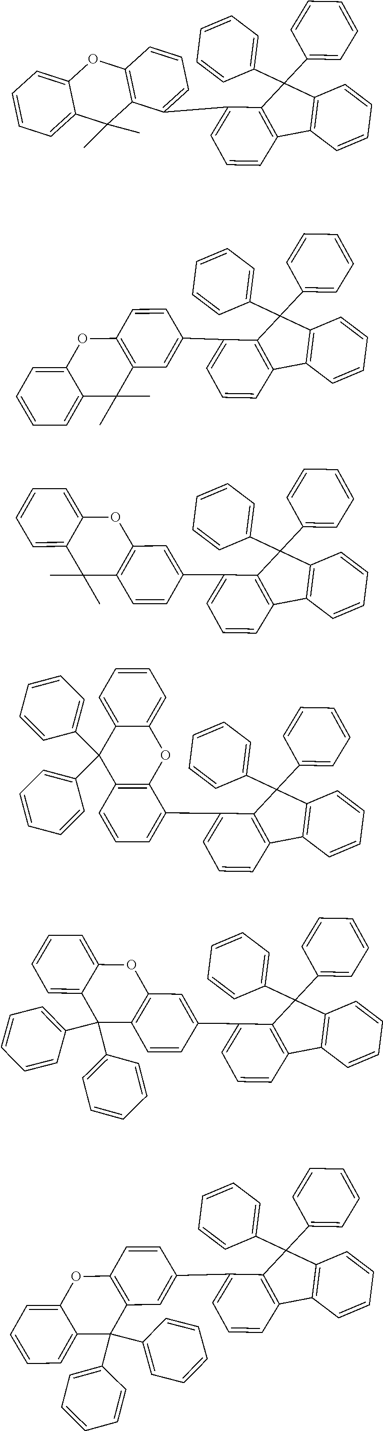 [SMILES: CC1(C)c2ccccc2Oc2cccc(-c3cccc4c3C(c3ccccc3)(c3ccccc3)c3ccccc3-4)c21 ; CC1(C)c2ccccc2Oc2ccc(-c3cccc4c3C(c3ccccc3)(c3ccccc3)c3ccccc3-4)cc21 ; CC1(C)c2ccccc2Oc2cc(-c3cccc4c3C(c3ccccc3)(c3ccccc3)c3ccccc3-4)ccc21 ; c1ccc(C2(c3ccccc3)c3ccccc3Oc3c(-c4cccc5c4C(c4ccccc4)(c4ccccc4)c4ccccc4-5)cccc32)cc1 ; c1ccc(C2(c3ccccc3)c3ccccc3Oc3cc(-c4cccc5c4C(c4ccccc4)(c4ccccc4)c4ccccc4-5)ccc32)cc1 ; c1ccc(C2(c3ccccc3)c3ccccc3Oc3ccc(-c4cccc5c4C(c4ccccc4)(c4ccccc4)c4ccccc4-5)cc32)cc1]

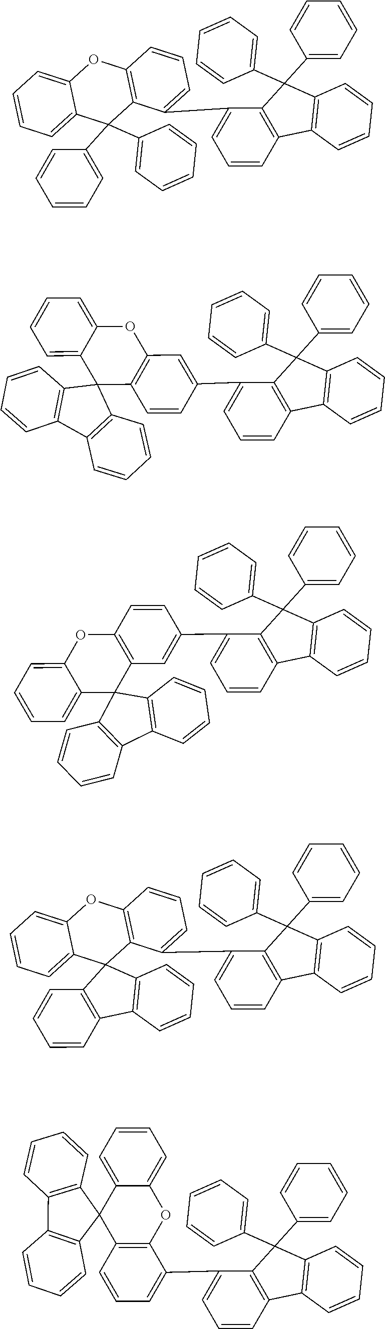 [SMILES: c1ccc(C2(c3ccccc3)c3ccccc3Oc3cccc(-c4cccc5c4C(c4ccccc4)(c4ccccc4)c4ccccc4-5)c32)cc1 ; c1ccc(C2(c3ccccc3)c3ccccc3-c3cccc(-c4ccc5c(c4)Oc4ccccc4C54c5ccccc5-c5ccccc54)c32)cc1 ; c1ccc(C2(c3ccccc3)c3ccccc3-c3cccc(-c4ccc5c(c4)C4(c6ccccc6O5)c5ccccc5-c5ccccc54)c32)cc1 ; c1ccc(C2(c3ccccc3)c3ccccc3-c3cccc(-c4cccc5c4C4(c6ccccc6O5)c5ccccc5-c5ccccc54)c32)cc1 ; c1ccc(C2(c3ccccc3)c3ccccc3-c3cccc(-c4cccc5c4Oc4ccccc4C54c5ccccc5-c5ccccc54)c32)cc1]

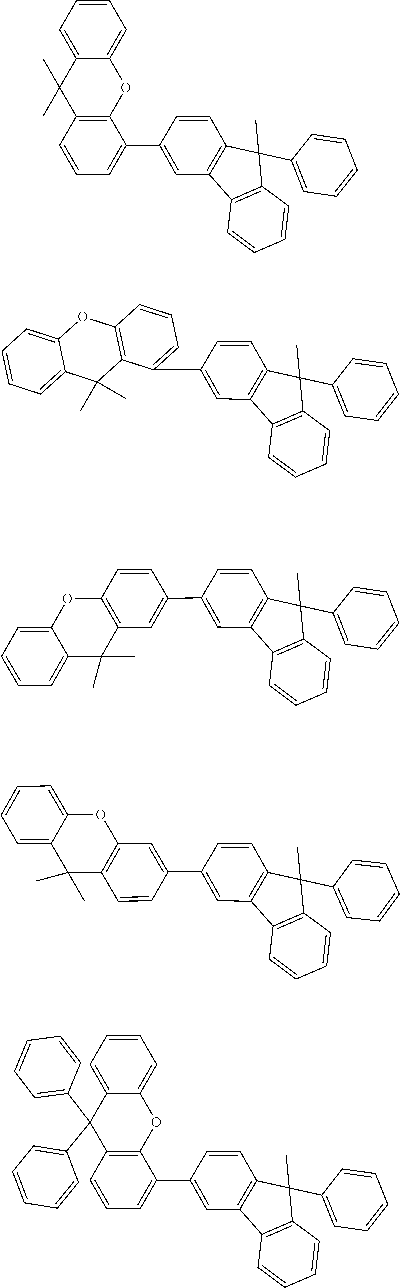 [SMILES: CC1(C)c2ccccc2Oc2c(-c3ccc4c(c3)-c3ccccc3C4(C)c3ccccc3)cccc21 ; CC1(C)c2ccccc2Oc2cccc(-c3ccc4c(c3)-c3ccccc3C4(C)c3ccccc3)c21 ; CC1(C)c2ccccc2Oc2ccc(-c3ccc4c(c3)-c3ccccc3C4(C)c3ccccc3)cc21 ; CC1(C)c2ccccc2Oc2cc(-c3ccc4c(c3)-c3ccccc3C4(C)c3ccccc3)ccc21 ; CC1(c2ccccc2)c2ccccc2-c2cc(-c3cccc4c3Oc3ccccc3C4(c3ccccc3)c3ccccc3)ccc21]

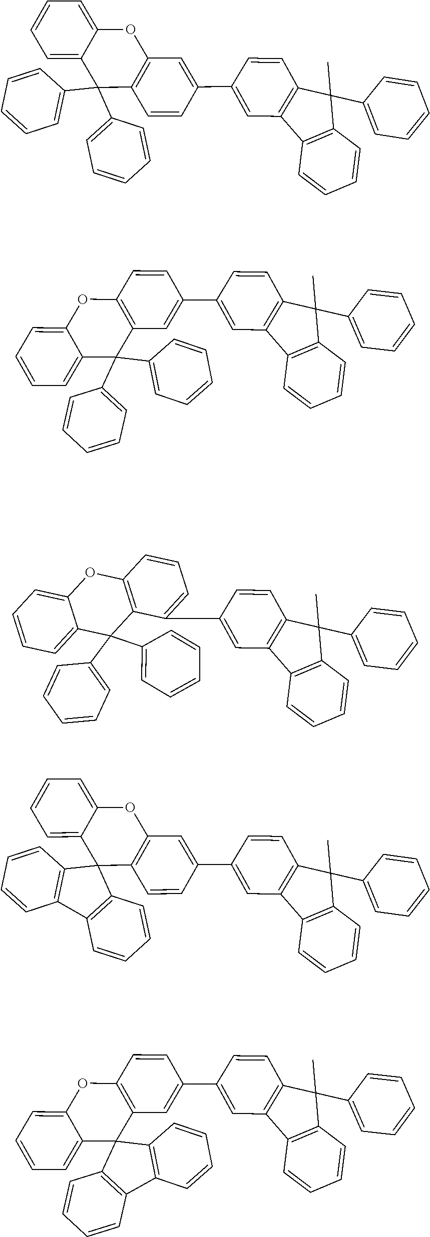 [SMILES: CC1(c2ccccc2)c2ccccc2-c2cc(-c3ccc4c(c3)Oc3ccccc3C4(c3ccccc3)c3ccccc3)ccc21 ; CC1(c2ccccc2)c2ccccc2-c2cc(-c3ccc4c(c3)C(c3ccccc3)(c3ccccc3)c3ccccc3O4)ccc21 ; CC1(c2ccccc2)c2ccccc2-c2cc(-c3cccc4c3C(c3ccccc3)(c3ccccc3)c3ccccc3O4)ccc21 ; CC1(c2ccccc2)c2ccccc2-c2cc(-c3ccc4c(c3)Oc3ccccc3C43c4ccccc4-c4ccccc43)ccc21 ; CC1(c2ccccc2)c2ccccc2-c2cc(-c3ccc4c(c3)C3(c5ccccc5O4)c4ccccc4-c4ccccc43)ccc21]

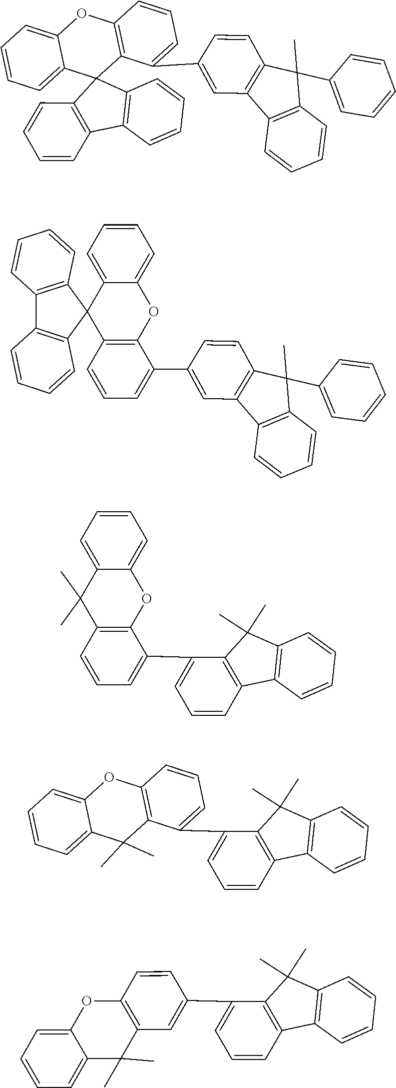 [SMILES: CC1(c2ccccc2)c2ccccc2-c2cc(-c3cccc4c3C3(c5ccccc5O4)c4ccccc4-c4ccccc43)ccc21 ; CC1(c2ccccc2)c2ccccc2-c2cc(-c3cccc4c3Oc3ccccc3C43c4ccccc4-c4ccccc43)ccc21 ; CC1(C)c2ccccc2Oc2c(-c3cccc4c3C(C)(C)c3ccccc3-4)cccc21 ; CC1(C)c2ccccc2Oc2cccc(-c3cccc4c3C(C)(C)c3ccccc3-4)c21 ; CC1(C)c2ccccc2Oc2ccc(-c3cccc4c3C(C)(C)c3ccccc3-4)cc21]

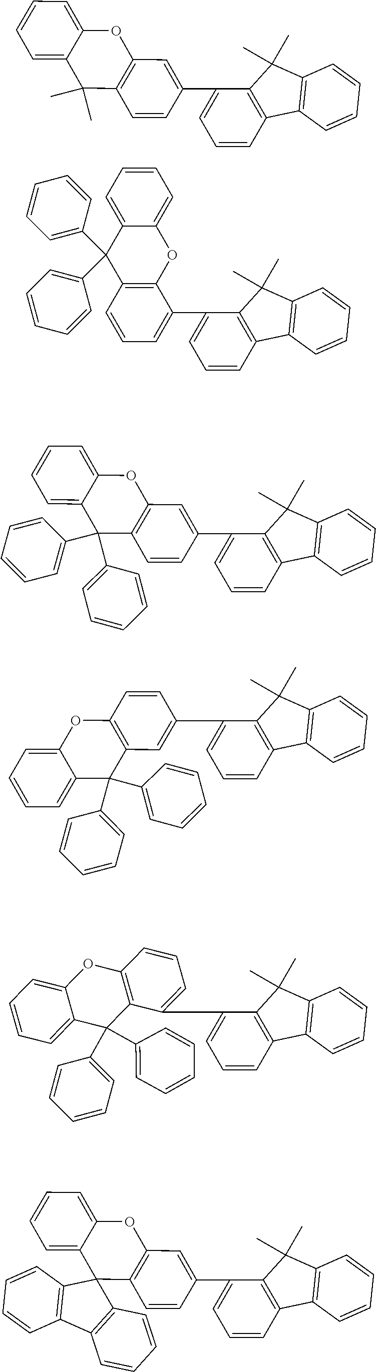 [SMILES: CC1(C)c2ccccc2Oc2cc(-c3cccc4c3C(C)(C)c3ccccc3-4)ccc21 ; CC1(C)c2ccccc2-c2cccc(-c3cccc4c3Oc3ccccc3C4(c3ccccc3)c3ccccc3)c21 ; CC1(C)c2ccccc2-c2cccc(-c3ccc4c(c3)Oc3ccccc3C4(c3ccccc3)c3ccccc3)c21 ; CC1(C)c2ccccc2-c2cccc(-c3ccc4c(c3)C(c3ccccc3)(c3ccccc3)c3ccccc3O4)c21 ; CC1(C)c2ccccc2-c2cccc(-c3cccc4c3C(c3ccccc3)(c3ccccc3)c3ccccc3O4)c21 ; CC1(C)c2ccccc2-c2cccc(-c3ccc4c(c3)Oc3ccccc3C43c4ccccc4-c4ccccc43)c21]

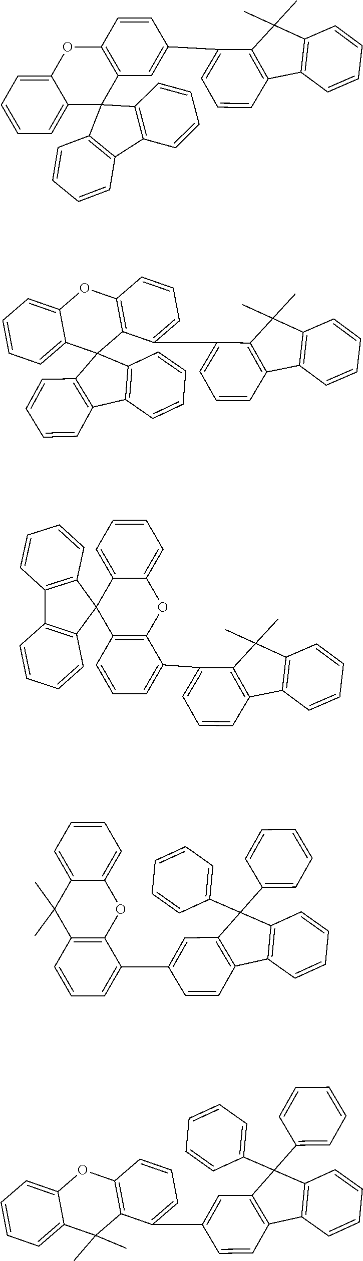 [SMILES: CC1(C)c2ccccc2-c2cccc(-c3ccc4c(c3)C3(c5ccccc5O4)c4ccccc4-c4ccccc43)c21 ; CC1(C)c2ccccc2-c2cccc(-c3cccc4c3C3(c5ccccc5O4)c4ccccc4-c4ccccc43)c21 ; CC1(C)c2ccccc2-c2cccc(-c3cccc4c3Oc3ccccc3C43c4ccccc4-c4ccccc43)c21 ; CC1(C)c2ccccc2Oc2c(-c3ccc4c(c3)C(c3ccccc3)(c3ccccc3)c3ccccc3-4)cccc21 ; CC1(C)c2ccccc2Oc2cccc(-c3ccc4c(c3)C(c3ccccc3)(c3ccccc3)c3ccccc3-4)c21]

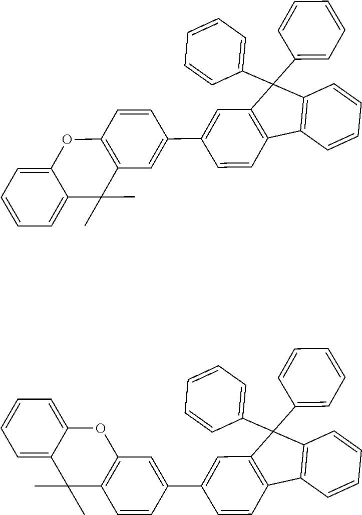 [SMILES: CC1(C)c2ccccc2Oc2ccc(-c3ccc4c(c3)C(c3ccccc3)(c3ccccc3)c3ccccc3-4)cc21 ; CC1(C)c2ccccc2Oc2cc(-c3ccc4c(c3)C(c3ccccc3)(c3ccccc3)c3ccccc3-4)ccc21]

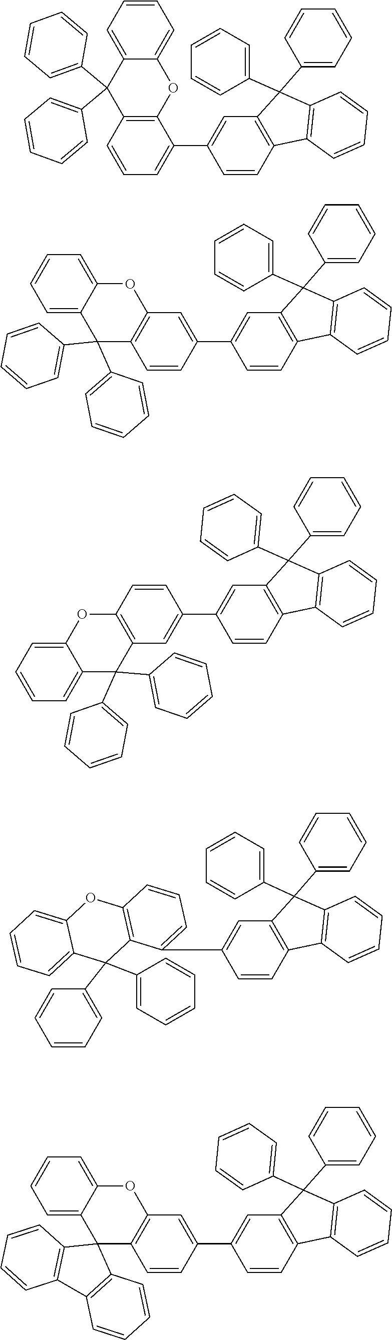 [SMILES: c1ccc(C2(c3ccccc3)c3ccccc3-c3ccc(-c4cccc5c4Oc4ccccc4C5(c4ccccc4)c4ccccc4)cc32)cc1 ; c1ccc(C2(c3ccccc3)c3ccccc3Oc3cc(-c4ccc5c(c4)C(c4ccccc4)(c4ccccc4)c4ccccc4-5)ccc32)cc1 ; c1ccc(C2(c3ccccc3)c3ccccc3Oc3ccc(-c4ccc5c(c4)C(c4ccccc4)(c4ccccc4)c4ccccc4-5)cc32)cc1 ; c1ccc(C2(c3ccccc3)c3ccccc3-c3ccc(-c4cccc5c4C(c4ccccc4)(c4ccccc4)c4ccccc4O5)cc32)cc1 ; c1ccc(C2(c3ccccc3)c3ccccc3-c3ccc(-c4ccc5c(c4)Oc4ccccc4C54c5ccccc5-c5ccccc54)cc32)cc1]

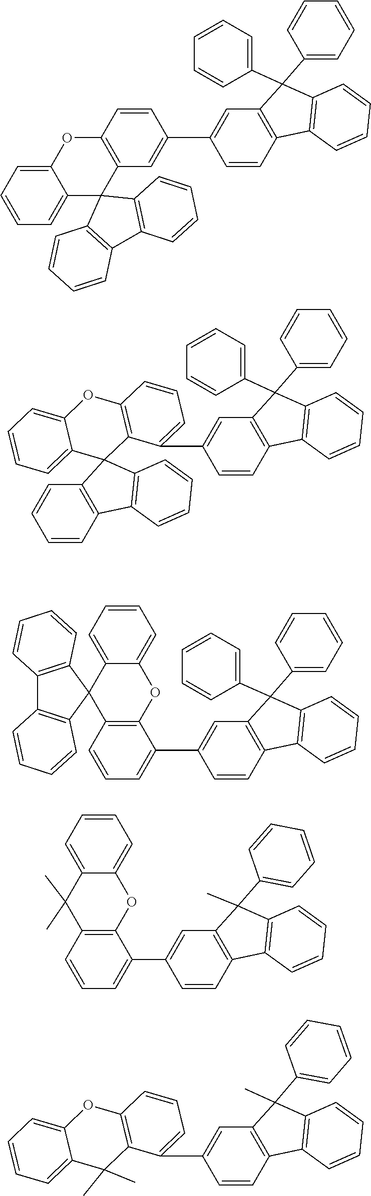

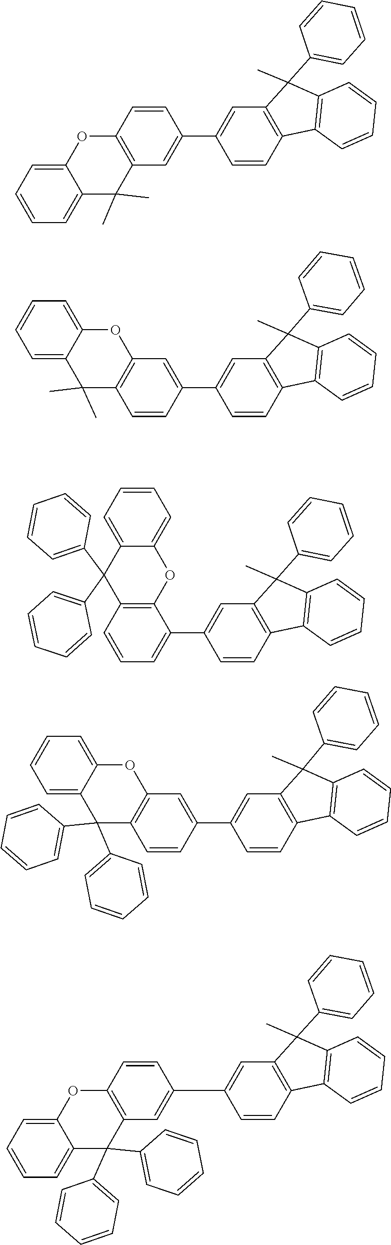

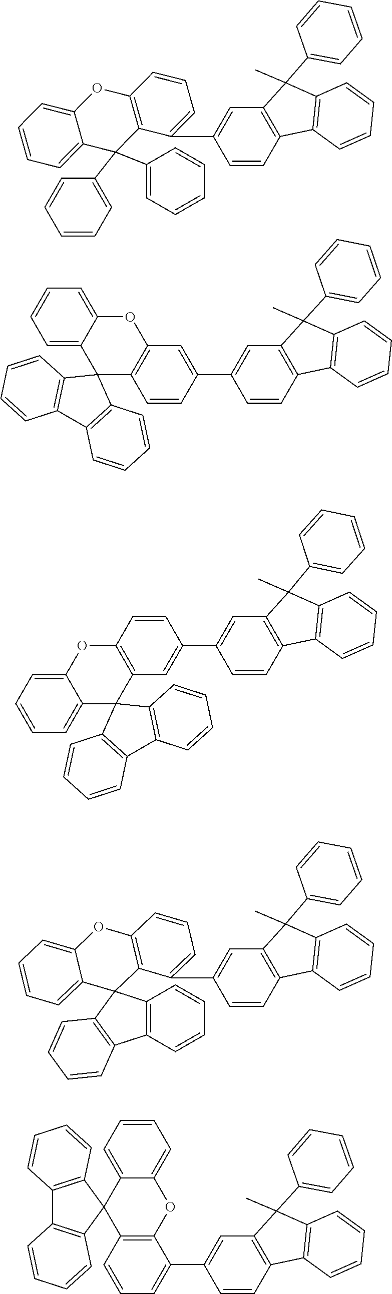

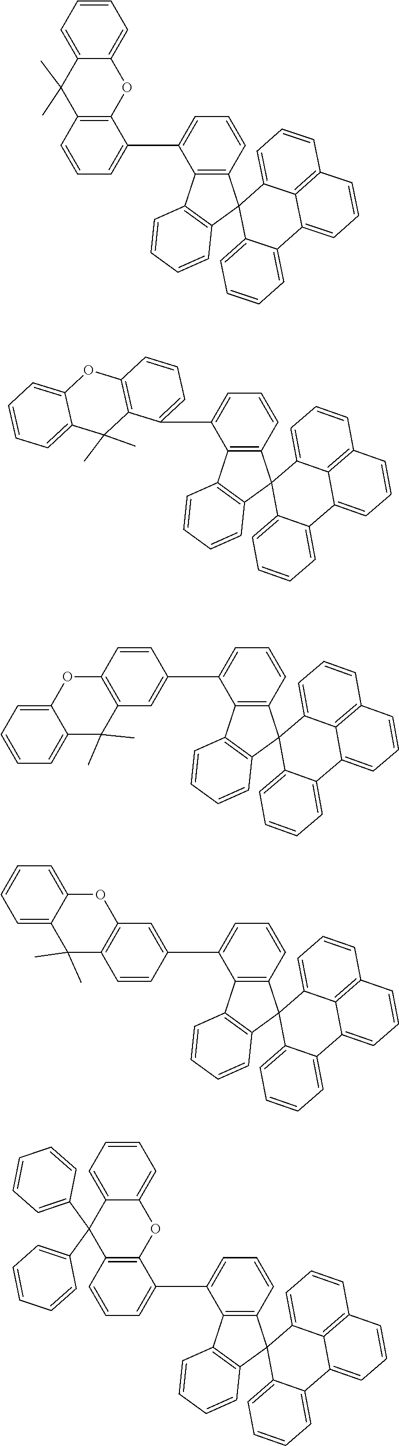 [SMILES: CC1(C)c2ccccc2Oc2c(-c3cccc4c3-c3ccccc3C43c4ccccc4-c4cccc5cccc3c45)cccc21 ; CC1(C)c2ccccc2Oc2cccc(-c3cccc4c3-c3ccccc3C43c4ccccc4-c4cccc5cccc3c45)c21 ; CC1(C)c2ccccc2Oc2ccc(-c3cccc4c3-c3ccccc3C43c4ccccc4-c4cccc5cccc3c45)cc21 ; CC1(C)c2ccccc2Oc2cc(-c3cccc4c3-c3ccccc3C43c4ccccc4-c4cccc5cccc3c45)ccc21 ; c1ccc(C2(c3ccccc3)c3ccccc3Oc3c(-c4cccc5c4-c4ccccc4C54c5ccccc5-c5cccc6cccc4c56)cccc32)cc1]

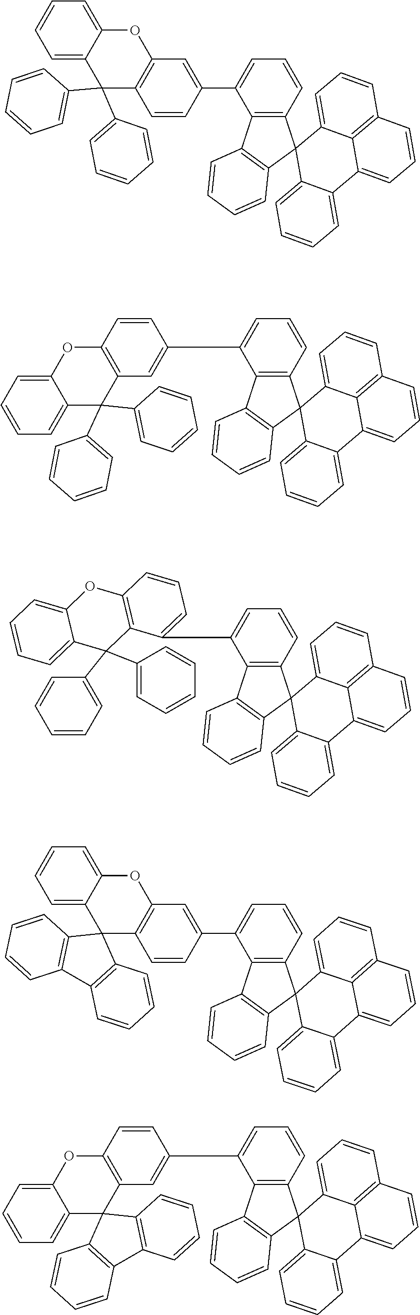

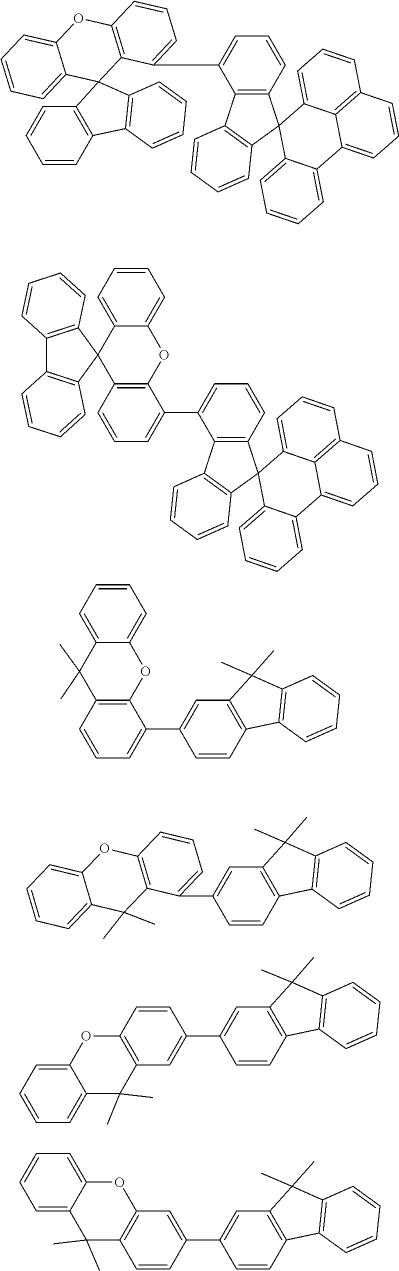

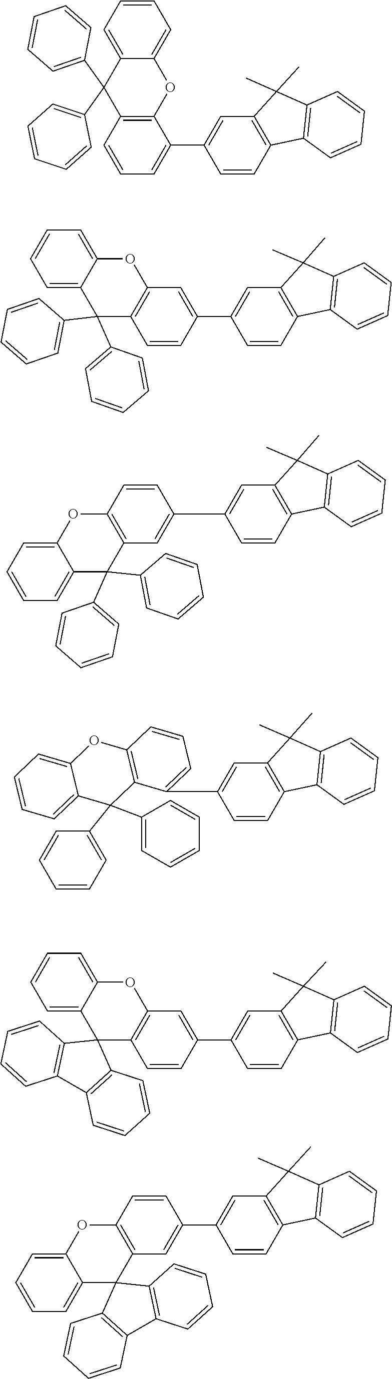 [SMILES: CC1(C)c2ccccc2-c2ccc(-c3cccc4c3Oc3ccccc3C4(c3ccccc3)c3ccccc3)cc21 ; CC1(C)c2ccccc2-c2ccc(-c3ccc4c(c3)Oc3ccccc3C4(c3ccccc3)c3ccccc3)cc21 ; CC1(C)c2ccccc2-c2ccc(-c3ccc4c(c3)C(c3ccccc3)(c3ccccc3)c3ccccc3O4)cc21 ; CC1(C)c2ccccc2-c2ccc(-c3cccc4c3C(c3ccccc3)(c3ccccc3)c3ccccc3O4)cc21 ; CC1(C)c2ccccc2-c2ccc(-c3ccc4c(c3)Oc3ccccc3C43c4ccccc4-c4ccccc43)cc21 ; CC1(C)c2ccccc2-c2ccc(-c3ccc4c(c3)C3(c5ccccc5O4)c4ccccc4-c4ccccc43)cc21]

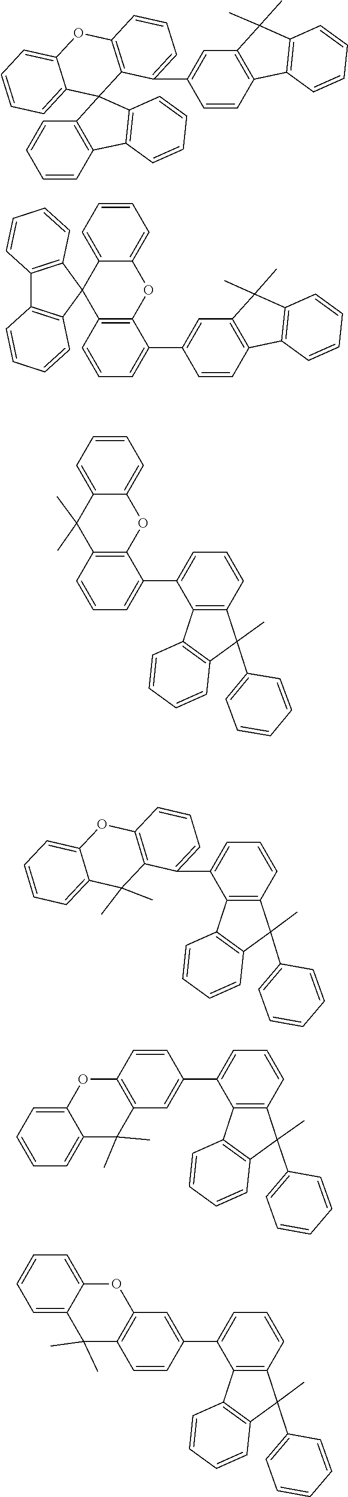

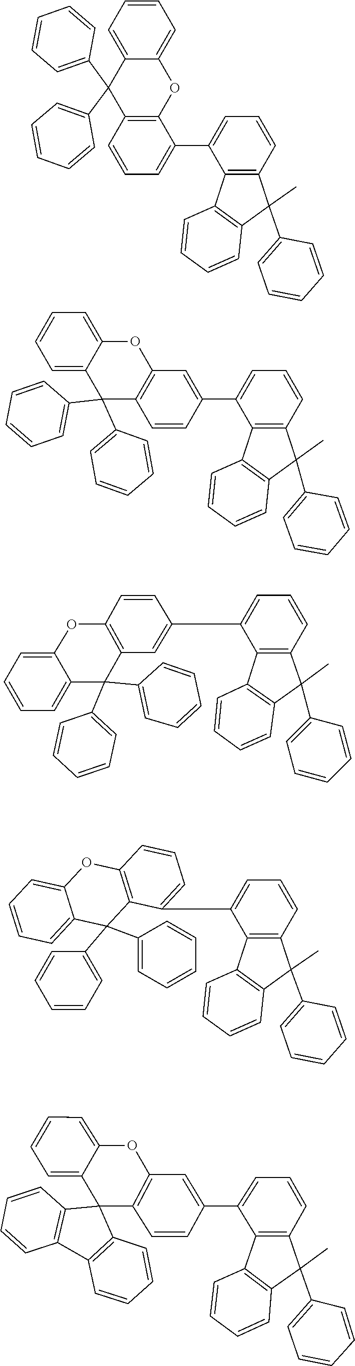

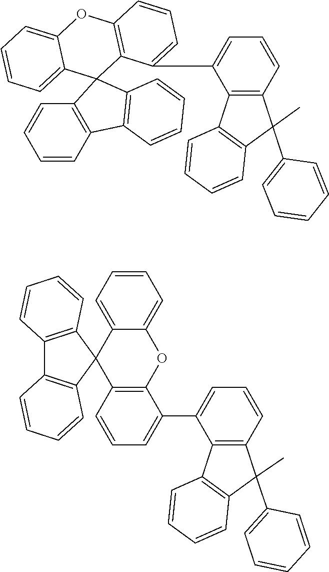 [SMILES: CC1(c2ccccc2)c2ccccc2-c2c(-c3cccc4c3C3(c5ccccc5O4)c4ccccc4-c4ccccc43)cccc21 ; CC1(c2ccccc2)c2ccccc2-c2c(-c3cccc4c3Oc3ccccc3C43c4ccccc4-c4ccccc43)cccc21]

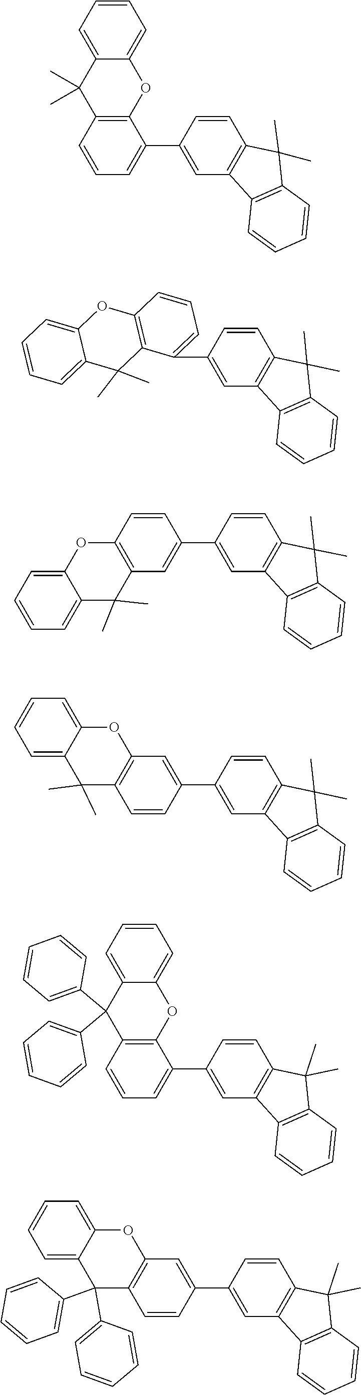

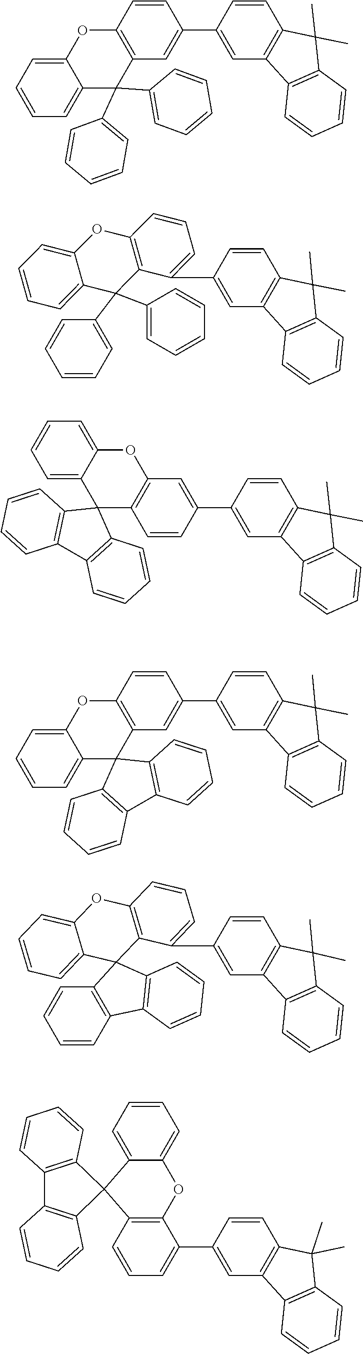

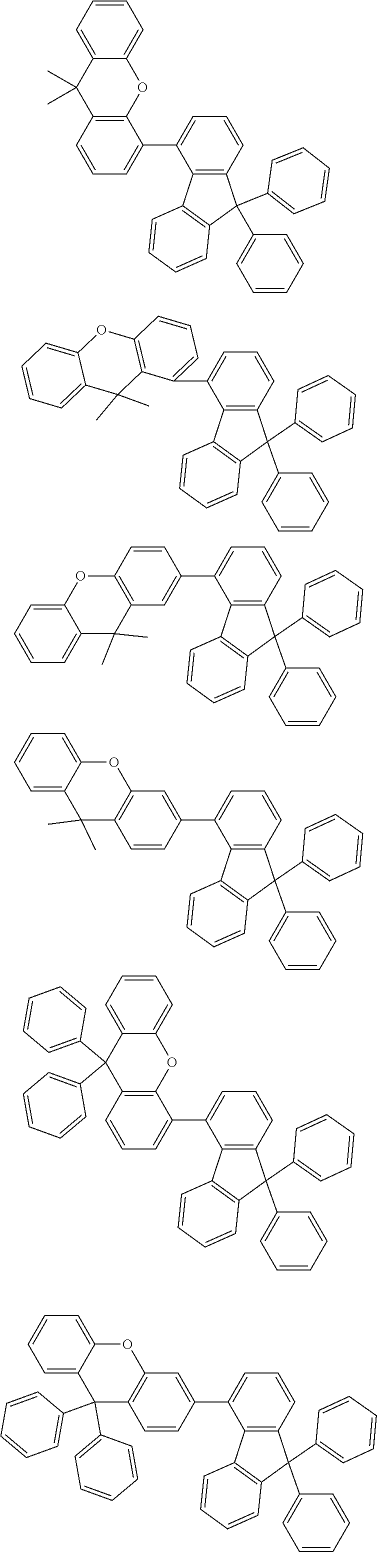

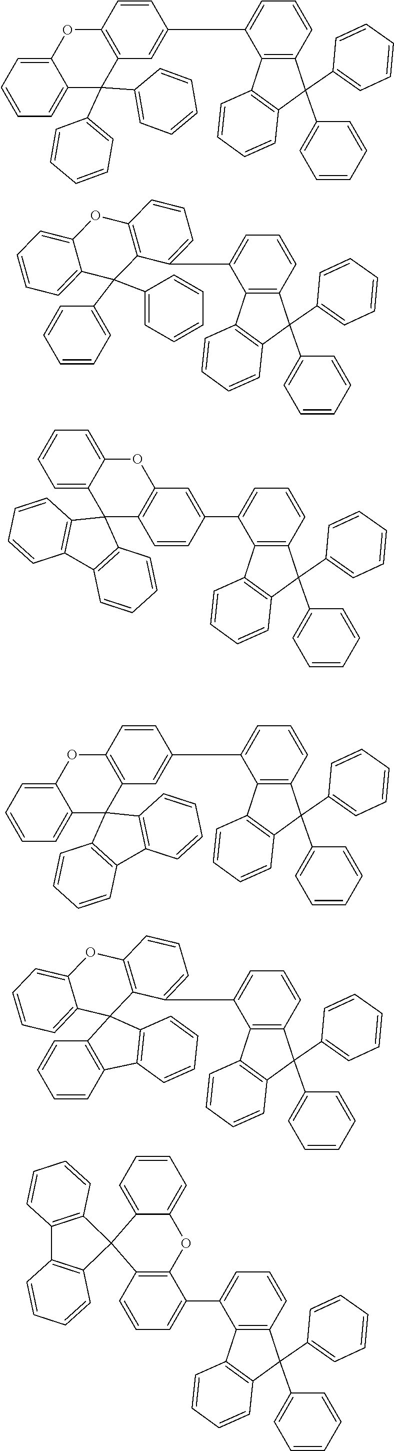

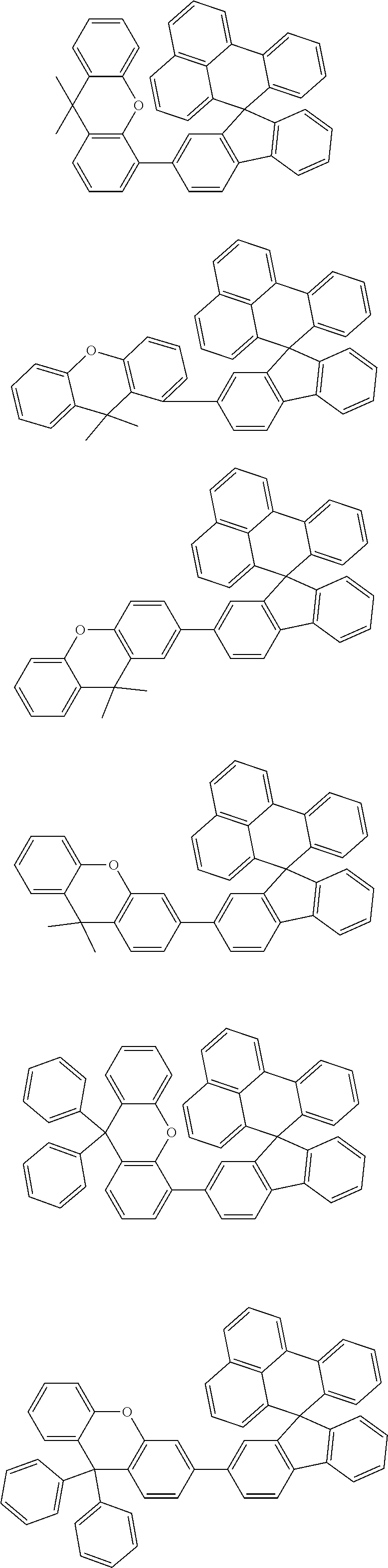

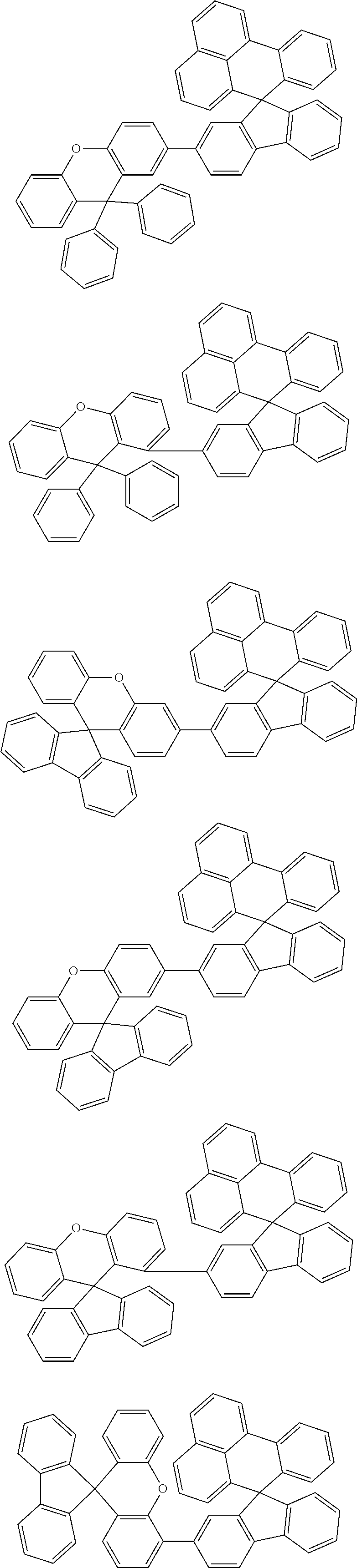

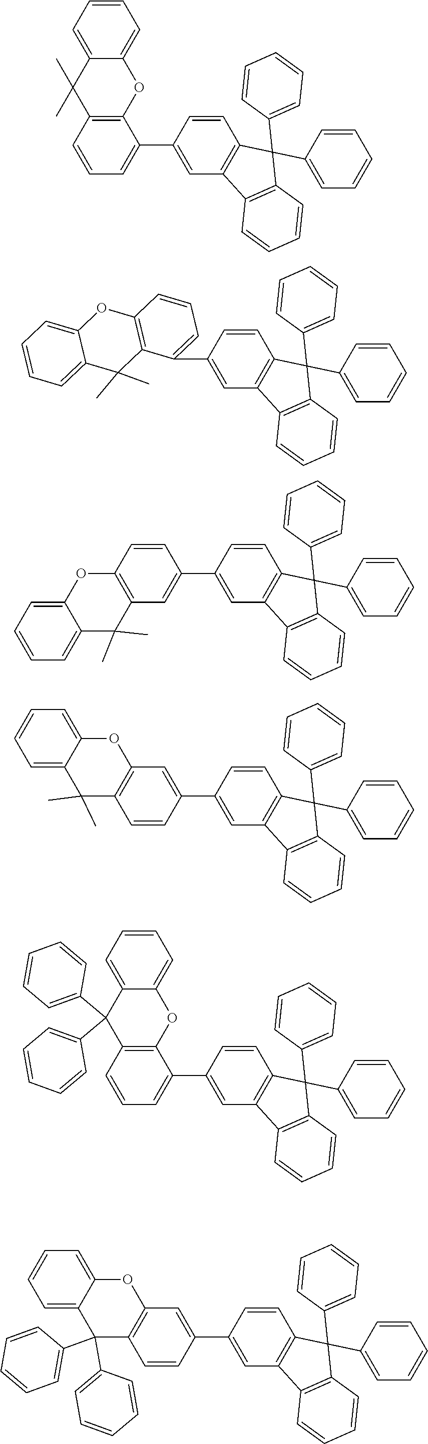

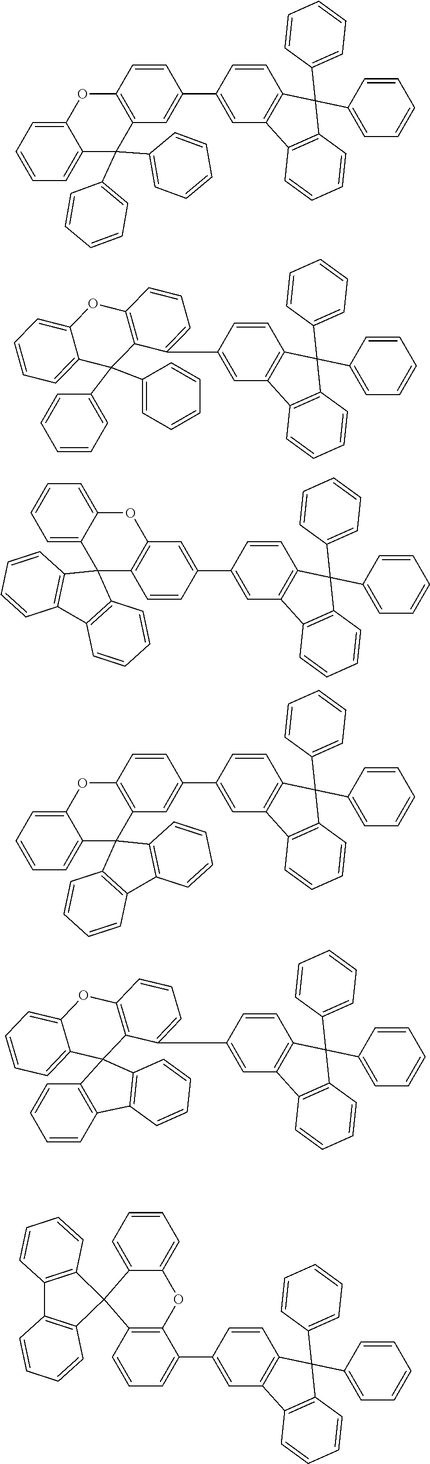

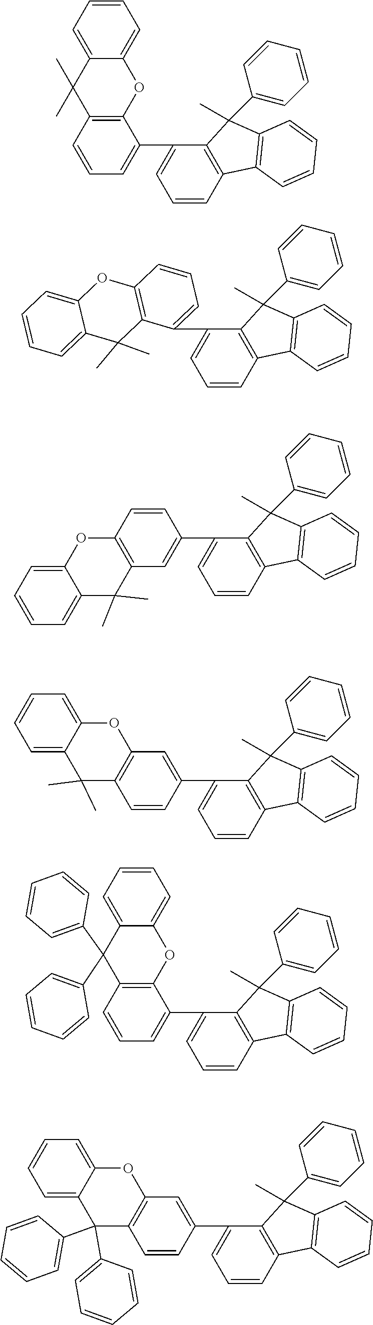

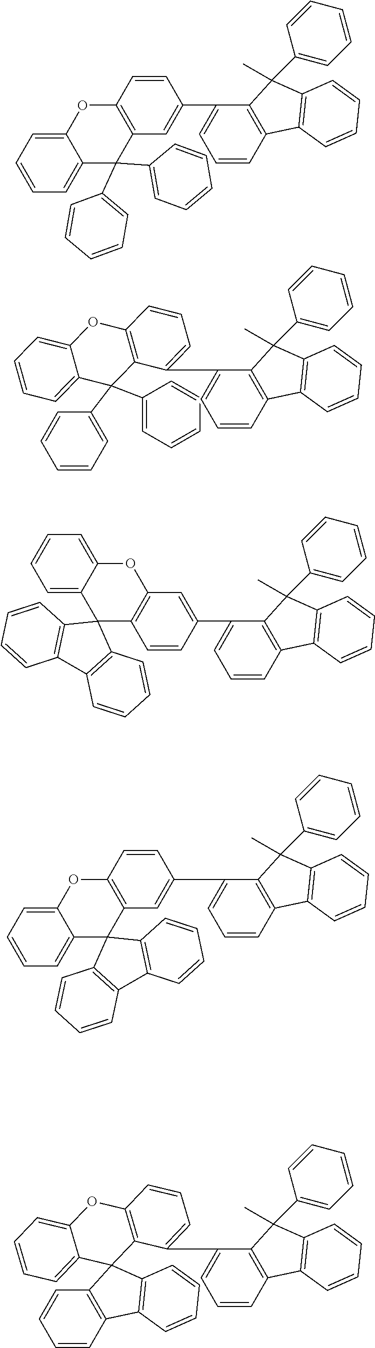 [SMILES: CC1(c2ccccc2)c2ccccc2-c2cccc(-c3ccc4c(c3)C(c3ccccc3)(c3ccccc3)c3ccccc3O4)c21 ; CC1(c2ccccc2)c2ccccc2-c2cccc(-c3cccc4c3C(c3ccccc3)(c3ccccc3)c3ccccc3O4)c21 ; CC1(c2ccccc2)c2ccccc2-c2cccc(-c3ccc4c(c3)Oc3ccccc3C43c4ccccc4-c4ccccc43)c21 ; CC1(c2ccccc2)c2ccccc2-c2cccc(-c3ccc4c(c3)C3(c5ccccc5O4)c4ccccc4-c4ccccc43)c21 ; CC1(c2ccccc2)c2ccccc2-c2cccc(-c3cccc4c3C3(c5ccccc5O4)c4ccccc4-c4ccccc43)c21]

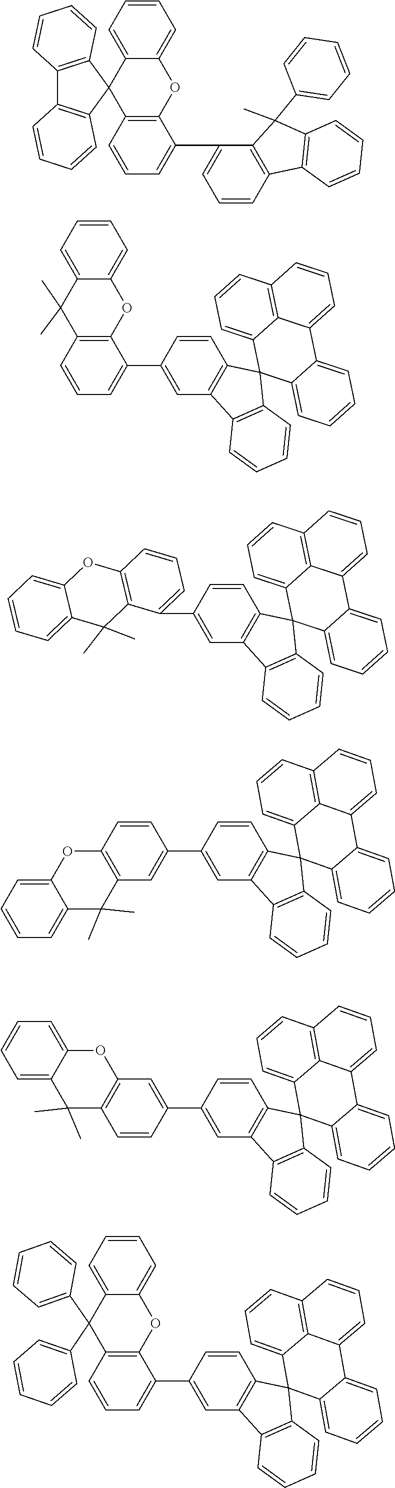

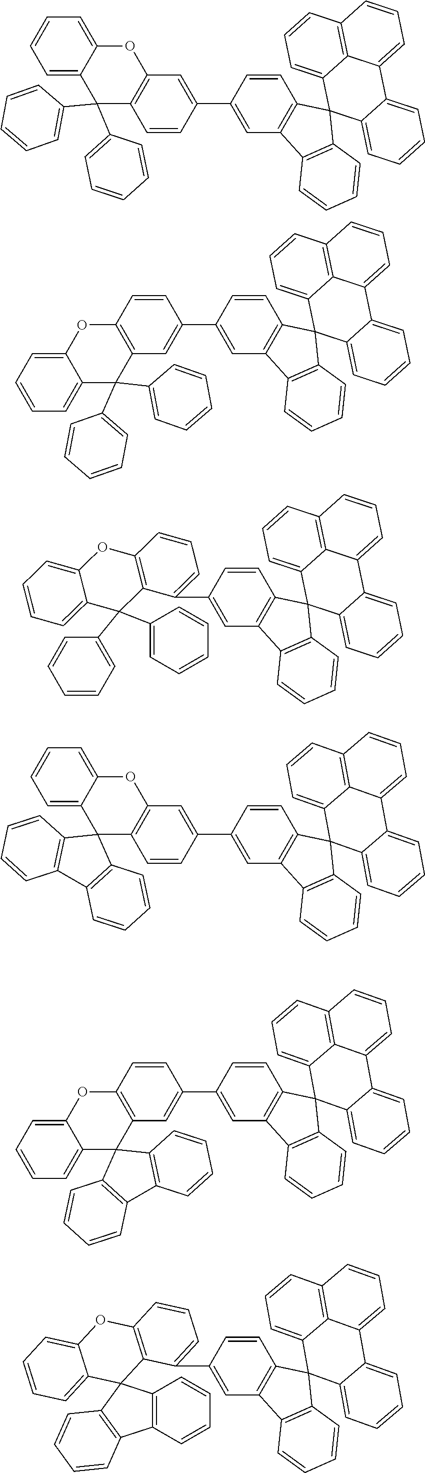

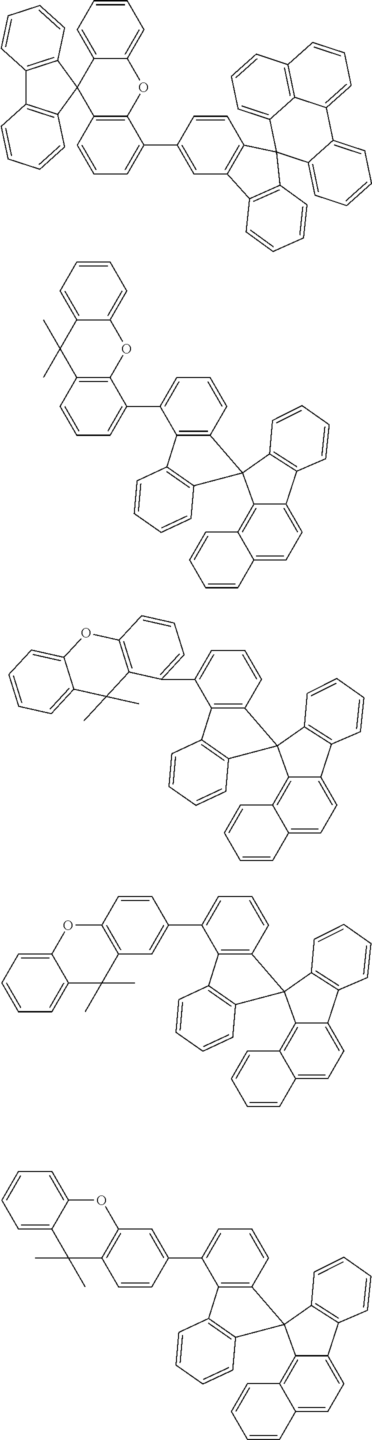

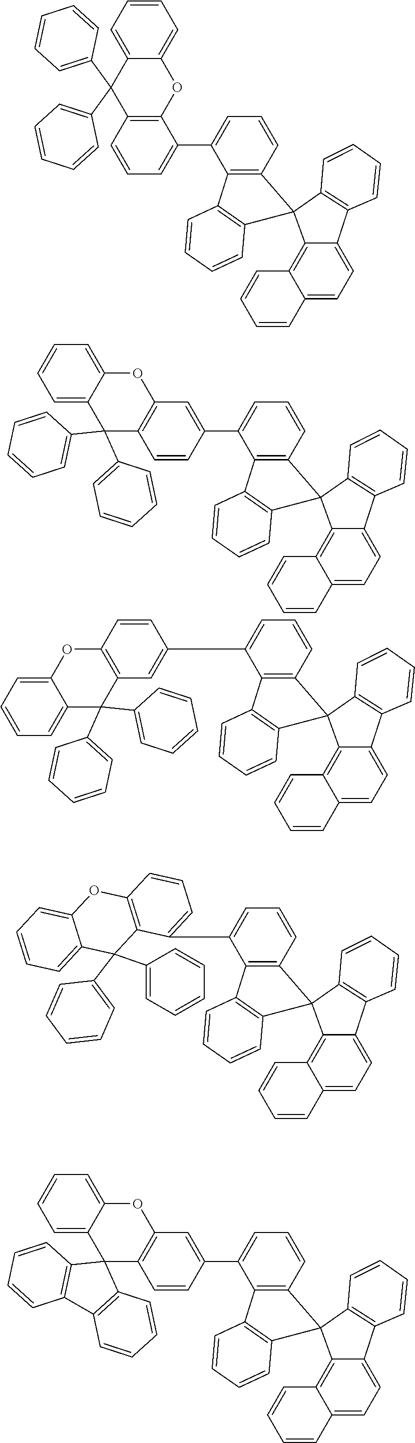

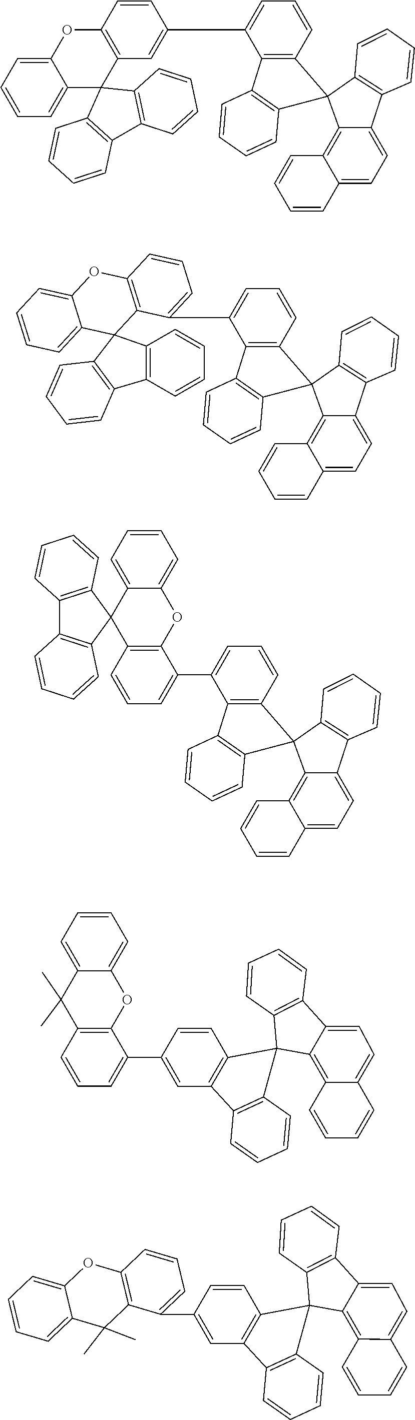 [SMILES: c1ccc2c(c1)Oc1ccc(-c3cccc4c3-c3ccccc3C43c4ccccc4-c4ccc5ccccc5c43)cc1C21c2ccccc2-c2ccccc21 ; c1ccc2c(c1)Oc1cccc(-c3cccc4c3-c3ccccc3C43c4ccccc4-c4ccc5ccccc5c43)c1C21c2ccccc2-c2ccccc21 ; c1ccc2c(c1)Oc1c(-c3cccc4c3-c3ccccc3C43c4ccccc4-c4ccc5ccccc5c43)cccc1C21c2ccccc2-c2ccccc21 ; CC1(C)c2ccccc2Oc2c(-c3ccc4c(c3)-c3ccccc3C43c4ccccc4-c4ccc5ccccc5c43)cccc21 ; CC1(C)c2ccccc2Oc2cccc(-c3ccc4c(c3)-c3ccccc3C43c4ccccc4-c4ccc5ccccc5c43)c21]

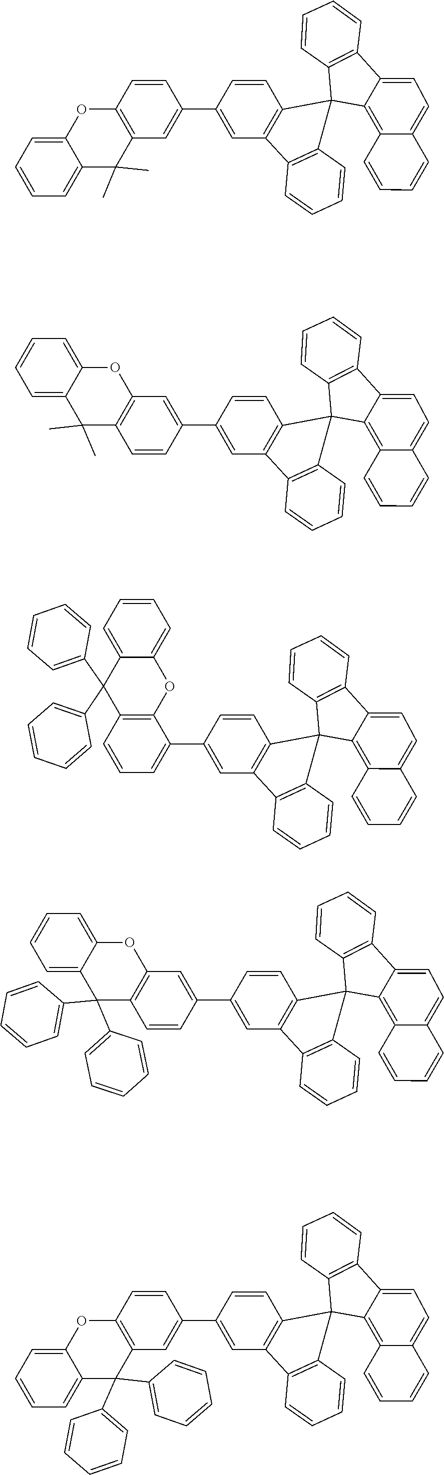 [SMILES: CC1(C)c2ccccc2Oc2ccc(-c3ccc4c(c3)-c3ccccc3C43c4ccccc4-c4ccc5ccccc5c43)cc21 ; CC1(C)c2ccccc2Oc2cc(-c3ccc4c(c3)-c3ccccc3C43c4ccccc4-c4ccc5ccccc5c43)ccc21 ; c1ccc(C2(c3ccccc3)c3ccccc3Oc3c(-c4ccc5c(c4)-c4ccccc4C54c5ccccc5-c5ccc6ccccc6c54)cccc32)cc1 ; c1ccc(C2(c3ccccc3)c3ccccc3Oc3cc(-c4ccc5c(c4)-c4ccccc4C54c5ccccc5-c5ccc6ccccc6c54)ccc32)cc1 ; c1ccc(C2(c3ccccc3)c3ccccc3Oc3ccc(-c4ccc5c(c4)-c4ccccc4C54c5ccccc5-c5ccc6ccccc6c54)cc32)cc1]

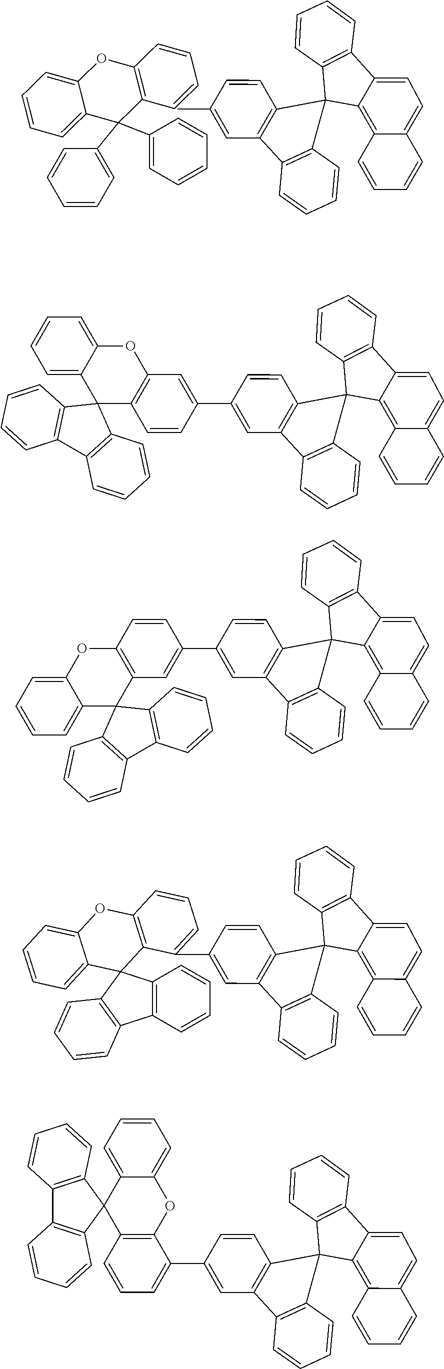

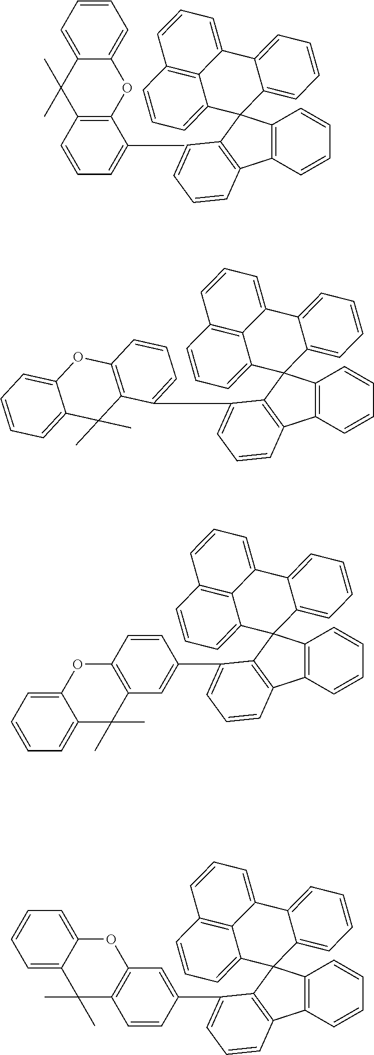

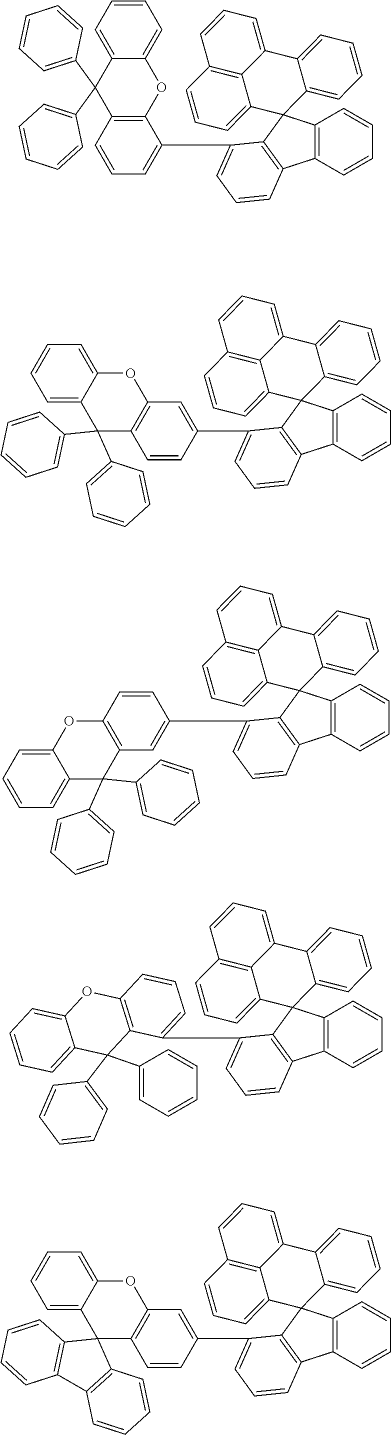 [SMILES: c1ccc(C2(c3ccccc3)c3ccccc3Oc3c(-c4cccc5c4C4(c6ccccc6-5)c5ccccc5-c5cccc6cccc4c56)cccc32)cc1 ; c1ccc(C2(c3ccccc3)c3ccccc3Oc3cc(-c4cccc5c4C4(c6ccccc6-5)c5ccccc5-c5cccc6cccc4c56)ccc32)cc1 ; c1ccc(C2(c3ccccc3)c3ccccc3Oc3ccc(-c4cccc5c4C4(c6ccccc6-5)c5ccccc5-c5cccc6cccc4c56)cc32)cc1 ; c1ccc(C2(c3ccccc3)c3ccccc3Oc3cccc(-c4cccc5c4C4(c6ccccc6-5)c5ccccc5-c5cccc6cccc4c56)c32)cc1 ; c1ccc2c(c1)Oc1cc(-c3cccc4c3C3(c5ccccc5-4)c4ccccc4-c4cccc5cccc3c45)ccc1C21c2ccccc2-c2ccccc21]

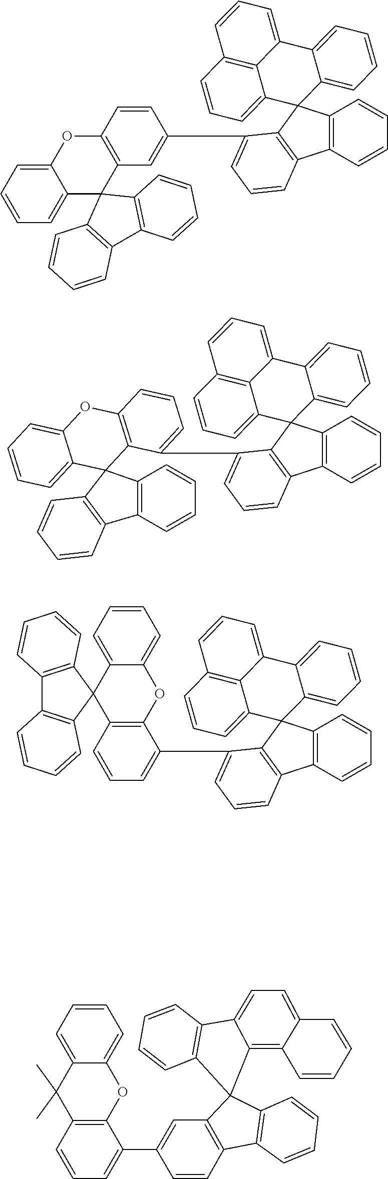 [SMILES: c1ccc2c(c1)Oc1ccc(-c3cccc4c3C3(c5ccccc5-4)c4ccccc4-c4cccc5cccc3c45)cc1C21c2ccccc2-c2ccccc21 ; c1ccc2c(c1)Oc1cccc(-c3cccc4c3C3(c5ccccc5-4)c4ccccc4-c4cccc5cccc3c45)c1C21c2ccccc2-c2ccccc21 ; c1ccc2c(c1)Oc1c(-c3cccc4c3C3(c5ccccc5-4)c4ccccc4-c4cccc5cccc3c45)cccc1C21c2ccccc2-c2ccccc21 ; CC1(C)c2ccccc2Oc2c(-c3ccc4c(c3)C3(c5ccccc5-4)c4ccccc4-c4ccc5ccccc5c43)cccc21]

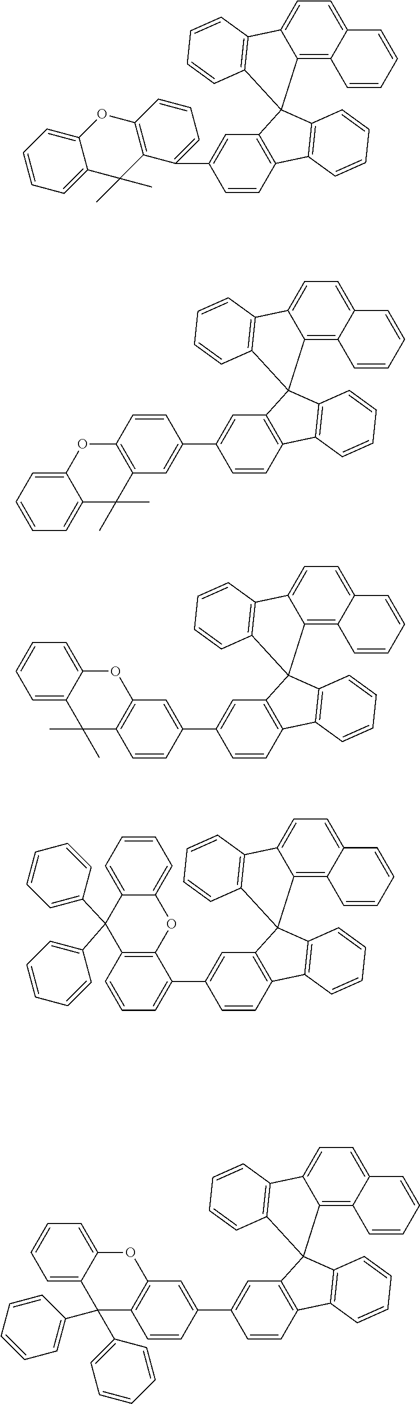 [SMILES: CC1(C)c2ccccc2Oc2cccc(-c3ccc4c(c3)C3(c5ccccc5-4)c4ccccc4-c4ccc5ccccc5c43)c21 ; CC1(C)c2ccccc2Oc2ccc(-c3ccc4c(c3)C3(c5ccccc5-4)c4ccccc4-c4ccc5ccccc5c43)cc21 ; CC1(C)c2ccccc2Oc2cc(-c3ccc4c(c3)C3(c5ccccc5-4)c4ccccc4-c4ccc5ccccc5c43)ccc21 ; c1ccc(C2(c3ccccc3)c3ccccc3Oc3c(-c4ccc5c(c4)C4(c6ccccc6-5)c5ccccc5-c5ccc6ccccc6c54)cccc32)cc1 ; c1ccc(C2(c3ccccc3)c3ccccc3Oc3cc(-c4ccc5c(c4)C4(c6ccccc6-5)c5ccccc5-c5ccc6ccccc6c54)ccc32)cc1]

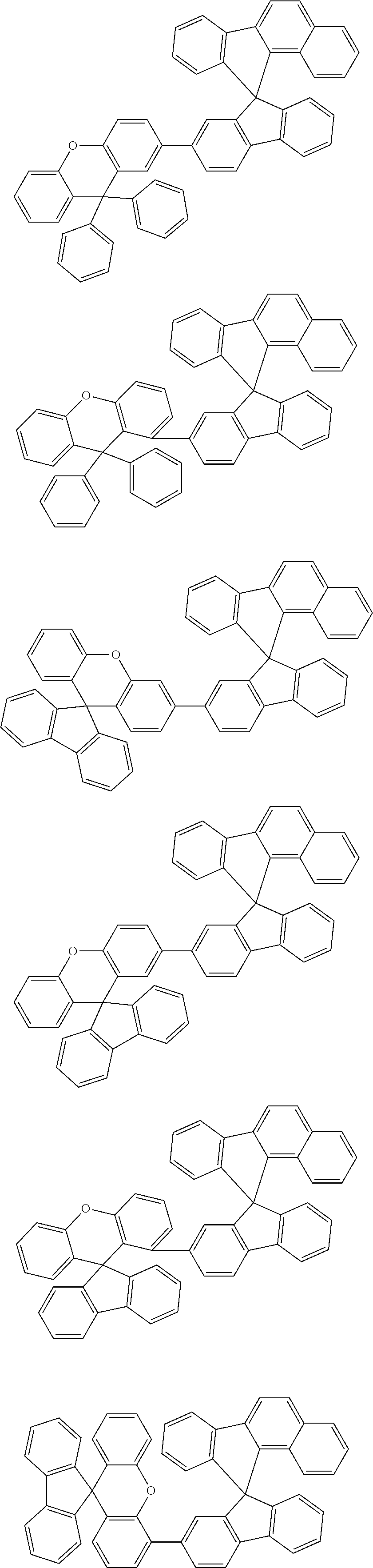

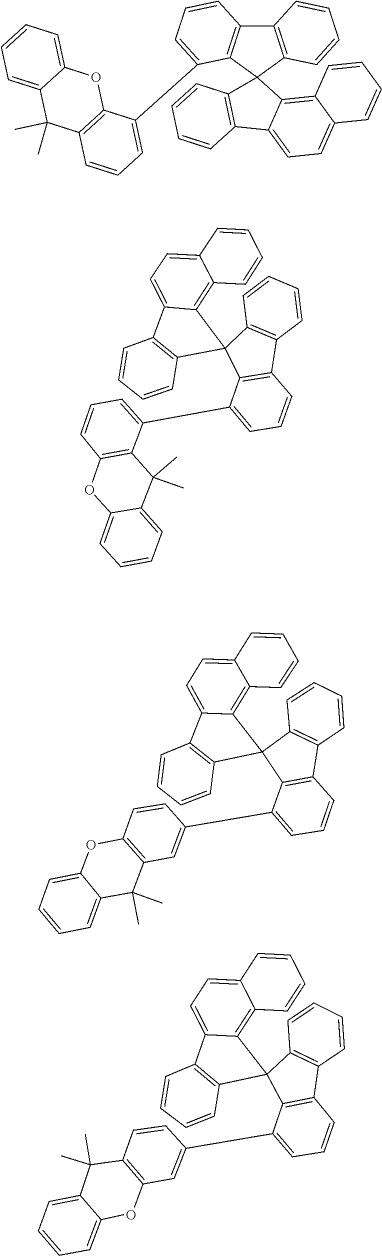 [SMILES: CC1(C)c2ccccc2Oc2c(-c3cccc4c3C3(c5ccccc5-4)c4ccccc4-c4ccc5ccccc5c43)cccc21 ; CC1(C)c2ccccc2Oc2cccc(-c3cccc4c3C3(c5ccccc5-4)c4ccccc4-c4ccc5ccccc5c43)c21 ; CC1(C)c2ccccc2Oc2ccc(-c3cccc4c3C3(c5ccccc5-4)c4ccccc4-c4ccc5ccccc5c43)cc21 ; CC1(C)c2ccccc2Oc2cc(-c3cccc4c3C3(c5ccccc5-4)c4ccccc4-c4ccc5ccccc5c43)ccc21]

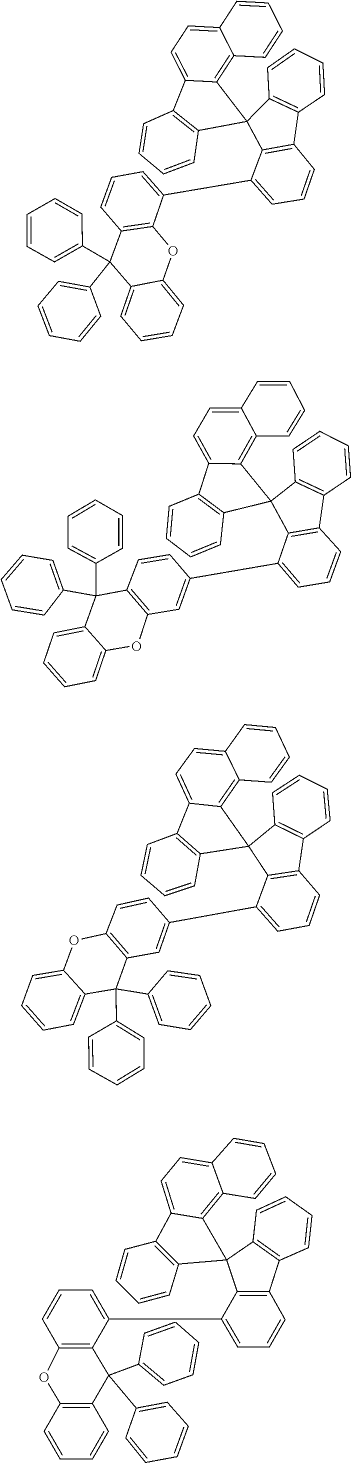 [SMILES: c1ccc(C2(c3ccccc3)c3ccccc3Oc3c(-c4cccc5c4C4(c6ccccc6-5)c5ccccc5-c5ccc6ccccc6c54)cccc32)cc1 ; c1ccc(C2(c3ccccc3)c3ccccc3Oc3cc(-c4cccc5c4C4(c6ccccc6-5)c5ccccc5-c5ccc6ccccc6c54)ccc32)cc1 ; c1ccc(C2(c3ccccc3)c3ccccc3Oc3ccc(-c4cccc5c4C4(c6ccccc6-5)c5ccccc5-c5ccc6ccccc6c54)cc32)cc1 ; c1ccc(C2(c3ccccc3)c3ccccc3Oc3cccc(-c4cccc5c4C4(c6ccccc6-5)c5ccccc5-c5ccc6ccccc6c54)c32)cc1]

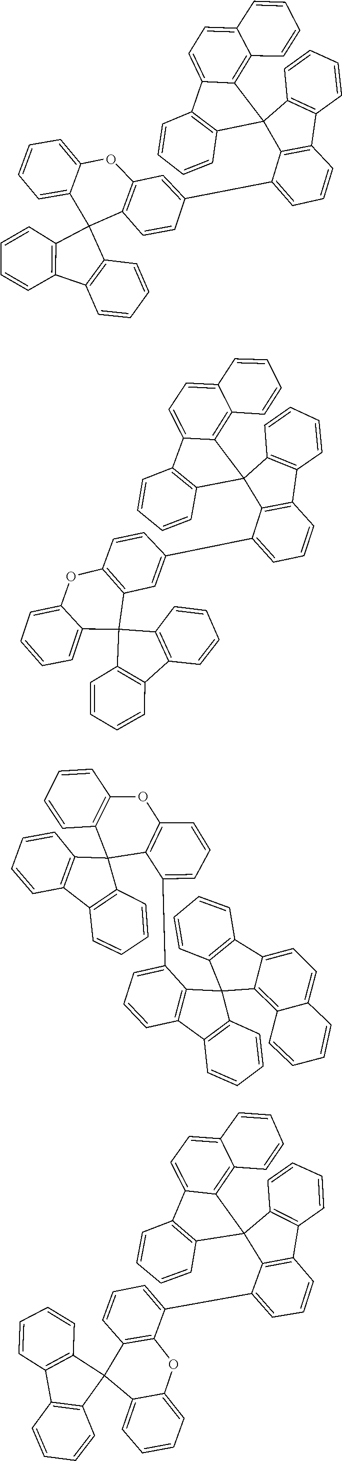

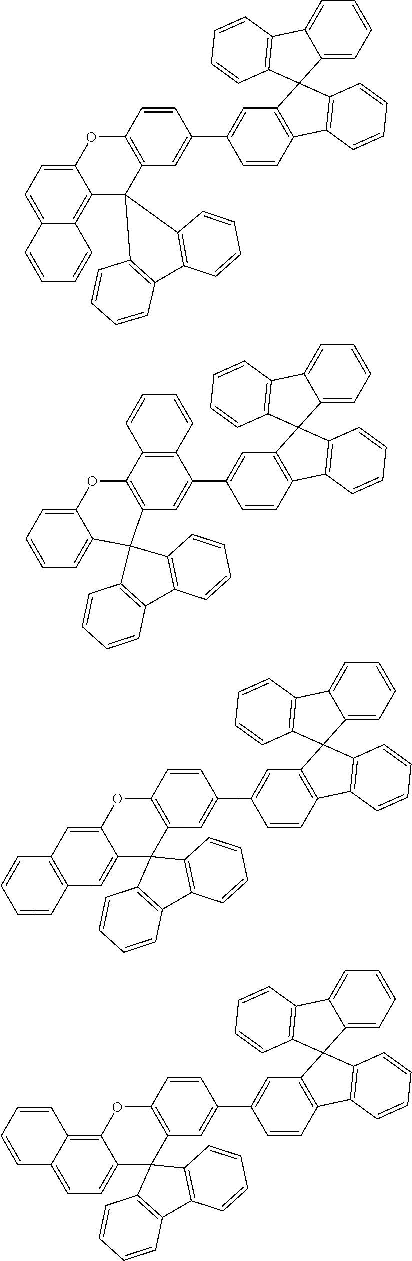 [SMILES: c1ccc2c(c1)-c1ccccc1C21c2ccccc2-c2ccc(-c3ccc4c(c3)C3(c5ccccc5-c5ccccc53)c3c(ccc5ccccc35)O4)cc21 ; c1ccc2c(c1)Oc1c(cc(-c3ccc4c(c3)C3(c5ccccc5-c5ccccc53)c3ccccc3-4)c3ccccc13)C21c2ccccc2-c2ccccc21 ; c1ccc2c(c1)-c1ccccc1C21c2cc(-c3ccc4c(c3)C3(c5ccccc5-c5ccccc53)c3ccccc3-4)ccc2Oc2cc3ccccc3cc21 ; c1ccc2c(c1)-c1ccccc1C21c2ccccc2-c2ccc(-c3ccc4c(c3)C3(c5ccccc5-c5ccccc53)c3ccc5ccccc5c3O4)cc21]

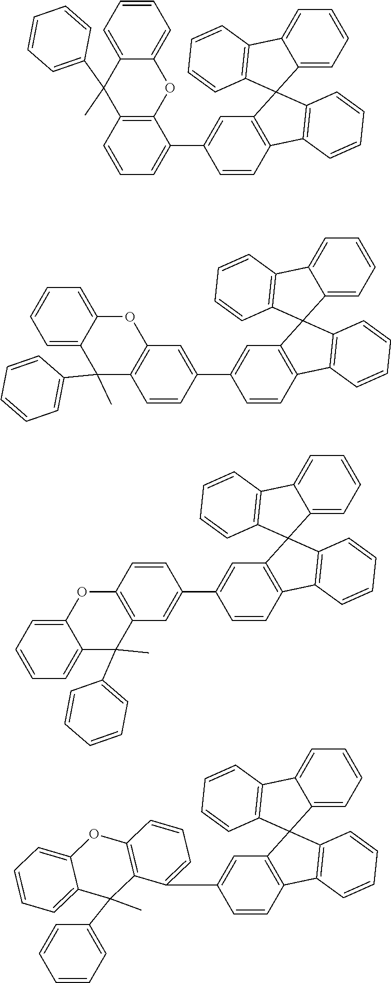 [SMILES: CC1(c2ccccc2)c2ccccc2Oc2c(-c3ccc4c(c3)C3(c5ccccc5-c5ccccc53)c3ccccc3-4)cccc21 ; CC1(c2ccccc2)c2ccccc2Oc2cc(-c3ccc4c(c3)C3(c5ccccc5-c5ccccc53)c3ccccc3-4)ccc21 ; CC1(c2ccccc2)c2ccccc2Oc2ccc(-c3ccc4c(c3)C3(c5ccccc5-c5ccccc53)c3ccccc3-4)cc21 ; CC1(c2ccccc2)c2ccccc2Oc2cccc(-c3ccc4c(c3)C3(c5ccccc5-c5ccccc53)c3ccccc3-4)c21]

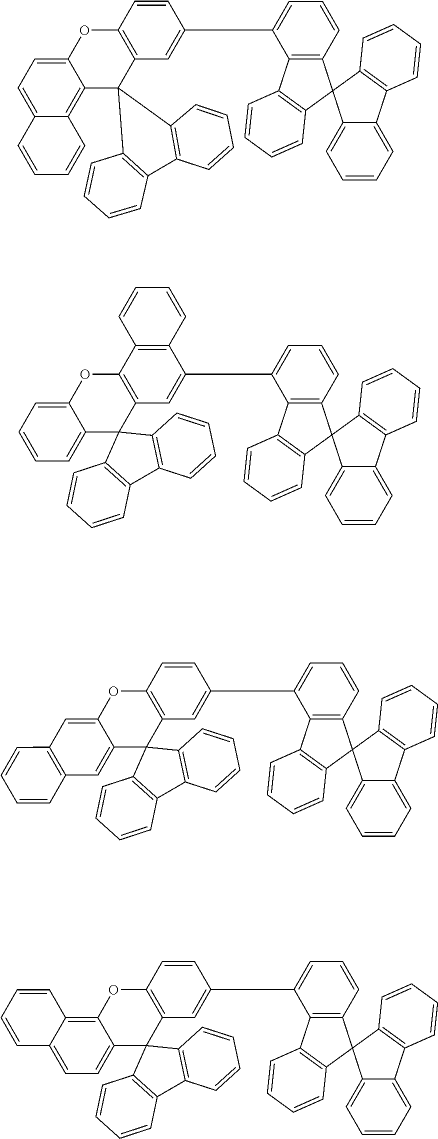 [SMILES: c1ccc2c(c1)-c1ccccc1C21c2ccccc2-c2c(-c3ccc4c(c3)C3(c5ccccc5-c5ccccc53)c3c(ccc5ccccc35)O4)cccc21 ; c1ccc2c(c1)Oc1c(cc(-c3cccc4c3-c3ccccc3C43c4ccccc4-c4ccccc43)c3ccccc13)C21c2ccccc2-c2ccccc21 ; c1ccc2c(c1)-c1ccccc1C21c2cc(-c3cccc4c3-c3ccccc3C43c4ccccc4-c4ccccc43)ccc2Oc2cc3ccccc3cc21 ; c1ccc2c(c1)-c1ccccc1C21c2ccccc2-c2c(-c3ccc4c(c3)C3(c5ccccc5-c5ccccc53)c3ccc5ccccc5c3O4)cccc21]

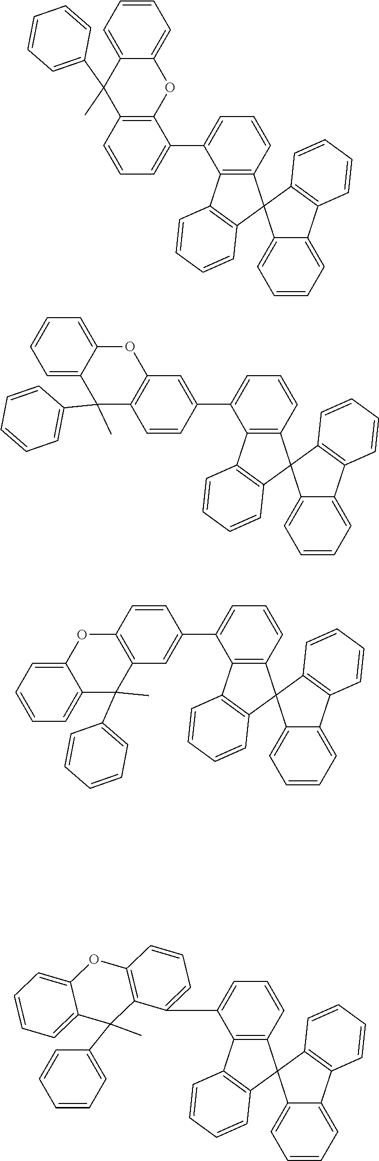 [SMILES: CC1(c2ccccc2)c2ccccc2Oc2c(-c3cccc4c3-c3ccccc3C43c4ccccc4-c4ccccc43)cccc21 ; CC1(c2ccccc2)c2ccccc2Oc2cc(-c3cccc4c3-c3ccccc3C43c4ccccc4-c4ccccc43)ccc21 ; CC1(c2ccccc2)c2ccccc2Oc2ccc(-c3cccc4c3-c3ccccc3C43c4ccccc4-c4ccccc43)cc21 ; CC1(c2ccccc2)c2ccccc2Oc2cccc(-c3cccc4c3-c3ccccc3C43c4ccccc4-c4ccccc43)c21]

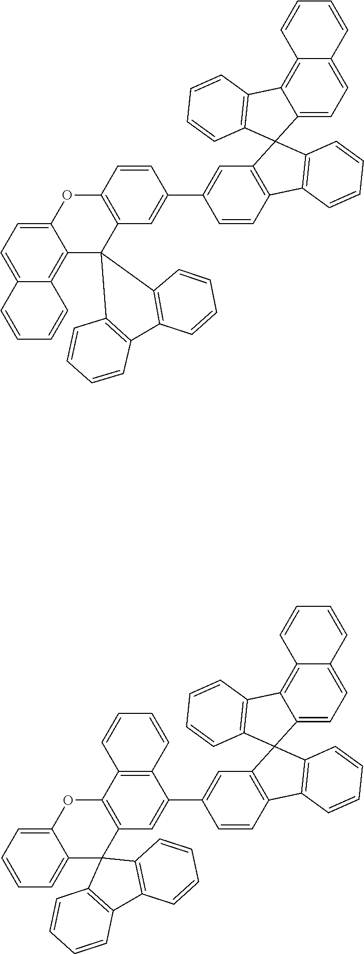 [SMILES: c1ccc2c(c1)-c1ccc(-c3ccc4c(c3)C3(c5ccccc5-c5ccccc53)c3c(ccc5ccccc35)O4)cc1C21c2ccccc2-c2c1ccc1ccccc21 ; c1ccc2c(c1)Oc1c(cc(-c3ccc4c(c3)C3(c5ccccc5-4)c4ccccc4-c4c3ccc3ccccc43)c3ccccc13)C21c2ccccc2-c2ccccc21]

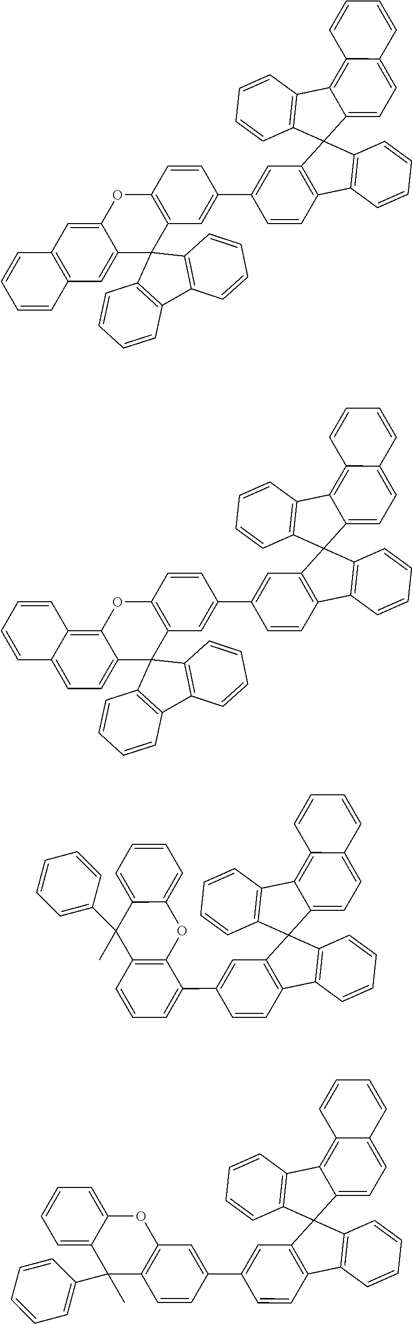 [SMILES: c1ccc2c(c1)-c1ccccc1C21c2cc(-c3ccc4c(c3)C3(c5ccccc5-4)c4ccccc4-c4c3ccc3ccccc43)ccc2Oc2cc3ccccc3cc21 ; c1ccc2c(c1)-c1ccccc1C21c2cc(-c3ccc4c(c3)C3(c5ccccc5-4)c4ccccc4-c4c3ccc3ccccc43)ccc2Oc2c1ccc1ccccc21 ; CC1(c2ccccc2)c2ccccc2Oc2c(-c3ccc4c(c3)C3(c5ccccc5-4)c4ccccc4-c4c3ccc3ccccc43)cccc21 ; CC1(c2ccccc2)c2ccccc2Oc2cc(-c3ccc4c(c3)C3(c5ccccc5-4)c4ccccc4-c4c3ccc3ccccc43)ccc21]

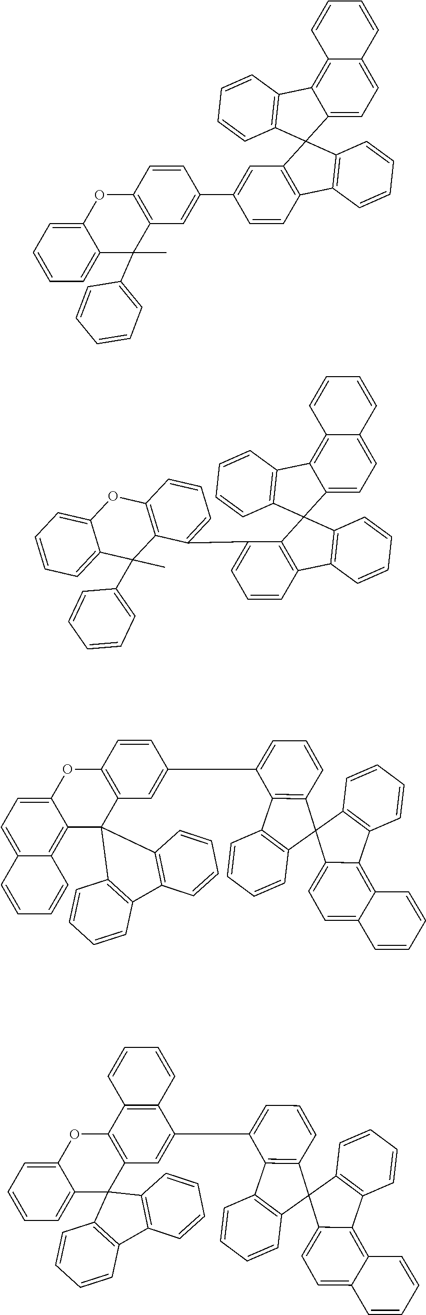 [SMILES: CC1(c2ccccc2)c2ccccc2Oc2ccc(-c3ccc4c(c3)C3(c5ccccc5-4)c4ccccc4-c4c3ccc3ccccc43)cc21 ; CC1(c2ccccc2)c2ccccc2Oc2cccc(-c3cccc4c3C3(c5ccccc5-4)c4ccccc4-c4c3ccc3ccccc43)c21 ; c1ccc2c(c1)-c1c(-c3ccc4c(c3)C3(c5ccccc5-c5ccccc53)c3c(ccc5ccccc35)O4)cccc1C21c2ccccc2-c2c1ccc1ccccc21 ; c1ccc2c(c1)Oc1c(cc(-c3cccc4c3-c3ccccc3C43c4ccccc4-c4c3ccc3ccccc43)c3ccccc13)C21c2ccccc2-c2ccccc21]

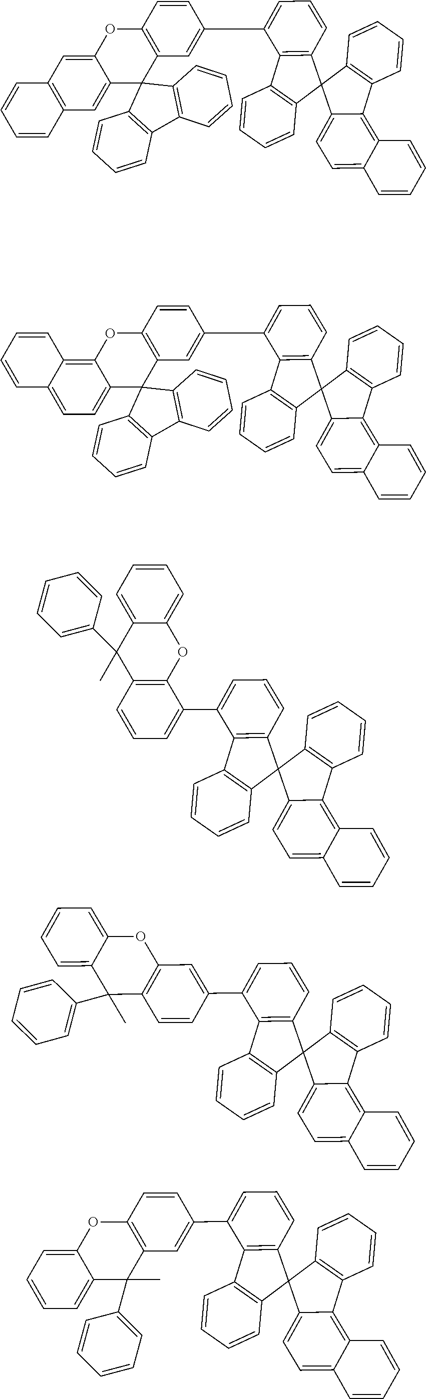 [SMILES: c1ccc2c(c1)-c1ccccc1C21c2cc(-c3cccc4c3-c3ccccc3C43c4ccccc4-c4c3ccc3ccccc43)ccc2Oc2cc3ccccc3cc21 ; c1ccc2c(c1)-c1ccccc1C21c2cc(-c3cccc4c3-c3ccccc3C43c4ccccc4-c4c3ccc3ccccc43)ccc2Oc2c1ccc1ccccc21 ; CC1(c2ccccc2)c2ccccc2Oc2c(-c3cccc4c3-c3ccccc3C43c4ccccc4-c4c3ccc3ccccc43)cccc21 ; CC1(c2ccccc2)c2ccccc2Oc2cc(-c3cccc4c3-c3ccccc3C43c4ccccc4-c4c3ccc3ccccc43)ccc21 ; CC1(c2ccccc2)c2ccccc2Oc2ccc(-c3cccc4c3-c3ccccc3C43c4ccccc4-c4c3ccc3ccccc43)cc21]

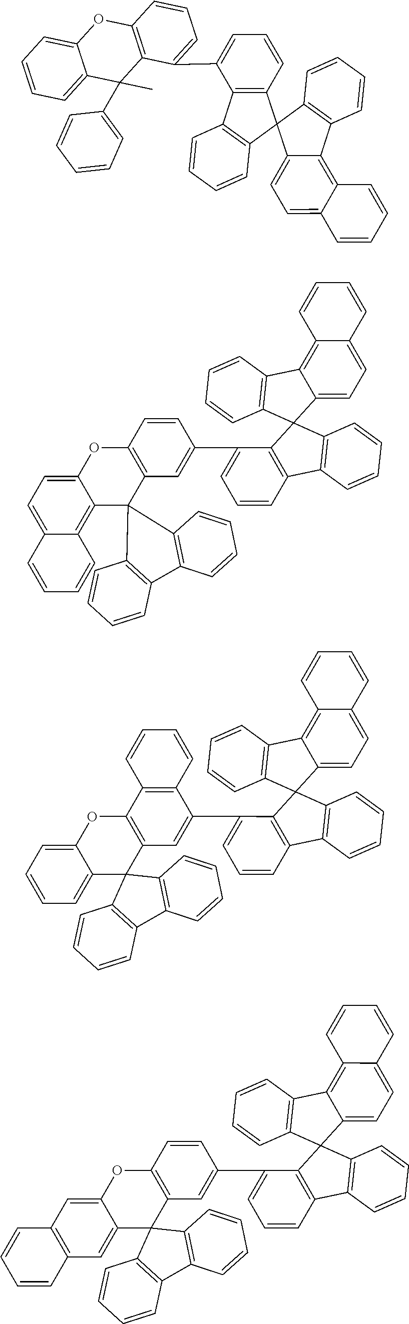

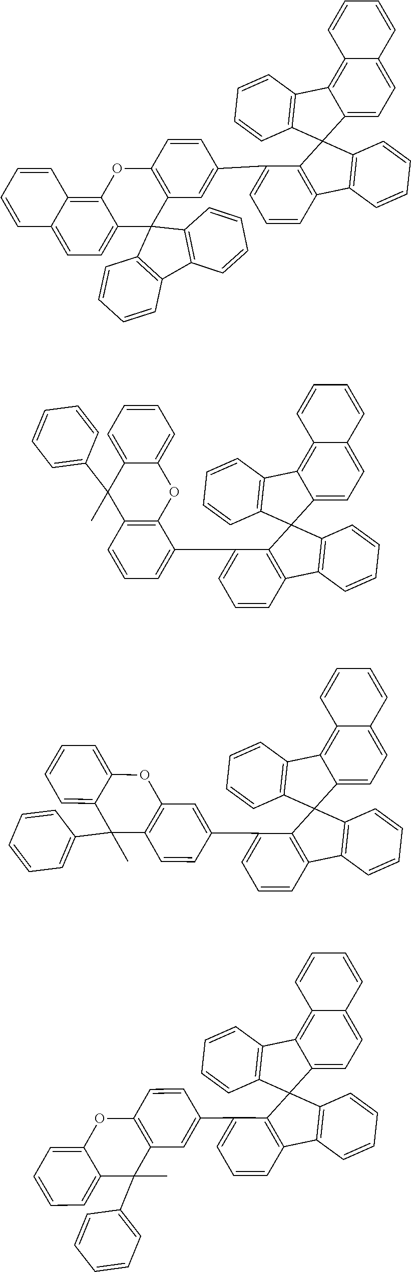 [SMILES: c1ccc2c(c1)-c1ccccc1C21c2cc(-c3cccc4c3C3(c5ccccc5-4)c4ccccc4-c4c3ccc3ccccc43)ccc2Oc2c1ccc1ccccc21 ; CC1(c2ccccc2)c2ccccc2Oc2c(-c3cccc4c3C3(c5ccccc5-4)c4ccccc4-c4c3ccc3ccccc43)cccc21 ; CC1(c2ccccc2)c2ccccc2Oc2cc(-c3cccc4c3C3(c5ccccc5-4)c4ccccc4-c4c3ccc3ccccc43)ccc21 ; CC1(c2ccccc2)c2ccccc2Oc2ccc(-c3cccc4c3C3(c5ccccc5-4)c4ccccc4-c4c3ccc3ccccc43)cc21]

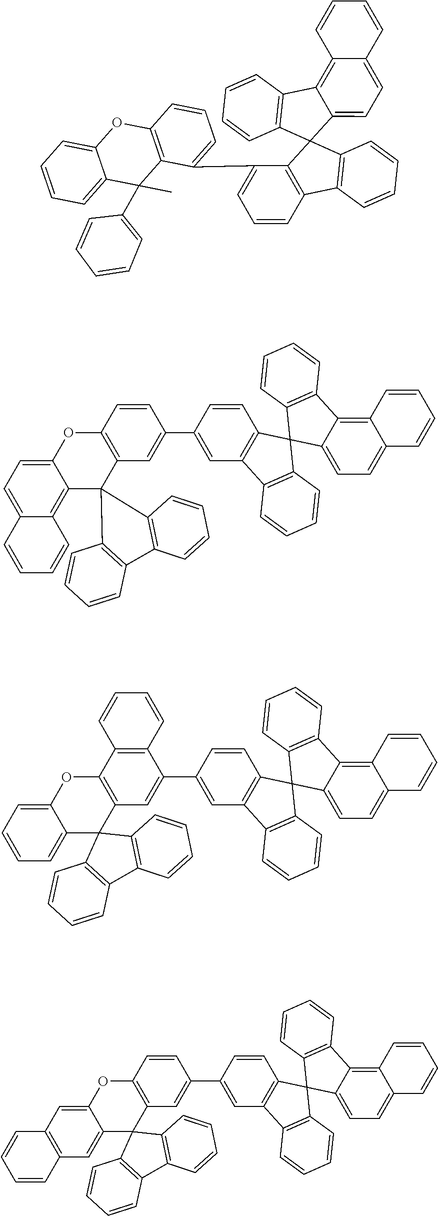 [SMILES: CC1(c2ccccc2)c2ccccc2Oc2cccc(-c3cccc4c3C3(c5ccccc5-4)c4ccccc4-c4c3ccc3ccccc43)c21 ; c1ccc2c(c1)-c1cc(-c3ccc4c(c3)C3(c5ccccc5-c5ccccc53)c3c(ccc5ccccc35)O4)ccc1C21c2ccccc2-c2c1ccc1ccccc21 ; c1ccc2c(c1)Oc1c(cc(-c3ccc4c(c3)-c3ccccc3C43c4ccccc4-c4c3ccc3ccccc43)c3ccccc13)C21c2ccccc2-c2ccccc21 ; c1ccc2c(c1)-c1ccccc1C21c2cc(-c3ccc4c(c3)-c3ccccc3C43c4ccccc4-c4c3ccc3ccccc43)ccc2Oc2cc3ccccc3cc21]

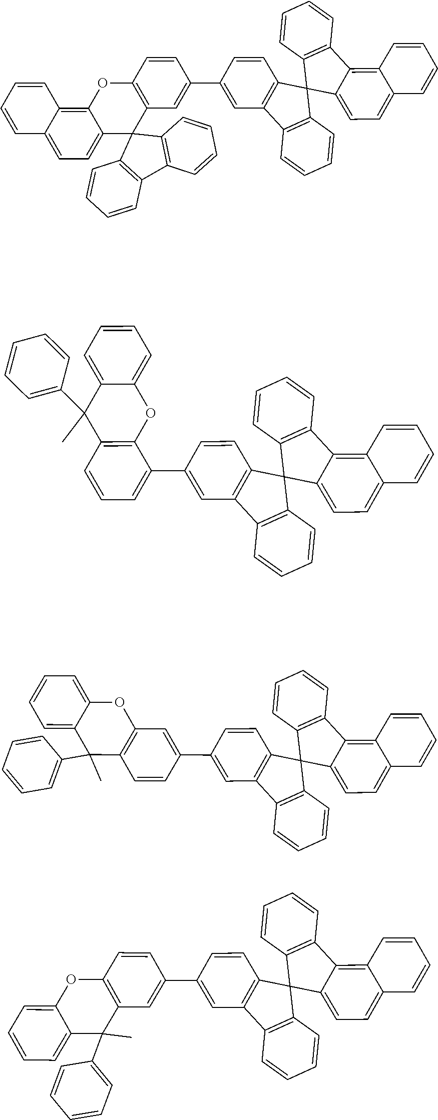 [SMILES: c1ccc2c(c1)-c1ccccc1C21c2cc(-c3ccc4c(c3)-c3ccccc3C43c4ccccc4-c4c3ccc3ccccc43)ccc2Oc2c1ccc1ccccc21 ; CC1(c2ccccc2)c2ccccc2Oc2c(-c3ccc4c(c3)-c3ccccc3C43c4ccccc4-c4c3ccc3ccccc43)cccc21 ; CC1(c2ccccc2)c2ccccc2Oc2cc(-c3ccc4c(c3)-c3ccccc3C43c4ccccc4-c4c3ccc3ccccc43)ccc21 ; CC1(c2ccccc2)c2ccccc2Oc2ccc(-c3ccc4c(c3)-c3ccccc3C43c4ccccc4-c4c3ccc3ccccc43)cc21]

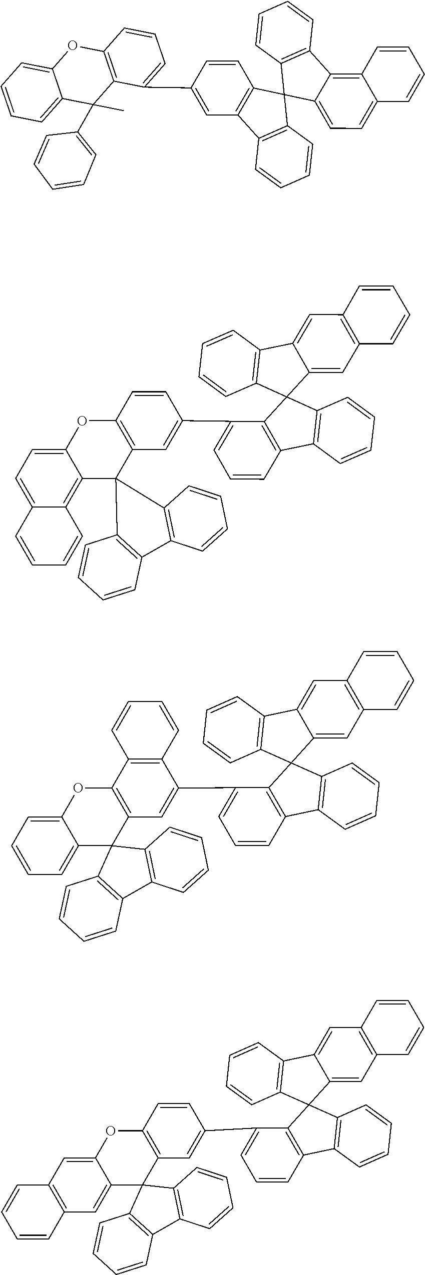 [SMILES: CC1(c2ccccc2)c2ccccc2Oc2cccc(-c3ccc4c(c3)-c3ccccc3C43c4ccccc4-c4c3ccc3ccccc43)c21 ; c1ccc2c(c1)-c1cc3ccccc3cc1C21c2ccccc2-c2cccc(-c3ccc4c(c3)C3(c5ccccc5-c5ccccc53)c3c(ccc5ccccc35)O4)c21 ; c1ccc2c(c1)Oc1c(cc(-c3cccc4c3C3(c5ccccc5-c5cc6ccccc6cc53)c3ccccc3-4)c3ccccc13)C21c2ccccc2-c2ccccc21 ; c1ccc2c(c1)-c1ccccc1C21c2cc(-c3cccc4c3C3(c5ccccc5-c5cc6ccccc6cc53)c3ccccc3-4)ccc2Oc2cc3ccccc3cc21]

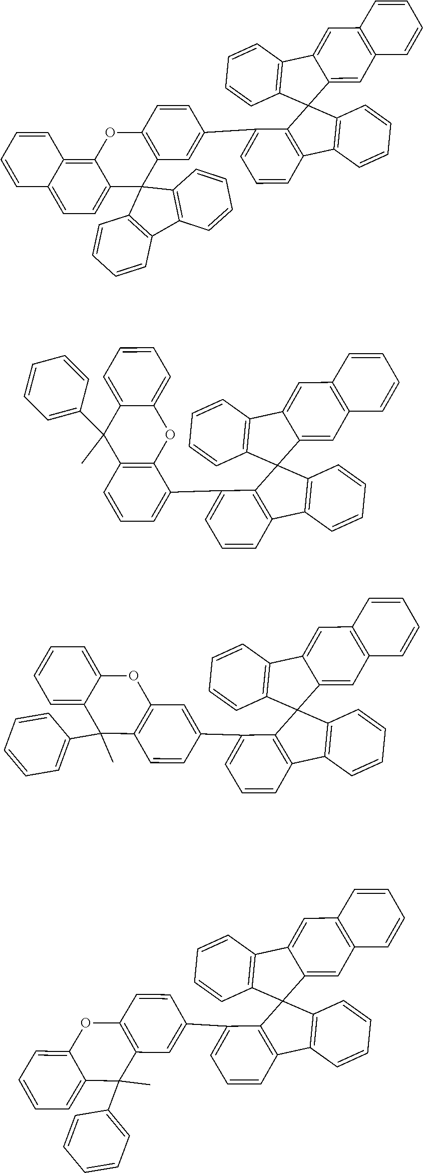 [SMILES: c1ccc2c(c1)-c1ccccc1C21c2cc(-c3cccc4c3C3(c5ccccc5-c5cc6ccccc6cc53)c3ccccc3-4)ccc2Oc2c1ccc1ccccc21 ; CC1(c2ccccc2)c2ccccc2Oc2c(-c3cccc4c3C3(c5ccccc5-c5cc6ccccc6cc53)c3ccccc3-4)cccc21 ; CC1(c2ccccc2)c2ccccc2Oc2cc(-c3cccc4c3C3(c5ccccc5-c5cc6ccccc6cc53)c3ccccc3-4)ccc21 ; CC1(c2ccccc2)c2ccccc2Oc2ccc(-c3cccc4c3C3(c5ccccc5-c5cc6ccccc6cc53)c3ccccc3-4)cc21]

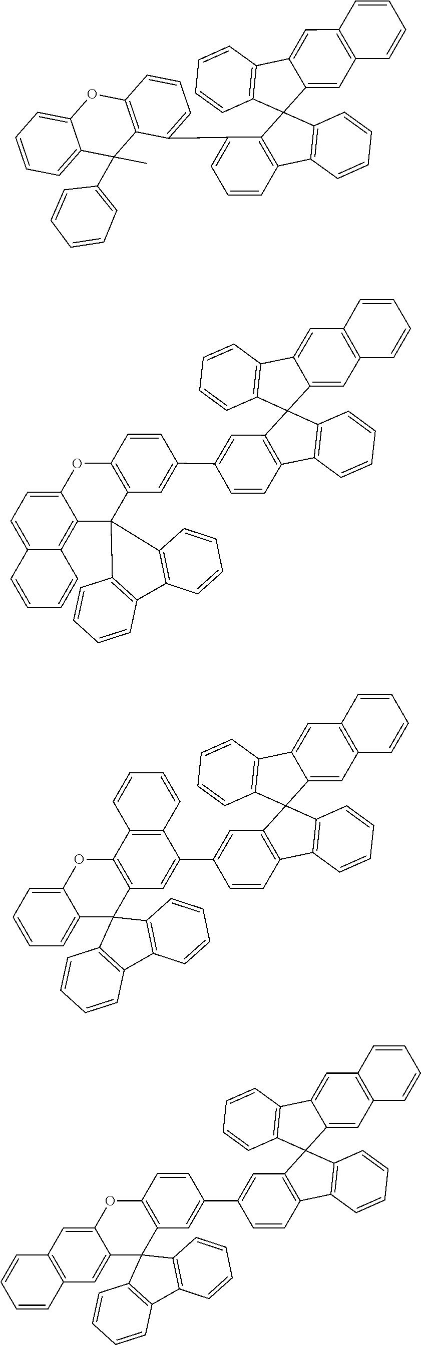 [SMILES: CC1(c2ccccc2)c2ccccc2Oc2cccc(-c3cccc4c3C3(c5ccccc5-c5cc6ccccc6cc53)c3ccccc3-4)c21 ; c1ccc2c(c1)-c1ccc(-c3ccc4c(c3)C3(c5ccccc5-c5ccccc53)c3c(ccc5ccccc35)O4)cc1C21c2ccccc2-c2cc3ccccc3cc21 ; c1ccc2c(c1)Oc1c(cc(-c3ccc4c(c3)C3(c5ccccc5-4)c4ccccc4-c4cc5ccccc5cc43)c3ccccc13)C21c2ccccc2-c2ccccc21 ; c1ccc2c(c1)-c1ccccc1C21c2cc(-c3ccc4c(c3)C3(c5ccccc5-4)c4ccccc4-c4cc5ccccc5cc43)ccc2Oc2cc3ccccc3cc21]

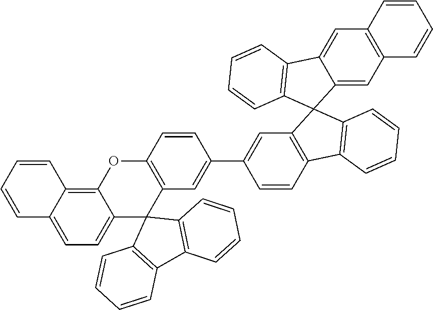 [SMILES: c1ccc2c(c1)-c1ccc(-c3ccc4c(c3)C3(c5ccccc5-c5ccccc53)c3ccc5ccccc5c3O4)cc1C21c2ccccc2-c2cc3ccccc3cc21]

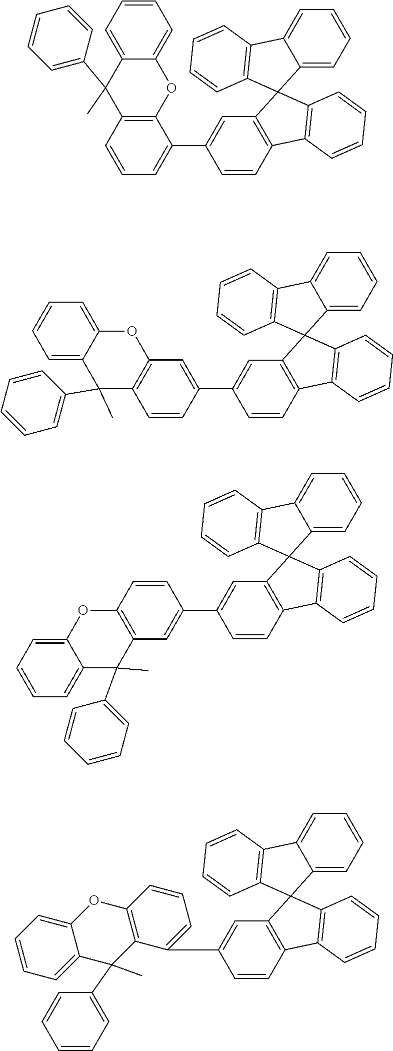 [SMILES: CC1(c2ccccc2)c2ccccc2Oc2c(-c3ccc4c(c3)C3(c5ccccc5-c5ccccc53)c3ccccc3-4)cccc21 ; CC1(c2ccccc2)c2ccccc2Oc2cc(-c3ccc4c(c3)C3(c5ccccc5-c5ccccc53)c3ccccc3-4)ccc21 ; CC1(c2ccccc2)c2ccccc2Oc2ccc(-c3ccc4c(c3)C3(c5ccccc5-c5ccccc53)c3ccccc3-4)cc21 ; CC1(c2ccccc2)c2ccccc2Oc2cccc(-c3ccc4c(c3)C3(c5ccccc5-c5ccccc53)c3ccccc3-4)c21]

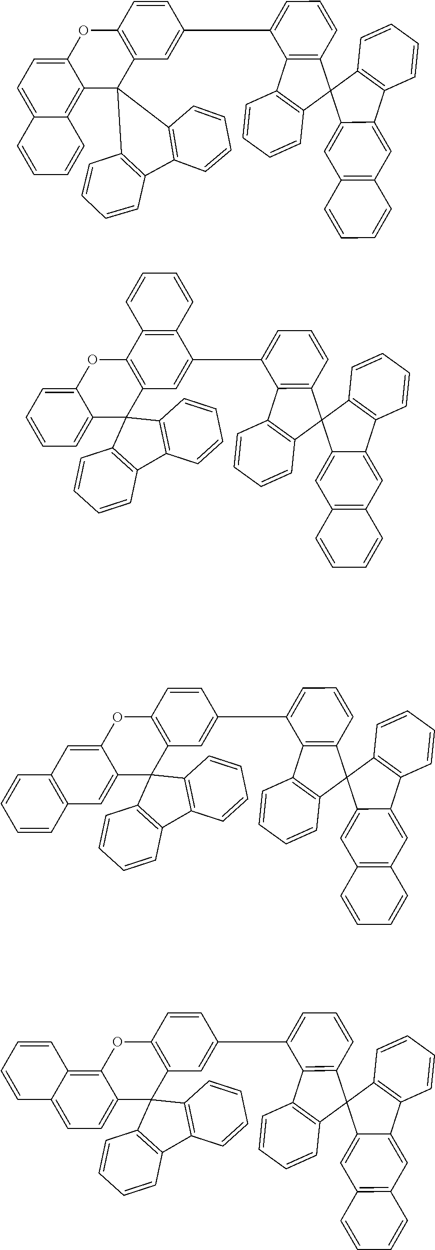 [SMILES: c1ccc2c(c1)-c1cc3ccccc3cc1C21c2ccccc2-c2c(-c3ccc4c(c3)C3(c5ccccc5-c5ccccc53)c3c(ccc5ccccc35)O4)cccc21 ; c1ccc2c(c1)Oc1c(cc(-c3cccc4c3-c3ccccc3C43c4ccccc4-c4cc5ccccc5cc43)c3ccccc13)C21c2ccccc2-c2ccccc21 ; c1ccc2c(c1)-c1ccccc1C21c2cc(-c3cccc4c3-c3ccccc3C43c4ccccc4-c4cc5ccccc5cc43)ccc2Oc2cc3ccccc3cc21 ; c1ccc2c(c1)-c1cc3ccccc3cc1C21c2ccccc2-c2c(-c3ccc4c(c3)C3(c5ccccc5-c5ccccc53)c3ccc5ccccc5c3O4)cccc21]

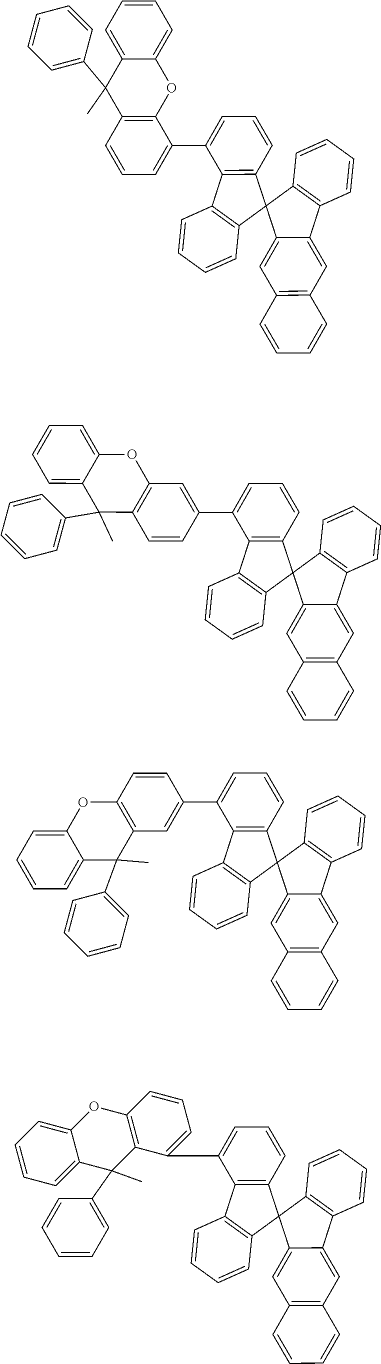 [SMILES: CC1(c2ccccc2)c2ccccc2Oc2c(-c3cccc4c3-c3ccccc3C43c4ccccc4-c4cc5ccccc5cc43)cccc21 ; CC1(c2ccccc2)c2ccccc2Oc2cc(-c3cccc4c3-c3ccccc3C43c4ccccc4-c4cc5ccccc5cc43)ccc21 ; CC1(c2ccccc2)c2ccccc2Oc2ccc(-c3cccc4c3-c3ccccc3C43c4ccccc4-c4cc5ccccc5cc43)cc21 ; CC1(c2ccccc2)c2ccccc2Oc2cccc(-c3cccc4c3-c3ccccc3C43c4ccccc4-c4cc5ccccc5cc43)c21]

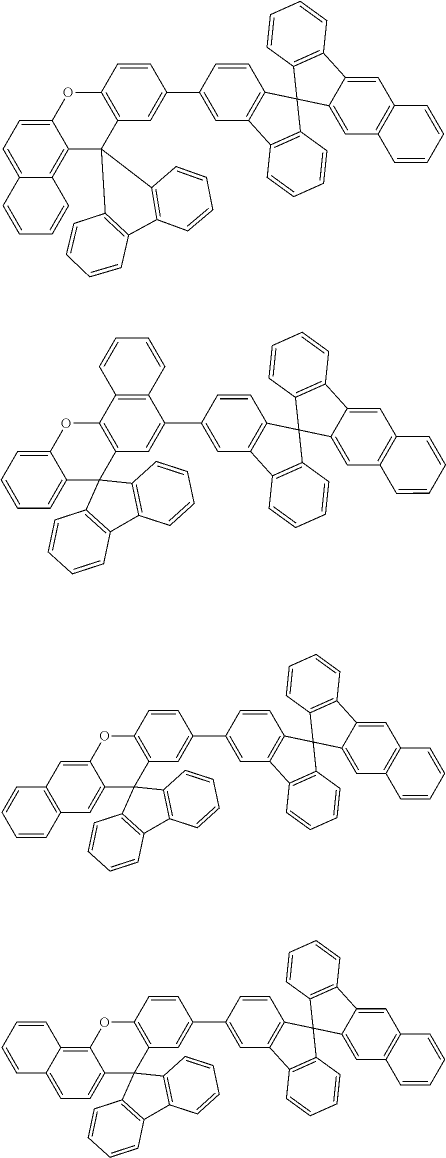 [SMILES: c1ccc2c(c1)-c1cc(-c3ccc4c(c3)C3(c5ccccc5-c5ccccc53)c3c(ccc5ccccc35)O4)ccc1C21c2ccccc2-c2cc3ccccc3cc21 ; c1ccc2c(c1)Oc1c(cc(-c3ccc4c(c3)-c3ccccc3C43c4ccccc4-c4cc5ccccc5cc43)c3ccccc13)C21c2ccccc2-c2ccccc21 ; c1ccc2c(c1)-c1ccccc1C21c2cc(-c3ccc4c(c3)-c3ccccc3C43c4ccccc4-c4cc5ccccc5cc43)ccc2Oc2cc3ccccc3cc21 ; c1ccc2c(c1)-c1cc(-c3ccc4c(c3)C3(c5ccccc5-c5ccccc53)c3ccc5ccccc5c3O4)ccc1C21c2ccccc2-c2cc3ccccc3cc21]

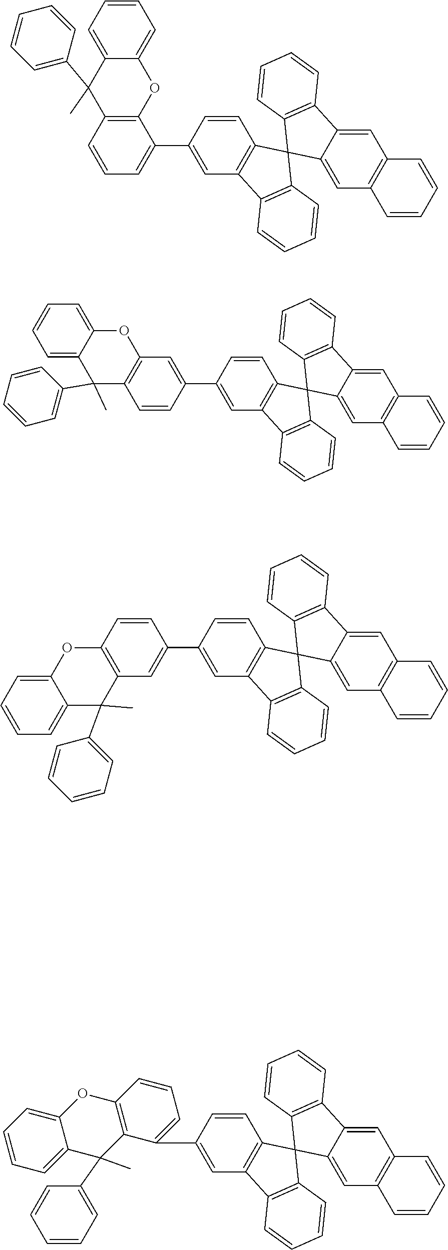 [SMILES: CC1(c2ccccc2)c2ccccc2Oc2c(-c3ccc4c(c3)-c3ccccc3C43c4ccccc4-c4cc5ccccc5cc43)cccc21 ; CC1(c2ccccc2)c2ccccc2Oc2cc(-c3ccc4c(c3)-c3ccccc3C43c4ccccc4-c4cc5ccccc5cc43)ccc21 ; CC1(c2ccccc2)c2ccccc2Oc2ccc(-c3ccc4c(c3)-c3ccccc3C43c4ccccc4-c4cc5ccccc5cc43)cc21 ; CC1(c2ccccc2)c2ccccc2Oc2cccc(-c3ccc4c(c3)-c3ccccc3C43c4ccccc4-c4cc5ccccc5cc43)c21]

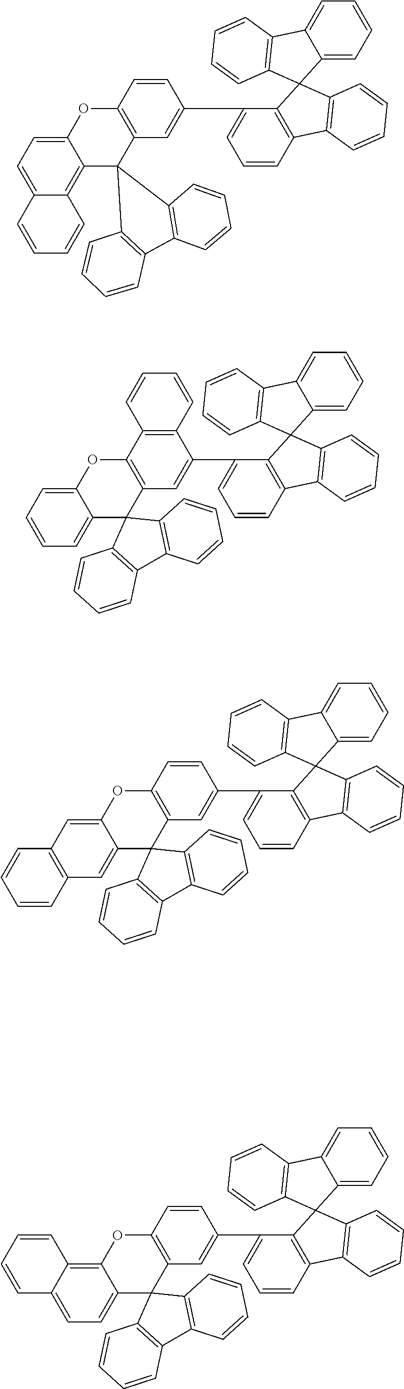 [SMILES: c1ccc2c(c1)-c1ccccc1C21c2ccccc2-c2cccc(-c3ccc4c(c3)C3(c5ccccc5-c5ccccc53)c3c(ccc5ccccc35)O4)c21 ; c1ccc2c(c1)Oc1c(cc(-c3cccc4c3C3(c5ccccc5-c5ccccc53)c3ccccc3-4)c3ccccc13)C21c2ccccc2-c2ccccc21 ; c1ccc2c(c1)-c1ccccc1C21c2cc(-c3cccc4c3C3(c5ccccc5-c5ccccc53)c3ccccc3-4)ccc2Oc2cc3ccccc3cc21 ; c1ccc2c(c1)-c1ccccc1C21c2cc(-c3cccc4c3C3(c5ccccc5-c5ccccc53)c3ccccc3-4)ccc2Oc2c1ccc1ccccc21]

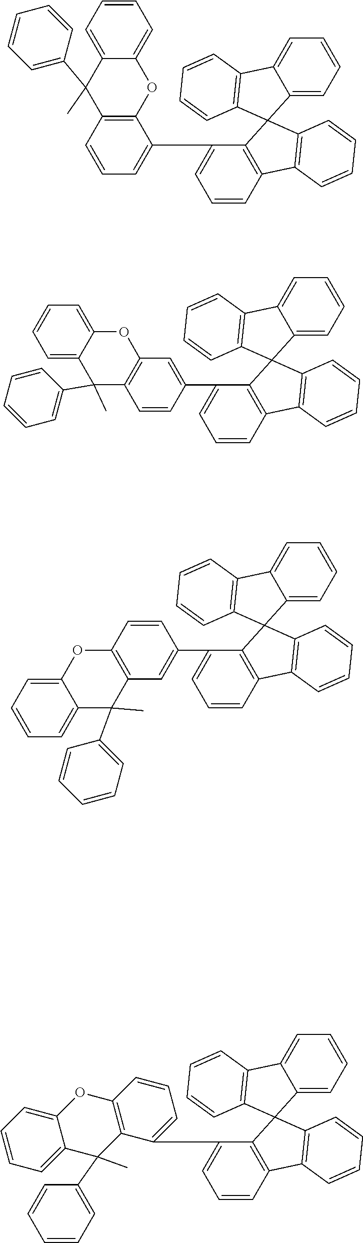 [SMILES: CC1(c2ccccc2)c2ccccc2Oc2c(-c3cccc4c3C3(c5ccccc5-c5ccccc53)c3ccccc3-4)cccc21 ; CC1(c2ccccc2)c2ccccc2Oc2cc(-c3cccc4c3C3(c5ccccc5-c5ccccc53)c3ccccc3-4)ccc21 ; CC1(c2ccccc2)c2ccccc2Oc2ccc(-c3cccc4c3C3(c5ccccc5-c5ccccc53)c3ccccc3-4)cc21 ; CC1(c2ccccc2)c2ccccc2Oc2cccc(-c3cccc4c3C3(c5ccccc5-c5ccccc53)c3ccccc3-4)c21]

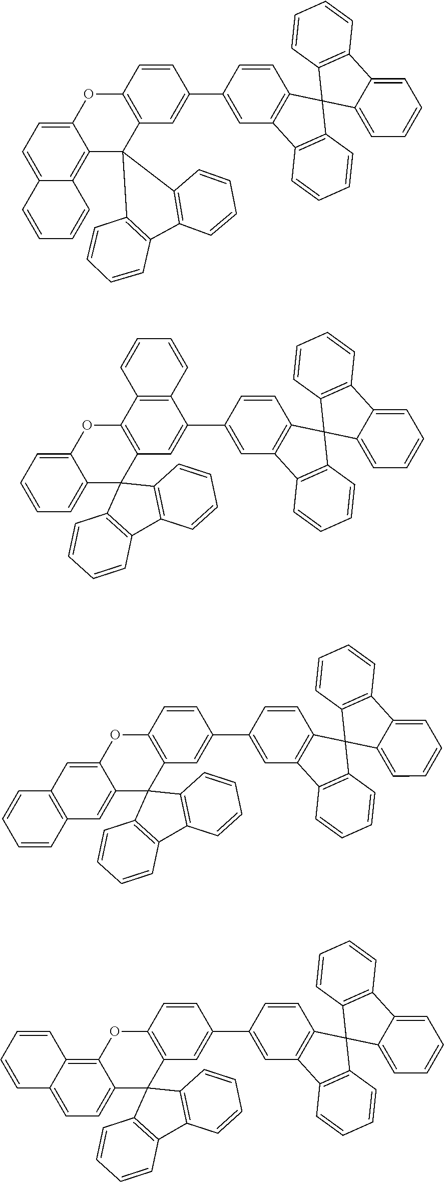 [SMILES: c1ccc2c(c1)-c1ccccc1C21c2ccccc2-c2cc(-c3ccc4c(c3)C3(c5ccccc5-c5ccccc53)c3c(ccc5ccccc35)O4)ccc21 ; c1ccc2c(c1)Oc1c(cc(-c3ccc4c(c3)-c3ccccc3C43c4ccccc4-c4ccccc43)c3ccccc13)C21c2ccccc2-c2ccccc21 ; c1ccc2c(c1)-c1ccccc1C21c2cc(-c3ccc4c(c3)-c3ccccc3C43c4ccccc4-c4ccccc43)ccc2Oc2cc3ccccc3cc21 ; c1ccc2c(c1)-c1ccccc1C21c2ccccc2-c2cc(-c3ccc4c(c3)C3(c5ccccc5-c5ccccc53)c3ccc5ccccc5c3O4)ccc21]

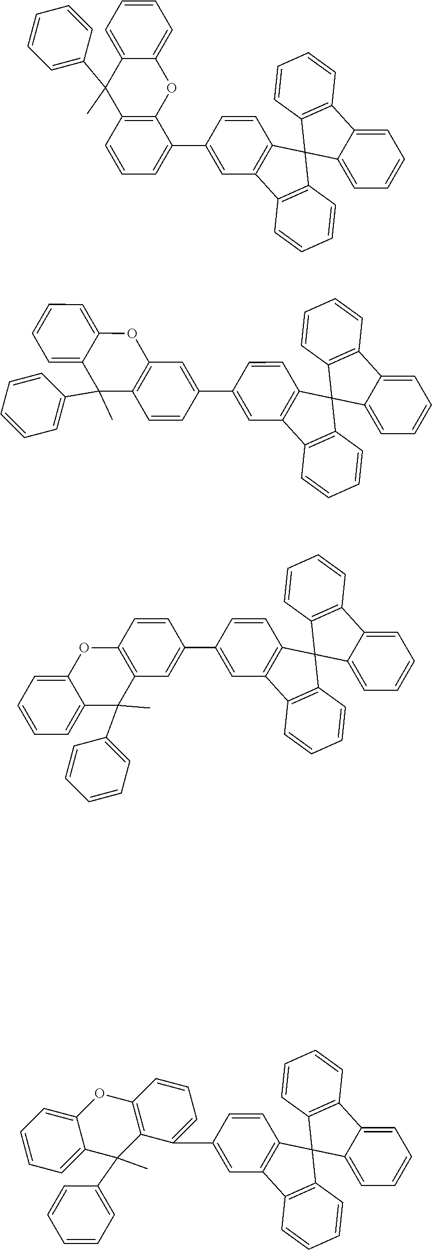 [SMILES: CC1(c2ccccc2)c2ccccc2Oc2c(-c3ccc4c(c3)-c3ccccc3C43c4ccccc4-c4ccccc43)cccc21 ; CC1(c2ccccc2)c2ccccc2Oc2cc(-c3ccc4c(c3)-c3ccccc3C43c4ccccc4-c4ccccc43)ccc21 ; CC1(c2ccccc2)c2ccccc2Oc2ccc(-c3ccc4c(c3)-c3ccccc3C43c4ccccc4-c4ccccc43)cc21 ; CC1(c2ccccc2)c2ccccc2Oc2cccc(-c3ccc4c(c3)-c3ccccc3C43c4ccccc4-c4ccccc43)c21]

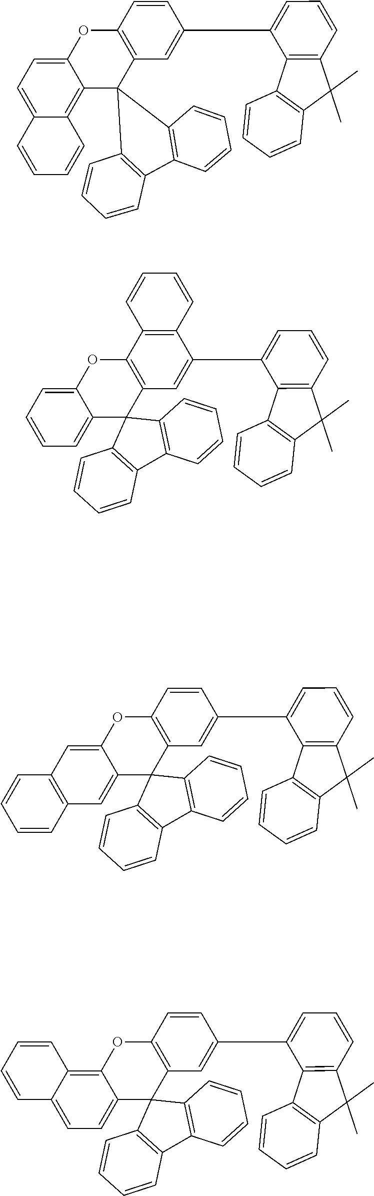 [SMILES: CC1(C)c2ccccc2-c2c(-c3ccc4c(c3)C3(c5ccccc5-c5ccccc53)c3c(ccc5ccccc35)O4)cccc21 ; CC1(C)c2ccccc2-c2c(-c3cc4c(c5ccccc35)Oc3ccccc3C43c4ccccc4-c4ccccc43)cccc21 ; CC1(C)c2ccccc2-c2c(-c3ccc4c(c3)C3(c5cc6ccccc6cc5O4)c4ccccc4-c4ccccc43)cccc21 ; CC1(C)c2ccccc2-c2c(-c3ccc4c(c3)C3(c5ccccc5-c5ccccc53)c3ccc5ccccc5c3O4)cccc21]

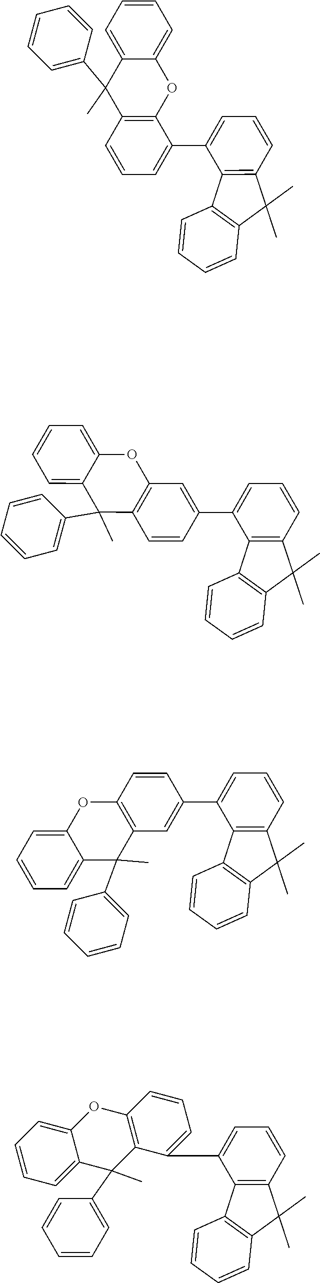 [SMILES: CC1(C)c2ccccc2-c2c(-c3cccc4c3Oc3ccccc3C4(C)c3ccccc3)cccc21 ; CC1(C)c2ccccc2-c2c(-c3ccc4c(c3)Oc3ccccc3C4(C)c3ccccc3)cccc21 ; CC1(C)c2ccccc2-c2c(-c3ccc4c(c3)C(C)(c3ccccc3)c3ccccc3O4)cccc21 ; CC1(C)c2ccccc2-c2c(-c3cccc4c3C(C)(c3ccccc3)c3ccccc3O4)cccc21]

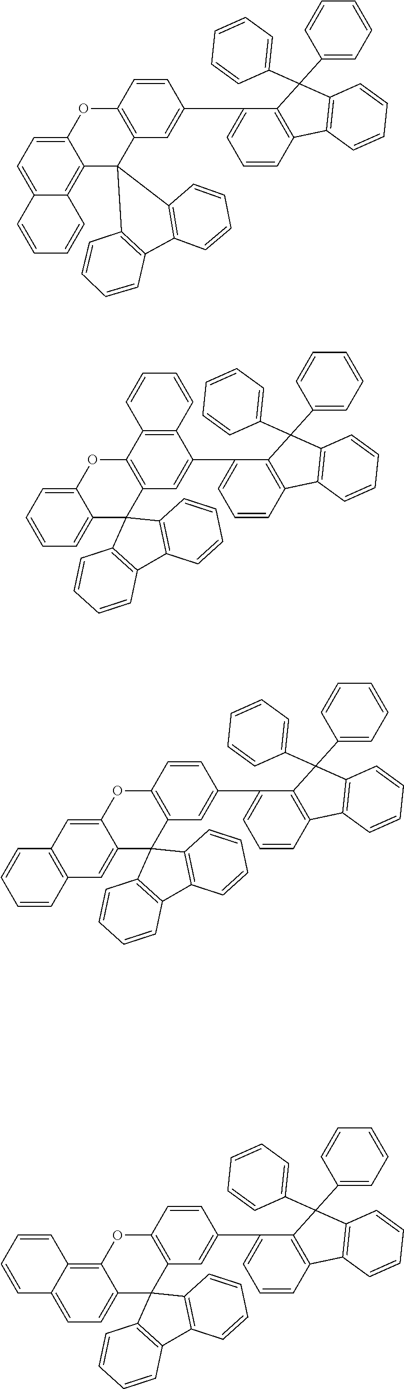 [SMILES: c1ccc(C2(c3ccccc3)c3ccccc3-c3cccc(-c4ccc5c(c4)C4(c6ccccc6-c6ccccc64)c4c(ccc6ccccc46)O5)c32)cc1 ; c1ccc(C2(c3ccccc3)c3ccccc3-c3cccc(-c4cc5c(c6ccccc46)Oc4ccccc4C54c5ccccc5-c5ccccc54)c32)cc1 ; c1ccc(C2(c3ccccc3)c3ccccc3-c3cccc(-c4ccc5c(c4)C4(c6cc7ccccc7cc6O5)c5ccccc5-c5ccccc54)c32)cc1 ; c1ccc(C2(c3ccccc3)c3ccccc3-c3cccc(-c4ccc5c(c4)C4(c6ccccc6-c6ccccc64)c4ccc6ccccc6c4O5)c32)cc1]

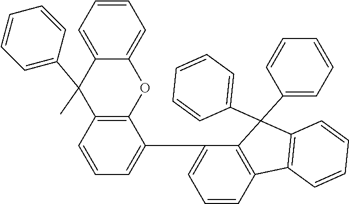 [SMILES: CC1(c2ccccc2)c2ccccc2Oc2c(-c3cccc4c3C(c3ccccc3)(c3ccccc3)c3ccccc3-4)cccc21]

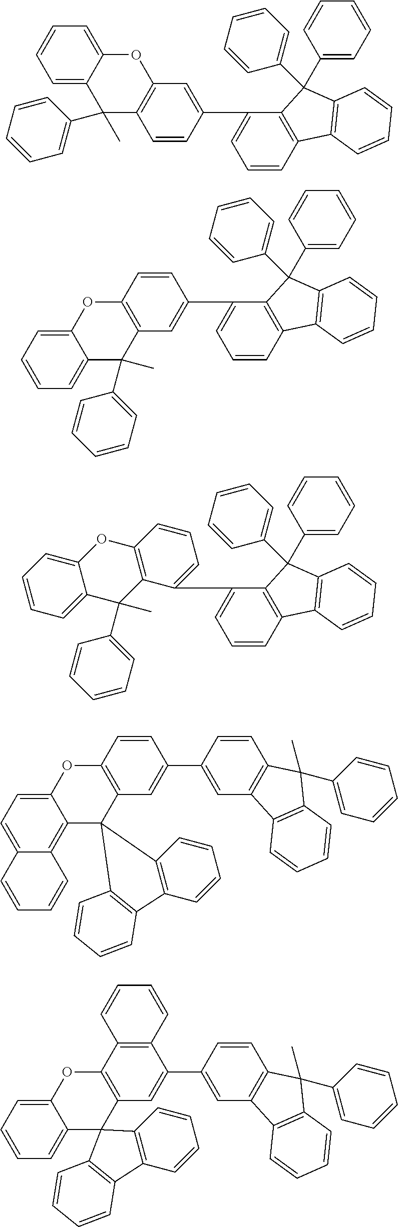

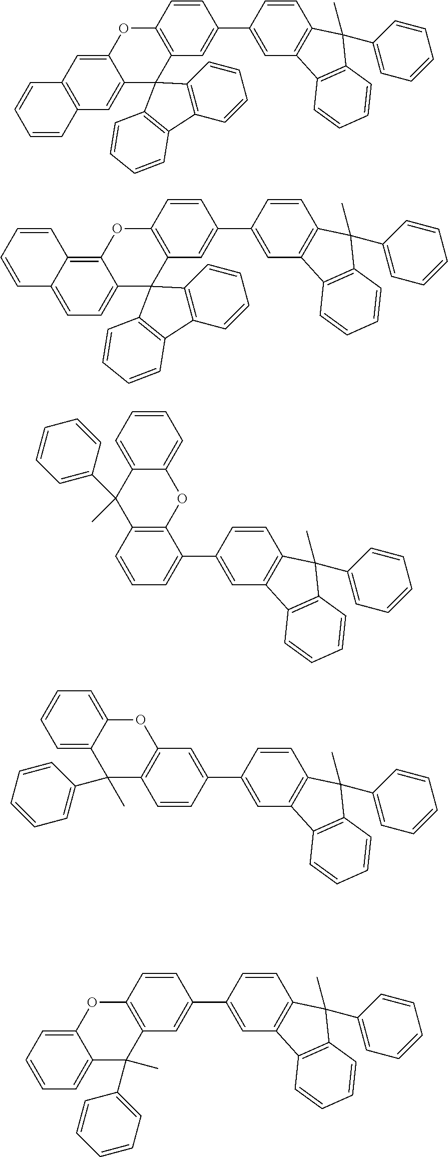

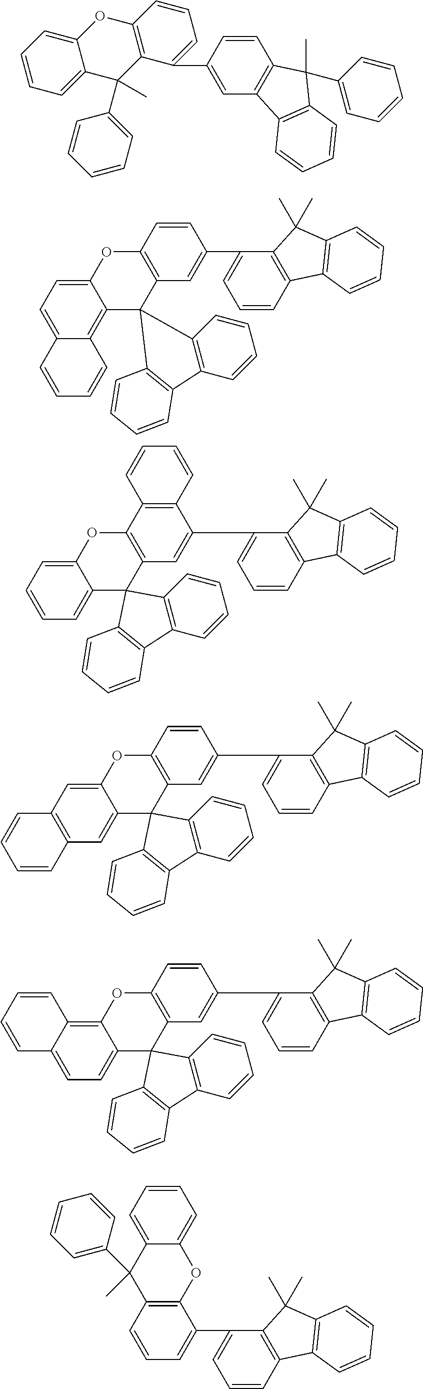

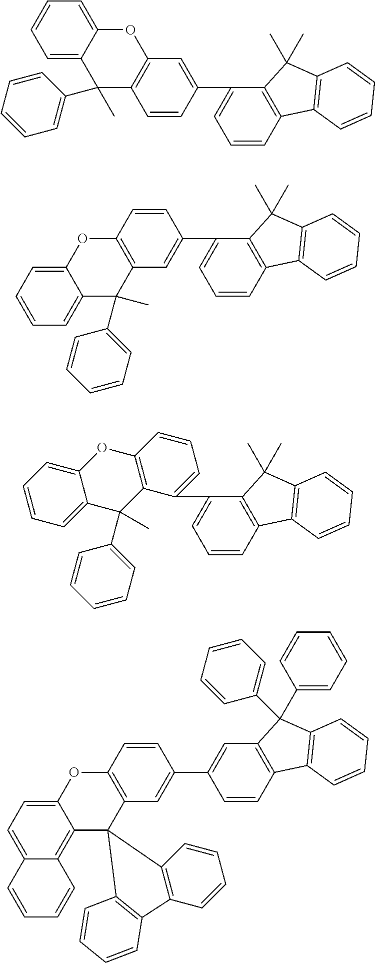

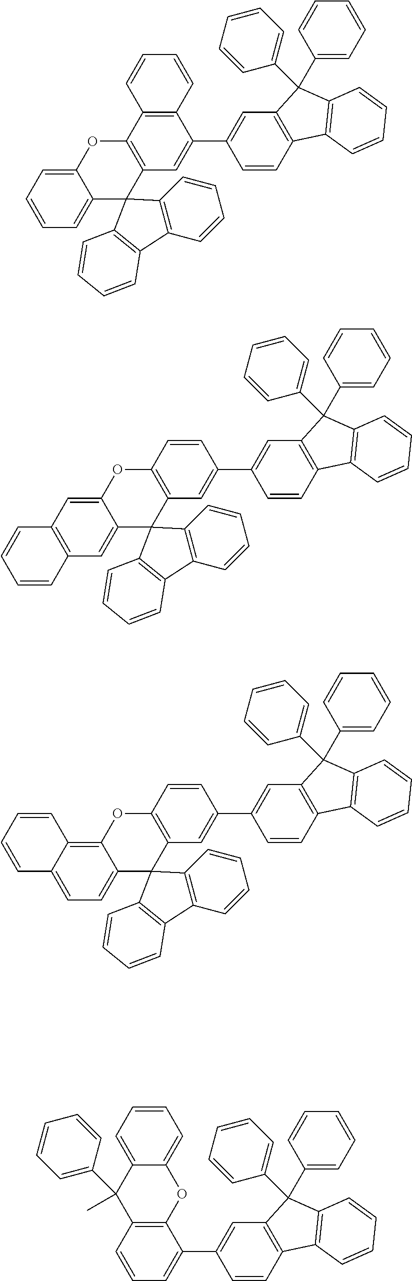 [SMILES: c1ccc(C2(c3ccccc3)c3ccccc3-c3ccc(-c4cc5c(c6ccccc46)Oc4ccccc4C54c5ccccc5-c5ccccc54)cc32)cc1 ; c1ccc(C2(c3ccccc3)c3ccccc3-c3ccc(-c4ccc5c(c4)C4(c6cc7ccccc7cc6O5)c5ccccc5-c5ccccc54)cc32)cc1 ; c1ccc(C2(c3ccccc3)c3ccccc3-c3ccc(-c4ccc5c(c4)C4(c6ccccc6-c6ccccc64)c4ccc6ccccc6c4O5)cc32)cc1 ; CC1(c2ccccc2)c2ccccc2Oc2c(-c3ccc4c(c3)C(c3ccccc3)(c3ccccc3)c3ccccc3-4)cccc21]

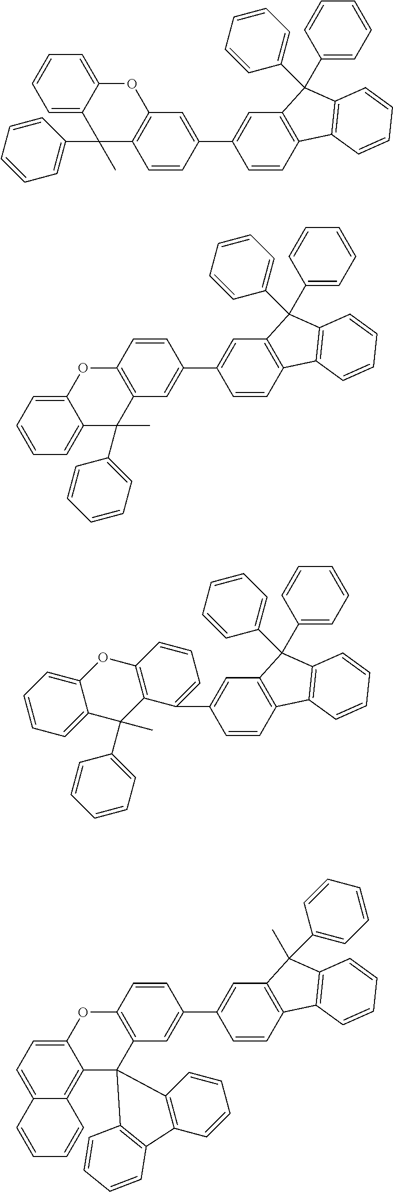 [SMILES: CC1(c2ccccc2)c2ccccc2Oc2cc(-c3ccc4c(c3)C(c3ccccc3)(c3ccccc3)c3ccccc3-4)ccc21 ; CC1(c2ccccc2)c2ccccc2Oc2ccc(-c3ccc4c(c3)C(c3ccccc3)(c3ccccc3)c3ccccc3-4)cc21 ; CC1(c2ccccc2)c2ccccc2Oc2cccc(-c3ccc4c(c3)C(c3ccccc3)(c3ccccc3)c3ccccc3-4)c21 ; CC1(c2ccccc2)c2ccccc2-c2ccc(-c3ccc4c(c3)C3(c5ccccc5-c5ccccc53)c3c(ccc5ccccc35)O4)cc21]

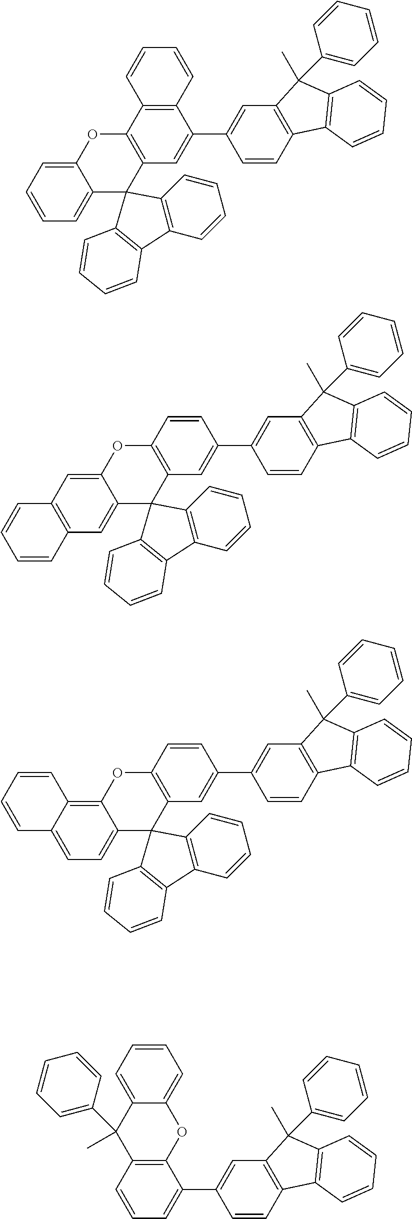 [SMILES: CC1(c2ccccc2)c2ccccc2-c2ccc(-c3cc4c(c5ccccc35)Oc3ccccc3C43c4ccccc4-c4ccccc43)cc21 ; CC1(c2ccccc2)c2ccccc2-c2ccc(-c3ccc4c(c3)C3(c5cc6ccccc6cc5O4)c4ccccc4-c4ccccc43)cc21 ; CC1(c2ccccc2)c2ccccc2-c2ccc(-c3ccc4c(c3)C3(c5ccccc5-c5ccccc53)c3ccc5ccccc5c3O4)cc21 ; CC1(c2ccccc2)c2ccccc2-c2ccc(-c3cccc4c3Oc3ccccc3C4(C)c3ccccc3)cc21]

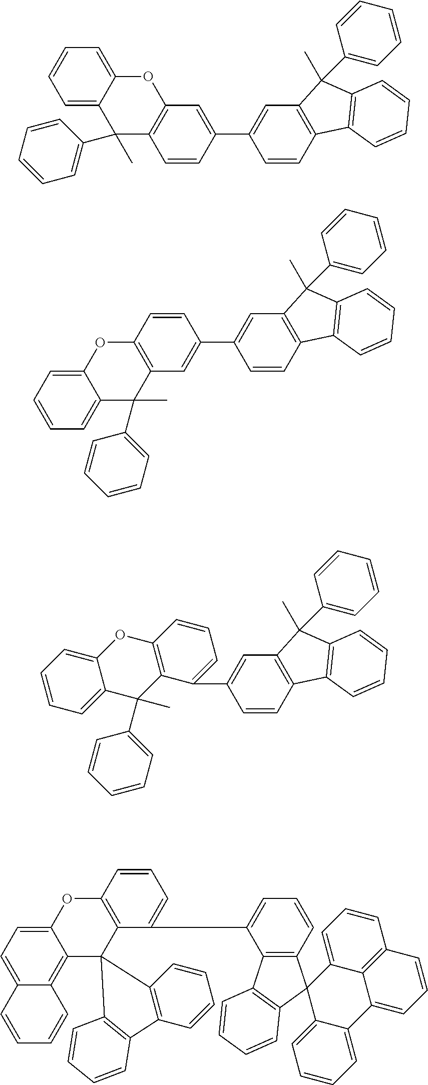 [SMILES: CC1(c2ccccc2)c2ccccc2Oc2cc(-c3ccc4c(c3)C(C)(c3ccccc3)c3ccccc3-4)ccc21 ; CC1(c2ccccc2)c2ccccc2Oc2ccc(-c3ccc4c(c3)C(C)(c3ccccc3)c3ccccc3-4)cc21 ; CC1(c2ccccc2)c2ccccc2-c2ccc(-c3cccc4c3C(C)(c3ccccc3)c3ccccc3O4)cc21 ; c1ccc2c(c1)-c1ccccc1C21c2c(cccc2-c2cccc3c2-c2ccccc2C32c3ccccc3-c3cccc4cccc2c34)Oc2ccc3ccccc3c21]

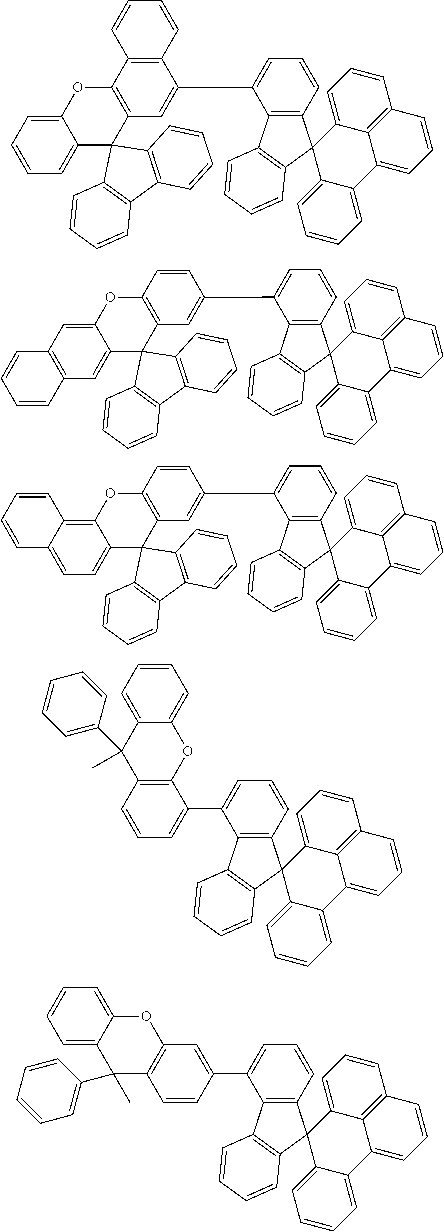

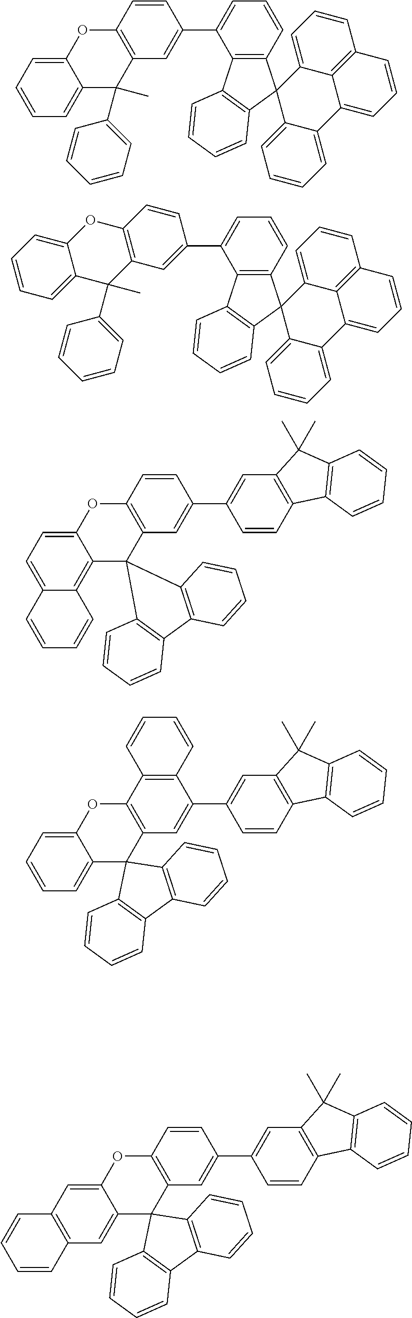

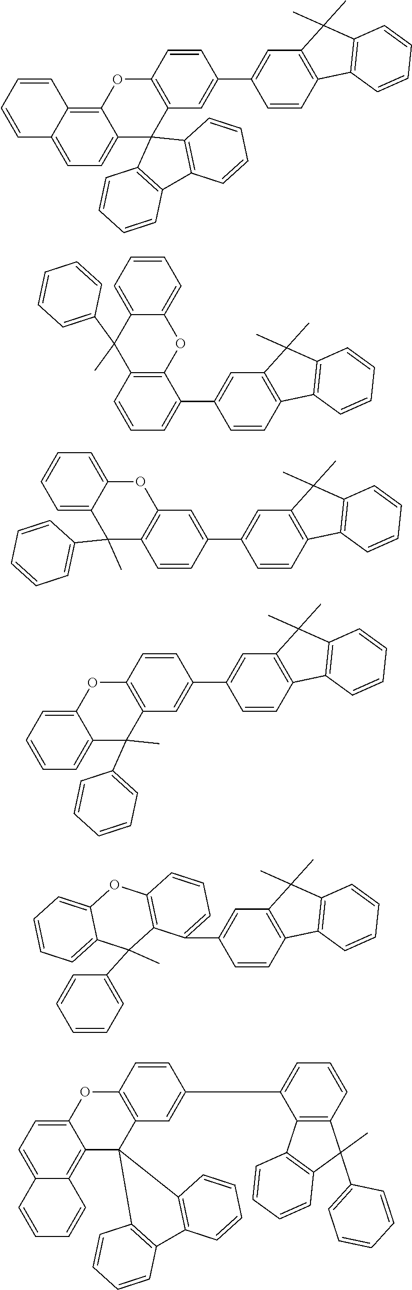

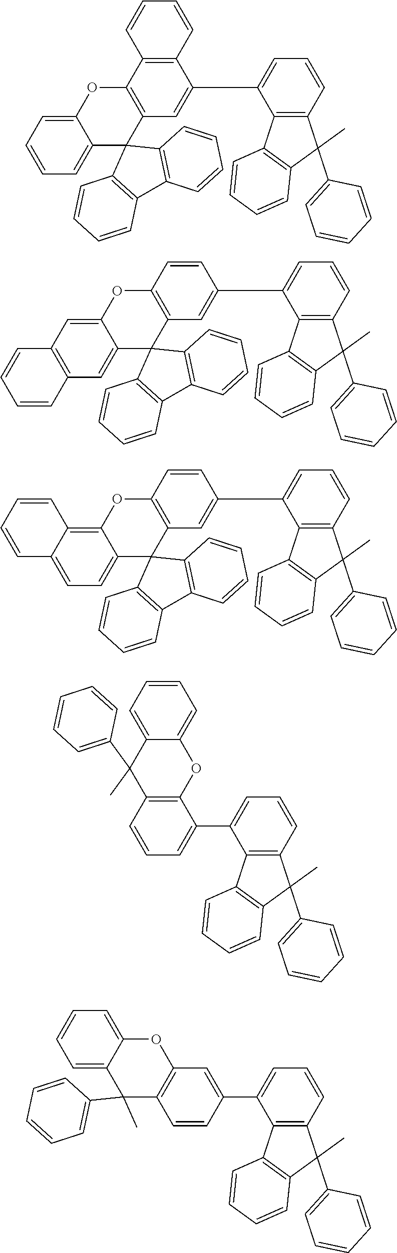

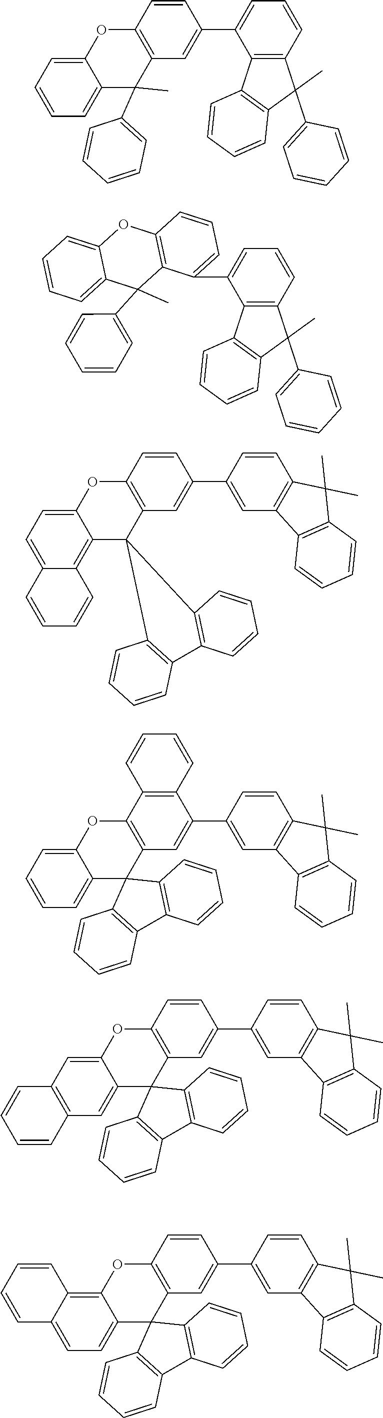

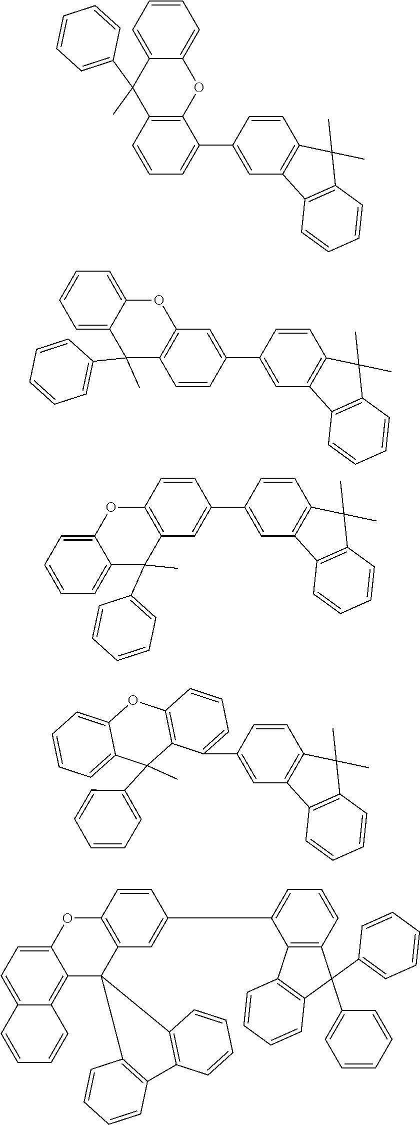

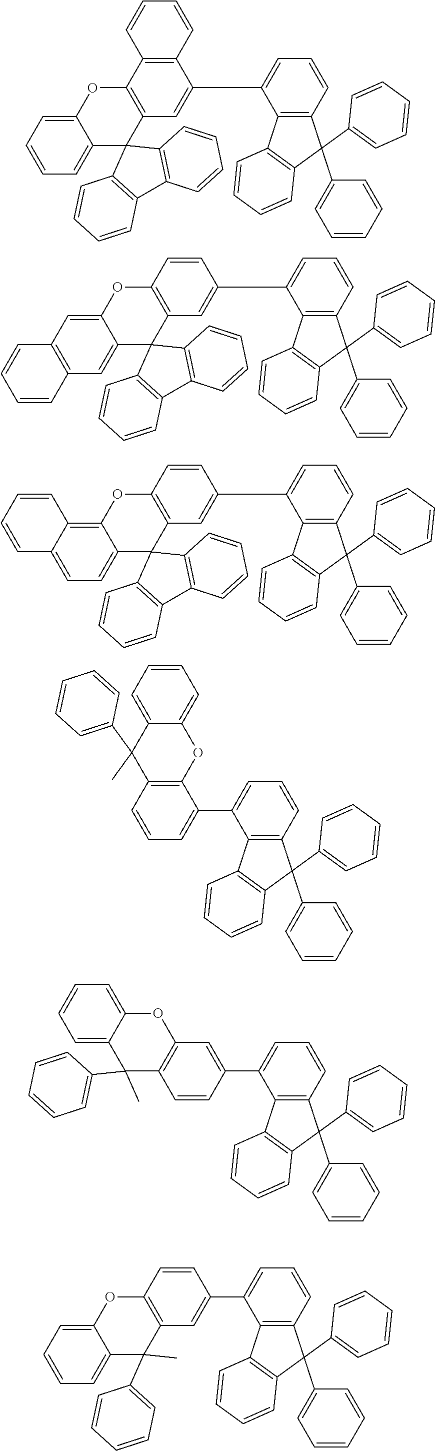

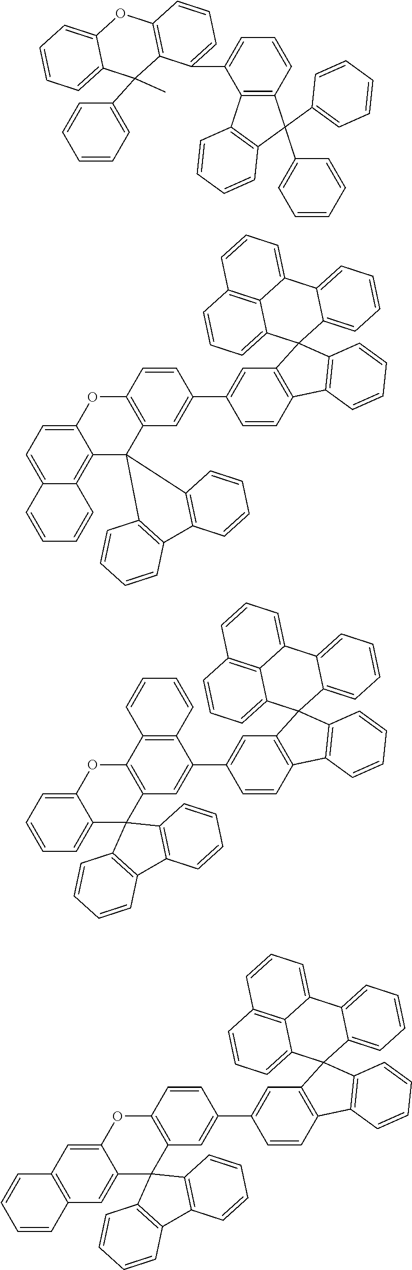

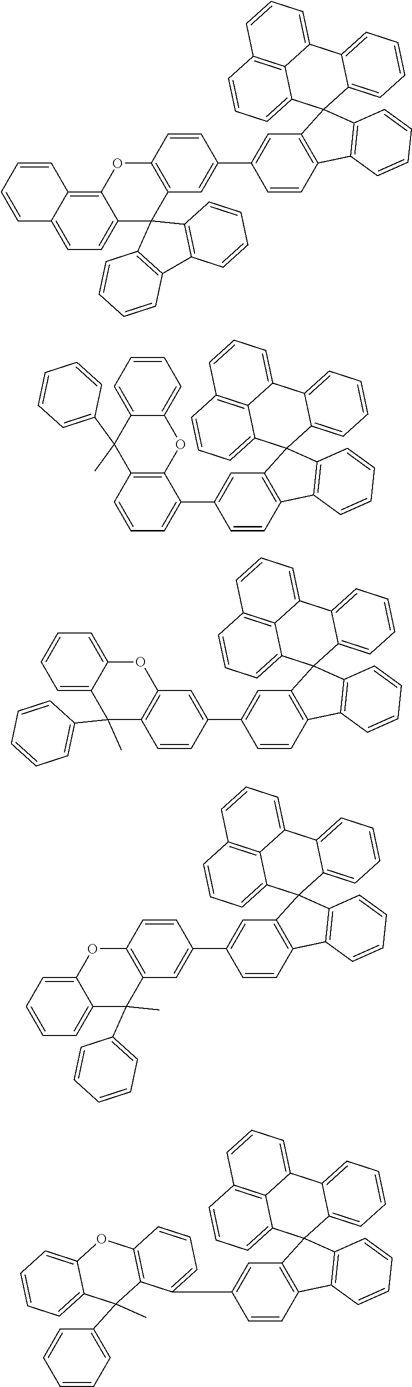

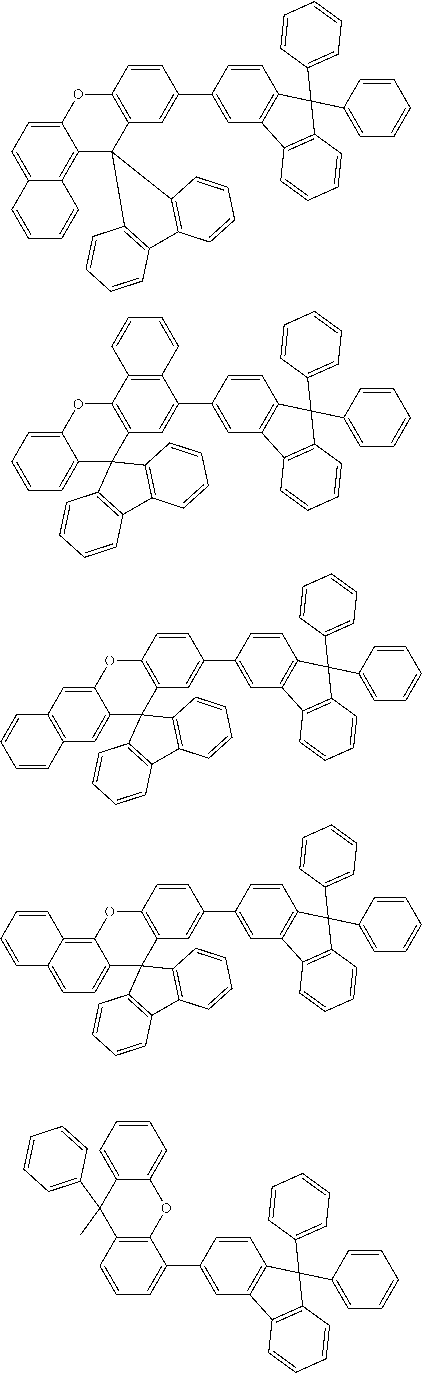

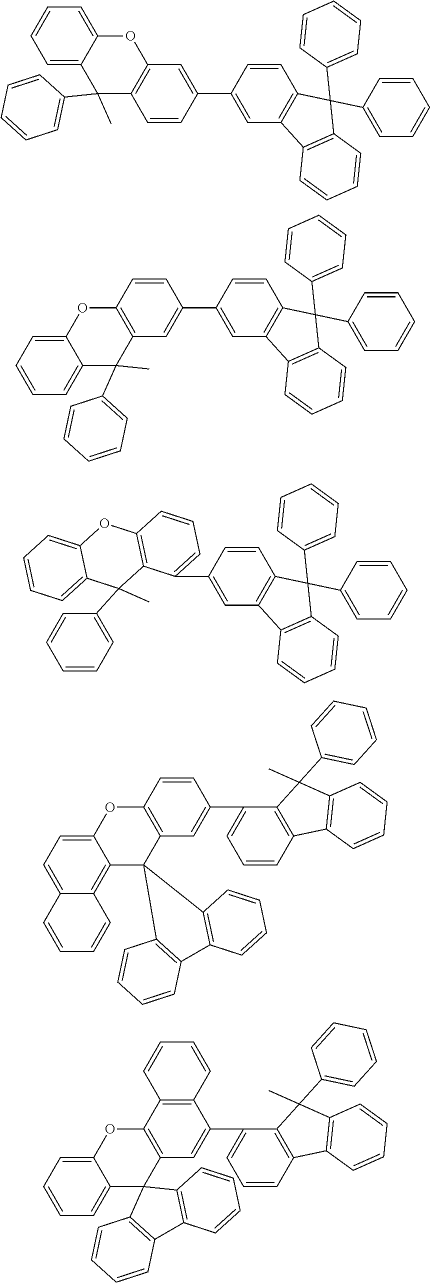

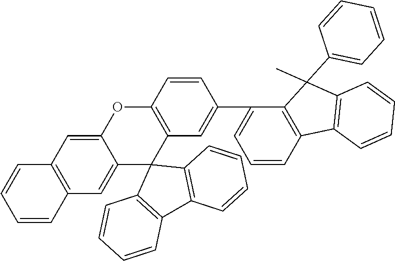 [SMILES: CC1(c2ccccc2)c2ccccc2-c2cccc(-c3ccc4c(c3)C3(c5cc6ccccc6cc5O4)c4ccccc4-c4ccccc43)c21]

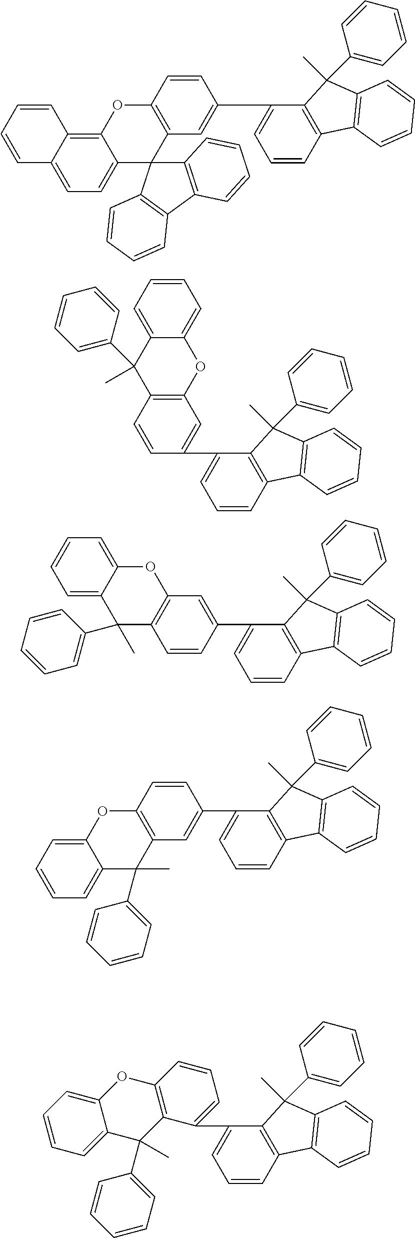

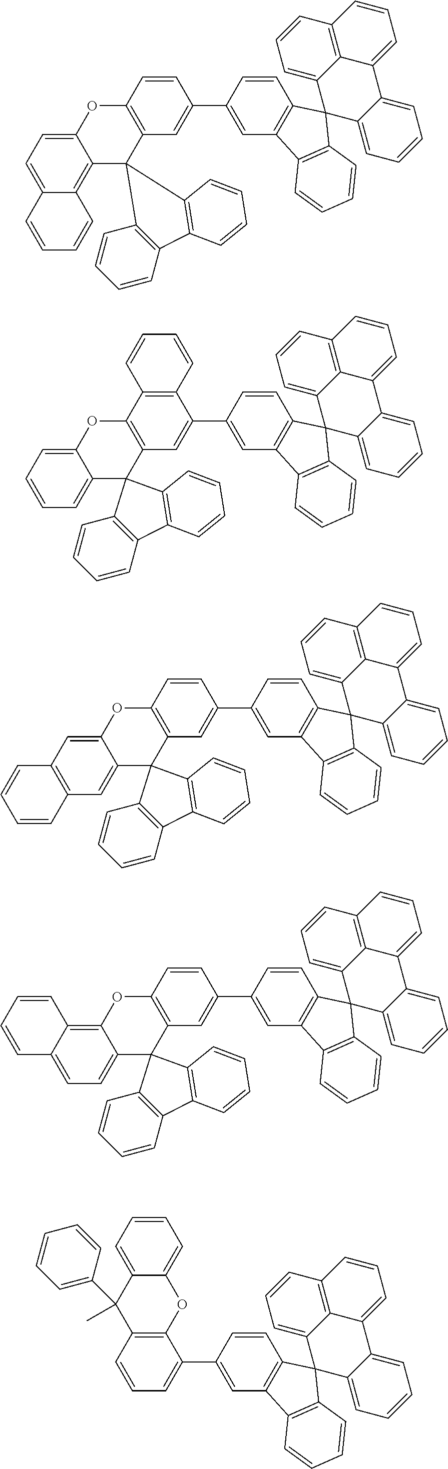 [SMILES: c1ccc2c(c1)-c1ccccc1C21c2cc(-c3ccc4c(c3)-c3ccccc3C43c4ccccc4-c4cccc5cccc3c45)ccc2Oc2ccc3ccccc3c21 ; c1ccc2c(c1)Oc1c(cc(-c3ccc4c(c3)-c3ccccc3C43c4ccccc4-c4cccc5cccc3c45)c3ccccc13)C21c2ccccc2-c2ccccc21 ; c1ccc2c(c1)-c1ccccc1C21c2cc(-c3ccc4c(c3)-c3ccccc3C43c4ccccc4-c4cccc5cccc3c45)ccc2Oc2cc3ccccc3cc21 ; c1ccc2c(c1)-c1ccccc1C21c2cc(-c3ccc4c(c3)-c3ccccc3C43c4ccccc4-c4cccc5cccc3c45)ccc2Oc2c1ccc1ccccc21 ; CC1(c2ccccc2)c2ccccc2Oc2c(-c3ccc4c(c3)-c3ccccc3C43c4ccccc4-c4cccc5cccc3c45)cccc21]

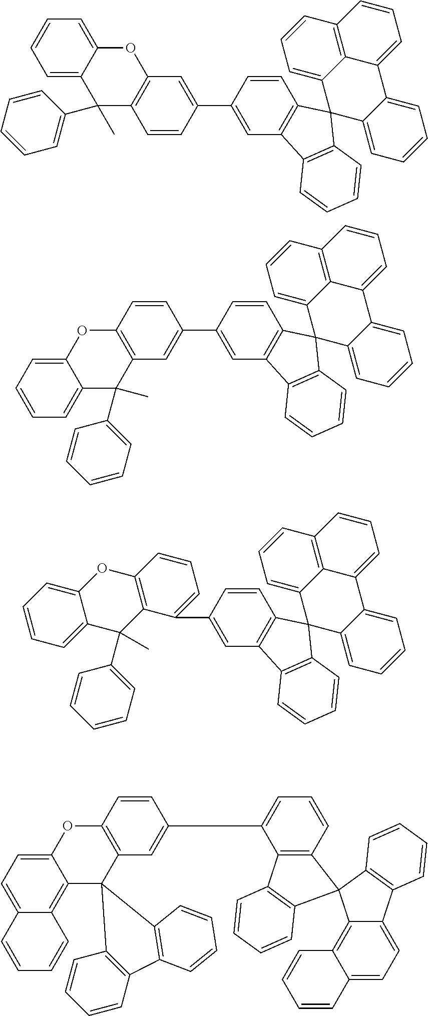

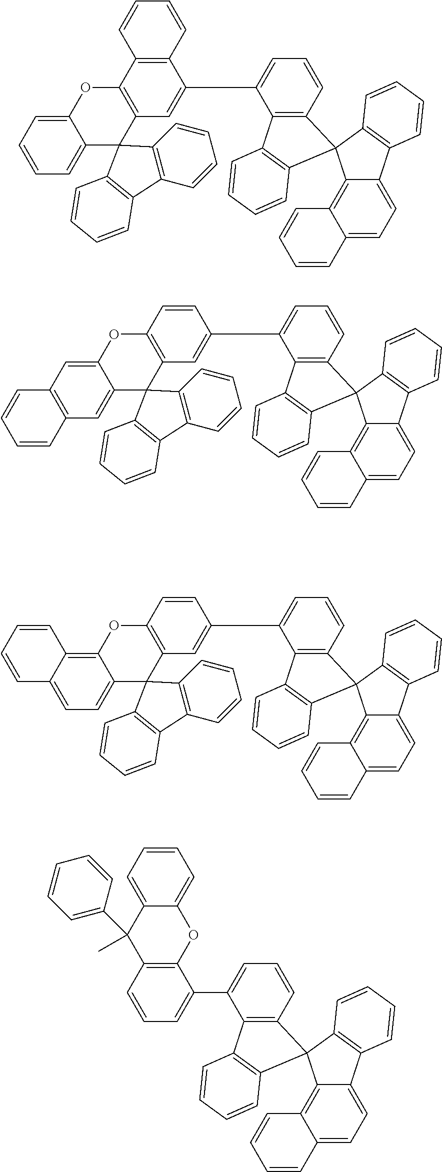

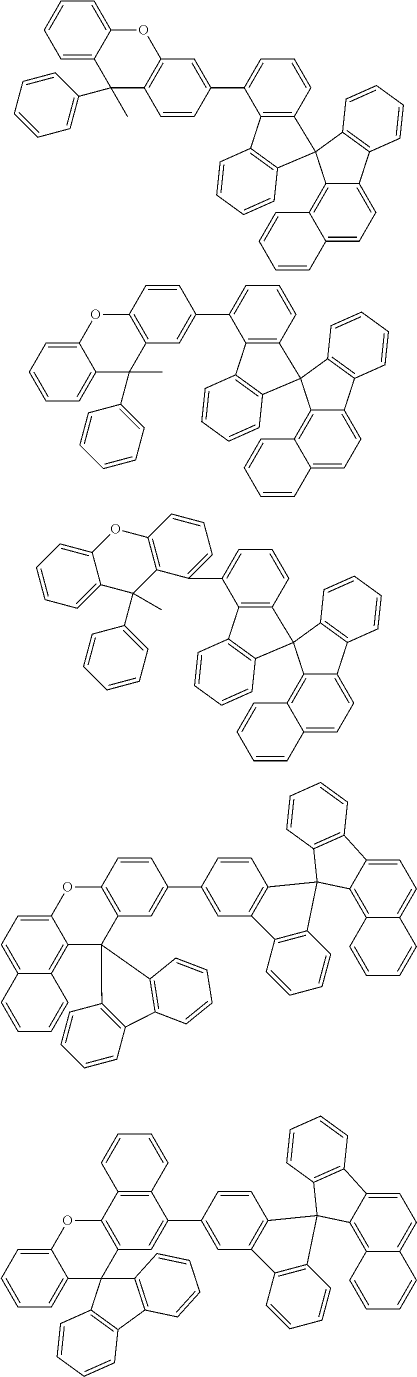

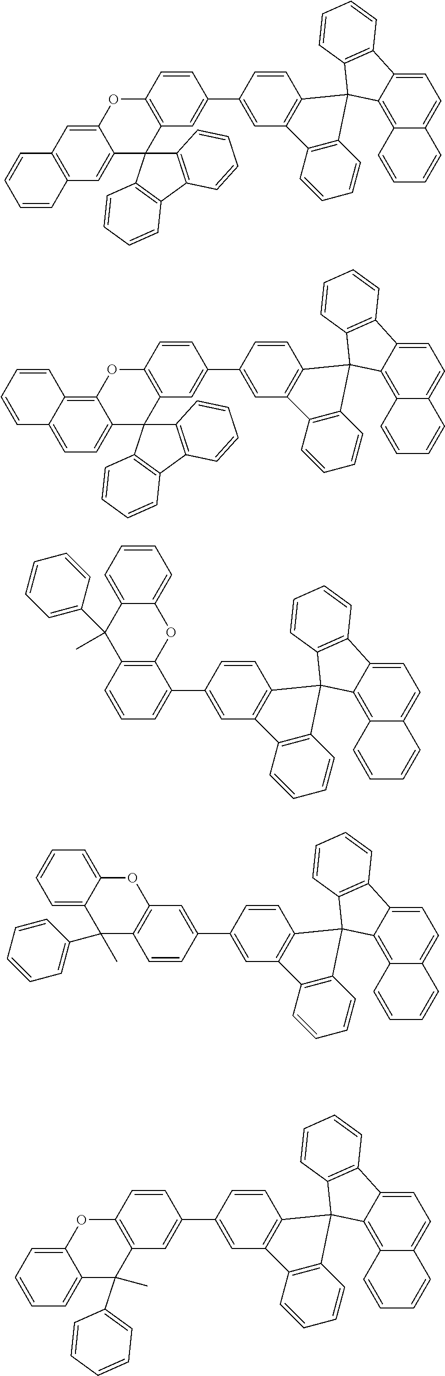

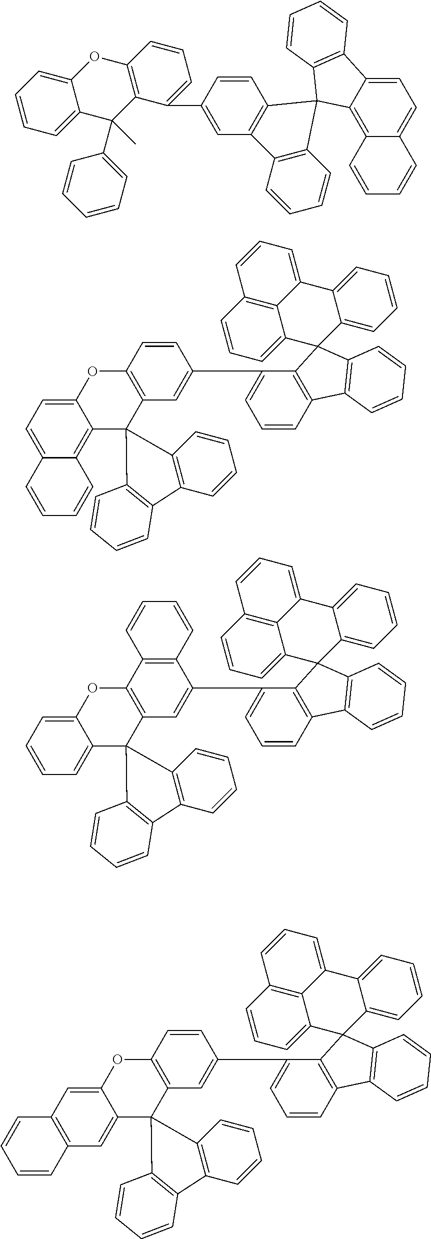

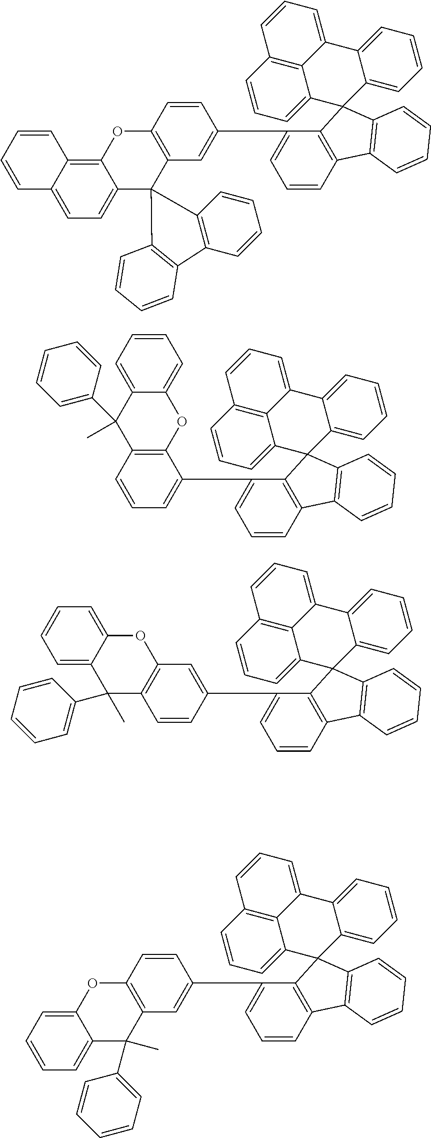

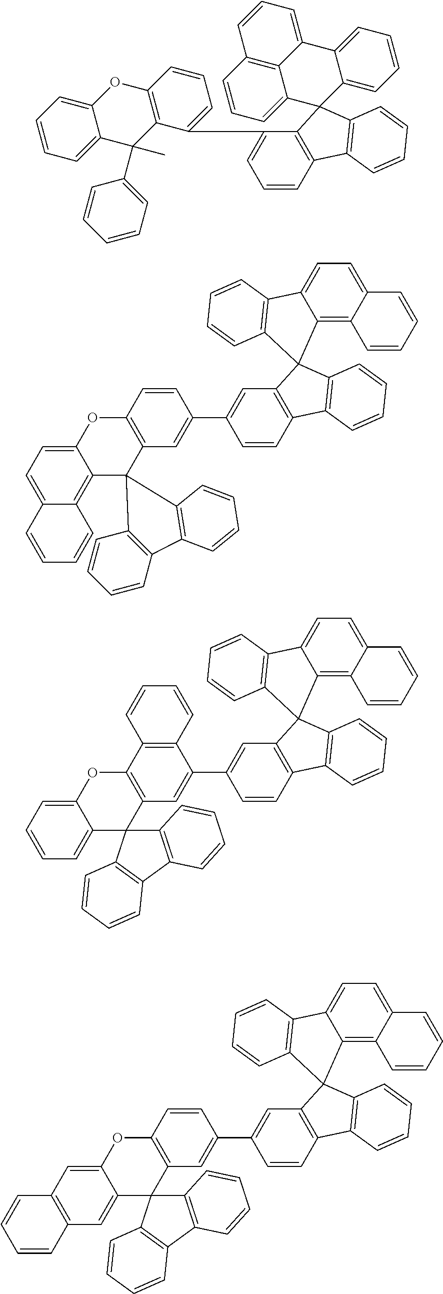 [SMILES: CC1(c2ccccc2)c2ccccc2Oc2cccc(-c3cccc4c3C3(c5ccccc5-4)c4ccccc4-c4cccc5cccc3c45)c21 ; c1ccc2c(c1)-c1ccccc1C21c2cc(-c3ccc4c(c3)C3(c5ccccc5-4)c4ccccc4-c4ccc5ccccc5c43)ccc2Oc2ccc3ccccc3c21 ; c1ccc2c(c1)Oc1c(cc(-c3ccc4c(c3)C3(c5ccccc5-4)c4ccccc4-c4ccc5ccccc5c43)c3ccccc13)C21c2ccccc2-c2ccccc21 ; c1ccc2c(c1)-c1ccccc1C21c2cc(-c3ccc4c(c3)C3(c5ccccc5-4)c4ccccc4-c4ccc5ccccc5c43)ccc2Oc2cc3ccccc3cc21]

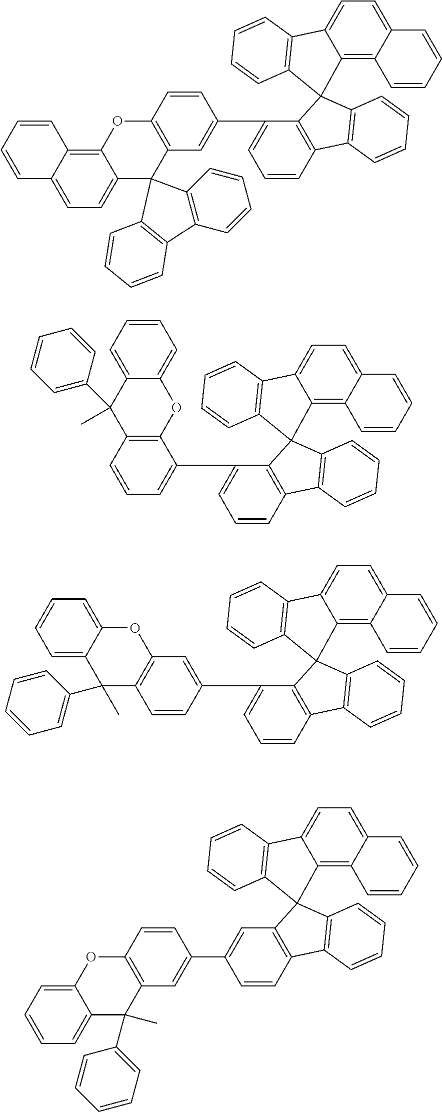

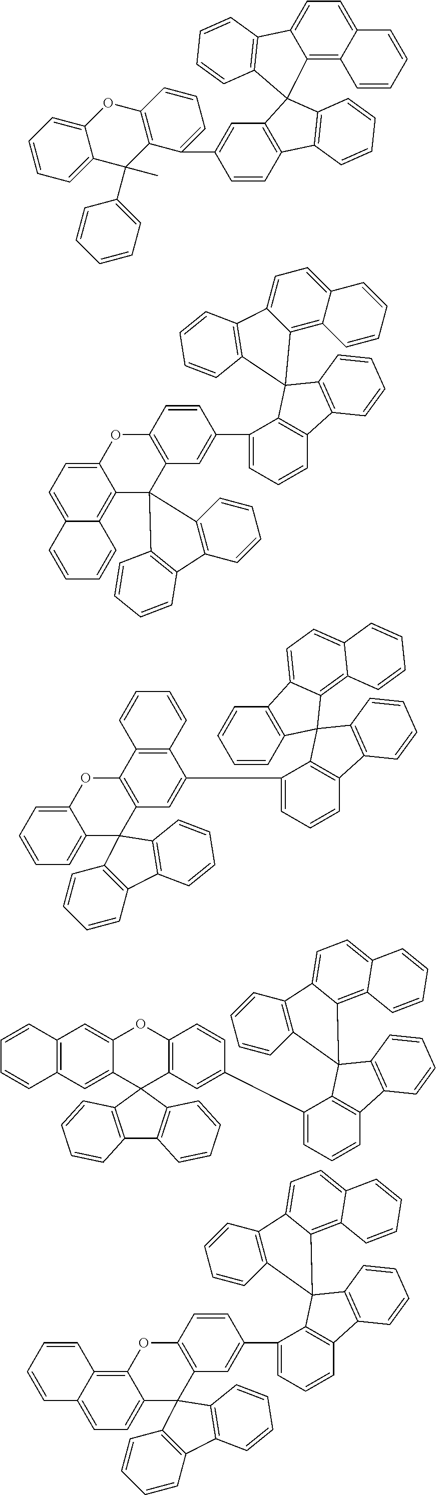

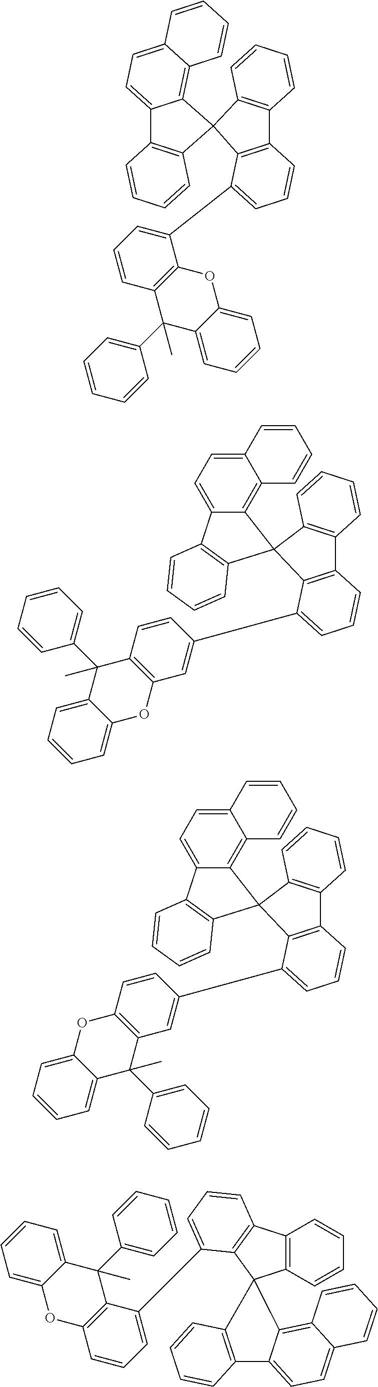 [SMILES: CC1(c2ccccc2)c2ccccc2Oc2c(-c3cccc4c3C3(c5ccccc5-4)c4ccccc4-c4ccc5ccccc5c43)cccc21 ; CC1(c2ccccc2)c2ccccc2Oc2cc(-c3cccc4c3C3(c5ccccc5-4)c4ccccc4-c4ccc5ccccc5c43)ccc21 ; CC1(c2ccccc2)c2ccccc2Oc2ccc(-c3cccc4c3C3(c5ccccc5-4)c4ccccc4-c4ccc5ccccc5c43)cc21 ; CC1(c2ccccc2)c2ccccc2Oc2cccc(-c3cccc4c3C3(c5ccccc5-4)c4ccccc4-c4ccc5ccccc5c43)c21]

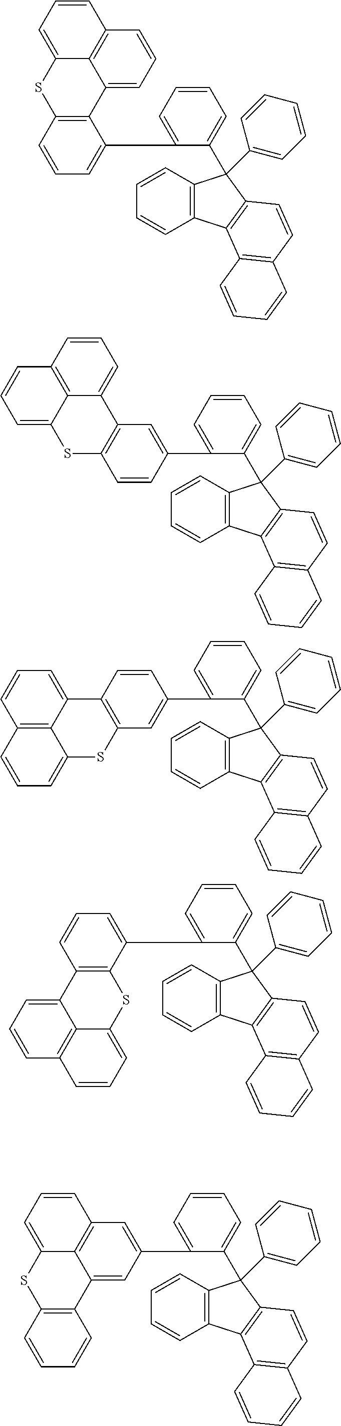

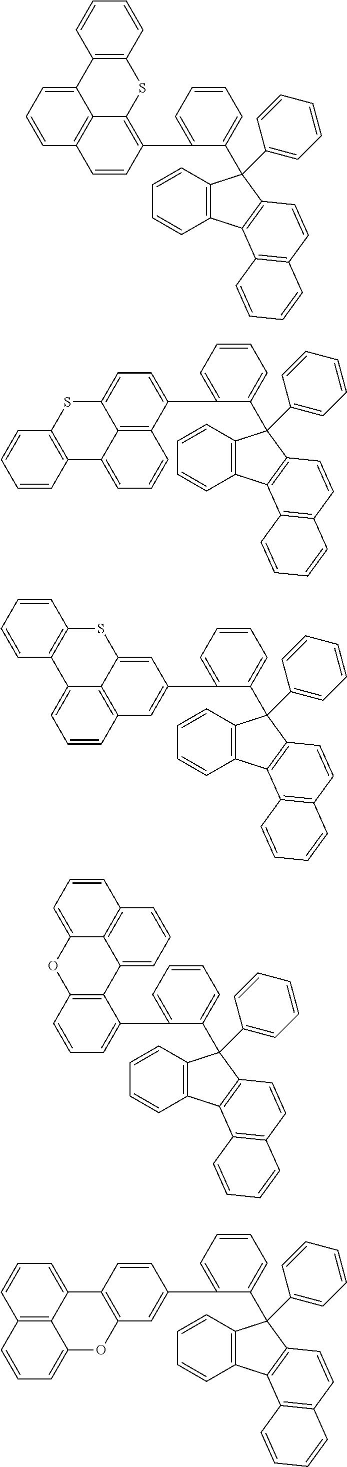

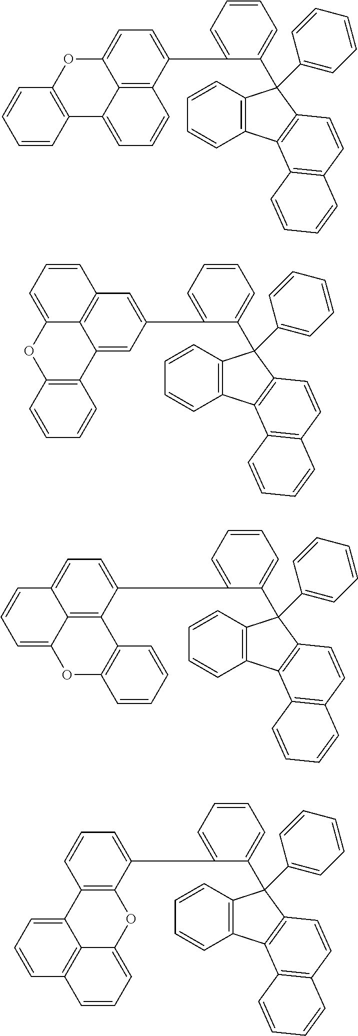

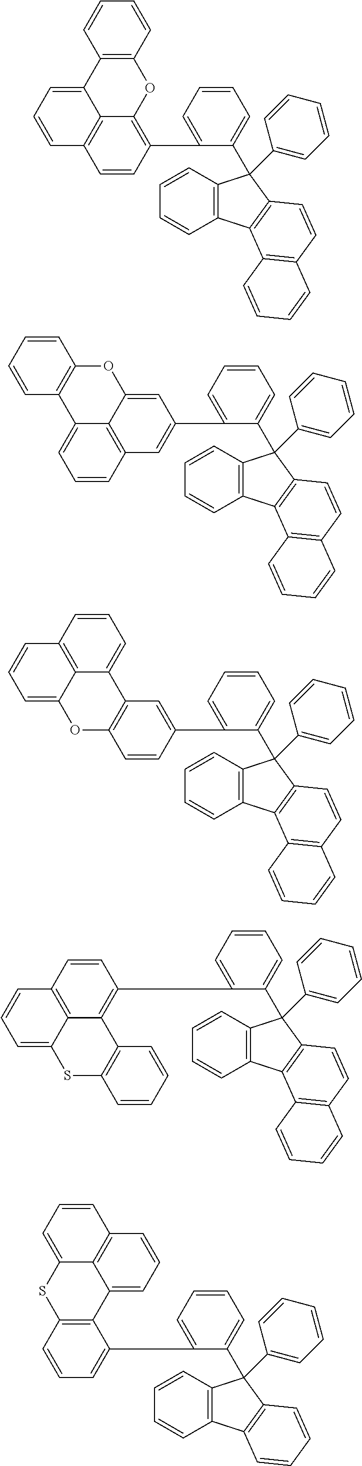

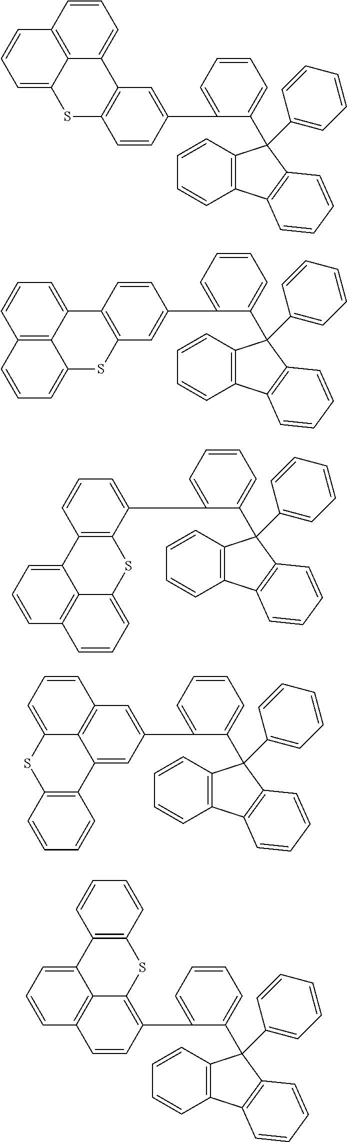

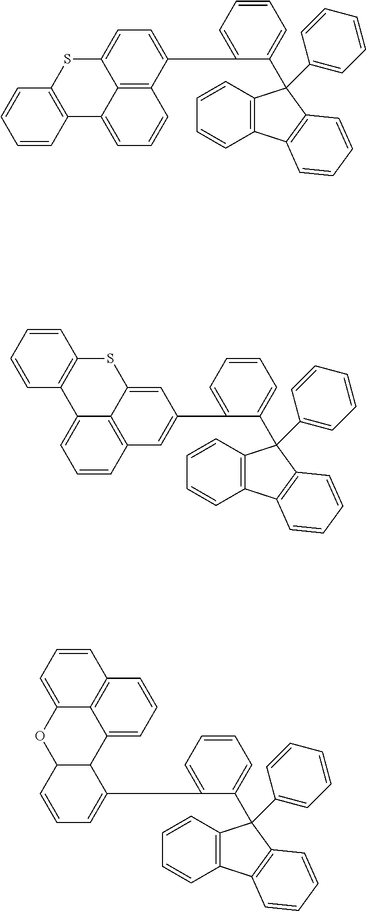 [SMILES: c1ccc(C2(c3ccccc3-c3ccc4c5c(cccc35)-c3ccccc3S4)c3ccccc3-c3ccccc32)cc1 ; c1ccc(C2(c3ccccc3-c3cc4c5c(cccc5c3)-c3ccccc3S4)c3ccccc3-c3ccccc32)cc1 ; C1=CC2Oc3cccc4cccc(c34)C2C(c2ccccc2C2(c3ccccc3)c3ccccc3-c3ccccc32)=C1]

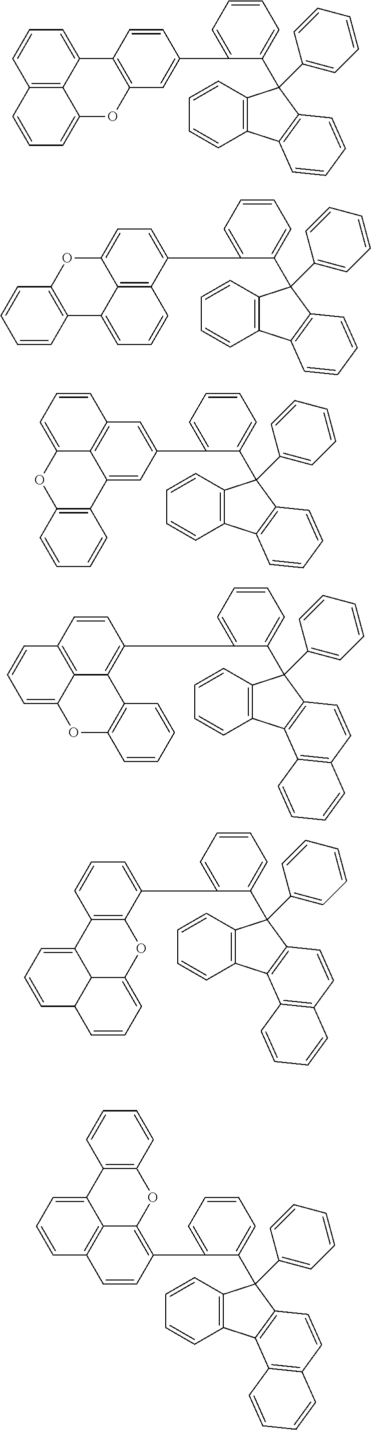

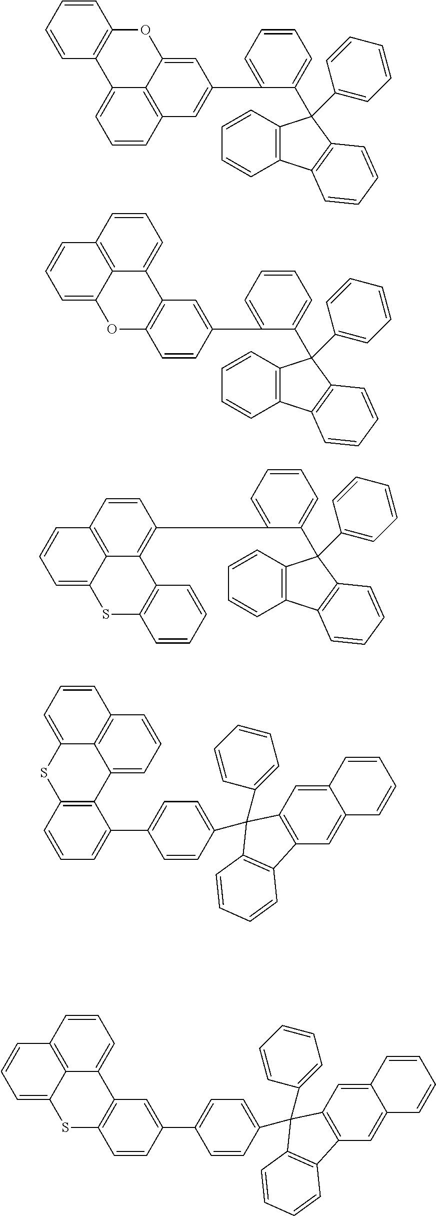

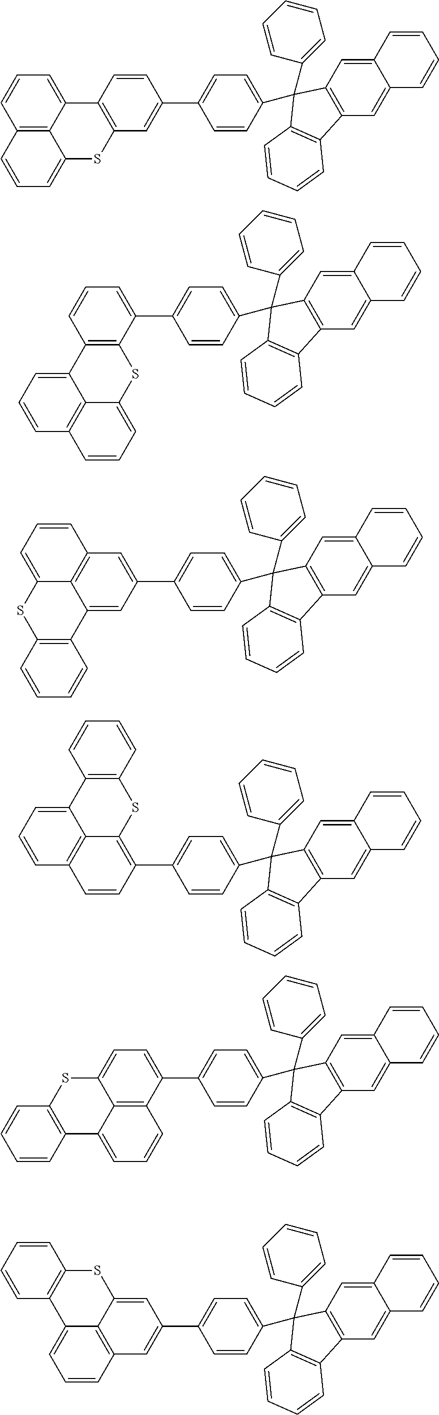

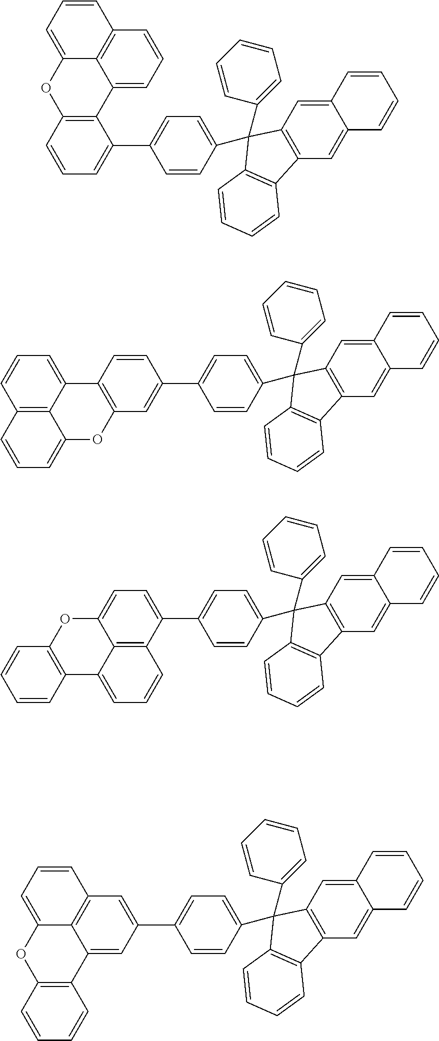 [SMILES: c1ccc(C2(c3ccc(-c4cccc5c4-c4cccc6cccc(c46)O5)cc3)c3ccccc3-c3cc4ccccc4cc32)cc1 ; c1ccc(C2(c3ccc(-c4ccc5c(c4)Oc4cccc6cccc-5c46)cc3)c3ccccc3-c3cc4ccccc4cc32)cc1 ; c1ccc(C2(c3ccc(-c4ccc5c6c(cccc46)-c4ccccc4O5)cc3)c3ccccc3-c3cc4ccccc4cc32)cc1 ; c1ccc(C2(c3ccc(-c4cc5c6c(cccc6c4)Oc4ccccc4-5)cc3)c3ccccc3-c3cc4ccccc4cc32)cc1]

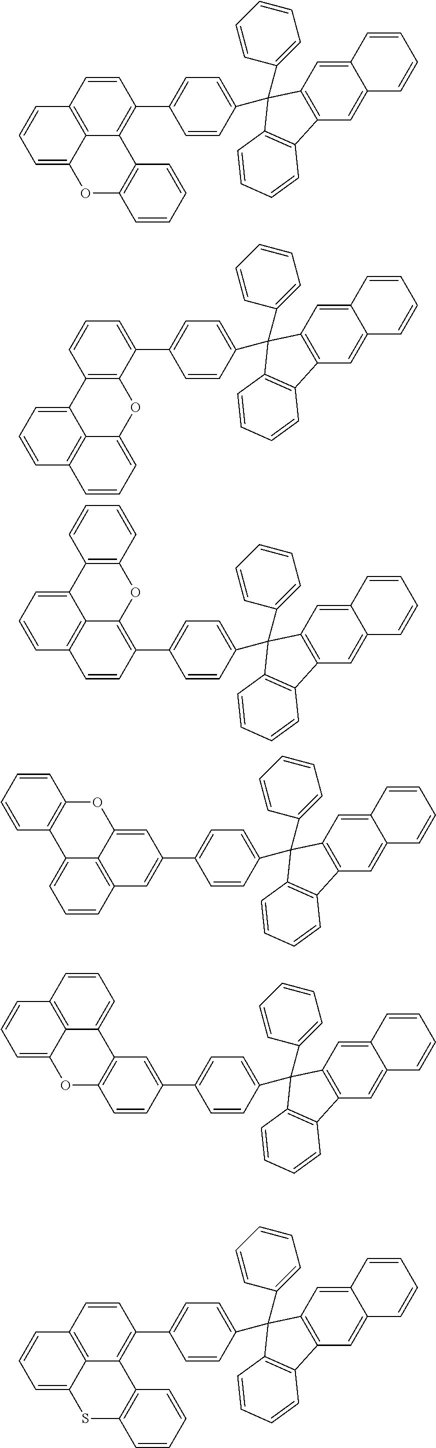

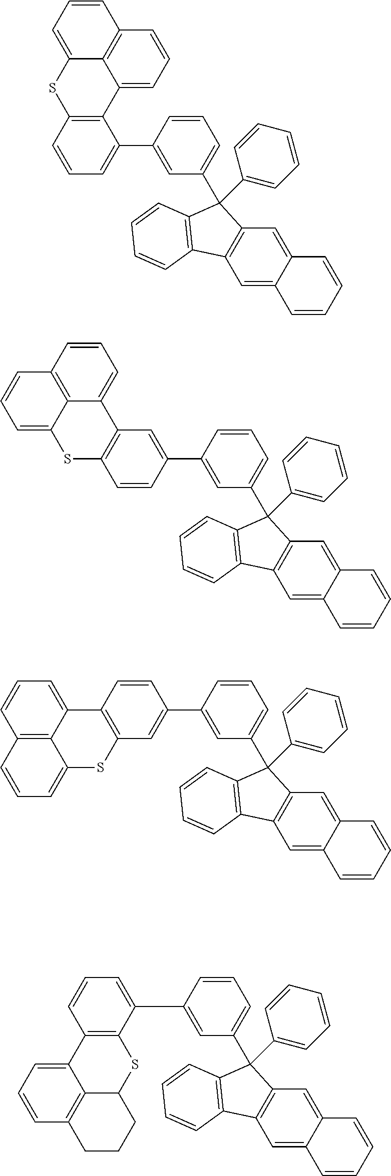 [SMILES: c1ccc(C2(c3cccc(-c4cccc5c4-c4cccc6cccc(c46)S5)c3)c3ccccc3-c3cc4ccccc4cc32)cc1 ; c1ccc(C2(c3cccc(-c4ccc5c(c4)-c4cccc6cccc(c46)S5)c3)c3ccccc3-c3cc4ccccc4cc32)cc1 ; c1ccc(C2(c3cccc(-c4ccc5c(c4)Sc4cccc6cccc-5c46)c3)c3ccccc3-c3cc4ccccc4cc32)cc1 ; c1ccc(C2(c3cccc(-c4cccc5c4SC4CCCc6cccc-5c64)c3)c3ccccc3-c3cc4ccccc4cc32)cc1]

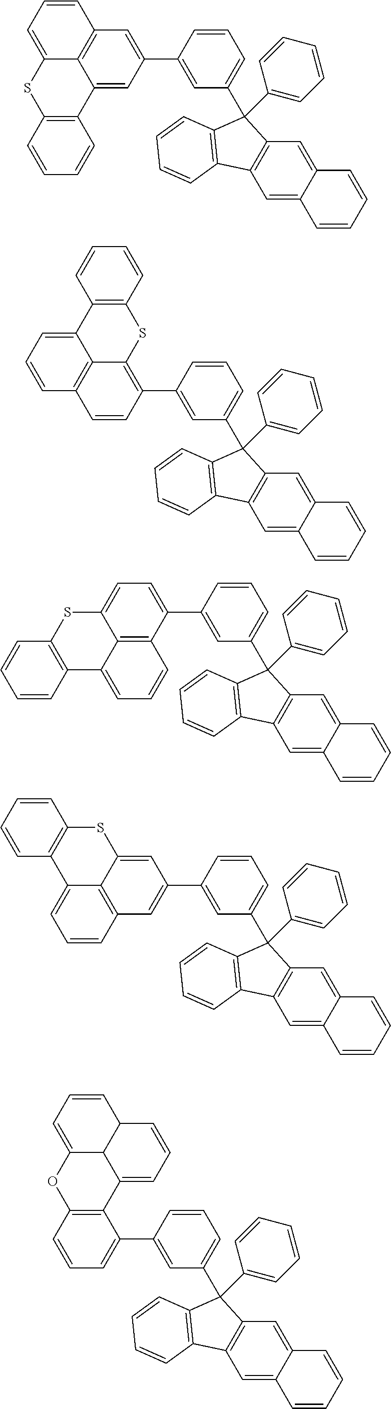

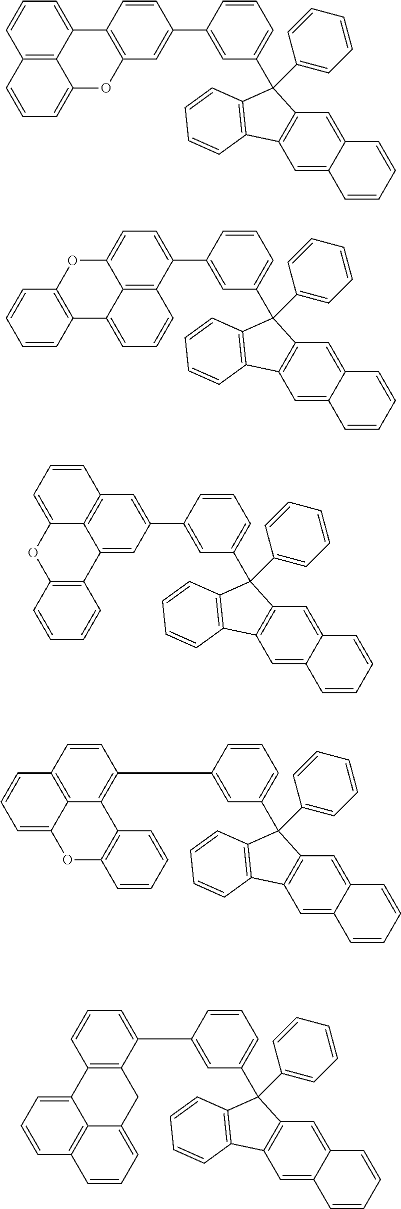

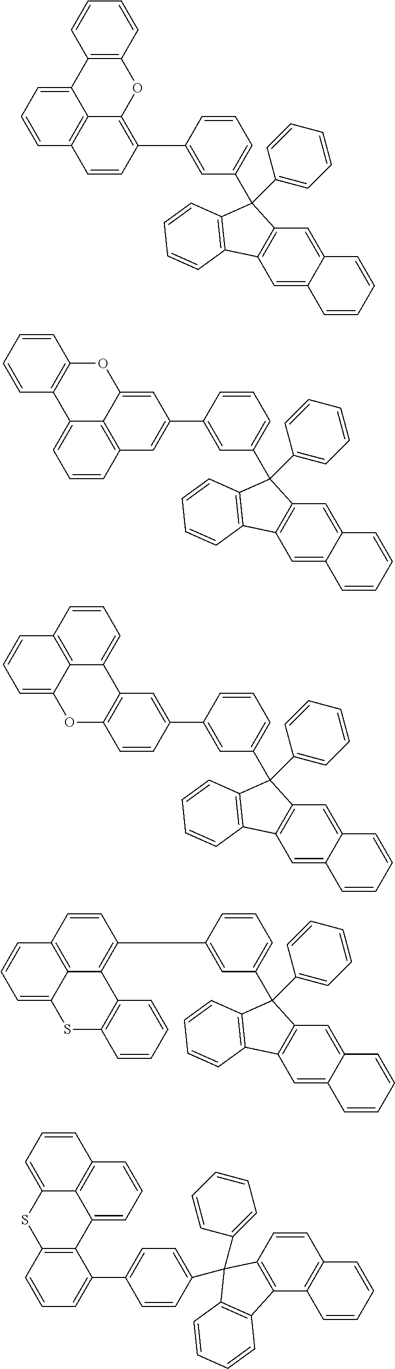

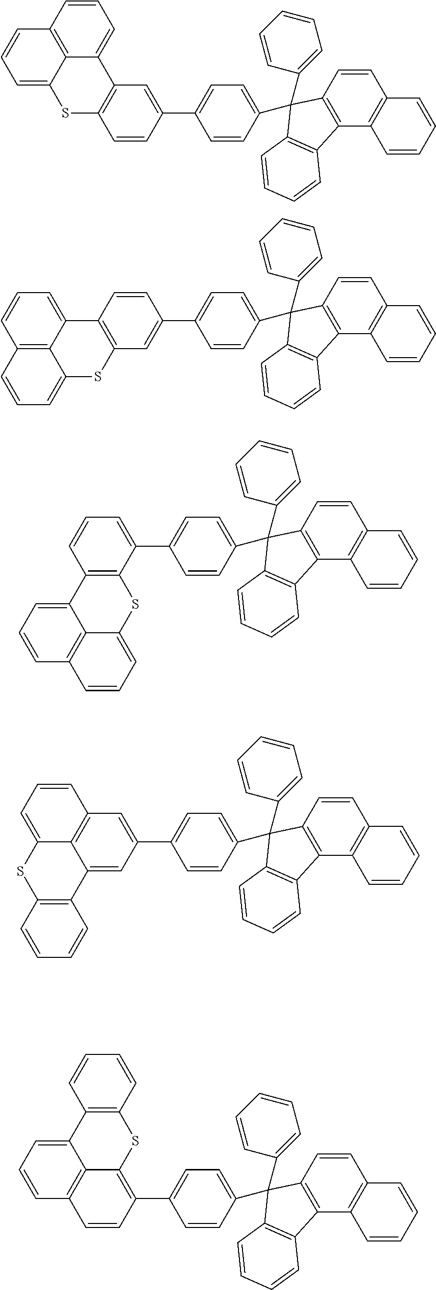

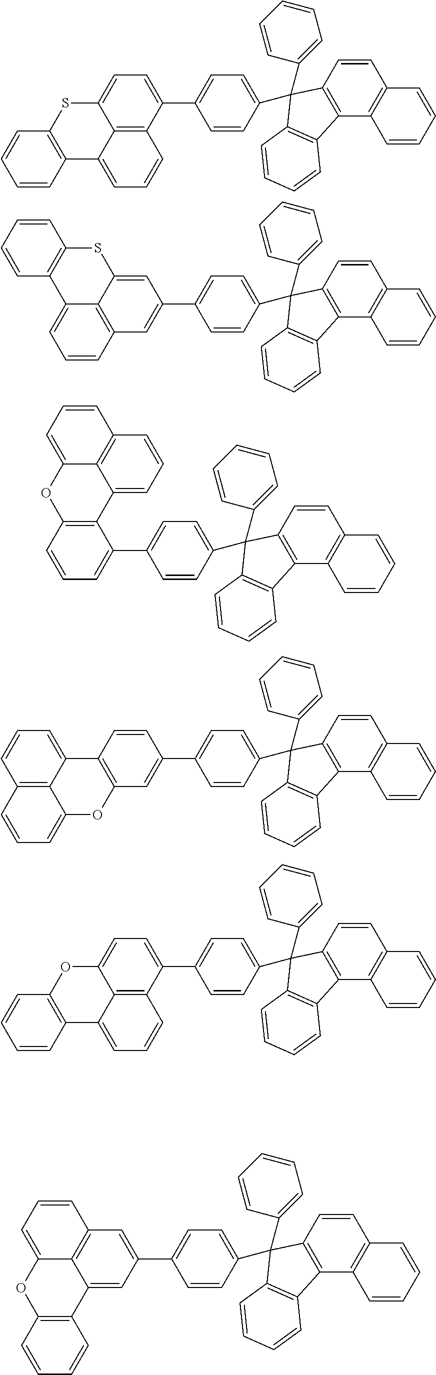

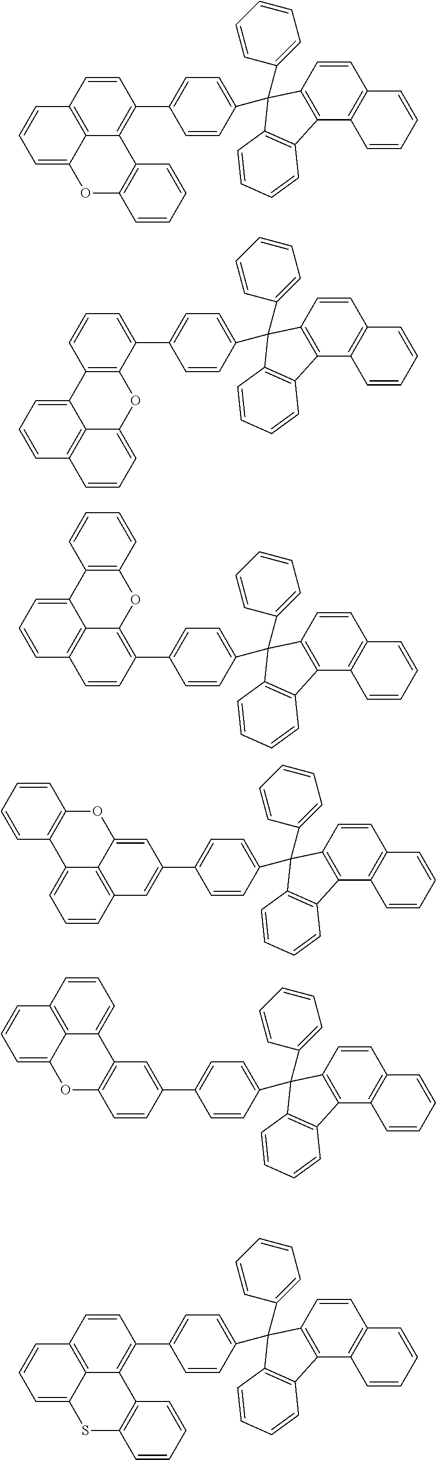

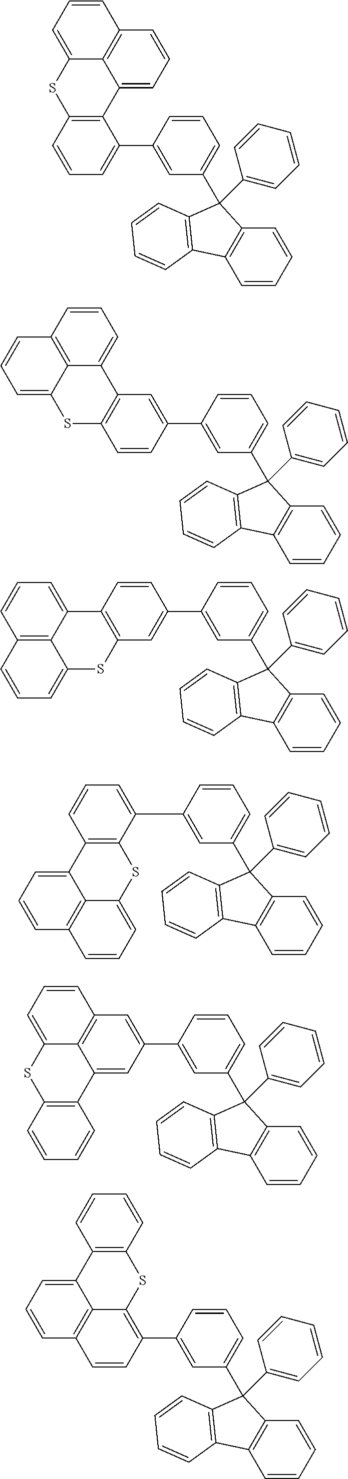

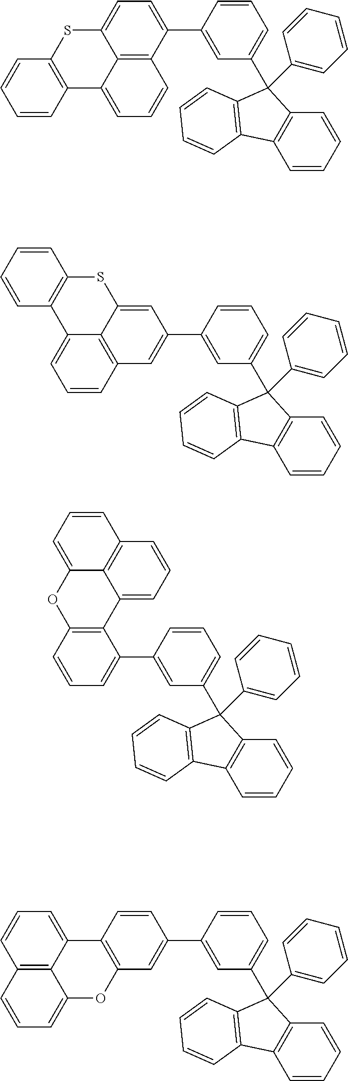 [SMILES: c1ccc(C2(c3cccc(-c4ccc5c6c(cccc46)-c4ccccc4S5)c3)c3ccccc3-c3ccccc32)cc1 ; c1ccc(C2(c3cccc(-c4cc5c6c(cccc6c4)-c4ccccc4S5)c3)c3ccccc3-c3ccccc32)cc1 ; c1ccc(C2(c3cccc(-c4cccc5c4-c4cccc6cccc(c46)O5)c3)c3ccccc3-c3ccccc32)cc1 ; c1ccc(C2(c3cccc(-c4ccc5c(c4)Oc4cccc6cccc-5c46)c3)c3ccccc3-c3ccccc32)cc1]

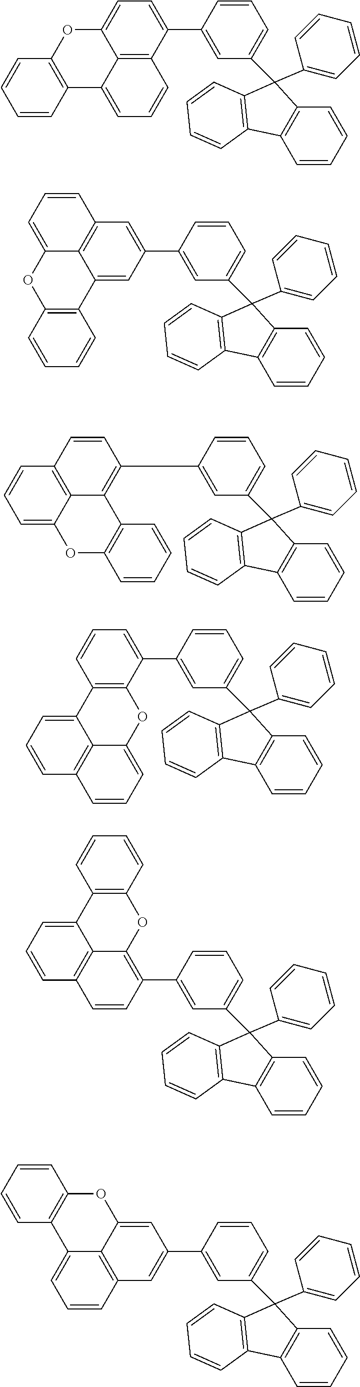

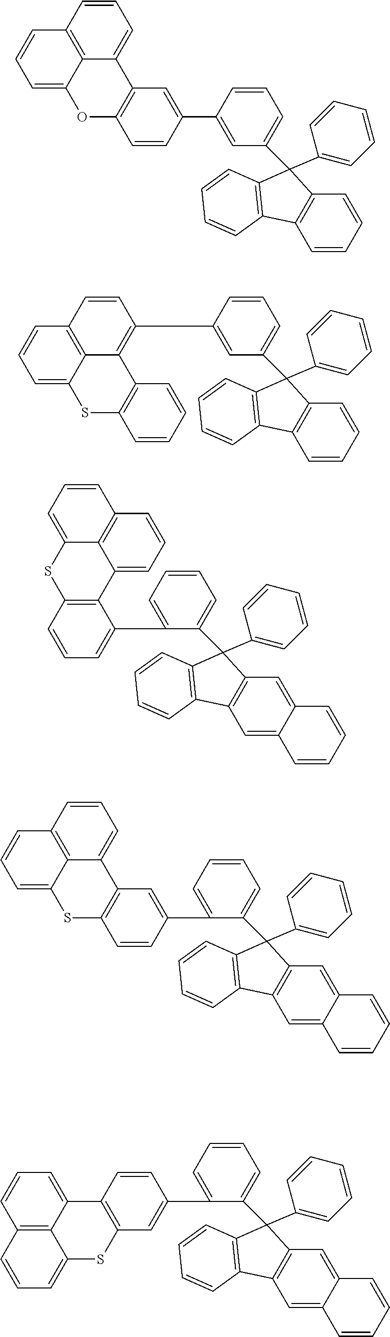 [SMILES: c1ccc(C2(c3cccc(-c4ccc5c(c4)-c4cccc6cccc(c46)O5)c3)c3ccccc3-c3ccccc32)cc1 ; c1ccc(C2(c3cccc(-c4ccc5cccc6c5c4-c4ccccc4S6)c3)c3ccccc3-c3ccccc32)cc1 ; c1ccc(C2(c3ccccc3-c3cccc4c3-c3cccc5cccc(c35)S4)c3ccccc3-c3cc4ccccc4cc32)cc1 ; c1ccc(C2(c3ccccc3-c3ccc4c(c3)-c3cccc5cccc(c35)S4)c3ccccc3-c3cc4ccccc4cc32)cc1 ; c1ccc(C2(c3ccccc3-c3ccc4c(c3)Sc3cccc5cccc-4c35)c3ccccc3-c3cc4ccccc4cc32)cc1]

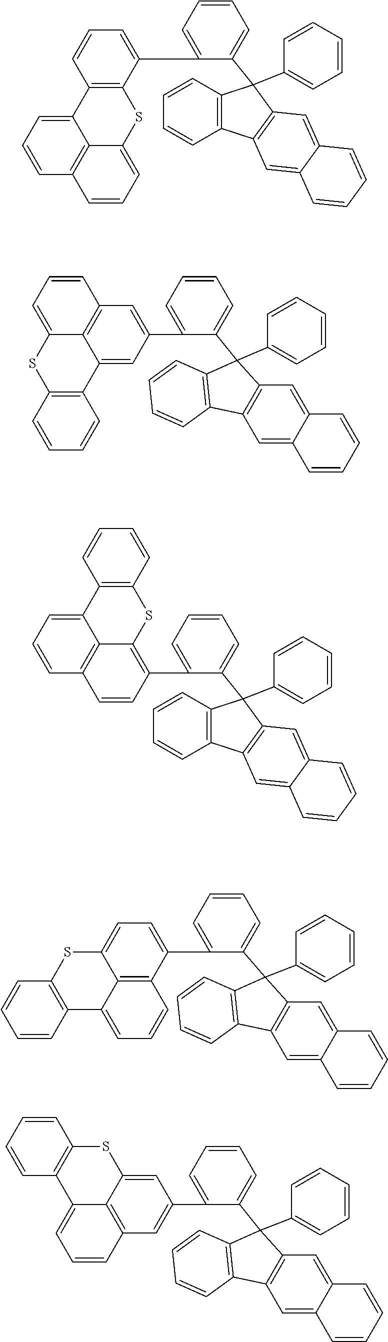 [SMILES: c1ccc(C2(c3ccccc3-c3cccc4c3Sc3cccc5cccc-4c35)c3ccccc3-c3cc4ccccc4cc32)cc1 ; c1ccc(C2(c3ccccc3-c3cc4c5c(cccc5c3)Sc3ccccc3-4)c3ccccc3-c3cc4ccccc4cc32)cc1 ; c1ccc(C2(c3ccccc3-c3ccc4cccc5c4c3Sc3ccccc3-5)c3ccccc3-c3cc4ccccc4cc32)cc1 ; c1ccc(C2(c3ccccc3-c3ccc4c5c(cccc35)-c3ccccc3S4)c3ccccc3-c3cc4ccccc4cc32)cc1 ; c1ccc(C2(c3ccccc3-c3cc4c5c(cccc5c3)-c3ccccc3S4)c3ccccc3-c3cc4ccccc4cc32)cc1]

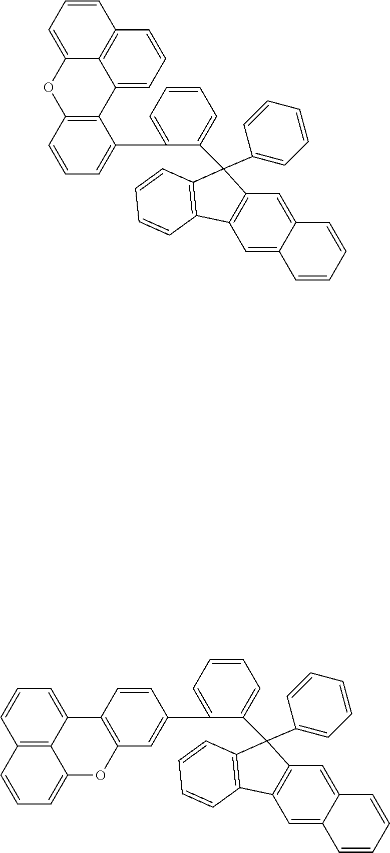 [SMILES: c1ccc(C2(c3ccccc3-c3cccc4c3-c3cccc5cccc(c35)O4)c3ccccc3-c3cc4ccccc4cc32)cc1 ; c1ccc(C2(c3ccccc3-c3ccc4c(c3)Oc3cccc5cccc-4c35)c3ccccc3-c3cc4ccccc4cc32)cc1]

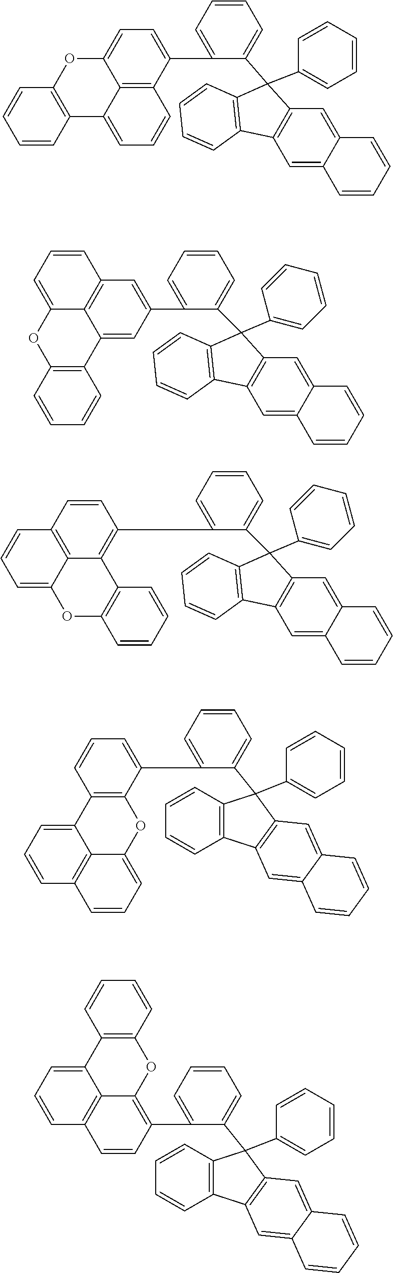

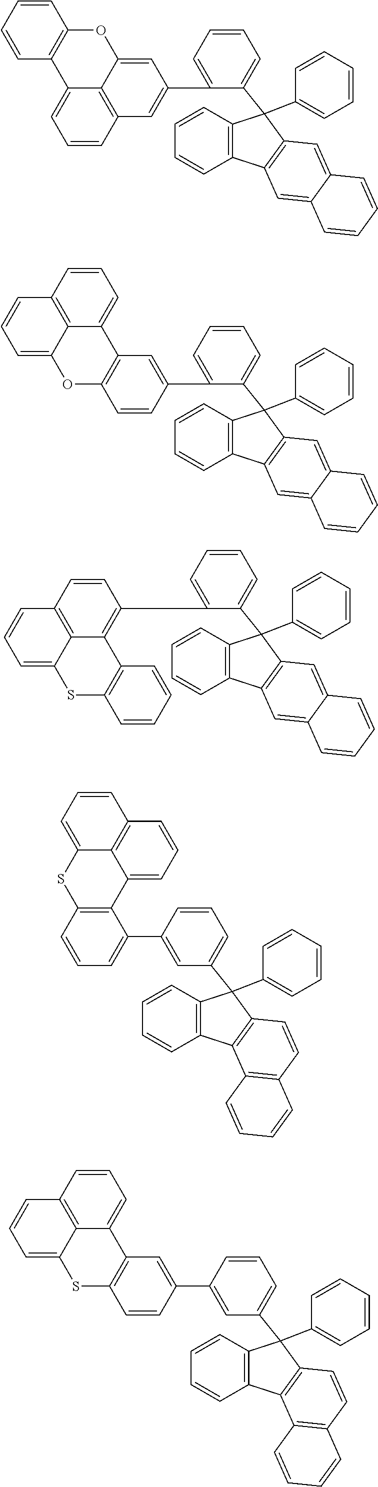

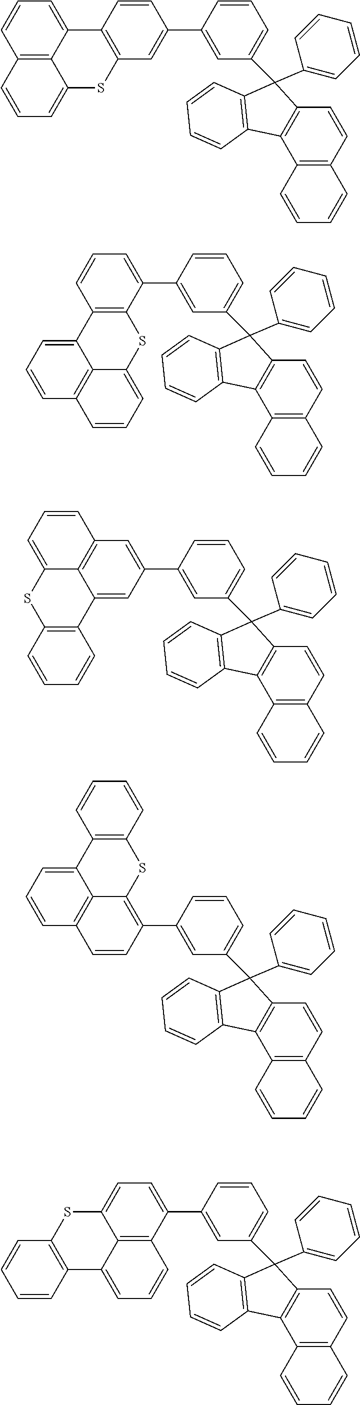

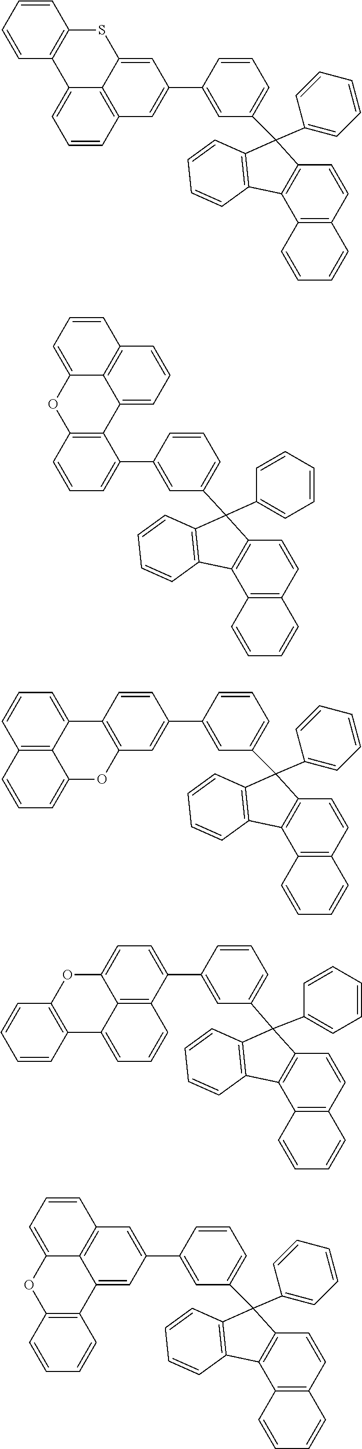

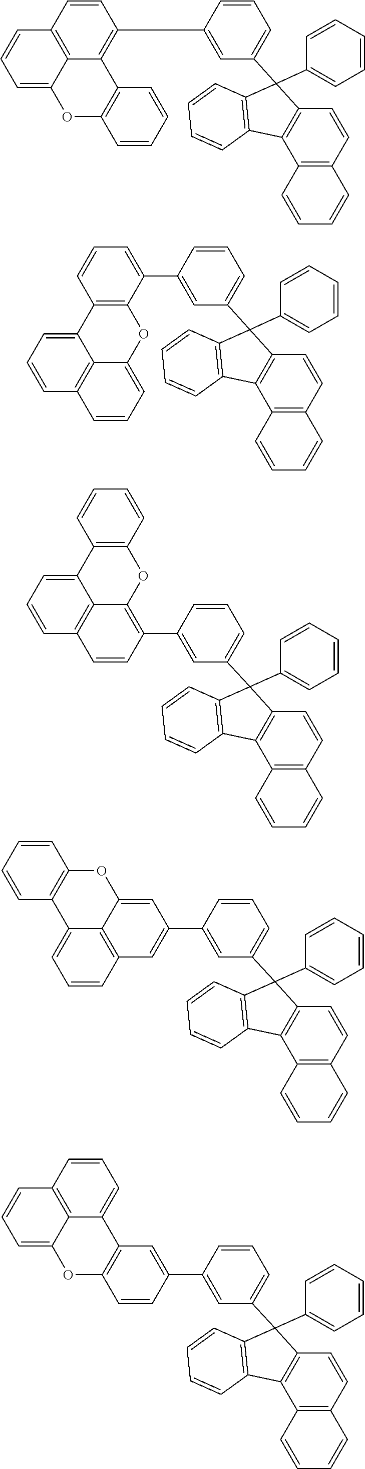

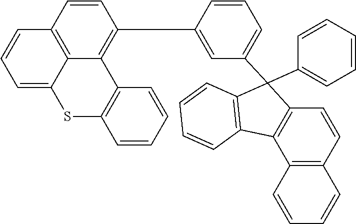 [SMILES: c1ccc(C2(c3cccc(-c4ccc5cccc6c5c4-c4ccccc4S6)c3)c3ccccc3-c3c2ccc2ccccc32)cc1]

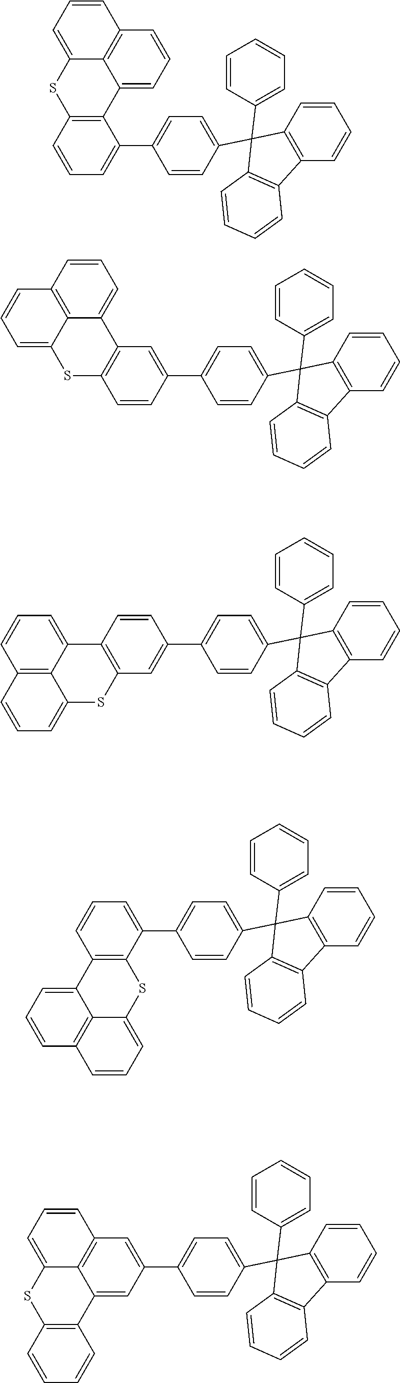 [SMILES: c1ccc(C2(c3ccc(-c4cccc5c4-c4cccc6cccc(c46)S5)cc3)c3ccccc3-c3ccccc32)cc1 ; c1ccc(C2(c3ccc(-c4ccc5c(c4)-c4cccc6cccc(c46)S5)cc3)c3ccccc3-c3ccccc32)cc1 ; c1ccc(C2(c3ccc(-c4ccc5c(c4)Sc4cccc6cccc-5c46)cc3)c3ccccc3-c3ccccc32)cc1 ; c1ccc(C2(c3ccc(-c4cccc5c4Sc4cccc6cccc-5c46)cc3)c3ccccc3-c3ccccc32)cc1 ; c1ccc(C2(c3ccc(-c4cc5c6c(cccc6c4)Sc4ccccc4-5)cc3)c3ccccc3-c3ccccc32)cc1]

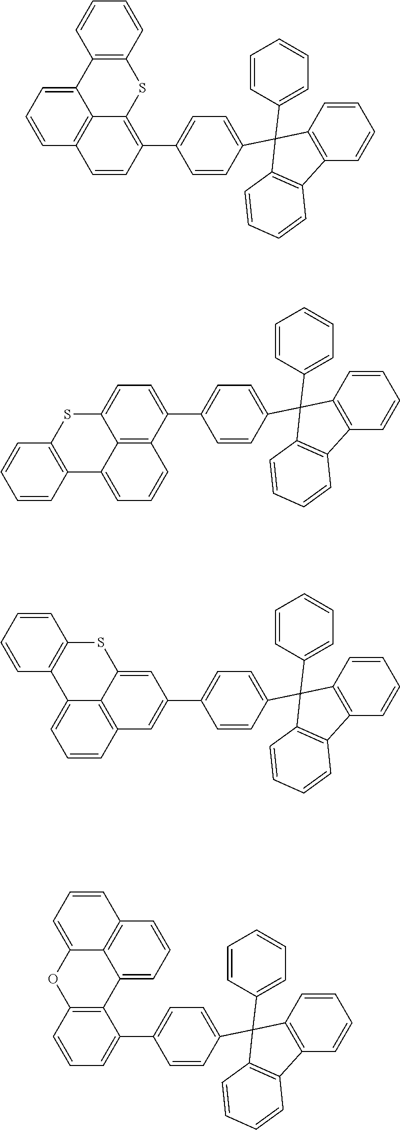 [SMILES: c1ccc(C2(c3ccc(-c4ccc5cccc6c5c4Sc4ccccc4-6)cc3)c3ccccc3-c3ccccc32)cc1 ; c1ccc(C2(c3ccc(-c4ccc5c6c(cccc46)-c4ccccc4S5)cc3)c3ccccc3-c3ccccc32)cc1 ; c1ccc(C2(c3ccc(-c4cc5c6c(cccc6c4)-c4ccccc4S5)cc3)c3ccccc3-c3ccccc32)cc1 ; c1ccc(C2(c3ccc(-c4cccc5c4-c4cccc6cccc(c46)O5)cc3)c3ccccc3-c3ccccc32)cc1]

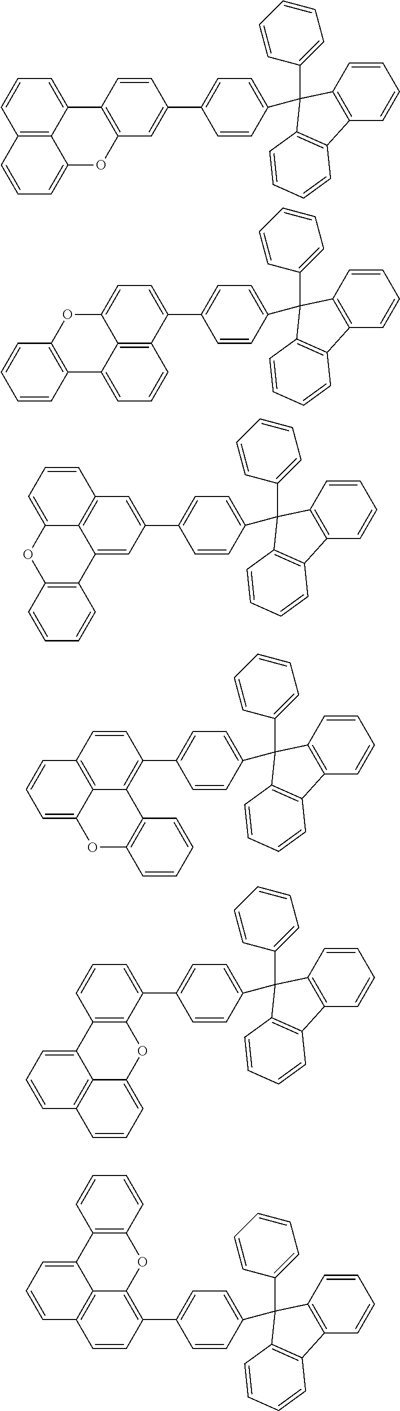

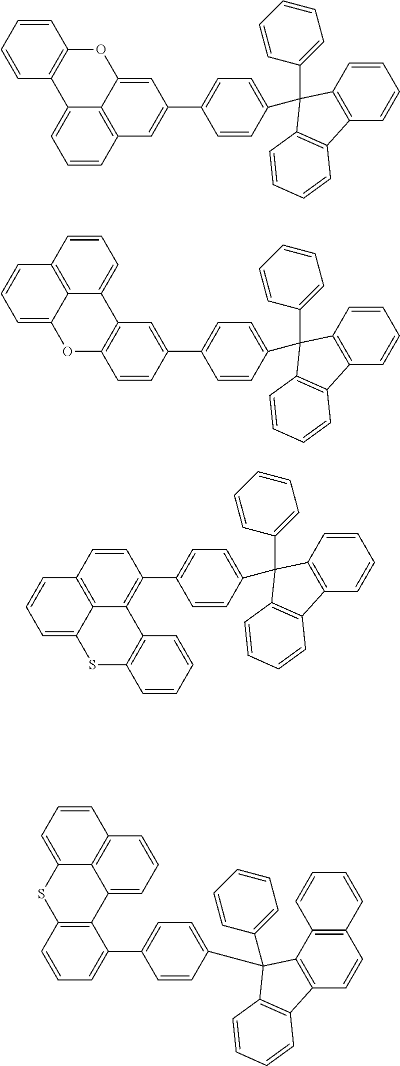

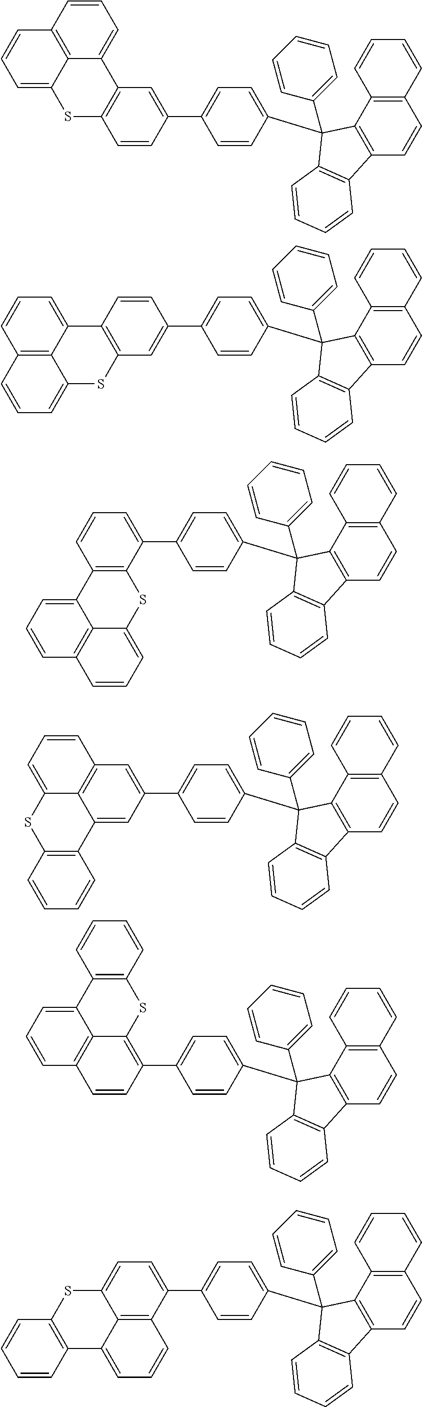

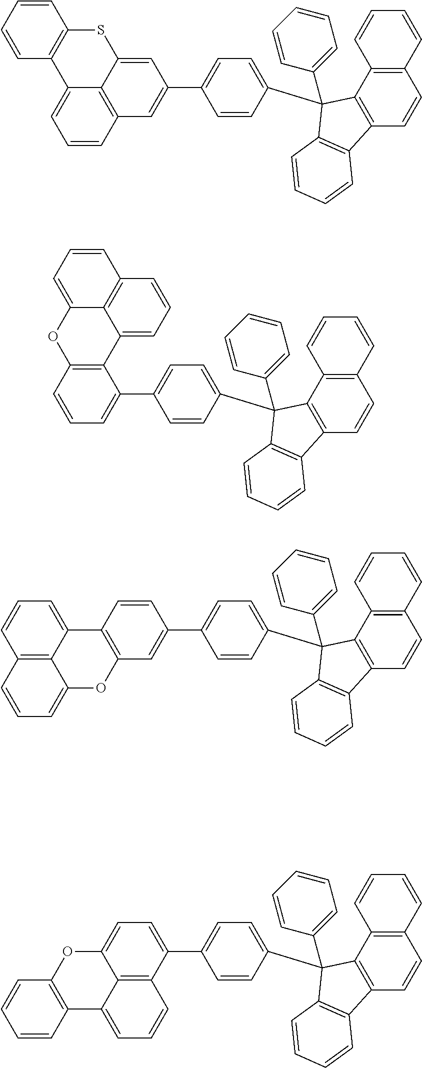

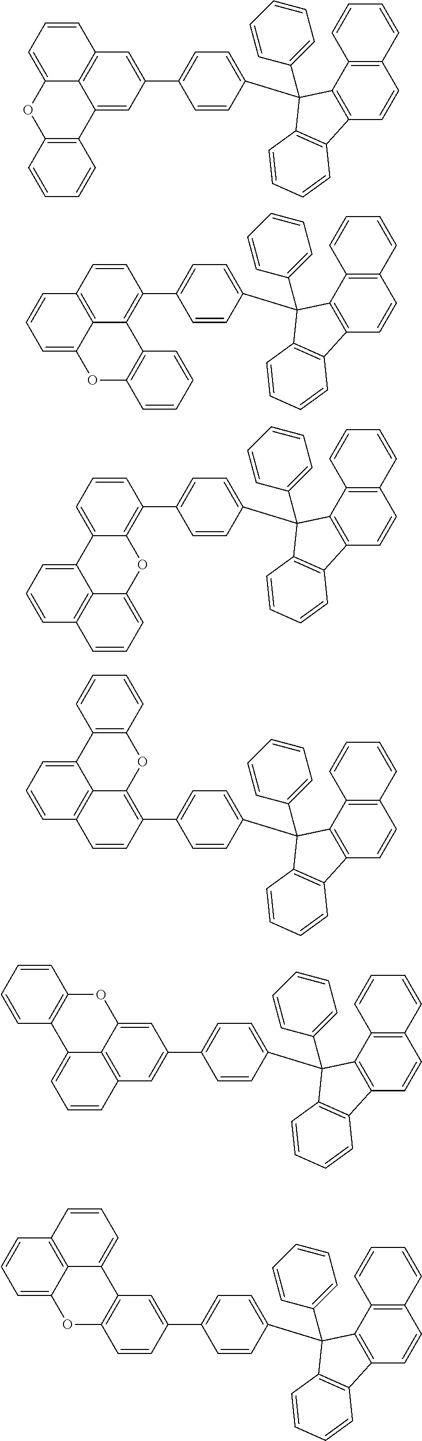

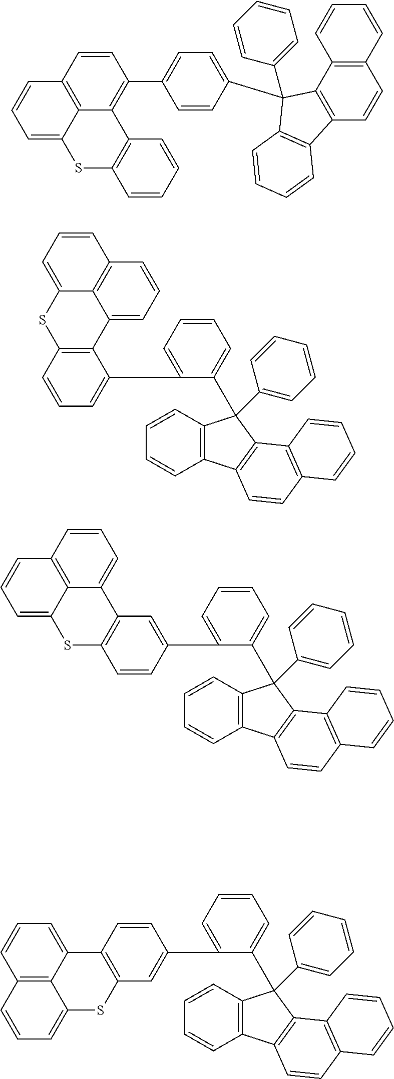 [SMILES: c1ccc(C2(c3ccc(-c4ccc5cccc6c5c4-c4ccccc4S6)cc3)c3ccccc3-c3ccc4ccccc4c32)cc1 ; c1ccc(C2(c3ccccc3-c3cccc4c3-c3cccc5cccc(c35)S4)c3ccccc3-c3ccc4ccccc4c32)cc1 ; c1ccc(C2(c3ccccc3-c3ccc4c(c3)-c3cccc5cccc(c35)S4)c3ccccc3-c3ccc4ccccc4c32)cc1 ; c1ccc(C2(c3ccccc3-c3ccc4c(c3)Sc3cccc5cccc-4c35)c3ccccc3-c3ccc4ccccc4c32)cc1]

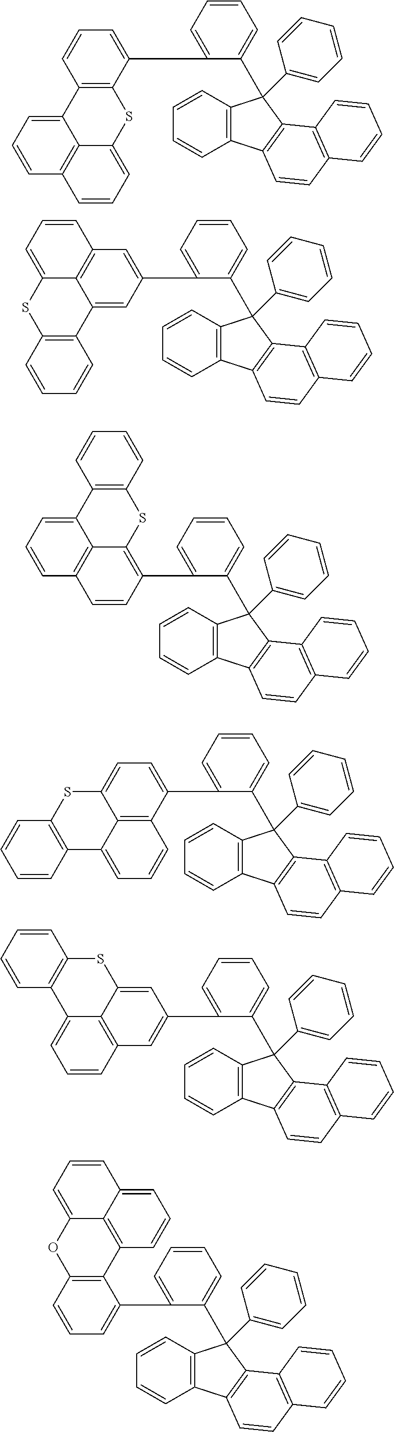

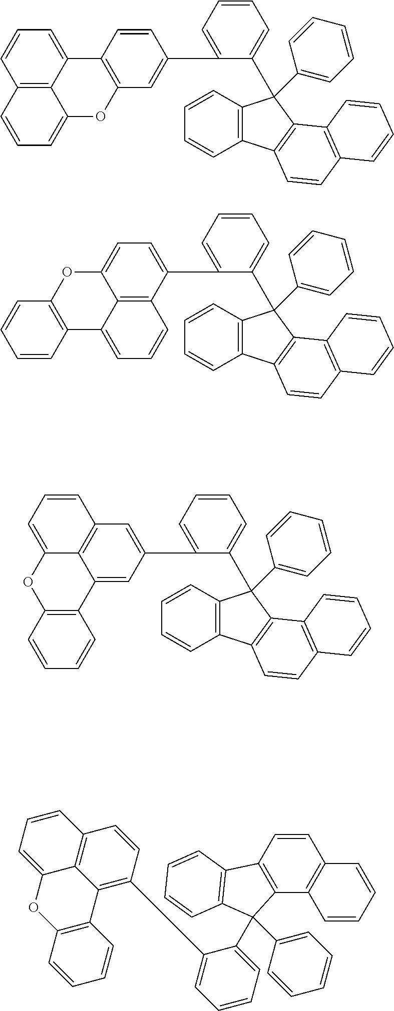 [SMILES: c1ccc(C2(c3ccccc3-c3ccc4c(c3)Oc3cccc5cccc-4c35)c3ccccc3-c3ccc4ccccc4c32)cc1 ; c1ccc(C2(c3ccccc3-c3ccc4c5c(cccc35)-c3ccccc3O4)c3ccccc3-c3ccc4ccccc4c32)cc1 ; c1ccc(C2(c3ccccc3-c3cc4c5c(cccc5c3)Oc3ccccc3-4)c3ccccc3-c3ccc4ccccc4c32)cc1 ; c1ccc(C2(c3ccccc3-c3ccc4cccc5c4c3-c3ccccc3O5)c3ccccc3-c3ccc4ccccc4c32)cc1]

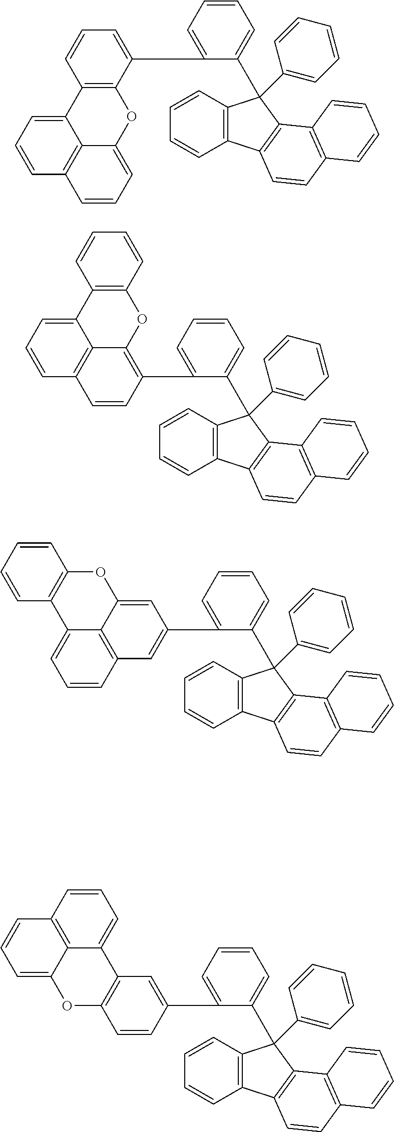

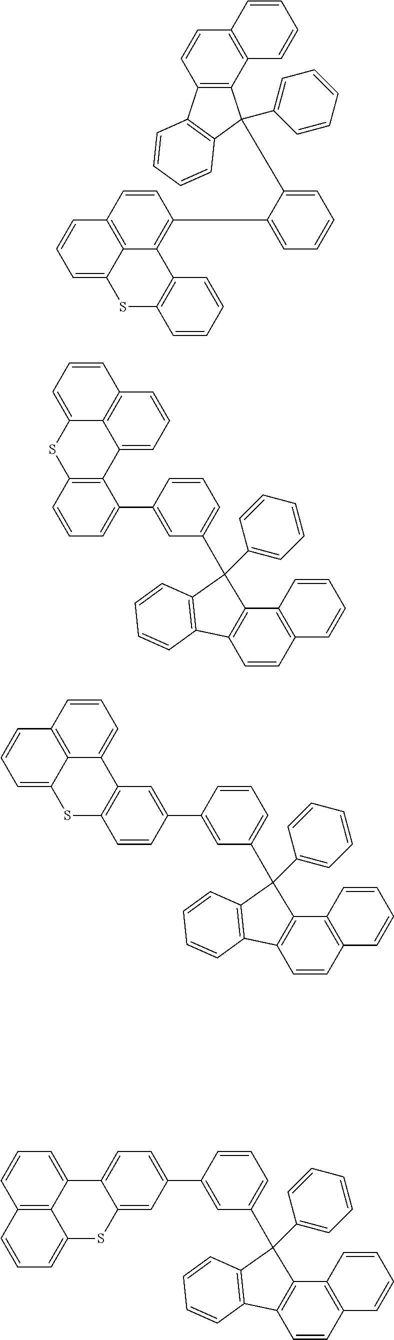 [SMILES: c1ccc(C2(c3ccccc3-c3ccc4cccc5c4c3-c3ccccc3S5)c3ccccc3-c3ccc4ccccc4c32)cc1 ; c1ccc(C2(c3cccc(-c4cccc5c4-c4cccc6cccc(c46)S5)c3)c3ccccc3-c3ccc4ccccc4c32)cc1 ; c1ccc(C2(c3cccc(-c4ccc5c(c4)-c4cccc6cccc(c46)S5)c3)c3ccccc3-c3ccc4ccccc4c32)cc1 ; c1ccc(C2(c3cccc(-c4ccc5c(c4)Sc4cccc6cccc-5c46)c3)c3ccccc3-c3ccc4ccccc4c32)cc1]

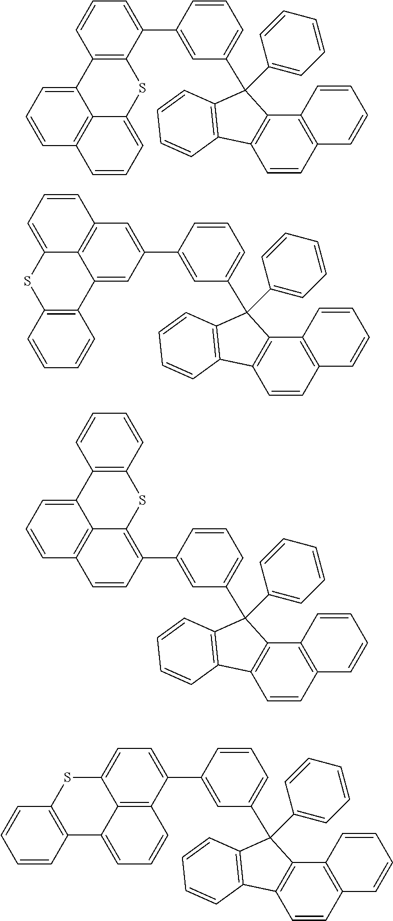

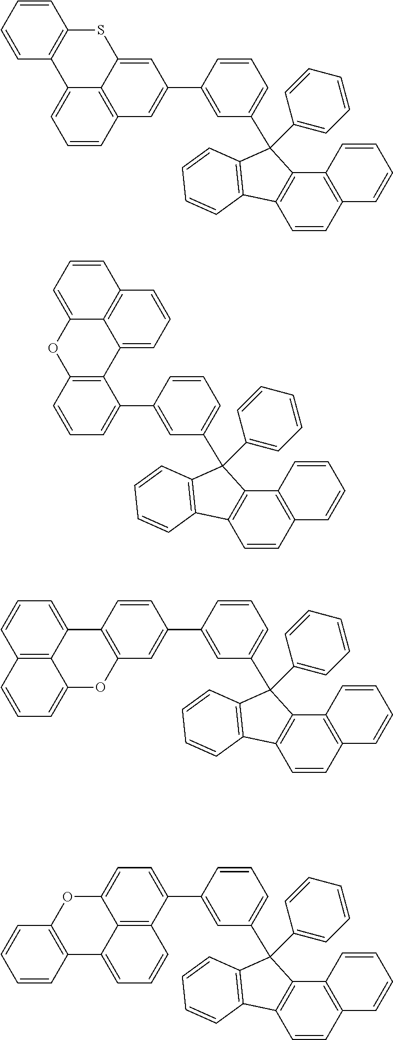 [SMILES: c1ccc(C2(c3cccc(-c4cc5c6c(cccc6c4)-c4ccccc4S5)c3)c3ccccc3-c3ccc4ccccc4c32)cc1 ; c1ccc(C2(c3cccc(-c4cccc5c4-c4cccc6cccc(c46)O5)c3)c3ccccc3-c3ccc4ccccc4c32)cc1 ; c1ccc(C2(c3cccc(-c4ccc5c(c4)Oc4cccc6cccc-5c46)c3)c3ccccc3-c3ccc4ccccc4c32)cc1 ; c1ccc(C2(c3cccc(-c4ccc5c6c(cccc46)-c4ccccc4O5)c3)c3ccccc3-c3ccc4ccccc4c32)cc1]

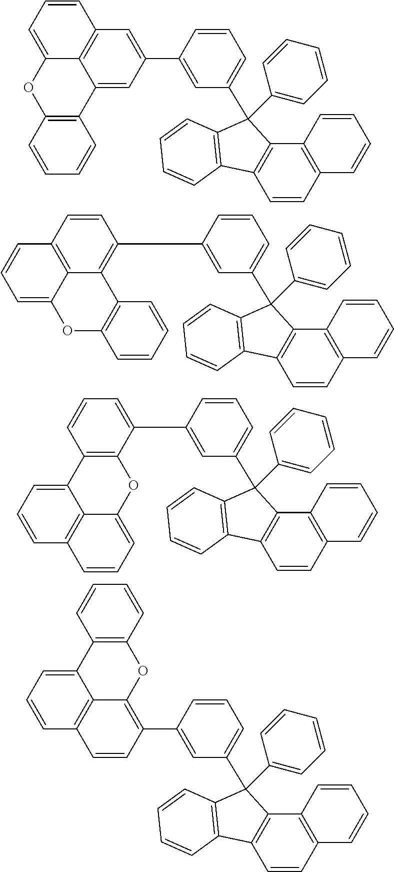

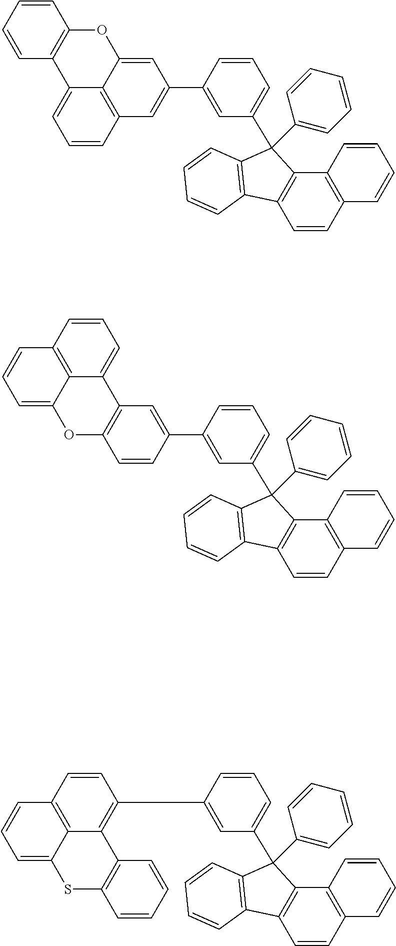 [SMILES: c1ccc(C2(c3cccc(-c4cc5c6c(cccc6c4)-c4ccccc4O5)c3)c3ccccc3-c3ccc4ccccc4c32)cc1 ; c1ccc(C2(c3cccc(-c4ccc5c(c4)-c4cccc6cccc(c46)O5)c3)c3ccccc3-c3ccc4ccccc4c32)cc1 ; c1ccc(C2(c3cccc(-c4ccc5cccc6c5c4-c4ccccc4S6)c3)c3ccccc3-c3ccc4ccccc4c32)cc1]

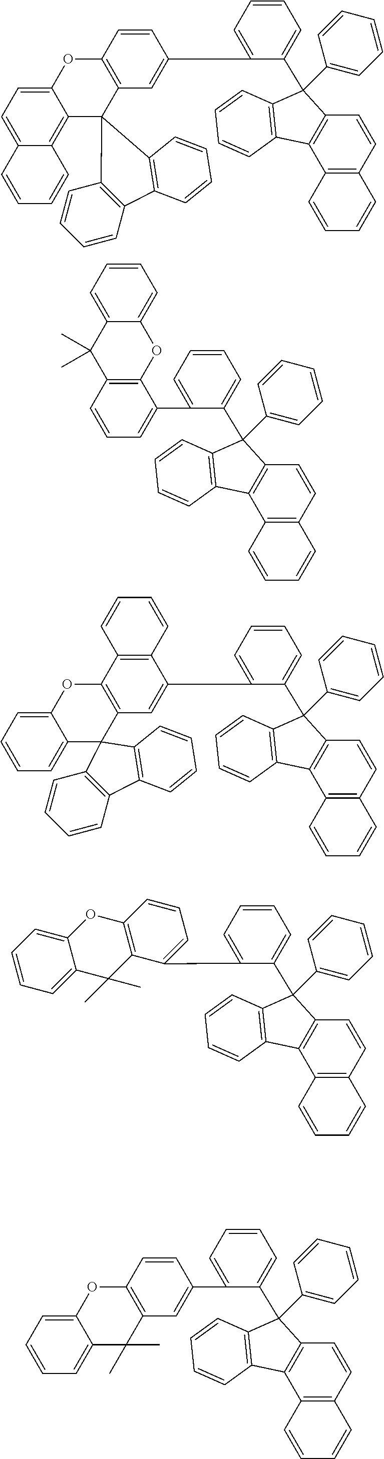

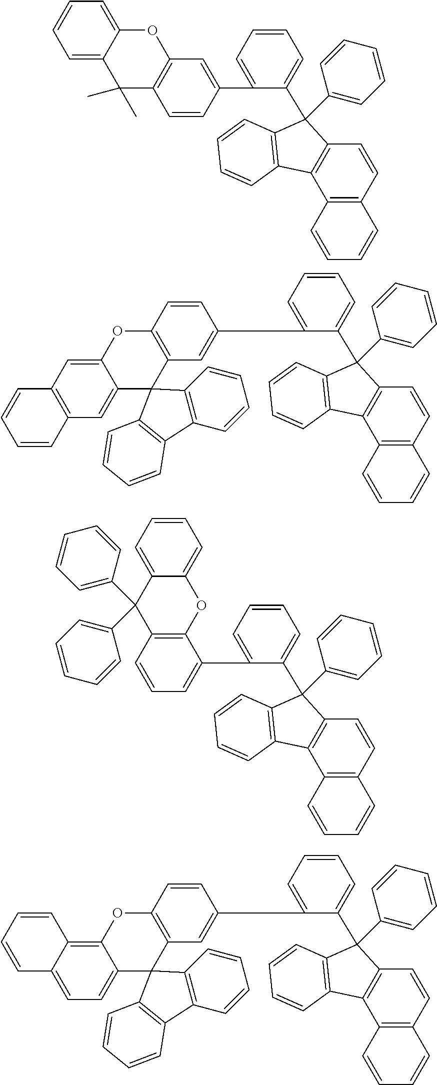

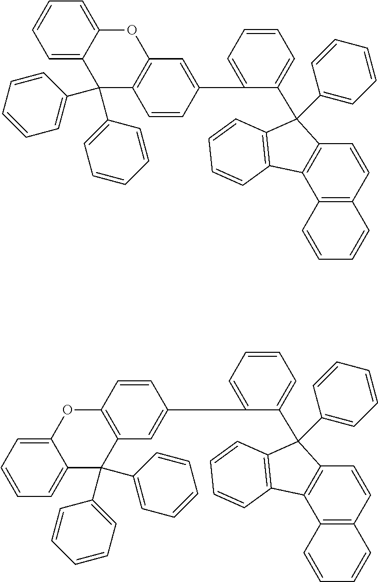 [SMILES: c1ccc(C2(c3ccccc3)c3ccccc3Oc3cc(-c4ccccc4C4(c5ccccc5)c5ccccc5-c5c4ccc4ccccc54)ccc32)cc1 ; c1ccc(C2(c3ccccc3)c3ccccc3Oc3ccc(-c4ccccc4C4(c5ccccc5)c5ccccc5-c5c4ccc4ccccc54)cc32)cc1]

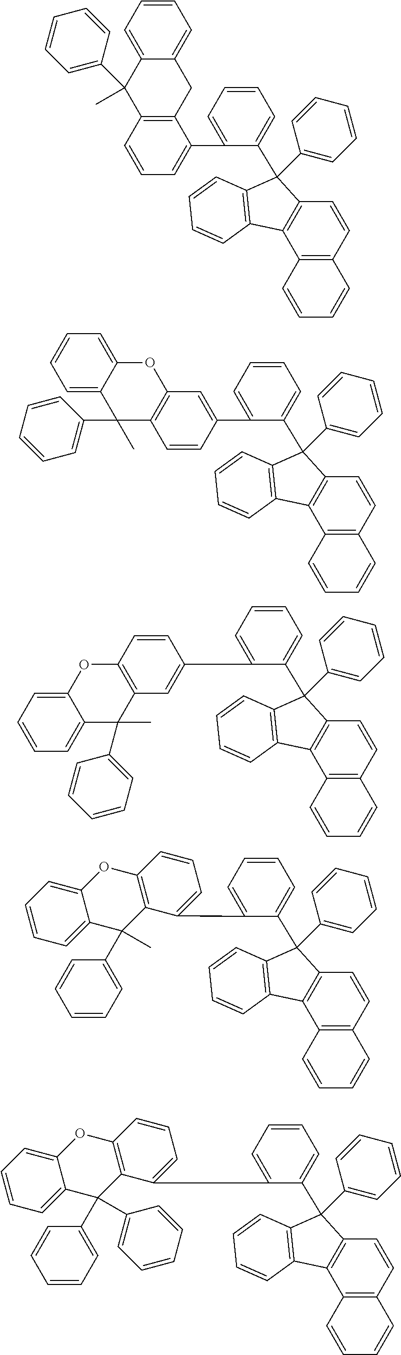

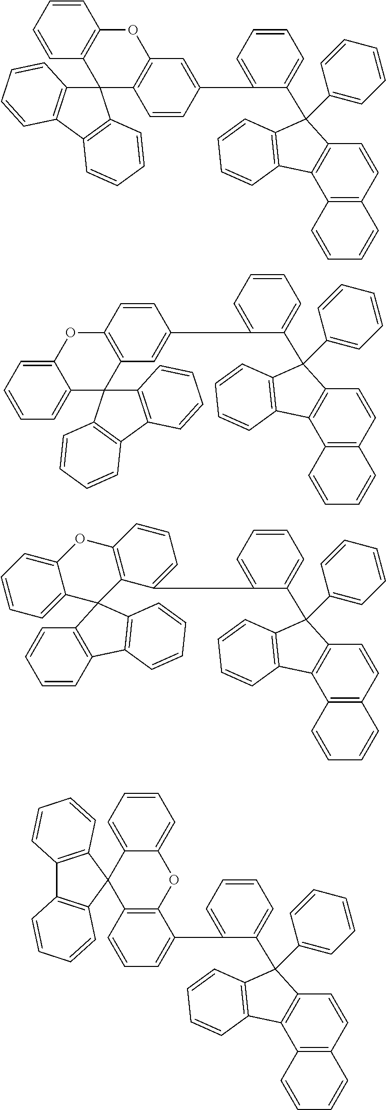

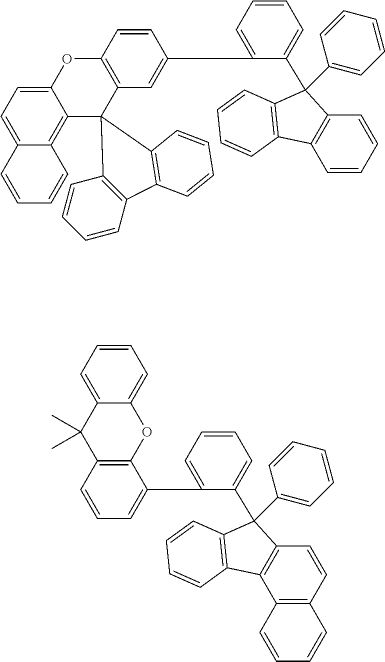 [SMILES: c1ccc(C2(c3ccccc3-c3ccc4c(c3)C3(c5ccccc5-c5ccccc53)c3c(ccc5ccccc35)O4)c3ccccc3-c3ccccc32)cc1 ; CC1(C)c2ccccc2Oc2c(-c3ccccc3C3(c4ccccc4)c4ccccc4-c4c3ccc3ccccc43)cccc21]

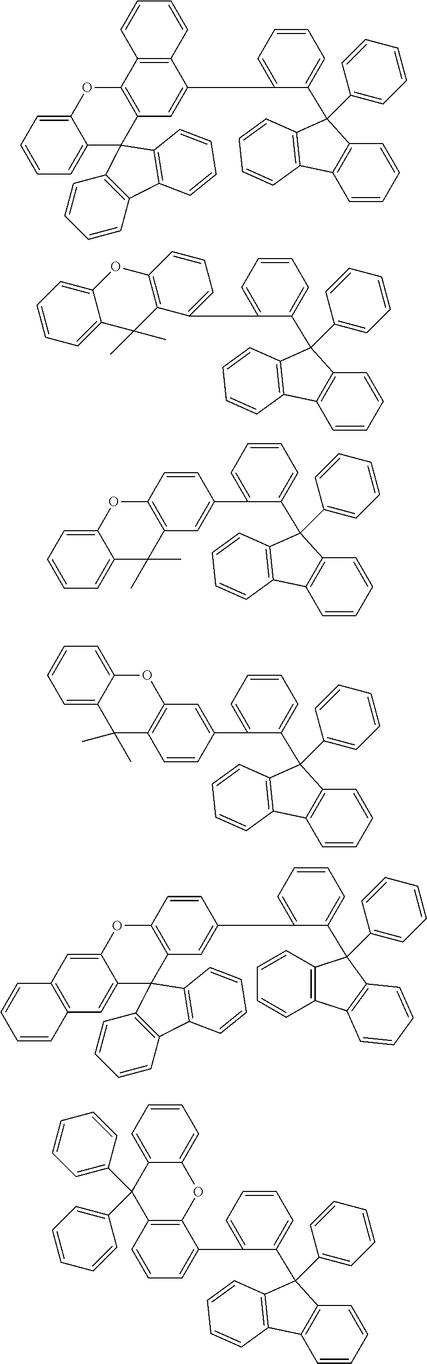

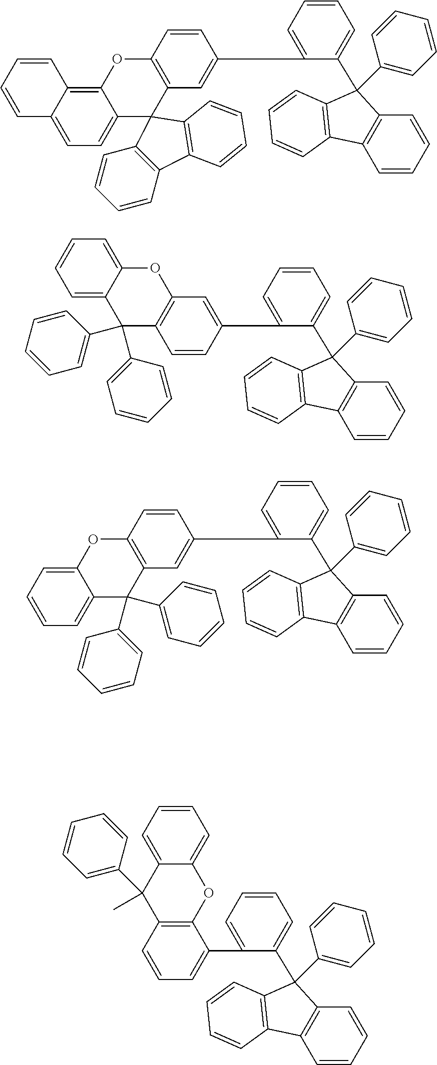

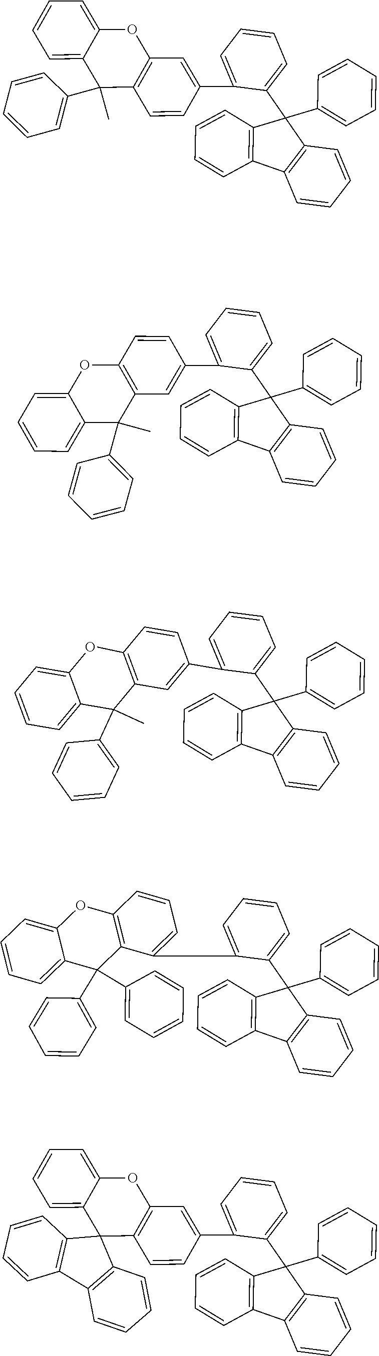 [SMILES: CC1(c2ccccc2)c2ccccc2Oc2cc(-c3ccccc3C3(c4ccccc4)c4ccccc4-c4ccccc43)ccc21 ; CC1(c2ccccc2)c2ccccc2Oc2ccc(-c3ccccc3C3(c4ccccc4)c4ccccc4-c4ccccc43)cc21 ; CC1(c2ccccc2)c2ccccc2Oc2ccc(-c3ccccc3C3(c4ccccc4)c4ccccc4-c4ccccc43)cc21 ; c1ccc(C2(c3ccccc3-c3cccc4c3C(c3ccccc3)(c3ccccc3)c3ccccc3O4)c3ccccc3-c3ccccc32)cc1 ; c1ccc(C2(c3ccccc3-c3ccc4c(c3)Oc3ccccc3C43c4ccccc4-c4ccccc43)c3ccccc3-c3ccccc32)cc1]

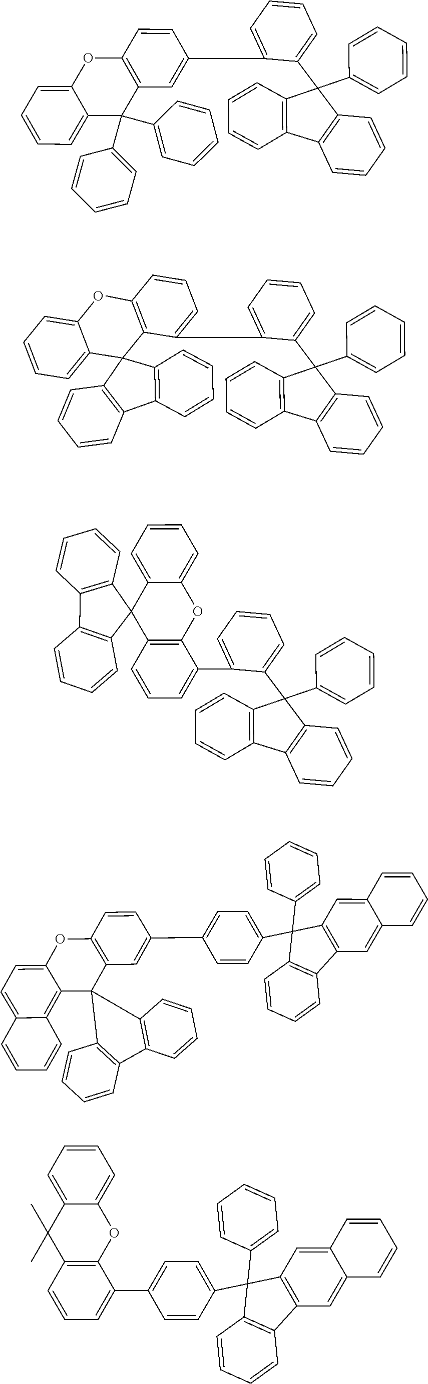

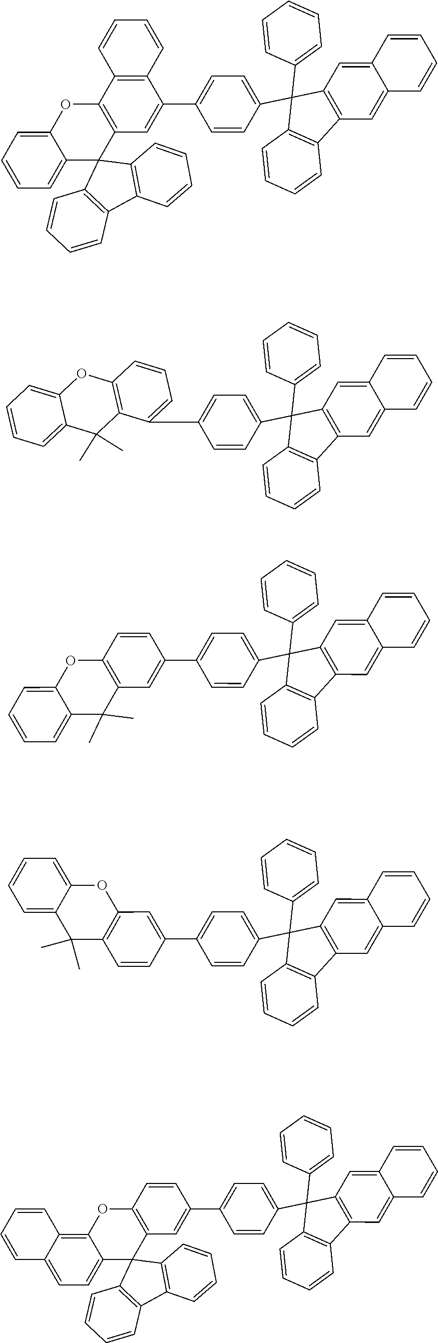 [SMILES: c1ccc(C2(c3ccc(-c4cc5c(c6ccccc46)Oc4ccccc4C54c5ccccc5-c5ccccc54)cc3)c3ccccc3-c3cc4ccccc4cc32)cc1 ; CC1(C)c2ccccc2Oc2cccc(-c3ccc(C4(c5ccccc5)c5ccccc5-c5cc6ccccc6cc54)cc3)c21 ; CC1(C)c2ccccc2Oc2ccc(-c3ccc(C4(c5ccccc5)c5ccccc5-c5cc6ccccc6cc54)cc3)cc21 ; CC1(C)c2ccccc2Oc2cc(-c3ccc(C4(c5ccccc5)c5ccccc5-c5cc6ccccc6cc54)cc3)ccc21 ; c1ccc(C2(c3ccc(-c4ccc5c(c4)C4(c6ccccc6-c6ccccc64)c4ccc6ccccc6c4O5)cc3)c3ccccc3-c3cc4ccccc4cc32)cc1]

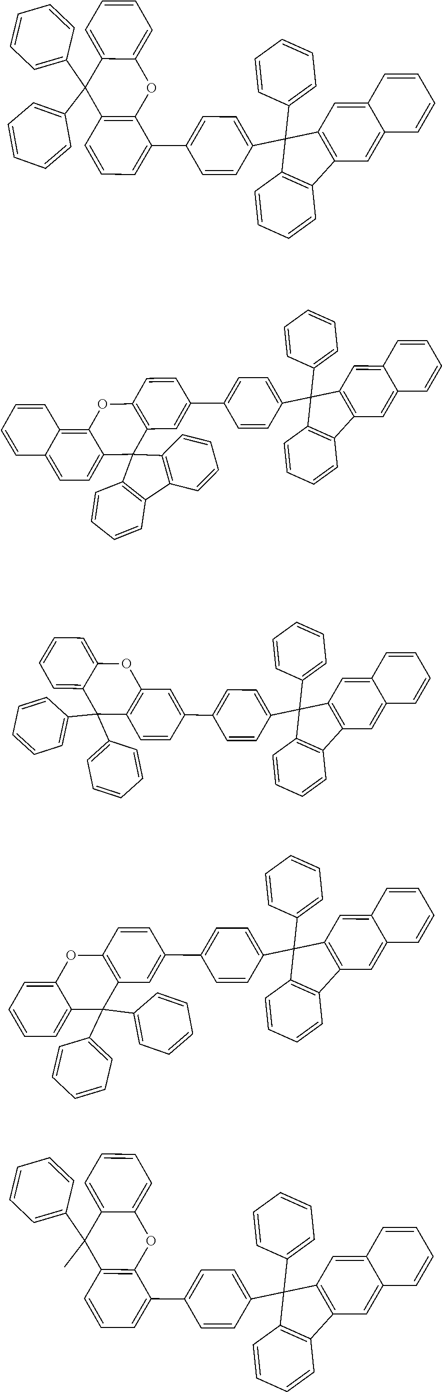 [SMILES: c1ccc(C2(c3ccc(-c4cccc5c4Oc4ccccc4C5(c4ccccc4)c4ccccc4)cc3)c3ccccc3-c3cc4ccccc4cc32)cc1 ; c1ccc(C2(c3ccc(-c4ccc5c(c4)C4(c6ccccc6-c6ccccc64)c4ccc6ccccc6c4O5)cc3)c3ccccc3-c3cc4ccccc4cc32)cc1 ; c1ccc(C2(c3ccccc3)c3ccccc3Oc3cc(-c4ccc(C5(c6ccccc6)c6ccccc6-c6cc7ccccc7cc65)cc4)ccc32)cc1 ; c1ccc(C2(c3ccccc3)c3ccccc3Oc3ccc(-c4ccc(C5(c6ccccc6)c6ccccc6-c6cc7ccccc7cc65)cc4)cc32)cc1 ; CC1(c2ccccc2)c2ccccc2Oc2c(-c3ccc(C4(c5ccccc5)c5ccccc5-c5cc6ccccc6cc54)cc3)cccc21]

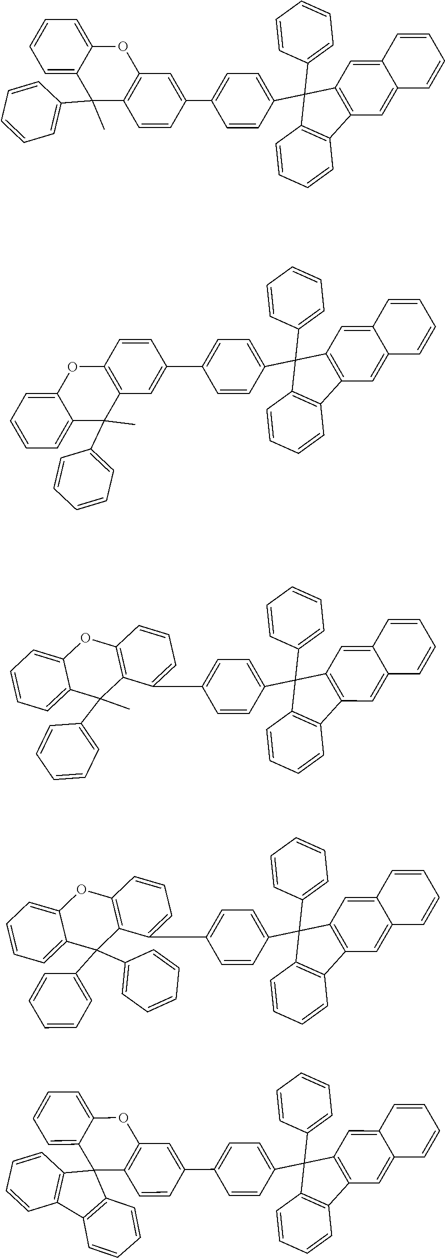 [SMILES: CC1(c2ccccc2)c2ccccc2Oc2cc(-c3ccc(C4(c5ccccc5)c5ccccc5-c5cc6ccccc6cc54)cc3)ccc21 ; CC1(c2ccccc2)c2ccccc2Oc2ccc(-c3ccc(C4(c5ccccc5)c5ccccc5-c5cc6ccccc6cc54)cc3)cc21 ; CC1(c2ccccc2)c2ccccc2Oc2cccc(-c3ccc(C4(c5ccccc5)c5ccccc5-c5cc6ccccc6cc54)cc3)c21 ; c1ccc(C2(c3ccc(-c4cccc5c4C(c4ccccc4)(c4ccccc4)c4ccccc4O5)cc3)c3ccccc3-c3cc4ccccc4cc32)cc1 ; c1ccc(C2(c3ccc(-c4ccc5c(c4)Oc4ccccc4C54c5ccccc5-c5ccccc54)cc3)c3ccccc3-c3cc4ccccc4cc32)cc1]

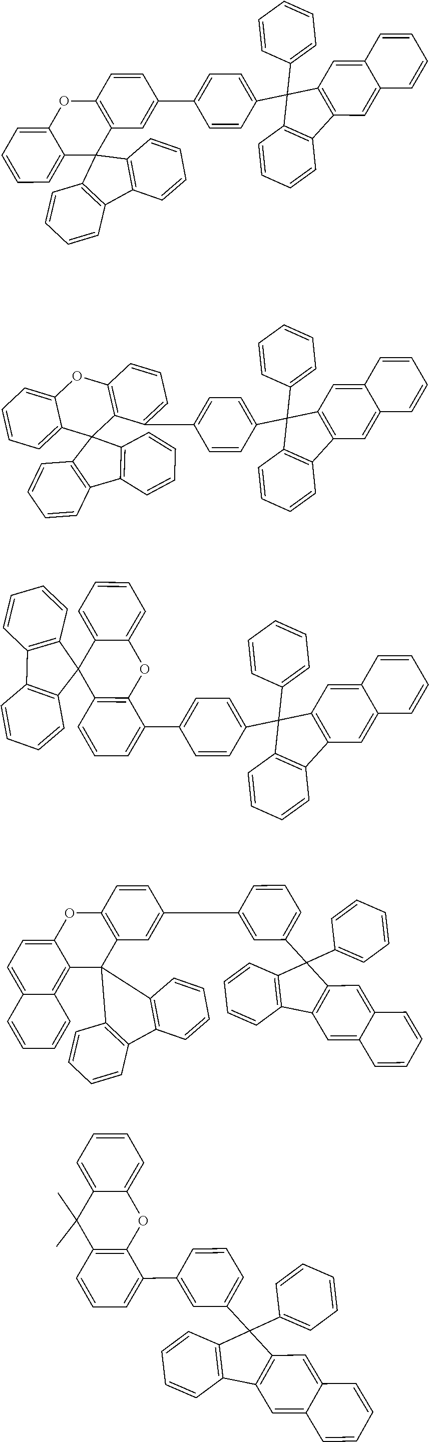 [SMILES: c1ccc(C2(c3ccc(-c4ccc5c(c4)C4(c6ccccc6O5)c5ccccc5-c5ccccc54)cc3)c3ccccc3-c3cc4ccccc4cc32)cc1 ; c1ccc(C2(c3ccc(-c4cccc5c4C4(c6ccccc6O5)c5ccccc5-c5ccccc54)cc3)c3ccccc3-c3cc4ccccc4cc32)cc1 ; c1ccc(C2(c3ccc(-c4cccc5c4Oc4ccccc4C54c5ccccc5-c5ccccc54)cc3)c3ccccc3-c3cc4ccccc4cc32)cc1 ; c1ccc(C2(c3cccc(-c4ccc5c(c4)C4(c6ccccc6-c6ccccc64)c4c(ccc6ccccc46)O5)c3)c3ccccc3-c3cc4ccccc4cc32)cc1 ; CC1(C)c2ccccc2Oc2c(-c3cccc(C4(c5ccccc5)c5ccccc5-c5cc6ccccc6cc54)c3)cccc21]

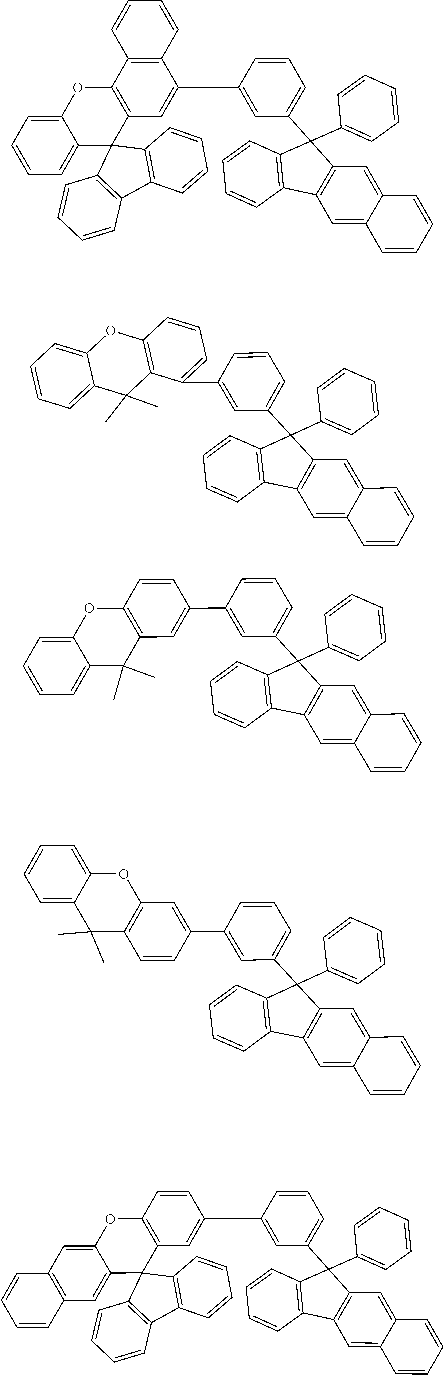 [SMILES: c1ccc(C2(c3cccc(-c4cc5c(c6ccccc46)Oc4ccccc4C54c5ccccc5-c5ccccc54)c3)c3ccccc3-c3cc4ccccc4cc32)cc1 ; CC1(C)c2ccccc2Oc2cccc(-c3cccc(C4(c5ccccc5)c5ccccc5-c5cc6ccccc6cc54)c3)c21 ; CC1(C)c2ccccc2Oc2ccc(-c3cccc(C4(c5ccccc5)c5ccccc5-c5cc6ccccc6cc54)c3)cc21 ; CC1(C)c2ccccc2Oc2cc(-c3cccc(C4(c5ccccc5)c5ccccc5-c5cc6ccccc6cc54)c3)ccc21 ; c1ccc(C2(c3cccc(-c4ccc5c(c4)C4(c6cc7ccccc7cc6O5)c5ccccc5-c5ccccc54)c3)c3ccccc3-c3cc4ccccc4cc32)cc1]

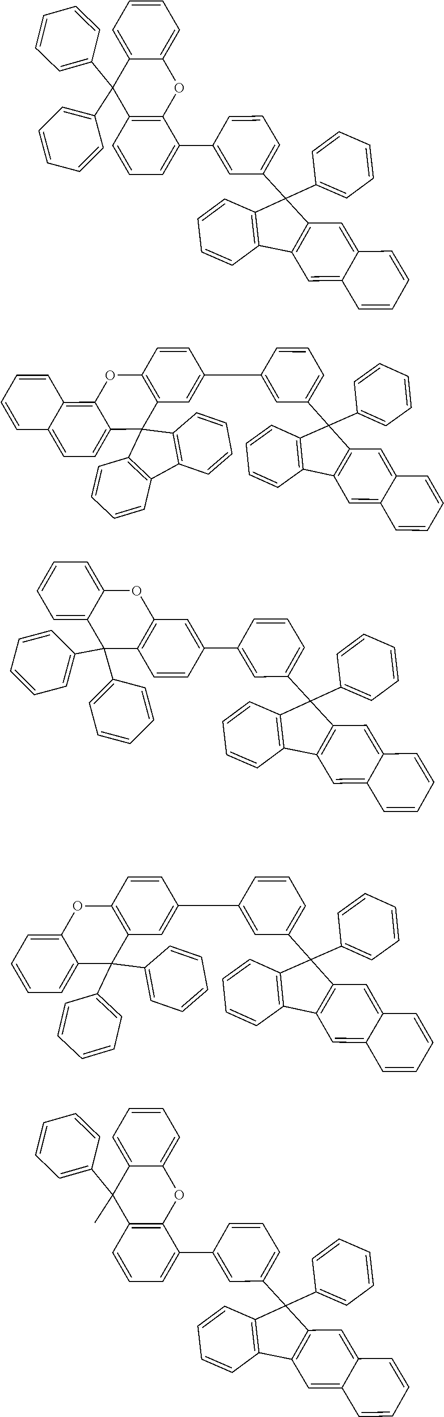 [SMILES: c1ccc(C2(c3cccc(-c4cccc5c4Oc4ccccc4C5(c4ccccc4)c4ccccc4)c3)c3ccccc3-c3cc4ccccc4cc32)cc1 ; c1ccc(C2(c3cccc(-c4ccc5c(c4)C4(c6ccccc6-c6ccccc64)c4ccc6ccccc6c4O5)c3)c3ccccc3-c3cc4ccccc4cc32)cc1 ; c1ccc(C2(c3ccccc3)c3ccccc3Oc3cc(-c4cccc(C5(c6ccccc6)c6ccccc6-c6cc7ccccc7cc65)c4)ccc32)cc1 ; c1ccc(C2(c3ccccc3)c3ccccc3Oc3ccc(-c4cccc(C5(c6ccccc6)c6ccccc6-c6cc7ccccc7cc65)c4)cc32)cc1 ; CC1(c2ccccc2)c2ccccc2Oc2c(-c3cccc(C4(c5ccccc5)c5ccccc5-c5cc6ccccc6cc54)c3)cccc21]

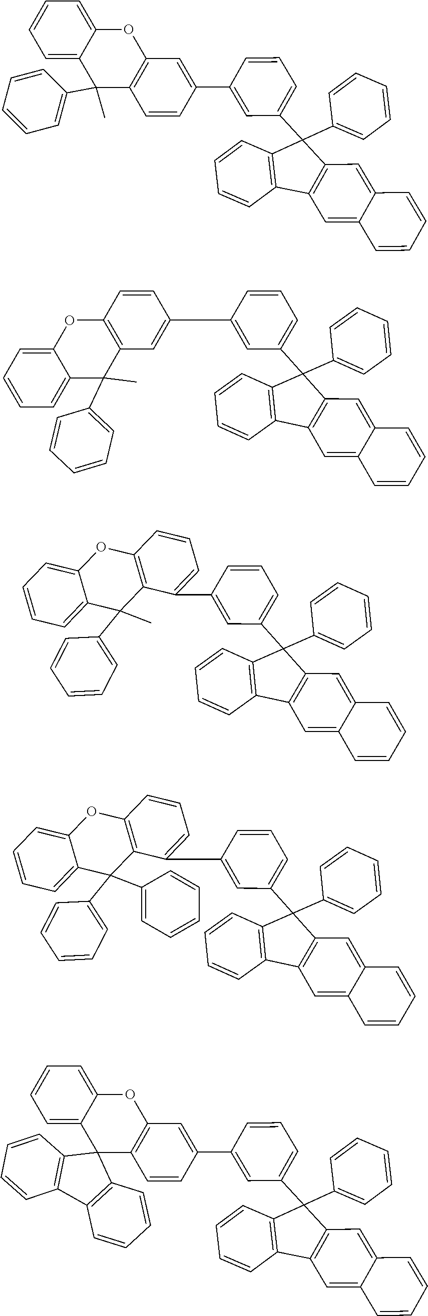 [SMILES: CC1(c2ccccc2)c2ccccc2Oc2cc(-c3cccc(C4(c5ccccc5)c5ccccc5-c5cc6ccccc6cc54)c3)ccc21 ; CC1(c2ccccc2)c2ccccc2Oc2ccc(-c3cccc(C4(c5ccccc5)c5ccccc5-c5cc6ccccc6cc54)c3)cc21 ; CC1(c2ccccc2)c2ccccc2Oc2cccc(-c3cccc(C4(c5ccccc5)c5ccccc5-c5cc6ccccc6cc54)c3)c21 ; c1ccc(C2(c3cccc(-c4cccc5c4C(c4ccccc4)(c4ccccc4)c4ccccc4O5)c3)c3ccccc3-c3cc4ccccc4cc32)cc1 ; c1ccc(C2(c3cccc(-c4ccc5c(c4)Oc4ccccc4C54c5ccccc5-c5ccccc54)c3)c3ccccc3-c3cc4ccccc4cc32)cc1]

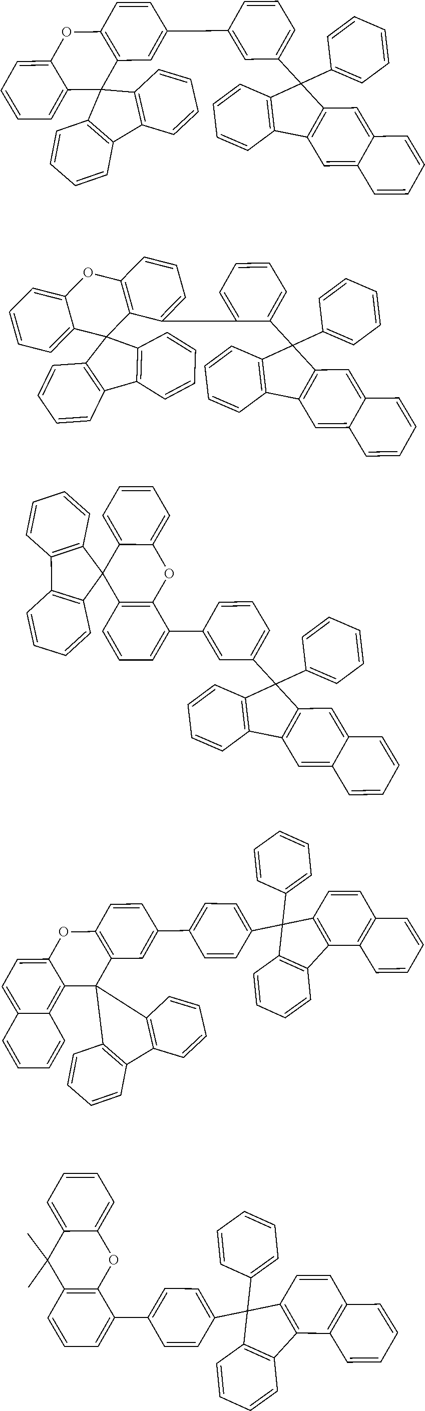 [SMILES: c1ccc(C2(c3cccc(-c4ccc5c(c4)C4(c6ccccc6O5)c5ccccc5-c5ccccc54)c3)c3ccccc3-c3cc4ccccc4cc32)cc1 ; c1ccc(C2(c3ccccc3-c3cccc4c3C3(c5ccccc5O4)c4ccccc4-c4ccccc43)c3ccccc3-c3cc4ccccc4cc32)cc1 ; c1ccc(C2(c3cccc(-c4cccc5c4Oc4ccccc4C54c5ccccc5-c5ccccc54)c3)c3ccccc3-c3cc4ccccc4cc32)cc1 ; c1ccc(C2(c3ccc(-c4ccc5c(c4)C4(c6ccccc6-c6ccccc64)c4c(ccc6ccccc46)O5)cc3)c3ccccc3-c3c2ccc2ccccc32)cc1 ; CC1(C)c2ccccc2Oc2c(-c3ccc(C4(c5ccccc5)c5ccccc5-c5c4ccc4ccccc54)cc3)cccc21]

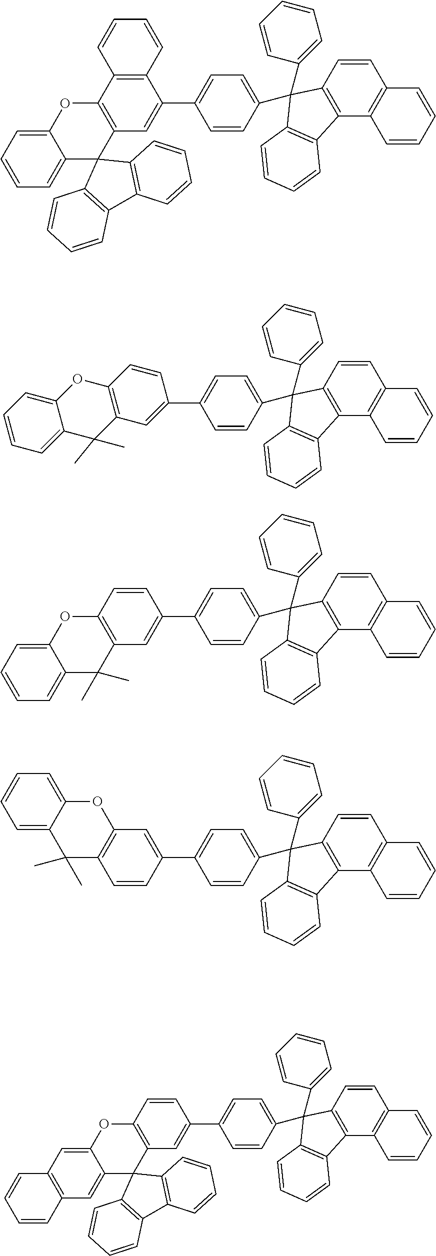

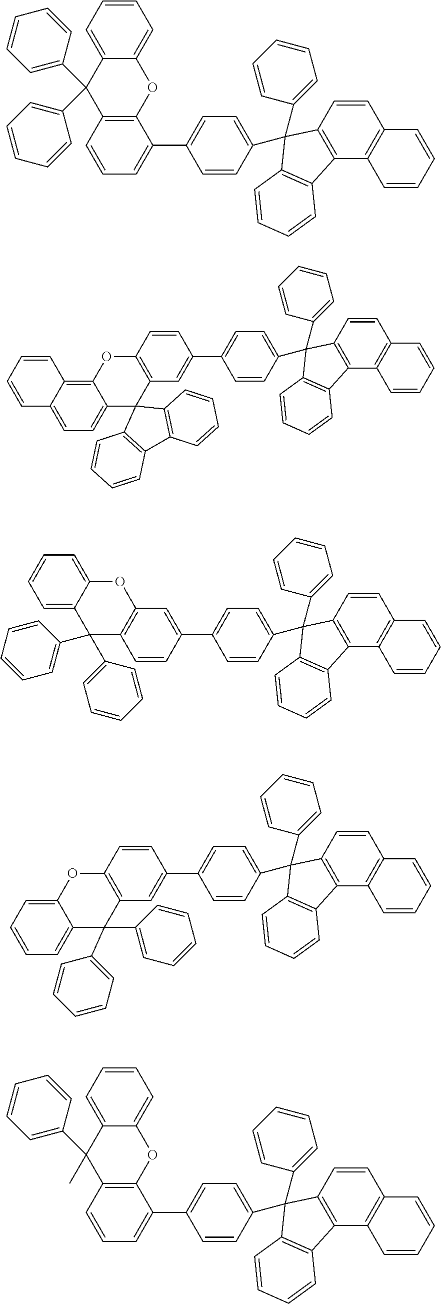

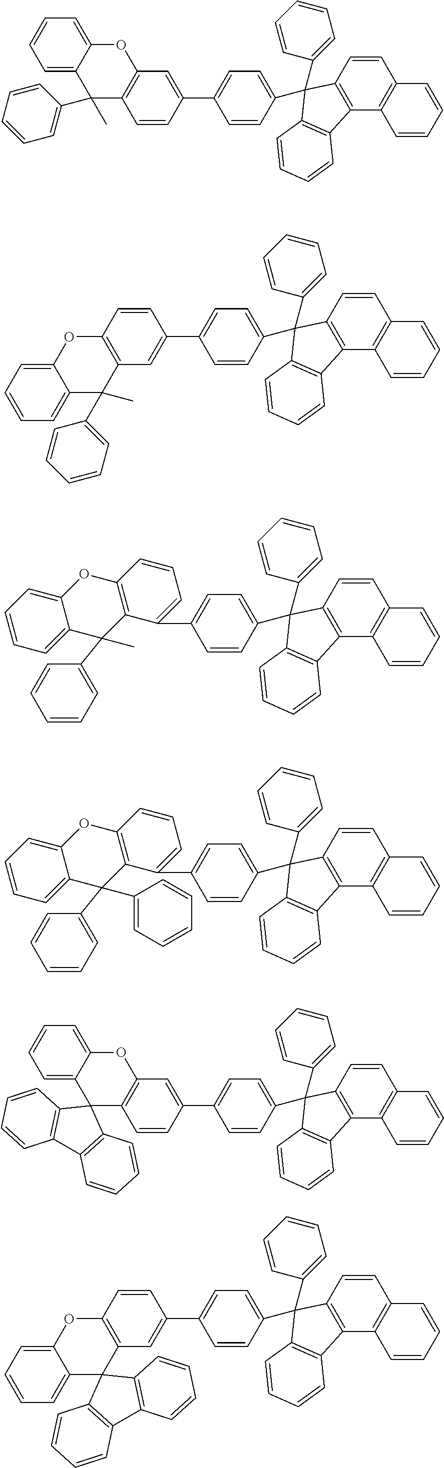

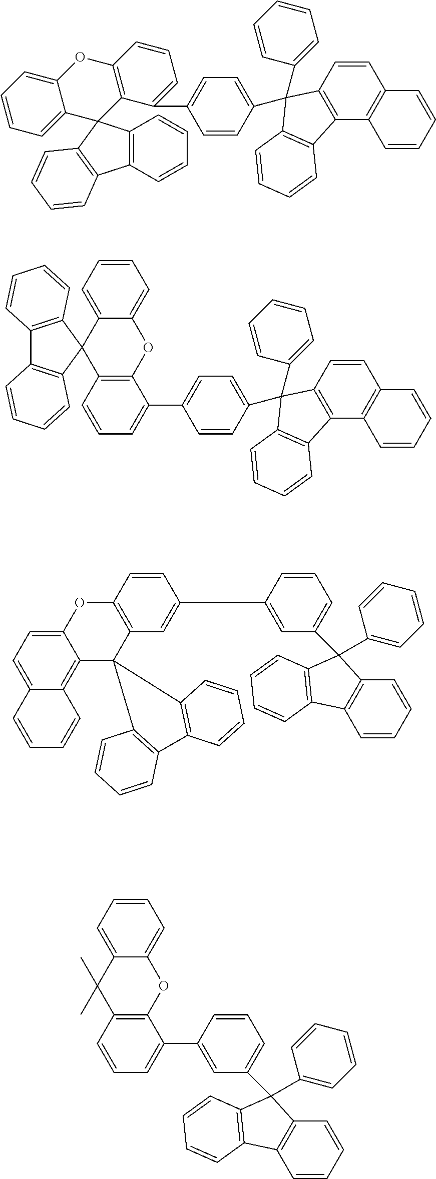 [SMILES: c1ccc(C2(c3ccc(-c4cccc5c4C4(c6ccccc6O5)c5ccccc5-c5ccccc54)cc3)c3ccccc3-c3c2ccc2ccccc32)cc1 ; c1ccc(C2(c3ccc(-c4cccc5c4Oc4ccccc4C54c5ccccc5-c5ccccc54)cc3)c3ccccc3-c3c2ccc2ccccc32)cc1 ; c1ccc(C2(c3cccc(-c4ccc5c(c4)C4(c6ccccc6-c6ccccc64)c4c(ccc6ccccc46)O5)c3)c3ccccc3-c3ccccc32)cc1 ; CC1(C)c2ccccc2Oc2c(-c3cccc(C4(c5ccccc5)c5ccccc5-c5ccccc54)c3)cccc21]

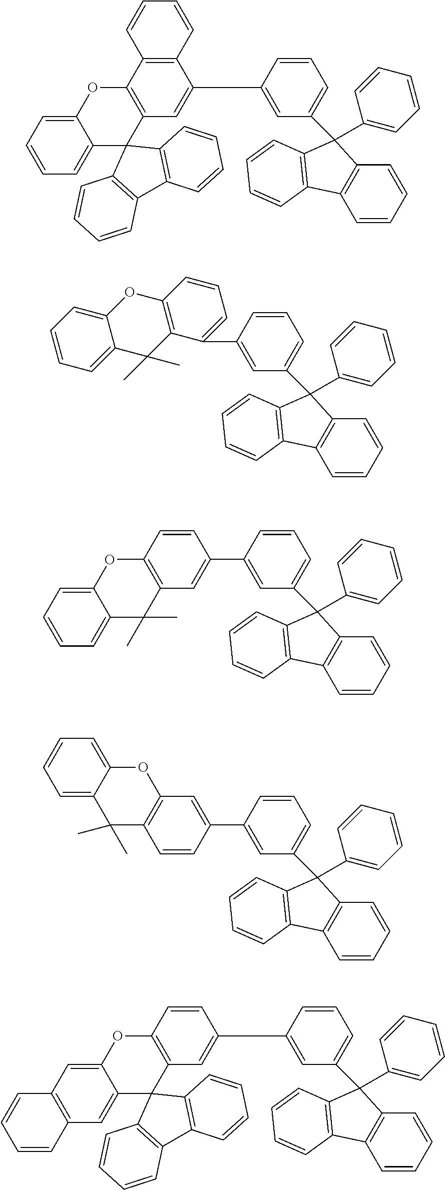 [SMILES: c1ccc(C2(c3cccc(-c4cc5c(c6ccccc46)Oc4ccccc4C54c5ccccc5-c5ccccc54)c3)c3ccccc3-c3ccccc32)cc1 ; CC1(C)c2ccccc2Oc2cccc(-c3cccc(C4(c5ccccc5)c5ccccc5-c5ccccc54)c3)c21 ; CC1(C)c2ccccc2Oc2ccc(-c3cccc(C4(c5ccccc5)c5ccccc5-c5ccccc54)c3)cc21 ; CC1(C)c2ccccc2Oc2cc(-c3cccc(C4(c5ccccc5)c5ccccc5-c5ccccc54)c3)ccc21 ; c1ccc(C2(c3cccc(-c4ccc5c(c4)C4(c6cc7ccccc7cc6O5)c5ccccc5-c5ccccc54)c3)c3ccccc3-c3ccccc32)cc1]

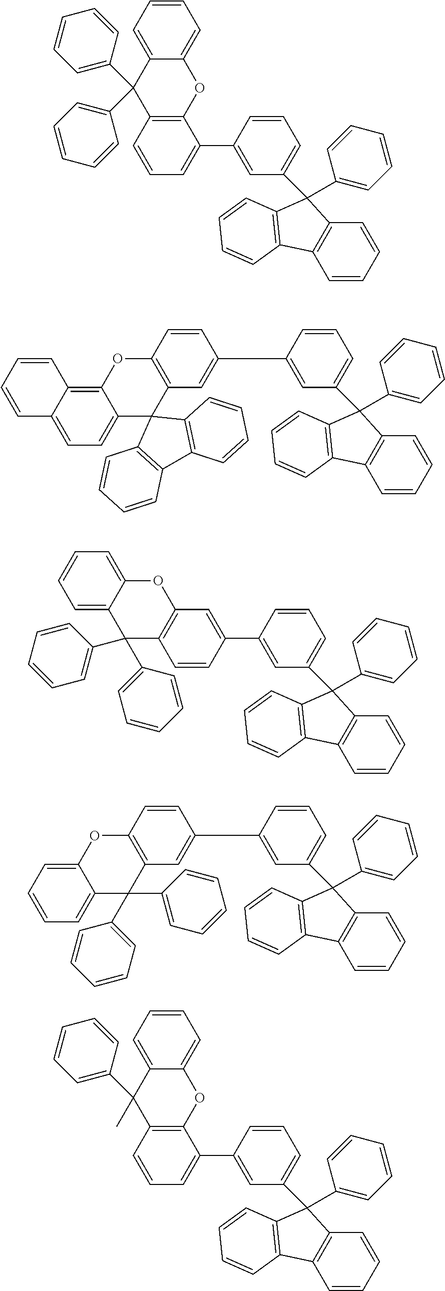

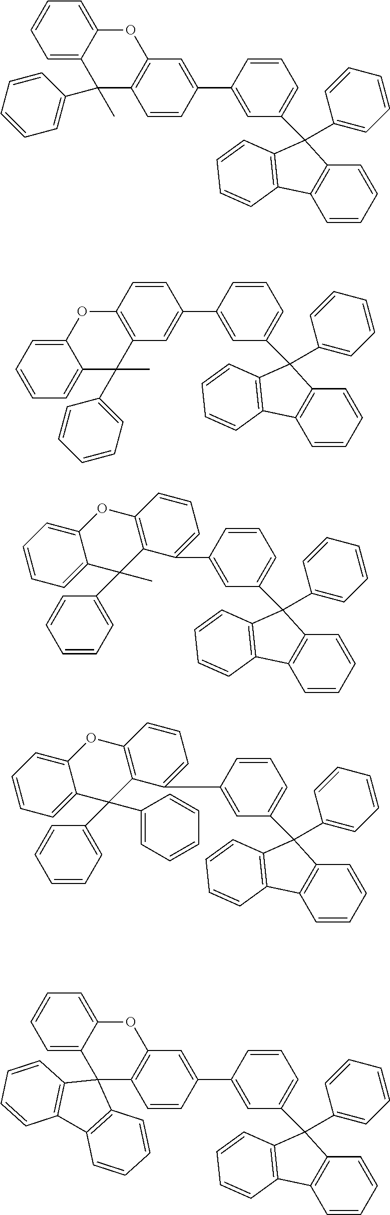 [SMILES: CC1(c2ccccc2)c2ccccc2Oc2cc(-c3cccc(C4(c5ccccc5)c5ccccc5-c5ccccc54)c3)ccc21 ; CC1(c2ccccc2)c2ccccc2Oc2ccc(-c3cccc(C4(c5ccccc5)c5ccccc5-c5ccccc54)c3)cc21 ; CC1(c2ccccc2)c2ccccc2Oc2cccc(-c3cccc(C4(c5ccccc5)c5ccccc5-c5ccccc54)c3)c21 ; c1ccc(C2(c3cccc(-c4cccc5c4C(c4ccccc4)(c4ccccc4)c4ccccc4O5)c3)c3ccccc3-c3ccccc32)cc1 ; c1ccc(C2(c3cccc(-c4ccc5c(c4)Oc4ccccc4C54c5ccccc5-c5ccccc54)c3)c3ccccc3-c3ccccc32)cc1]

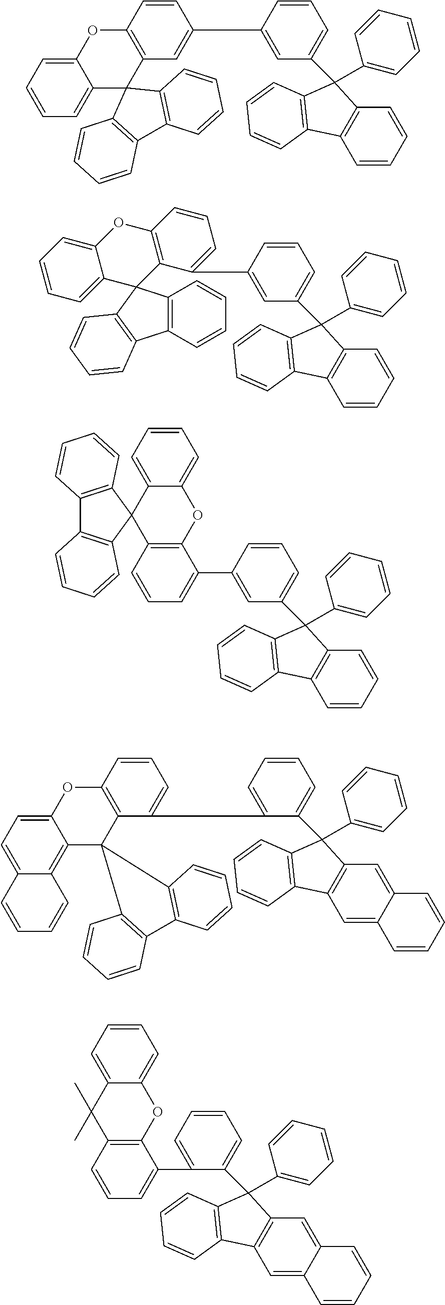

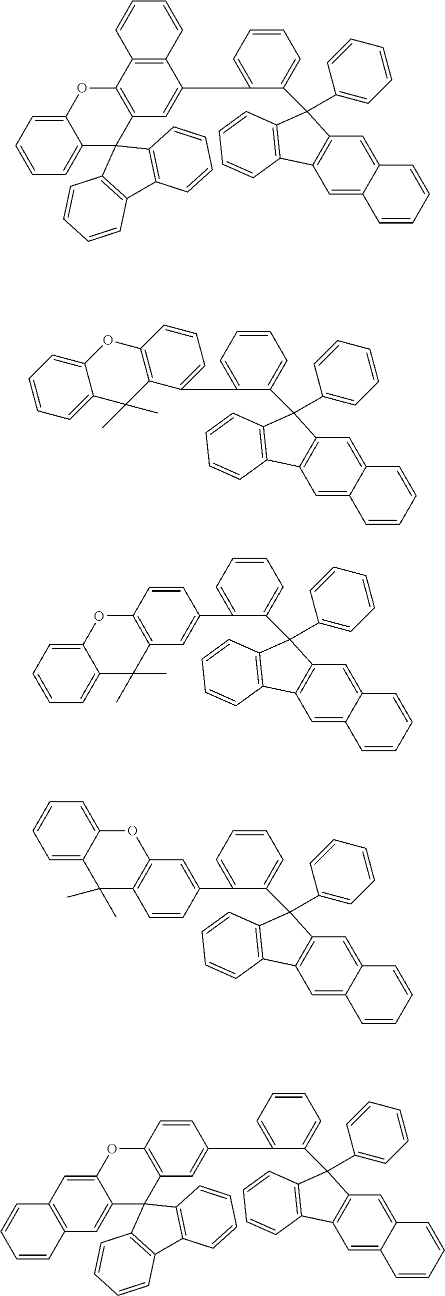 [SMILES: c1ccc(C2(c3ccccc3-c3cc4c(c5ccccc35)Oc3ccccc3C43c4ccccc4-c4ccccc43)c3ccccc3-c3cc4ccccc4cc32)cc1 ; CC1(C)c2ccccc2Oc2cccc(-c3ccccc3C3(c4ccccc4)c4ccccc4-c4cc5ccccc5cc43)c21 ; CC1(C)c2ccccc2Oc2ccc(-c3ccccc3C3(c4ccccc4)c4ccccc4-c4cc5ccccc5cc43)cc21 ; CC1(C)c2ccccc2Oc2cc(-c3ccccc3C3(c4ccccc4)c4ccccc4-c4cc5ccccc5cc43)ccc21 ; c1ccc(C2(c3ccccc3-c3ccc4c(c3)C3(c5cc6ccccc6cc5O4)c4ccccc4-c4ccccc43)c3ccccc3-c3cc4ccccc4cc32)cc1]

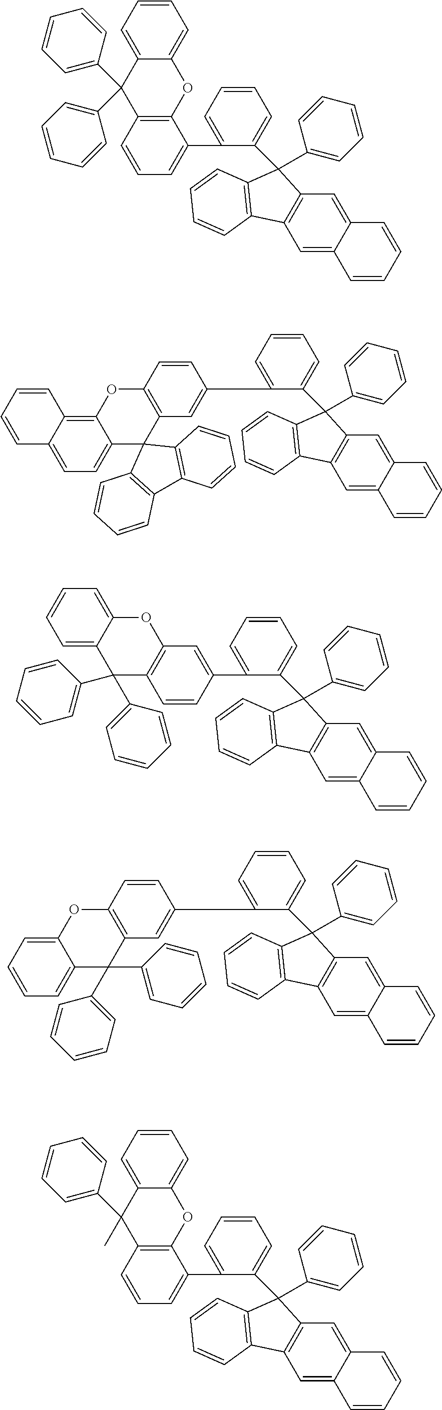 [SMILES: c1ccc(C2(c3ccccc3)c3ccccc3Oc3c(-c4ccccc4C4(c5ccccc5)c5ccccc5-c5cc6ccccc6cc54)cccc32)cc1 ; c1ccc(C2(c3ccccc3-c3ccc4c(c3)C3(c5ccccc5-c5ccccc53)c3ccc5ccccc5c3O4)c3ccccc3-c3cc4ccccc4cc32)cc1 ; c1ccc(C2(c3ccccc3)c3ccccc3Oc3cc(-c4ccccc4C4(c5ccccc5)c5ccccc5-c5cc6ccccc6cc54)ccc32)cc1 ; c1ccc(C2(c3ccccc3)c3ccccc3Oc3ccc(-c4ccccc4C4(c5ccccc5)c5ccccc5-c5cc6ccccc6cc54)cc32)cc1 ; CC1(c2ccccc2)c2ccccc2Oc2c(-c3ccccc3C3(c4ccccc4)c4ccccc4-c4cc5ccccc5cc43)cccc21]

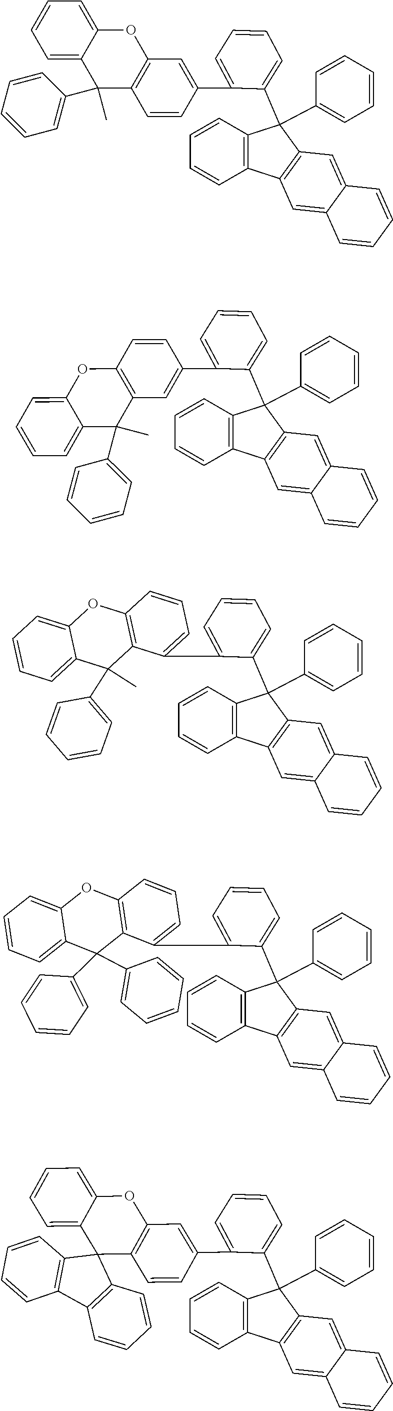 [SMILES: CC1(c2ccccc2)c2ccccc2Oc2cc(-c3ccccc3C3(c4ccccc4)c4ccccc4-c4cc5ccccc5cc43)ccc21 ; CC1(c2ccccc2)c2ccccc2Oc2ccc(-c3ccccc3C3(c4ccccc4)c4ccccc4-c4cc5ccccc5cc43)cc21 ; CC1(c2ccccc2)c2ccccc2Oc2cccc(-c3ccccc3C3(c4ccccc4)c4ccccc4-c4cc5ccccc5cc43)c21 ; c1ccc(C2(c3ccccc3-c3cccc4c3C(c3ccccc3)(c3ccccc3)c3ccccc3O4)c3ccccc3-c3cc4ccccc4cc32)cc1 ; c1ccc(C2(c3ccccc3-c3ccc4c(c3)Oc3ccccc3C43c4ccccc4-c4ccccc43)c3ccccc3-c3cc4ccccc4cc32)cc1]

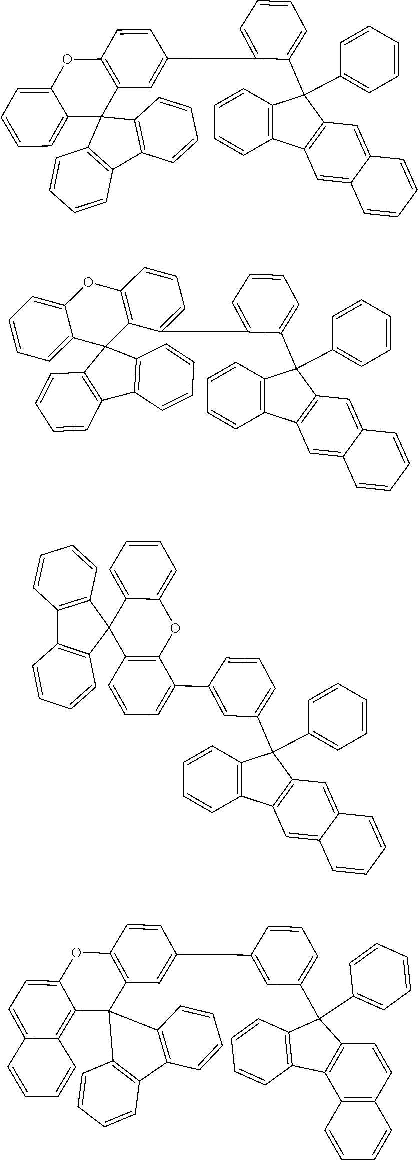 [SMILES: c1ccc(C2(c3ccccc3-c3ccc4c(c3)C3(c5ccccc5O4)c4ccccc4-c4ccccc43)c3ccccc3-c3cc4ccccc4cc32)cc1 ; c1ccc(C2(c3ccccc3-c3cccc4c3C3(c5ccccc5O4)c4ccccc4-c4ccccc43)c3ccccc3-c3cc4ccccc4cc32)cc1 ; c1ccc(C2(c3cccc(-c4cccc5c4Oc4ccccc4C54c5ccccc5-c5ccccc54)c3)c3ccccc3-c3cc4ccccc4cc32)cc1 ; c1ccc(C2(c3cccc(-c4ccc5c(c4)C4(c6ccccc6-c6ccccc64)c4c(ccc6ccccc46)O5)c3)c3ccccc3-c3c2ccc2ccccc32)cc1]

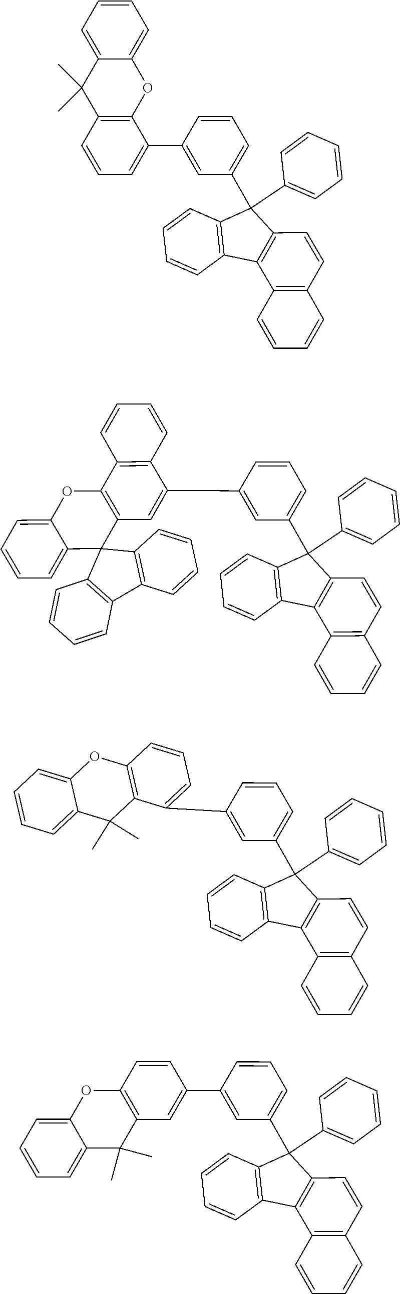 [SMILES: CC1(C)c2ccccc2Oc2c(-c3cccc(C4(c5ccccc5)c5ccccc5-c5c4ccc4ccccc54)c3)cccc21 ; c1ccc(C2(c3cccc(-c4cc5c(c6ccccc46)Oc4ccccc4C54c5ccccc5-c5ccccc54)c3)c3ccccc3-c3c2ccc2ccccc32)cc1 ; CC1(C)c2ccccc2Oc2cccc(-c3cccc(C4(c5ccccc5)c5ccccc5-c5c4ccc4ccccc54)c3)c21 ; CC1(C)c2ccccc2Oc2ccc(-c3cccc(C4(c5ccccc5)c5ccccc5-c5c4ccc4ccccc54)c3)cc21]

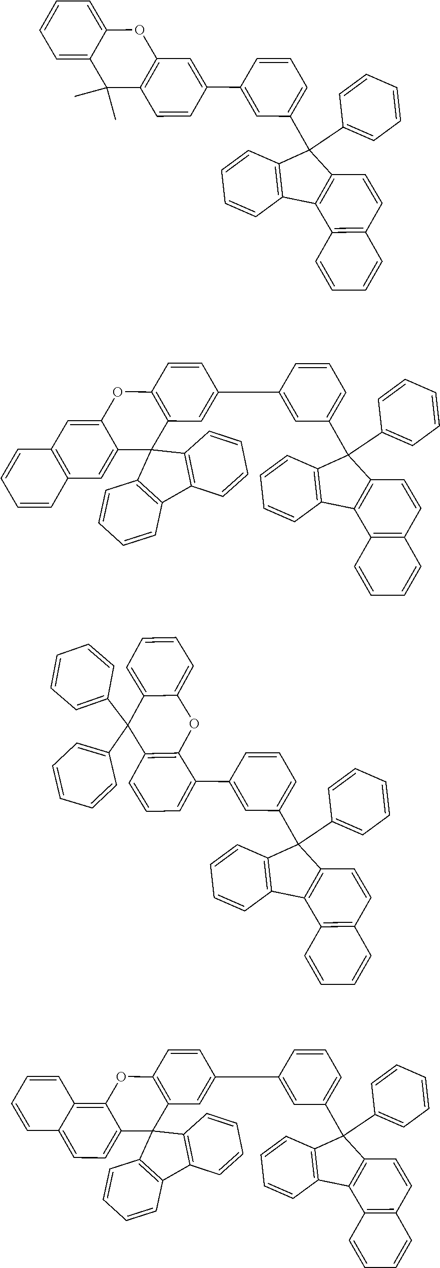 [SMILES: CC1(C)c2ccccc2Oc2cc(-c3cccc(C4(c5ccccc5)c5ccccc5-c5c4ccc4ccccc54)c3)ccc21 ; c1ccc(C2(c3cccc(-c4ccc5c(c4)C4(c6cc7ccccc7cc6O5)c5ccccc5-c5ccccc54)c3)c3ccccc3-c3c2ccc2ccccc32)cc1 ; c1ccc(C2(c3ccccc3)c3ccccc3Oc3c(-c4cccc(C5(c6ccccc6)c6ccccc6-c6c5ccc5ccccc65)c4)cccc32)cc1 ; c1ccc(C2(c3cccc(-c4ccc5c(c4)C4(c6ccccc6-c6ccccc64)c4ccc6ccccc6c4O5)c3)c3ccccc3-c3c2ccc2ccccc32)cc1]

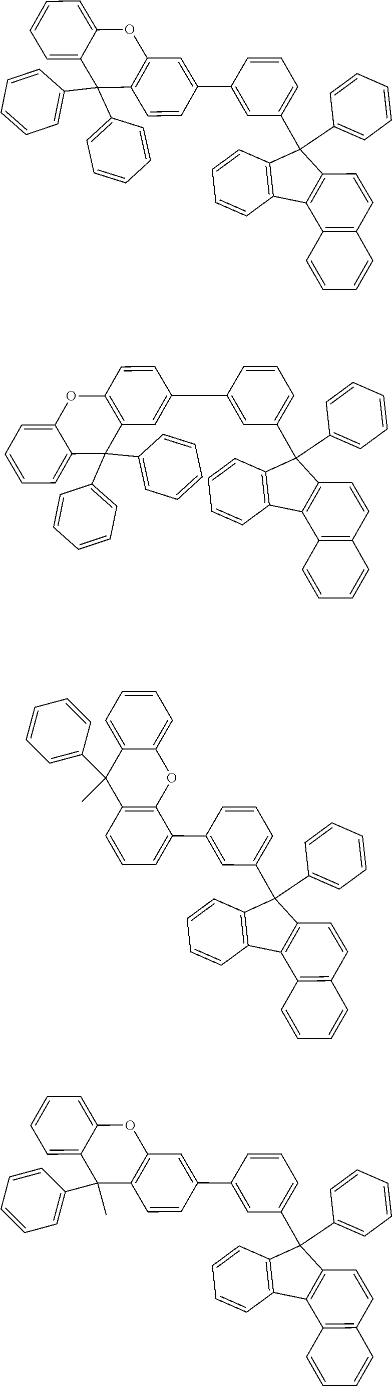 [SMILES: c1ccc(C2(c3ccccc3)c3ccccc3Oc3cc(-c4cccc(C5(c6ccccc6)c6ccccc6-c6c5ccc5ccccc65)c4)ccc32)cc1 ; c1ccc(C2(c3ccccc3)c3ccccc3Oc3ccc(-c4cccc(C5(c6ccccc6)c6ccccc6-c6c5ccc5ccccc65)c4)cc32)cc1 ; CC1(c2ccccc2)c2ccccc2Oc2c(-c3cccc(C4(c5ccccc5)c5ccccc5-c5c4ccc4ccccc54)c3)cccc21 ; CC1(c2ccccc2)c2ccccc2Oc2cc(-c3cccc(C4(c5ccccc5)c5ccccc5-c5c4ccc4ccccc54)c3)ccc21]

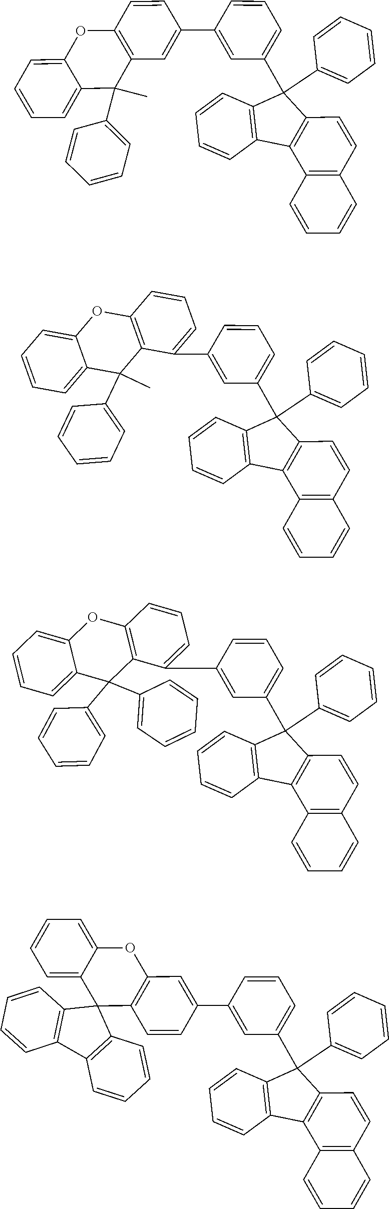 [SMILES: CC1(c2ccccc2)c2ccccc2Oc2ccc(-c3cccc(C4(c5ccccc5)c5ccccc5-c5c4ccc4ccccc54)c3)cc21 ; CC1(c2ccccc2)c2ccccc2Oc2cccc(-c3cccc(C4(c5ccccc5)c5ccccc5-c5c4ccc4ccccc54)c3)c21 ; c1ccc(C2(c3cccc(-c4cccc5c4C(c4ccccc4)(c4ccccc4)c4ccccc4O5)c3)c3ccccc3-c3c2ccc2ccccc32)cc1 ; c1ccc(C2(c3cccc(-c4ccc5c(c4)Oc4ccccc4C54c5ccccc5-c5ccccc54)c3)c3ccccc3-c3c2ccc2ccccc32)cc1]

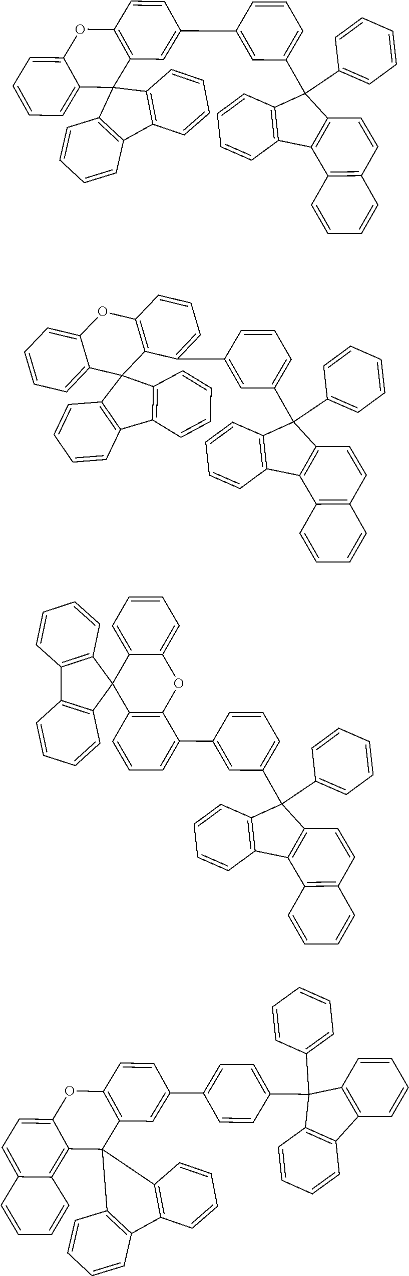

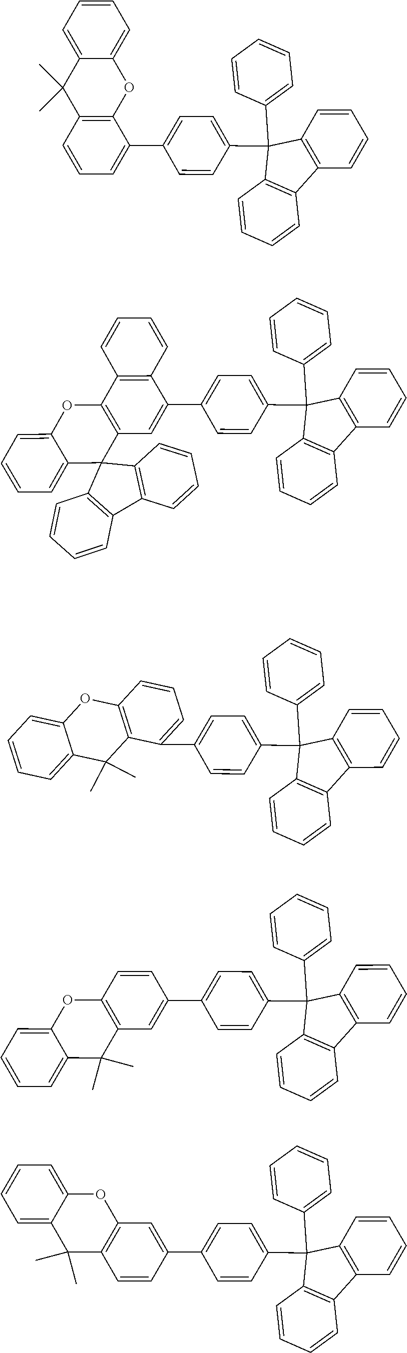 [SMILES: CC1(C)c2ccccc2Oc2c(-c3ccc(C4(c5ccccc5)c5ccccc5-c5ccccc54)cc3)cccc21 ; c1ccc(C2(c3ccc(-c4cc5c(c6ccccc46)Oc4ccccc4C54c5ccccc5-c5ccccc54)cc3)c3ccccc3-c3ccccc32)cc1 ; CC1(C)c2ccccc2Oc2cccc(-c3ccc(C4(c5ccccc5)c5ccccc5-c5ccccc54)cc3)c21 ; CC1(C)c2ccccc2Oc2ccc(-c3ccc(C4(c5ccccc5)c5ccccc5-c5ccccc54)cc3)cc21 ; CC1(C)c2ccccc2Oc2cc(-c3ccc(C4(c5ccccc5)c5ccccc5-c5ccccc54)cc3)ccc21]

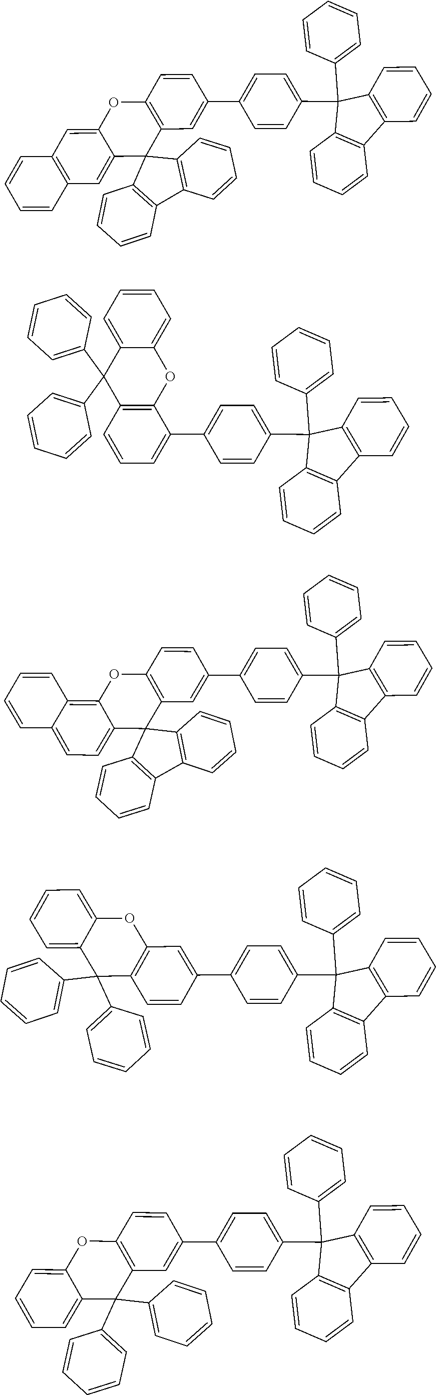

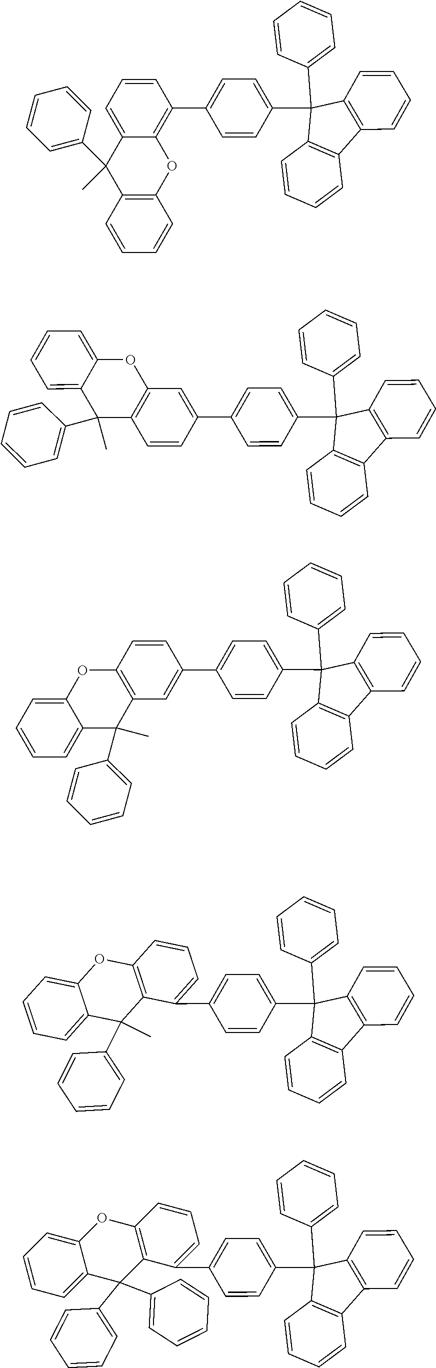 [SMILES: CC1(c2ccccc2)c2ccccc2Oc2c(-c3ccc(C4(c5ccccc5)c5ccccc5-c5ccccc54)cc3)cccc21 ; CC1(c2ccccc2)c2ccccc2Oc2cc(-c3ccc(C4(c5ccccc5)c5ccccc5-c5ccccc54)cc3)ccc21 ; CC1(c2ccccc2)c2ccccc2Oc2ccc(-c3ccc(C4(c5ccccc5)c5ccccc5-c5ccccc54)cc3)cc21 ; CC1(c2ccccc2)c2ccccc2Oc2cccc(-c3ccc(C4(c5ccccc5)c5ccccc5-c5ccccc54)cc3)c21 ; c1ccc(C2(c3ccc(-c4cccc5c4C(c4ccccc4)(c4ccccc4)c4ccccc4O5)cc3)c3ccccc3-c3ccccc32)cc1]

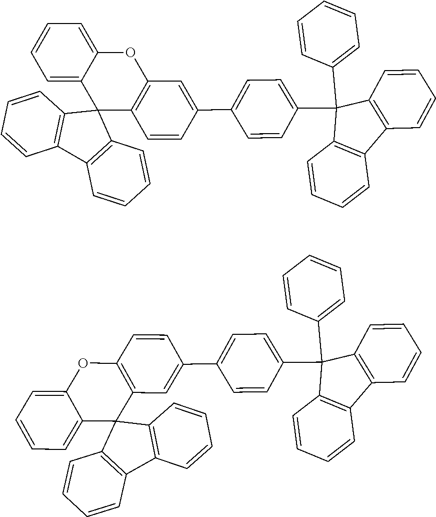 [SMILES: c1ccc(C2(c3ccc(-c4ccc5c(c4)Oc4ccccc4C54c5ccccc5-c5ccccc54)cc3)c3ccccc3-c3ccccc32)cc1 ; c1ccc(C2(c3ccc(-c4ccc5c(c4)C4(c6ccccc6O5)c5ccccc5-c5ccccc54)cc3)c3ccccc3-c3ccccc32)cc1]

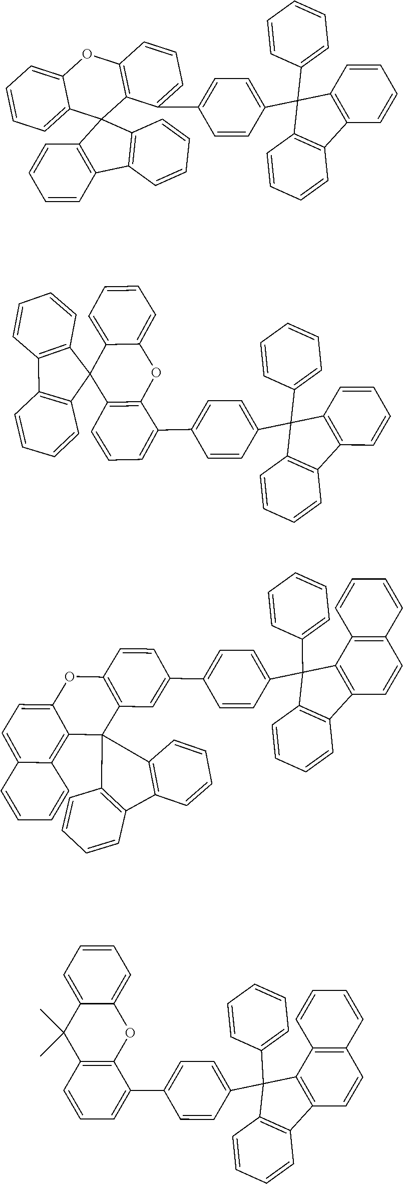 [SMILES: c1ccc(C2(c3ccc(-c4cccc5c4C4(c6ccccc6O5)c5ccccc5-c5ccccc54)cc3)c3ccccc3-c3ccccc32)cc1 ; c1ccc(C2(c3ccc(-c4cccc5c4Oc4ccccc4C54c5ccccc5-c5ccccc54)cc3)c3ccccc3-c3ccccc32)cc1 ; c1ccc(C2(c3ccc(-c4ccc5c(c4)C4(c6ccccc6-c6ccccc64)c4c(ccc6ccccc46)O5)cc3)c3ccccc3-c3ccc4ccccc4c32)cc1 ; CC1(C)c2ccccc2Oc2c(-c3ccc(C4(c5ccccc5)c5ccccc5-c5ccc6ccccc6c54)cc3)cccc21]

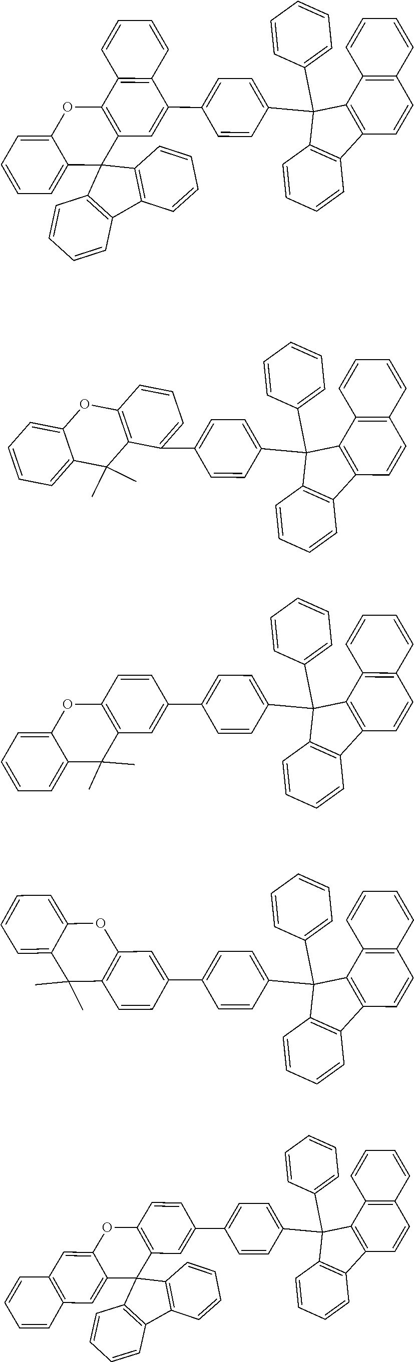 [SMILES: c1ccc(C2(c3ccc(-c4cc5c(c6ccccc46)Oc4ccccc4C54c5ccccc5-c5ccccc54)cc3)c3ccccc3-c3ccc4ccccc4c32)cc1 ; CC1(C)c2ccccc2Oc2cccc(-c3ccc(C4(c5ccccc5)c5ccccc5-c5ccc6ccccc6c54)cc3)c21 ; CC1(C)c2ccccc2Oc2ccc(-c3ccc(C4(c5ccccc5)c5ccccc5-c5ccc6ccccc6c54)cc3)cc21 ; CC1(C)c2ccccc2Oc2cc(-c3ccc(C4(c5ccccc5)c5ccccc5-c5ccc6ccccc6c54)cc3)ccc21 ; c1ccc(C2(c3ccc(-c4ccc5c(c4)C4(c6cc7ccccc7cc6O5)c5ccccc5-c5ccccc54)cc3)c3ccccc3-c3ccc4ccccc4c32)cc1]

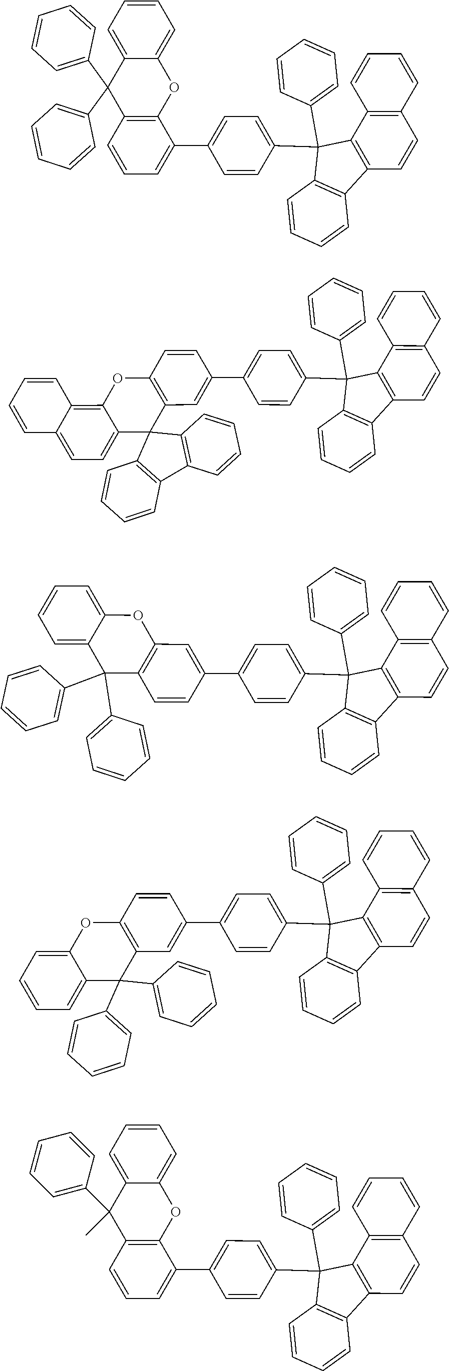

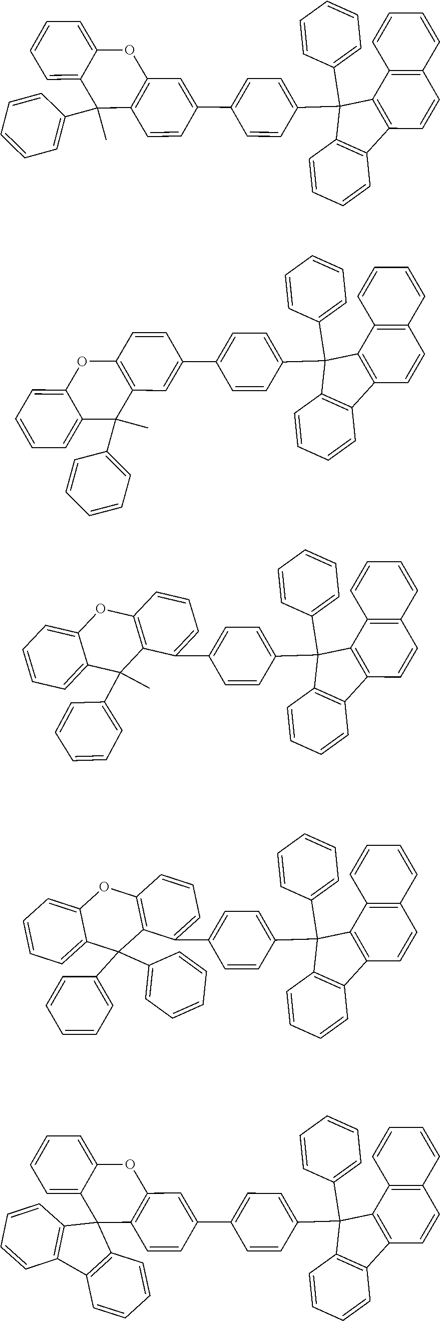 [SMILES: CC1(c2ccccc2)c2ccccc2Oc2cc(-c3ccc(C4(c5ccccc5)c5ccccc5-c5ccc6ccccc6c54)cc3)ccc21 ; CC1(c2ccccc2)c2ccccc2Oc2ccc(-c3ccc(C4(c5ccccc5)c5ccccc5-c5ccc6ccccc6c54)cc3)cc21 ; CC1(c2ccccc2)c2ccccc2Oc2cccc(-c3ccc(C4(c5ccccc5)c5ccccc5-c5ccc6ccccc6c54)cc3)c21 ; c1ccc(C2(c3ccccc3)c3ccccc3Oc3cccc(-c4ccc(C5(c6ccccc6)c6ccccc6-c6ccc7ccccc7c65)cc4)c32)cc1 ; c1ccc(C2(c3ccc(-c4ccc5c(c4)Oc4ccccc4C54c5ccccc5-c5ccccc54)cc3)c3ccccc3-c3ccc4ccccc4c32)cc1]

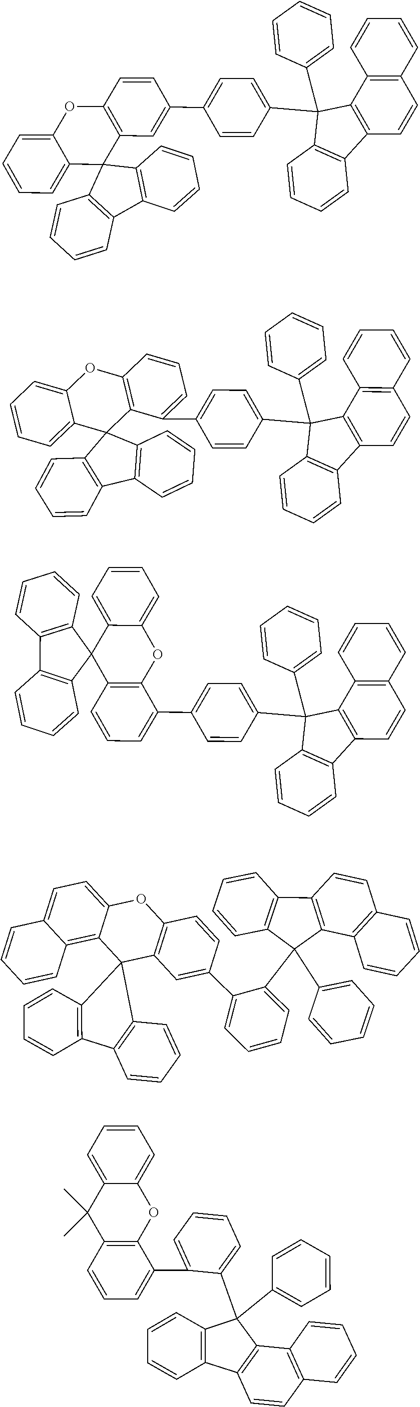 [SMILES: c1ccc(C2(c3ccc(-c4ccc5c(c4)C4(c6ccccc6O5)c5ccccc5-c5ccccc54)cc3)c3ccccc3-c3ccc4ccccc4c32)cc1 ; c1ccc(C2(c3ccc(-c4cccc5c4C4(c6ccccc6O5)c5ccccc5-c5ccccc54)cc3)c3ccccc3-c3ccc4ccccc4c32)cc1 ; c1ccc(C2(c3ccc(-c4cccc5c4Oc4ccccc4C54c5ccccc5-c5ccccc54)cc3)c3ccccc3-c3ccc4ccccc4c32)cc1 ; c1ccc(C2(c3ccccc3-c3ccc4c(c3)C3(c5ccccc5-c5ccccc53)c3c(ccc5ccccc35)O4)c3ccccc3-c3ccc4ccccc4c32)cc1 ; CC1(C)c2ccccc2Oc2c(-c3ccccc3C3(c4ccccc4)c4ccccc4-c4ccc5ccccc5c43)cccc21]

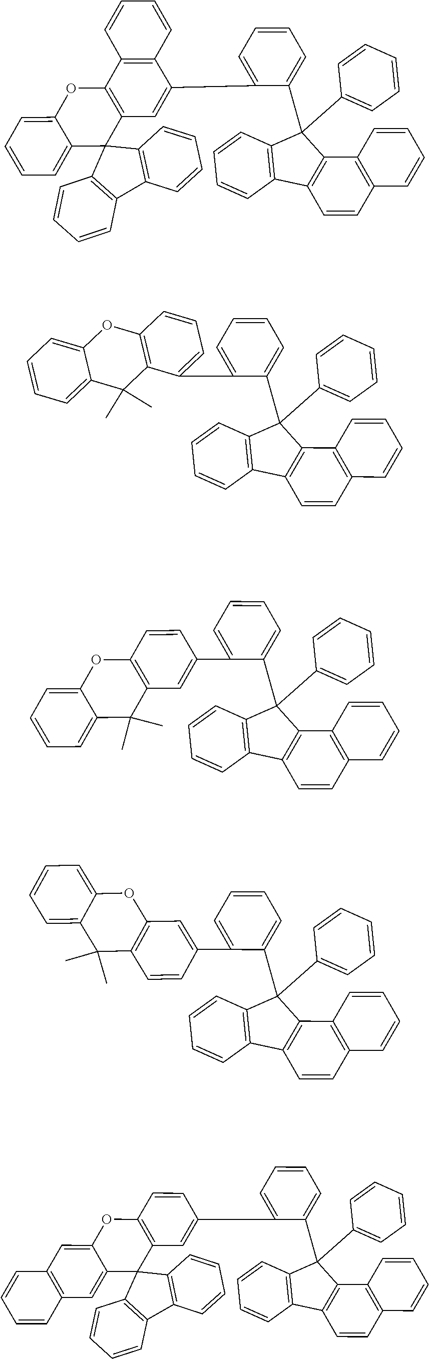 [SMILES: c1ccc(C2(c3ccccc3-c3cc4c(c5ccccc35)Oc3ccccc3C43c4ccccc4-c4ccccc43)c3ccccc3-c3ccc4ccccc4c32)cc1 ; CC1(C)c2ccccc2Oc2cccc(-c3ccccc3C3(c4ccccc4)c4ccccc4-c4ccc5ccccc5c43)c21 ; CC1(C)c2ccccc2Oc2ccc(-c3ccccc3C3(c4ccccc4)c4ccccc4-c4ccc5ccccc5c43)cc21 ; CC1(C)c2ccccc2Oc2cc(-c3ccccc3C3(c4ccccc4)c4ccccc4-c4ccc5ccccc5c43)ccc21 ; c1ccc(C2(c3ccccc3-c3ccc4c(c3)C3(c5cc6ccccc6cc5O4)c4ccccc4-c4ccccc43)c3ccccc3-c3ccc4ccccc4c32)cc1]

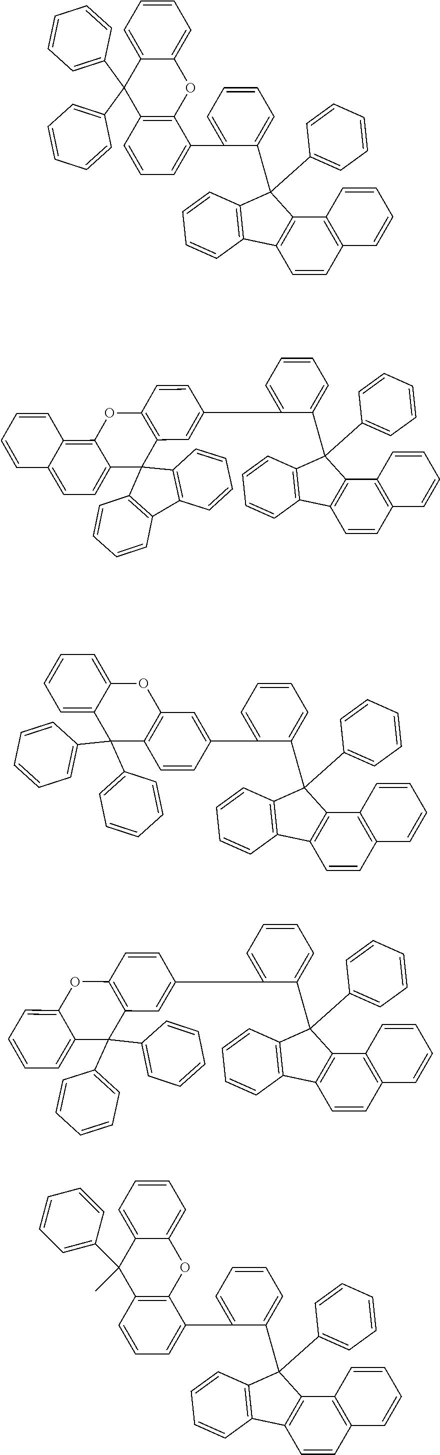 [SMILES: c1ccc(C2(c3ccccc3)c3ccccc3Oc3c(-c4ccccc4C4(c5ccccc5)c5ccccc5-c5ccc6ccccc6c54)cccc32)cc1 ; c1ccc(C2(c3ccccc3-c3ccc4c(c3)C3(c5ccccc5-c5ccccc53)c3ccc5ccccc5c3O4)c3ccccc3-c3ccc4ccccc4c32)cc1 ; c1ccc(C2(c3ccccc3)c3ccccc3Oc3cc(-c4ccccc4C4(c5ccccc5)c5ccccc5-c5ccc6ccccc6c54)ccc32)cc1 ; c1ccc(C2(c3ccccc3)c3ccccc3Oc3ccc(-c4ccccc4C4(c5ccccc5)c5ccccc5-c5ccc6ccccc6c54)cc32)cc1 ; CC1(c2ccccc2)c2ccccc2Oc2c(-c3ccccc3C3(c4ccccc4)c4ccccc4-c4ccc5ccccc5c43)cccc21]

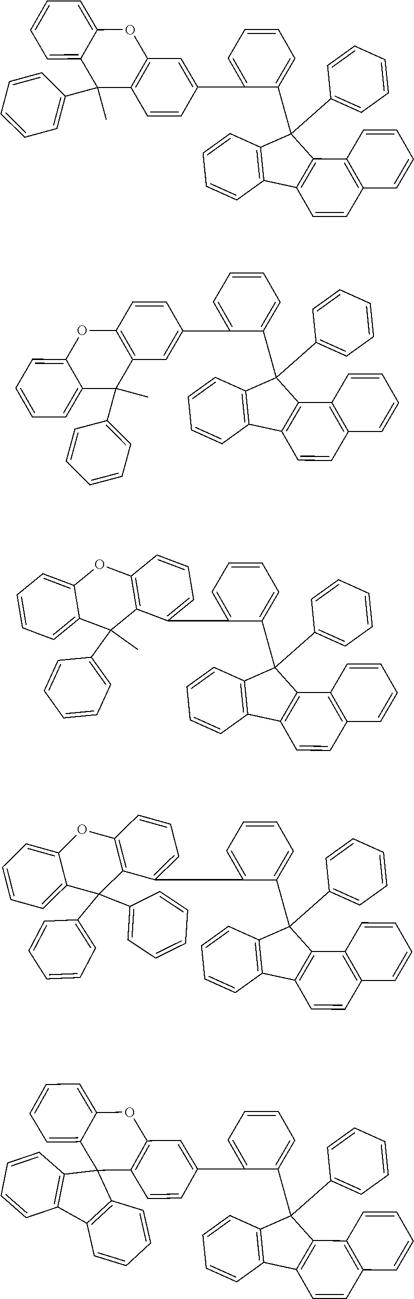 [SMILES: CC1(c2ccccc2)c2ccccc2Oc2cc(-c3ccccc3C3(c4ccccc4)c4ccccc4-c4ccc5ccccc5c43)ccc21 ; CC1(c2ccccc2)c2ccccc2Oc2ccc(-c3ccccc3C3(c4ccccc4)c4ccccc4-c4ccc5ccccc5c43)cc21 ; CC1(c2ccccc2)c2ccccc2Oc2cccc(-c3ccccc3C3(c4ccccc4)c4ccccc4-c4ccc5ccccc5c43)c21 ; c1ccc(C2(c3ccccc3)c3ccccc3Oc3cccc(-c4ccccc4C4(c5ccccc5)c5ccccc5-c5ccc6ccccc6c54)c32)cc1 ; c1ccc(C2(c3ccccc3-c3ccc4c(c3)Oc3ccccc3C43c4ccccc4-c4ccccc43)c3ccccc3-c3ccc4ccccc4c32)cc1]

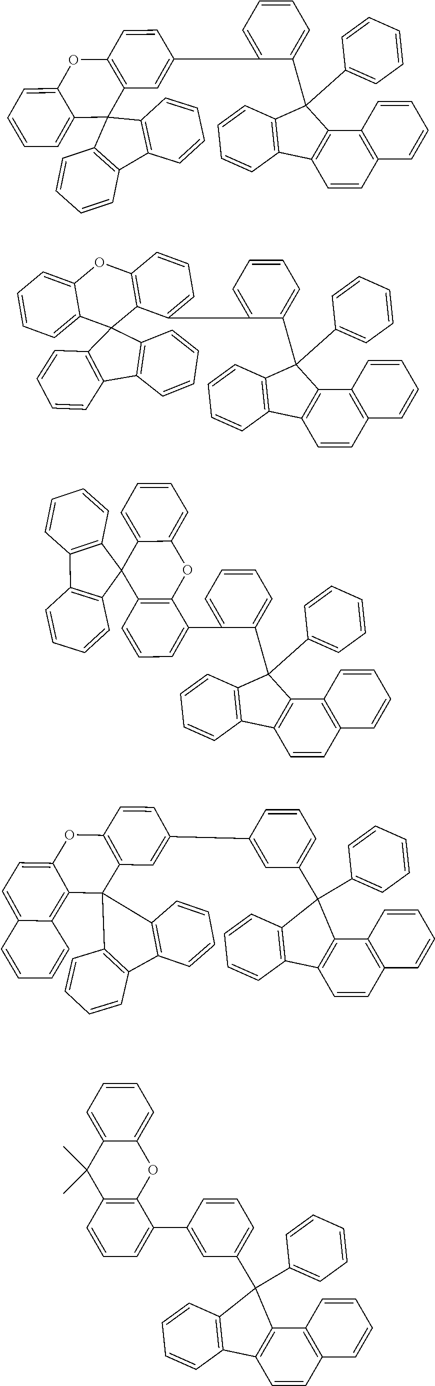 [SMILES: c1ccc(C2(c3ccccc3-c3ccc4c(c3)C3(c5ccccc5O4)c4ccccc4-c4ccccc43)c3ccccc3-c3ccc4ccccc4c32)cc1 ; c1ccc(C2(c3ccccc3-c3cccc4c3C3(c5ccccc5O4)c4ccccc4-c4ccccc43)c3ccccc3-c3ccc4ccccc4c32)cc1 ; c1ccc(C2(c3ccccc3-c3cccc4c3Oc3ccccc3C43c4ccccc4-c4ccccc43)c3ccccc3-c3ccc4ccccc4c32)cc1 ; c1ccc(C2(c3cccc(-c4ccc5c(c4)C4(c6ccccc6-c6ccccc64)c4c(ccc6ccccc46)O5)c3)c3ccccc3-c3ccc4ccccc4c32)cc1 ; CC1(C)c2ccccc2Oc2c(-c3cccc(C4(c5ccccc5)c5ccccc5-c5ccc6ccccc6c54)c3)cccc21]

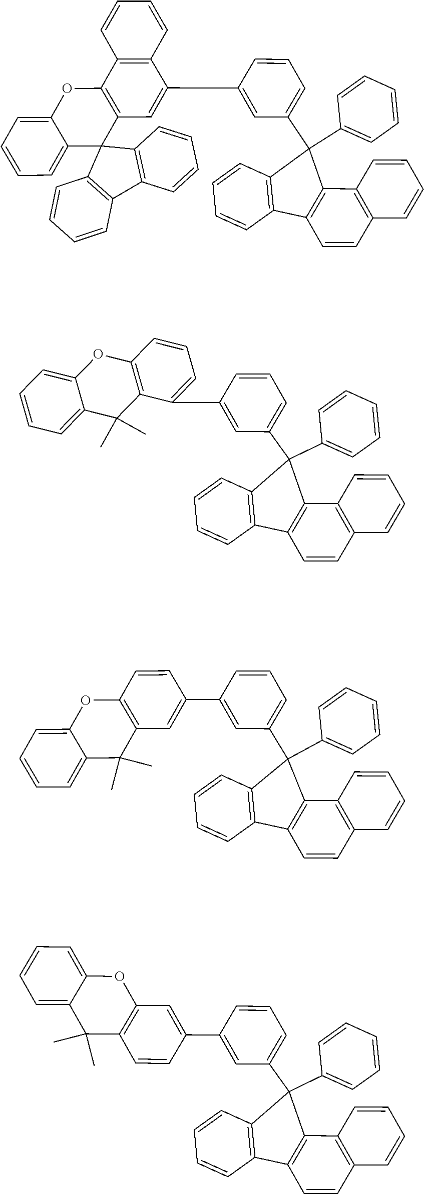 [SMILES: c1ccc(C2(c3cccc(-c4cc5c(c6ccccc46)Oc4ccccc4C54c5ccccc5-c5ccccc54)c3)c3ccccc3-c3ccc4ccccc4c32)cc1 ; CC1(C)c2ccccc2Oc2cccc(-c3cccc(C4(c5ccccc5)c5ccccc5-c5ccc6ccccc6c54)c3)c21 ; CC1(C)c2ccccc2Oc2ccc(-c3cccc(C4(c5ccccc5)c5ccccc5-c5ccc6ccccc6c54)c3)cc21 ; CC1(C)c2ccccc2Oc2cc(-c3cccc(C4(c5ccccc5)c5ccccc5-c5ccc6ccccc6c54)c3)ccc21]

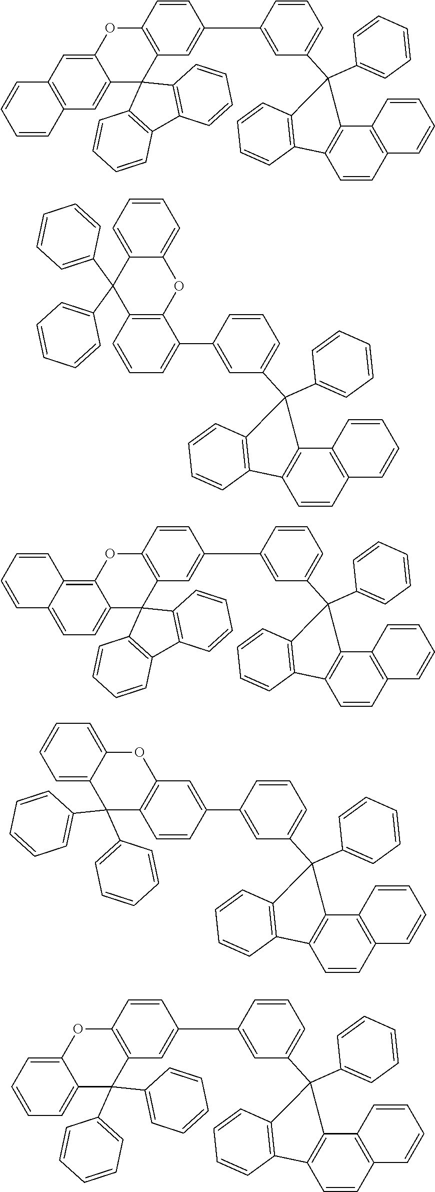

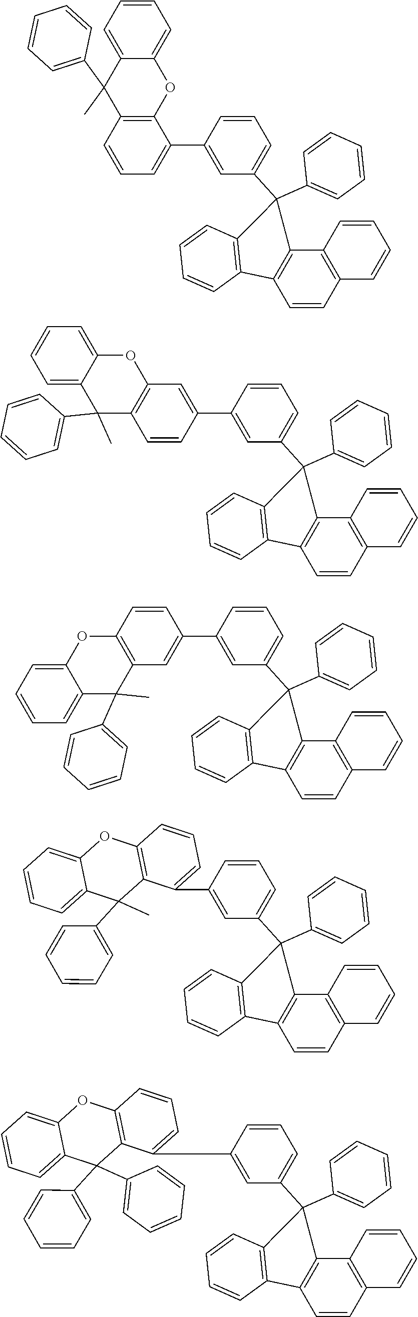 [SMILES: CC1(c2ccccc2)c2ccccc2Oc2c(-c3cccc(C4(c5ccccc5)c5ccccc5-c5ccc6ccccc6c54)c3)cccc21 ; CC1(c2ccccc2)c2ccccc2Oc2cc(-c3cccc(C4(c5ccccc5)c5ccccc5-c5ccc6ccccc6c54)c3)ccc21 ; CC1(c2ccccc2)c2ccccc2Oc2ccc(-c3cccc(C4(c5ccccc5)c5ccccc5-c5ccc6ccccc6c54)c3)cc21 ; CC1(c2ccccc2)c2ccccc2Oc2cccc(-c3cccc(C4(c5ccccc5)c5ccccc5-c5ccc6ccccc6c54)c3)c21 ; c1ccc(C2(c3ccccc3)c3ccccc3Oc3cccc(-c4cccc(C5(c6ccccc6)c6ccccc6-c6ccc7ccccc7c65)c4)c32)cc1]

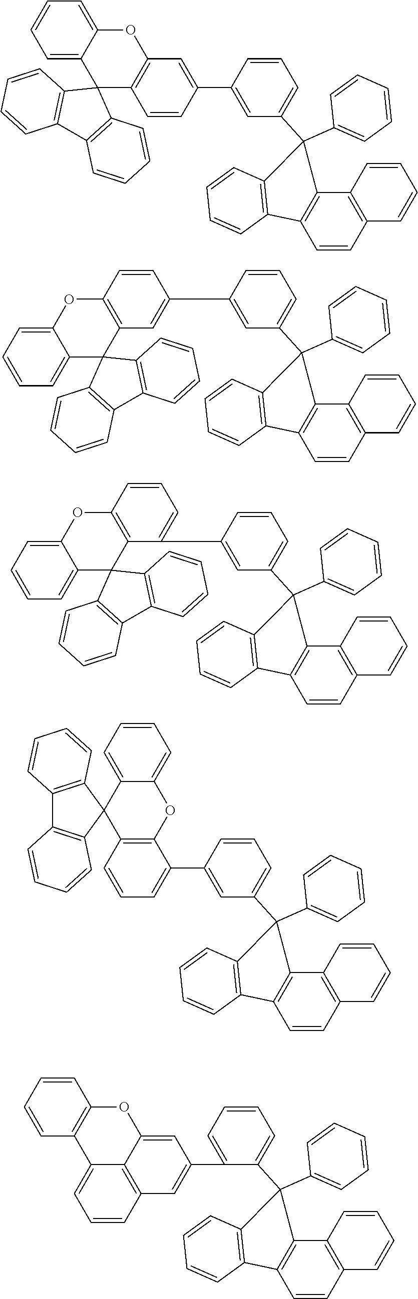

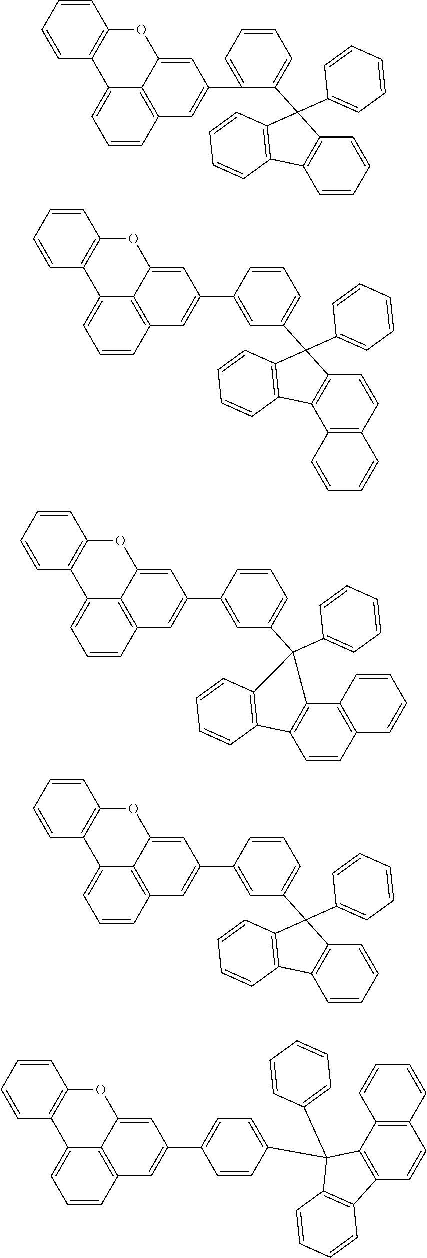 [SMILES: c1ccc(C2(c3ccccc3-c3cc4c5c(cccc5c3)-c3ccccc3O4)c3ccccc3-c3ccccc32)cc1 ; c1ccc(C2(c3cccc(-c4cc5c6c(cccc6c4)-c4ccccc4O5)c3)c3ccccc3-c3c2ccc2ccccc32)cc1 ; c1ccc(C2(c3cccc(-c4cc5c6c(cccc6c4)-c4ccccc4O5)c3)c3ccccc3-c3ccc4ccccc4c32)cc1 ; c1ccc(C2(c3cccc(-c4cc5c6c(cccc6c4)-c4ccccc4O5)c3)c3ccccc3-c3ccccc32)cc1 ; c1ccc(C2(c3ccc(-c4cc5c6c(cccc6c4)-c4ccccc4O5)cc3)c3ccccc3-c3ccc4ccccc4c32)cc1]

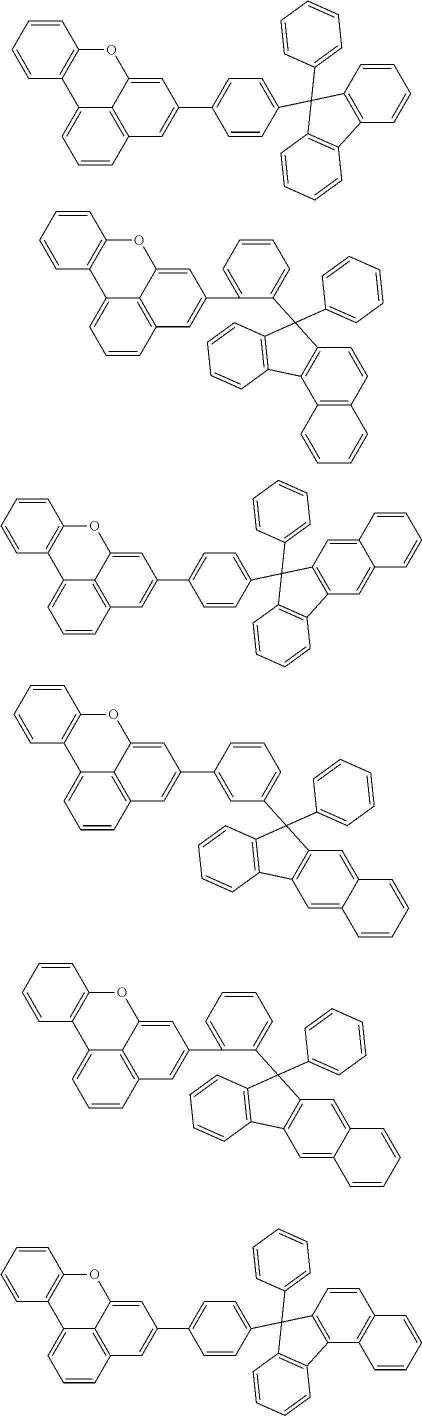

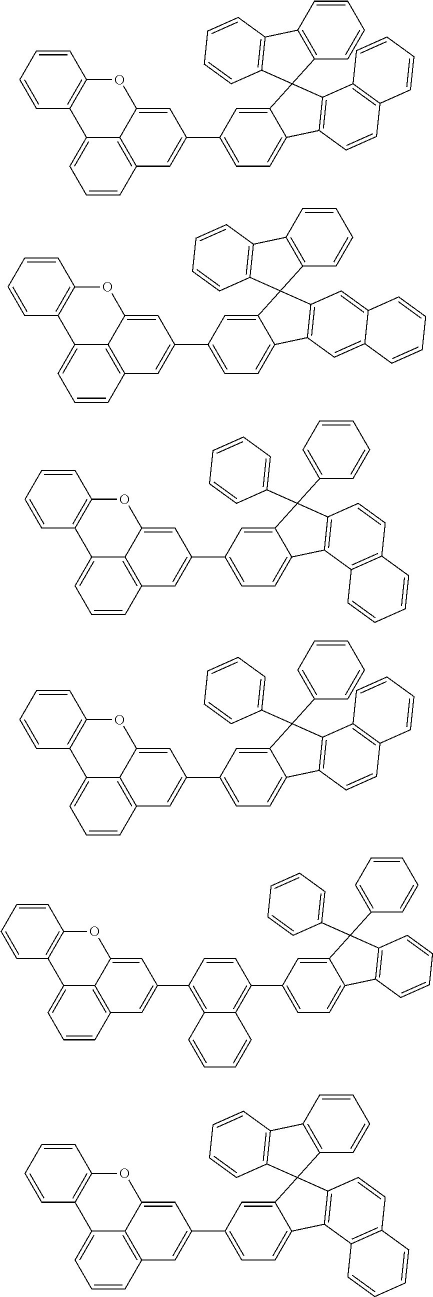

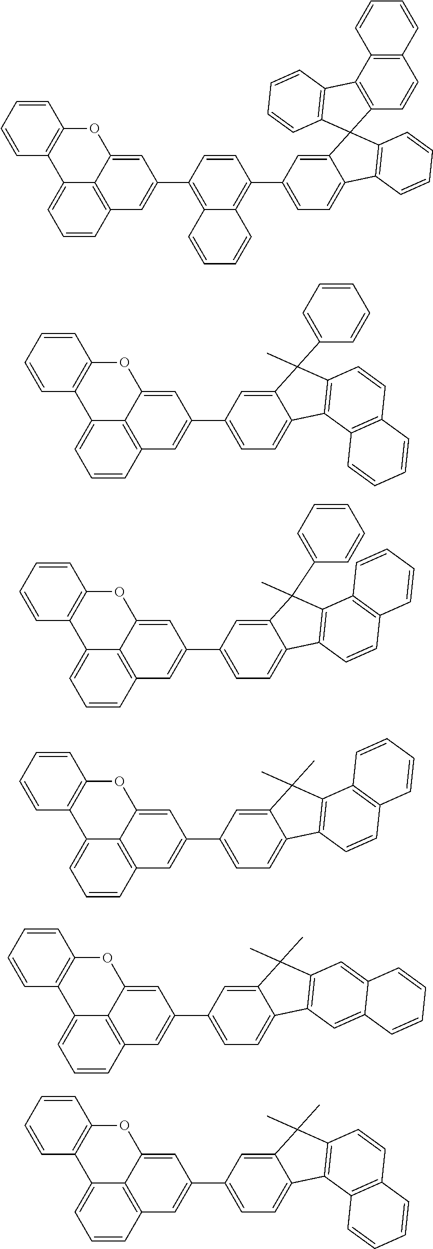

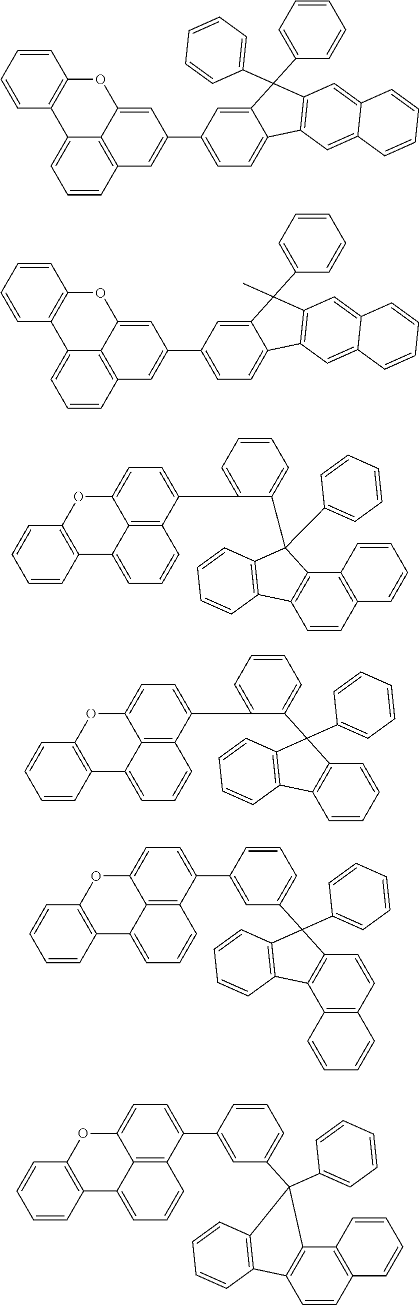

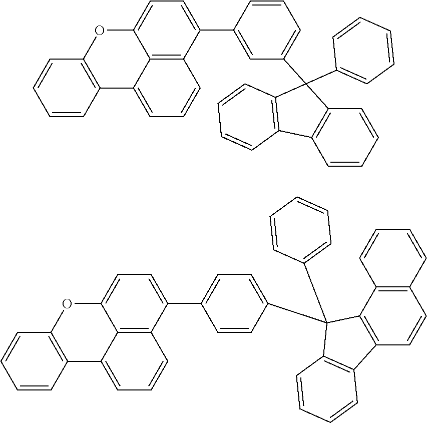

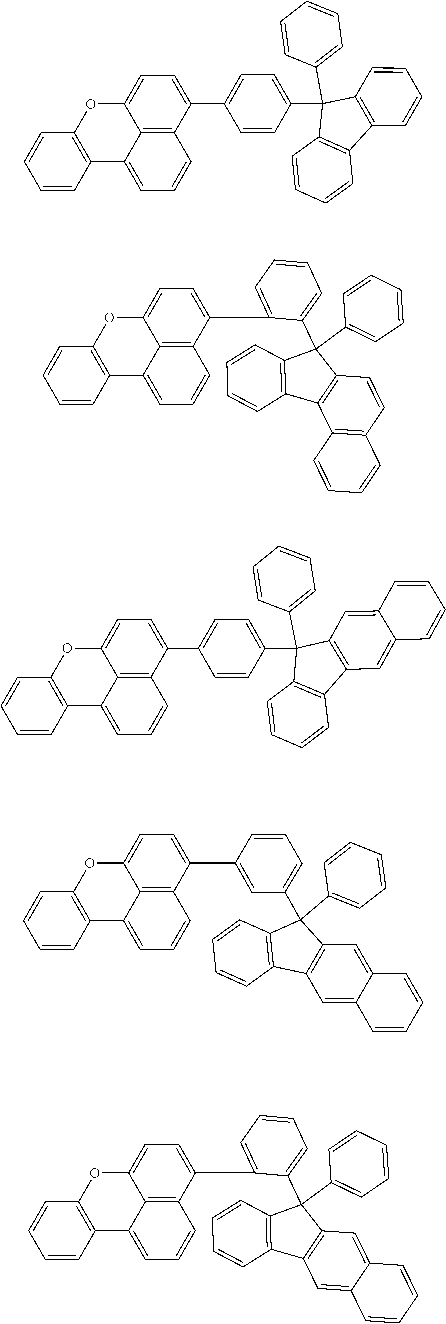 [SMILES: c1ccc(C2(c3ccc(-c4ccc5c6c(cccc46)-c4ccccc4O5)cc3)c3ccccc3-c3ccccc32)cc1 ; c1ccc(C2(c3ccccc3-c3ccc4c5c(cccc35)-c3ccccc3O4)c3ccccc3-c3c2ccc2ccccc32)cc1 ; c1ccc(C2(c3ccc(-c4ccc5c6c(cccc46)-c4ccccc4O5)cc3)c3ccccc3-c3cc4ccccc4cc32)cc1 ; c1ccc(C2(c3cccc(-c4ccc5c6c(cccc46)-c4ccccc4O5)c3)c3ccccc3-c3cc4ccccc4cc32)cc1 ; c1ccc(C2(c3ccccc3-c3ccc4c5c(cccc35)-c3ccccc3O4)c3ccccc3-c3cc4ccccc4cc32)cc1]

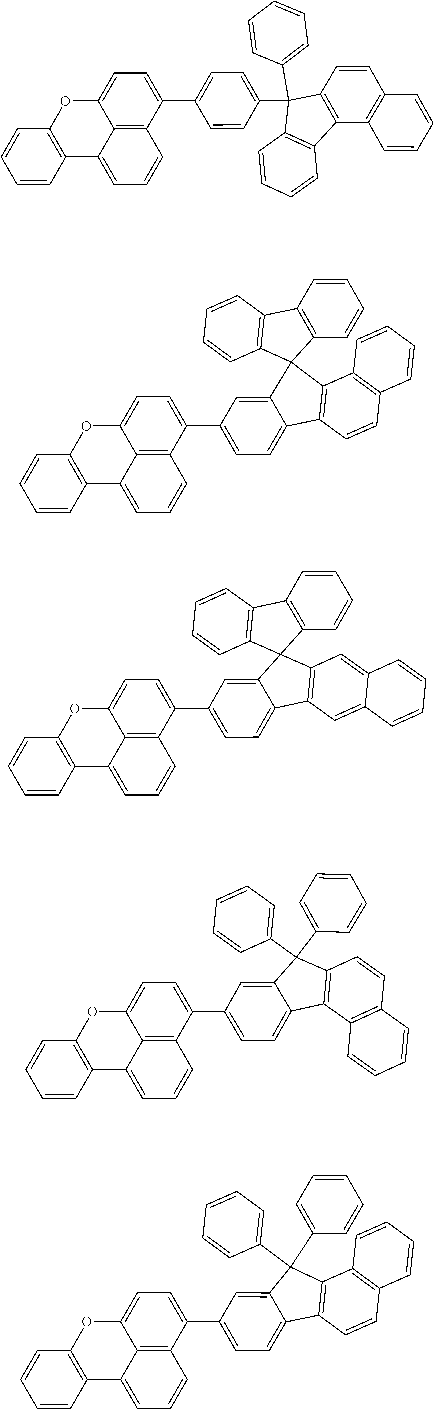 [SMILES: c1ccc(C2(c3ccc(-c4ccc5c6c(cccc46)-c4ccccc4O5)cc3)c3ccccc3-c3c2ccc2ccccc32)cc1 ; c1ccc2c(c1)Oc1ccc(-c3ccc4c(c3)C3(c5ccccc5-c5ccccc53)c3c-4ccc4ccccc34)c3cccc-2c13 ; c1ccc2c(c1)Oc1ccc(-c3ccc4c(c3)C3(c5ccccc5-c5ccccc53)c3cc5ccccc5cc3-4)c3cccc-2c13 ; c1ccc(C2(c3ccccc3)c3cc(-c4ccc5c6c(cccc46)-c4ccccc4O5)ccc3-c3c2ccc2ccccc32)cc1 ; c1ccc(C2(c3ccccc3)c3cc(-c4ccc5c6c(cccc46)-c4ccccc4O5)ccc3-c3ccc4ccccc4c32)cc1]

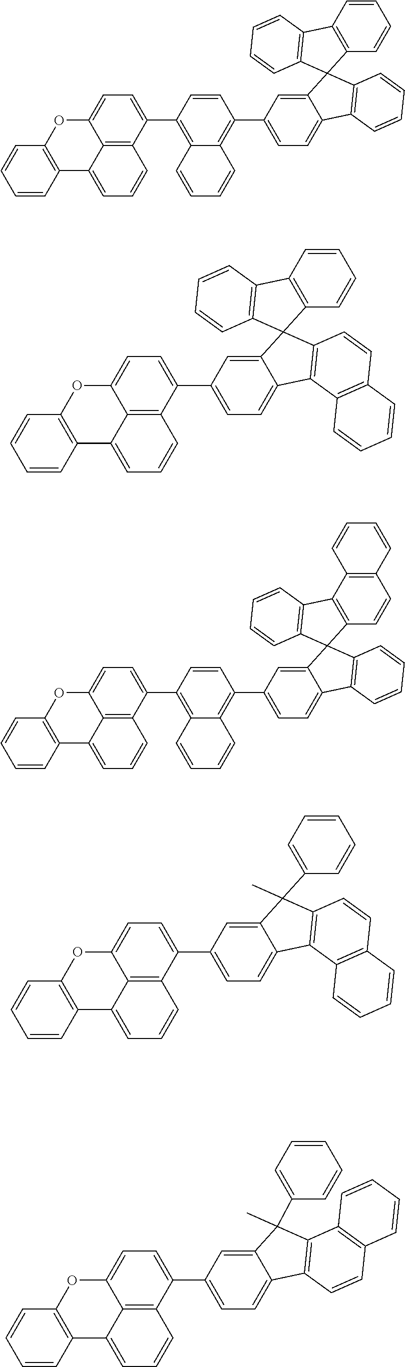 [SMILES: c1ccc2c(c1)Oc1ccc(-c3ccc(-c4ccc5c(c4)C4(c6ccccc6-c6ccccc64)c4ccccc4-5)c4ccccc34)c3cccc-2c13 ; c1ccc2c(c1)Oc1ccc(-c3ccc4c(c3)C3(c5ccccc5-c5ccccc53)c3ccc5ccccc5c3-4)c3cccc-2c13 ; c1ccc2c(c1)Oc1ccc(-c3ccc(-c4ccc5c(c4)C4(c6ccccc6-5)c5ccccc5-c5c4ccc4ccccc54)c4ccccc34)c3cccc-2c13 ; CC1(c2ccccc2)c2cc(-c3ccc4c5c(cccc35)-c3ccccc3O4)ccc2-c2c1ccc1ccccc21 ; CC1(c2ccccc2)c2cc(-c3ccc4c5c(cccc35)-c3ccccc3O4)ccc2-c2ccc3ccccc3c21]

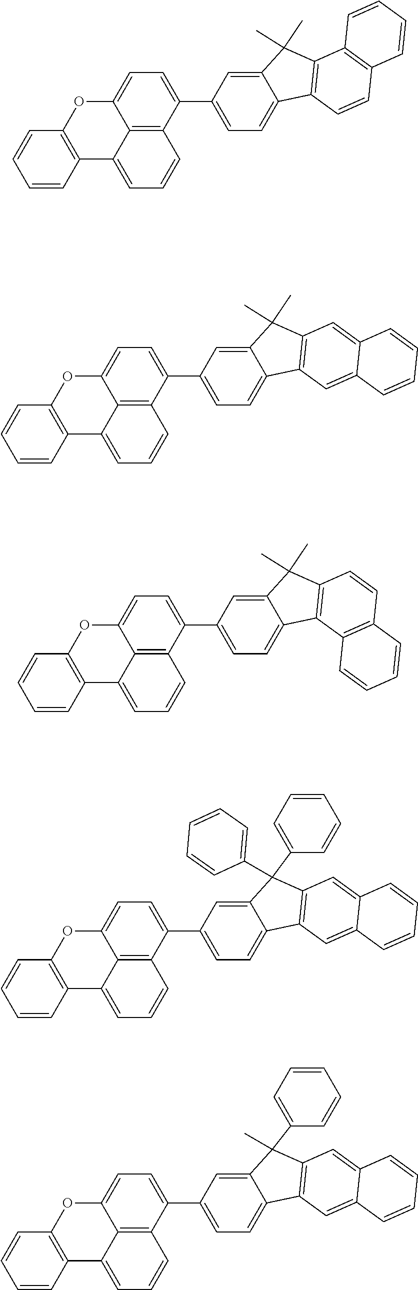 [SMILES: CC1(C)c2cc(-c3ccc4c5c(cccc35)-c3ccccc3O4)ccc2-c2ccc3ccccc3c21 ; CC1(C)c2cc(-c3ccc4c5c(cccc35)-c3ccccc3O4)ccc2-c2cc3ccccc3cc21 ; CC1(C)c2cc(-c3ccc4c5c(cccc35)-c3ccccc3O4)ccc2-c2c1ccc1ccccc21 ; c1ccc(C2(c3ccccc3)c3cc(-c4ccc5c6c(cccc46)-c4ccccc4O5)ccc3-c3cc4ccccc4cc32)cc1 ; CC1(c2ccccc2)c2cc(-c3ccc4c5c(cccc35)-c3ccccc3O4)ccc2-c2cc3ccccc3cc21]

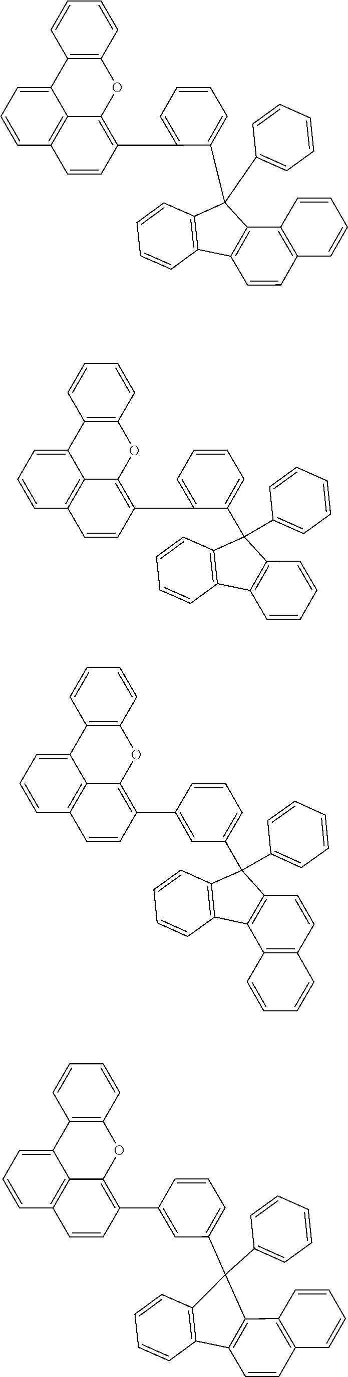 [SMILES: c1ccc(C2(c3ccccc3-c3ccc4cccc5c4c3Oc3ccccc3-5)c3ccccc3-c3ccc4ccccc4c32)cc1 ; c1ccc(C2(c3ccccc3-c3ccc4cccc5c4c3Oc3ccccc3-5)c3ccccc3-c3ccccc32)cc1 ; c1ccc(C2(c3cccc(-c4ccc5cccc6c5c4Oc4ccccc4-6)c3)c3ccccc3-c3c2ccc2ccccc32)cc1 ; c1ccc(C2(c3cccc(-c4ccc5cccc6c5c4Oc4ccccc4-6)c3)c3ccccc3-c3ccc4ccccc4c32)cc1]

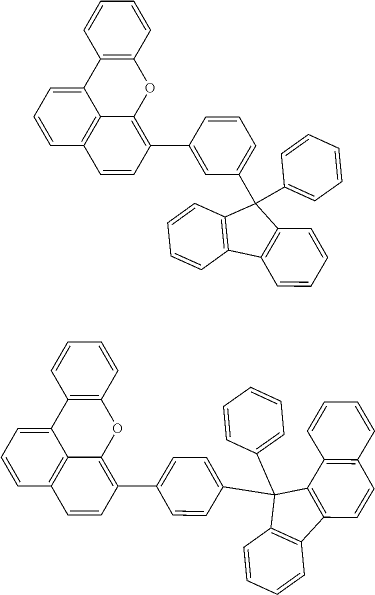 [SMILES: c1ccc(C2(c3cccc(-c4ccc5cccc6c5c4Oc4ccccc4-6)c3)c3ccccc3-c3ccccc32)cc1 ; c1ccc(C2(c3ccc(-c4ccc5cccc6c5c4Oc4ccccc4-6)cc3)c3ccccc3-c3ccc4ccccc4c32)cc1]

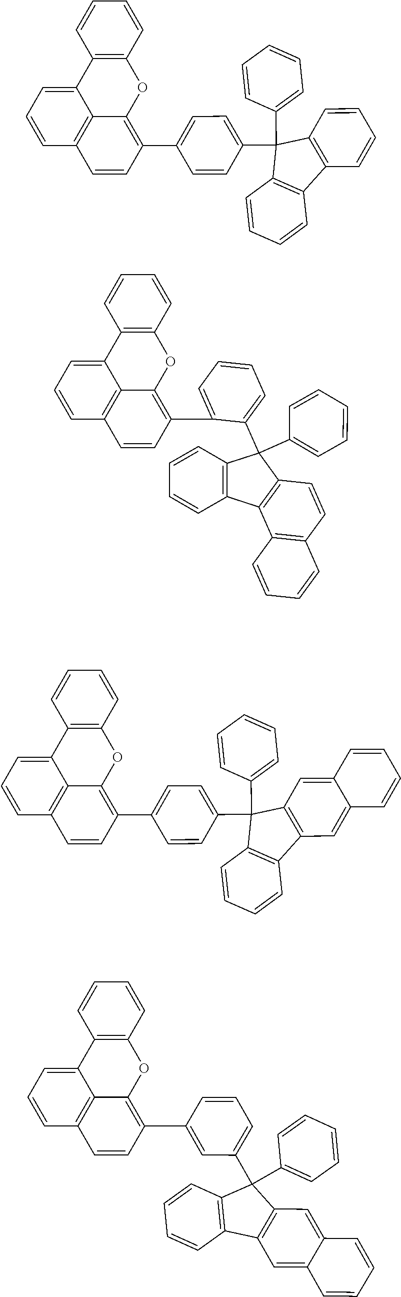 [SMILES: c1ccc(C2(c3ccc(-c4ccc5cccc6c5c4Oc4ccccc4-6)cc3)c3ccccc3-c3ccccc32)cc1 ; c1ccc(C2(c3ccccc3-c3ccc4cccc5c4c3Oc3ccccc3-5)c3ccccc3-c3c2ccc2ccccc32)cc1 ; c1ccc(C2(c3ccc(-c4ccc5cccc6c5c4Oc4ccccc4-6)cc3)c3ccccc3-c3cc4ccccc4cc32)cc1 ; c1ccc(C2(c3cccc(-c4ccc5cccc6c5c4Oc4ccccc4-6)c3)c3ccccc3-c3cc4ccccc4cc32)cc1]

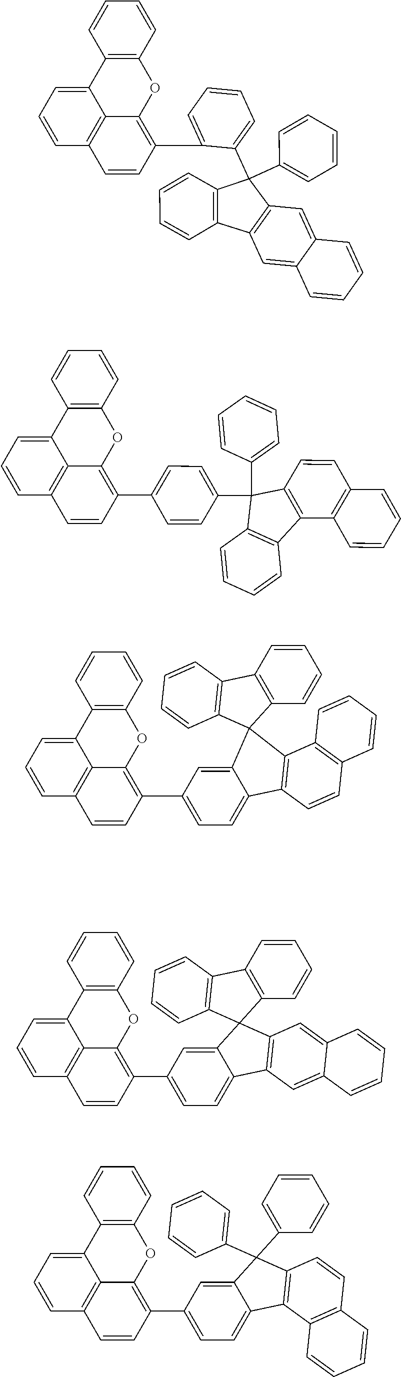 [SMILES: c1ccc(C2(c3ccccc3-c3ccc4cccc5c4c3Oc3ccccc3-5)c3ccccc3-c3cc4ccccc4cc32)cc1 ; c1ccc(C2(c3ccc(-c4ccc5cccc6c5c4Oc4ccccc4-6)cc3)c3ccccc3-c3c2ccc2ccccc32)cc1 ; c1ccc2c(c1)Oc1c(-c3ccc4c(c3)C3(c5ccccc5-c5ccccc53)c3c-4ccc4ccccc34)ccc3cccc-2c13 ; c1ccc2c(c1)Oc1c(-c3ccc4c(c3)C3(c5ccccc5-c5ccccc53)c3cc5ccccc5cc3-4)ccc3cccc-2c13 ; c1ccc(C2(c3ccccc3)c3cc(-c4ccc5cccc6c5c4Oc4ccccc4-6)ccc3-c3c2ccc2ccccc32)cc1]

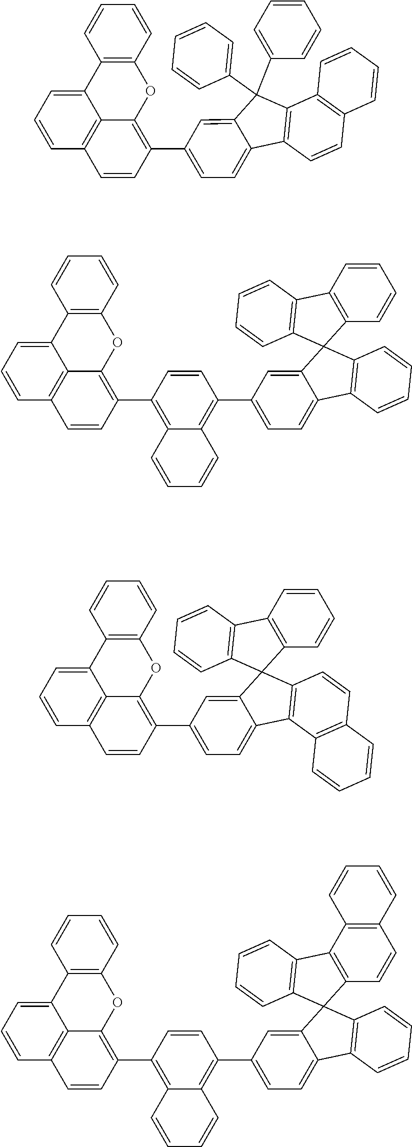 [SMILES: c1ccc(C2(c3ccccc3)c3cc(-c4ccc5cccc6c5c4Oc4ccccc4-6)ccc3-c3ccc4ccccc4c32)cc1 ; c1ccc2c(c1)Oc1c(-c3ccc(-c4ccc5c(c4)C4(c6ccccc6-c6ccccc64)c4ccccc4-5)c4ccccc34)ccc3cccc-2c13 ; c1ccc2c(c1)Oc1c(-c3ccc4c(c3)C3(c5ccccc5-c5ccccc53)c3ccc5ccccc5c3-4)ccc3cccc-2c13 ; c1ccc2c(c1)Oc1c(-c3ccc(-c4ccc5c(c4)C4(c6ccccc6-5)c5ccccc5-c5c4ccc4ccccc54)c4ccccc34)ccc3cccc-2c13]

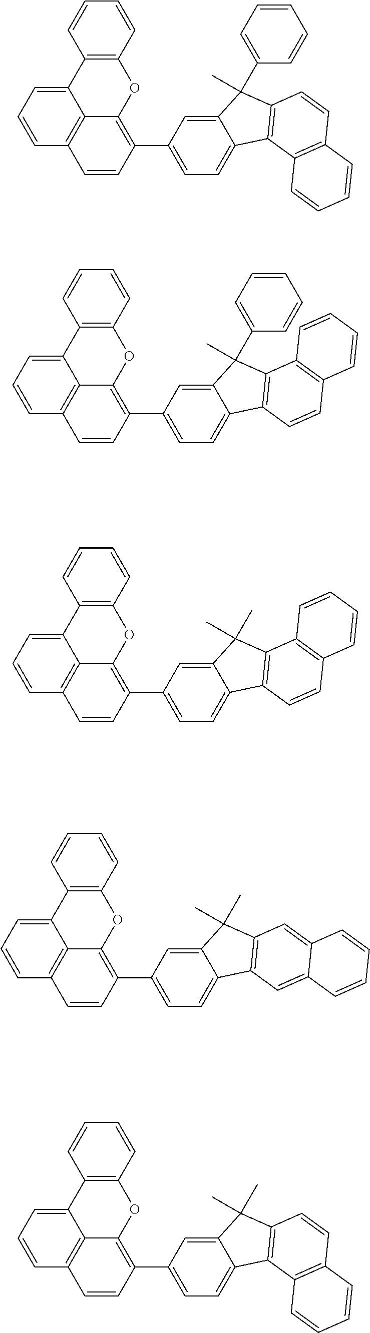 [SMILES: CC1(c2ccccc2)c2cc(-c3ccc4cccc5c4c3Oc3ccccc3-5)ccc2-c2c1ccc1ccccc21 ; CC1(c2ccccc2)c2cc(-c3ccc4cccc5c4c3Oc3ccccc3-5)ccc2-c2ccc3ccccc3c21 ; CC1(C)c2cc(-c3ccc4cccc5c4c3Oc3ccccc3-5)ccc2-c2ccc3ccccc3c21 ; CC1(C)c2cc(-c3ccc4cccc5c4c3Oc3ccccc3-5)ccc2-c2cc3ccccc3cc21 ; CC1(C)c2cc(-c3ccc4cccc5c4c3Oc3ccccc3-5)ccc2-c2c1ccc1ccccc21]

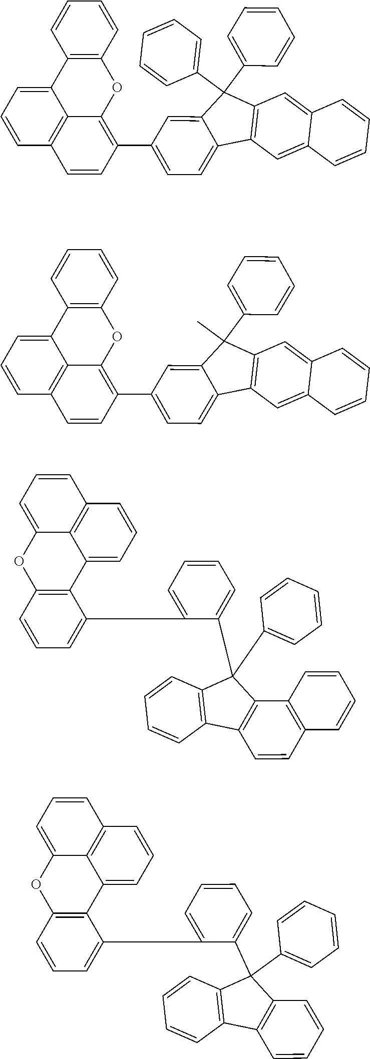 [SMILES: c1ccc(C2(c3ccccc3)c3cc(-c4ccc5cccc6c5c4Oc4ccccc4-6)ccc3-c3cc4ccccc4cc32)cc1 ; CC1(c2ccccc2)c2cc(-c3ccc4cccc5c4c3Oc3ccccc3-5)ccc2-c2cc3ccccc3cc21 ; c1ccc(C2(c3ccccc3-c3cccc4c3-c3cccc5cccc(c35)O4)c3ccccc3-c3ccc4ccccc4c32)cc1 ; c1ccc(C2(c3ccccc3-c3cccc4c3-c3cccc5cccc(c35)O4)c3ccccc3-c3ccccc32)cc1]

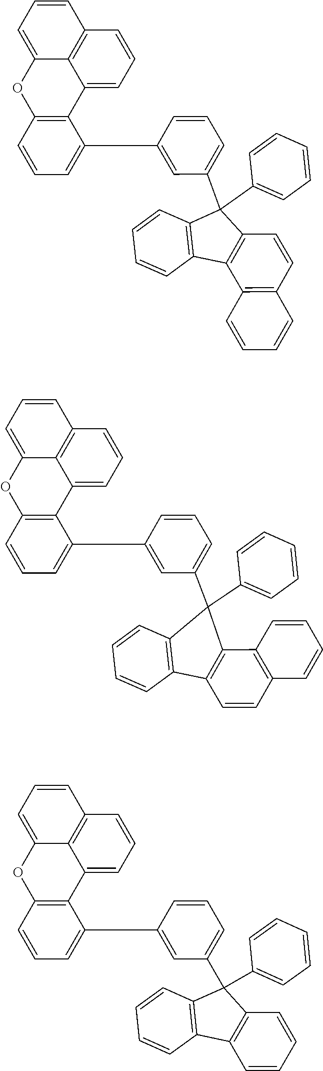 [SMILES: c1ccc(C2(c3cccc(-c4cccc5c4-c4cccc6cccc(c46)O5)c3)c3ccccc3-c3c2ccc2ccccc32)cc1 ; c1ccc(C2(c3cccc(-c4cccc5c4-c4cccc6cccc(c46)O5)c3)c3ccccc3-c3ccc4ccccc4c32)cc1 ; c1ccc(C2(c3cccc(-c4cccc5c4-c4cccc6cccc(c46)O5)c3)c3ccccc3-c3ccccc32)cc1]

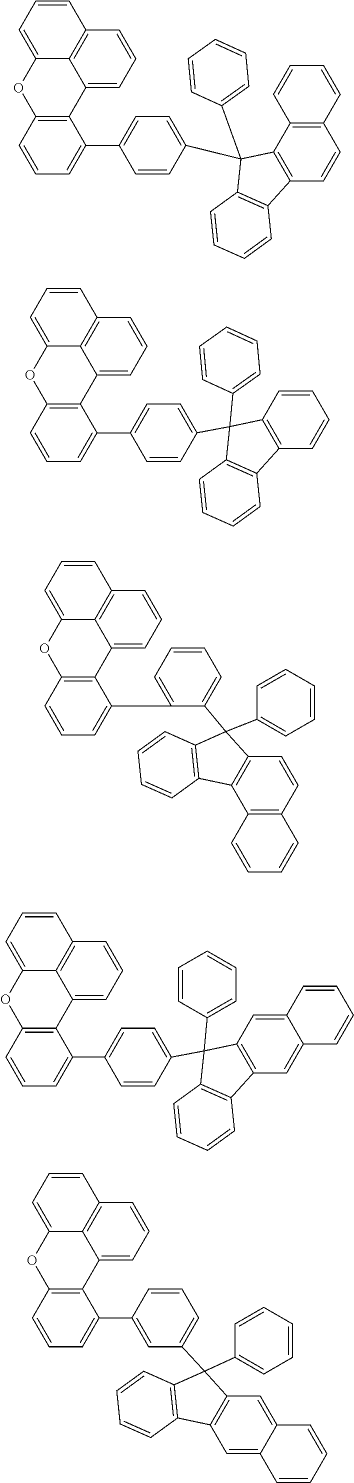 [SMILES: c1ccc(C2(c3ccc(-c4cccc5c4-c4cccc6cccc(c46)O5)cc3)c3ccccc3-c3ccc4ccccc4c32)cc1 ; c1ccc(C2(c3ccc(-c4cccc5c4-c4cccc6cccc(c46)O5)cc3)c3ccccc3-c3ccccc32)cc1 ; c1ccc(C2(c3ccccc3-c3cccc4c3-c3cccc5cccc(c35)O4)c3ccccc3-c3c2ccc2ccccc32)cc1 ; c1ccc(C2(c3ccc(-c4cccc5c4-c4cccc6cccc(c46)O5)cc3)c3ccccc3-c3cc4ccccc4cc32)cc1 ; c1ccc(C2(c3cccc(-c4cccc5c4-c4cccc6cccc(c46)O5)c3)c3ccccc3-c3cc4ccccc4cc32)cc1]

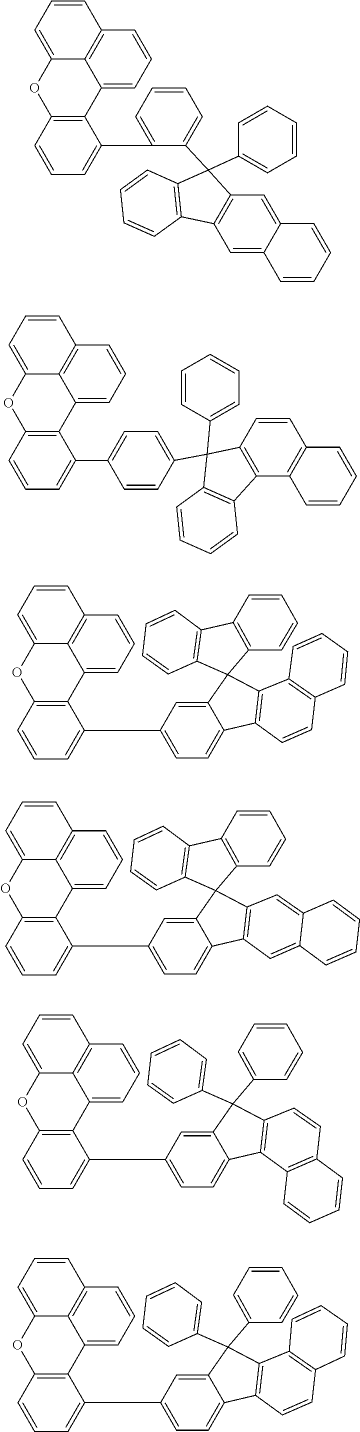

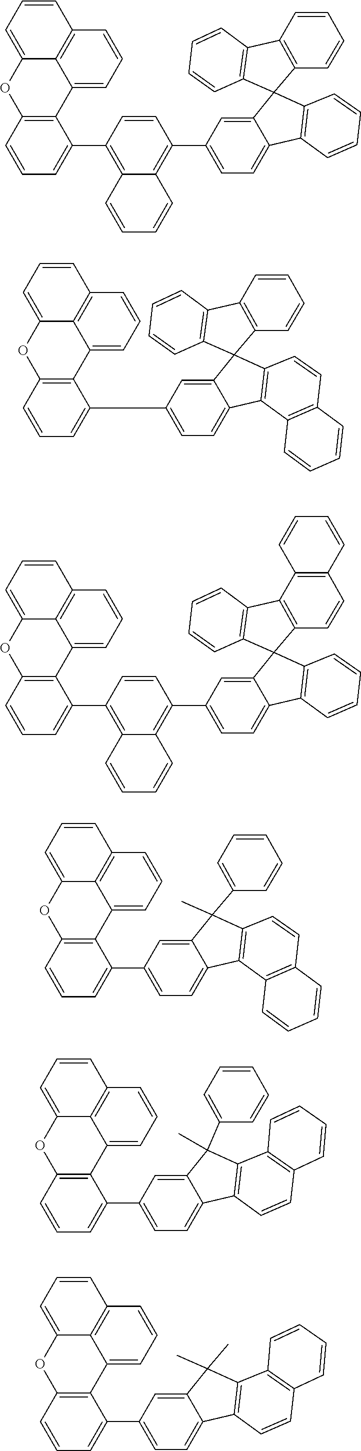

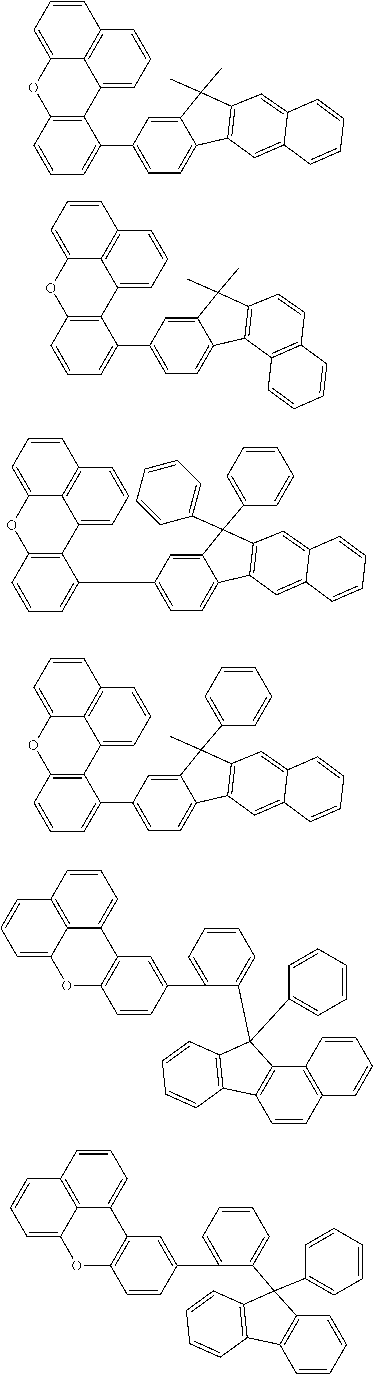

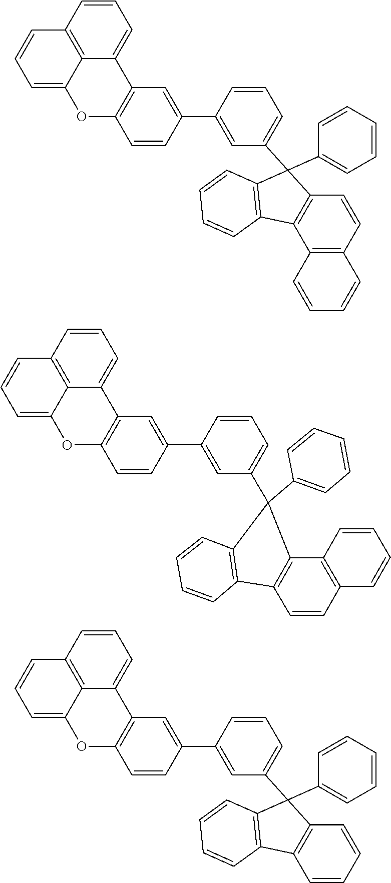 [SMILES: c1ccc(C2(c3cccc(-c4ccc5c(c4)-c4cccc6cccc(c46)O5)c3)c3ccccc3-c3c2ccc2ccccc32)cc1 ; c1ccc(C2(c3cccc(-c4ccc5c(c4)-c4cccc6cccc(c46)O5)c3)c3ccccc3-c3ccc4ccccc4c32)cc1 ; c1ccc(C2(c3cccc(-c4ccc5c(c4)-c4cccc6cccc(c46)O5)c3)c3ccccc3-c3ccccc32)cc1]

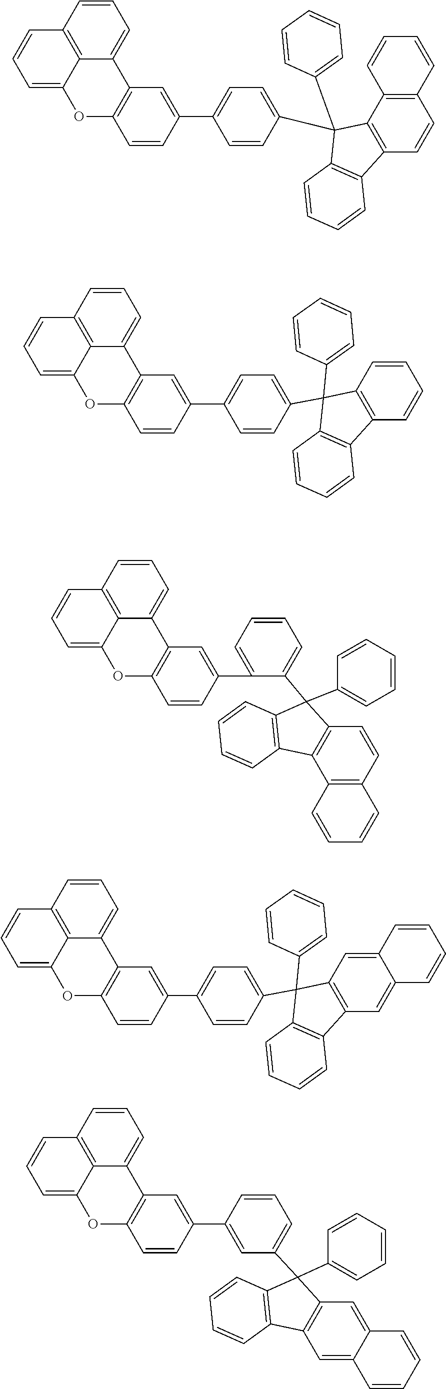 [SMILES: c1ccc(C2(c3ccc(-c4ccc5c(c4)-c4cccc6cccc(c46)O5)cc3)c3ccccc3-c3ccc4ccccc4c32)cc1 ; c1ccc(C2(c3ccc(-c4ccc5c(c4)-c4cccc6cccc(c46)O5)cc3)c3ccccc3-c3ccccc32)cc1 ; c1ccc(C2(c3ccccc3-c3ccc4c(c3)-c3cccc5cccc(c35)O4)c3ccccc3-c3c2ccc2ccccc32)cc1 ; c1ccc(C2(c3ccc(-c4ccc5c(c4)-c4cccc6cccc(c46)O5)cc3)c3ccccc3-c3cc4ccccc4cc32)cc1 ; c1ccc(C2(c3cccc(-c4ccc5c(c4)-c4cccc6cccc(c46)O5)c3)c3ccccc3-c3cc4ccccc4cc32)cc1]

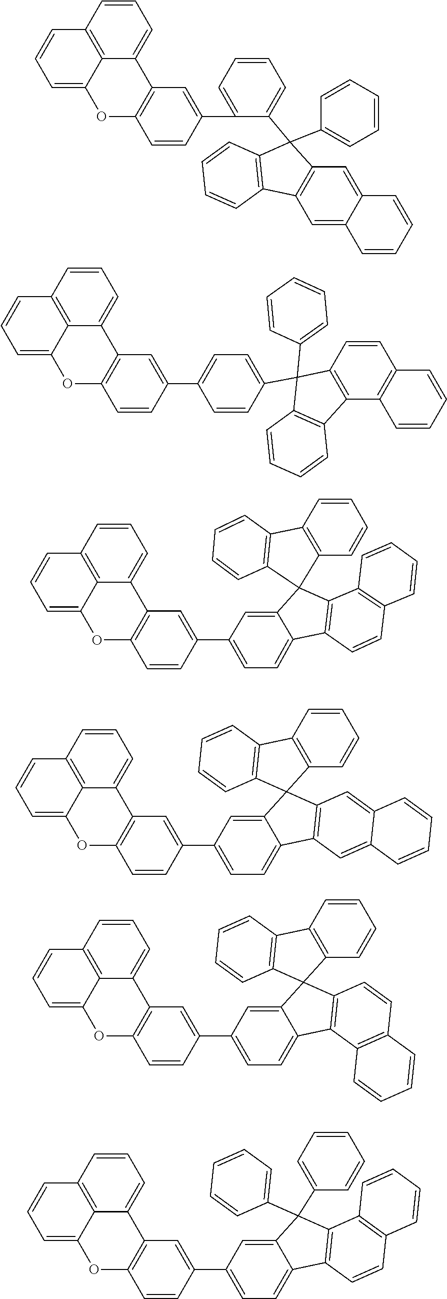

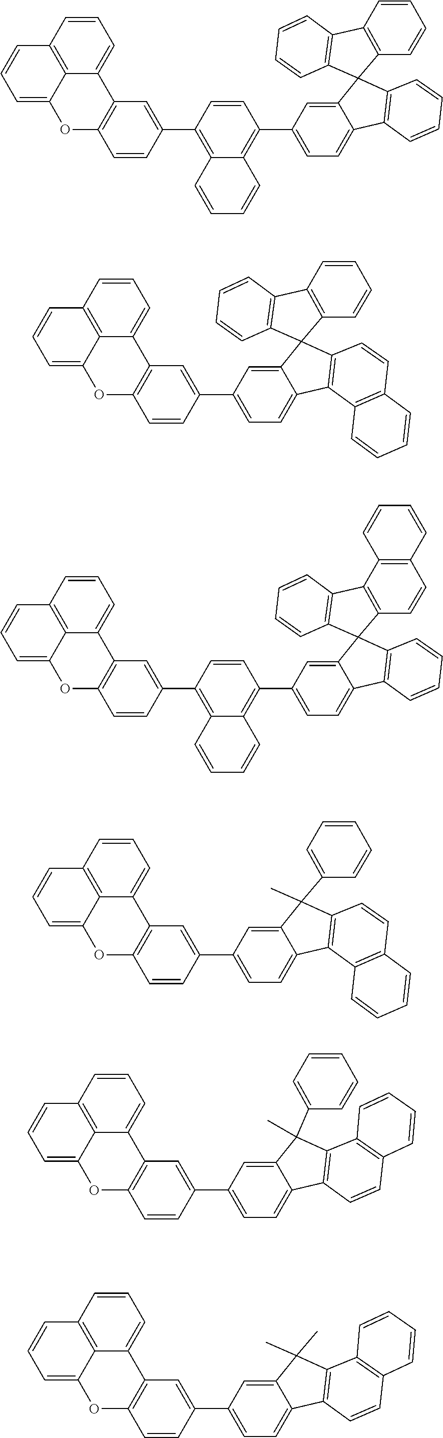 [SMILES: c1ccc2c(c1)-c1ccccc1C21c2ccccc2-c2ccc(-c3ccc(-c4ccc5c(c4)-c4cccc6cccc(c46)O5)c4ccccc34)cc21 ; c1ccc2c(c1)-c1ccccc1C21c2cc(-c3ccc4c(c3)-c3cccc5cccc(c35)O4)ccc2-c2c1ccc1ccccc21 ; c1ccc2c(c1)-c1ccc(-c3ccc(-c4ccc5c(c4)-c4cccc6cccc(c46)O5)c4ccccc34)cc1C21c2ccccc2-c2c1ccc1ccccc21 ; CC1(c2ccccc2)c2cc(-c3ccc4c(c3)-c3cccc5cccc(c35)O4)ccc2-c2c1ccc1ccccc21 ; CC1(c2ccccc2)c2cc(-c3ccc4c(c3)-c3cccc5cccc(c35)O4)ccc2-c2ccc3ccccc3c21 ; CC1(C)c2cc(-c3ccc4c(c3)-c3cccc5cccc(c35)O4)ccc2-c2ccc3ccccc3c21]

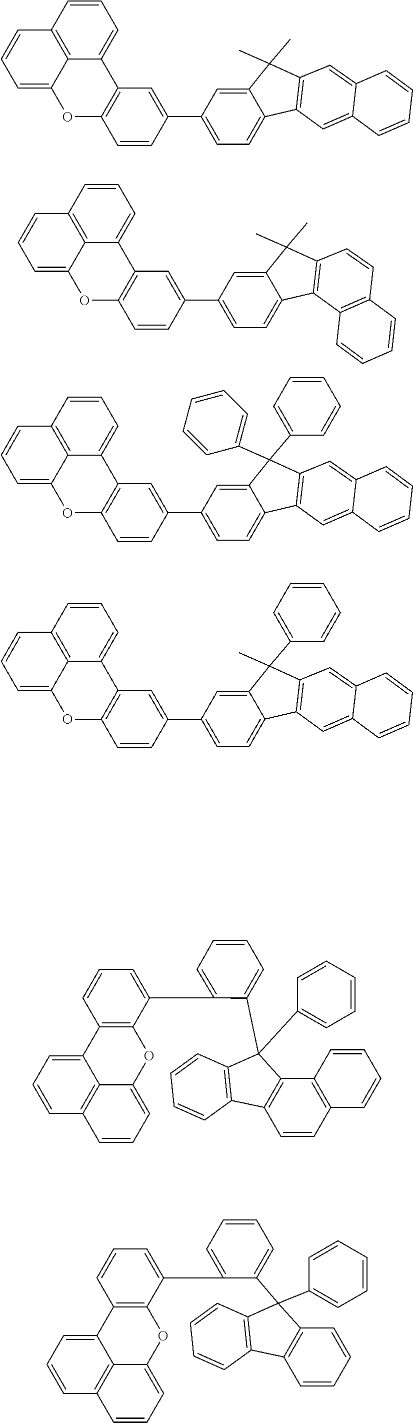

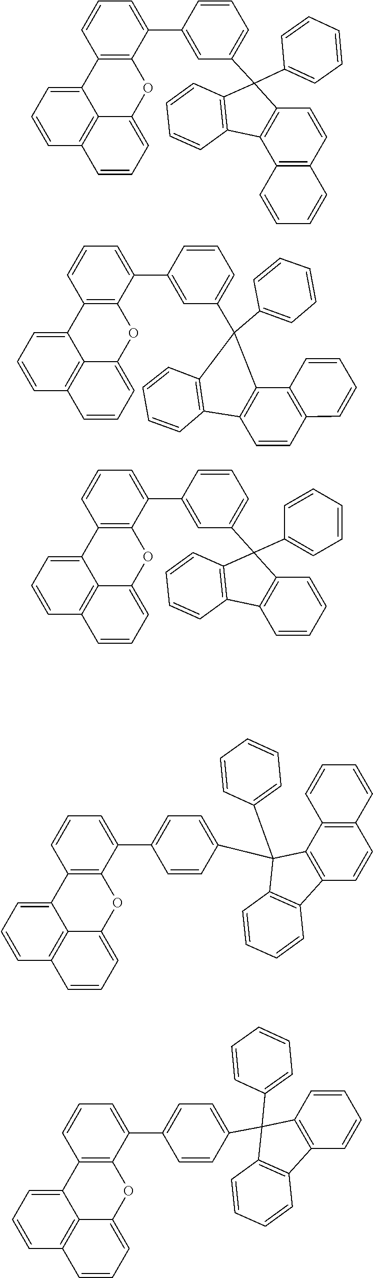

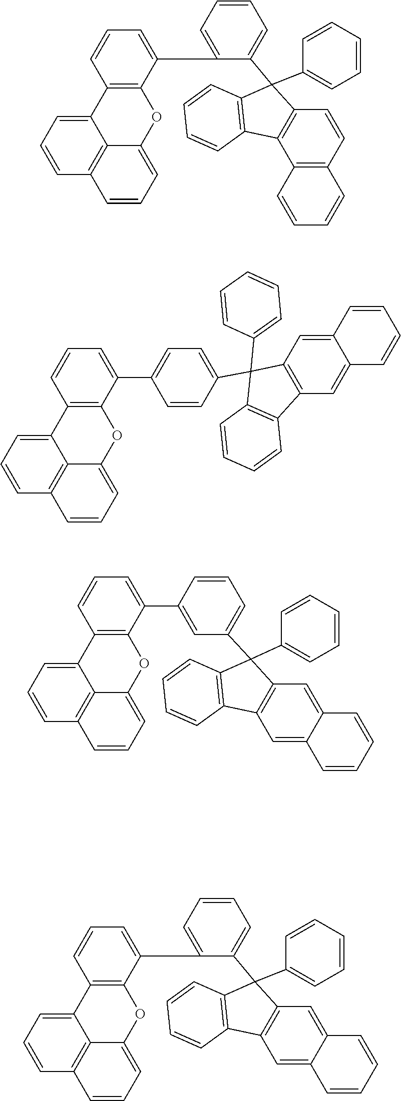 [SMILES: c1ccc(C2(c3ccccc3-c3cccc4c3Oc3cccc5cccc-4c35)c3ccccc3-c3c2ccc2ccccc32)cc1 ; c1ccc(C2(c3ccc(-c4cccc5c4Oc4cccc6cccc-5c46)cc3)c3ccccc3-c3cc4ccccc4cc32)cc1 ; c1ccc(C2(c3cccc(-c4cccc5c4Oc4cccc6cccc-5c46)c3)c3ccccc3-c3cc4ccccc4cc32)cc1 ; c1ccc(C2(c3ccccc3-c3cccc4c3Oc3cccc5cccc-4c35)c3ccccc3-c3cc4ccccc4cc32)cc1]

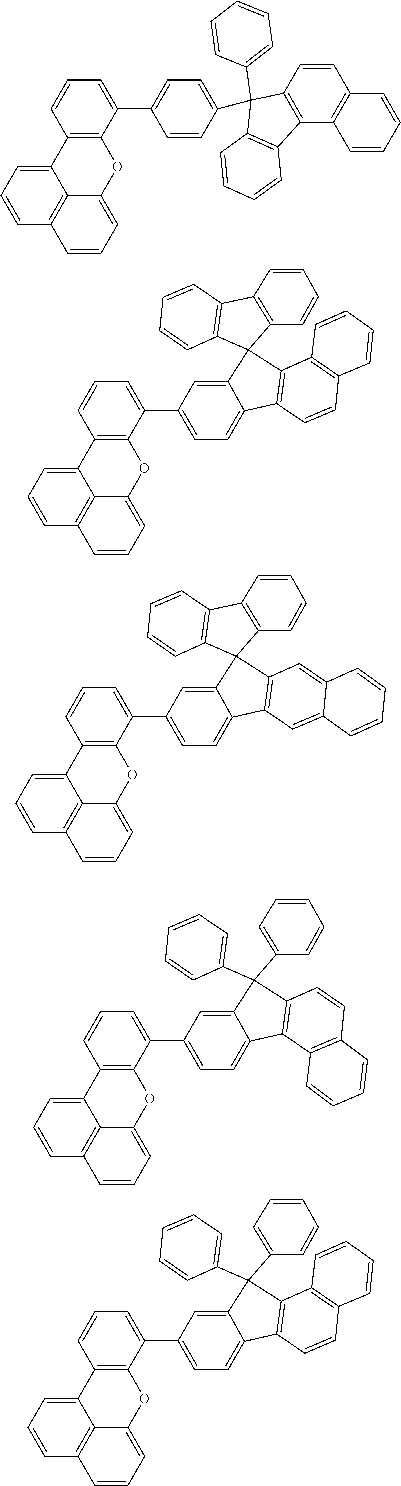 [SMILES: c1ccc(C2(c3ccc(-c4cccc5c4Oc4cccc6cccc-5c46)cc3)c3ccccc3-c3c2ccc2ccccc32)cc1 ; c1ccc2c(c1)-c1ccccc1C21c2cc(-c3cccc4c3Oc3cccc5cccc-4c35)ccc2-c2ccc3ccccc3c21 ; c1ccc2c(c1)-c1ccccc1C21c2cc(-c3cccc4c3Oc3cccc5cccc-4c35)ccc2-c2cc3ccccc3cc21 ; c1ccc(C2(c3ccccc3)c3cc(-c4cccc5c4Oc4cccc6cccc-5c46)ccc3-c3c2ccc2ccccc32)cc1 ; c1ccc(C2(c3ccccc3)c3cc(-c4cccc5c4Oc4cccc6cccc-5c46)ccc3-c3ccc4ccccc4c32)cc1]

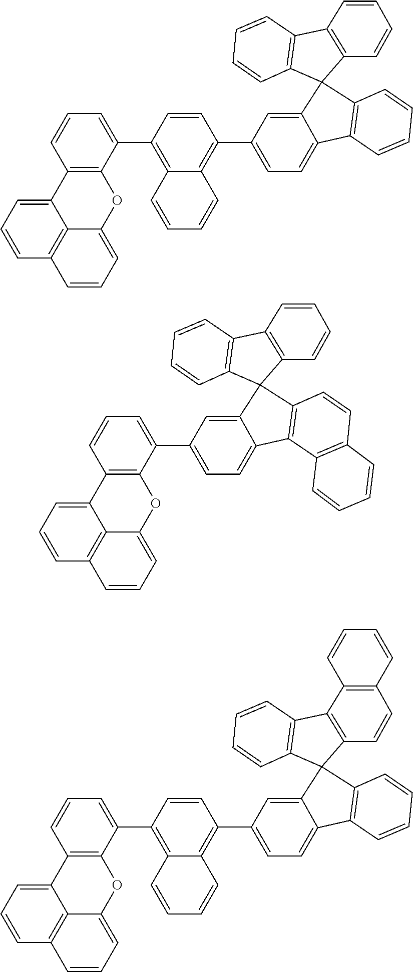 [SMILES: c1ccc2c(c1)-c1ccccc1C21c2ccccc2-c2ccc(-c3ccc(-c4cccc5c4Oc4cccc6cccc-5c46)c4ccccc34)cc21 ; c1ccc2c(c1)-c1ccccc1C21c2cc(-c3cccc4c3Oc3cccc5cccc-4c35)ccc2-c2c1ccc1ccccc21 ; c1ccc2c(c1)-c1ccc(-c3ccc(-c4cccc5c4Oc4cccc6cccc-5c46)c4ccccc34)cc1C21c2ccccc2-c2c1ccc1ccccc21]

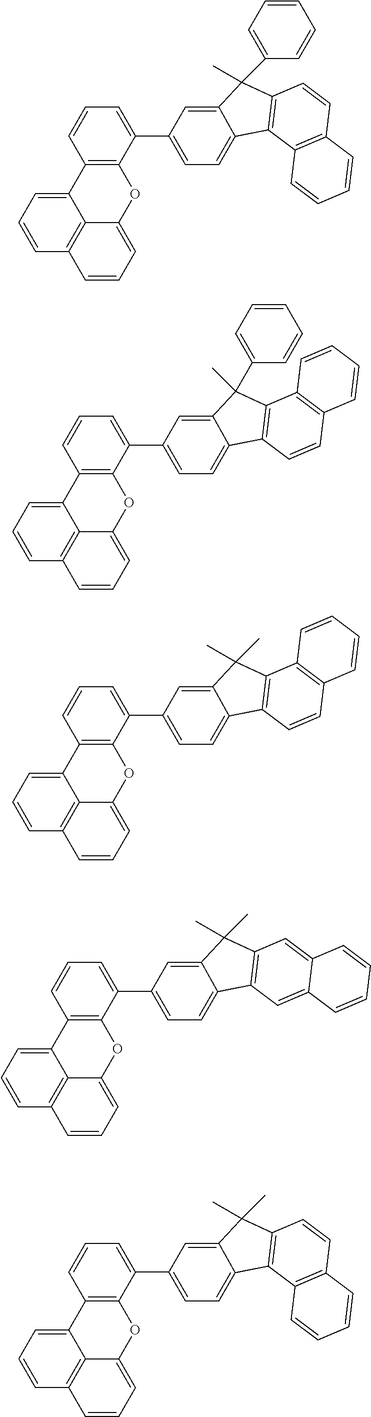 [SMILES: CC1(c2ccccc2)c2cc(-c3cccc4c3Oc3cccc5cccc-4c35)ccc2-c2c1ccc1ccccc21 ; CC1(c2ccccc2)c2cc(-c3cccc4c3Oc3cccc5cccc-4c35)ccc2-c2ccc3ccccc3c21 ; CC1(C)c2cc(-c3cccc4c3Oc3cccc5cccc-4c35)ccc2-c2ccc3ccccc3c21 ; CC1(C)c2cc(-c3cccc4c3Oc3cccc5cccc-4c35)ccc2-c2cc3ccccc3cc21 ; CC1(C)c2cc(-c3cccc4c3Oc3cccc5cccc-4c35)ccc2-c2c1ccc1ccccc21]

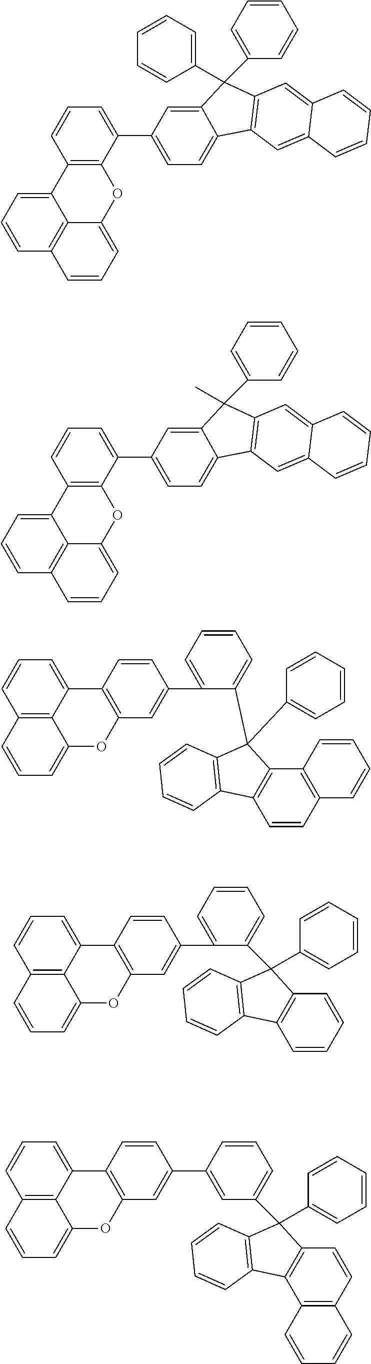 [SMILES: c1ccc(C2(c3ccccc3)c3cc(-c4cccc5c4Oc4cccc6cccc-5c46)ccc3-c3cc4ccccc4cc32)cc1 ; CC1(c2ccccc2)c2cc(-c3cccc4c3Oc3cccc5cccc-4c35)ccc2-c2cc3ccccc3cc21 ; c1ccc(C2(c3ccccc3-c3ccc4c(c3)Oc3cccc5cccc-4c35)c3ccccc3-c3ccc4ccccc4c32)cc1 ; c1ccc(C2(c3ccccc3-c3ccc4c(c3)Oc3cccc5cccc-4c35)c3ccccc3-c3ccccc32)cc1 ; c1ccc(C2(c3cccc(-c4ccc5c(c4)Oc4cccc6cccc-5c46)c3)c3ccccc3-c3c2ccc2ccccc32)cc1]

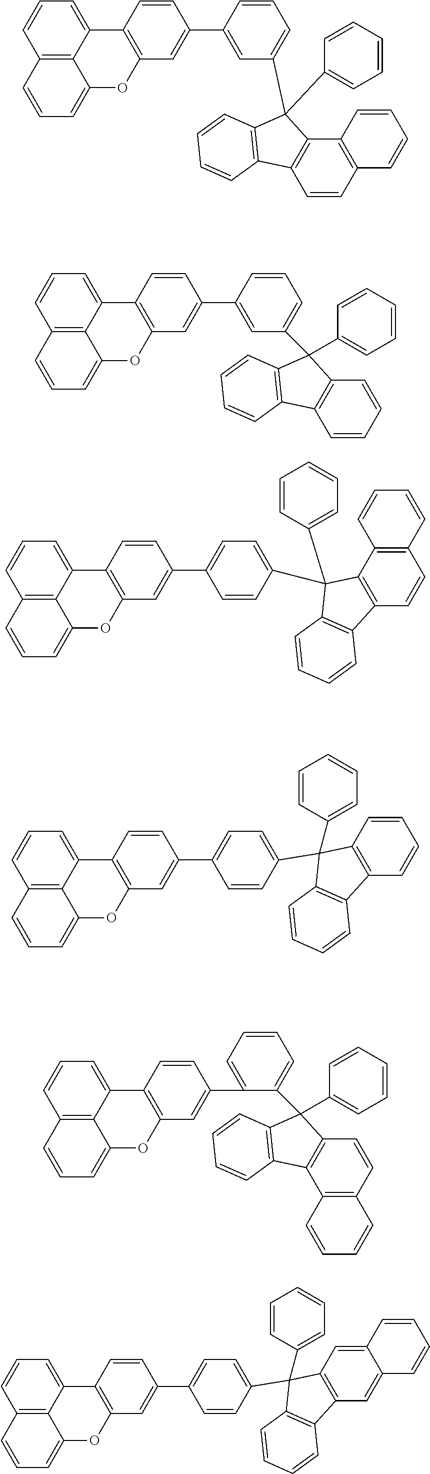

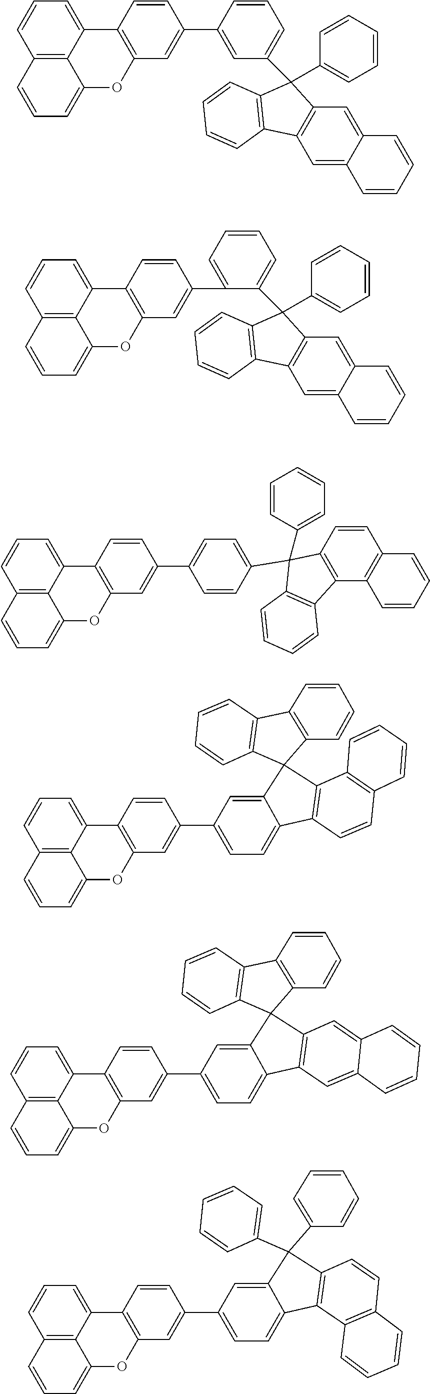

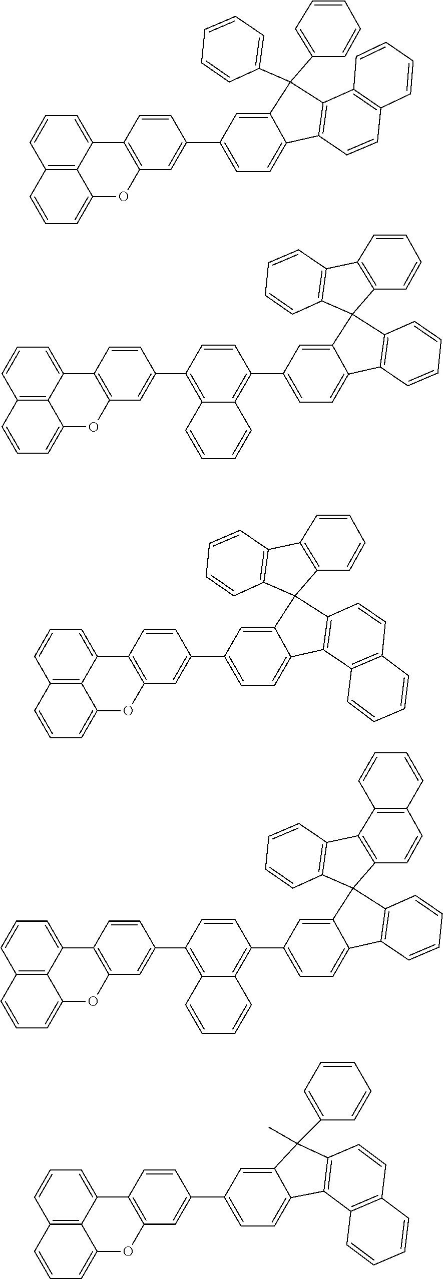

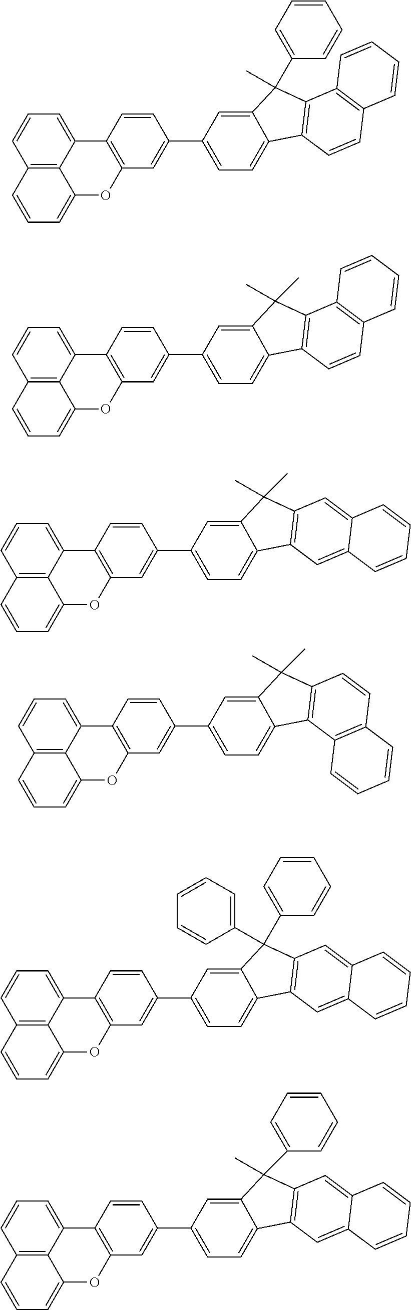 [SMILES: CC1(c2ccccc2)c2cc(-c3ccc4c(c3)Oc3cccc5cccc-4c35)ccc2-c2ccc3ccccc3c21 ; CC1(C)c2cc(-c3ccc4c(c3)Oc3cccc5cccc-4c35)ccc2-c2ccc3ccccc3c21 ; CC1(C)c2cc(-c3ccc4c(c3)Oc3cccc5cccc-4c35)ccc2-c2cc3ccccc3cc21 ; CC1(C)c2cc(-c3ccc4c(c3)Oc3cccc5cccc-4c35)ccc2-c2c1ccc1ccccc21 ; c1ccc(C2(c3ccccc3)c3cc(-c4ccc5c(c4)Oc4cccc6cccc-5c46)ccc3-c3cc4ccccc4cc32)cc1 ; CC1(c2ccccc2)c2cc(-c3ccc4c(c3)Oc3cccc5cccc-4c35)ccc2-c2cc3ccccc3cc21]

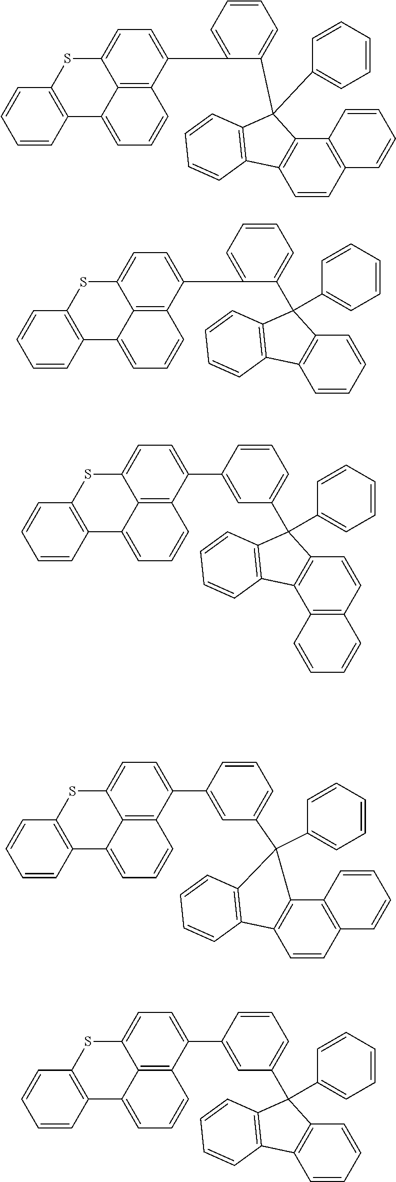

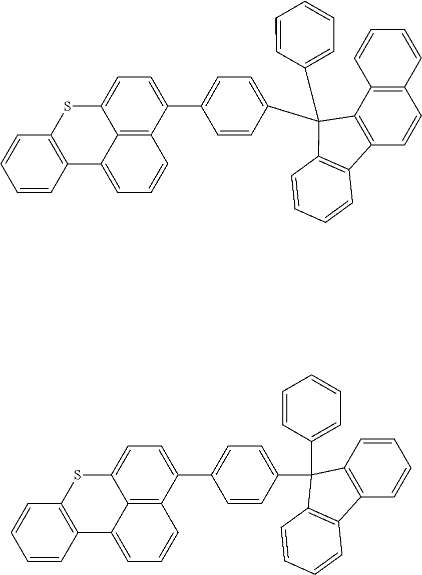 [SMILES: c1ccc(C2(c3ccc(-c4ccc5c6c(cccc46)-c4ccccc4S5)cc3)c3ccccc3-c3ccc4ccccc4c32)cc1 ; c1ccc(C2(c3ccc(-c4ccc5c6c(cccc46)-c4ccccc4S5)cc3)c3ccccc3-c3ccccc32)cc1]

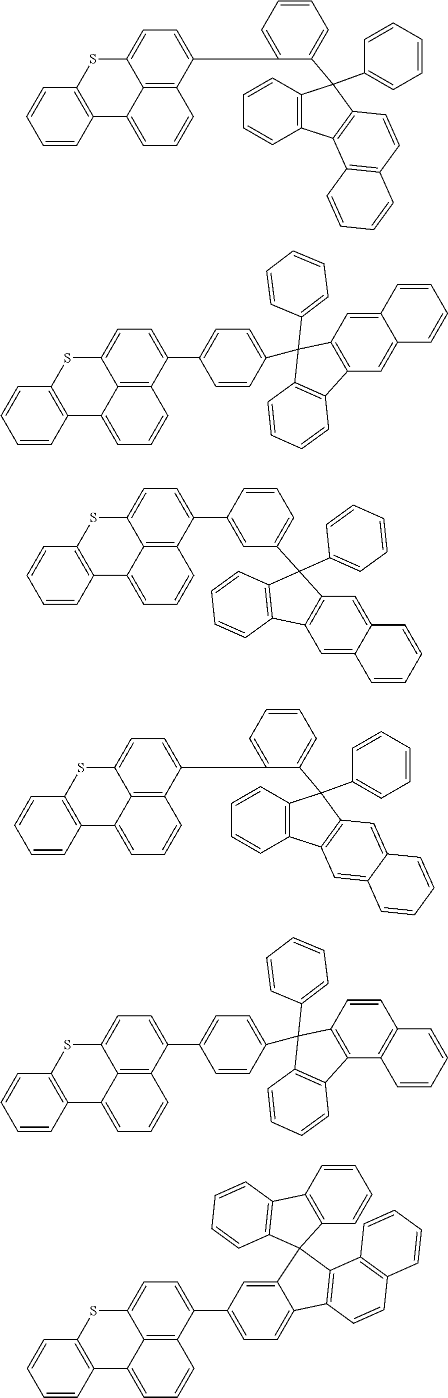

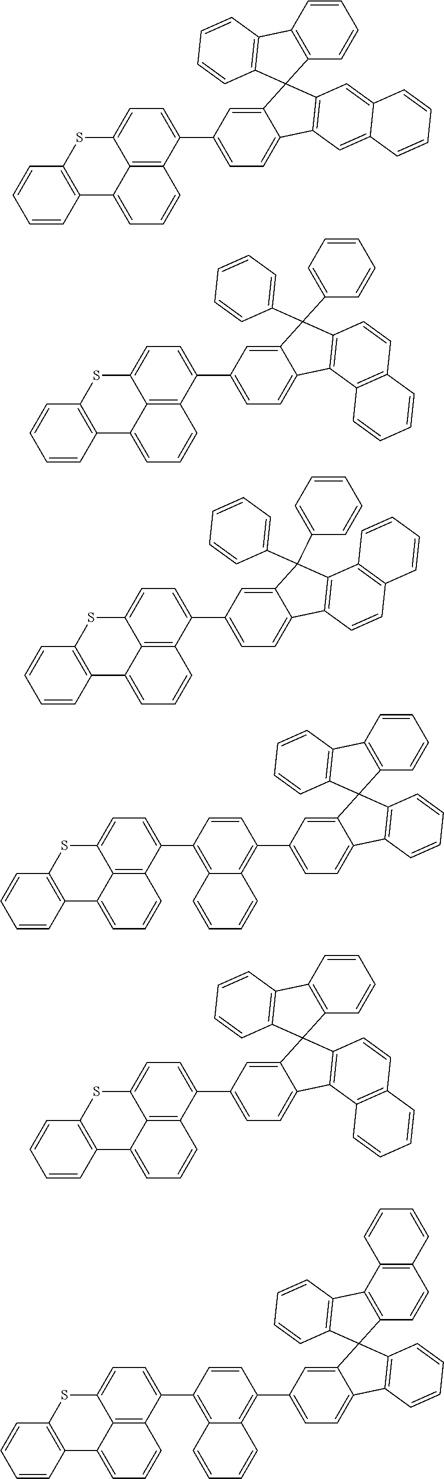

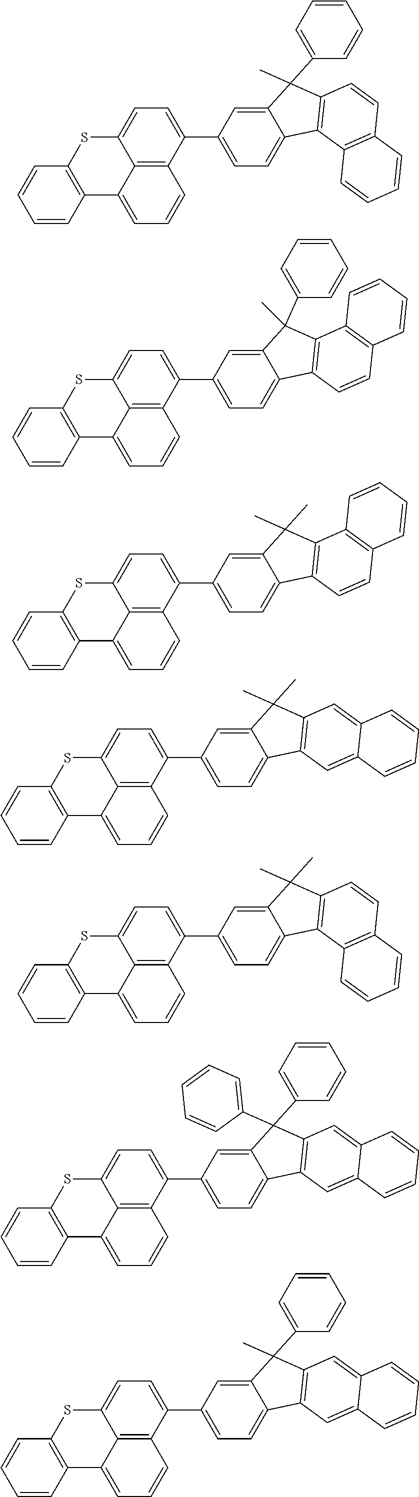

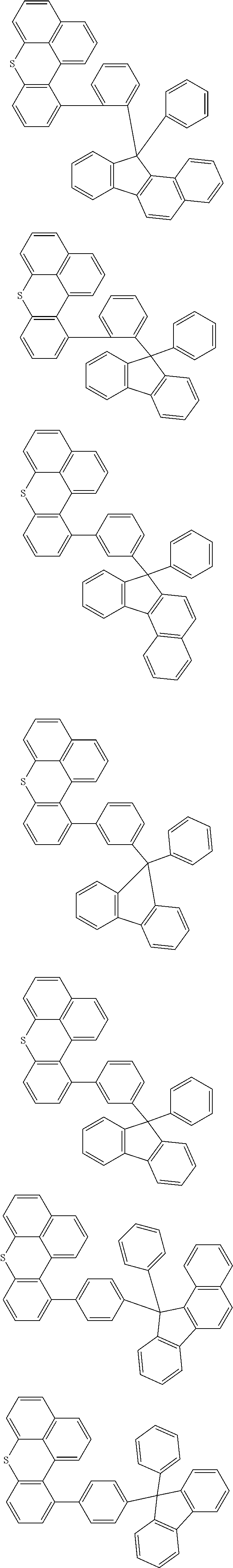

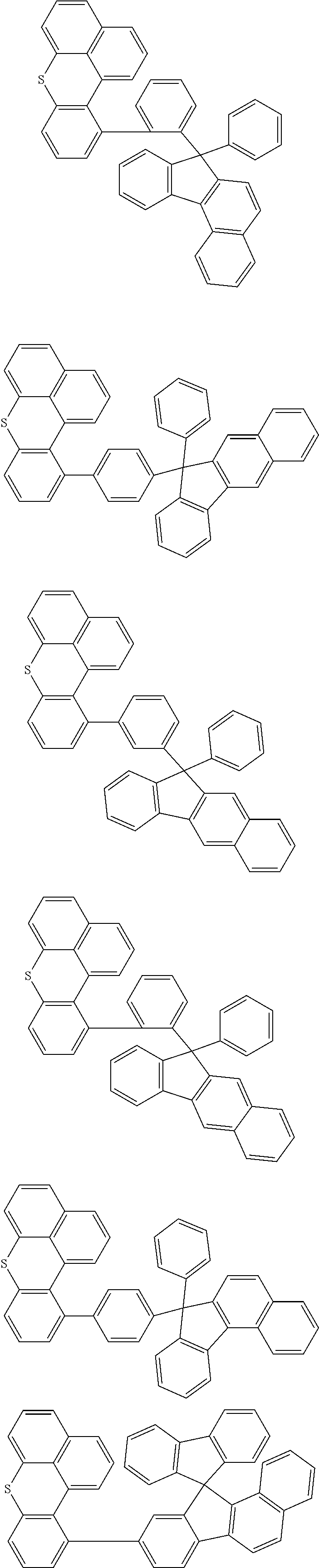

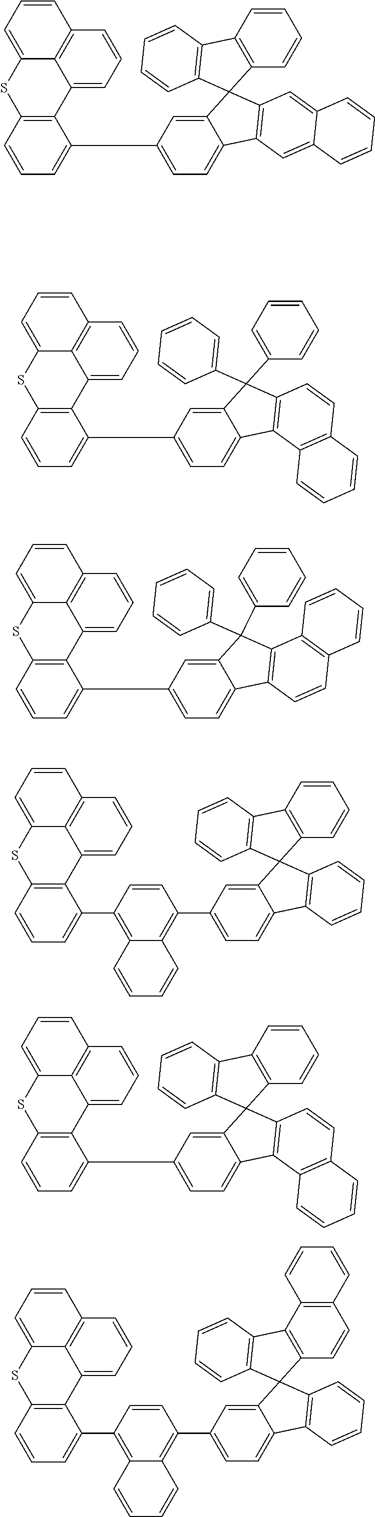

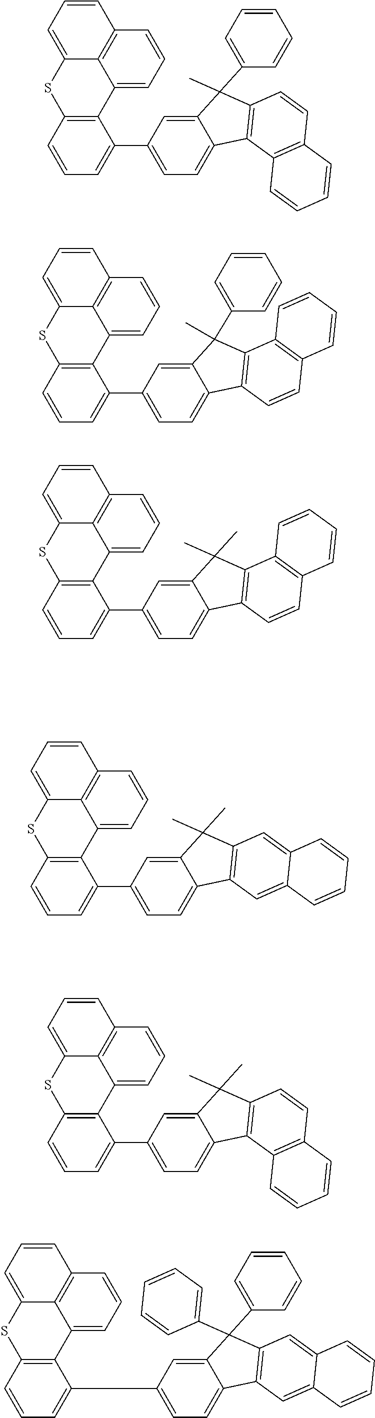

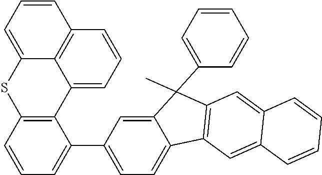 [SMILES: CC1(c2ccccc2)c2cc(-c3cccc4c3-c3cccc5cccc(c35)S4)ccc2-c2cc3ccccc3cc21]

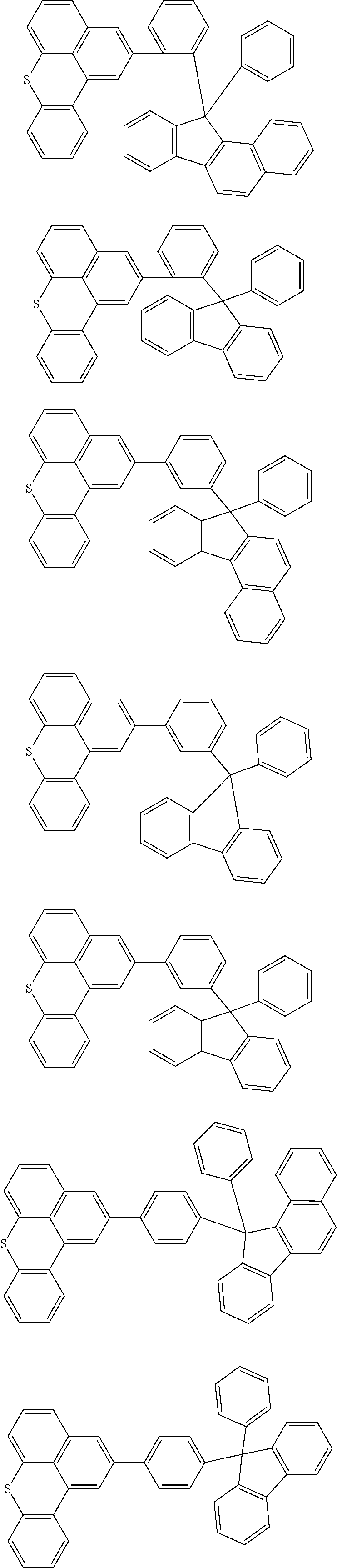

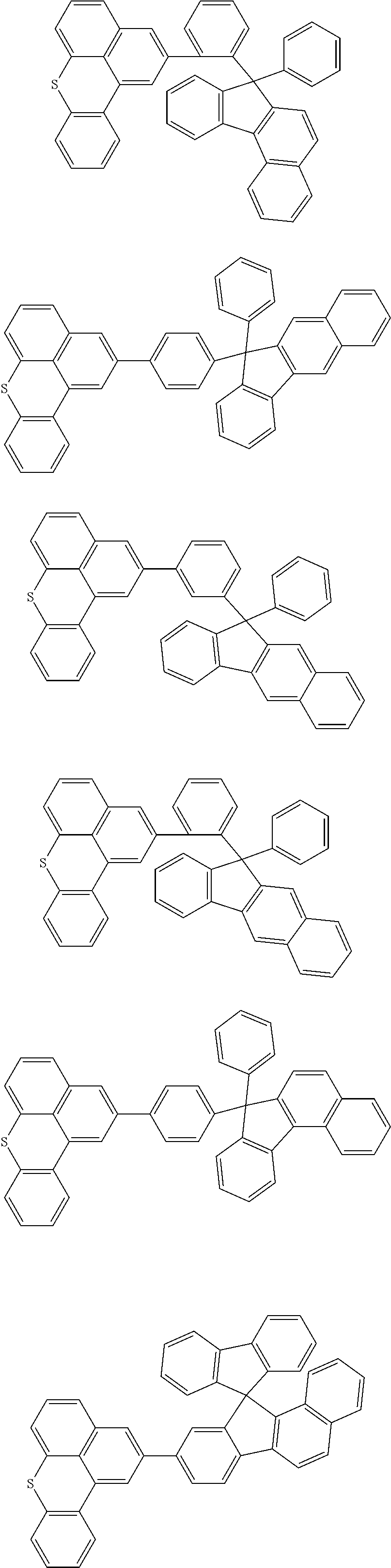

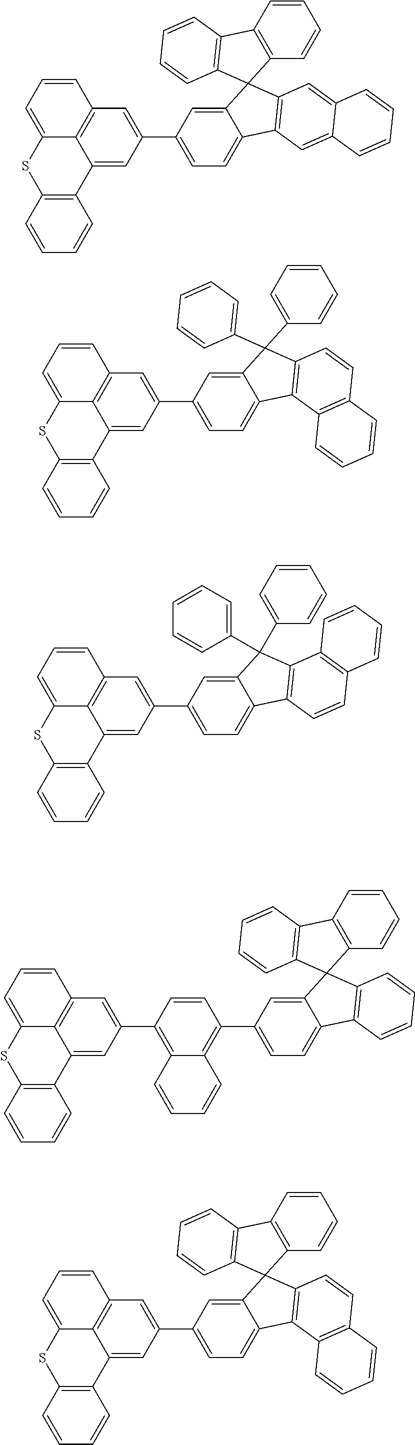 [SMILES: c1ccc2c(c1)Sc1cccc3cc(-c4ccc5c(c4)C4(c6ccccc6-c6ccccc64)c4cc6ccccc6cc4-5)cc-2c13 ; c1ccc(C2(c3ccccc3)c3cc(-c4cc5c6c(cccc6c4)Sc4ccccc4-5)ccc3-c3c2ccc2ccccc32)cc1 ; c1ccc(C2(c3ccccc3)c3cc(-c4cc5c6c(cccc6c4)Sc4ccccc4-5)ccc3-c3ccc4ccccc4c32)cc1 ; c1ccc2c(c1)Sc1cccc3cc(-c4ccc(-c5ccc6c(c5)C5(c7ccccc7-c7ccccc75)c5ccccc5-6)c5ccccc45)cc-2c13 ; c1ccc2c(c1)Sc1cccc3cc(-c4ccc5c(c4)C4(c6ccccc6-c6ccccc64)c4ccc6ccccc6c4-5)cc-2c13]

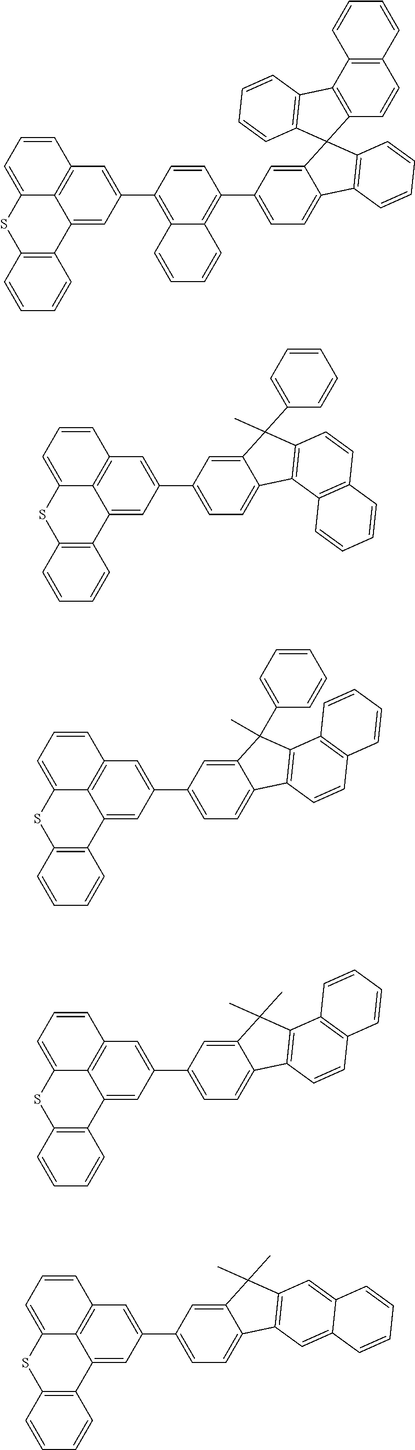 [SMILES: c1ccc2c(c1)Sc1cccc3cc(-c4ccc(-c5ccc6c(c5)C5(c7ccccc7-6)c6ccccc6-c6c5ccc5ccccc65)c5ccccc45)cc-2c13 ; CC1(c2ccccc2)c2cc(-c3cc4c5c(cccc5c3)Sc3ccccc3-4)ccc2-c2c1ccc1ccccc21 ; CC1(c2ccccc2)c2cc(-c3cc4c5c(cccc5c3)Sc3ccccc3-4)ccc2-c2ccc3ccccc3c21 ; CC1(C)c2cc(-c3cc4c5c(cccc5c3)Sc3ccccc3-4)ccc2-c2ccc3ccccc3c21 ; CC1(C)c2cc(-c3cc4c5c(cccc5c3)Sc3ccccc3-4)ccc2-c2cc3ccccc3cc21]

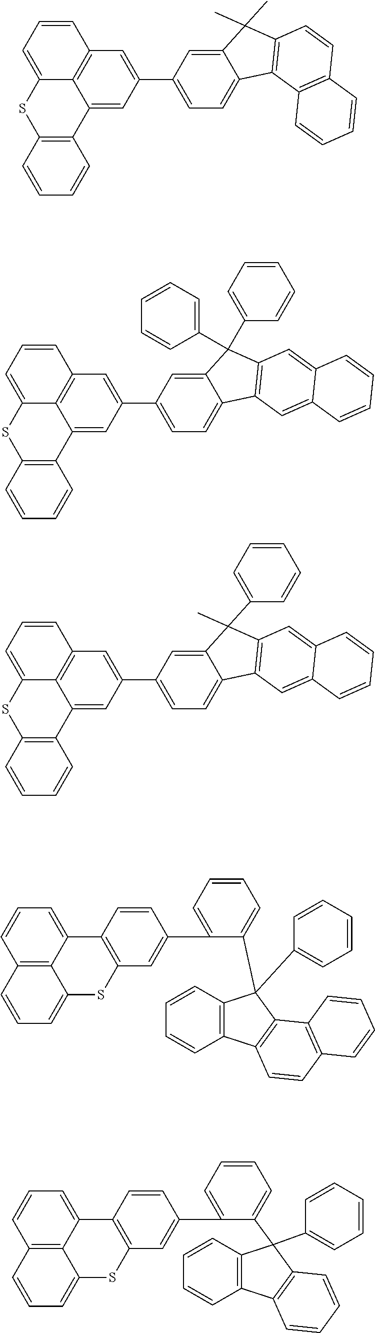 [SMILES: CC1(C)c2cc(-c3cc4c5c(cccc5c3)Sc3ccccc3-4)ccc2-c2c1ccc1ccccc21 ; c1ccc(C2(c3ccccc3)c3cc(-c4cc5c6c(cccc6c4)Sc4ccccc4-5)ccc3-c3cc4ccccc4cc32)cc1 ; CC1(c2ccccc2)c2cc(-c3cc4c5c(cccc5c3)Sc3ccccc3-4)ccc2-c2cc3ccccc3cc21 ; c1ccc(C2(c3ccccc3-c3ccc4c(c3)Sc3cccc5cccc-4c35)c3ccccc3-c3ccc4ccccc4c32)cc1 ; c1ccc(C2(c3ccccc3-c3ccc4c(c3)Sc3cccc5cccc-4c35)c3ccccc3-c3ccccc32)cc1]

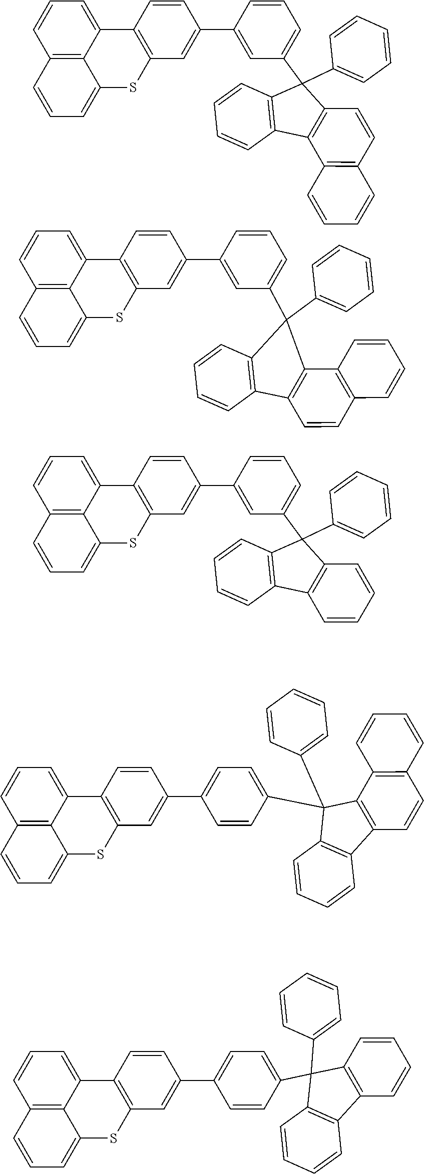

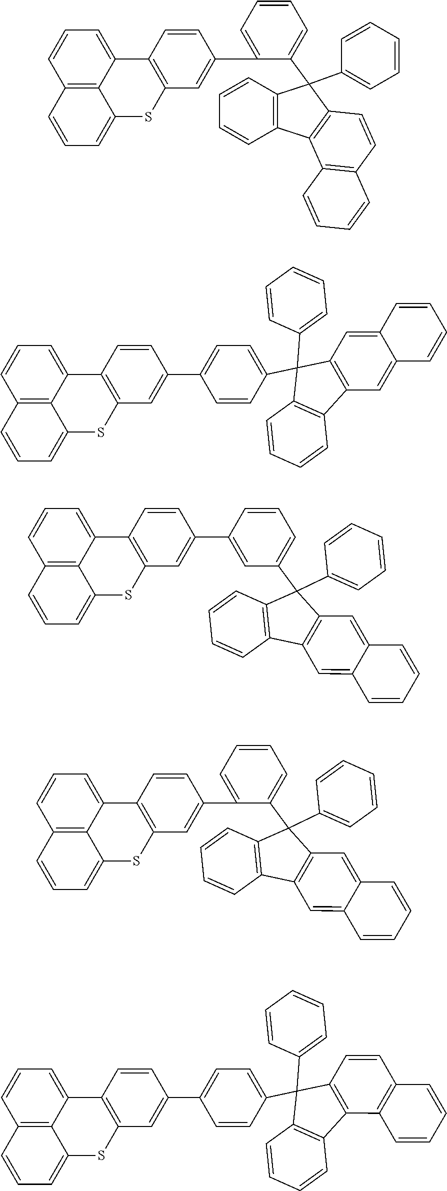

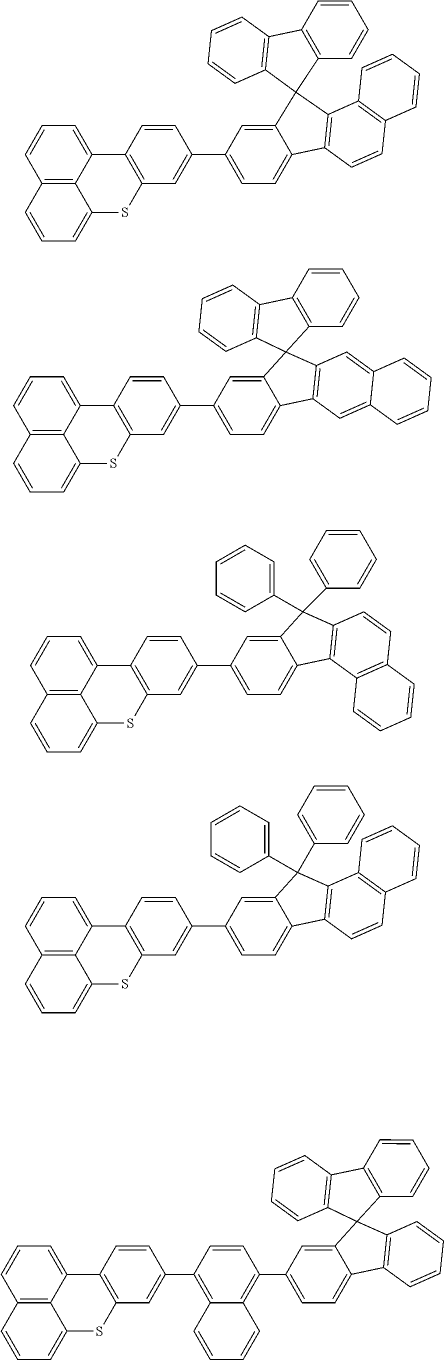 [SMILES: c1ccc2c(c1)-c1ccccc1C21c2cc(-c3ccc4c(c3)Sc3cccc5cccc-4c35)ccc2-c2ccc3ccccc3c21 ; c1ccc2c(c1)-c1ccccc1C21c2cc(-c3ccc4c(c3)Sc3cccc5cccc-4c35)ccc2-c2cc3ccccc3cc21 ; c1ccc(C2(c3ccccc3)c3cc(-c4ccc5c(c4)Sc4cccc6cccc-5c46)ccc3-c3c2ccc2ccccc32)cc1 ; c1ccc(C2(c3ccccc3)c3cc(-c4ccc5c(c4)Sc4cccc6cccc-5c46)ccc3-c3ccc4ccccc4c32)cc1 ; c1ccc2c(c1)-c1ccccc1C21c2ccccc2-c2ccc(-c3ccc(-c4ccc5c(c4)Sc4cccc6cccc-5c46)c4ccccc34)cc21]

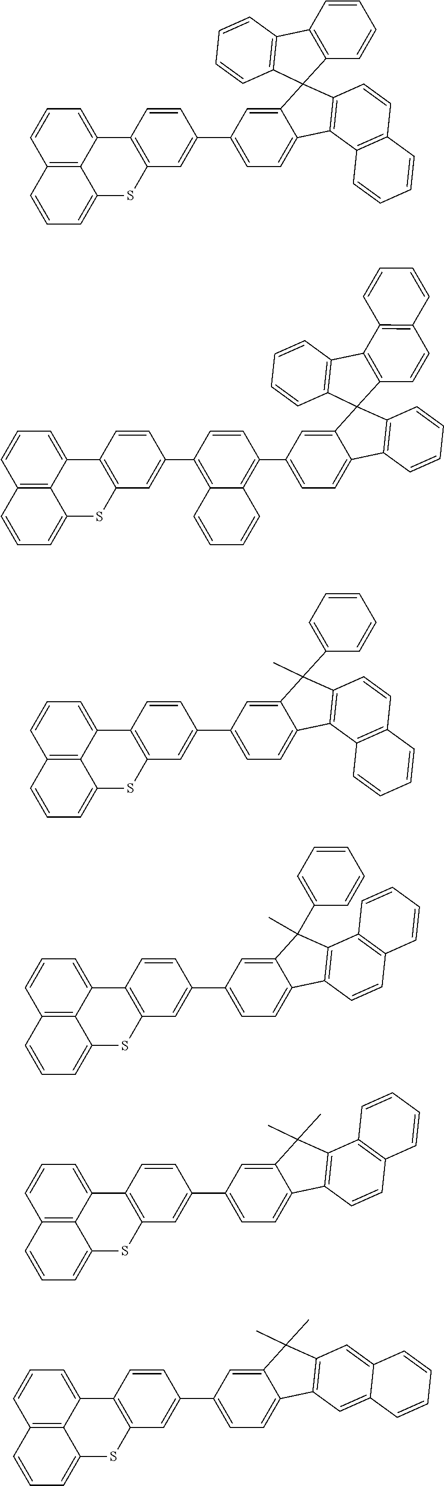 [SMILES: c1ccc2c(c1)-c1ccccc1C21c2cc(-c3ccc4c(c3)Sc3cccc5cccc-4c35)ccc2-c2c1ccc1ccccc21 ; c1ccc2c(c1)-c1ccc(-c3ccc(-c4ccc5c(c4)Sc4cccc6cccc-5c46)c4ccccc34)cc1C21c2ccccc2-c2c1ccc1ccccc21 ; CC1(c2ccccc2)c2cc(-c3ccc4c(c3)Sc3cccc5cccc-4c35)ccc2-c2c1ccc1ccccc21 ; CC1(c2ccccc2)c2cc(-c3ccc4c(c3)Sc3cccc5cccc-4c35)ccc2-c2ccc3ccccc3c21 ; CC1(C)c2cc(-c3ccc4c(c3)Sc3cccc5cccc-4c35)ccc2-c2ccc3ccccc3c21 ; CC1(C)c2cc(-c3ccc4c(c3)Sc3cccc5cccc-4c35)ccc2-c2cc3ccccc3cc21]

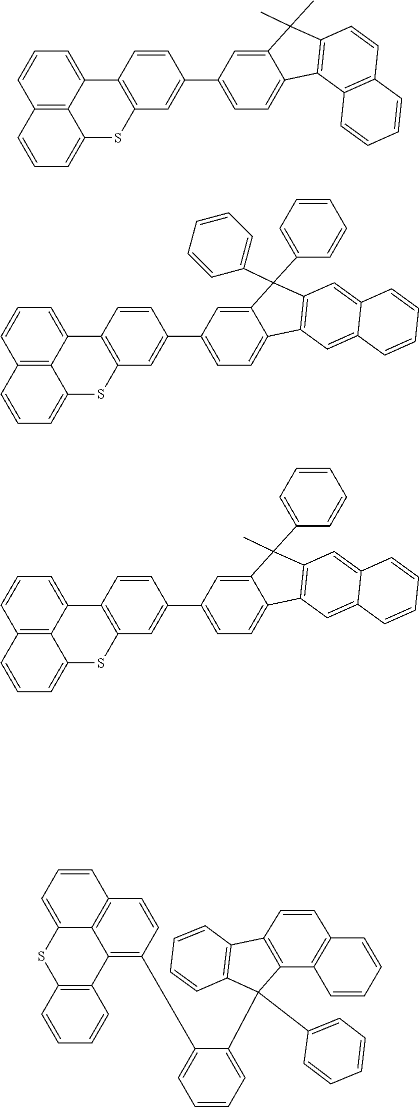 [SMILES: CC1(C)c2cc(-c3ccc4c(c3)Sc3cccc5cccc-4c35)ccc2-c2c1ccc1ccccc21 ; c1ccc(C2(c3ccccc3)c3cc(-c4ccc5c(c4)Sc4cccc6cccc-5c46)ccc3-c3cc4ccccc4cc32)cc1 ; CC1(c2ccccc2)c2cc(-c3ccc4c(c3)Sc3cccc5cccc-4c35)ccc2-c2cc3ccccc3cc21 ; c1ccc(C2(c3ccccc3-c3ccc4cccc5c4c3-c3ccccc3S5)c3ccccc3-c3ccc4ccccc4c32)cc1]

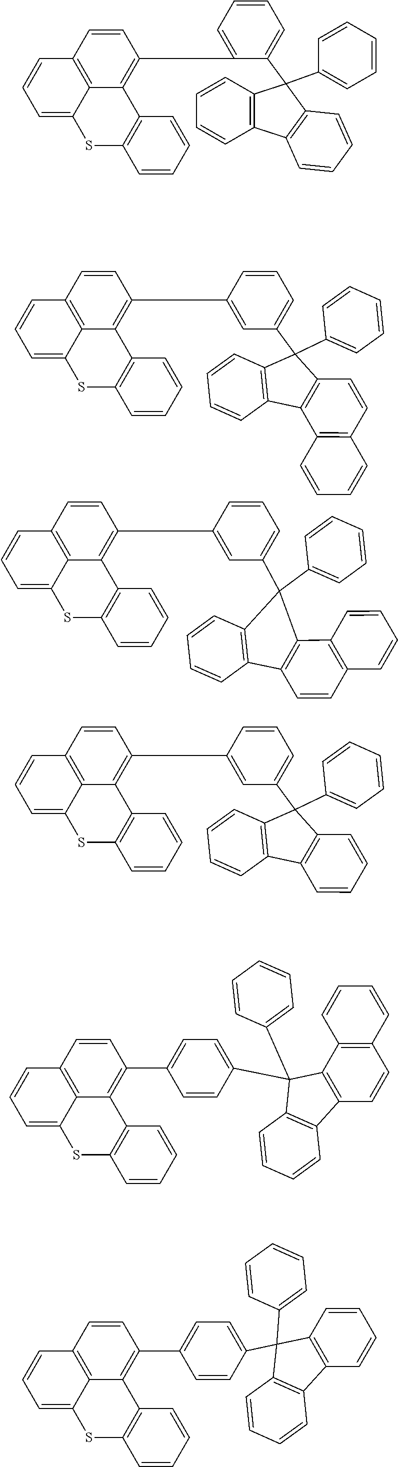

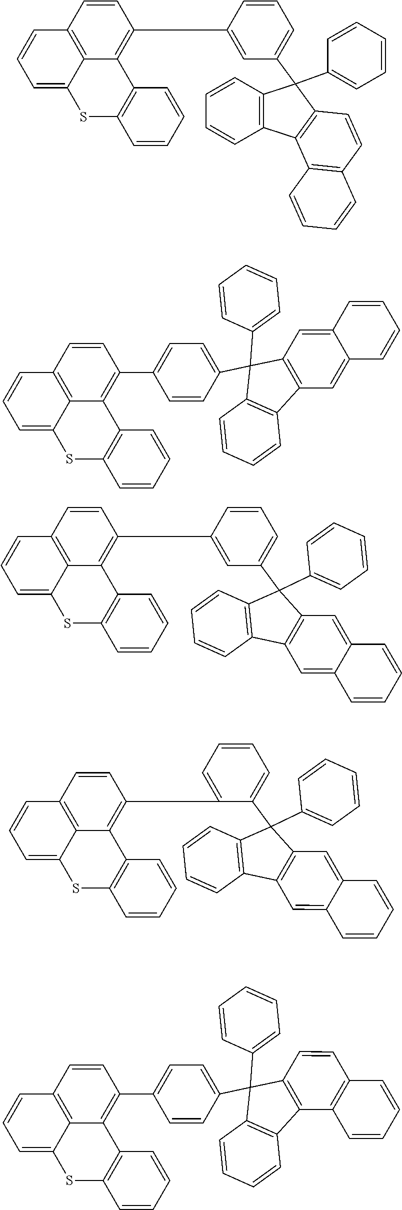

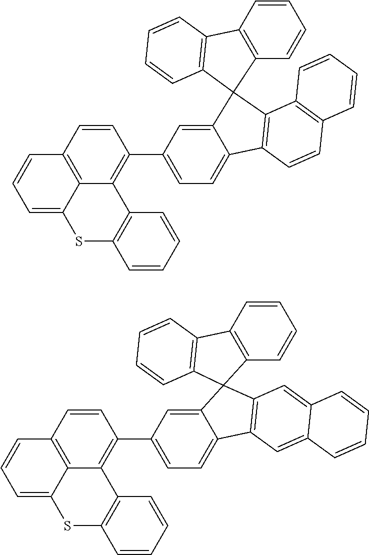 [SMILES: c1ccc2c(c1)Sc1cccc3ccc(-c4ccc5c(c4)C4(c6ccccc6-c6ccccc64)c4c-5ccc5ccccc45)c-2c13 ; c1ccc2c(c1)Sc1cccc3ccc(-c4ccc5c(c4)C4(c6ccccc6-c6ccccc64)c4cc6ccccc6cc4-5)c-2c13]

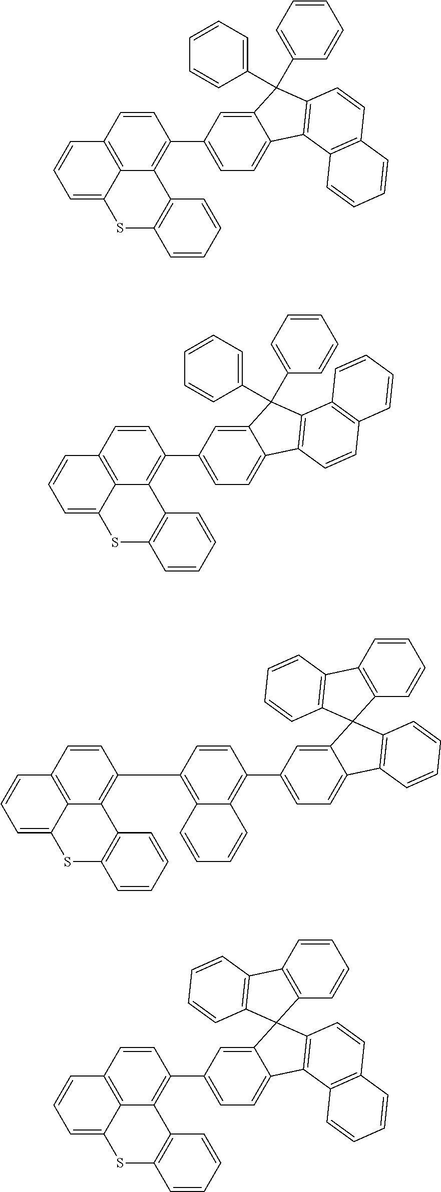 [SMILES: c1ccc(C2(c3ccccc3)c3cc(-c4ccc5cccc6c5c4-c4ccccc4S6)ccc3-c3c2ccc2ccccc32)cc1 ; c1ccc(C2(c3ccccc3)c3cc(-c4ccc5cccc6c5c4-c4ccccc4S6)ccc3-c3ccc4ccccc4c32)cc1 ; c1ccc2c(c1)Sc1cccc3ccc(-c4ccc(-c5ccc6c(c5)C5(c7ccccc7-c7ccccc75)c5ccccc5-6)c5ccccc45)c-2c13 ; c1ccc2c(c1)Sc1cccc3ccc(-c4ccc5c(c4)C4(c6ccccc6-c6ccccc64)c4ccc6ccccc6c4-5)c-2c13]

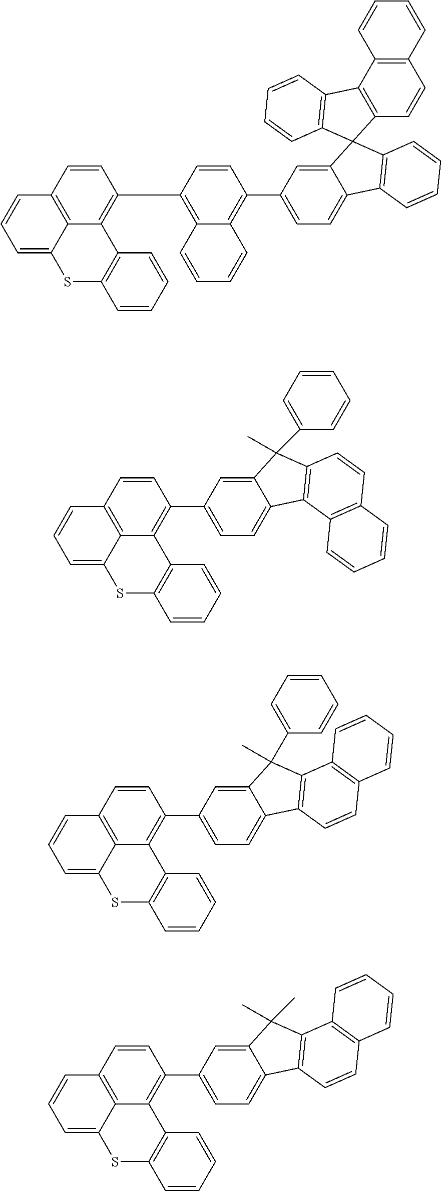 [SMILES: c1ccc2c(c1)Sc1cccc3ccc(-c4ccc(-c5ccc6c(c5)C5(c7ccccc7-6)c6ccccc6-c6c5ccc5ccccc65)c5ccccc45)c-2c13 ; CC1(c2ccccc2)c2cc(-c3ccc4cccc5c4c3-c3ccccc3S5)ccc2-c2c1ccc1ccccc21 ; CC1(c2ccccc2)c2cc(-c3ccc4cccc5c4c3-c3ccccc3S5)ccc2-c2ccc3ccccc3c21 ; CC1(C)c2cc(-c3ccc4cccc5c4c3-c3ccccc3S5)ccc2-c2ccc3ccccc3c21]

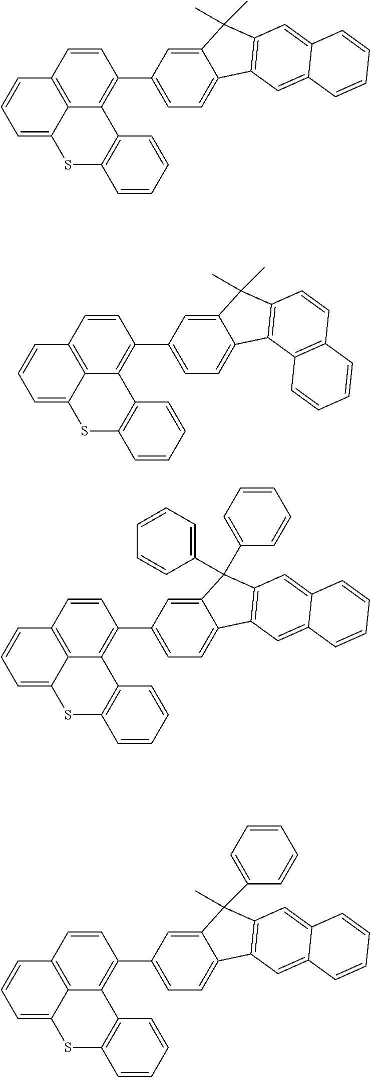 [SMILES: CC1(C)c2cc(-c3ccc4cccc5c4c3-c3ccccc3S5)ccc2-c2cc3ccccc3cc21 ; CC1(C)c2cc(-c3ccc4cccc5c4c3-c3ccccc3S5)ccc2-c2c1ccc1ccccc21 ; c1ccc(C2(c3ccccc3)c3cc(-c4ccc5cccc6c5c4-c4ccccc4S6)ccc3-c3cc4ccccc4cc32)cc1 ; CC1(c2ccccc2)c2cc(-c3ccc4cccc5c4c3-c3ccccc3S5)ccc2-c2cc3ccccc3cc21]

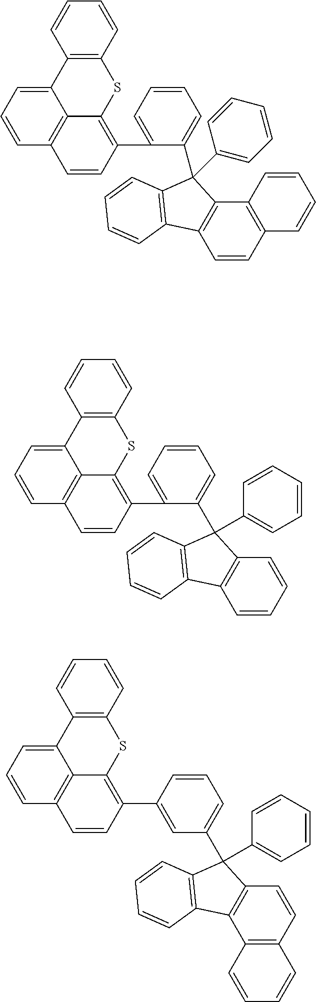 [SMILES: c1ccc(C2(c3ccccc3-c3ccc4cccc5c4c3Sc3ccccc3-5)c3ccccc3-c3ccc4ccccc4c32)cc1 ; c1ccc(C2(c3ccccc3-c3ccc4cccc5c4c3Sc3ccccc3-5)c3ccccc3-c3ccccc32)cc1 ; c1ccc(C2(c3cccc(-c4ccc5cccc6c5c4Sc4ccccc4-6)c3)c3ccccc3-c3c2ccc2ccccc32)cc1]

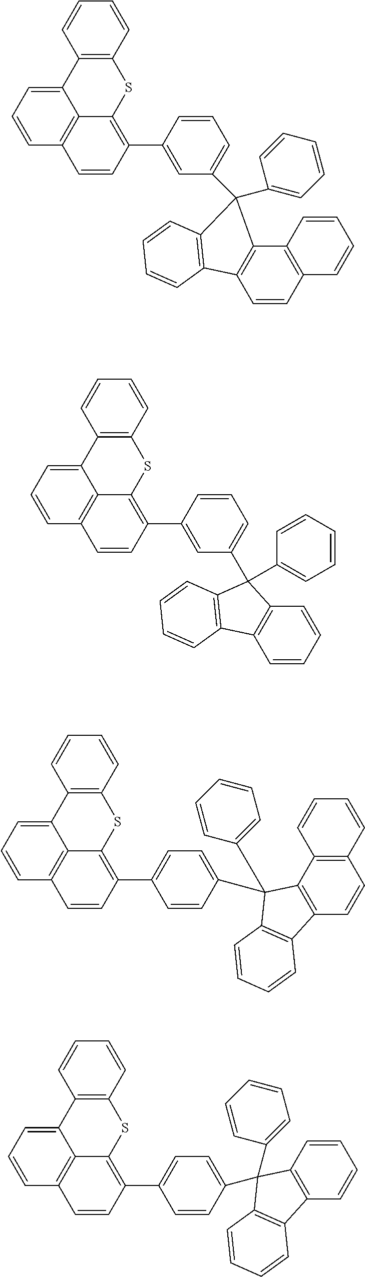 [SMILES: c1ccc(C2(c3cccc(-c4ccc5cccc6c5c4Sc4ccccc4-6)c3)c3ccccc3-c3ccc4ccccc4c32)cc1 ; c1ccc(C2(c3cccc(-c4ccc5cccc6c5c4Sc4ccccc4-6)c3)c3ccccc3-c3ccccc32)cc1 ; c1ccc(C2(c3ccc(-c4ccc5cccc6c5c4Sc4ccccc4-6)cc3)c3ccccc3-c3ccc4ccccc4c32)cc1 ; c1ccc(C2(c3ccc(-c4ccc5cccc6c5c4Sc4ccccc4-6)cc3)c3ccccc3-c3ccccc32)cc1]

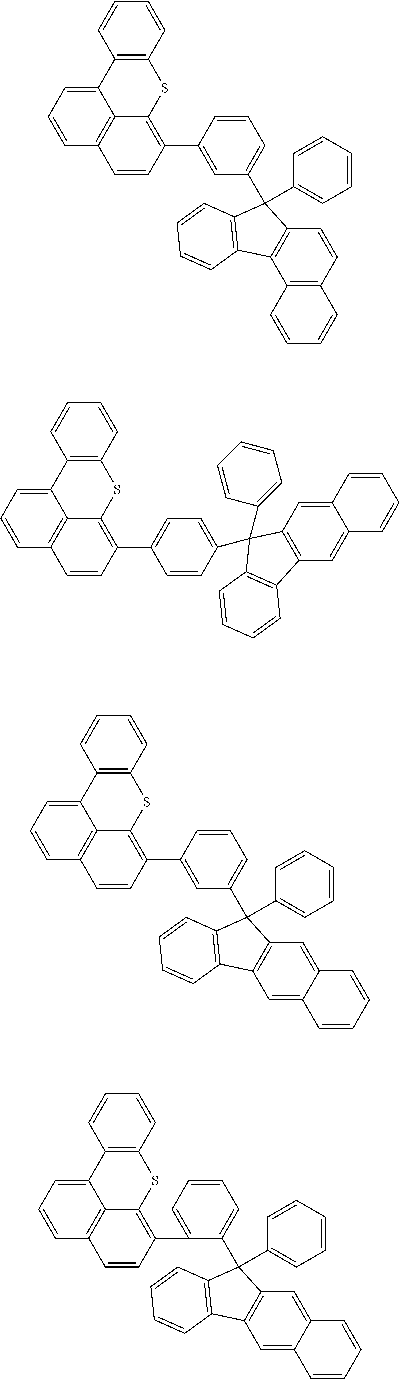 [SMILES: c1ccc(C2(c3cccc(-c4ccc5cccc6c5c4Sc4ccccc4-6)c3)c3ccccc3-c3c2ccc2ccccc32)cc1 ; c1ccc(C2(c3ccc(-c4ccc5cccc6c5c4Sc4ccccc4-6)cc3)c3ccccc3-c3cc4ccccc4cc32)cc1 ; c1ccc(C2(c3cccc(-c4ccc5cccc6c5c4Sc4ccccc4-6)c3)c3ccccc3-c3cc4ccccc4cc32)cc1 ; c1ccc(C2(c3ccccc3-c3ccc4cccc5c4c3Sc3ccccc3-5)c3ccccc3-c3cc4ccccc4cc32)cc1]

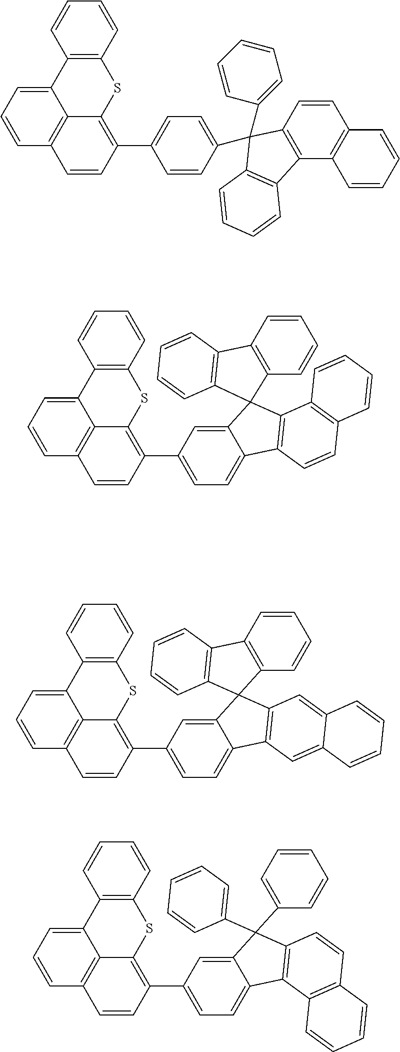 [SMILES: c1ccc(C2(c3ccc(-c4ccc5cccc6c5c4Sc4ccccc4-6)cc3)c3ccccc3-c3c2ccc2ccccc32)cc1 ; c1ccc2c(c1)Sc1c(-c3ccc4c(c3)C3(c5ccccc5-c5ccccc53)c3c-4ccc4ccccc34)ccc3cccc-2c13 ; c1ccc2c(c1)Sc1c(-c3ccc4c(c3)C3(c5ccccc5-c5ccccc53)c3cc5ccccc5cc3-4)ccc3cccc-2c13 ; c1ccc(C2(c3ccccc3)c3cc(-c4ccc5cccc6c5c4Sc4ccccc4-6)ccc3-c3c2ccc2ccccc32)cc1]

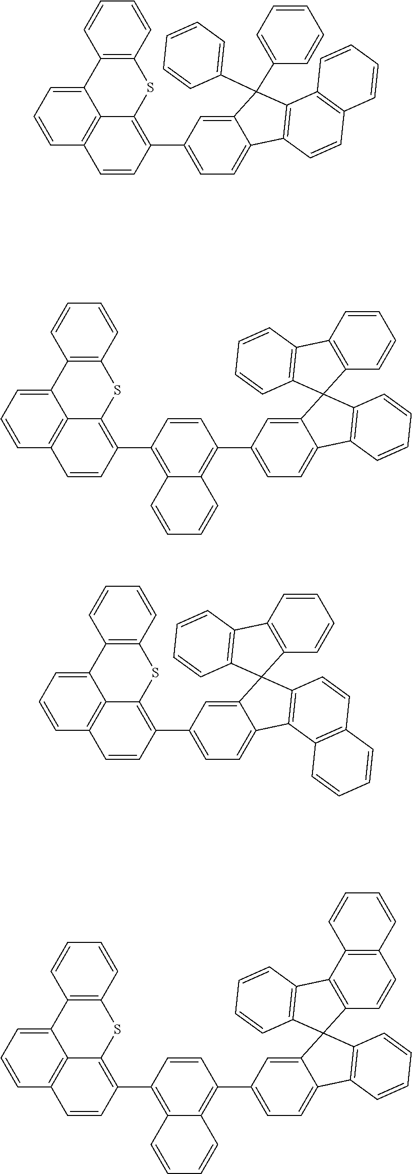 [SMILES: c1ccc(C2(c3ccccc3)c3cc(-c4ccc5cccc6c5c4Sc4ccccc4-6)ccc3-c3ccc4ccccc4c32)cc1 ; c1ccc2c(c1)Sc1c(-c3ccc(-c4ccc5c(c4)C4(c6ccccc6-c6ccccc64)c4ccccc4-5)c4ccccc34)ccc3cccc-2c13 ; c1ccc2c(c1)Sc1c(-c3ccc4c(c3)C3(c5ccccc5-c5ccccc53)c3ccc5ccccc5c3-4)ccc3cccc-2c13 ; c1ccc2c(c1)Sc1c(-c3ccc(-c4ccc5c(c4)C4(c6ccccc6-5)c5ccccc5-c5c4ccc4ccccc54)c4ccccc34)ccc3cccc-2c13]

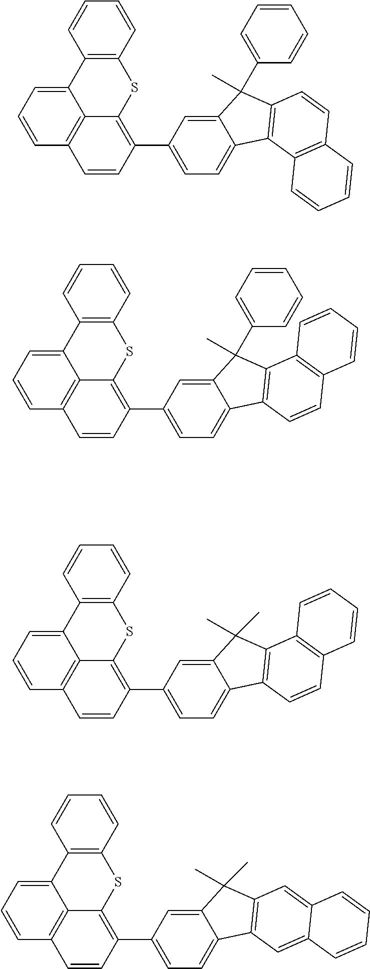 [SMILES: CC1(c2ccccc2)c2cc(-c3ccc4cccc5c4c3Sc3ccccc3-5)ccc2-c2c1ccc1ccccc21 ; CC1(c2ccccc2)c2cc(-c3ccc4cccc5c4c3Sc3ccccc3-5)ccc2-c2ccc3ccccc3c21 ; CC1(C)c2cc(-c3ccc4cccc5c4c3Sc3ccccc3-5)ccc2-c2ccc3ccccc3c21 ; CC1(C)c2cc(-c3ccc4cccc5c4c3Sc3ccccc3-5)ccc2-c2cc3ccccc3cc21]

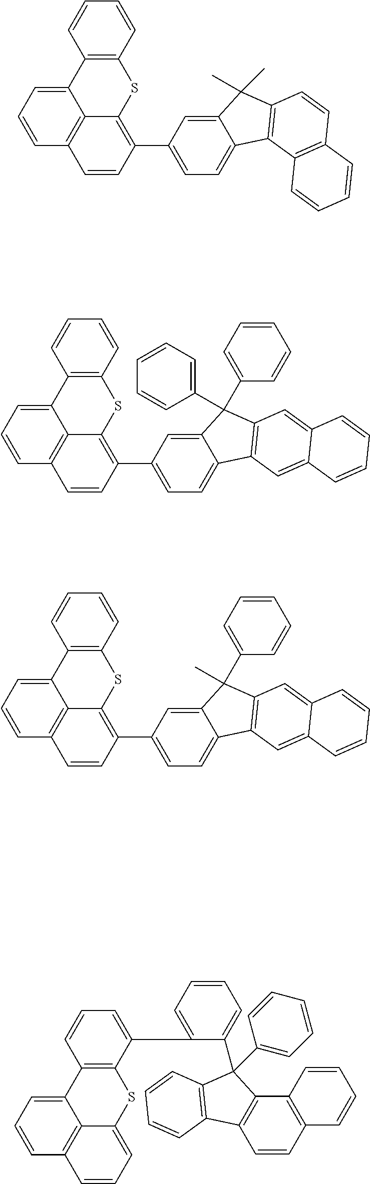 [SMILES: CC1(C)c2cc(-c3ccc4cccc5c4c3Sc3ccccc3-5)ccc2-c2c1ccc1ccccc21 ; c1ccc(C2(c3ccccc3)c3cc(-c4ccc5cccc6c5c4Sc4ccccc4-6)ccc3-c3cc4ccccc4cc32)cc1 ; CC1(c2ccccc2)c2cc(-c3ccc4cccc5c4c3Sc3ccccc3-5)ccc2-c2cc3ccccc3cc21 ; c1ccc(C2(c3ccccc3-c3cccc4c3Sc3cccc5cccc-4c35)c3ccccc3-c3ccc4ccccc4c32)cc1]

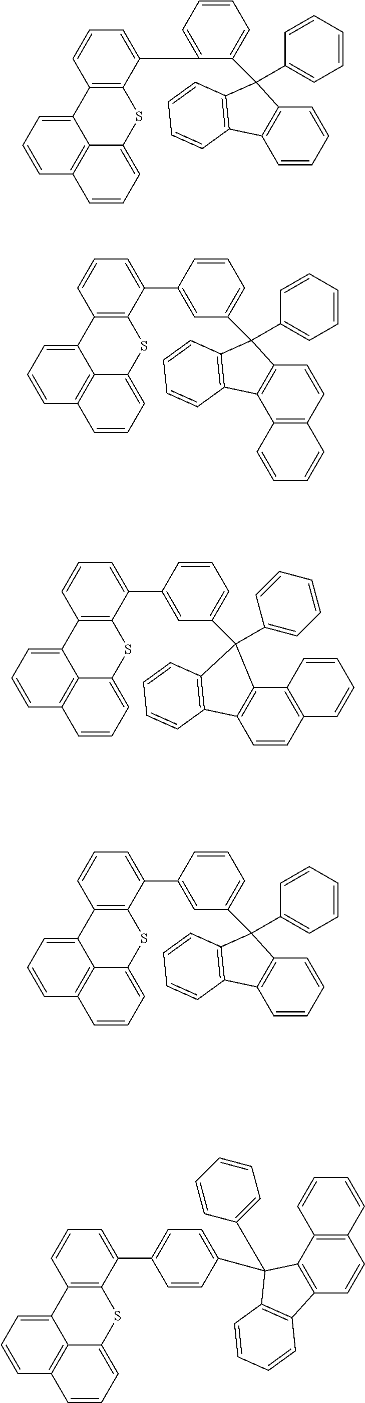 [SMILES: c1ccc(C2(c3ccccc3-c3cccc4c3Sc3cccc5cccc-4c35)c3ccccc3-c3ccccc32)cc1 ; c1ccc(C2(c3cccc(-c4cccc5c4Sc4cccc6cccc-5c46)c3)c3ccccc3-c3c2ccc2ccccc32)cc1 ; c1ccc(C2(c3cccc(-c4cccc5c4Sc4cccc6cccc-5c46)c3)c3ccccc3-c3ccc4ccccc4c32)cc1 ; c1ccc(C2(c3cccc(-c4cccc5c4Sc4cccc6cccc-5c46)c3)c3ccccc3-c3ccccc32)cc1 ; c1ccc(C2(c3ccc(-c4cccc5c4Sc4cccc6cccc-5c46)cc3)c3ccccc3-c3ccc4ccccc4c32)cc1]

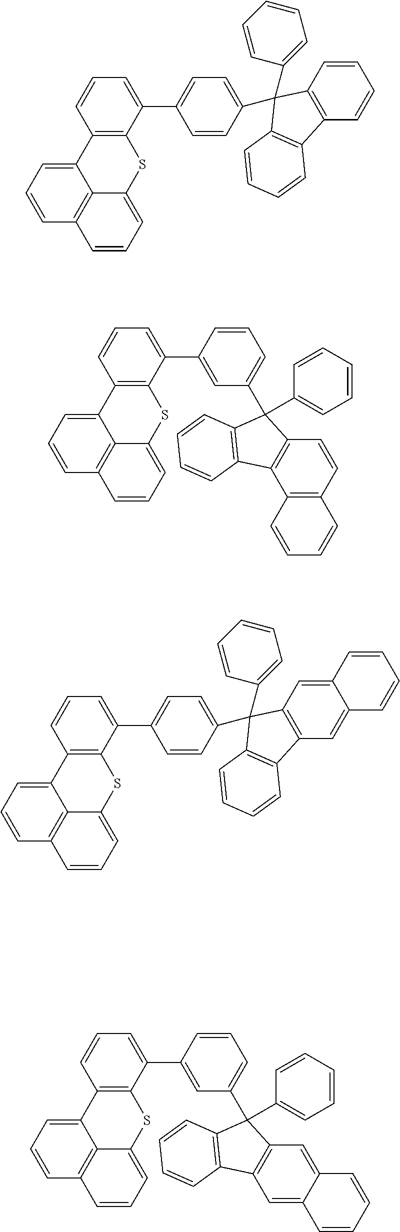 [SMILES: c1ccc(C2(c3ccc(-c4cccc5c4Sc4cccc6cccc-5c46)cc3)c3ccccc3-c3ccccc32)cc1 ; c1ccc(C2(c3cccc(-c4cccc5c4Sc4cccc6cccc-5c46)c3)c3ccccc3-c3c2ccc2ccccc32)cc1 ; c1ccc(C2(c3ccc(-c4cccc5c4Sc4cccc6cccc-5c46)cc3)c3ccccc3-c3cc4ccccc4cc32)cc1 ; c1ccc(C2(c3cccc(-c4cccc5c4Sc4cccc6cccc-5c46)c3)c3ccccc3-c3cc4ccccc4cc32)cc1]

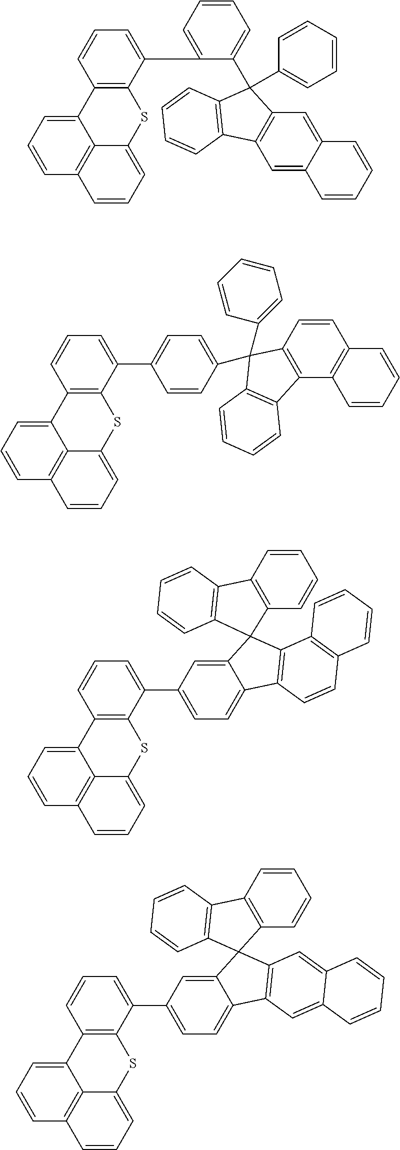 [SMILES: c1ccc(C2(c3ccccc3-c3cccc4c3Sc3cccc5cccc-4c35)c3ccccc3-c3cc4ccccc4cc32)cc1 ; c1ccc(C2(c3ccc(-c4cccc5c4Sc4cccc6cccc-5c46)cc3)c3ccccc3-c3c2ccc2ccccc32)cc1 ; c1ccc2c(c1)-c1ccccc1C21c2cc(-c3cccc4c3Sc3cccc5cccc-4c35)ccc2-c2ccc3ccccc3c21 ; c1ccc2c(c1)-c1ccccc1C21c2cc(-c3cccc4c3Sc3cccc5cccc-4c35)ccc2-c2cc3ccccc3cc21]

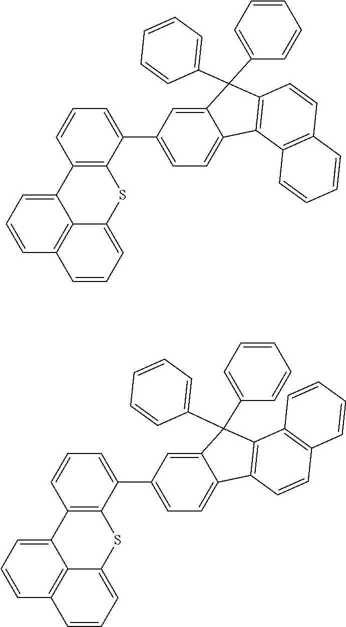 [SMILES: c1ccc(C2(c3ccccc3)c3cc(-c4cccc5c4Sc4cccc6cccc-5c46)ccc3-c3c2ccc2ccccc32)cc1 ; c1ccc(C2(c3ccccc3)c3cc(-c4cccc5c4Sc4cccc6cccc-5c46)ccc3-c3ccc4ccccc4c32)cc1]

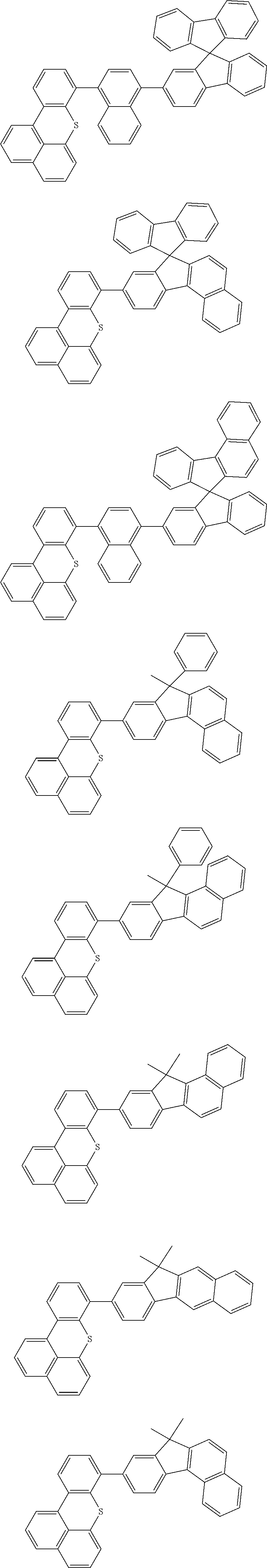

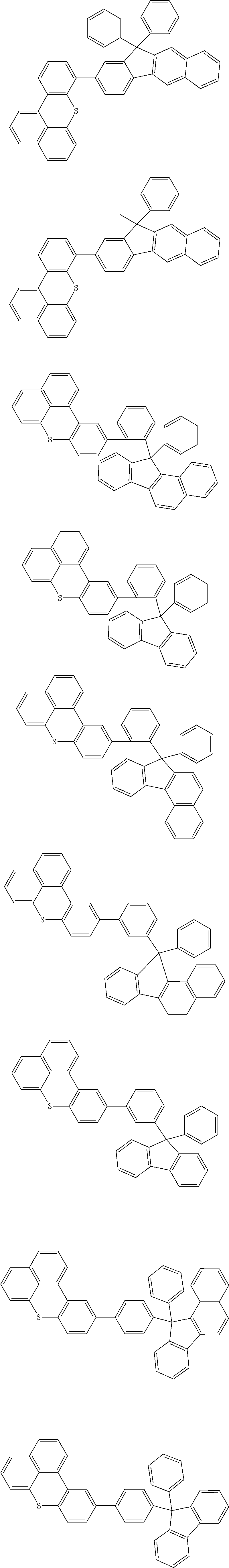

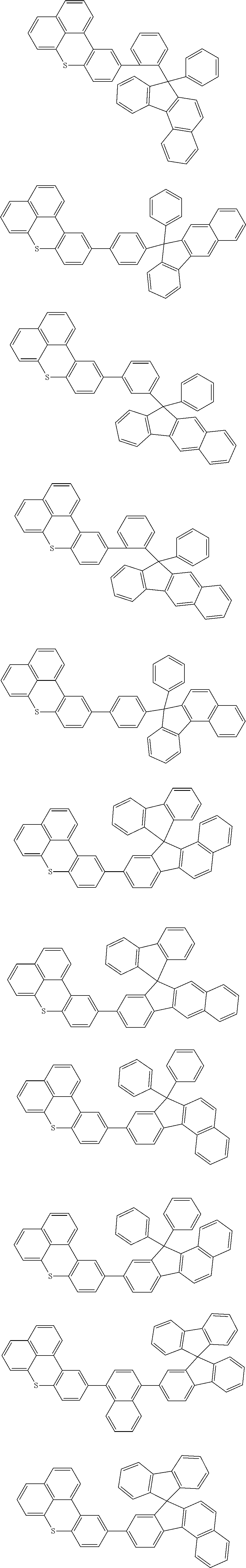

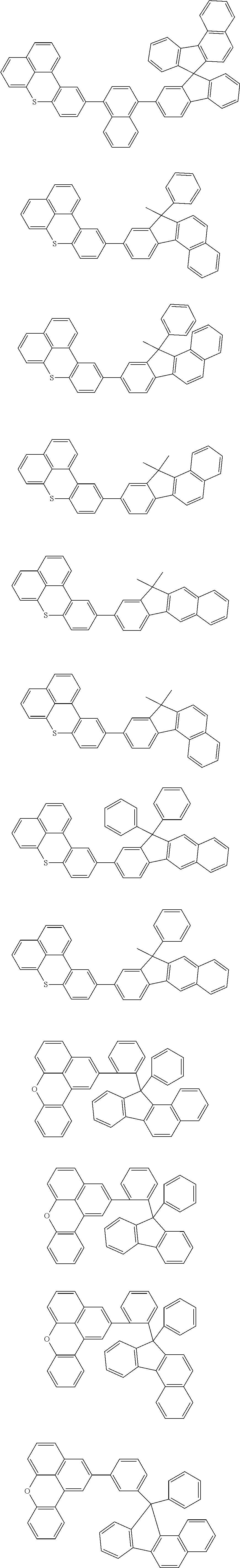

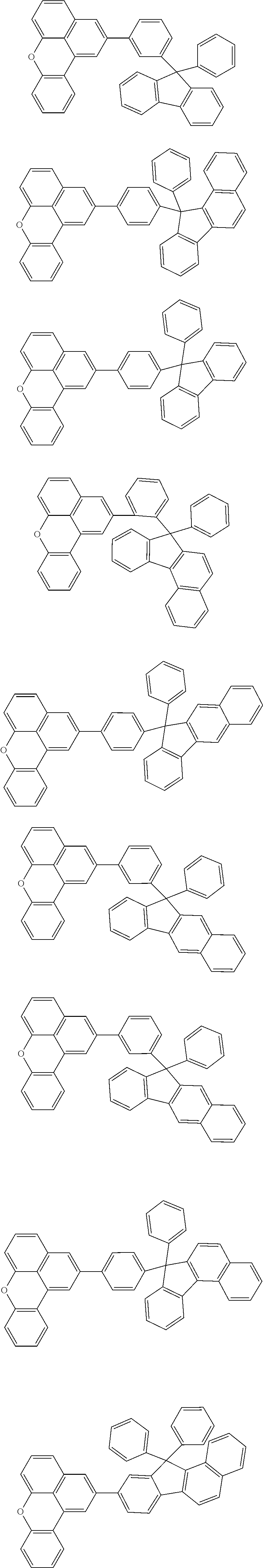

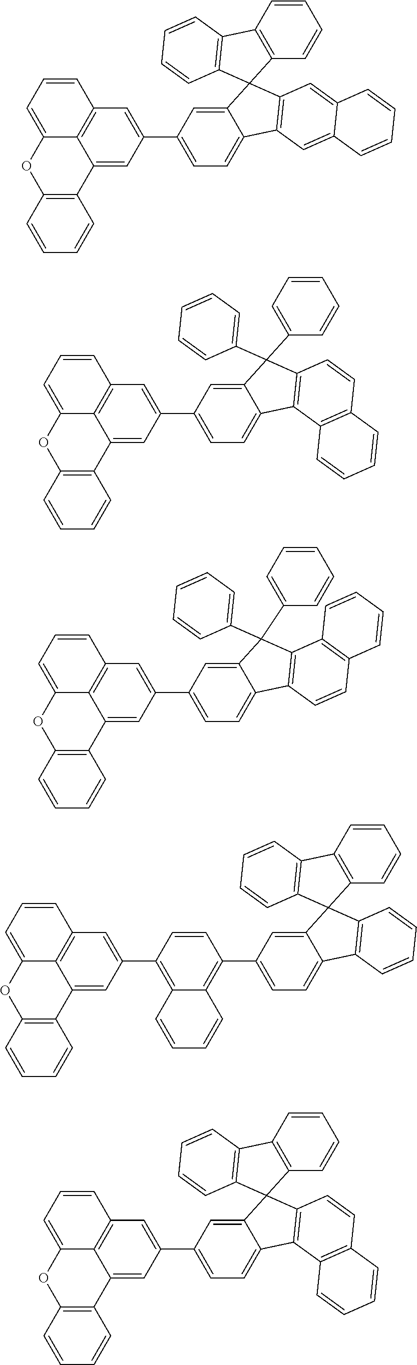 [SMILES: c1ccc2c(c1)Oc1cccc3cc(-c4ccc5c(c4)C4(c6ccccc6-c6ccccc64)c4cc6ccccc6cc4-5)cc-2c13 ; c1ccc(C2(c3ccccc3)c3cc(-c4cc5c6c(cccc6c4)Oc4ccccc4-5)ccc3-c3c2ccc2ccccc32)cc1 ; c1ccc(C2(c3ccccc3)c3cc(-c4cc5c6c(cccc6c4)Oc4ccccc4-5)ccc3-c3ccc4ccccc4c32)cc1 ; c1ccc2c(c1)Oc1cccc3cc(-c4ccc(-c5ccc6c(c5)C5(c7ccccc7-c7ccccc75)c5ccccc5-6)c5ccccc45)cc-2c13 ; c1ccc2c(c1)Oc1cccc3cc(-c4ccc5c(c4)C4(c6ccccc6-c6ccccc64)c4ccc6ccccc6c4-5)cc-2c13]

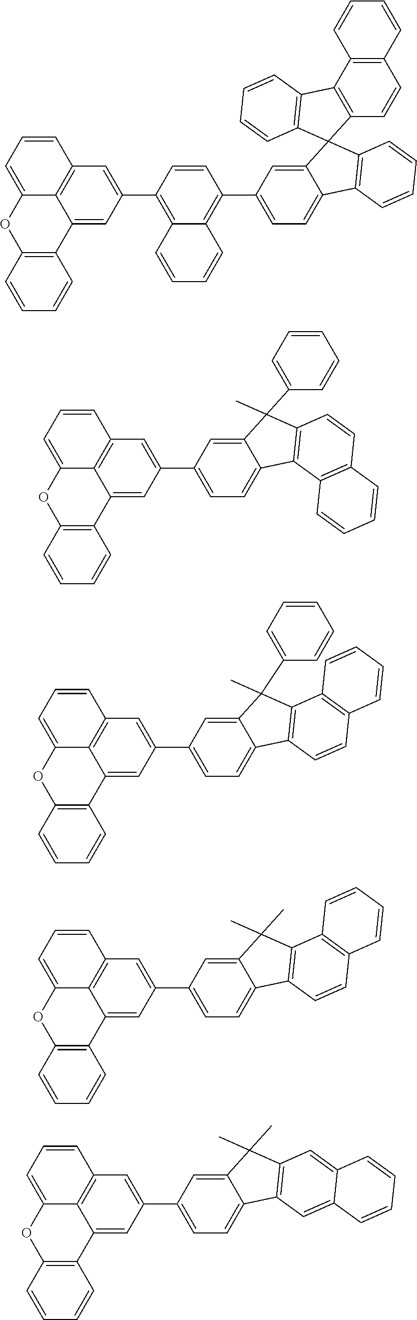

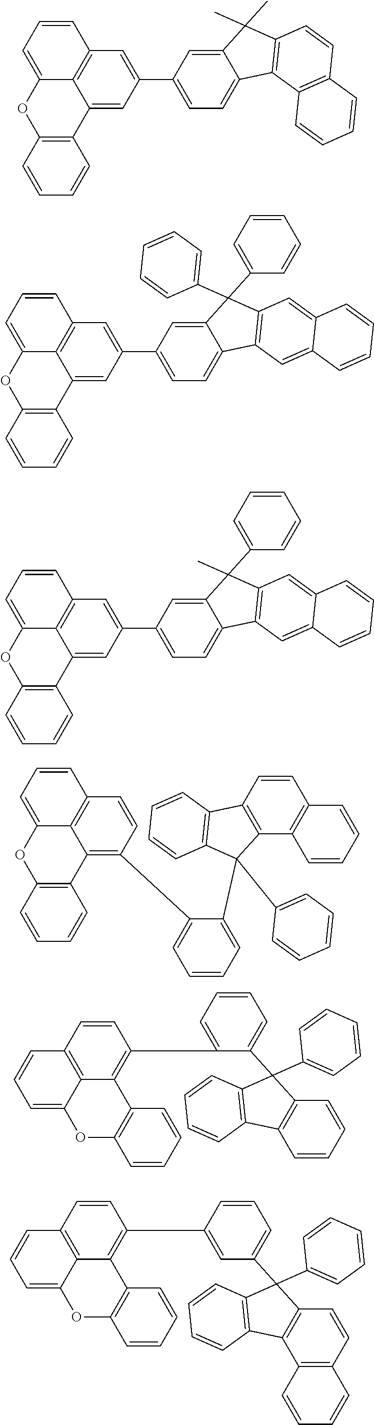

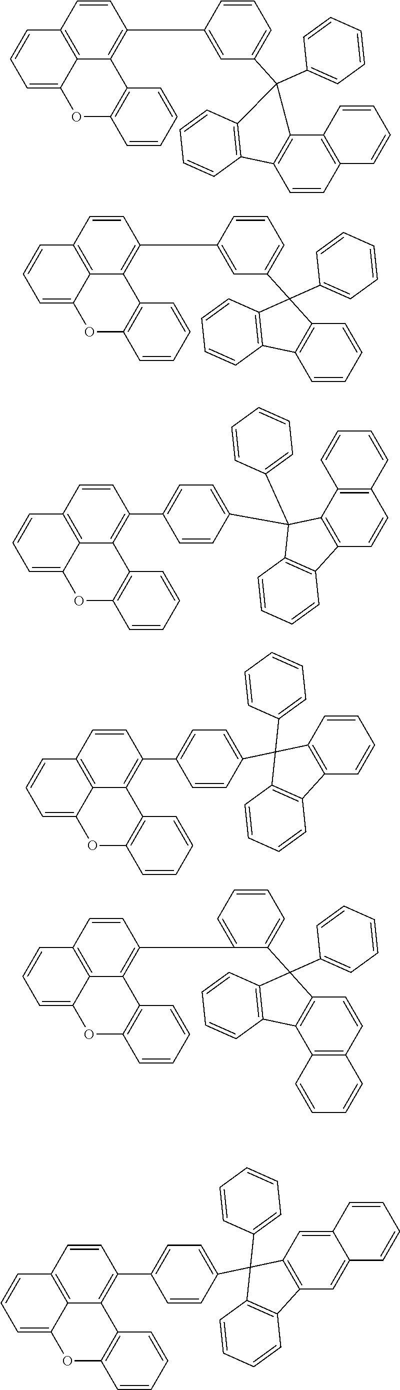

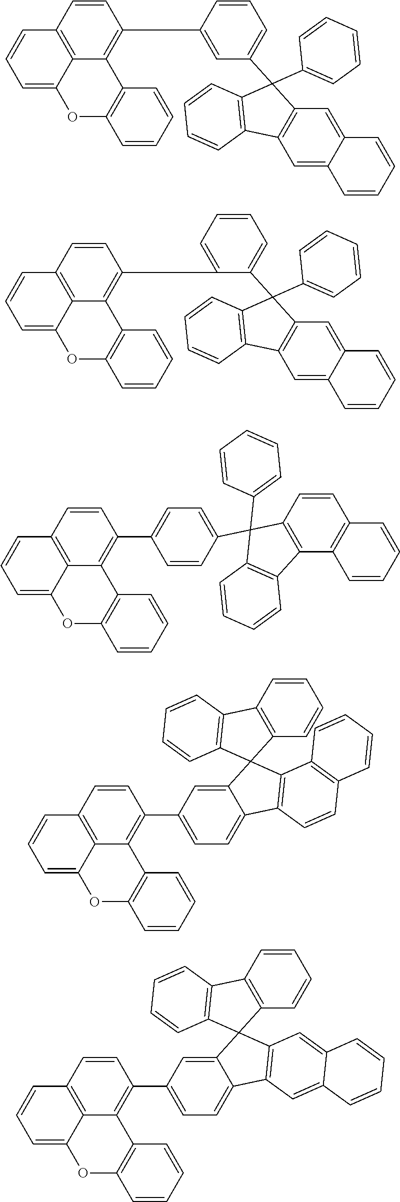

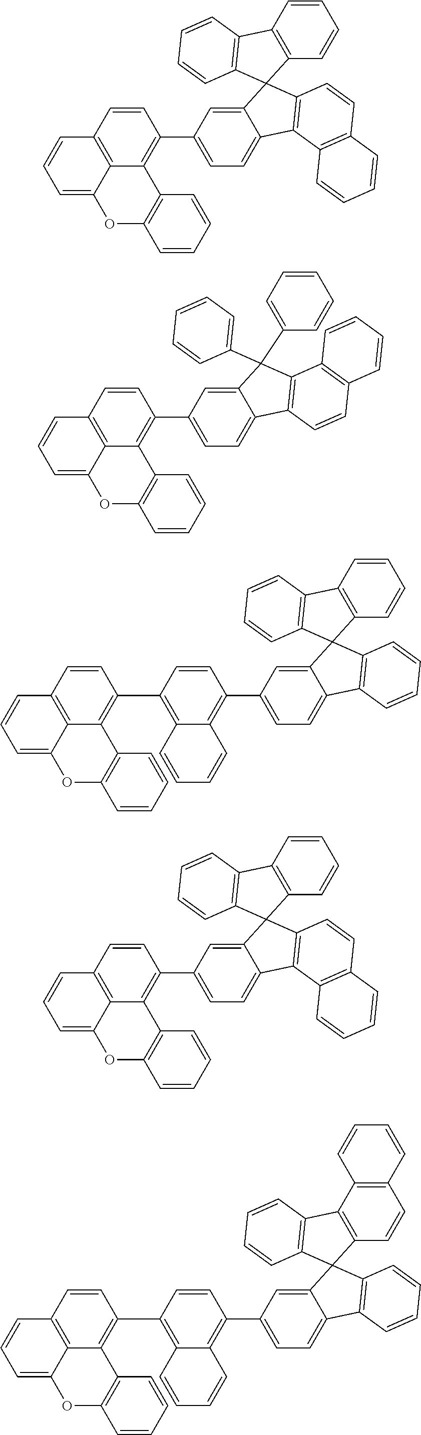

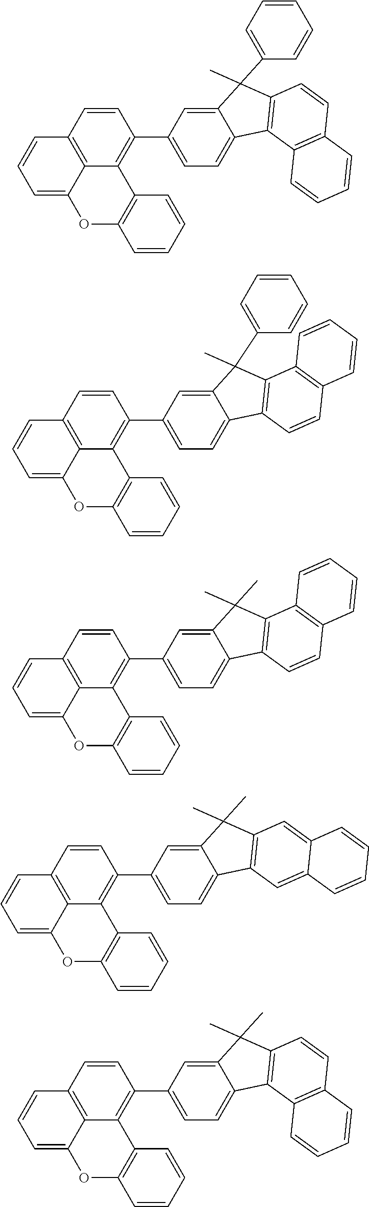

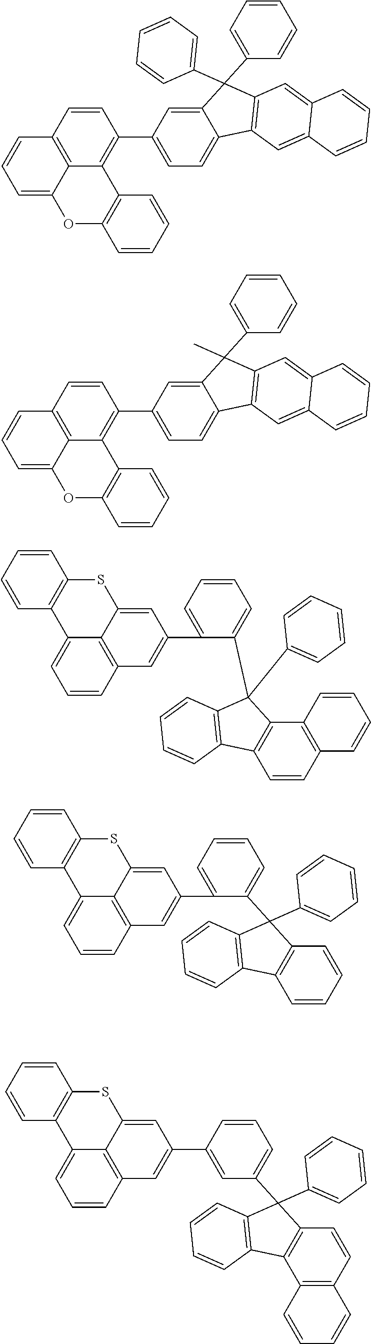 [SMILES: c1ccc(C2(c3ccccc3)c3cc(-c4ccc5cccc6c5c4-c4ccccc4O6)ccc3-c3cc4ccccc4cc32)cc1 ; CC1(c2ccccc2)c2cc(-c3ccc4cccc5c4c3-c3ccccc3O5)ccc2-c2cc3ccccc3cc21 ; c1ccc(C2(c3ccccc3-c3cc4c5c(cccc5c3)-c3ccccc3S4)c3ccccc3-c3ccc4ccccc4c32)cc1 ; c1ccc(C2(c3ccccc3-c3cc4c5c(cccc5c3)-c3ccccc3S4)c3ccccc3-c3ccccc32)cc1 ; c1ccc(C2(c3cccc(-c4cc5c6c(cccc6c4)-c4ccccc4S5)c3)c3ccccc3-c3c2ccc2ccccc32)cc1]

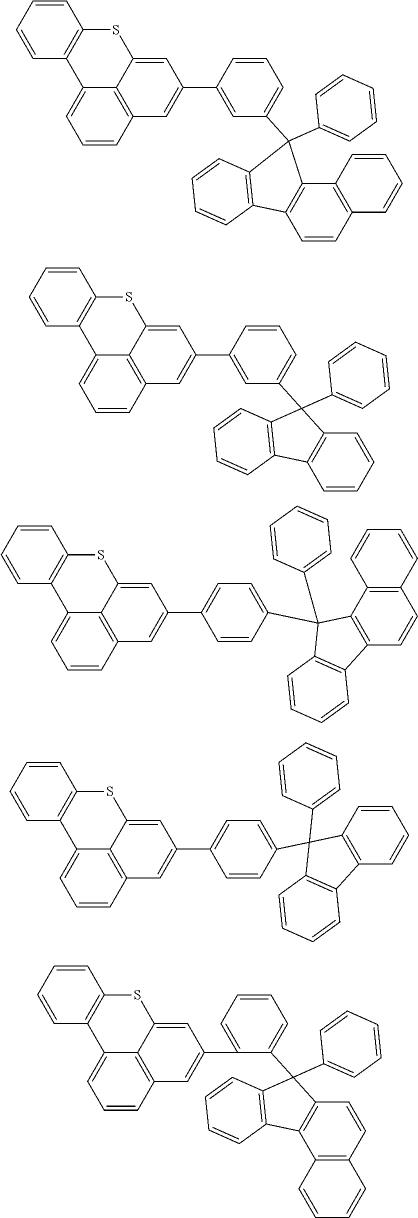

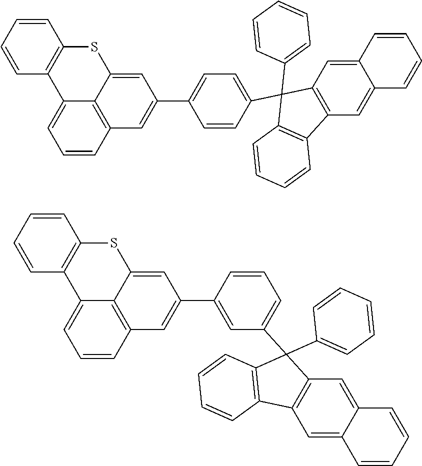 [SMILES: c1ccc(C2(c3ccc(-c4cc5c6c(cccc6c4)-c4ccccc4S5)cc3)c3ccccc3-c3cc4ccccc4cc32)cc1 ; c1ccc(C2(c3cccc(-c4cc5c6c(cccc6c4)-c4ccccc4S5)c3)c3ccccc3-c3cc4ccccc4cc32)cc1]

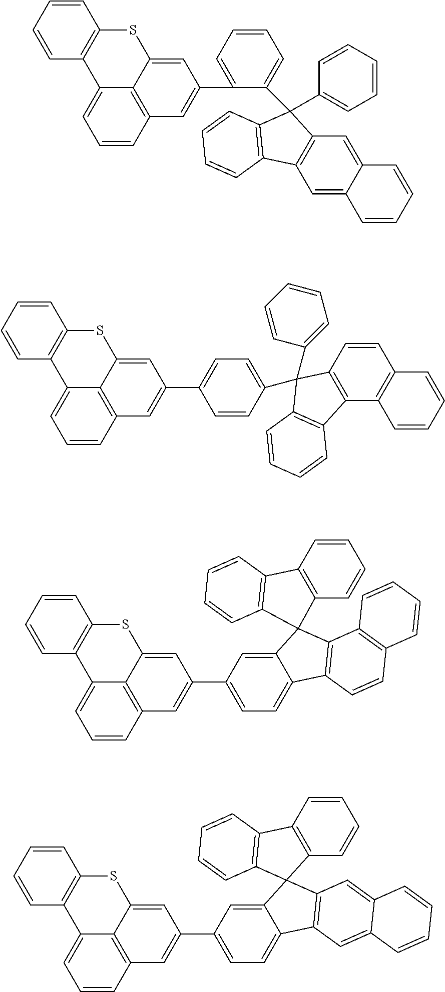 [SMILES: c1ccc(C2(c3ccccc3-c3cc4c5c(cccc5c3)-c3ccccc3S4)c3ccccc3-c3cc4ccccc4cc32)cc1 ; c1ccc(C2(c3ccc(-c4cc5c6c(cccc6c4)-c4ccccc4S5)cc3)c3ccccc3-c3c2ccc2ccccc32)cc1 ; c1ccc2c(c1)Sc1cc(-c3ccc4c(c3)C3(c5ccccc5-c5ccccc53)c3c-4ccc4ccccc34)cc3cccc-2c13 ; c1ccc2c(c1)Sc1cc(-c3ccc4c(c3)C3(c5ccccc5-c5ccccc53)c3cc5ccccc5cc3-4)cc3cccc-2c13]

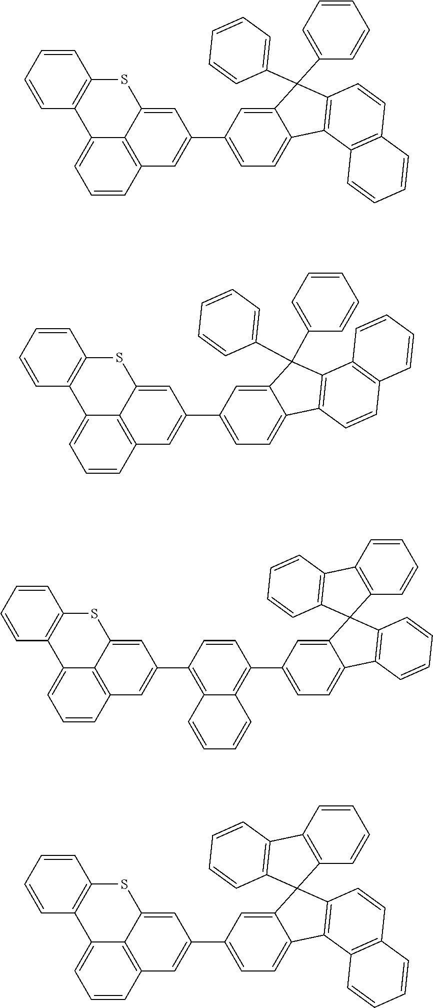 [SMILES: c1ccc(C2(c3ccccc3)c3cc(-c4cc5c6c(cccc6c4)-c4ccccc4S5)ccc3-c3c2ccc2ccccc32)cc1 ; c1ccc(C2(c3ccccc3)c3cc(-c4cc5c6c(cccc6c4)-c4ccccc4S5)ccc3-c3ccc4ccccc4c32)cc1 ; c1ccc2c(c1)Sc1cc(-c3ccc(-c4ccc5c(c4)C4(c6ccccc6-c6ccccc64)c4ccccc4-5)c4ccccc34)cc3cccc-2c13 ; c1ccc2c(c1)Sc1cc(-c3ccc4c(c3)C3(c5ccccc5-c5ccccc53)c3ccc5ccccc5c3-4)cc3cccc-2c13]

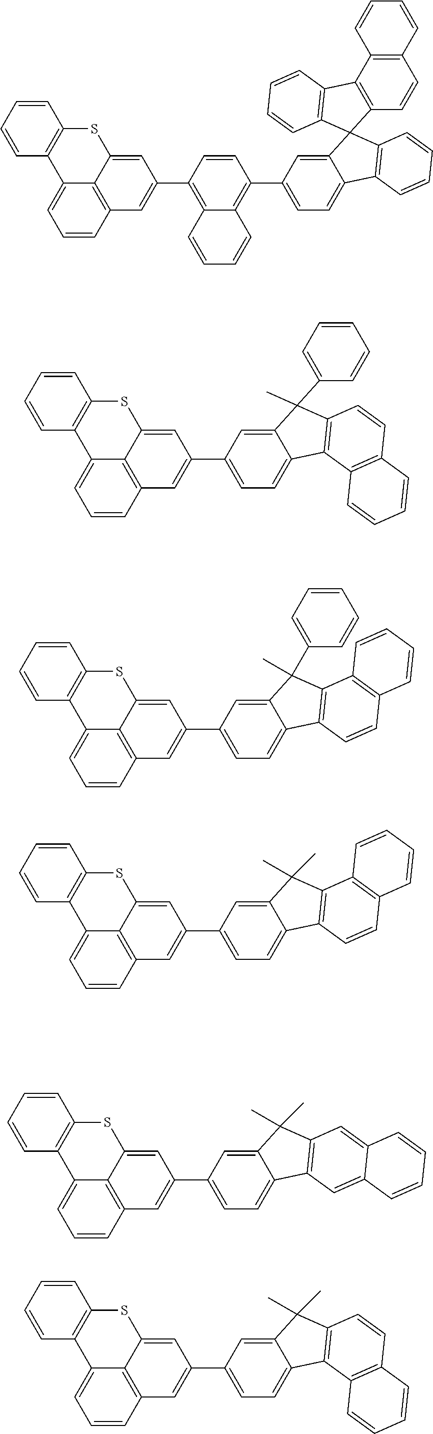

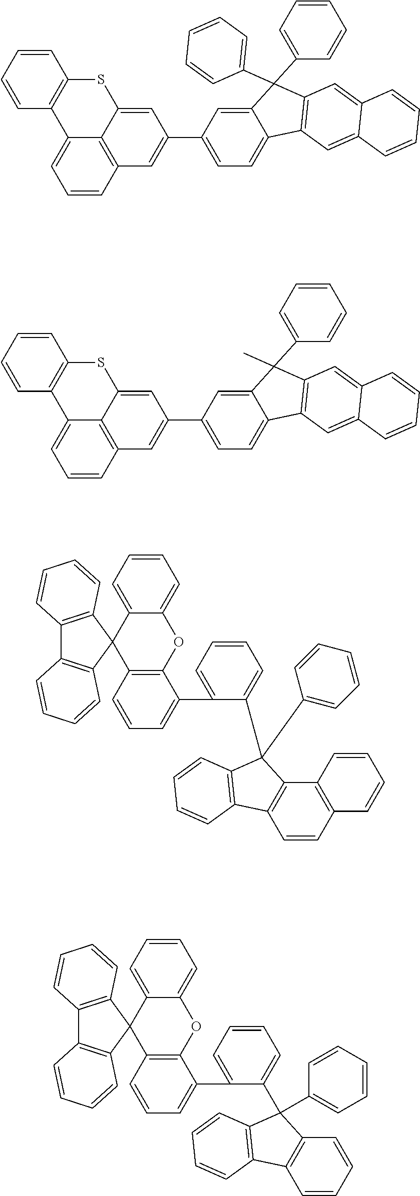 [SMILES: c1ccc(C2(c3ccccc3)c3cc(-c4cc5c6c(cccc6c4)-c4ccccc4S5)ccc3-c3cc4ccccc4cc32)cc1 ; CC1(c2ccccc2)c2cc(-c3cc4c5c(cccc5c3)-c3ccccc3S4)ccc2-c2cc3ccccc3cc21 ; c1ccc(C2(c3ccccc3-c3cccc4c3Oc3ccccc3C43c4ccccc4-c4ccccc43)c3ccccc3-c3ccc4ccccc4c32)cc1 ; c1ccc(C2(c3ccccc3-c3cccc4c3Oc3ccccc3C43c4ccccc4-c4ccccc43)c3ccccc3-c3ccccc32)cc1]

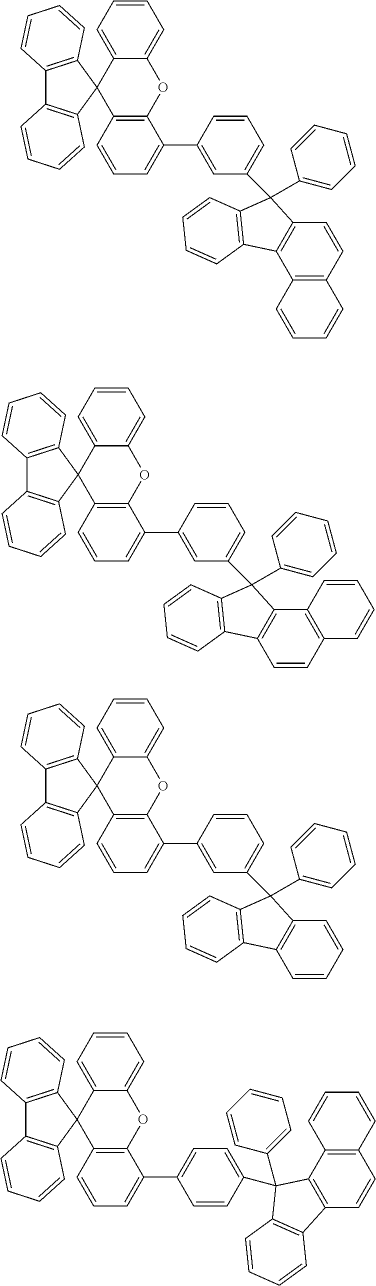 [SMILES: c1ccc(C2(c3cccc(-c4cccc5c4Oc4ccccc4C54c5ccccc5-c5ccccc54)c3)c3ccccc3-c3c2ccc2ccccc32)cc1 ; c1ccc(C2(c3cccc(-c4cccc5c4Oc4ccccc4C54c5ccccc5-c5ccccc54)c3)c3ccccc3-c3ccc4ccccc4c32)cc1 ; c1ccc(C2(c3cccc(-c4cccc5c4Oc4ccccc4C54c5ccccc5-c5ccccc54)c3)c3ccccc3-c3ccccc32)cc1 ; c1ccc(C2(c3ccc(-c4cccc5c4Oc4ccccc4C54c5ccccc5-c5ccccc54)cc3)c3ccccc3-c3ccc4ccccc4c32)cc1]

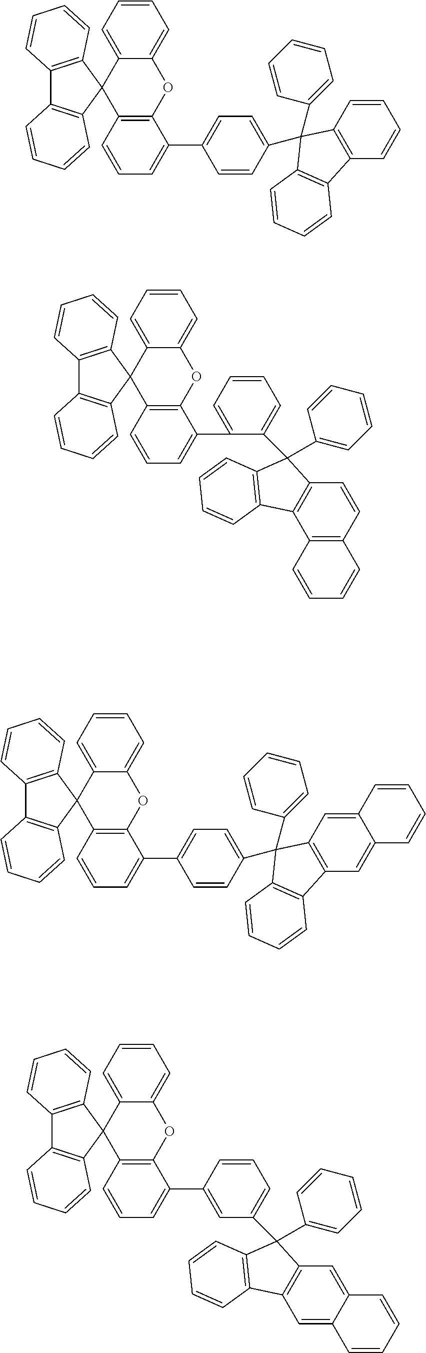 [SMILES: c1ccc(C2(c3ccc(-c4cccc5c4Oc4ccccc4C54c5ccccc5-c5ccccc54)cc3)c3ccccc3-c3ccccc32)cc1 ; c1ccc(C2(c3ccccc3-c3cccc4c3Oc3ccccc3C43c4ccccc4-c4ccccc43)c3ccccc3-c3c2ccc2ccccc32)cc1 ; c1ccc(C2(c3ccc(-c4cccc5c4Oc4ccccc4C54c5ccccc5-c5ccccc54)cc3)c3ccccc3-c3cc4ccccc4cc32)cc1 ; c1ccc(C2(c3cccc(-c4cccc5c4Oc4ccccc4C54c5ccccc5-c5ccccc54)c3)c3ccccc3-c3cc4ccccc4cc32)cc1]

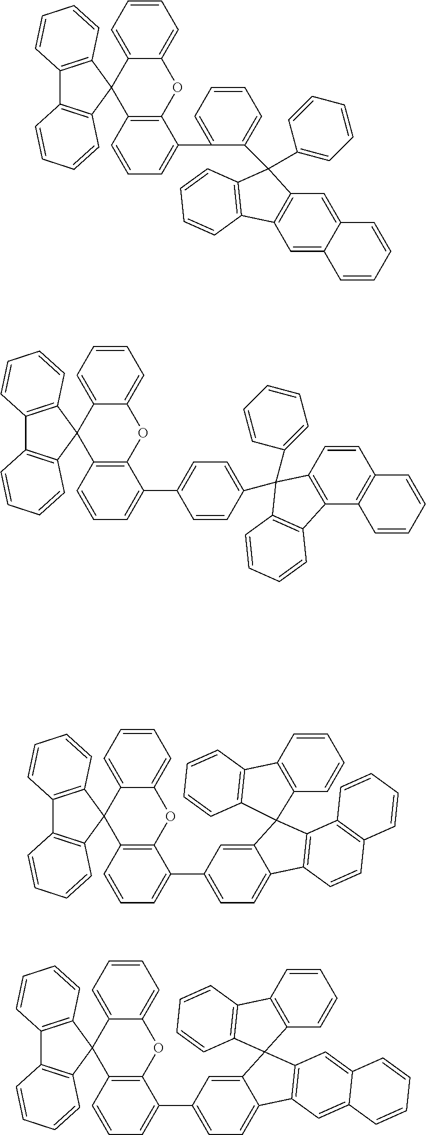 [SMILES: c1ccc(C2(c3ccccc3-c3cccc4c3Oc3ccccc3C43c4ccccc4-c4ccccc43)c3ccccc3-c3cc4ccccc4cc32)cc1 ; c1ccc(C2(c3ccc(-c4cccc5c4Oc4ccccc4C54c5ccccc5-c5ccccc54)cc3)c3ccccc3-c3c2ccc2ccccc32)cc1 ; c1ccc2c(c1)Oc1c(-c3ccc4c(c3)C3(c5ccccc5-c5ccccc53)c3c-4ccc4ccccc34)cccc1C21c2ccccc2-c2ccccc21 ; c1ccc2c(c1)Oc1c(-c3ccc4c(c3)C3(c5ccccc5-c5ccccc53)c3cc5ccccc5cc3-4)cccc1C21c2ccccc2-c2ccccc21]

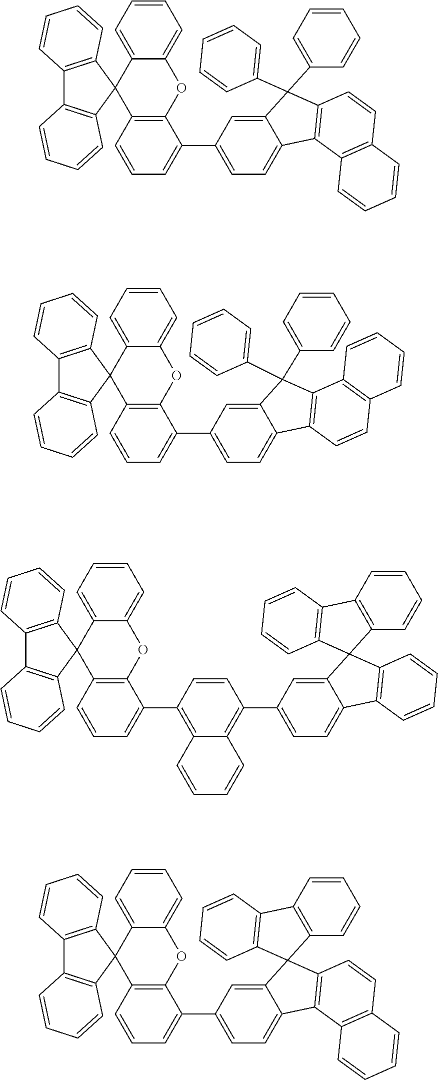 [SMILES: c1ccc(C2(c3ccccc3)c3cc(-c4cccc5c4Oc4ccccc4C54c5ccccc5-c5ccccc54)ccc3-c3c2ccc2ccccc32)cc1 ; c1ccc(C2(c3ccccc3)c3cc(-c4cccc5c4Oc4ccccc4C54c5ccccc5-c5ccccc54)ccc3-c3ccc4ccccc4c32)cc1 ; c1ccc2c(c1)Oc1c(-c3ccc(-c4ccc5c(c4)C4(c6ccccc6-c6ccccc64)c4ccccc4-5)c4ccccc34)cccc1C21c2ccccc2-c2ccccc21 ; c1ccc2c(c1)Oc1c(-c3ccc4c(c3)C3(c5ccccc5-c5ccccc53)c3ccc5ccccc5c3-4)cccc1C21c2ccccc2-c2ccccc21]

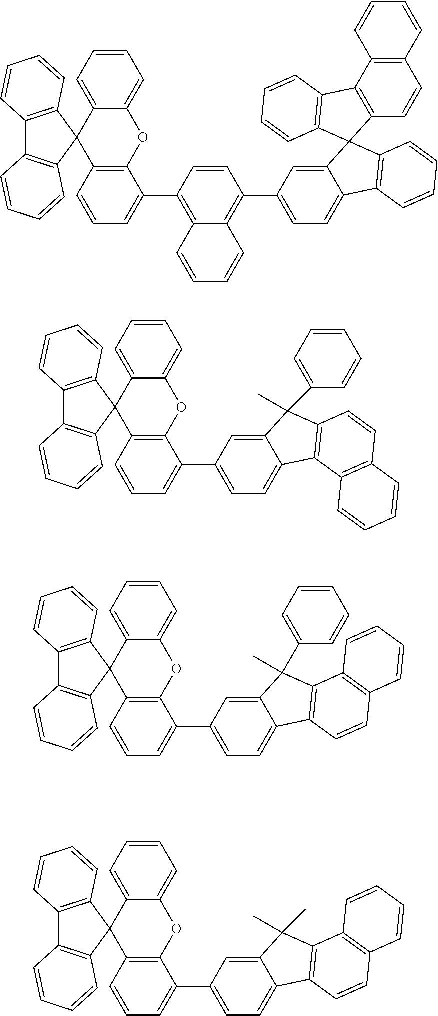 [SMILES: c1ccc2c(c1)Oc1c(-c3ccc(-c4ccc5c(c4)C4(c6ccccc6-5)c5ccccc5-c5c4ccc4ccccc54)c4ccccc34)cccc1C21c2ccccc2-c2ccccc21 ; CC1(c2ccccc2)c2cc(-c3cccc4c3Oc3ccccc3C43c4ccccc4-c4ccccc43)ccc2-c2c1ccc1ccccc21 ; CC1(c2ccccc2)c2cc(-c3cccc4c3Oc3ccccc3C43c4ccccc4-c4ccccc43)ccc2-c2ccc3ccccc3c21 ; CC1(C)c2cc(-c3cccc4c3Oc3ccccc3C43c4ccccc4-c4ccccc43)ccc2-c2ccc3ccccc3c21]

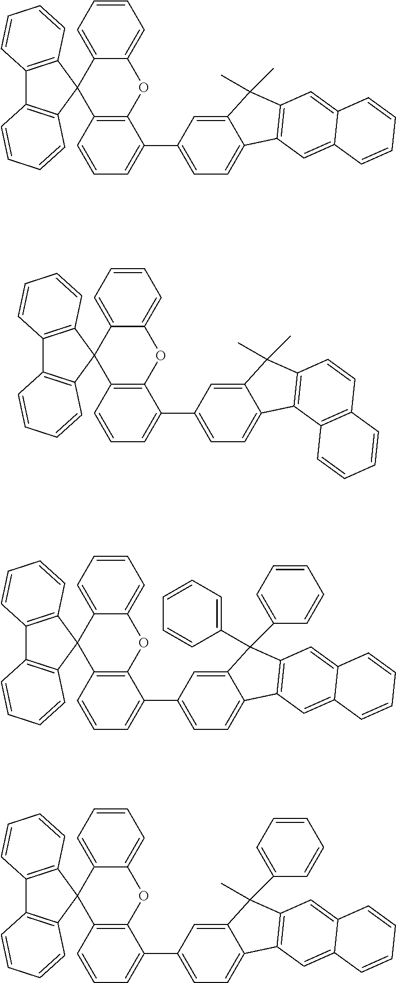 [SMILES: CC1(C)c2cc(-c3cccc4c3Oc3ccccc3C43c4ccccc4-c4ccccc43)ccc2-c2cc3ccccc3cc21 ; CC1(C)c2cc(-c3cccc4c3Oc3ccccc3C43c4ccccc4-c4ccccc43)ccc2-c2c1ccc1ccccc21 ; c1ccc(C2(c3ccccc3)c3cc(-c4cccc5c4Oc4ccccc4C54c5ccccc5-c5ccccc54)ccc3-c3cc4ccccc4cc32)cc1 ; CC1(c2ccccc2)c2cc(-c3cccc4c3Oc3ccccc3C43c4ccccc4-c4ccccc43)ccc2-c2cc3ccccc3cc21]

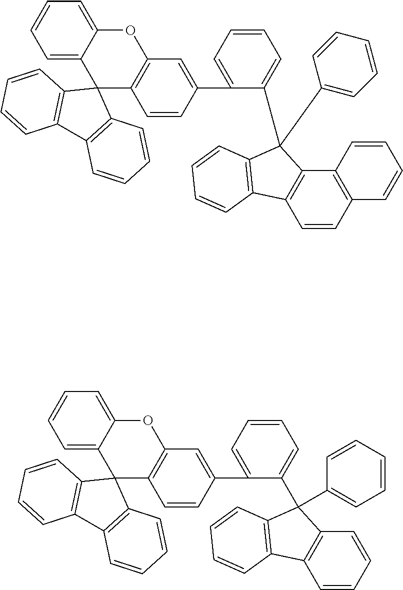 [SMILES: c1ccc(C2(c3ccccc3-c3ccc4c(c3)Oc3ccccc3C43c4ccccc4-c4ccccc43)c3ccccc3-c3ccc4ccccc4c32)cc1 ; c1ccc(C2(c3ccccc3-c3ccc4c(c3)Oc3ccccc3C43c4ccccc4-c4ccccc43)c3ccccc3-c3ccccc32)cc1]

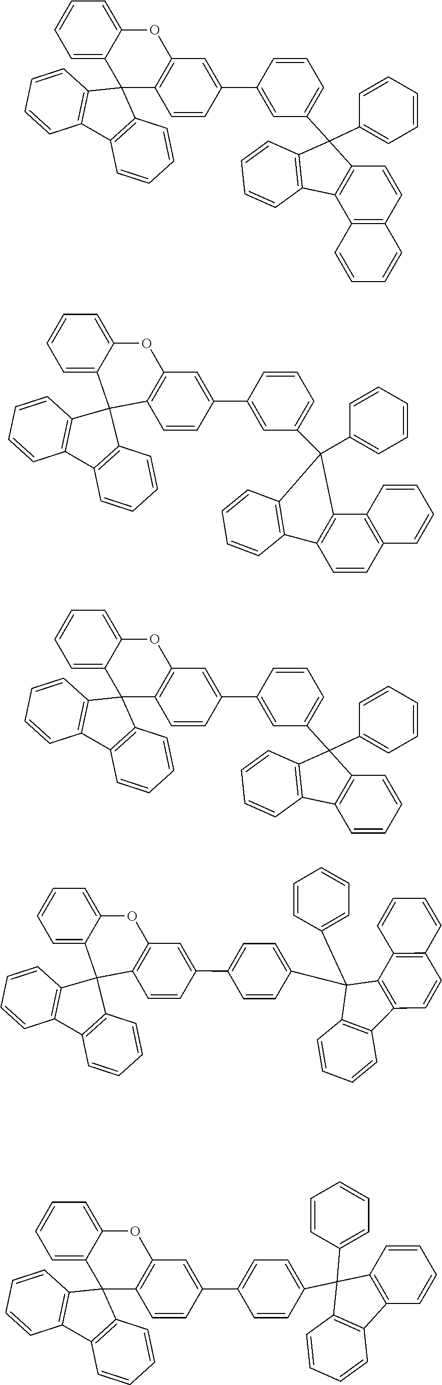

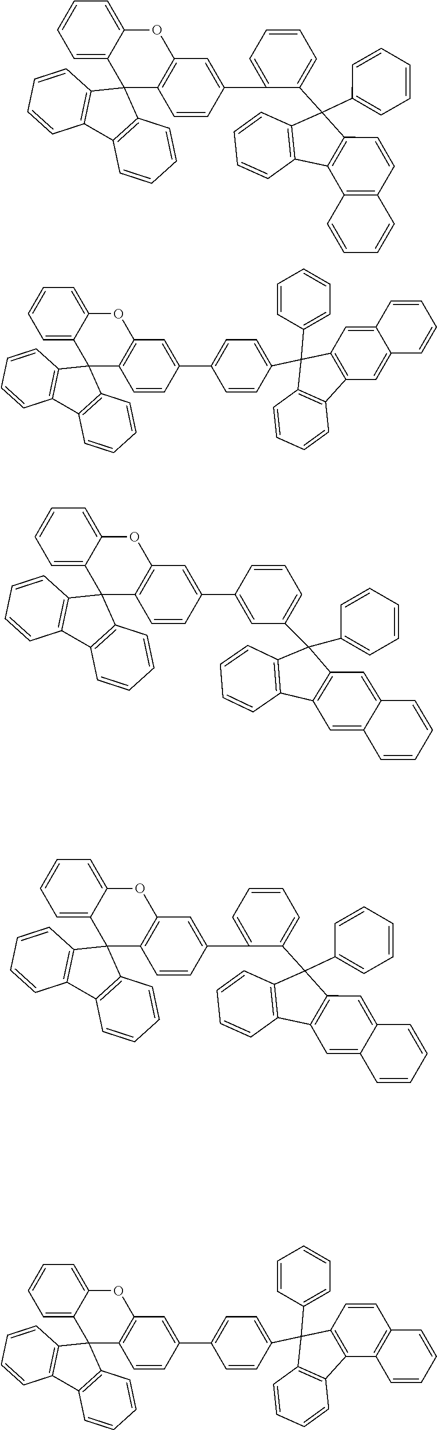

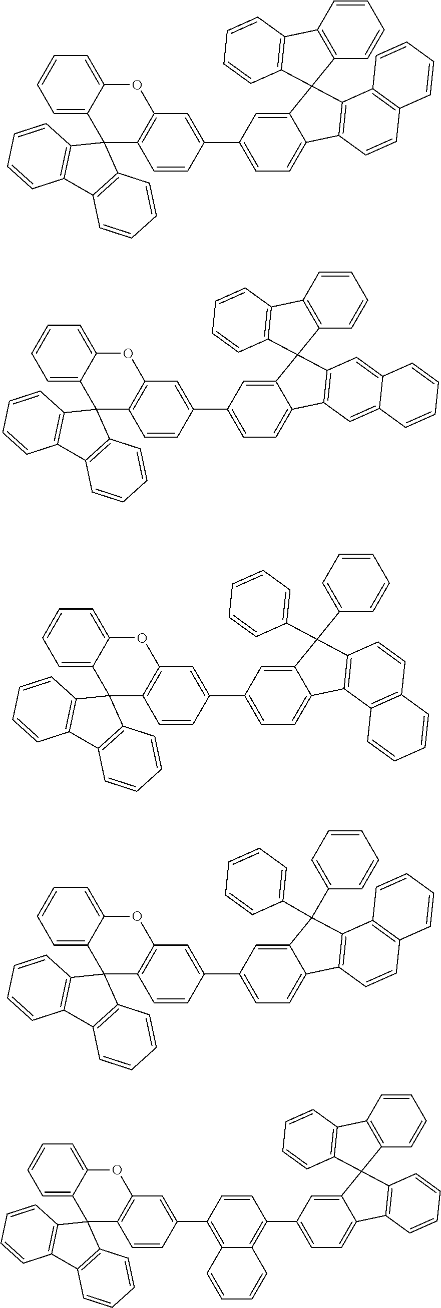 [SMILES: c1ccc2c(c1)Oc1cc(-c3ccc4c(c3)C3(c5ccccc5-c5ccccc53)c3c-4ccc4ccccc34)ccc1C21c2ccccc2-c2ccccc21 ; c1ccc2c(c1)Oc1cc(-c3ccc4c(c3)C3(c5ccccc5-c5ccccc53)c3cc5ccccc5cc3-4)ccc1C21c2ccccc2-c2ccccc21 ; c1ccc(C2(c3ccccc3)c3cc(-c4ccc5c(c4)Oc4ccccc4C54c5ccccc5-c5ccccc54)ccc3-c3c2ccc2ccccc32)cc1 ; c1ccc(C2(c3ccccc3)c3cc(-c4ccc5c(c4)Oc4ccccc4C54c5ccccc5-c5ccccc54)ccc3-c3ccc4ccccc4c32)cc1 ; c1ccc2c(c1)Oc1cc(-c3ccc(-c4ccc5c(c4)C4(c6ccccc6-c6ccccc64)c4ccccc4-5)c4ccccc34)ccc1C21c2ccccc2-c2ccccc21]

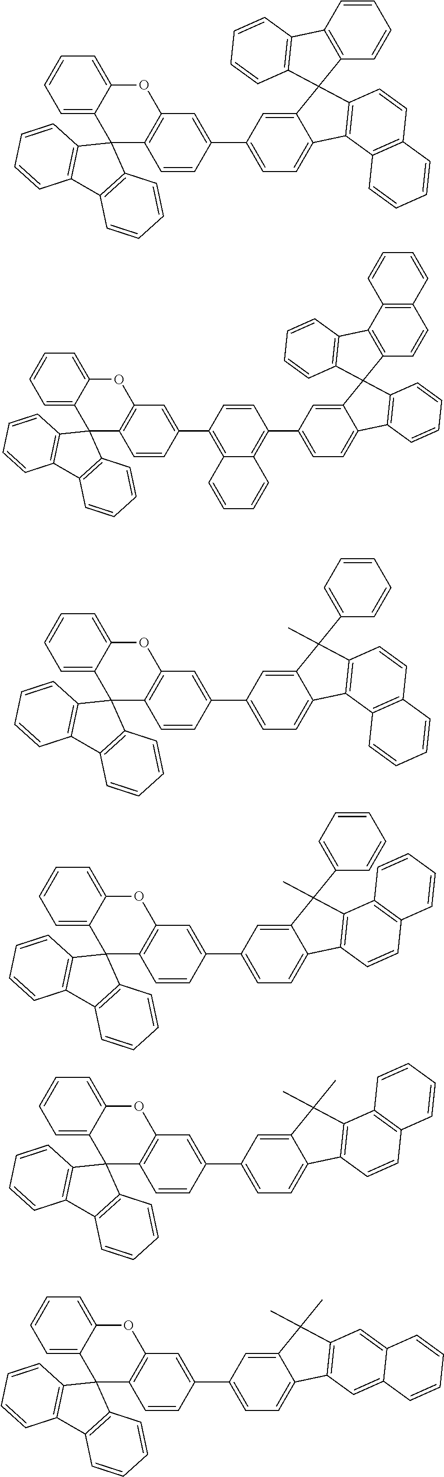

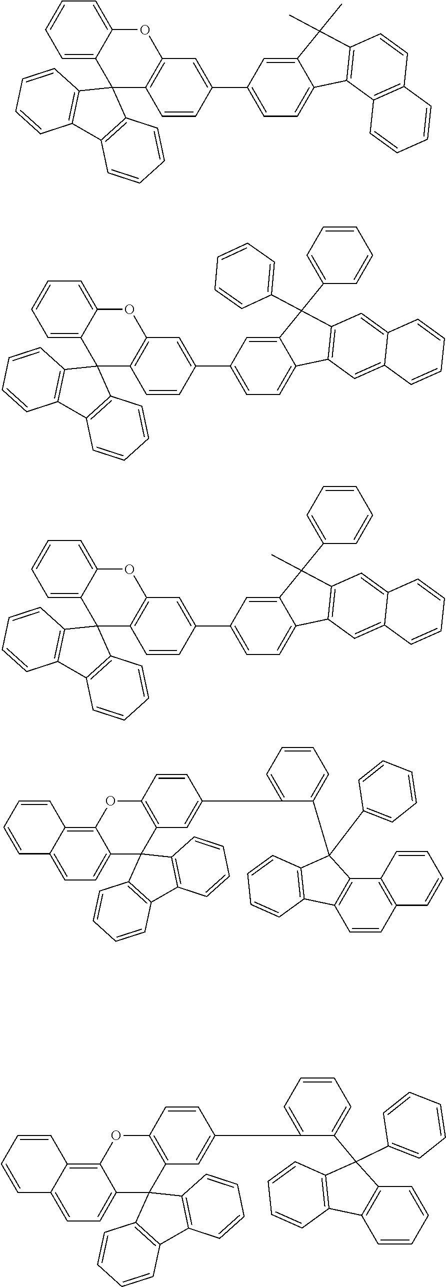 [SMILES: CC1(C)c2cc(-c3ccc4c(c3)Oc3ccccc3C43c4ccccc4-c4ccccc43)ccc2-c2c1ccc1ccccc21 ; c1ccc(C2(c3ccccc3)c3cc(-c4ccc5c(c4)Oc4ccccc4C54c5ccccc5-c5ccccc54)ccc3-c3cc4ccccc4cc32)cc1 ; CC1(c2ccccc2)c2cc(-c3ccc4c(c3)Oc3ccccc3C43c4ccccc4-c4ccccc43)ccc2-c2cc3ccccc3cc21 ; c1ccc(C2(c3ccccc3-c3ccc4c(c3)C3(c5ccccc5-c5ccccc53)c3ccc5ccccc5c3O4)c3ccccc3-c3ccc4ccccc4c32)cc1 ; c1ccc(C2(c3ccccc3-c3ccc4c(c3)C3(c5ccccc5-c5ccccc53)c3ccc5ccccc5c3O4)c3ccccc3-c3ccccc32)cc1]

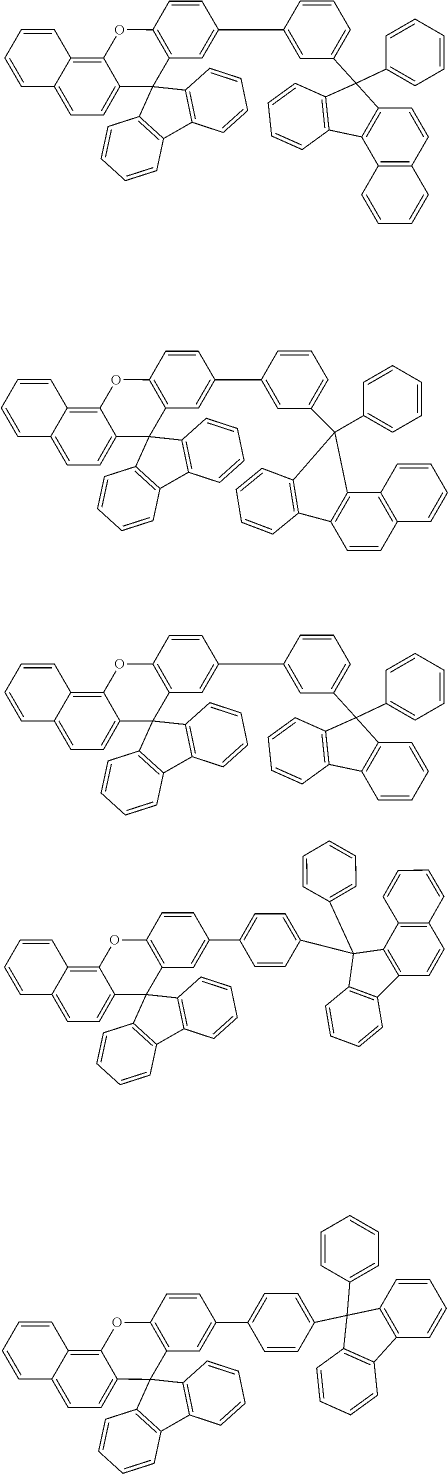 [SMILES: c1ccc(C2(c3cccc(-c4ccc5c(c4)C4(c6ccccc6-c6ccccc64)c4ccc6ccccc6c4O5)c3)c3ccccc3-c3c2ccc2ccccc32)cc1 ; c1ccc(C2(c3cccc(-c4ccc5c(c4)C4(c6ccccc6-c6ccccc64)c4ccc6ccccc6c4O5)c3)c3ccccc3-c3ccc4ccccc4c32)cc1 ; c1ccc(C2(c3cccc(-c4ccc5c(c4)C4(c6ccccc6-c6ccccc64)c4ccc6ccccc6c4O5)c3)c3ccccc3-c3ccccc32)cc1 ; c1ccc(C2(c3ccc(-c4ccc5c(c4)C4(c6ccccc6-c6ccccc64)c4ccc6ccccc6c4O5)cc3)c3ccccc3-c3ccc4ccccc4c32)cc1 ; c1ccc(C2(c3ccc(-c4ccc5c(c4)C4(c6ccccc6-c6ccccc64)c4ccc6ccccc6c4O5)cc3)c3ccccc3-c3ccccc32)cc1]

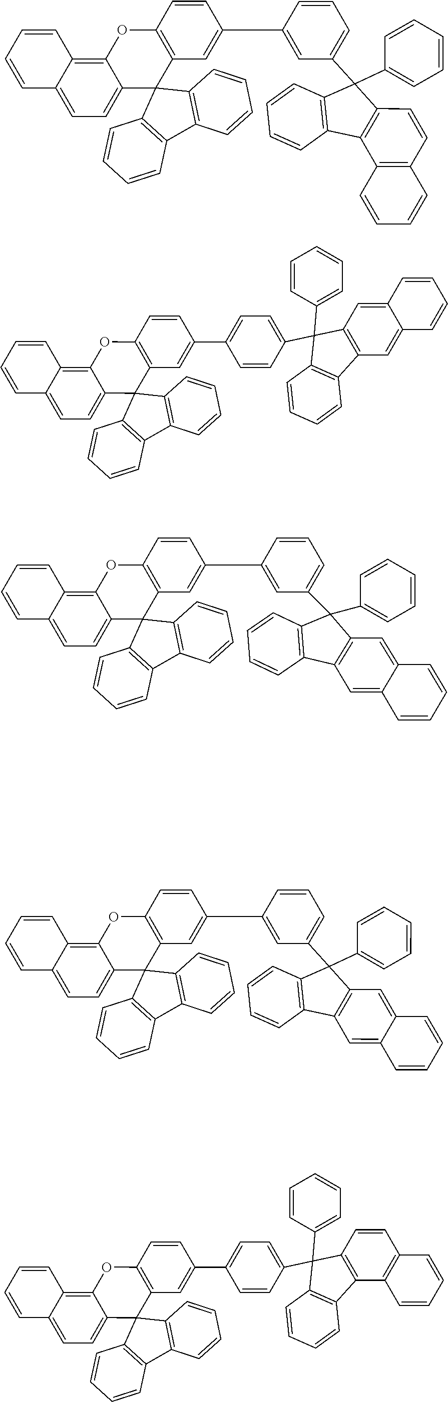

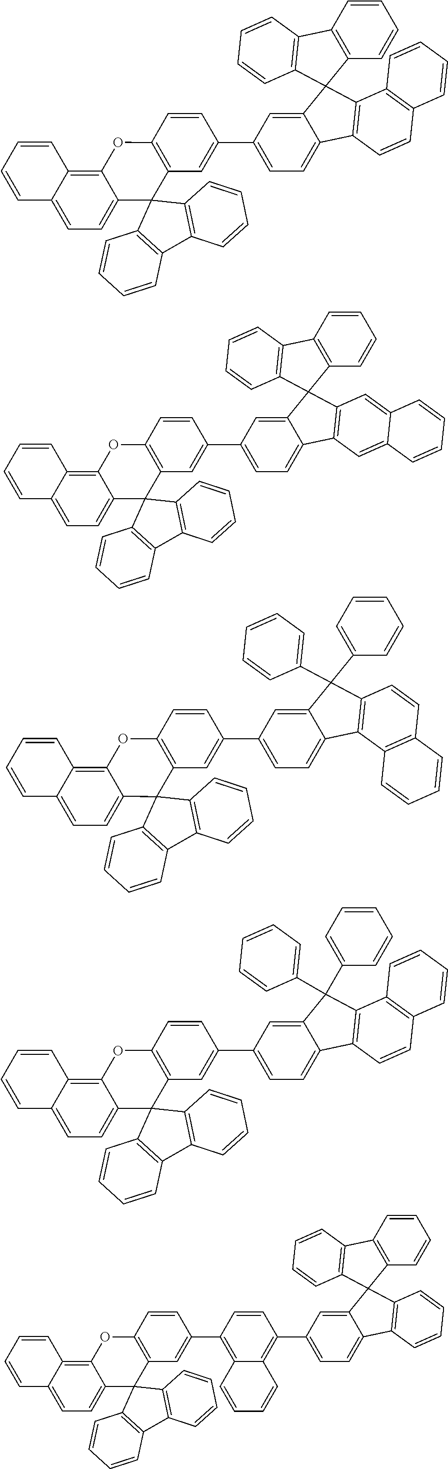

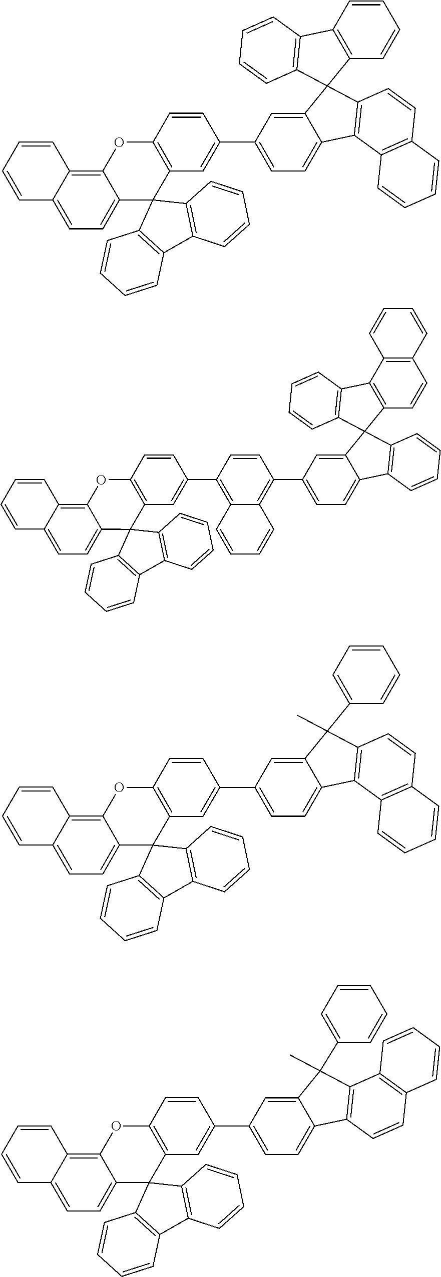 [SMILES: c1ccc2c(c1)-c1ccccc1C21c2cc(-c3ccc4c(c3)C3(c5ccccc5-c5ccccc53)c3ccc5ccccc5c3-4)ccc2Oc2c1ccc1ccccc21 ; c1ccc2c(c1)-c1ccccc1C21c2cc(-c3ccc(-c4ccc5c(c4)C4(c6ccccc6-5)c5ccccc5-c5c4ccc4ccccc54)c4ccccc34)ccc2Oc2c1ccc1ccccc21 ; CC1(c2ccccc2)c2cc(-c3ccc4c(c3)C3(c5ccccc5-c5ccccc53)c3ccc5ccccc5c3O4)ccc2-c2c1ccc1ccccc21 ; CC1(c2ccccc2)c2cc(-c3ccc4c(c3)C3(c5ccccc5-c5ccccc53)c3ccc5ccccc5c3O4)ccc2-c2ccc3ccccc3c21]

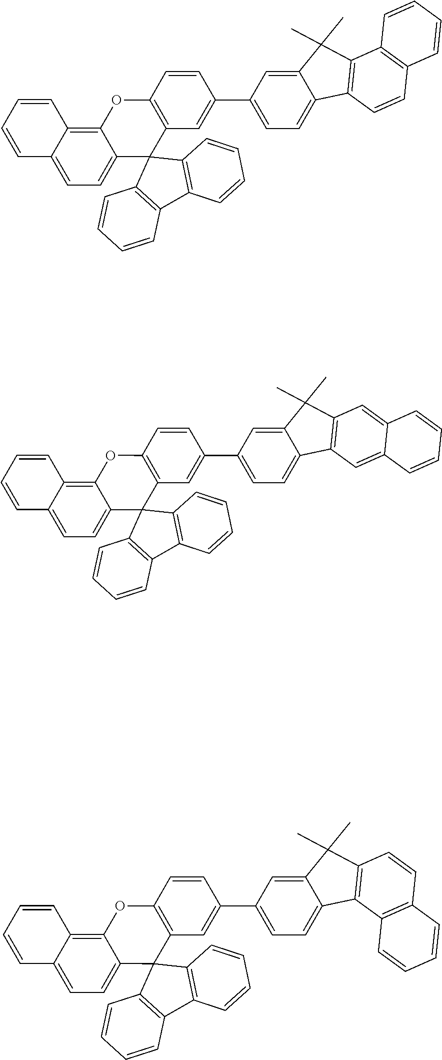 [SMILES: CC1(C)c2cc(-c3ccc4c(c3)C3(c5ccccc5-c5ccccc53)c3ccc5ccccc5c3O4)ccc2-c2ccc3ccccc3c21 ; CC1(C)c2cc(-c3ccc4c(c3)C3(c5ccccc5-c5ccccc53)c3ccc5ccccc5c3O4)ccc2-c2cc3ccccc3cc21 ; CC1(C)c2cc(-c3ccc4c(c3)C3(c5ccccc5-c5ccccc53)c3ccc5ccccc5c3O4)ccc2-c2c1ccc1ccccc21]

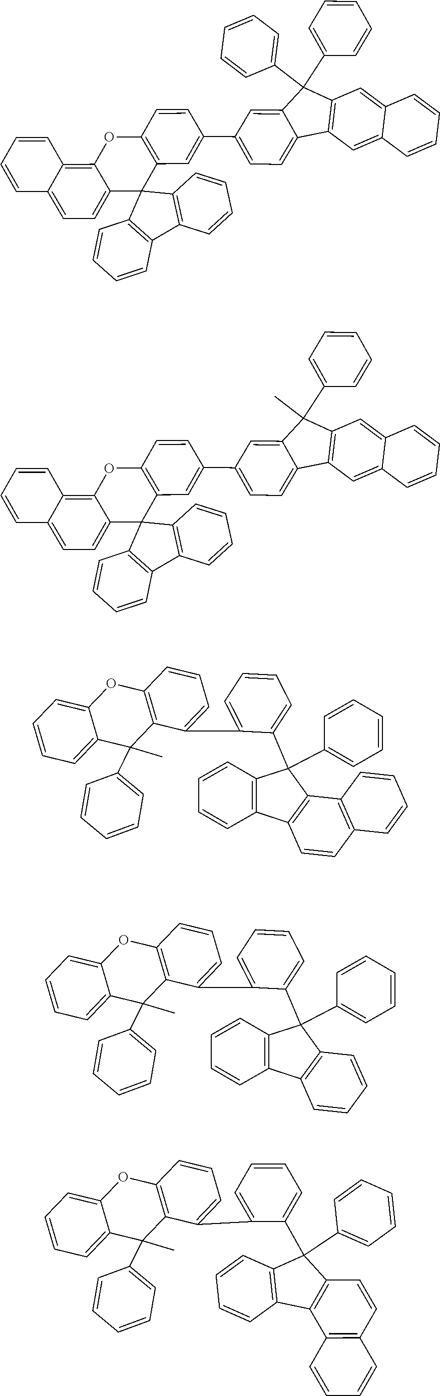 [SMILES: c1ccc(C2(c3ccccc3)c3cc(-c4ccc5c(c4)C4(c6ccccc6-c6ccccc64)c4ccc6ccccc6c4O5)ccc3-c3cc4ccccc4cc32)cc1 ; CC1(c2ccccc2)c2cc(-c3ccc4c(c3)C3(c5ccccc5-c5ccccc53)c3ccc5ccccc5c3O4)ccc2-c2cc3ccccc3cc21 ; CC1(c2ccccc2)c2ccccc2Oc2cccc(-c3ccccc3C3(c4ccccc4)c4ccccc4-c4ccc5ccccc5c43)c21 ; CC1(c2ccccc2)c2ccccc2Oc2cccc(-c3ccccc3C3(c4ccccc4)c4ccccc4-c4ccccc43)c21 ; CC1(c2ccccc2)c2ccccc2Oc2cccc(-c3ccccc3C3(c4ccccc4)c4ccccc4-c4c3ccc3ccccc43)c21]

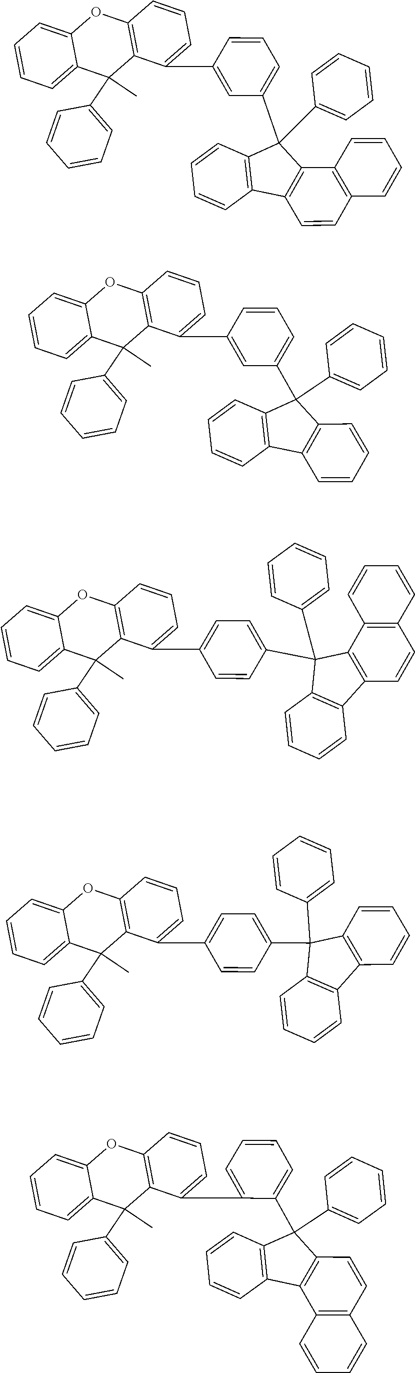 [SMILES: CC1(c2ccccc2)c2ccccc2Oc2cccc(-c3cccc(C4(c5ccccc5)c5ccccc5-c5ccc6ccccc6c54)c3)c21 ; CC1(c2ccccc2)c2ccccc2Oc2cccc(-c3cccc(C4(c5ccccc5)c5ccccc5-c5ccccc54)c3)c21 ; CC1(c2ccccc2)c2ccccc2Oc2cccc(-c3ccc(C4(c5ccccc5)c5ccccc5-c5ccc6ccccc6c54)cc3)c21 ; CC1(c2ccccc2)c2ccccc2Oc2cccc(-c3ccc(C4(c5ccccc5)c5ccccc5-c5ccccc54)cc3)c21 ; CC1(c2ccccc2)c2ccccc2Oc2cccc(-c3ccccc3C3(c4ccccc4)c4ccccc4-c4c3ccc3ccccc43)c21]

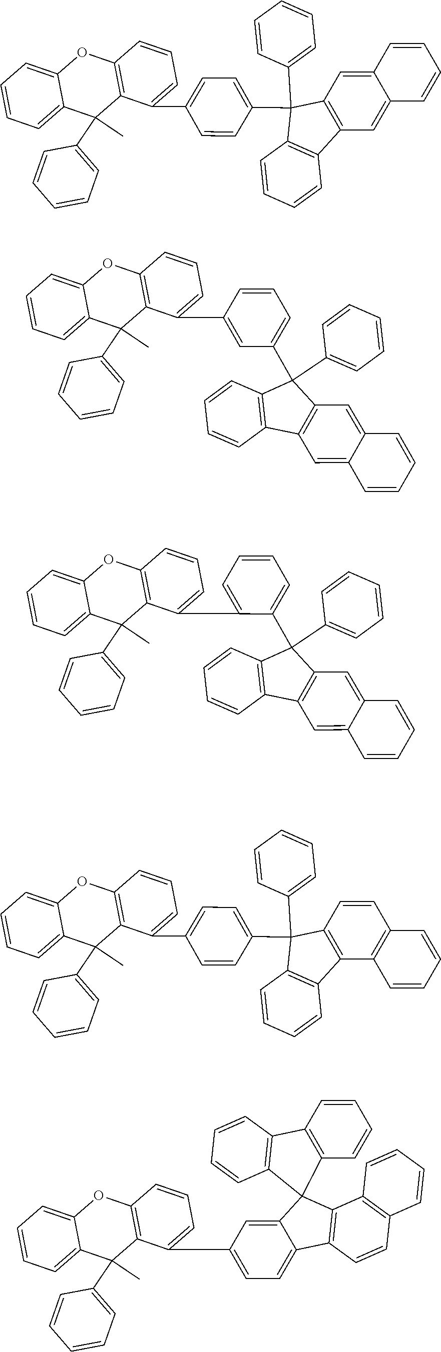 [SMILES: CC1(c2ccccc2)c2ccccc2Oc2cccc(-c3ccc(C4(c5ccccc5)c5ccccc5-c5cc6ccccc6cc54)cc3)c21 ; CC1(c2ccccc2)c2ccccc2Oc2cccc(-c3cccc(C4(c5ccccc5)c5ccccc5-c5cc6ccccc6cc54)c3)c21 ; CC1(c2ccccc2)c2ccccc2Oc2cccc(-c3ccccc3C3(c4ccccc4)c4ccccc4-c4cc5ccccc5cc43)c21 ; CC1(c2ccccc2)c2ccccc2Oc2cccc(-c3ccc(C4(c5ccccc5)c5ccccc5-c5c4ccc4ccccc54)cc3)c21 ; CC1(c2ccccc2)c2ccccc2Oc2cccc(-c3ccc4c(c3)C3(c5ccccc5-c5ccccc53)c3c-4ccc4ccccc34)c21]

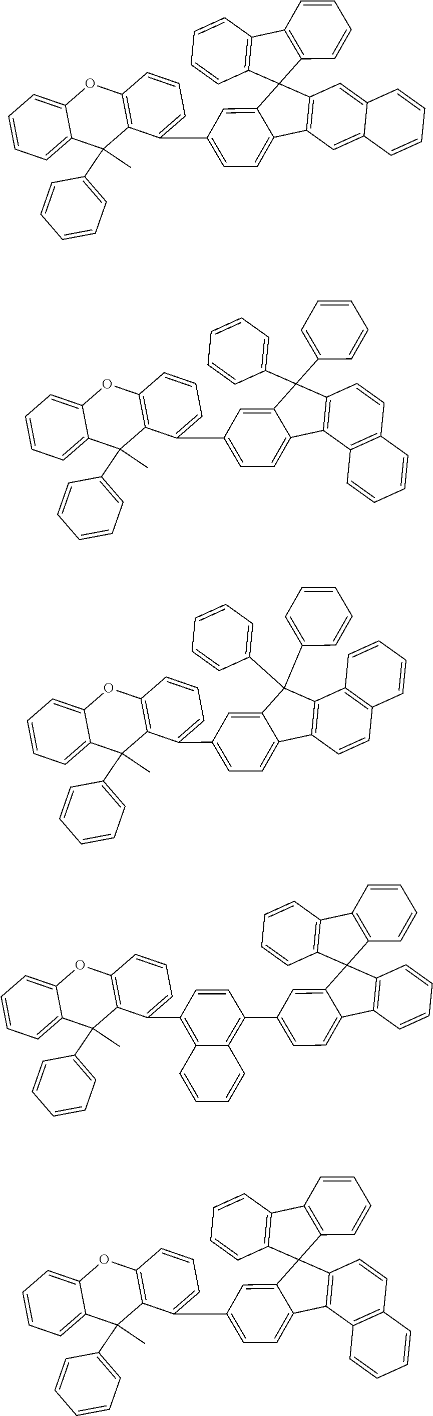 [SMILES: CC1(c2ccccc2)c2ccccc2Oc2cccc(-c3ccc4c(c3)C3(c5ccccc5-c5ccccc53)c3cc5ccccc5cc3-4)c21 ; CC1(c2ccccc2)c2ccccc2Oc2cccc(-c3ccc4c(c3)C(c3ccccc3)(c3ccccc3)c3ccc5ccccc5c3-4)c21 ; CC1(c2ccccc2)c2ccccc2Oc2cccc(-c3ccc4c(c3)C(c3ccccc3)(c3ccccc3)c3c-4ccc4ccccc34)c21 ; CC1(c2ccccc2)c2ccccc2Oc2cccc(-c3ccc(-c4ccc5c(c4)C4(c6ccccc6-c6ccccc64)c4ccccc4-5)c4ccccc34)c21 ; CC1(c2ccccc2)c2ccccc2Oc2cccc(-c3ccc4c(c3)C3(c5ccccc5-c5ccccc53)c3ccc5ccccc5c3-4)c21]

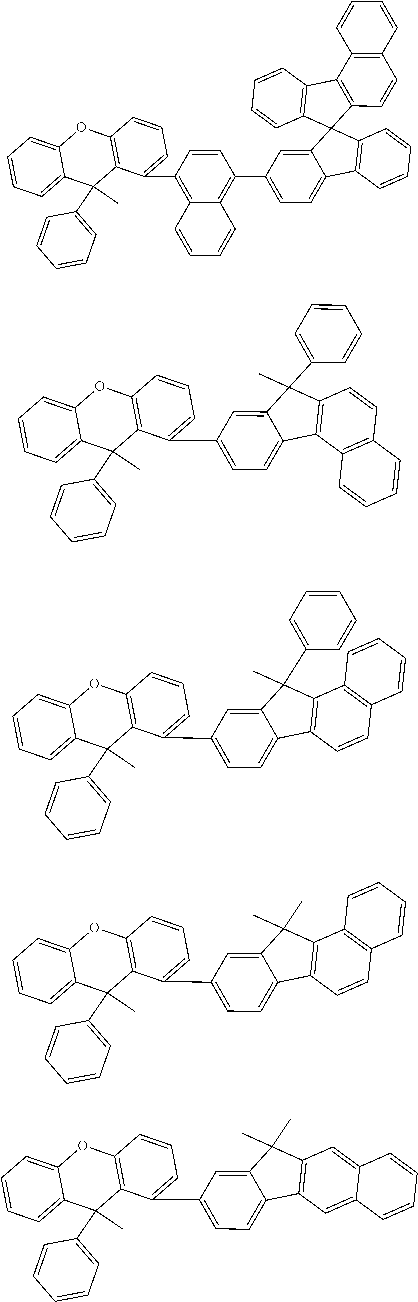 [SMILES: CC1(c2ccccc2)c2ccccc2Oc2cccc(-c3ccc(-c4ccc5c(c4)C4(c6ccccc6-5)c5ccccc5-c5c4ccc4ccccc54)c4ccccc34)c21 ; CC1(c2ccccc2)c2cc(-c3cccc4c3C(C)(c3ccccc3)c3ccccc3O4)ccc2-c2c1ccc1ccccc21 ; CC1(c2ccccc2)c2ccccc2Oc2cccc(-c3ccc4c(c3)C(C)(c3ccccc3)c3c-4ccc4ccccc34)c21 ; CC1(C)c2cc(-c3cccc4c3C(C)(c3ccccc3)c3ccccc3O4)ccc2-c2ccc3ccccc3c21 ; CC1(C)c2cc(-c3cccc4c3C(C)(c3ccccc3)c3ccccc3O4)ccc2-c2cc3ccccc3cc21]

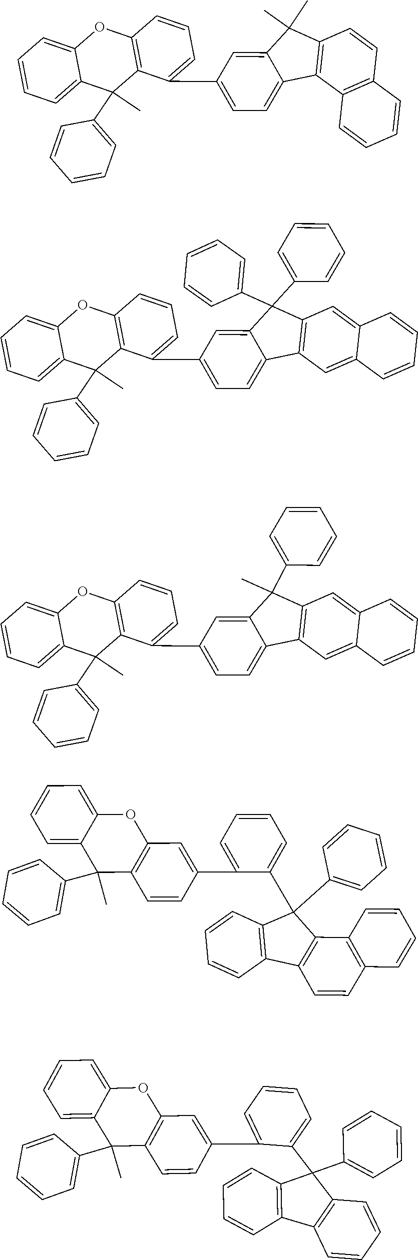

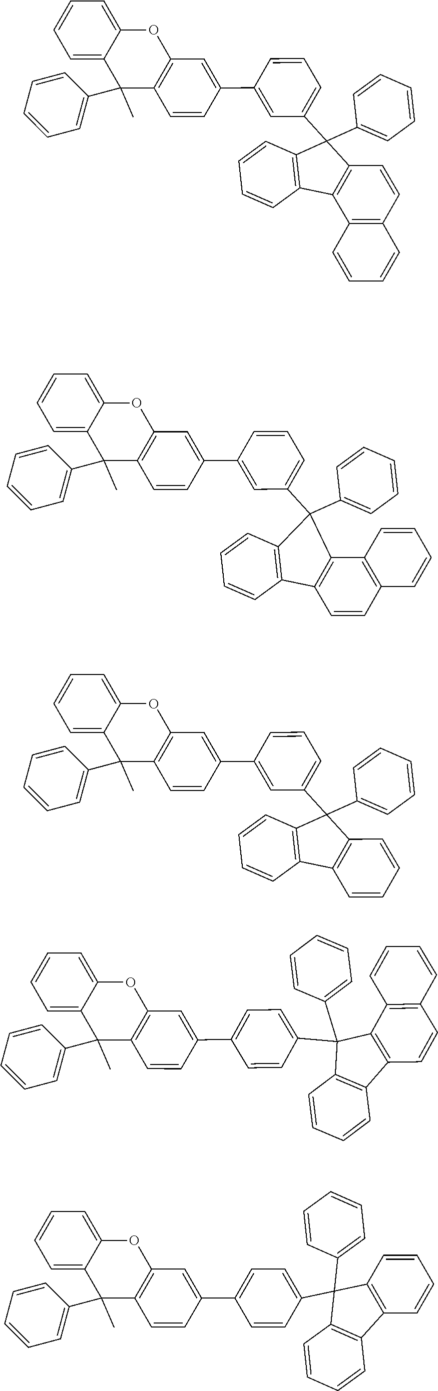 [SMILES: CC1(c2ccccc2)c2ccccc2Oc2cc(-c3cccc(C4(c5ccccc5)c5ccccc5-c5c4ccc4ccccc54)c3)ccc21 ; CC1(c2ccccc2)c2ccccc2Oc2cc(-c3cccc(C4(c5ccccc5)c5ccccc5-c5ccc6ccccc6c54)c3)ccc21 ; CC1(c2ccccc2)c2ccccc2Oc2cc(-c3cccc(C4(c5ccccc5)c5ccccc5-c5ccccc54)c3)ccc21 ; CC1(c2ccccc2)c2ccccc2Oc2cc(-c3ccc(C4(c5ccccc5)c5ccccc5-c5ccc6ccccc6c54)cc3)ccc21 ; CC1(c2ccccc2)c2ccccc2Oc2cc(-c3ccc(C4(c5ccccc5)c5ccccc5-c5ccccc54)cc3)ccc21]

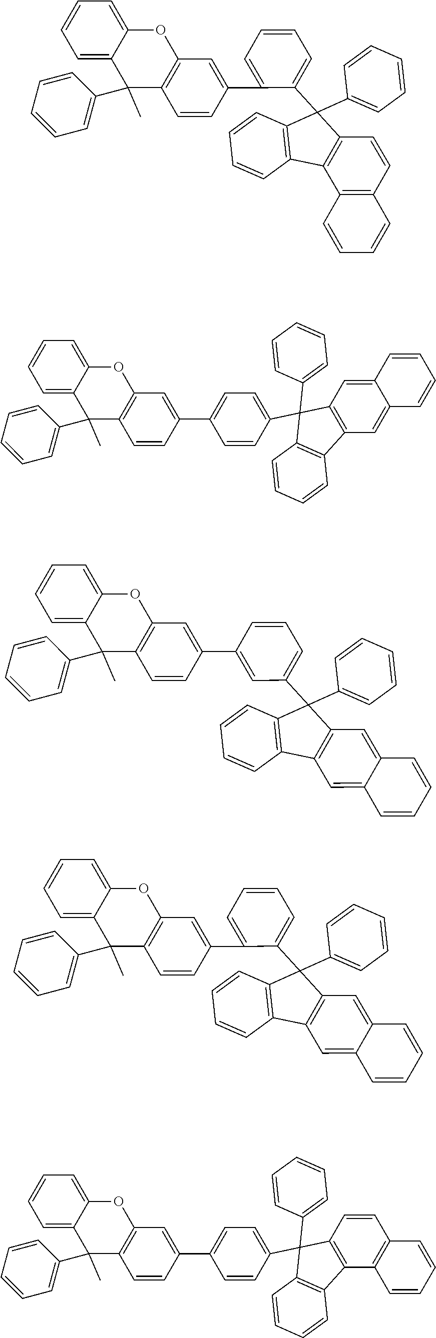 [SMILES: CC1(c2ccccc2)c2ccccc2Oc2cc(-c3ccccc3C3(c4ccccc4)c4ccccc4-c4c3ccc3ccccc43)ccc21 ; CC1(c2ccccc2)c2ccccc2Oc2cc(-c3ccc(C4(c5ccccc5)c5ccccc5-c5cc6ccccc6cc54)cc3)ccc21 ; CC1(c2ccccc2)c2ccccc2Oc2cc(-c3cccc(C4(c5ccccc5)c5ccccc5-c5cc6ccccc6cc54)c3)ccc21 ; CC1(c2ccccc2)c2ccccc2Oc2cc(-c3ccccc3C3(c4ccccc4)c4ccccc4-c4cc5ccccc5cc43)ccc21 ; CC1(c2ccccc2)c2ccccc2Oc2cc(-c3ccc(C4(c5ccccc5)c5ccccc5-c5c4ccc4ccccc54)cc3)ccc21]

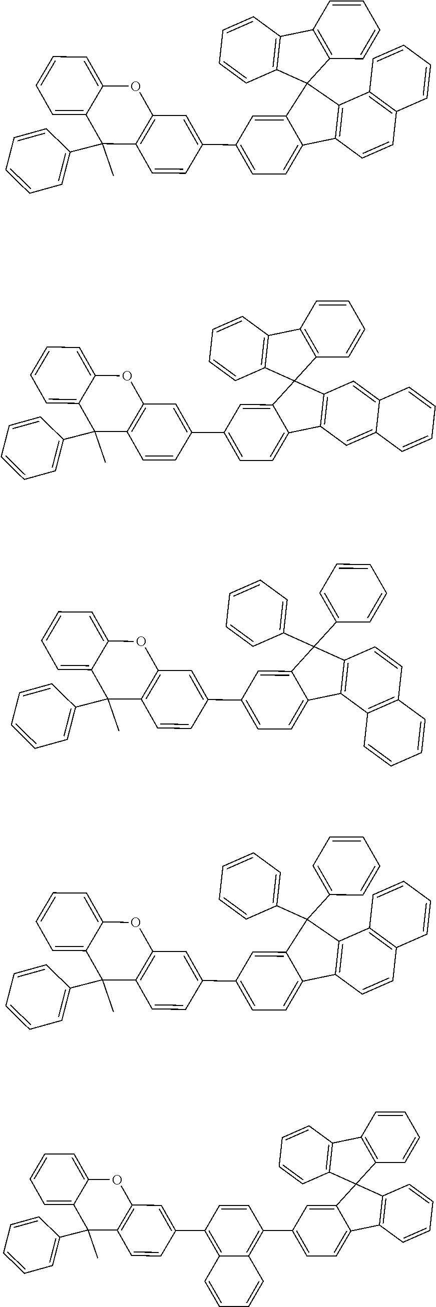 [SMILES: CC1(c2ccccc2)c2ccccc2Oc2cc(-c3ccc4c(c3)C3(c5ccccc5-c5ccccc53)c3c-4ccc4ccccc34)ccc21 ; CC1(c2ccccc2)c2ccccc2Oc2cc(-c3ccc4c(c3)C3(c5ccccc5-c5ccccc53)c3cc5ccccc5cc3-4)ccc21 ; CC1(c2ccccc2)c2ccccc2Oc2cc(-c3ccc4c(c3)C(c3ccccc3)(c3ccccc3)c3ccc5ccccc5c3-4)ccc21 ; CC1(c2ccccc2)c2ccccc2Oc2cc(-c3ccc4c(c3)C(c3ccccc3)(c3ccccc3)c3c-4ccc4ccccc34)ccc21 ; CC1(c2ccccc2)c2ccccc2Oc2cc(-c3ccc(-c4ccc5c(c4)C4(c6ccccc6-c6ccccc64)c4ccccc4-5)c4ccccc34)ccc21]

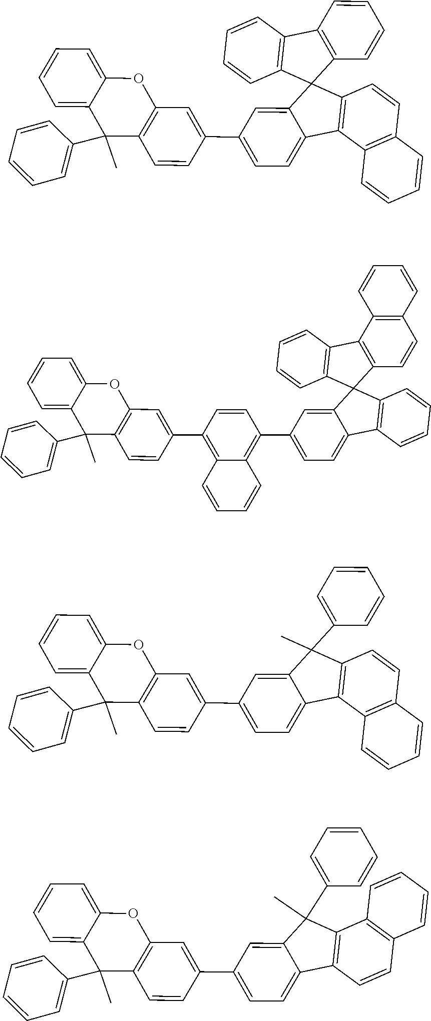 [SMILES: CC1(c2ccccc2)c2ccccc2Oc2cc(-c3ccc4c(c3)C3(c5ccccc5-c5ccccc53)c3ccc5ccccc5c3-4)ccc21 ; CC1(c2ccccc2)c2ccccc2Oc2cc(-c3ccc(-c4ccc5c(c4)C4(c6ccccc6-5)c5ccccc5-c5c4ccc4ccccc54)c4ccccc34)ccc21 ; CC1(c2ccccc2)c2ccccc2Oc2cc(-c3ccc4c(c3)C(C)(c3ccccc3)c3ccc5ccccc5c3-4)ccc21 ; CC1(c2ccccc2)c2ccccc2Oc2cc(-c3ccc4c(c3)C(C)(c3ccccc3)c3c-4ccc4ccccc34)ccc21]

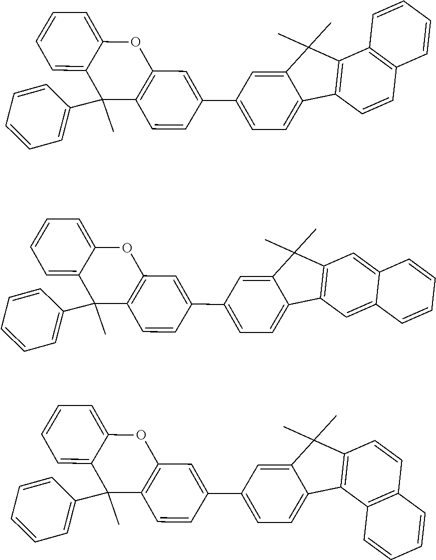 [SMILES: CC1(C)c2cc(-c3ccc4c(c3)Oc3ccccc3C4(C)c3ccccc3)ccc2-c2ccc3ccccc3c21 ; CC1(C)c2cc(-c3ccc4c(c3)Oc3ccccc3C4(C)c3ccccc3)ccc2-c2cc3ccccc3cc21 ; CC1(C)c2cc(-c3ccc4c(c3)Oc3ccccc3C4(C)c3ccccc3)ccc2-c2c1ccc1ccccc21]

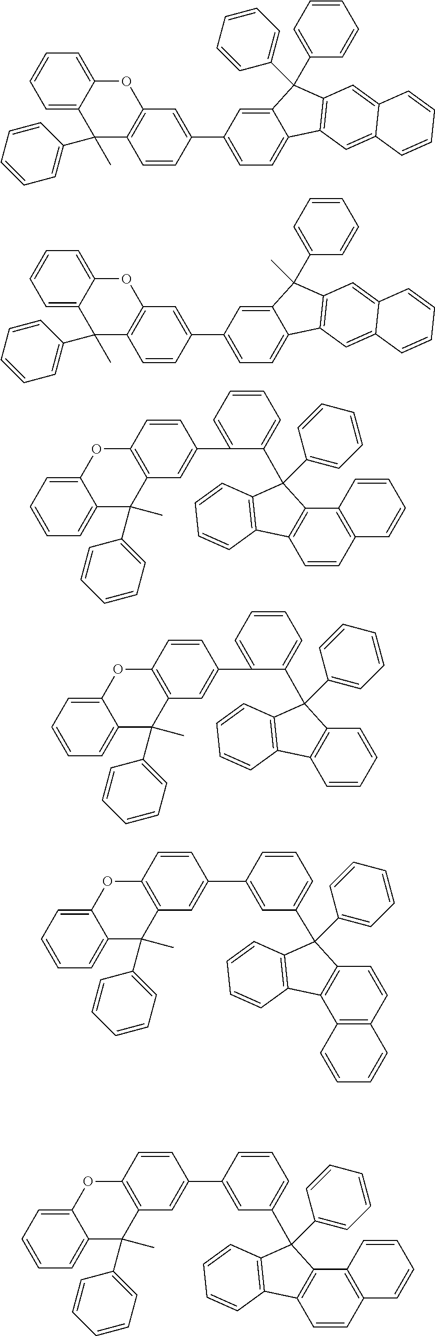

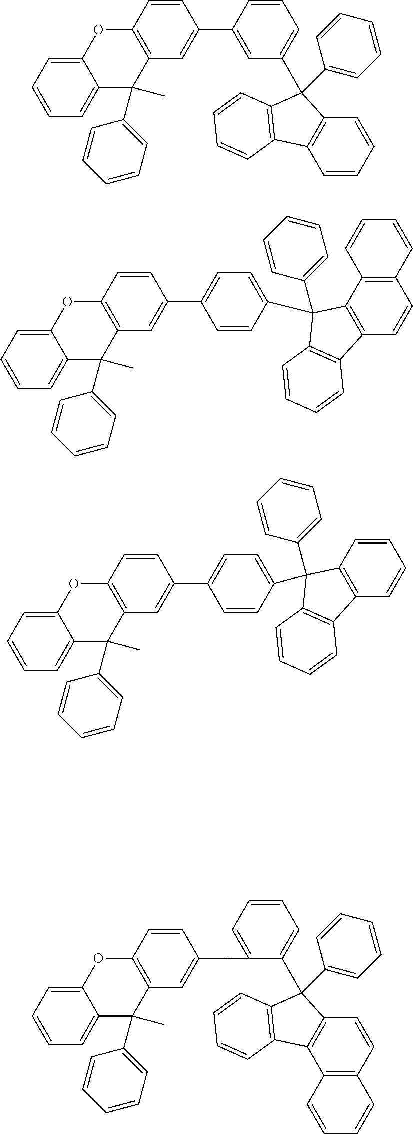

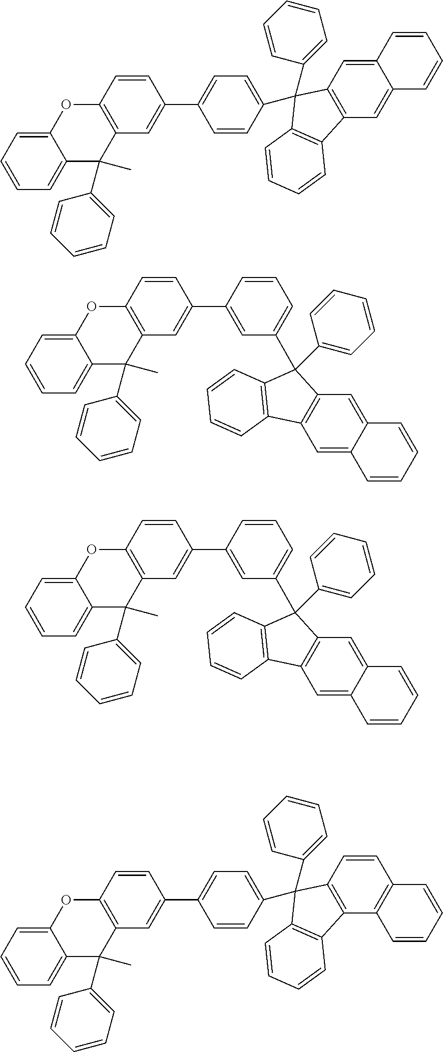 [SMILES: CC1(c2ccccc2)c2ccccc2Oc2ccc(-c3ccc(C4(c5ccccc5)c5ccccc5-c5cc6ccccc6cc54)cc3)cc21 ; CC1(c2ccccc2)c2ccccc2Oc2ccc(-c3cccc(C4(c5ccccc5)c5ccccc5-c5cc6ccccc6cc54)c3)cc21 ; CC1(c2ccccc2)c2ccccc2Oc2ccc(-c3cccc(C4(c5ccccc5)c5ccccc5-c5cc6ccccc6cc54)c3)cc21 ; CC1(c2ccccc2)c2ccccc2Oc2ccc(-c3ccc(C4(c5ccccc5)c5ccccc5-c5c4ccc4ccccc54)cc3)cc21]

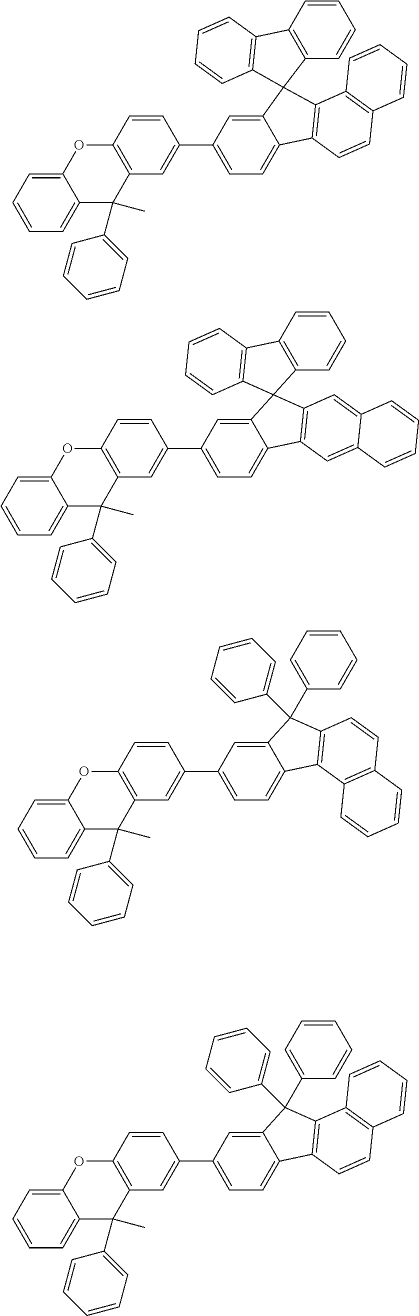 [SMILES: CC1(c2ccccc2)c2ccccc2Oc2ccc(-c3ccc4c(c3)C3(c5ccccc5-c5ccccc53)c3c-4ccc4ccccc34)cc21 ; CC1(c2ccccc2)c2ccccc2Oc2ccc(-c3ccc4c(c3)C3(c5ccccc5-c5ccccc53)c3cc5ccccc5cc3-4)cc21 ; CC1(c2ccccc2)c2ccccc2Oc2ccc(-c3ccc4c(c3)C(c3ccccc3)(c3ccccc3)c3ccc5ccccc5c3-4)cc21 ; CC1(c2ccccc2)c2ccccc2Oc2ccc(-c3ccc4c(c3)C(c3ccccc3)(c3ccccc3)c3c-4ccc4ccccc34)cc21]

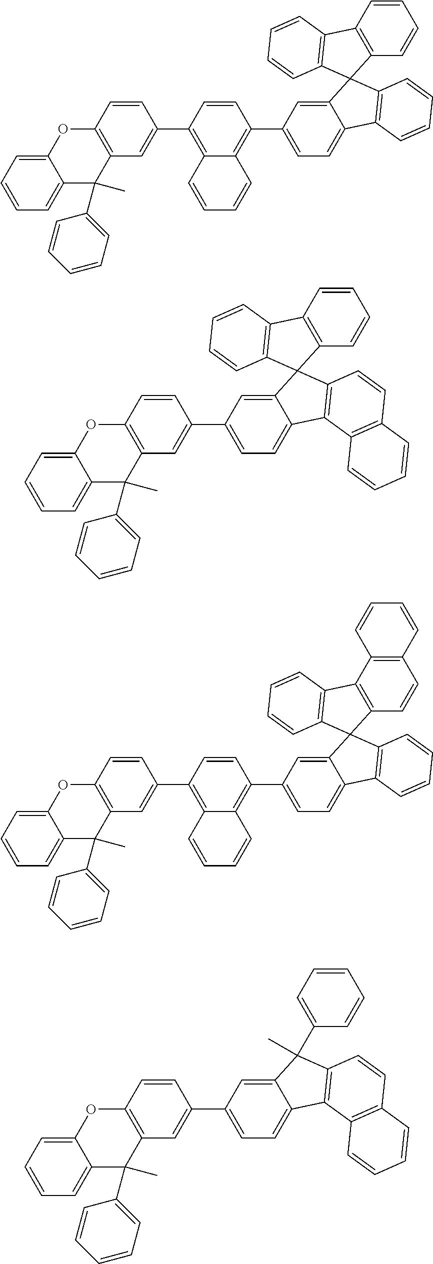 [SMILES: CC1(c2ccccc2)c2ccccc2Oc2ccc(-c3ccc(-c4ccc5c(c4)C4(c6ccccc6-c6ccccc64)c4ccccc4-5)c4ccccc34)cc21 ; CC1(c2ccccc2)c2ccccc2Oc2ccc(-c3ccc4c(c3)C3(c5ccccc5-c5ccccc53)c3ccc5ccccc5c3-4)cc21 ; CC1(c2ccccc2)c2ccccc2Oc2ccc(-c3ccc(-c4ccc5c(c4)C4(c6ccccc6-5)c5ccccc5-c5c4ccc4ccccc54)c4ccccc34)cc21 ; CC1(c2ccccc2)c2ccccc2Oc2ccc(-c3ccc4c(c3)C(C)(c3ccccc3)c3ccc5ccccc5c3-4)cc21]

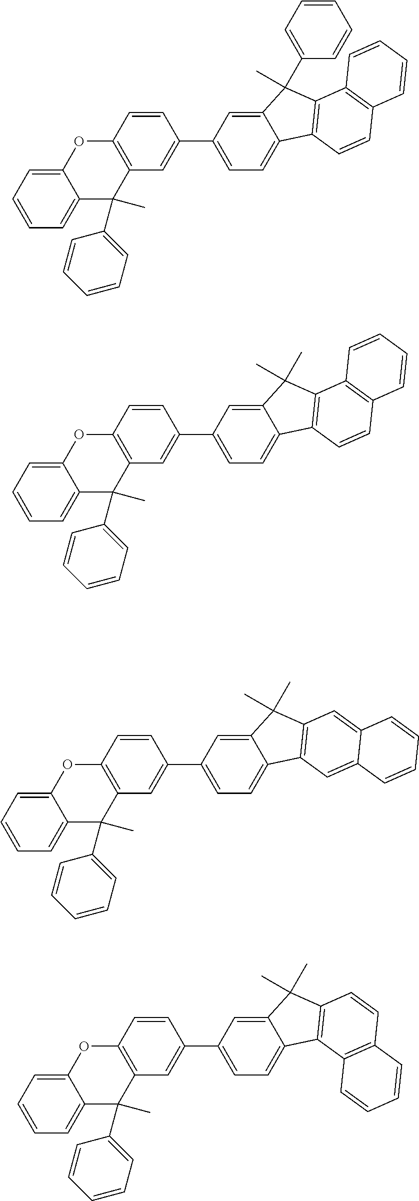 [SMILES: CC1(c2ccccc2)c2ccccc2Oc2ccc(-c3ccc4c(c3)C(C)(c3ccccc3)c3c-4ccc4ccccc34)cc21 ; CC1(C)c2cc(-c3ccc4c(c3)C(C)(c3ccccc3)c3ccccc3O4)ccc2-c2ccc3ccccc3c21 ; CC1(C)c2cc(-c3ccc4c(c3)C(C)(c3ccccc3)c3ccccc3O4)ccc2-c2cc3ccccc3cc21 ; CC1(C)c2cc(-c3ccc4c(c3)C(C)(c3ccccc3)c3ccccc3O4)ccc2-c2c1ccc1ccccc21]

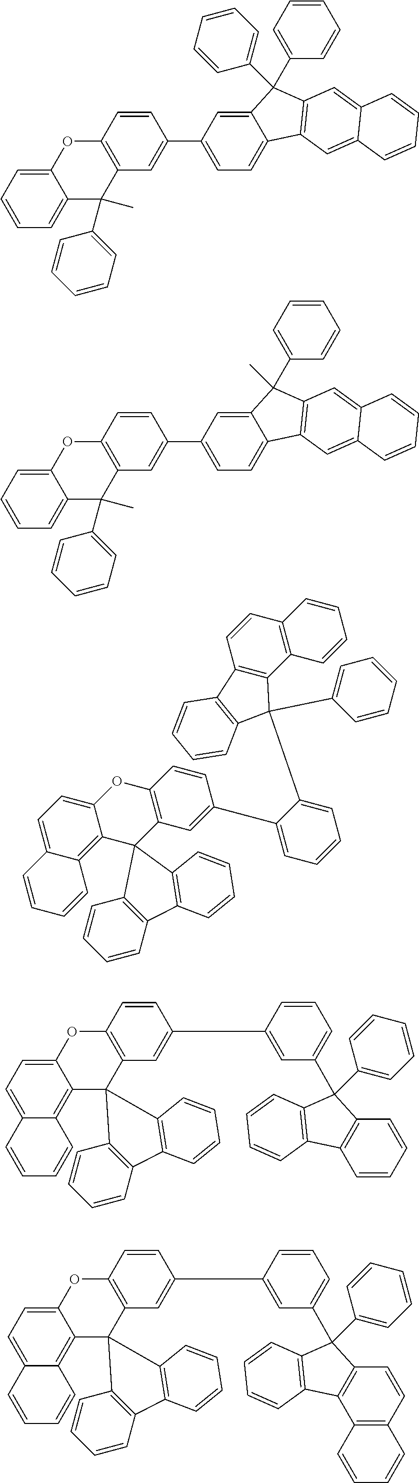

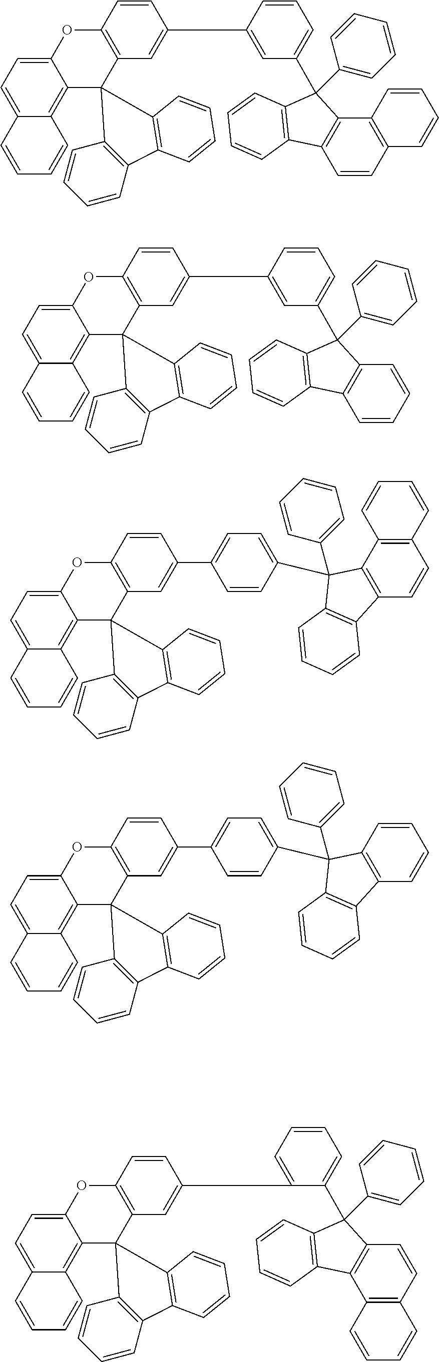 [SMILES: c1ccc(C2(c3cccc(-c4ccc5c(c4)C4(c6ccccc6-c6ccccc64)c4c(ccc6ccccc46)O5)c3)c3ccccc3-c3ccc4ccccc4c32)cc1 ; c1ccc(C2(c3cccc(-c4ccc5c(c4)C4(c6ccccc6-c6ccccc64)c4c(ccc6ccccc46)O5)c3)c3ccccc3-c3ccccc32)cc1 ; c1ccc(C2(c3ccc(-c4ccc5c(c4)C4(c6ccccc6-c6ccccc64)c4c(ccc6ccccc46)O5)cc3)c3ccccc3-c3ccc4ccccc4c32)cc1 ; c1ccc(C2(c3ccc(-c4ccc5c(c4)C4(c6ccccc6-c6ccccc64)c4c(ccc6ccccc46)O5)cc3)c3ccccc3-c3ccccc32)cc1 ; c1ccc(C2(c3ccccc3-c3ccc4c(c3)C3(c5ccccc5-c5ccccc53)c3c(ccc5ccccc35)O4)c3ccccc3-c3c2ccc2ccccc32)cc1]

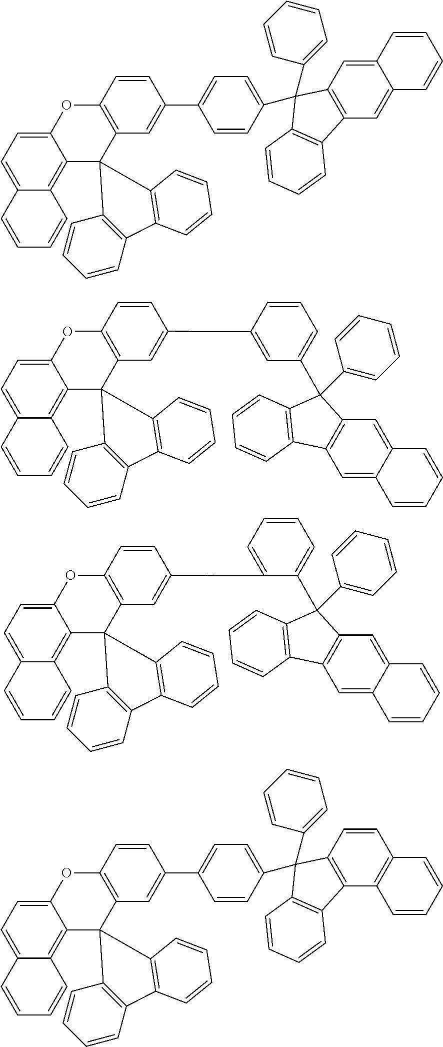

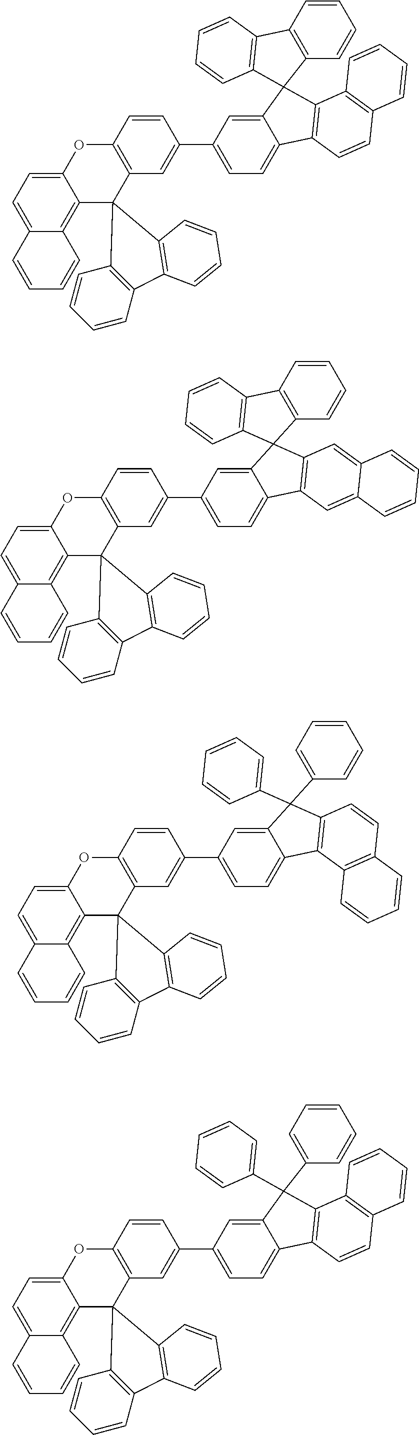 [SMILES: c1ccc2c(c1)-c1ccccc1C21c2cc(-c3ccc4c(c3)C3(c5ccccc5-c5ccccc53)c3c-4ccc4ccccc34)ccc2Oc2ccc3ccccc3c21 ; c1ccc2c(c1)-c1ccccc1C21c2cc(-c3ccc4c(c3)C3(c5ccccc5-c5ccccc53)c3c(ccc5ccccc35)O4)ccc2-c2cc3ccccc3cc21 ; c1ccc(C2(c3ccccc3)c3cc(-c4ccc5c(c4)C4(c6ccccc6-c6ccccc64)c4c(ccc6ccccc46)O5)ccc3-c3c2ccc2ccccc32)cc1 ; c1ccc(C2(c3ccccc3)c3cc(-c4ccc5c(c4)C4(c6ccccc6-c6ccccc64)c4c(ccc6ccccc46)O5)ccc3-c3ccc4ccccc4c32)cc1]

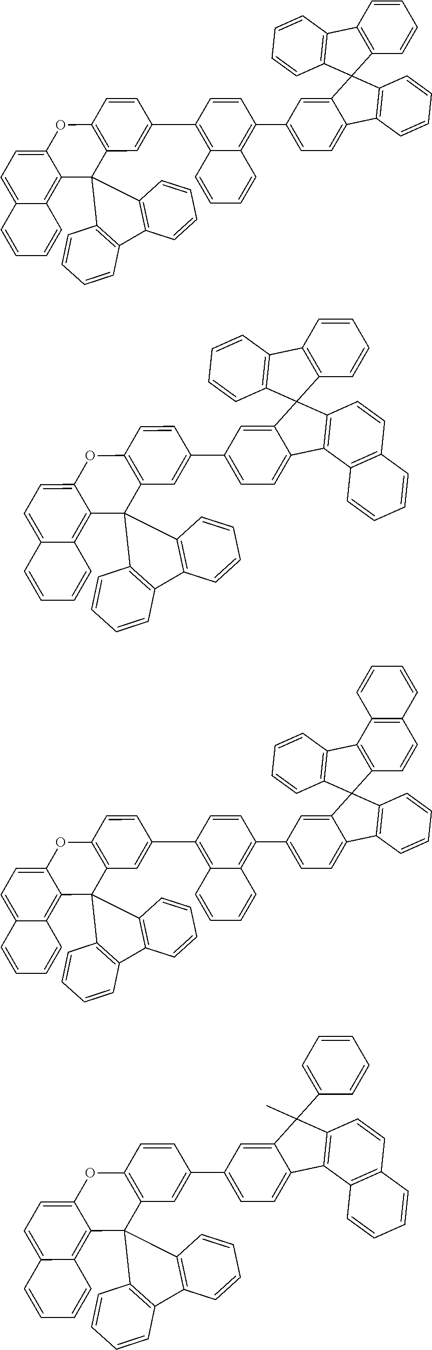 [SMILES: c1ccc2c(c1)-c1ccccc1C21c2ccccc2-c2ccc(-c3ccc(-c4ccc5c(c4)C4(c6ccccc6-c6ccccc64)c4c(ccc6ccccc46)O5)c4ccccc34)cc21 ; c1ccc2c(c1)-c1ccccc1C21c2cc(-c3ccc4c(c3)C3(c5ccccc5-c5ccccc53)c3c(ccc5ccccc35)O4)ccc2-c2c1ccc1ccccc21 ; c1ccc2c(c1)-c1ccc(-c3ccc(-c4ccc5c(c4)C4(c6ccccc6-c6ccccc64)c4c(ccc6ccccc46)O5)c4ccccc34)cc1C21c2ccccc2-c2c1ccc1ccccc21 ; CC1(c2ccccc2)c2cc(-c3ccc4c(c3)C3(c5ccccc5-c5ccccc53)c3c(ccc5ccccc35)O4)ccc2-c2c1ccc1ccccc21]

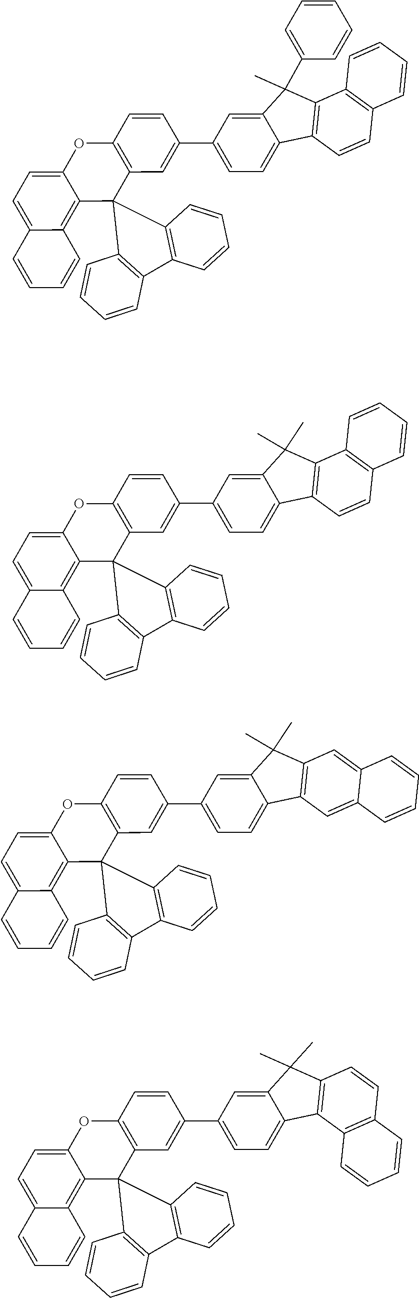 [SMILES: CC1(c2ccccc2)c2cc(-c3ccc4c(c3)C3(c5ccccc5-c5ccccc53)c3c(ccc5ccccc35)O4)ccc2-c2ccc3ccccc3c21 ; CC1(C)c2cc(-c3ccc4c(c3)C3(c5ccccc5-c5ccccc53)c3c(ccc5ccccc35)O4)ccc2-c2ccc3ccccc3c21 ; CC1(C)c2cc(-c3ccc4c(c3)C3(c5ccccc5-c5ccccc53)c3c(ccc5ccccc35)O4)ccc2-c2cc3ccccc3cc21 ; CC1(C)c2cc(-c3ccc4c(c3)C3(c5ccccc5-c5ccccc53)c3c(ccc5ccccc35)O4)ccc2-c2c1ccc1ccccc21]

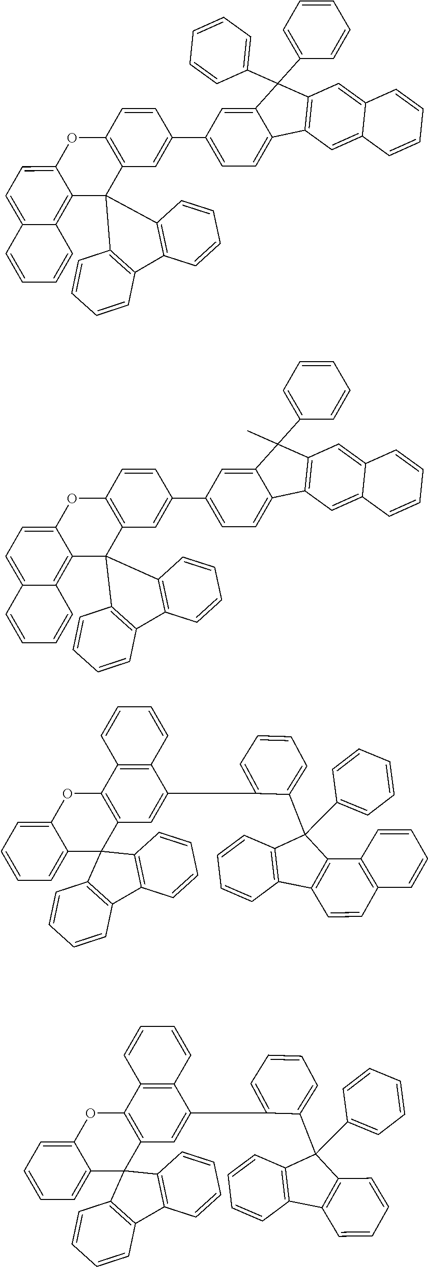

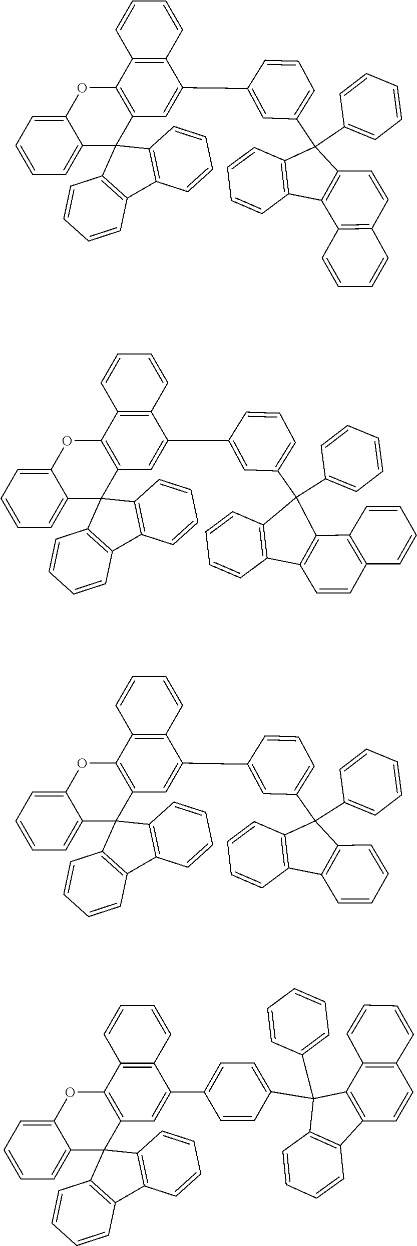 [SMILES: c1ccc(C2(c3cccc(-c4cc5c(c6ccccc46)Oc4ccccc4C54c5ccccc5-c5ccccc54)c3)c3ccccc3-c3c2ccc2ccccc32)cc1 ; c1ccc(C2(c3cccc(-c4cc5c(c6ccccc46)Oc4ccccc4C54c5ccccc5-c5ccccc54)c3)c3ccccc3-c3ccc4ccccc4c32)cc1 ; c1ccc(C2(c3cccc(-c4cc5c(c6ccccc46)Oc4ccccc4C54c5ccccc5-c5ccccc54)c3)c3ccccc3-c3ccccc32)cc1 ; c1ccc(C2(c3ccc(-c4cc5c(c6ccccc46)Oc4ccccc4C54c5ccccc5-c5ccccc54)cc3)c3ccccc3-c3ccc4ccccc4c32)cc1]

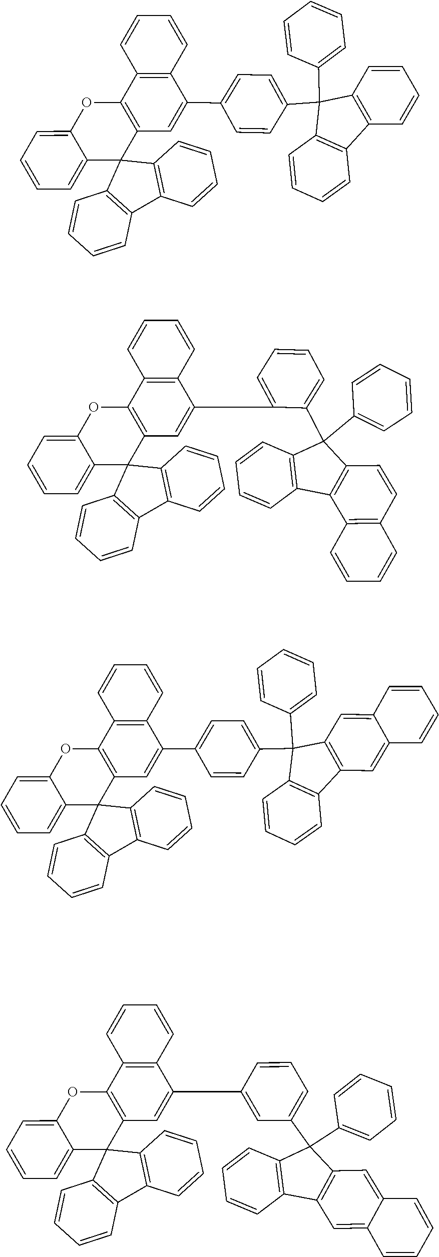 [SMILES: c1ccc(C2(c3ccc(-c4cc5c(c6ccccc46)Oc4ccccc4C54c5ccccc5-c5ccccc54)cc3)c3ccccc3-c3ccccc32)cc1 ; c1ccc(C2(c3ccccc3-c3cc4c(c5ccccc35)Oc3ccccc3C43c4ccccc4-c4ccccc43)c3ccccc3-c3c2ccc2ccccc32)cc1 ; c1ccc(C2(c3ccc(-c4cc5c(c6ccccc46)Oc4ccccc4C54c5ccccc5-c5ccccc54)cc3)c3ccccc3-c3cc4ccccc4cc32)cc1 ; c1ccc(C2(c3cccc(-c4cc5c(c6ccccc46)Oc4ccccc4C54c5ccccc5-c5ccccc54)c3)c3ccccc3-c3cc4ccccc4cc32)cc1]

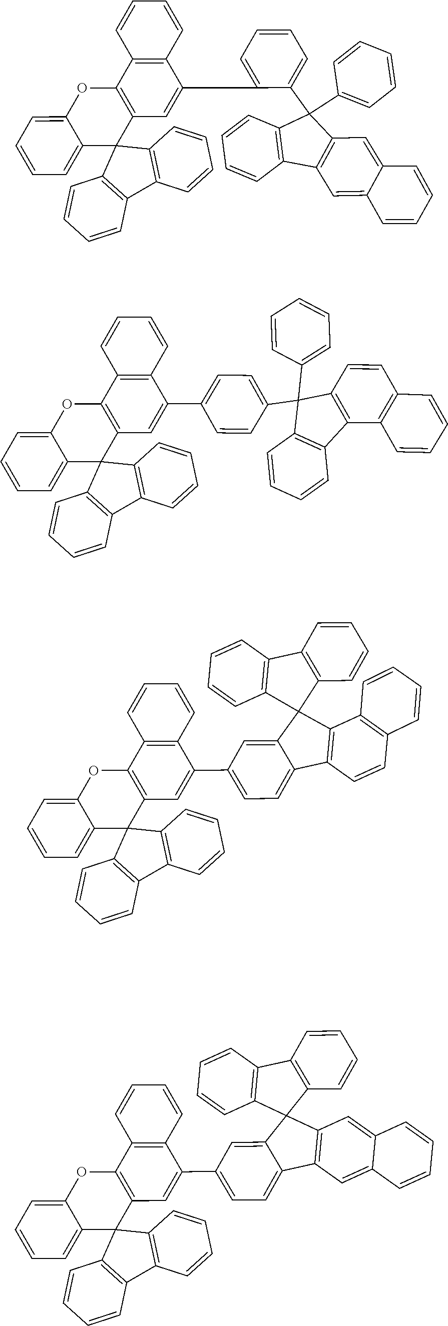 [SMILES: c1ccc(C2(c3ccccc3-c3cc4c(c5ccccc35)Oc3ccccc3C43c4ccccc4-c4ccccc43)c3ccccc3-c3cc4ccccc4cc32)cc1 ; c1ccc(C2(c3ccc(-c4cc5c(c6ccccc46)Oc4ccccc4C54c5ccccc5-c5ccccc54)cc3)c3ccccc3-c3c2ccc2ccccc32)cc1 ; c1ccc2c(c1)Oc1c(cc(-c3ccc4c(c3)C3(c5ccccc5-c5ccccc53)c3c-4ccc4ccccc34)c3ccccc13)C21c2ccccc2-c2ccccc21 ; c1ccc2c(c1)Oc1c(cc(-c3ccc4c(c3)C3(c5ccccc5-c5ccccc53)c3cc5ccccc5cc3-4)c3ccccc13)C21c2ccccc2-c2ccccc21]

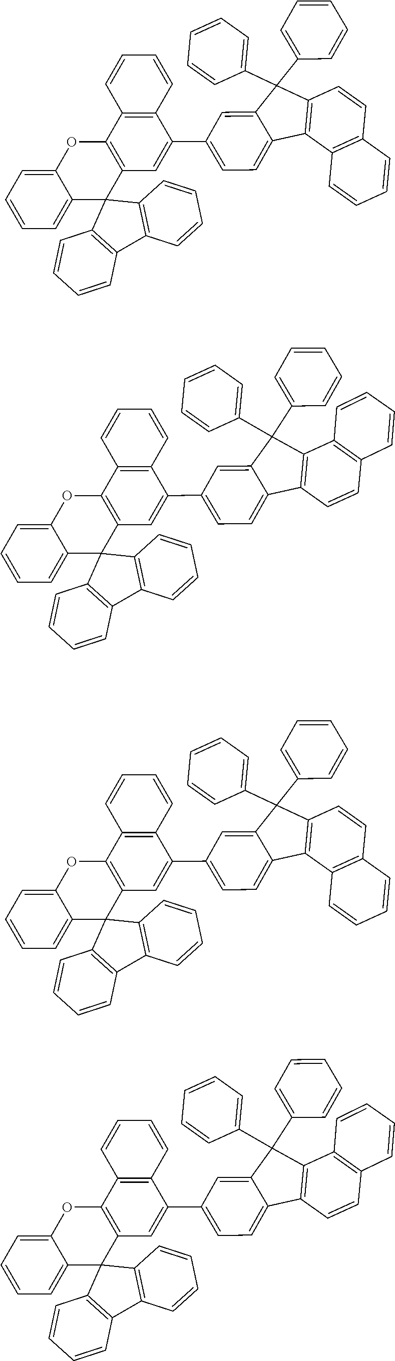 [SMILES: c1ccc(C2(c3ccccc3)c3cc(-c4cc5c(c6ccccc46)Oc4ccccc4C54c5ccccc5-c5ccccc54)ccc3-c3c2ccc2ccccc32)cc1 ; c1ccc(C2(c3ccccc3)c3cc(-c4cc5c(c6ccccc46)Oc4ccccc4C54c5ccccc5-c5ccccc54)ccc3-c3ccc4ccccc4c32)cc1 ; c1ccc(C2(c3ccccc3)c3cc(-c4cc5c(c6ccccc46)Oc4ccccc4C54c5ccccc5-c5ccccc54)ccc3-c3c2ccc2ccccc32)cc1 ; c1ccc(C2(c3ccccc3)c3cc(-c4cc5c(c6ccccc46)Oc4ccccc4C54c5ccccc5-c5ccccc54)ccc3-c3ccc4ccccc4c32)cc1]

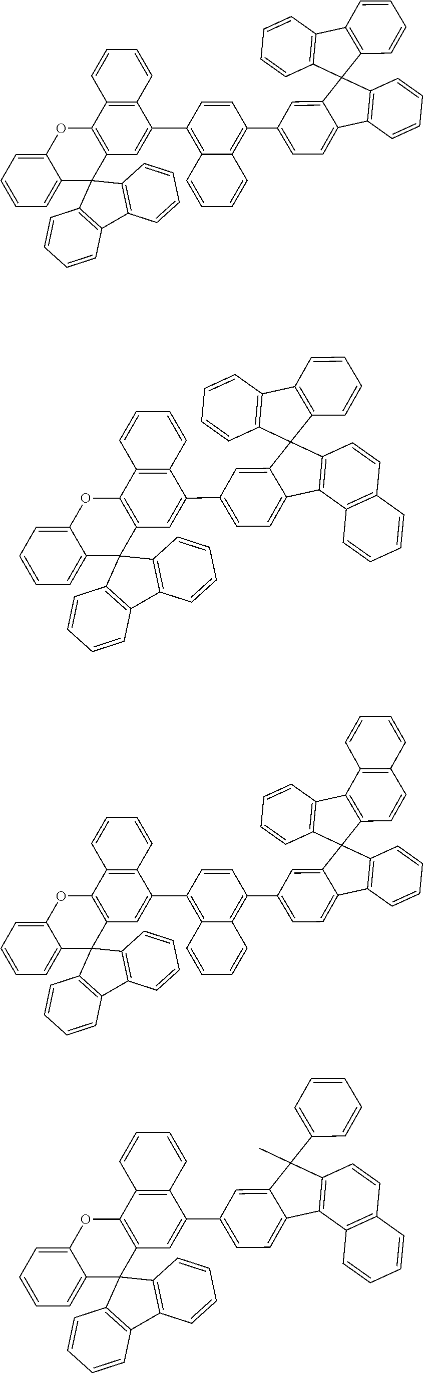 [SMILES: c1ccc2c(c1)Oc1c(cc(-c3ccc(-c4ccc5c(c4)C4(c6ccccc6-c6ccccc64)c4ccccc4-5)c4ccccc34)c3ccccc13)C21c2ccccc2-c2ccccc21 ; c1ccc2c(c1)Oc1c(cc(-c3ccc4c(c3)C3(c5ccccc5-c5ccccc53)c3ccc5ccccc5c3-4)c3ccccc13)C21c2ccccc2-c2ccccc21 ; c1ccc2c(c1)Oc1c(cc(-c3ccc(-c4ccc5c(c4)C4(c6ccccc6-5)c5ccccc5-c5c4ccc4ccccc54)c4ccccc34)c3ccccc13)C21c2ccccc2-c2ccccc21 ; CC1(c2ccccc2)c2cc(-c3cc4c(c5ccccc35)Oc3ccccc3C43c4ccccc4-c4ccccc43)ccc2-c2c1ccc1ccccc21]

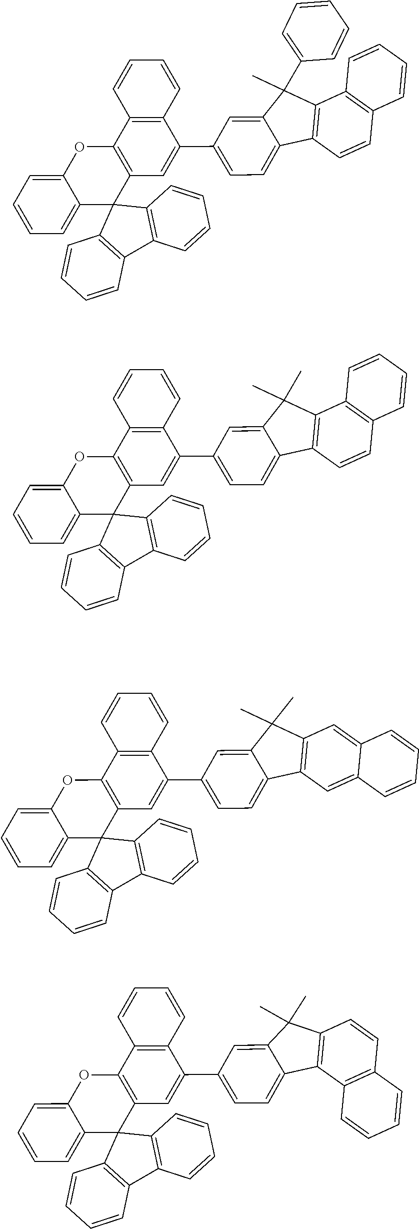 [SMILES: CC1(c2ccccc2)c2cc(-c3cc4c(c5ccccc35)Oc3ccccc3C43c4ccccc4-c4ccccc43)ccc2-c2ccc3ccccc3c21 ; CC1(C)c2cc(-c3cc4c(c5ccccc35)Oc3ccccc3C43c4ccccc4-c4ccccc43)ccc2-c2ccc3ccccc3c21 ; CC1(C)c2cc(-c3cc4c(c5ccccc35)Oc3ccccc3C43c4ccccc4-c4ccccc43)ccc2-c2cc3ccccc3cc21 ; CC1(C)c2cc(-c3cc4c(c5ccccc35)Oc3ccccc3C43c4ccccc4-c4ccccc43)ccc2-c2c1ccc1ccccc21]

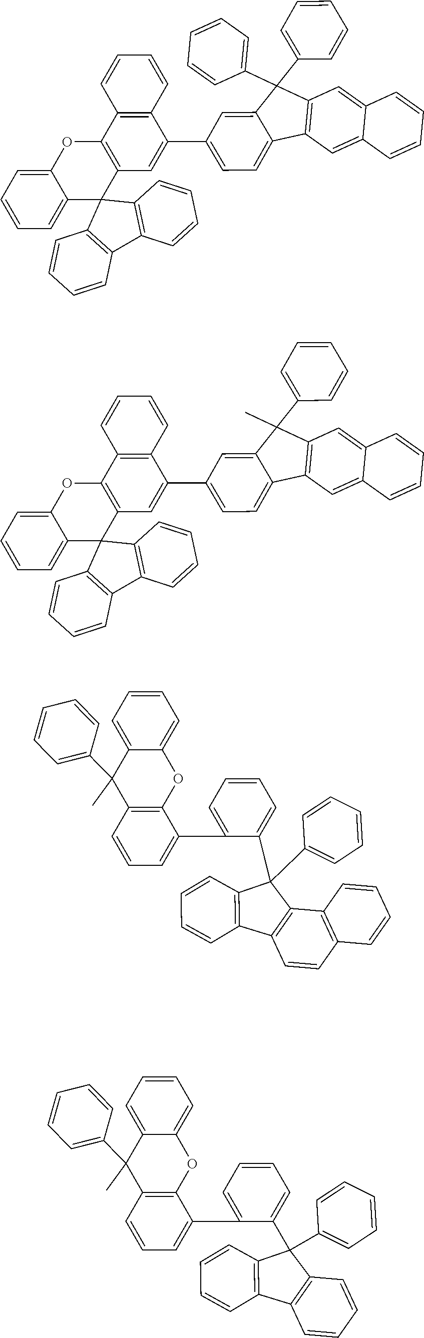 [SMILES: c1ccc(C2(c3ccccc3)c3cc(-c4cc5c(c6ccccc46)Oc4ccccc4C54c5ccccc5-c5ccccc54)ccc3-c3cc4ccccc4cc32)cc1 ; CC1(c2ccccc2)c2cc(-c3cc4c(c5ccccc35)Oc3ccccc3C43c4ccccc4-c4ccccc43)ccc2-c2cc3ccccc3cc21 ; CC1(c2ccccc2)c2ccccc2Oc2c(-c3ccccc3C3(c4ccccc4)c4ccccc4-c4ccc5ccccc5c43)cccc21 ; CC1(c2ccccc2)c2ccccc2Oc2c(-c3ccccc3C3(c4ccccc4)c4ccccc4-c4ccccc43)cccc21]

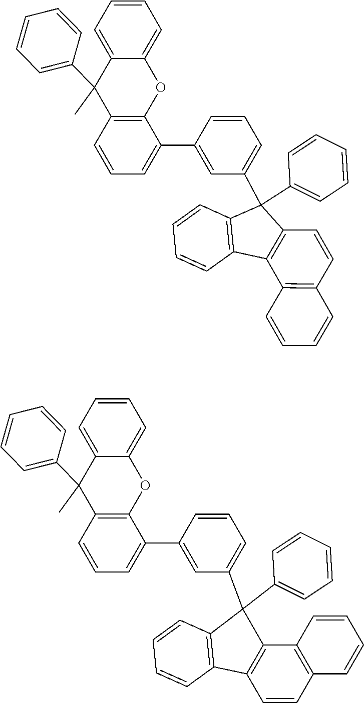 [SMILES: CC1(c2ccccc2)c2ccccc2Oc2c(-c3cccc(C4(c5ccccc5)c5ccccc5-c5c4ccc4ccccc54)c3)cccc21 ; CC1(c2ccccc2)c2ccccc2Oc2c(-c3cccc(C4(c5ccccc5)c5ccccc5-c5ccc6ccccc6c54)c3)cccc21]

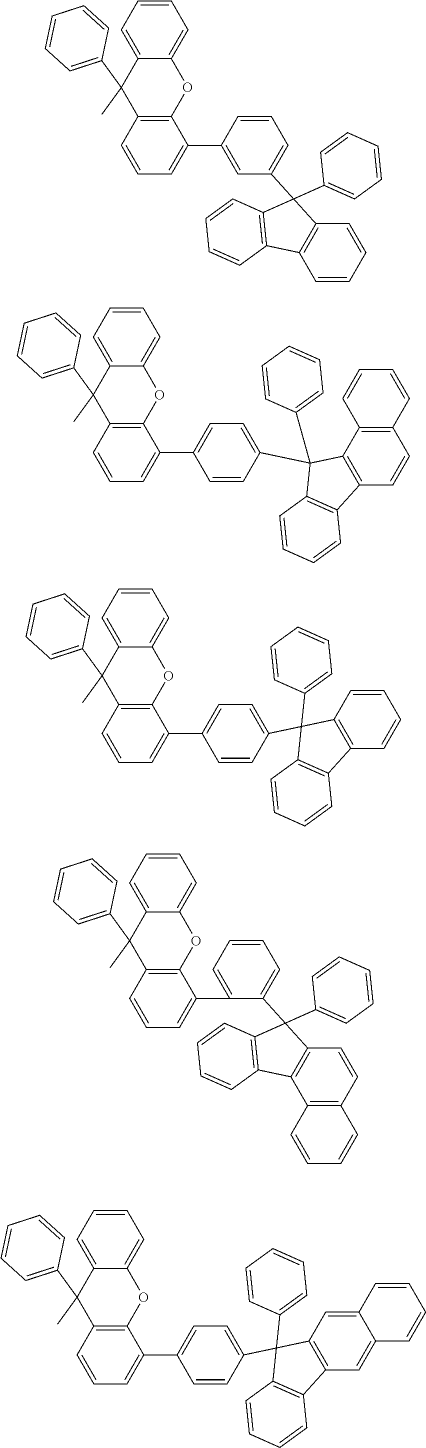 [SMILES: CC1(c2ccccc2)c2ccccc2Oc2c(-c3cccc(C4(c5ccccc5)c5ccccc5-c5ccccc54)c3)cccc21 ; CC1(c2ccccc2)c2ccccc2Oc2c(-c3ccc(C4(c5ccccc5)c5ccccc5-c5ccc6ccccc6c54)cc3)cccc21 ; CC1(c2ccccc2)c2ccccc2Oc2c(-c3ccc(C4(c5ccccc5)c5ccccc5-c5ccccc54)cc3)cccc21 ; CC1(c2ccccc2)c2ccccc2Oc2c(-c3ccccc3C3(c4ccccc4)c4ccccc4-c4c3ccc3ccccc43)cccc21 ; CC1(c2ccccc2)c2ccccc2Oc2c(-c3ccc(C4(c5ccccc5)c5ccccc5-c5cc6ccccc6cc54)cc3)cccc21]

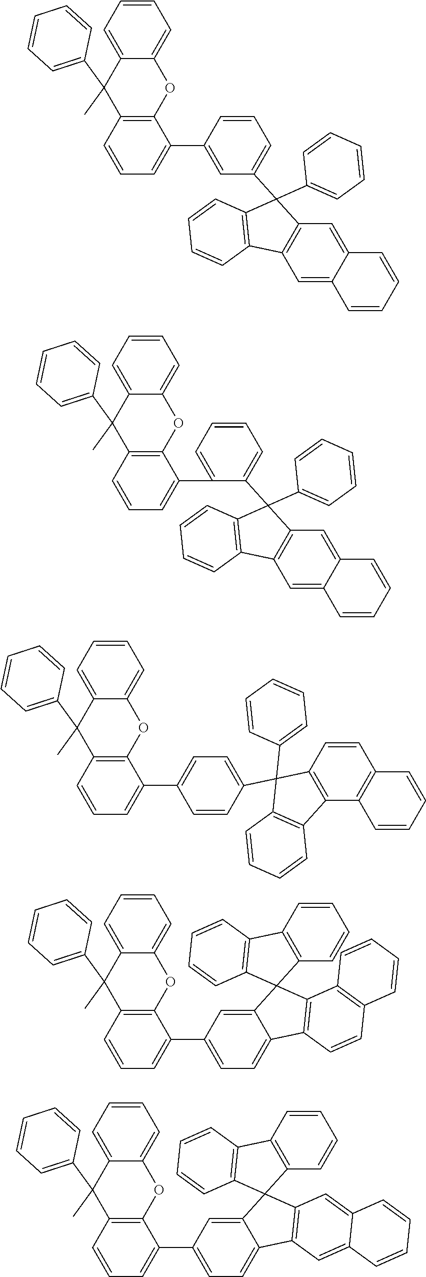

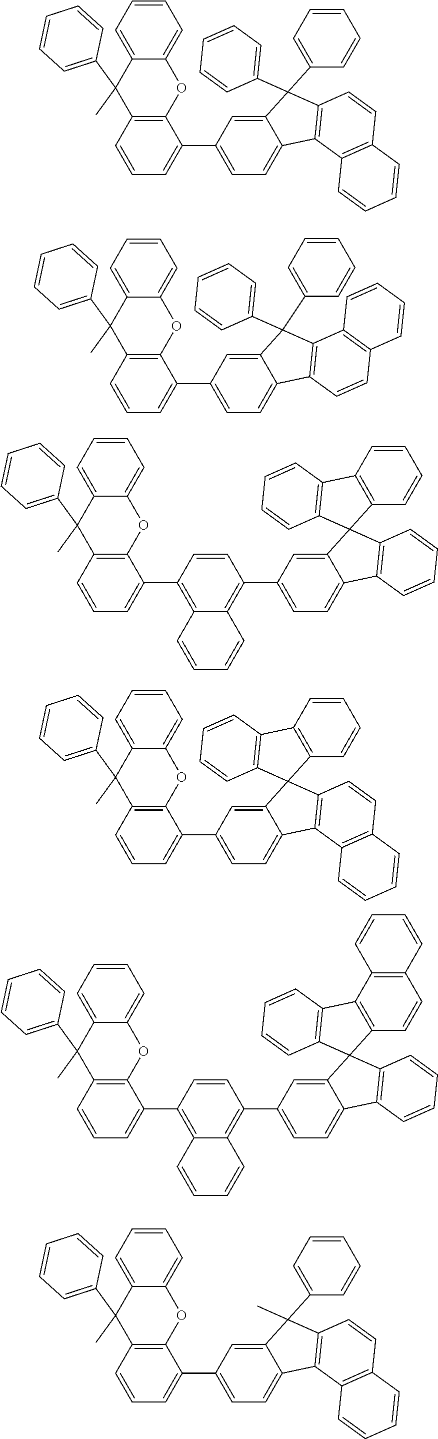

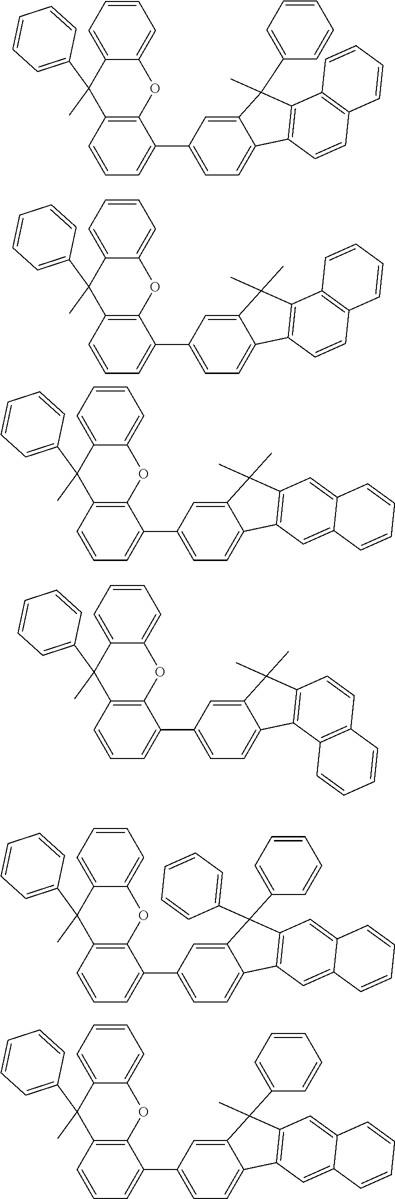

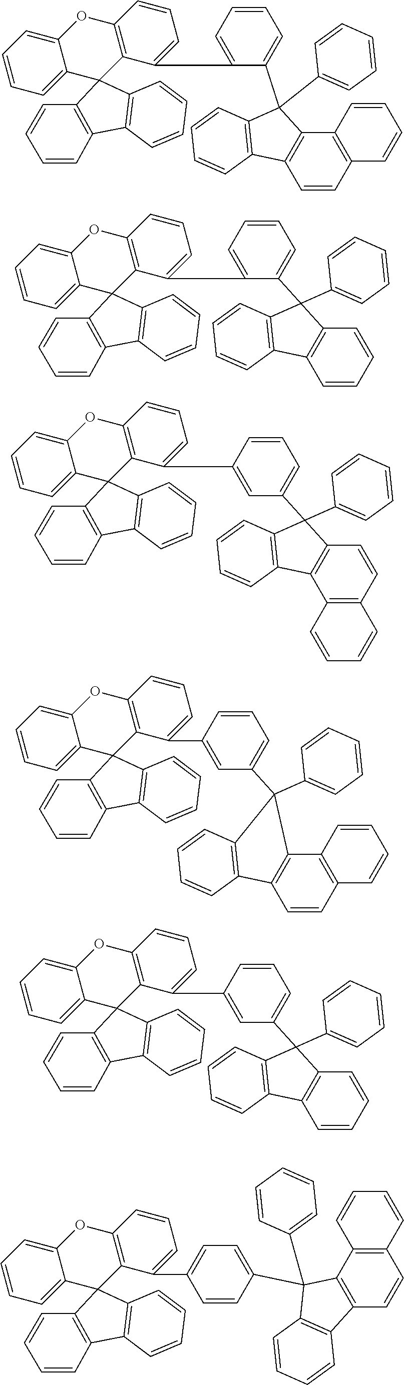

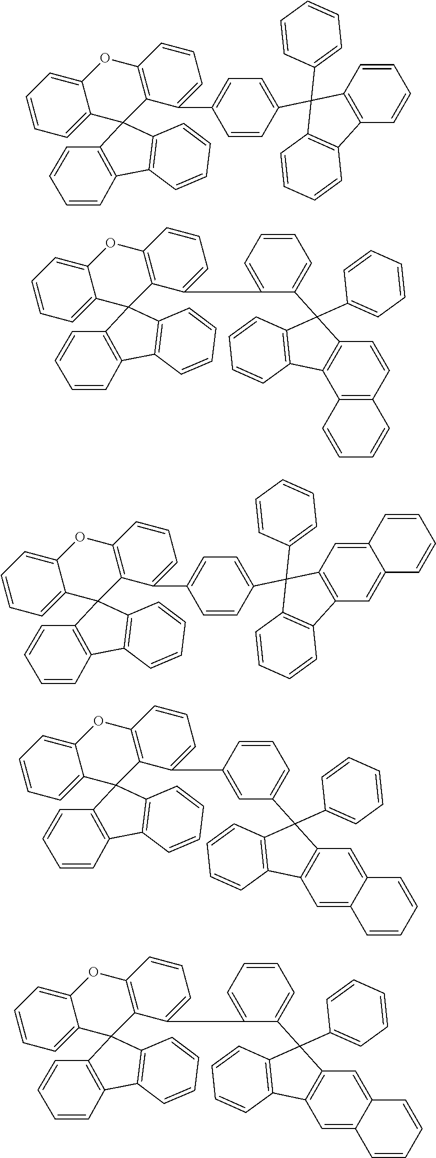

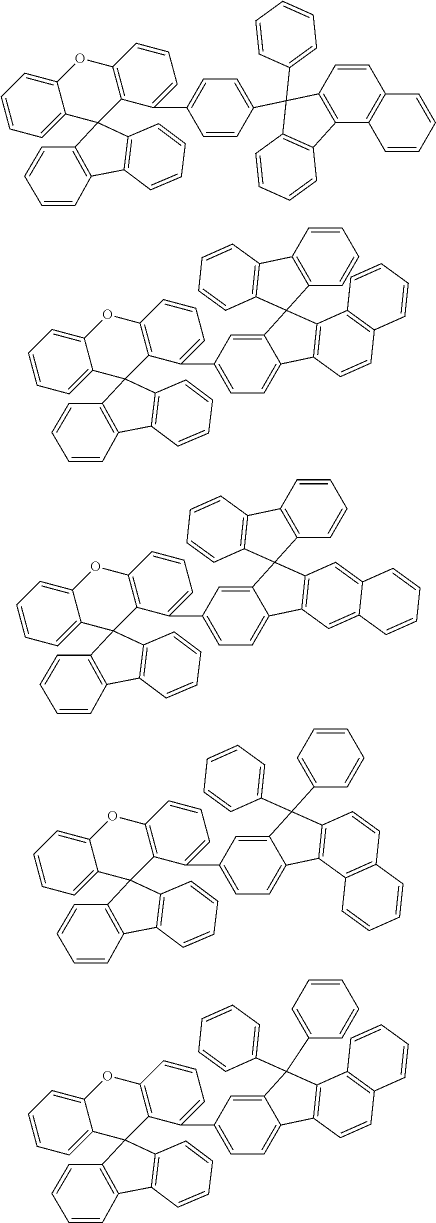

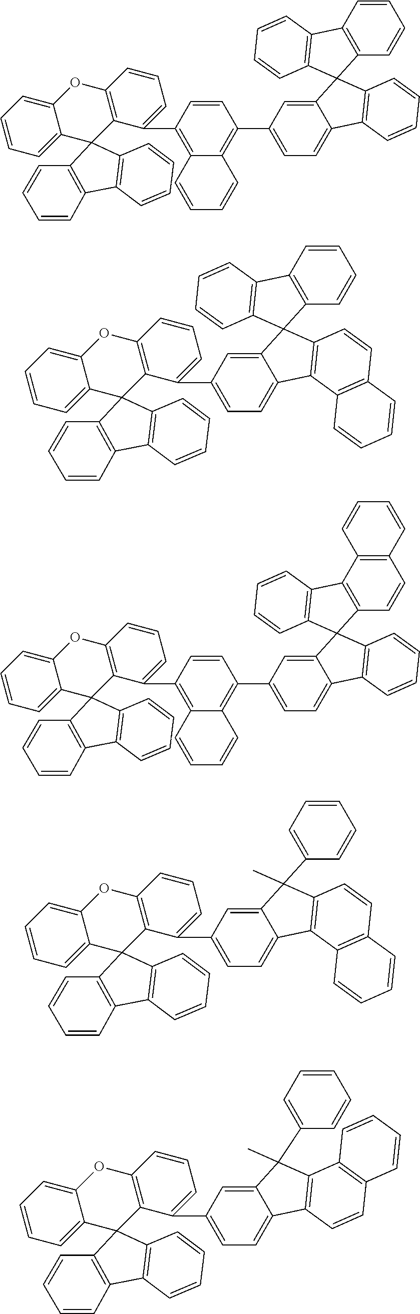 [SMILES: c1ccc2c(c1)Oc1cccc(-c3ccc(-c4ccc5c(c4)C4(c6ccccc6-c6ccccc64)c4ccccc4-5)c4ccccc34)c1C21c2ccccc2-c2ccccc21 ; c1ccc2c(c1)Oc1cccc(-c3ccc4c(c3)C3(c5ccccc5-c5ccccc53)c3ccc5ccccc5c3-4)c1C21c2ccccc2-c2ccccc21 ; c1ccc2c(c1)Oc1cccc(-c3ccc(-c4ccc5c(c4)C4(c6ccccc6-5)c5ccccc5-c5c4ccc4ccccc54)c4ccccc34)c1C21c2ccccc2-c2ccccc21 ; CC1(c2ccccc2)c2cc(-c3cccc4c3C3(c5ccccc5O4)c4ccccc4-c4ccccc43)ccc2-c2c1ccc1ccccc21 ; CC1(c2ccccc2)c2cc(-c3cccc4c3C3(c5ccccc5O4)c4ccccc4-c4ccccc43)ccc2-c2ccc3ccccc3c21]

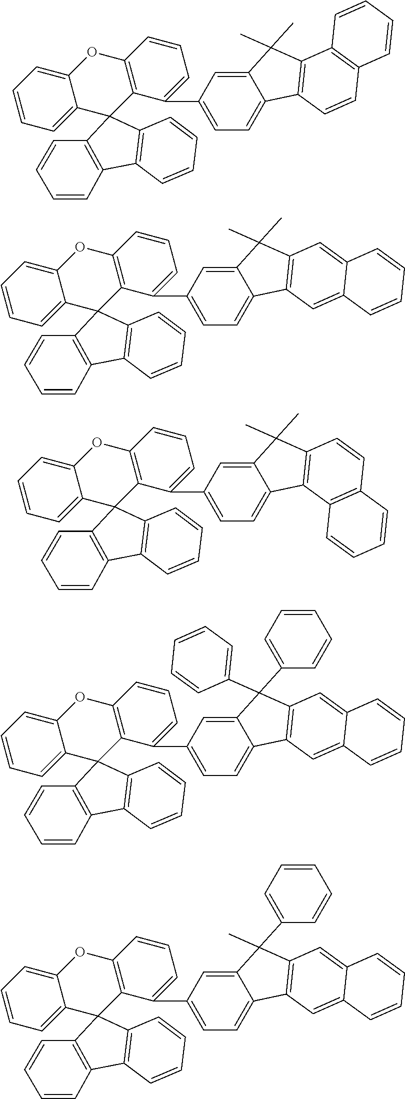 [SMILES: CC1(C)c2cc(-c3cccc4c3C3(c5ccccc5O4)c4ccccc4-c4ccccc43)ccc2-c2ccc3ccccc3c21 ; CC1(C)c2cc(-c3cccc4c3C3(c5ccccc5O4)c4ccccc4-c4ccccc43)ccc2-c2cc3ccccc3cc21 ; CC1(C)c2cc(-c3cccc4c3C3(c5ccccc5O4)c4ccccc4-c4ccccc43)ccc2-c2c1ccc1ccccc21 ; c1ccc(C2(c3ccccc3)c3cc(-c4cccc5c4C4(c6ccccc6O5)c5ccccc5-c5ccccc54)ccc3-c3cc4ccccc4cc32)cc1 ; CC1(c2ccccc2)c2cc(-c3cccc4c3C3(c5ccccc5O4)c4ccccc4-c4ccccc43)ccc2-c2cc3ccccc3cc21]

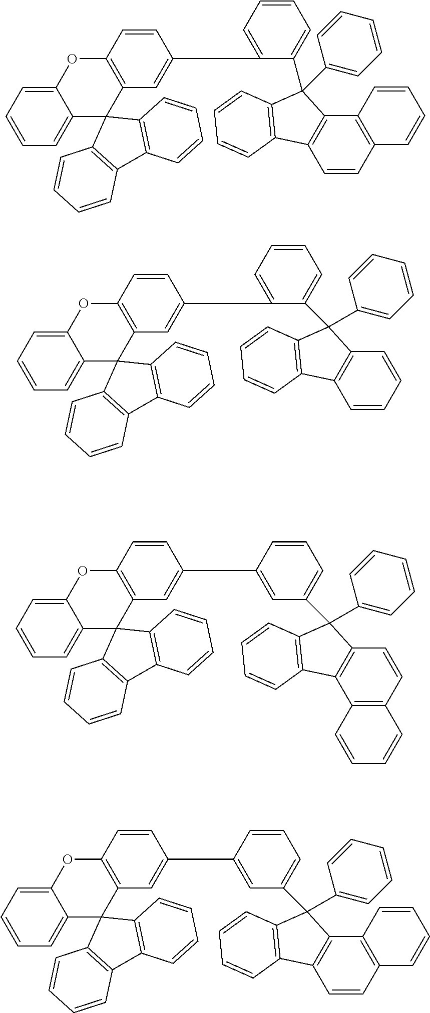 [SMILES: c1ccc(C2(c3ccccc3-c3ccc4c(c3)C3(c5ccccc5O4)c4ccccc4-c4ccccc43)c3ccccc3-c3ccc4ccccc4c32)cc1 ; c1ccc(C2(c3ccccc3-c3ccc4c(c3)C3(c5ccccc5O4)c4ccccc4-c4ccccc43)c3ccccc3-c3ccccc32)cc1 ; c1ccc(C2(c3cccc(-c4ccc5c(c4)C4(c6ccccc6O5)c5ccccc5-c5ccccc54)c3)c3ccccc3-c3c2ccc2ccccc32)cc1 ; c1ccc(C2(c3cccc(-c4ccc5c(c4)C4(c6ccccc6O5)c5ccccc5-c5ccccc54)c3)c3ccccc3-c3ccc4ccccc4c32)cc1]

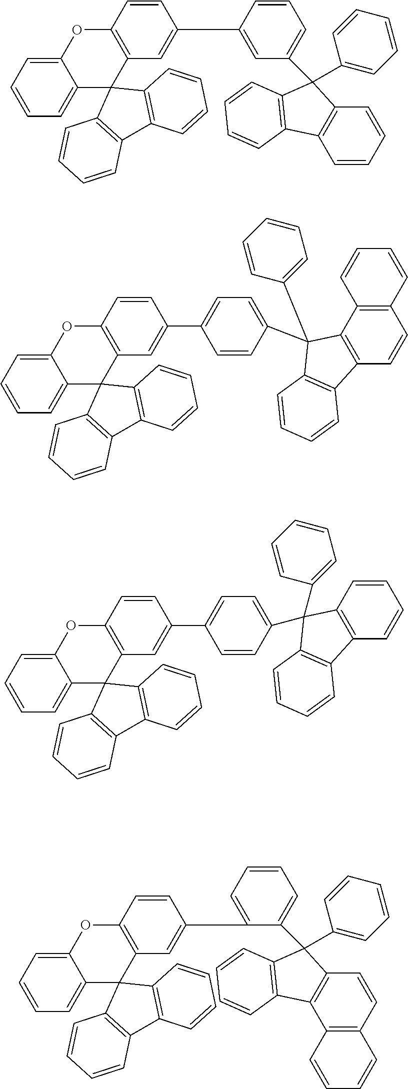 [SMILES: c1ccc(C2(c3cccc(-c4ccc5c(c4)C4(c6ccccc6O5)c5ccccc5-c5ccccc54)c3)c3ccccc3-c3ccccc32)cc1 ; c1ccc(C2(c3ccc(-c4ccc5c(c4)C4(c6ccccc6O5)c5ccccc5-c5ccccc54)cc3)c3ccccc3-c3ccc4ccccc4c32)cc1 ; c1ccc(C2(c3ccc(-c4ccc5c(c4)C4(c6ccccc6O5)c5ccccc5-c5ccccc54)cc3)c3ccccc3-c3ccccc32)cc1 ; c1ccc(C2(c3ccccc3-c3ccc4c(c3)C3(c5ccccc5O4)c4ccccc4-c4ccccc43)c3ccccc3-c3c2ccc2ccccc32)cc1]

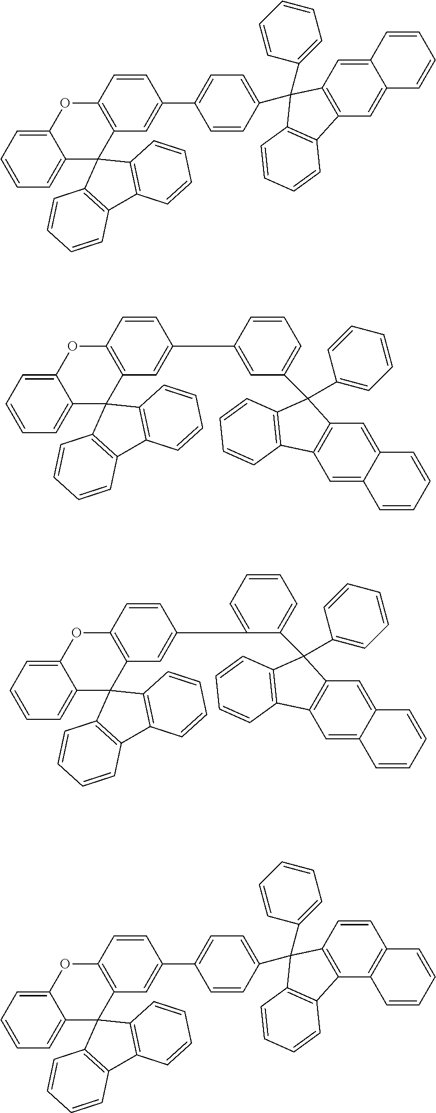 [SMILES: c1ccc(C2(c3ccc(-c4ccc5c(c4)C4(c6ccccc6O5)c5ccccc5-c5ccccc54)cc3)c3ccccc3-c3cc4ccccc4cc32)cc1 ; c1ccc(C2(c3cccc(-c4ccc5c(c4)C4(c6ccccc6O5)c5ccccc5-c5ccccc54)c3)c3ccccc3-c3cc4ccccc4cc32)cc1 ; c1ccc(C2(c3ccccc3-c3ccc4c(c3)C3(c5ccccc5O4)c4ccccc4-c4ccccc43)c3ccccc3-c3cc4ccccc4cc32)cc1 ; c1ccc(C2(c3ccc(-c4ccc5c(c4)C4(c6ccccc6O5)c5ccccc5-c5ccccc54)cc3)c3ccccc3-c3c2ccc2ccccc32)cc1]

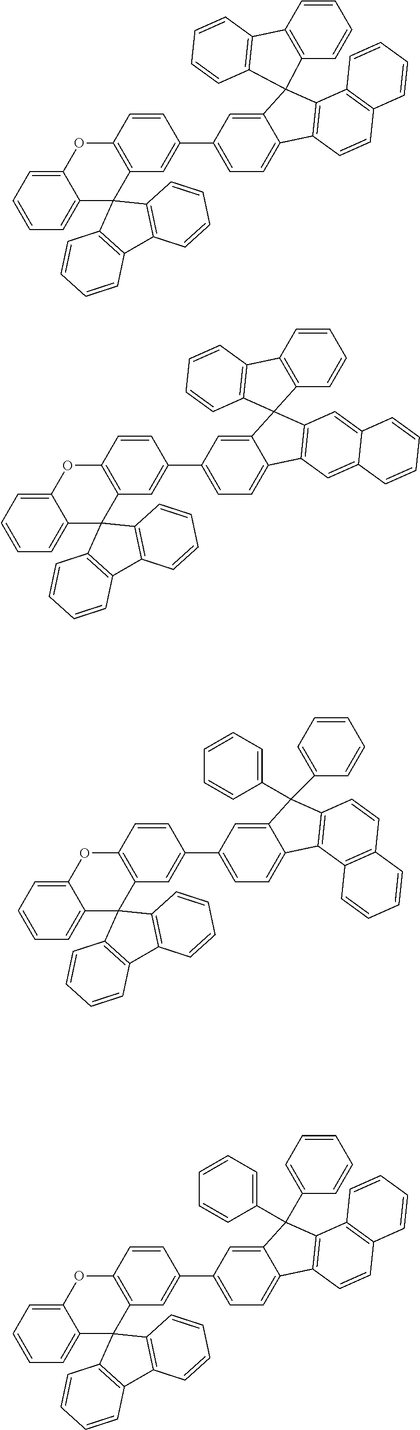 [SMILES: c1ccc2c(c1)Oc1ccc(-c3ccc4c(c3)C3(c5ccccc5-c5ccccc53)c3c-4ccc4ccccc34)cc1C21c2ccccc2-c2ccccc21 ; c1ccc2c(c1)Oc1ccc(-c3ccc4c(c3)C3(c5ccccc5-c5ccccc53)c3cc5ccccc5cc3-4)cc1C21c2ccccc2-c2ccccc21 ; c1ccc(C2(c3ccccc3)c3cc(-c4ccc5c(c4)C4(c6ccccc6O5)c5ccccc5-c5ccccc54)ccc3-c3c2ccc2ccccc32)cc1 ; c1ccc(C2(c3ccccc3)c3cc(-c4ccc5c(c4)C4(c6ccccc6O5)c5ccccc5-c5ccccc54)ccc3-c3ccc4ccccc4c32)cc1]

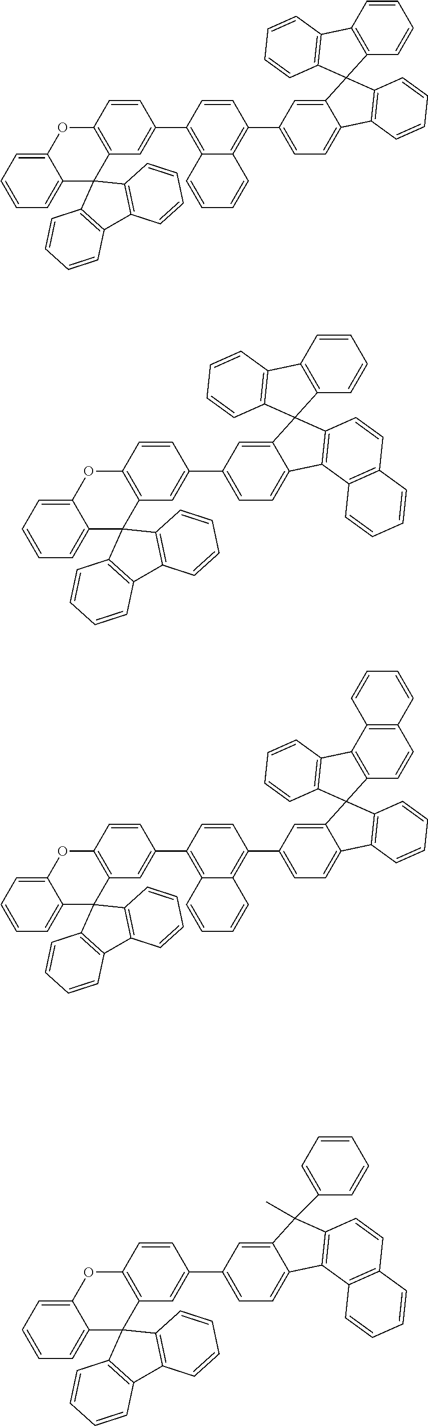 [SMILES: c1ccc2c(c1)Oc1ccc(-c3ccc(-c4ccc5c(c4)C4(c6ccccc6-c6ccccc64)c4ccccc4-5)c4ccccc34)cc1C21c2ccccc2-c2ccccc21 ; c1ccc2c(c1)Oc1ccc(-c3ccc4c(c3)C3(c5ccccc5-c5ccccc53)c3ccc5ccccc5c3-4)cc1C21c2ccccc2-c2ccccc21 ; c1ccc2c(c1)Oc1ccc(-c3ccc(-c4ccc5c(c4)C4(c6ccccc6-5)c5ccccc5-c5c4ccc4ccccc54)c4ccccc34)cc1C21c2ccccc2-c2ccccc21 ; CC1(c2ccccc2)c2cc(-c3ccc4c(c3)C3(c5ccccc5O4)c4ccccc4-c4ccccc43)ccc2-c2c1ccc1ccccc21]

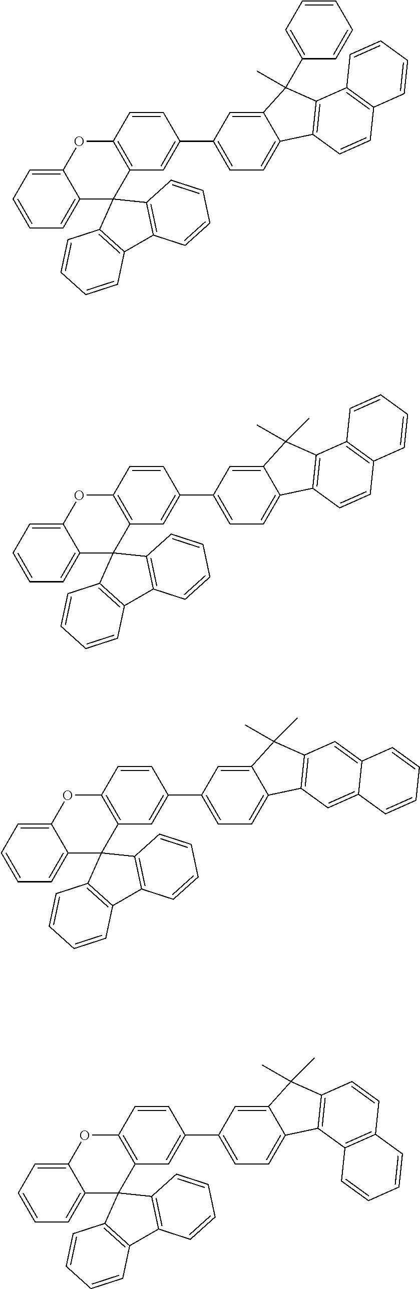 [SMILES: CC1(c2ccccc2)c2cc(-c3ccc4c(c3)C3(c5ccccc5O4)c4ccccc4-c4ccccc43)ccc2-c2ccc3ccccc3c21 ; CC1(C)c2cc(-c3ccc4c(c3)C3(c5ccccc5O4)c4ccccc4-c4ccccc43)ccc2-c2ccc3ccccc3c21 ; CC1(C)c2cc(-c3ccc4c(c3)C3(c5ccccc5O4)c4ccccc4-c4ccccc43)ccc2-c2cc3ccccc3cc21 ; CC1(C)c2cc(-c3ccc4c(c3)C3(c5ccccc5O4)c4ccccc4-c4ccccc43)ccc2-c2c1ccc1ccccc21]

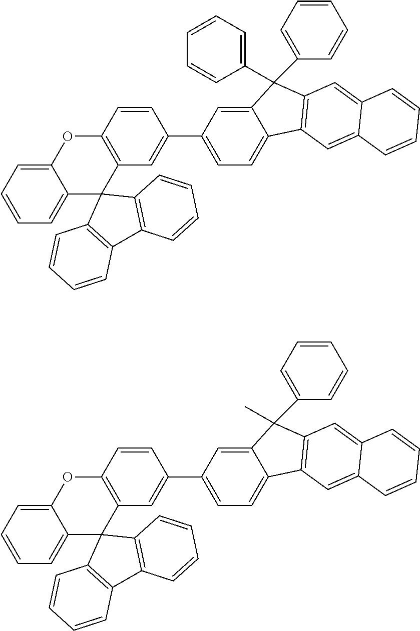 [SMILES: c1ccc(C2(c3ccccc3)c3cc(-c4ccc5c(c4)C4(c6ccccc6O5)c5ccccc5-c5ccccc54)ccc3-c3cc4ccccc4cc32)cc1 ; CC1(c2ccccc2)c2cc(-c3ccc4c(c3)C3(c5ccccc5O4)c4ccccc4-c4ccccc43)ccc2-c2cc3ccccc3cc21]

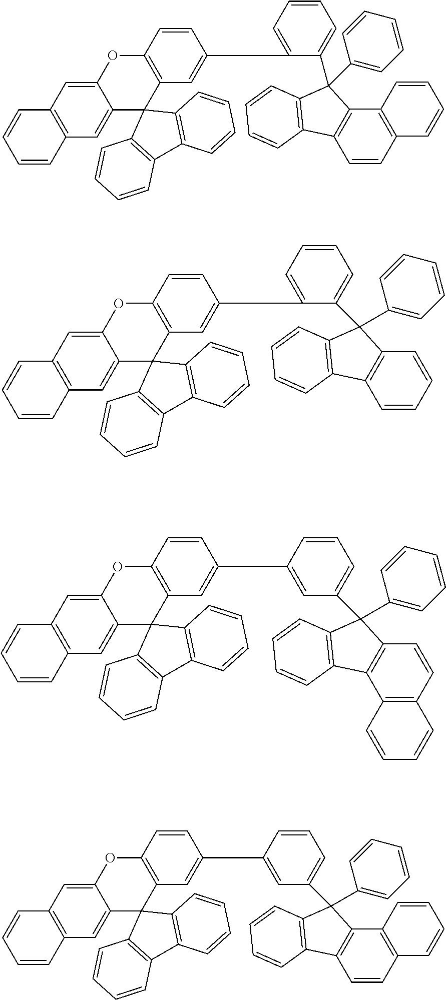 [SMILES: c1ccc(C2(c3ccccc3-c3ccc4c(c3)C3(c5cc6ccccc6cc5O4)c4ccccc4-c4ccccc43)c3ccccc3-c3ccc4ccccc4c32)cc1 ; c1ccc(C2(c3ccccc3-c3ccc4c(c3)C3(c5cc6ccccc6cc5O4)c4ccccc4-c4ccccc43)c3ccccc3-c3ccccc32)cc1 ; c1ccc(C2(c3cccc(-c4ccc5c(c4)C4(c6cc7ccccc7cc6O5)c5ccccc5-c5ccccc54)c3)c3ccccc3-c3c2ccc2ccccc32)cc1 ; c1ccc(C2(c3cccc(-c4ccc5c(c4)C4(c6cc7ccccc7cc6O5)c5ccccc5-c5ccccc54)c3)c3ccccc3-c3ccc4ccccc4c32)cc1]

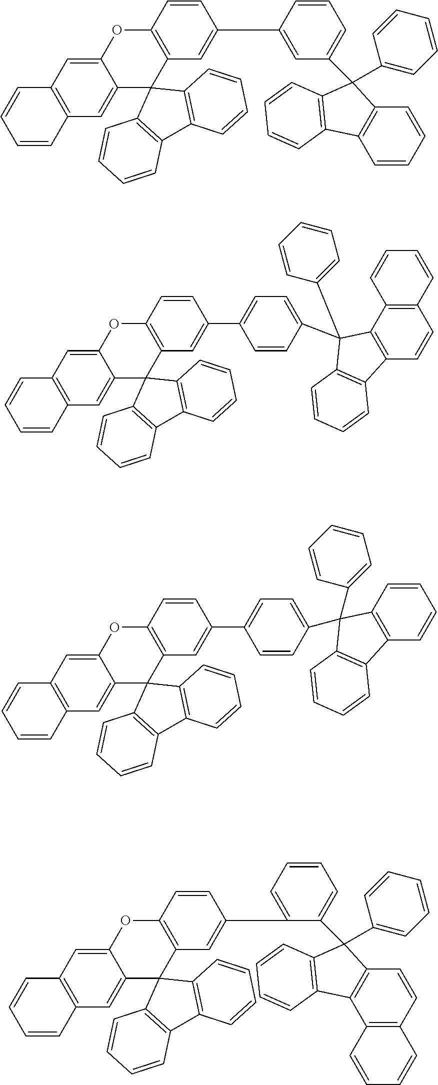 [SMILES: c1ccc(C2(c3cccc(-c4ccc5c(c4)C4(c6cc7ccccc7cc6O5)c5ccccc5-c5ccccc54)c3)c3ccccc3-c3ccccc32)cc1 ; c1ccc(C2(c3ccc(-c4ccc5c(c4)C4(c6cc7ccccc7cc6O5)c5ccccc5-c5ccccc54)cc3)c3ccccc3-c3ccc4ccccc4c32)cc1 ; c1ccc(C2(c3ccc(-c4ccc5c(c4)C4(c6cc7ccccc7cc6O5)c5ccccc5-c5ccccc54)cc3)c3ccccc3-c3ccccc32)cc1 ; c1ccc(C2(c3ccccc3-c3ccc4c(c3)C3(c5cc6ccccc6cc5O4)c4ccccc4-c4ccccc43)c3ccccc3-c3c2ccc2ccccc32)cc1]

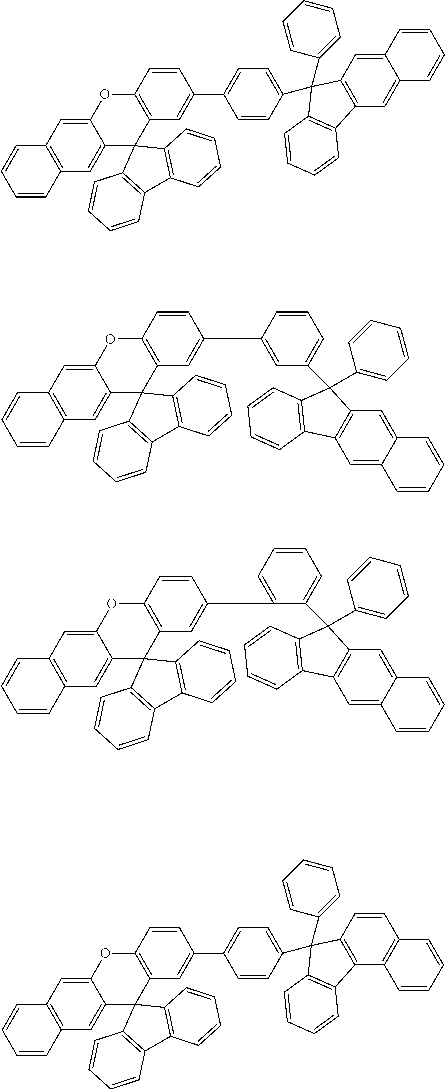 [SMILES: c1ccc(C2(c3ccc(-c4ccc5c(c4)C4(c6cc7ccccc7cc6O5)c5ccccc5-c5ccccc54)cc3)c3ccccc3-c3cc4ccccc4cc32)cc1 ; c1ccc(C2(c3cccc(-c4ccc5c(c4)C4(c6cc7ccccc7cc6O5)c5ccccc5-c5ccccc54)c3)c3ccccc3-c3cc4ccccc4cc32)cc1 ; c1ccc(C2(c3ccccc3-c3ccc4c(c3)C3(c5cc6ccccc6cc5O4)c4ccccc4-c4ccccc43)c3ccccc3-c3cc4ccccc4cc32)cc1 ; c1ccc(C2(c3ccc(-c4ccc5c(c4)C4(c6cc7ccccc7cc6O5)c5ccccc5-c5ccccc54)cc3)c3ccccc3-c3c2ccc2ccccc32)cc1]

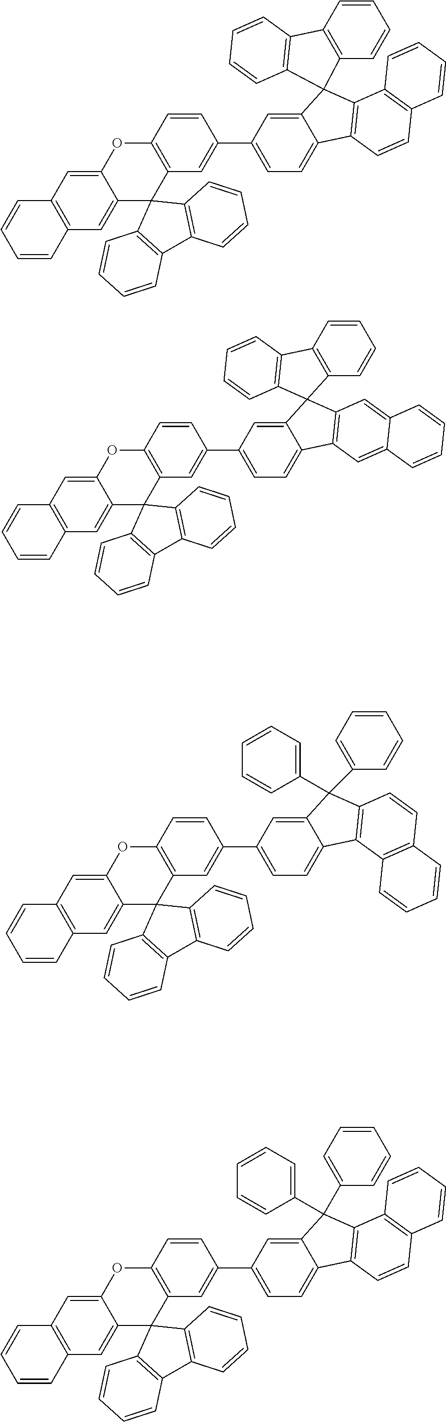 [SMILES: c1ccc2c(c1)-c1ccccc1C21c2cc(-c3ccc4c(c3)C3(c5ccccc5-c5ccccc53)c3c-4ccc4ccccc34)ccc2Oc2cc3ccccc3cc21 ; c1ccc2c(c1)-c1ccccc1C21c2cc(-c3ccc4c(c3)C3(c5ccccc5-c5ccccc53)c3cc5ccccc5cc3-4)ccc2Oc2cc3ccccc3cc21 ; c1ccc(C2(c3ccccc3)c3cc(-c4ccc5c(c4)C4(c6cc7ccccc7cc6O5)c5ccccc5-c5ccccc54)ccc3-c3c2ccc2ccccc32)cc1 ; c1ccc(C2(c3ccccc3)c3cc(-c4ccc5c(c4)C4(c6cc7ccccc7cc6O5)c5ccccc5-c5ccccc54)ccc3-c3ccc4ccccc4c32)cc1]

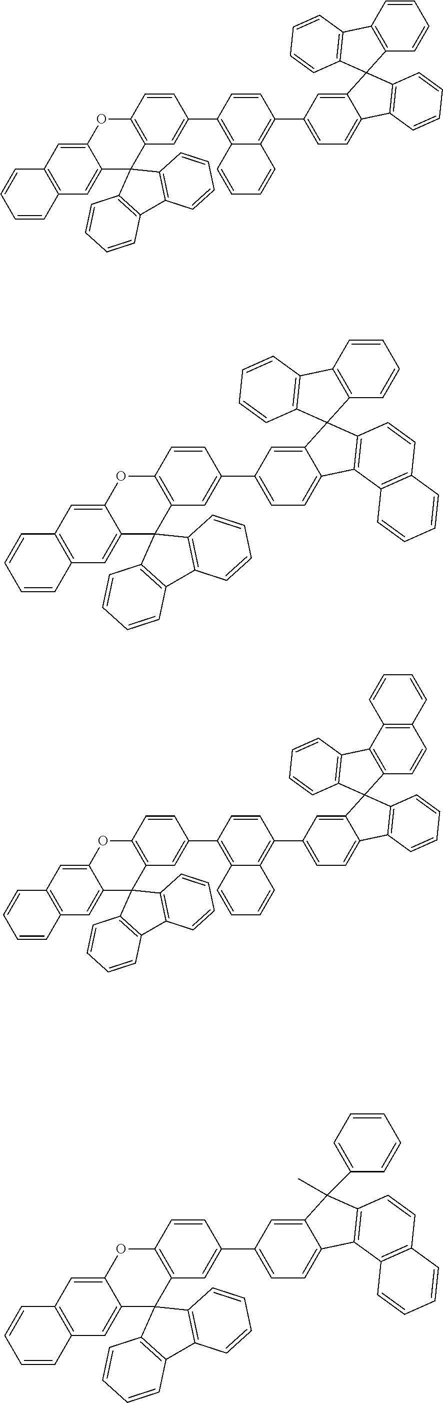 [SMILES: c1ccc2c(c1)-c1ccccc1C21c2cc(-c3ccc(-c4ccc5c(c4)C4(c6ccccc6-c6ccccc64)c4ccccc4-5)c4ccccc34)ccc2Oc2cc3ccccc3cc21 ; c1ccc2c(c1)-c1ccccc1C21c2cc(-c3ccc4c(c3)C3(c5ccccc5-c5ccccc53)c3ccc5ccccc5c3-4)ccc2Oc2cc3ccccc3cc21 ; c1ccc2c(c1)-c1ccccc1C21c2cc(-c3ccc(-c4ccc5c(c4)C4(c6ccccc6-5)c5ccccc5-c5c4ccc4ccccc54)c4ccccc34)ccc2Oc2cc3ccccc3cc21 ; CC1(c2ccccc2)c2cc(-c3ccc4c(c3)C3(c5cc6ccccc6cc5O4)c4ccccc4-c4ccccc43)ccc2-c2c1ccc1ccccc21]

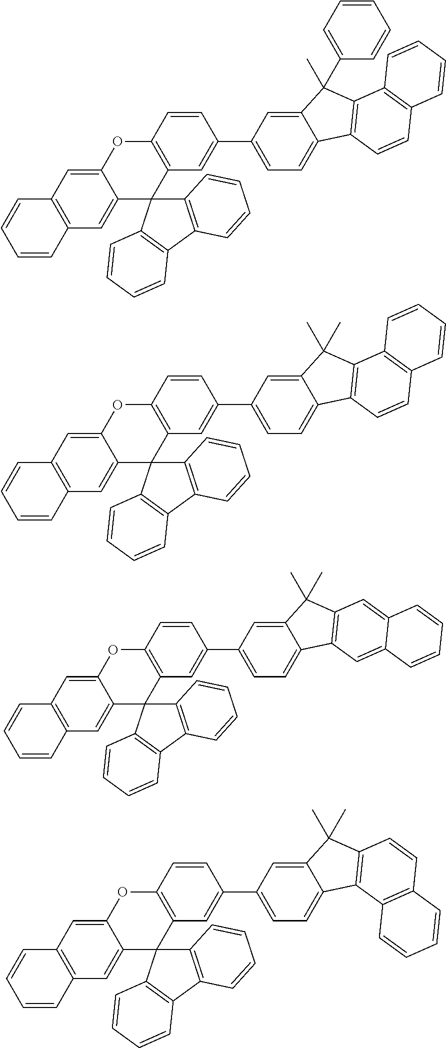 [SMILES: CC1(c2ccccc2)c2cc(-c3ccc4c(c3)C3(c5cc6ccccc6cc5O4)c4ccccc4-c4ccccc43)ccc2-c2ccc3ccccc3c21 ; CC1(C)c2cc(-c3ccc4c(c3)C3(c5cc6ccccc6cc5O4)c4ccccc4-c4ccccc43)ccc2-c2ccc3ccccc3c21 ; CC1(C)c2cc(-c3ccc4c(c3)C3(c5cc6ccccc6cc5O4)c4ccccc4-c4ccccc43)ccc2-c2cc3ccccc3cc21 ; CC1(C)c2cc(-c3ccc4c(c3)C3(c5cc6ccccc6cc5O4)c4ccccc4-c4ccccc43)ccc2-c2c1ccc1ccccc21]

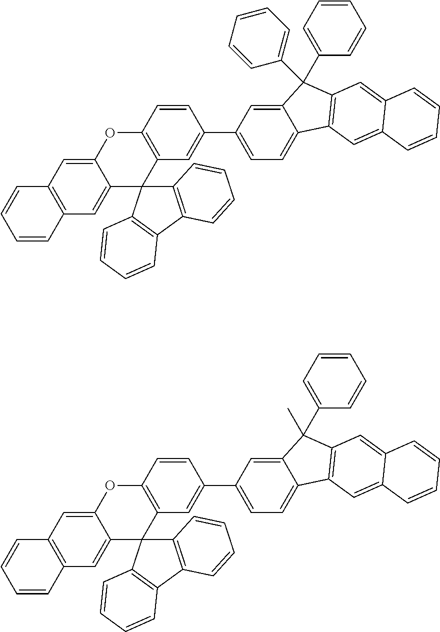 [SMILES: c1ccc(C2(c3ccccc3)c3cc(-c4ccc5c(c4)C4(c6cc7ccccc7cc6O5)c5ccccc5-c5ccccc54)ccc3-c3cc4ccccc4cc32)cc1 ; CC1(c2ccccc2)c2cc(-c3ccc4c(c3)C3(c5cc6ccccc6cc5O4)c4ccccc4-c4ccccc43)ccc2-c2cc3ccccc3cc21]

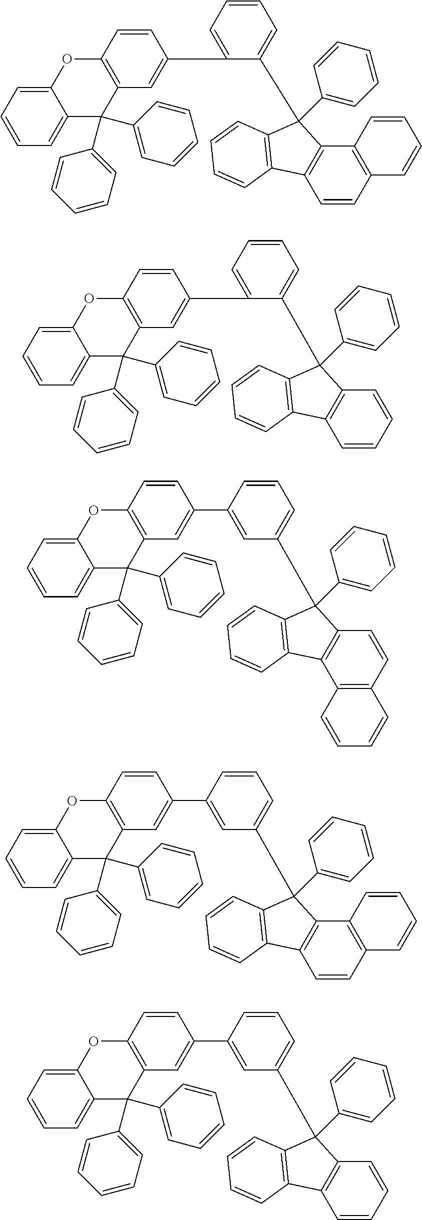

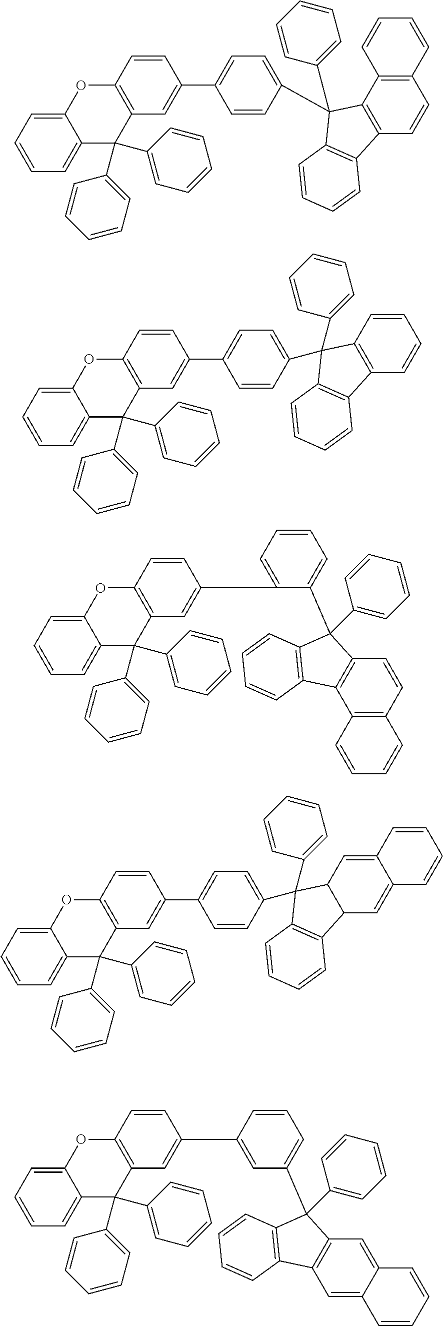

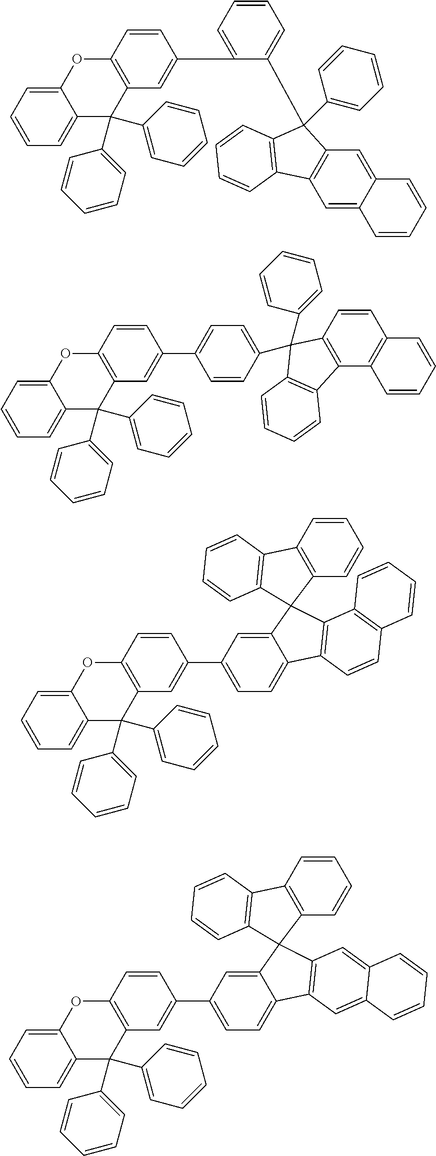 [SMILES: c1ccc(C2(c3ccccc3)c3ccccc3Oc3ccc(-c4ccccc4C4(c5ccccc5)c5ccccc5-c5cc6ccccc6cc54)cc32)cc1 ; c1ccc(C2(c3ccccc3)c3ccccc3Oc3ccc(-c4ccc(C5(c6ccccc6)c6ccccc6-c6c5ccc5ccccc65)cc4)cc32)cc1 ; c1ccc(C2(c3ccccc3)c3ccccc3Oc3ccc(-c4ccc5c(c4)C4(c6ccccc6-c6ccccc64)c4c-5ccc5ccccc45)cc32)cc1 ; c1ccc(C2(c3ccccc3)c3ccccc3Oc3ccc(-c4ccc5c(c4)C4(c6ccccc6-c6ccccc64)c4cc6ccccc6cc4-5)cc32)cc1]

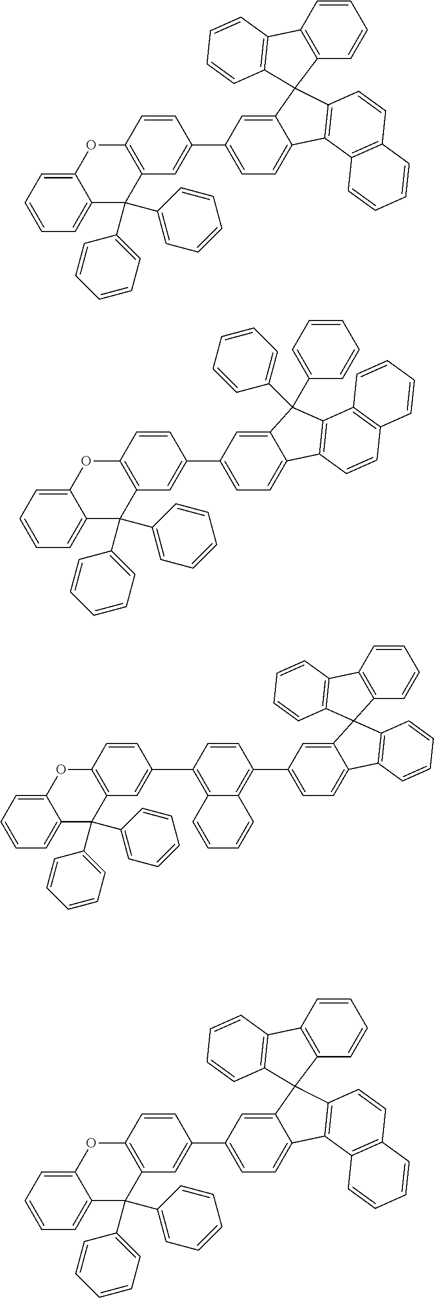 [SMILES: c1ccc(C2(c3ccccc3)c3ccccc3Oc3ccc(-c4ccc5c(c4)C4(c6ccccc6-c6ccccc64)c4ccc6ccccc6c4-5)cc32)cc1 ; c1ccc(C2(c3ccccc3)c3ccccc3Oc3ccc(-c4ccc5c(c4)C(c4ccccc4)(c4ccccc4)c4c-5ccc5ccccc45)cc32)cc1 ; c1ccc(C2(c3ccccc3)c3ccccc3Oc3ccc(-c4ccc(-c5ccc6c(c5)C5(c7ccccc7-c7ccccc75)c5ccccc5-6)c5ccccc45)cc32)cc1 ; c1ccc(C2(c3ccccc3)c3ccccc3Oc3ccc(-c4ccc5c(c4)C4(c6ccccc6-c6ccccc64)c4ccc6ccccc6c4-5)cc32)cc1]

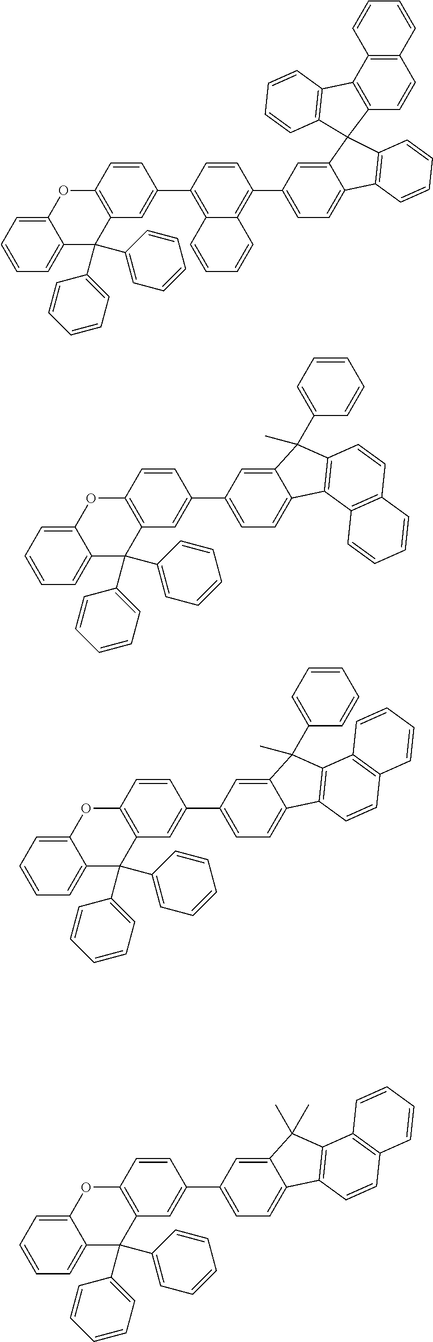 [SMILES: c1ccc(C2(c3ccccc3)c3ccccc3Oc3ccc(-c4ccc(-c5ccc6c(c5)C5(c7ccccc7-6)c6ccccc6-c6c5ccc5ccccc65)c5ccccc45)cc32)cc1 ; CC1(c2ccccc2)c2cc(-c3ccc4c(c3)C(c3ccccc3)(c3ccccc3)c3ccccc3O4)ccc2-c2c1ccc1ccccc21 ; CC1(c2ccccc2)c2cc(-c3ccc4c(c3)C(c3ccccc3)(c3ccccc3)c3ccccc3O4)ccc2-c2ccc3ccccc3c21 ; CC1(C)c2cc(-c3ccc4c(c3)C(c3ccccc3)(c3ccccc3)c3ccccc3O4)ccc2-c2ccc3ccccc3c21]

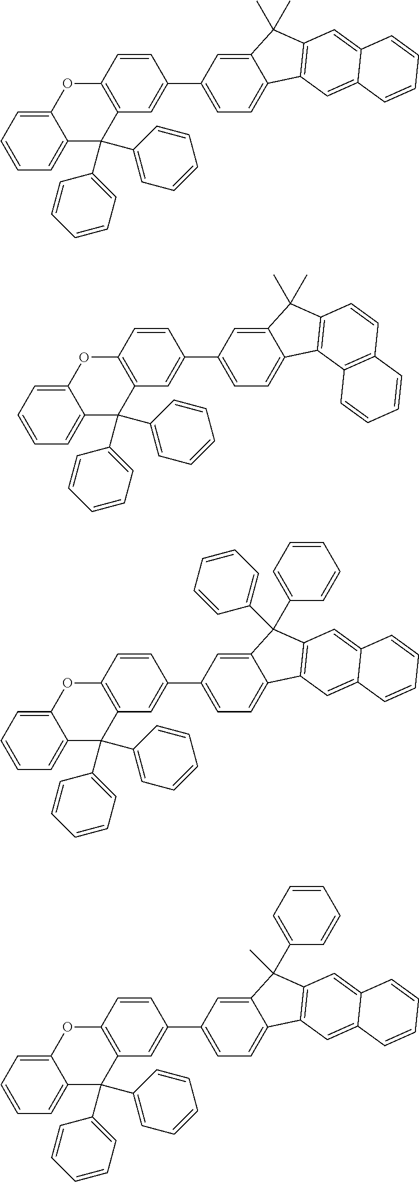 [SMILES: CC1(C)c2cc(-c3ccc4c(c3)C(c3ccccc3)(c3ccccc3)c3ccccc3O4)ccc2-c2cc3ccccc3cc21 ; CC1(C)c2cc(-c3ccc4c(c3)C(c3ccccc3)(c3ccccc3)c3ccccc3O4)ccc2-c2c1ccc1ccccc21 ; c1ccc(C2(c3ccccc3)c3ccccc3Oc3ccc(-c4ccc5c(c4)C(c4ccccc4)(c4ccccc4)c4cc6ccccc6cc4-5)cc32)cc1 ; CC1(c2ccccc2)c2cc(-c3ccc4c(c3)C(c3ccccc3)(c3ccccc3)c3ccccc3O4)ccc2-c2cc3ccccc3cc21]

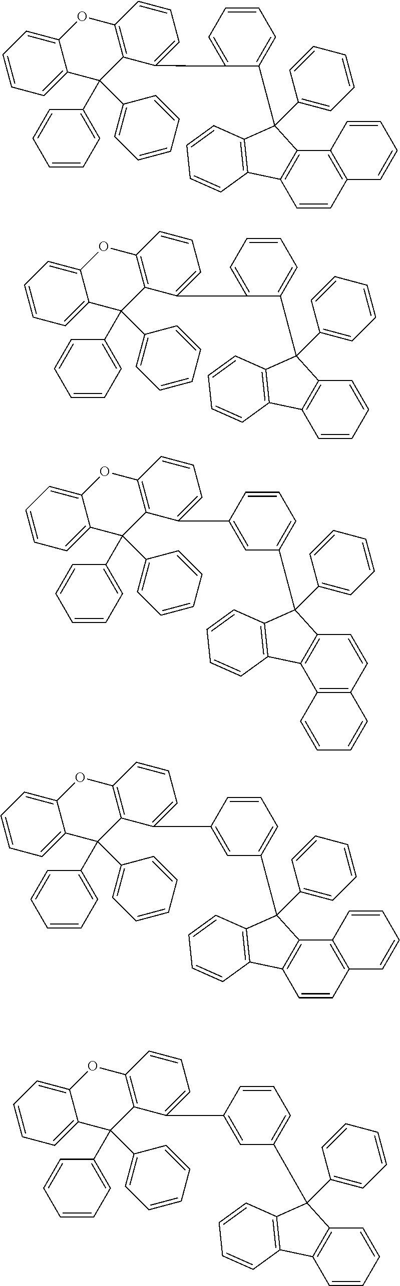

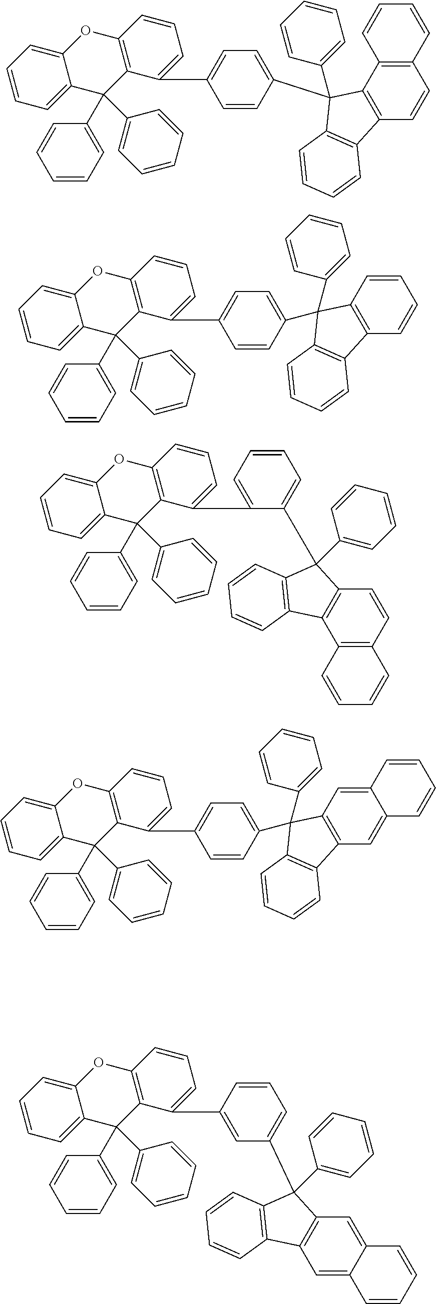

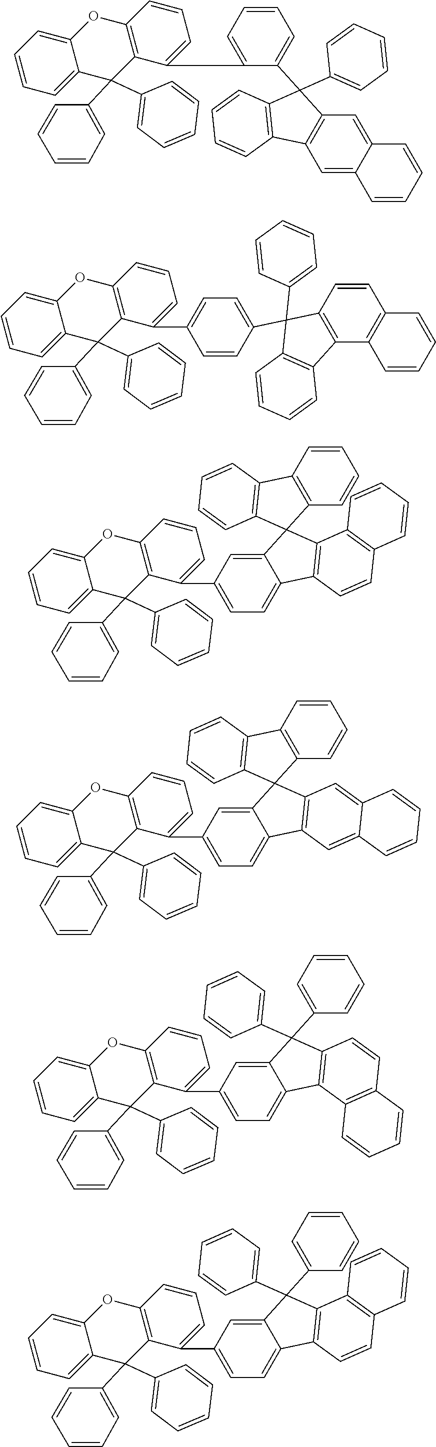

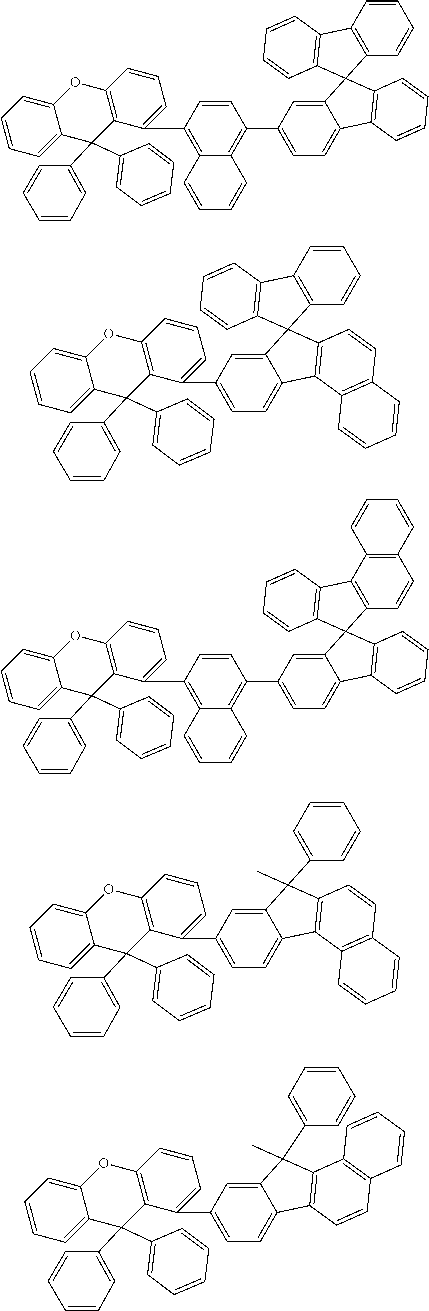 [SMILES: c1ccc(C2(c3ccccc3)c3ccccc3Oc3cccc(-c4ccc(-c5ccc6c(c5)C5(c7ccccc7-c7ccccc75)c5ccccc5-6)c5ccccc45)c32)cc1 ; c1ccc(C2(c3ccccc3)c3ccccc3Oc3cccc(-c4ccc5c(c4)C4(c6ccccc6-c6ccccc64)c4ccc6ccccc6c4-5)c32)cc1 ; c1ccc(C2(c3ccccc3)c3ccccc3Oc3cccc(-c4ccc(-c5ccc6c(c5)C5(c7ccccc7-6)c6ccccc6-c6c5ccc5ccccc65)c5ccccc45)c32)cc1 ; CC1(c2ccccc2)c2cc(-c3cccc4c3C(c3ccccc3)(c3ccccc3)c3ccccc3O4)ccc2-c2c1ccc1ccccc21 ; CC1(c2ccccc2)c2cc(-c3cccc4c3C(c3ccccc3)(c3ccccc3)c3ccccc3O4)ccc2-c2ccc3ccccc3c21]

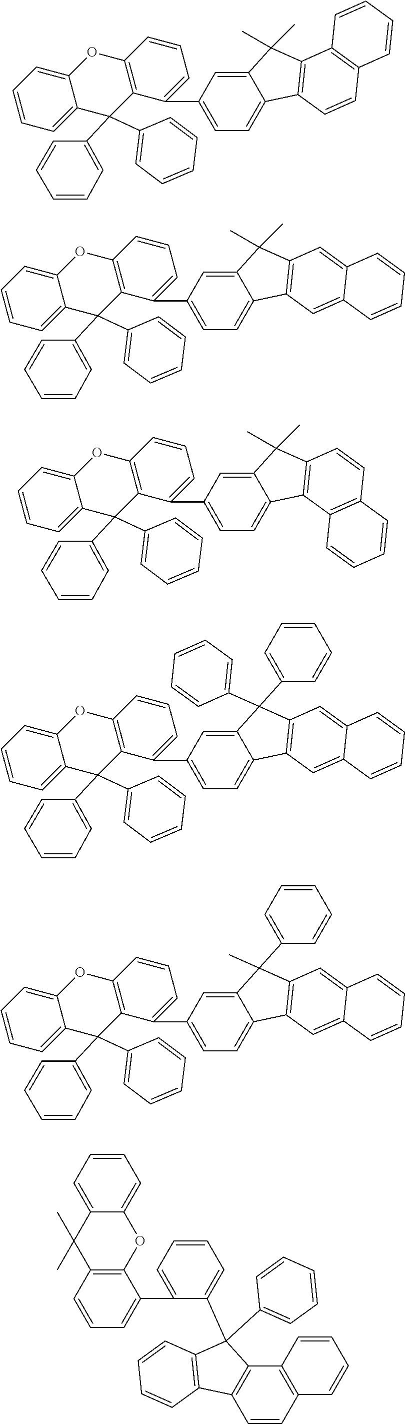

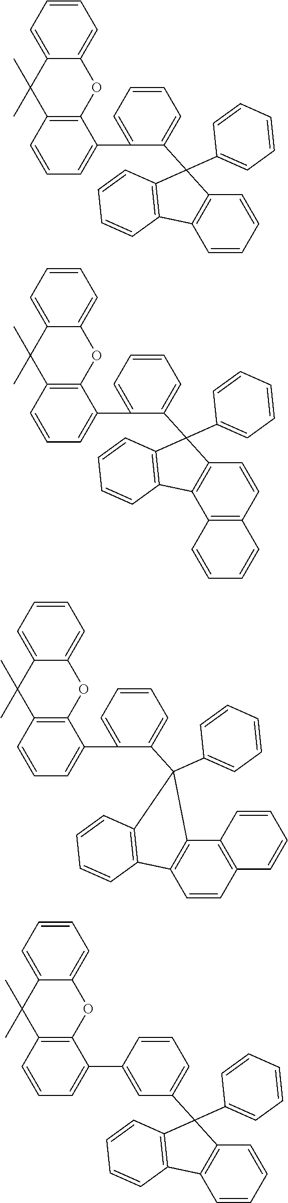 [SMILES: CC1(C)c2ccccc2Oc2c(-c3ccccc3C3(c4ccccc4)c4ccccc4-c4ccccc43)cccc21 ; CC1(C)c2ccccc2Oc2c(-c3ccccc3C3(c4ccccc4)c4ccccc4-c4c3ccc3ccccc43)cccc21 ; CC1(C)c2ccccc2Oc2c(-c3ccccc3C3(c4ccccc4)c4ccccc4-c4ccc5ccccc5c43)cccc21 ; CC1(C)c2ccccc2Oc2c(-c3cccc(C4(c5ccccc5)c5ccccc5-c5ccccc54)c3)cccc21]

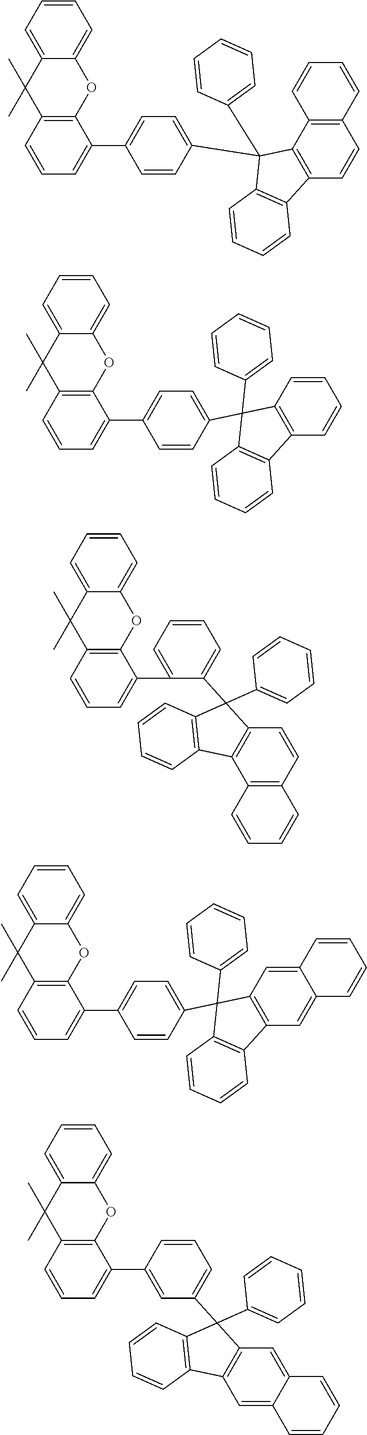 [SMILES: CC1(C)c2ccccc2Oc2c(-c3ccc(C4(c5ccccc5)c5ccccc5-c5ccc6ccccc6c54)cc3)cccc21 ; CC1(C)c2ccccc2Oc2c(-c3ccc(C4(c5ccccc5)c5ccccc5-c5ccccc54)cc3)cccc21 ; CC1(C)c2ccccc2Oc2c(-c3ccccc3C3(c4ccccc4)c4ccccc4-c4c3ccc3ccccc43)cccc21 ; CC1(C)c2ccccc2Oc2c(-c3ccc(C4(c5ccccc5)c5ccccc5-c5cc6ccccc6cc54)cc3)cccc21 ; CC1(C)c2ccccc2Oc2c(-c3cccc(C4(c5ccccc5)c5ccccc5-c5cc6ccccc6cc54)c3)cccc21]

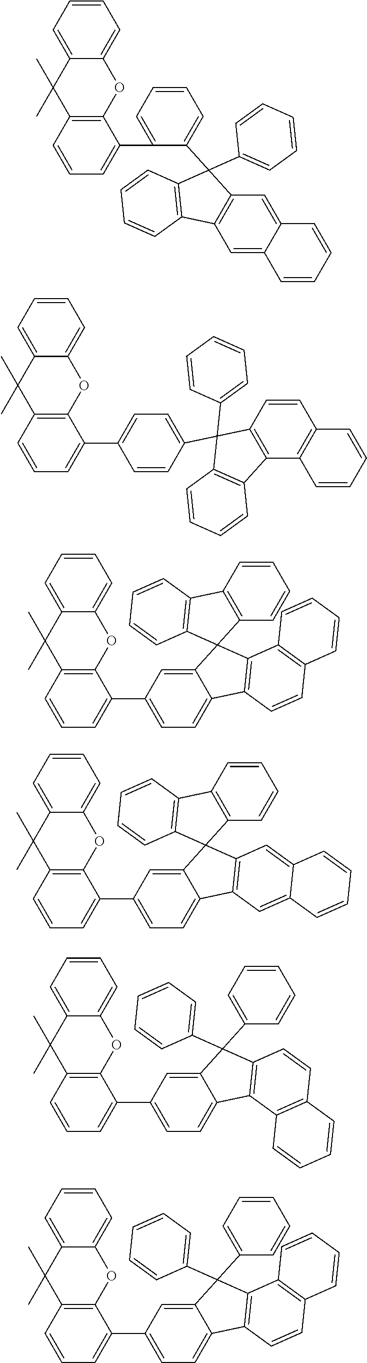

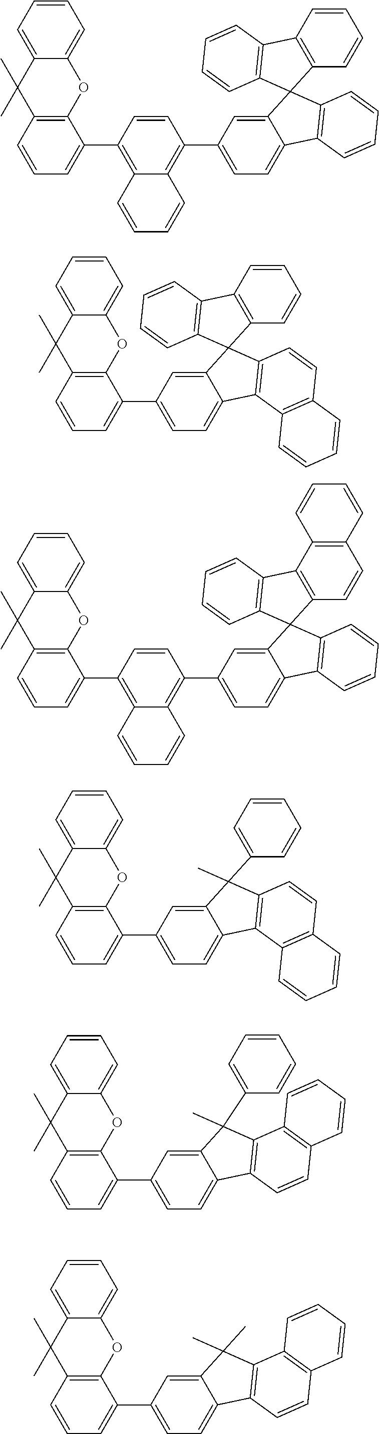

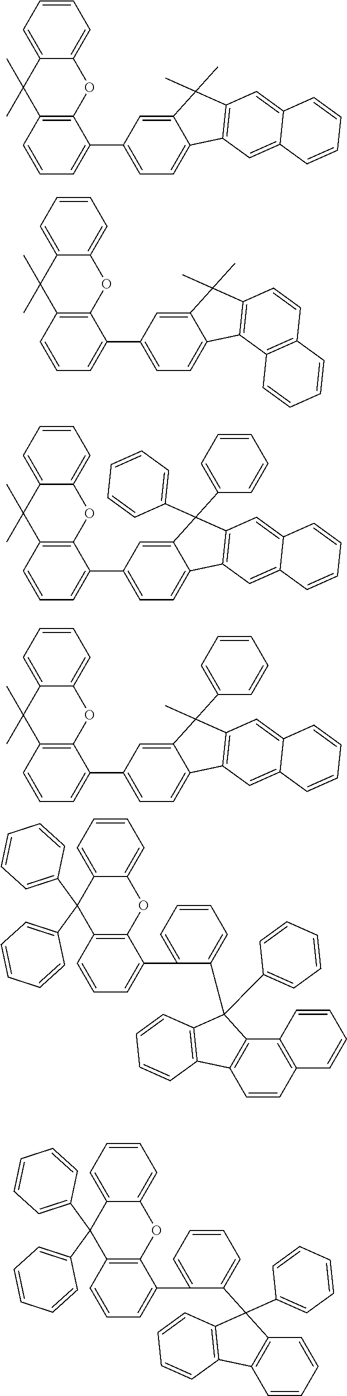

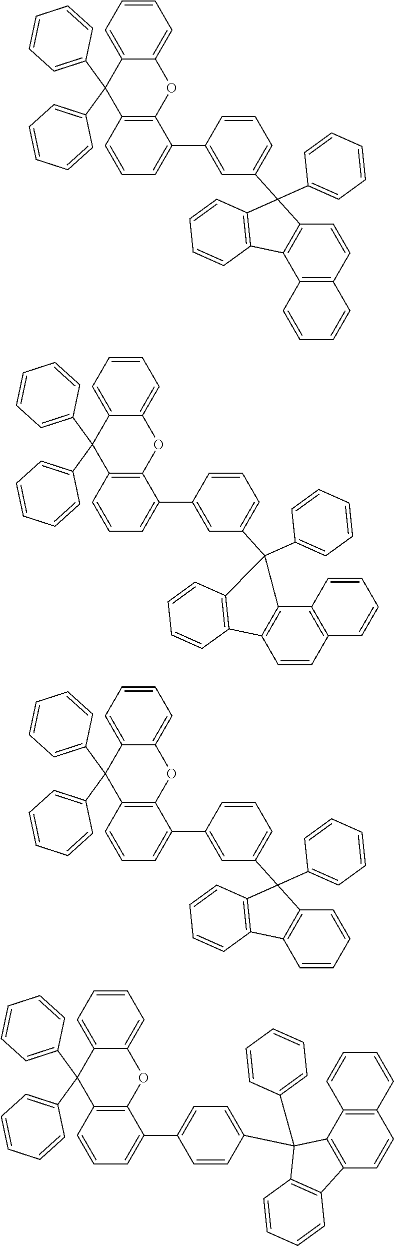 [SMILES: c1ccc(C2(c3ccccc3)c3ccccc3Oc3c(-c4cccc(C5(c6ccccc6)c6ccccc6-c6c5ccc5ccccc65)c4)cccc32)cc1 ; c1ccc(C2(c3ccccc3)c3ccccc3Oc3c(-c4cccc(C5(c6ccccc6)c6ccccc6-c6ccc7ccccc7c65)c4)cccc32)cc1 ; c1ccc(C2(c3cccc(-c4cccc5c4Oc4ccccc4C5(c4ccccc4)c4ccccc4)c3)c3ccccc3-c3ccccc32)cc1 ; c1ccc(C2(c3ccccc3)c3ccccc3Oc3c(-c4ccc(C5(c6ccccc6)c6ccccc6-c6ccc7ccccc7c65)cc4)cccc32)cc1]

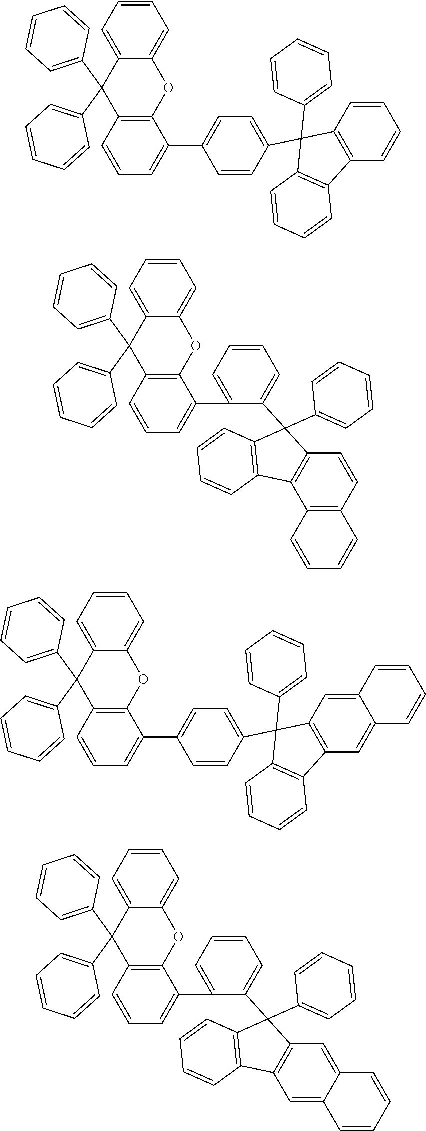 [SMILES: c1ccc(C2(c3ccc(-c4cccc5c4Oc4ccccc4C5(c4ccccc4)c4ccccc4)cc3)c3ccccc3-c3ccccc32)cc1 ; c1ccc(C2(c3ccccc3)c3ccccc3Oc3c(-c4ccccc4C4(c5ccccc5)c5ccccc5-c5c4ccc4ccccc54)cccc32)cc1 ; c1ccc(C2(c3ccc(-c4cccc5c4Oc4ccccc4C5(c4ccccc4)c4ccccc4)cc3)c3ccccc3-c3cc4ccccc4cc32)cc1 ; c1ccc(C2(c3ccccc3)c3ccccc3Oc3c(-c4ccccc4C4(c5ccccc5)c5ccccc5-c5cc6ccccc6cc54)cccc32)cc1]

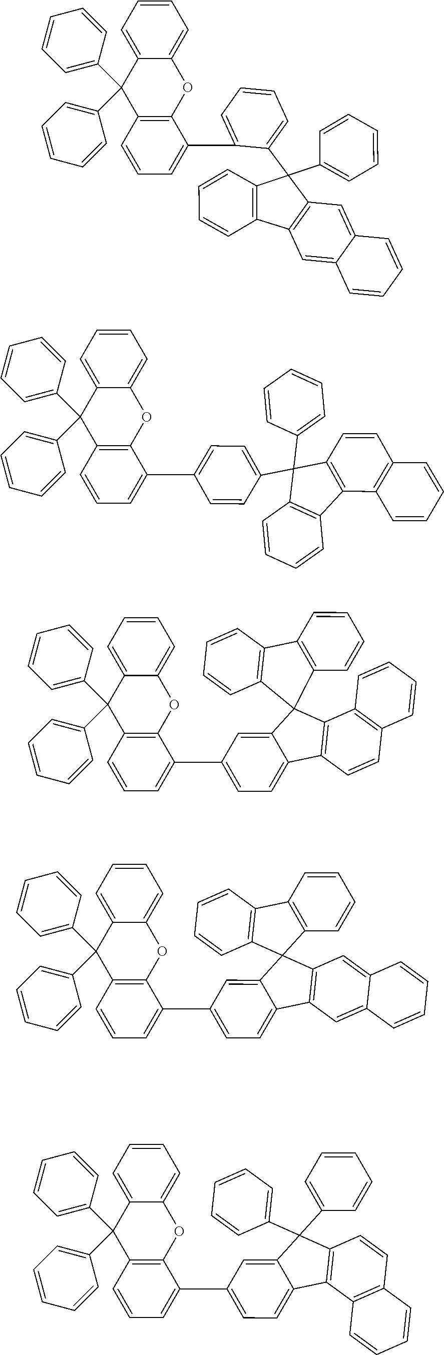 [SMILES: c1ccc(C2(c3ccccc3)c3ccccc3Oc3c(-c4ccccc4C4(c5ccccc5)c5ccccc5-c5cc6ccccc6cc54)cccc32)cc1 ; c1ccc(C2(c3ccccc3)c3ccccc3Oc3c(-c4ccc(C5(c6ccccc6)c6ccccc6-c6c5ccc5ccccc65)cc4)cccc32)cc1 ; c1ccc(C2(c3ccccc3)c3ccccc3Oc3c(-c4ccc5c(c4)C4(c6ccccc6-c6ccccc64)c4c-5ccc5ccccc45)cccc32)cc1 ; c1ccc(C2(c3ccccc3)c3ccccc3Oc3c(-c4ccc5c(c4)C4(c6ccccc6-c6ccccc64)c4cc6ccccc6cc4-5)cccc32)cc1 ; c1ccc(C2(c3ccccc3)c3ccccc3Oc3c(-c4ccc5c(c4)C(c4ccccc4)(c4ccccc4)c4ccc6ccccc6c4-5)cccc32)cc1]

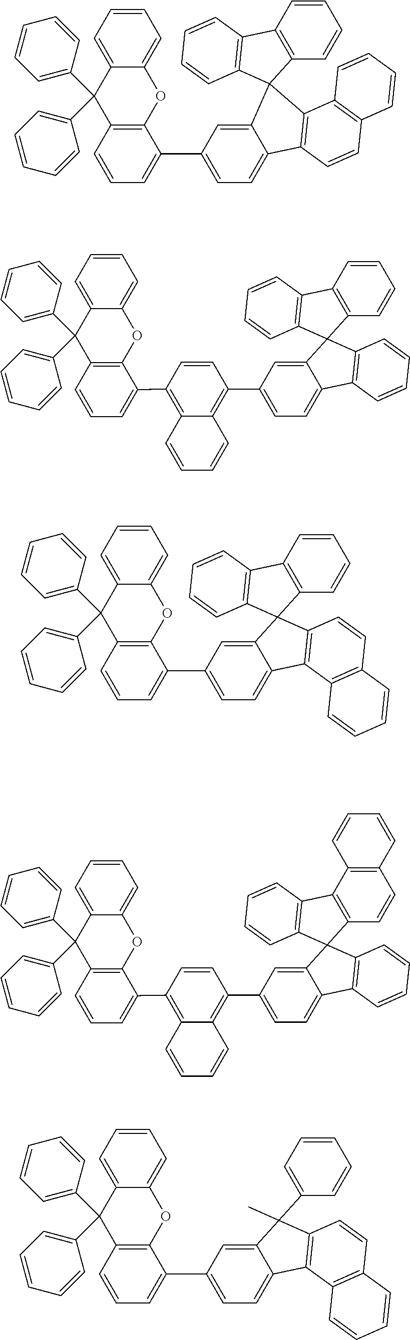 [SMILES: c1ccc(C2(c3ccccc3)c3ccccc3Oc3c(-c4ccc5c(c4)C4(c6ccccc6-c6ccccc64)c4c-5ccc5ccccc45)cccc32)cc1 ; c1ccc(C2(c3ccccc3)c3ccccc3Oc3c(-c4ccc(-c5ccc6c(c5)C5(c7ccccc7-c7ccccc75)c5ccccc5-6)c5ccccc45)cccc32)cc1 ; c1ccc(C2(c3ccccc3)c3ccccc3Oc3c(-c4ccc5c(c4)C4(c6ccccc6-c6ccccc64)c4ccc6ccccc6c4-5)cccc32)cc1 ; c1ccc(C2(c3ccccc3)c3ccccc3Oc3c(-c4ccc(-c5ccc6c(c5)C5(c7ccccc7-6)c6ccccc6-c6c5ccc5ccccc65)c5ccccc45)cccc32)cc1 ; CC1(c2ccccc2)c2cc(-c3cccc4c3Oc3ccccc3C4(c3ccccc3)c3ccccc3)ccc2-c2c1ccc1ccccc21]

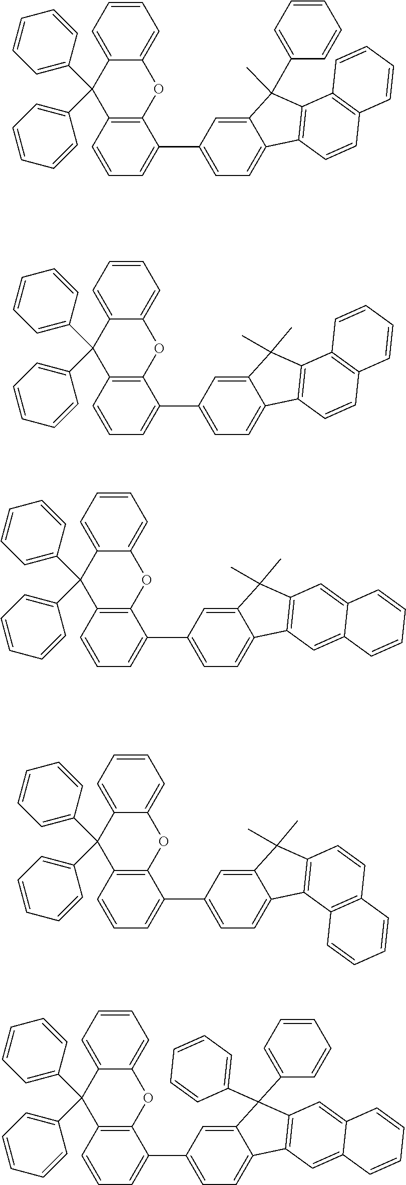 [SMILES: CC1(c2ccccc2)c2cc(-c3cccc4c3Oc3ccccc3C4(c3ccccc3)c3ccccc3)ccc2-c2ccc3ccccc3c21 ; CC1(C)c2cc(-c3cccc4c3Oc3ccccc3C4(c3ccccc3)c3ccccc3)ccc2-c2ccc3ccccc3c21 ; CC1(C)c2cc(-c3cccc4c3Oc3ccccc3C4(c3ccccc3)c3ccccc3)ccc2-c2cc3ccccc3cc21 ; CC1(C)c2cc(-c3cccc4c3Oc3ccccc3C4(c3ccccc3)c3ccccc3)ccc2-c2c1ccc1ccccc21 ; c1ccc(C2(c3ccccc3)c3cc(-c4cccc5c4Oc4ccccc4C5(c4ccccc4)c4ccccc4)ccc3-c3cc4ccccc4cc32)cc1]

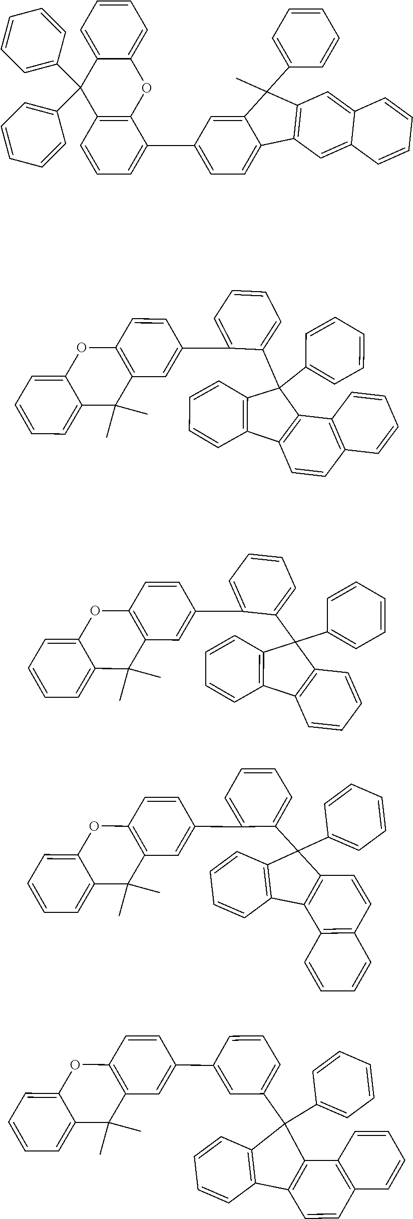 [SMILES: CC1(c2ccccc2)c2cc(-c3cccc4c3Oc3ccccc3C4(c3ccccc3)c3ccccc3)ccc2-c2cc3ccccc3cc21 ; CC1(C)c2ccccc2Oc2ccc(-c3ccccc3C3(c4ccccc4)c4ccccc4-c4ccc5ccccc5c43)cc21 ; CC1(C)c2ccccc2Oc2ccc(-c3ccccc3C3(c4ccccc4)c4ccccc4-c4ccccc43)cc21 ; CC1(C)c2ccccc2Oc2ccc(-c3ccccc3C3(c4ccccc4)c4ccccc4-c4c3ccc3ccccc43)cc21 ; CC1(C)c2ccccc2Oc2ccc(-c3cccc(C4(c5ccccc5)c5ccccc5-c5ccc6ccccc6c54)c3)cc21]

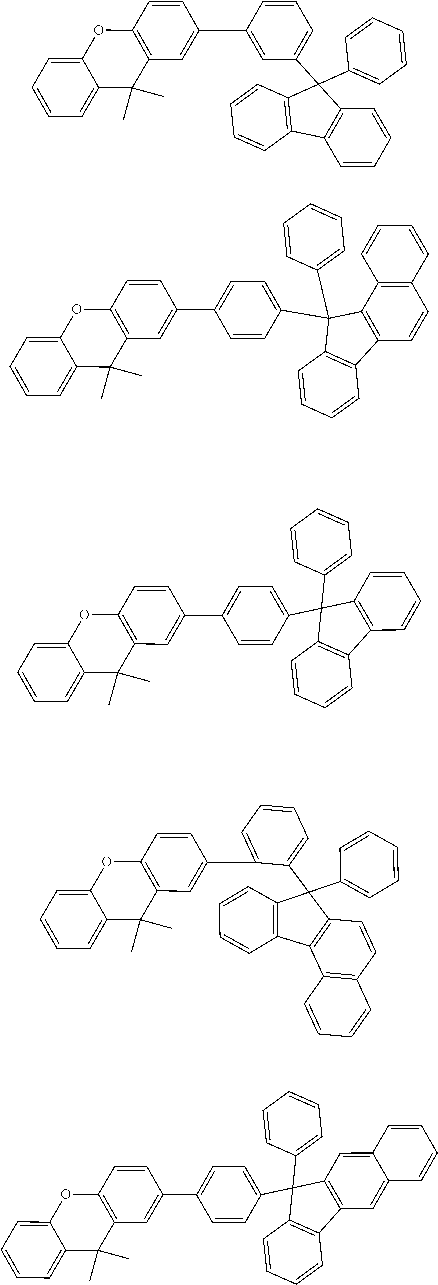 [SMILES: CC1(C)c2ccccc2Oc2ccc(-c3cccc(C4(c5ccccc5)c5ccccc5-c5ccccc54)c3)cc21 ; CC1(C)c2ccccc2Oc2ccc(-c3ccc(C4(c5ccccc5)c5ccccc5-c5ccc6ccccc6c54)cc3)cc21 ; CC1(C)c2ccccc2Oc2ccc(-c3ccc(C4(c5ccccc5)c5ccccc5-c5ccccc54)cc3)cc21 ; CC1(C)c2ccccc2Oc2ccc(-c3ccccc3C3(c4ccccc4)c4ccccc4-c4c3ccc3ccccc43)cc21 ; CC1(C)c2ccccc2Oc2ccc(-c3ccc(C4(c5ccccc5)c5ccccc5-c5cc6ccccc6cc54)cc3)cc21]

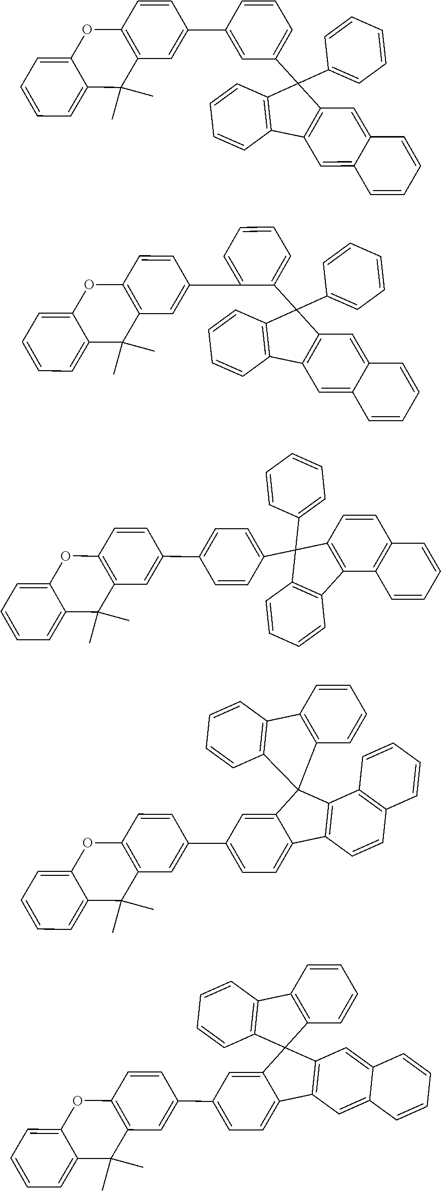

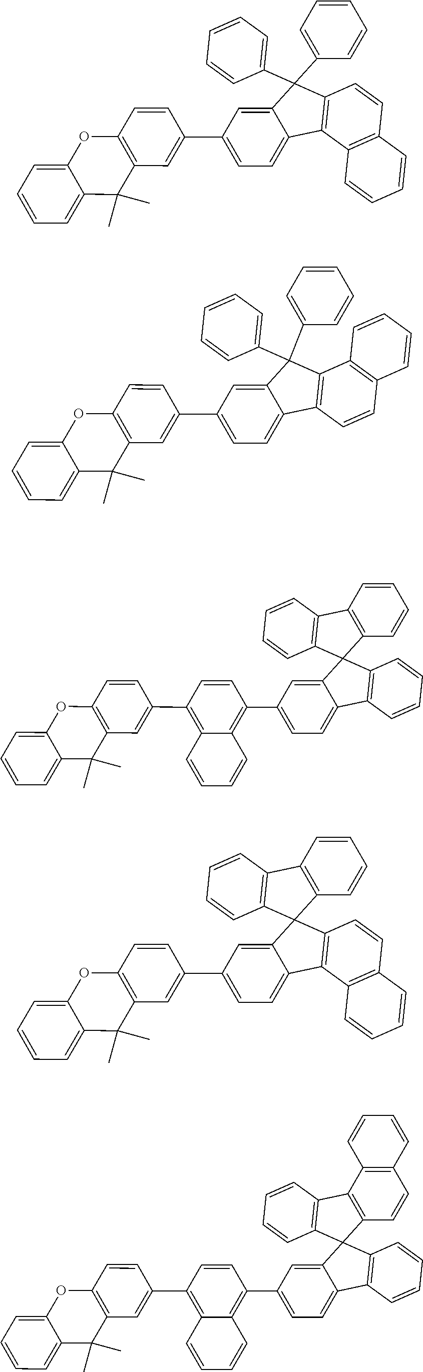 [SMILES: CC1(C)c2ccccc2Oc2ccc(-c3ccc4c(c3)C(c3ccccc3)(c3ccccc3)c3ccc5ccccc5c3-4)cc21 ; CC1(C)c2ccccc2Oc2ccc(-c3ccc4c(c3)C(c3ccccc3)(c3ccccc3)c3c-4ccc4ccccc34)cc21 ; CC1(C)c2ccccc2Oc2ccc(-c3ccc(-c4ccc5c(c4)C4(c6ccccc6-c6ccccc64)c4ccccc4-5)c4ccccc34)cc21 ; CC1(C)c2ccccc2Oc2ccc(-c3ccc4c(c3)C3(c5ccccc5-c5ccccc53)c3ccc5ccccc5c3-4)cc21 ; CC1(C)c2ccccc2Oc2ccc(-c3ccc(-c4ccc5c(c4)C4(c6ccccc6-5)c5ccccc5-c5c4ccc4ccccc54)c4ccccc34)cc21]

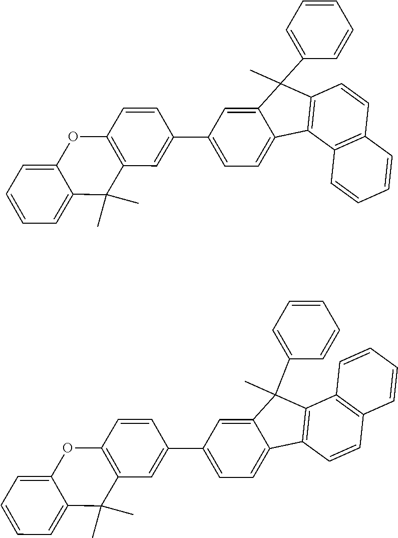 [SMILES: CC1(C)c2ccccc2Oc2ccc(-c3ccc4c(c3)C(C)(c3ccccc3)c3ccc5ccccc5c3-4)cc21 ; CC1(C)c2ccccc2Oc2ccc(-c3ccc4c(c3)C(C)(c3ccccc3)c3c-4ccc4ccccc34)cc21]

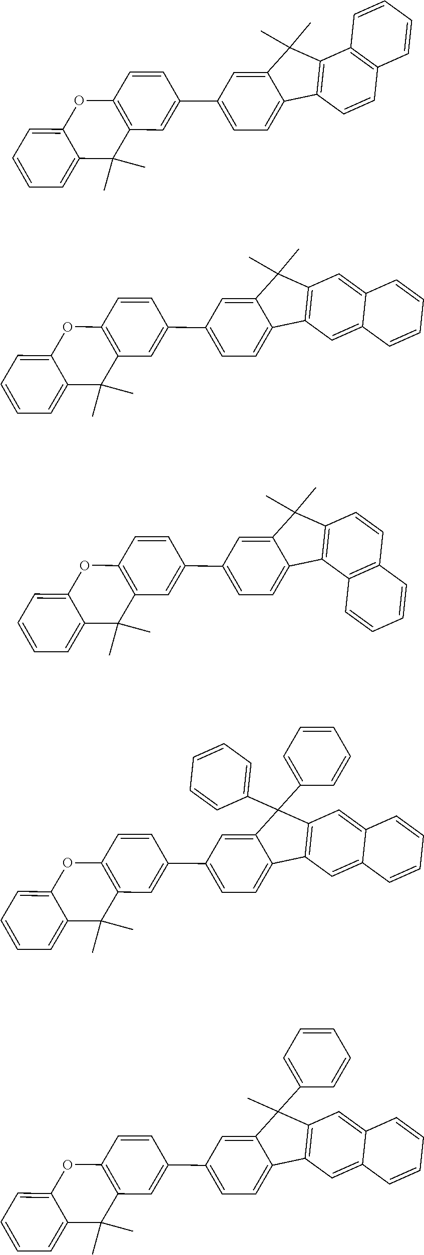 [SMILES: CC1(C)c2ccccc2Oc2ccc(-c3ccc4c(c3)C(C)(C)c3c-4ccc4ccccc34)cc21 ; CC1(C)c2ccccc2Oc2ccc(-c3ccc4c(c3)C(C)(C)c3cc5ccccc5cc3-4)cc21 ; CC1(C)c2ccccc2Oc2ccc(-c3ccc4c(c3)C(C)(C)c3ccc5ccccc5c3-4)cc21 ; CC1(C)c2ccccc2Oc2ccc(-c3ccc4c(c3)C(c3ccccc3)(c3ccccc3)c3cc5ccccc5cc3-4)cc21 ; CC1(C)c2ccccc2Oc2ccc(-c3ccc4c(c3)C(C)(c3ccccc3)c3cc5ccccc5cc3-4)cc21]

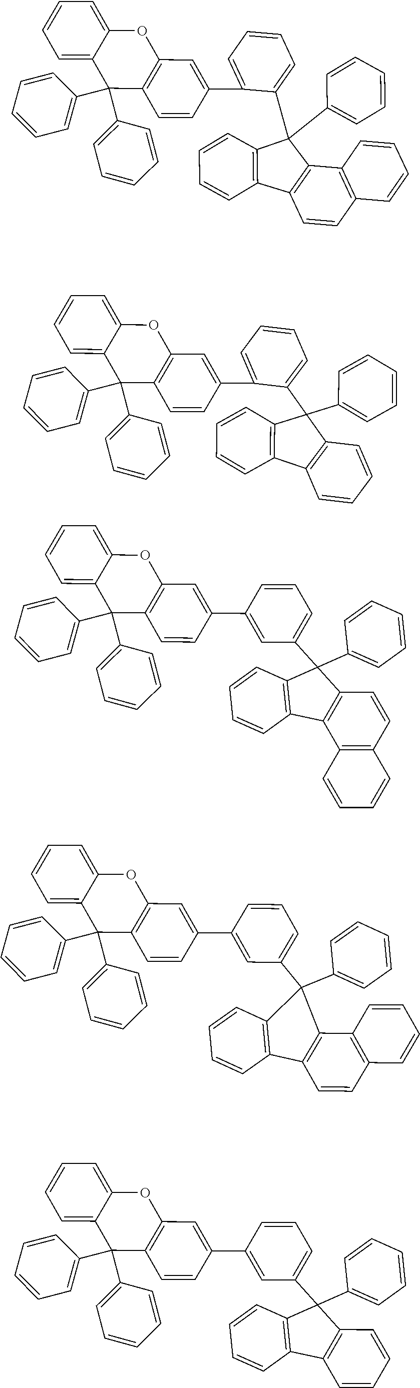 [SMILES: c1ccc(C2(c3ccccc3)c3ccccc3Oc3cc(-c4ccccc4C4(c5ccccc5)c5ccccc5-c5ccc6ccccc6c54)ccc32)cc1 ; c1ccc(C2(c3ccccc3)c3ccccc3Oc3cc(-c4ccccc4C4(c5ccccc5)c5ccccc5-c5ccccc54)ccc32)cc1 ; c1ccc(C2(c3ccccc3)c3ccccc3Oc3cc(-c4cccc(C5(c6ccccc6)c6ccccc6-c6c5ccc5ccccc65)c4)ccc32)cc1 ; c1ccc(C2(c3ccccc3)c3ccccc3Oc3cc(-c4cccc(C5(c6ccccc6)c6ccccc6-c6ccc7ccccc7c65)c4)ccc32)cc1 ; c1ccc(C2(c3ccccc3)c3ccccc3Oc3cc(-c4cccc(C5(c6ccccc6)c6ccccc6-c6ccccc65)c4)ccc32)cc1]

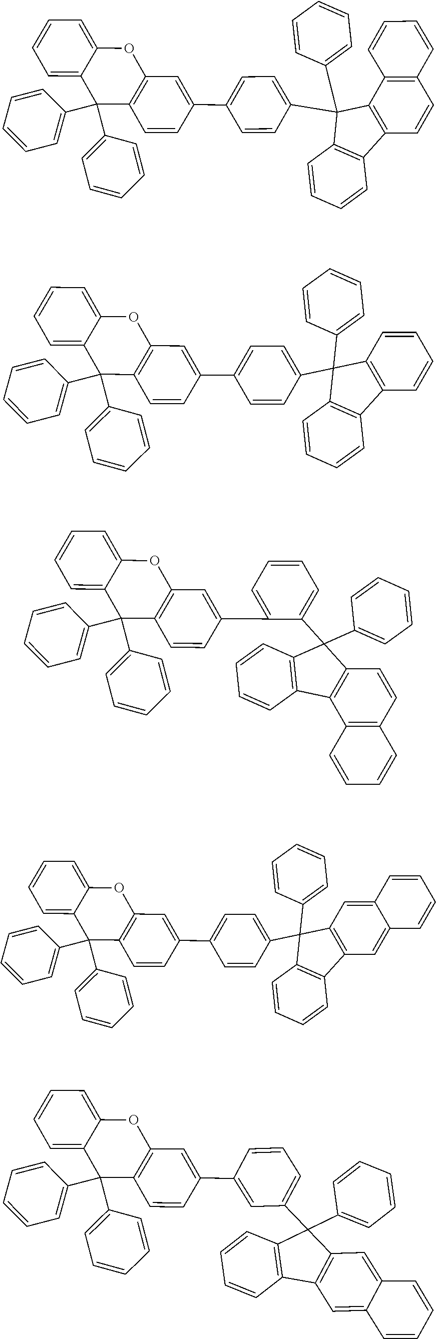 [SMILES: c1ccc(C2(c3ccccc3)c3ccccc3Oc3cc(-c4ccc(C5(c6ccccc6)c6ccccc6-c6ccc7ccccc7c65)cc4)ccc32)cc1 ; c1ccc(C2(c3ccccc3)c3ccccc3Oc3cc(-c4ccc(C5(c6ccccc6)c6ccccc6-c6ccccc65)cc4)ccc32)cc1 ; c1ccc(C2(c3ccccc3)c3ccccc3Oc3cc(-c4ccccc4C4(c5ccccc5)c5ccccc5-c5c4ccc4ccccc54)ccc32)cc1 ; c1ccc(C2(c3ccccc3)c3ccccc3Oc3cc(-c4ccc(C5(c6ccccc6)c6ccccc6-c6cc7ccccc7cc65)cc4)ccc32)cc1 ; c1ccc(C2(c3ccccc3)c3ccccc3Oc3cc(-c4cccc(C5(c6ccccc6)c6ccccc6-c6cc7ccccc7cc65)c4)ccc32)cc1]

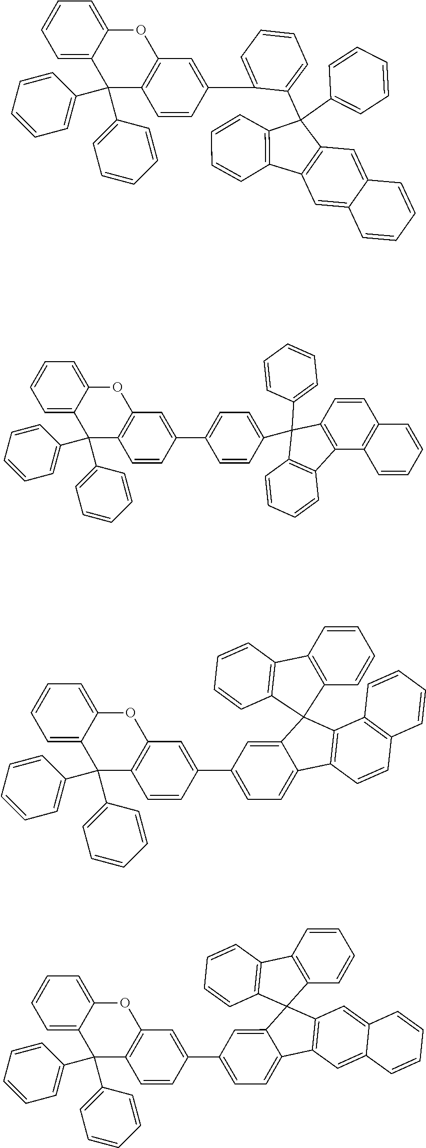 [SMILES: c1ccc(C2(c3ccccc3)c3ccccc3Oc3cc(-c4ccccc4C4(c5ccccc5)c5ccccc5-c5cc6ccccc6cc54)ccc32)cc1 ; c1ccc(C2(c3ccccc3)c3ccccc3Oc3cc(-c4ccc(C5(c6ccccc6)c6ccccc6-c6c5ccc5ccccc65)cc4)ccc32)cc1 ; c1ccc(C2(c3ccccc3)c3ccccc3Oc3cc(-c4ccc5c(c4)C4(c6ccccc6-c6ccccc64)c4c-5ccc5ccccc45)ccc32)cc1 ; c1ccc(C2(c3ccccc3)c3ccccc3Oc3cc(-c4ccc5c(c4)C4(c6ccccc6-c6ccccc64)c4cc6ccccc6cc4-5)ccc32)cc1]

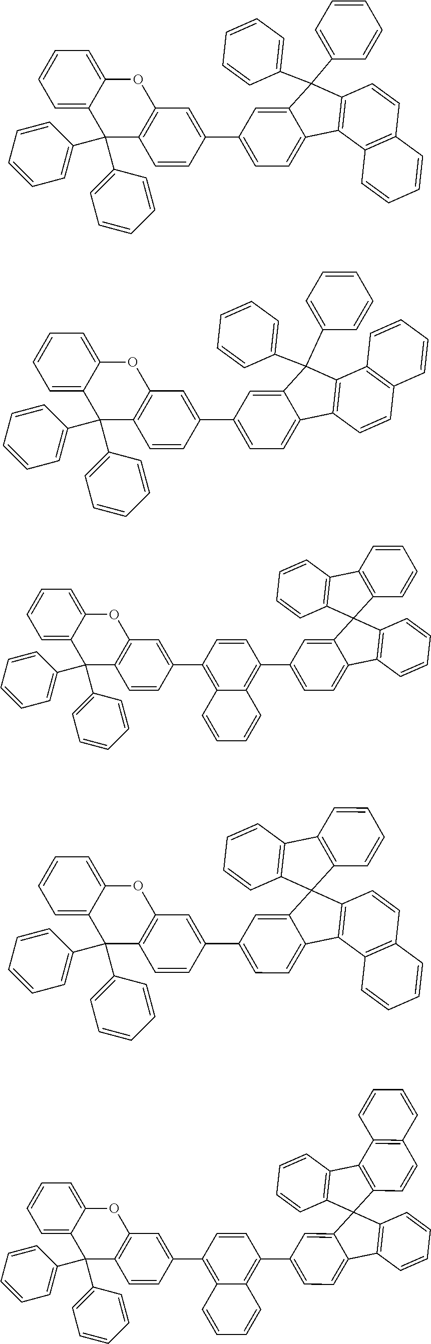 [SMILES: c1ccc(C2(c3ccccc3)c3ccccc3Oc3cc(-c4ccc5c(c4)C(c4ccccc4)(c4ccccc4)c4ccc6ccccc6c4-5)ccc32)cc1 ; c1ccc(C2(c3ccccc3)c3ccccc3Oc3cc(-c4ccc5c(c4)C(c4ccccc4)(c4ccccc4)c4c-5ccc5ccccc45)ccc32)cc1 ; c1ccc(C2(c3ccccc3)c3ccccc3Oc3cc(-c4ccc(-c5ccc6c(c5)C5(c7ccccc7-c7ccccc75)c5ccccc5-6)c5ccccc45)ccc32)cc1 ; c1ccc(C2(c3ccccc3)c3ccccc3Oc3cc(-c4ccc5c(c4)C4(c6ccccc6-c6ccccc64)c4ccc6ccccc6c4-5)ccc32)cc1 ; c1ccc(C2(c3ccccc3)c3ccccc3Oc3cc(-c4ccc(-c5ccc6c(c5)C5(c7ccccc7-6)c6ccccc6-c6c5ccc5ccccc65)c5ccccc45)ccc32)cc1]

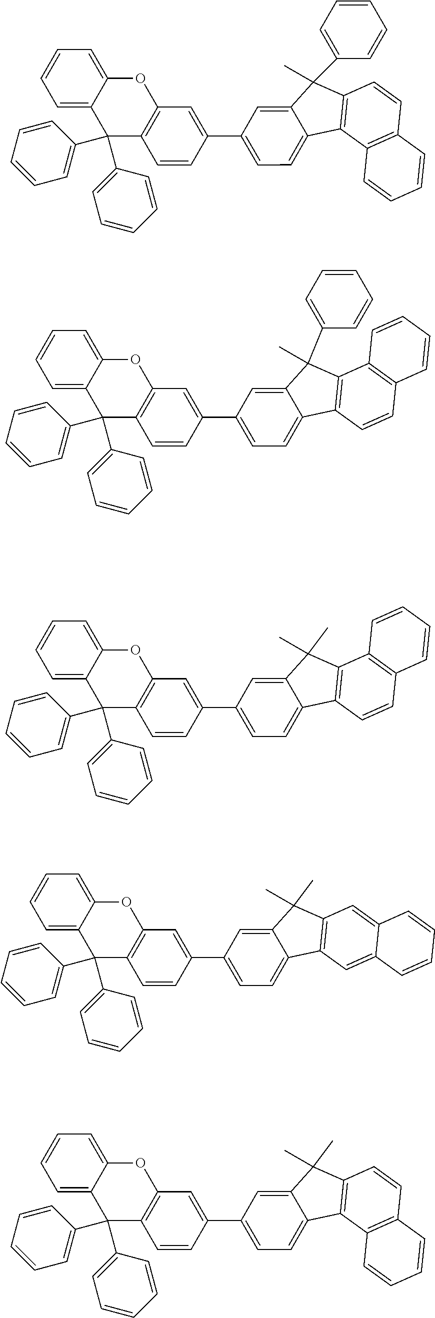 [SMILES: CC1(c2ccccc2)c2cc(-c3ccc4c(c3)Oc3ccccc3C4(c3ccccc3)c3ccccc3)ccc2-c2c1ccc1ccccc21 ; CC1(c2ccccc2)c2cc(-c3ccc4c(c3)Oc3ccccc3C4(c3ccccc3)c3ccccc3)ccc2-c2ccc3ccccc3c21 ; CC1(C)c2cc(-c3ccc4c(c3)Oc3ccccc3C4(c3ccccc3)c3ccccc3)ccc2-c2ccc3ccccc3c21 ; CC1(C)c2cc(-c3ccc4c(c3)Oc3ccccc3C4(c3ccccc3)c3ccccc3)ccc2-c2cc3ccccc3cc21 ; CC1(C)c2cc(-c3ccc4c(c3)Oc3ccccc3C4(c3ccccc3)c3ccccc3)ccc2-c2c1ccc1ccccc21]

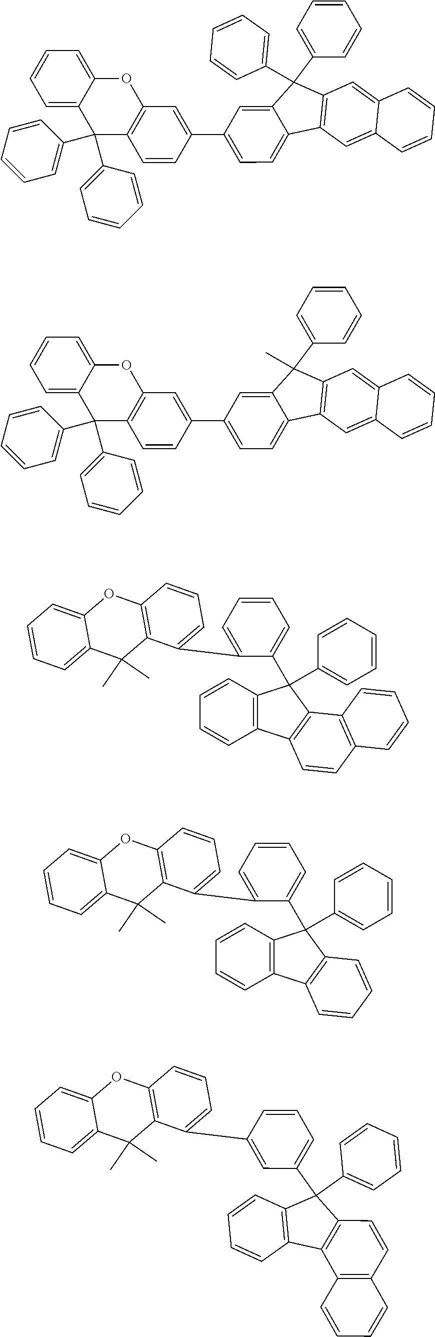 [SMILES: c1ccc(C2(c3ccccc3)c3ccccc3Oc3cc(-c4ccc5c(c4)C(c4ccccc4)(c4ccccc4)c4cc6ccccc6cc4-5)ccc32)cc1 ; CC1(c2ccccc2)c2cc(-c3ccc4c(c3)Oc3ccccc3C4(c3ccccc3)c3ccccc3)ccc2-c2cc3ccccc3cc21 ; CC1(C)c2ccccc2Oc2cccc(-c3ccccc3C3(c4ccccc4)c4ccccc4-c4ccc5ccccc5c43)c21 ; CC1(C)c2ccccc2Oc2cccc(-c3ccccc3C3(c4ccccc4)c4ccccc4-c4ccccc43)c21 ; CC1(C)c2ccccc2Oc2cccc(-c3cccc(C4(c5ccccc5)c5ccccc5-c5c4ccc4ccccc54)c3)c21]

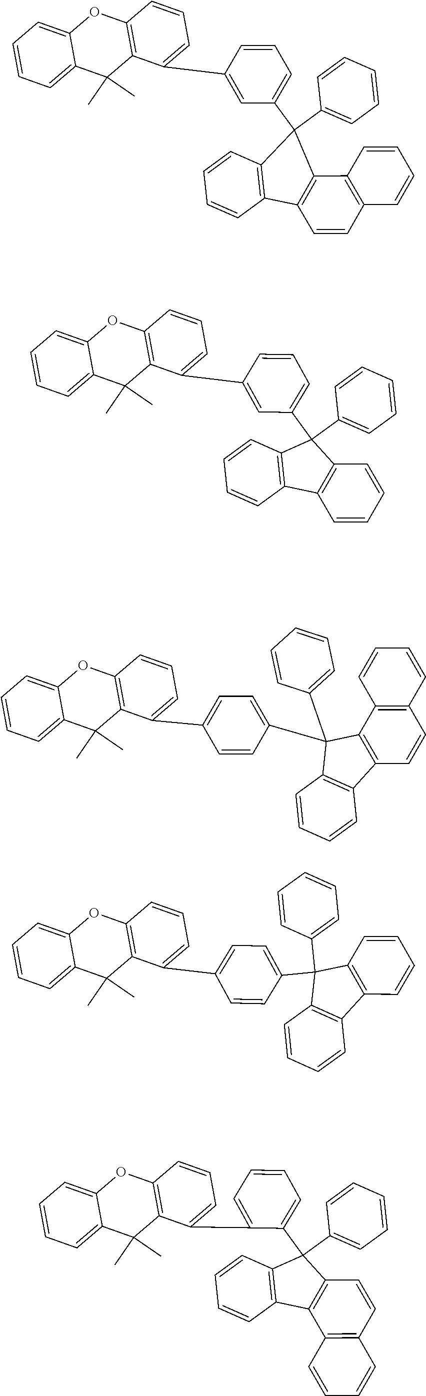

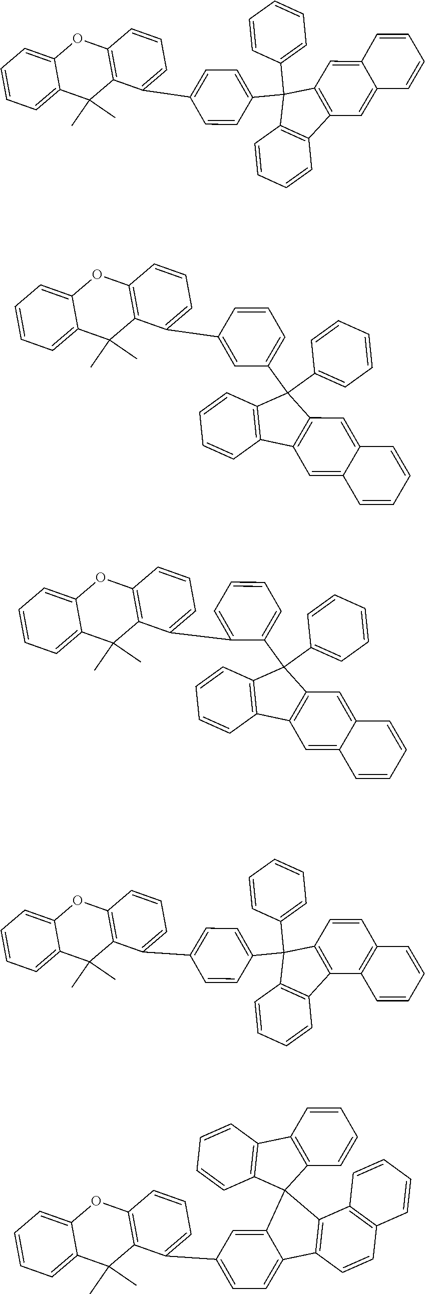 [SMILES: CC1(C)c2ccccc2Oc2cccc(-c3ccc(C4(c5ccccc5)c5ccccc5-c5cc6ccccc6cc54)cc3)c21 ; CC1(C)c2ccccc2Oc2cccc(-c3cccc(C4(c5ccccc5)c5ccccc5-c5cc6ccccc6cc54)c3)c21 ; CC1(C)c2ccccc2Oc2cccc(-c3ccccc3C3(c4ccccc4)c4ccccc4-c4cc5ccccc5cc43)c21 ; CC1(C)c2ccccc2Oc2cccc(-c3ccc(C4(c5ccccc5)c5ccccc5-c5c4ccc4ccccc54)cc3)c21 ; CC1(C)c2ccccc2Oc2cccc(-c3ccc4c(c3)C3(c5ccccc5-c5ccccc53)c3c-4ccc4ccccc34)c21]

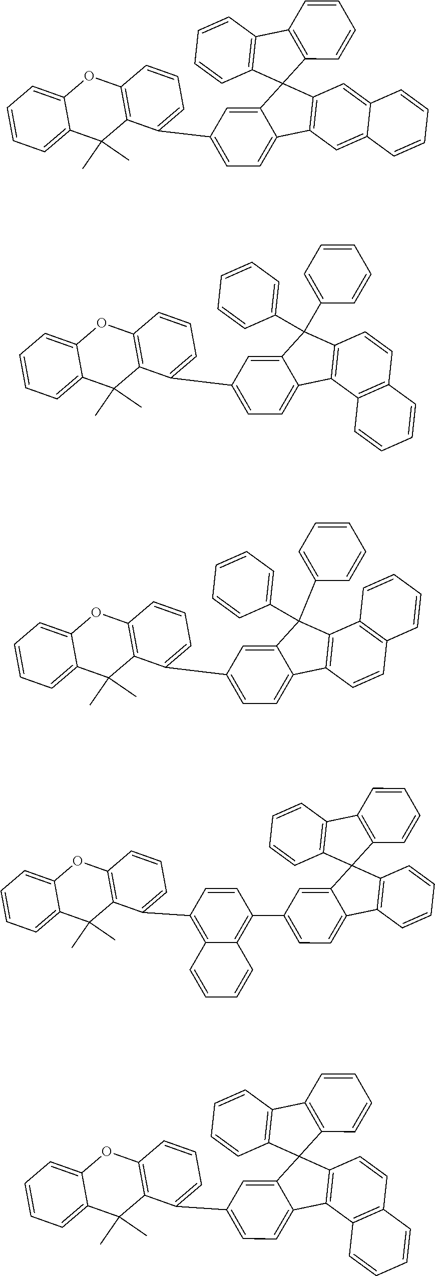 [SMILES: CC1(C)c2ccccc2Oc2cccc(-c3ccc4c(c3)C3(c5ccccc5-c5ccccc53)c3cc5ccccc5cc3-4)c21 ; CC1(C)c2ccccc2Oc2cccc(-c3ccc4c(c3)C(c3ccccc3)(c3ccccc3)c3ccc5ccccc5c3-4)c21 ; CC1(C)c2ccccc2Oc2cccc(-c3ccc4c(c3)C(c3ccccc3)(c3ccccc3)c3c-4ccc4ccccc34)c21 ; CC1(C)c2ccccc2Oc2cccc(-c3ccc(-c4ccc5c(c4)C4(c6ccccc6-c6ccccc64)c4ccccc4-5)c4ccccc34)c21 ; CC1(C)c2ccccc2Oc2cccc(-c3ccc4c(c3)C3(c5ccccc5-c5ccccc53)c3ccc5ccccc5c3-4)c21]

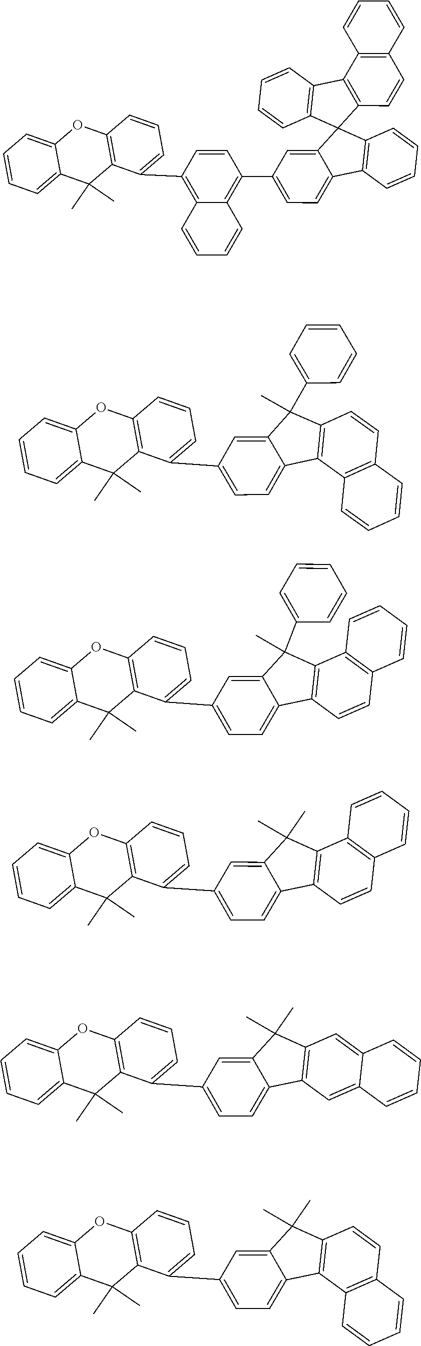 [SMILES: CC1(C)c2ccccc2Oc2cccc(-c3ccc(-c4ccc5c(c4)C4(c6ccccc6-5)c5ccccc5-c5c4ccc4ccccc54)c4ccccc34)c21 ; CC1(C)c2ccccc2Oc2cccc(-c3ccc4c(c3)C(C)(c3ccccc3)c3ccc5ccccc5c3-4)c21 ; CC1(C)c2ccccc2Oc2cccc(-c3ccc4c(c3)C(C)(c3ccccc3)c3c-4ccc4ccccc34)c21 ; CC1(C)c2ccccc2Oc2cccc(-c3ccc4c(c3)C(C)(C)c3c-4ccc4ccccc34)c21 ; CC1(C)c2cc(-c3cccc4c3C(C)(C)c3ccccc3O4)ccc2-c2cc3ccccc3cc21 ; CC1(C)c2cc(-c3cccc4c3C(C)(C)c3ccccc3O4)ccc2-c2c1ccc1ccccc21]

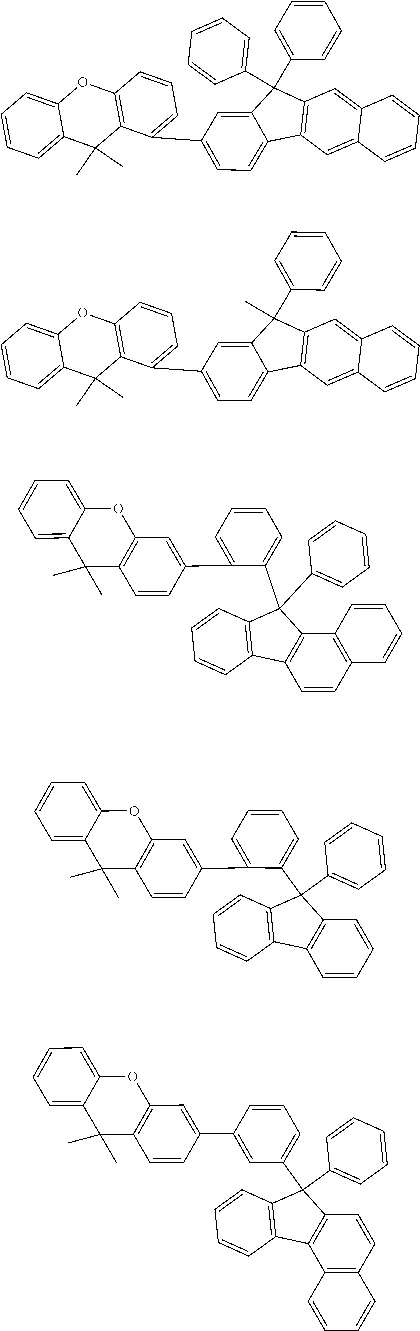 [SMILES: CC1(C)c2ccccc2Oc2cccc(-c3ccc4c(c3)C(c3ccccc3)(c3ccccc3)c3cc5ccccc5cc3-4)c21 ; CC1(C)c2ccccc2Oc2cccc(-c3ccc4c(c3)C(C)(c3ccccc3)c3cc5ccccc5cc3-4)c21 ; CC1(C)c2ccccc2Oc2cc(-c3ccccc3C3(c4ccccc4)c4ccccc4-c4ccc5ccccc5c43)ccc21 ; CC1(C)c2ccccc2Oc2cc(-c3ccccc3C3(c4ccccc4)c4ccccc4-c4ccccc43)ccc21 ; CC1(C)c2ccccc2Oc2cc(-c3cccc(C4(c5ccccc5)c5ccccc5-c5c4ccc4ccccc54)c3)ccc21]

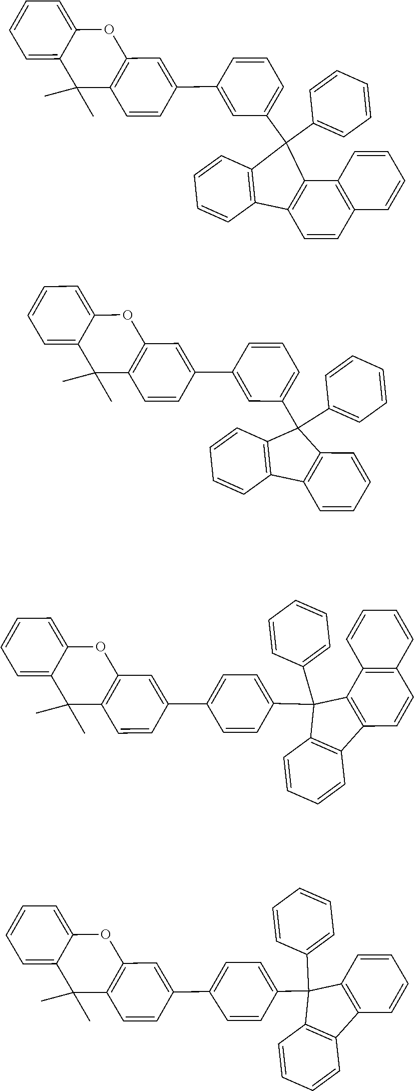 [SMILES: CC1(C)c2ccccc2Oc2cc(-c3cccc(C4(c5ccccc5)c5ccccc5-c5ccc6ccccc6c54)c3)ccc21 ; CC1(C)c2ccccc2Oc2cc(-c3cccc(C4(c5ccccc5)c5ccccc5-c5ccccc54)c3)ccc21 ; CC1(C)c2ccccc2Oc2cc(-c3ccc(C4(c5ccccc5)c5ccccc5-c5ccc6ccccc6c54)cc3)ccc21 ; CC1(C)c2ccccc2Oc2cc(-c3ccc(C4(c5ccccc5)c5ccccc5-c5ccccc54)cc3)ccc21]

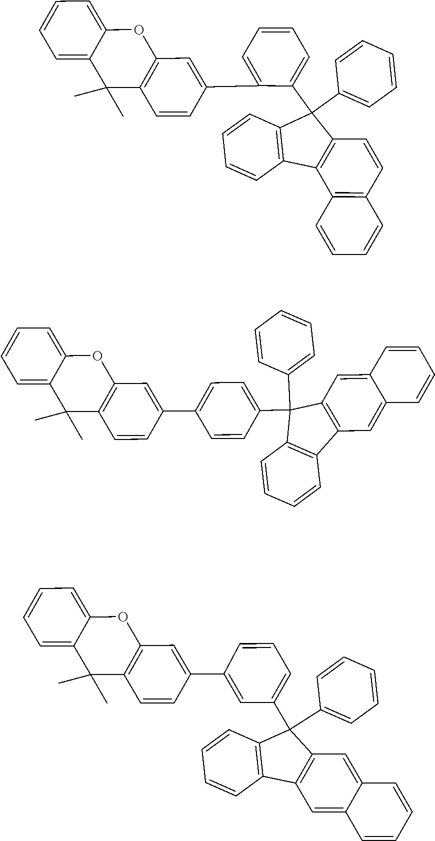 [SMILES: CC1(C)c2ccccc2Oc2cc(-c3ccccc3C3(c4ccccc4)c4ccccc4-c4c3ccc3ccccc43)ccc21 ; CC1(C)c2ccccc2Oc2cc(-c3ccc(C4(c5ccccc5)c5ccccc5-c5cc6ccccc6cc54)cc3)ccc21 ; CC1(C)c2ccccc2Oc2cc(-c3cccc(C4(c5ccccc5)c5ccccc5-c5cc6ccccc6cc54)c3)ccc21]

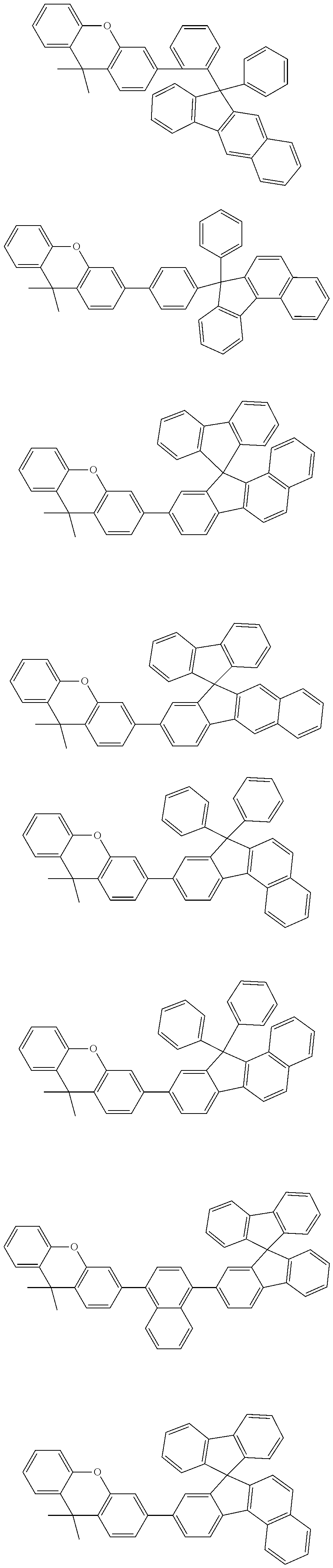

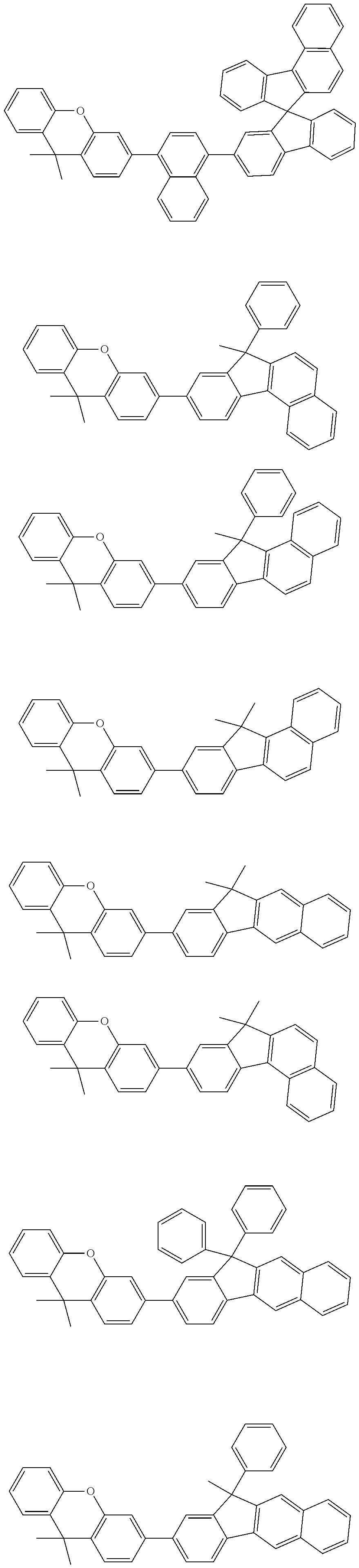

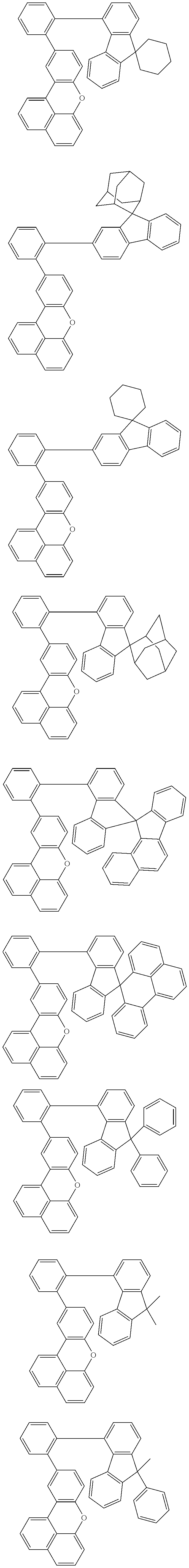

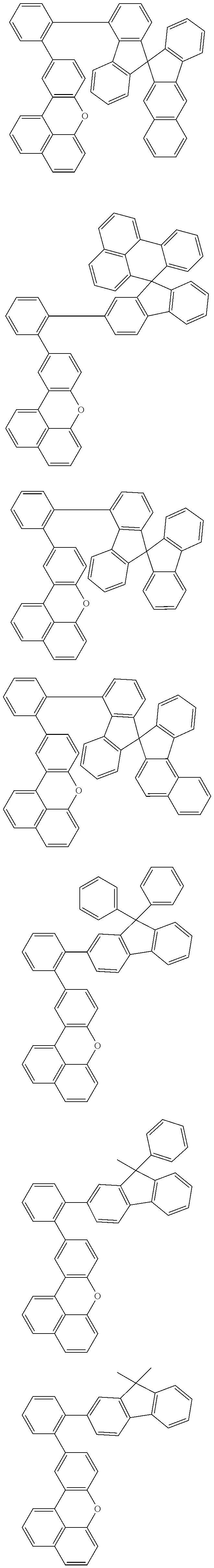

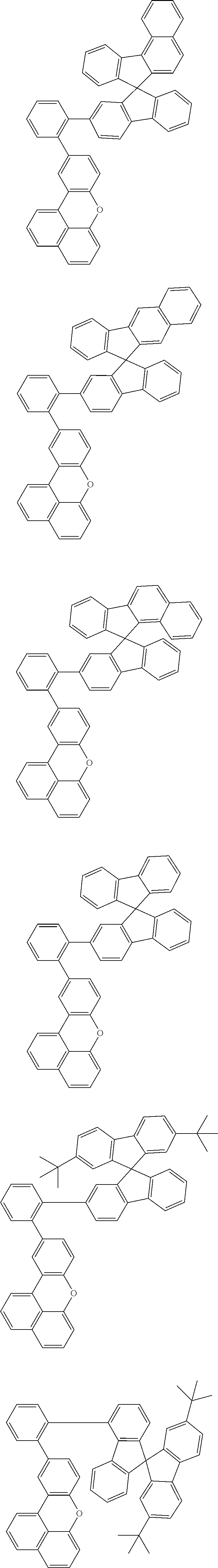

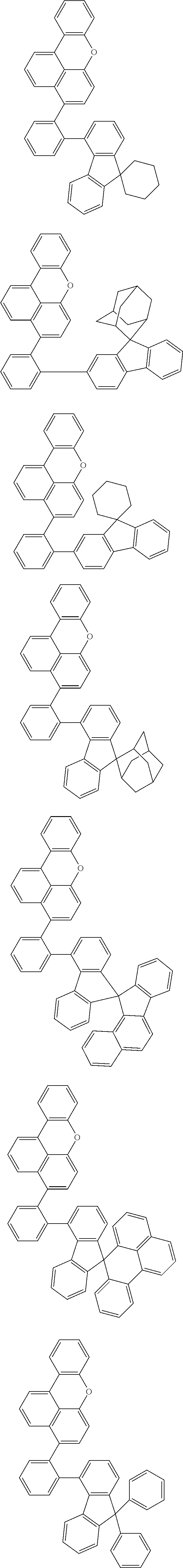

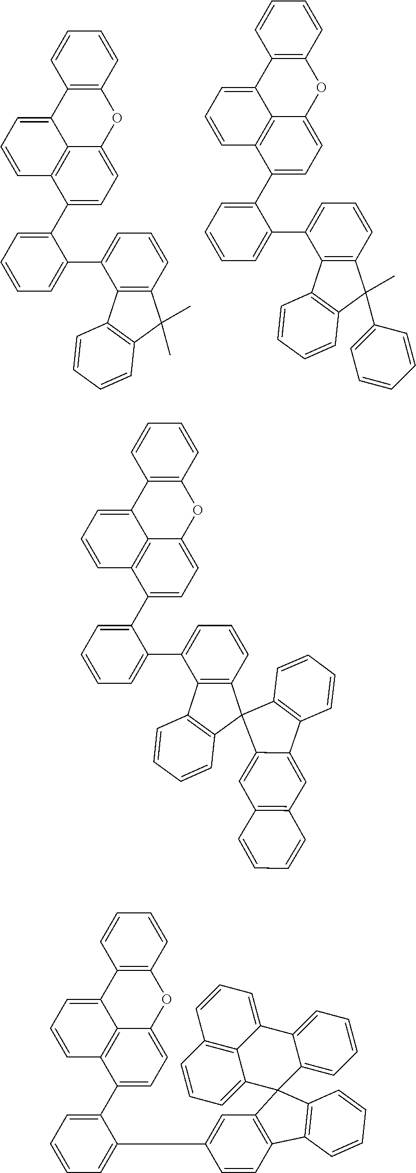 [SMILES: CC1(C)c2ccccc2-c2c(-c3ccccc3-c3ccc4c5c(cccc35)-c3ccccc3O4)cccc21 ; CC1(c2ccccc2)c2ccccc2-c2c(-c3ccccc3-c3ccc4c5c(cccc35)-c3ccccc3O4)cccc21 ; c1ccc2c(c1)Oc1ccc(-c3ccccc3-c3cccc4c3-c3ccccc3C43c4ccccc4-c4cc5ccccc5cc43)c3cccc-2c13 ; c1ccc2c(c1)Oc1ccc(-c3ccccc3-c3ccc4c(c3)C3(c5ccccc5-4)c4ccccc4-c4cccc5cccc3c45)c3cccc-2c13]

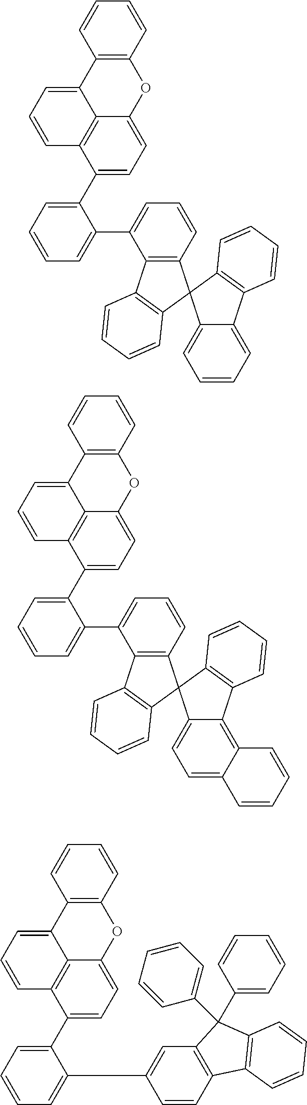

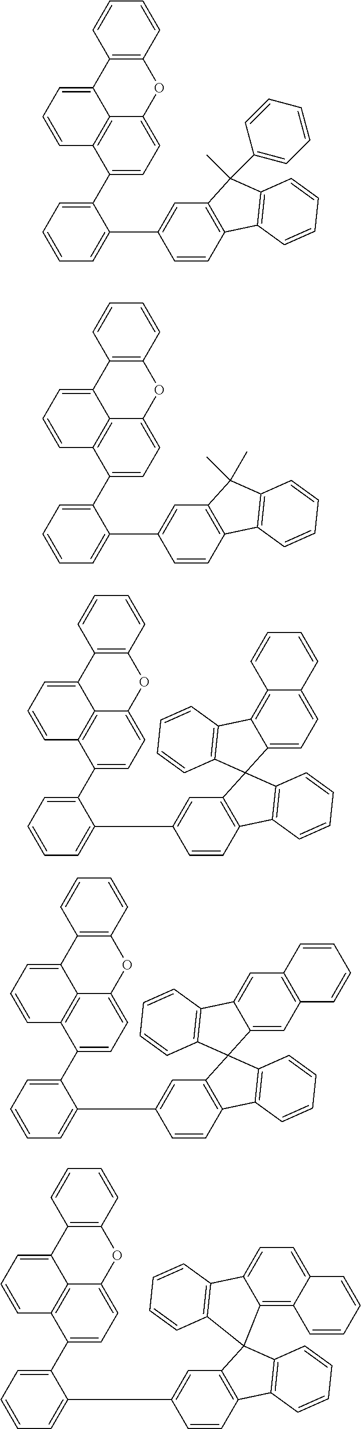

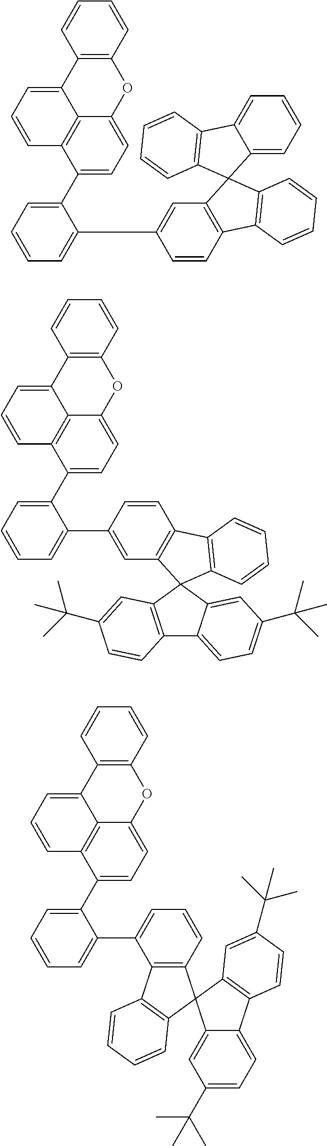

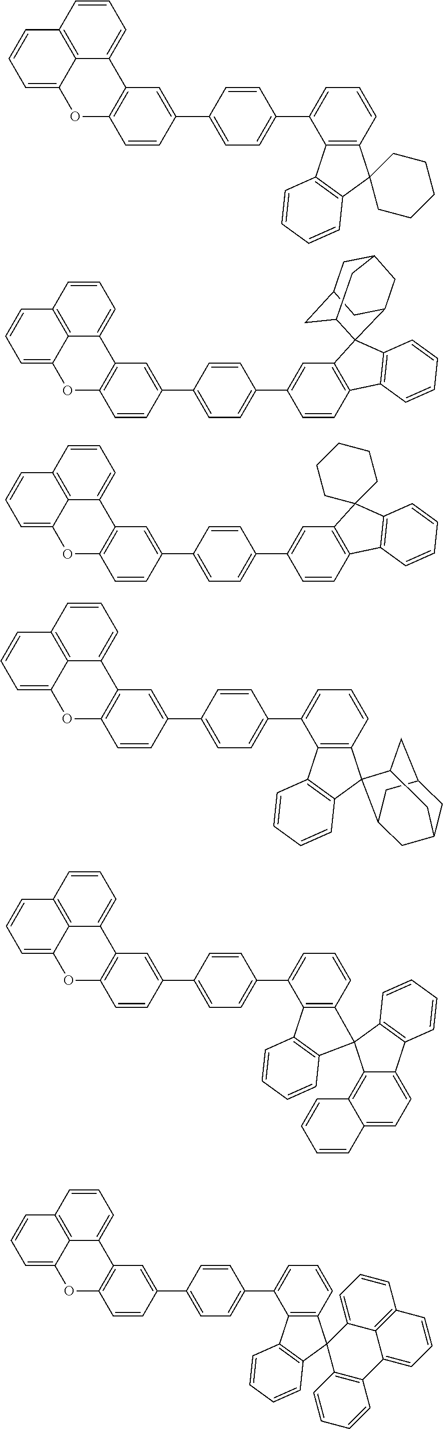

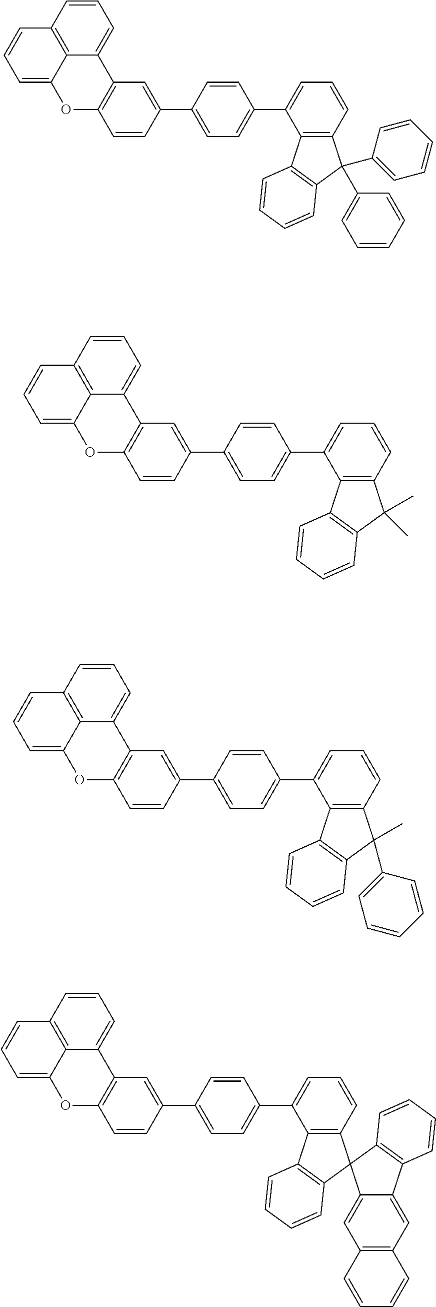 [SMILES: c1ccc(C2(c3ccccc3)c3ccccc3-c3c(-c4ccc(-c5ccc6c(c5)-c5cccc7cccc(c57)O6)cc4)cccc32)cc1 ; CC1(C)c2ccccc2-c2c(-c3ccc(-c4ccc5c(c4)-c4cccc6cccc(c46)O5)cc3)cccc21 ; CC1(c2ccccc2)c2ccccc2-c2c(-c3ccc(-c4ccc5c(c4)-c4cccc6cccc(c46)O5)cc3)cccc21 ; c1ccc2c(c1)-c1cc3ccccc3cc1C21c2ccccc2-c2c(-c3ccc(-c4ccc5c(c4)-c4cccc6cccc(c46)O5)cc3)cccc21]

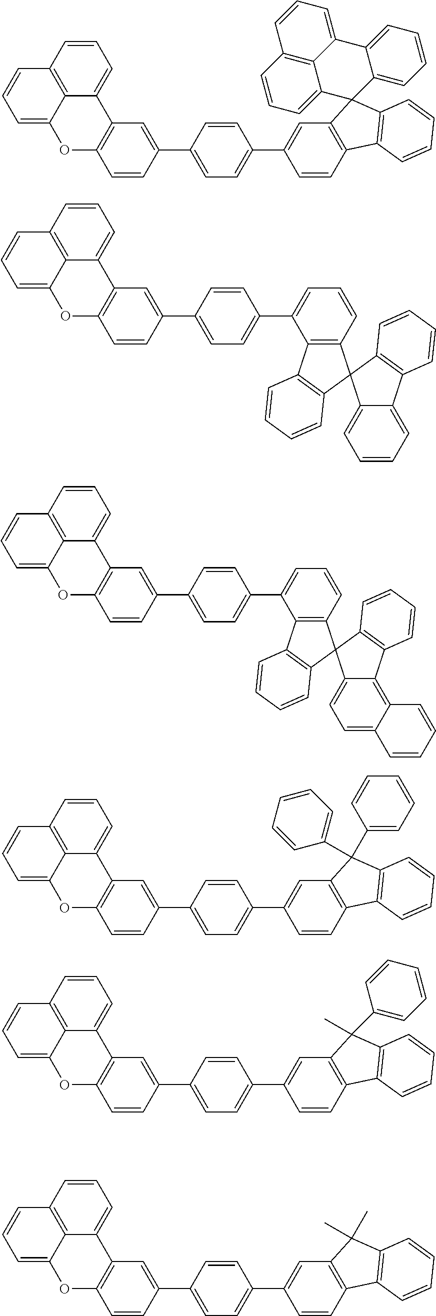

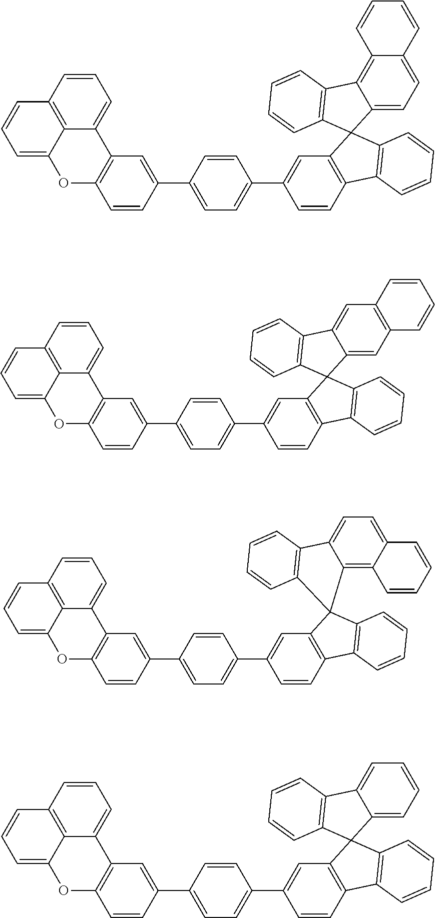 [SMILES: c1ccc2c(c1)-c1ccc(-c3ccc(-c4ccc5c(c4)-c4cccc6cccc(c46)O5)cc3)cc1C21c2ccccc2-c2c1ccc1ccccc21 ; c1ccc2c(c1)-c1ccc(-c3ccc(-c4ccc5c(c4)-c4cccc6cccc(c46)O5)cc3)cc1C21c2ccccc2-c2cc3ccccc3cc21 ; c1ccc2c(c1)-c1ccc(-c3ccc(-c4ccc5c(c4)-c4cccc6cccc(c46)O5)cc3)cc1C21c2ccccc2-c2ccc3ccccc3c21 ; c1ccc2c(c1)-c1ccccc1C21c2ccccc2-c2ccc(-c3ccc(-c4ccc5c(c4)-c4cccc6cccc(c46)O5)cc3)cc21]

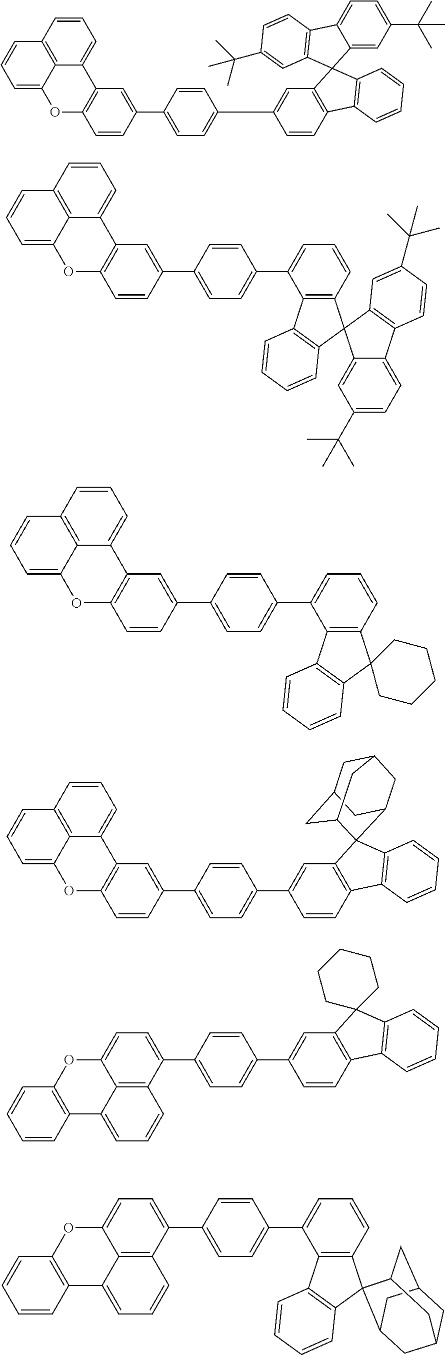

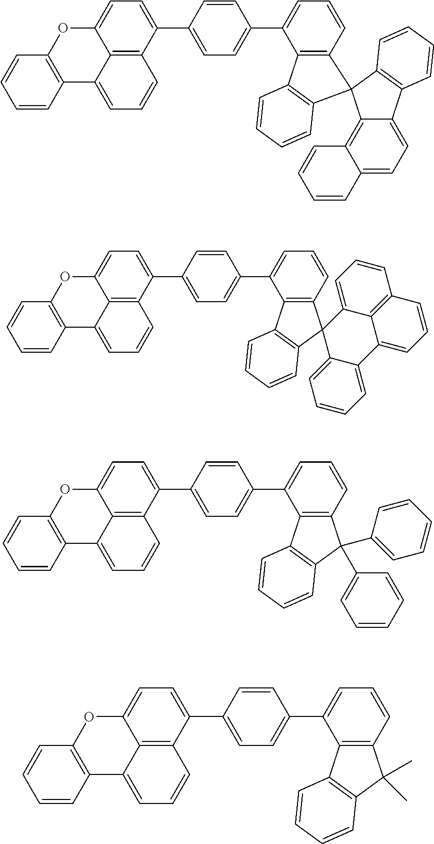 [SMILES: c1ccc2c(c1)Oc1ccc(-c3ccc(-c4cccc5c4-c4ccccc4C54c5ccccc5-c5ccc6ccccc6c54)cc3)c3cccc-2c13 ; c1ccc2c(c1)Oc1ccc(-c3ccc(-c4cccc5c4-c4ccccc4C54c5ccccc5-c5cccc6cccc4c56)cc3)c3cccc-2c13 ; c1ccc(C2(c3ccccc3)c3ccccc3-c3c(-c4ccc(-c5ccc6c7c(cccc57)-c5ccccc5O6)cc4)cccc32)cc1 ; CC1(C)c2ccccc2-c2c(-c3ccc(-c4ccc5c6c(cccc46)-c4ccccc4O5)cc3)cccc21]

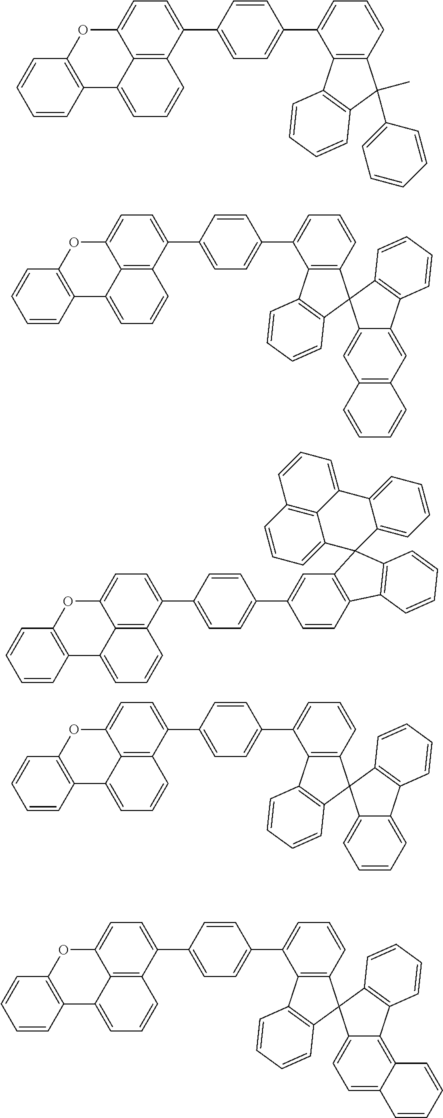

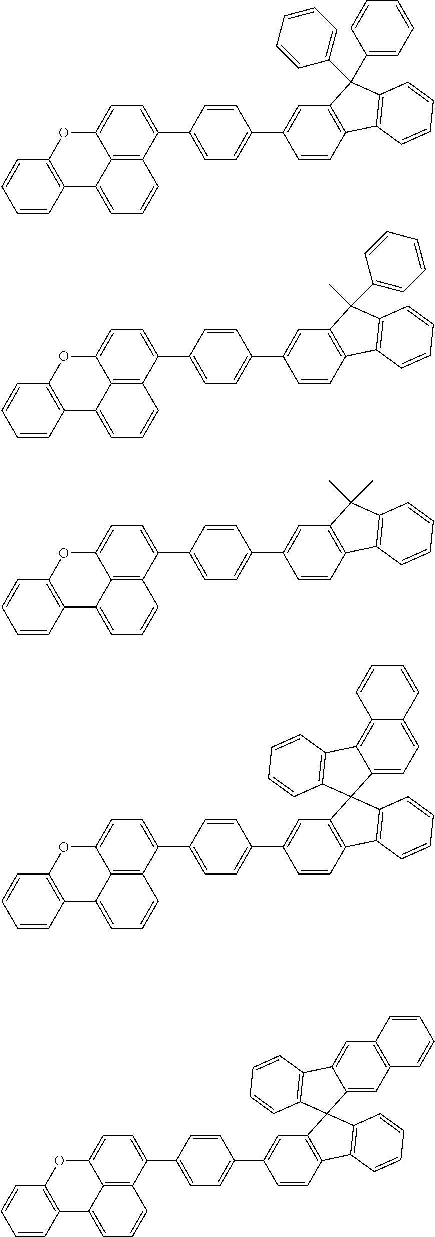 [SMILES: c1ccc(C2(c3ccccc3)c3ccccc3-c3ccc(-c4ccc(-c5ccc6c7c(cccc57)-c5ccccc5O6)cc4)cc32)cc1 ; CC1(c2ccccc2)c2ccccc2-c2ccc(-c3ccc(-c4ccc5c6c(cccc46)-c4ccccc4O5)cc3)cc21 ; CC1(C)c2ccccc2-c2ccc(-c3ccc(-c4ccc5c6c(cccc46)-c4ccccc4O5)cc3)cc21 ; c1ccc2c(c1)Oc1ccc(-c3ccc(-c4ccc5c(c4)C4(c6ccccc6-5)c5ccccc5-c5c4ccc4ccccc54)cc3)c3cccc-2c13 ; c1ccc2c(c1)Oc1ccc(-c3ccc(-c4ccc5c(c4)C4(c6ccccc6-5)c5ccccc5-c5cc6ccccc6cc54)cc3)c3cccc-2c13]

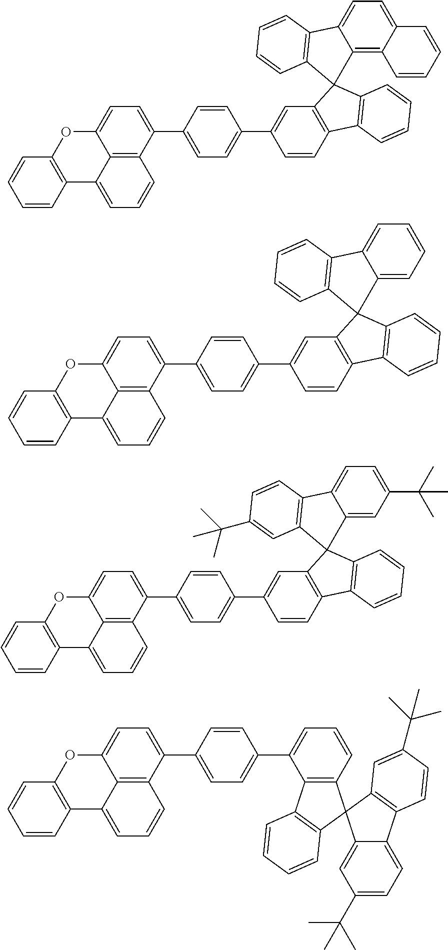 [SMILES: c1ccc2c(c1)Oc1ccc(-c3ccc(-c4ccc5c(c4)C4(c6ccccc6-5)c5ccccc5-c5ccc6ccccc6c54)cc3)c3cccc-2c13 ; c1ccc2c(c1)Oc1ccc(-c3ccc(-c4ccc5c(c4)C4(c6ccccc6-c6ccccc64)c4ccccc4-5)cc3)c3cccc-2c13 ; CC(C)(C)c1ccc2c(c1)C1(c3ccccc3-c3ccc(-c4ccc(-c5ccc6c7c(cccc57)-c5ccccc5O6)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4ccc5c6c(cccc46)-c4ccccc4O5)cc3)cccc21]

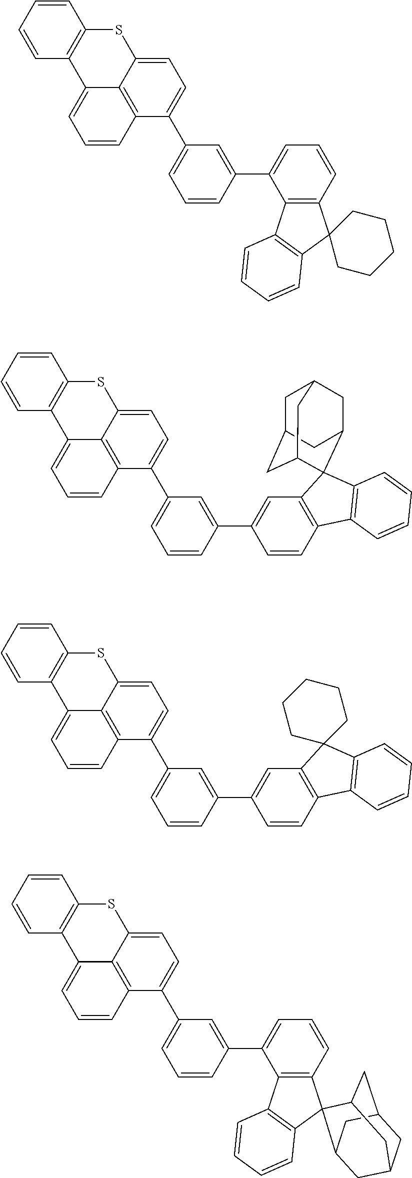 [SMILES: c1cc(-c2cccc3c2-c2ccccc2C32CCCCC2)cc(-c2ccc3c4c(cccc24)-c2ccccc2S3)c1 ; c1cc(-c2ccc3c(c2)C2(c4ccccc4-3)C3CC4CC(C3)CC2C4)cc(-c2ccc3c4c(cccc24)-c2ccccc2S3)c1 ; c1cc(-c2ccc3c(c2)C2(CCCCC2)c2ccccc2-3)cc(-c2ccc3c4c(cccc24)-c2ccccc2S3)c1 ; c1cc(-c2cccc3c2-c2ccccc2C32C3CC4CC(C3)CC2C4)cc(-c2ccc3c4c(cccc24)-c2ccccc2S3)c1]

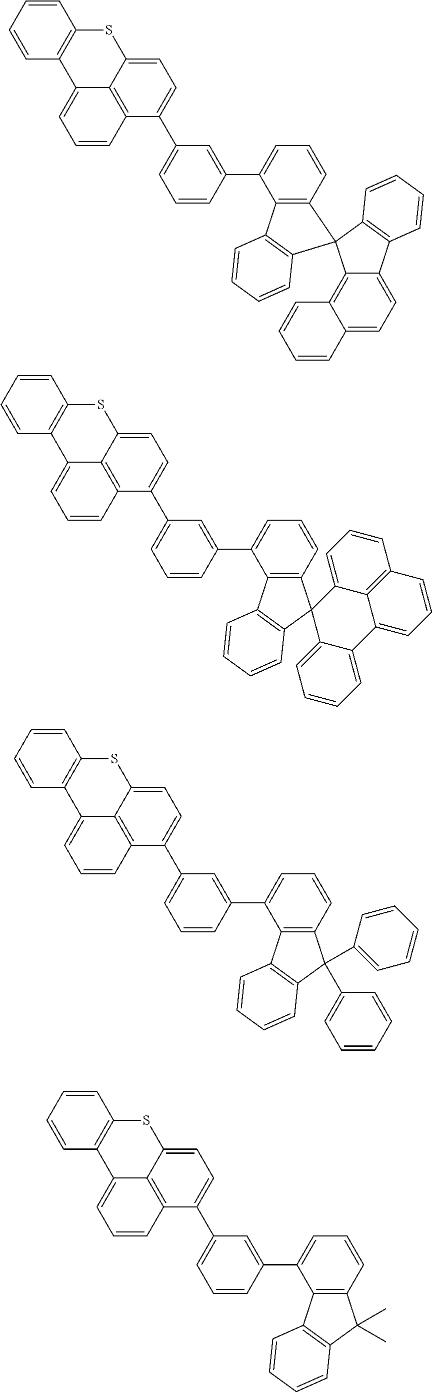 [SMILES: c1cc(-c2cccc3c2-c2ccccc2C32c3ccccc3-c3ccc4ccccc4c32)cc(-c2ccc3c4c(cccc24)-c2ccccc2S3)c1 ; c1cc(-c2cccc3c2-c2ccccc2C32c3ccccc3-c3cccc4cccc2c34)cc(-c2ccc3c4c(cccc24)-c2ccccc2S3)c1 ; c1ccc(C2(c3ccccc3)c3ccccc3-c3c(-c4cccc(-c5ccc6c7c(cccc57)-c5ccccc5S6)c4)cccc32)cc1 ; CC1(C)c2ccccc2-c2c(-c3cccc(-c4ccc5c6c(cccc46)-c4ccccc4S5)c3)cccc21]

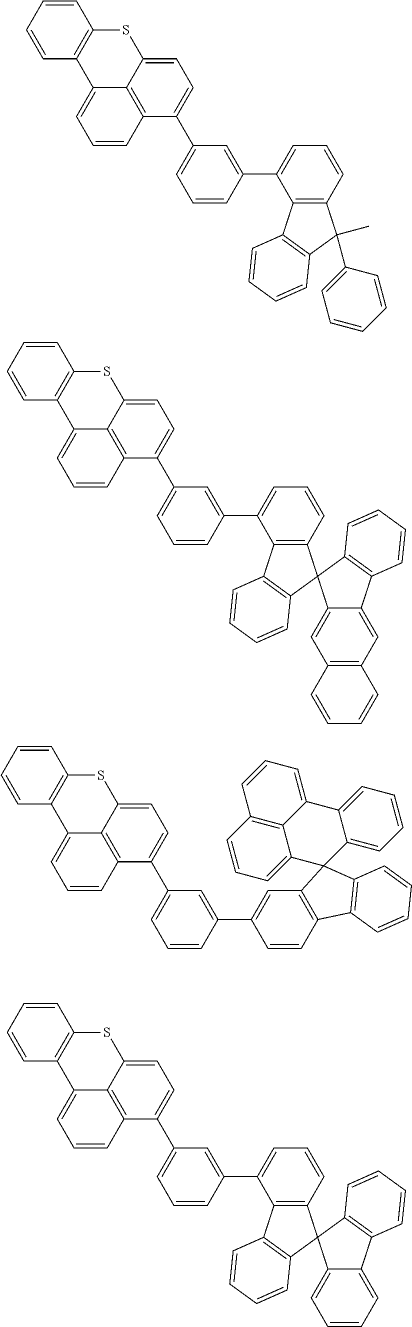 [SMILES: CC1(c2ccccc2)c2ccccc2-c2c(-c3cccc(-c4ccc5c6c(cccc46)-c4ccccc4S5)c3)cccc21 ; c1cc(-c2cccc3c2-c2ccccc2C32c3ccccc3-c3cc4ccccc4cc32)cc(-c2ccc3c4c(cccc24)-c2ccccc2S3)c1 ; c1cc(-c2ccc3c(c2)C2(c4ccccc4-3)c3ccccc3-c3cccc4cccc2c34)cc(-c2ccc3c4c(cccc24)-c2ccccc2S3)c1 ; c1cc(-c2cccc3c2-c2ccccc2C32c3ccccc3-c3ccccc32)cc(-c2ccc3c4c(cccc24)-c2ccccc2S3)c1]

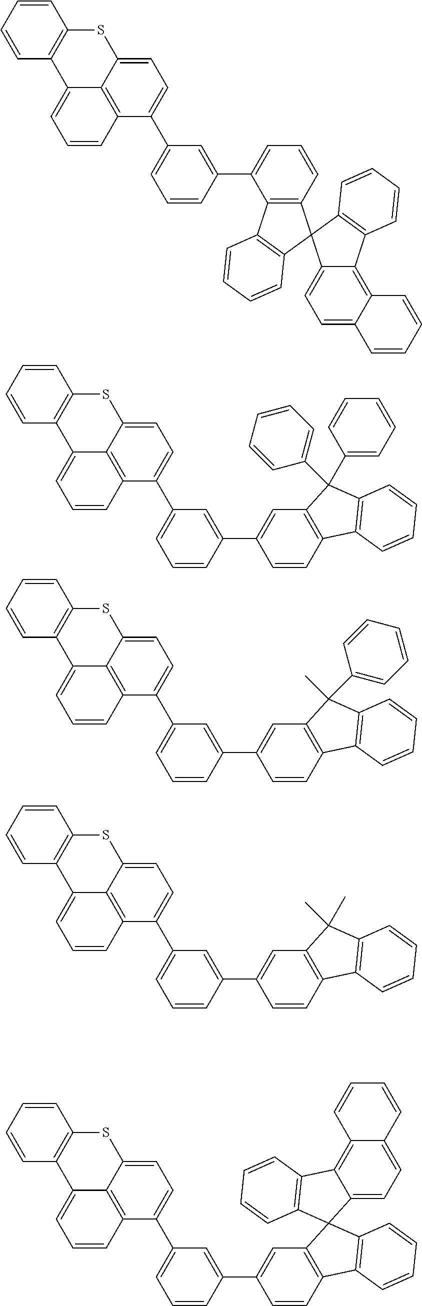

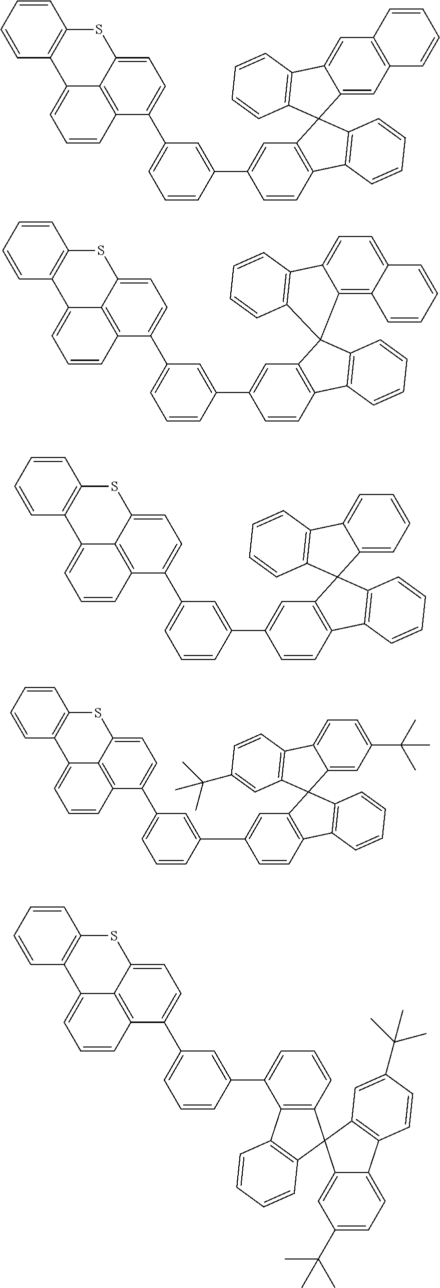 [SMILES: c1cc(-c2ccc3c(c2)C2(c4ccccc4-3)c3ccccc3-c3cc4ccccc4cc32)cc(-c2ccc3c4c(cccc24)-c2ccccc2S3)c1 ; c1cc(-c2ccc3c(c2)C2(c4ccccc4-3)c3ccccc3-c3ccc4ccccc4c32)cc(-c2ccc3c4c(cccc24)-c2ccccc2S3)c1 ; c1cc(-c2ccc3c(c2)C2(c4ccccc4-c4ccccc42)c2ccccc2-3)cc(-c2ccc3c4c(cccc24)-c2ccccc2S3)c1 ; CC(C)(C)c1ccc2c(c1)C1(c3ccccc3-c3ccc(-c4cccc(-c5ccc6c7c(cccc57)-c5ccccc5S6)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4ccc5c6c(cccc46)-c4ccccc4S5)c3)cccc21]

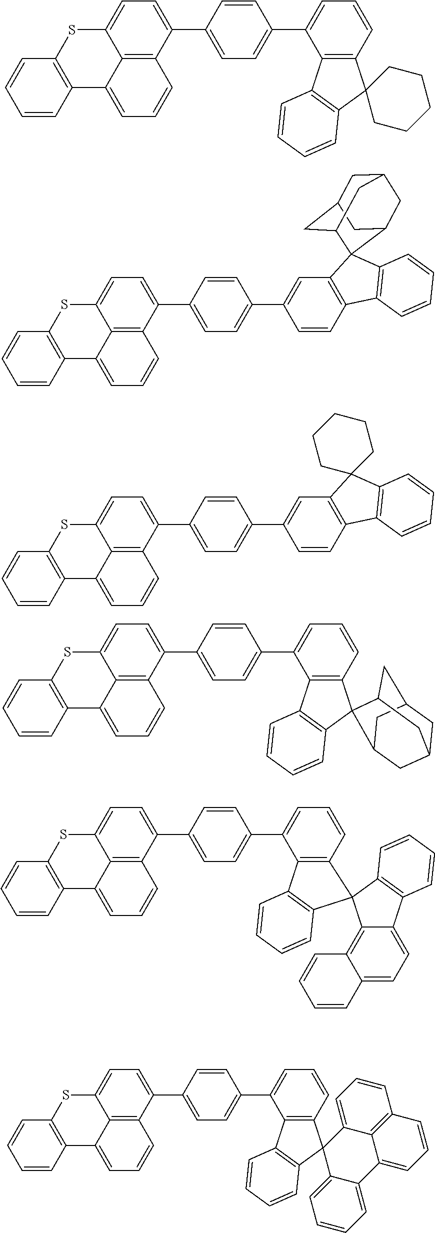

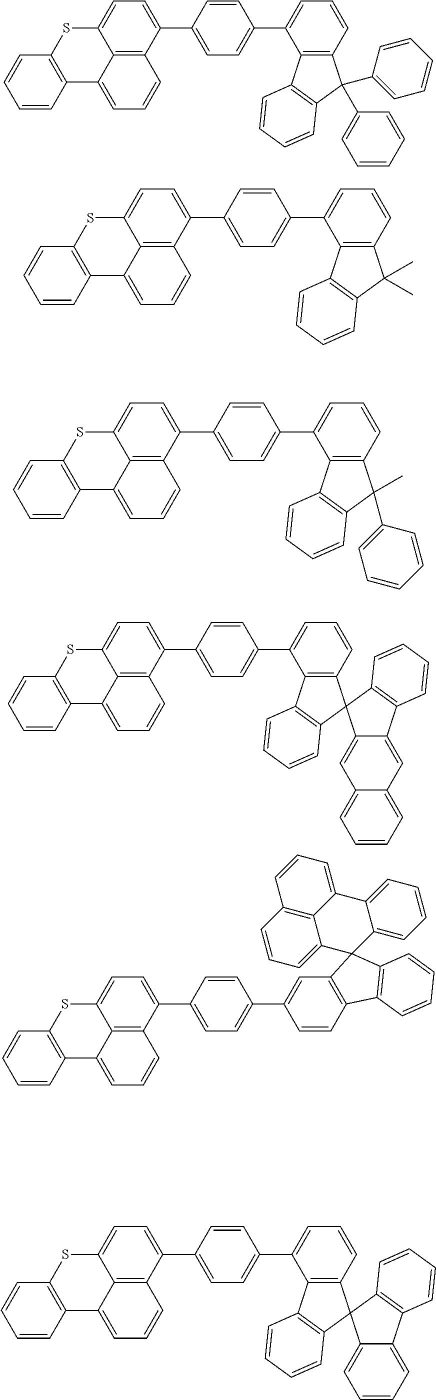

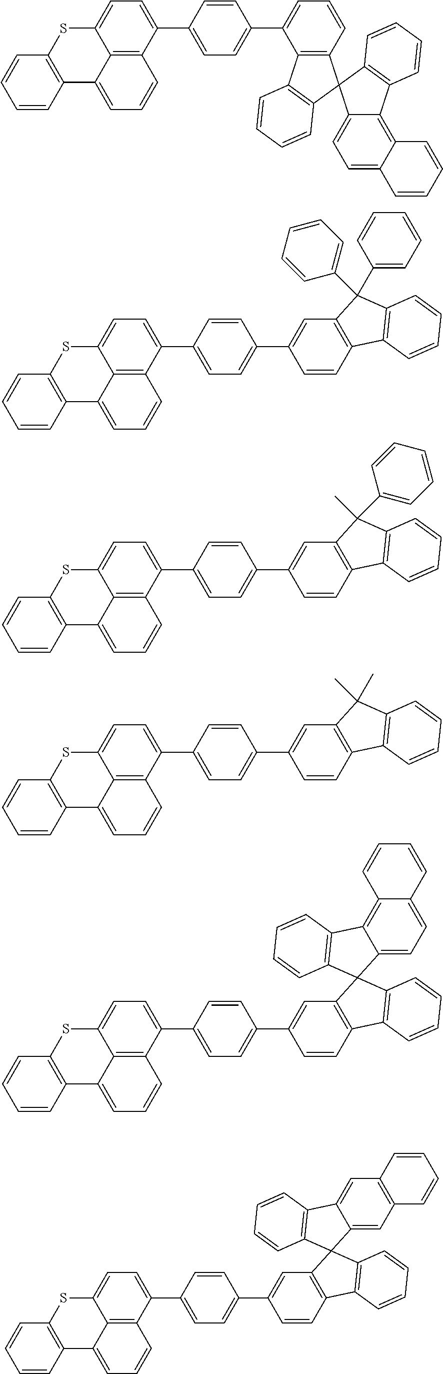

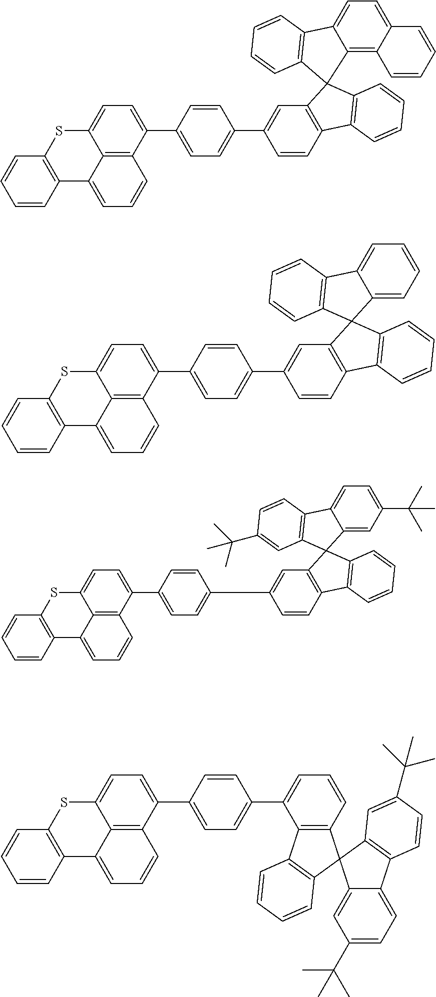 [SMILES: c1ccc2c(c1)Sc1ccc(-c3ccc(-c4ccc5c(c4)C4(c6ccccc6-5)c5ccccc5-c5ccc6ccccc6c54)cc3)c3cccc-2c13 ; c1ccc2c(c1)Sc1ccc(-c3ccc(-c4ccc5c(c4)C4(c6ccccc6-c6ccccc64)c4ccccc4-5)cc3)c3cccc-2c13 ; CC(C)(C)c1ccc2c(c1)C1(c3ccccc3-c3ccc(-c4ccc(-c5ccc6c7c(cccc57)-c5ccccc5S6)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4ccc5c6c(cccc46)-c4ccccc4S5)cc3)cccc21]

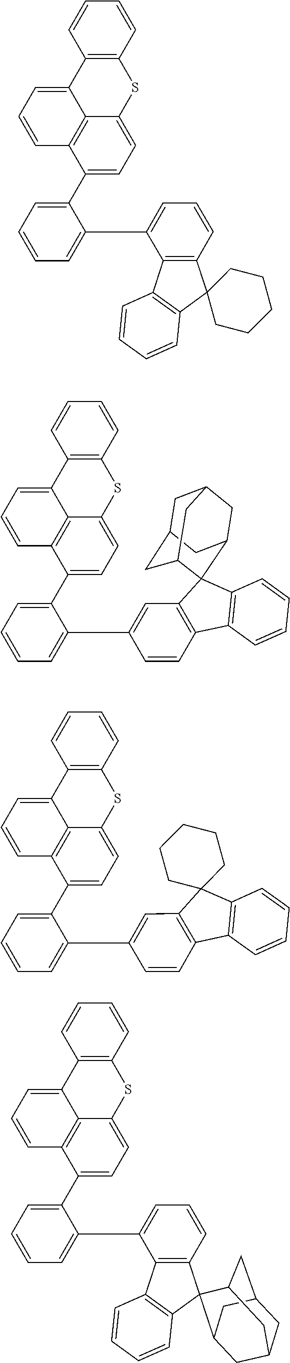

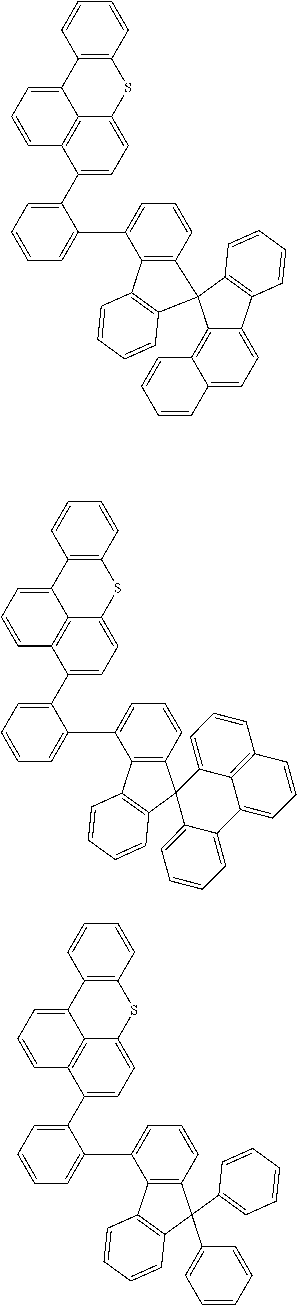 [SMILES: c1ccc2c(c1)Sc1ccc(-c3ccccc3-c3cccc4c3-c3ccccc3C43c4ccccc4-c4ccc5ccccc5c43)c3cccc-2c13 ; c1ccc2c(c1)Sc1ccc(-c3ccccc3-c3cccc4c3-c3ccccc3C43c4ccccc4-c4cccc5cccc3c45)c3cccc-2c13 ; c1ccc(C2(c3ccccc3)c3ccccc3-c3c(-c4ccccc4-c4ccc5c6c(cccc46)-c4ccccc4S5)cccc32)cc1]

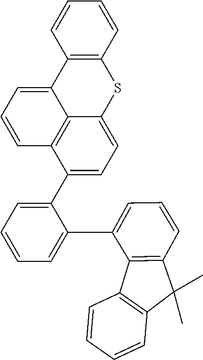 [SMILES: CC1(C)c2ccccc2-c2c(-c3ccccc3-c3ccc4c5c(cccc35)-c3ccccc3S4)cccc21]

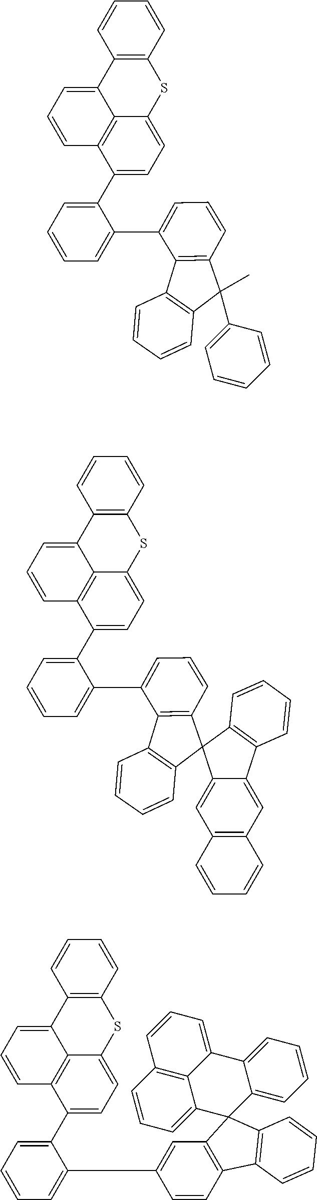 [SMILES: CC1(c2ccccc2)c2ccccc2-c2c(-c3ccccc3-c3ccc4c5c(cccc35)-c3ccccc3S4)cccc21 ; c1ccc2c(c1)Sc1ccc(-c3ccccc3-c3cccc4c3-c3ccccc3C43c4ccccc4-c4cc5ccccc5cc43)c3cccc-2c13 ; c1ccc2c(c1)Sc1ccc(-c3ccccc3-c3ccc4c(c3)C3(c5ccccc5-4)c4ccccc4-c4cccc5cccc3c45)c3cccc-2c13]

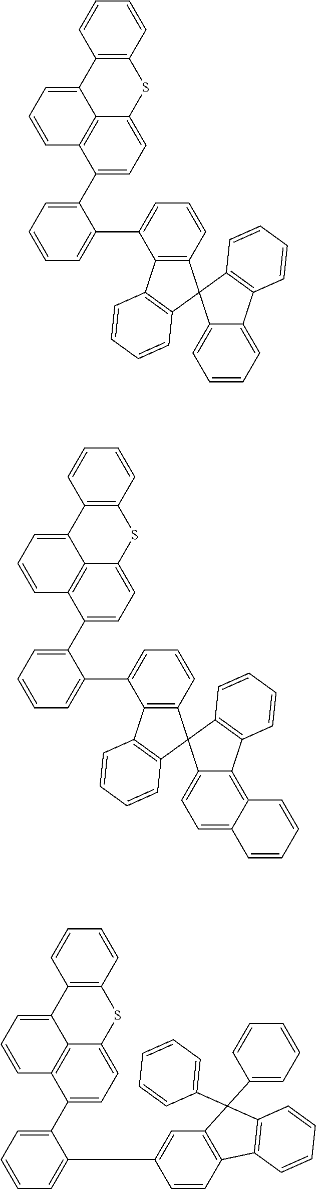 [SMILES: c1ccc2c(c1)Sc1ccc(-c3ccccc3-c3cccc4c3-c3ccccc3C43c4ccccc4-c4ccccc43)c3cccc-2c13 ; c1ccc2c(c1)Sc1ccc(-c3ccccc3-c3cccc4c3-c3ccccc3C43c4ccccc4-c4c3ccc3ccccc43)c3cccc-2c13 ; c1ccc(C2(c3ccccc3)c3ccccc3-c3ccc(-c4ccccc4-c4ccc5c6c(cccc46)-c4ccccc4S5)cc32)cc1]

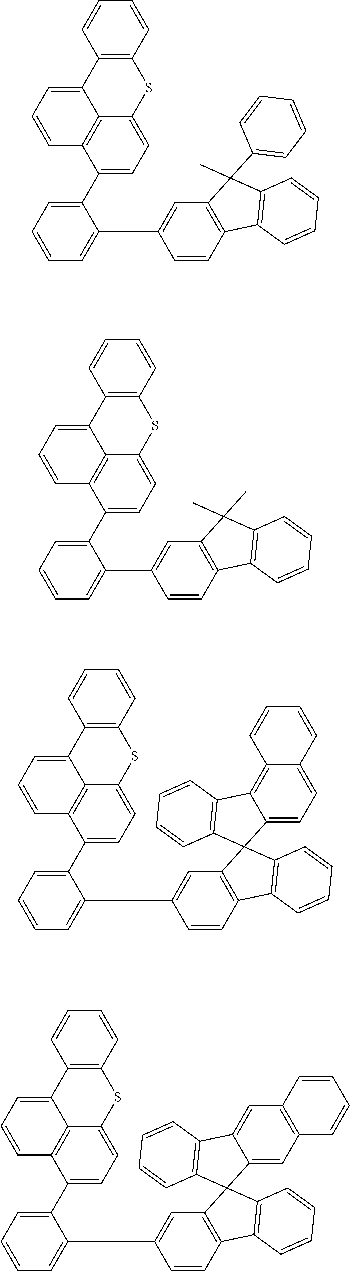 [SMILES: CC1(c2ccccc2)c2ccccc2-c2ccc(-c3ccccc3-c3ccc4c5c(cccc35)-c3ccccc3S4)cc21 ; CC1(C)c2ccccc2-c2ccc(-c3ccccc3-c3ccc4c5c(cccc35)-c3ccccc3S4)cc21 ; c1ccc2c(c1)Sc1ccc(-c3ccccc3-c3ccc4c(c3)C3(c5ccccc5-4)c4ccccc4-c4c3ccc3ccccc43)c3cccc-2c13 ; c1ccc2c(c1)Sc1ccc(-c3ccccc3-c3ccc4c(c3)C3(c5ccccc5-4)c4ccccc4-c4cc5ccccc5cc43)c3cccc-2c13]

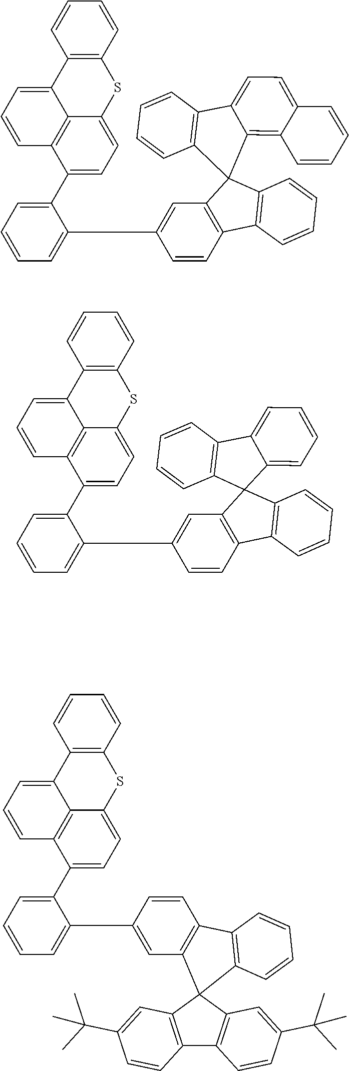 [SMILES: c1ccc2c(c1)Sc1ccc(-c3ccccc3-c3ccc4c(c3)C3(c5ccccc5-4)c4ccccc4-c4ccc5ccccc5c43)c3cccc-2c13 ; c1ccc2c(c1)Sc1ccc(-c3ccccc3-c3ccc4c(c3)C3(c5ccccc5-c5ccccc53)c3ccccc3-4)c3cccc-2c13 ; CC(C)(C)c1ccc2c(c1)C1(c3ccccc3-c3ccc(-c4ccccc4-c4ccc5c6c(cccc46)-c4ccccc4S5)cc31)c1cc(C(C)(C)C)ccc1-2]

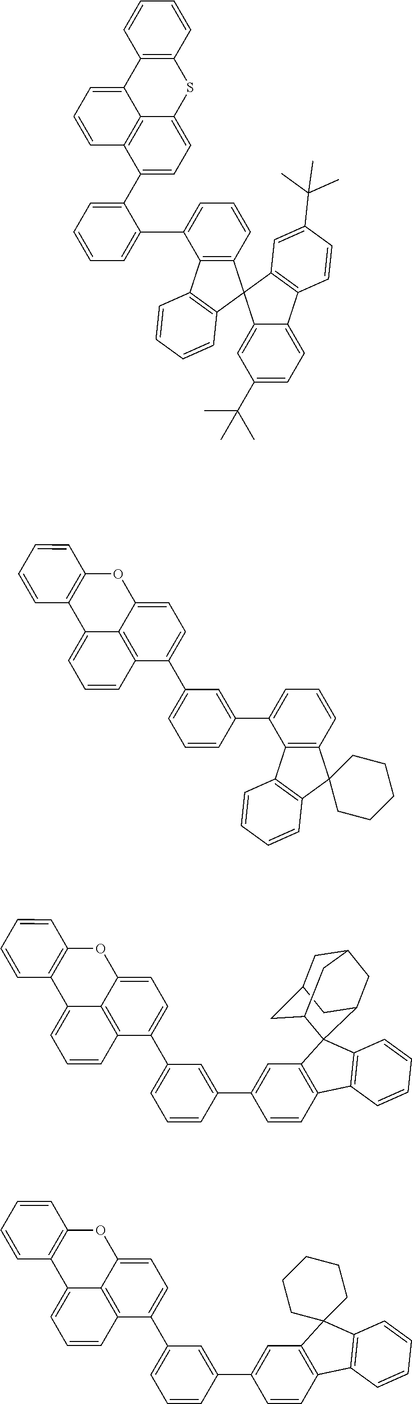 [SMILES: CC(C)(C)c1ccc2c(c1)C1(c3cc(C(C)(C)C)ccc3-2)c2ccccc2-c2c(-c3ccccc3-c3ccc4c5c(cccc35)-c3ccccc3S4)cccc21 ; c1cc(-c2cccc3c2-c2ccccc2C32CCCCC2)cc(-c2ccc3c4c(cccc24)-c2ccccc2O3)c1 ; c1cc(-c2ccc3c(c2)C2(c4ccccc4-3)C3CC4CC(C3)CC2C4)cc(-c2ccc3c4c(cccc24)-c2ccccc2O3)c1 ; c1cc(-c2ccc3c(c2)C2(CCCCC2)c2ccccc2-3)cc(-c2ccc3c4c(cccc24)-c2ccccc2O3)c1]

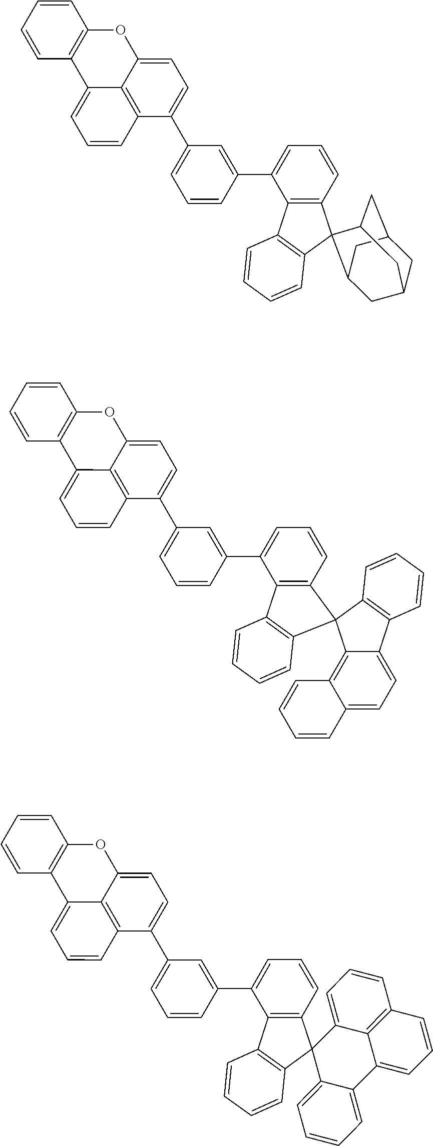 [SMILES: c1cc(-c2cccc3c2-c2ccccc2C32C3CC4CC(C3)CC2C4)cc(-c2ccc3c4c(cccc24)-c2ccccc2O3)c1 ; c1cc(-c2cccc3c2-c2ccccc2C32c3ccccc3-c3ccc4ccccc4c32)cc(-c2ccc3c4c(cccc24)-c2ccccc2O3)c1 ; c1cc(-c2cccc3c2-c2ccccc2C32c3ccccc3-c3cccc4cccc2c34)cc(-c2ccc3c4c(cccc24)-c2ccccc2O3)c1]

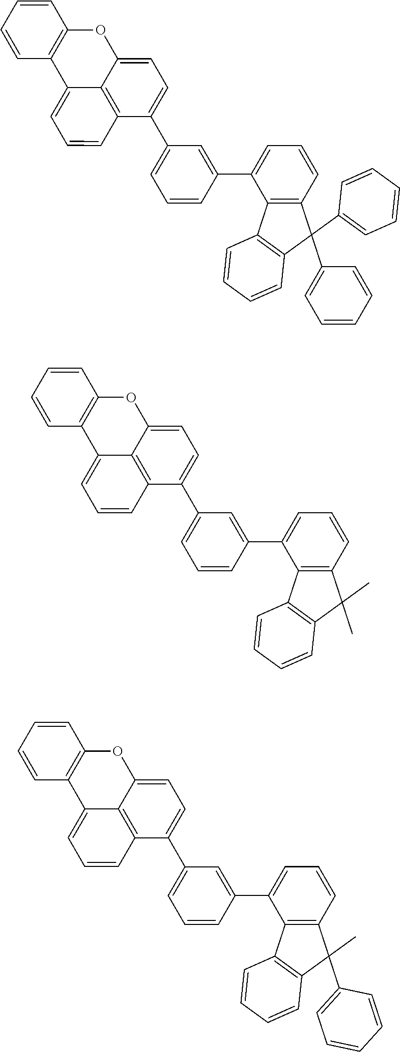 [SMILES: c1ccc(C2(c3ccccc3)c3ccccc3-c3c(-c4cccc(-c5ccc6c7c(cccc57)-c5ccccc5O6)c4)cccc32)cc1 ; CC1(C)c2ccccc2-c2c(-c3cccc(-c4ccc5c6c(cccc46)-c4ccccc4O5)c3)cccc21 ; CC1(c2ccccc2)c2ccccc2-c2c(-c3cccc(-c4ccc5c6c(cccc46)-c4ccccc4O5)c3)cccc21]

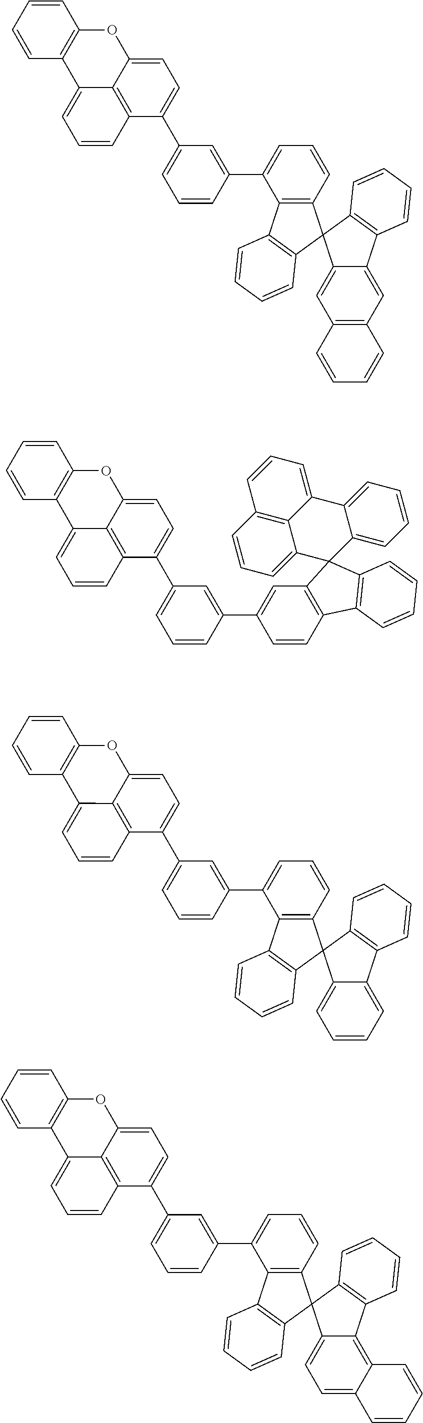 [SMILES: c1cc(-c2cccc3c2-c2ccccc2C32c3ccccc3-c3cc4ccccc4cc32)cc(-c2ccc3c4c(cccc24)-c2ccccc2O3)c1 ; c1cc(-c2ccc3c(c2)C2(c4ccccc4-3)c3ccccc3-c3cccc4cccc2c34)cc(-c2ccc3c4c(cccc24)-c2ccccc2O3)c1 ; c1cc(-c2cccc3c2-c2ccccc2C32c3ccccc3-c3ccccc32)cc(-c2ccc3c4c(cccc24)-c2ccccc2O3)c1 ; c1cc(-c2cccc3c2-c2ccccc2C32c3ccccc3-c3c2ccc2ccccc32)cc(-c2ccc3c4c(cccc24)-c2ccccc2O3)c1]

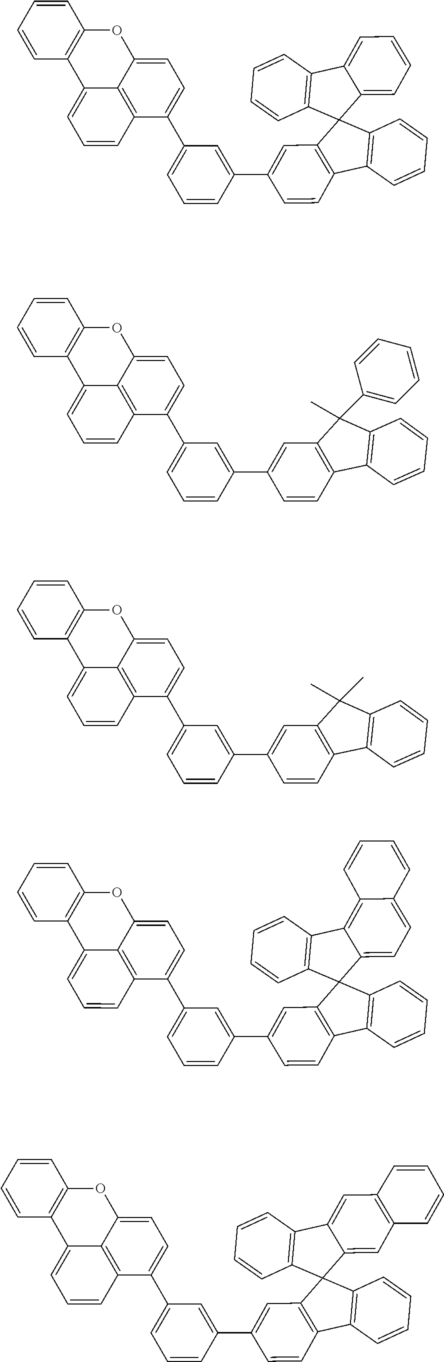 [SMILES: c1cc(-c2ccc3c(c2)C2(c4ccccc4-c4ccccc42)c2ccccc2-3)cc(-c2ccc3c4c(cccc24)-c2ccccc2O3)c1 ; CC1(c2ccccc2)c2ccccc2-c2ccc(-c3cccc(-c4ccc5c6c(cccc46)-c4ccccc4O5)c3)cc21 ; CC1(C)c2ccccc2-c2ccc(-c3cccc(-c4ccc5c6c(cccc46)-c4ccccc4O5)c3)cc21 ; c1cc(-c2ccc3c(c2)C2(c4ccccc4-3)c3ccccc3-c3c2ccc2ccccc32)cc(-c2ccc3c4c(cccc24)-c2ccccc2O3)c1 ; c1cc(-c2ccc3c(c2)C2(c4ccccc4-3)c3ccccc3-c3cc4ccccc4cc32)cc(-c2ccc3c4c(cccc24)-c2ccccc2O3)c1]

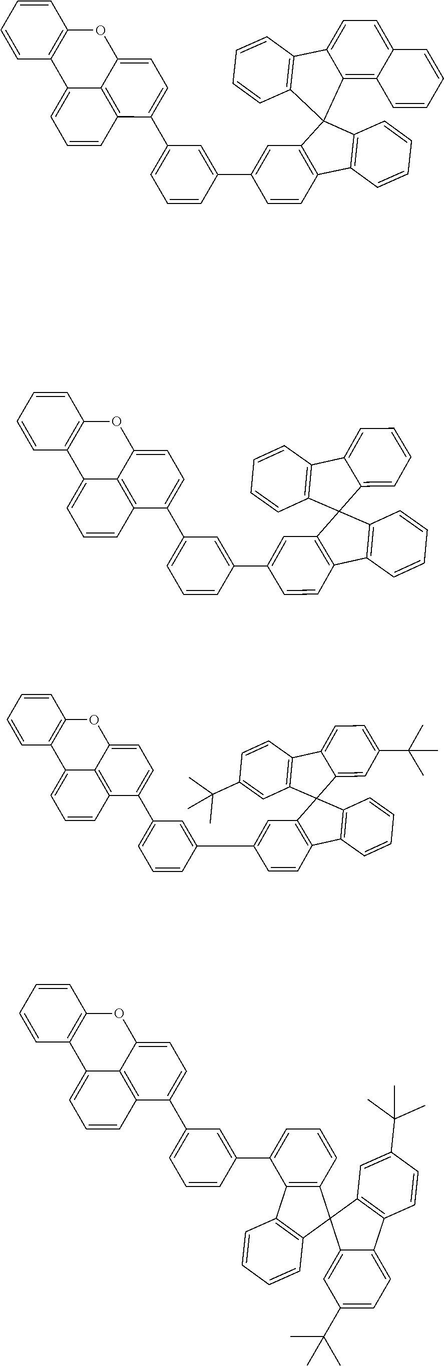 [SMILES: c1cc(-c2ccc3c(c2)C2(c4ccccc4-3)c3ccccc3-c3ccc4ccccc4c32)cc(-c2ccc3c4c(cccc24)-c2ccccc2O3)c1 ; c1cc(-c2ccc3c(c2)C2(c4ccccc4-c4ccccc42)c2ccccc2-3)cc(-c2ccc3c4c(cccc24)-c2ccccc2O3)c1 ; CC(C)(C)c1ccc2c(c1)C1(c3ccccc3-c3ccc(-c4cccc(-c5ccc6c7c(cccc57)-c5ccccc5O6)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4ccc5c6c(cccc46)-c4ccccc4O5)c3)cccc21]

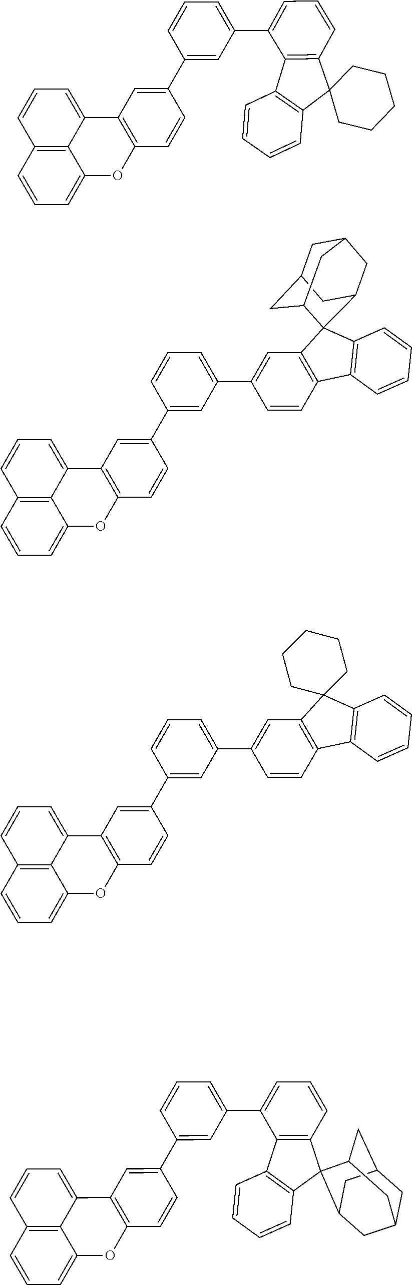 [SMILES: c1cc(-c2ccc3c(c2)-c2cccc4cccc(c24)O3)cc(-c2cccc3c2-c2ccccc2C32CCCCC2)c1 ; c1cc(-c2ccc3c(c2)-c2cccc4cccc(c24)O3)cc(-c2ccc3c(c2)C2(c4ccccc4-3)C3CC4CC(C3)CC2C4)c1 ; c1cc(-c2ccc3c(c2)-c2cccc4cccc(c24)O3)cc(-c2ccc3c(c2)C2(CCCCC2)c2ccccc2-3)c1 ; c1cc(-c2ccc3c(c2)-c2cccc4cccc(c24)O3)cc(-c2cccc3c2-c2ccccc2C32C3CC4CC(C3)CC2C4)c1]

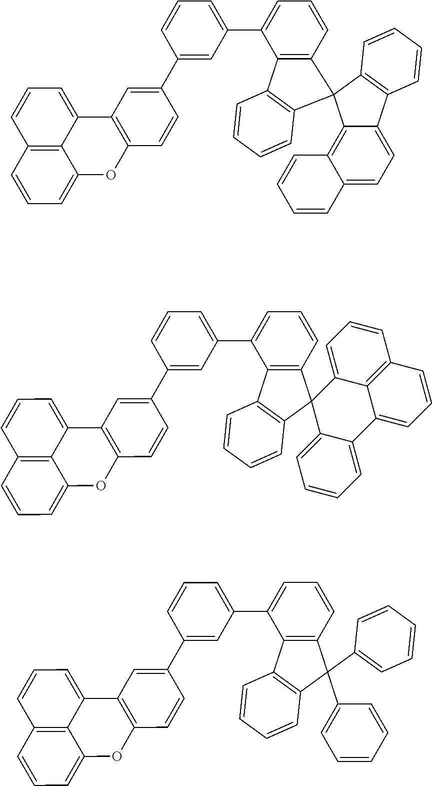 [SMILES: c1cc(-c2ccc3c(c2)-c2cccc4cccc(c24)O3)cc(-c2cccc3c2-c2ccccc2C32c3ccccc3-c3ccc4ccccc4c32)c1 ; c1cc(-c2ccc3c(c2)-c2cccc4cccc(c24)O3)cc(-c2cccc3c2-c2ccccc2C32c3ccccc3-c3cccc4cccc2c34)c1 ; c1ccc(C2(c3ccccc3)c3ccccc3-c3c(-c4cccc(-c5ccc6c(c5)-c5cccc7cccc(c57)O6)c4)cccc32)cc1]

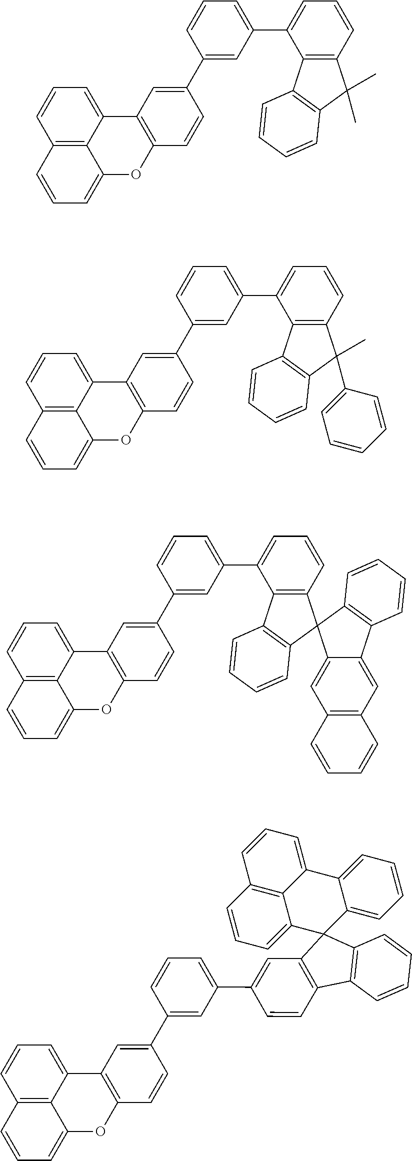 [SMILES: CC1(C)c2ccccc2-c2c(-c3cccc(-c4ccc5c(c4)-c4cccc6cccc(c46)O5)c3)cccc21 ; CC1(c2ccccc2)c2ccccc2-c2c(-c3cccc(-c4ccc5c(c4)-c4cccc6cccc(c46)O5)c3)cccc21 ; c1cc(-c2ccc3c(c2)-c2cccc4cccc(c24)O3)cc(-c2cccc3c2-c2ccccc2C32c3ccccc3-c3cc4ccccc4cc32)c1 ; c1cc(-c2ccc3c(c2)-c2cccc4cccc(c24)O3)cc(-c2ccc3c(c2)C2(c4ccccc4-3)c3ccccc3-c3cccc4cccc2c34)c1]

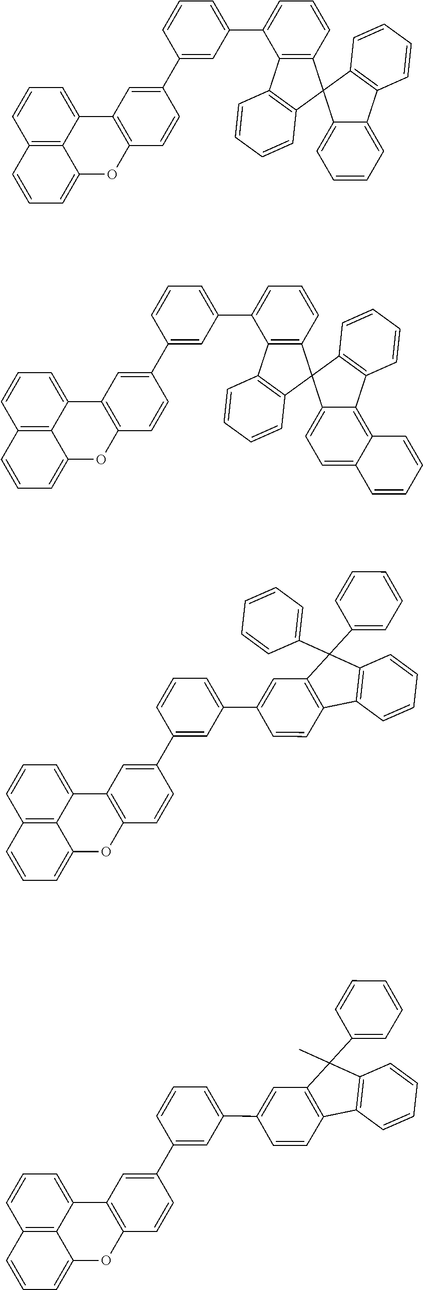 [SMILES: c1cc(-c2ccc3c(c2)-c2cccc4cccc(c24)O3)cc(-c2cccc3c2-c2ccccc2C32c3ccccc3-c3ccccc32)c1 ; c1cc(-c2ccc3c(c2)-c2cccc4cccc(c24)O3)cc(-c2cccc3c2-c2ccccc2C32c3ccccc3-c3c2ccc2ccccc32)c1 ; c1ccc(C2(c3ccccc3)c3ccccc3-c3ccc(-c4cccc(-c5ccc6c(c5)-c5cccc7cccc(c57)O6)c4)cc32)cc1 ; CC1(c2ccccc2)c2ccccc2-c2ccc(-c3cccc(-c4ccc5c(c4)-c4cccc6cccc(c46)O5)c3)cc21]

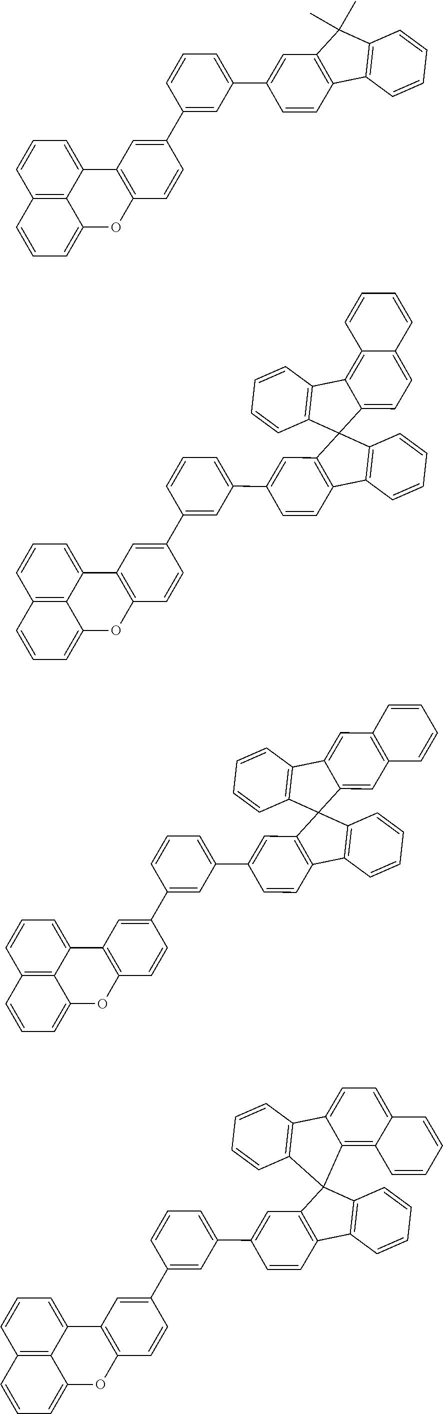 [SMILES: CC1(C)c2ccccc2-c2ccc(-c3cccc(-c4ccc5c(c4)-c4cccc6cccc(c46)O5)c3)cc21 ; c1cc(-c2ccc3c(c2)-c2cccc4cccc(c24)O3)cc(-c2ccc3c(c2)C2(c4ccccc4-3)c3ccccc3-c3c2ccc2ccccc32)c1 ; c1cc(-c2ccc3c(c2)-c2cccc4cccc(c24)O3)cc(-c2ccc3c(c2)C2(c4ccccc4-3)c3ccccc3-c3cc4ccccc4cc32)c1 ; c1cc(-c2ccc3c(c2)-c2cccc4cccc(c24)O3)cc(-c2ccc3c(c2)C2(c4ccccc4-3)c3ccccc3-c3ccc4ccccc4c32)c1]

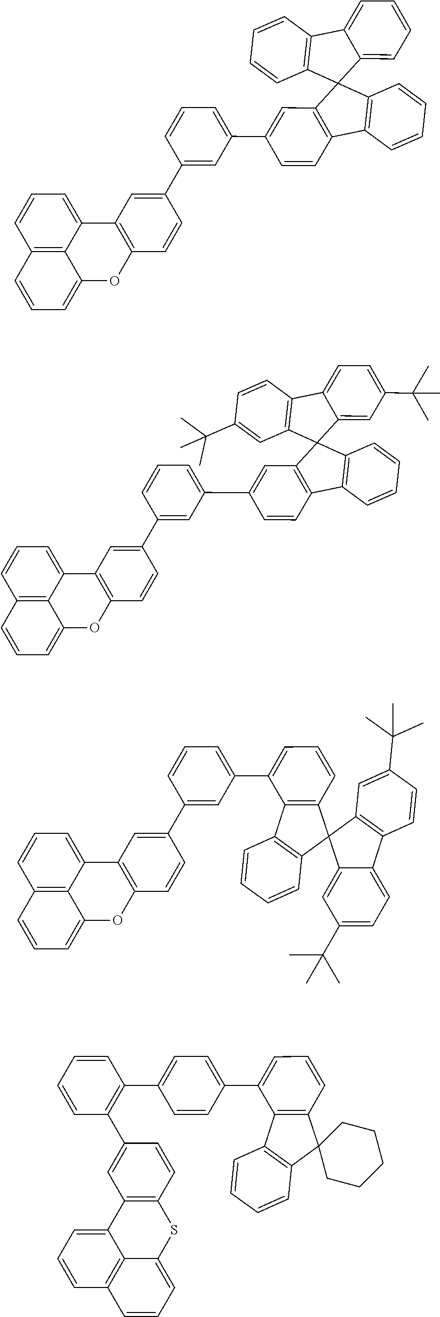 [SMILES: c1cc(-c2ccc3c(c2)-c2cccc4cccc(c24)O3)cc(-c2ccc3c(c2)C2(c4ccccc4-c4ccccc42)c2ccccc2-3)c1 ; CC(C)(C)c1ccc2c(c1)C1(c3ccccc3-c3ccc(-c4cccc(-c5ccc6c(c5)-c5cccc7cccc(c57)O6)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4ccc5c(c4)-c4cccc6cccc(c46)O5)c3)cccc21 ; c1ccc(-c2ccc3c(c2)-c2cccc4cccc(c24)S3)c(-c2ccc(-c3cccc4c3-c3ccccc3C43CCCCC3)cc2)c1]

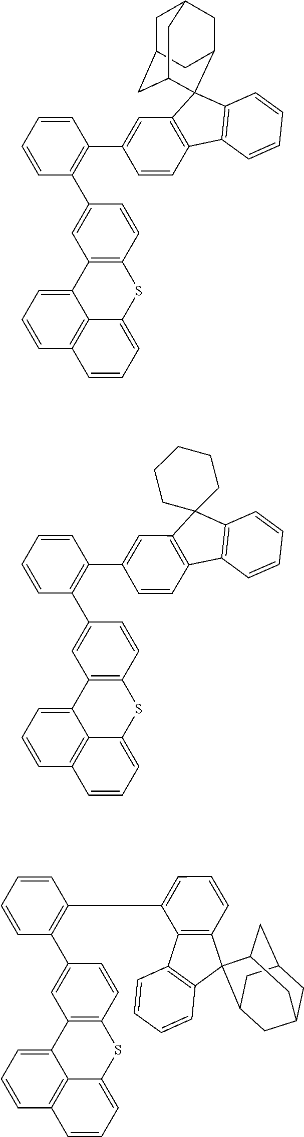 [SMILES: c1ccc(-c2ccc3c(c2)C2(c4ccccc4-3)C3CC4CC(C3)CC2C4)c(-c2ccc3c(c2)-c2cccc4cccc(c24)S3)c1 ; c1ccc(-c2ccc3c(c2)C2(CCCCC2)c2ccccc2-3)c(-c2ccc3c(c2)-c2cccc4cccc(c24)S3)c1 ; c1ccc(-c2cccc3c2-c2ccccc2C32C3CC4CC(C3)CC2C4)c(-c2ccc3c(c2)-c2cccc4cccc(c24)S3)c1]

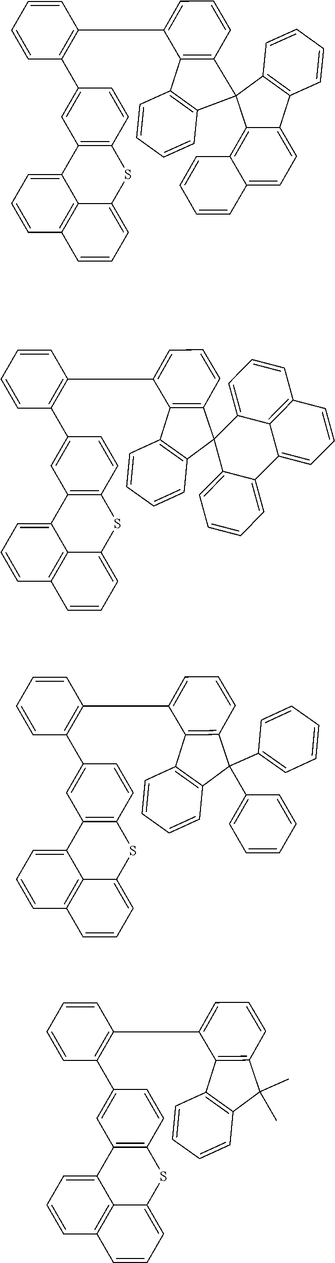 [SMILES: c1ccc(-c2cccc3c2-c2ccccc2C32c3ccccc3-c3ccc4ccccc4c32)c(-c2ccc3c(c2)-c2cccc4cccc(c24)S3)c1 ; c1ccc(-c2cccc3c2-c2ccccc2C32c3ccccc3-c3cccc4cccc2c34)c(-c2ccc3c(c2)-c2cccc4cccc(c24)S3)c1 ; c1ccc(C2(c3ccccc3)c3ccccc3-c3c(-c4ccccc4-c4ccc5c(c4)-c4cccc6cccc(c46)S5)cccc32)cc1 ; CC1(C)c2ccccc2-c2c(-c3ccccc3-c3ccc4c(c3)-c3cccc5cccc(c35)S4)cccc21]

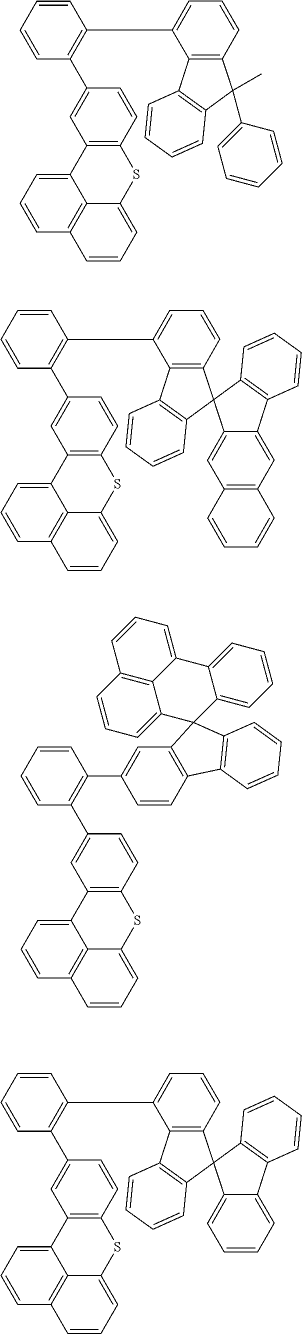 [SMILES: CC1(c2ccccc2)c2ccccc2-c2c(-c3ccccc3-c3ccc4c(c3)-c3cccc5cccc(c35)S4)cccc21 ; c1ccc(-c2cccc3c2-c2ccccc2C32c3ccccc3-c3cc4ccccc4cc32)c(-c2ccc3c(c2)-c2cccc4cccc(c24)S3)c1 ; c1ccc(-c2ccc3c(c2)C2(c4ccccc4-3)c3ccccc3-c3cccc4cccc2c34)c(-c2ccc3c(c2)-c2cccc4cccc(c24)S3)c1 ; c1ccc(-c2cccc3c2-c2ccccc2C32c3ccccc3-c3ccccc32)c(-c2ccc3c(c2)-c2cccc4cccc(c24)S3)c1]

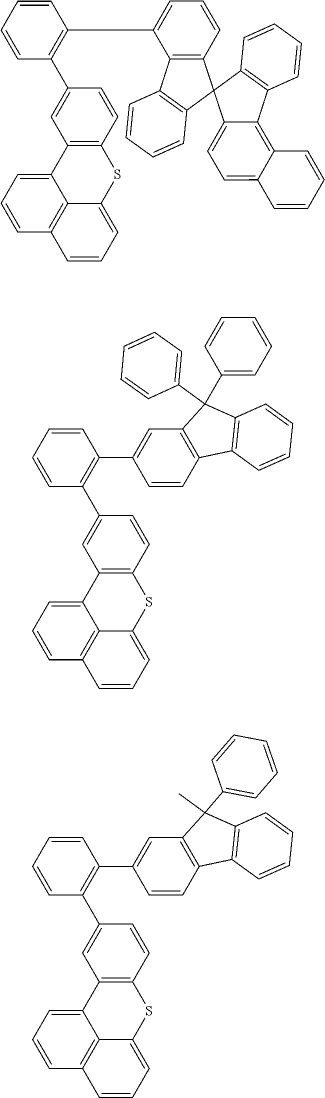 [SMILES: c1ccc(-c2cccc3c2-c2ccccc2C32c3ccccc3-c3c2ccc2ccccc32)c(-c2ccc3c(c2)-c2cccc4cccc(c24)S3)c1 ; c1ccc(C2(c3ccccc3)c3ccccc3-c3ccc(-c4ccccc4-c4ccc5c(c4)-c4cccc6cccc(c46)S5)cc32)cc1 ; CC1(c2ccccc2)c2ccccc2-c2ccc(-c3ccccc3-c3ccc4c(c3)-c3cccc5cccc(c35)S4)cc21]

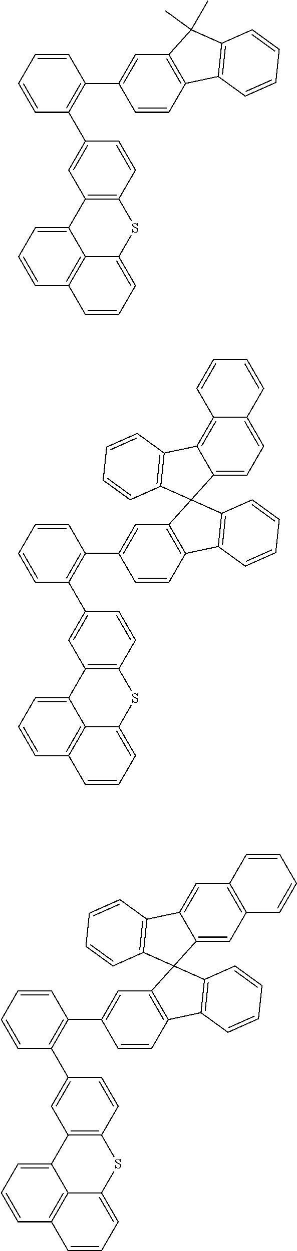 [SMILES: CC1(C)c2ccccc2-c2ccc(-c3ccccc3-c3ccc4c(c3)-c3cccc5cccc(c35)S4)cc21 ; c1ccc(-c2ccc3c(c2)C2(c4ccccc4-3)c3ccccc3-c3c2ccc2ccccc32)c(-c2ccc3c(c2)-c2cccc4cccc(c24)S3)c1 ; c1ccc(-c2ccc3c(c2)C2(c4ccccc4-3)c3ccccc3-c3cc4ccccc4cc32)c(-c2ccc3c(c2)-c2cccc4cccc(c24)S3)c1]

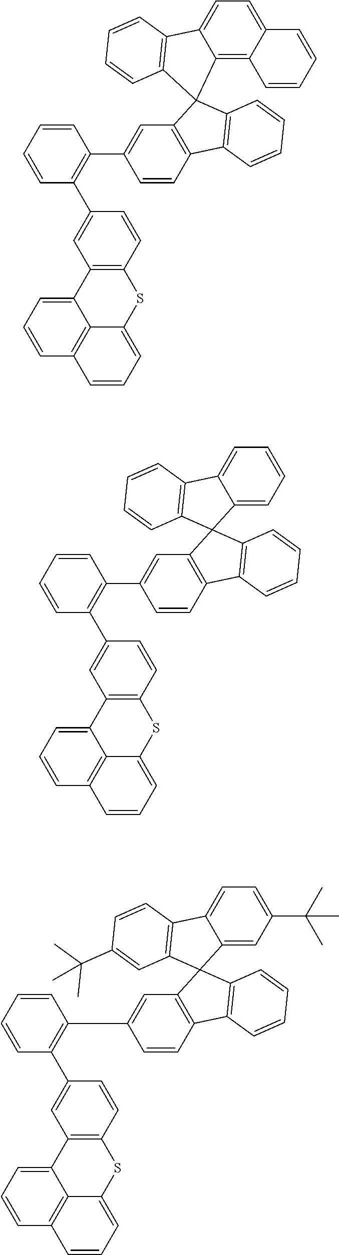 [SMILES: c1ccc(-c2ccc3c(c2)C2(c4ccccc4-3)c3ccccc3-c3ccc4ccccc4c32)c(-c2ccc3c(c2)-c2cccc4cccc(c24)S3)c1 ; c1ccc(-c2ccc3c(c2)C2(c4ccccc4-c4ccccc42)c2ccccc2-3)c(-c2ccc3c(c2)-c2cccc4cccc(c24)S3)c1 ; CC(C)(C)c1ccc2c(c1)C1(c3ccccc3-c3ccc(-c4ccccc4-c4ccc5c(c4)-c4cccc6cccc(c46)S5)cc31)c1cc(C(C)(C)C)ccc1-2]

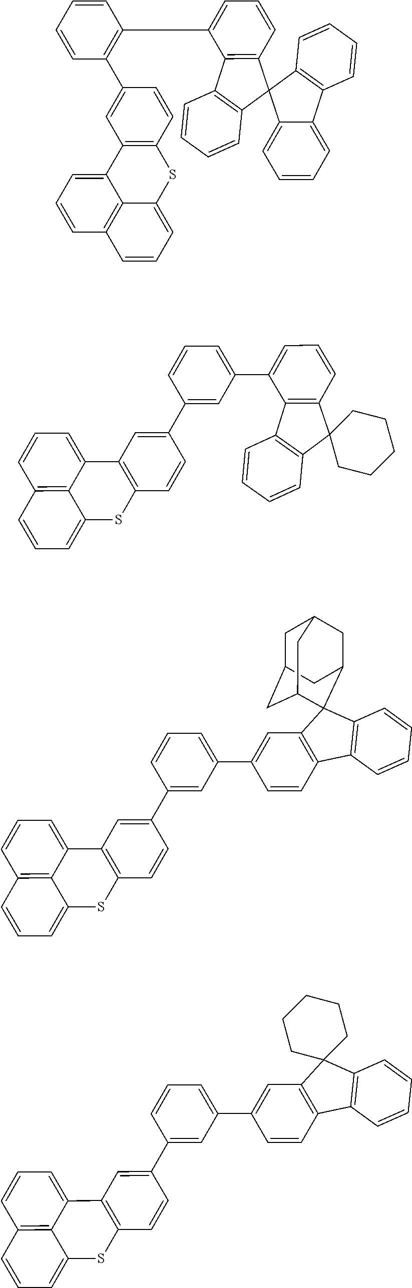 [SMILES: c1ccc(-c2cccc3c2-c2ccccc2C32c3ccccc3-c3ccccc32)c(-c2ccc3c(c2)-c2cccc4cccc(c24)S3)c1 ; c1cc(-c2ccc3c(c2)-c2cccc4cccc(c24)S3)cc(-c2cccc3c2-c2ccccc2C32CCCCC2)c1 ; c1cc(-c2ccc3c(c2)-c2cccc4cccc(c24)S3)cc(-c2ccc3c(c2)C2(c4ccccc4-3)C3CC4CC(C3)CC2C4)c1 ; c1cc(-c2ccc3c(c2)-c2cccc4cccc(c24)S3)cc(-c2ccc3c(c2)C2(CCCCC2)c2ccccc2-3)c1]

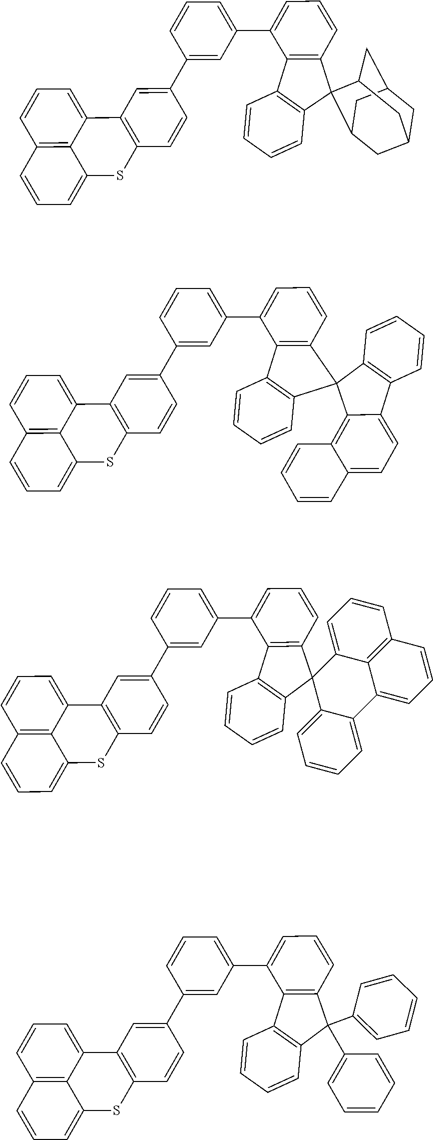 [SMILES: c1cc(-c2ccc3c(c2)-c2cccc4cccc(c24)S3)cc(-c2cccc3c2-c2ccccc2C32C3CC4CC(C3)CC2C4)c1 ; c1cc(-c2ccc3c(c2)-c2cccc4cccc(c24)S3)cc(-c2cccc3c2-c2ccccc2C32c3ccccc3-c3ccc4ccccc4c32)c1 ; c1cc(-c2ccc3c(c2)-c2cccc4cccc(c24)S3)cc(-c2cccc3c2-c2ccccc2C32c3ccccc3-c3cccc4cccc2c34)c1 ; c1ccc(C2(c3ccccc3)c3ccccc3-c3c(-c4cccc(-c5ccc6c(c5)-c5cccc7cccc(c57)S6)c4)cccc32)cc1]

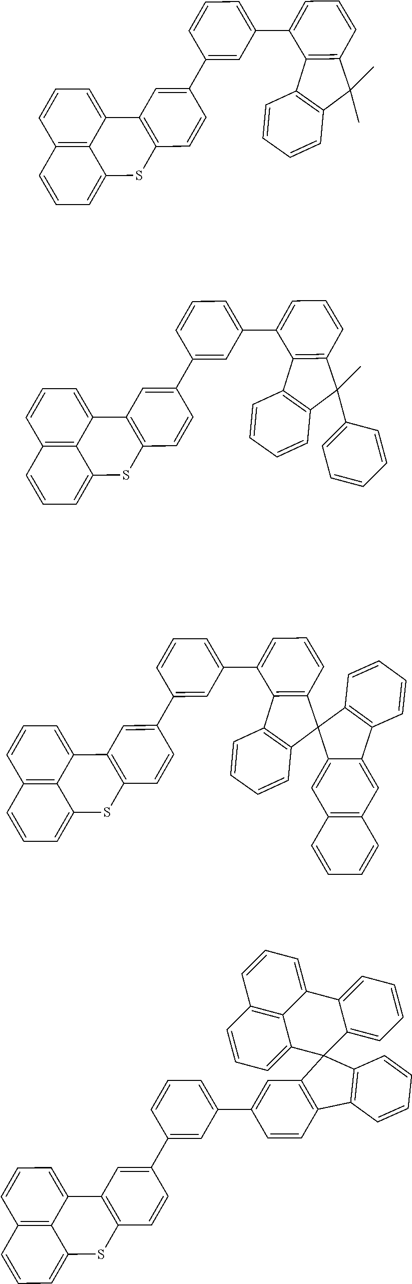 [SMILES: CC1(C)c2ccccc2-c2c(-c3cccc(-c4ccc5c(c4)-c4cccc6cccc(c46)S5)c3)cccc21 ; CC1(c2ccccc2)c2ccccc2-c2c(-c3cccc(-c4ccc5c(c4)-c4cccc6cccc(c46)S5)c3)cccc21 ; c1cc(-c2ccc3c(c2)-c2cccc4cccc(c24)S3)cc(-c2cccc3c2-c2ccccc2C32c3ccccc3-c3cc4ccccc4cc32)c1 ; c1cc(-c2ccc3c(c2)-c2cccc4cccc(c24)S3)cc(-c2ccc3c(c2)C2(c4ccccc4-3)c3ccccc3-c3cccc4cccc2c34)c1]

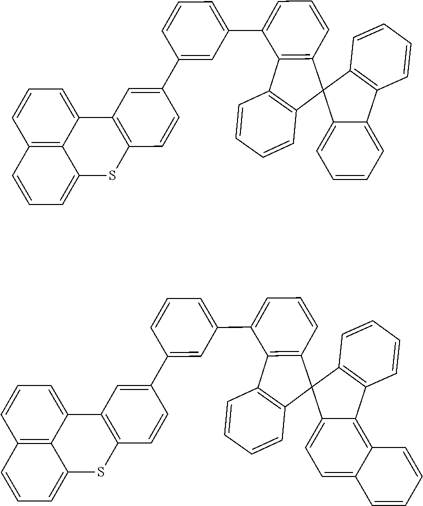 [SMILES: c1cc(-c2ccc3c(c2)-c2cccc4cccc(c24)S3)cc(-c2cccc3c2-c2ccccc2C32c3ccccc3-c3ccccc32)c1 ; c1cc(-c2ccc3c(c2)-c2cccc4cccc(c24)S3)cc(-c2cccc3c2-c2ccccc2C32c3ccccc3-c3c2ccc2ccccc32)c1]

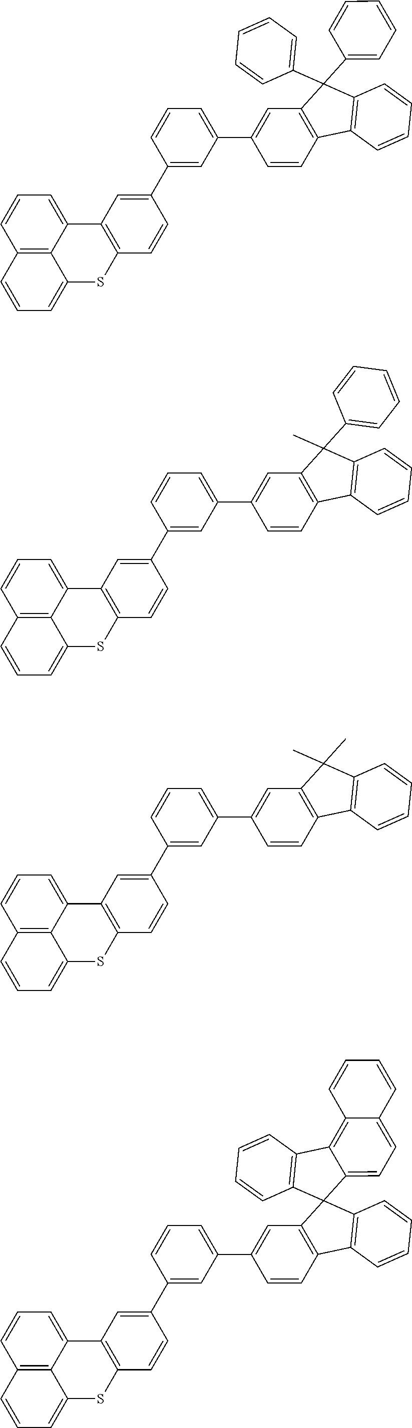 [SMILES: c1ccc(C2(c3ccccc3)c3ccccc3-c3ccc(-c4cccc(-c5ccc6c(c5)-c5cccc7cccc(c57)S6)c4)cc32)cc1 ; CC1(c2ccccc2)c2ccccc2-c2ccc(-c3cccc(-c4ccc5c(c4)-c4cccc6cccc(c46)S5)c3)cc21 ; CC1(C)c2ccccc2-c2ccc(-c3cccc(-c4ccc5c(c4)-c4cccc6cccc(c46)S5)c3)cc21 ; c1cc(-c2ccc3c(c2)-c2cccc4cccc(c24)S3)cc(-c2ccc3c(c2)C2(c4ccccc4-3)c3ccccc3-c3c2ccc2ccccc32)c1]

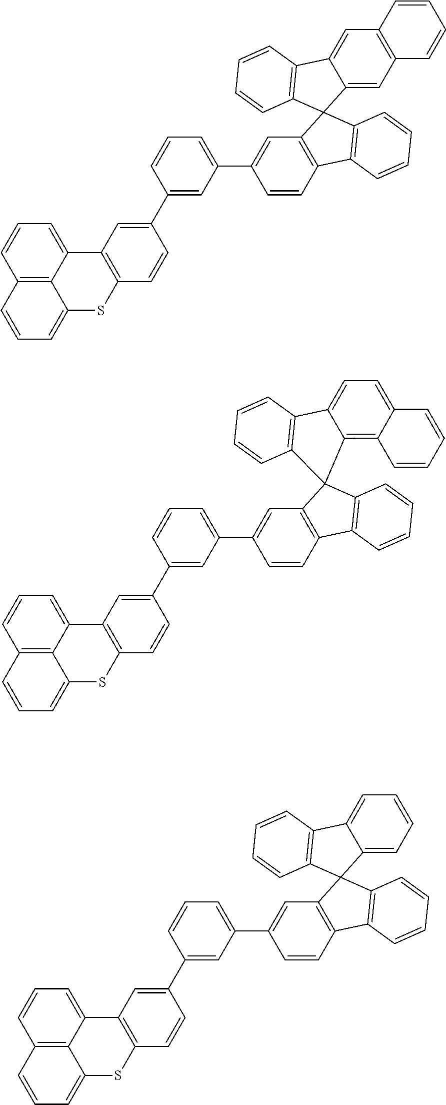 [SMILES: c1cc(-c2ccc3c(c2)-c2cccc4cccc(c24)S3)cc(-c2ccc3c(c2)C2(c4ccccc4-3)c3ccccc3-c3cc4ccccc4cc32)c1 ; c1cc(-c2ccc3c(c2)-c2cccc4cccc(c24)S3)cc(-c2ccc3c(c2)C2(c4ccccc4-3)c3ccccc3-c3ccc4ccccc4c32)c1 ; c1cc(-c2ccc3c(c2)-c2cccc4cccc(c24)S3)cc(-c2ccc3c(c2)C2(c4ccccc4-c4ccccc42)c2ccccc2-3)c1]

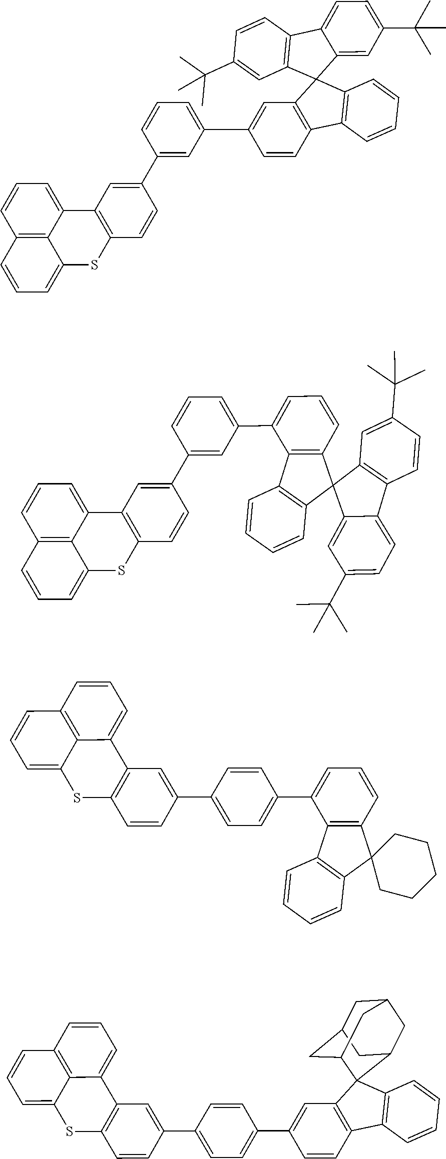 [SMILES: CC(C)(C)c1ccc2c(c1)C1(c3ccccc3-c3ccc(-c4cccc(-c5ccc6c(c5)-c5cccc7cccc(c57)S6)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4ccc5c(c4)-c4cccc6cccc(c46)S5)c3)cccc21 ; c1ccc2c(c1)-c1c(-c3ccc(-c4ccc5c(c4)-c4cccc6cccc(c46)S5)cc3)cccc1C21CCCCC1 ; c1ccc2c(c1)-c1ccc(-c3ccc(-c4ccc5c(c4)-c4cccc6cccc(c46)S5)cc3)cc1C21C2CC3CC(C2)CC1C3]

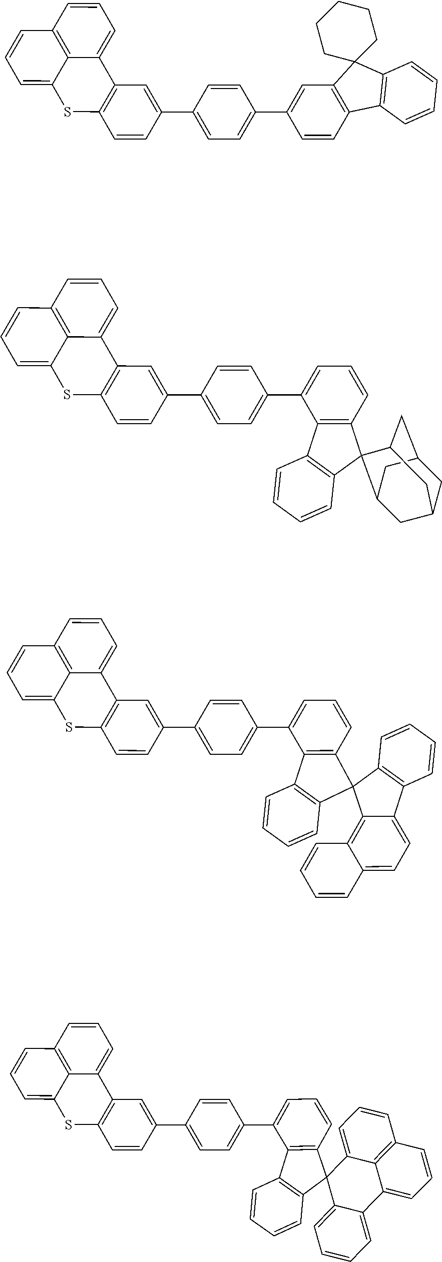 [SMILES: c1ccc2c(c1)-c1ccc(-c3ccc(-c4ccc5c(c4)-c4cccc6cccc(c46)S5)cc3)cc1C21CCCCC1 ; c1ccc2c(c1)-c1c(-c3ccc(-c4ccc5c(c4)-c4cccc6cccc(c46)S5)cc3)cccc1C21C2CC3CC(C2)CC1C3 ; c1ccc2c(c1)-c1ccc3ccccc3c1C21c2ccccc2-c2c(-c3ccc(-c4ccc5c(c4)-c4cccc6cccc(c46)S5)cc3)cccc21 ; c1ccc2c(c1)-c1c(-c3ccc(-c4ccc5c(c4)-c4cccc6cccc(c46)S5)cc3)cccc1C21c2ccccc2-c2cccc3cccc1c23]

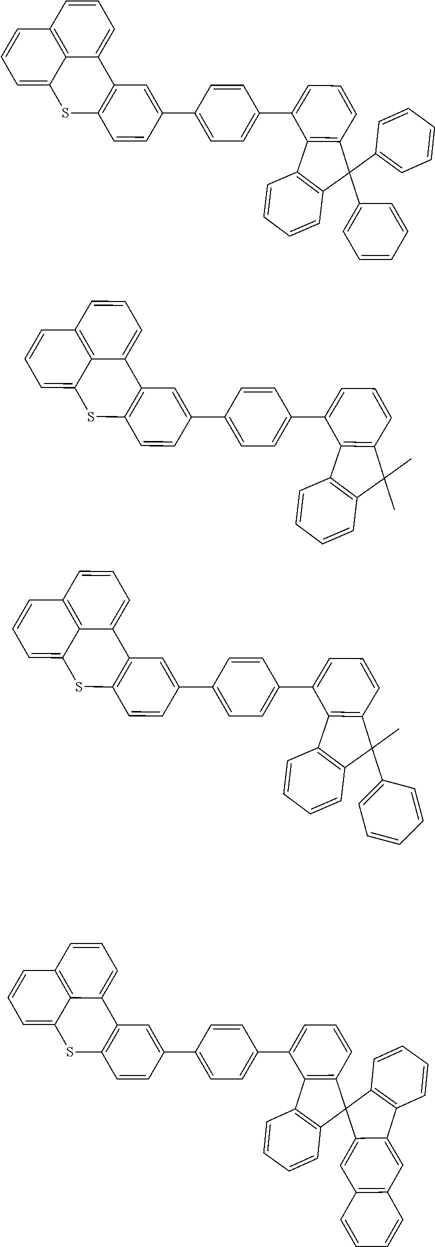 [SMILES: c1ccc(C2(c3ccccc3)c3ccccc3-c3c(-c4ccc(-c5ccc6c(c5)-c5cccc7cccc(c57)S6)cc4)cccc32)cc1 ; CC1(C)c2ccccc2-c2c(-c3ccc(-c4ccc5c(c4)-c4cccc6cccc(c46)S5)cc3)cccc21 ; CC1(c2ccccc2)c2ccccc2-c2c(-c3ccc(-c4ccc5c(c4)-c4cccc6cccc(c46)S5)cc3)cccc21 ; c1ccc2c(c1)-c1cc3ccccc3cc1C21c2ccccc2-c2c(-c3ccc(-c4ccc5c(c4)-c4cccc6cccc(c46)S5)cc3)cccc21]

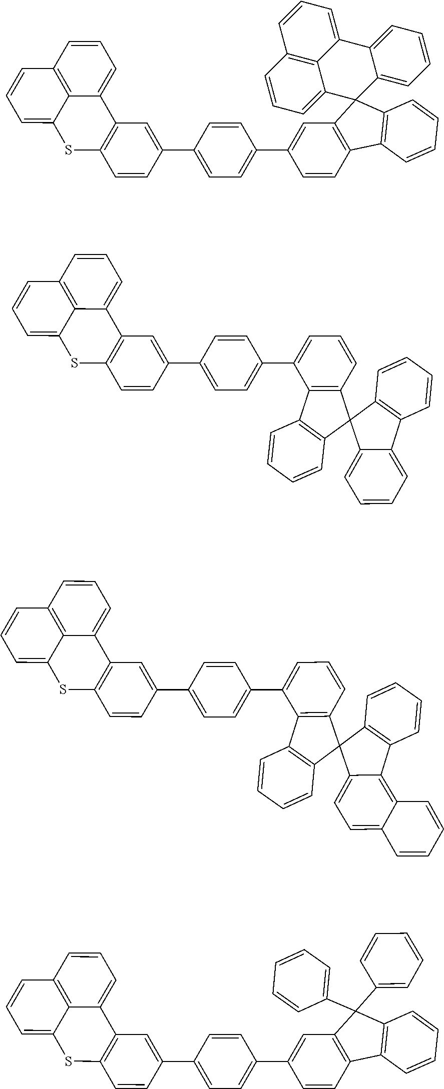 [SMILES: c1ccc2c(c1)-c1ccc(-c3ccc(-c4ccc5c(c4)-c4cccc6cccc(c46)S5)cc3)cc1C21c2ccccc2-c2cccc3cccc1c23 ; c1ccc2c(c1)-c1ccccc1C21c2ccccc2-c2c(-c3ccc(-c4ccc5c(c4)-c4cccc6cccc(c46)S5)cc3)cccc21 ; c1ccc2c(c1)-c1c(-c3ccc(-c4ccc5c(c4)-c4cccc6cccc(c46)S5)cc3)cccc1C21c2ccccc2-c2c1ccc1ccccc21 ; c1ccc(C2(c3ccccc3)c3ccccc3-c3ccc(-c4ccc(-c5ccc6c(c5)-c5cccc7cccc(c57)S6)cc4)cc32)cc1]

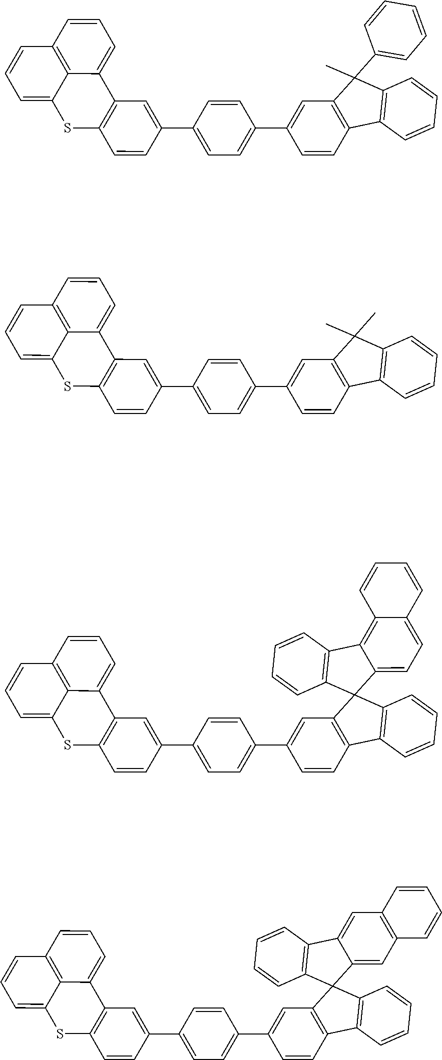 [SMILES: CC1(c2ccccc2)c2ccccc2-c2ccc(-c3ccc(-c4ccc5c(c4)-c4cccc6cccc(c46)S5)cc3)cc21 ; CC1(C)c2ccccc2-c2ccc(-c3ccc(-c4ccc5c(c4)-c4cccc6cccc(c46)S5)cc3)cc21 ; c1ccc2c(c1)-c1ccc(-c3ccc(-c4ccc5c(c4)-c4cccc6cccc(c46)S5)cc3)cc1C21c2ccccc2-c2c1ccc1ccccc21 ; c1ccc2c(c1)-c1ccc(-c3ccc(-c4ccc5c(c4)-c4cccc6cccc(c46)S5)cc3)cc1C21c2ccccc2-c2cc3ccccc3cc21]

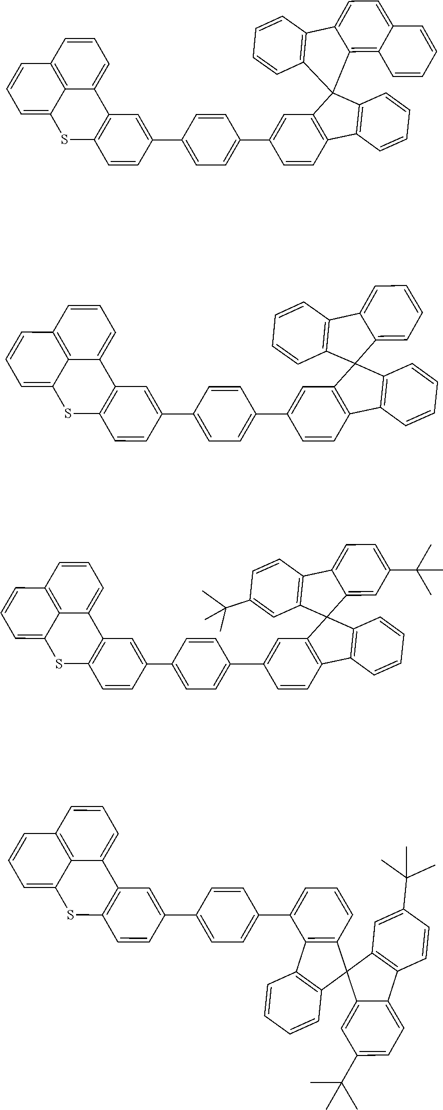 [SMILES: c1ccc2c(c1)-c1ccc(-c3ccc(-c4ccc5c(c4)-c4cccc6cccc(c46)S5)cc3)cc1C21c2ccccc2-c2ccc3ccccc3c21 ; c1ccc2c(c1)-c1ccccc1C21c2ccccc2-c2ccc(-c3ccc(-c4ccc5c(c4)-c4cccc6cccc(c46)S5)cc3)cc21 ; CC(C)(C)c1ccc2c(c1)C1(c3ccccc3-c3ccc(-c4ccc(-c5ccc6c(c5)-c5cccc7cccc(c57)S6)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4ccc5c(c4)-c4cccc6cccc(c46)S5)cc3)cccc21]

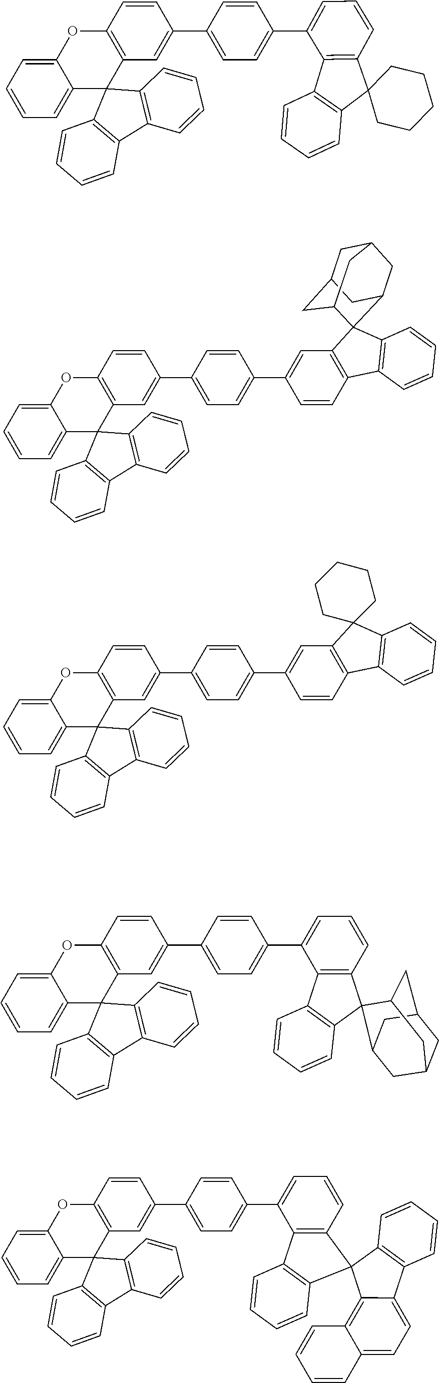 [SMILES: c1ccc2c(c1)Oc1ccc(-c3ccc(-c4cccc5c4-c4ccccc4C54CCCCC4)cc3)cc1C21c2ccccc2-c2ccccc21 ; c1ccc2c(c1)Oc1ccc(-c3ccc(-c4ccc5c(c4)C4(c6ccccc6-5)C5CC6CC(C5)CC4C6)cc3)cc1C21c2ccccc2-c2ccccc21 ; c1ccc2c(c1)Oc1ccc(-c3ccc(-c4ccc5c(c4)C4(CCCCC4)c4ccccc4-5)cc3)cc1C21c2ccccc2-c2ccccc21 ; c1ccc2c(c1)Oc1ccc(-c3ccc(-c4cccc5c4-c4ccccc4C54C5CC6CC(C5)CC4C6)cc3)cc1C21c2ccccc2-c2ccccc21 ; c1ccc2c(c1)Oc1ccc(-c3ccc(-c4cccc5c4-c4ccccc4C54c5ccccc5-c5ccc6ccccc6c54)cc3)cc1C21c2ccccc2-c2ccccc21]

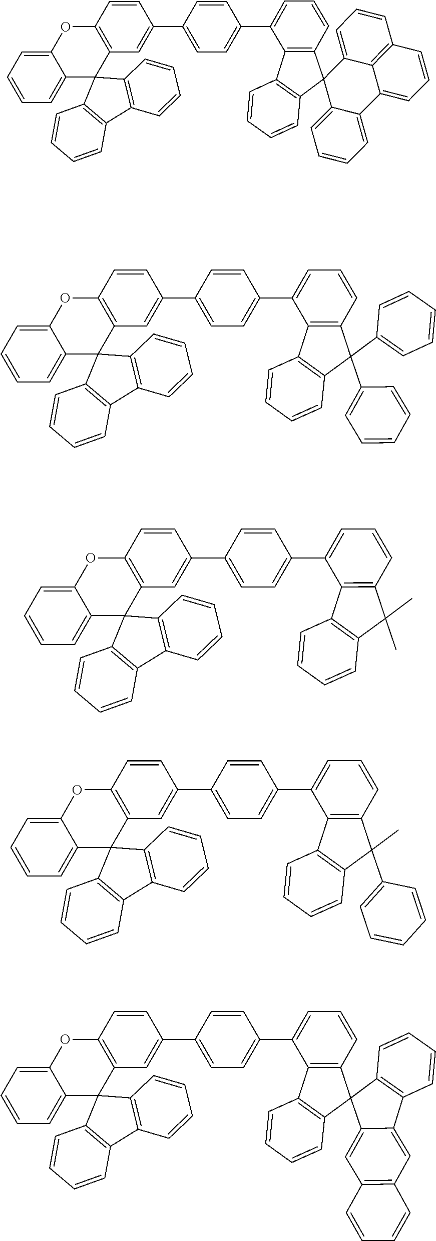 [SMILES: c1ccc2c(c1)Oc1ccc(-c3ccc(-c4cccc5c4-c4ccccc4C54c5ccccc5-c5cccc6cccc4c56)cc3)cc1C21c2ccccc2-c2ccccc21 ; c1ccc(C2(c3ccccc3)c3ccccc3-c3c(-c4ccc(-c5ccc6c(c5)C5(c7ccccc7O6)c6ccccc6-c6ccccc65)cc4)cccc32)cc1 ; CC1(C)c2ccccc2-c2c(-c3ccc(-c4ccc5c(c4)C4(c6ccccc6O5)c5ccccc5-c5ccccc54)cc3)cccc21 ; CC1(c2ccccc2)c2ccccc2-c2c(-c3ccc(-c4ccc5c(c4)C4(c6ccccc6O5)c5ccccc5-c5ccccc54)cc3)cccc21 ; c1ccc2c(c1)Oc1ccc(-c3ccc(-c4cccc5c4-c4ccccc4C54c5ccccc5-c5cc6ccccc6cc54)cc3)cc1C21c2ccccc2-c2ccccc21]

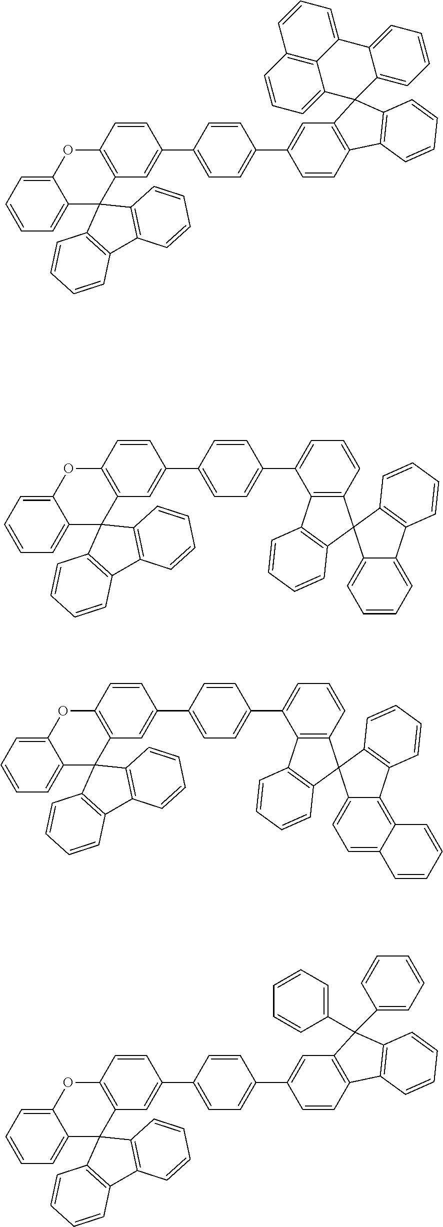 [SMILES: c1ccc2c(c1)Oc1ccc(-c3ccc(-c4ccc5c(c4)C4(c6ccccc6-5)c5ccccc5-c5cccc6cccc4c56)cc3)cc1C21c2ccccc2-c2ccccc21 ; c1ccc2c(c1)Oc1ccc(-c3ccc(-c4cccc5c4-c4ccccc4C54c5ccccc5-c5ccccc54)cc3)cc1C21c2ccccc2-c2ccccc21 ; c1ccc2c(c1)Oc1ccc(-c3ccc(-c4cccc5c4-c4ccccc4C54c5ccccc5-c5c4ccc4ccccc54)cc3)cc1C21c2ccccc2-c2ccccc21 ; c1ccc(C2(c3ccccc3)c3ccccc3-c3ccc(-c4ccc(-c5ccc6c(c5)C5(c7ccccc7O6)c6ccccc6-c6ccccc65)cc4)cc32)cc1]

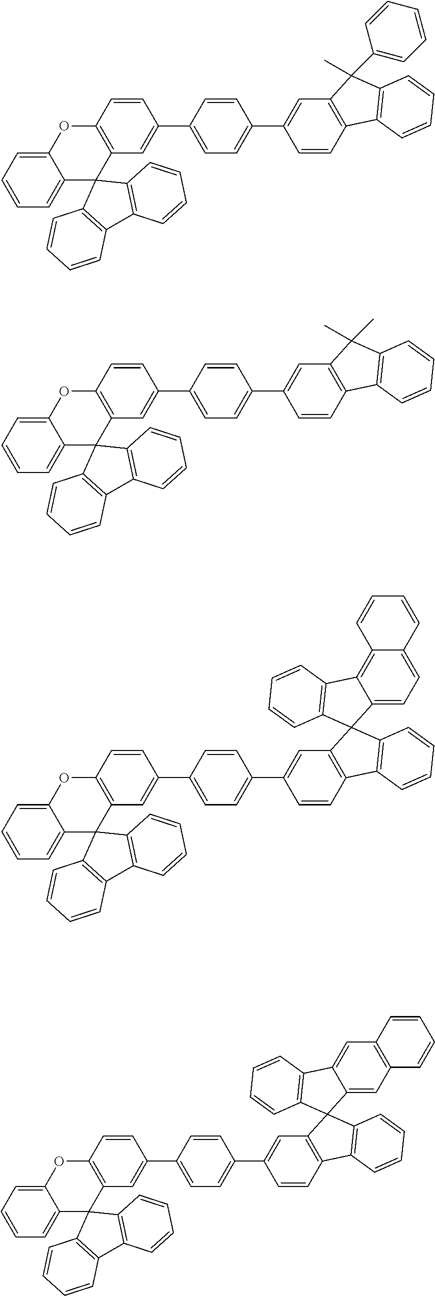 [SMILES: CC1(c2ccccc2)c2ccccc2-c2ccc(-c3ccc(-c4ccc5c(c4)C4(c6ccccc6O5)c5ccccc5-c5ccccc54)cc3)cc21 ; CC1(C)c2ccccc2-c2ccc(-c3ccc(-c4ccc5c(c4)C4(c6ccccc6O5)c5ccccc5-c5ccccc54)cc3)cc21 ; c1ccc2c(c1)Oc1ccc(-c3ccc(-c4ccc5c(c4)C4(c6ccccc6-5)c5ccccc5-c5c4ccc4ccccc54)cc3)cc1C21c2ccccc2-c2ccccc21 ; c1ccc2c(c1)Oc1ccc(-c3ccc(-c4ccc5c(c4)C4(c6ccccc6-5)c5ccccc5-c5cc6ccccc6cc54)cc3)cc1C21c2ccccc2-c2ccccc21]

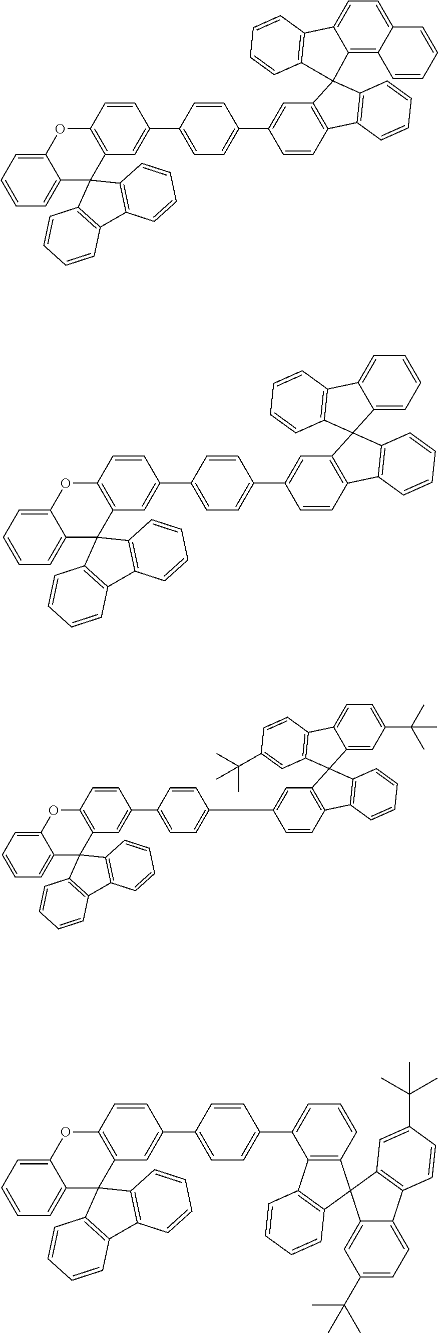 [SMILES: c1ccc2c(c1)Oc1ccc(-c3ccc(-c4ccc5c(c4)C4(c6ccccc6-5)c5ccccc5-c5ccc6ccccc6c54)cc3)cc1C21c2ccccc2-c2ccccc21 ; c1ccc2c(c1)Oc1ccc(-c3ccc(-c4ccc5c(c4)C4(c6ccccc6-c6ccccc64)c4ccccc4-5)cc3)cc1C21c2ccccc2-c2ccccc21 ; CC(C)(C)c1ccc2c(c1)C1(c3ccccc3-c3ccc(-c4ccc(-c5ccc6c(c5)C5(c7ccccc7O6)c6ccccc6-c6ccccc65)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4ccc5c(c4)C4(c6ccccc6O5)c5ccccc5-c5ccccc54)cc3)cccc21]

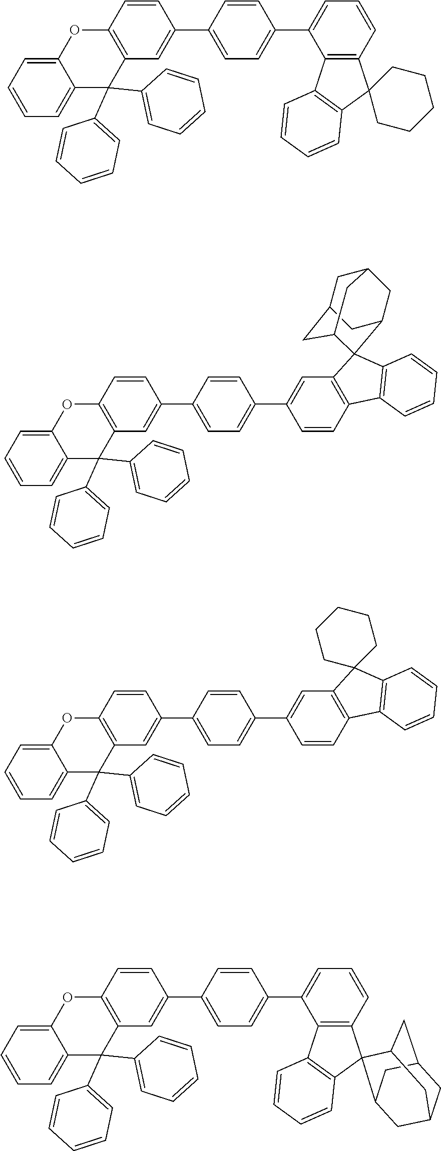 [SMILES: c1ccc(C2(c3ccccc3)c3ccccc3Oc3ccc(-c4ccc(-c5cccc6c5-c5ccccc5C65CCCCC5)cc4)cc32)cc1 ; c1ccc(C2(c3ccccc3)c3ccccc3Oc3ccc(-c4ccc(-c5ccc6c(c5)C5(c7ccccc7-6)C6CC7CC(C6)CC5C7)cc4)cc32)cc1 ; c1ccc(C2(c3ccccc3)c3ccccc3Oc3ccc(-c4ccc(-c5ccc6c(c5)C5(CCCCC5)c5ccccc5-6)cc4)cc32)cc1 ; c1ccc(C2(c3ccccc3)c3ccccc3Oc3ccc(-c4ccc(-c5cccc6c5-c5ccccc5C65C6CC7CC(C6)CC5C7)cc4)cc32)cc1]

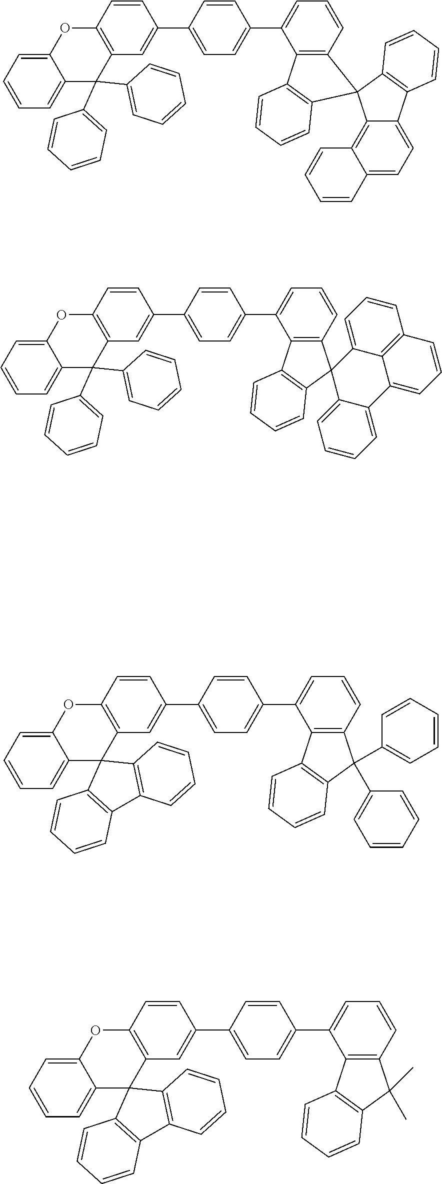 [SMILES: c1ccc(C2(c3ccccc3)c3ccccc3Oc3ccc(-c4ccc(-c5cccc6c5-c5ccccc5C65c6ccccc6-c6ccc7ccccc7c65)cc4)cc32)cc1 ; c1ccc(C2(c3ccccc3)c3ccccc3Oc3ccc(-c4ccc(-c5cccc6c5-c5ccccc5C65c6ccccc6-c6cccc7cccc5c67)cc4)cc32)cc1 ; c1ccc(C2(c3ccccc3)c3ccccc3-c3c(-c4ccc(-c5ccc6c(c5)C5(c7ccccc7O6)c6ccccc6-c6ccccc65)cc4)cccc32)cc1 ; CC1(C)c2ccccc2-c2c(-c3ccc(-c4ccc5c(c4)C4(c6ccccc6O5)c5ccccc5-c5ccccc54)cc3)cccc21]

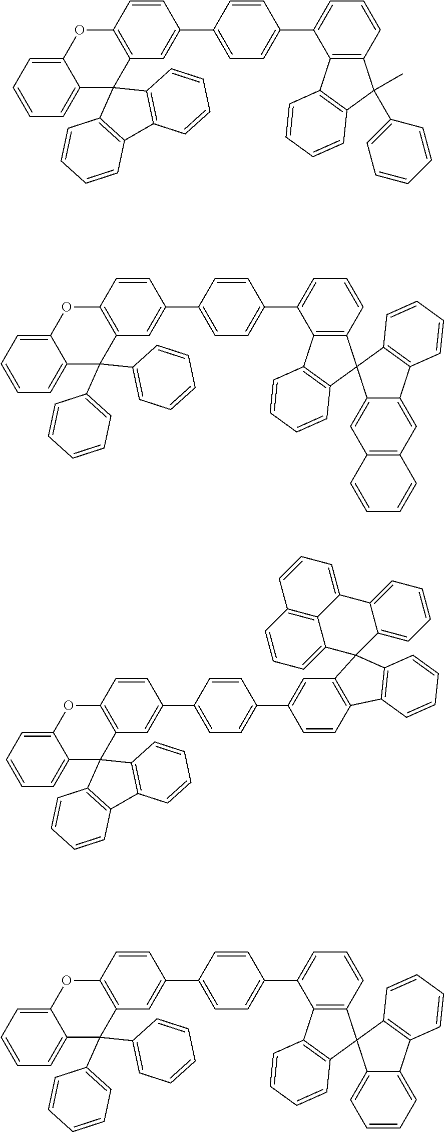 [SMILES: CC1(c2ccccc2)c2ccccc2-c2c(-c3ccc(-c4ccc5c(c4)C4(c6ccccc6O5)c5ccccc5-c5ccccc54)cc3)cccc21 ; c1ccc(C2(c3ccccc3)c3ccccc3Oc3ccc(-c4ccc(-c5cccc6c5-c5ccccc5C65c6ccccc6-c6cc7ccccc7cc65)cc4)cc32)cc1 ; c1ccc2c(c1)Oc1ccc(-c3ccc(-c4ccc5c(c4)C4(c6ccccc6-5)c5ccccc5-c5cccc6cccc4c56)cc3)cc1C21c2ccccc2-c2ccccc21 ; c1ccc(C2(c3ccccc3)c3ccccc3Oc3ccc(-c4ccc(-c5cccc6c5-c5ccccc5C65c6ccccc6-c6ccccc65)cc4)cc32)cc1]

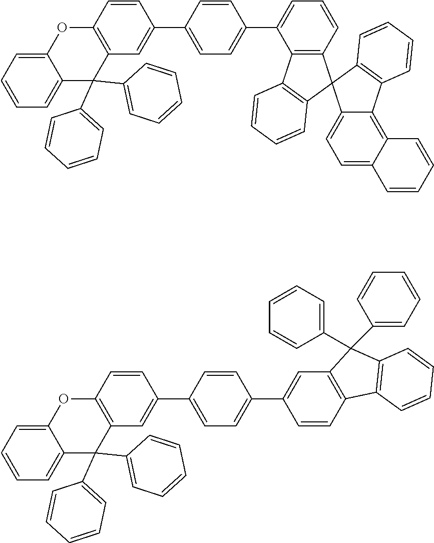 [SMILES: c1ccc(C2(c3ccccc3)c3ccccc3Oc3ccc(-c4ccc(-c5cccc6c5-c5ccccc5C65c6ccccc6-c6c5ccc5ccccc65)cc4)cc32)cc1 ; c1ccc(C2(c3ccccc3)c3ccccc3Oc3ccc(-c4ccc(-c5ccc6c(c5)C(c5ccccc5)(c5ccccc5)c5ccccc5-6)cc4)cc32)cc1]

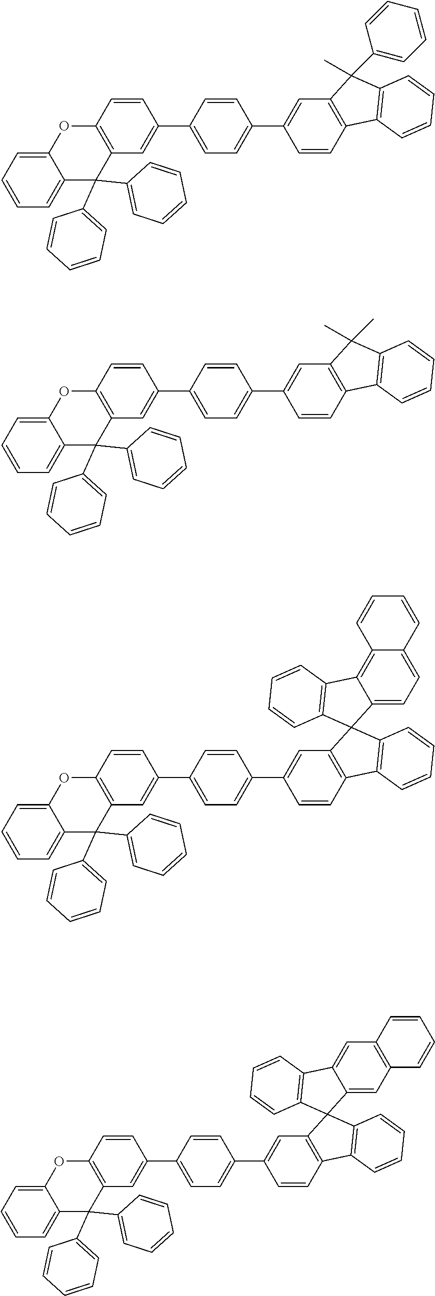 [SMILES: CC1(c2ccccc2)c2ccccc2-c2ccc(-c3ccc(-c4ccc5c(c4)C(c4ccccc4)(c4ccccc4)c4ccccc4O5)cc3)cc21 ; CC1(C)c2ccccc2-c2ccc(-c3ccc(-c4ccc5c(c4)C(c4ccccc4)(c4ccccc4)c4ccccc4O5)cc3)cc21 ; c1ccc(C2(c3ccccc3)c3ccccc3Oc3ccc(-c4ccc(-c5ccc6c(c5)C5(c7ccccc7-6)c6ccccc6-c6c5ccc5ccccc65)cc4)cc32)cc1 ; c1ccc(C2(c3ccccc3)c3ccccc3Oc3ccc(-c4ccc(-c5ccc6c(c5)C5(c7ccccc7-6)c6ccccc6-c6cc7ccccc7cc65)cc4)cc32)cc1]

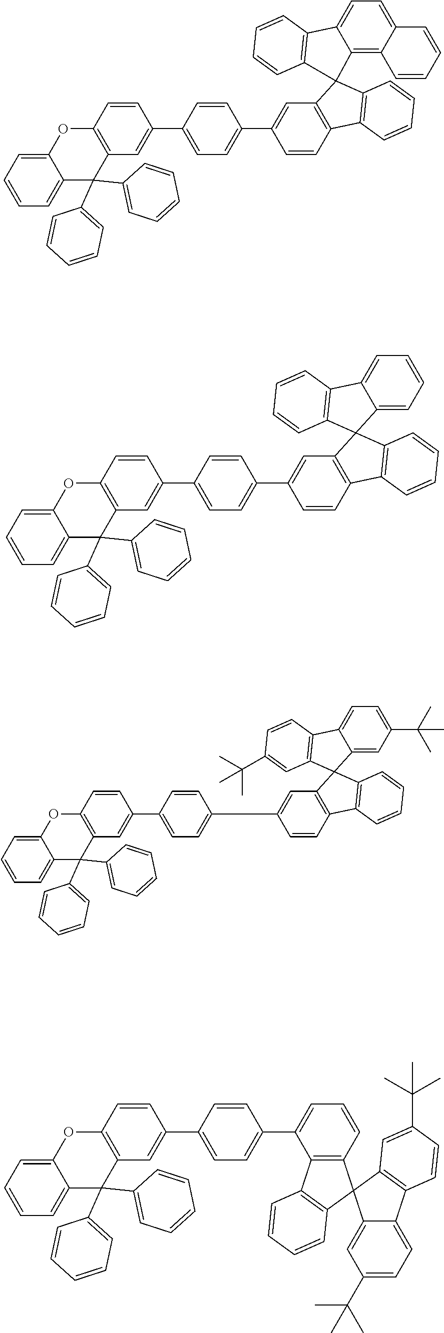 [SMILES: c1ccc(C2(c3ccccc3)c3ccccc3Oc3ccc(-c4ccc(-c5ccc6c(c5)C5(c7ccccc7-6)c6ccccc6-c6ccc7ccccc7c65)cc4)cc32)cc1 ; c1ccc(C2(c3ccccc3)c3ccccc3Oc3ccc(-c4ccc(-c5ccc6c(c5)C5(c7ccccc7-c7ccccc75)c5ccccc5-6)cc4)cc32)cc1 ; CC(C)(C)c1ccc2c(c1)C1(c3ccccc3-c3ccc(-c4ccc(-c5ccc6c(c5)C(c5ccccc5)(c5ccccc5)c5ccccc5O6)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4ccc5c(c4)C(c4ccccc4)(c4ccccc4)c4ccccc4O5)cc3)cccc21]

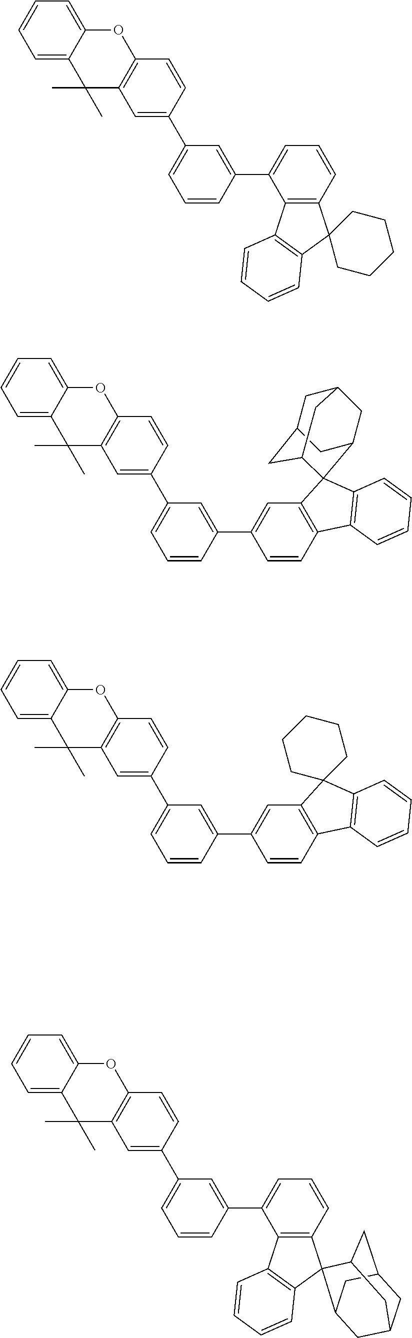 [SMILES: CC1(C)c2ccccc2Oc2ccc(-c3cccc(-c4cccc5c4-c4ccccc4C54CCCCC4)c3)cc21 ; CC1(C)c2ccccc2Oc2ccc(-c3cccc(-c4ccc5c(c4)C4(c6ccccc6-5)C5CC6CC(C5)CC4C6)c3)cc21 ; CC1(C)c2ccccc2Oc2ccc(-c3cccc(-c4ccc5c(c4)C4(CCCCC4)c4ccccc4-5)c3)cc21 ; CC1(C)c2ccccc2Oc2ccc(-c3cccc(-c4cccc5c4-c4ccccc4C54C5CC6CC(C5)CC4C6)c3)cc21]

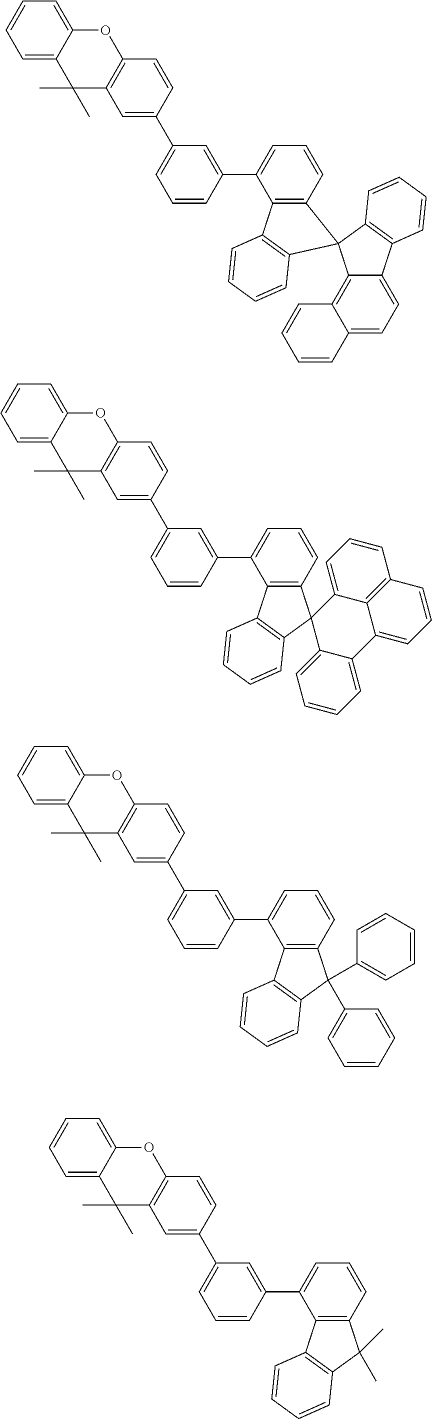 [SMILES: CC1(C)c2ccccc2Oc2ccc(-c3cccc(-c4cccc5c4-c4ccccc4C54c5ccccc5-c5ccc6ccccc6c54)c3)cc21 ; CC1(C)c2ccccc2Oc2ccc(-c3cccc(-c4cccc5c4-c4ccccc4C54c5ccccc5-c5cccc6cccc4c56)c3)cc21 ; CC1(C)c2ccccc2Oc2ccc(-c3cccc(-c4cccc5c4-c4ccccc4C5(c4ccccc4)c4ccccc4)c3)cc21 ; CC1(C)c2ccccc2Oc2ccc(-c3cccc(-c4cccc5c4-c4ccccc4C5(C)C)c3)cc21]

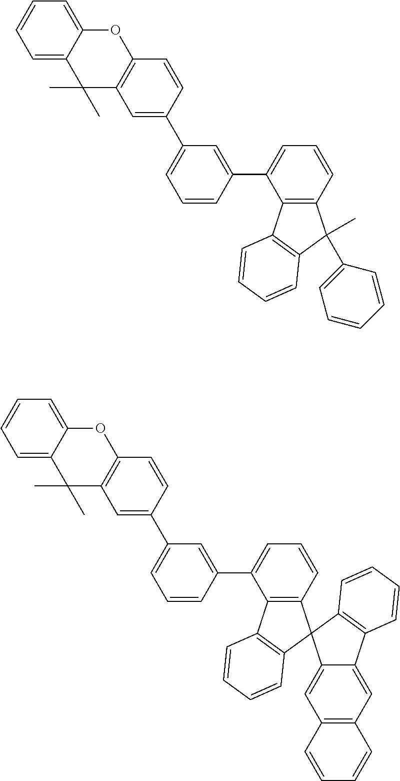 [SMILES: CC1(C)c2ccccc2Oc2ccc(-c3cccc(-c4cccc5c4-c4ccccc4C5(C)c4ccccc4)c3)cc21 ; CC1(C)c2ccccc2Oc2ccc(-c3cccc(-c4cccc5c4-c4ccccc4C54c5ccccc5-c5cc6ccccc6cc54)c3)cc21]

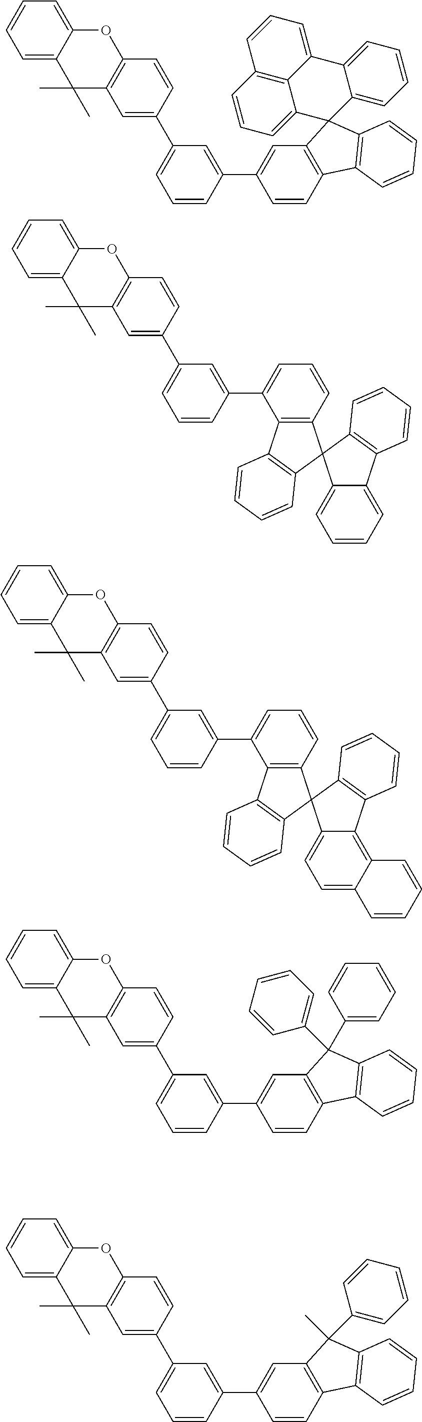

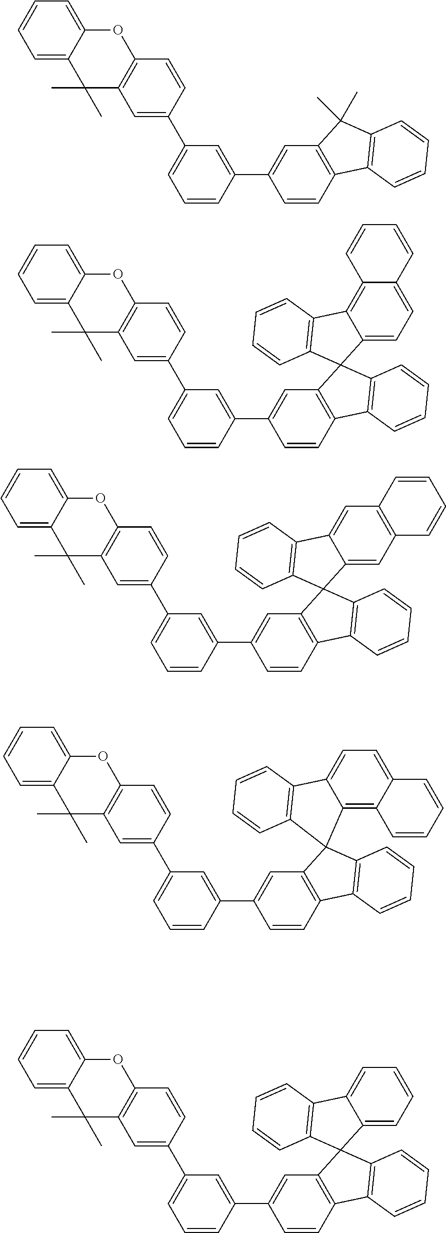

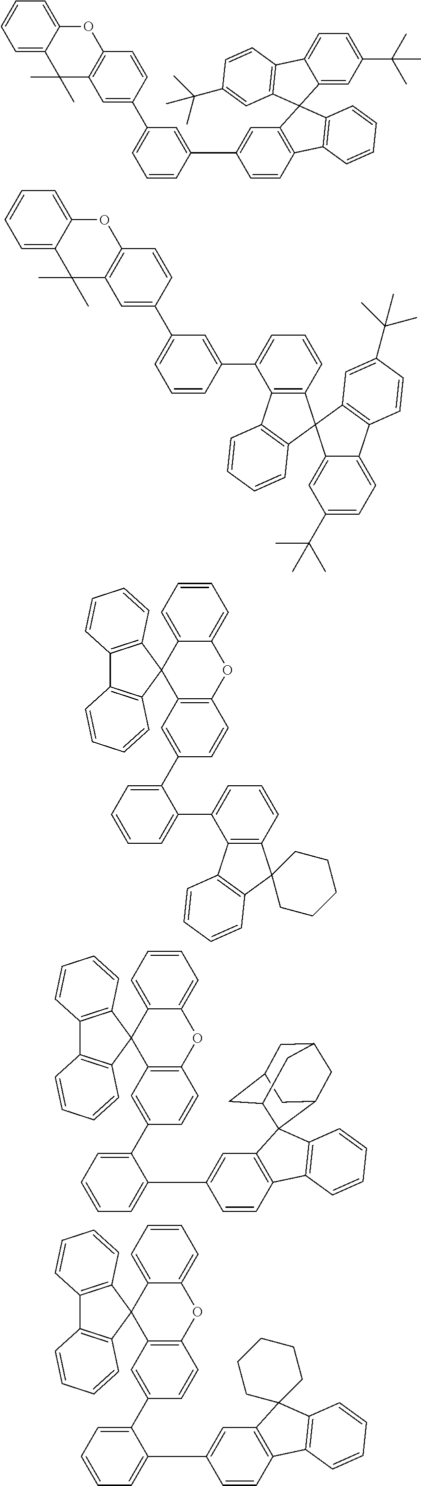

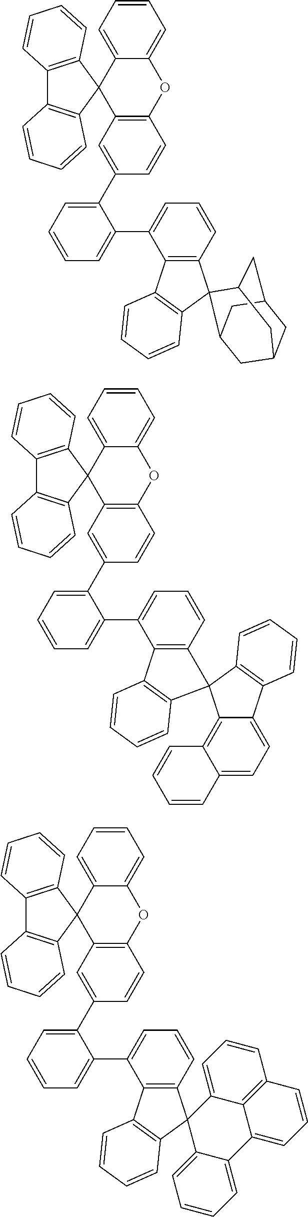

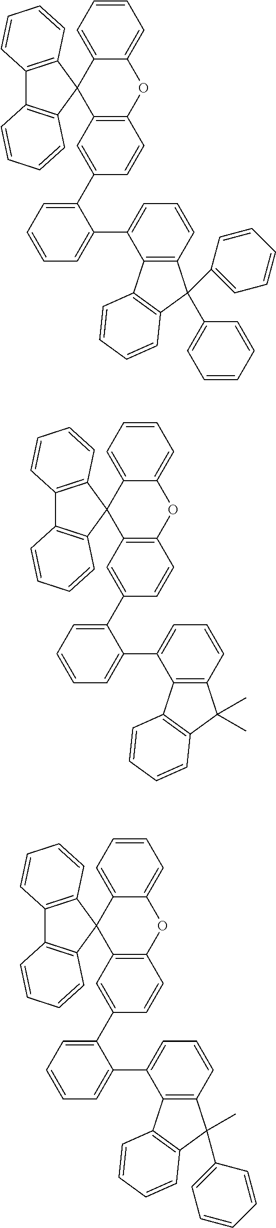 [SMILES: c1ccc(C2(c3ccccc3)c3ccccc3-c3c(-c4ccccc4-c4ccc5c(c4)C4(c6ccccc6O5)c5ccccc5-c5ccccc54)cccc32)cc1 ; CC1(C)c2ccccc2-c2c(-c3ccccc3-c3ccc4c(c3)C3(c5ccccc5O4)c4ccccc4-c4ccccc43)cccc21 ; CC1(c2ccccc2)c2ccccc2-c2c(-c3ccccc3-c3ccc4c(c3)C3(c5ccccc5O4)c4ccccc4-c4ccccc43)cccc21]

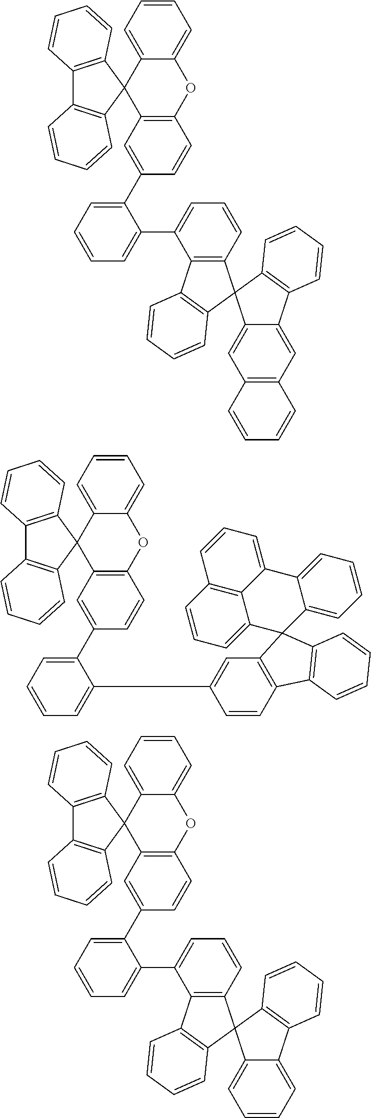

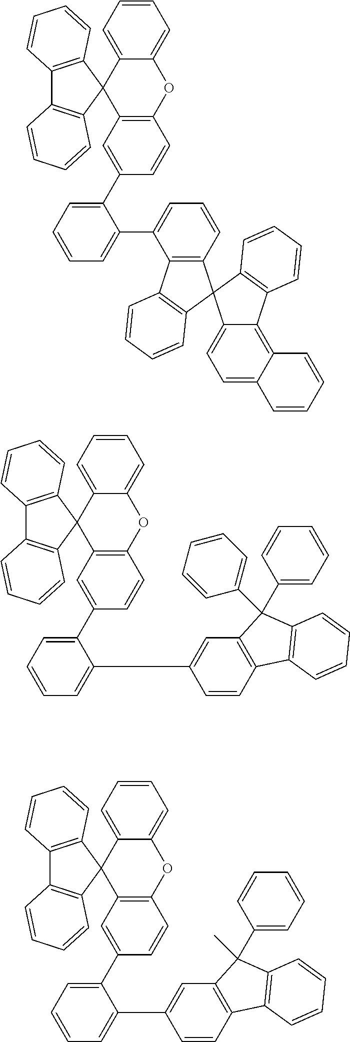 [SMILES: c1ccc2c(c1)Oc1ccc(-c3ccccc3-c3cccc4c3-c3ccccc3C43c4ccccc4-c4c3ccc3ccccc43)cc1C21c2ccccc2-c2ccccc21 ; c1ccc(C2(c3ccccc3)c3ccccc3-c3ccc(-c4ccccc4-c4ccc5c(c4)C4(c6ccccc6O5)c5ccccc5-c5ccccc54)cc32)cc1 ; CC1(c2ccccc2)c2ccccc2-c2ccc(-c3ccccc3-c3ccc4c(c3)C3(c5ccccc5O4)c4ccccc4-c4ccccc43)cc21]

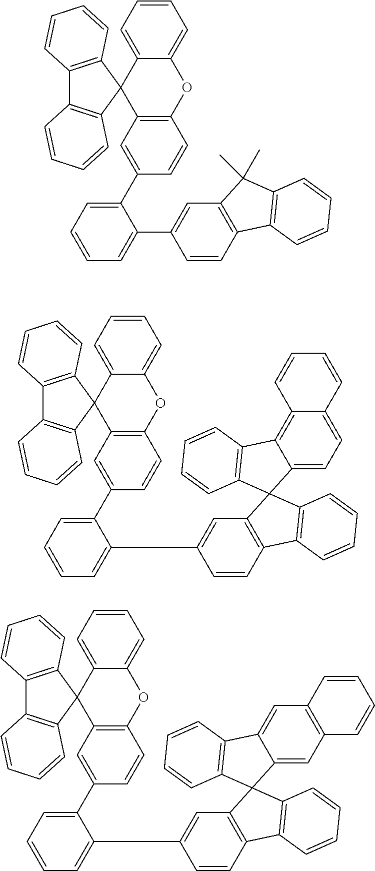

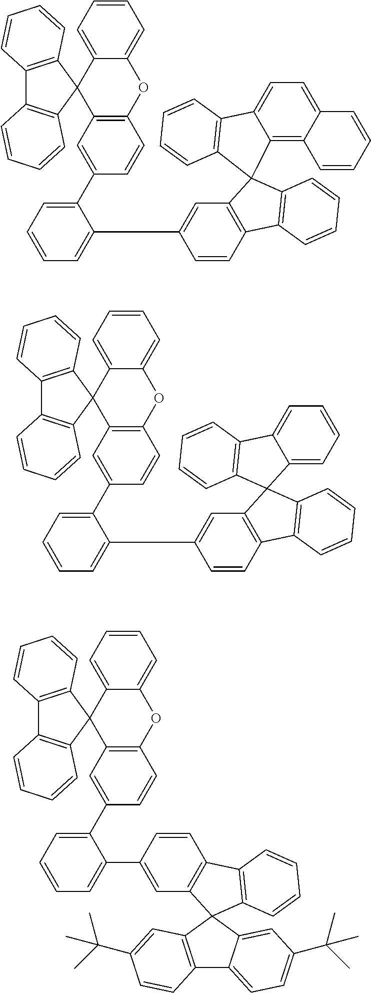 [SMILES: c1ccc2c(c1)Oc1ccc(-c3ccccc3-c3ccc4c(c3)C3(c5ccccc5-4)c4ccccc4-c4ccc5ccccc5c43)cc1C21c2ccccc2-c2ccccc21 ; c1ccc2c(c1)Oc1ccc(-c3ccccc3-c3ccc4c(c3)C3(c5ccccc5-c5ccccc53)c3ccccc3-4)cc1C21c2ccccc2-c2ccccc21 ; CC(C)(C)c1ccc2c(c1)C1(c3ccccc3-c3ccc(-c4ccccc4-c4ccc5c(c4)C4(c6ccccc6O5)c5ccccc5-c5ccccc54)cc31)c1cc(C(C)(C)C)ccc1-2]

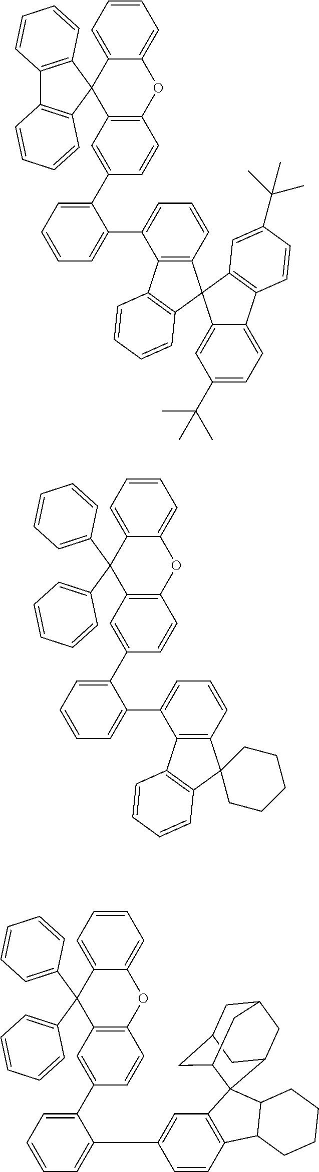

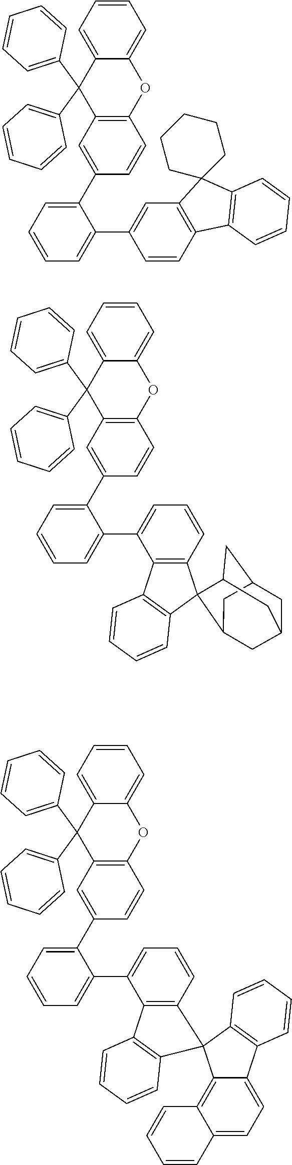

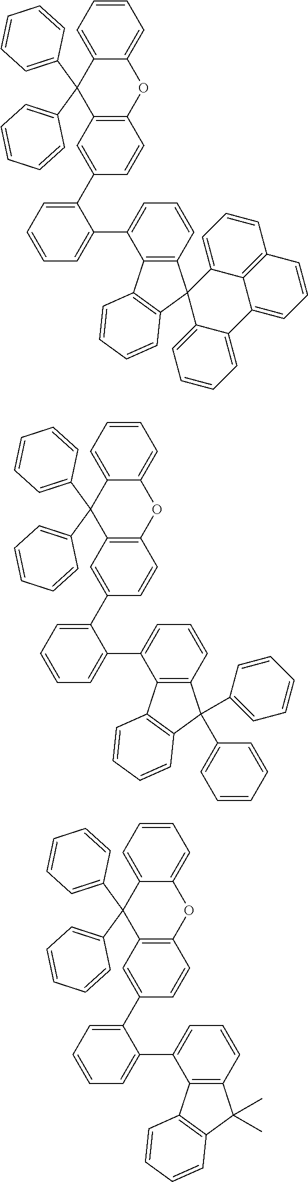

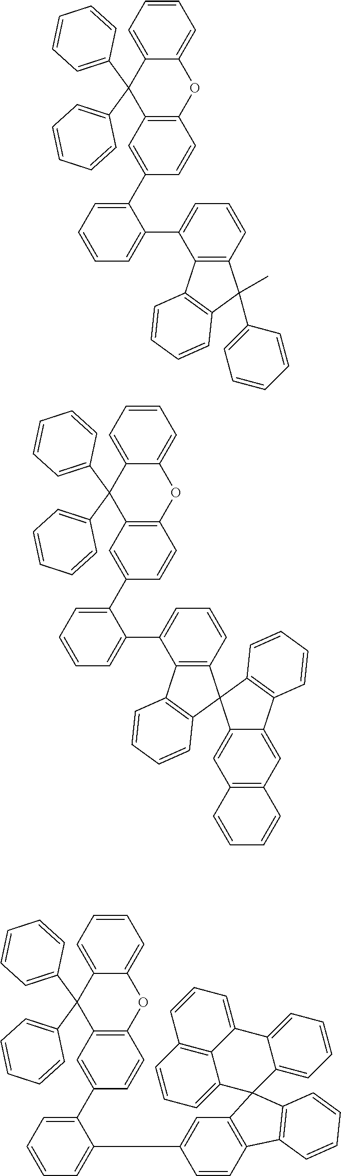 [SMILES: CC1(c2ccccc2)c2ccccc2-c2c(-c3ccccc3-c3ccc4c(c3)C(c3ccccc3)(c3ccccc3)c3ccccc3O4)cccc21 ; c1ccc(C2(c3ccccc3)c3ccccc3Oc3ccc(-c4ccccc4-c4cccc5c4-c4ccccc4C54c5ccccc5-c5cc6ccccc6cc54)cc32)cc1 ; c1ccc(C2(c3ccccc3)c3ccccc3Oc3ccc(-c4ccccc4-c4ccc5c(c4)C4(c6ccccc6-5)c5ccccc5-c5cccc6cccc4c56)cc32)cc1]

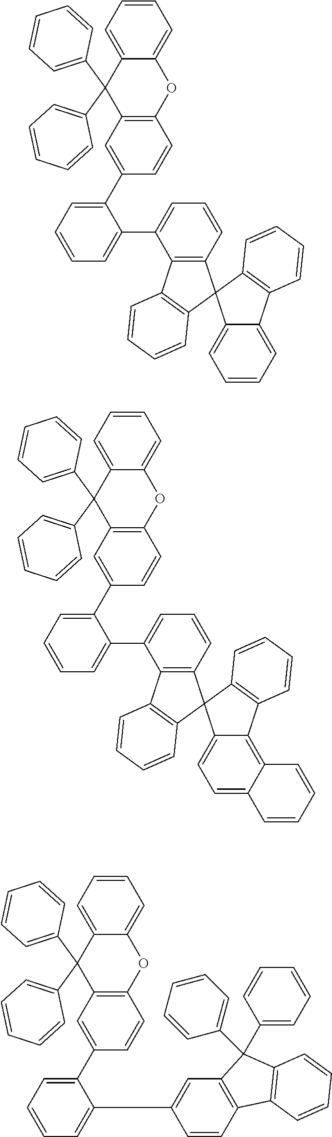 [SMILES: c1ccc(C2(c3ccccc3)c3ccccc3Oc3ccc(-c4ccccc4-c4cccc5c4-c4ccccc4C54c5ccccc5-c5ccccc54)cc32)cc1 ; c1ccc(C2(c3ccccc3)c3ccccc3Oc3ccc(-c4ccccc4-c4cccc5c4-c4ccccc4C54c5ccccc5-c5c4ccc4ccccc54)cc32)cc1 ; c1ccc(C2(c3ccccc3)c3ccccc3Oc3ccc(-c4ccccc4-c4ccc5c(c4)C(c4ccccc4)(c4ccccc4)c4ccccc4-5)cc32)cc1]

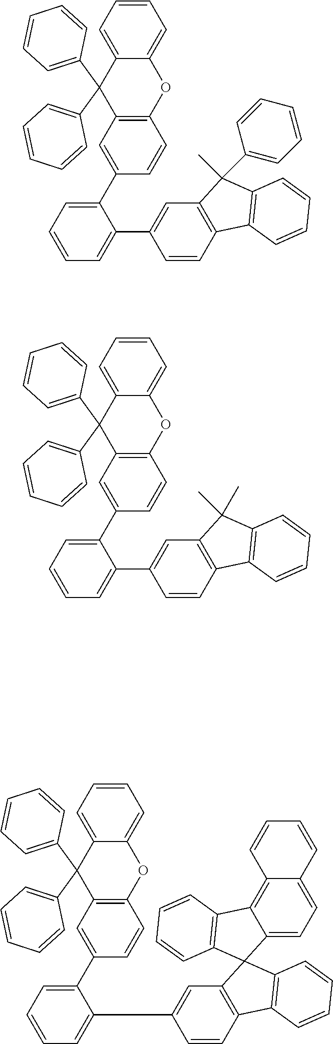 [SMILES: CC1(c2ccccc2)c2ccccc2-c2ccc(-c3ccccc3-c3ccc4c(c3)C(c3ccccc3)(c3ccccc3)c3ccccc3O4)cc21 ; CC1(C)c2ccccc2-c2ccc(-c3ccccc3-c3ccc4c(c3)C(c3ccccc3)(c3ccccc3)c3ccccc3O4)cc21 ; c1ccc(C2(c3ccccc3)c3ccccc3Oc3ccc(-c4ccccc4-c4ccc5c(c4)C4(c6ccccc6-5)c5ccccc5-c5c4ccc4ccccc54)cc32)cc1]

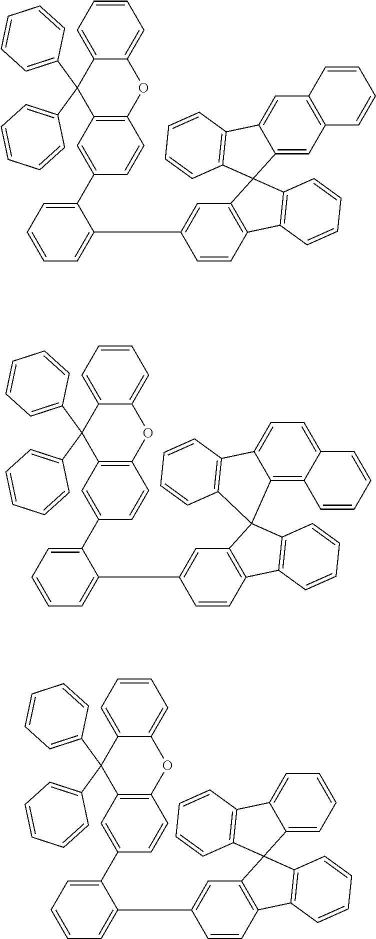 [SMILES: c1ccc(C2(c3ccccc3)c3ccccc3Oc3ccc(-c4ccccc4-c4ccc5c(c4)C4(c6ccccc6-5)c5ccccc5-c5cc6ccccc6cc54)cc32)cc1 ; c1ccc(C2(c3ccccc3)c3ccccc3Oc3ccc(-c4ccccc4-c4ccc5c(c4)C4(c6ccccc6-5)c5ccccc5-c5ccc6ccccc6c54)cc32)cc1 ; c1ccc(C2(c3ccccc3)c3ccccc3Oc3ccc(-c4ccccc4-c4ccc5c(c4)C4(c6ccccc6-c6ccccc64)c4ccccc4-5)cc32)cc1]

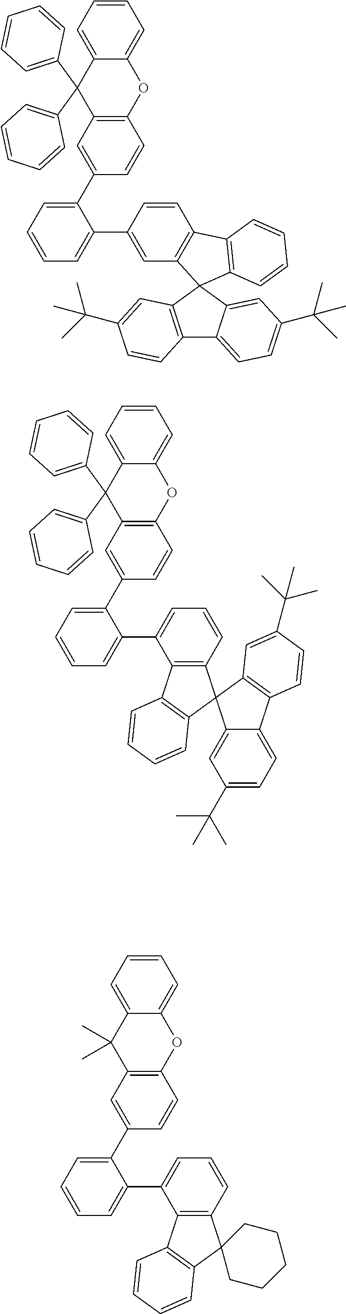

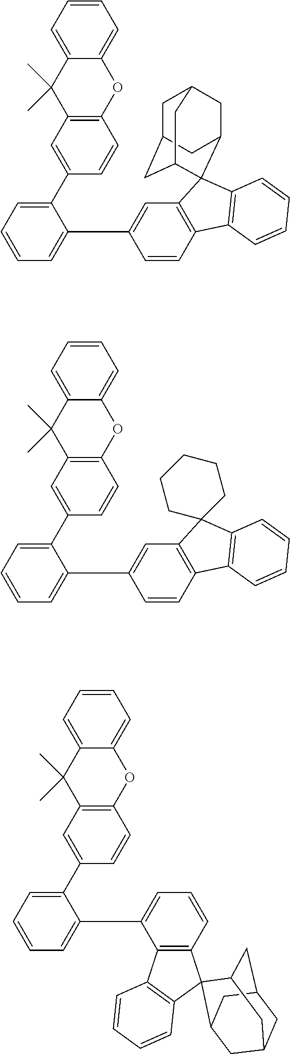 [SMILES: CC1(C)c2ccccc2Oc2ccc(-c3ccccc3-c3ccc4c(c3)C3(c5ccccc5-4)C4CC5CC(C4)CC3C5)cc21 ; CC1(C)c2ccccc2Oc2ccc(-c3ccccc3-c3ccc4c(c3)C3(CCCCC3)c3ccccc3-4)cc21 ; CC1(C)c2ccccc2Oc2ccc(-c3ccccc3-c3cccc4c3-c3ccccc3C43C4CC5CC(C4)CC3C5)cc21]

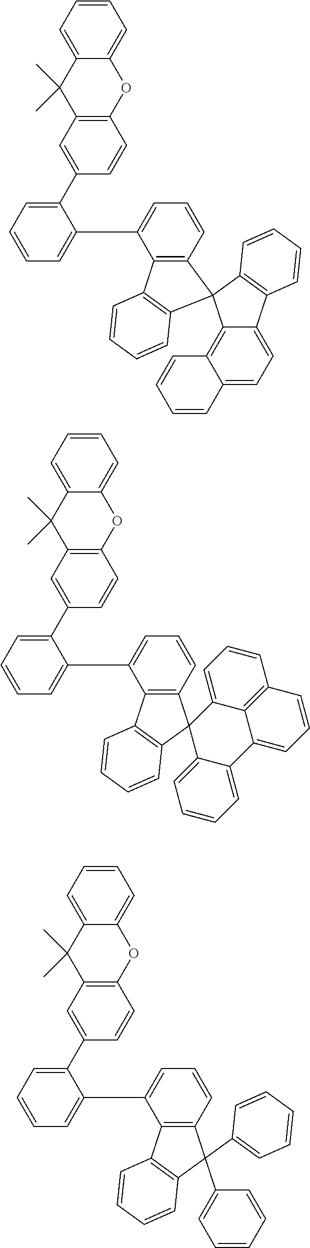 [SMILES: CC1(C)c2ccccc2Oc2ccc(-c3ccccc3-c3cccc4c3-c3ccccc3C43c4ccccc4-c4ccc5ccccc5c43)cc21 ; CC1(C)c2ccccc2Oc2ccc(-c3ccccc3-c3cccc4c3-c3ccccc3C43c4ccccc4-c4cccc5cccc3c45)cc21 ; CC1(C)c2ccccc2Oc2ccc(-c3ccccc3-c3cccc4c3-c3ccccc3C4(c3ccccc3)c3ccccc3)cc21]

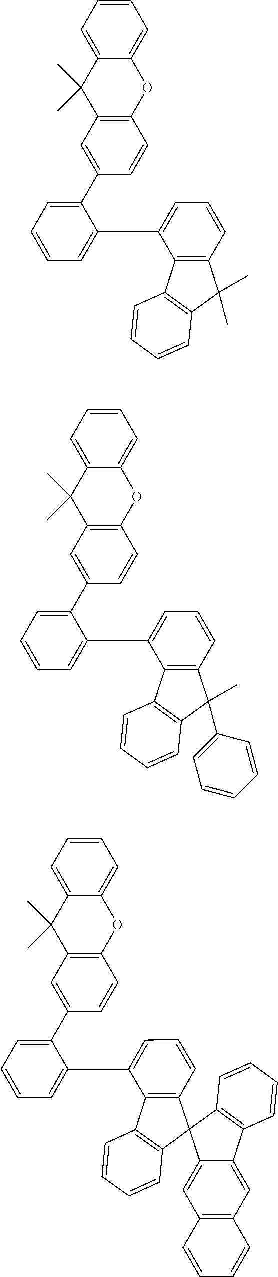 [SMILES: CC1(C)c2ccccc2Oc2ccc(-c3ccccc3-c3cccc4c3-c3ccccc3C4(C)C)cc21 ; CC1(C)c2ccccc2Oc2ccc(-c3ccccc3-c3cccc4c3-c3ccccc3C4(C)c3ccccc3)cc21 ; CC1(C)c2ccccc2Oc2ccc(-c3ccccc3-c3cccc4c3-c3ccccc3C43c4ccccc4-c4cc5ccccc5cc43)cc21]

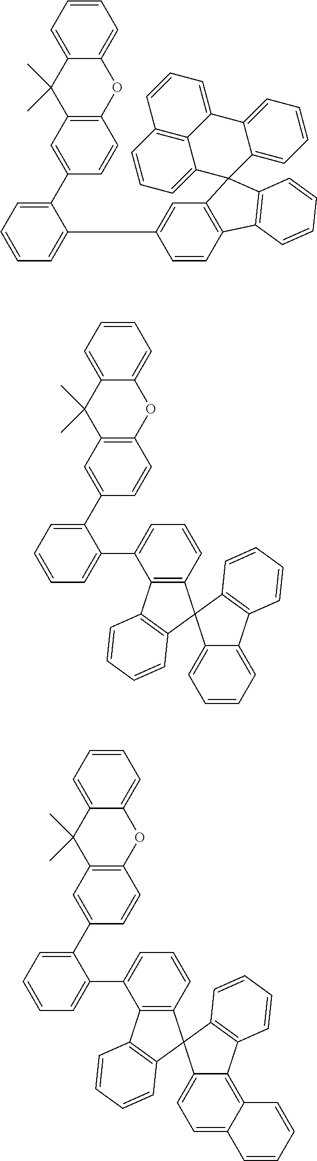 [SMILES: CC1(C)c2ccccc2Oc2ccc(-c3ccccc3-c3ccc4c(c3)C3(c5ccccc5-4)c4ccccc4-c4cccc5cccc3c45)cc21 ; CC1(C)c2ccccc2Oc2ccc(-c3ccccc3-c3cccc4c3-c3ccccc3C43c4ccccc4-c4ccccc43)cc21 ; CC1(C)c2ccccc2Oc2ccc(-c3ccccc3-c3cccc4c3-c3ccccc3C43c4ccccc4-c4c3ccc3ccccc43)cc21]

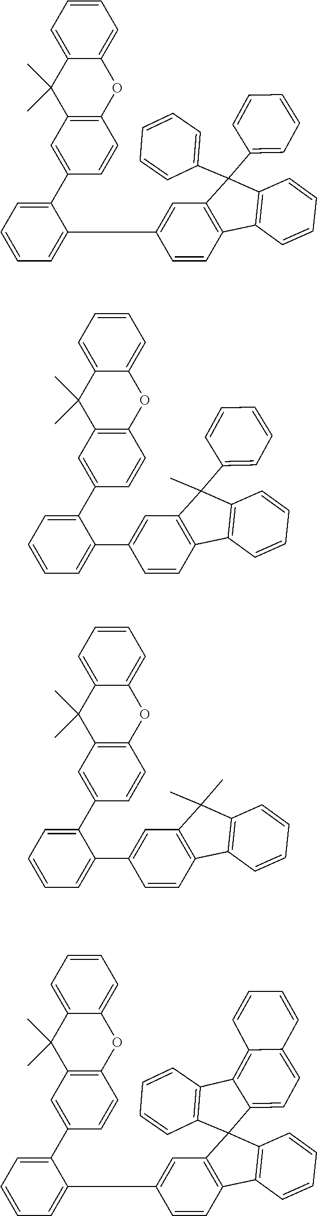 [SMILES: CC1(C)c2ccccc2Oc2ccc(-c3ccccc3-c3ccc4c(c3)C(c3ccccc3)(c3ccccc3)c3ccccc3-4)cc21 ; CC1(C)c2ccccc2Oc2ccc(-c3ccccc3-c3ccc4c(c3)C(C)(c3ccccc3)c3ccccc3-4)cc21 ; CC1(C)c2ccccc2Oc2ccc(-c3ccccc3-c3ccc4c(c3)C(C)(C)c3ccccc3-4)cc21 ; CC1(C)c2ccccc2Oc2ccc(-c3ccccc3-c3ccc4c(c3)C3(c5ccccc5-4)c4ccccc4-c4c3ccc3ccccc43)cc21]

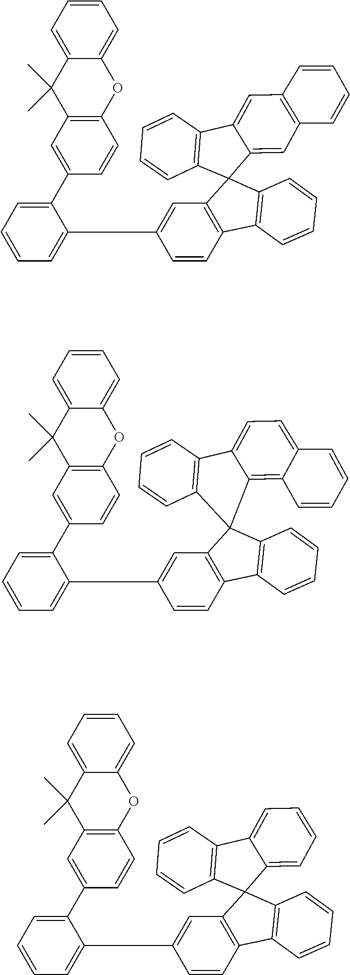 [SMILES: CC1(C)c2ccccc2Oc2ccc(-c3ccccc3-c3ccc4c(c3)C3(c5ccccc5-4)c4ccccc4-c4cc5ccccc5cc43)cc21 ; CC1(C)c2ccccc2Oc2ccc(-c3ccccc3-c3ccc4c(c3)C3(c5ccccc5-4)c4ccccc4-c4ccc5ccccc5c43)cc21 ; CC1(C)c2ccccc2Oc2ccc(-c3ccccc3-c3ccc4c(c3)C3(c5ccccc5-c5ccccc53)c3ccccc3-4)cc21]

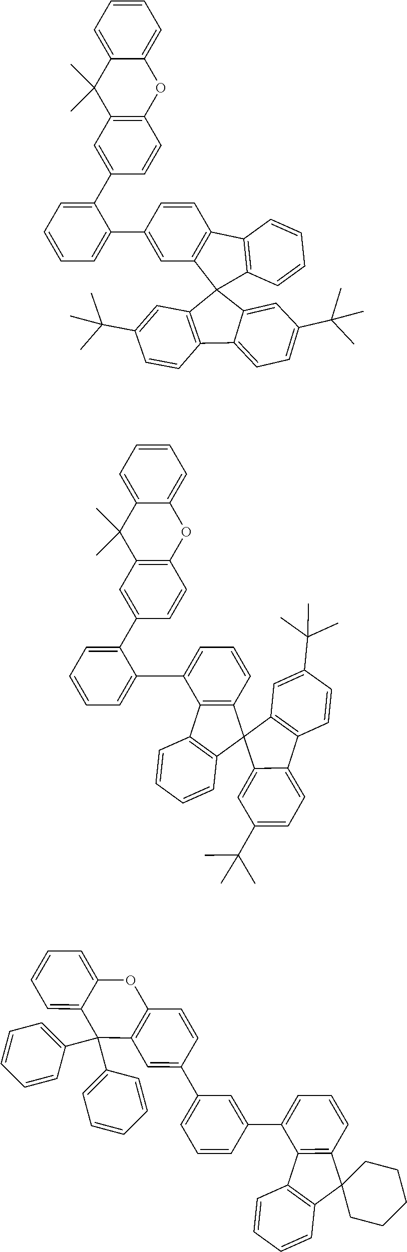 [SMILES: CC(C)(C)c1ccc2c(c1)C1(c3ccccc3-c3ccc(-c4ccccc4-c4ccc5c(c4)C(C)(C)c4ccccc4O5)cc31)c1cc(C(C)(C)C)ccc1-2 ; CC(C)(C)c1ccc2c(c1)C1(c3cc(C(C)(C)C)ccc3-2)c2ccccc2-c2c(-c3ccccc3-c3ccc4c(c3)C(C)(C)c3ccccc3O4)cccc21 ; c1ccc(C2(c3ccccc3)c3ccccc3Oc3ccc(-c4cccc(-c5cccc6c5-c5ccccc5C65CCCCC5)c4)cc32)cc1]

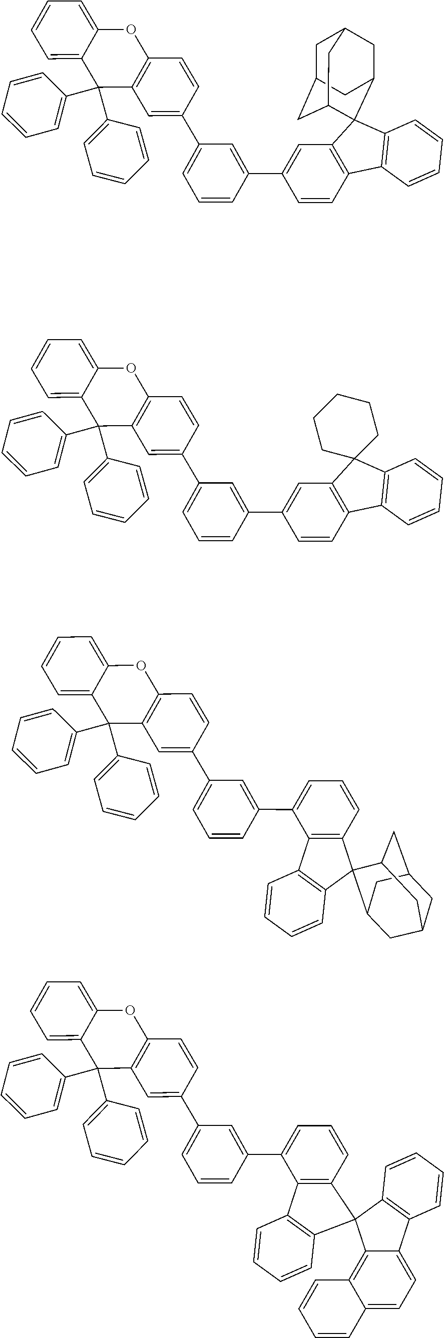 [SMILES: c1ccc(C2(c3ccccc3)c3ccccc3Oc3ccc(-c4cccc(-c5ccc6c(c5)C5(c7ccccc7-6)C6CC7CC(C6)CC5C7)c4)cc32)cc1 ; c1ccc(C2(c3ccccc3)c3ccccc3Oc3ccc(-c4cccc(-c5ccc6c(c5)C5(CCCCC5)c5ccccc5-6)c4)cc32)cc1 ; c1ccc(C2(c3ccccc3)c3ccccc3Oc3ccc(-c4cccc(-c5cccc6c5-c5ccccc5C65C6CC7CC(C6)CC5C7)c4)cc32)cc1 ; c1ccc(C2(c3ccccc3)c3ccccc3Oc3ccc(-c4cccc(-c5cccc6c5-c5ccccc5C65c6ccccc6-c6ccc7ccccc7c65)c4)cc32)cc1]

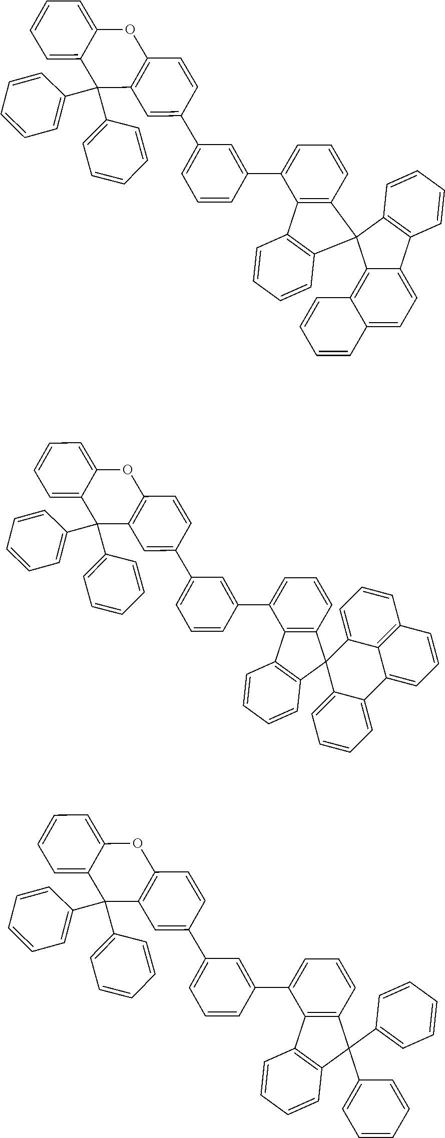 [SMILES: c1ccc(C2(c3ccccc3)c3ccccc3Oc3ccc(-c4cccc(-c5cccc6c5-c5ccccc5C65c6ccccc6-c6ccc7ccccc7c65)c4)cc32)cc1 ; c1ccc(C2(c3ccccc3)c3ccccc3Oc3ccc(-c4cccc(-c5cccc6c5-c5ccccc5C65c6ccccc6-c6cccc7cccc5c67)c4)cc32)cc1 ; c1ccc(C2(c3ccccc3)c3ccccc3Oc3ccc(-c4cccc(-c5cccc6c5-c5ccccc5C6(c5ccccc5)c5ccccc5)c4)cc32)cc1]

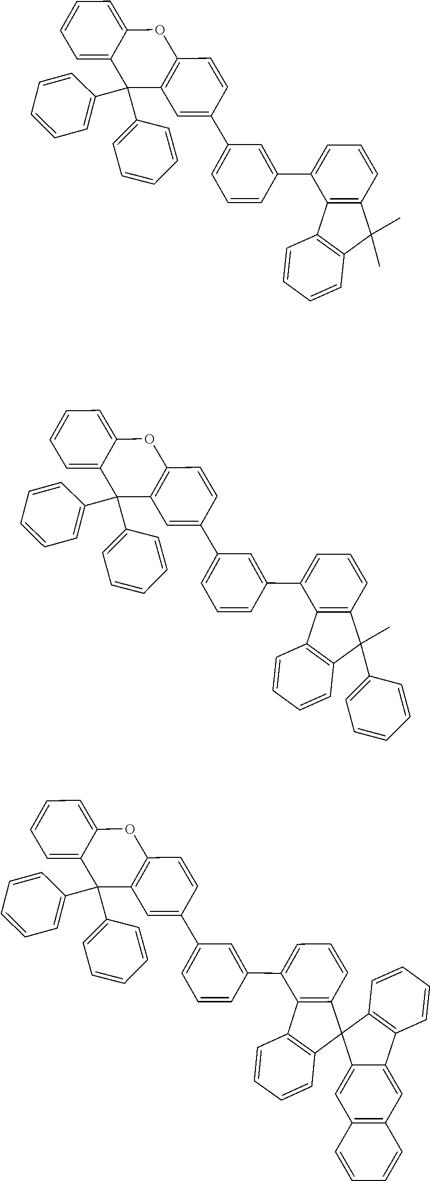 [SMILES: CC1(C)c2ccccc2-c2c(-c3cccc(-c4ccc5c(c4)C(c4ccccc4)(c4ccccc4)c4ccccc4O5)c3)cccc21 ; CC1(c2ccccc2)c2ccccc2-c2c(-c3cccc(-c4ccc5c(c4)C(c4ccccc4)(c4ccccc4)c4ccccc4O5)c3)cccc21 ; c1ccc(C2(c3ccccc3)c3ccccc3Oc3ccc(-c4cccc(-c5cccc6c5-c5ccccc5C65c6ccccc6-c6cc7ccccc7cc65)c4)cc32)cc1]

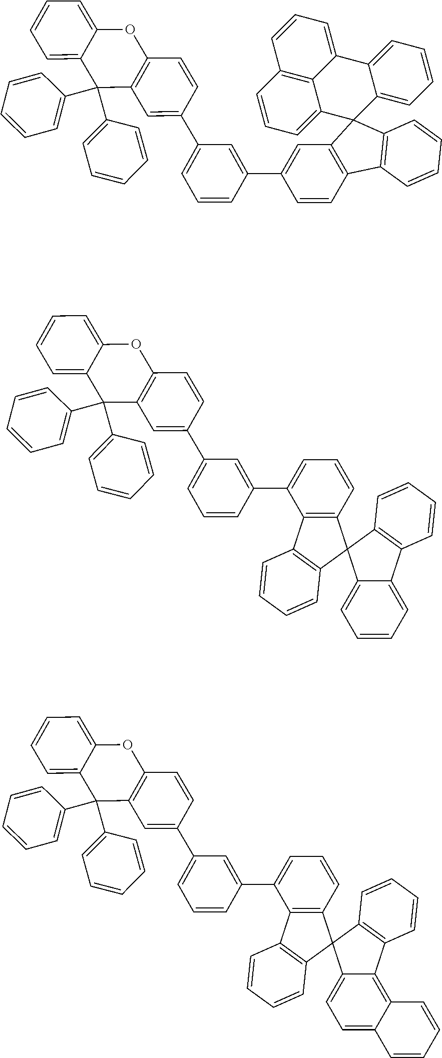 [SMILES: c1ccc(C2(c3ccccc3)c3ccccc3Oc3ccc(-c4cccc(-c5ccc6c(c5)C5(c7ccccc7-6)c6ccccc6-c6cccc7cccc5c67)c4)cc32)cc1 ; c1ccc(C2(c3ccccc3)c3ccccc3Oc3ccc(-c4cccc(-c5cccc6c5-c5ccccc5C65c6ccccc6-c6ccccc65)c4)cc32)cc1 ; c1ccc(C2(c3ccccc3)c3ccccc3Oc3ccc(-c4cccc(-c5cccc6c5-c5ccccc5C65c6ccccc6-c6c5ccc5ccccc65)c4)cc32)cc1]

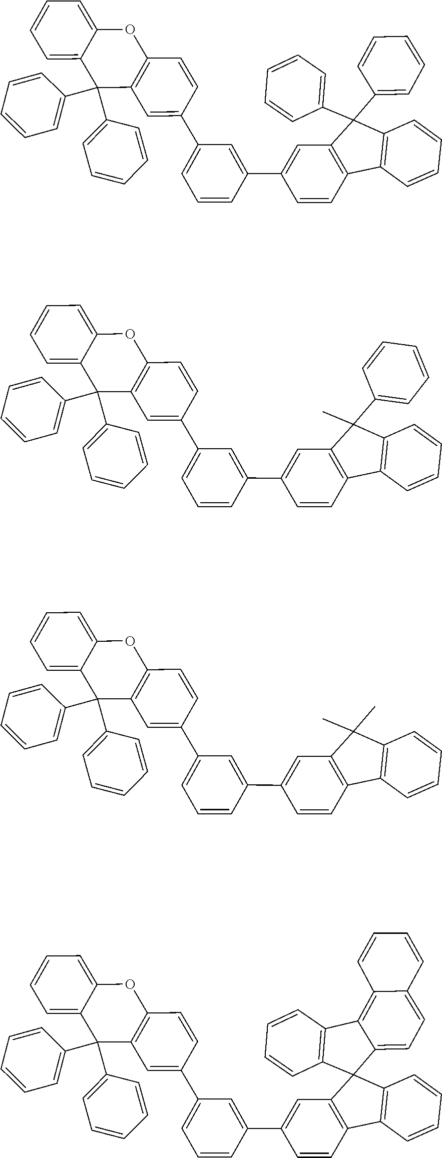 [SMILES: c1ccc(C2(c3ccccc3)c3ccccc3Oc3ccc(-c4cccc(-c5ccc6c(c5)C(c5ccccc5)(c5ccccc5)c5ccccc5-6)c4)cc32)cc1 ; CC1(c2ccccc2)c2ccccc2-c2ccc(-c3cccc(-c4ccc5c(c4)C(c4ccccc4)(c4ccccc4)c4ccccc4O5)c3)cc21 ; CC1(C)c2ccccc2-c2ccc(-c3cccc(-c4ccc5c(c4)C(c4ccccc4)(c4ccccc4)c4ccccc4O5)c3)cc21 ; c1ccc(C2(c3ccccc3)c3ccccc3Oc3ccc(-c4cccc(-c5ccc6c(c5)C5(c7ccccc7-6)c6ccccc6-c6c5ccc5ccccc65)c4)cc32)cc1]

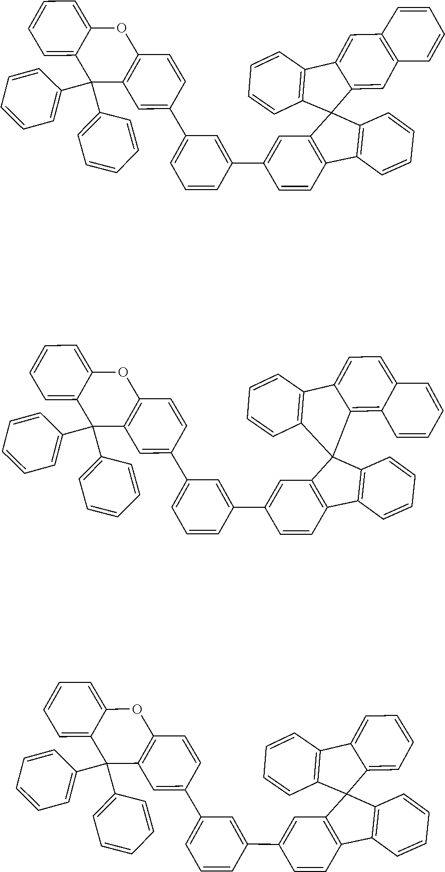 [SMILES: c1ccc(C2(c3ccccc3)c3ccccc3Oc3ccc(-c4cccc(-c5ccc6c(c5)C5(c7ccccc7-6)c6ccccc6-c6cc7ccccc7cc65)c4)cc32)cc1 ; c1ccc(C2(c3ccccc3)c3ccccc3Oc3ccc(-c4cccc(-c5ccc6c(c5)C5(c7ccccc7-6)c6ccccc6-c6ccc7ccccc7c65)c4)cc32)cc1 ; c1ccc(C2(c3ccccc3)c3ccccc3Oc3ccc(-c4cccc(-c5ccc6c(c5)C5(c7ccccc7-c7ccccc75)c5ccccc5-6)c4)cc32)cc1]

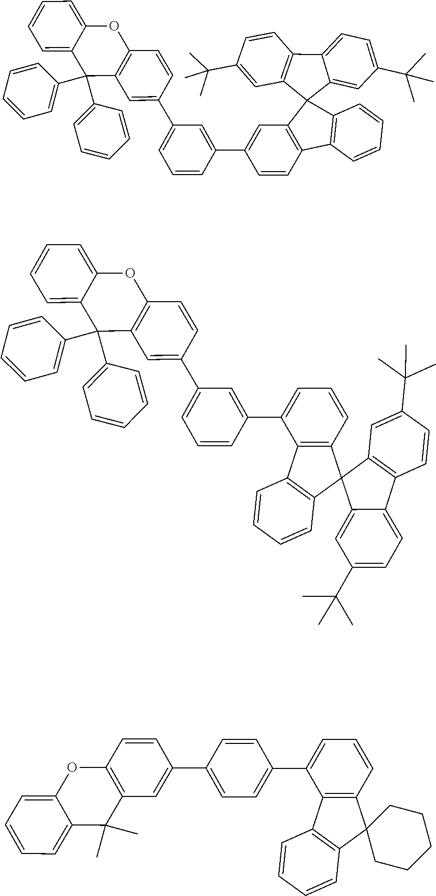 [SMILES: CC(C)(C)c1ccc2c(c1)C1(c3ccccc3-c3ccc(-c4cccc(-c5ccc6c(c5)C(c5ccccc5)(c5ccccc5)c5ccccc5O6)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4ccc5c(c4)C(c4ccccc4)(c4ccccc4)c4ccccc4O5)c3)cccc21 ; CC1(C)c2ccccc2Oc2ccc(-c3ccc(-c4cccc5c4-c4ccccc4C54CCCCC4)cc3)cc21]

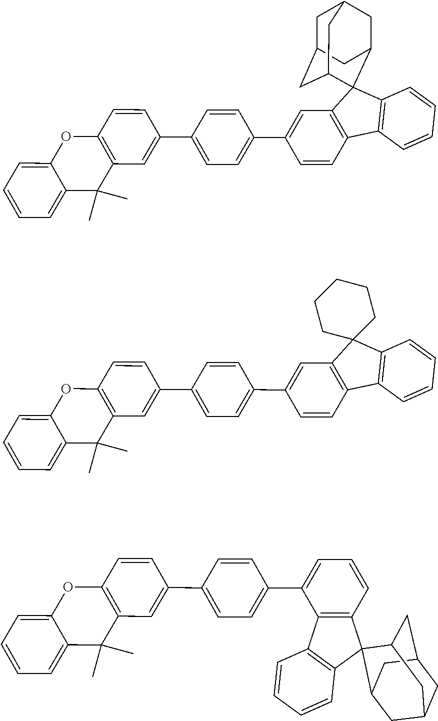 [SMILES: CC1(C)c2ccccc2Oc2ccc(-c3ccc(-c4ccc5c(c4)C4(c6ccccc6-5)C5CC6CC(C5)CC4C6)cc3)cc21 ; CC1(C)c2ccccc2Oc2ccc(-c3ccc(-c4ccc5c(c4)C4(CCCCC4)c4ccccc4-5)cc3)cc21 ; CC1(C)c2ccccc2Oc2ccc(-c3ccc(-c4cccc5c4-c4ccccc4C54C5CC6CC(C5)CC4C6)cc3)cc21]

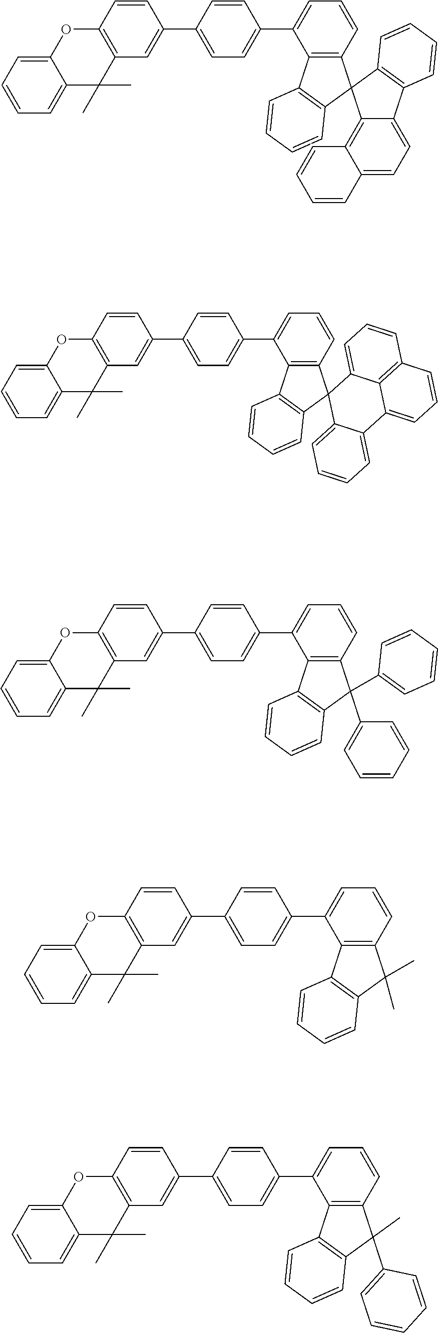 [SMILES: CC1(C)c2ccccc2Oc2ccc(-c3ccc(-c4cccc5c4-c4ccccc4C54c5ccccc5-c5ccc6ccccc6c54)cc3)cc21 ; CC1(C)c2ccccc2Oc2ccc(-c3ccc(-c4cccc5c4-c4ccccc4C54c5ccccc5-c5cccc6cccc4c56)cc3)cc21 ; CC1(C)c2ccccc2Oc2ccc(-c3ccc(-c4cccc5c4-c4ccccc4C5(c4ccccc4)c4ccccc4)cc3)cc21 ; CC1(C)c2ccccc2Oc2ccc(-c3ccc(-c4cccc5c4-c4ccccc4C5(C)C)cc3)cc21 ; CC1(C)c2ccccc2Oc2ccc(-c3ccc(-c4cccc5c4-c4ccccc4C5(C)c4ccccc4)cc3)cc21]

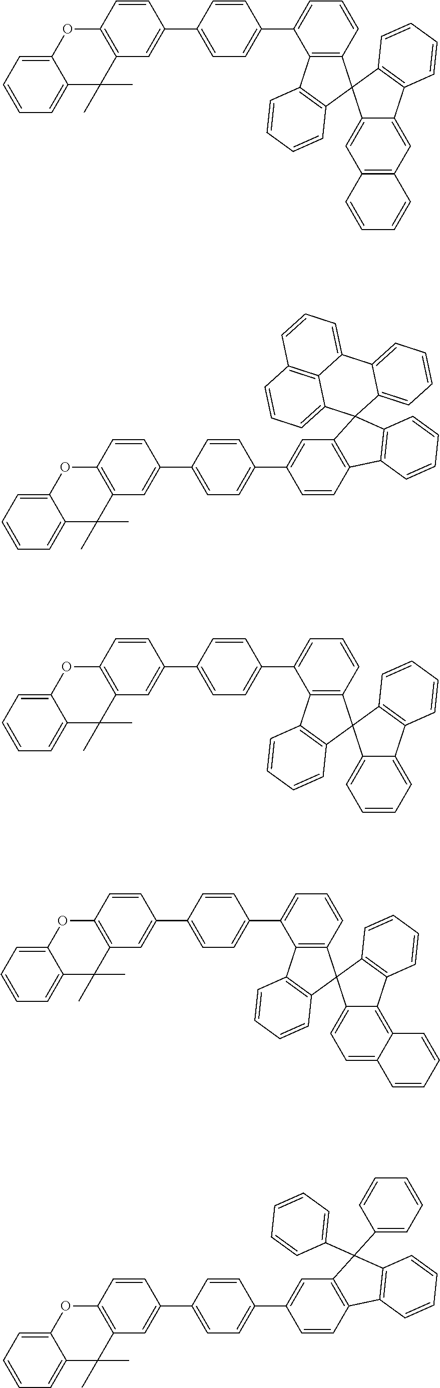 [SMILES: CC1(C)c2ccccc2Oc2ccc(-c3ccc(-c4cccc5c4-c4ccccc4C54c5ccccc5-c5cc6ccccc6cc54)cc3)cc21 ; CC1(C)c2ccccc2Oc2ccc(-c3ccc(-c4ccc5c(c4)C4(c6ccccc6-5)c5ccccc5-c5cccc6cccc4c56)cc3)cc21 ; CC1(C)c2ccccc2Oc2ccc(-c3ccc(-c4cccc5c4-c4ccccc4C54c5ccccc5-c5ccccc54)cc3)cc21 ; CC1(C)c2ccccc2Oc2ccc(-c3ccc(-c4cccc5c4-c4ccccc4C54c5ccccc5-c5c4ccc4ccccc54)cc3)cc21 ; CC1(C)c2ccccc2Oc2ccc(-c3ccc(-c4ccc5c(c4)C(c4ccccc4)(c4ccccc4)c4ccccc4-5)cc3)cc21]

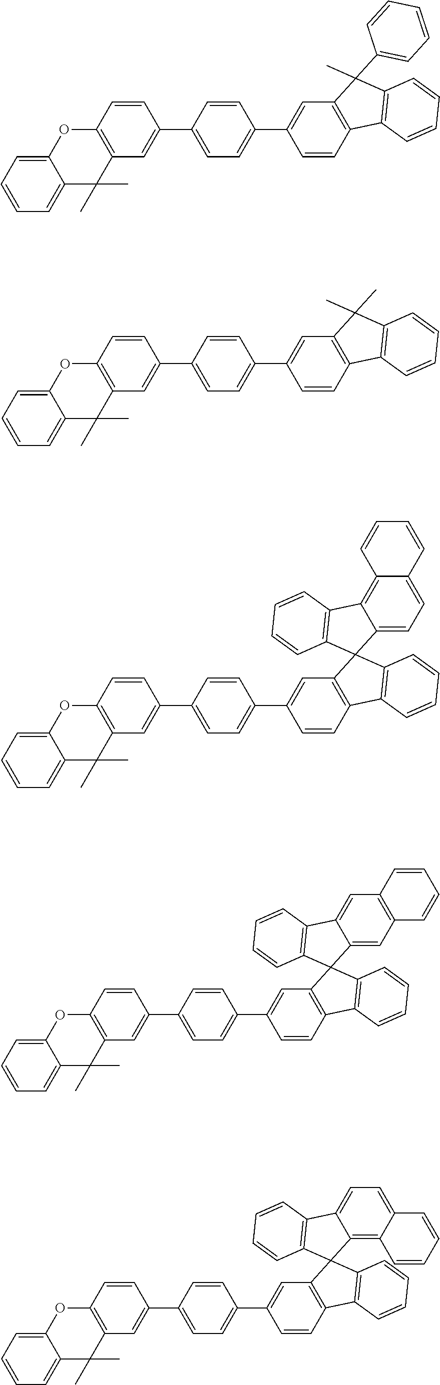 [SMILES: CC1(C)c2ccccc2Oc2ccc(-c3ccc(-c4ccc5c(c4)C(C)(c4ccccc4)c4ccccc4-5)cc3)cc21 ; CC1(C)c2ccccc2Oc2ccc(-c3ccc(-c4ccc5c(c4)C(C)(C)c4ccccc4-5)cc3)cc21 ; CC1(C)c2ccccc2Oc2ccc(-c3ccc(-c4ccc5c(c4)C4(c6ccccc6-5)c5ccccc5-c5c4ccc4ccccc54)cc3)cc21 ; CC1(C)c2ccccc2Oc2ccc(-c3ccc(-c4ccc5c(c4)C4(c6ccccc6-5)c5ccccc5-c5cc6ccccc6cc54)cc3)cc21 ; CC1(C)c2ccccc2Oc2ccc(-c3ccc(-c4ccc5c(c4)C4(c6ccccc6-5)c5ccccc5-c5ccc6ccccc6c54)cc3)cc21]

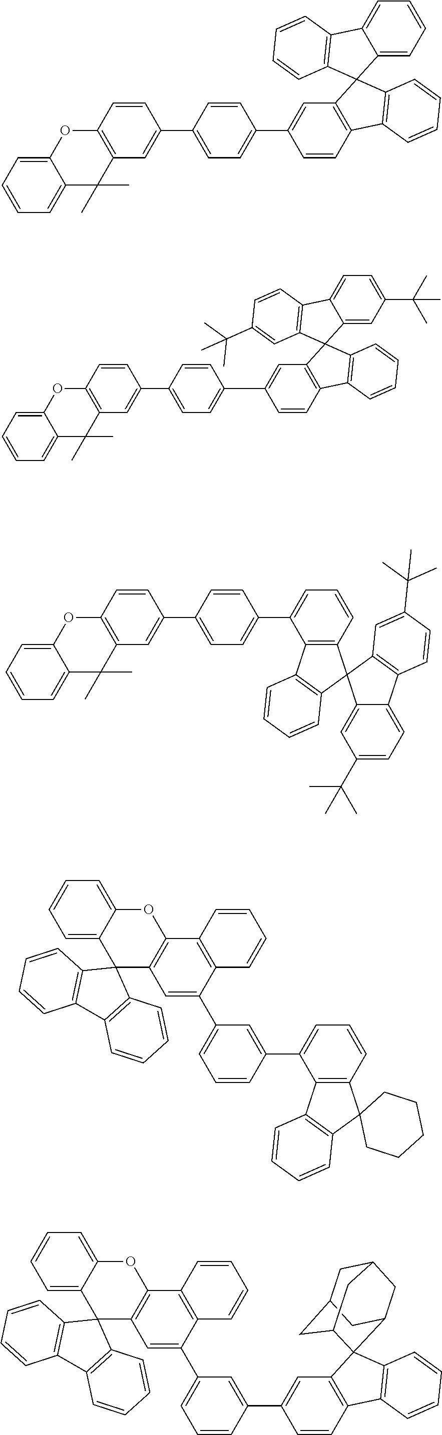 [SMILES: CC1(C)c2ccccc2Oc2ccc(-c3ccc(-c4ccc5c(c4)C4(c6ccccc6-c6ccccc64)c4ccccc4-5)cc3)cc21 ; CC(C)(C)c1ccc2c(c1)C1(c3ccccc3-c3ccc(-c4ccc(-c5ccc6c(c5)C(C)(C)c5ccccc5O6)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4ccc5c(c4)C(C)(C)c4ccccc4O5)cc3)cccc21 ; c1cc(-c2cccc3c2-c2ccccc2C32CCCCC2)cc(-c2cc3c(c4ccccc24)Oc2ccccc2C32c3ccccc3-c3ccccc32)c1 ; c1cc(-c2ccc3c(c2)C2(c4ccccc4-3)C3CC4CC(C3)CC2C4)cc(-c2cc3c(c4ccccc24)Oc2ccccc2C32c3ccccc3-c3ccccc32)c1]

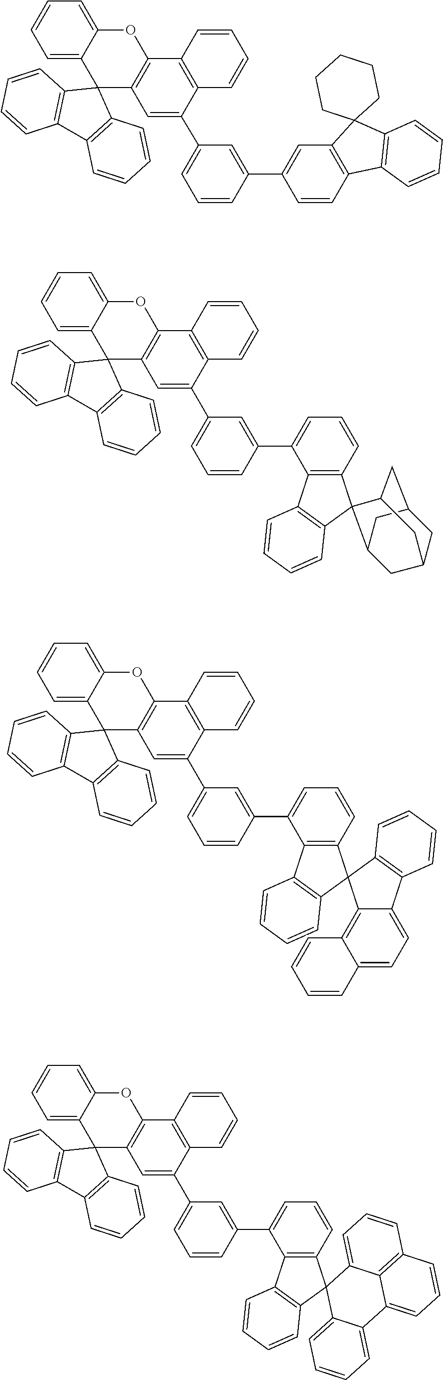 [SMILES: c1cc(-c2ccc3c(c2)C2(CCCCC2)c2ccccc2-3)cc(-c2cc3c(c4ccccc24)Oc2ccccc2C32c3ccccc3-c3ccccc32)c1 ; c1cc(-c2cccc3c2-c2ccccc2C32C3CC4CC(C3)CC2C4)cc(-c2cc3c(c4ccccc24)Oc2ccccc2C32c3ccccc3-c3ccccc32)c1 ; c1cc(-c2cccc3c2-c2ccccc2C32c3ccccc3-c3ccc4ccccc4c32)cc(-c2cc3c(c4ccccc24)Oc2ccccc2C32c3ccccc3-c3ccccc32)c1 ; c1cc(-c2cccc3c2-c2ccccc2C32c3ccccc3-c3cccc4cccc2c34)cc(-c2cc3c(c4ccccc24)Oc2ccccc2C32c3ccccc3-c3ccccc32)c1]

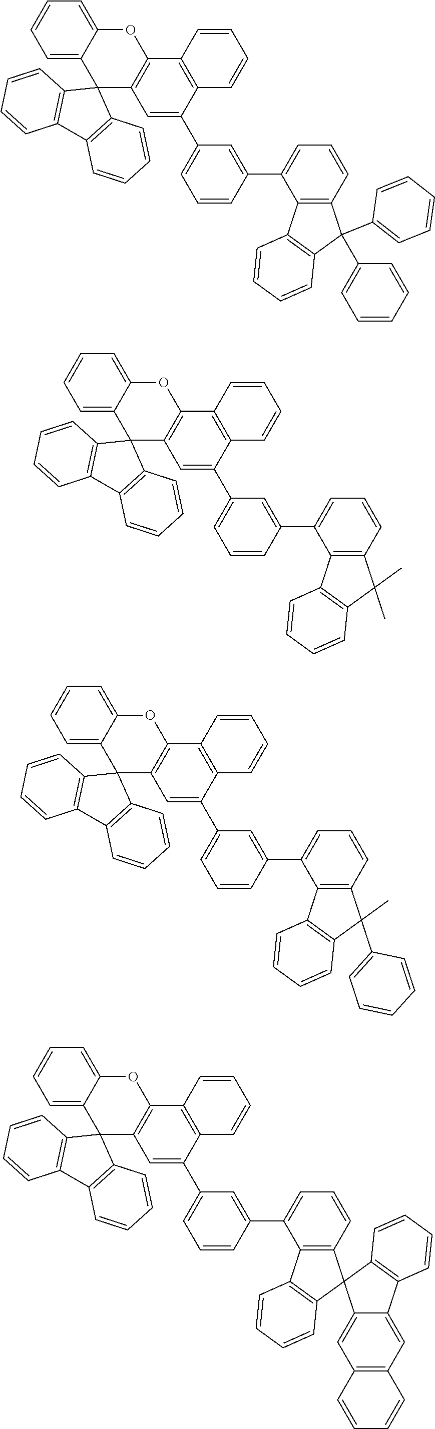 [SMILES: c1ccc(C2(c3ccccc3)c3ccccc3-c3c(-c4cccc(-c5cc6c(c7ccccc57)Oc5ccccc5C65c6ccccc6-c6ccccc65)c4)cccc32)cc1 ; CC1(C)c2ccccc2-c2c(-c3cccc(-c4cc5c(c6ccccc46)Oc4ccccc4C54c5ccccc5-c5ccccc54)c3)cccc21 ; CC1(c2ccccc2)c2ccccc2-c2c(-c3cccc(-c4cc5c(c6ccccc46)Oc4ccccc4C54c5ccccc5-c5ccccc54)c3)cccc21 ; c1cc(-c2cccc3c2-c2ccccc2C32c3ccccc3-c3cc4ccccc4cc32)cc(-c2cc3c(c4ccccc24)Oc2ccccc2C32c3ccccc3-c3ccccc32)c1]

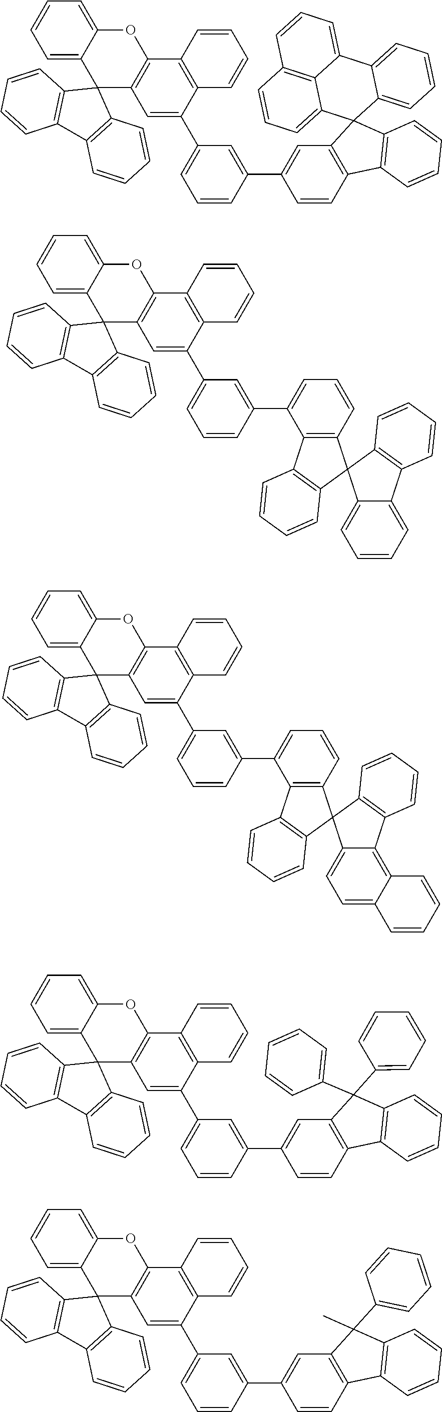

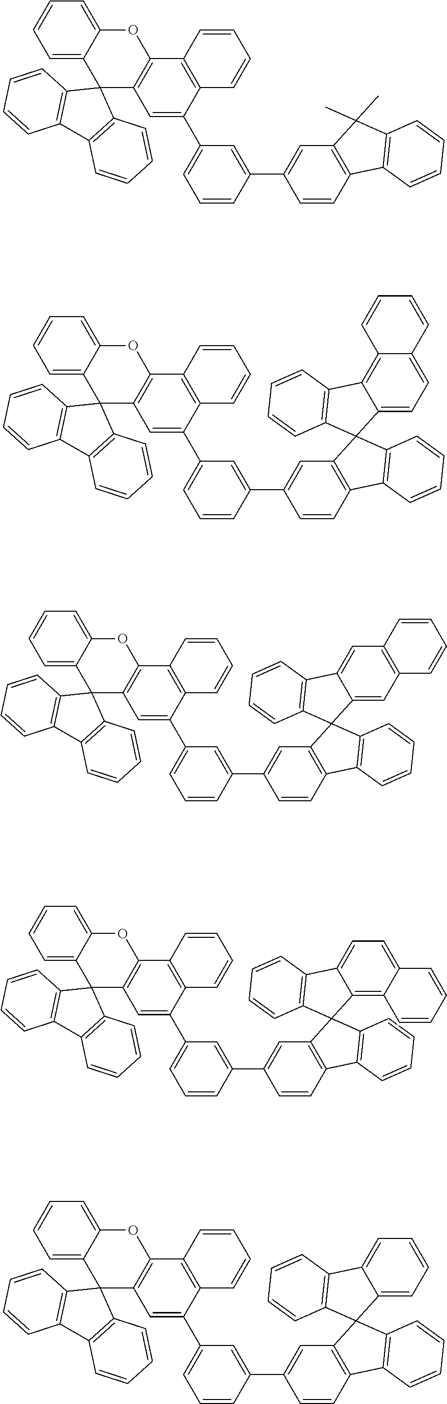

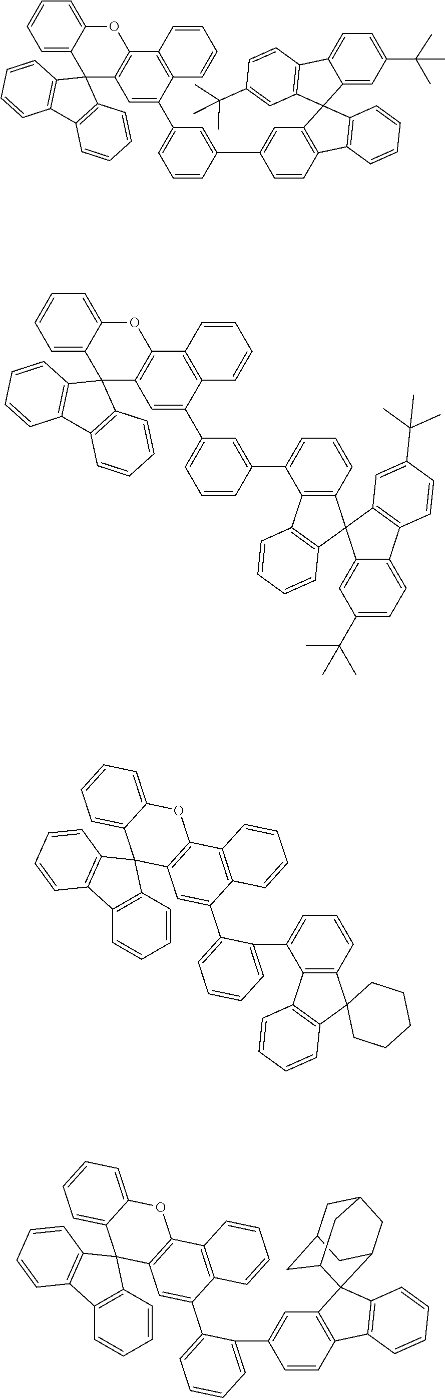 [SMILES: CC(C)(C)c1ccc2c(c1)C1(c3ccccc3-c3ccc(-c4cccc(-c5cc6c(c7ccccc57)Oc5ccccc5C65c6ccccc6-c6ccccc65)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4cc5c(c6ccccc46)Oc4ccccc4C54c5ccccc5-c5ccccc54)c3)cccc21 ; c1ccc2c(c1)Oc1c(cc(-c3ccccc3-c3cccc4c3-c3ccccc3C43CCCCC3)c3ccccc13)C21c2ccccc2-c2ccccc21 ; c1ccc2c(c1)Oc1c(cc(-c3ccccc3-c3ccc4c(c3)C3(c5ccccc5-4)C4CC5CC(C4)CC3C5)c3ccccc13)C21c2ccccc2-c2ccccc21]

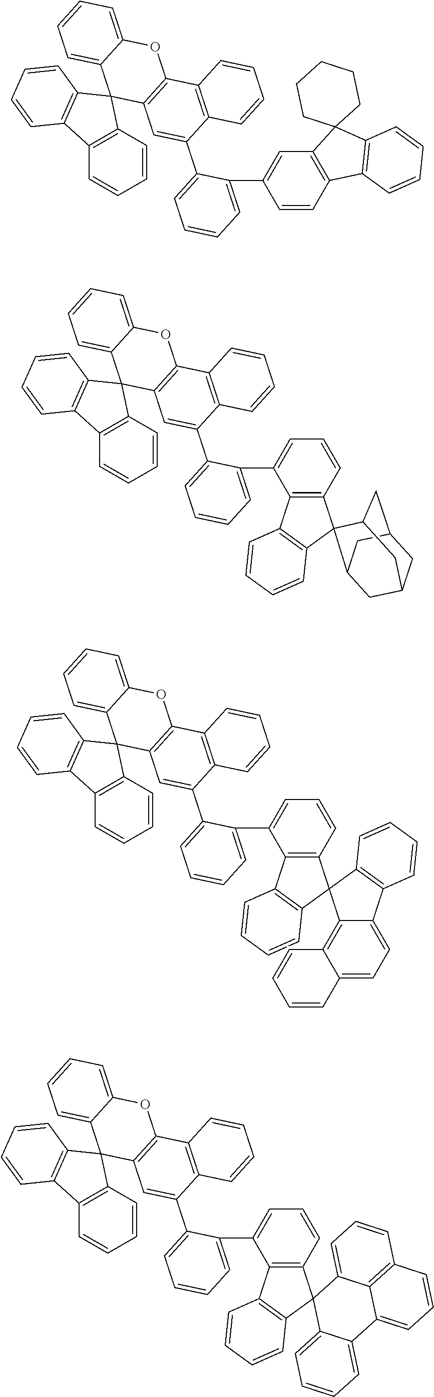 [SMILES: c1ccc2c(c1)Oc1c(cc(-c3ccccc3-c3ccc4c(c3)C3(CCCCC3)c3ccccc3-4)c3ccccc13)C21c2ccccc2-c2ccccc21 ; c1ccc2c(c1)Oc1c(cc(-c3ccccc3-c3cccc4c3-c3ccccc3C43C4CC5CC(C4)CC3C5)c3ccccc13)C21c2ccccc2-c2ccccc21 ; c1ccc2c(c1)Oc1c(cc(-c3ccccc3-c3cccc4c3-c3ccccc3C43c4ccccc4-c4ccc5ccccc5c43)c3ccccc13)C21c2ccccc2-c2ccccc21 ; c1ccc2c(c1)Oc1c(cc(-c3ccccc3-c3cccc4c3-c3ccccc3C43c4ccccc4-c4cccc5cccc3c45)c3ccccc13)C21c2ccccc2-c2ccccc21]

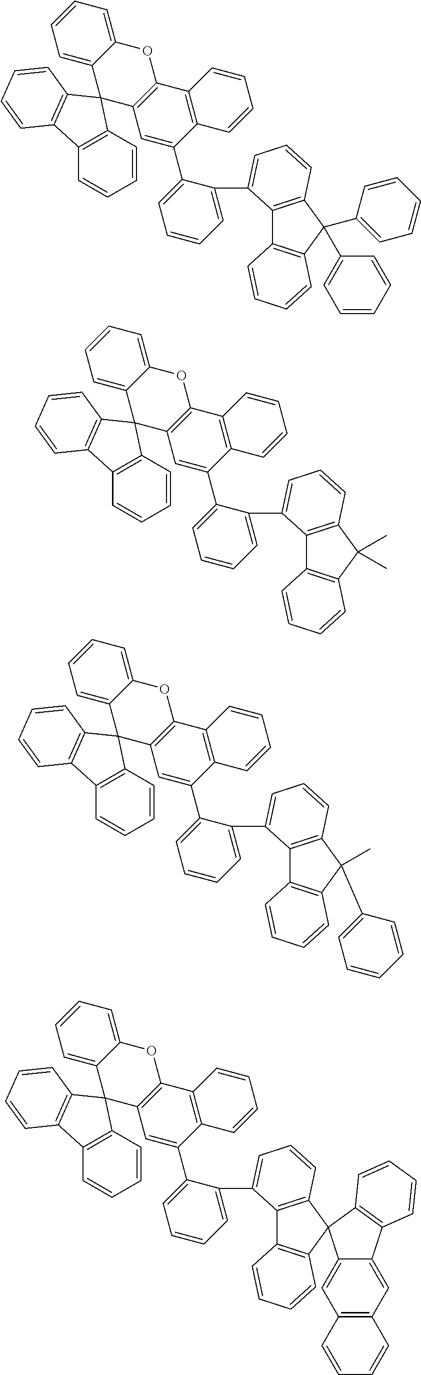 [SMILES: c1ccc(C2(c3ccccc3)c3ccccc3-c3c(-c4ccccc4-c4cc5c(c6ccccc46)Oc4ccccc4C54c5ccccc5-c5ccccc54)cccc32)cc1 ; CC1(C)c2ccccc2-c2c(-c3ccccc3-c3cc4c(c5ccccc35)Oc3ccccc3C43c4ccccc4-c4ccccc43)cccc21 ; CC1(c2ccccc2)c2ccccc2-c2c(-c3ccccc3-c3cc4c(c5ccccc35)Oc3ccccc3C43c4ccccc4-c4ccccc43)cccc21 ; c1ccc2c(c1)Oc1c(cc(-c3ccccc3-c3cccc4c3-c3ccccc3C43c4ccccc4-c4cc5ccccc5cc43)c3ccccc13)C21c2ccccc2-c2ccccc21]

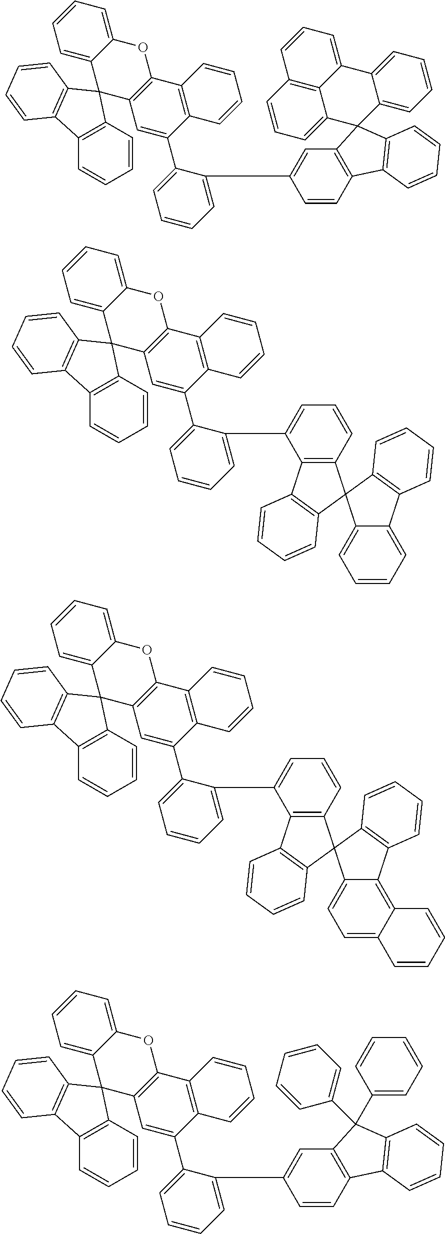 [SMILES: c1ccc2c(c1)Oc1c(cc(-c3ccccc3-c3ccc4c(c3)C3(c5ccccc5-4)c4ccccc4-c4cccc5cccc3c45)c3ccccc13)C21c2ccccc2-c2ccccc21 ; c1ccc2c(c1)Oc1c(cc(-c3ccccc3-c3cccc4c3-c3ccccc3C43c4ccccc4-c4ccccc43)c3ccccc13)C21c2ccccc2-c2ccccc21 ; c1ccc2c(c1)Oc1c(cc(-c3ccccc3-c3cccc4c3-c3ccccc3C43c4ccccc4-c4c3ccc3ccccc43)c3ccccc13)C21c2ccccc2-c2ccccc21 ; c1ccc(C2(c3ccccc3)c3ccccc3-c3ccc(-c4ccccc4-c4cc5c(c6ccccc46)Oc4ccccc4C54c5ccccc5-c5ccccc54)cc32)cc1]

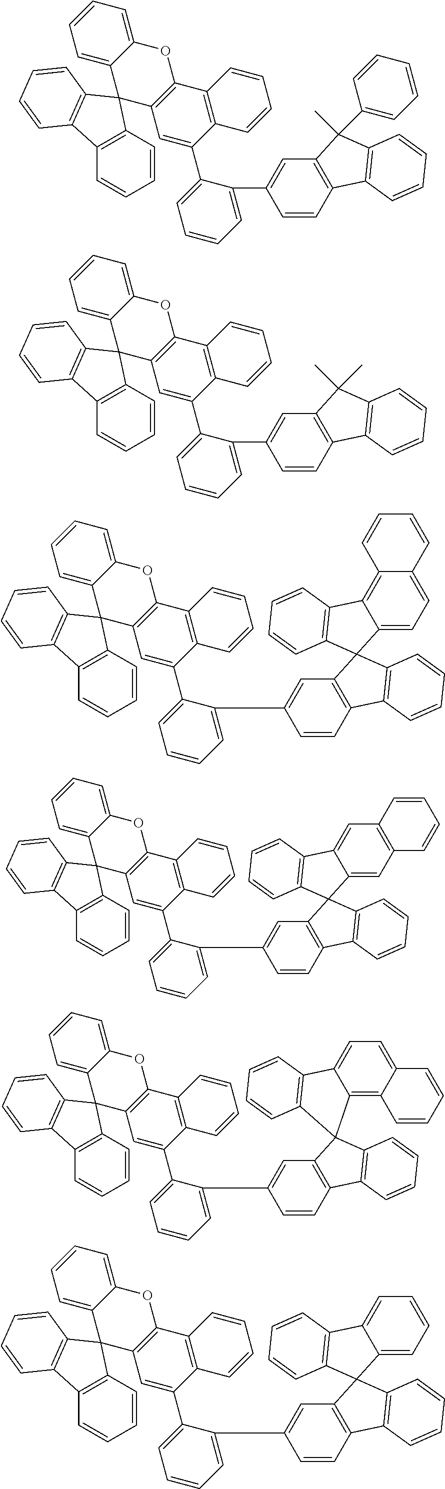

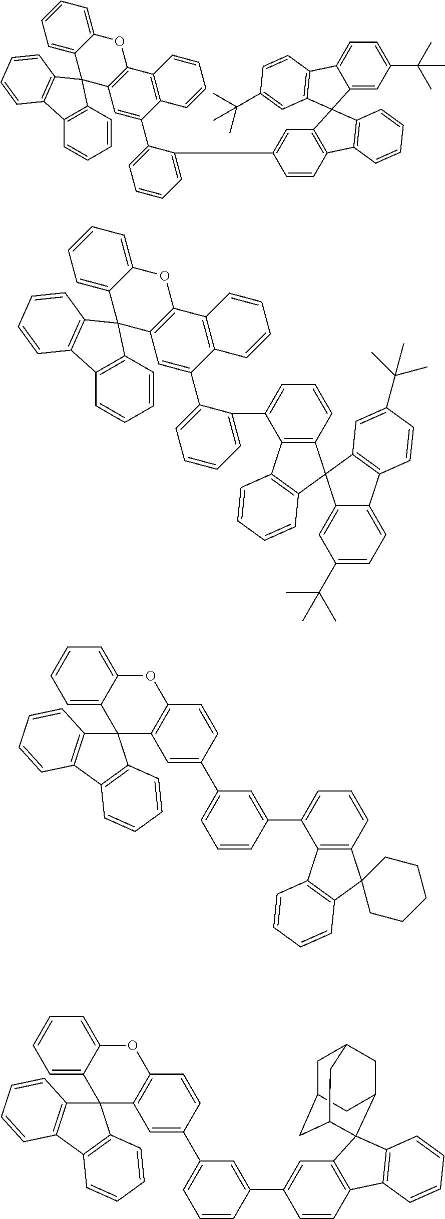

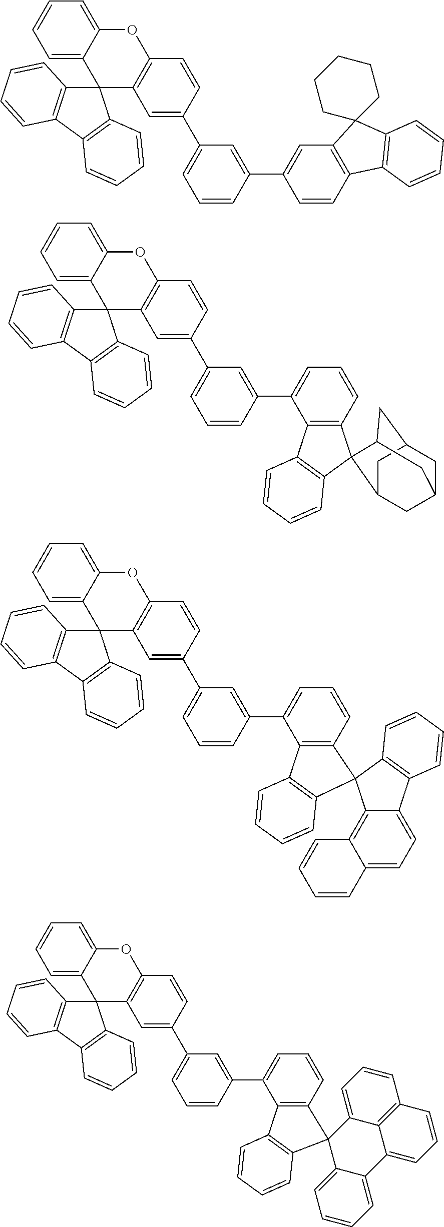

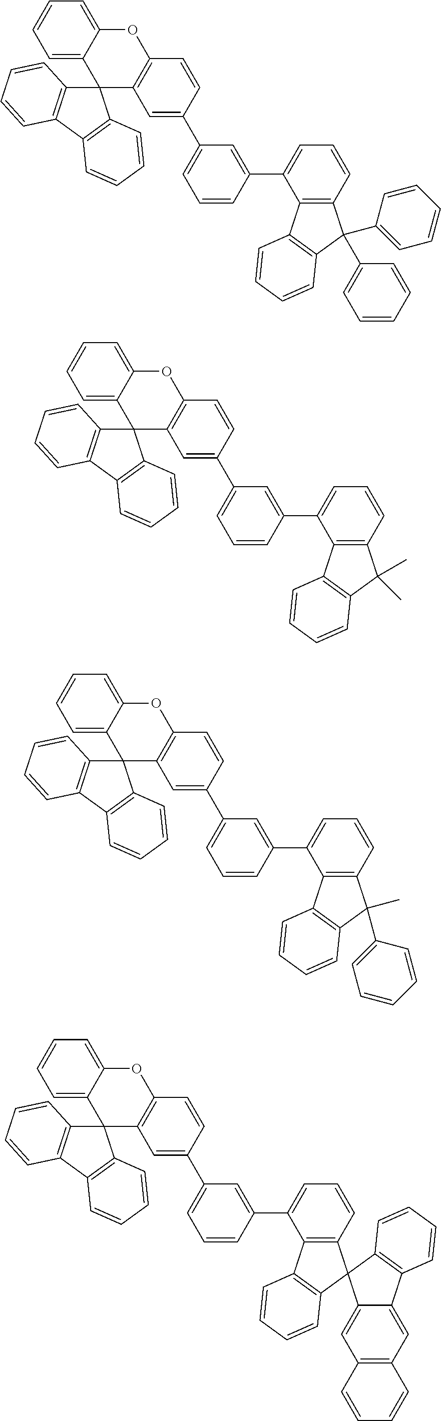

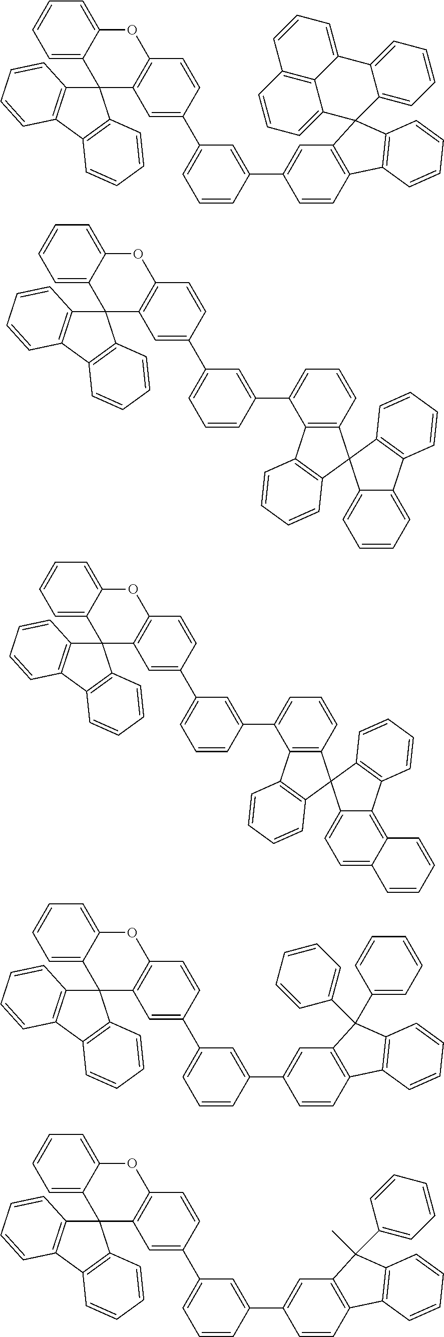

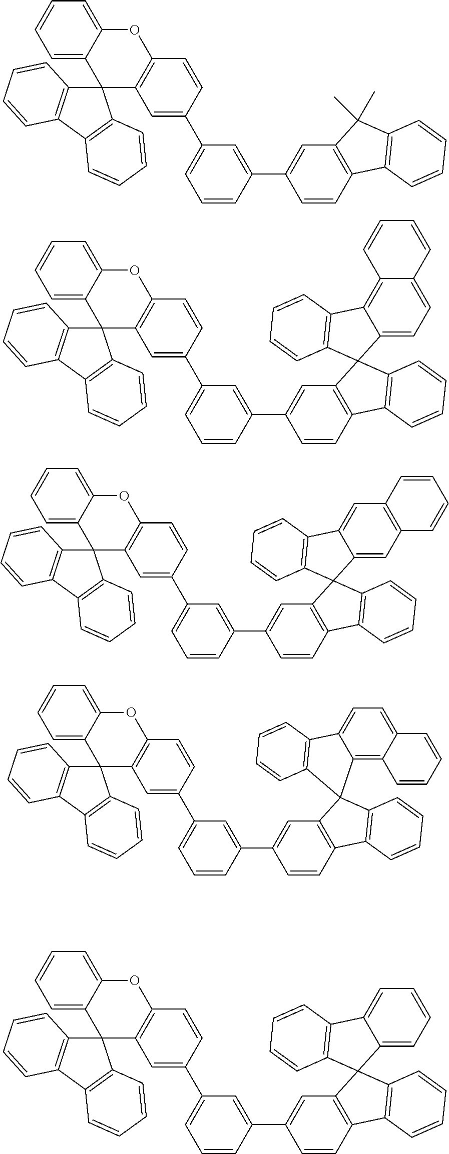

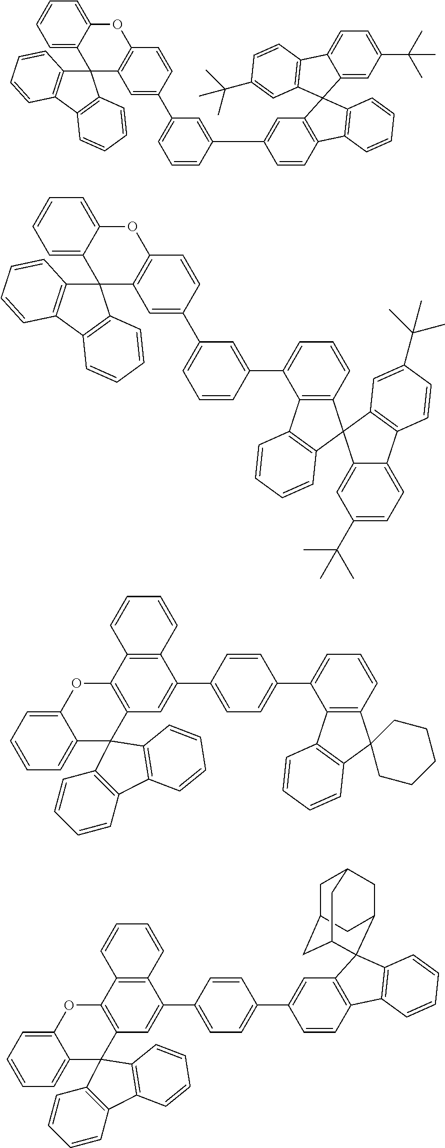 [SMILES: CC(C)(C)c1ccc2c(c1)C1(c3ccccc3-c3ccc(-c4cccc(-c5ccc6c(c5)C5(c7ccccc7O6)c6ccccc6-c6ccccc65)c4)cc31)c1cc(C(C)(C)C)ccc1-2 ; CC(C)(C)c1ccc2c(c1)C1(c3cc(C(C)(C)C)ccc3-2)c2ccccc2-c2c(-c3cccc(-c4ccc5c(c4)C4(c6ccccc6O5)c5ccccc5-c5ccccc54)c3)cccc21 ; c1ccc2c(c1)Oc1c(cc(-c3ccc(-c4cccc5c4-c4ccccc4C54CCCCC4)cc3)c3ccccc13)C21c2ccccc2-c2ccccc21 ; c1ccc2c(c1)Oc1c(cc(-c3ccc(-c4ccc5c(c4)C4(c6ccccc6-5)C5CC6CC(C5)CC4C6)cc3)c3ccccc13)C21c2ccccc2-c2ccccc21]

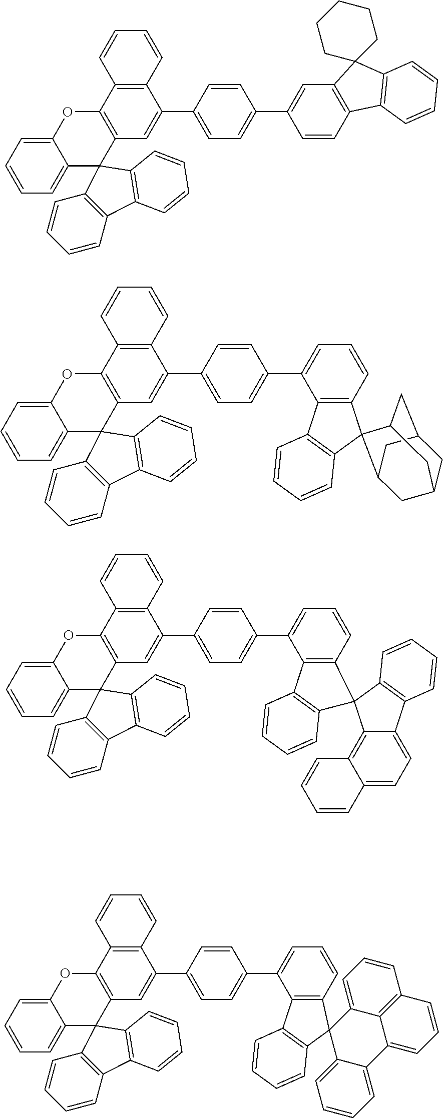

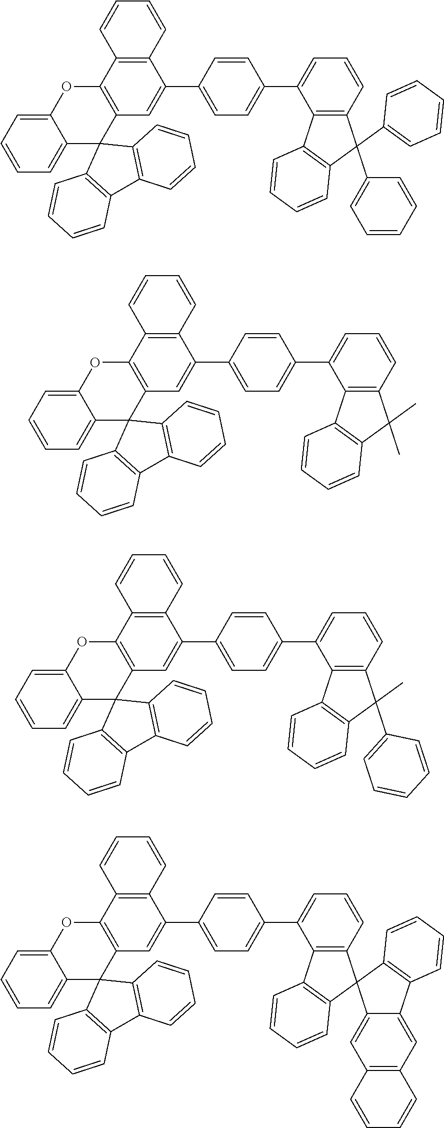 [SMILES: c1ccc(C2(c3ccccc3)c3ccccc3-c3c(-c4ccc(-c5cc6c(c7ccccc57)Oc5ccccc5C65c6ccccc6-c6ccccc65)cc4)cccc32)cc1 ; CC1(C)c2ccccc2-c2c(-c3ccc(-c4cc5c(c6ccccc46)Oc4ccccc4C54c5ccccc5-c5ccccc54)cc3)cccc21 ; CC1(c2ccccc2)c2ccccc2-c2c(-c3ccc(-c4cc5c(c6ccccc46)Oc4ccccc4C54c5ccccc5-c5ccccc54)cc3)cccc21 ; c1ccc2c(c1)Oc1c(cc(-c3ccc(-c4cccc5c4-c4ccccc4C54c5ccccc5-c5cc6ccccc6cc54)cc3)c3ccccc13)C21c2ccccc2-c2ccccc21]

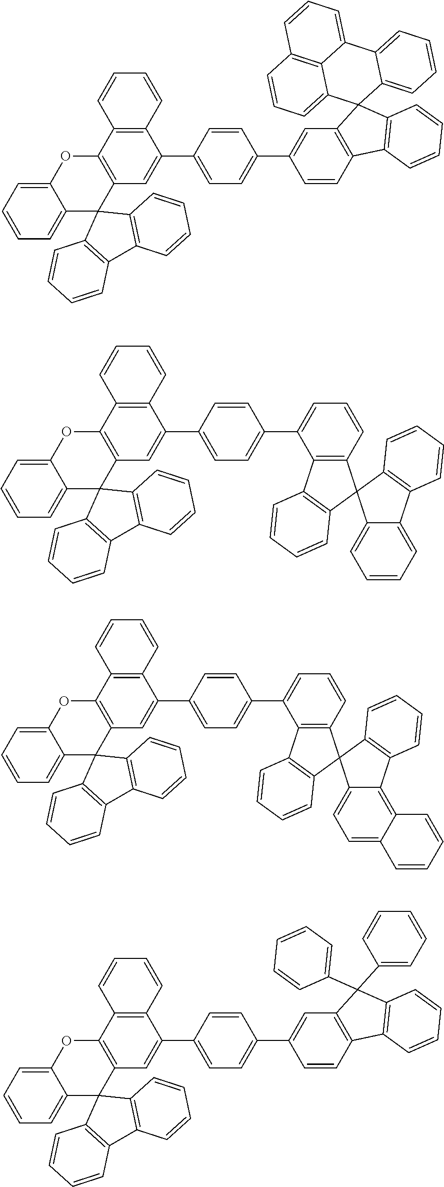 [SMILES: c1ccc2c(c1)Oc1c(cc(-c3ccc(-c4ccc5c(c4)C4(c6ccccc6-5)c5ccccc5-c5cccc6cccc4c56)cc3)c3ccccc13)C21c2ccccc2-c2ccccc21 ; c1ccc2c(c1)Oc1c(cc(-c3ccc(-c4cccc5c4-c4ccccc4C54c5ccccc5-c5ccccc54)cc3)c3ccccc13)C21c2ccccc2-c2ccccc21 ; c1ccc2c(c1)Oc1c(cc(-c3ccc(-c4cccc5c4-c4ccccc4C54c5ccccc5-c5c4ccc4ccccc54)cc3)c3ccccc13)C21c2ccccc2-c2ccccc21 ; c1ccc(C2(c3ccccc3)c3ccccc3-c3ccc(-c4ccc(-c5cc6c(c7ccccc57)Oc5ccccc5C65c6ccccc6-c6ccccc65)cc4)cc32)cc1]

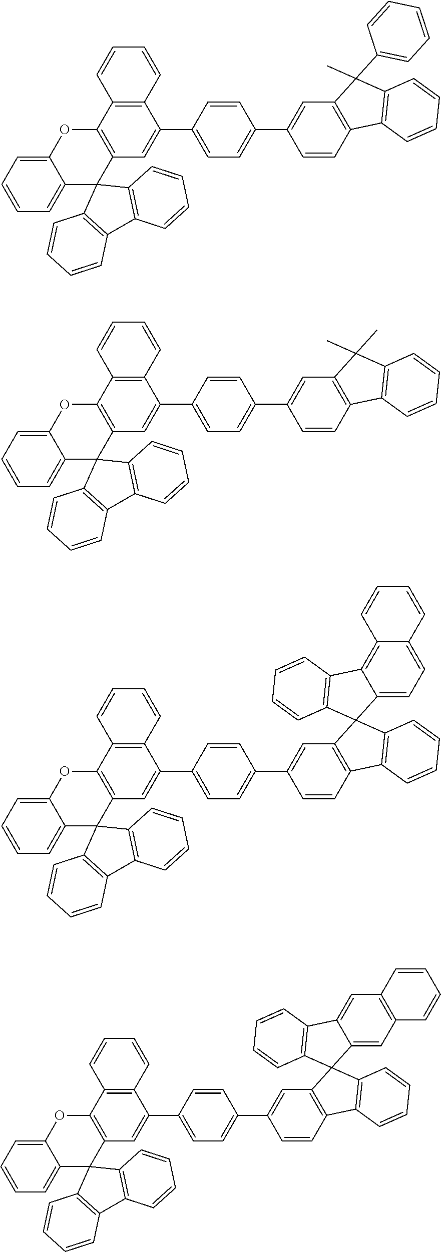 [SMILES: CC1(c2ccccc2)c2ccccc2-c2ccc(-c3ccc(-c4cc5c(c6ccccc46)Oc4ccccc4C54c5ccccc5-c5ccccc54)cc3)cc21 ; CC1(C)c2ccccc2-c2ccc(-c3ccc(-c4cc5c(c6ccccc46)Oc4ccccc4C54c5ccccc5-c5ccccc54)cc3)cc21 ; c1ccc2c(c1)Oc1c(cc(-c3ccc(-c4ccc5c(c4)C4(c6ccccc6-5)c5ccccc5-c5c4ccc4ccccc54)cc3)c3ccccc13)C21c2ccccc2-c2ccccc21 ; c1ccc2c(c1)Oc1c(cc(-c3ccc(-c4ccc5c(c4)C4(c6ccccc6-5)c5ccccc5-c5cc6ccccc6cc54)cc3)c3ccccc13)C21c2ccccc2-c2ccccc21]

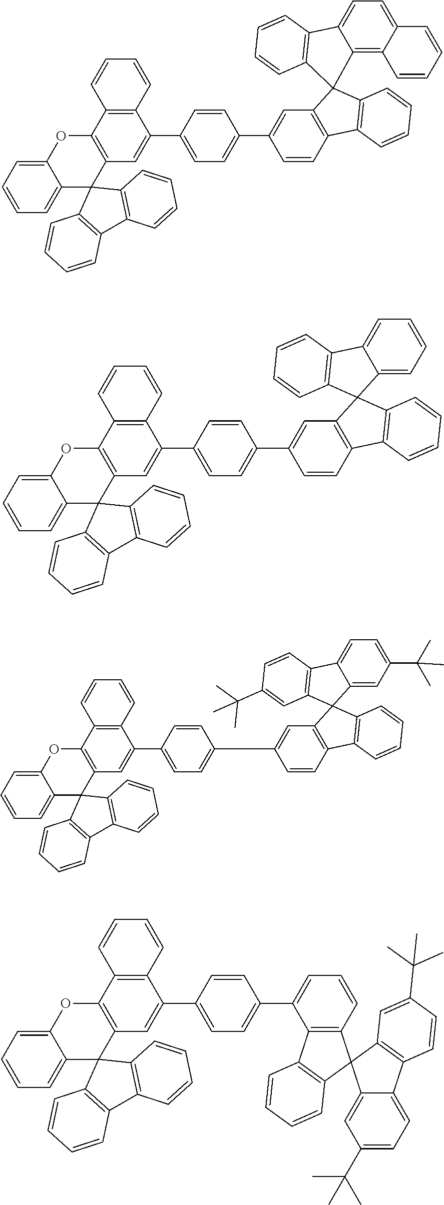 [SMILES: c1ccc2c(c1)Oc1c(cc(-c3ccc(-c4ccc5c(c4)C4(c6ccccc6-5)c5ccccc5-c5ccc6ccccc6c54)cc3)c3ccccc13)C21c2ccccc2-c2ccccc21 ; c1ccc2c(c1)Oc1c(cc(-c3ccc(-c4ccc5c(c4)C4(c6ccccc6-c6ccccc64)c4ccccc4-5)cc3)c3ccccc13)C21c2ccccc2-c2ccccc21 ; CC(C)(C)c1ccc2c(c1)C1(c3ccccc3-c3ccc(-c4ccc(-c5cc6c(c7ccccc57)Oc5ccccc5C65c6ccccc6-c6ccccc65)cc4)cc31)c1cc(C(C)(C)C)ccc1-2 ; CC(C)(C)c1ccc2c(c1)C1(c3cc(C(C)(C)C)ccc3-2)c2ccccc2-c2c(-c3ccc(-c4cc5c(c6ccccc46)Oc4ccccc4C54c5ccccc5-c5ccccc54)cc3)cccc21]

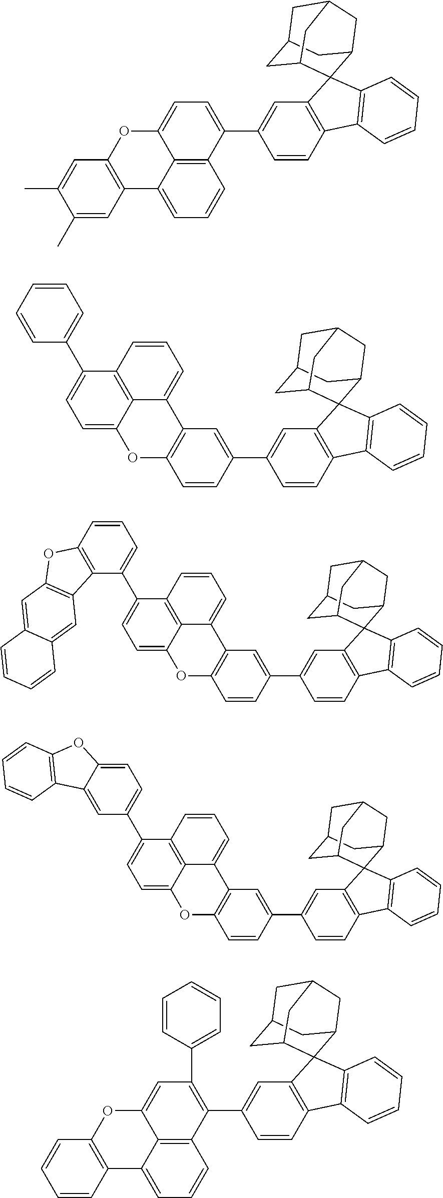

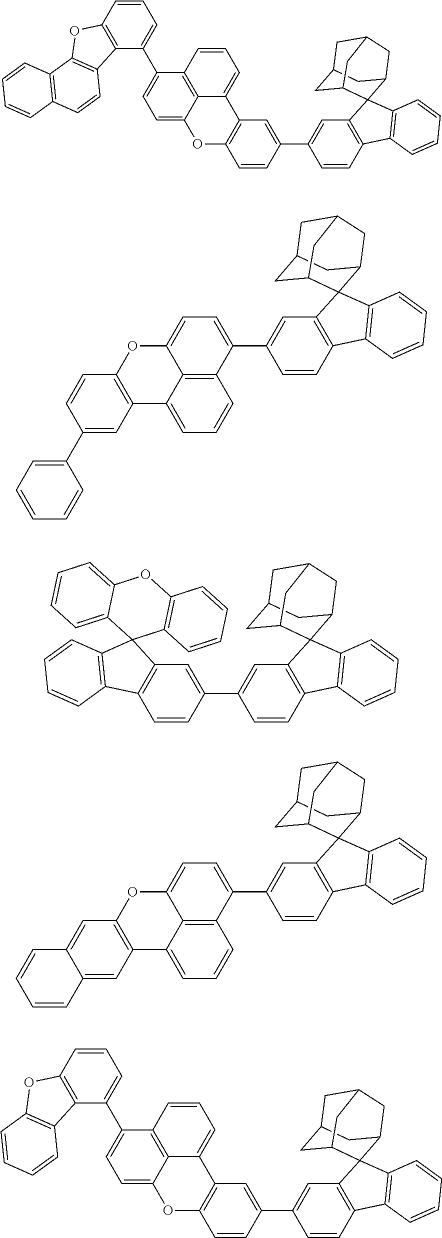

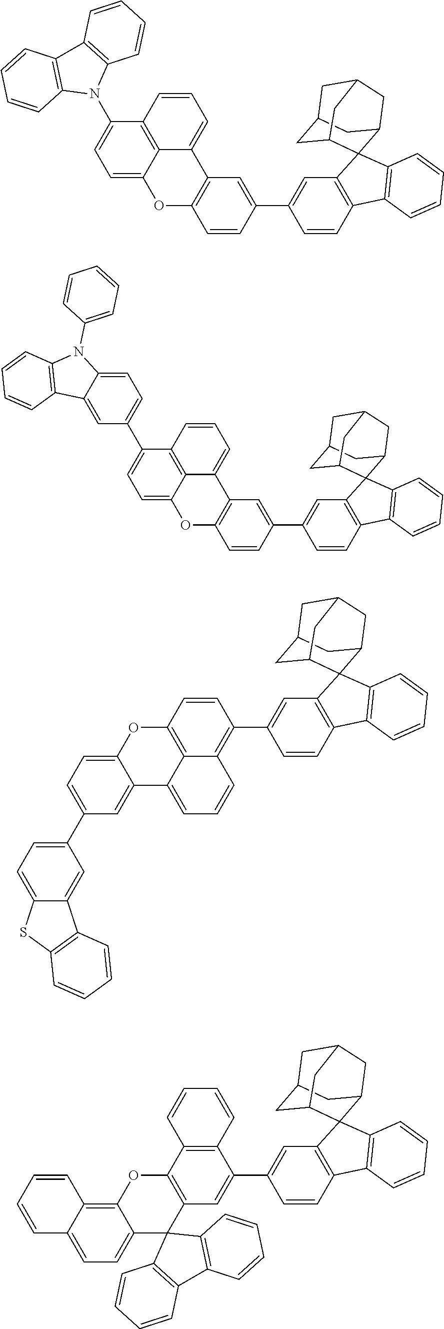 [SMILES: c1ccc2c(c1)-c1ccc(-c3ccc4c(c3)-c3cccc5c(-n6c7ccccc7c7ccccc76)ccc(c35)O4)cc1C21C2CC3CC(C2)CC1C3 ; c1ccc(-n2c3ccccc3c3cc(-c4ccc5c6c(cccc46)-c4cc(-c6ccc7c(c6)C6(c8ccccc8-7)C7CC8CC(C7)CC6C8)ccc4O5)ccc32)cc1 ; c1ccc2c(c1)-c1ccc(-c3ccc4c5c(cccc35)-c3cc(-c5ccc6sc7ccccc7c6c5)ccc3O4)cc1C21C2CC3CC(C2)CC1C3 ; c1ccc2c(c1)-c1ccccc1C21c2ccc3ccccc3c2Oc2c1cc(-c1ccc3c(c1)C1(c4ccccc4-3)C3CC4CC(C3)CC1C4)c1ccccc21]

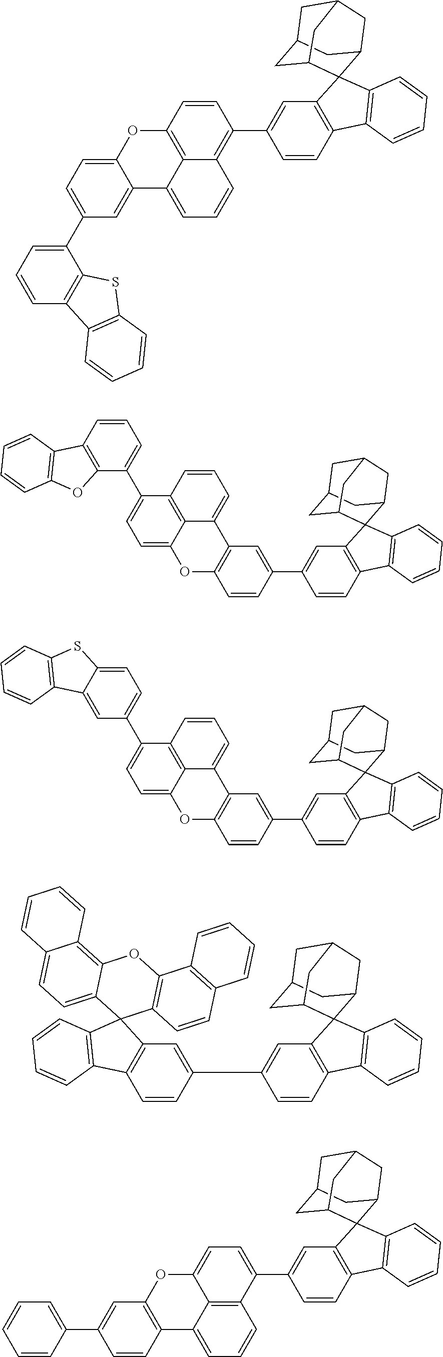

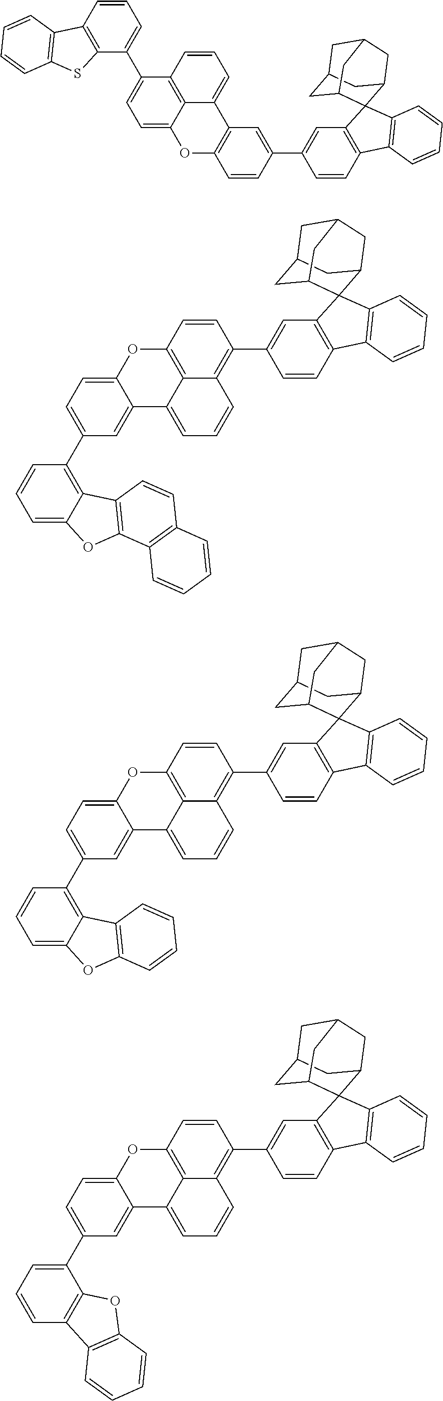

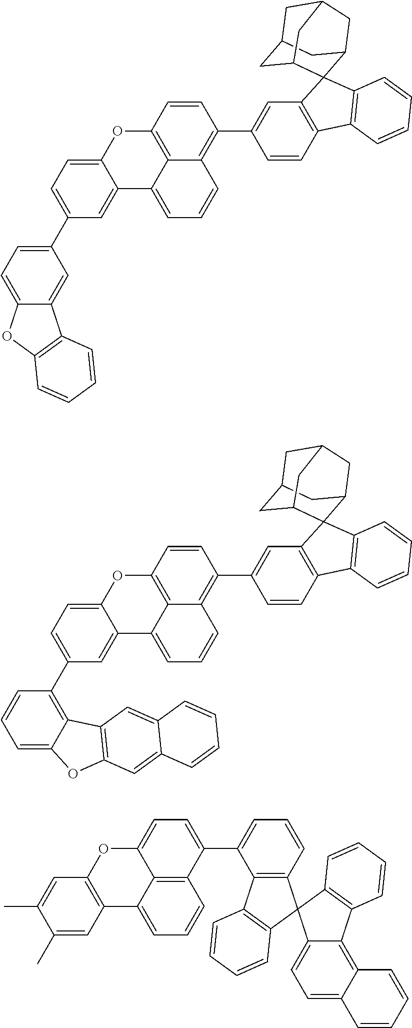 [SMILES: c1ccc2c(c1)-c1ccc(-c3ccc4c5c(cccc35)-c3cc(-c5ccc6oc7ccccc7c6c5)ccc3O4)cc1C21C2CC3CC(C2)CC1C3 ; c1ccc2c(c1)-c1ccc(-c3ccc4c5c(cccc35)-c3cc(-c5cccc6oc7cc8ccccc8cc7c56)ccc3O4)cc1C21C2CC3CC(C2)CC1C3 ; Cc1cc2c(cc1C)-c1cccc3c(-c4cccc5c4-c4ccccc4C54c5ccccc5-c5c4ccc4ccccc54)ccc(c13)O2]

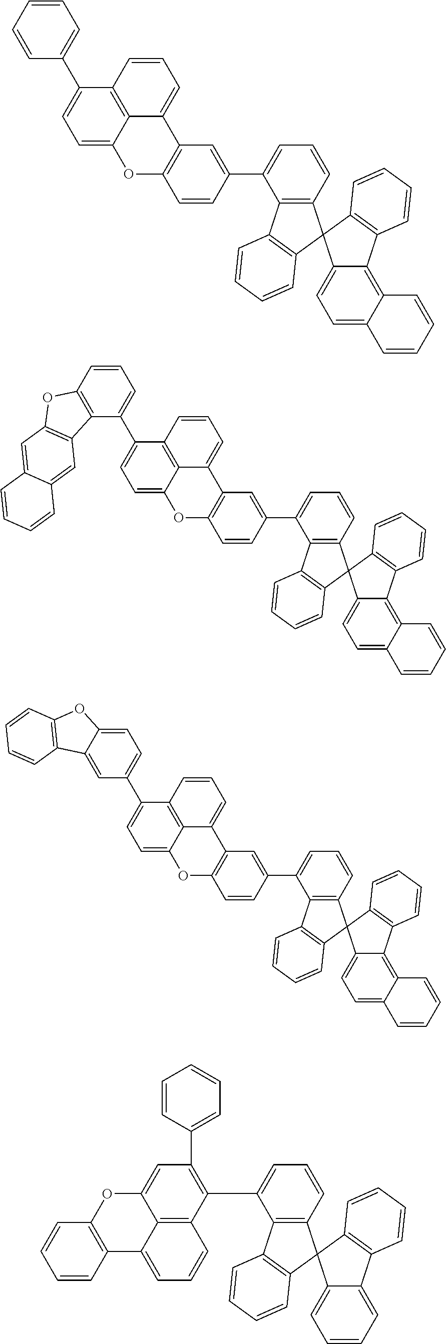 [SMILES: c1ccc(-c2ccc3c4c(cccc24)-c2cc(-c4cccc5c4-c4ccccc4C54c5ccccc5-c5c4ccc4ccccc54)ccc2O3)cc1 ; c1ccc2c(c1)-c1c(-c3ccc4c(c3)-c3cccc5c(-c6cccc7oc8cc9ccccc9cc8c67)ccc(c35)O4)cccc1C21c2ccccc2-c2c1ccc1ccccc21 ; c1ccc2c(c1)-c1c(-c3ccc4c(c3)-c3cccc5c(-c6ccc7oc8ccccc8c7c6)ccc(c35)O4)cccc1C21c2ccccc2-c2c1ccc1ccccc21 ; c1ccc(-c2cc3c4c(cccc4c2-c2cccc4c2-c2ccccc2C42c4ccccc4-c4ccccc42)-c2ccccc2O3)cc1]

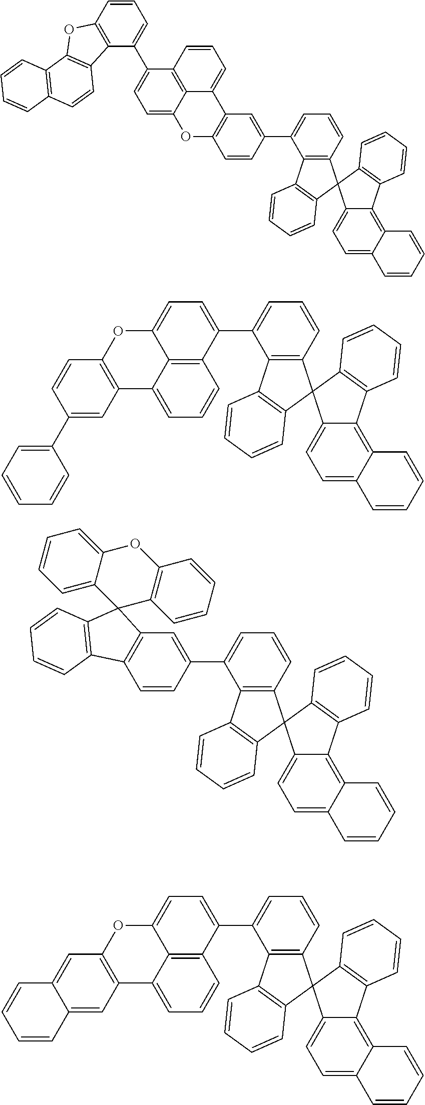

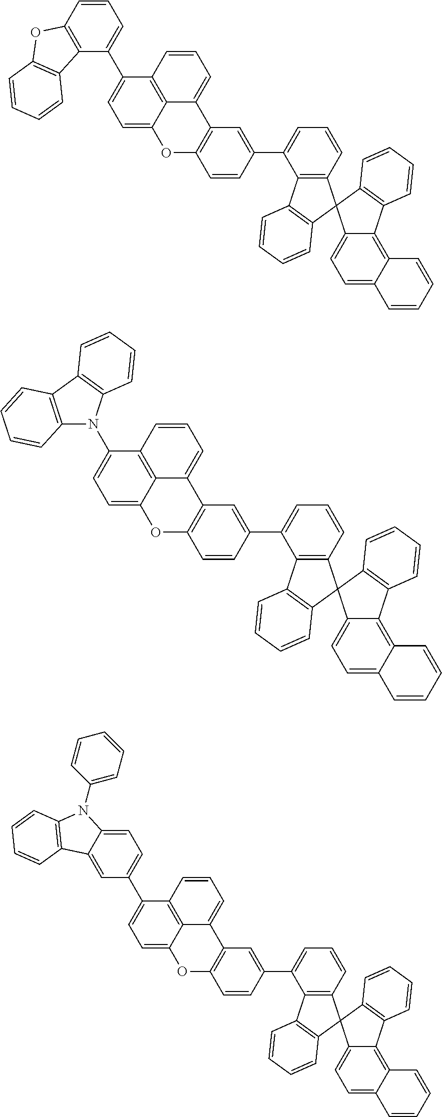 [SMILES: c1ccc2c(c1)-c1c(-c3ccc4c(c3)-c3cccc5c(-c6cccc7oc8ccccc8c67)ccc(c35)O4)cccc1C21c2ccccc2-c2c1ccc1ccccc21 ; c1ccc2c(c1)-c1c(-c3ccc4c(c3)-c3cccc5c(-n6c7ccccc7c7ccccc76)ccc(c35)O4)cccc1C21c2ccccc2-c2c1ccc1ccccc21 ; c1ccc(-n2c3ccccc3c3cc(-c4ccc5c6c(cccc46)-c4cc(-c6cccc7c6-c6ccccc6C76c7ccccc7-c7c6ccc6ccccc76)ccc4O5)ccc32)cc1]

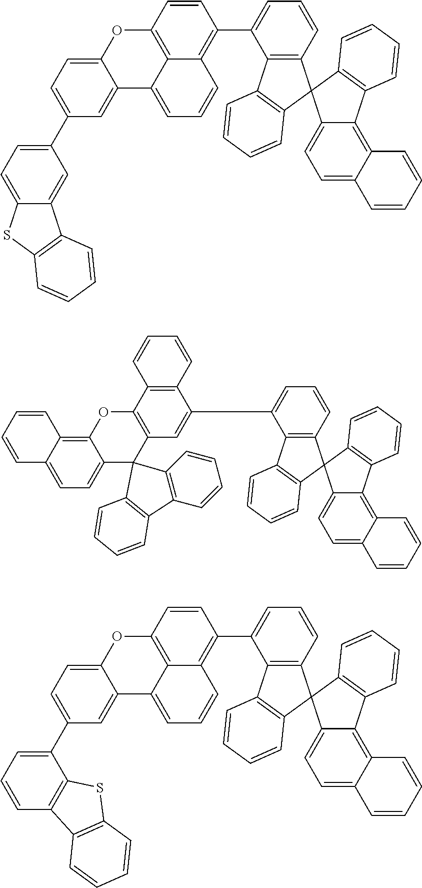 [SMILES: c1ccc2c(c1)-c1c(-c3ccc4c5c(cccc35)-c3cc(-c5ccc6sc7ccccc7c6c5)ccc3O4)cccc1C21c2ccccc2-c2c1ccc1ccccc21 ; c1ccc2c(c1)-c1c(-c3cc4c(c5ccccc35)Oc3c(ccc5ccccc35)C43c4ccccc4-c4ccccc43)cccc1C21c2ccccc2-c2c1ccc1ccccc21 ; c1ccc2c(c1)-c1c(-c3ccc4c5c(cccc35)-c3cc(-c5cccc6c5sc5ccccc56)ccc3O4)cccc1C21c2ccccc2-c2c1ccc1ccccc21]

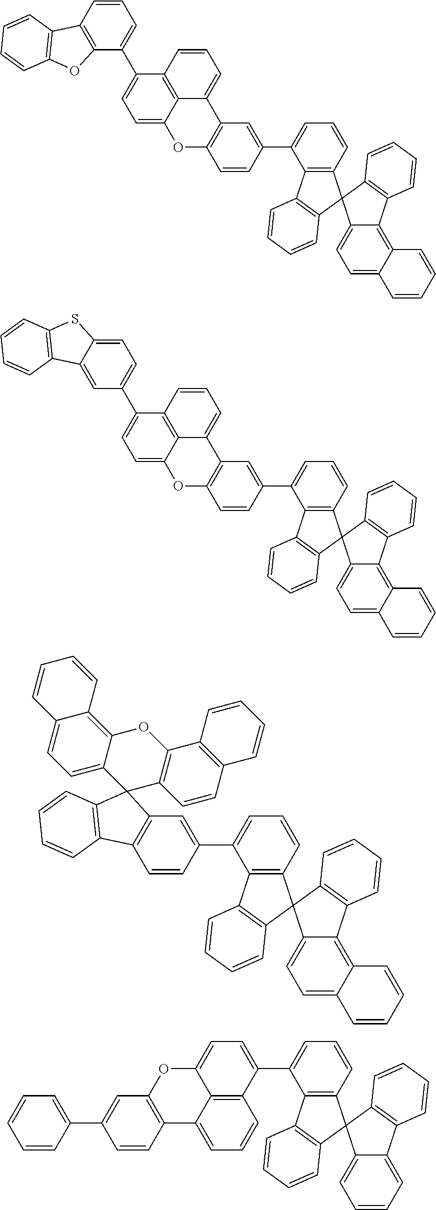

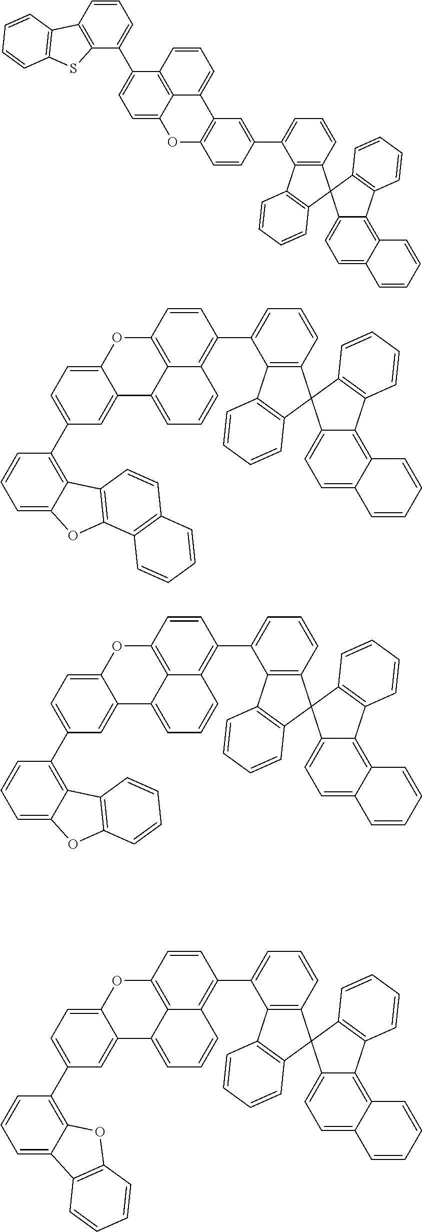

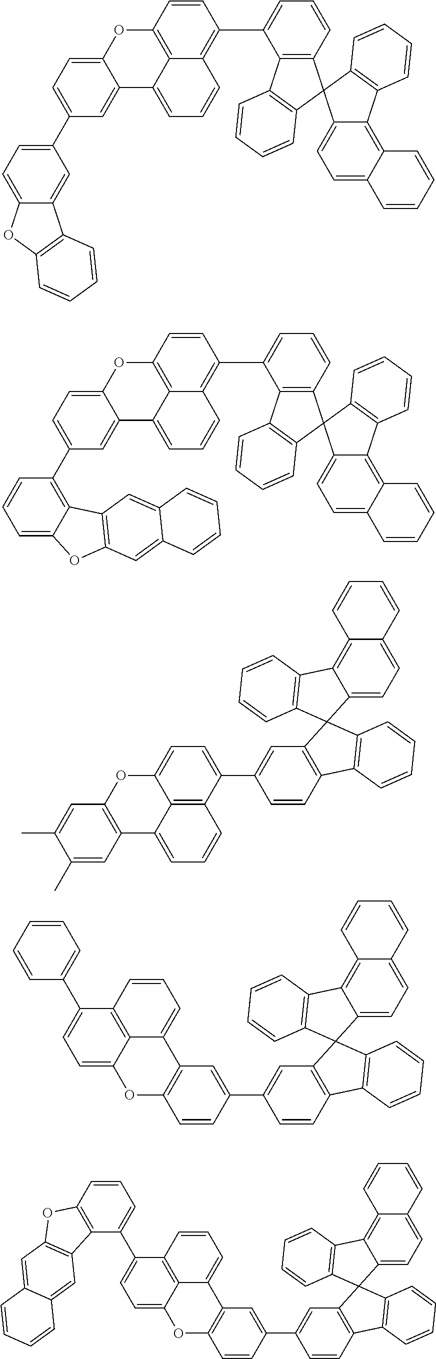

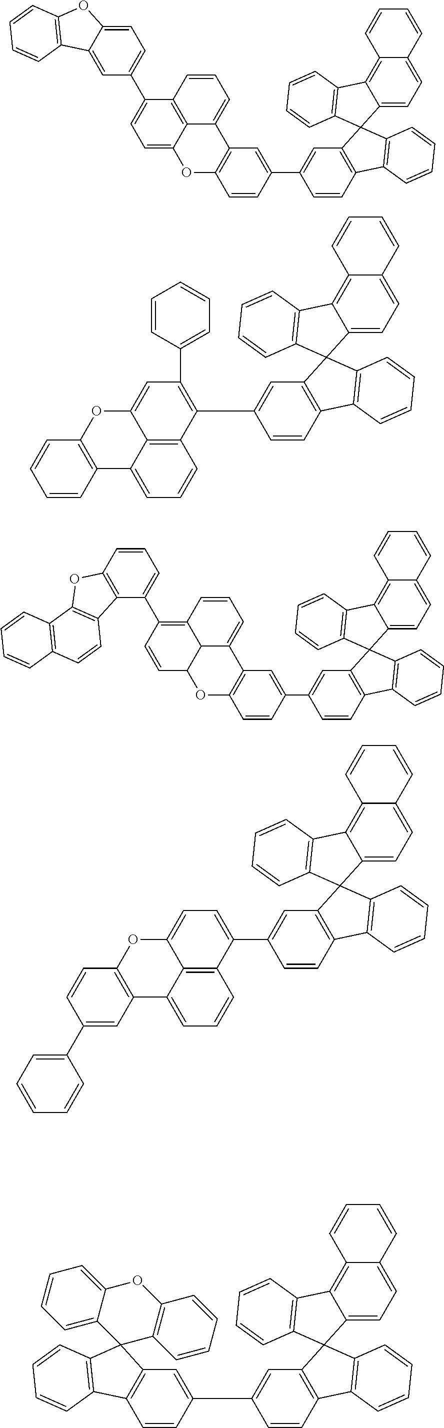

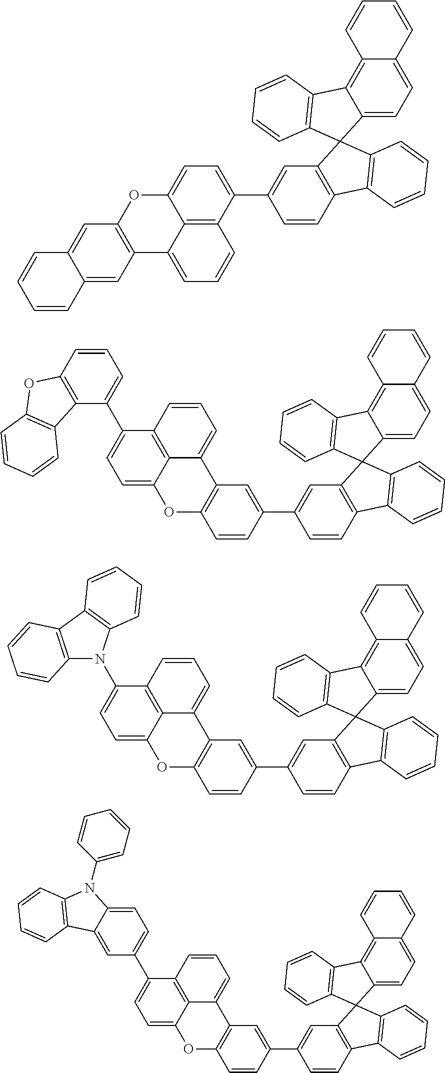

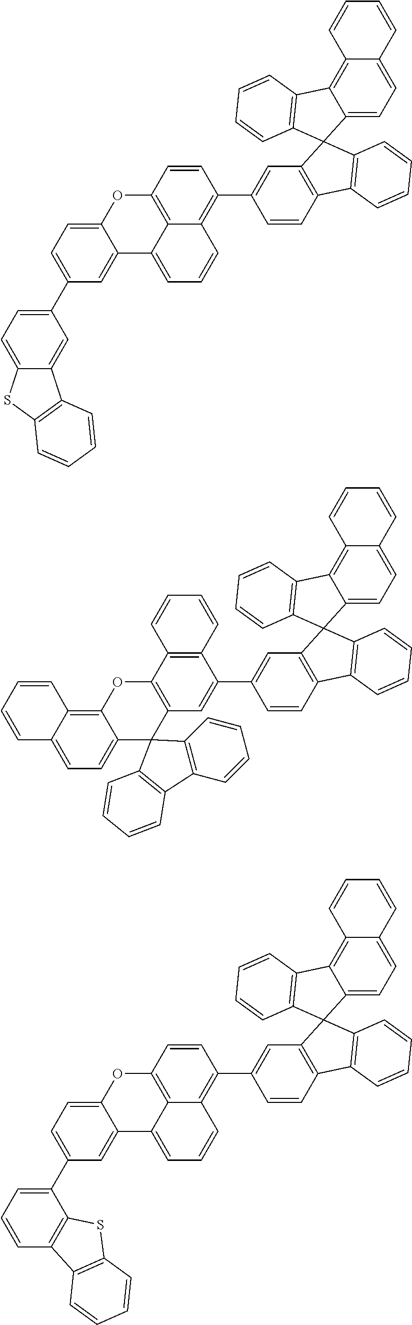 [SMILES: c1ccc2c(c1)-c1ccc(-c3ccc4c5c(cccc35)-c3cc(-c5ccc6sc7ccccc7c6c5)ccc3O4)cc1C21c2ccccc2-c2c1ccc1ccccc21 ; c1ccc2c(c1)-c1ccc(-c3cc4c(c5ccccc35)Oc3c(ccc5ccccc35)C43c4ccccc4-c4ccccc43)cc1C21c2ccccc2-c2c1ccc1ccccc21 ; c1ccc2c(c1)-c1ccc(-c3ccc4c5c(cccc35)-c3cc(-c5cccc6c5sc5ccccc56)ccc3O4)cc1C21c2ccccc2-c2c1ccc1ccccc21]

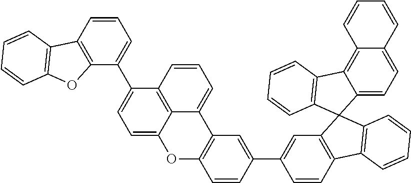 [SMILES: c1ccc2c(c1)-c1ccc(-c3ccc4c(c3)-c3cccc5c(-c6cccc7c6oc6ccccc67)ccc(c35)O4)cc1C21c2ccccc2-c2c1ccc1ccccc21]

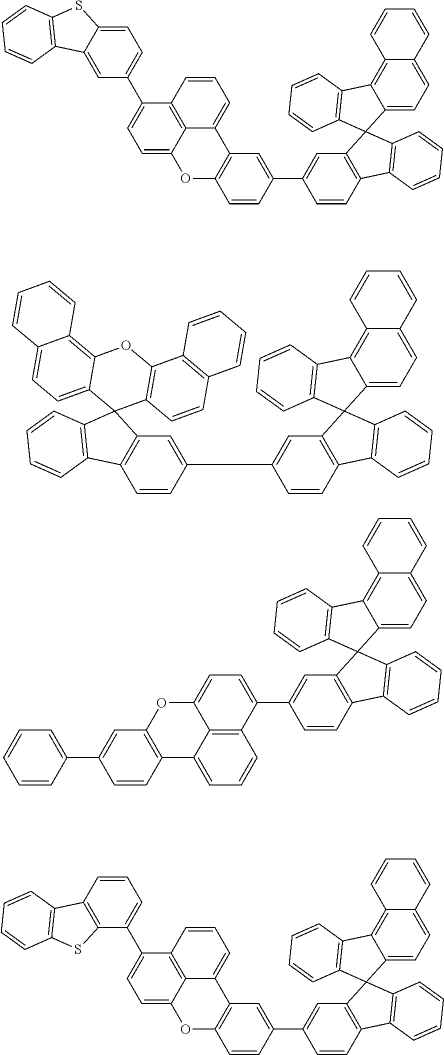 [SMILES: c1ccc2c(c1)-c1ccc(-c3ccc4c(c3)-c3cccc5c(-c6ccc7sc8ccccc8c7c6)ccc(c35)O4)cc1C21c2ccccc2-c2c1ccc1ccccc21 ; c1ccc2c(c1)-c1ccc(-c3ccc4c(c3)C3(c5ccccc5-4)c4ccccc4-c4c3ccc3ccccc43)cc1C21c2ccc3ccccc3c2Oc2c1ccc1ccccc21 ; c1ccc(-c2ccc3c(c2)Oc2ccc(-c4ccc5c(c4)C4(c6ccccc6-5)c5ccccc5-c5c4ccc4ccccc54)c4cccc-3c24)cc1 ; c1ccc2c(c1)-c1ccc(-c3ccc4c(c3)-c3cccc5c(-c6cccc7c6sc6ccccc67)ccc(c35)O4)cc1C21c2ccccc2-c2c1ccc1ccccc21]

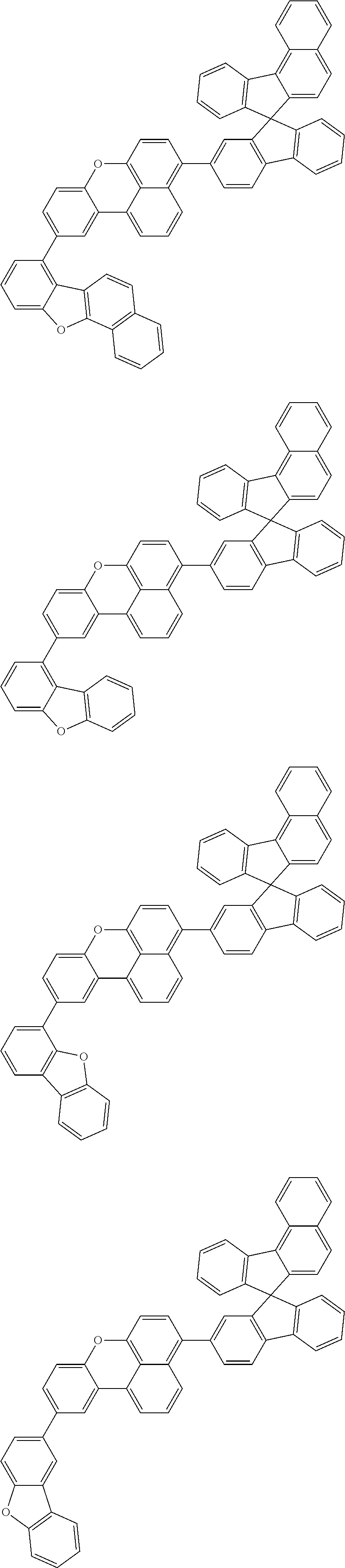 [SMILES: c1ccc2c(c1)-c1ccc(-c3ccc4c5c(cccc35)-c3cc(-c5cccc6oc7c8ccccc8ccc7c56)ccc3O4)cc1C21c2ccccc2-c2c1ccc1ccccc21 ; c1ccc2c(c1)-c1ccc(-c3ccc4c5c(cccc35)-c3cc(-c5cccc6oc7ccccc7c56)ccc3O4)cc1C21c2ccccc2-c2c1ccc1ccccc21 ; c1ccc2c(c1)-c1ccc(-c3ccc4c5c(cccc35)-c3cc(-c5cccc6c5oc5ccccc56)ccc3O4)cc1C21c2ccccc2-c2c1ccc1ccccc21 ; c1ccc2c(c1)-c1ccc(-c3ccc4c5c(cccc35)-c3cc(-c5ccc6oc7ccccc7c6c5)ccc3O4)cc1C21c2ccccc2-c2c1ccc1ccccc21]

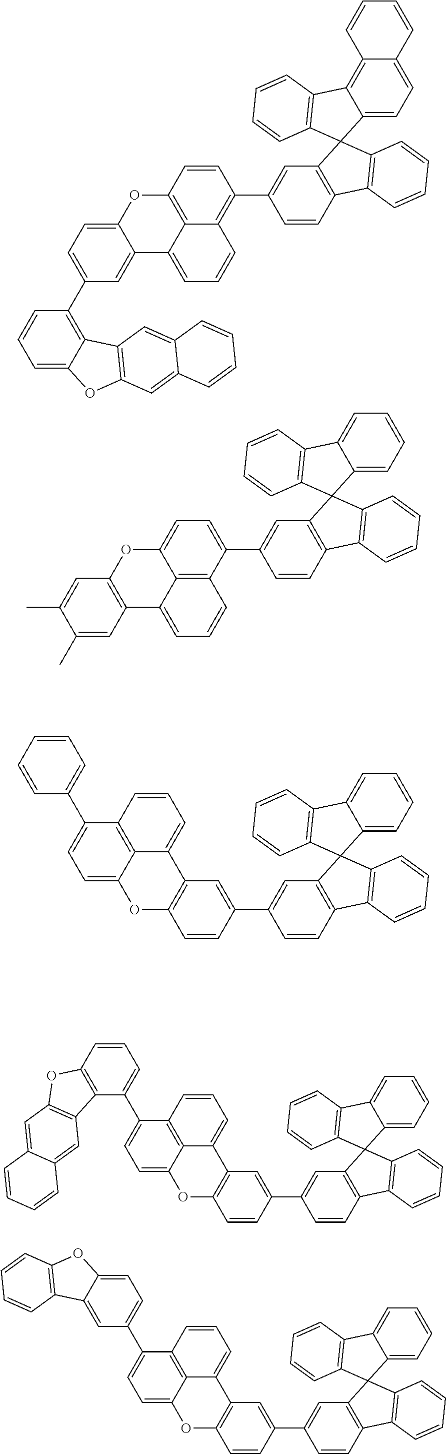

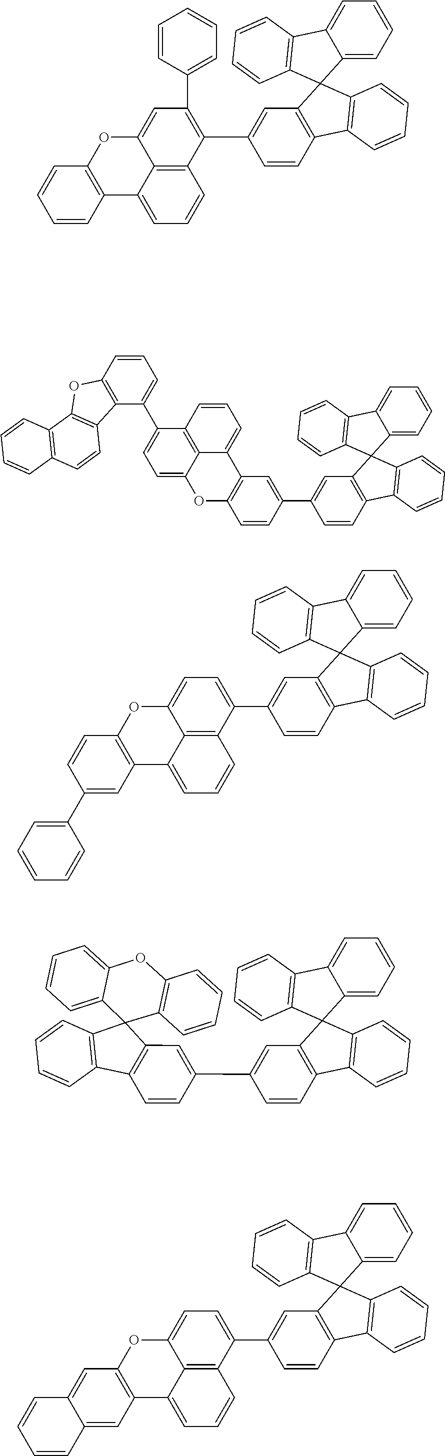

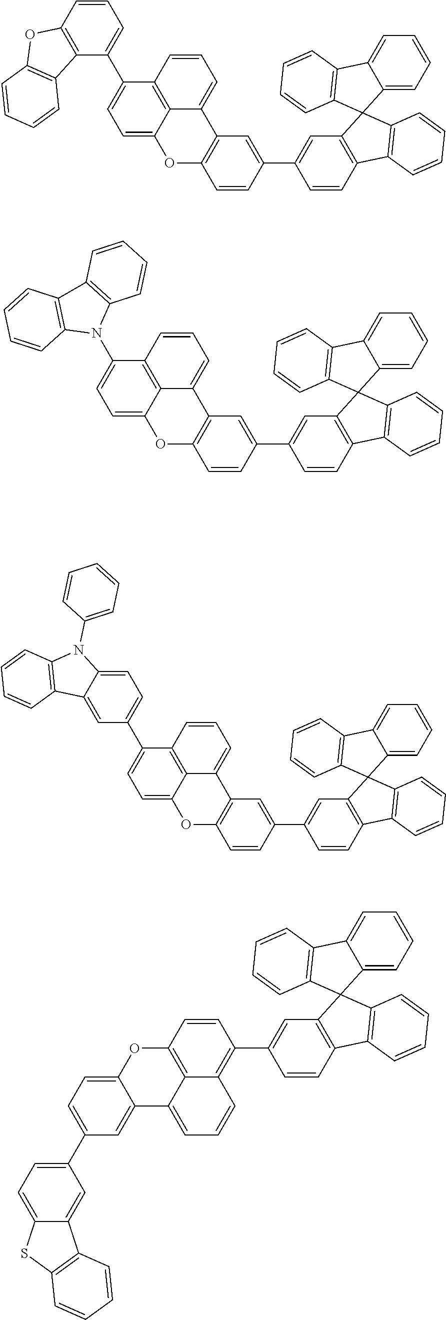 [SMILES: c1ccc2c(c1)-c1ccccc1C21c2ccccc2-c2ccc(-c3ccc4c(c3)-c3cccc5c(-c6cccc7oc8ccccc8c67)ccc(c35)O4)cc21 ; c1ccc2c(c1)-c1ccccc1C21c2ccccc2-c2ccc(-c3ccc4c(c3)-c3cccc5c(-n6c7ccccc7c7ccccc76)ccc(c35)O4)cc21 ; c1ccc(-n2c3ccccc3c3cc(-c4ccc5c6c(cccc46)-c4cc(-c6ccc7c(c6)C6(c8ccccc8-c8ccccc86)c6ccccc6-7)ccc4O5)ccc32)cc1 ; c1ccc2c(c1)-c1ccccc1C21c2ccccc2-c2ccc(-c3ccc4c5c(cccc35)-c3cc(-c5ccc6sc7ccccc7c6c5)ccc3O4)cc21]

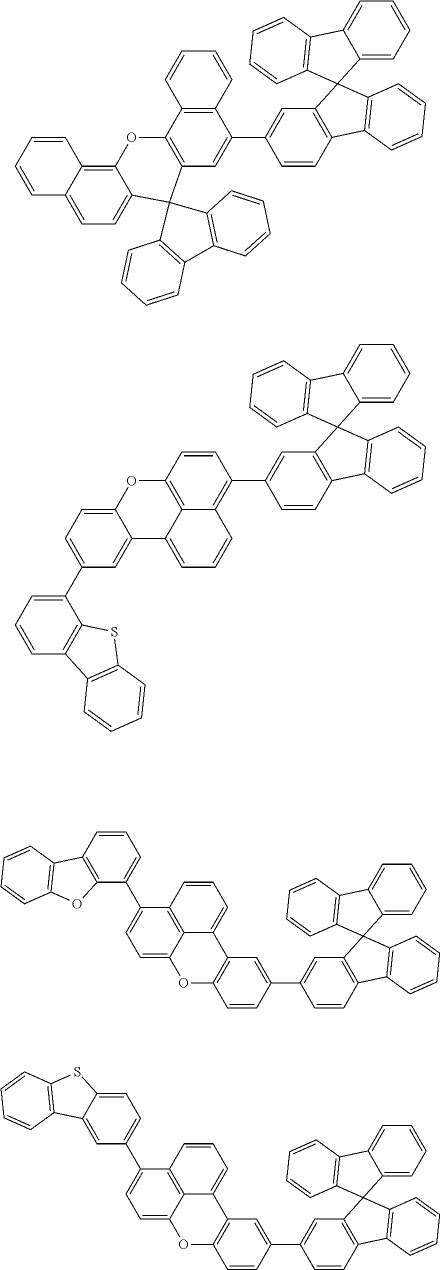 [SMILES: c1ccc2c(c1)-c1ccccc1C21c2ccccc2-c2ccc(-c3cc4c(c5ccccc35)Oc3c(ccc5ccccc35)C43c4ccccc4-c4ccccc43)cc21 ; c1ccc2c(c1)-c1ccccc1C21c2ccccc2-c2ccc(-c3ccc4c5c(cccc35)-c3cc(-c5cccc6c5sc5ccccc56)ccc3O4)cc21 ; c1ccc2c(c1)-c1ccccc1C21c2ccccc2-c2ccc(-c3ccc4c(c3)-c3cccc5c(-c6cccc7c6oc6ccccc67)ccc(c35)O4)cc21 ; c1ccc2c(c1)-c1ccccc1C21c2ccccc2-c2ccc(-c3ccc4c(c3)-c3cccc5c(-c6ccc7sc8ccccc8c7c6)ccc(c35)O4)cc21]

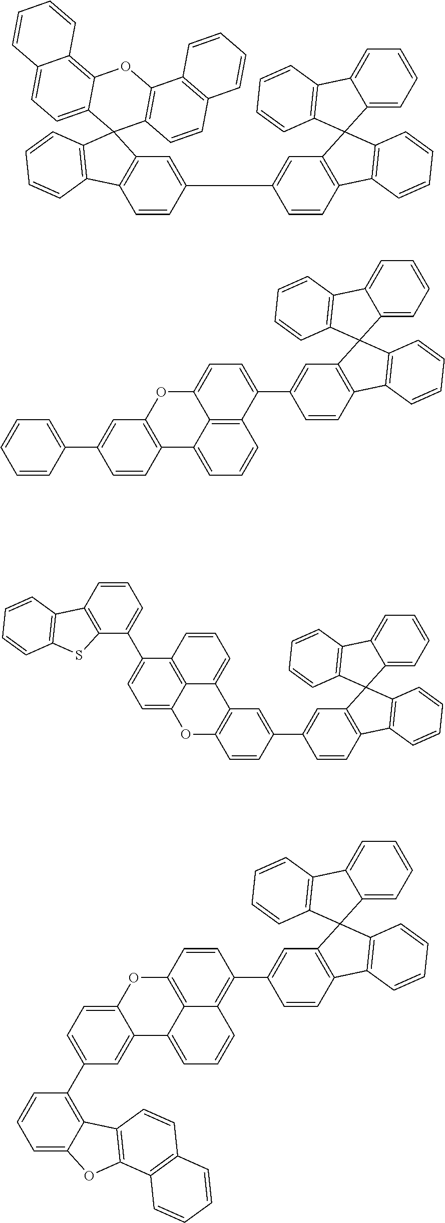 [SMILES: c1ccc2c(c1)-c1ccccc1C21c2ccccc2-c2ccc(-c3ccc4c(c3)C3(c5ccccc5-4)c4ccc5ccccc5c4Oc4c3ccc3ccccc43)cc21 ; c1ccc(-c2ccc3c(c2)Oc2ccc(-c4ccc5c(c4)C4(c6ccccc6-c6ccccc64)c4ccccc4-5)c4cccc-3c24)cc1 ; c1ccc2c(c1)-c1ccccc1C21c2ccccc2-c2ccc(-c3ccc4c(c3)-c3cccc5c(-c6cccc7c6sc6ccccc67)ccc(c35)O4)cc21 ; c1ccc2c(c1)-c1ccccc1C21c2ccccc2-c2ccc(-c3ccc4c5c(cccc35)-c3cc(-c5cccc6oc7c8ccccc8ccc7c56)ccc3O4)cc21]

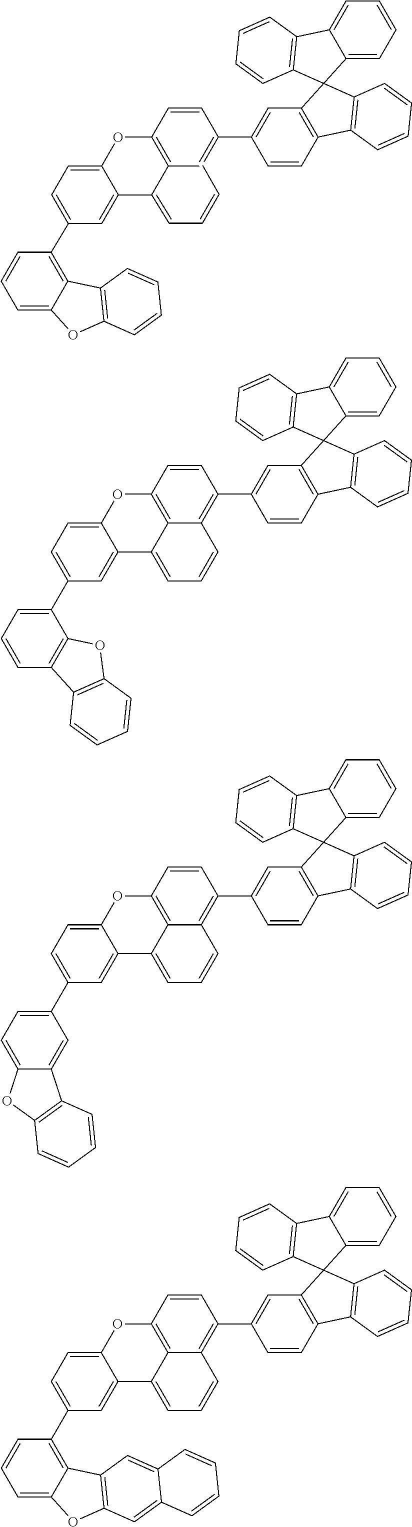 [SMILES: C/C=C\C=C1/c2cc(-c3ccc4c(c3)C3(c5ccccc5-c5ccccc53)c3ccccc3-4)ccc2Oc2ccc(-c3cccc4oc5ccccc5c34)cc21 ; c1ccc2c(c1)-c1ccccc1C21c2ccccc2-c2ccc(-c3ccc4c5c(cccc35)-c3cc(-c5cccc6c5oc5ccccc56)ccc3O4)cc21 ; c1ccc2c(c1)-c1ccccc1C21c2ccccc2-c2ccc(-c3ccc4c5c(cccc35)-c3cc(-c5ccc6oc7ccccc7c6c5)ccc3O4)cc21 ; c1ccc2c(c1)-c1ccccc1C21c2ccccc2-c2ccc(-c3ccc4c5c(cccc35)-c3cc(-c5cccc6oc7cc8ccccc8cc7c56)ccc3O4)cc21]

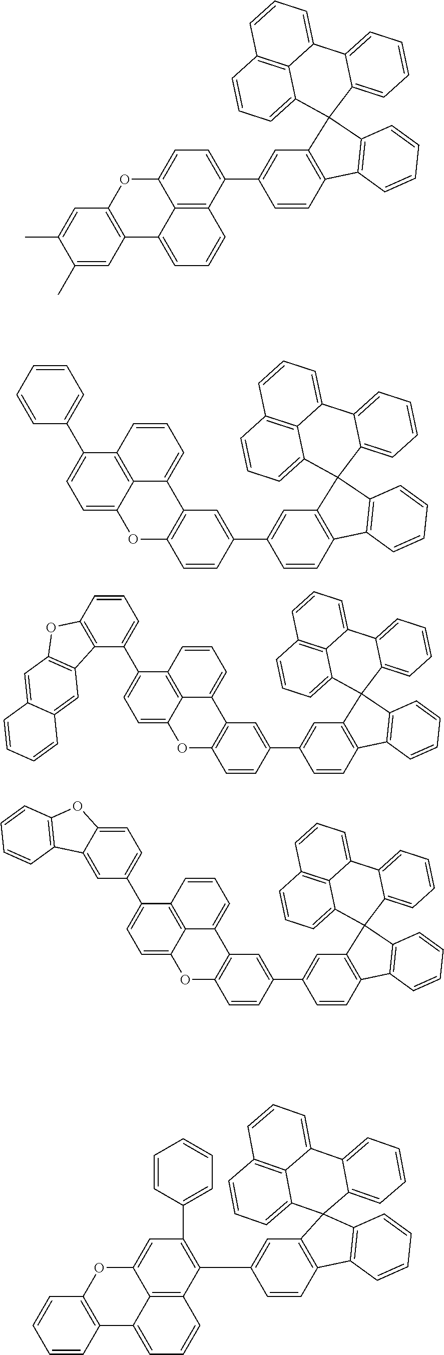

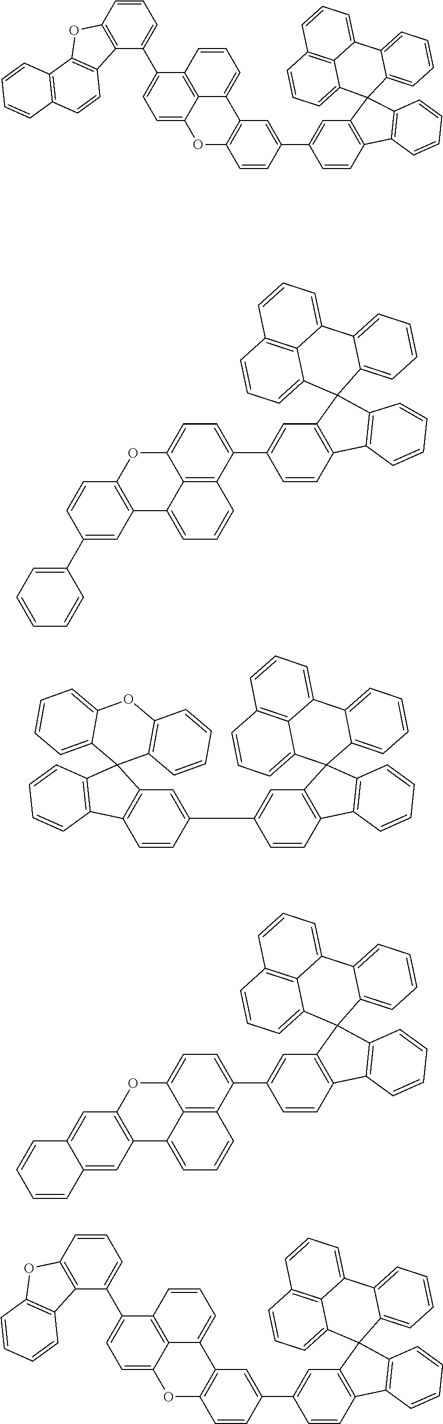

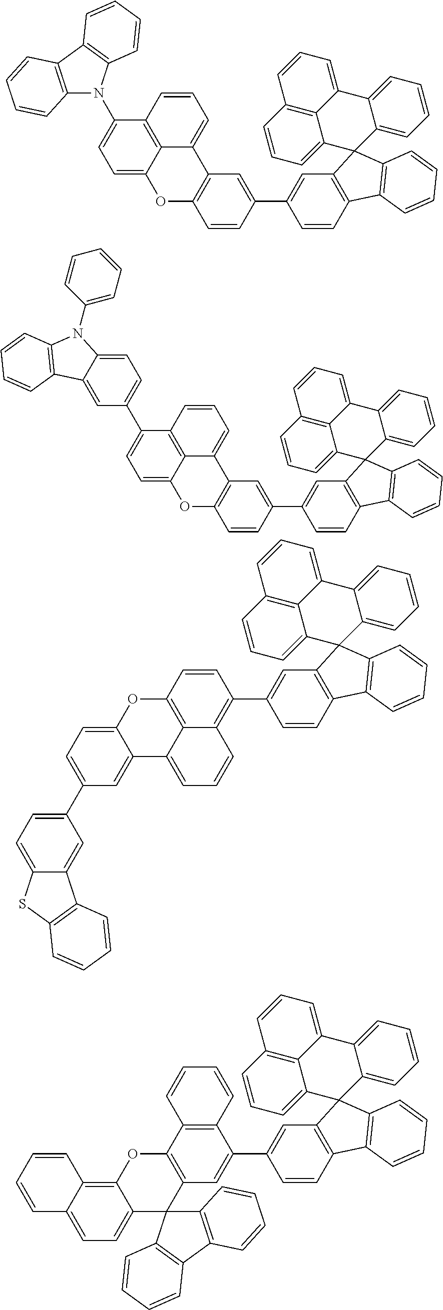

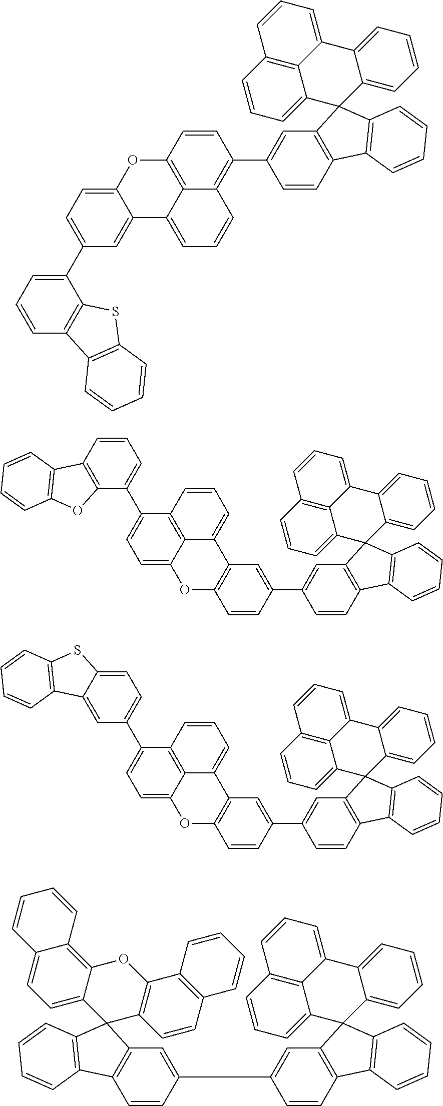

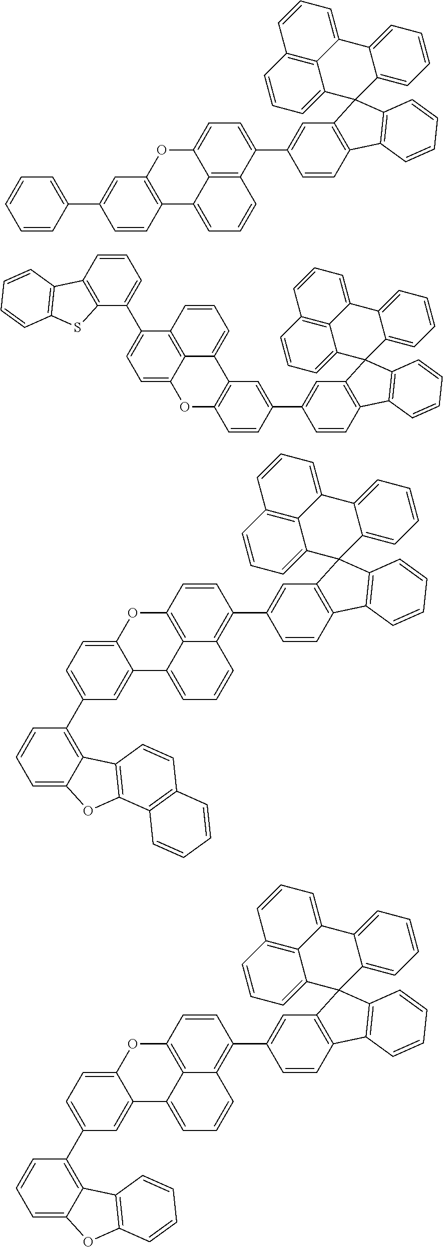

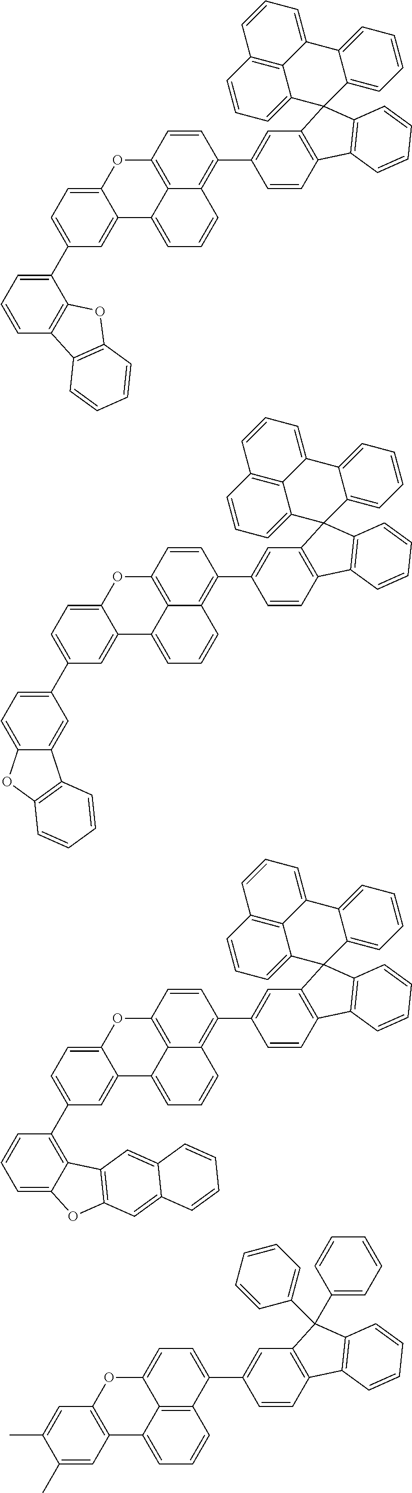 [SMILES: c1ccc2c(c1)-c1ccc(-c3ccc4c5c(cccc35)-c3cc(-c5cccc6c5oc5ccccc56)ccc3O4)cc1C21c2ccccc2-c2cccc3cccc1c23 ; c1ccc2c(c1)-c1ccc(-c3ccc4c5c(cccc35)-c3cc(-c5ccc6oc7ccccc7c6c5)ccc3O4)cc1C21c2ccccc2-c2cccc3cccc1c23 ; c1ccc2c(c1)-c1ccc(-c3ccc4c5c(cccc35)-c3cc(-c5cccc6oc7cc8ccccc8cc7c56)ccc3O4)cc1C21c2ccccc2-c2cccc3cccc1c23 ; Cc1cc2c(cc1C)-c1cccc3c(-c4ccc5c(c4)C(c4ccccc4)(c4ccccc4)c4ccccc4-5)ccc(c13)O2]

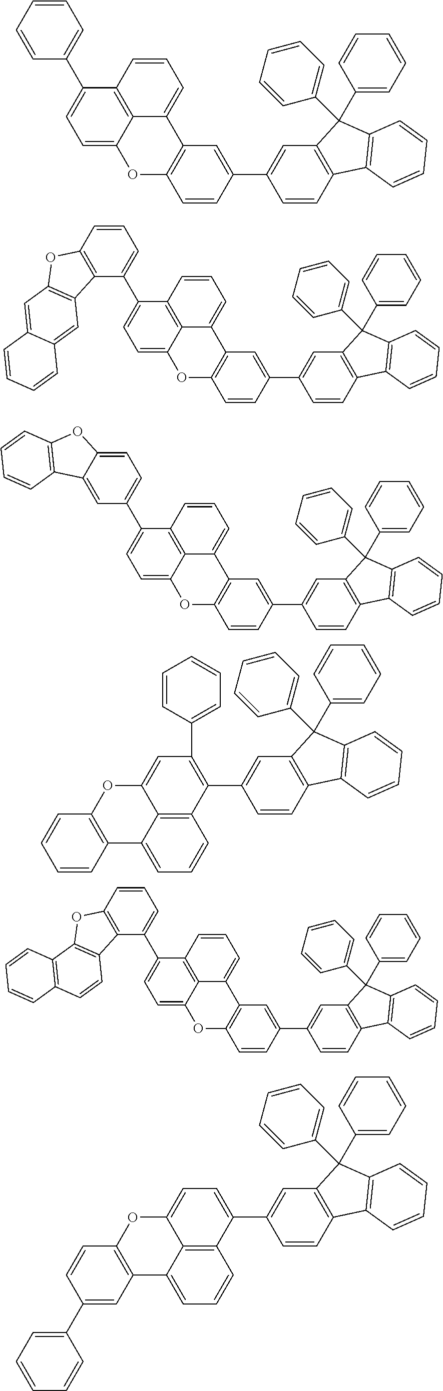

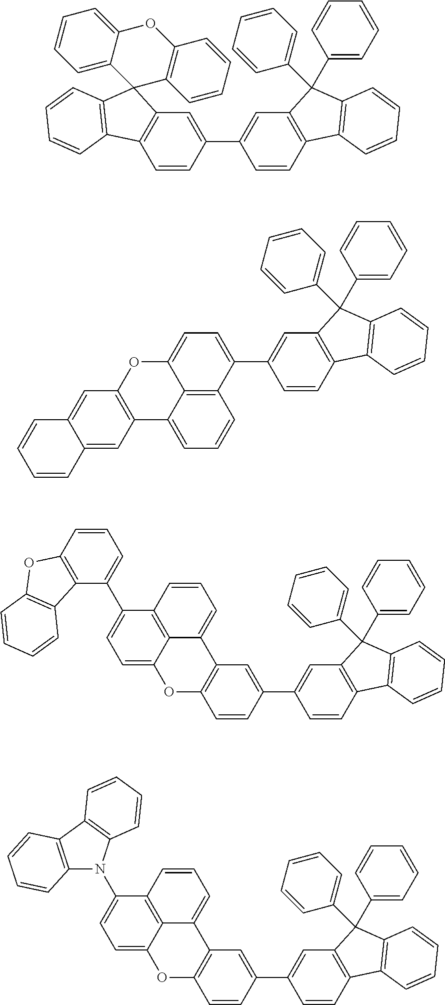 [SMILES: c1ccc(C2(c3ccccc3)c3ccccc3-c3ccc(-c4ccc5c(c4)C4(c6ccccc6Oc6ccccc64)c4ccccc4-5)cc32)cc1 ; c1ccc(C2(c3ccccc3)c3ccccc3-c3ccc(-c4ccc5c6c(cccc46)-c4cc6ccccc6cc4O5)cc32)cc1 ; c1ccc(C2(c3ccccc3)c3ccccc3-c3ccc(-c4ccc5c(c4)-c4cccc6c(-c7cccc8oc9ccccc9c78)ccc(c46)O5)cc32)cc1 ; c1ccc(C2(c3ccccc3)c3ccccc3-c3ccc(-c4ccc5c(c4)-c4cccc6c(-n7c8ccccc8c8ccccc87)ccc(c46)O5)cc32)cc1]

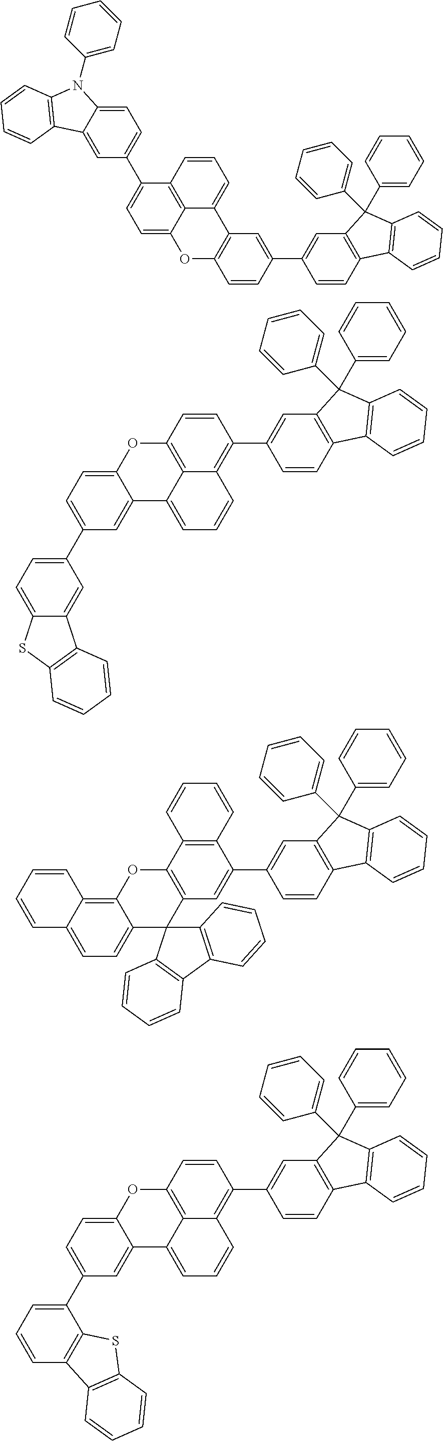

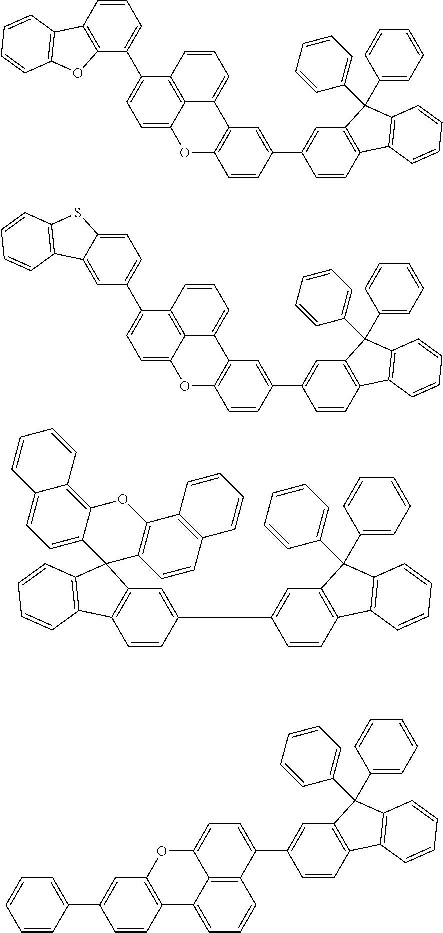 [SMILES: c1ccc(C2(c3ccccc3)c3ccccc3-c3ccc(-c4ccc5c(c4)-c4cccc6c(-c7cccc8c7oc7ccccc78)ccc(c46)O5)cc32)cc1 ; c1ccc(C2(c3ccccc3)c3ccccc3-c3ccc(-c4ccc5c(c4)-c4cccc6c(-c7ccc8sc9ccccc9c8c7)ccc(c46)O5)cc32)cc1 ; c1ccc(C2(c3ccccc3)c3ccccc3-c3ccc(-c4ccc5c(c4)C4(c6ccccc6-5)c5ccc6ccccc6c5Oc5c4ccc4ccccc54)cc32)cc1 ; c1ccc(-c2ccc3c(c2)Oc2ccc(-c4ccc5c(c4)C(c4ccccc4)(c4ccccc4)c4ccccc4-5)c4cccc-3c24)cc1]

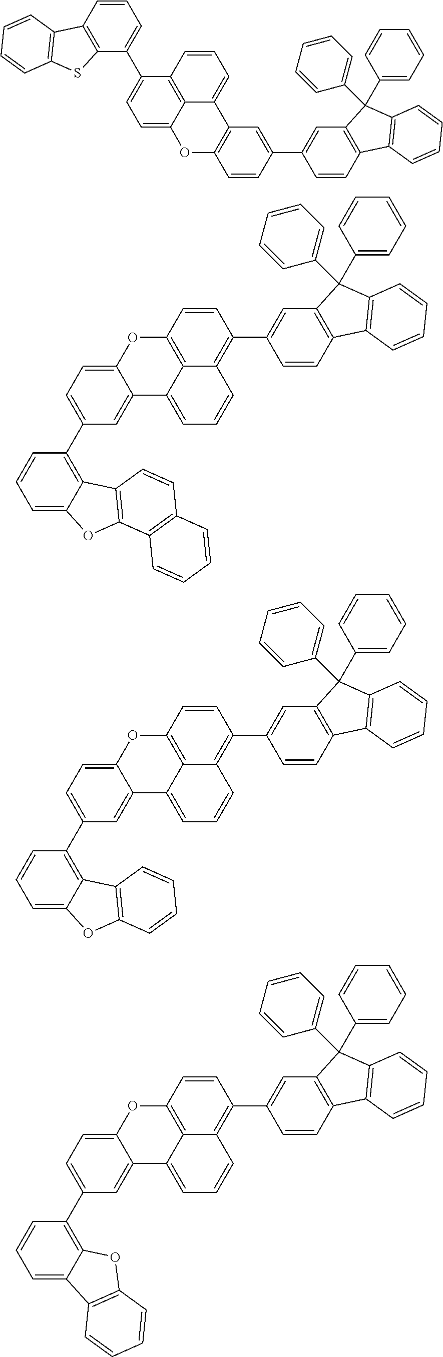

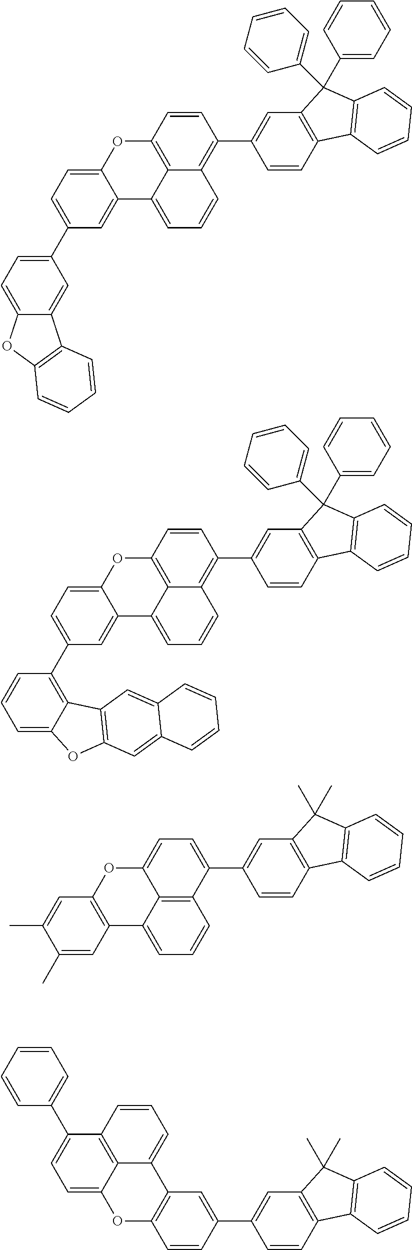 [SMILES: c1ccc(C2(c3ccccc3)c3ccccc3-c3ccc(-c4ccc5c6c(cccc46)-c4cc(-c6ccc7oc8ccccc8c7c6)ccc4O5)cc32)cc1 ; c1ccc(C2(c3ccccc3)c3ccccc3-c3ccc(-c4ccc5c6c(cccc46)-c4cc(-c6cccc7oc8cc9ccccc9cc8c67)ccc4O5)cc32)cc1 ; Cc1cc2c(cc1C)-c1cccc3c(-c4ccc5c(c4)C(C)(C)c4ccccc4-5)ccc(c13)O2 ; CC1(C)c2ccccc2-c2ccc(-c3ccc4c(c3)-c3cccc5c(-c6ccccc6)ccc(c35)O4)cc21]

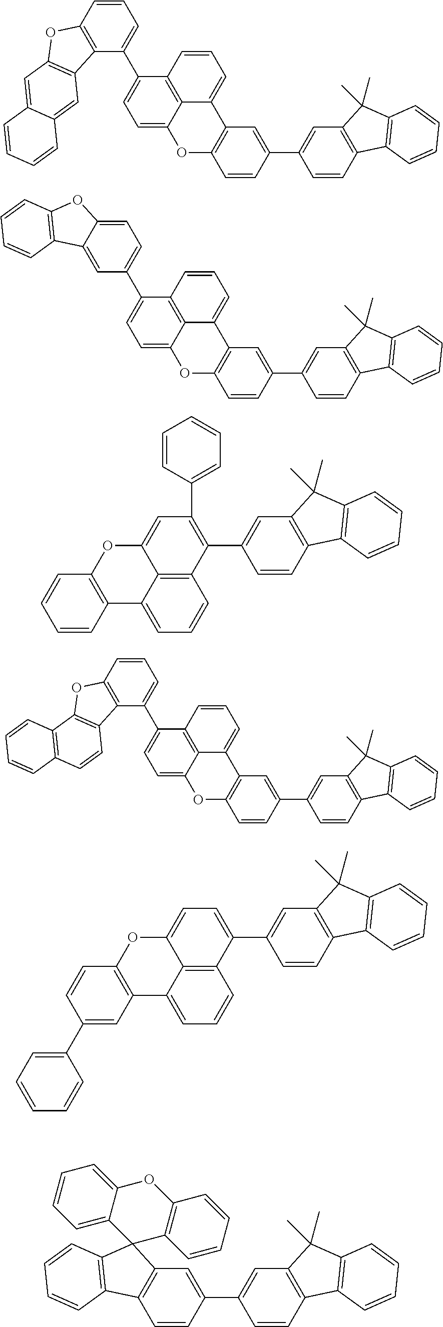

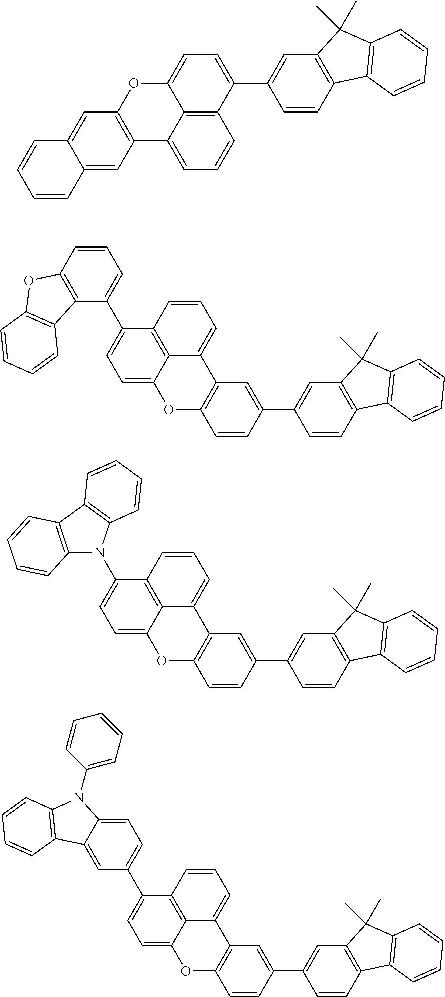 [SMILES: CC1(C)c2ccccc2-c2ccc(-c3ccc4c5c(cccc35)-c3cc5ccccc5cc3O4)cc21 ; CC1(C)c2ccccc2-c2ccc(-c3ccc4c(c3)-c3cccc5c(-c6cccc7oc8ccccc8c67)ccc(c35)O4)cc21 ; CC1(C)c2ccccc2-c2ccc(-c3ccc4c(c3)-c3cccc5c(-n6c7ccccc7c7ccccc76)ccc(c35)O4)cc21 ; CC1(C)c2ccccc2-c2ccc(-c3ccc4c(c3)-c3cccc5c(-c6ccc7c(c6)c6ccccc6n7-c6ccccc6)ccc(c35)O4)cc21]

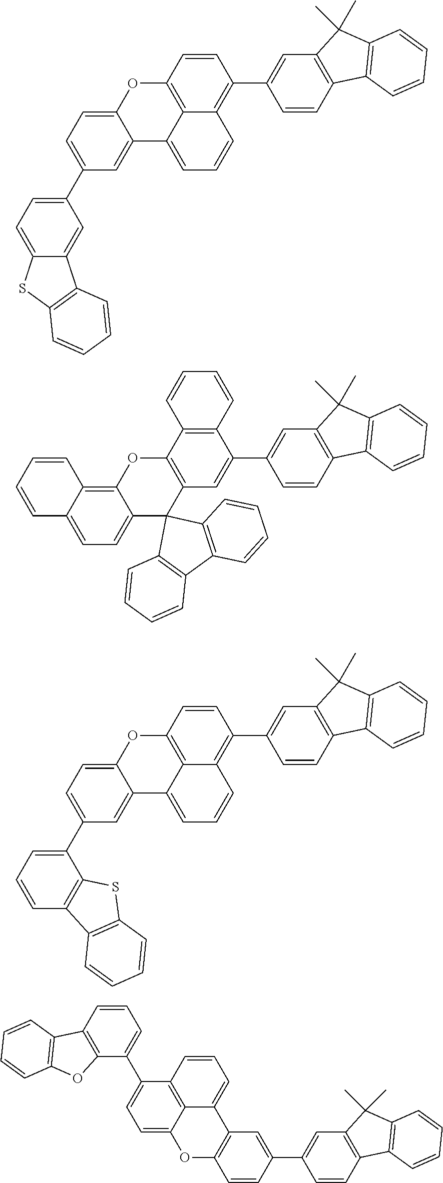

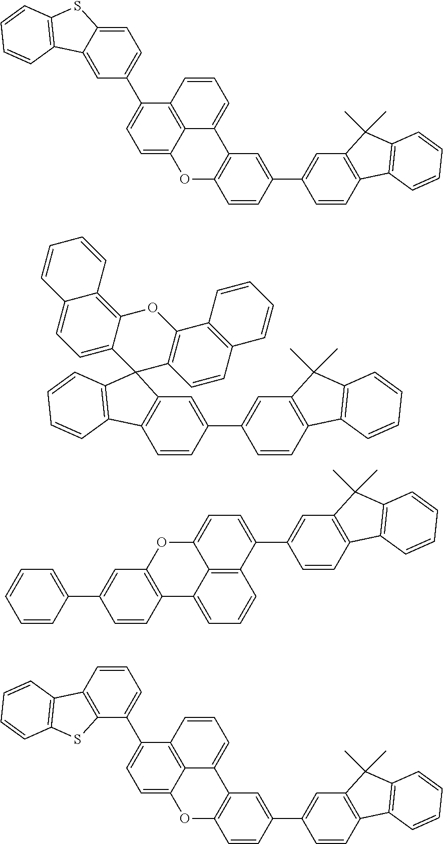

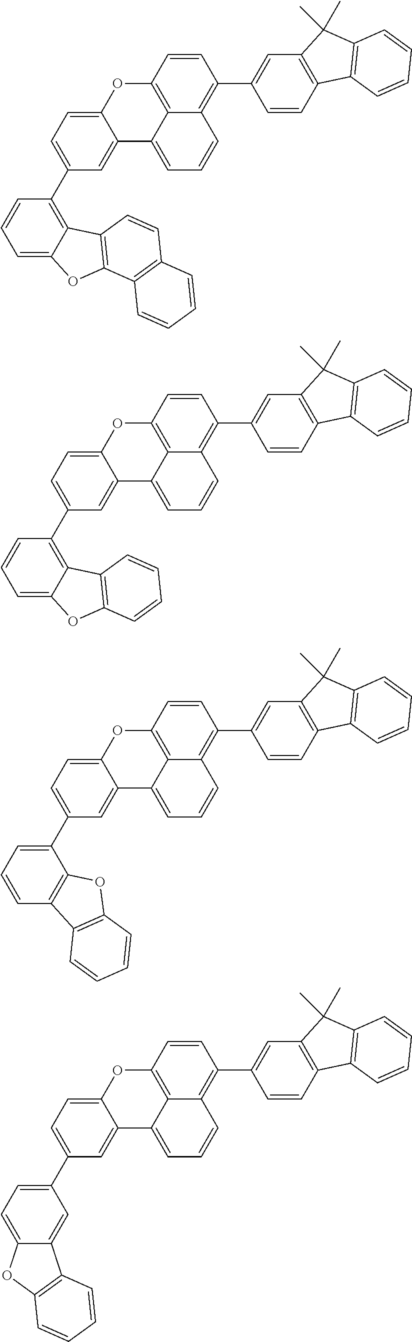 [SMILES: CC1(C)c2ccccc2-c2ccc(-c3ccc4c5c(cccc35)-c3cc(-c5cccc6oc7c8ccccc8ccc7c56)ccc3O4)cc21 ; CC1(C)c2ccccc2-c2ccc(-c3ccc4c5c(cccc35)-c3cc(-c5cccc6oc7ccccc7c56)ccc3O4)cc21 ; CC1(C)c2ccccc2-c2ccc(-c3ccc4c5c(cccc35)-c3cc(-c5cccc6c5oc5ccccc56)ccc3O4)cc21 ; CC1(C)c2ccccc2-c2ccc(-c3ccc4c5c(cccc35)-c3cc(-c5ccc6oc7ccccc7c6c5)ccc3O4)cc21]

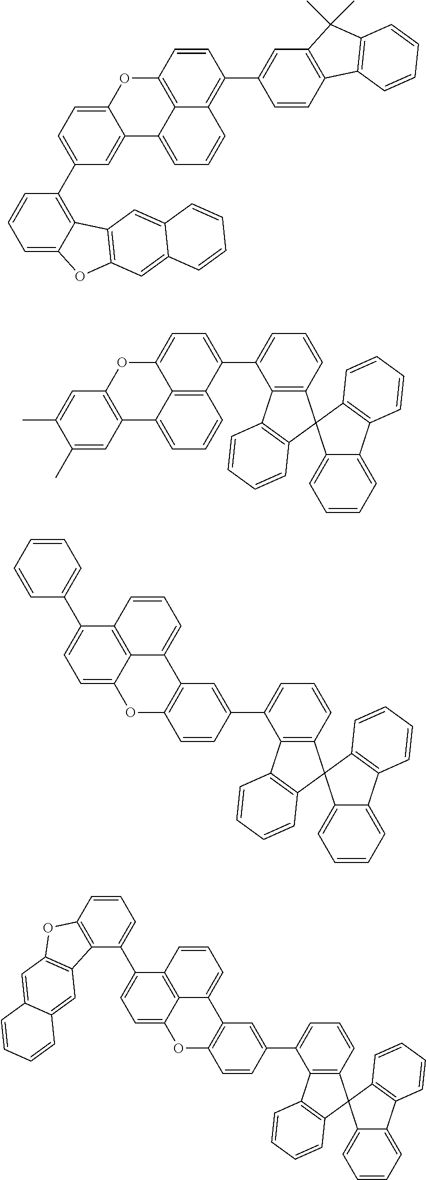 [SMILES: CC1(C)c2ccccc2-c2ccc(-c3ccc4c5c(cccc35)-c3cc(-c5cccc6oc7cc8ccccc8cc7c56)ccc3O4)cc21 ; Cc1cc2c(cc1C)-c1cccc3c(-c4cccc5c4-c4ccccc4C54c5ccccc5-c5ccccc54)ccc(c13)O2 ; c1ccc(-c2ccc3c4c(cccc24)-c2cc(-c4cccc5c4-c4ccccc4C54c5ccccc5-c5ccccc54)ccc2O3)cc1 ; c1ccc2c(c1)-c1ccccc1C21c2ccccc2-c2c(-c3ccc4c(c3)-c3cccc5c(-c6cccc7oc8cc9ccccc9cc8c67)ccc(c35)O4)cccc21]

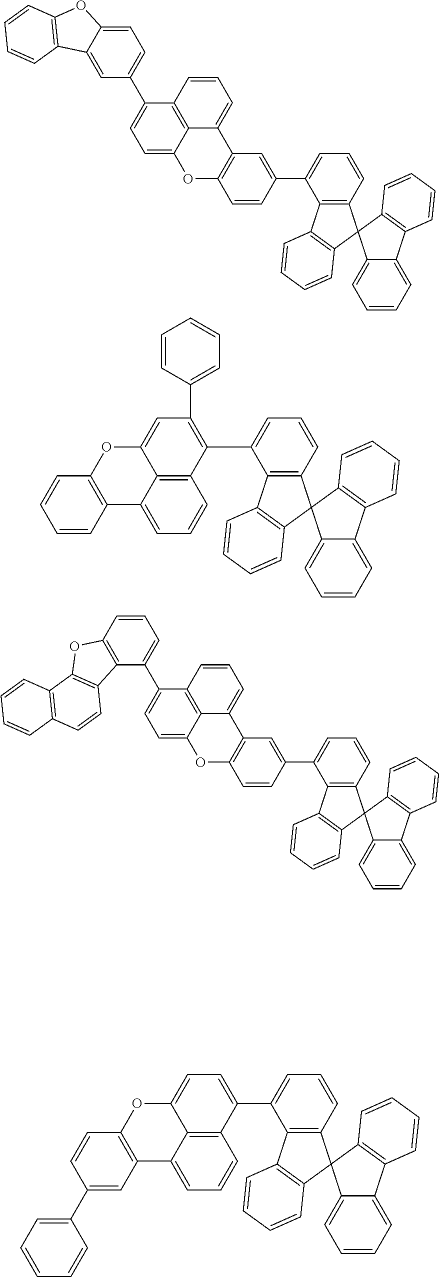 [SMILES: c1ccc2c(c1)-c1ccccc1C21c2ccccc2-c2c(-c3ccc4c(c3)-c3cccc5c(-c6ccc7oc8ccccc8c7c6)ccc(c35)O4)cccc21 ; c1ccc(-c2cc3c4c(cccc4c2-c2cccc4c2-c2ccccc2C42c4ccccc4-c4ccccc42)-c2ccccc2O3)cc1 ; c1ccc2c(c1)-c1ccccc1C21c2ccccc2-c2c(-c3ccc4c(c3)-c3cccc5c(-c6cccc7oc8c9ccccc9ccc8c67)ccc(c35)O4)cccc21 ; c1ccc(-c2ccc3c(c2)-c2cccc4c(-c5cccc6c5-c5ccccc5C65c6ccccc6-c6ccccc65)ccc(c24)O3)cc1]

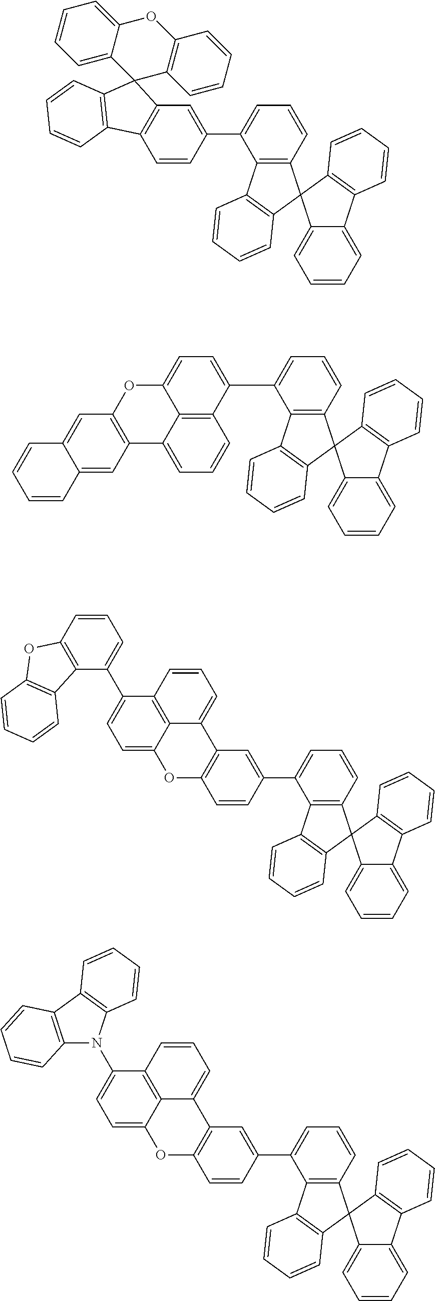 [SMILES: c1ccc2c(c1)Oc1ccccc1C21c2ccccc2-c2ccc(-c3cccc4c3-c3ccccc3C43c4ccccc4-c4ccccc43)cc21 ; c1ccc2c(c1)-c1ccccc1C21c2ccccc2-c2c(-c3ccc4c5c(cccc35)-c3cc5ccccc5cc3O4)cccc21 ; c1ccc2c(c1)-c1ccccc1C21c2ccccc2-c2c(-c3ccc4c(c3)-c3cccc5c(-c6cccc7oc8ccccc8c67)ccc(c35)O4)cccc21 ; c1ccc2c(c1)-c1ccccc1C21c2ccccc2-c2c(-c3ccc4c(c3)-c3cccc5c(-n6c7ccccc7c7ccccc76)ccc(c35)O4)cccc21]

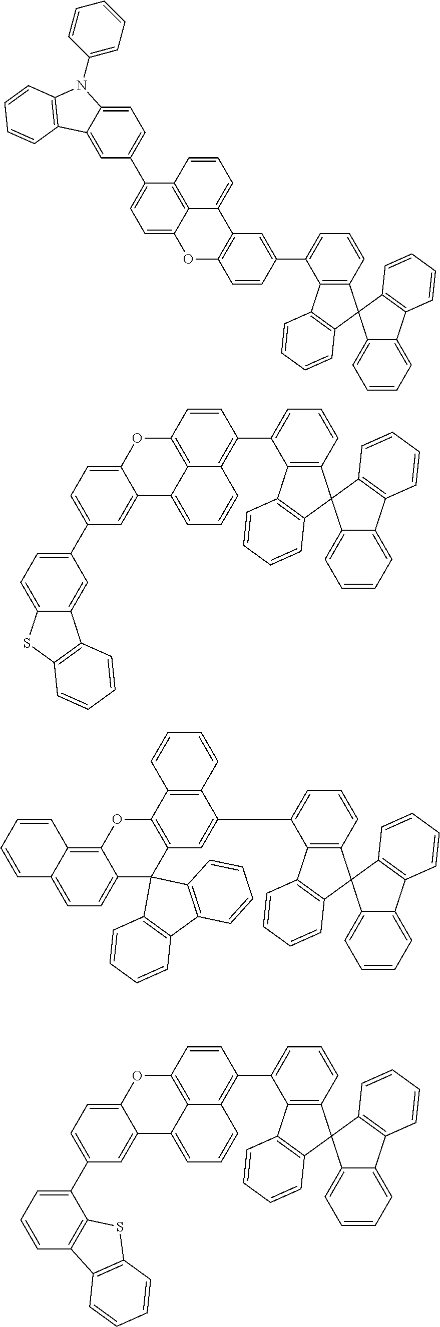

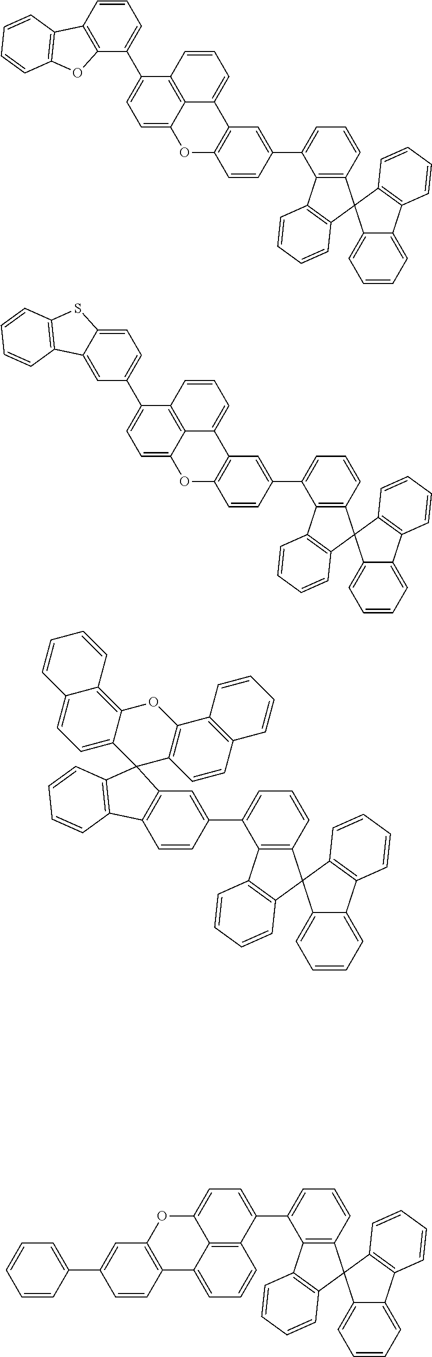 [SMILES: c1ccc2c(c1)-c1ccccc1C21c2ccccc2-c2c(-c3ccc4c(c3)-c3cccc5c(-c6cccc7c6oc6ccccc67)ccc(c35)O4)cccc21 ; c1ccc2c(c1)-c1ccccc1C21c2ccccc2-c2c(-c3ccc4c(c3)-c3cccc5c(-c6ccc7sc8ccccc8c7c6)ccc(c35)O4)cccc21 ; c1ccc2c(c1)-c1ccccc1C21c2ccccc2-c2c(-c3ccc4c(c3)C3(c5ccccc5-4)c4ccc5ccccc5c4Oc4c3ccc3ccccc43)cccc21 ; c1ccc(-c2ccc3c(c2)Oc2ccc(-c4cccc5c4-c4ccccc4C54c5ccccc5-c5ccccc54)c4cccc-3c24)cc1]

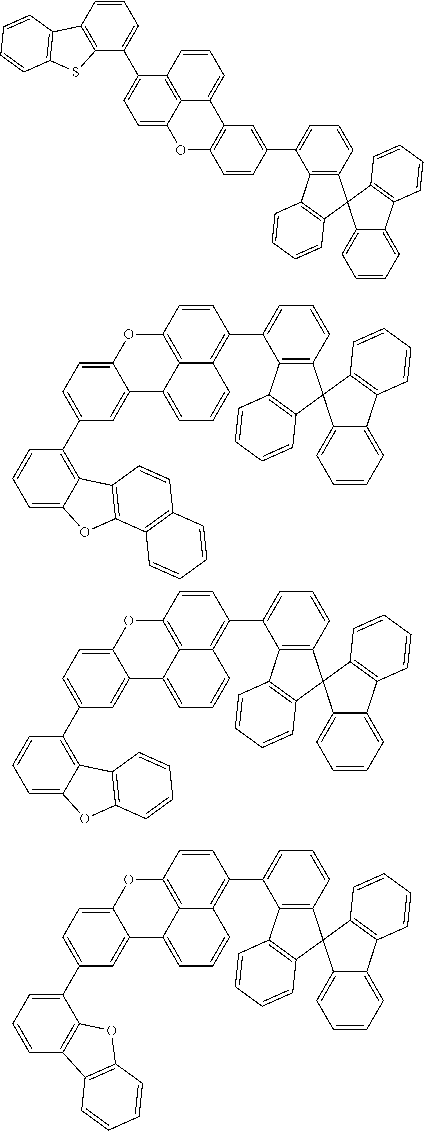

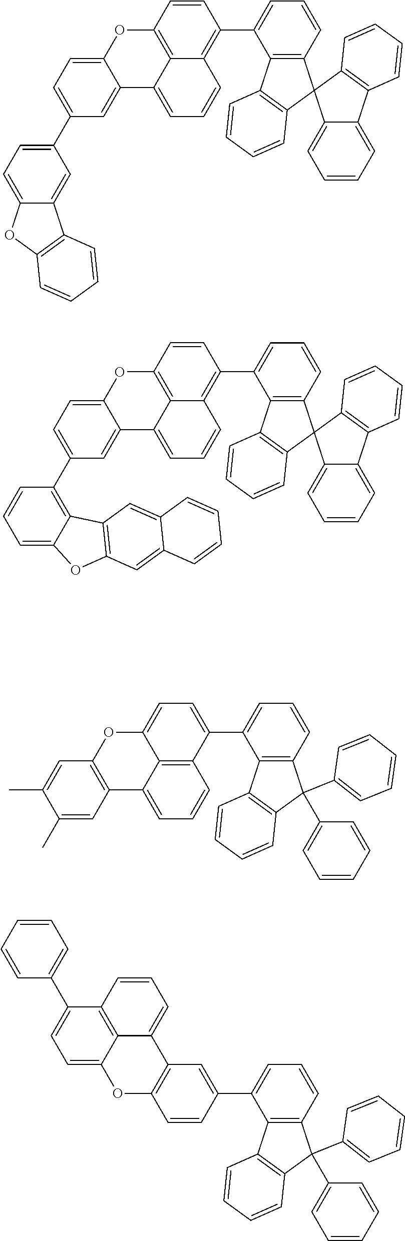 [SMILES: c1ccc2c(c1)-c1ccccc1C21c2ccccc2-c2c(-c3ccc4c5c(cccc35)-c3cc(-c5ccc6oc7ccccc7c6c5)ccc3O4)cccc21 ; c1ccc2c(c1)-c1ccccc1C21c2ccccc2-c2c(-c3ccc4c5c(cccc35)-c3cc(-c5cccc6oc7cc8ccccc8cc7c56)ccc3O4)cccc21 ; Cc1cc2c(cc1C)-c1cccc3c(-c4cccc5c4-c4ccccc4C5(c4ccccc4)c4ccccc4)ccc(c13)O2 ; c1ccc(-c2ccc3c4c(cccc24)-c2cc(-c4cccc5c4-c4ccccc4C5(c4ccccc4)c4ccccc4)ccc2O3)cc1]

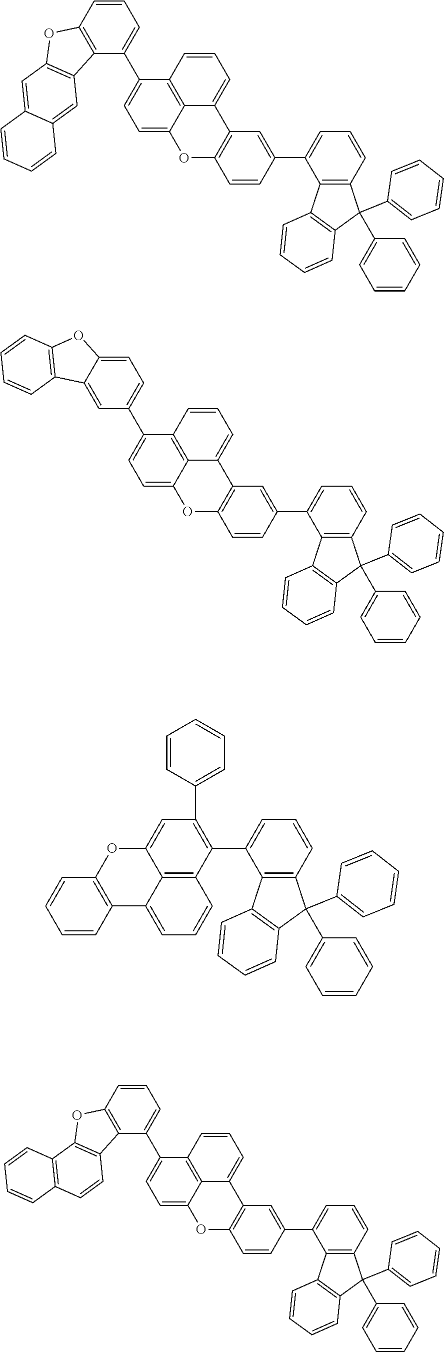 [SMILES: c1ccc(C2(c3ccccc3)c3ccccc3-c3c(-c4ccc5c(c4)-c4cccc6c(-c7cccc8oc9cc%10ccccc%10cc9c78)ccc(c46)O5)cccc32)cc1 ; c1ccc(C2(c3ccccc3)c3ccccc3-c3c(-c4ccc5c(c4)-c4cccc6c(-c7ccc8oc9ccccc9c8c7)ccc(c46)O5)cccc32)cc1 ; c1ccc(-c2cc3c4c(cccc4c2-c2cccc4c2-c2ccccc2C4(c2ccccc2)c2ccccc2)-c2ccccc2O3)cc1 ; c1ccc(C2(c3ccccc3)c3ccccc3-c3c(-c4ccc5c(c4)-c4cccc6c(-c7cccc8oc9c%10ccccc%10ccc9c78)ccc(c46)O5)cccc32)cc1]

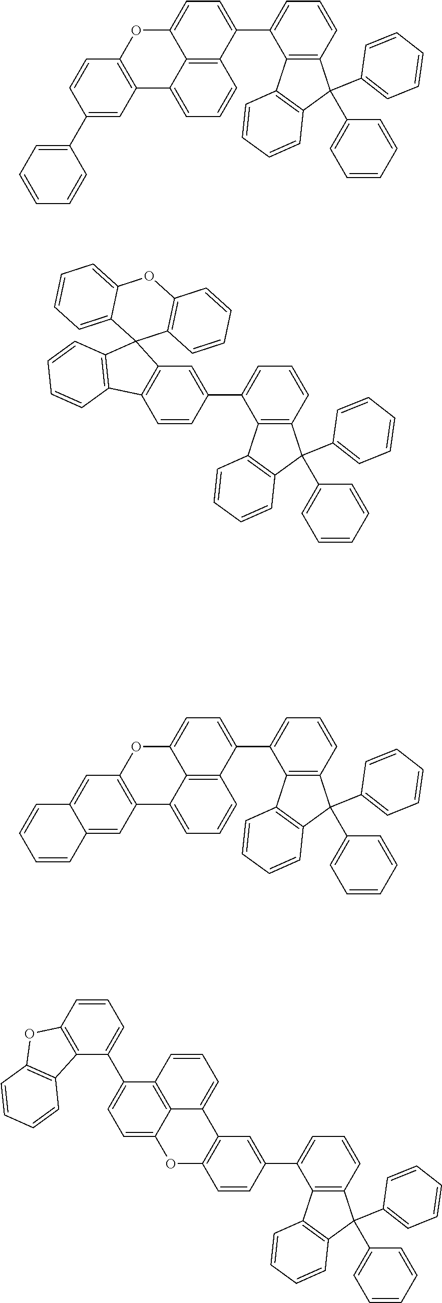 [SMILES: c1ccc(-c2ccc3c(c2)-c2cccc4c(-c5cccc6c5-c5ccccc5C6(c5ccccc5)c5ccccc5)ccc(c24)O3)cc1 ; c1ccc(C2(c3ccccc3)c3ccccc3-c3c(-c4ccc5c(c4)C4(c6ccccc6Oc6ccccc64)c4ccccc4-5)cccc32)cc1 ; c1ccc(C2(c3ccccc3)c3ccccc3-c3c(-c4ccc5c6c(cccc46)-c4cc6ccccc6cc4O5)cccc32)cc1 ; c1ccc(C2(c3ccccc3)c3ccccc3-c3c(-c4ccc5c(c4)-c4cccc6c(-c7cccc8oc9ccccc9c78)ccc(c46)O5)cccc32)cc1]

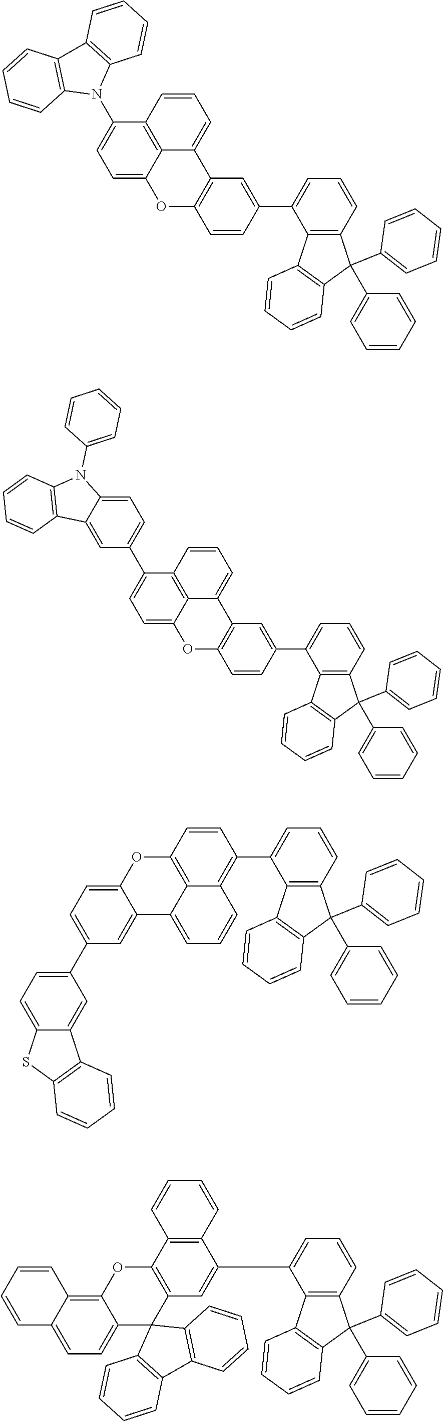 [SMILES: c1ccc(C2(c3ccccc3)c3ccccc3-c3c(-c4ccc5c(c4)-c4cccc6c(-n7c8ccccc8c8ccccc87)ccc(c46)O5)cccc32)cc1 ; c1ccc(-n2c3ccccc3c3cc(-c4ccc5c6c(cccc46)-c4cc(-c6cccc7c6-c6ccccc6C7(c6ccccc6)c6ccccc6)ccc4O5)ccc32)cc1 ; c1ccc(C2(c3ccccc3)c3ccccc3-c3c(-c4ccc5c6c(cccc46)-c4cc(-c6ccc7sc8ccccc8c7c6)ccc4O5)cccc32)cc1 ; c1ccc(C2(c3ccccc3)c3ccccc3-c3c(-c4cc5c(c6ccccc46)Oc4c(ccc6ccccc46)C54c5ccccc5-c5ccccc54)cccc32)cc1]

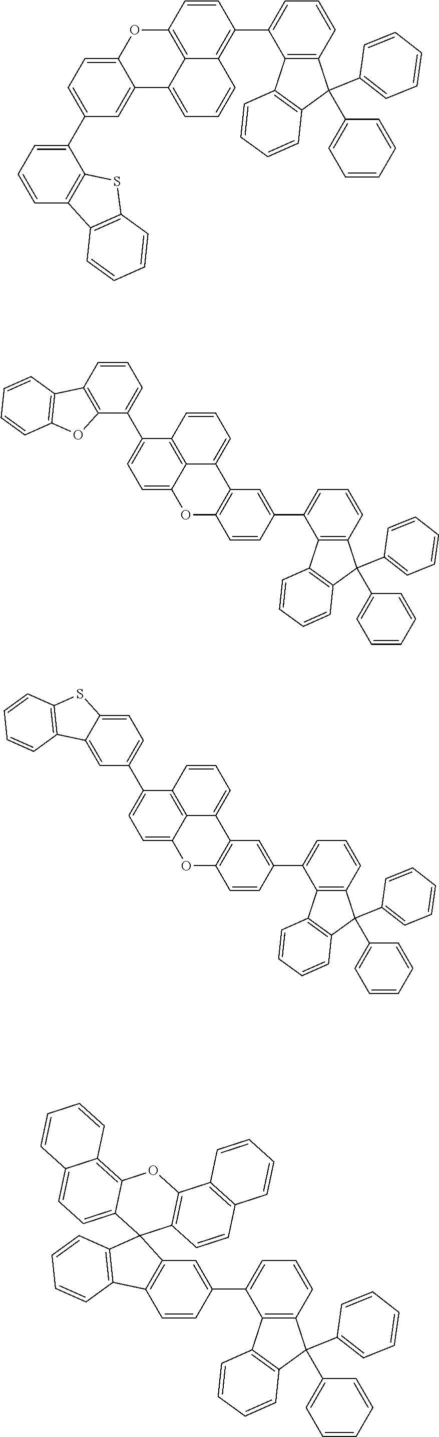 [SMILES: c1ccc(C2(c3ccccc3)c3ccccc3-c3c(-c4ccc5c6c(cccc46)-c4cc(-c6cccc7c6sc6ccccc67)ccc4O5)cccc32)cc1 ; c1ccc(C2(c3ccccc3)c3ccccc3-c3c(-c4ccc5c(c4)-c4cccc6c(-c7cccc8c7oc7ccccc78)ccc(c46)O5)cccc32)cc1 ; c1ccc(C2(c3ccccc3)c3ccccc3-c3c(-c4ccc5c(c4)-c4cccc6c(-c7ccc8sc9ccccc9c8c7)ccc(c46)O5)cccc32)cc1 ; c1ccc(C2(c3ccccc3)c3ccccc3-c3c(-c4ccc5c(c4)C4(c6ccccc6-5)c5ccc6ccccc6c5Oc5c4ccc4ccccc54)cccc32)cc1]

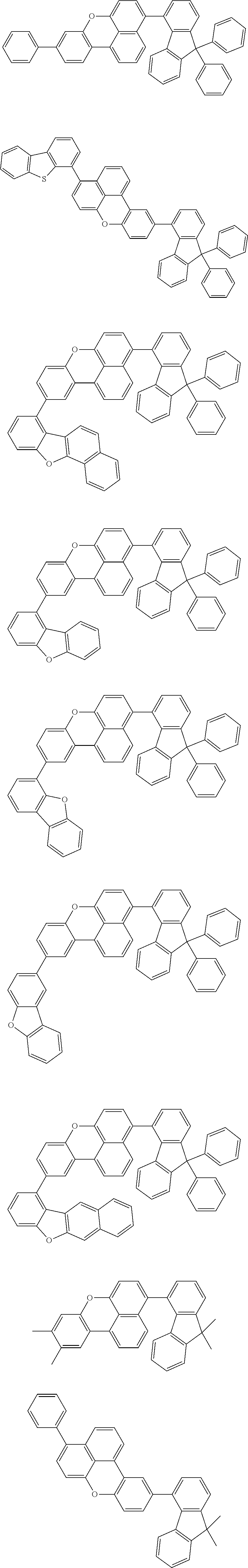

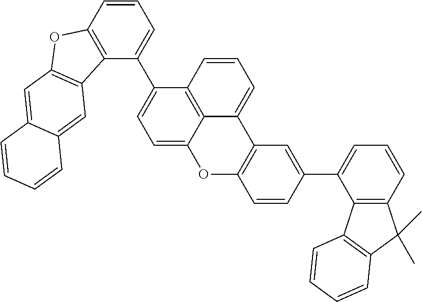 [SMILES: CC1(C)c2ccccc2-c2c(-c3ccc4c(c3)-c3cccc5c(-c6cccc7oc8cc9ccccc9cc8c67)ccc(c35)O4)cccc21]

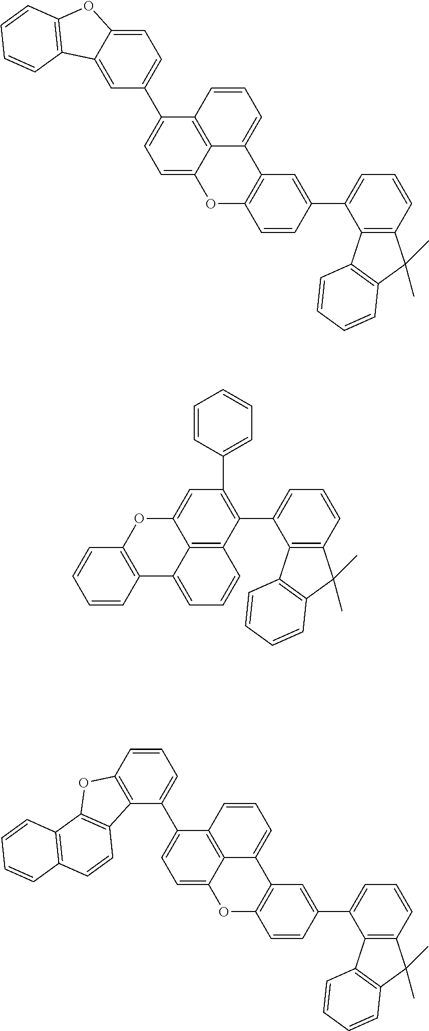 [SMILES: CC1(C)c2ccccc2-c2c(-c3ccc4c(c3)-c3cccc5c(-c6ccc7oc8ccccc8c7c6)ccc(c35)O4)cccc21 ; CC1(C)c2ccccc2-c2c(-c3c(-c4ccccc4)cc4c5c(cccc35)-c3ccccc3O4)cccc21 ; CC1(C)c2ccccc2-c2c(-c3ccc4c(c3)-c3cccc5c(-c6cccc7oc8c9ccccc9ccc8c67)ccc(c35)O4)cccc21]

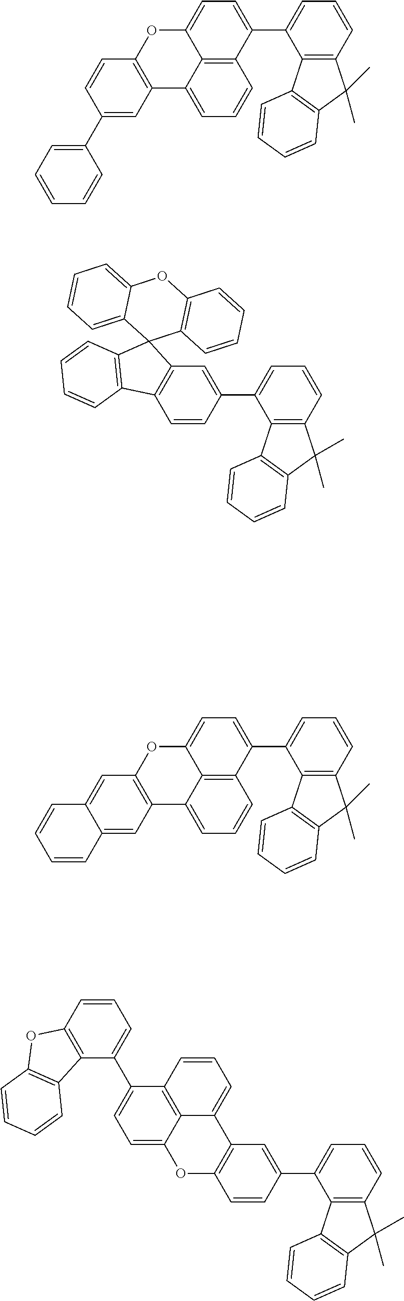 [SMILES: CC1(C)c2ccccc2-c2c(-c3ccc4c5c(cccc35)-c3cc(-c5ccccc5)ccc3O4)cccc21 ; CC1(C)c2ccccc2-c2c(-c3ccc4c(c3)C3(c5ccccc5Oc5ccccc53)c3ccccc3-4)cccc21 ; CC1(C)c2ccccc2-c2c(-c3ccc4c5c(cccc35)-c3cc5ccccc5cc3O4)cccc21 ; CC1(C)c2ccccc2-c2c(-c3ccc4c(c3)-c3cccc5c(-c6cccc7oc8ccccc8c67)ccc(c35)O4)cccc21]

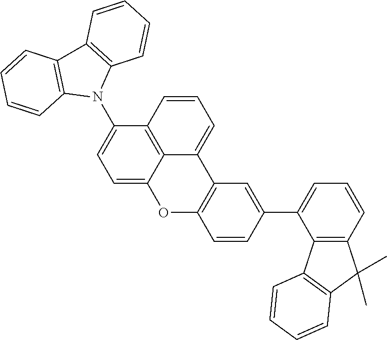 [SMILES: CC1(C)c2ccccc2-c2c(-c3ccc4c(c3)-c3cccc5c(-n6c7ccccc7c7ccccc76)ccc(c35)O4)cccc21]

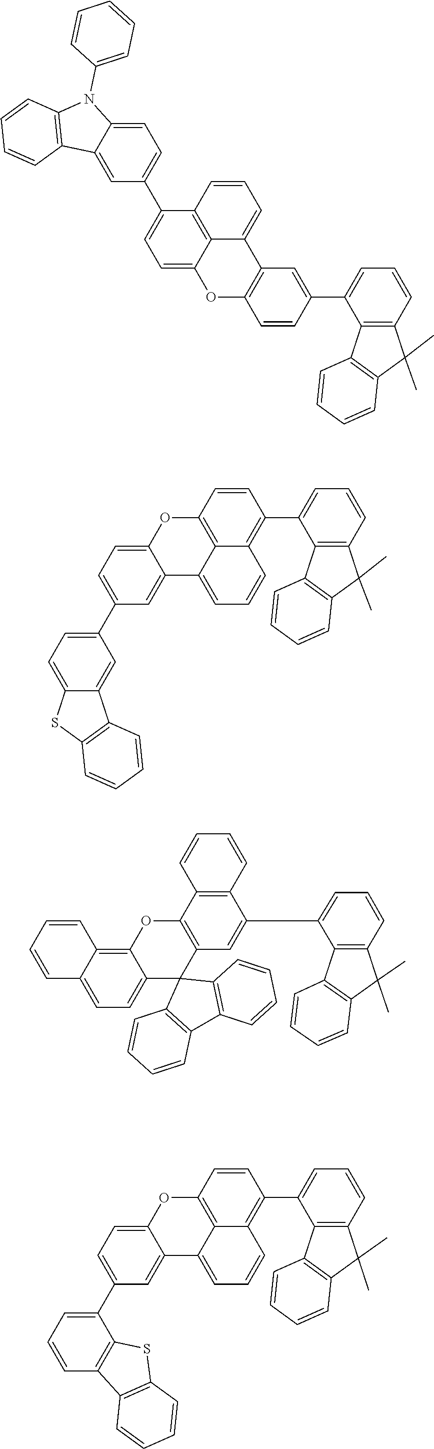 [SMILES: CC1(C)c2ccccc2-c2c(-c3ccc4c(c3)-c3cccc5c(-c6ccc7c(c6)c6ccccc6n7-c6ccccc6)ccc(c35)O4)cccc21 ; CC1(C)c2ccccc2-c2c(-c3ccc4c5c(cccc35)-c3cc(-c5ccc6sc7ccccc7c6c5)ccc3O4)cccc21 ; CC1(C)c2ccccc2-c2c(-c3cc4c(c5ccccc35)Oc3c(ccc5ccccc35)C43c4ccccc4-c4ccccc43)cccc21 ; CC1(C)c2ccccc2-c2c(-c3ccc4c5c(cccc35)-c3cc(-c5cccc6c5sc5ccccc56)ccc3O4)cccc21]

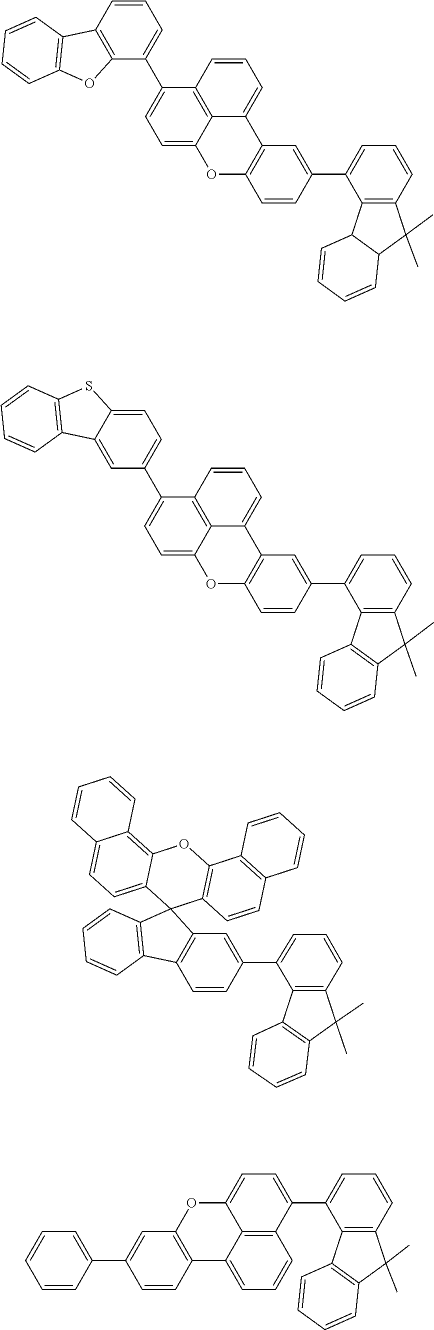 [SMILES: CC1(C)c2cccc(-c3ccc4c(c3)-c3cccc5c(-c6cccc7c6oc6ccccc67)ccc(c35)O4)c2C2C=CC=CC21 ; CC1(C)c2ccccc2-c2c(-c3ccc4c(c3)-c3cccc5c(-c6ccc7sc8ccccc8c7c6)ccc(c35)O4)cccc21 ; CC1(C)c2ccccc2-c2c(-c3ccc4c(c3)C3(c5ccccc5-4)c4ccc5ccccc5c4Oc4c3ccc3ccccc43)cccc21 ; CC1(C)c2ccccc2-c2c(-c3ccc4c5c(cccc35)-c3ccc(-c5ccccc5)cc3O4)cccc21]

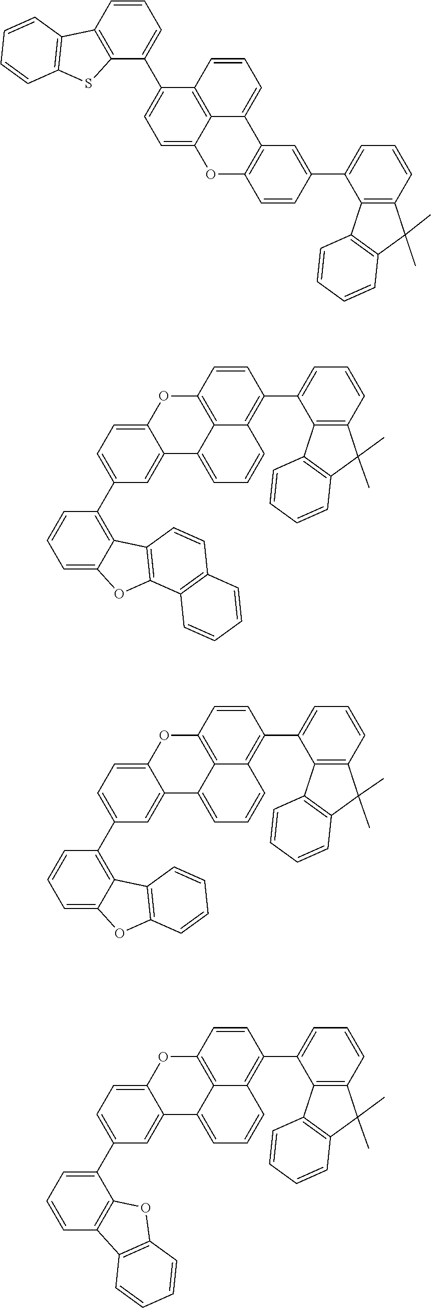 [SMILES: CC1(C)c2ccccc2-c2c(-c3ccc4c(c3)-c3cccc5c(-c6cccc7c6sc6ccccc67)ccc(c35)O4)cccc21 ; CC1(C)c2ccccc2-c2c(-c3ccc4c5c(cccc35)-c3cc(-c5cccc6oc7c8ccccc8ccc7c56)ccc3O4)cccc21 ; CC1(C)c2ccccc2-c2c(-c3ccc4c5c(cccc35)-c3cc(-c5cccc6oc7ccccc7c56)ccc3O4)cccc21 ; CC1(C)c2ccccc2-c2c(-c3ccc4c5c(cccc35)-c3cc(-c5cccc6c5oc5ccccc56)ccc3O4)cccc21]

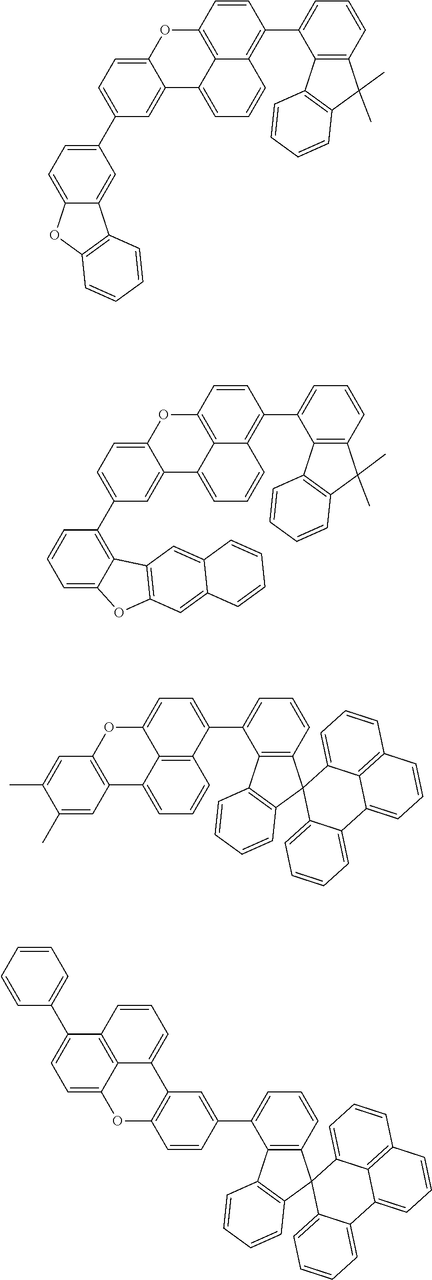

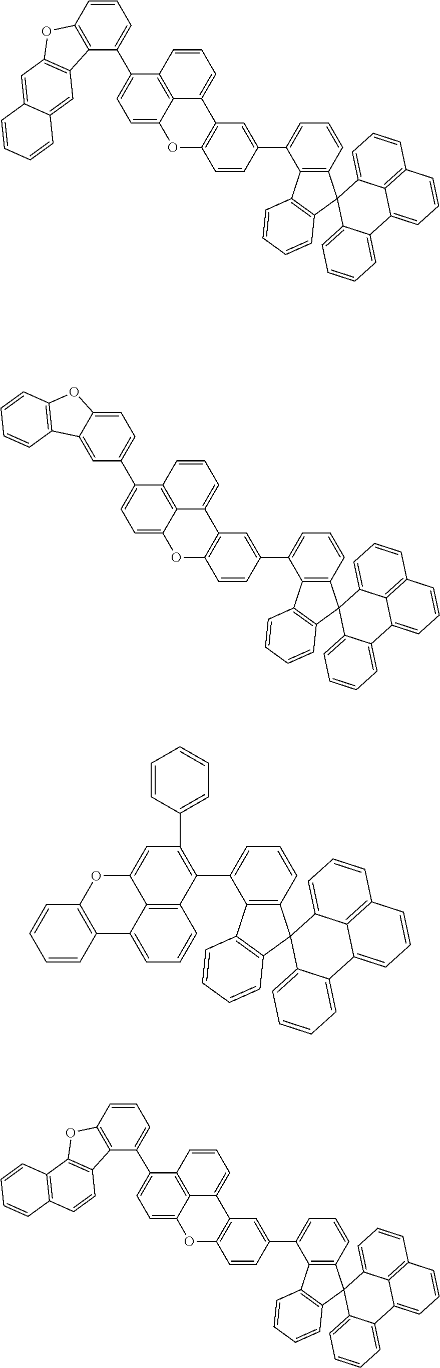 [SMILES: c1ccc2c(c1)-c1c(-c3ccc4c(c3)-c3cccc5c(-c6cccc7oc8cc9ccccc9cc8c67)ccc(c35)O4)cccc1C21c2ccccc2-c2cccc3cccc1c23 ; c1ccc2c(c1)-c1c(-c3ccc4c(c3)-c3cccc5c(-c6ccc7oc8ccccc8c7c6)ccc(c35)O4)cccc1C21c2ccccc2-c2cccc3cccc1c23 ; c1ccc(-c2cc3c4c(cccc4c2-c2cccc4c2-c2ccccc2C42c4ccccc4-c4cccc5cccc2c45)-c2ccccc2O3)cc1 ; c1ccc2c(c1)-c1c(-c3ccc4c(c3)-c3cccc5c(-c6cccc7oc8c9ccccc9ccc8c67)ccc(c35)O4)cccc1C21c2ccccc2-c2cccc3cccc1c23]

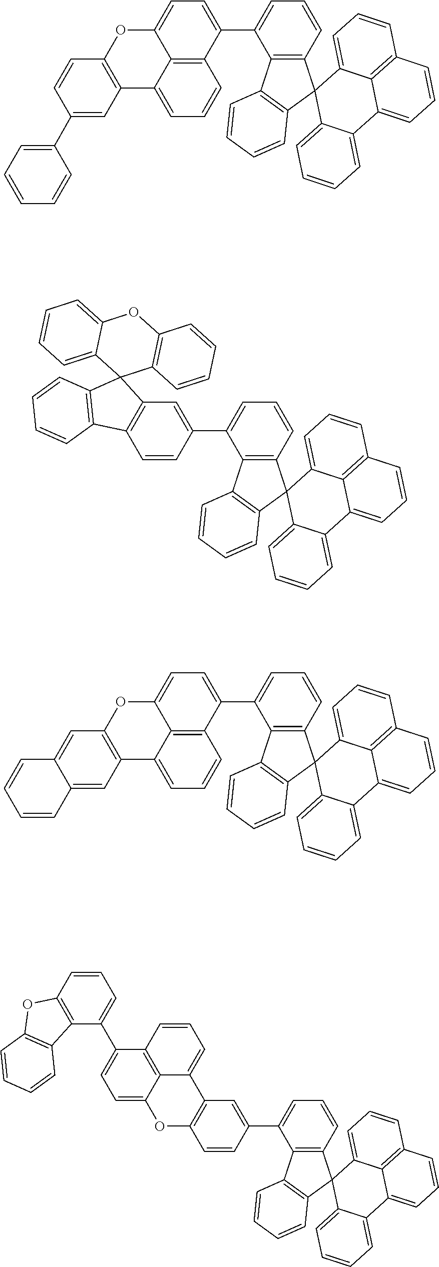 [SMILES: c1ccc(-c2ccc3c(c2)-c2cccc4c(-c5cccc6c5-c5ccccc5C65c6ccccc6-c6cccc7cccc5c67)ccc(c24)O3)cc1 ; c1ccc2c(c1)Oc1ccccc1C21c2ccccc2-c2ccc(-c3cccc4c3-c3ccccc3C43c4ccccc4-c4cccc5cccc3c45)cc21 ; c1ccc2c(c1)-c1c(-c3ccc4c5c(cccc35)-c3cc5ccccc5cc3O4)cccc1C21c2ccccc2-c2cccc3cccc1c23 ; c1ccc2c(c1)-c1c(-c3ccc4c(c3)-c3cccc5c(-c6cccc7oc8ccccc8c67)ccc(c35)O4)cccc1C21c2ccccc2-c2cccc3cccc1c23]

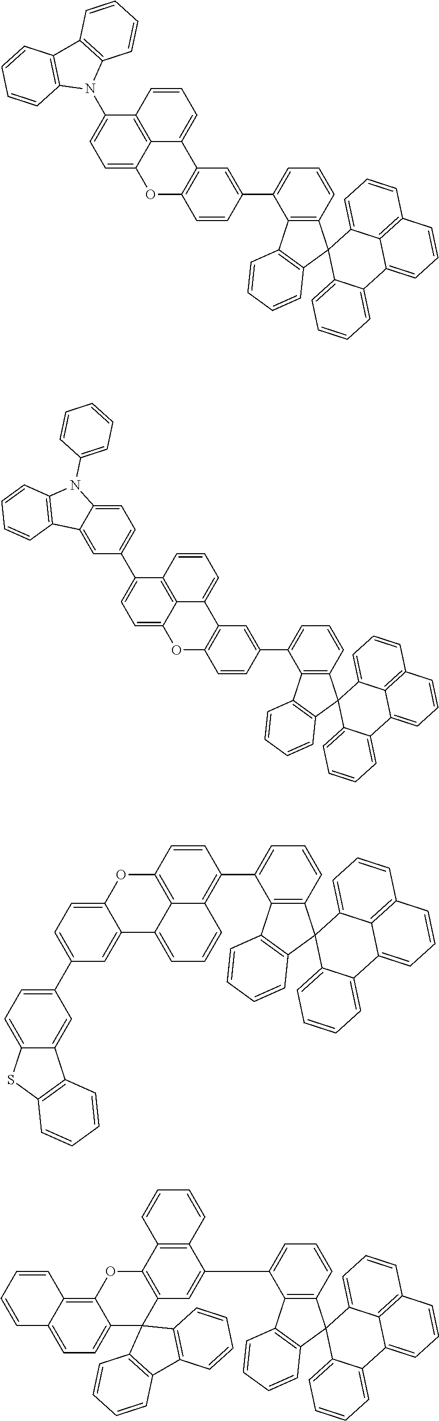 [SMILES: c1ccc2c(c1)-c1c(-c3ccc4c(c3)-c3cccc5c(-n6c7ccccc7c7ccccc76)ccc(c35)O4)cccc1C21c2ccccc2-c2cccc3cccc1c23 ; c1ccc(-n2c3ccccc3c3cc(-c4ccc5c6c(cccc46)-c4cc(-c6cccc7c6-c6ccccc6C76c7ccccc7-c7cccc8cccc6c78)ccc4O5)ccc32)cc1 ; c1ccc2c(c1)-c1c(-c3ccc4c5c(cccc35)-c3cc(-c5ccc6sc7ccccc7c6c5)ccc3O4)cccc1C21c2ccccc2-c2cccc3cccc1c23 ; c1ccc2c(c1)-c1ccccc1C21c2ccc3ccccc3c2Oc2c1cc(-c1cccc3c1-c1ccccc1C31c3ccccc3-c3cccc4cccc1c34)c1ccccc21]

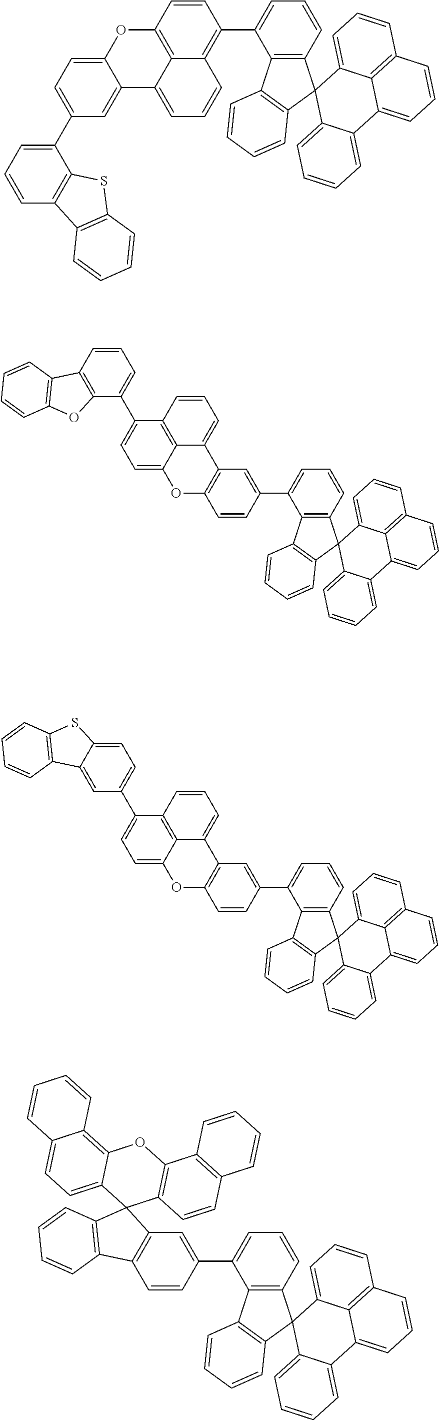 [SMILES: c1ccc2c(c1)-c1c(-c3ccc4c5c(cccc35)-c3cc(-c5cccc6c5sc5ccccc56)ccc3O4)cccc1C21c2ccccc2-c2cccc3cccc1c23 ; c1ccc2c(c1)-c1c(-c3ccc4c(c3)-c3cccc5c(-c6cccc7c6oc6ccccc67)ccc(c35)O4)cccc1C21c2ccccc2-c2cccc3cccc1c23 ; c1ccc2c(c1)-c1c(-c3ccc4c(c3)-c3cccc5c(-c6ccc7sc8ccccc8c7c6)ccc(c35)O4)cccc1C21c2ccccc2-c2cccc3cccc1c23 ; c1ccc2c(c1)-c1ccc(-c3cccc4c3-c3ccccc3C43c4ccccc4-c4cccc5cccc3c45)cc1C21c2ccc3ccccc3c2Oc2c1ccc1ccccc21]

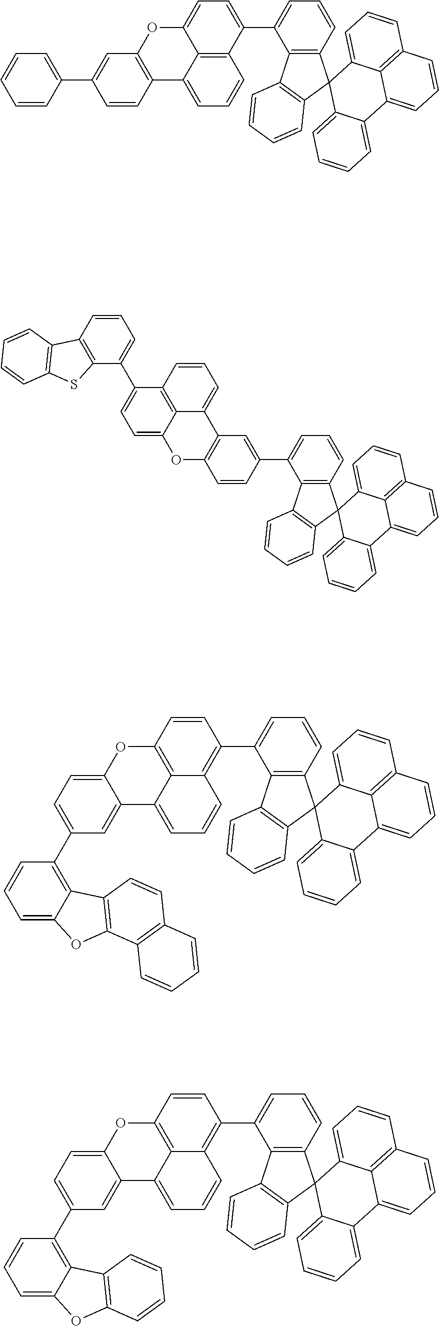 [SMILES: c1ccc(-c2ccc3c(c2)Oc2ccc(-c4cccc5c4-c4ccccc4C54c5ccccc5-c5cccc6cccc4c56)c4cccc-3c24)cc1 ; c1ccc2c(c1)-c1c(-c3ccc4c(c3)-c3cccc5c(-c6cccc7c6sc6ccccc67)ccc(c35)O4)cccc1C21c2ccccc2-c2cccc3cccc1c23 ; c1ccc2c(c1)-c1c(-c3ccc4c5c(cccc35)-c3cc(-c5cccc6oc7c8ccccc8ccc7c56)ccc3O4)cccc1C21c2ccccc2-c2cccc3cccc1c23 ; c1ccc2c(c1)-c1c(-c3ccc4c5c(cccc35)-c3cc(-c5cccc6oc7ccccc7c56)ccc3O4)cccc1C21c2ccccc2-c2cccc3cccc1c23]

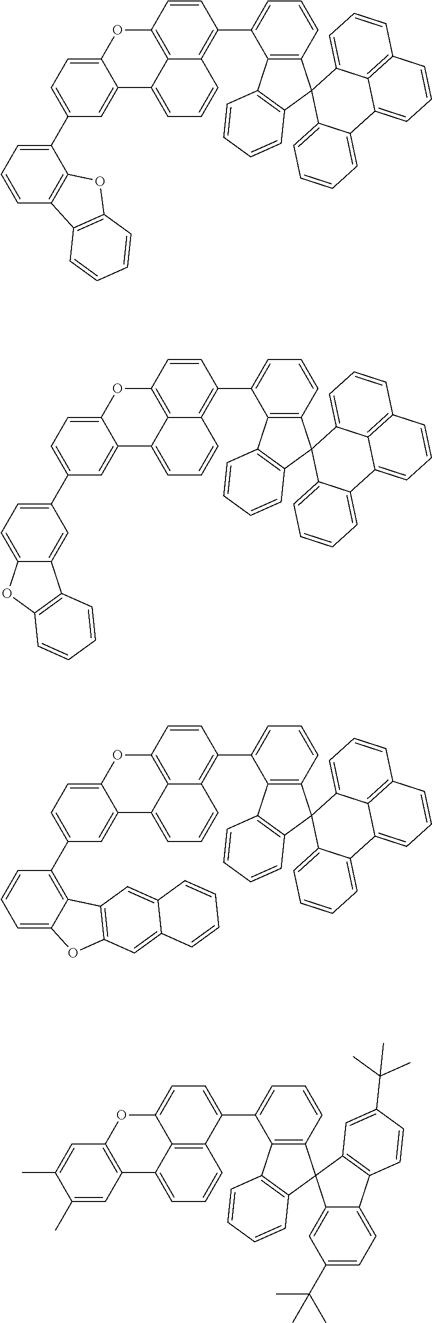 [SMILES: c1ccc2c(c1)-c1c(-c3ccc4c5c(cccc35)-c3cc(-c5cccc6c5oc5ccccc56)ccc3O4)cccc1C21c2ccccc2-c2cccc3cccc1c23 ; c1ccc2c(c1)-c1c(-c3ccc4c5c(cccc35)-c3cc(-c5ccc6oc7ccccc7c6c5)ccc3O4)cccc1C21c2ccccc2-c2cccc3cccc1c23 ; c1ccc2c(c1)-c1c(-c3ccc4c5c(cccc35)-c3cc(-c5cccc6oc7cc8ccccc8cc7c56)ccc3O4)cccc1C21c2ccccc2-c2cccc3cccc1c23 ; Cc1cc2c(cc1C)-c1cccc3c(-c4cccc5c4-c4ccccc4C54c5cc(C(C)(C)C)ccc5-c5ccc(C(C)(C)C)cc54)ccc(c13)O2]

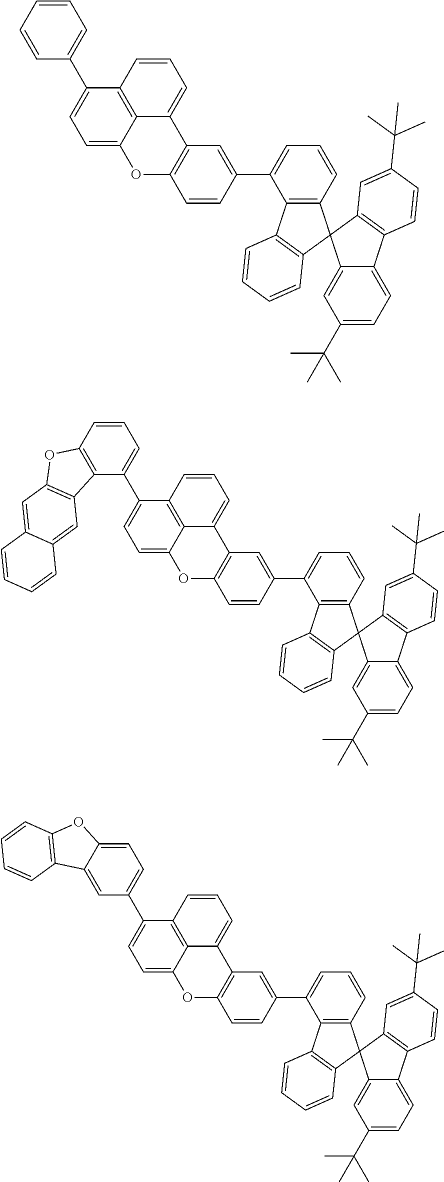 [SMILES: CC(C)(C)c1ccc2c(c1)C1(c3cc(C(C)(C)C)ccc3-2)c2ccccc2-c2c(-c3ccc4c(c3)-c3cccc5c(-c6ccccc6)ccc(c35)O4)cccc21 ; CC(C)(C)c1ccc2c(c1)C1(c3cc(C(C)(C)C)ccc3-2)c2ccccc2-c2c(-c3ccc4c(c3)-c3cccc5c(-c6cccc7oc8cc9ccccc9cc8c67)ccc(c35)O4)cccc21 ; CC(C)(C)c1ccc2c(c1)C1(c3cc(C(C)(C)C)ccc3-2)c2ccccc2-c2c(-c3ccc4c(c3)-c3cccc5c(-c6ccc7oc8ccccc8c7c6)ccc(c35)O4)cccc21]

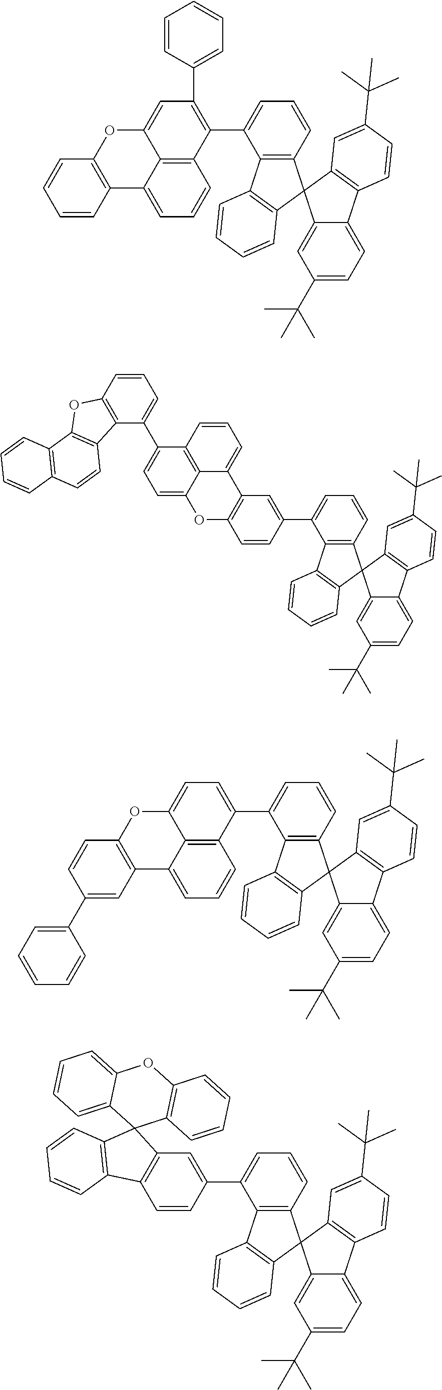 [SMILES: CC(C)(C)c1ccc2c(c1)C1(c3cc(C(C)(C)C)ccc3-2)c2ccccc2-c2c(-c3c(-c4ccccc4)cc4c5c(cccc35)-c3ccccc3O4)cccc21 ; CC(C)(C)c1ccc2c(c1)C1(c3cc(C(C)(C)C)ccc3-2)c2ccccc2-c2c(-c3ccc4c(c3)-c3cccc5c(-c6cccc7oc8c9ccccc9ccc8c67)ccc(c35)O4)cccc21 ; CC(C)(C)c1ccc2c(c1)C1(c3cc(C(C)(C)C)ccc3-2)c2ccccc2-c2c(-c3ccc4c5c(cccc35)-c3cc(-c5ccccc5)ccc3O4)cccc21 ; CC(C)(C)c1ccc2c(c1)C1(c3cc(C(C)(C)C)ccc3-2)c2ccccc2-c2c(-c3ccc4c(c3)C3(c5ccccc5Oc5ccccc53)c3ccccc3-4)cccc21]

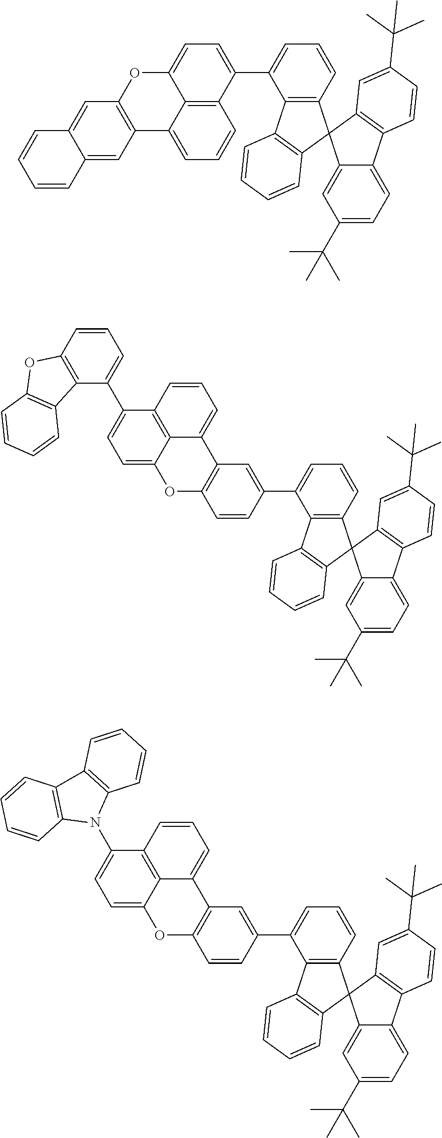 [SMILES: CC(C)(C)c1ccc2c(c1)C1(c3cc(C(C)(C)C)ccc3-2)c2ccccc2-c2c(-c3ccc4c5c(cccc35)-c3cc5ccccc5cc3O4)cccc21 ; CC(C)(C)c1ccc2c(c1)C1(c3cc(C(C)(C)C)ccc3-2)c2ccccc2-c2c(-c3ccc4c(c3)-c3cccc5c(-c6cccc7oc8ccccc8c67)ccc(c35)O4)cccc21 ; CC(C)(C)c1ccc2c(c1)C1(c3cc(C(C)(C)C)ccc3-2)c2ccccc2-c2c(-c3ccc4c(c3)-c3cccc5c(-n6c7ccccc7c7ccccc76)ccc(c35)O4)cccc21]

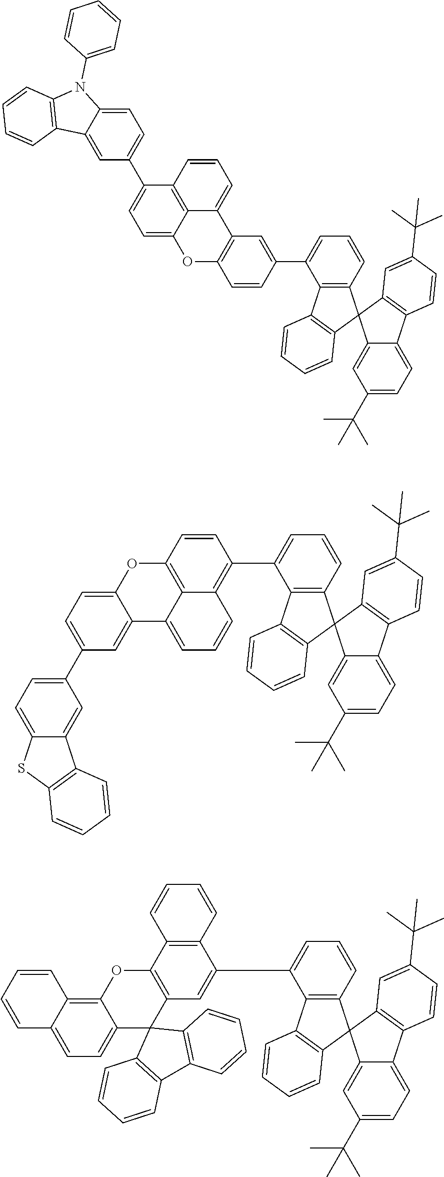 [SMILES: CC(C)(C)c1ccc2c(c1)C1(c3cc(C(C)(C)C)ccc3-2)c2ccccc2-c2c(-c3ccc4c(c3)-c3cccc5c(-c6ccc7c(c6)c6ccccc6n7-c6ccccc6)ccc(c35)O4)cccc21 ; CC(C)(C)c1ccc2c(c1)C1(c3cc(C(C)(C)C)ccc3-2)c2ccccc2-c2c(-c3ccc4c5c(cccc35)-c3cc(-c5ccc6sc7ccccc7c6c5)ccc3O4)cccc21 ; CC(C)(C)c1ccc2c(c1)C1(c3cc(C(C)(C)C)ccc3-2)c2ccccc2-c2c(-c3cc4c(c5ccccc35)Oc3c(ccc5ccccc35)C43c4ccccc4-c4ccccc43)cccc21]

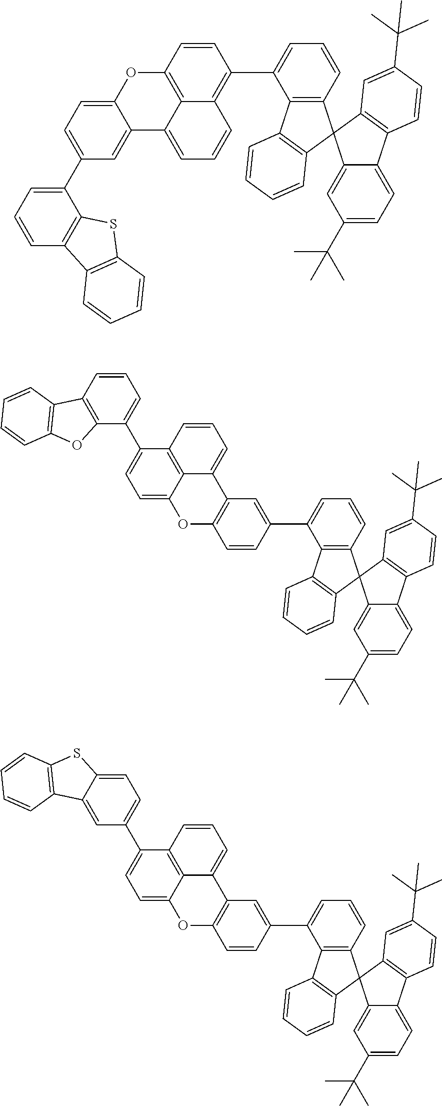 [SMILES: CC(C)(C)c1ccc2c(c1)C1(c3cc(C(C)(C)C)ccc3-2)c2ccccc2-c2c(-c3ccc4c5c(cccc35)-c3cc(-c5cccc6c5sc5ccccc56)ccc3O4)cccc21 ; CC(C)(C)c1ccc2c(c1)C1(c3cc(C(C)(C)C)ccc3-2)c2ccccc2-c2c(-c3ccc4c(c3)-c3cccc5c(-c6cccc7c6oc6ccccc67)ccc(c35)O4)cccc21 ; CC(C)(C)c1ccc2c(c1)C1(c3cc(C(C)(C)C)ccc3-2)c2ccccc2-c2c(-c3ccc4c(c3)-c3cccc5c(-c6ccc7sc8ccccc8c7c6)ccc(c35)O4)cccc21]

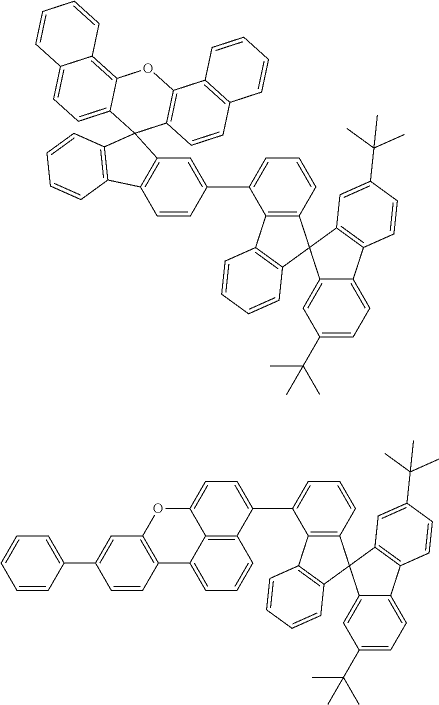 [SMILES: CC(C)(C)c1ccc2c(c1)C1(c3cc(C(C)(C)C)ccc3-2)c2ccccc2-c2c(-c3ccc4c(c3)C3(c5ccccc5-4)c4ccc5ccccc5c4Oc4c3ccc3ccccc43)cccc21 ; CC(C)(C)c1ccc2c(c1)C1(c3cc(C(C)(C)C)ccc3-2)c2ccccc2-c2c(-c3ccc4c5c(cccc35)-c3ccc(-c5ccccc5)cc3O4)cccc21]

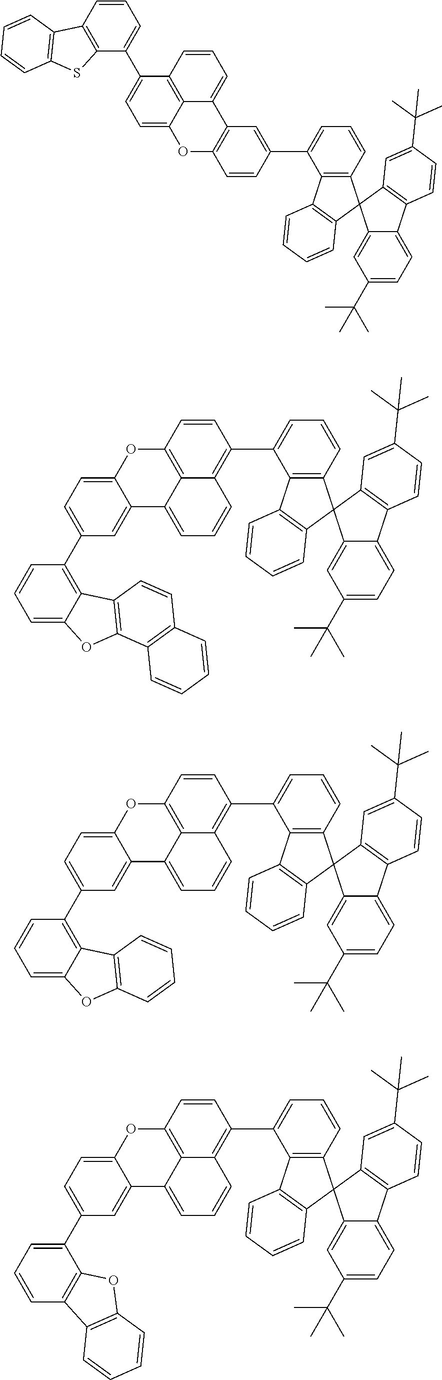 [SMILES: CC(C)(C)c1ccc2c(c1)C1(c3cc(C(C)(C)C)ccc3-2)c2ccccc2-c2c(-c3ccc4c(c3)-c3cccc5c(-c6cccc7c6sc6ccccc67)ccc(c35)O4)cccc21 ; CC(C)(C)c1ccc2c(c1)C1(c3cc(C(C)(C)C)ccc3-2)c2ccccc2-c2c(-c3ccc4c5c(cccc35)-c3cc(-c5cccc6oc7c8ccccc8ccc7c56)ccc3O4)cccc21 ; CC(C)(C)c1ccc2c(c1)C1(c3cc(C(C)(C)C)ccc3-2)c2ccccc2-c2c(-c3ccc4c5c(cccc35)-c3cc(-c5cccc6oc7ccccc7c56)ccc3O4)cccc21 ; CC(C)(C)c1ccc2c(c1)C1(c3cc(C(C)(C)C)ccc3-2)c2ccccc2-c2c(-c3ccc4c5c(cccc35)-c3cc(-c5cccc6c5oc5ccccc56)ccc3O4)cccc21]

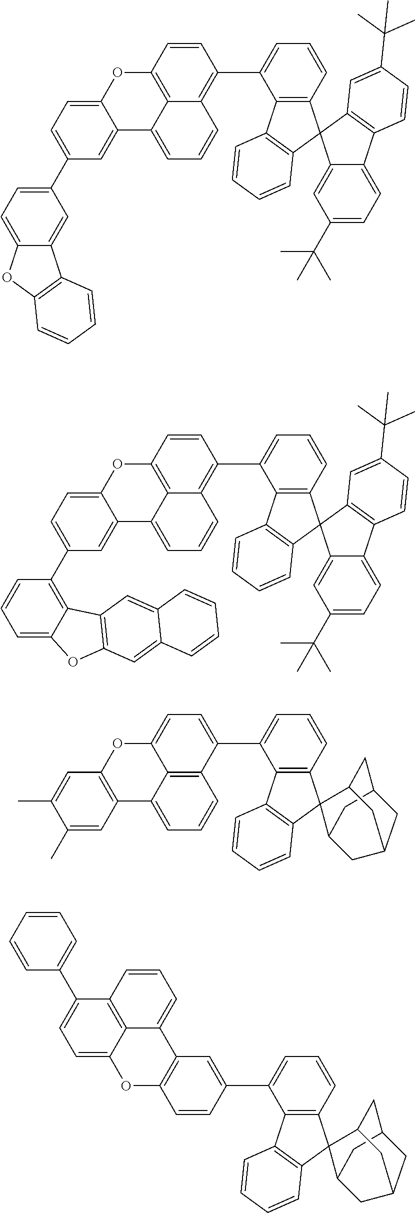

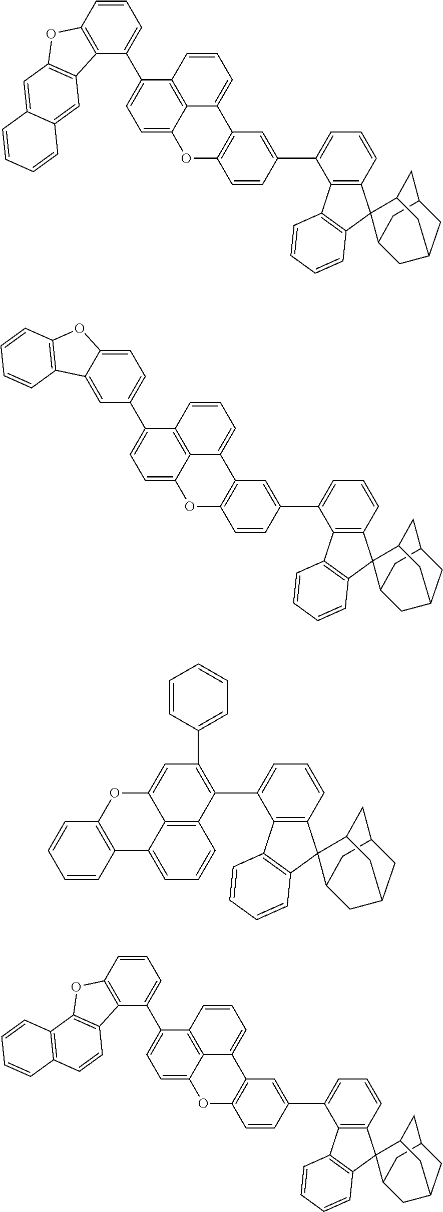 [SMILES: c1ccc2c(c1)-c1c(-c3ccc4c(c3)-c3cccc5c(-c6cccc7oc8cc9ccccc9cc8c67)ccc(c35)O4)cccc1C21C2CC3CC(C2)CC1C3 ; c1ccc2c(c1)-c1c(-c3ccc4c(c3)-c3cccc5c(-c6ccc7oc8ccccc8c7c6)ccc(c35)O4)cccc1C21C2CC3CC(C2)CC1C3 ; c1ccc(-c2cc3c4c(cccc4c2-c2cccc4c2-c2ccccc2C42C4CC5CC(C4)CC2C5)-c2ccccc2O3)cc1 ; c1ccc2c(c1)-c1c(-c3ccc4c(c3)-c3cccc5c(-c6cccc7oc8c9ccccc9ccc8c67)ccc(c35)O4)cccc1C21C2CC3CC(C2)CC1C3]

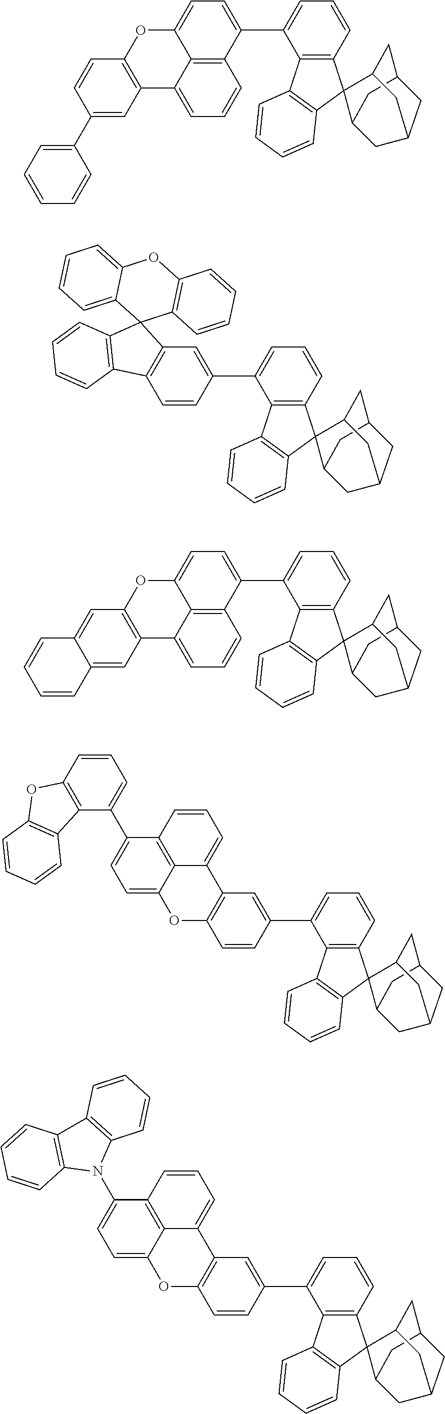 [SMILES: c1ccc(-c2ccc3c(c2)-c2cccc4c(-c5cccc6c5-c5ccccc5C65C6CC7CC(C6)CC5C7)ccc(c24)O3)cc1 ; c1ccc2c(c1)Oc1ccccc1C21c2ccccc2-c2ccc(-c3cccc4c3-c3ccccc3C43C4CC5CC(C4)CC3C5)cc21 ; c1ccc2c(c1)-c1c(-c3ccc4c5c(cccc35)-c3cc5ccccc5cc3O4)cccc1C21C2CC3CC(C2)CC1C3 ; c1ccc2c(c1)-c1c(-c3ccc4c(c3)-c3cccc5c(-c6cccc7oc8ccccc8c67)ccc(c35)O4)cccc1C21C2CC3CC(C2)CC1C3 ; c1ccc2c(c1)-c1c(-c3ccc4c(c3)-c3cccc5c(-n6c7ccccc7c7ccccc76)ccc(c35)O4)cccc1C21C2CC3CC(C2)CC1C3]

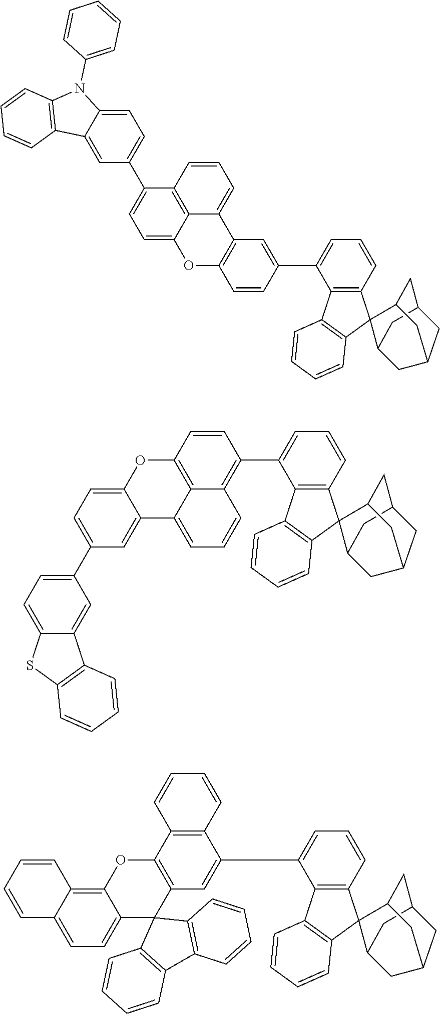 [SMILES: c1ccc(-n2c3ccccc3c3cc(-c4ccc5c6c(cccc46)-c4cc(-c6cccc7c6-c6ccccc6C76C7CC8CC(C7)CC6C8)ccc4O5)ccc32)cc1 ; c1ccc2c(c1)-c1c(-c3ccc4c5c(cccc35)-c3cc(-c5ccc6sc7ccccc7c6c5)ccc3O4)cccc1C21C2CC3CC(C2)CC1C3 ; c1ccc2c(c1)-c1ccccc1C21c2ccc3ccccc3c2Oc2c1cc(-c1cccc3c1-c1ccccc1C31C3CC4CC(C3)CC1C4)c1ccccc21]

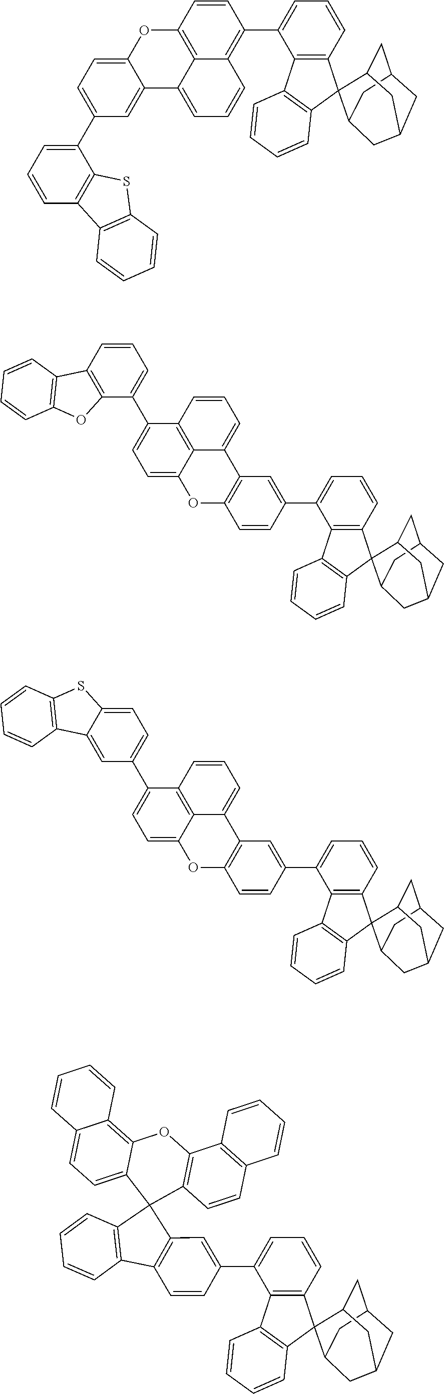 [SMILES: c1ccc2c(c1)-c1c(-c3ccc4c5c(cccc35)-c3cc(-c5cccc6c5sc5ccccc56)ccc3O4)cccc1C21C2CC3CC(C2)CC1C3 ; c1ccc2c(c1)-c1c(-c3ccc4c(c3)-c3cccc5c(-c6cccc7c6oc6ccccc67)ccc(c35)O4)cccc1C21C2CC3CC(C2)CC1C3 ; c1ccc2c(c1)-c1c(-c3ccc4c(c3)-c3cccc5c(-c6ccc7sc8ccccc8c7c6)ccc(c35)O4)cccc1C21C2CC3CC(C2)CC1C3 ; c1ccc2c(c1)-c1ccc(-c3cccc4c3-c3ccccc3C43C4CC5CC(C4)CC3C5)cc1C21c2ccc3ccccc3c2Oc2c1ccc1ccccc21]

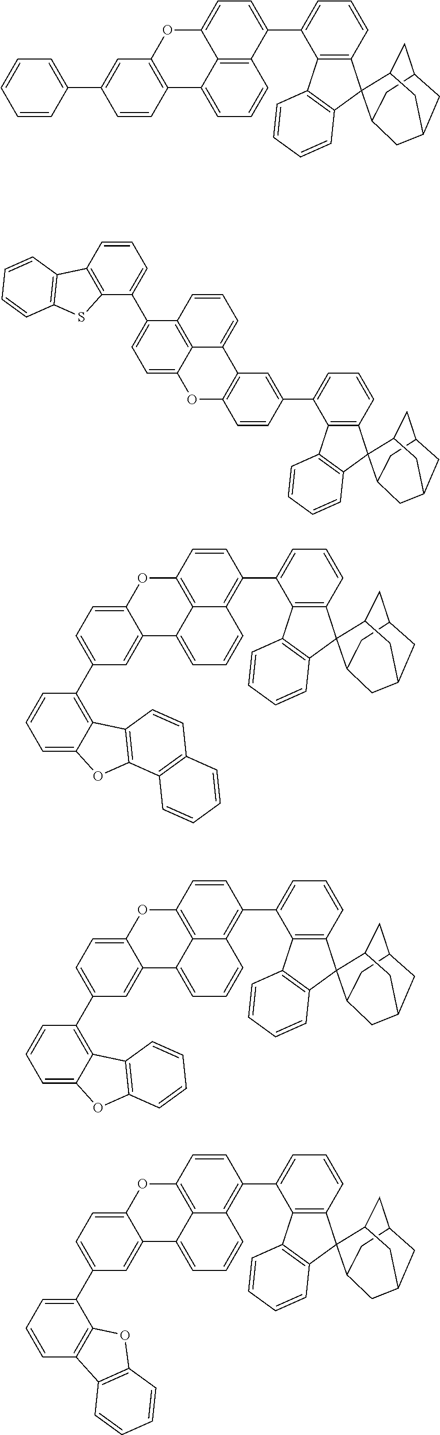

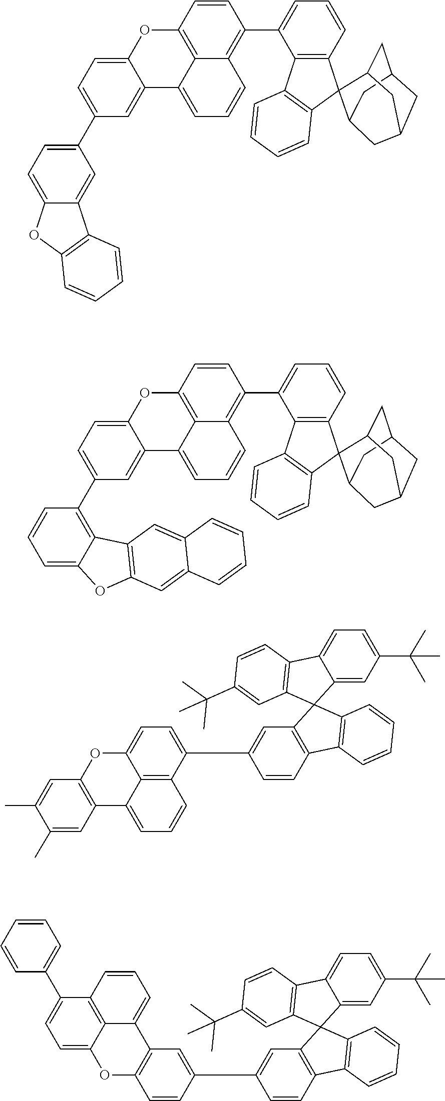 [SMILES: c1ccc2c(c1)-c1c(-c3ccc4c5c(cccc35)-c3cc(-c5ccc6oc7ccccc7c6c5)ccc3O4)cccc1C21C2CC3CC(C2)CC1C3 ; c1ccc2c(c1)-c1c(-c3ccc4c5c(cccc35)-c3cc(-c5cccc6oc7cc8ccccc8cc7c56)ccc3O4)cccc1C21C2CC3CC(C2)CC1C3 ; Cc1cc2c(cc1C)-c1cccc3c(-c4ccc5c(c4)C4(c6ccccc6-5)c5cc(C(C)(C)C)ccc5-c5ccc(C(C)(C)C)cc54)ccc(c13)O2 ; CC(C)(C)c1ccc2c(c1)C1(c3ccccc3-c3ccc(-c4ccc5c(c4)-c4cccc6c(-c7ccccc7)ccc(c46)O5)cc31)c1cc(C(C)(C)C)ccc1-2]

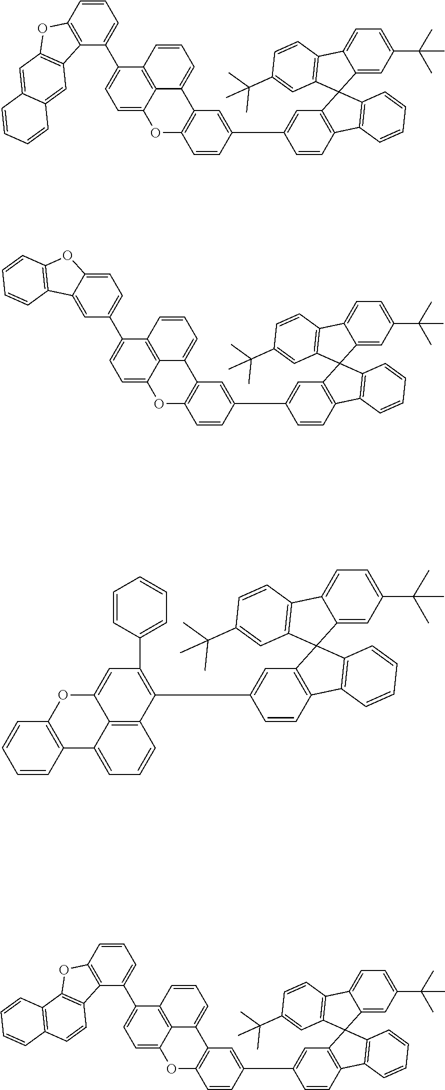 [SMILES: CC(C)(C)c1ccc2c(c1)C1(c3ccccc3-c3ccc(-c4ccc5c(c4)-c4cccc6c(-c7cccc8oc9cc%10ccccc%10cc9c78)ccc(c46)O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7ccc8oc9ccccc9c8c7)ccc(c46)O5)cc31)c1cc(C(C)(C)C)ccc1-2 ; CC(C)(C)c1ccc2c(c1)C1(c3ccccc3-c3ccc(-c4c(-c5ccccc5)cc5c6c(cccc46)-c4ccccc4O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7cccc8oc9c%10ccccc%10ccc9c78)ccc(c46)O5)cc31)c1cc(C(C)(C)C)ccc1-2]

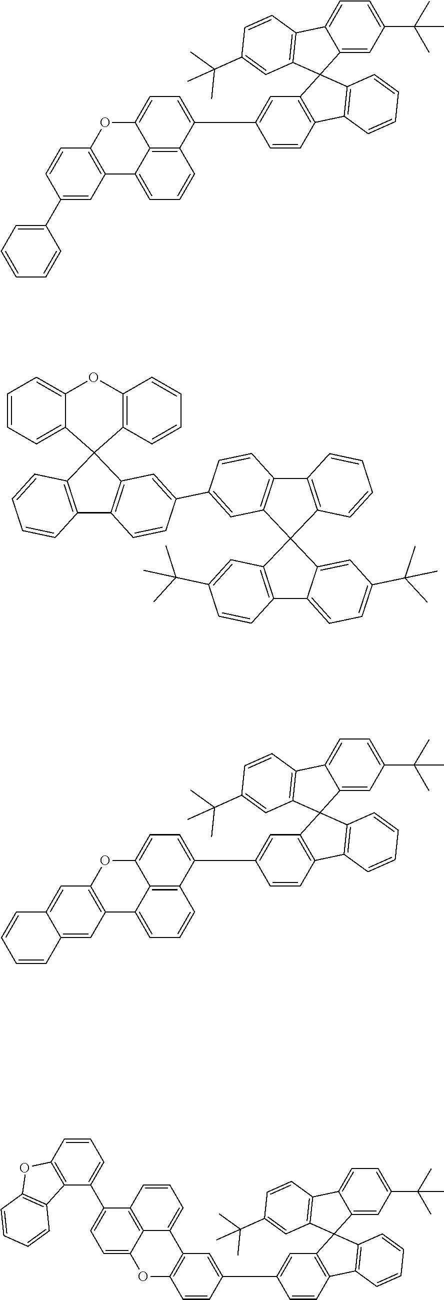 [SMILES: CC(C)(C)c1ccc2c(c1)C1(c3ccccc3-c3ccc(-c4ccc5c6c(cccc46)-c4cc(-c6ccccc6)ccc4O5)cc31)c1cc(C(C)(C)C)ccc1-2 ; CC(C)(C)c1ccc2c(c1)C1(c3ccccc3-c3ccc(-c4ccc5c(c4)C4(c6ccccc6Oc6ccccc64)c4ccccc4-5)cc31)c1cc(C(C)(C)C)ccc1-2 ; CC(C)(C)c1ccc2c(c1)C1(c3ccccc3-c3ccc(-c4ccc5c6c(cccc46)-c4cc6ccccc6cc4O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7cccc8oc9ccccc9c78)ccc(c46)O5)cc31)c1cc(C(C)(C)C)ccc1-2]

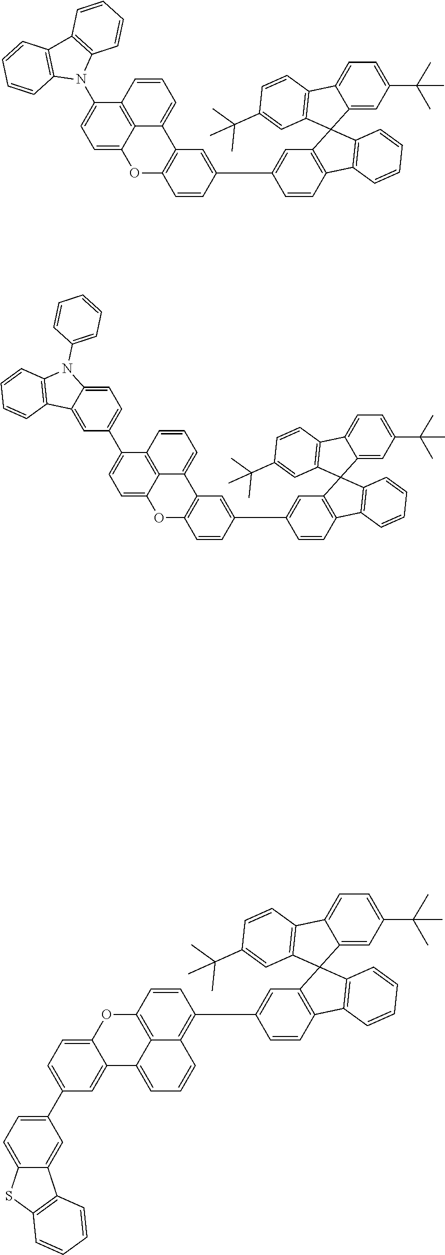 [SMILES: CC(C)(C)c1ccc2c(c1)C1(c3ccccc3-c3ccc(-c4ccc5c(c4)-c4cccc6c(-n7c8ccccc8c8ccccc87)ccc(c46)O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7ccc8c(c7)c7ccccc7n8-c7ccccc7)ccc(c46)O5)cc31)c1cc(C(C)(C)C)ccc1-2 ; CC(C)(C)c1ccc2c(c1)C1(c3ccccc3-c3ccc(-c4ccc5c6c(cccc46)-c4cc(-c6ccc7sc8ccccc8c7c6)ccc4O5)cc31)c1cc(C(C)(C)C)ccc1-2]

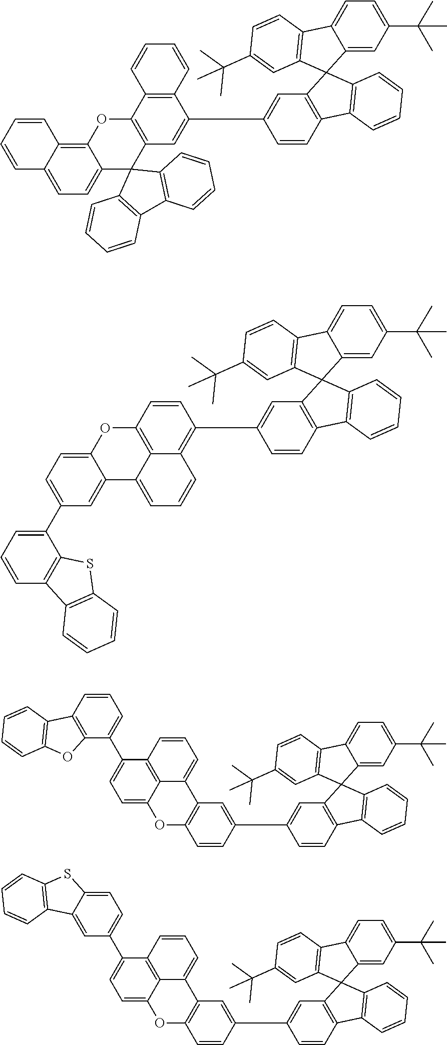 [SMILES: CC(C)(C)c1ccc2c(c1)C1(c3ccccc3-c3ccc(-c4cc5c(c6ccccc46)Oc4c(ccc6ccccc46)C54c5ccccc5-c5ccccc54)cc31)c1cc(C(C)(C)C)ccc1-2 ; CC(C)(C)c1ccc2c(c1)C1(c3ccccc3-c3ccc(-c4ccc5c6c(cccc46)-c4cc(-c6cccc7c6sc6ccccc67)ccc4O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7cccc8c7oc7ccccc78)ccc(c46)O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7ccc8sc9ccccc9c8c7)ccc(c46)O5)cc31)c1cc(C(C)(C)C)ccc1-2]

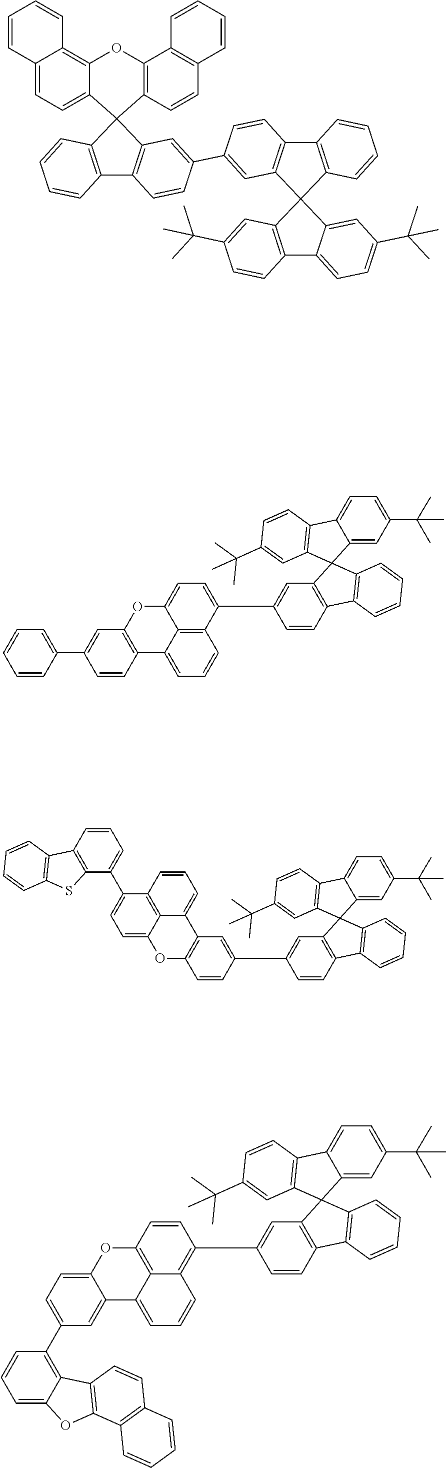 [SMILES: CC(C)(C)c1ccc2c(c1)C1(c3ccccc3-c3ccc(-c4ccc5c(c4)C4(c6ccccc6-5)c5ccc6ccccc6c5Oc5c4ccc4ccccc54)cc31)c1cc(C(C)(C)C)ccc1-2 ; CC(C)(C)c1ccc2c(c1)C1(c3ccccc3-c3ccc(-c4ccc5c6c(cccc46)-c4ccc(-c6ccccc6)cc4O5)cc31)c1cc(C(C)(C)C)ccc1-2 ; CC(C)(C)c1ccc2c(c1)C1(c3ccccc3-c3ccc(-c4ccc5c(c4)-c4cccc6c(-c7cccc8c7sc7ccccc78)ccc(c46)O5)cc31)c1cc(C(C)(C)C)ccc1-2 ; CC(C)(C)c1ccc2c(c1)C1(c3ccccc3-c3ccc(-c4ccc5c6c(cccc46)-c4cc(-c6cccc7oc8c9ccccc9ccc8c67)ccc4O5)cc31)c1cc(C(C)(C)C)ccc1-2]

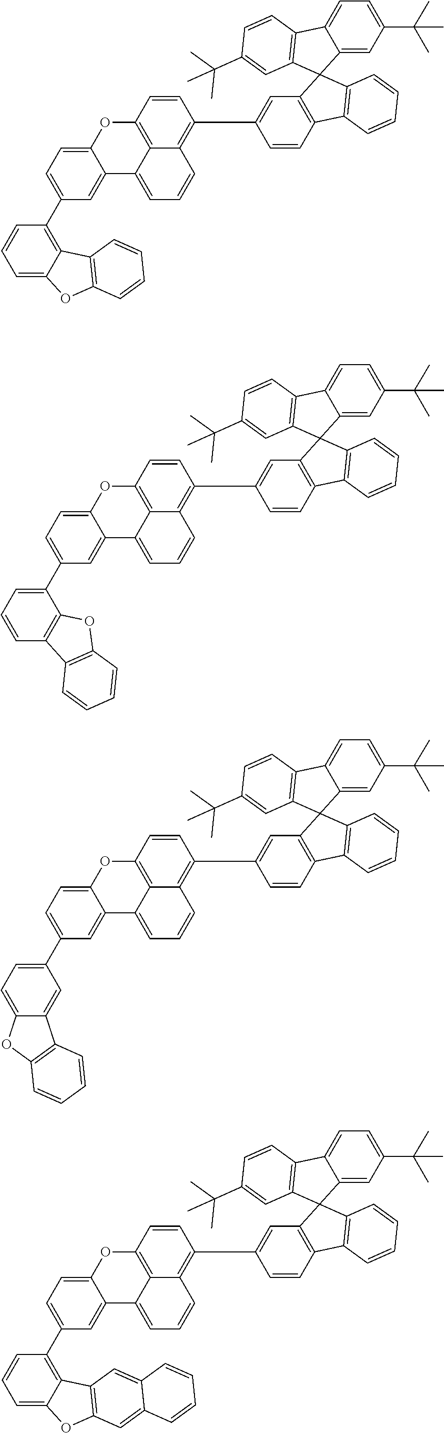 [SMILES: CC(C)(C)c1ccc2c(c1)C1(c3ccccc3-c3ccc(-c4ccc5c6c(cccc46)-c4cc(-c6cccc7oc8ccccc8c67)ccc4O5)cc31)c1cc(C(C)(C)C)ccc1-2 ; CC(C)(C)c1ccc2c(c1)C1(c3ccccc3-c3ccc(-c4ccc5c6c(cccc46)-c4cc(-c6cccc7c6oc6ccccc67)ccc4O5)cc31)c1cc(C(C)(C)C)ccc1-2 ; CC(C)(C)c1ccc2c(c1)C1(c3ccccc3-c3ccc(-c4ccc5c6c(cccc46)-c4cc(-c6ccc7oc8ccccc8c7c6)ccc4O5)cc31)c1cc(C(C)(C)C)ccc1-2 ; CC(C)(C)c1ccc2c(c1)C1(c3ccccc3-c3ccc(-c4ccc5c6c(cccc46)-c4cc(-c6cccc7oc8cc9ccccc9cc8c67)ccc4O5)cc31)c1cc(C(C)(C)C)ccc1-2]

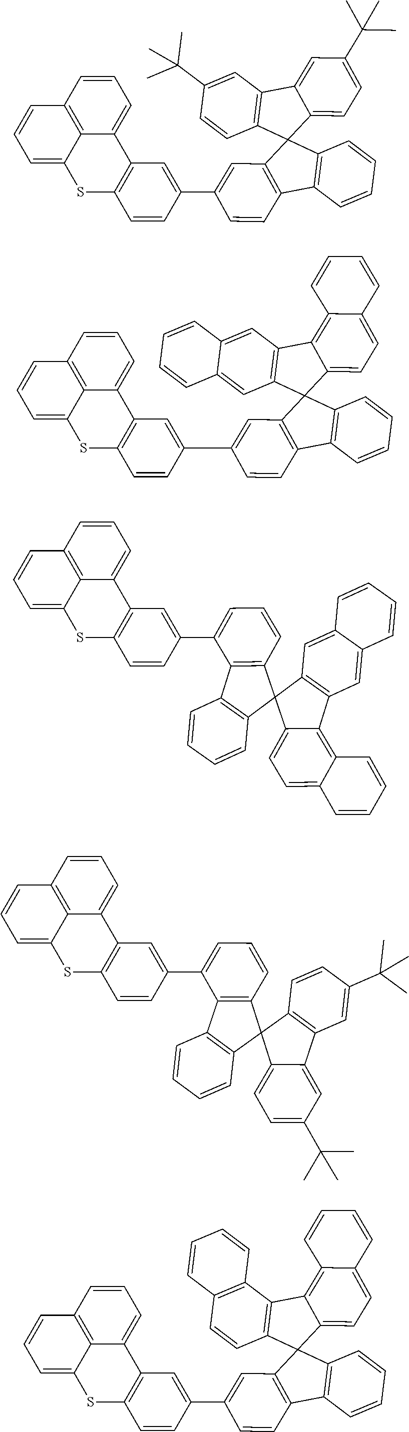

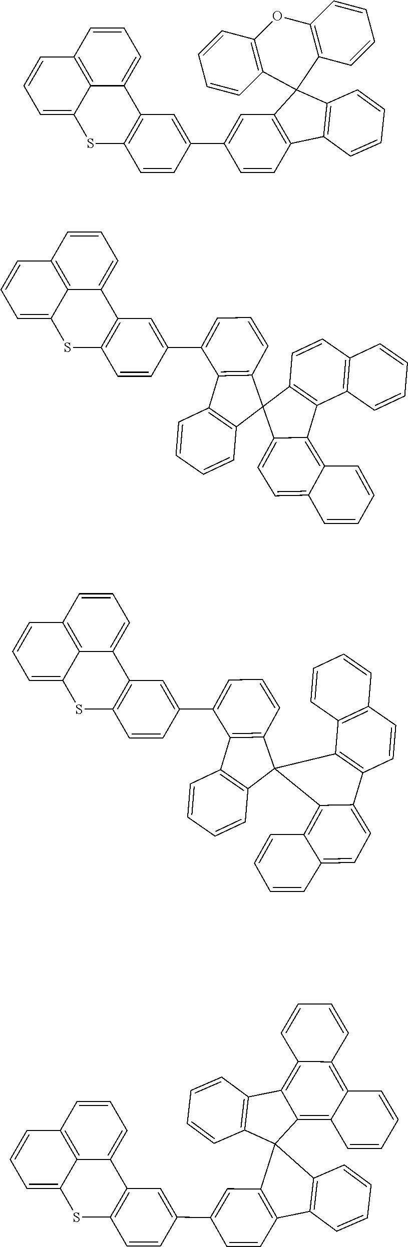 [SMILES: c1ccc2c(c1)Oc1ccccc1C21c2ccccc2-c2ccc(-c3ccc4c(c3)-c3cccc5cccc(c35)S4)cc21 ; c1ccc2c(c1)-c1c(-c3ccc4c(c3)-c3cccc5cccc(c35)S4)cccc1C21c2ccc3ccccc3c2-c2c1ccc1ccccc21 ; c1ccc2c(c1)-c1c(-c3ccc4c(c3)-c3cccc5cccc(c35)S4)cccc1C21c2c(ccc3ccccc23)-c2ccc3ccccc3c21 ; c1ccc2c(c1)-c1ccc(-c3ccc4c(c3)-c3cccc5cccc(c35)S4)cc1C21c2ccccc2-c2c1c1ccccc1c1ccccc21]

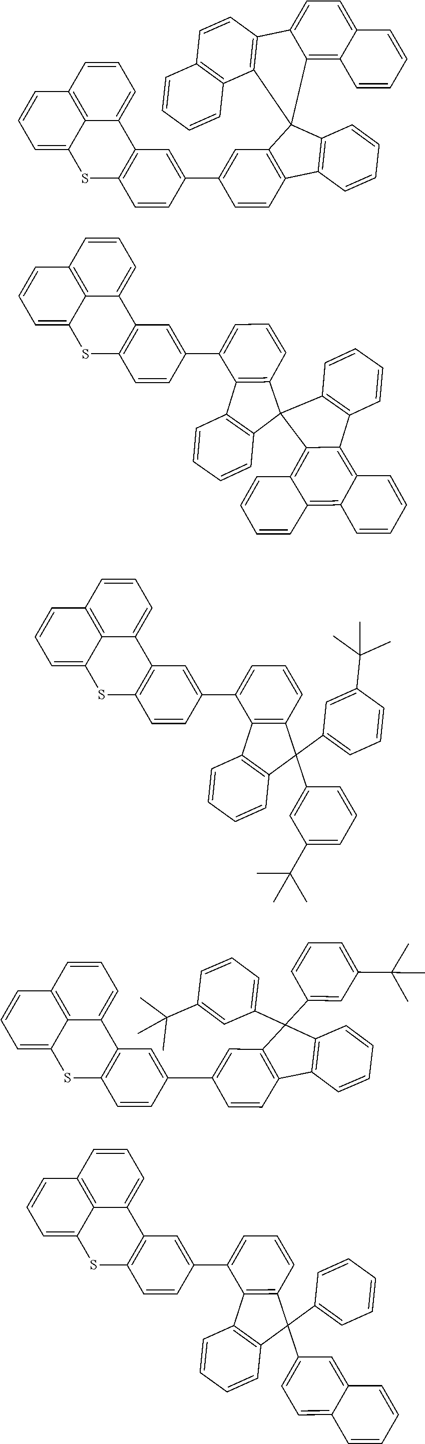

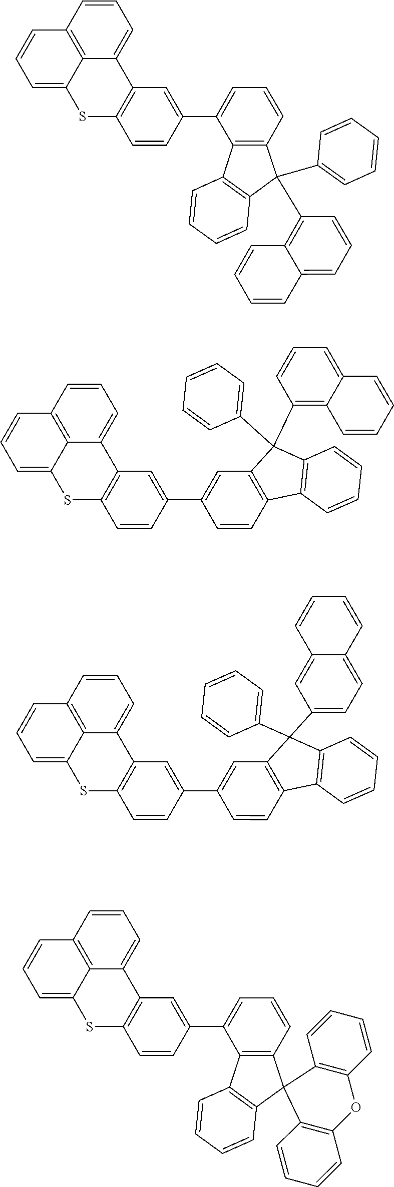

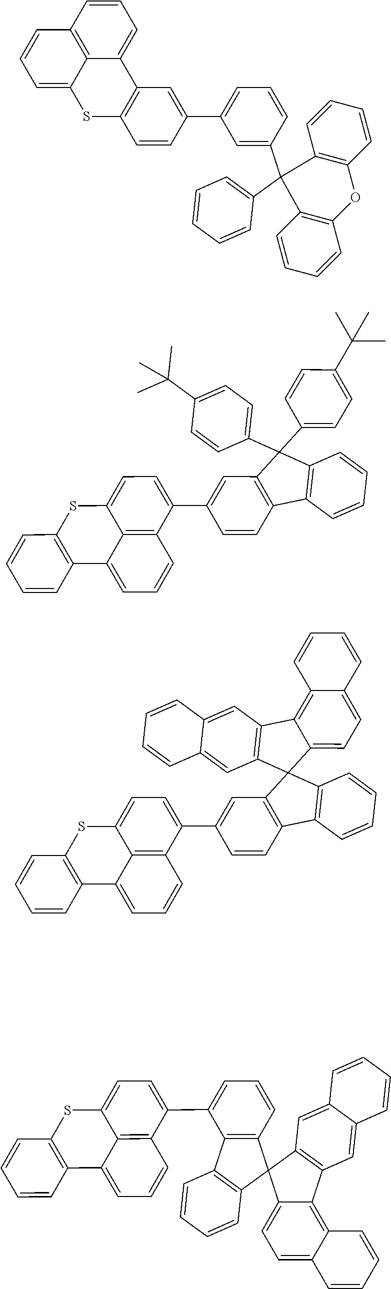

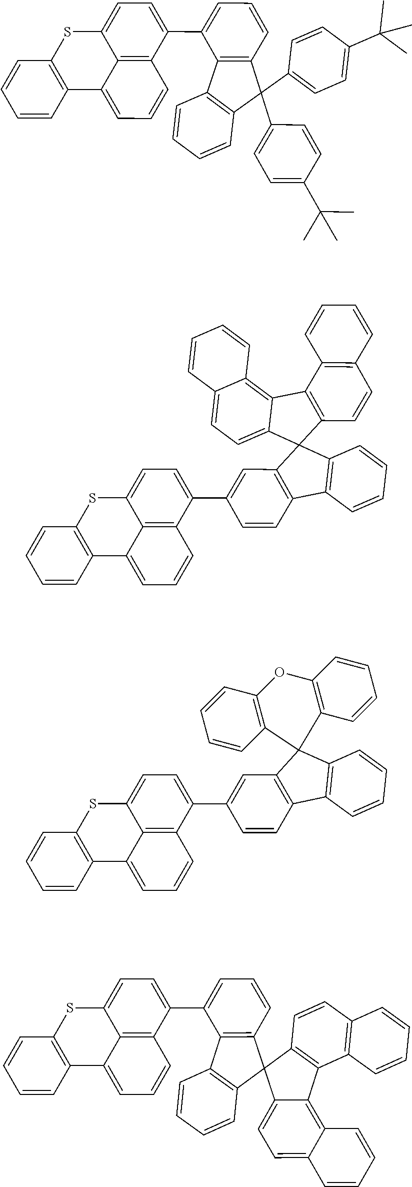 [SMILES: CC(C)(C)c1ccc(C2(c3ccc(C(C)(C)C)cc3)c3ccccc3-c3c(-c4ccc5c6c(cccc46)-c4ccccc4S5)cccc32)cc1 ; c1ccc2c(c1)Sc1ccc(-c3ccc4c(c3)C3(c5ccccc5-4)c4ccc5ccccc5c4-c4c3ccc3ccccc43)c3cccc-2c13 ; c1ccc2c(c1)Oc1ccccc1C21c2ccccc2-c2ccc(-c3ccc4c5c(cccc35)-c3ccccc3S4)cc21 ; c1ccc2c(c1)Sc1ccc(-c3cccc4c3-c3ccccc3C43c4ccc5ccccc5c4-c4c3ccc3ccccc43)c3cccc-2c13]

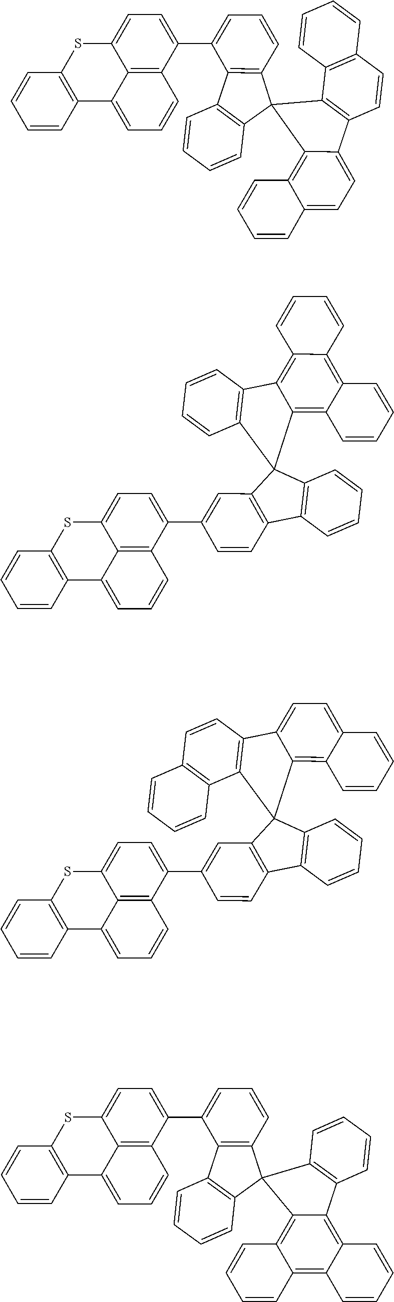 [SMILES: c1ccc2c(c1)Sc1ccc(-c3cccc4c3-c3ccccc3C43c4c(ccc5ccccc45)-c4ccc5ccccc5c43)c3cccc-2c13 ; c1ccc2c(c1)Sc1ccc(-c3ccc4c(c3)C3(c5ccccc5-4)c4ccccc4-c4c3c3ccccc3c3ccccc43)c3cccc-2c13 ; c1ccc2c(c1)Sc1ccc(-c3ccc4c(c3)C3(c5ccccc5-4)c4c(ccc5ccccc45)-c4ccc5ccccc5c43)c3cccc-2c13 ; c1ccc2c(c1)Sc1ccc(-c3cccc4c3-c3ccccc3C43c4ccccc4-c4c3c3ccccc3c3ccccc43)c3cccc-2c13]

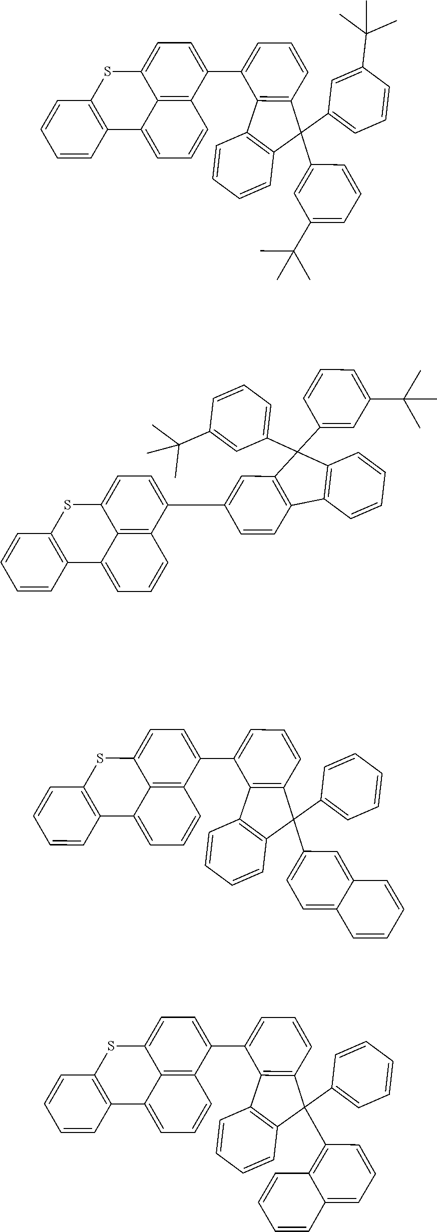 [SMILES: CC(C)(C)c1cccc(C2(c3cccc(C(C)(C)C)c3)c3ccccc3-c3c(-c4ccc5c6c(cccc46)-c4ccccc4S5)cccc32)c1 ; CC(C)(C)c1cccc(C2(c3cccc(C(C)(C)C)c3)c3ccccc3-c3ccc(-c4ccc5c6c(cccc46)-c4ccccc4S5)cc32)c1 ; c1ccc(C2(c3ccc4ccccc4c3)c3ccccc3-c3c(-c4ccc5c6c(cccc46)-c4ccccc4S5)cccc32)cc1 ; c1ccc(C2(c3cccc4ccccc34)c3ccccc3-c3c(-c4ccc5c6c(cccc46)-c4ccccc4S5)cccc32)cc1]

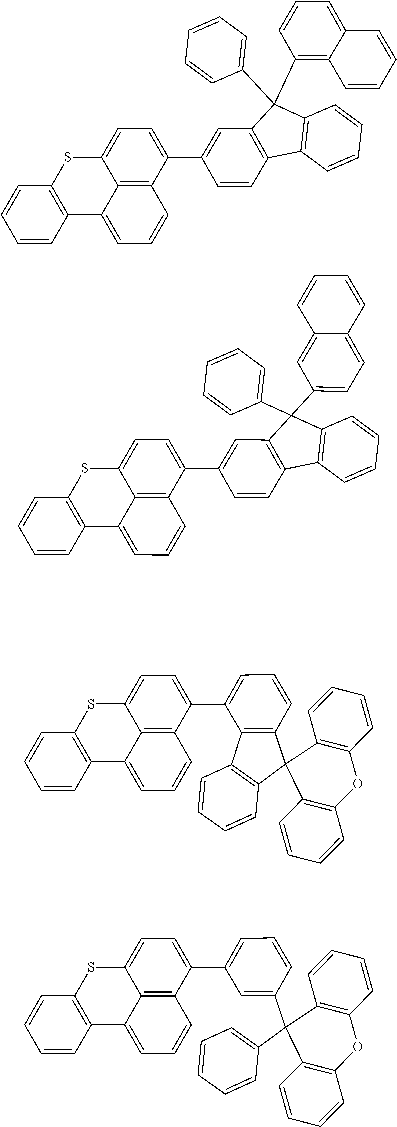 [SMILES: c1ccc(C2(c3cccc4ccccc34)c3ccccc3-c3ccc(-c4ccc5c6c(cccc46)-c4ccccc4S5)cc32)cc1 ; c1ccc(C2(c3ccc4ccccc4c3)c3ccccc3-c3ccc(-c4ccc5c6c(cccc46)-c4ccccc4S5)cc32)cc1 ; c1ccc2c(c1)Oc1ccccc1C21c2ccccc2-c2c(-c3ccc4c5c(cccc35)-c3ccccc3S4)cccc21 ; c1ccc(C2(c3cccc(-c4ccc5c6c(cccc46)-c4ccccc4S5)c3)c3ccccc3Oc3ccccc32)cc1]

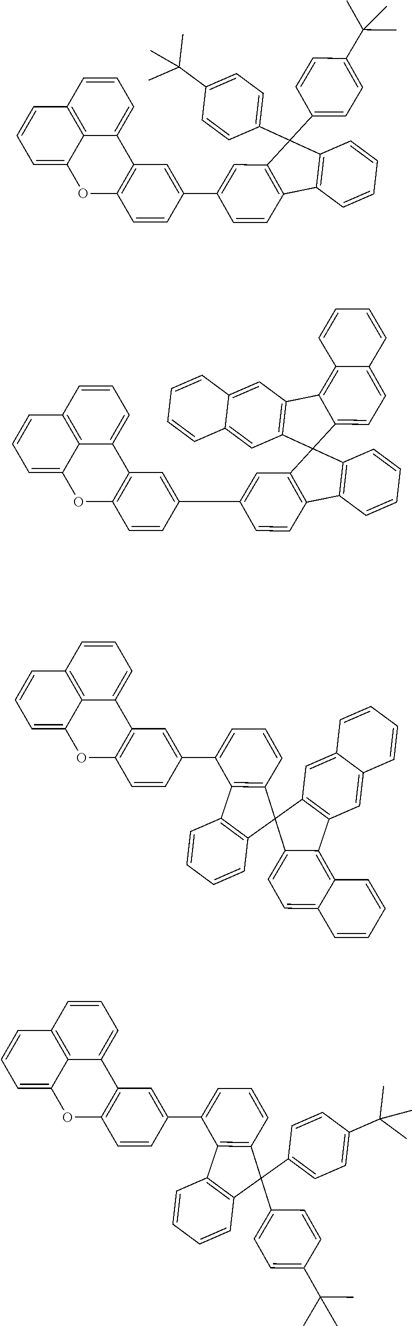 [SMILES: CC(C)(C)c1ccc(C2(c3ccc(C(C)(C)C)cc3)c3ccccc3-c3ccc(-c4ccc5c(c4)-c4cccc6cccc(c46)O5)cc32)cc1 ; c1ccc2c(c1)-c1ccc(-c3ccc4c(c3)-c3cccc5cccc(c35)O4)cc1C21c2cc3ccccc3cc2-c2c1ccc1ccccc21 ; c1ccc2c(c1)-c1c(-c3ccc4c(c3)-c3cccc5cccc(c35)O4)cccc1C21c2cc3ccccc3cc2-c2c1ccc1ccccc21 ; CC(C)(C)c1ccc(C2(c3ccc(C(C)(C)C)cc3)c3ccccc3-c3c(-c4ccc5c(c4)-c4cccc6cccc(c46)O5)cccc32)cc1]

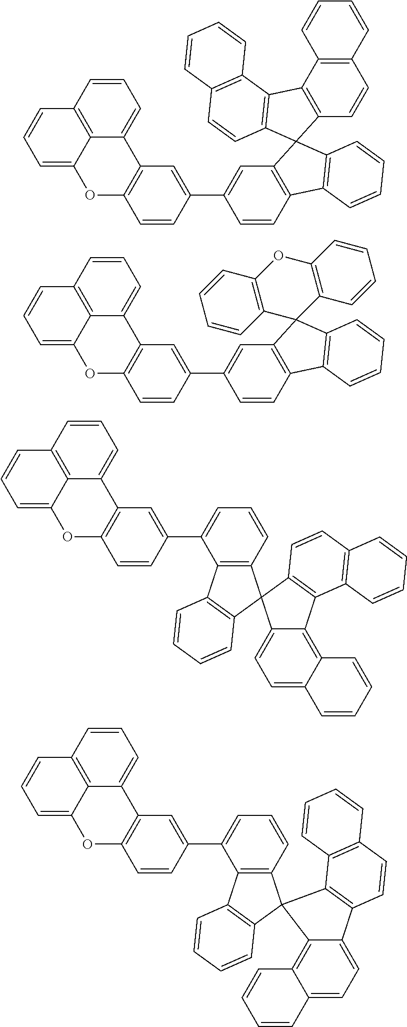

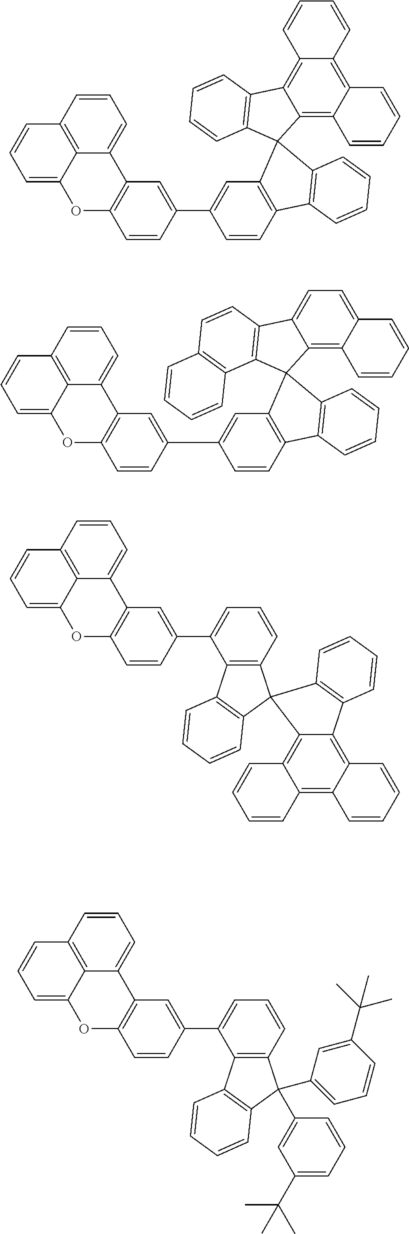 [SMILES: c1ccc2c(c1)-c1ccc(-c3ccc4c(c3)-c3cccc5cccc(c35)O4)cc1C21c2ccccc2-c2c1c1ccccc1c1ccccc21 ; c1ccc2c(c1)-c1ccc(-c3ccc4c(c3)-c3cccc5cccc(c35)O4)cc1C21c2c(ccc3ccccc23)-c2ccc3ccccc3c21 ; c1ccc2c(c1)-c1c(-c3ccc4c(c3)-c3cccc5cccc(c35)O4)cccc1C21c2ccccc2-c2c1c1ccccc1c1ccccc21 ; CC(C)(C)c1cccc(C2(c3cccc(C(C)(C)C)c3)c3ccccc3-c3c(-c4ccc5c(c4)-c4cccc6cccc(c46)O5)cccc32)c1]

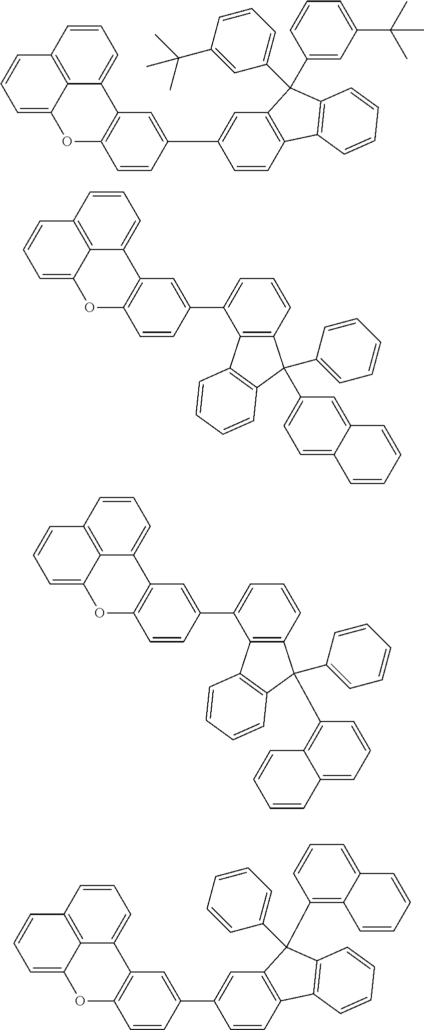

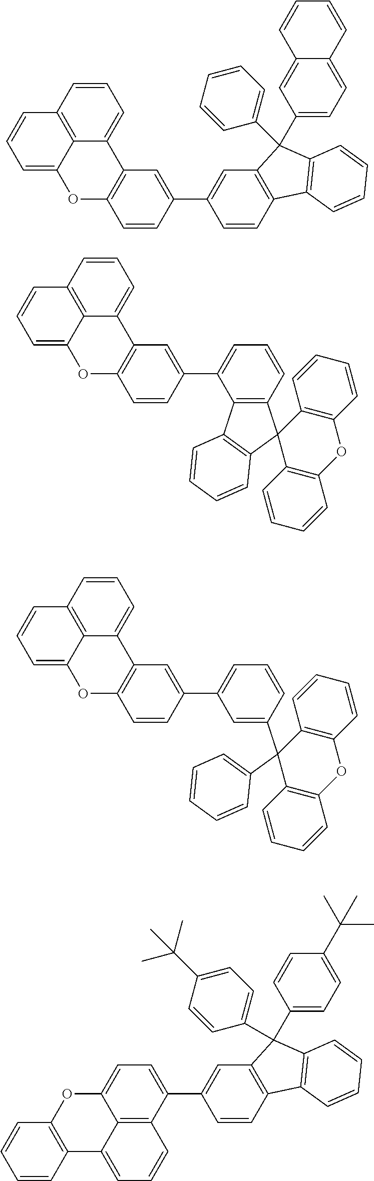

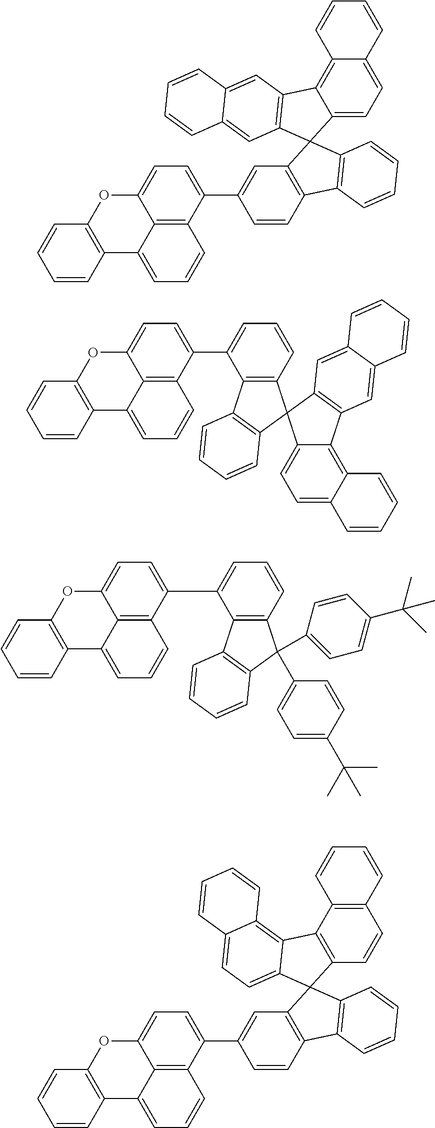

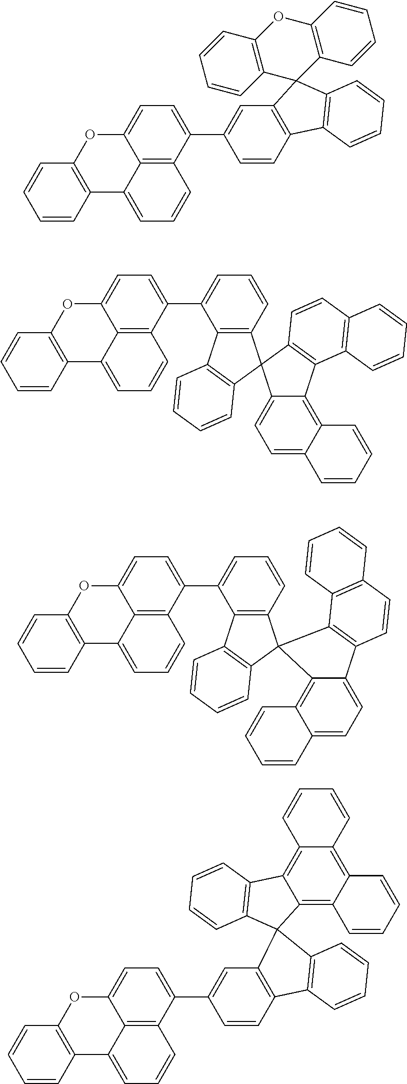

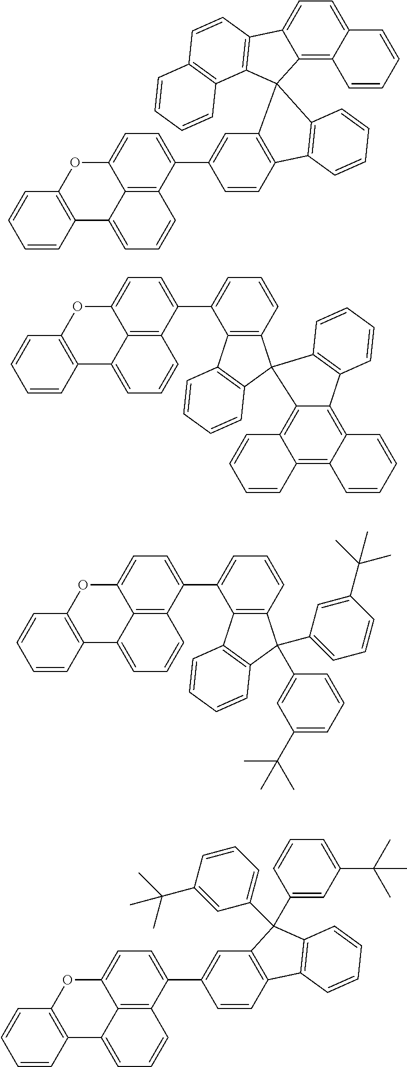

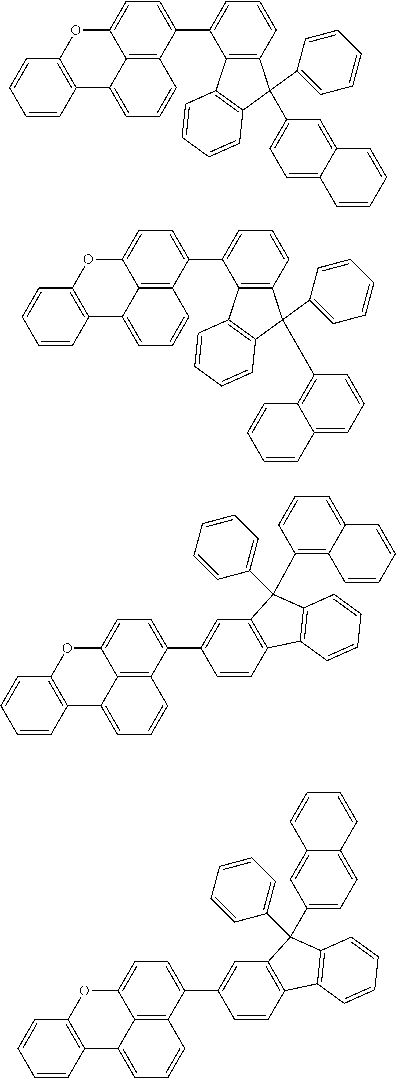

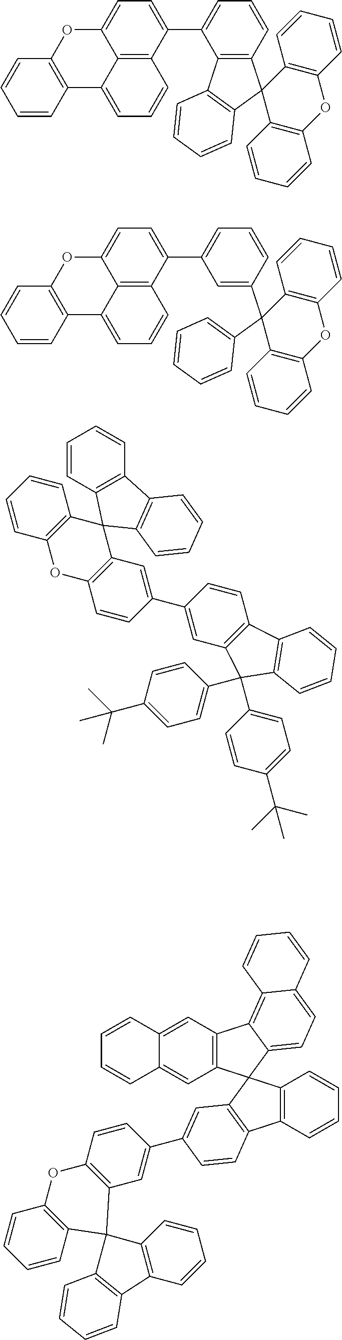 [SMILES: c1ccc2c(c1)Oc1ccc(-c3cccc4c3-c3ccccc3C43c4ccccc4Oc4ccccc43)c3cccc-2c13 ; c1ccc(C2(c3cccc(-c4ccc5c6c(cccc46)-c4ccccc4O5)c3)c3ccccc3Oc3ccccc32)cc1 ; CC(C)(C)c1ccc(C2(c3ccc(C(C)(C)C)cc3)c3ccccc3-c3ccc(-c4ccc5c(c4)C4(c6ccccc6O5)c5ccccc5-c5ccccc54)cc32)cc1 ; c1ccc2c(c1)Oc1ccc(-c3ccc4c(c3)C3(c5ccccc5-4)c4cc5ccccc5cc4-c4c3ccc3ccccc43)cc1C21c2ccccc2-c2ccccc21]

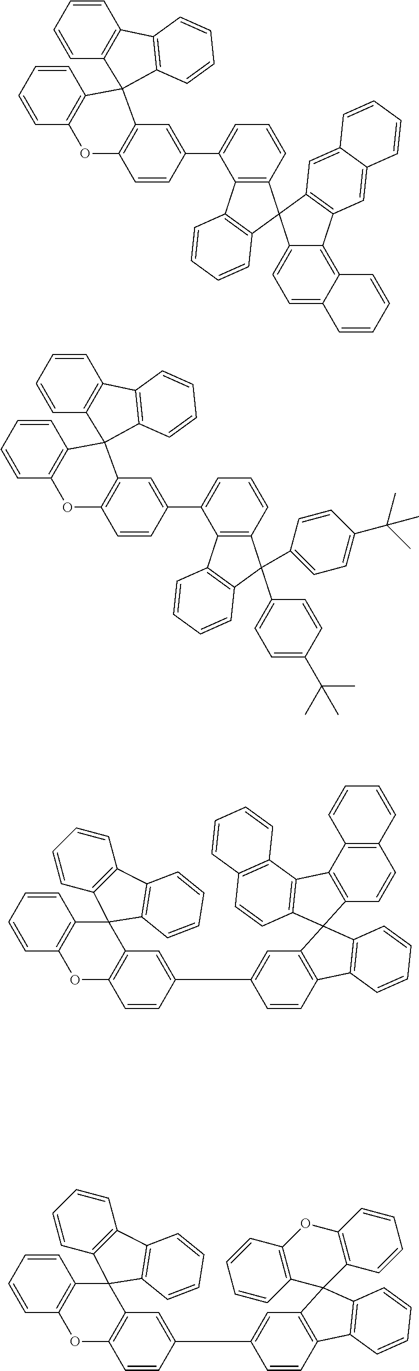 [SMILES: c1ccc2c(c1)Oc1ccc(-c3cccc4c3-c3ccccc3C43c4cc5ccccc5cc4-c4c3ccc3ccccc43)cc1C21c2ccccc2-c2ccccc21 ; CC(C)(C)c1ccc(C2(c3ccc(C(C)(C)C)cc3)c3ccccc3-c3c(-c4ccc5c(c4)C4(c6ccccc6O5)c5ccccc5-c5ccccc54)cccc32)cc1 ; c1ccc2c(c1)Oc1ccc(-c3ccc4c(c3)C3(c5ccccc5-4)c4ccc5ccccc5c4-c4c3ccc3ccccc43)cc1C21c2ccccc2-c2ccccc21 ; c1ccc2c(c1)Oc1ccc(-c3ccc4c(c3)C3(c5ccccc5Oc5ccccc53)c3ccccc3-4)cc1C21c2ccccc2-c2ccccc21]

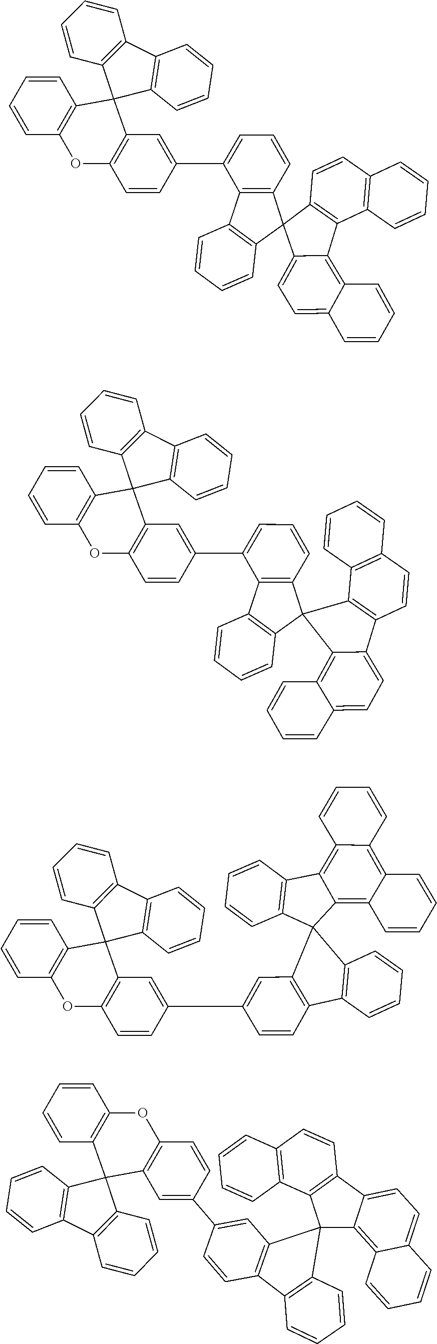 [SMILES: c1ccc2c(c1)Oc1ccc(-c3cccc4c3-c3ccccc3C43c4ccc5ccccc5c4-c4c3ccc3ccccc43)cc1C21c2ccccc2-c2ccccc21 ; c1ccc2c(c1)Oc1ccc(-c3cccc4c3-c3ccccc3C43c4c(ccc5ccccc45)-c4ccc5ccccc5c43)cc1C21c2ccccc2-c2ccccc21 ; c1ccc2c(c1)Oc1ccc(-c3ccc4c(c3)C3(c5ccccc5-4)c4ccccc4-c4c3c3ccccc3c3ccccc43)cc1C21c2ccccc2-c2ccccc21 ; c1ccc2c(c1)Oc1ccc(-c3ccc4c(c3)C3(c5ccccc5-4)c4c(ccc5ccccc45)-c4ccc5ccccc5c43)cc1C21c2ccccc2-c2ccccc21]

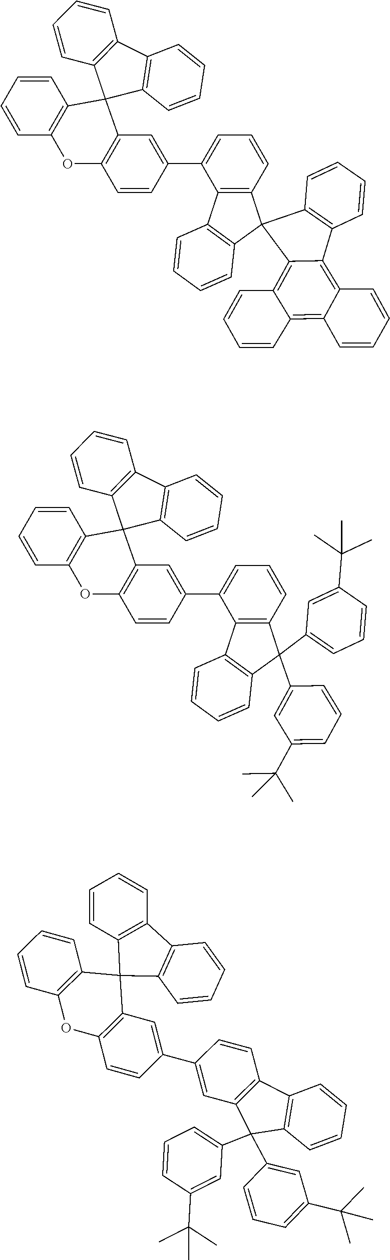 [SMILES: c1ccc2c(c1)Oc1ccc(-c3cccc4c3-c3ccccc3C43c4ccccc4-c4c3c3ccccc3c3ccccc43)cc1C21c2ccccc2-c2ccccc21 ; CC(C)(C)c1cccc(C2(c3cccc(C(C)(C)C)c3)c3ccccc3-c3c(-c4ccc5c(c4)C4(c6ccccc6O5)c5ccccc5-c5ccccc54)cccc32)c1 ; CC(C)(C)c1cccc(C2(c3cccc(C(C)(C)C)c3)c3ccccc3-c3ccc(-c4ccc5c(c4)C4(c6ccccc6O5)c5ccccc5-c5ccccc54)cc32)c1]

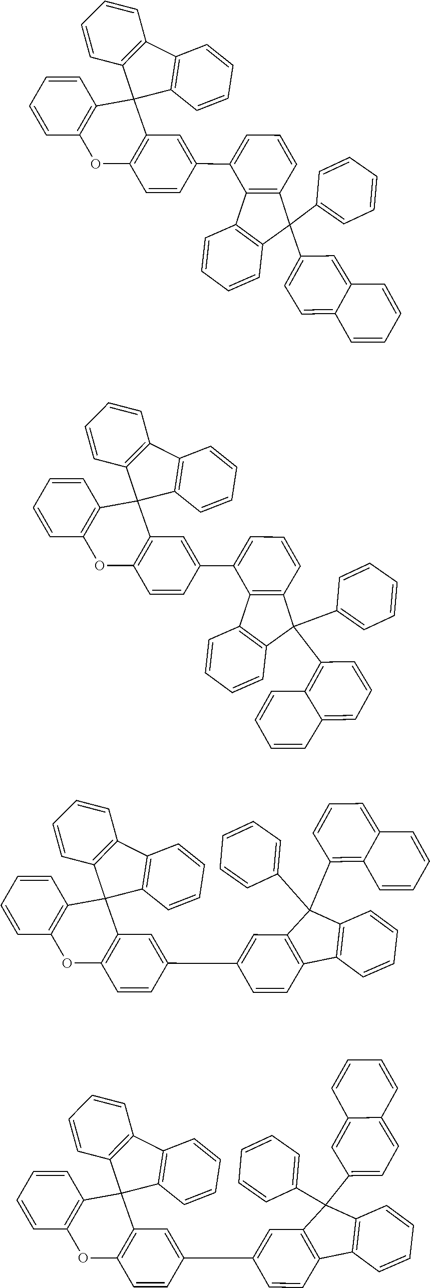 [SMILES: c1ccc(C2(c3ccc4ccccc4c3)c3ccccc3-c3c(-c4ccc5c(c4)C4(c6ccccc6O5)c5ccccc5-c5ccccc54)cccc32)cc1 ; c1ccc(C2(c3cccc4ccccc34)c3ccccc3-c3c(-c4ccc5c(c4)C4(c6ccccc6O5)c5ccccc5-c5ccccc54)cccc32)cc1 ; c1ccc(C2(c3cccc4ccccc34)c3ccccc3-c3ccc(-c4ccc5c(c4)C4(c6ccccc6O5)c5ccccc5-c5ccccc54)cc32)cc1 ; c1ccc(C2(c3ccc4ccccc4c3)c3ccccc3-c3ccc(-c4ccc5c(c4)C4(c6ccccc6O5)c5ccccc5-c5ccccc54)cc32)cc1]

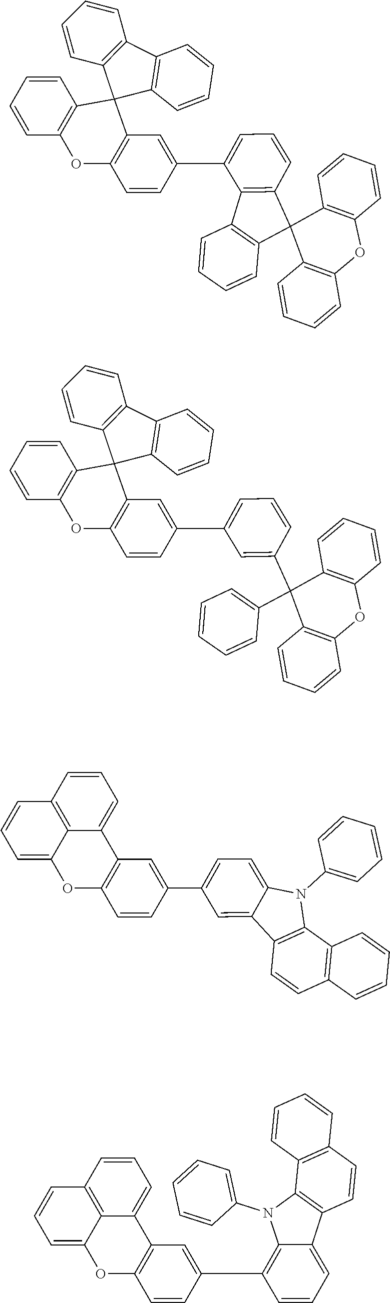 [SMILES: c1ccc2c(c1)Oc1ccc(-c3cccc4c3-c3ccccc3C43c4ccccc4Oc4ccccc43)cc1C21c2ccccc2-c2ccccc21 ; c1ccc(C2(c3cccc(-c4ccc5c(c4)C4(c6ccccc6O5)c5ccccc5-c5ccccc54)c3)c3ccccc3Oc3ccccc32)cc1 ; c1ccc(-n2c3ccc(-c4ccc5c(c4)-c4cccc6cccc(c46)O5)cc3c3ccc4ccccc4c32)cc1 ; c1ccc(-n2c3c(-c4ccc5c(c4)-c4cccc6cccc(c46)O5)cccc3c3ccc4ccccc4c32)cc1]

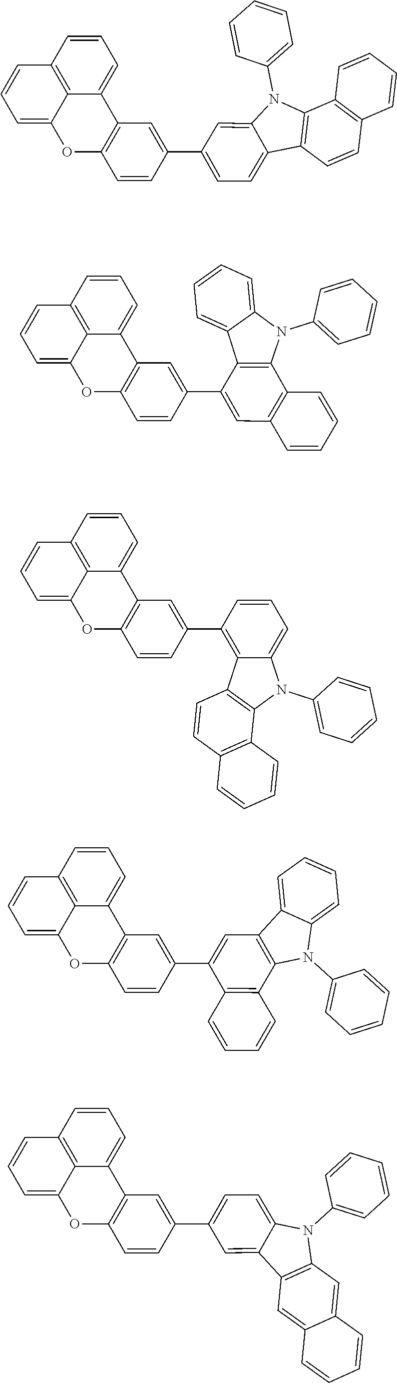 [SMILES: c1ccc(-n2c3cc(-c4ccc5c(c4)-c4cccc6cccc(c46)O5)ccc3c3ccc4ccccc4c32)cc1 ; c1ccc(-n2c3ccccc3c3c(-c4ccc5c(c4)-c4cccc6cccc(c46)O5)cc4ccccc4c32)cc1 ; c1ccc(-n2c3cccc(-c4ccc5c(c4)-c4cccc6cccc(c46)O5)c3c3ccc4ccccc4c32)cc1 ; c1ccc(-n2c3ccccc3c3cc(-c4ccc5c(c4)-c4cccc6cccc(c46)O5)c4ccccc4c32)cc1 ; c1ccc(-n2c3ccc(-c4ccc5c(c4)-c4cccc6cccc(c46)O5)cc3c3cc4ccccc4cc32)cc1]

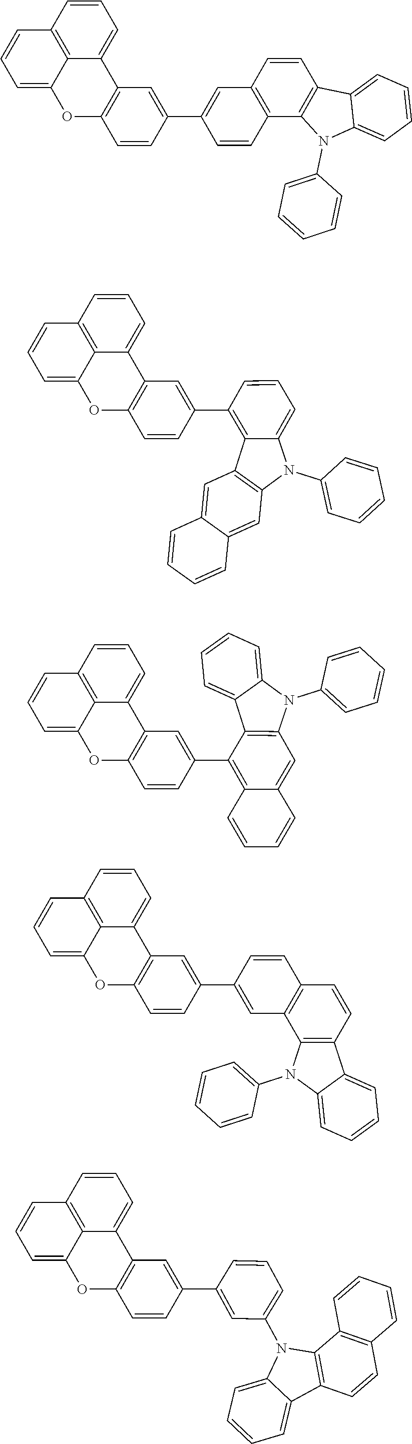 [SMILES: c1ccc(-n2c3ccccc3c3ccc4cc(-c5ccc6c(c5)-c5cccc7cccc(c57)O6)ccc4c32)cc1 ; c1ccc(-n2c3cc4ccccc4cc3c3c(-c4ccc5c(c4)-c4cccc6cccc(c46)O5)cccc32)cc1 ; c1ccc(-n2c3ccccc3c3c(-c4ccc5c(c4)-c4cccc6cccc(c46)O5)c4ccccc4cc32)cc1 ; c1ccc(-n2c3ccccc3c3ccc4ccc(-c5ccc6c(c5)-c5cccc7cccc(c57)O6)cc4c32)cc1 ; c1cc(-c2ccc3c(c2)-c2cccc4cccc(c24)O3)cc(-n2c3ccccc3c3ccc4ccccc4c32)c1]

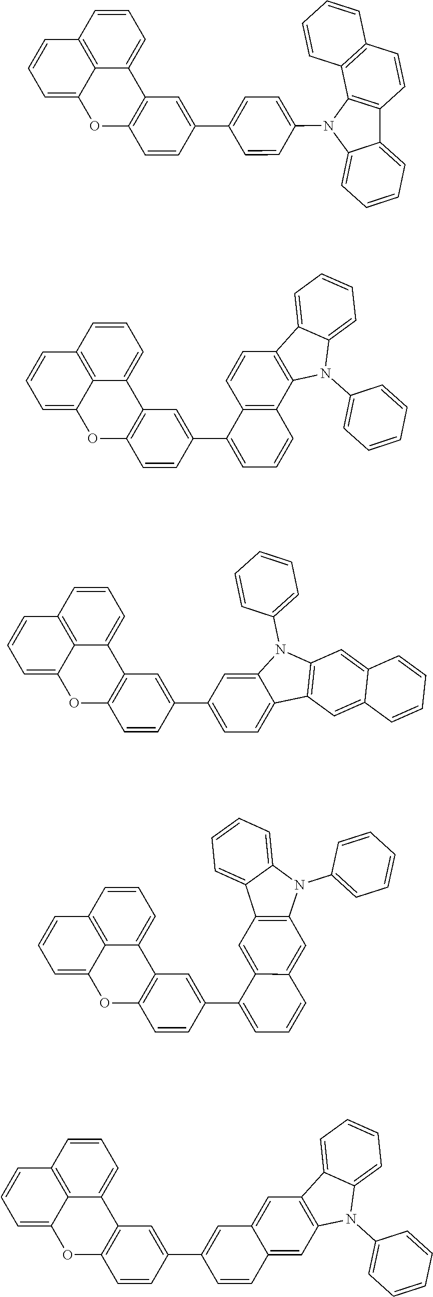 [SMILES: c1cc2c3c(cccc3c1)-c1cc(-c3ccc(-n4c5ccccc5c5ccc6ccccc6c54)cc3)ccc1O2 ; c1ccc(-n2c3ccccc3c3ccc4c(-c5ccc6c(c5)-c5cccc7cccc(c57)O6)cccc4c32)cc1 ; c1ccc(-n2c3cc(-c4ccc5c(c4)-c4cccc6cccc(c46)O5)ccc3c3cc4ccccc4cc32)cc1 ; c1ccc(-n2c3ccccc3c3cc4c(-c5ccc6c(c5)-c5cccc7cccc(c57)O6)cccc4cc32)cc1 ; c1ccc(-n2c3ccccc3c3cc4cc(-c5ccc6c(c5)-c5cccc7cccc(c57)O6)ccc4cc32)cc1]

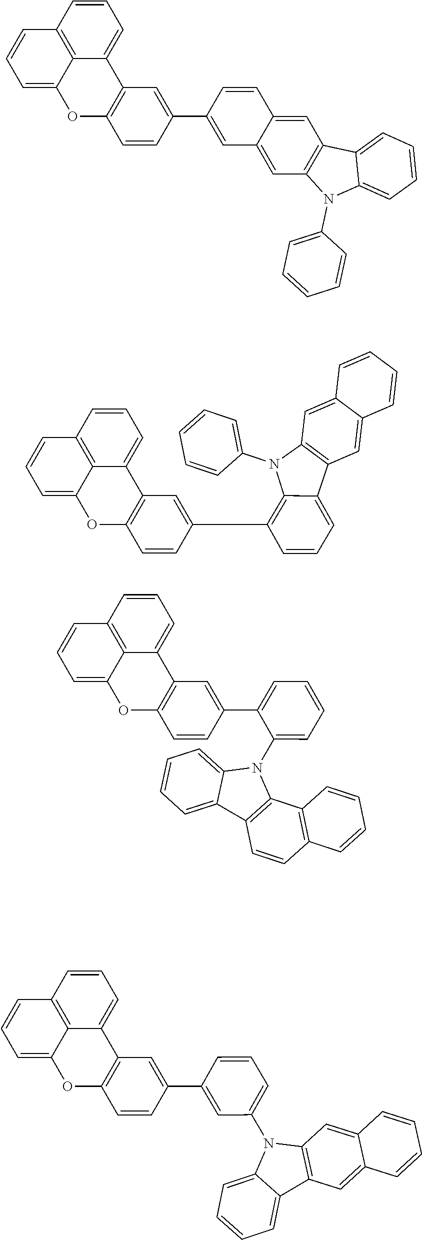

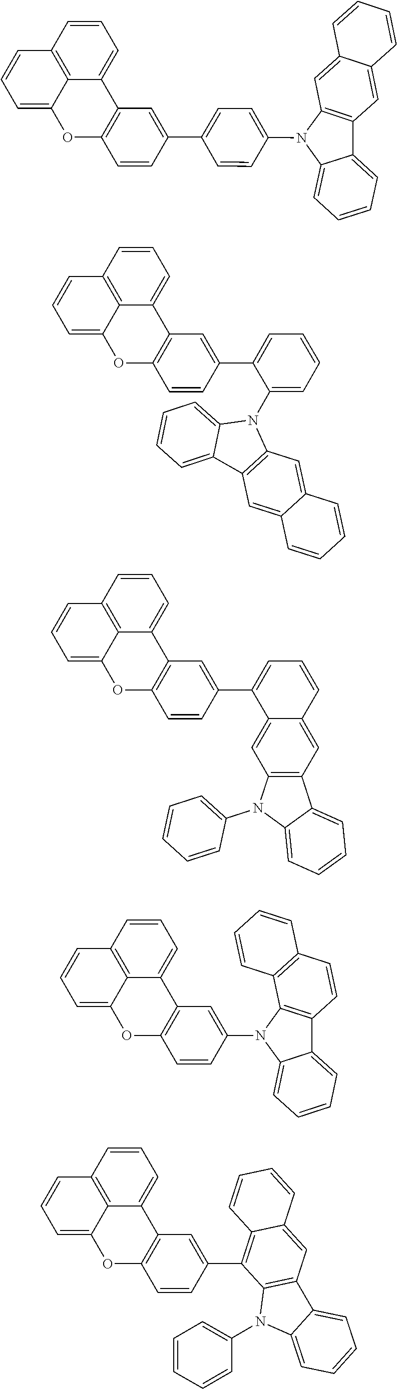 [SMILES: c1ccc2cc3c(cc2c1)c1ccccc1n3-c1ccc(-c2ccc3c(c2)-c2cccc4cccc(c24)O3)cc1 ; c1ccc(-n2c3ccccc3c3cc4ccccc4cc32)c(-c2ccc3c(c2)-c2cccc4cccc(c24)O3)c1 ; c1ccc(-n2c3ccccc3c3cc4cccc(-c5ccc6c(c5)-c5cccc7cccc(c57)O6)c4cc32)cc1 ; c1cc2c3c(cccc3c1)-c1cc(-n3c4ccccc4c4ccc5ccccc5c43)ccc1O2 ; c1ccc(-n2c3ccccc3c3cc4ccccc4c(-c4ccc5c(c4)-c4cccc6cccc(c46)O5)c32)cc1]

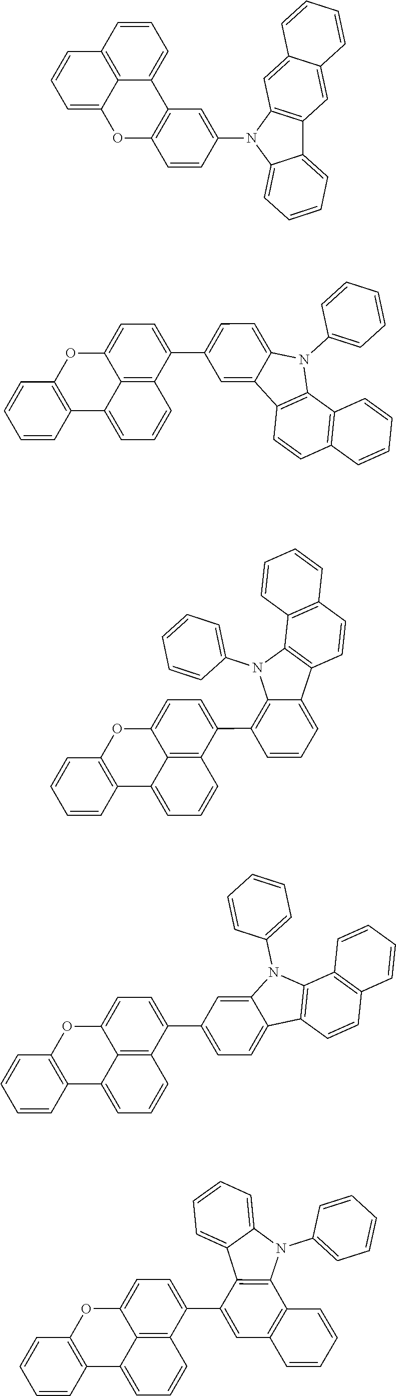 [SMILES: c1ccc2cc3c(cc2c1)c1ccccc1n3-c1ccc2c(c1)-c1cccc3cccc(c13)O2 ; c1ccc(-n2c3ccc(-c4ccc5c6c(cccc46)-c4ccccc4O5)cc3c3ccc4ccccc4c32)cc1 ; c1ccc(-n2c3c(-c4ccc5c6c(cccc46)-c4ccccc4O5)cccc3c3ccc4ccccc4c32)cc1 ; c1ccc(-n2c3cc(-c4ccc5c6c(cccc46)-c4ccccc4O5)ccc3c3ccc4ccccc4c32)cc1 ; c1ccc(-n2c3ccccc3c3c(-c4ccc5c6c(cccc46)-c4ccccc4O5)cc4ccccc4c32)cc1]

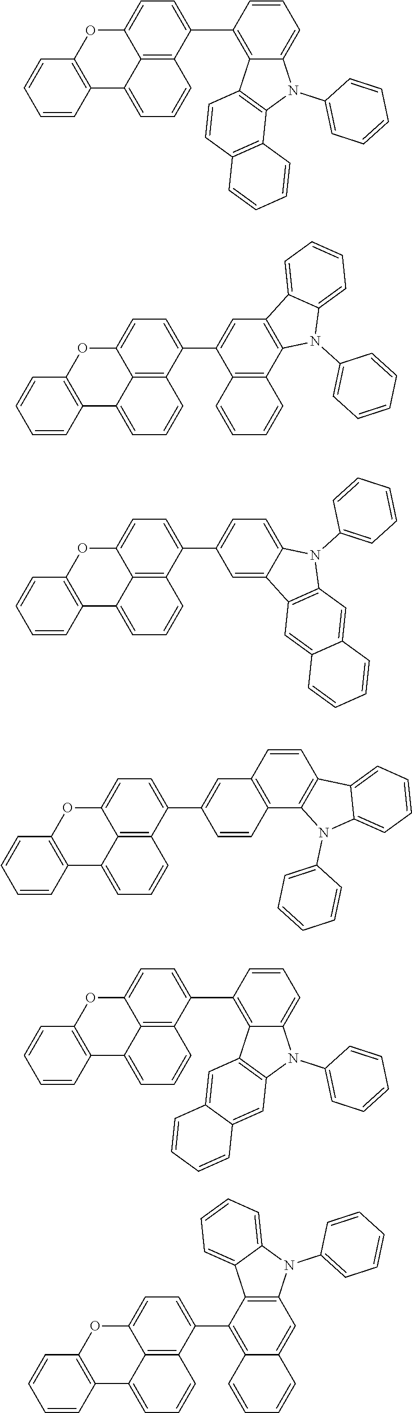

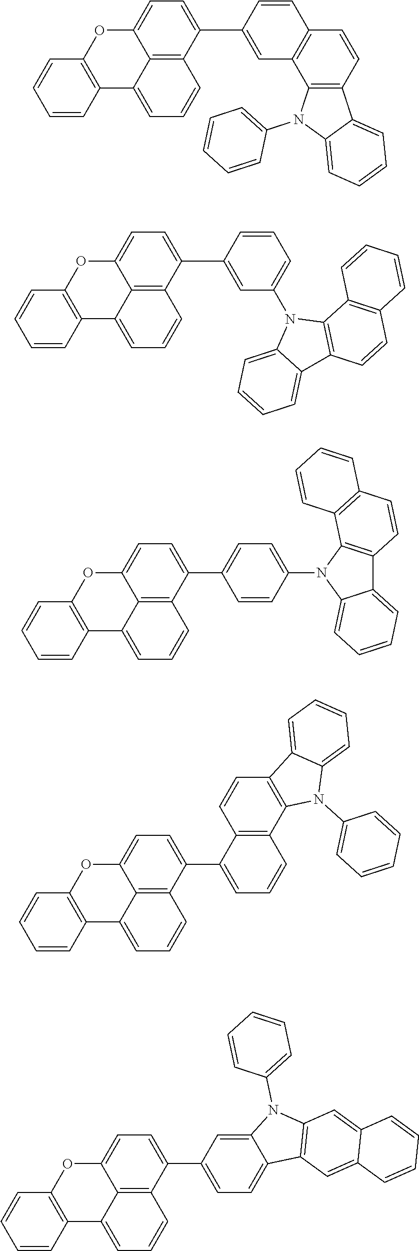 [SMILES: c1ccc(-n2c3ccccc3c3ccc4ccc(-c5ccc6c7c(cccc57)-c5ccccc5O6)cc4c32)cc1 ; c1cc(-c2ccc3c4c(cccc24)-c2ccccc2O3)cc(-n2c3ccccc3c3ccc4ccccc4c32)c1 ; c1ccc2c(c1)Oc1ccc(-c3ccc(-n4c5ccccc5c5ccc6ccccc6c54)cc3)c3cccc-2c13 ; c1ccc(-n2c3ccccc3c3ccc4c(-c5ccc6c7c(cccc57)-c5ccccc5O6)cccc4c32)cc1 ; c1ccc(-n2c3cc(-c4ccc5c6c(cccc46)-c4ccccc4O5)ccc3c3cc4ccccc4cc32)cc1]

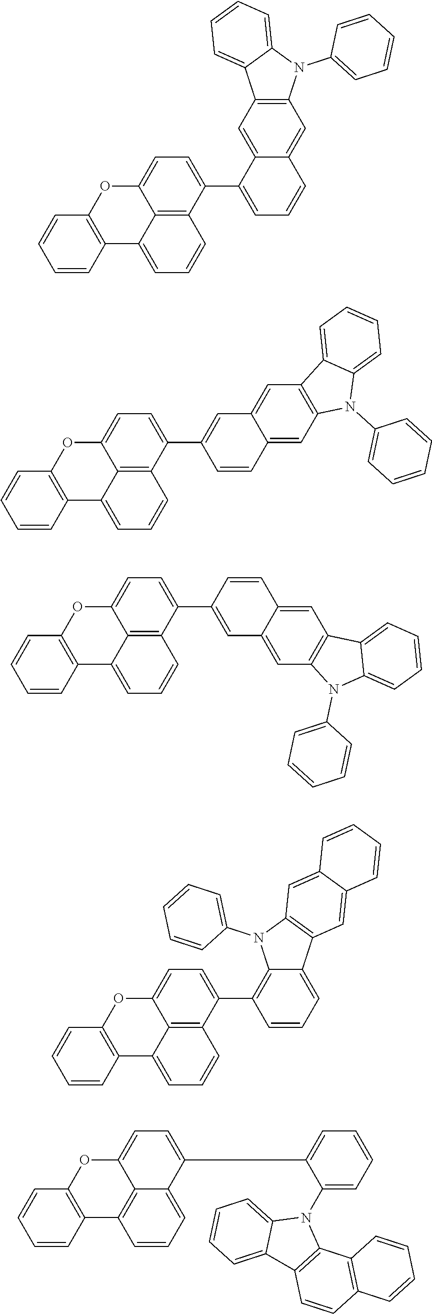

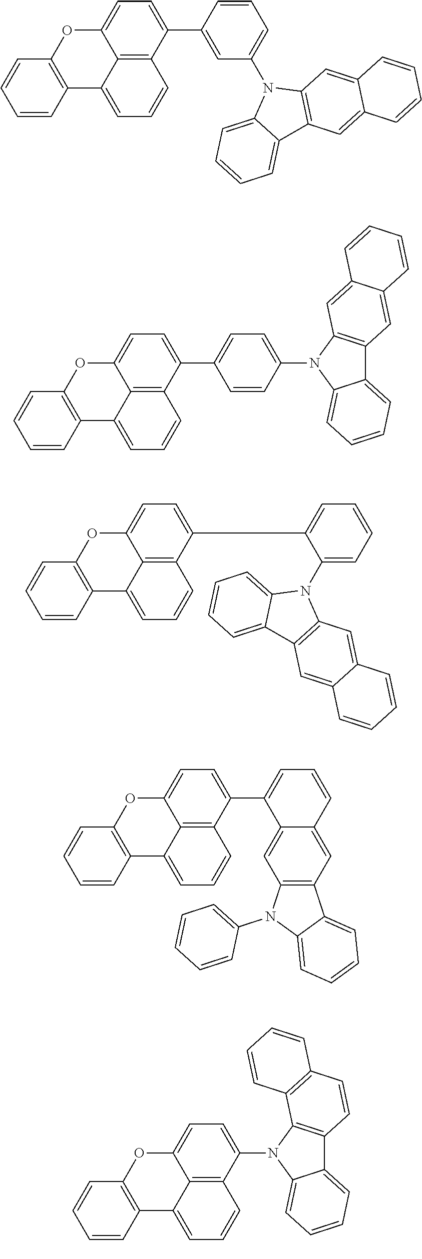

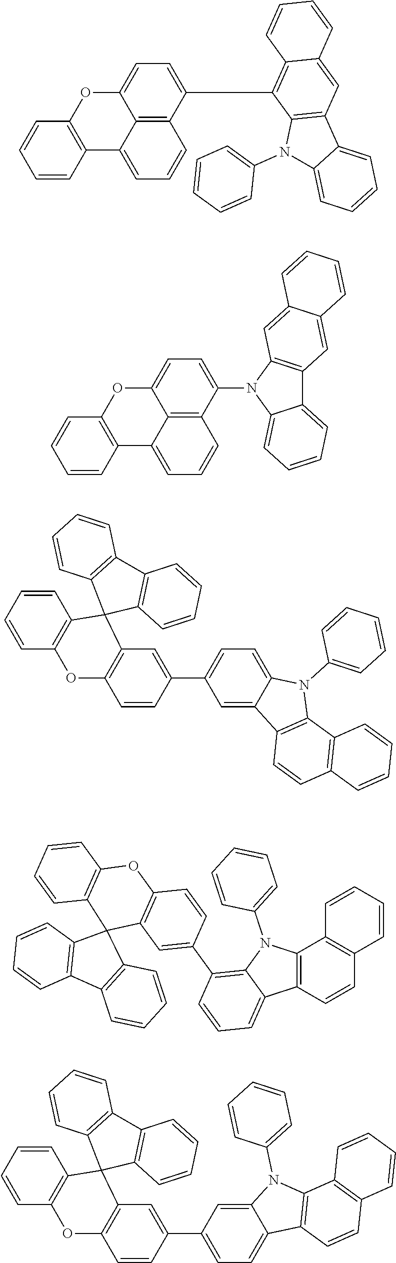

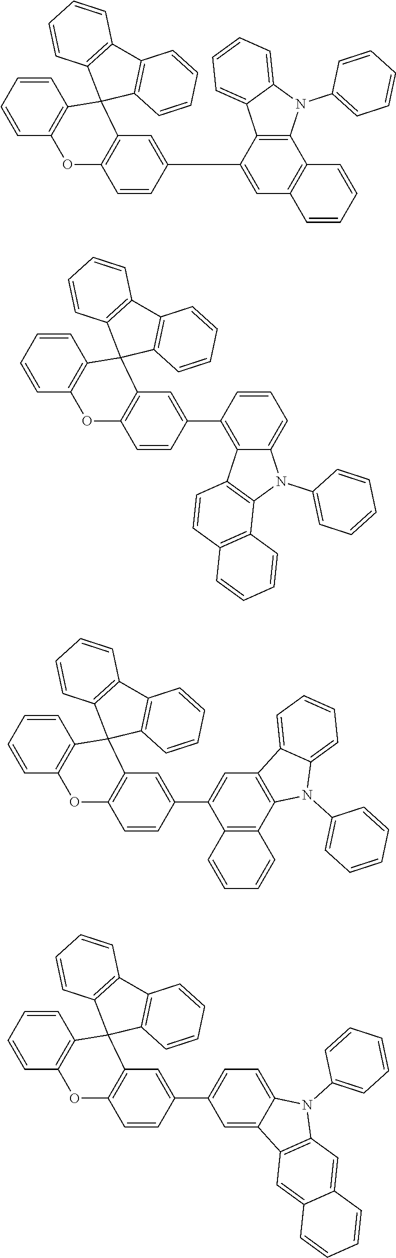 [SMILES: c1ccc(-n2c3ccccc3c3c(-c4ccc5c(c4)C4(c6ccccc6O5)c5ccccc5-c5ccccc54)cc4ccccc4c32)cc1 ; c1ccc(-n2c3cccc(-c4ccc5c(c4)C4(c6ccccc6O5)c5ccccc5-c5ccccc54)c3c3ccc4ccccc4c32)cc1 ; c1ccc(-n2c3ccccc3c3cc(-c4ccc5c(c4)C4(c6ccccc6O5)c5ccccc5-c5ccccc54)c4ccccc4c32)cc1 ; c1ccc(-n2c3ccc(-c4ccc5c(c4)C4(c6ccccc6O5)c5ccccc5-c5ccccc54)cc3c3cc4ccccc4cc32)cc1]

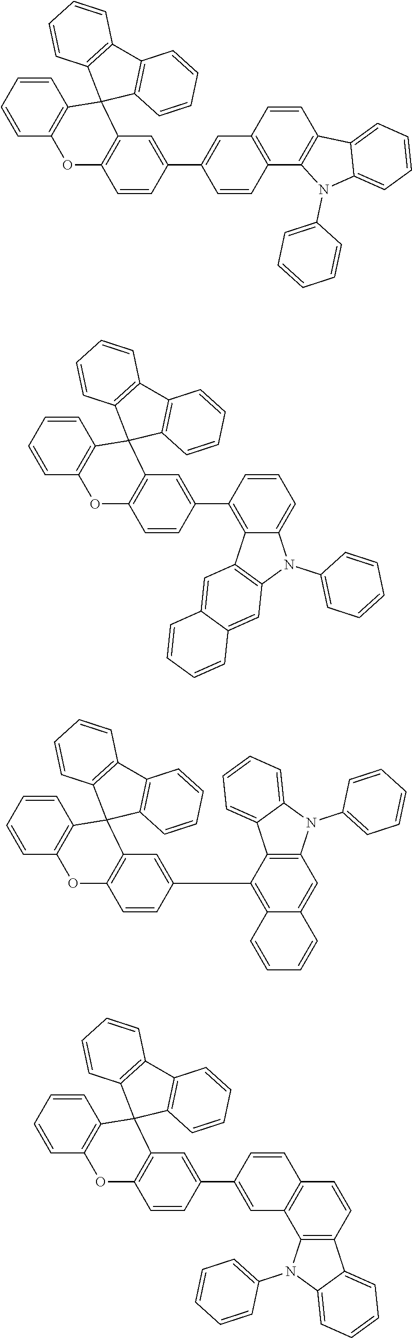 [SMILES: c1ccc(-n2c3ccccc3c3ccc4cc(-c5ccc6c(c5)C5(c7ccccc7O6)c6ccccc6-c6ccccc65)ccc4c32)cc1 ; c1ccc(-n2c3cc4ccccc4cc3c3c(-c4ccc5c(c4)C4(c6ccccc6O5)c5ccccc5-c5ccccc54)cccc32)cc1 ; c1ccc(-n2c3ccccc3c3c(-c4ccc5c(c4)C4(c6ccccc6O5)c5ccccc5-c5ccccc54)c4ccccc4cc32)cc1 ; c1ccc(-n2c3ccccc3c3ccc4ccc(-c5ccc6c(c5)C5(c7ccccc7O6)c6ccccc6-c6ccccc65)cc4c32)cc1]

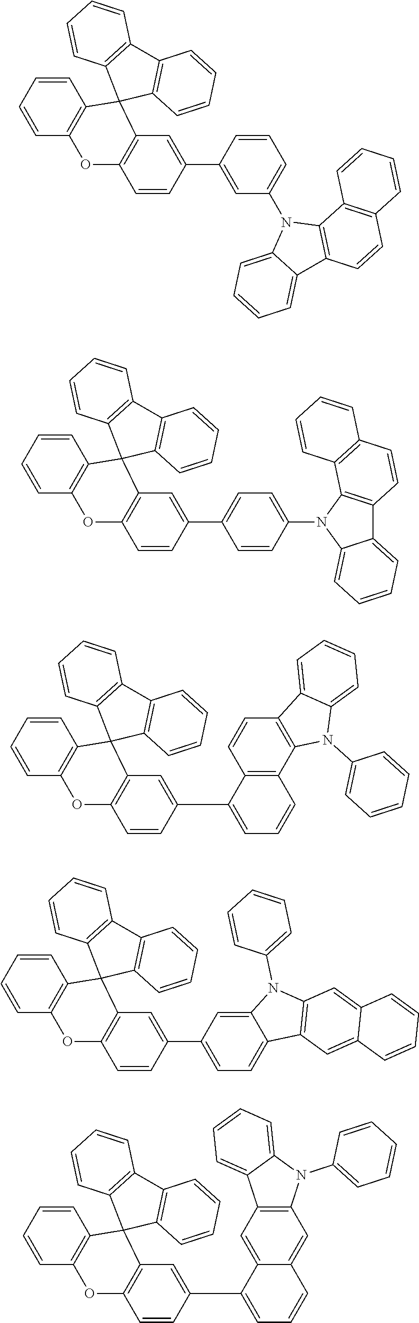

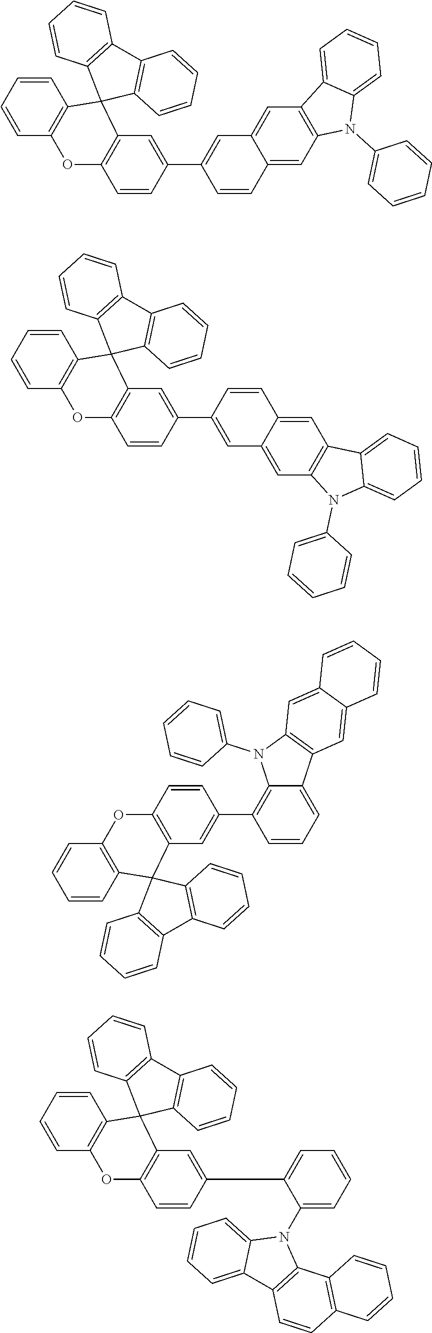

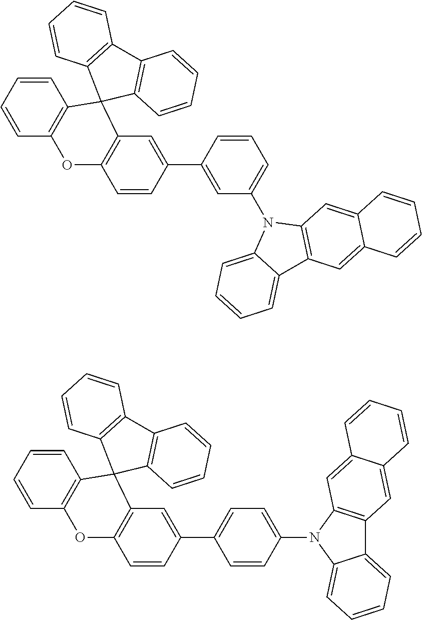 [SMILES: c1cc(-c2ccc3c(c2)C2(c4ccccc4O3)c3ccccc3-c3ccccc32)cc(-n2c3ccccc3c3cc4ccccc4cc32)c1 ; c1ccc2c(c1)Oc1ccc(-c3ccc(-n4c5ccccc5c5cc6ccccc6cc54)cc3)cc1C21c2ccccc2-c2ccccc21]

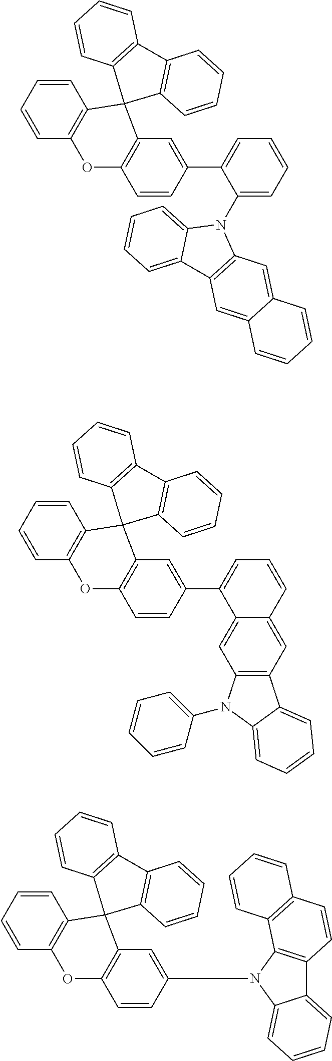 [SMILES: c1ccc2c(c1)Oc1ccc(-c3ccccc3-n3c4ccccc4c4cc5ccccc5cc43)cc1C21c2ccccc2-c2ccccc21 ; c1ccc(-n2c3ccccc3c3cc4cccc(-c5ccc6c(c5)C5(c7ccccc7O6)c6ccccc6-c6ccccc65)c4cc32)cc1 ; c1ccc2c(c1)Oc1ccc(-n3c4ccccc4c4ccc5ccccc5c43)cc1C21c2ccccc2-c2ccccc21]

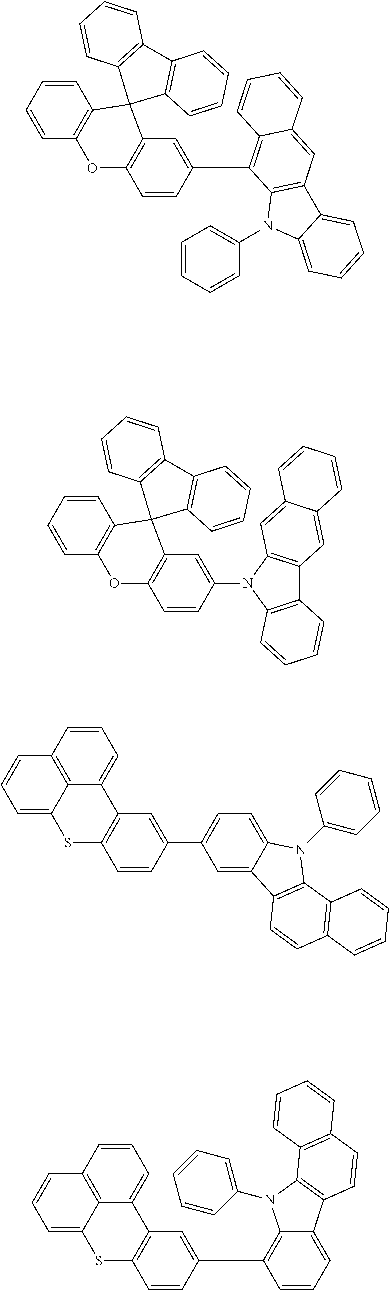 [SMILES: c1ccc(-n2c3ccccc3c3cc4ccccc4c(-c4ccc5c(c4)C4(c6ccccc6O5)c5ccccc5-c5ccccc54)c32)cc1 ; c1ccc2c(c1)Oc1ccc(-n3c4ccccc4c4cc5ccccc5cc43)cc1C21c2ccccc2-c2ccccc21 ; c1ccc(-n2c3ccc(-c4ccc5c(c4)-c4cccc6cccc(c46)S5)cc3c3ccc4ccccc4c32)cc1 ; c1ccc(-n2c3c(-c4ccc5c(c4)-c4cccc6cccc(c46)S5)cccc3c3ccc4ccccc4c32)cc1]

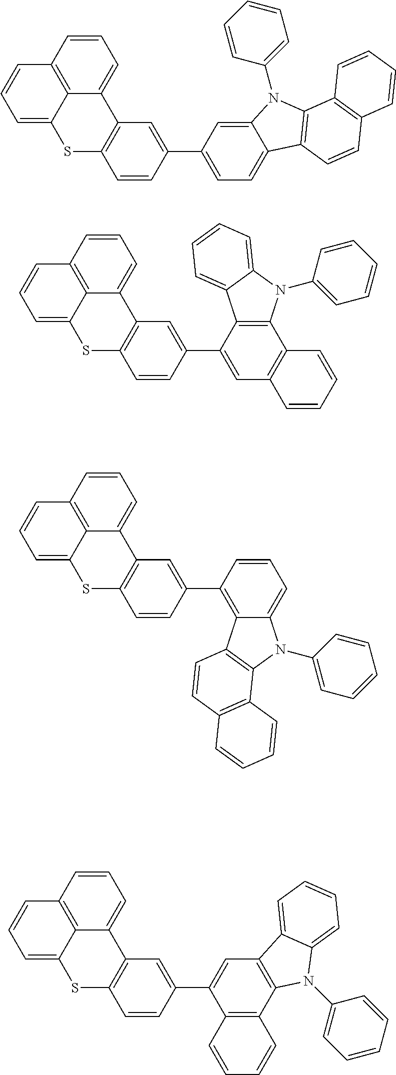

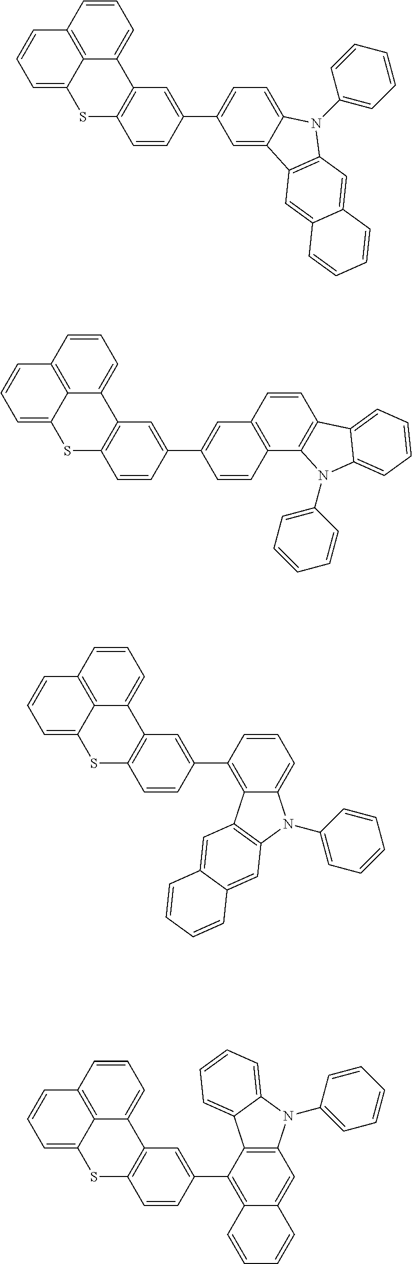 [SMILES: c1ccc(-n2c3ccc(-c4ccc5c(c4)-c4cccc6cccc(c46)S5)cc3c3cc4ccccc4cc32)cc1 ; c1ccc(-n2c3ccccc3c3ccc4cc(-c5ccc6c(c5)-c5cccc7cccc(c57)S6)ccc4c32)cc1 ; c1ccc(-n2c3cc4ccccc4cc3c3c(-c4ccc5c(c4)-c4cccc6cccc(c46)S5)cccc32)cc1 ; c1ccc(-n2c3ccccc3c3c(-c4ccc5c(c4)-c4cccc6cccc(c46)S5)c4ccccc4cc32)cc1]

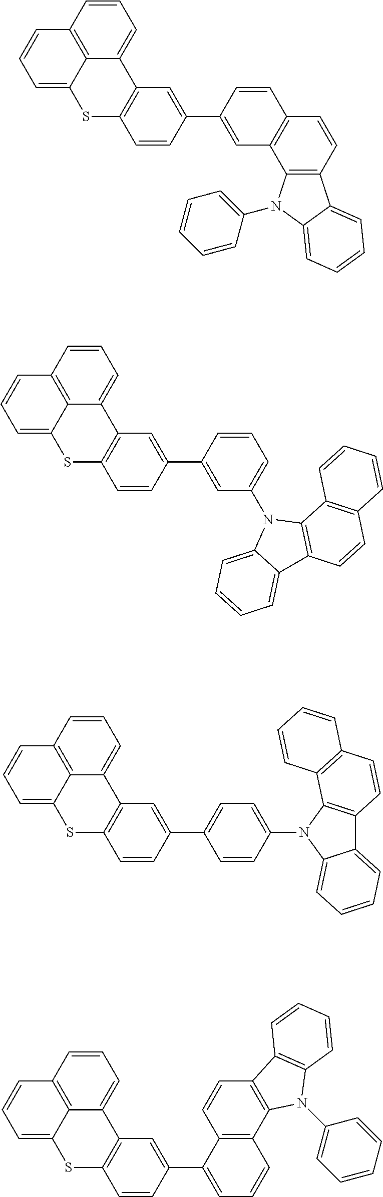 [SMILES: c1ccc(-n2c3ccccc3c3ccc4ccc(-c5ccc6c(c5)-c5cccc7cccc(c57)S6)cc4c32)cc1 ; c1cc(-c2ccc3c(c2)-c2cccc4cccc(c24)S3)cc(-n2c3ccccc3c3ccc4ccccc4c32)c1 ; c1cc2c3c(cccc3c1)-c1cc(-c3ccc(-n4c5ccccc5c5ccc6ccccc6c54)cc3)ccc1S2 ; c1ccc(-n2c3ccccc3c3ccc4c(-c5ccc6c(c5)-c5cccc7cccc(c57)S6)cccc4c32)cc1]

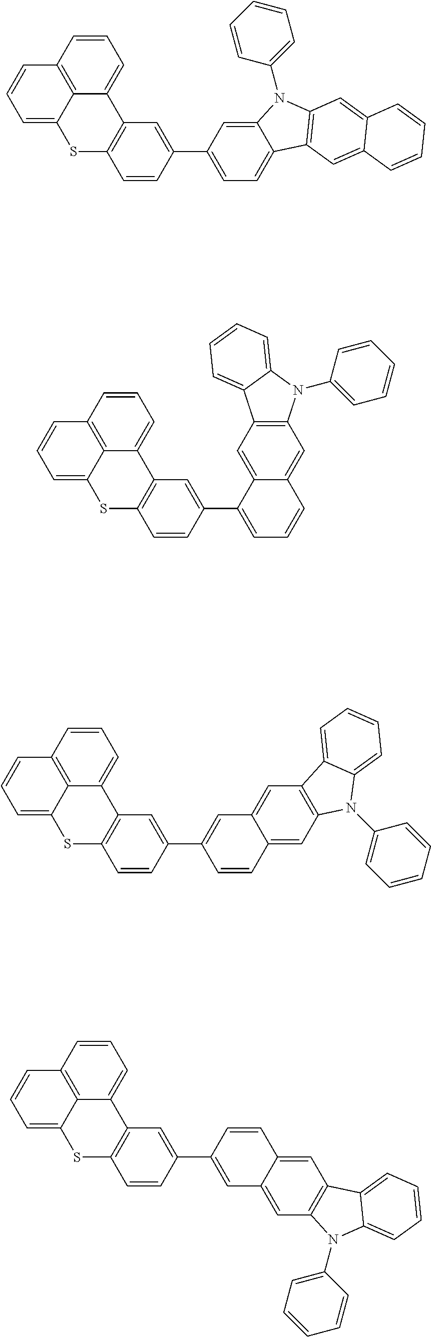 [SMILES: c1ccc(-n2c3cc(-c4ccc5c(c4)-c4cccc6cccc(c46)S5)ccc3c3cc4ccccc4cc32)cc1 ; c1ccc(-n2c3ccccc3c3cc4c(-c5ccc6c(c5)-c5cccc7cccc(c57)S6)cccc4cc32)cc1 ; c1ccc(-n2c3ccccc3c3cc4cc(-c5ccc6c(c5)-c5cccc7cccc(c57)S6)ccc4cc32)cc1 ; c1ccc(-n2c3ccccc3c3cc4ccc(-c5ccc6c(c5)-c5cccc7cccc(c57)S6)cc4cc32)cc1]

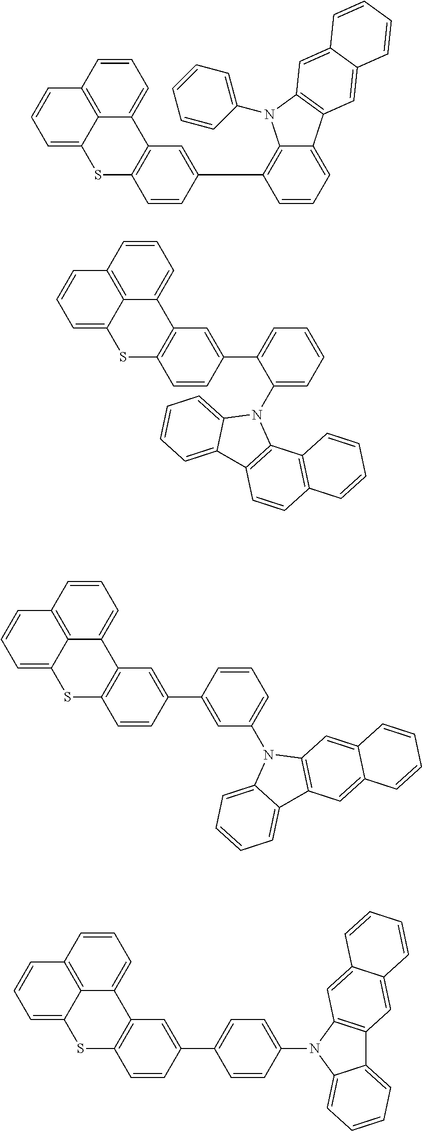

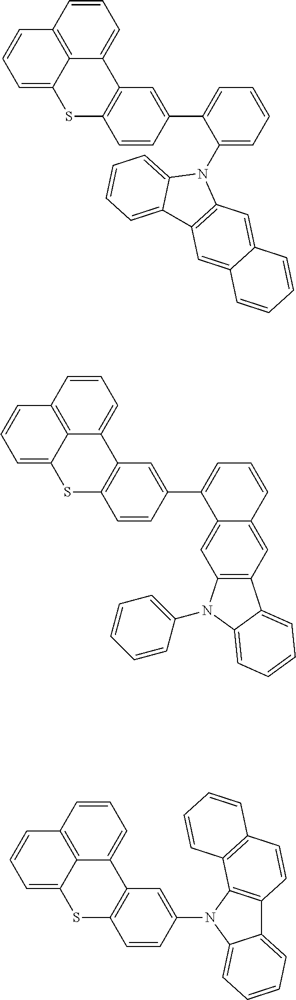 [SMILES: c1ccc(-n2c3ccccc3c3cc4ccccc4cc32)c(-c2ccc3c(c2)-c2cccc4cccc(c24)S3)c1 ; c1ccc(-n2c3ccccc3c3cc4cccc(-c5ccc6c(c5)-c5cccc7cccc(c57)S6)c4cc32)cc1 ; c1cc2c3c(cccc3c1)-c1cc(-n3c4ccccc4c4ccc5ccccc5c43)ccc1S2]

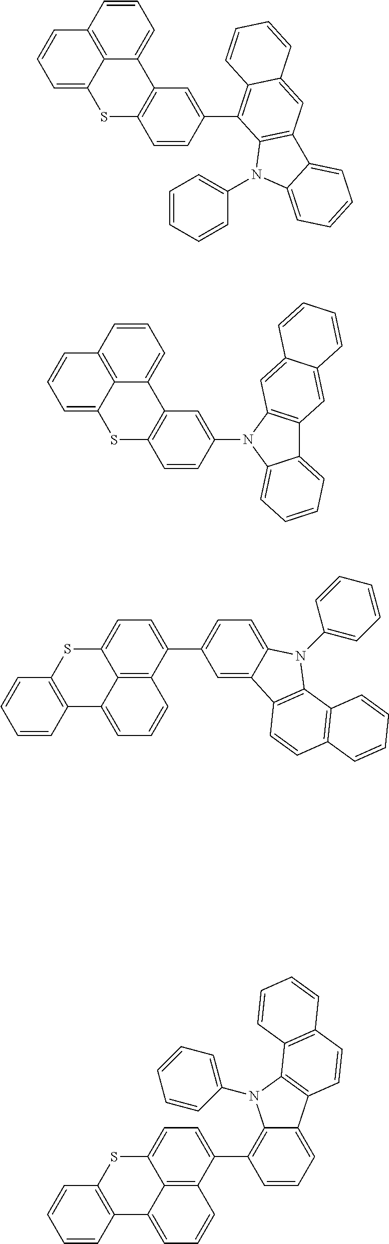 [SMILES: c1ccc(-n2c3ccccc3c3cc4ccccc4c(-c4ccc5c(c4)-c4cccc6cccc(c46)S5)c32)cc1 ; c1ccc2cc3c(cc2c1)c1ccccc1n3-c1ccc2c(c1)-c1cccc3cccc(c13)S2 ; c1ccc(-n2c3ccc(-c4ccc5c6c(cccc46)-c4ccccc4S5)cc3c3ccc4ccccc4c32)cc1 ; c1ccc(-n2c3c(-c4ccc5c6c(cccc46)-c4ccccc4S5)cccc3c3ccc4ccccc4c32)cc1]

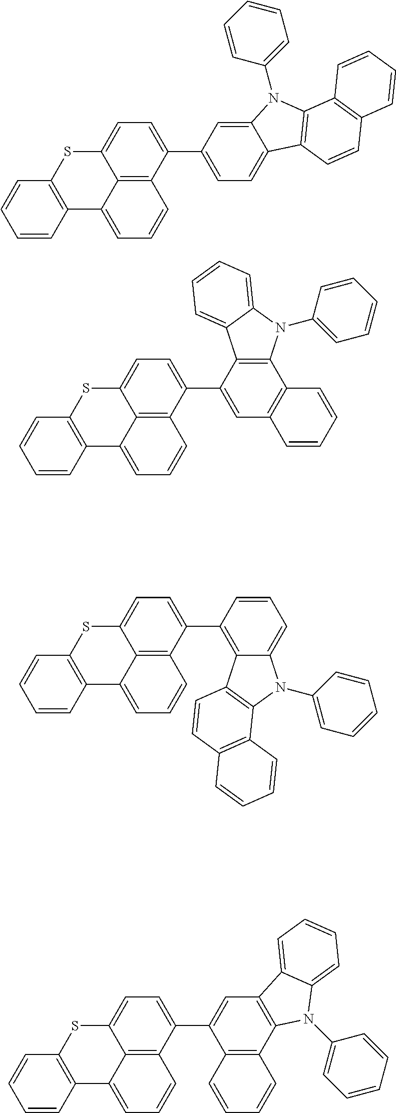 [SMILES: c1ccc(-n2c3cc(-c4ccc5c6c(cccc46)-c4ccccc4S5)ccc3c3ccc4ccccc4c32)cc1 ; c1ccc(-n2c3ccccc3c3c(-c4ccc5c6c(cccc46)-c4ccccc4S5)cc4ccccc4c32)cc1 ; c1ccc(-n2c3cccc(-c4ccc5c6c(cccc46)-c4ccccc4S5)c3c3ccc4ccccc4c32)cc1 ; c1ccc(-n2c3ccccc3c3cc(-c4ccc5c6c(cccc46)-c4ccccc4S5)c4ccccc4c32)cc1]

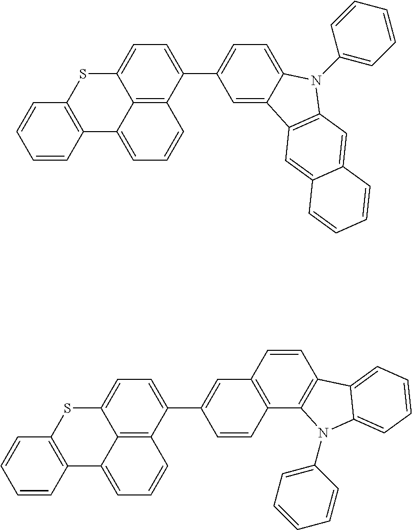 [SMILES: c1ccc(-n2c3ccc(-c4ccc5c6c(cccc46)-c4ccccc4S5)cc3c3cc4ccccc4cc32)cc1 ; c1ccc(-n2c3ccccc3c3ccc4cc(-c5ccc6c7c(cccc57)-c5ccccc5S6)ccc4c32)cc1]

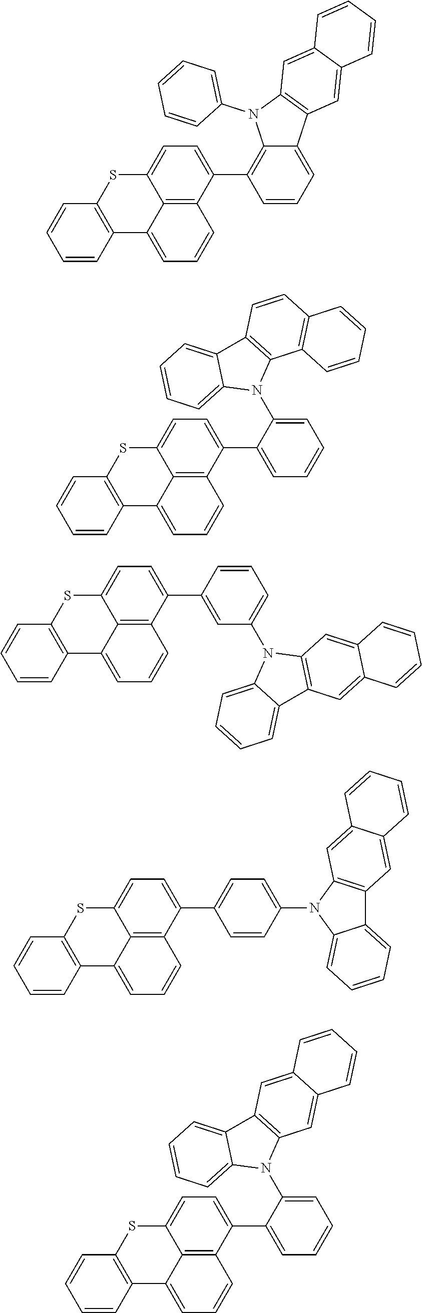

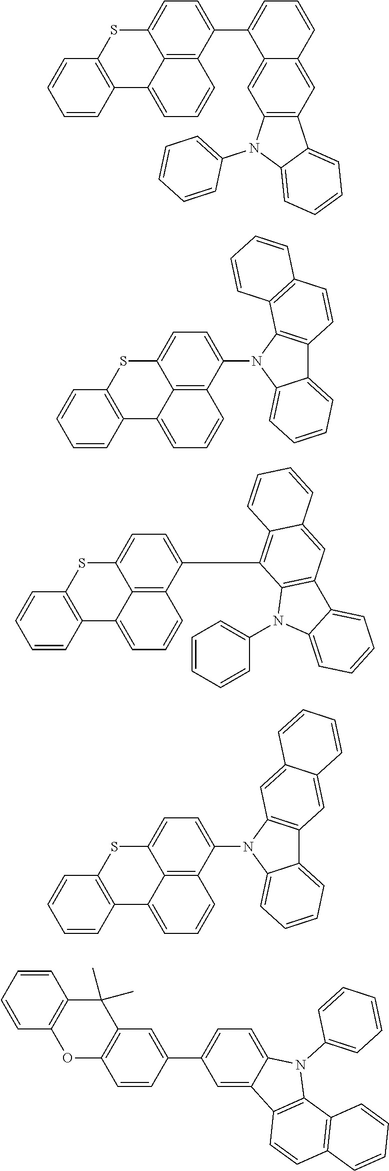

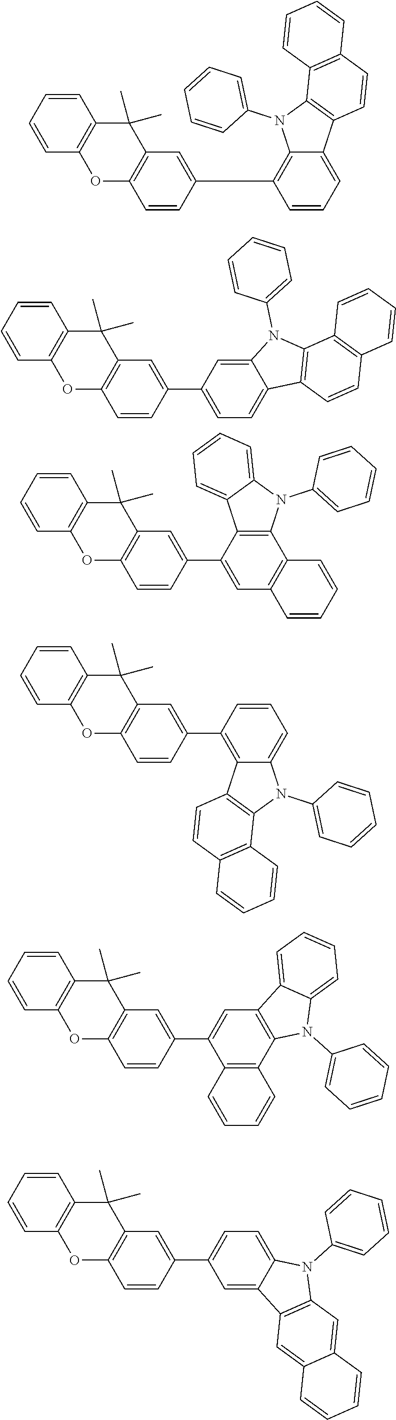

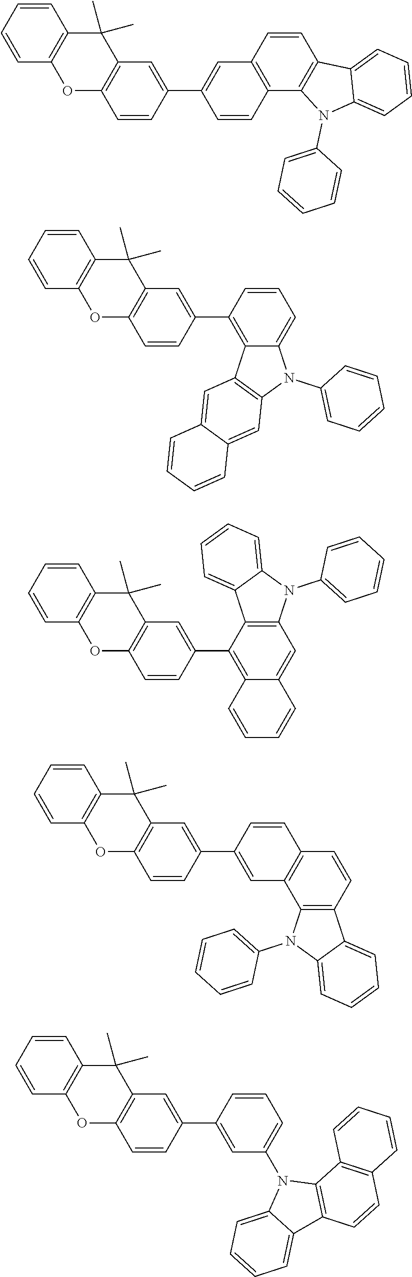 [SMILES: CC1(C)c2ccccc2Oc2ccc(-c3ccc4c(ccc5c6ccccc6n(-c6ccccc6)c45)c3)cc21 ; CC1(C)c2ccccc2Oc2ccc(-c3cccc4c3c3cc5ccccc5cc3n4-c3ccccc3)cc21 ; CC1(C)c2ccccc2Oc2ccc(-c3c4ccccc4cc4c3c3ccccc3n4-c3ccccc3)cc21 ; CC1(C)c2ccccc2Oc2ccc(-c3ccc4ccc5c6ccccc6n(-c6ccccc6)c5c4c3)cc21 ; CC1(C)c2ccccc2Oc2ccc(-c3cccc(-n4c5ccccc5c5ccc6ccccc6c54)c3)cc21]

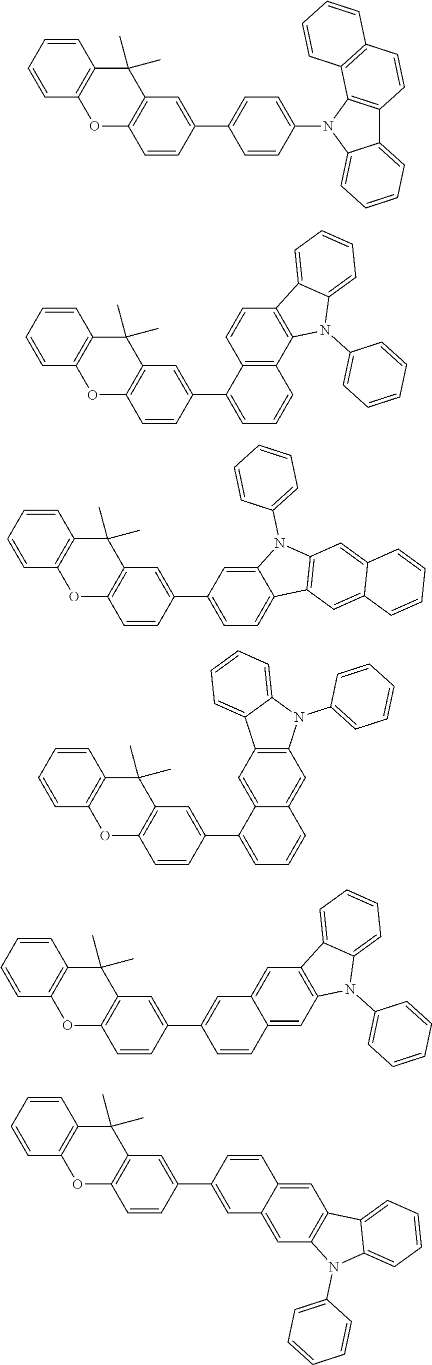

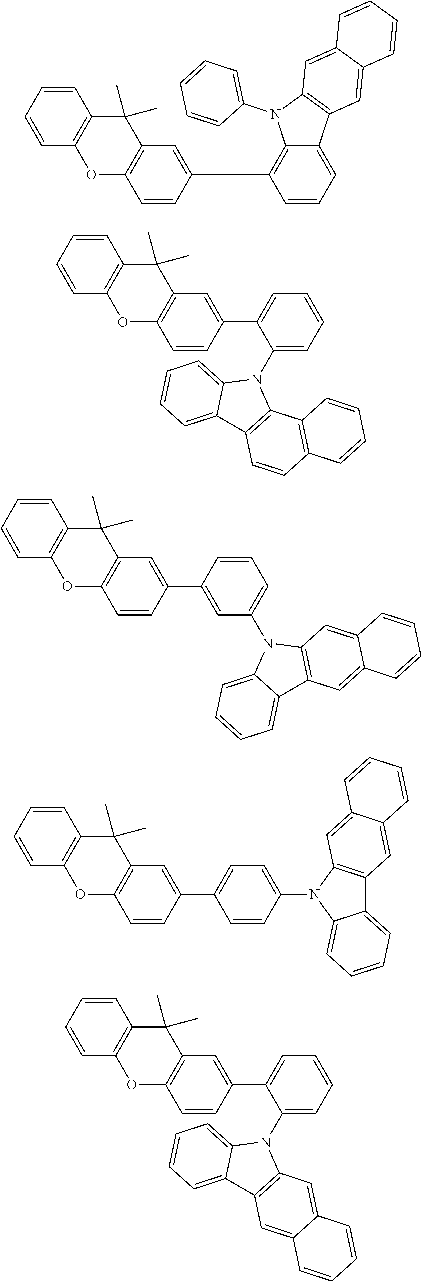

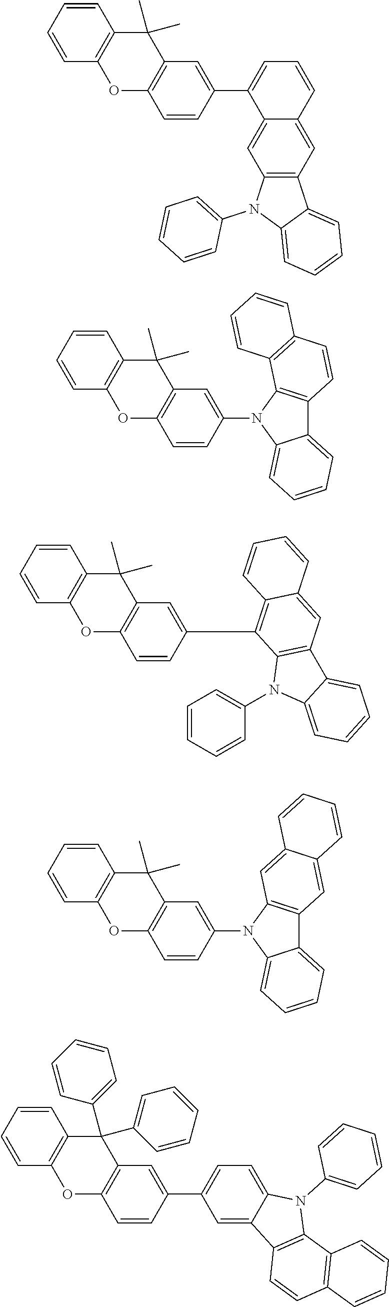

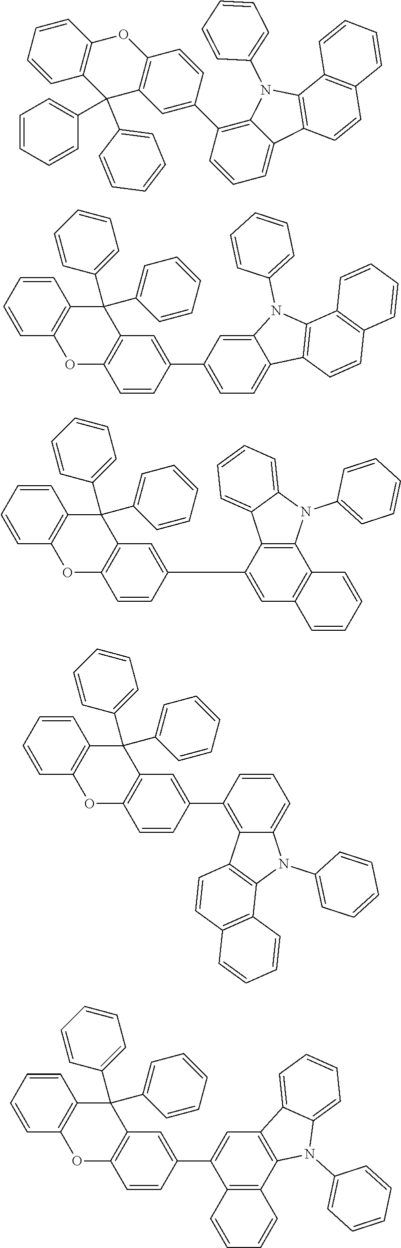

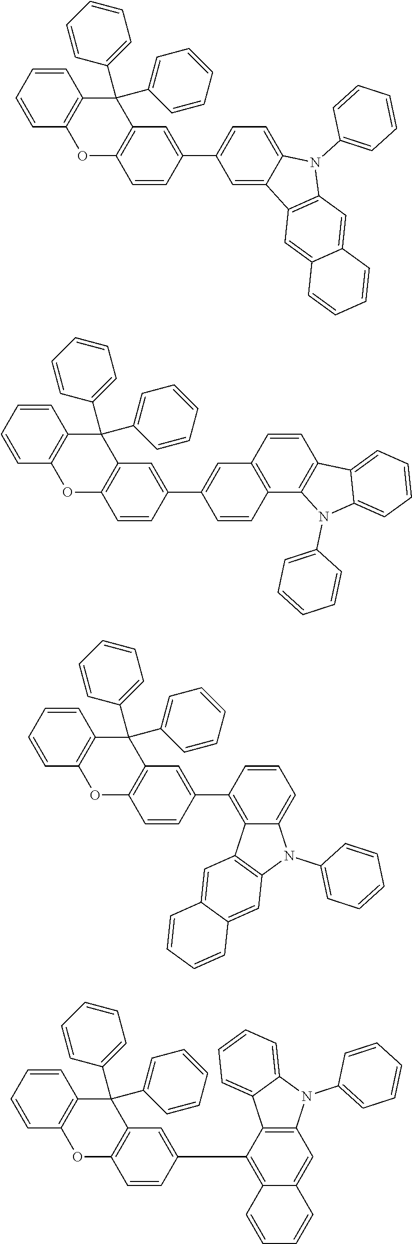 [SMILES: c1ccc(-n2c3ccc(-c4ccc5c(c4)C(c4ccccc4)(c4ccccc4)c4ccccc4O5)cc3c3cc4ccccc4cc32)cc1 ; c1ccc(-n2c3ccccc3c3ccc4cc(-c5ccc6c(c5)C(c5ccccc5)(c5ccccc5)c5ccccc5O6)ccc4c32)cc1 ; c1ccc(-n2c3cc4ccccc4cc3c3c(-c4ccc5c(c4)C(c4ccccc4)(c4ccccc4)c4ccccc4O5)cccc32)cc1 ; c1ccc(-n2c3ccccc3c3c(-c4ccc5c(c4)C(c4ccccc4)(c4ccccc4)c4ccccc4O5)c4ccccc4cc32)cc1]

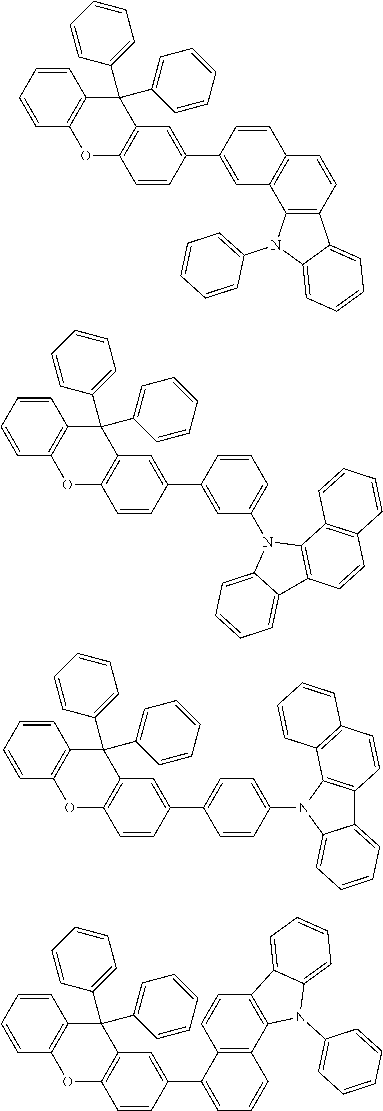

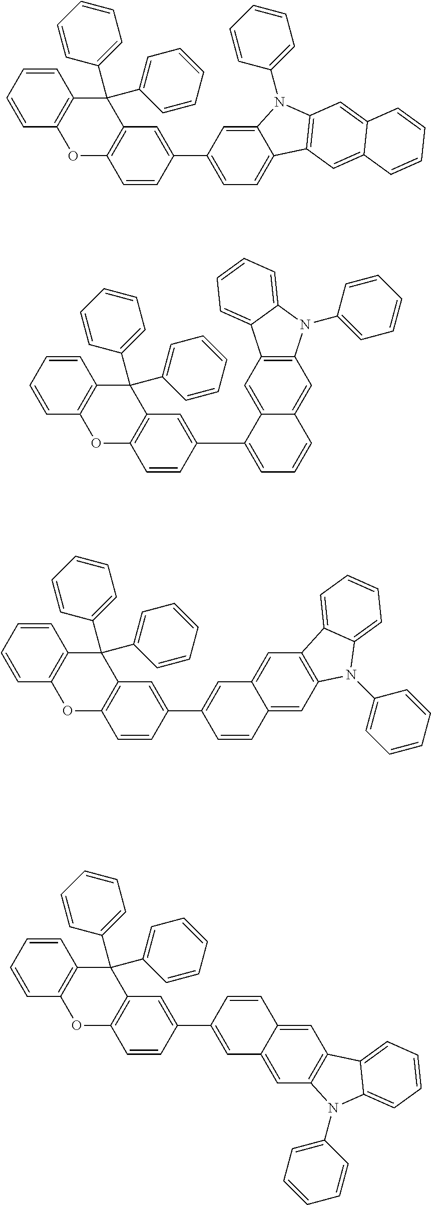 [SMILES: c1ccc(-n2c3cc(-c4ccc5c(c4)C(c4ccccc4)(c4ccccc4)c4ccccc4O5)ccc3c3cc4ccccc4cc32)cc1 ; c1ccc(-n2c3ccccc3c3cc4c(-c5ccc6c(c5)C(c5ccccc5)(c5ccccc5)c5ccccc5O6)cccc4cc32)cc1 ; c1ccc(-n2c3ccccc3c3cc4cc(-c5ccc6c(c5)C(c5ccccc5)(c5ccccc5)c5ccccc5O6)ccc4cc32)cc1 ; c1ccc(-n2c3ccccc3c3cc4ccc(-c5ccc6c(c5)C(c5ccccc5)(c5ccccc5)c5ccccc5O6)cc4cc32)cc1]

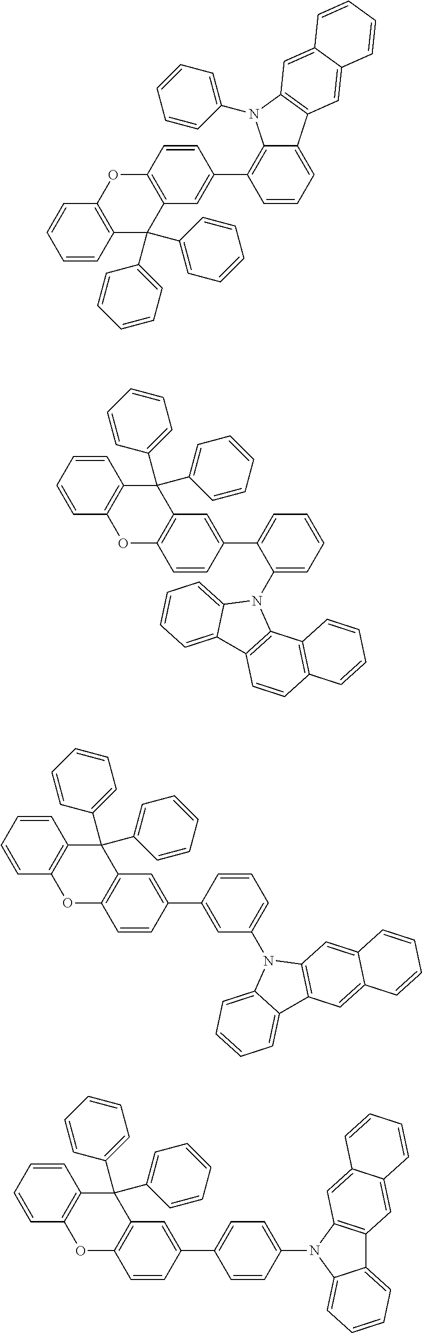 [SMILES: c1ccc(-n2c3cc4ccccc4cc3c3cccc(-c4ccc5c(c4)C(c4ccccc4)(c4ccccc4)c4ccccc4O5)c32)cc1 ; c1ccc(C2(c3ccccc3)c3ccccc3Oc3ccc(-c4ccccc4-n4c5ccccc5c5ccc6ccccc6c54)cc32)cc1 ; c1ccc(C2(c3ccccc3)c3ccccc3Oc3ccc(-c4cccc(-n5c6ccccc6c6cc7ccccc7cc65)c4)cc32)cc1 ; c1ccc(C2(c3ccccc3)c3ccccc3Oc3ccc(-c4ccc(-n5c6ccccc6c6cc7ccccc7cc65)cc4)cc32)cc1]

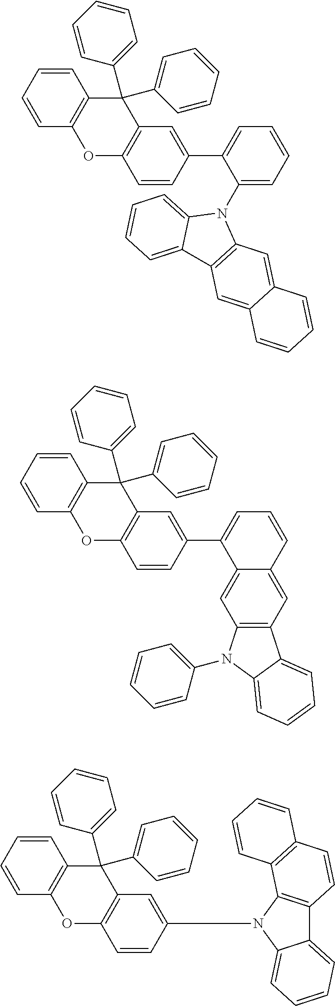 [SMILES: c1ccc(C2(c3ccccc3)c3ccccc3Oc3ccc(-c4ccccc4-n4c5ccccc5c5cc6ccccc6cc54)cc32)cc1 ; c1ccc(-n2c3ccccc3c3cc4cccc(-c5ccc6c(c5)C(c5ccccc5)(c5ccccc5)c5ccccc5O6)c4cc32)cc1 ; c1ccc(C2(c3ccccc3)c3ccccc3Oc3ccc(-n4c5ccccc5c5ccc6ccccc6c54)cc32)cc1]

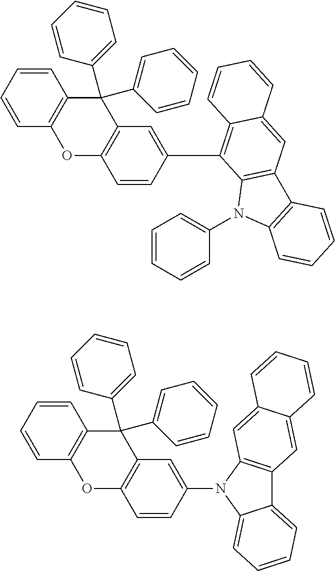 [SMILES: c1ccc(-n2c3ccccc3c3cc4ccccc4c(-c4ccc5c(c4)C(c4ccccc4)(c4ccccc4)c4ccccc4O5)c32)cc1 ; c1ccc(C2(c3ccccc3)c3ccccc3Oc3ccc(-n4c5ccccc5c5cc6ccccc6cc54)cc32)cc1]

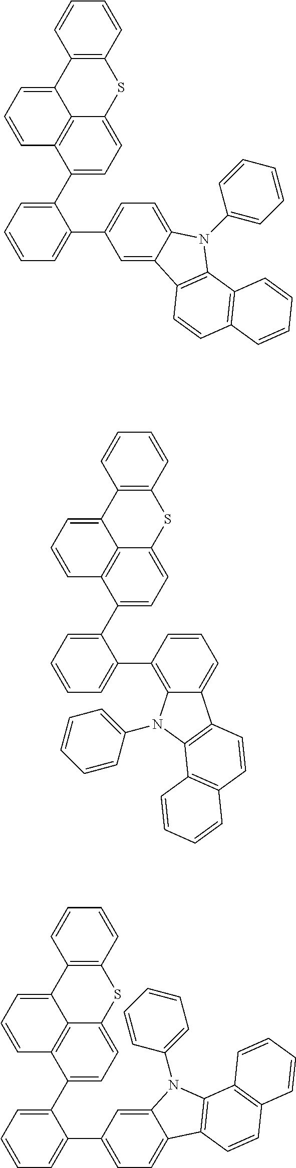 [SMILES: c1ccc(-n2c3ccc(-c4ccccc4-c4ccc5c6c(cccc46)-c4ccccc4S5)cc3c3ccc4ccccc4c32)cc1 ; c1ccc(-n2c3c(-c4ccccc4-c4ccc5c6c(cccc46)-c4ccccc4S5)cccc3c3ccc4ccccc4c32)cc1 ; c1ccc(-n2c3cc(-c4ccccc4-c4ccc5c6c(cccc46)-c4ccccc4S5)ccc3c3ccc4ccccc4c32)cc1]

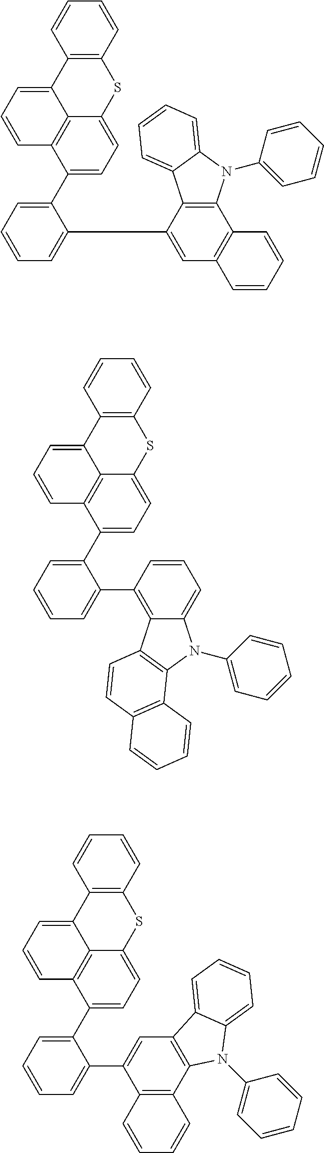 [SMILES: c1ccc(-n2c3ccccc3c3c(-c4ccccc4-c4ccc5c6c(cccc46)-c4ccccc4S5)cc4ccccc4c32)cc1 ; c1ccc(-n2c3cccc(-c4ccccc4-c4ccc5c6c(cccc46)-c4ccccc4S5)c3c3ccc4ccccc4c32)cc1 ; c1ccc(-n2c3ccccc3c3cc(-c4ccccc4-c4ccc5c6c(cccc46)-c4ccccc4S5)c4ccccc4c32)cc1]

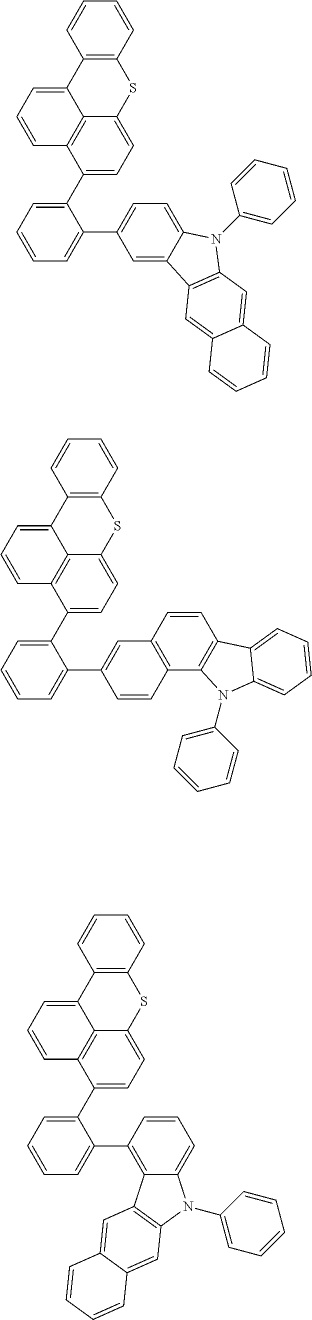 [SMILES: c1ccc(-n2c3ccc(-c4ccccc4-c4ccc5c6c(cccc46)-c4ccccc4S5)cc3c3cc4ccccc4cc32)cc1 ; c1ccc(-n2c3ccccc3c3ccc4cc(-c5ccccc5-c5ccc6c7c(cccc57)-c5ccccc5S6)ccc4c32)cc1 ; c1ccc(-n2c3cc4ccccc4cc3c3c(-c4ccccc4-c4ccc5c6c(cccc46)-c4ccccc4S5)cccc32)cc1]

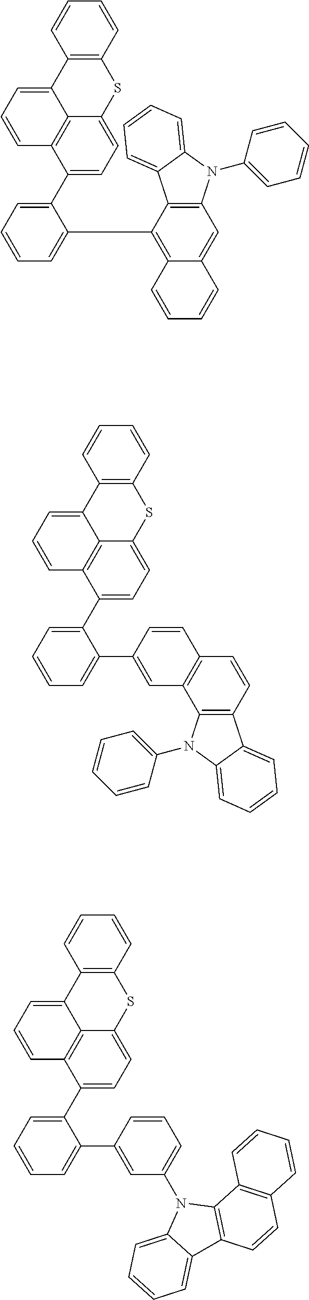 [SMILES: c1ccc(-n2c3ccccc3c3c(-c4ccccc4-c4ccc5c6c(cccc46)-c4ccccc4S5)c4ccccc4cc32)cc1 ; c1ccc(-n2c3ccccc3c3ccc4ccc(-c5ccccc5-c5ccc6c7c(cccc57)-c5ccccc5S6)cc4c32)cc1 ; c1cc(-c2ccccc2-c2ccc3c4c(cccc24)-c2ccccc2S3)cc(-n2c3ccccc3c3ccc4ccccc4c32)c1]

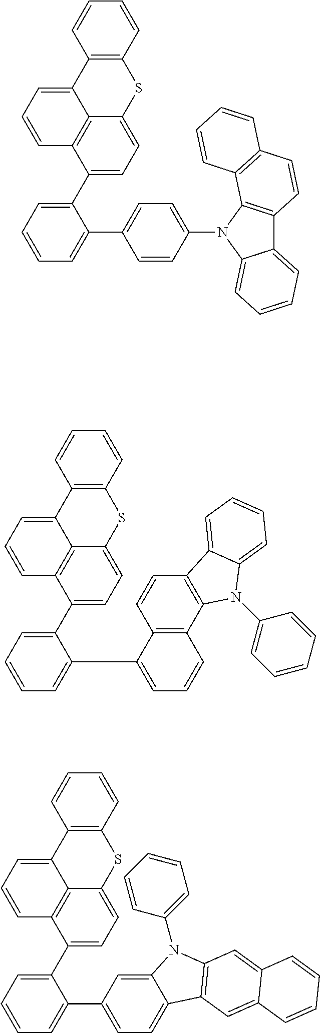 [SMILES: c1ccc2c(c1)Sc1ccc(-c3ccccc3-c3ccc(-n4c5ccccc5c5ccc6ccccc6c54)cc3)c3cccc-2c13 ; c1ccc(-n2c3ccccc3c3ccc4c(-c5ccccc5-c5ccc6c7c(cccc57)-c5ccccc5S6)cccc4c32)cc1 ; c1ccc(-n2c3cc(-c4ccccc4-c4ccc5c6c(cccc46)-c4ccccc4S5)ccc3c3cc4ccccc4cc32)cc1]

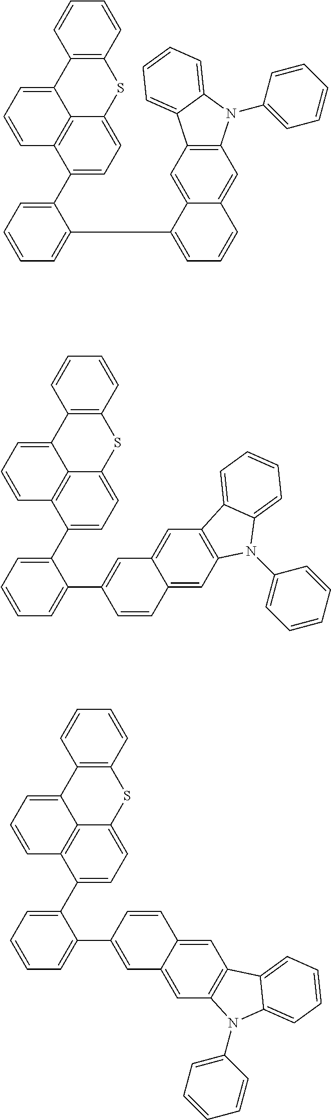 [SMILES: c1ccc(-n2c3ccccc3c3cc4c(-c5ccccc5-c5ccc6c7c(cccc57)-c5ccccc5S6)cccc4cc32)cc1 ; c1ccc(-n2c3ccccc3c3cc4cc(-c5ccccc5-c5ccc6c7c(cccc57)-c5ccccc5S6)ccc4cc32)cc1 ; c1ccc(-n2c3ccccc3c3cc4ccc(-c5ccccc5-c5ccc6c7c(cccc57)-c5ccccc5S6)cc4cc32)cc1]

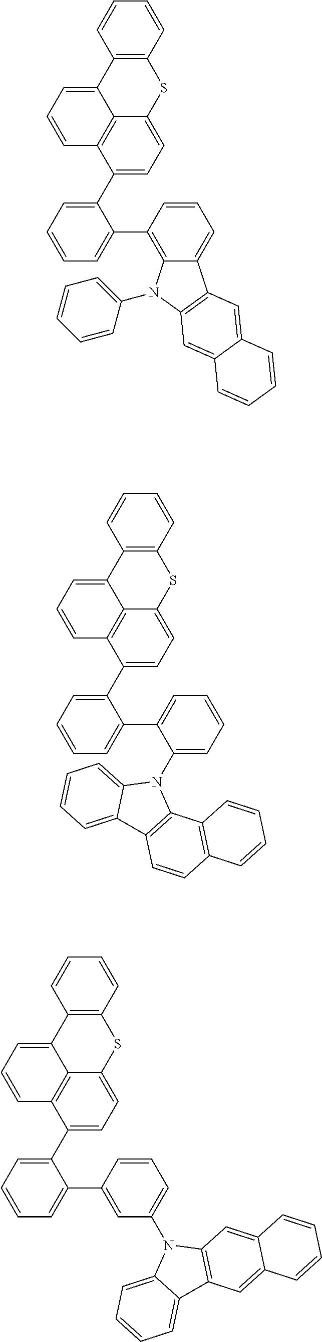 [SMILES: c1ccc(-n2c3cc4ccccc4cc3c3cccc(-c4ccccc4-c4ccc5c6c(cccc46)-c4ccccc4S5)c32)cc1 ; c1ccc2c(c1)Sc1ccc(-c3ccccc3-c3ccccc3-n3c4ccccc4c4ccc5ccccc5c43)c3cccc-2c13 ; c1cc(-c2ccccc2-c2ccc3c4c(cccc24)-c2ccccc2S3)cc(-n2c3ccccc3c3cc4ccccc4cc32)c1]

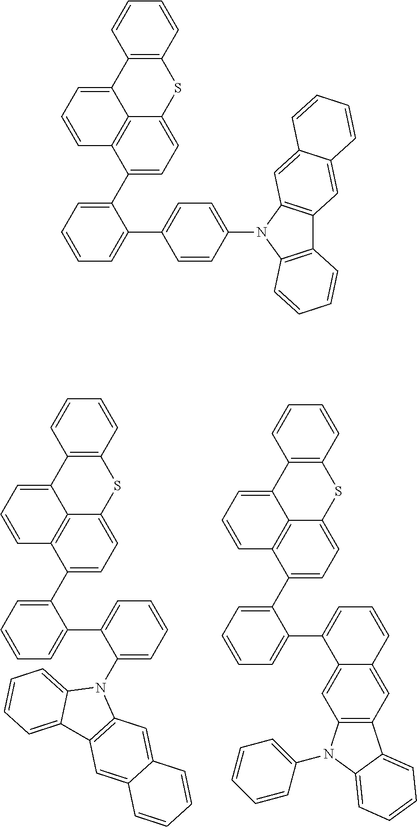 [SMILES: c1ccc2c(c1)Sc1ccc(-c3ccccc3-c3ccc(-n4c5ccccc5c5cc6ccccc6cc54)cc3)c3cccc-2c13 ; c1ccc2c(c1)Sc1ccc(-c3ccccc3-c3ccccc3-n3c4ccccc4c4cc5ccccc5cc43)c3cccc-2c13 ; c1ccc(-n2c3ccccc3c3cc4cccc(-c5ccccc5-c5ccc6c7c(cccc57)-c5ccccc5S6)c4cc32)cc1]

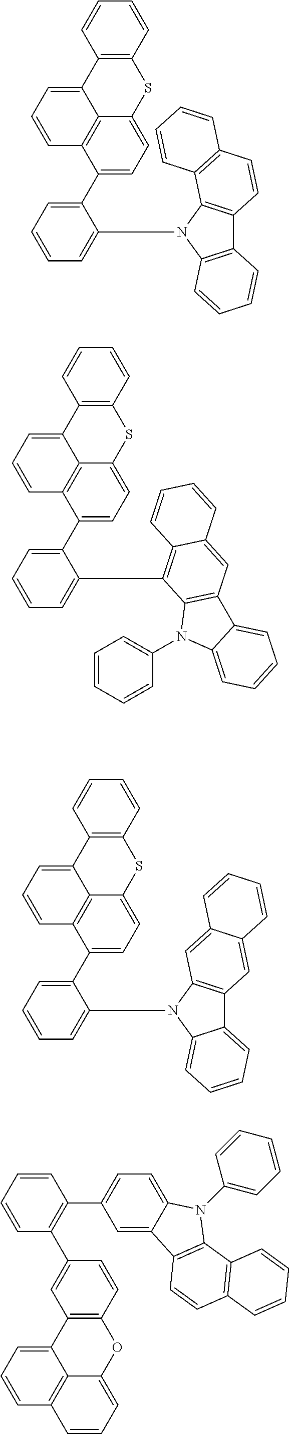 [SMILES: c1ccc2c(c1)Sc1ccc(-c3ccccc3-n3c4ccccc4c4ccc5ccccc5c43)c3cccc-2c13 ; c1ccc(-n2c3ccccc3c3cc4ccccc4c(-c4ccccc4-c4ccc5c6c(cccc46)-c4ccccc4S5)c32)cc1 ; c1ccc2c(c1)Sc1ccc(-c3ccccc3-n3c4ccccc4c4cc5ccccc5cc43)c3cccc-2c13 ; c1ccc(-n2c3ccc(-c4ccccc4-c4ccc5c(c4)-c4cccc6cccc(c46)O5)cc3c3ccc4ccccc4c32)cc1]

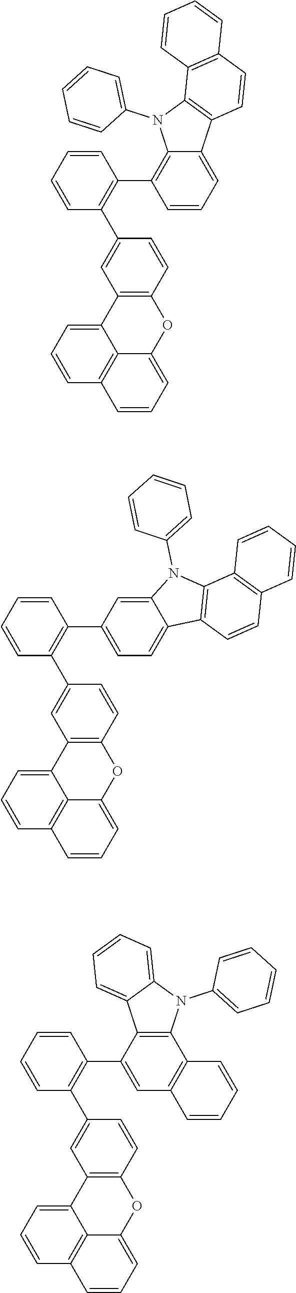 [SMILES: c1ccc(-n2c3c(-c4ccccc4-c4ccc5c(c4)-c4cccc6cccc(c46)O5)cccc3c3ccc4ccccc4c32)cc1 ; c1ccc(-n2c3cc(-c4ccccc4-c4ccc5c(c4)-c4cccc6cccc(c46)O5)ccc3c3ccc4ccccc4c32)cc1 ; c1ccc(-n2c3ccccc3c3c(-c4ccccc4-c4ccc5c(c4)-c4cccc6cccc(c46)O5)cc4ccccc4c32)cc1]

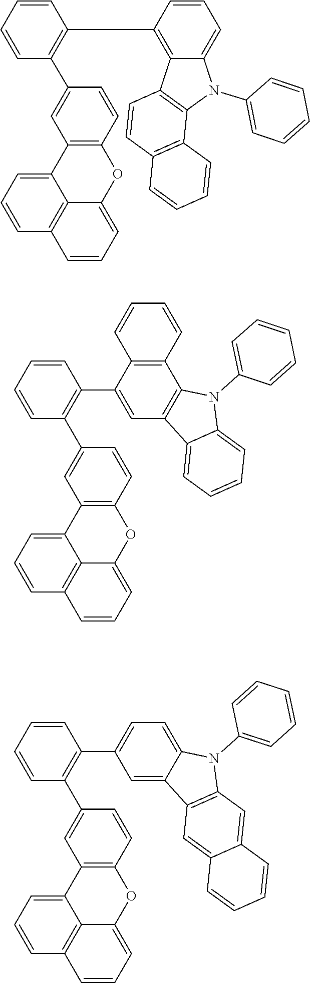 [SMILES: c1ccc(-n2c3cccc(-c4ccccc4-c4ccc5c(c4)-c4cccc6cccc(c46)O5)c3c3ccc4ccccc4c32)cc1 ; c1ccc(-n2c3ccccc3c3cc(-c4ccccc4-c4ccc5c(c4)-c4cccc6cccc(c46)O5)c4ccccc4c32)cc1 ; c1ccc(-n2c3ccc(-c4ccccc4-c4ccc5c(c4)-c4cccc6cccc(c46)O5)cc3c3cc4ccccc4cc32)cc1]

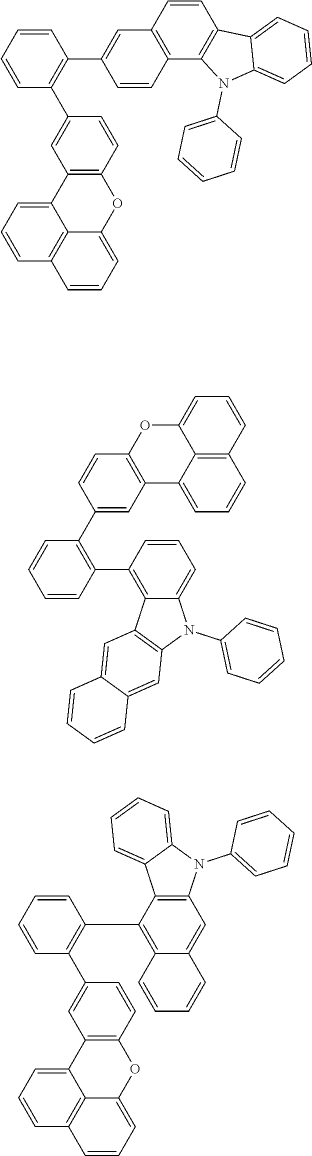 [SMILES: c1ccc(-n2c3ccccc3c3ccc4cc(-c5ccccc5-c5ccc6c(c5)-c5cccc7cccc(c57)O6)ccc4c32)cc1 ; c1ccc(-n2c3cc4ccccc4cc3c3c(-c4ccccc4-c4ccc5c(c4)-c4cccc6cccc(c46)O5)cccc32)cc1 ; c1ccc(-n2c3ccccc3c3c(-c4ccccc4-c4ccc5c(c4)-c4cccc6cccc(c46)O5)c4ccccc4cc32)cc1]

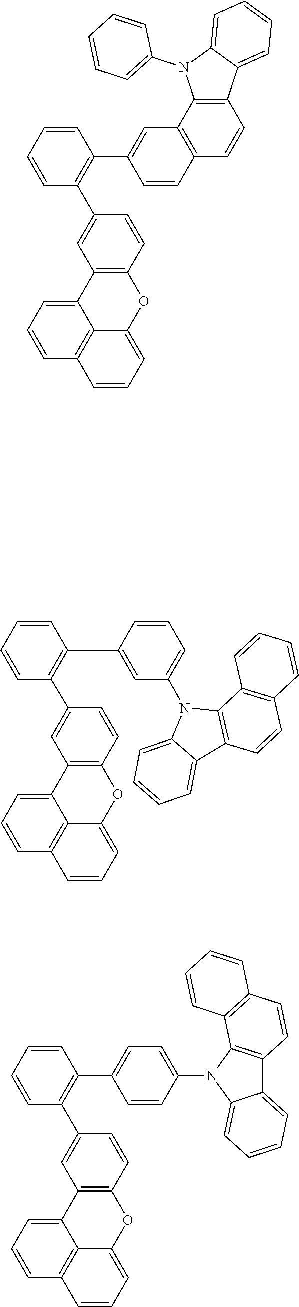 [SMILES: c1ccc(-n2c3ccccc3c3ccc4ccc(-c5ccccc5-c5ccc6c(c5)-c5cccc7cccc(c57)O6)cc4c32)cc1 ; c1cc(-c2ccccc2-c2ccc3c(c2)-c2cccc4cccc(c24)O3)cc(-n2c3ccccc3c3ccc4ccccc4c32)c1 ; c1ccc(-c2ccc3c(c2)-c2cccc4cccc(c24)O3)c(-c2ccc(-n3c4ccccc4c4ccc5ccccc5c43)cc2)c1]

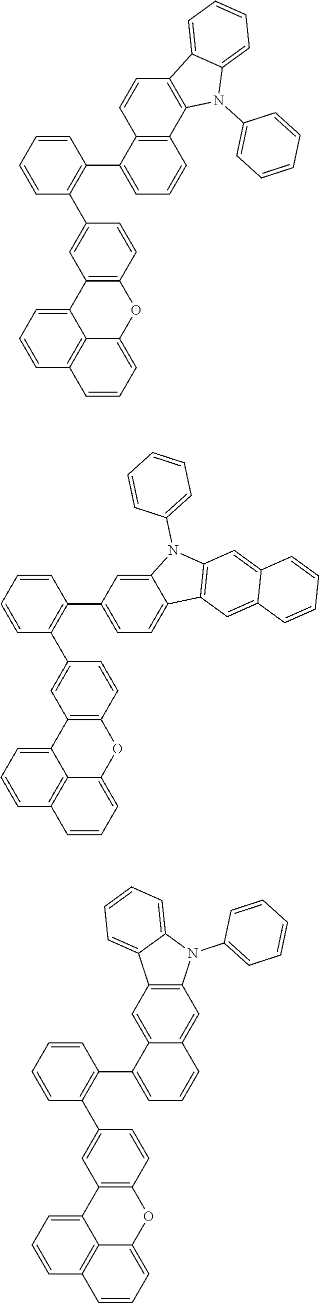 [SMILES: c1ccc(-n2c3ccccc3c3ccc4c(-c5ccccc5-c5ccc6c(c5)-c5cccc7cccc(c57)O6)cccc4c32)cc1 ; c1ccc(-n2c3cc(-c4ccccc4-c4ccc5c(c4)-c4cccc6cccc(c46)O5)ccc3c3cc4ccccc4cc32)cc1 ; c1ccc(-n2c3ccccc3c3cc4c(-c5ccccc5-c5ccc6c(c5)-c5cccc7cccc(c57)O6)cccc4cc32)cc1]

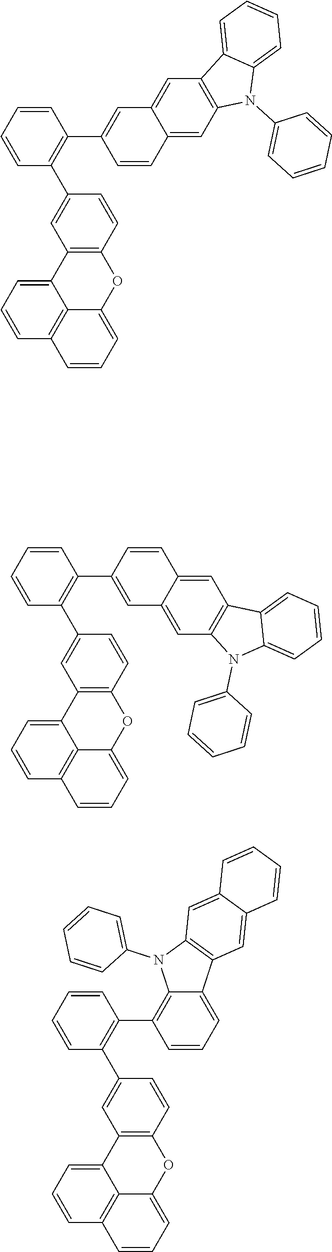 [SMILES: c1ccc(-n2c3ccccc3c3cc4cc(-c5ccccc5-c5ccc6c(c5)-c5cccc7cccc(c57)O6)ccc4cc32)cc1 ; c1ccc(-n2c3ccccc3c3cc4ccc(-c5ccccc5-c5ccc6c(c5)-c5cccc7cccc(c57)O6)cc4cc32)cc1 ; c1ccc(-n2c3cc4ccccc4cc3c3cccc(-c4ccccc4-c4ccc5c(c4)-c4cccc6cccc(c46)O5)c32)cc1]

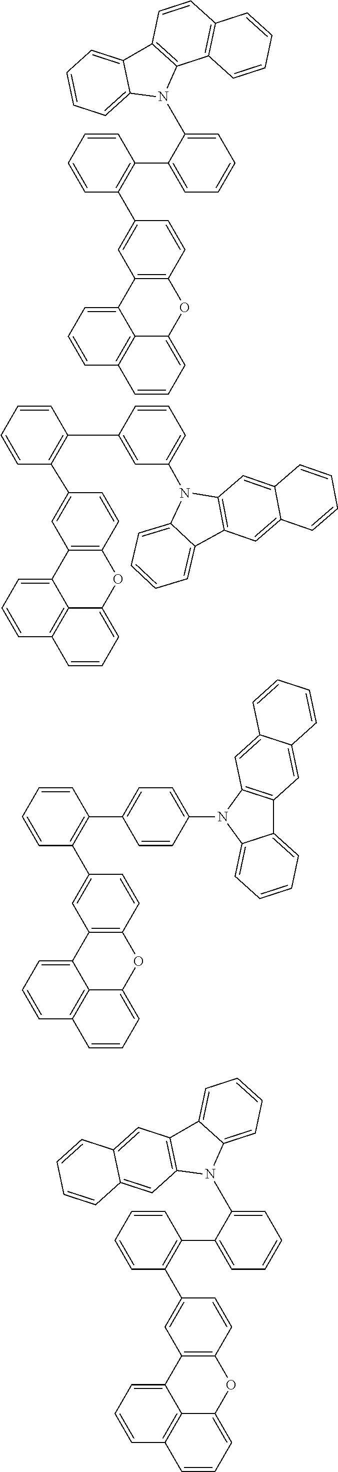

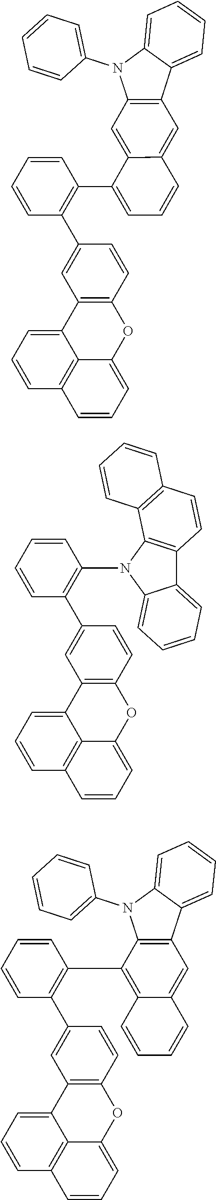

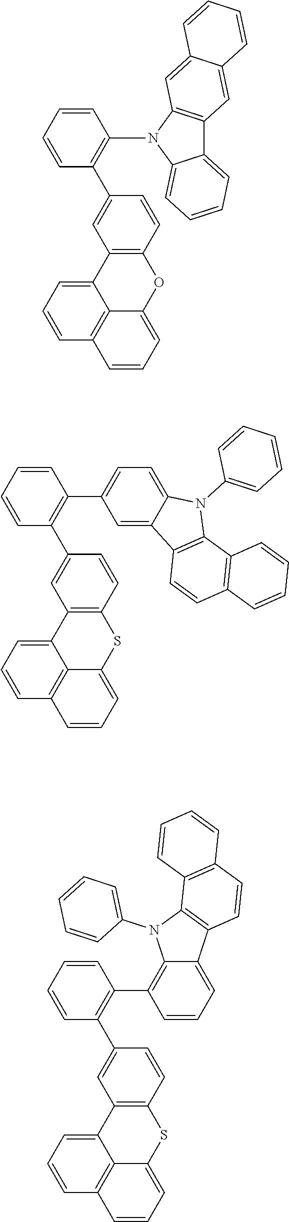 [SMILES: c1ccc(-n2c3ccccc3c3cc4ccccc4cc32)c(-c2ccc3c(c2)-c2cccc4cccc(c24)O3)c1 ; c1ccc(-n2c3ccc(-c4ccccc4-c4ccc5c(c4)-c4cccc6cccc(c46)S5)cc3c3ccc4ccccc4c32)cc1 ; c1ccc(-n2c3c(-c4ccccc4-c4ccc5c(c4)-c4cccc6cccc(c46)S5)cccc3c3ccc4ccccc4c32)cc1]

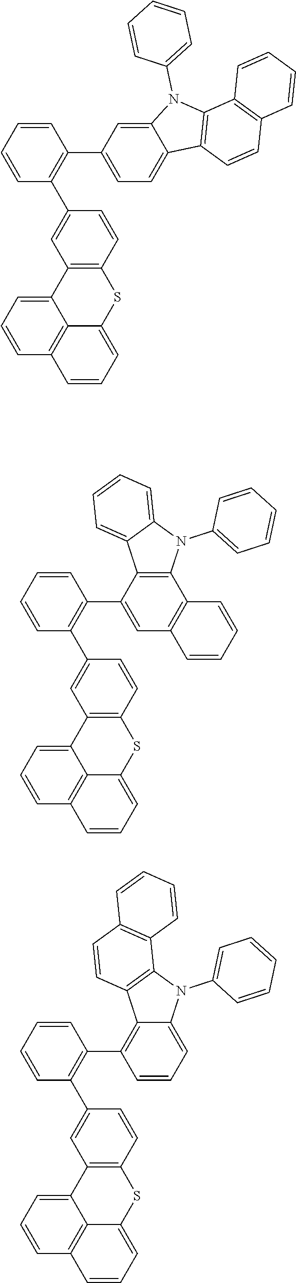

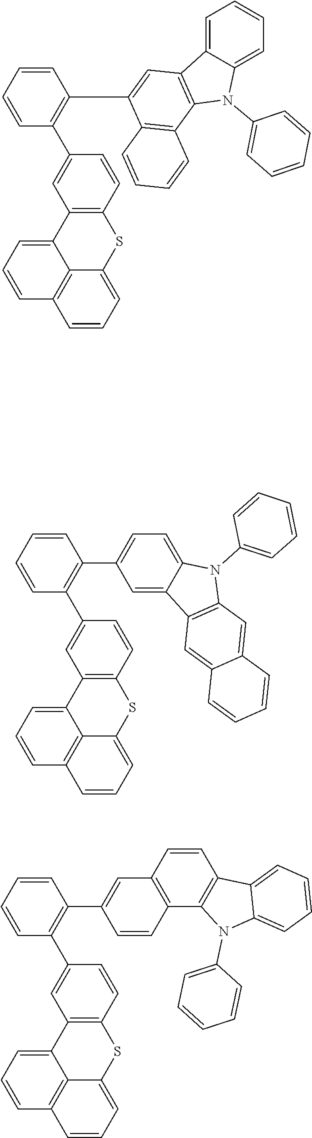 [SMILES: c1ccc(-n2c3ccccc3c3cc(-c4ccccc4-c4ccc5c(c4)-c4cccc6cccc(c46)S5)c4ccccc4c32)cc1 ; c1ccc(-n2c3ccc(-c4ccccc4-c4ccc5c(c4)-c4cccc6cccc(c46)S5)cc3c3cc4ccccc4cc32)cc1 ; c1ccc(-n2c3ccccc3c3ccc4cc(-c5ccccc5-c5ccc6c(c5)-c5cccc7cccc(c57)S6)ccc4c32)cc1]

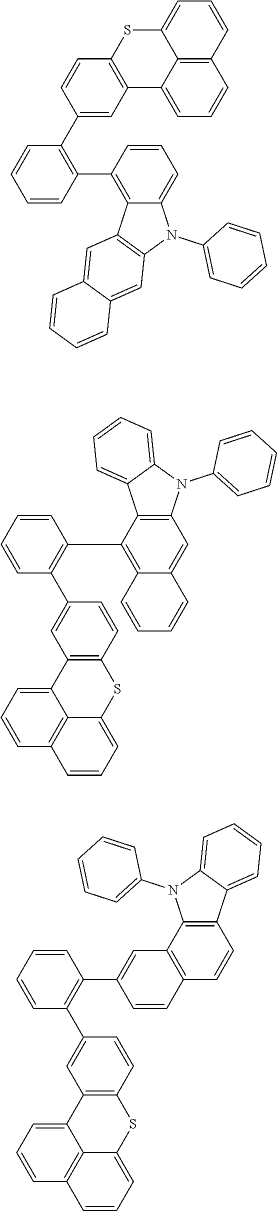 [SMILES: c1ccc(-n2c3cc4ccccc4cc3c3c(-c4ccccc4-c4ccc5c(c4)-c4cccc6cccc(c46)S5)cccc32)cc1 ; c1ccc(-n2c3ccccc3c3c(-c4ccccc4-c4ccc5c(c4)-c4cccc6cccc(c46)S5)c4ccccc4cc32)cc1 ; c1ccc(-n2c3ccccc3c3ccc4ccc(-c5ccccc5-c5ccc6c(c5)-c5cccc7cccc(c57)S6)cc4c32)cc1]

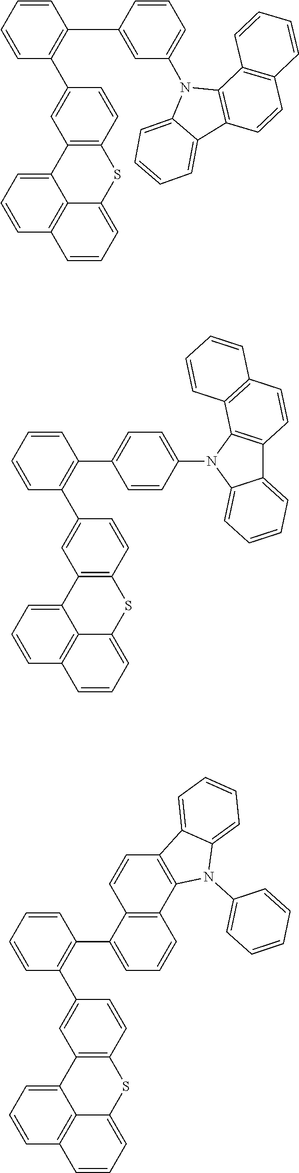 [SMILES: c1cc(-c2ccccc2-c2ccc3c(c2)-c2cccc4cccc(c24)S3)cc(-n2c3ccccc3c3ccc4ccccc4c32)c1 ; c1ccc(-c2ccc3c(c2)-c2cccc4cccc(c24)S3)c(-c2ccc(-n3c4ccccc4c4ccc5ccccc5c43)cc2)c1 ; c1ccc(-n2c3ccccc3c3ccc4c(-c5ccccc5-c5ccc6c(c5)-c5cccc7cccc(c57)S6)cccc4c32)cc1]

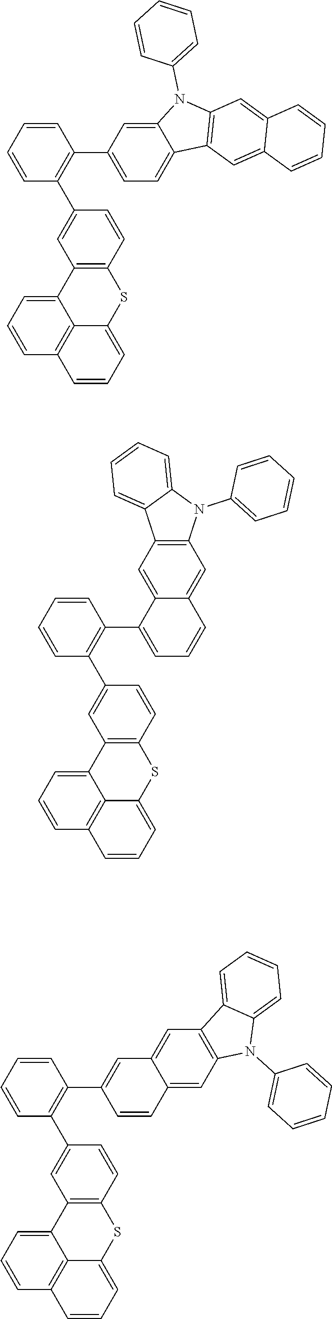 [SMILES: c1ccc(-n2c3cc(-c4ccccc4-c4ccc5c(c4)-c4cccc6cccc(c46)S5)ccc3c3cc4ccccc4cc32)cc1 ; c1ccc(-n2c3ccccc3c3cc4c(-c5ccccc5-c5ccc6c(c5)-c5cccc7cccc(c57)S6)cccc4cc32)cc1 ; c1ccc(-n2c3ccccc3c3cc4cc(-c5ccccc5-c5ccc6c(c5)-c5cccc7cccc(c57)S6)ccc4cc32)cc1]

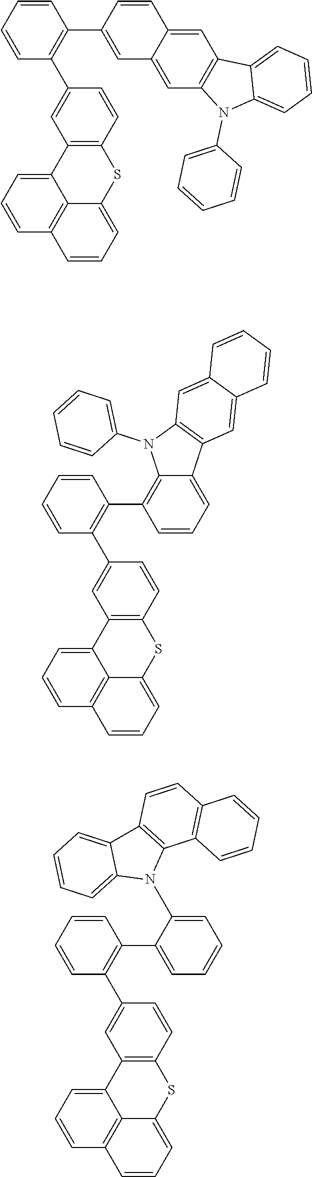 [SMILES: c1ccc(-n2c3ccccc3c3cc4ccc(-c5ccccc5-c5ccc6c(c5)-c5cccc7cccc(c57)S6)cc4cc32)cc1 ; c1ccc(-n2c3cc4ccccc4cc3c3cccc(-c4ccccc4-c4ccc5c(c4)-c4cccc6cccc(c46)S5)c32)cc1 ; c1ccc(-c2ccccc2-n2c3ccccc3c3ccc4ccccc4c32)c(-c2ccc3c(c2)-c2cccc4cccc(c24)S3)c1]

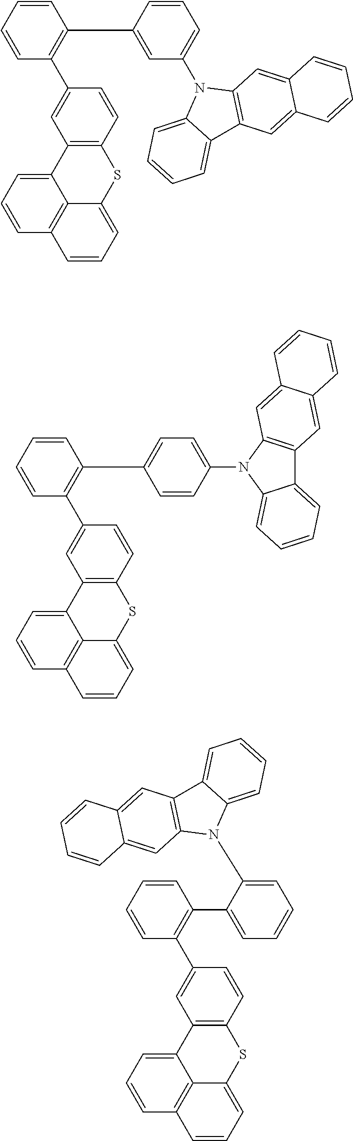 [SMILES: c1cc(-c2ccccc2-c2ccc3c(c2)-c2cccc4cccc(c24)S3)cc(-n2c3ccccc3c3cc4ccccc4cc32)c1 ; c1ccc(-c2ccc3c(c2)-c2cccc4cccc(c24)S3)c(-c2ccc(-n3c4ccccc4c4cc5ccccc5cc43)cc2)c1 ; c1ccc(-c2ccccc2-n2c3ccccc3c3cc4ccccc4cc32)c(-c2ccc3c(c2)-c2cccc4cccc(c24)S3)c1]

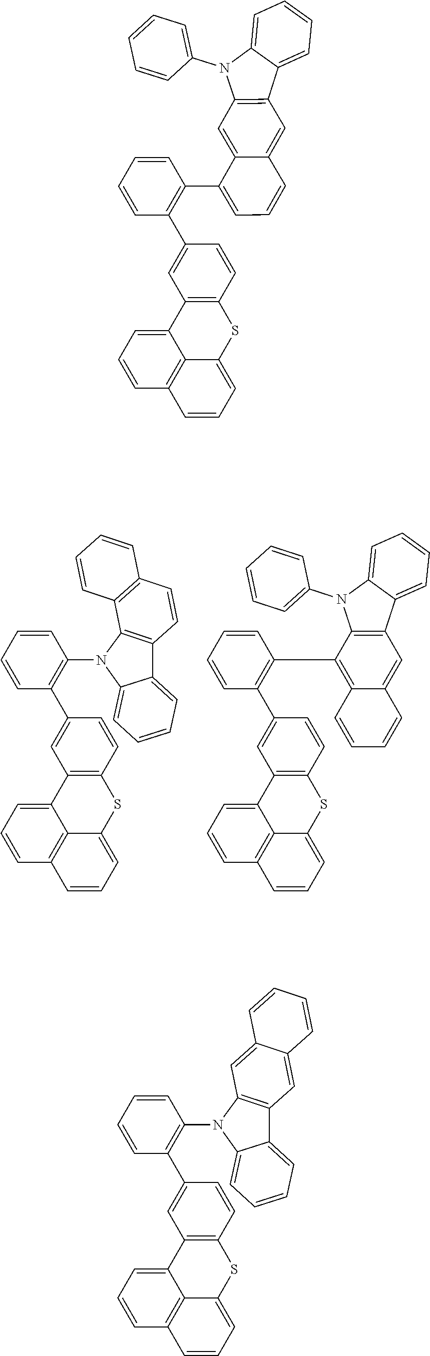 [SMILES: c1ccc(-n2c3ccccc3c3cc4cccc(-c5ccccc5-c5ccc6c(c5)-c5cccc7cccc(c57)S6)c4cc32)cc1 ; c1ccc(-n2c3ccccc3c3ccc4ccccc4c32)c(-c2ccc3c(c2)-c2cccc4cccc(c24)S3)c1 ; c1ccc(-n2c3ccccc3c3cc4ccccc4c(-c4ccccc4-c4ccc5c(c4)-c4cccc6cccc(c46)S5)c32)cc1 ; c1ccc(-n2c3ccccc3c3cc4ccccc4cc32)c(-c2ccc3c(c2)-c2cccc4cccc(c24)S3)c1]

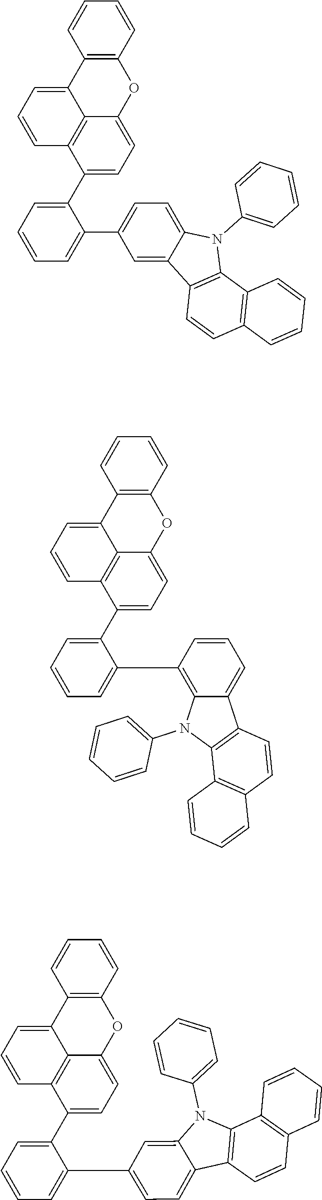 [SMILES: c1ccc(-n2c3ccc(-c4ccccc4-c4ccc5c6c(cccc46)-c4ccccc4O5)cc3c3ccc4ccccc4c32)cc1 ; c1ccc(-n2c3c(-c4ccccc4-c4ccc5c6c(cccc46)-c4ccccc4O5)cccc3c3ccc4ccccc4c32)cc1 ; c1ccc(-n2c3cc(-c4ccccc4-c4ccc5c6c(cccc46)-c4ccccc4O5)ccc3c3ccc4ccccc4c32)cc1]

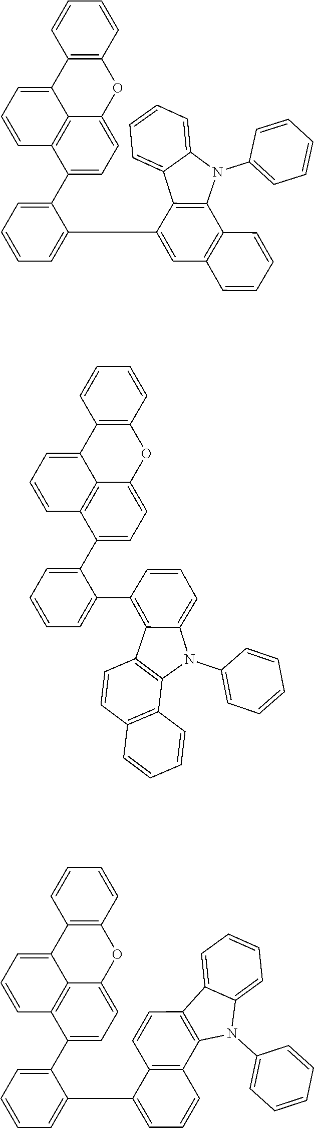 [SMILES: c1ccc(-n2c3ccccc3c3c(-c4ccccc4-c4ccc5c6c(cccc46)-c4ccccc4O5)cc4ccccc4c32)cc1 ; c1ccc(-n2c3cccc(-c4ccccc4-c4ccc5c6c(cccc46)-c4ccccc4O5)c3c3ccc4ccccc4c32)cc1 ; c1ccc(-n2c3ccccc3c3ccc4c(-c5ccccc5-c5ccc6c7c(cccc57)-c5ccccc5O6)cccc4c32)cc1]

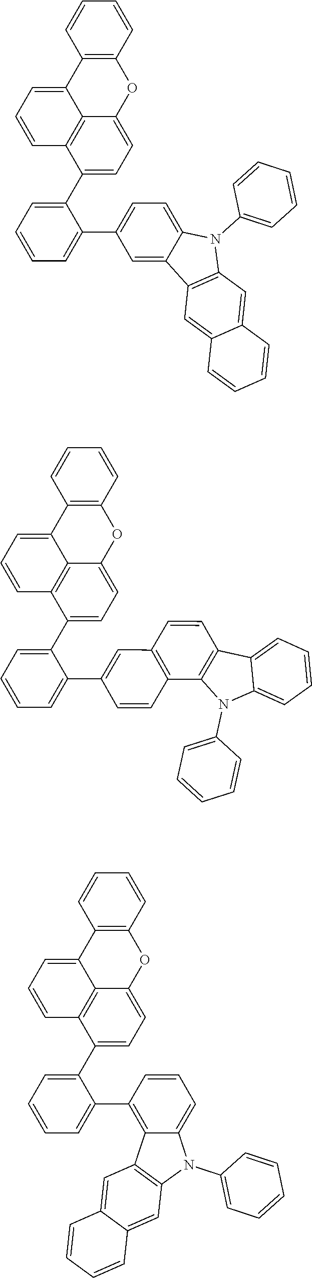 [SMILES: c1ccc(-n2c3ccc(-c4ccccc4-c4ccc5c6c(cccc46)-c4ccccc4O5)cc3c3cc4ccccc4cc32)cc1 ; c1ccc(-n2c3ccccc3c3ccc4cc(-c5ccccc5-c5ccc6c7c(cccc57)-c5ccccc5O6)ccc4c32)cc1 ; c1ccc(-n2c3cc4ccccc4cc3c3c(-c4ccccc4-c4ccc5c6c(cccc46)-c4ccccc4O5)cccc32)cc1]

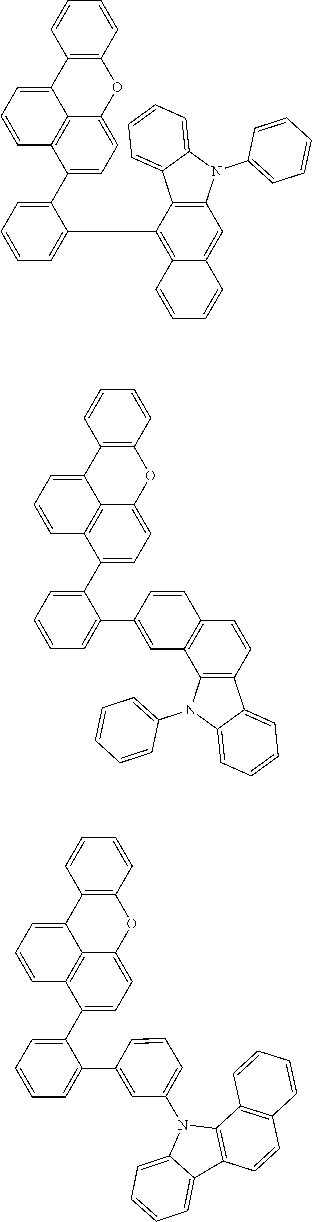 [SMILES: c1ccc(-n2c3ccccc3c3c(-c4ccccc4-c4ccc5c6c(cccc46)-c4ccccc4O5)c4ccccc4cc32)cc1 ; c1ccc(-n2c3ccccc3c3ccc4ccc(-c5ccccc5-c5ccc6c7c(cccc57)-c5ccccc5O6)cc4c32)cc1 ; c1cc(-c2ccccc2-c2ccc3c4c(cccc24)-c2ccccc2O3)cc(-n2c3ccccc3c3ccc4ccccc4c32)c1]

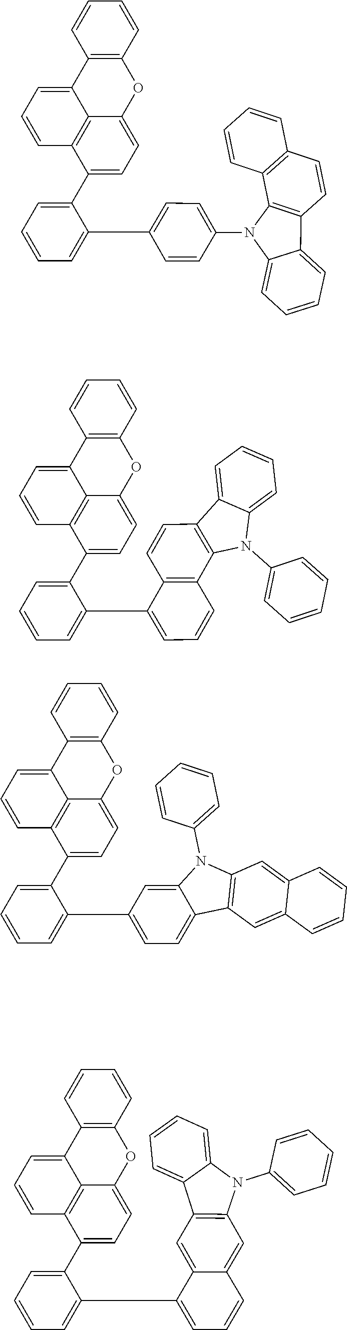

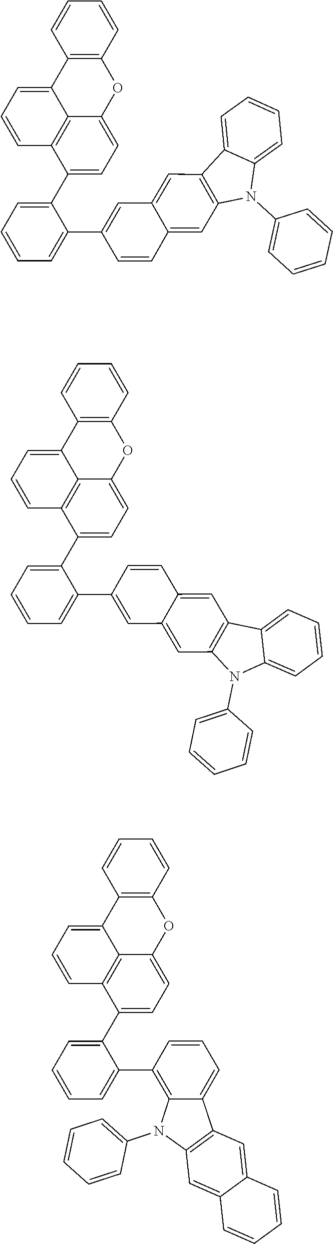 [SMILES: c1ccc(-n2c3ccccc3c3cc4cc(-c5ccccc5-c5ccc6c7c(cccc57)-c5ccccc5O6)ccc4cc32)cc1 ; c1ccc(-n2c3ccccc3c3cc4ccc(-c5ccccc5-c5ccc6c7c(cccc57)-c5ccccc5O6)cc4cc32)cc1 ; c1ccc(-n2c3cc4ccccc4cc3c3cccc(-c4ccccc4-c4ccc5c6c(cccc46)-c4ccccc4O5)c32)cc1]

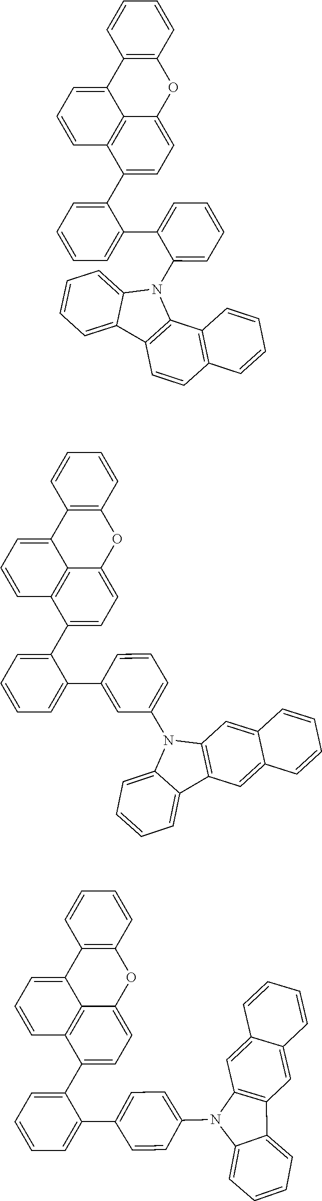 [SMILES: c1ccc2c(c1)Oc1ccc(-c3ccccc3-c3ccccc3-n3c4ccccc4c4ccc5ccccc5c43)c3cccc-2c13 ; c1cc(-c2ccccc2-c2ccc3c4c(cccc24)-c2ccccc2O3)cc(-n2c3ccccc3c3cc4ccccc4cc32)c1 ; c1ccc2c(c1)Oc1ccc(-c3ccccc3-c3ccc(-n4c5ccccc5c5cc6ccccc6cc54)cc3)c3cccc-2c13]

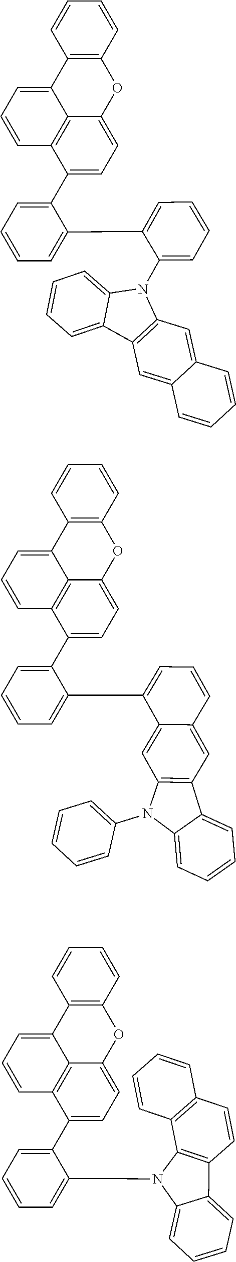 [SMILES: c1ccc2c(c1)Oc1ccc(-c3ccccc3-c3ccccc3-n3c4ccccc4c4cc5ccccc5cc43)c3cccc-2c13 ; c1ccc(-n2c3ccccc3c3cc4cccc(-c5ccccc5-c5ccc6c7c(cccc57)-c5ccccc5O6)c4cc32)cc1 ; c1ccc2c(c1)Oc1ccc(-c3ccccc3-n3c4ccccc4c4ccc5ccccc5c43)c3cccc-2c13]

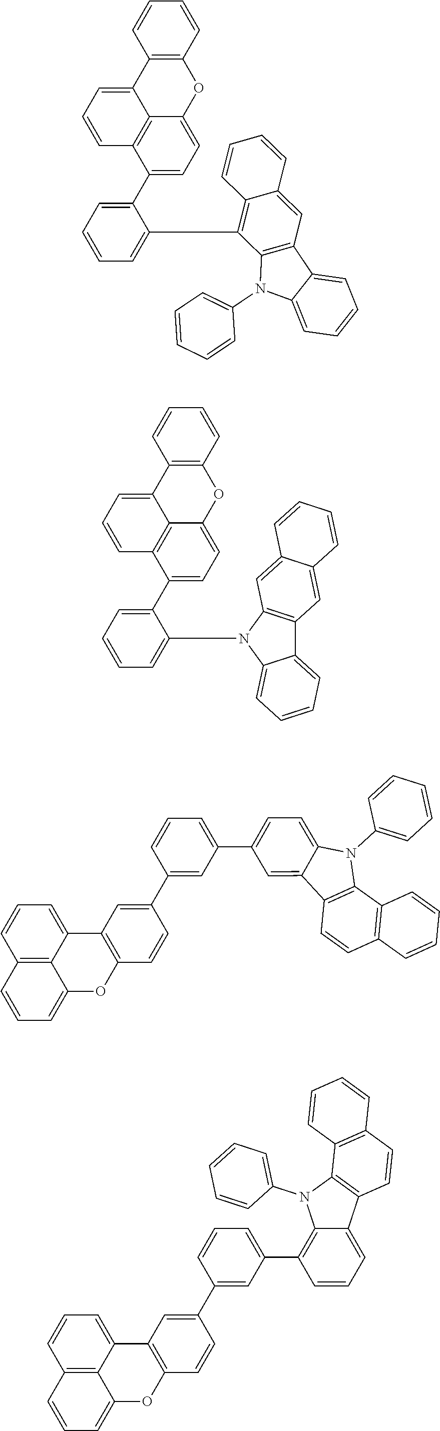 [SMILES: c1ccc(-n2c3ccccc3c3cc4ccccc4c(-c4ccccc4-c4ccc5c6c(cccc46)-c4ccccc4O5)c32)cc1 ; c1ccc2c(c1)Oc1ccc(-c3ccccc3-n3c4ccccc4c4cc5ccccc5cc43)c3cccc-2c13 ; c1ccc(-n2c3ccc(-c4cccc(-c5ccc6c(c5)-c5cccc7cccc(c57)O6)c4)cc3c3ccc4ccccc4c32)cc1 ; c1ccc(-n2c3c(-c4cccc(-c5ccc6c(c5)-c5cccc7cccc(c57)O6)c4)cccc3c3ccc4ccccc4c32)cc1]

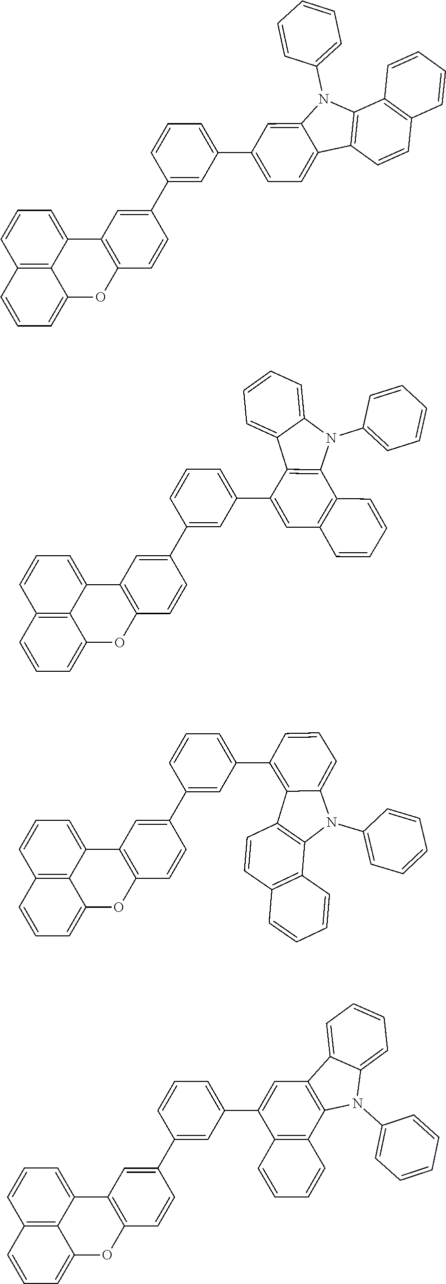 [SMILES: c1ccc(-n2c3cc(-c4cccc(-c5ccc6c(c5)-c5cccc7cccc(c57)O6)c4)ccc3c3ccc4ccccc4c32)cc1 ; c1ccc(-n2c3ccccc3c3c(-c4cccc(-c5ccc6c(c5)-c5cccc7cccc(c57)O6)c4)cc4ccccc4c32)cc1 ; c1ccc(-n2c3cccc(-c4cccc(-c5ccc6c(c5)-c5cccc7cccc(c57)O6)c4)c3c3ccc4ccccc4c32)cc1 ; c1ccc(-n2c3ccccc3c3cc(-c4cccc(-c5ccc6c(c5)-c5cccc7cccc(c57)O6)c4)c4ccccc4c32)cc1]

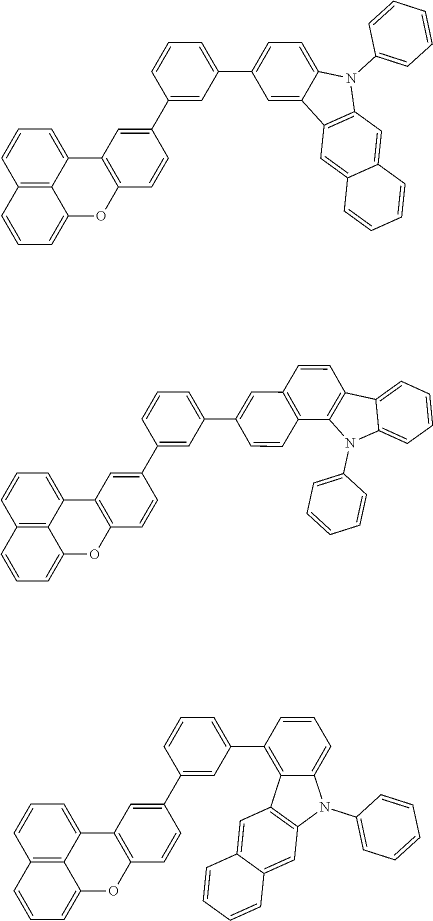 [SMILES: c1ccc(-n2c3ccc(-c4cccc(-c5ccc6c(c5)-c5cccc7cccc(c57)O6)c4)cc3c3cc4ccccc4cc32)cc1 ; c1ccc(-n2c3ccccc3c3ccc4cc(-c5cccc(-c6ccc7c(c6)-c6cccc8cccc(c68)O7)c5)ccc4c32)cc1 ; c1ccc(-n2c3cc4ccccc4cc3c3c(-c4cccc(-c5ccc6c(c5)-c5cccc7cccc(c57)O6)c4)cccc32)cc1]

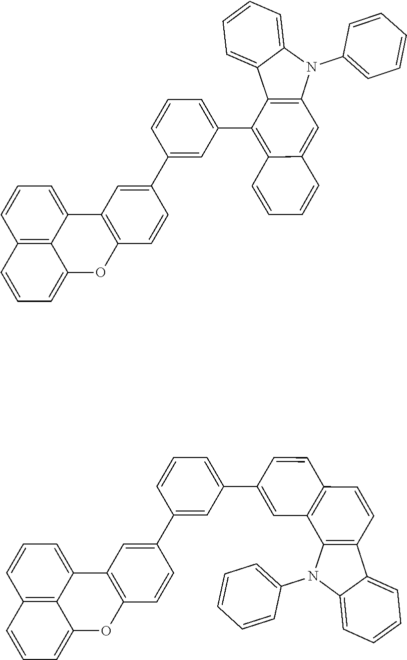 [SMILES: c1ccc(-n2c3ccccc3c3c(-c4cccc(-c5ccc6c(c5)-c5cccc7cccc(c57)O6)c4)c4ccccc4cc32)cc1 ; c1ccc(-n2c3ccccc3c3ccc4ccc(-c5cccc(-c6ccc7c(c6)-c6cccc8cccc(c68)O7)c5)cc4c32)cc1]

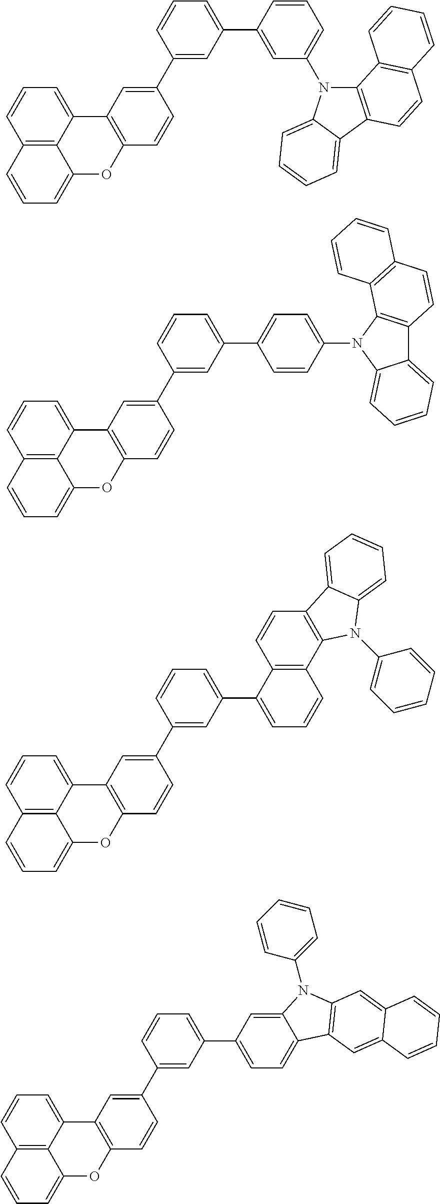 [SMILES: c1cc(-c2cccc(-n3c4ccccc4c4ccc5ccccc5c43)c2)cc(-c2ccc3c(c2)-c2cccc4cccc(c24)O3)c1 ; c1cc(-c2ccc(-n3c4ccccc4c4ccc5ccccc5c43)cc2)cc(-c2ccc3c(c2)-c2cccc4cccc(c24)O3)c1 ; c1ccc(-n2c3ccccc3c3ccc4c(-c5cccc(-c6ccc7c(c6)-c6cccc8cccc(c68)O7)c5)cccc4c32)cc1 ; c1ccc(-n2c3cc(-c4cccc(-c5ccc6c(c5)-c5cccc7cccc(c57)O6)c4)ccc3c3cc4ccccc4cc32)cc1]

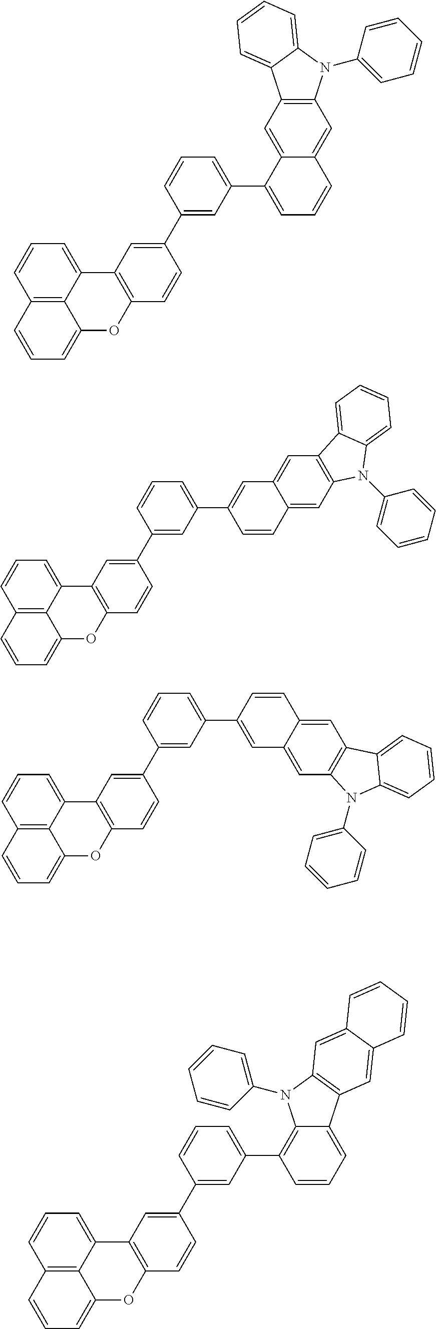 [SMILES: c1ccc(-n2c3ccccc3c3cc4c(-c5cccc(-c6ccc7c(c6)-c6cccc8cccc(c68)O7)c5)cccc4cc32)cc1 ; c1ccc(-n2c3ccccc3c3cc4cc(-c5cccc(-c6ccc7c(c6)-c6cccc8cccc(c68)O7)c5)ccc4cc32)cc1 ; c1ccc(-n2c3ccccc3c3cc4ccc(-c5cccc(-c6ccc7c(c6)-c6cccc8cccc(c68)O7)c5)cc4cc32)cc1 ; c1ccc(-n2c3cc4ccccc4cc3c3cccc(-c4cccc(-c5ccc6c(c5)-c5cccc7cccc(c57)O6)c4)c32)cc1]

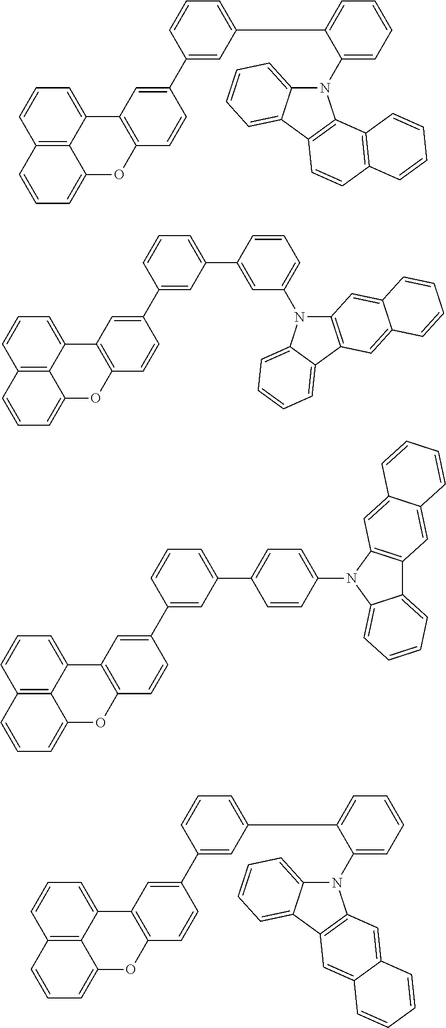 [SMILES: c1cc(-c2ccc3c(c2)-c2cccc4cccc(c24)O3)cc(-c2ccccc2-n2c3ccccc3c3ccc4ccccc4c32)c1 ; c1cc(-c2cccc(-n3c4ccccc4c4cc5ccccc5cc43)c2)cc(-c2ccc3c(c2)-c2cccc4cccc(c24)O3)c1 ; c1cc(-c2ccc(-n3c4ccccc4c4cc5ccccc5cc43)cc2)cc(-c2ccc3c(c2)-c2cccc4cccc(c24)O3)c1 ; c1cc(-c2ccc3c(c2)-c2cccc4cccc(c24)O3)cc(-c2ccccc2-n2c3ccccc3c3cc4ccccc4cc32)c1]

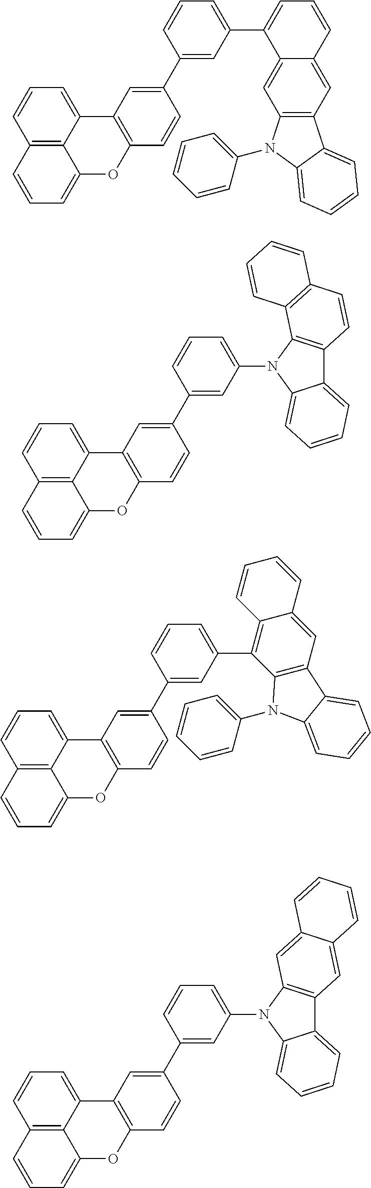

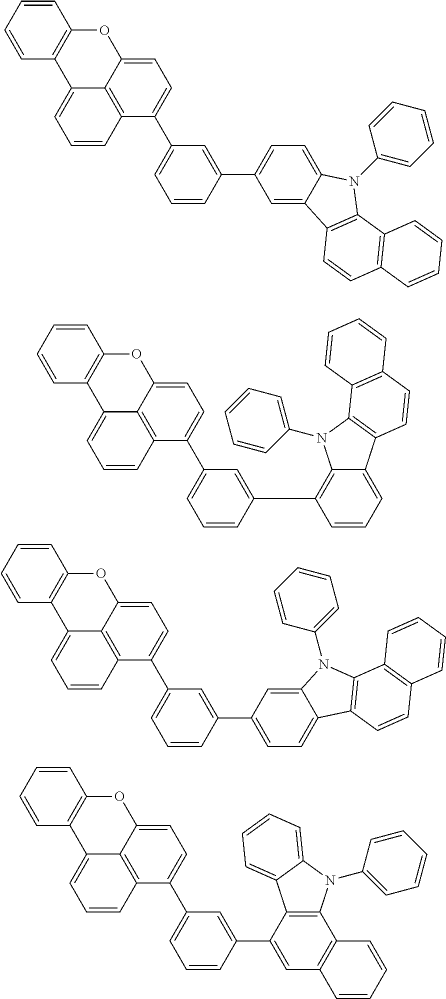

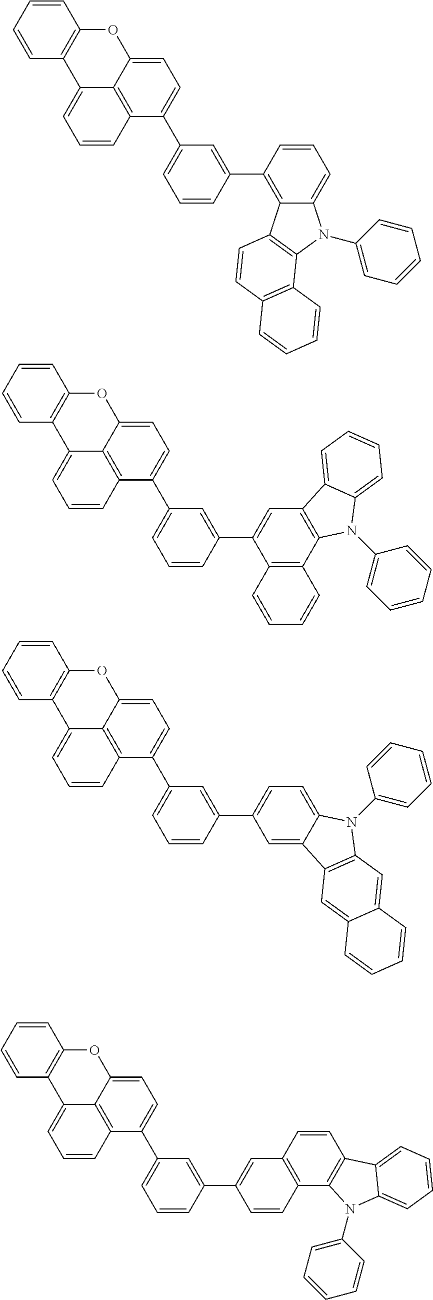 [SMILES: c1ccc(-n2c3cccc(-c4cccc(-c5ccc6c7c(cccc57)-c5ccccc5O6)c4)c3c3ccc4ccccc4c32)cc1 ; c1ccc(-n2c3ccccc3c3cc(-c4cccc(-c5ccc6c7c(cccc57)-c5ccccc5O6)c4)c4ccccc4c32)cc1 ; c1ccc(-n2c3ccc(-c4cccc(-c5ccc6c7c(cccc57)-c5ccccc5O6)c4)cc3c3cc4ccccc4cc32)cc1 ; c1ccc(-n2c3ccccc3c3ccc4cc(-c5cccc(-c6ccc7c8c(cccc68)-c6ccccc6O7)c5)ccc4c32)cc1]

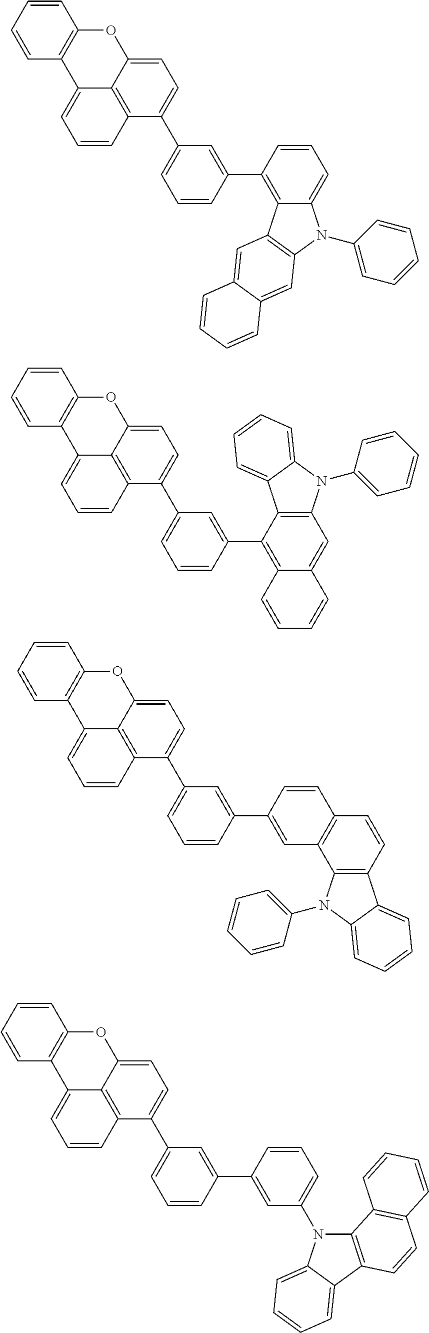 [SMILES: c1ccc(-n2c3cc4ccccc4cc3c3c(-c4cccc(-c5ccc6c7c(cccc57)-c5ccccc5O6)c4)cccc32)cc1 ; c1ccc(-n2c3ccccc3c3c(-c4cccc(-c5ccc6c7c(cccc57)-c5ccccc5O6)c4)c4ccccc4cc32)cc1 ; c1ccc(-n2c3ccccc3c3ccc4ccc(-c5cccc(-c6ccc7c8c(cccc68)-c6ccccc6O7)c5)cc4c32)cc1 ; c1cc(-c2cccc(-n3c4ccccc4c4ccc5ccccc5c43)c2)cc(-c2ccc3c4c(cccc24)-c2ccccc2O3)c1]

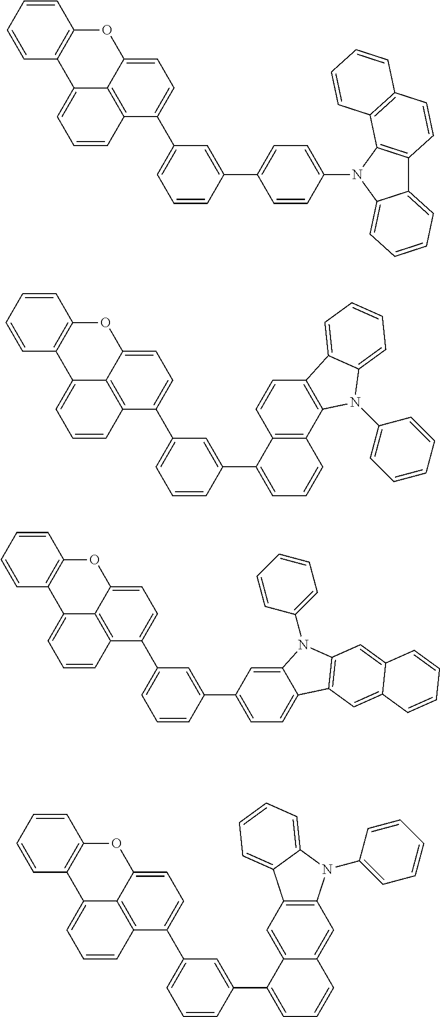

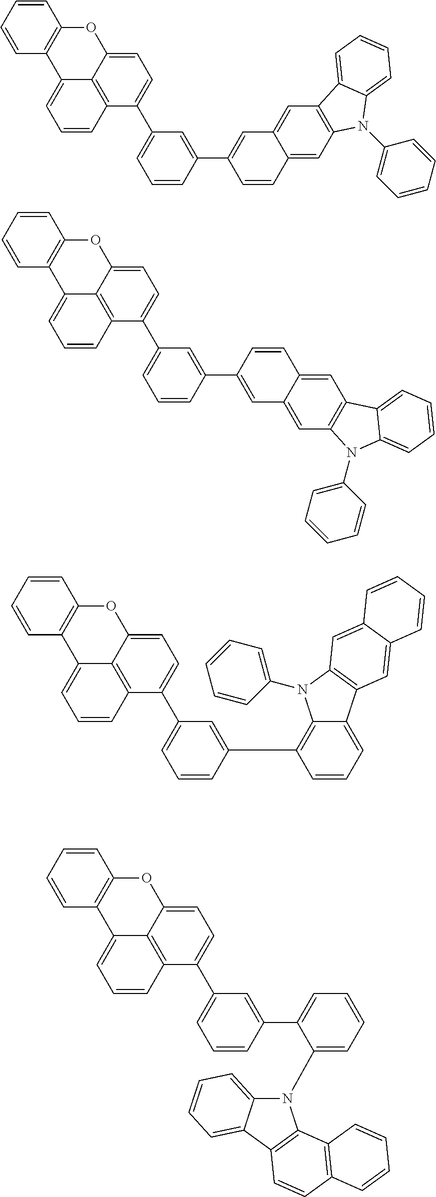 [SMILES: c1ccc(-n2c3ccccc3c3cc4cc(-c5cccc(-c6ccc7c8c(cccc68)-c6ccccc6O7)c5)ccc4cc32)cc1 ; c1ccc(-n2c3ccccc3c3cc4ccc(-c5cccc(-c6ccc7c8c(cccc68)-c6ccccc6O7)c5)cc4cc32)cc1 ; c1ccc(-n2c3cc4ccccc4cc3c3cccc(-c4cccc(-c5ccc6c7c(cccc57)-c5ccccc5O6)c4)c32)cc1 ; c1cc(-c2ccccc2-n2c3ccccc3c3ccc4ccccc4c32)cc(-c2ccc3c4c(cccc24)-c2ccccc2O3)c1]

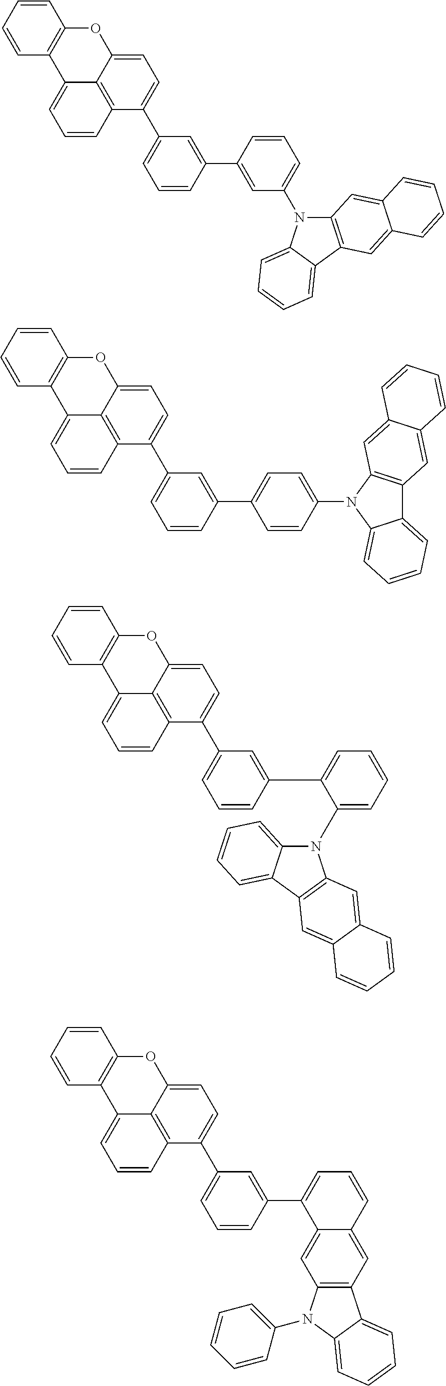 [SMILES: c1cc(-c2cccc(-n3c4ccccc4c4cc5ccccc5cc43)c2)cc(-c2ccc3c4c(cccc24)-c2ccccc2O3)c1 ; c1cc(-c2ccc(-n3c4ccccc4c4cc5ccccc5cc43)cc2)cc(-c2ccc3c4c(cccc24)-c2ccccc2O3)c1 ; c1cc(-c2ccccc2-n2c3ccccc3c3cc4ccccc4cc32)cc(-c2ccc3c4c(cccc24)-c2ccccc2O3)c1 ; c1ccc(-n2c3ccccc3c3cc4cccc(-c5cccc(-c6ccc7c8c(cccc68)-c6ccccc6O7)c5)c4cc32)cc1]

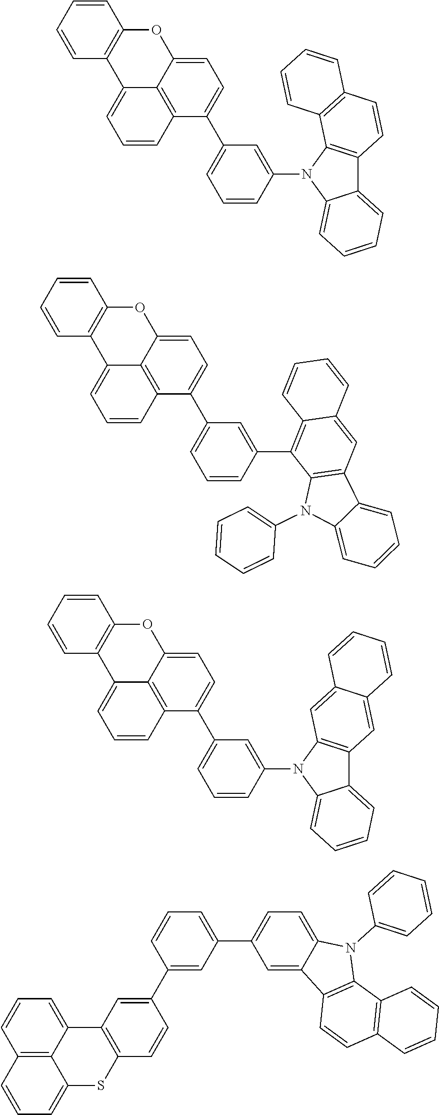

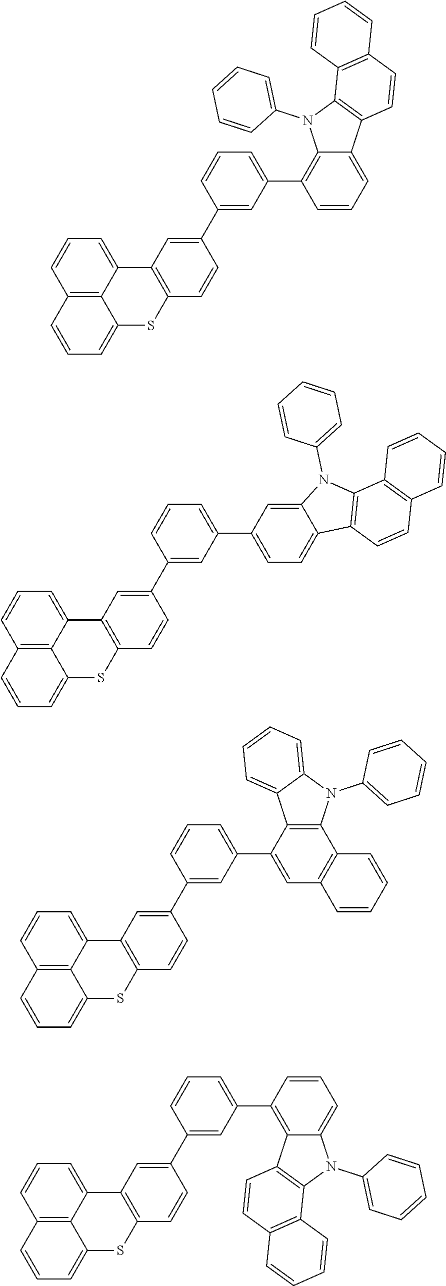 [SMILES: c1ccc(-n2c3c(-c4cccc(-c5ccc6c(c5)-c5cccc7cccc(c57)S6)c4)cccc3c3ccc4ccccc4c32)cc1 ; c1ccc(-n2c3cc(-c4cccc(-c5ccc6c(c5)-c5cccc7cccc(c57)S6)c4)ccc3c3ccc4ccccc4c32)cc1 ; c1ccc(-n2c3ccccc3c3c(-c4cccc(-c5ccc6c(c5)-c5cccc7cccc(c57)S6)c4)cc4ccccc4c32)cc1 ; c1ccc(-n2c3cccc(-c4cccc(-c5ccc6c(c5)-c5cccc7cccc(c57)S6)c4)c3c3ccc4ccccc4c32)cc1]

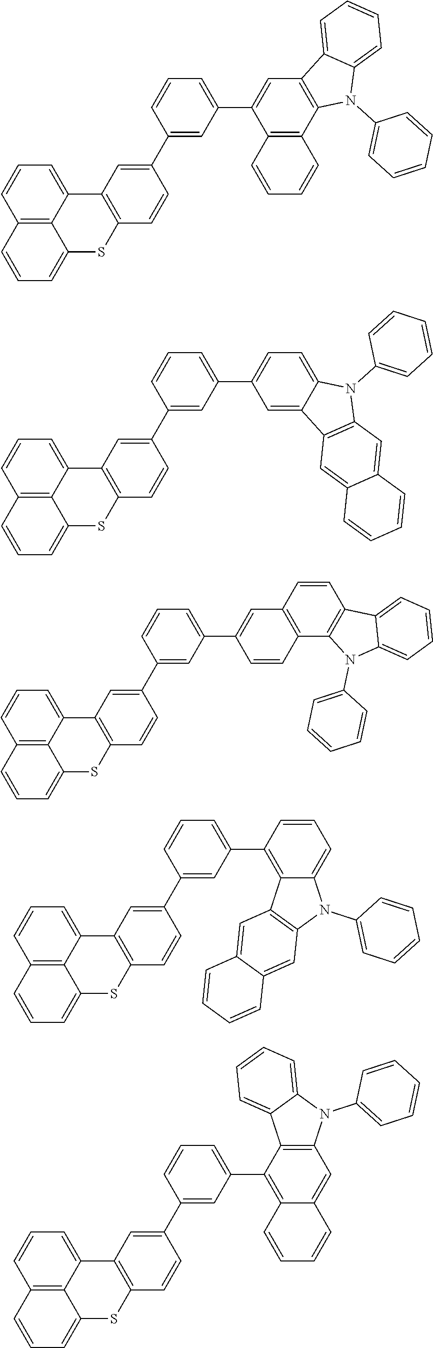

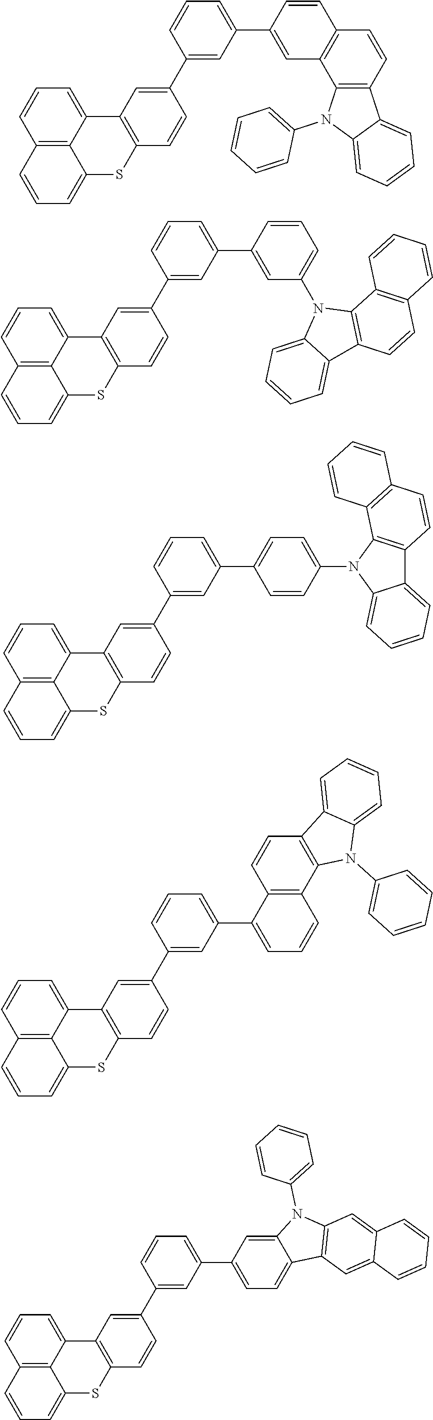 [SMILES: c1ccc(-n2c3ccccc3c3ccc4ccc(-c5cccc(-c6ccc7c(c6)-c6cccc8cccc(c68)S7)c5)cc4c32)cc1 ; c1cc(-c2cccc(-n3c4ccccc4c4ccc5ccccc5c43)c2)cc(-c2ccc3c(c2)-c2cccc4cccc(c24)S3)c1 ; c1cc(-c2ccc(-n3c4ccccc4c4ccc5ccccc5c43)cc2)cc(-c2ccc3c(c2)-c2cccc4cccc(c24)S3)c1 ; c1ccc(-n2c3ccccc3c3ccc4c(-c5cccc(-c6ccc7c(c6)-c6cccc8cccc(c68)S7)c5)cccc4c32)cc1 ; c1ccc(-n2c3cc(-c4cccc(-c5ccc6c(c5)-c5cccc7cccc(c57)S6)c4)ccc3c3cc4ccccc4cc32)cc1]

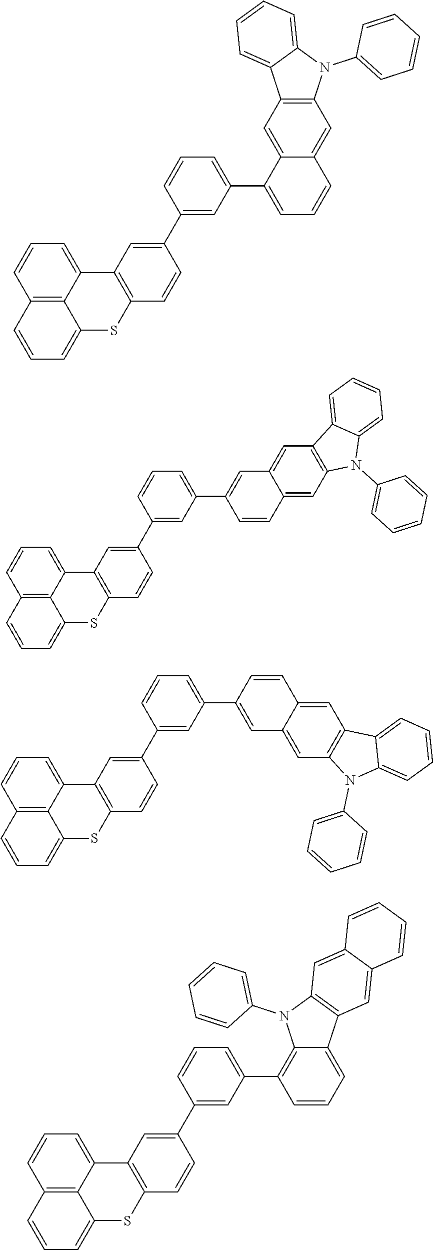

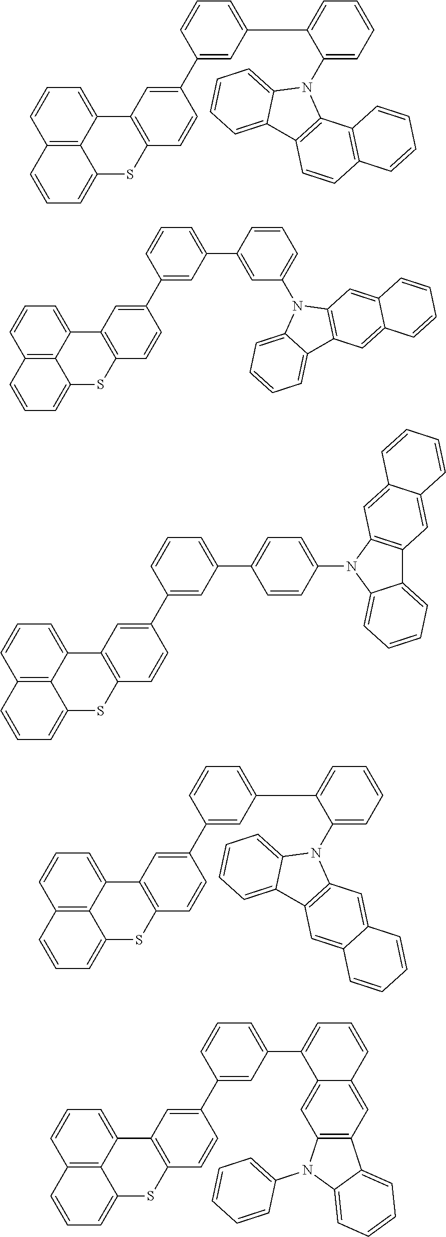

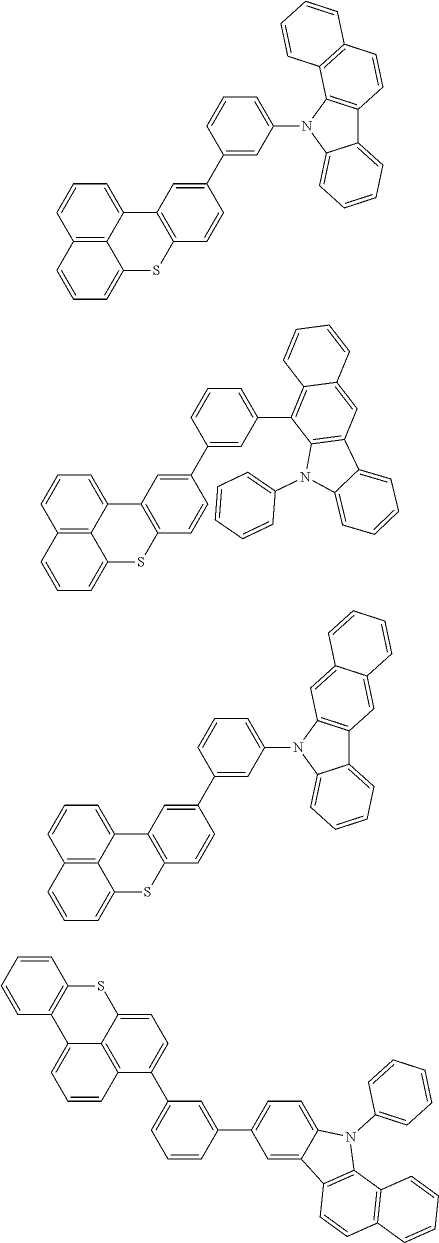

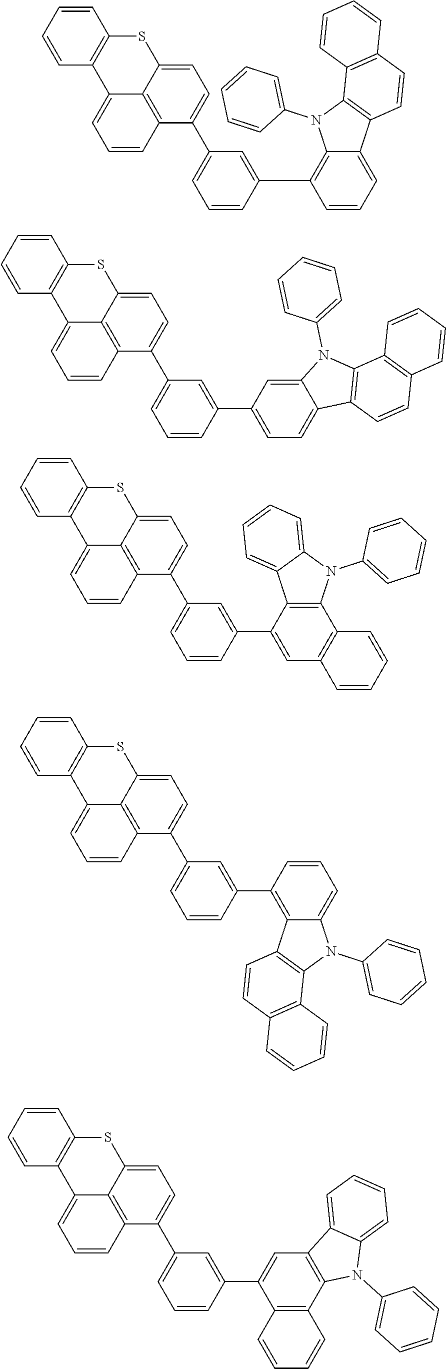 [SMILES: c1ccc(-n2c3c(-c4cccc(-c5ccc6c7c(cccc57)-c5ccccc5S6)c4)cccc3c3ccc4ccccc4c32)cc1 ; c1ccc(-n2c3cc(-c4cccc(-c5ccc6c7c(cccc57)-c5ccccc5S6)c4)ccc3c3ccc4ccccc4c32)cc1 ; c1ccc(-n2c3ccccc3c3c(-c4cccc(-c5ccc6c7c(cccc57)-c5ccccc5S6)c4)cc4ccccc4c32)cc1 ; c1ccc(-n2c3cccc(-c4cccc(-c5ccc6c7c(cccc57)-c5ccccc5S6)c4)c3c3ccc4ccccc4c32)cc1 ; c1ccc(-n2c3ccccc3c3cc(-c4cccc(-c5ccc6c7c(cccc57)-c5ccccc5S6)c4)c4ccccc4c32)cc1]

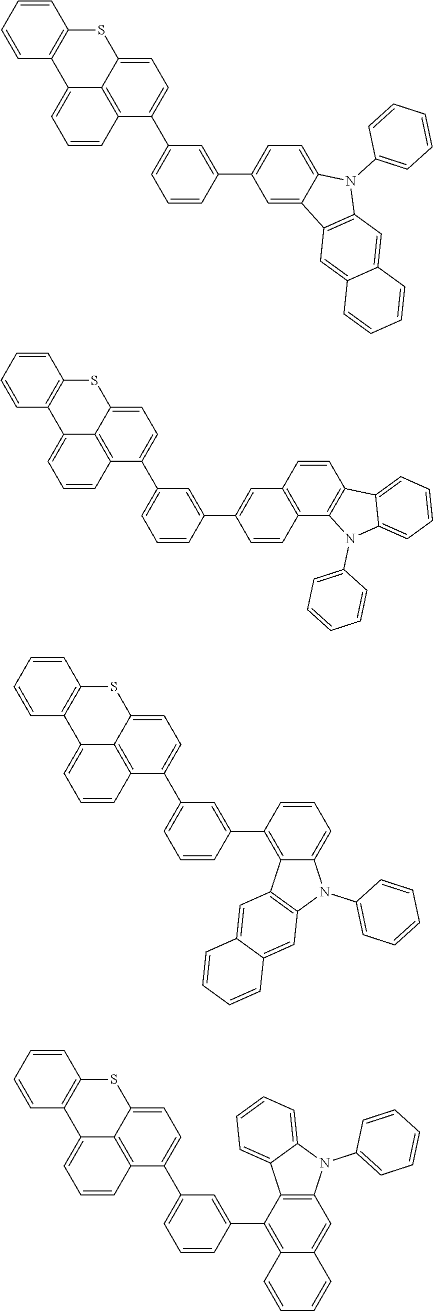 [SMILES: c1ccc(-n2c3ccc(-c4cccc(-c5ccc6c7c(cccc57)-c5ccccc5S6)c4)cc3c3cc4ccccc4cc32)cc1 ; c1ccc(-n2c3ccccc3c3ccc4cc(-c5cccc(-c6ccc7c8c(cccc68)-c6ccccc6S7)c5)ccc4c32)cc1 ; c1ccc(-n2c3cc4ccccc4cc3c3c(-c4cccc(-c5ccc6c7c(cccc57)-c5ccccc5S6)c4)cccc32)cc1 ; c1ccc(-n2c3ccccc3c3c(-c4cccc(-c5ccc6c7c(cccc57)-c5ccccc5S6)c4)c4ccccc4cc32)cc1]

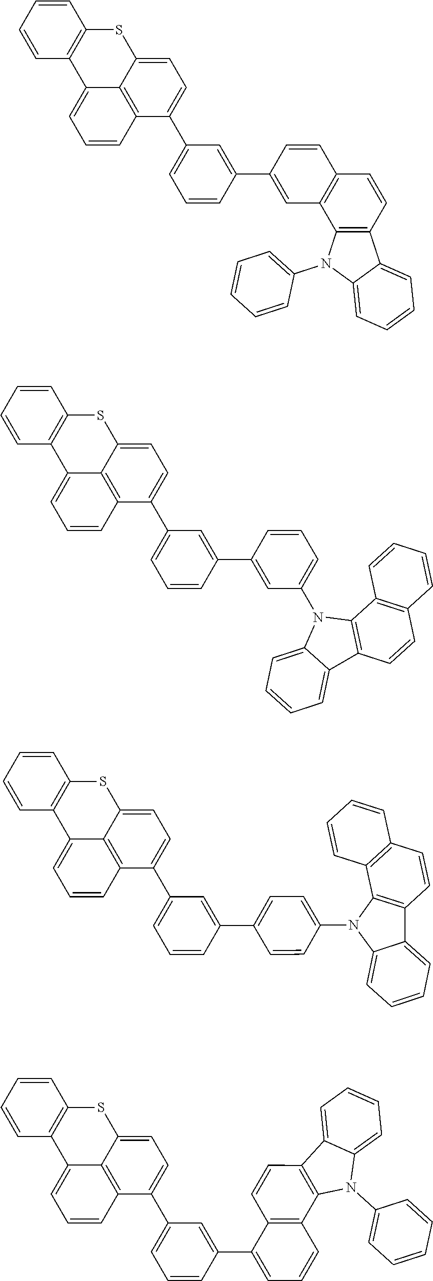 [SMILES: c1ccc(-n2c3ccccc3c3ccc4ccc(-c5cccc(-c6ccc7c8c(cccc68)-c6ccccc6S7)c5)cc4c32)cc1 ; c1cc(-c2cccc(-n3c4ccccc4c4ccc5ccccc5c43)c2)cc(-c2ccc3c4c(cccc24)-c2ccccc2S3)c1 ; c1cc(-c2ccc(-n3c4ccccc4c4ccc5ccccc5c43)cc2)cc(-c2ccc3c4c(cccc24)-c2ccccc2S3)c1 ; c1ccc(-n2c3ccccc3c3ccc4c(-c5cccc(-c6ccc7c8c(cccc68)-c6ccccc6S7)c5)cccc4c32)cc1]

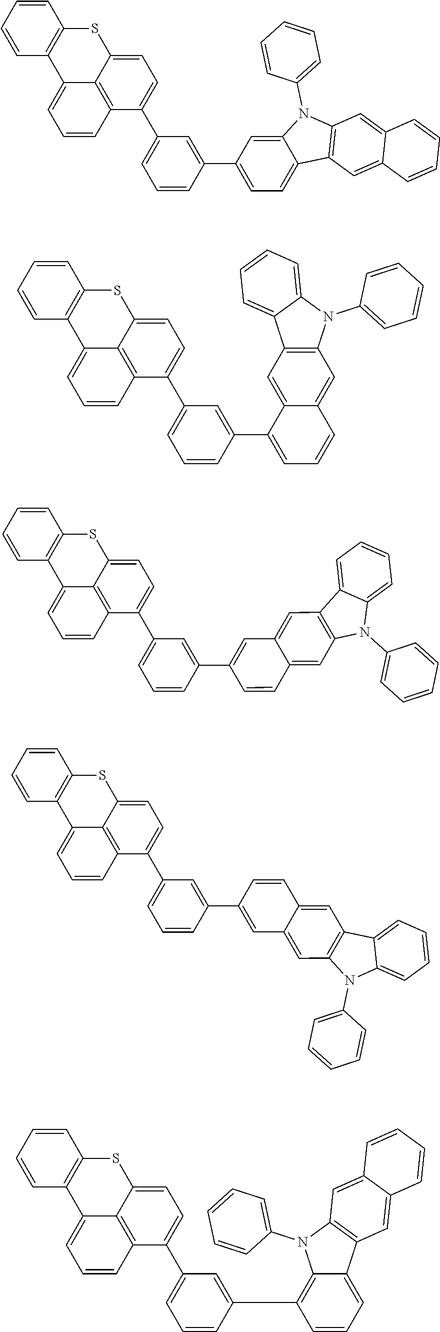 [SMILES: c1ccc(-n2c3cc(-c4cccc(-c5ccc6c7c(cccc57)-c5ccccc5S6)c4)ccc3c3cc4ccccc4cc32)cc1 ; c1ccc(-n2c3ccccc3c3cc4c(-c5cccc(-c6ccc7c8c(cccc68)-c6ccccc6S7)c5)cccc4cc32)cc1 ; c1ccc(-n2c3ccccc3c3cc4cc(-c5cccc(-c6ccc7c8c(cccc68)-c6ccccc6S7)c5)ccc4cc32)cc1 ; c1ccc(-n2c3ccccc3c3cc4ccc(-c5cccc(-c6ccc7c8c(cccc68)-c6ccccc6S7)c5)cc4cc32)cc1 ; c1ccc(-n2c3cc4ccccc4cc3c3cccc(-c4cccc(-c5ccc6c7c(cccc57)-c5ccccc5S6)c4)c32)cc1]

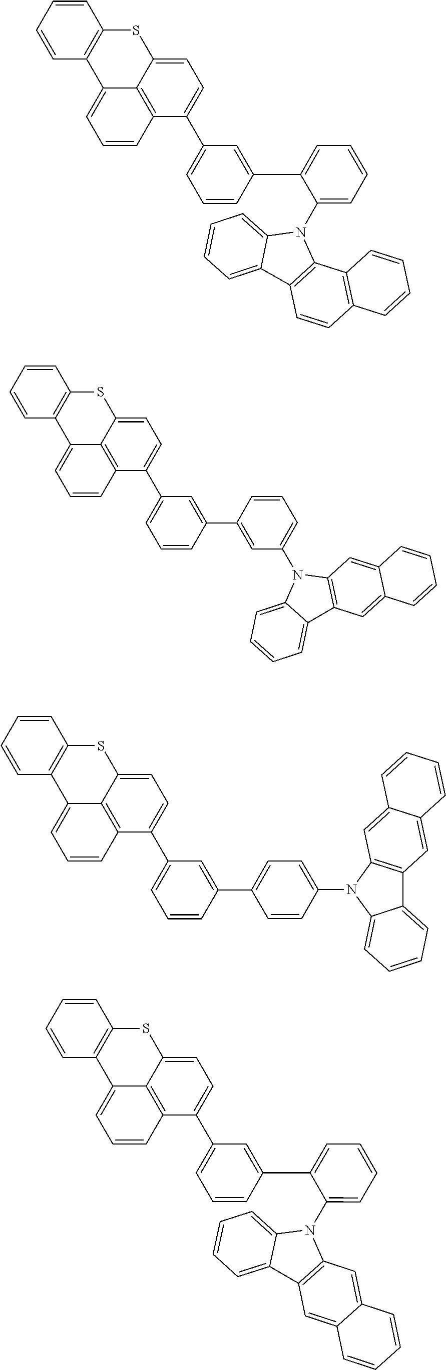 [SMILES: c1cc(-c2ccccc2-n2c3ccccc3c3ccc4ccccc4c32)cc(-c2ccc3c4c(cccc24)-c2ccccc2S3)c1 ; c1cc(-c2cccc(-n3c4ccccc4c4cc5ccccc5cc43)c2)cc(-c2ccc3c4c(cccc24)-c2ccccc2S3)c1 ; c1cc(-c2ccc(-n3c4ccccc4c4cc5ccccc5cc43)cc2)cc(-c2ccc3c4c(cccc24)-c2ccccc2S3)c1 ; c1cc(-c2ccccc2-n2c3ccccc3c3cc4ccccc4cc32)cc(-c2ccc3c4c(cccc24)-c2ccccc2S3)c1]

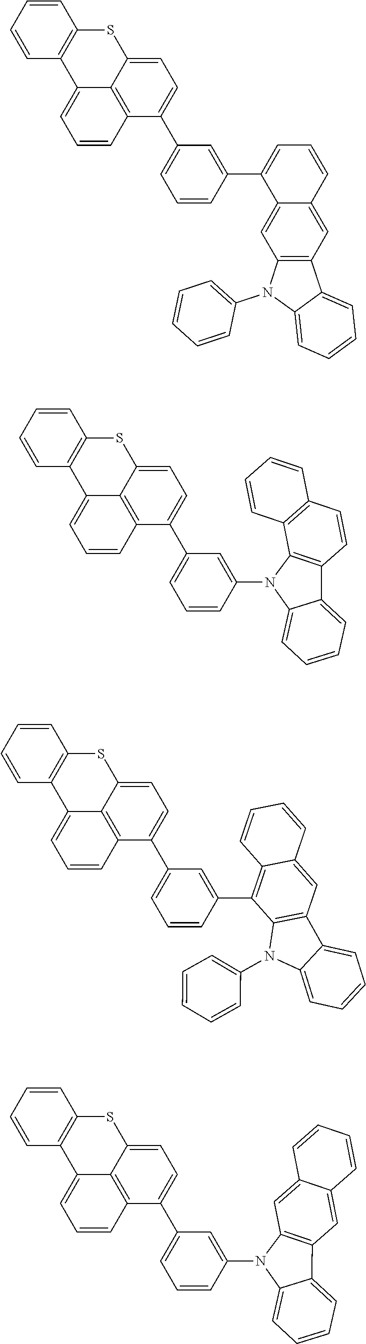 [SMILES: c1ccc(-n2c3ccccc3c3cc4cccc(-c5cccc(-c6ccc7c8c(cccc68)-c6ccccc6S7)c5)c4cc32)cc1 ; c1cc(-c2ccc3c4c(cccc24)-c2ccccc2S3)cc(-n2c3ccccc3c3ccc4ccccc4c32)c1 ; c1ccc(-n2c3ccccc3c3cc4ccccc4c(-c4cccc(-c5ccc6c7c(cccc57)-c5ccccc5S6)c4)c32)cc1 ; c1cc(-c2ccc3c4c(cccc24)-c2ccccc2S3)cc(-n2c3ccccc3c3cc4ccccc4cc32)c1]

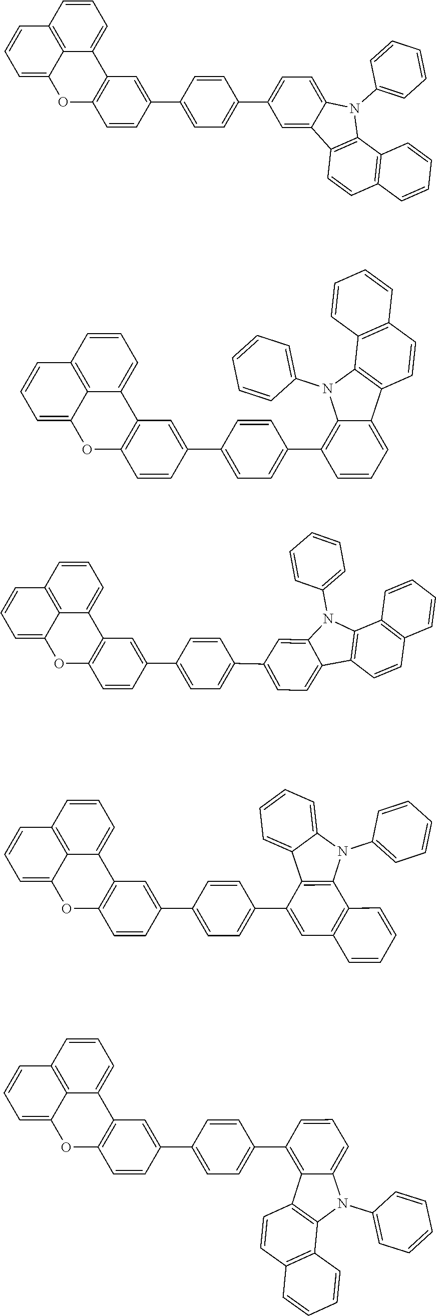 [SMILES: c1ccc(-n2c3ccc(-c4ccc(-c5ccc6c(c5)-c5cccc7cccc(c57)O6)cc4)cc3c3ccc4ccccc4c32)cc1 ; c1ccc(-n2c3c(-c4ccc(-c5ccc6c(c5)-c5cccc7cccc(c57)O6)cc4)cccc3c3ccc4ccccc4c32)cc1 ; c1ccc(-n2c3cc(-c4ccc(-c5ccc6c(c5)-c5cccc7cccc(c57)O6)cc4)ccc3c3ccc4ccccc4c32)cc1 ; c1ccc(-n2c3ccccc3c3c(-c4ccc(-c5ccc6c(c5)-c5cccc7cccc(c57)O6)cc4)cc4ccccc4c32)cc1 ; c1ccc(-n2c3cccc(-c4ccc(-c5ccc6c(c5)-c5cccc7cccc(c57)O6)cc4)c3c3ccc4ccccc4c32)cc1]

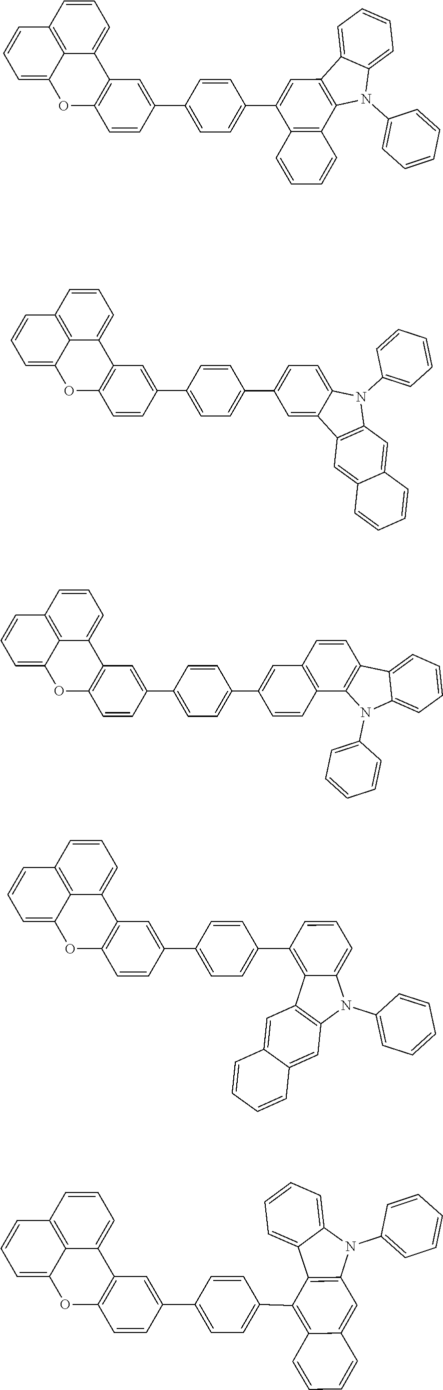 [SMILES: c1ccc(-n2c3ccccc3c3cc(-c4ccc(-c5ccc6c(c5)-c5cccc7cccc(c57)O6)cc4)c4ccccc4c32)cc1 ; c1ccc(-n2c3ccc(-c4ccc(-c5ccc6c(c5)-c5cccc7cccc(c57)O6)cc4)cc3c3cc4ccccc4cc32)cc1 ; c1ccc(-n2c3ccccc3c3ccc4cc(-c5ccc(-c6ccc7c(c6)-c6cccc8cccc(c68)O7)cc5)ccc4c32)cc1 ; c1ccc(-n2c3cc4ccccc4cc3c3c(-c4ccc(-c5ccc6c(c5)-c5cccc7cccc(c57)O6)cc4)cccc32)cc1 ; c1ccc(-n2c3ccccc3c3c(-c4ccc(-c5ccc6c(c5)-c5cccc7cccc(c57)O6)cc4)c4ccccc4cc32)cc1]

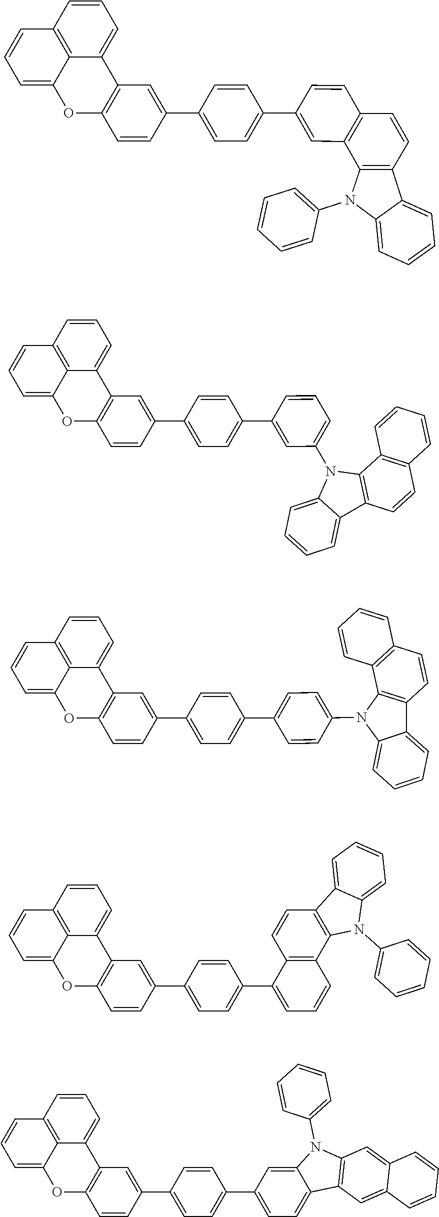 [SMILES: c1ccc(-n2c3ccccc3c3ccc4ccc(-c5ccc(-c6ccc7c(c6)-c6cccc8cccc(c68)O7)cc5)cc4c32)cc1 ; c1cc(-c2ccc(-c3ccc4c(c3)-c3cccc5cccc(c35)O4)cc2)cc(-n2c3ccccc3c3ccc4ccccc4c32)c1 ; c1cc2c3c(cccc3c1)-c1cc(-c3ccc(-c4ccc(-n5c6ccccc6c6ccc7ccccc7c65)cc4)cc3)ccc1O2 ; c1ccc(-n2c3ccccc3c3ccc4c(-c5ccc(-c6ccc7c(c6)-c6cccc8cccc(c68)O7)cc5)cccc4c32)cc1 ; c1ccc(-n2c3cc(-c4ccc(-c5ccc6c(c5)-c5cccc7cccc(c57)O6)cc4)ccc3c3cc4ccccc4cc32)cc1]

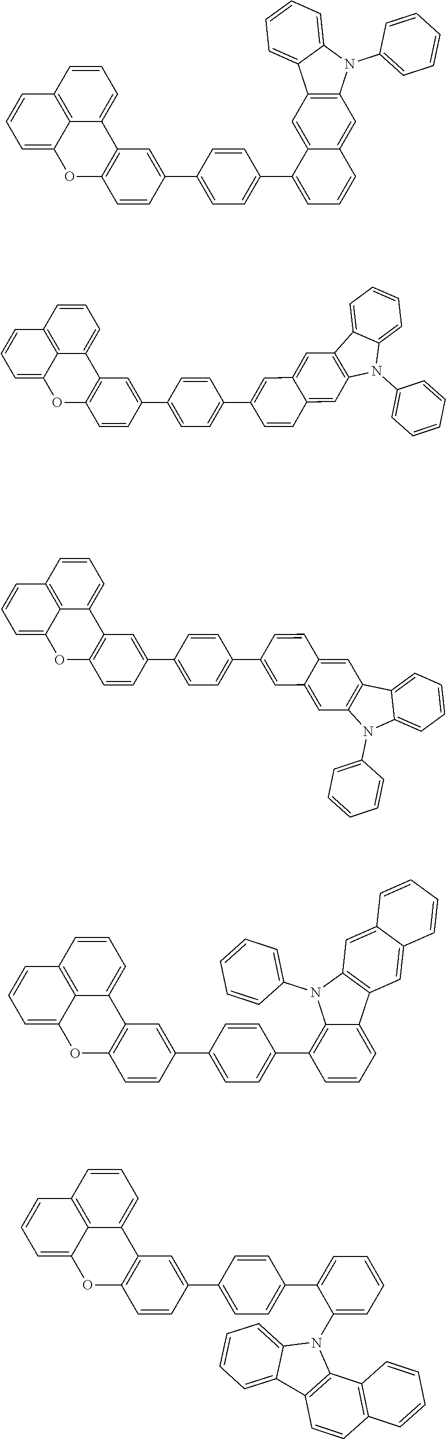 [SMILES: c1ccc(-n2c3ccccc3c3cc4c(-c5ccc(-c6ccc7c(c6)-c6cccc8cccc(c68)O7)cc5)cccc4cc32)cc1 ; c1ccc(-n2c3ccccc3c3cc4cc(-c5ccc(-c6ccc7c(c6)-c6cccc8cccc(c68)O7)cc5)ccc4cc32)cc1 ; c1ccc(-n2c3ccccc3c3cc4ccc(-c5ccc(-c6ccc7c(c6)-c6cccc8cccc(c68)O7)cc5)cc4cc32)cc1 ; c1ccc(-n2c3cc4ccccc4cc3c3cccc(-c4ccc(-c5ccc6c(c5)-c5cccc7cccc(c57)O6)cc4)c32)cc1 ; c1ccc(-n2c3ccccc3c3ccc4ccccc4c32)c(-c2ccc(-c3ccc4c(c3)-c3cccc5cccc(c35)O4)cc2)c1]

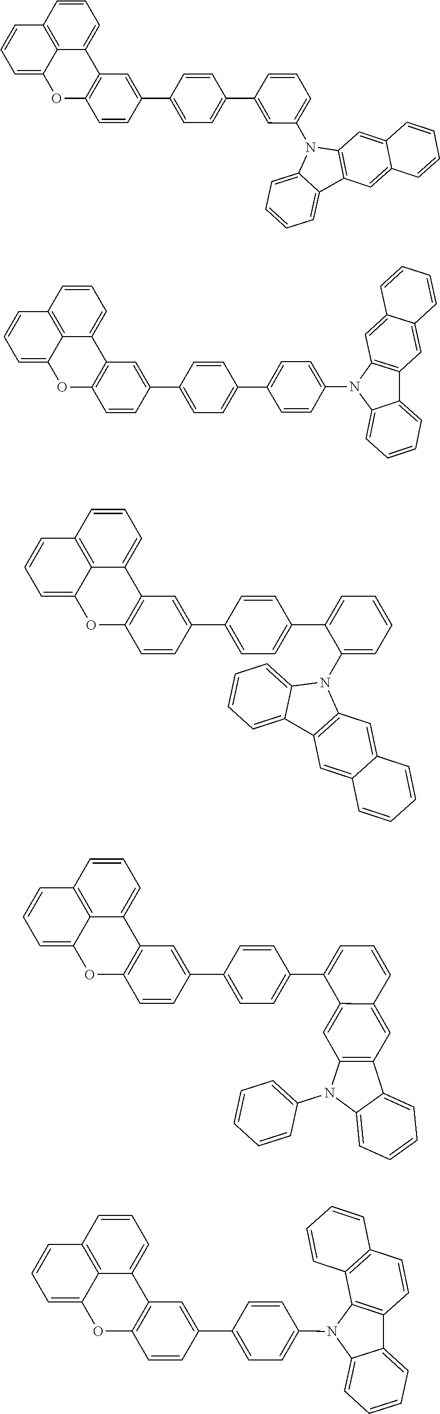 [SMILES: c1cc(-c2ccc(-c3ccc4c(c3)-c3cccc5cccc(c35)O4)cc2)cc(-n2c3ccccc3c3cc4ccccc4cc32)c1 ; c1ccc2cc3c(cc2c1)c1ccccc1n3-c1ccc(-c2ccc(-c3ccc4c(c3)-c3cccc5cccc(c35)O4)cc2)cc1 ; c1ccc(-n2c3ccccc3c3cc4ccccc4cc32)c(-c2ccc(-c3ccc4c(c3)-c3cccc5cccc(c35)O4)cc2)c1 ; c1ccc(-n2c3ccccc3c3cc4cccc(-c5ccc(-c6ccc7c(c6)-c6cccc8cccc(c68)O7)cc5)c4cc32)cc1 ; c1cc2c3c(cccc3c1)-c1cc(-c3ccc(-n4c5ccccc5c5ccc6ccccc6c54)cc3)ccc1O2]

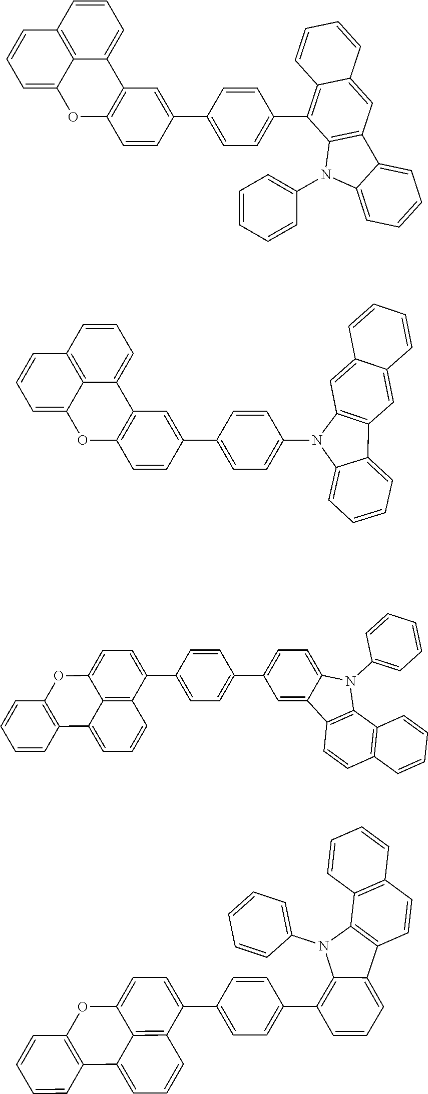 [SMILES: c1ccc(-n2c3ccccc3c3cc4ccccc4c(-c4ccc(-c5ccc6c(c5)-c5cccc7cccc(c57)O6)cc4)c32)cc1 ; c1ccc2cc3c(cc2c1)c1ccccc1n3-c1ccc(-c2ccc3c(c2)-c2cccc4cccc(c24)O3)cc1 ; c1ccc(-n2c3ccc(-c4ccc(-c5ccc6c7c(cccc57)-c5ccccc5O6)cc4)cc3c3ccc4ccccc4c32)cc1 ; c1ccc(-n2c3c(-c4ccc(-c5ccc6c7c(cccc57)-c5ccccc5O6)cc4)cccc3c3ccc4ccccc4c32)cc1]

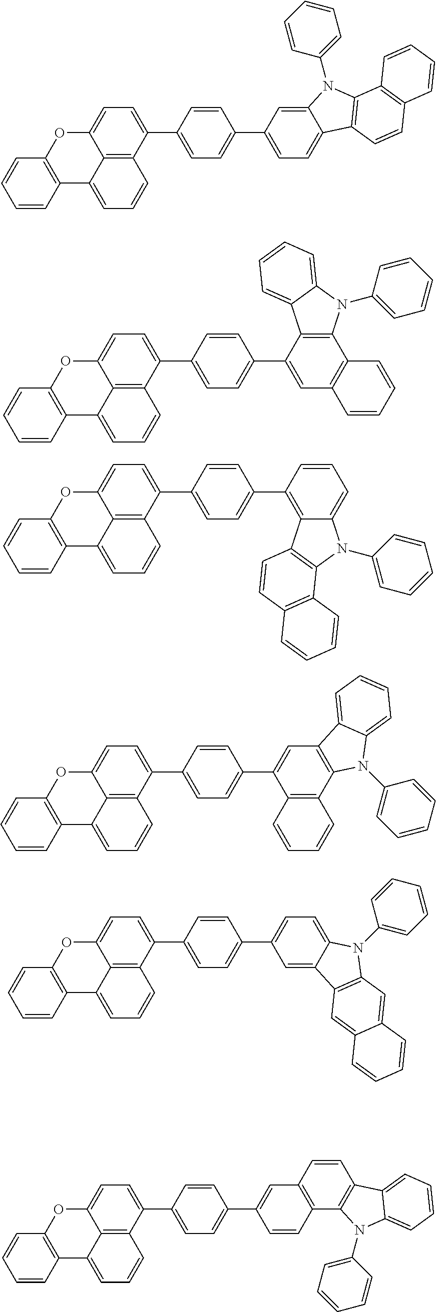

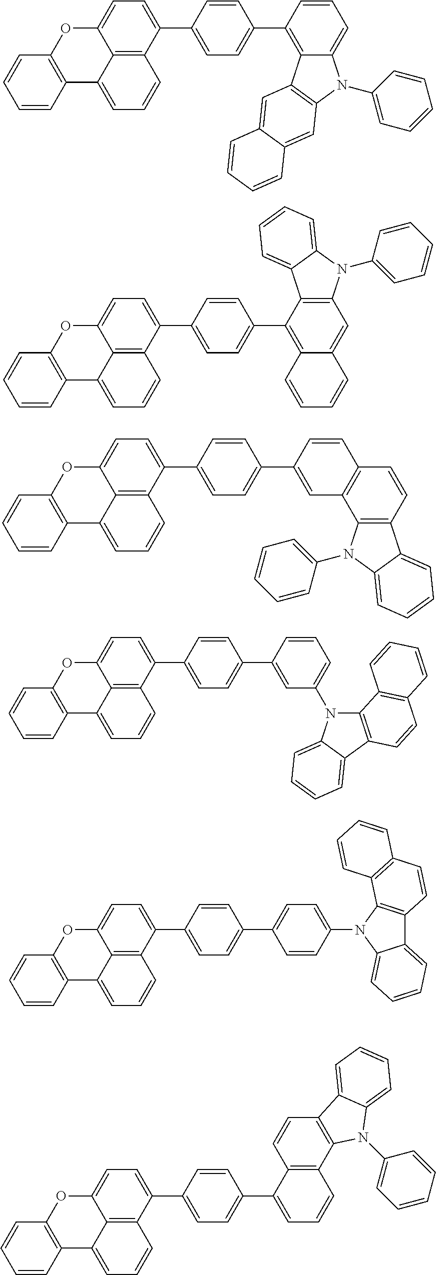

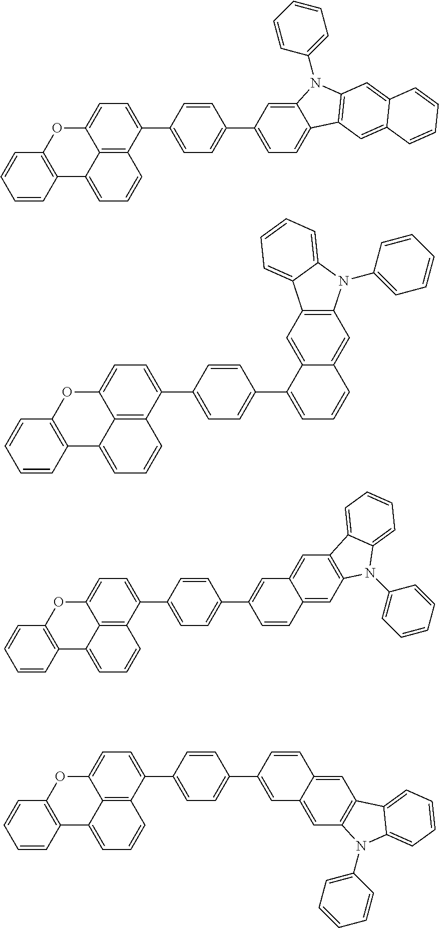 [SMILES: c1ccc(-n2c3cc(-c4ccc(-c5ccc6c7c(cccc57)-c5ccccc5O6)cc4)ccc3c3cc4ccccc4cc32)cc1 ; c1ccc(-n2c3ccccc3c3cc4c(-c5ccc(-c6ccc7c8c(cccc68)-c6ccccc6O7)cc5)cccc4cc32)cc1 ; c1ccc(-n2c3ccccc3c3cc4cc(-c5ccc(-c6ccc7c8c(cccc68)-c6ccccc6O7)cc5)ccc4cc32)cc1 ; c1ccc(-n2c3ccccc3c3cc4ccc(-c5ccc(-c6ccc7c8c(cccc68)-c6ccccc6O7)cc5)cc4cc32)cc1]

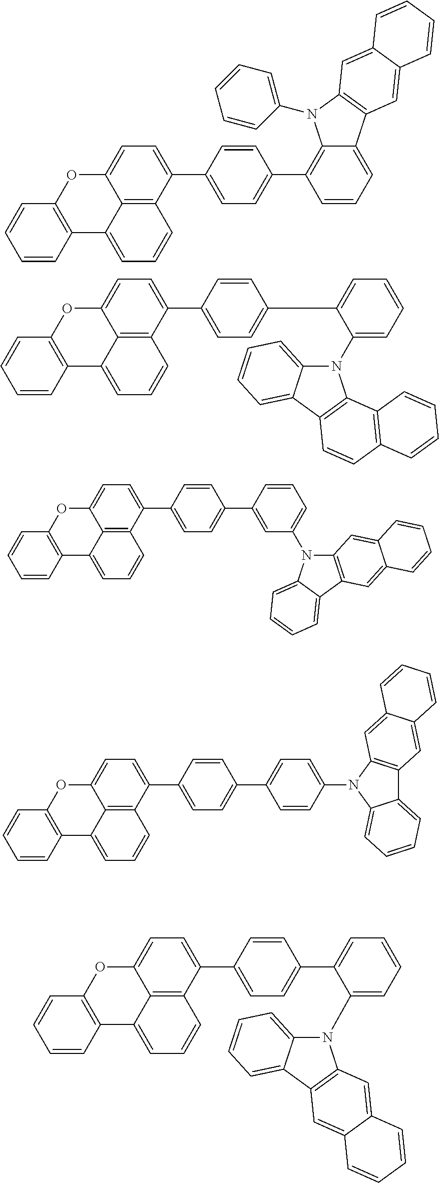

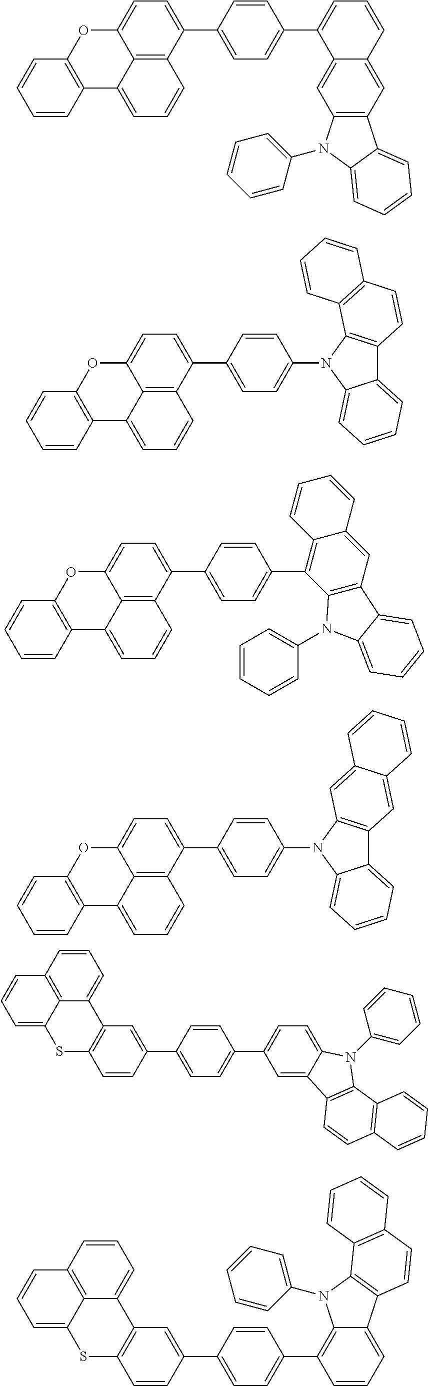

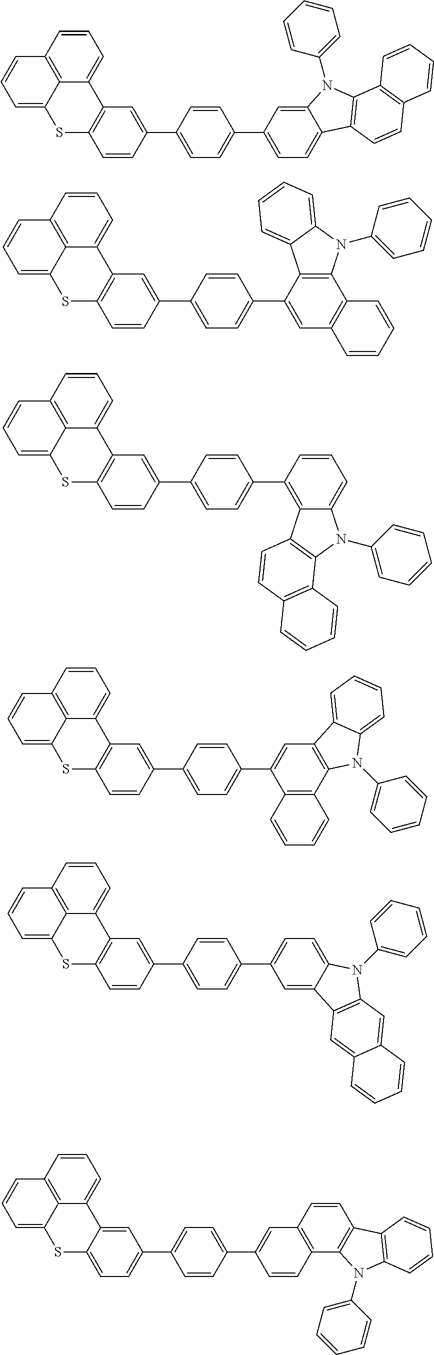

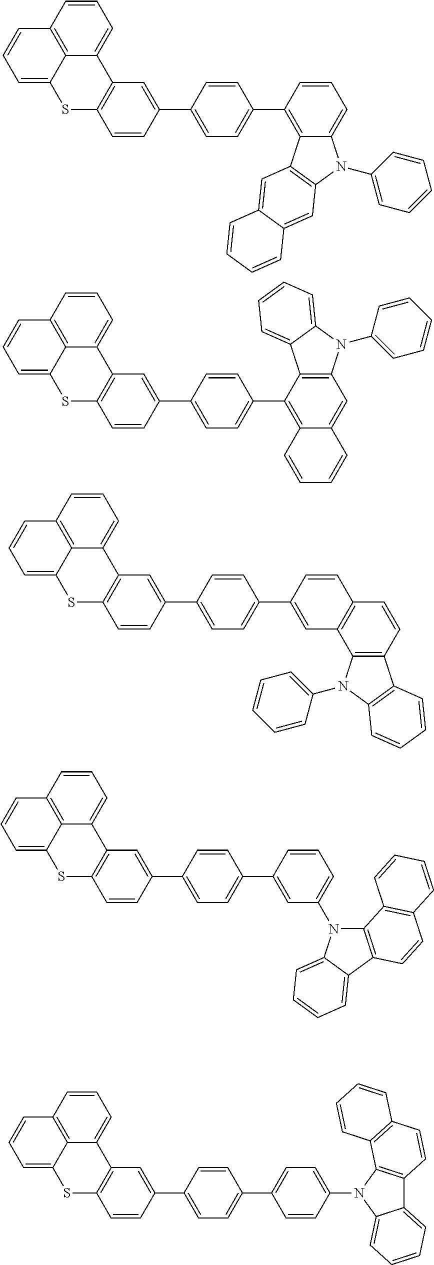

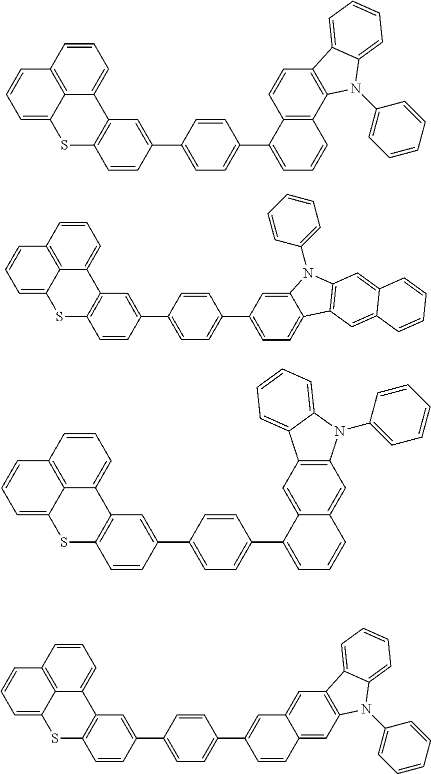

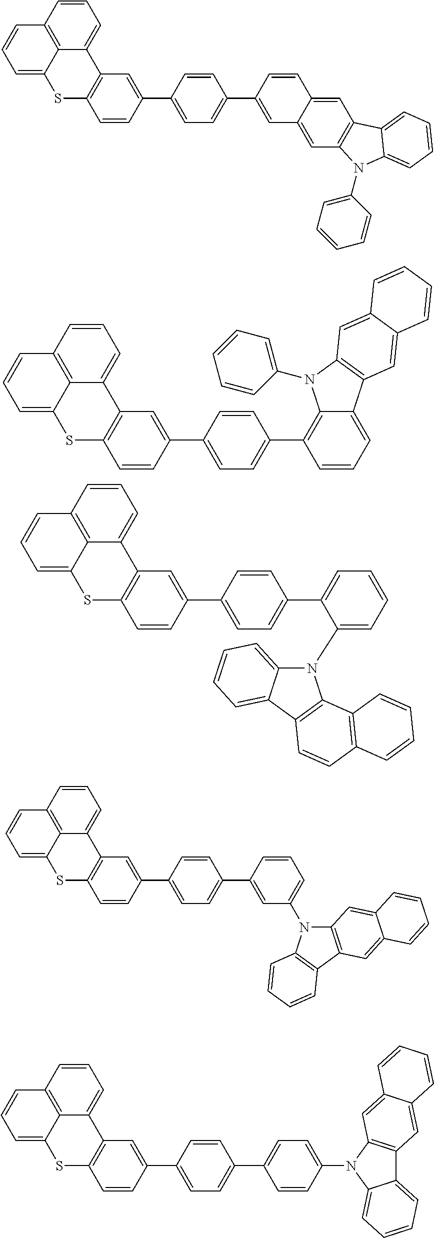

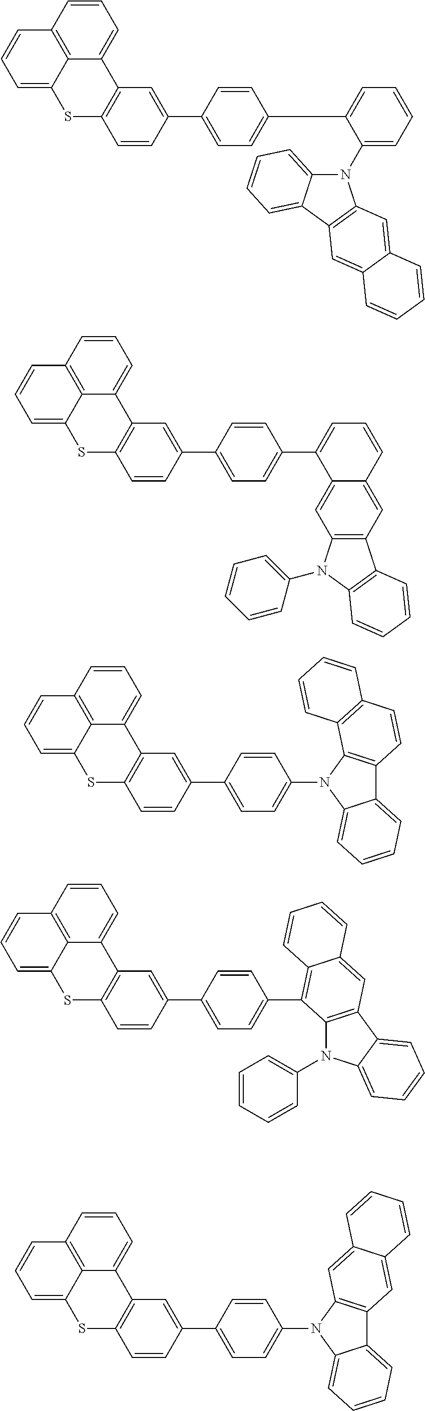

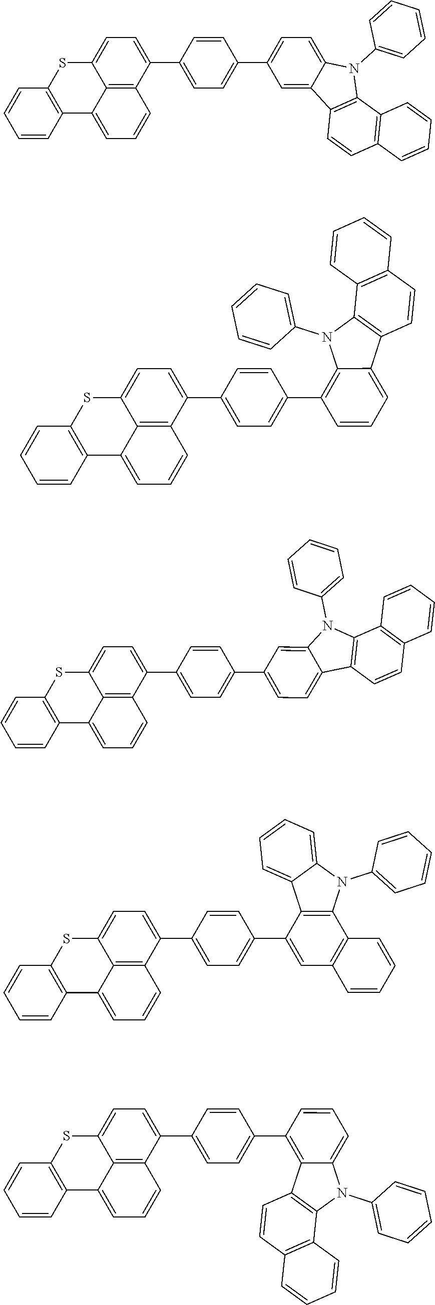 [SMILES: c1ccc(-n2c3ccc(-c4ccc(-c5ccc6c7c(cccc57)-c5ccccc5S6)cc4)cc3c3ccc4ccccc4c32)cc1 ; c1ccc(-n2c3c(-c4ccc(-c5ccc6c7c(cccc57)-c5ccccc5S6)cc4)cccc3c3ccc4ccccc4c32)cc1 ; c1ccc(-n2c3cc(-c4ccc(-c5ccc6c7c(cccc57)-c5ccccc5S6)cc4)ccc3c3ccc4ccccc4c32)cc1 ; c1ccc(-n2c3ccccc3c3c(-c4ccc(-c5ccc6c7c(cccc57)-c5ccccc5S6)cc4)cc4ccccc4c32)cc1 ; c1ccc(-n2c3cccc(-c4ccc(-c5ccc6c7c(cccc57)-c5ccccc5S6)cc4)c3c3ccc4ccccc4c32)cc1]

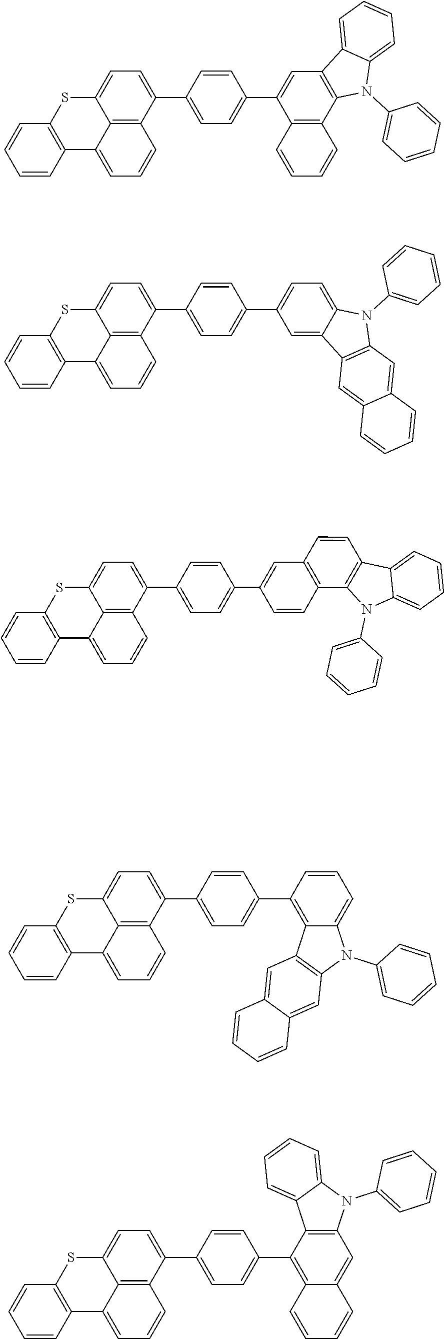 [SMILES: c1ccc(-n2c3ccccc3c3cc(-c4ccc(-c5ccc6c7c(cccc57)-c5ccccc5S6)cc4)c4ccccc4c32)cc1 ; c1ccc(-n2c3ccc(-c4ccc(-c5ccc6c7c(cccc57)-c5ccccc5S6)cc4)cc3c3cc4ccccc4cc32)cc1 ; c1ccc(-n2c3ccccc3c3ccc4cc(-c5ccc(-c6ccc7c8c(cccc68)-c6ccccc6S7)cc5)ccc4c32)cc1 ; c1ccc(-n2c3cc4ccccc4cc3c3c(-c4ccc(-c5ccc6c7c(cccc57)-c5ccccc5S6)cc4)cccc32)cc1 ; c1ccc(-n2c3ccccc3c3c(-c4ccc(-c5ccc6c7c(cccc57)-c5ccccc5S6)cc4)c4ccccc4cc32)cc1]

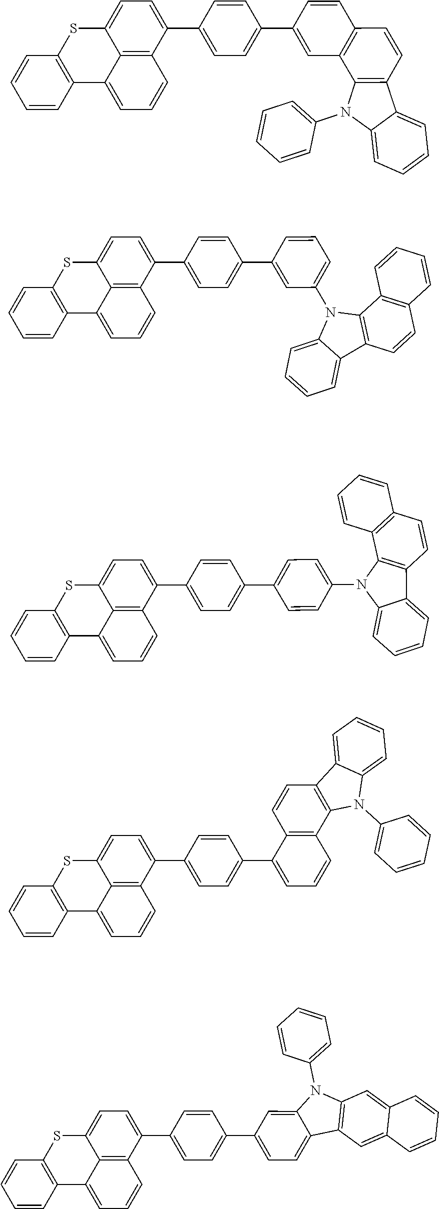 [SMILES: c1ccc(-n2c3ccccc3c3ccc4ccc(-c5ccc(-c6ccc7c8c(cccc68)-c6ccccc6S7)cc5)cc4c32)cc1 ; c1cc(-c2ccc(-c3ccc4c5c(cccc35)-c3ccccc3S4)cc2)cc(-n2c3ccccc3c3ccc4ccccc4c32)c1 ; c1ccc2c(c1)Sc1ccc(-c3ccc(-c4ccc(-n5c6ccccc6c6ccc7ccccc7c65)cc4)cc3)c3cccc-2c13 ; c1ccc(-n2c3ccccc3c3ccc4c(-c5ccc(-c6ccc7c8c(cccc68)-c6ccccc6S7)cc5)cccc4c32)cc1 ; c1ccc(-n2c3cc(-c4ccc(-c5ccc6c7c(cccc57)-c5ccccc5S6)cc4)ccc3c3cc4ccccc4cc32)cc1]

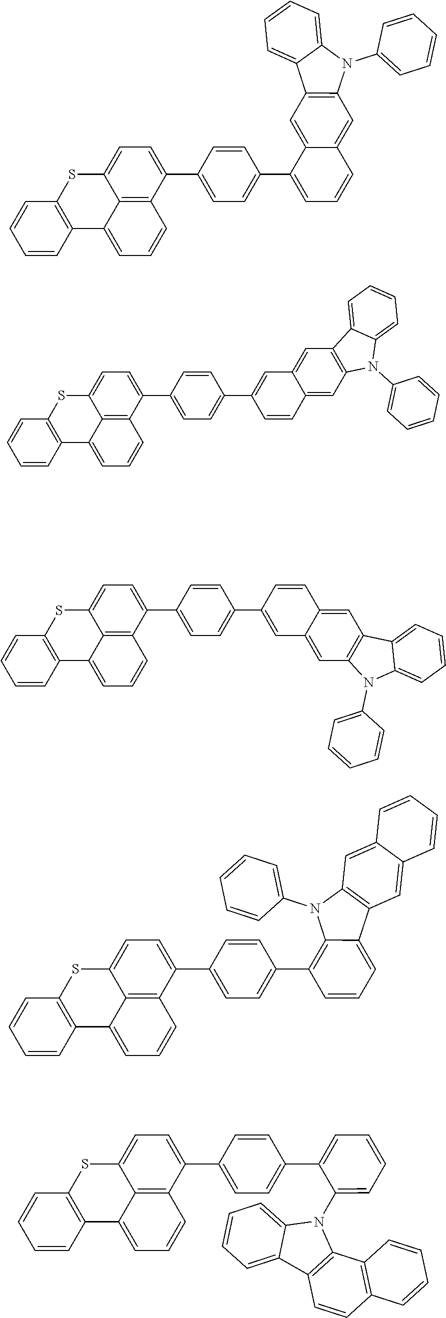 [SMILES: c1ccc(-n2c3ccccc3c3cc4c(-c5ccc(-c6ccc7c8c(cccc68)-c6ccccc6S7)cc5)cccc4cc32)cc1 ; c1ccc(-n2c3ccccc3c3cc4cc(-c5ccc(-c6ccc7c8c(cccc68)-c6ccccc6S7)cc5)ccc4cc32)cc1 ; c1ccc(-n2c3ccccc3c3cc4ccc(-c5ccc(-c6ccc7c8c(cccc68)-c6ccccc6S7)cc5)cc4cc32)cc1 ; c1ccc(-n2c3cc4ccccc4cc3c3cccc(-c4ccc(-c5ccc6c7c(cccc57)-c5ccccc5S6)cc4)c32)cc1 ; c1ccc2c(c1)Sc1ccc(-c3ccc(-c4ccccc4-n4c5ccccc5c5ccc6ccccc6c54)cc3)c3cccc-2c13]

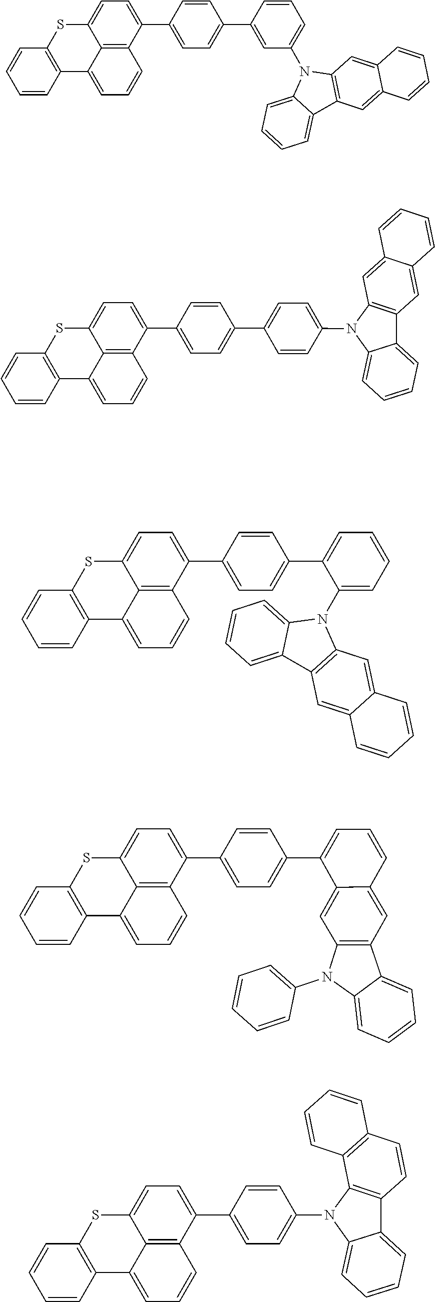 [SMILES: c1cc(-c2ccc(-c3ccc4c5c(cccc35)-c3ccccc3S4)cc2)cc(-n2c3ccccc3c3cc4ccccc4cc32)c1 ; c1ccc2c(c1)Sc1ccc(-c3ccc(-c4ccc(-n5c6ccccc6c6cc7ccccc7cc65)cc4)cc3)c3cccc-2c13 ; c1ccc2c(c1)Sc1ccc(-c3ccc(-c4ccccc4-n4c5ccccc5c5cc6ccccc6cc54)cc3)c3cccc-2c13 ; c1ccc(-n2c3ccccc3c3cc4cccc(-c5ccc(-c6ccc7c8c(cccc68)-c6ccccc6S7)cc5)c4cc32)cc1 ; c1ccc2c(c1)Sc1ccc(-c3ccc(-n4c5ccccc5c5ccc6ccccc6c54)cc3)c3cccc-2c13]

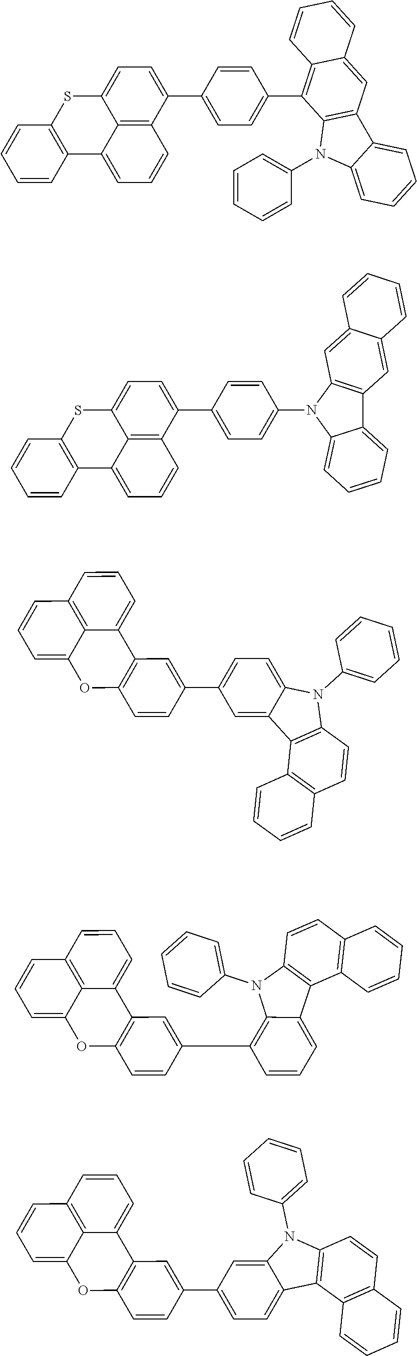 [SMILES: c1ccc(-n2c3ccccc3c3cc4ccccc4c(-c4ccc(-c5ccc6c7c(cccc57)-c5ccccc5S6)cc4)c32)cc1 ; c1ccc2c(c1)Sc1ccc(-c3ccc(-n4c5ccccc5c5cc6ccccc6cc54)cc3)c3cccc-2c13 ; c1ccc(-n2c3ccc(-c4ccc5c(c4)-c4cccc6cccc(c46)O5)cc3c3c4ccccc4ccc32)cc1 ; c1ccc(-n2c3ccc4ccccc4c3c3cccc(-c4ccc5c(c4)-c4cccc6cccc(c46)O5)c32)cc1 ; c1ccc(-n2c3cc(-c4ccc5c(c4)-c4cccc6cccc(c46)O5)ccc3c3c4ccccc4ccc32)cc1]

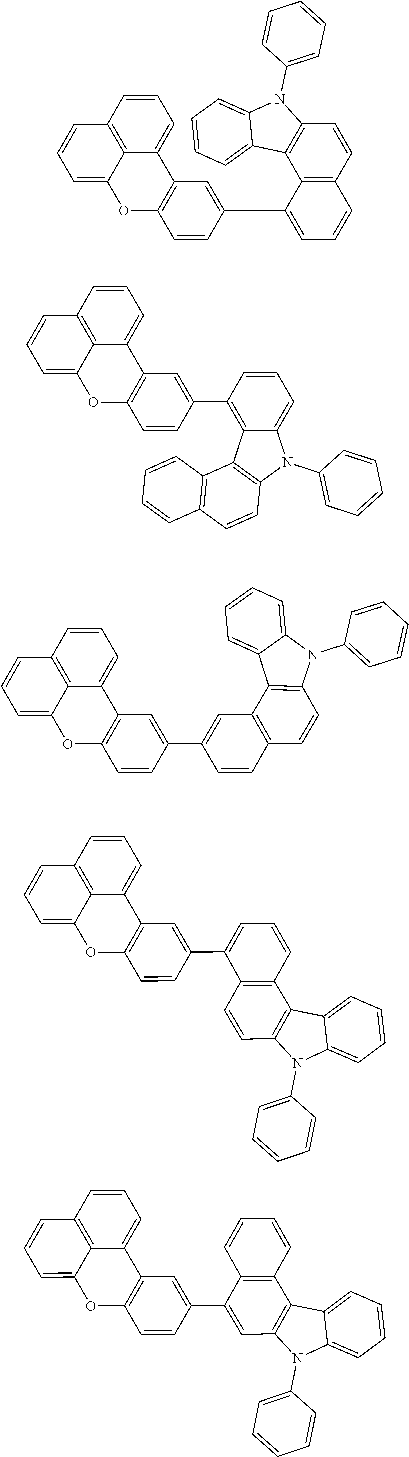 [SMILES: c1ccc(-n2c3ccccc3c3c4c(-c5ccc6c(c5)-c5cccc7cccc(c57)O6)cccc4ccc32)cc1 ; c1ccc(-n2c3cccc(-c4ccc5c(c4)-c4cccc6cccc(c46)O5)c3c3c4ccccc4ccc32)cc1 ; c1ccc(-n2c3ccccc3c3c4cc(-c5ccc6c(c5)-c5cccc7cccc(c57)O6)ccc4ccc32)cc1 ; c1ccc(-n2c3ccccc3c3c4cccc(-c5ccc6c(c5)-c5cccc7cccc(c57)O6)c4ccc32)cc1 ; c1ccc(-n2c3ccccc3c3c4ccccc4c(-c4ccc5c(c4)-c4cccc6cccc(c46)O5)cc32)cc1]

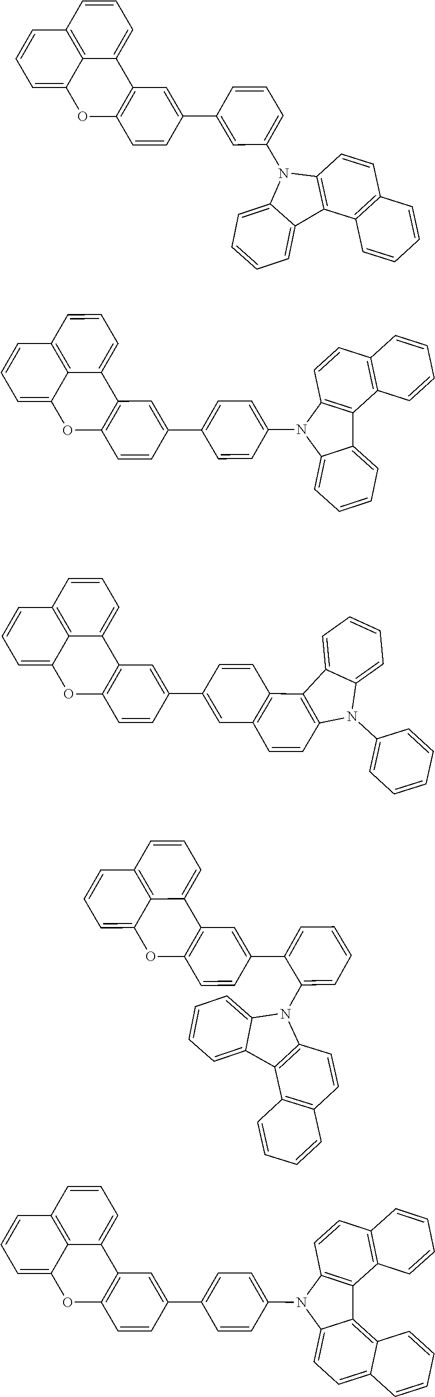 [SMILES: c1cc(-c2ccc3c(c2)-c2cccc4cccc(c24)O3)cc(-n2c3ccccc3c3c4ccccc4ccc32)c1 ; c1cc2c3c(cccc3c1)-c1cc(-c3ccc(-n4c5ccccc5c5c6ccccc6ccc54)cc3)ccc1O2 ; c1ccc(-n2c3ccccc3c3c4ccc(-c5ccc6c(c5)-c5cccc7cccc(c57)O6)cc4ccc32)cc1 ; c1ccc(-n2c3ccccc3c3c4ccccc4ccc32)c(-c2ccc3c(c2)-c2cccc4cccc(c24)O3)c1 ; c1cc2c3c(cccc3c1)-c1cc(-c3ccc(-n4c5ccc6ccccc6c5c5c6ccccc6ccc54)cc3)ccc1O2]

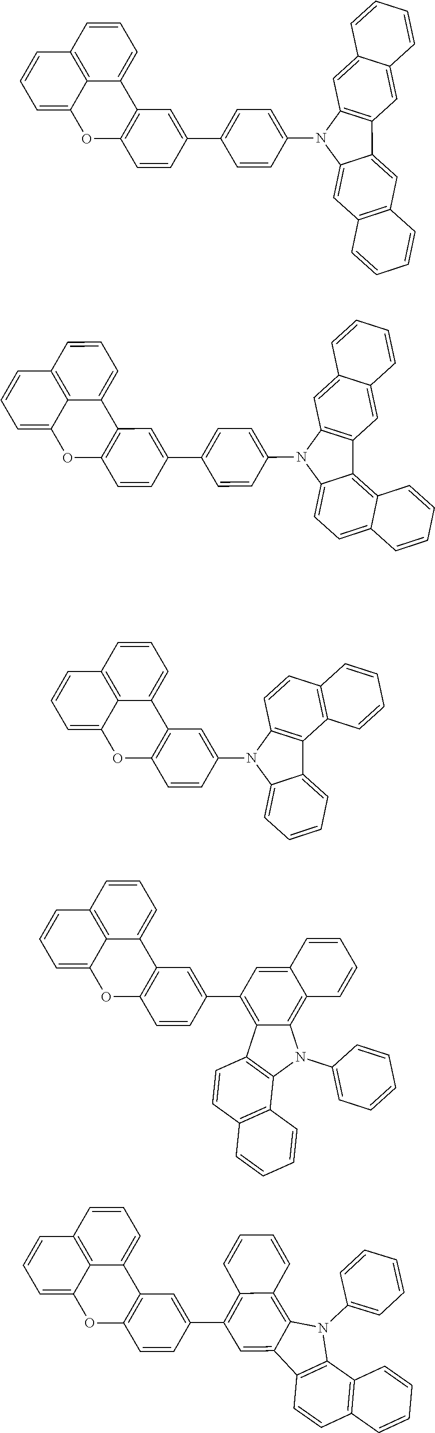 [SMILES: c1ccc2cc3c(cc2c1)c1cc2ccccc2cc1n3-c1ccc(-c2ccc3c(c2)-c2cccc4cccc(c24)O3)cc1 ; c1ccc2cc3c(cc2c1)c1c2ccccc2ccc1n3-c1ccc(-c2ccc3c(c2)-c2cccc4cccc(c24)O3)cc1 ; c1cc2c3c(cccc3c1)-c1cc(-n3c4ccccc4c4c5ccccc5ccc43)ccc1O2 ; c1ccc(-n2c3c4ccccc4ccc3c3c(-c4ccc5c(c4)-c4cccc6cccc(c46)O5)cc4ccccc4c32)cc1 ; c1ccc(-n2c3c4ccccc4ccc3c3cc(-c4ccc5c(c4)-c4cccc6cccc(c46)O5)c4ccccc4c32)cc1]

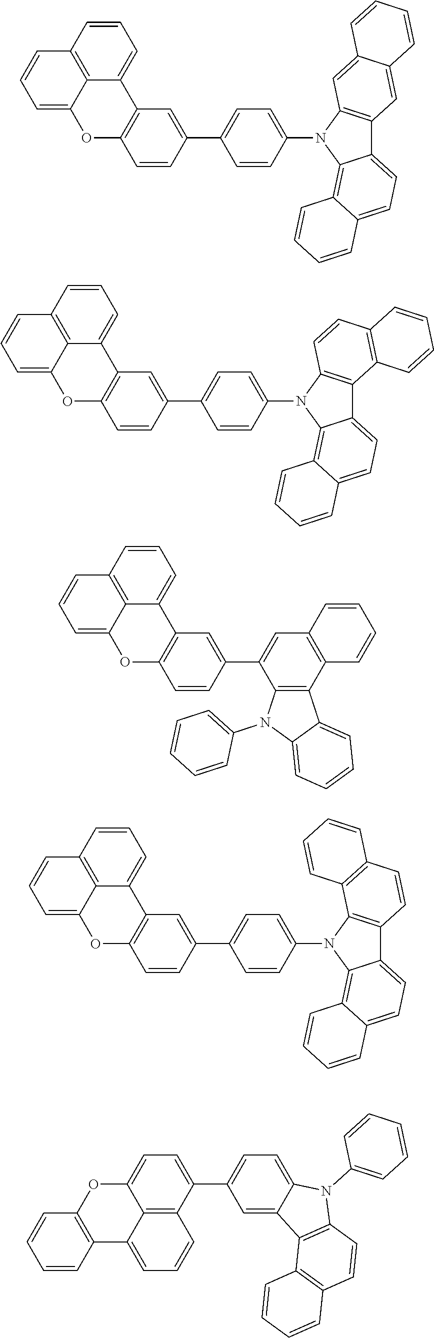

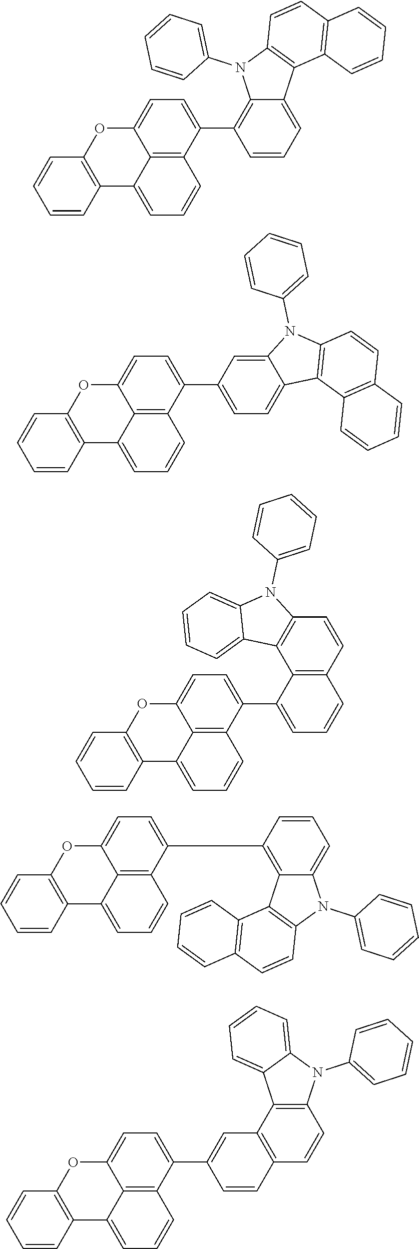

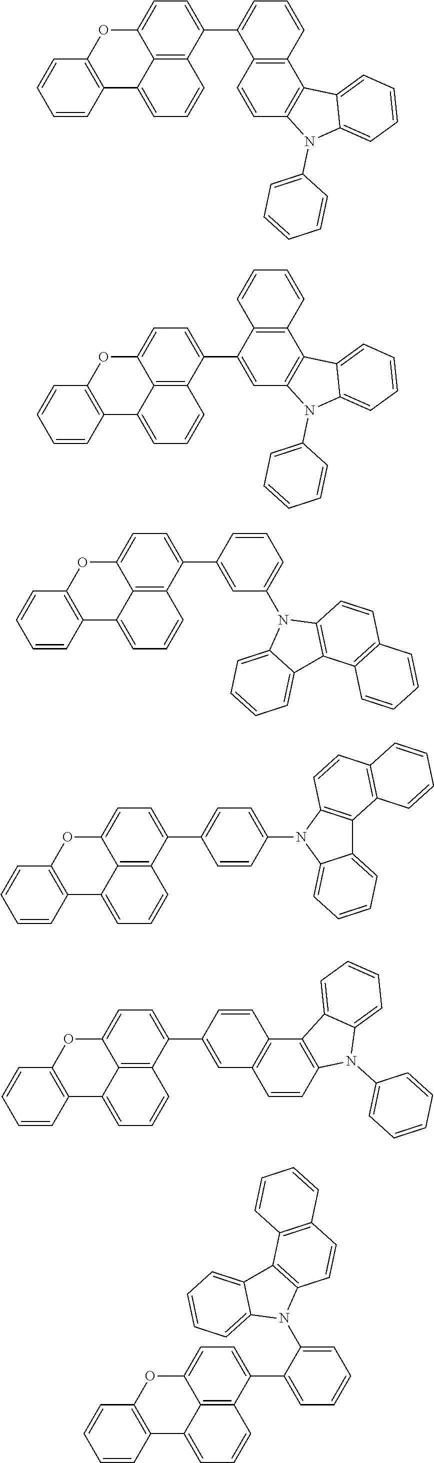

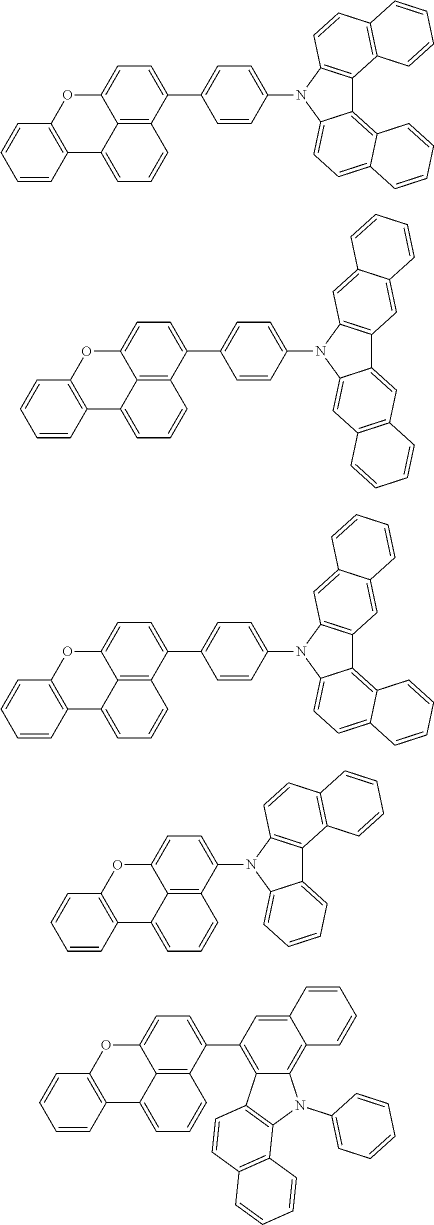

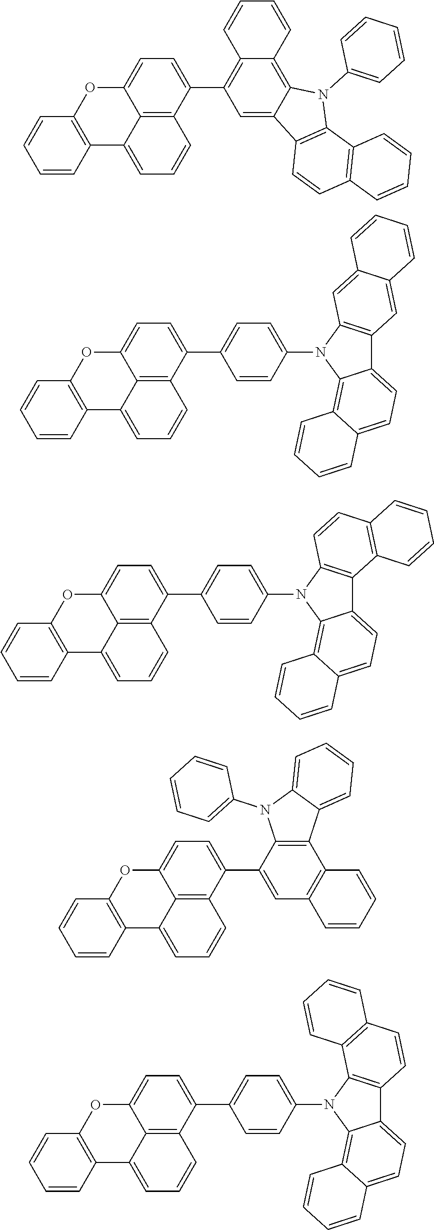

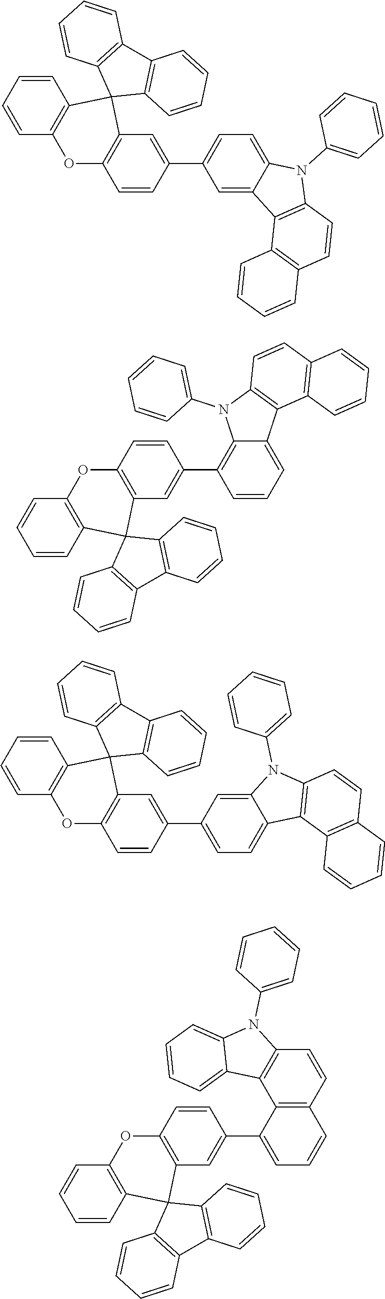

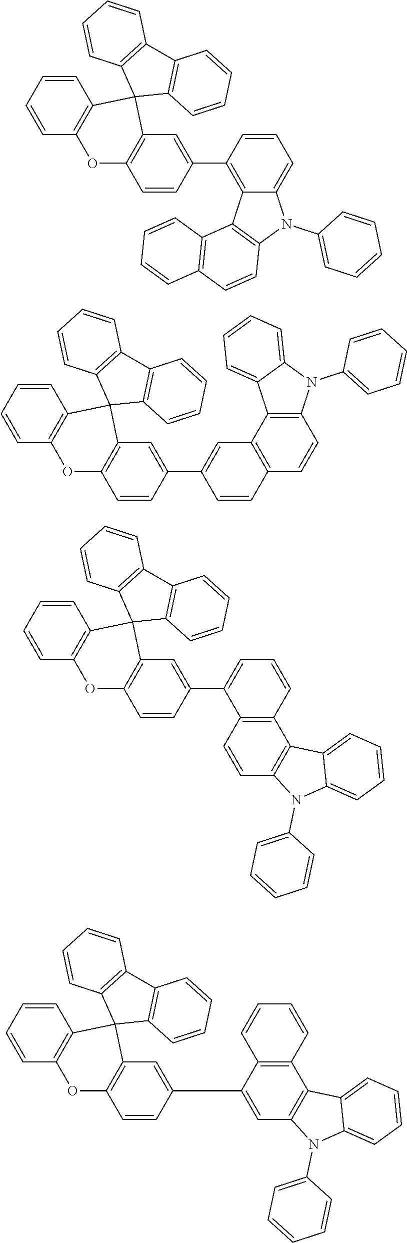

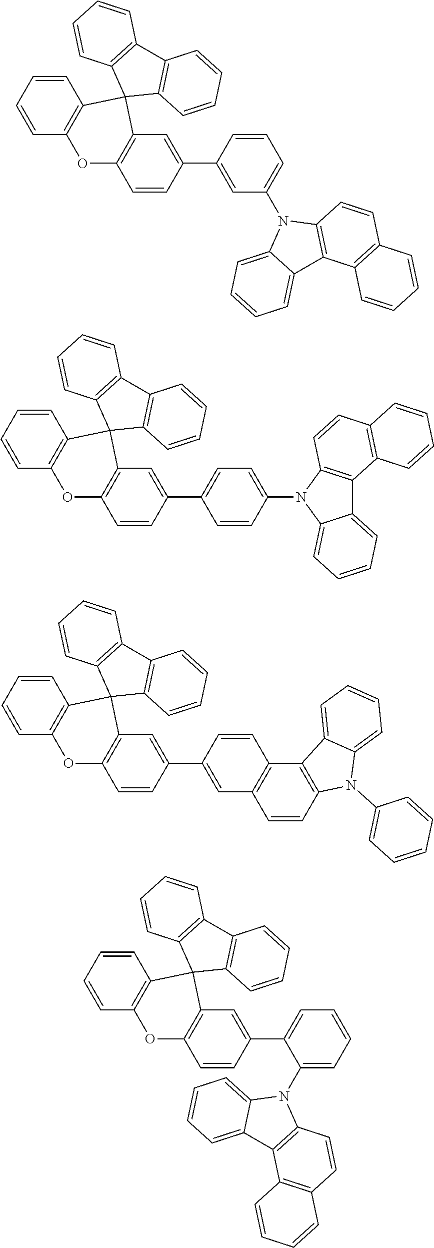 [SMILES: c1cc(-c2ccc3c(c2)C2(c4ccccc4O3)c3ccccc3-c3ccccc32)cc(-n2c3ccccc3c3c4ccccc4ccc32)c1 ; c1ccc2c(c1)Oc1ccc(-c3ccc(-n4c5ccccc5c5c6ccccc6ccc54)cc3)cc1C21c2ccccc2-c2ccccc21 ; c1ccc(-n2c3ccccc3c3c4ccc(-c5ccc6c(c5)C5(c7ccccc7O6)c6ccccc6-c6ccccc65)cc4ccc32)cc1 ; c1ccc2c(c1)Oc1ccc(-c3ccccc3-n3c4ccccc4c4c5ccccc5ccc43)cc1C21c2ccccc2-c2ccccc21]

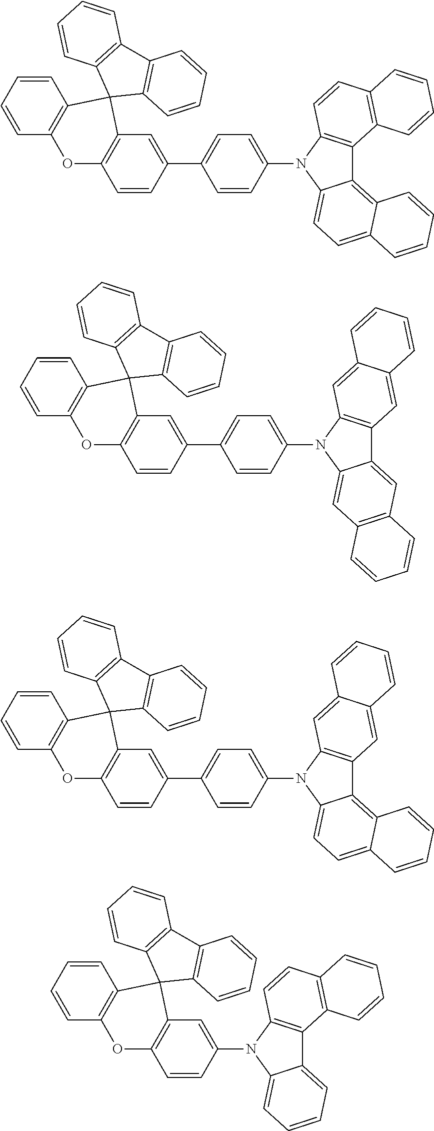 [SMILES: c1ccc2c(c1)Oc1ccc(-c3ccc(-n4c5ccc6ccccc6c5c5c6ccccc6ccc54)cc3)cc1C21c2ccccc2-c2ccccc21 ; c1ccc2c(c1)Oc1ccc(-c3ccc(-n4c5cc6ccccc6cc5c5cc6ccccc6cc54)cc3)cc1C21c2ccccc2-c2ccccc21 ; c1ccc2c(c1)Oc1ccc(-c3ccc(-n4c5cc6ccccc6cc5c5c6ccccc6ccc54)cc3)cc1C21c2ccccc2-c2ccccc21 ; c1ccc2c(c1)Oc1ccc(-n3c4ccccc4c4c5ccccc5ccc43)cc1C21c2ccccc2-c2ccccc21]

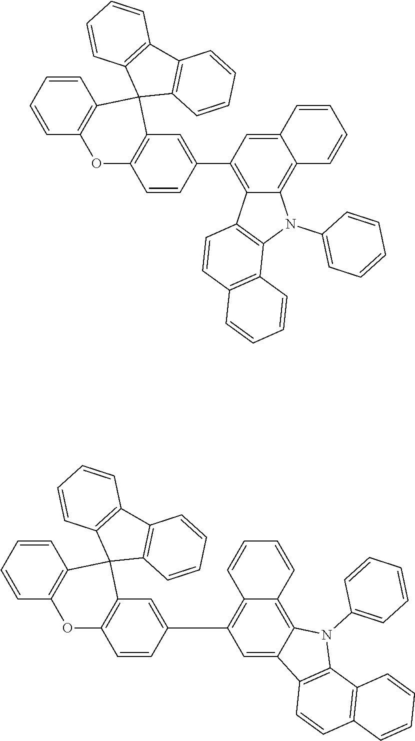 [SMILES: c1ccc(-n2c3c4ccccc4ccc3c3c(-c4ccc5c(c4)C4(c6ccccc6O5)c5ccccc5-c5ccccc54)cc4ccccc4c32)cc1 ; c1ccc(-n2c3c4ccccc4ccc3c3cc(-c4ccc5c(c4)C4(c6ccccc6O5)c5ccccc5-c5ccccc54)c4ccccc4c32)cc1]

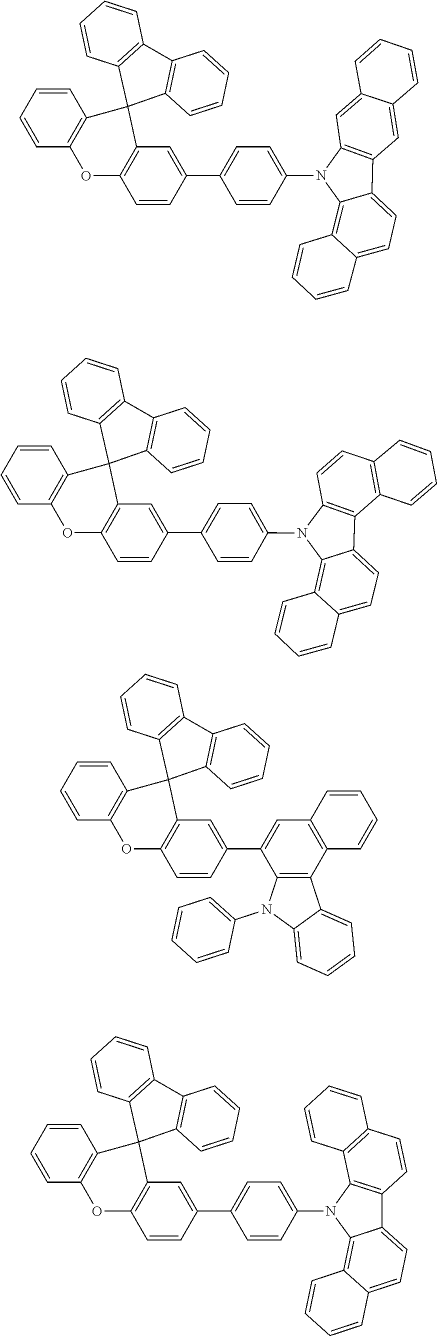 [SMILES: c1ccc2c(c1)Oc1ccc(-c3ccc(-n4c5cc6ccccc6cc5c5ccc6ccccc6c54)cc3)cc1C21c2ccccc2-c2ccccc21 ; c1ccc2c(c1)Oc1ccc(-c3ccc(-n4c5ccc6ccccc6c5c5ccc6ccccc6c54)cc3)cc1C21c2ccccc2-c2ccccc21 ; c1ccc(-n2c3ccccc3c3c4ccccc4cc(-c4ccc5c(c4)C4(c6ccccc6O5)c5ccccc5-c5ccccc54)c32)cc1 ; c1ccc2c(c1)Oc1ccc(-c3ccc(-n4c5c6ccccc6ccc5c5ccc6ccccc6c54)cc3)cc1C21c2ccccc2-c2ccccc21]

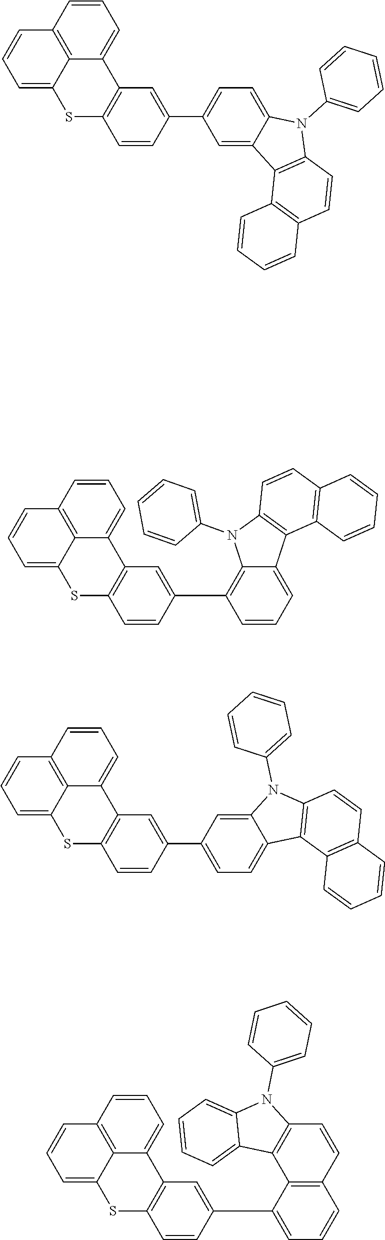 [SMILES: c1ccc(-n2c3ccc(-c4ccc5c(c4)-c4cccc6cccc(c46)S5)cc3c3c4ccccc4ccc32)cc1 ; c1ccc(-n2c3ccc4ccccc4c3c3cccc(-c4ccc5c(c4)-c4cccc6cccc(c46)S5)c32)cc1 ; c1ccc(-n2c3cc(-c4ccc5c(c4)-c4cccc6cccc(c46)S5)ccc3c3c4ccccc4ccc32)cc1 ; c1ccc(-n2c3ccccc3c3c4c(-c5ccc6c(c5)-c5cccc7cccc(c57)S6)cccc4ccc32)cc1]

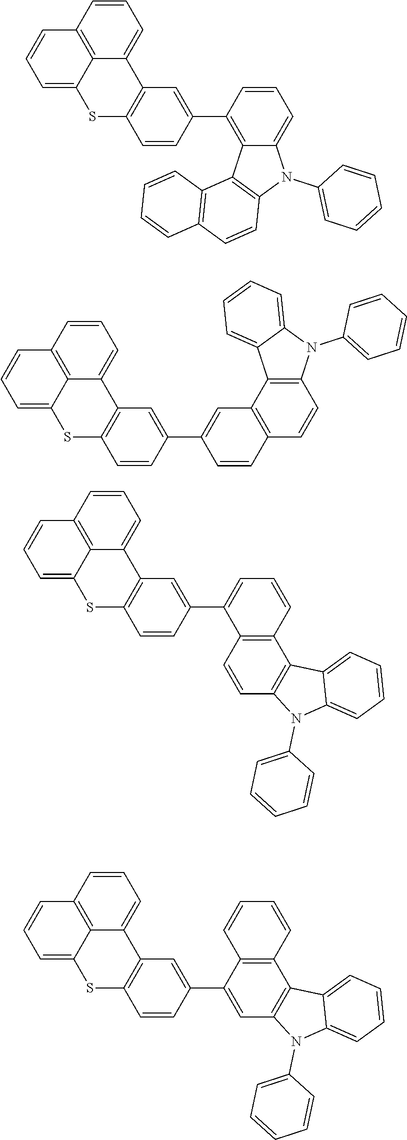

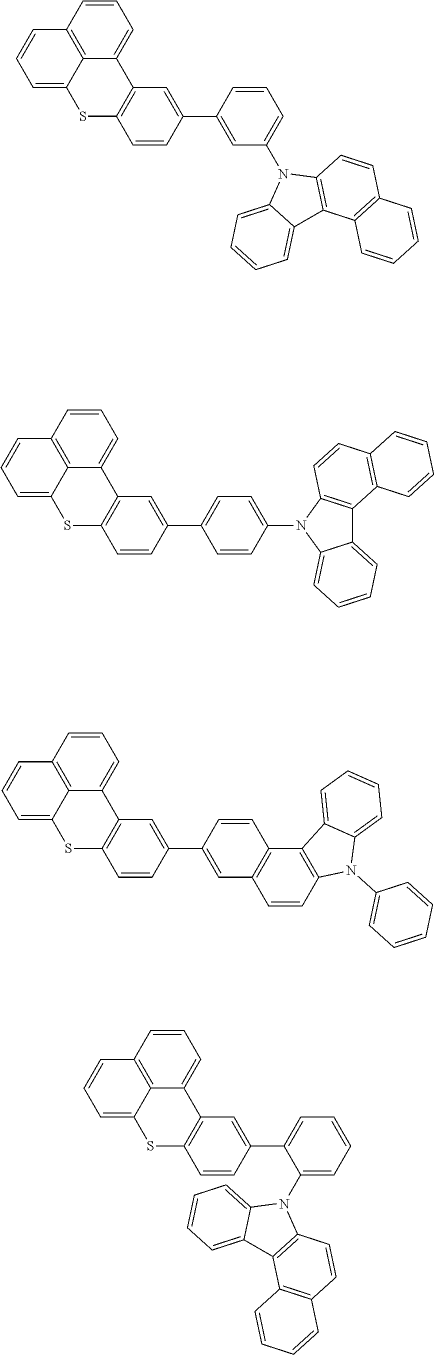 [SMILES: c1cc(-c2ccc3c(c2)-c2cccc4cccc(c24)S3)cc(-n2c3ccccc3c3c4ccccc4ccc32)c1 ; c1cc2c3c(cccc3c1)-c1cc(-c3ccc(-n4c5ccccc5c5c6ccccc6ccc54)cc3)ccc1S2 ; c1ccc(-n2c3ccccc3c3c4ccc(-c5ccc6c(c5)-c5cccc7cccc(c57)S6)cc4ccc32)cc1 ; c1ccc(-n2c3ccccc3c3c4ccccc4ccc32)c(-c2ccc3c(c2)-c2cccc4cccc(c24)S3)c1]

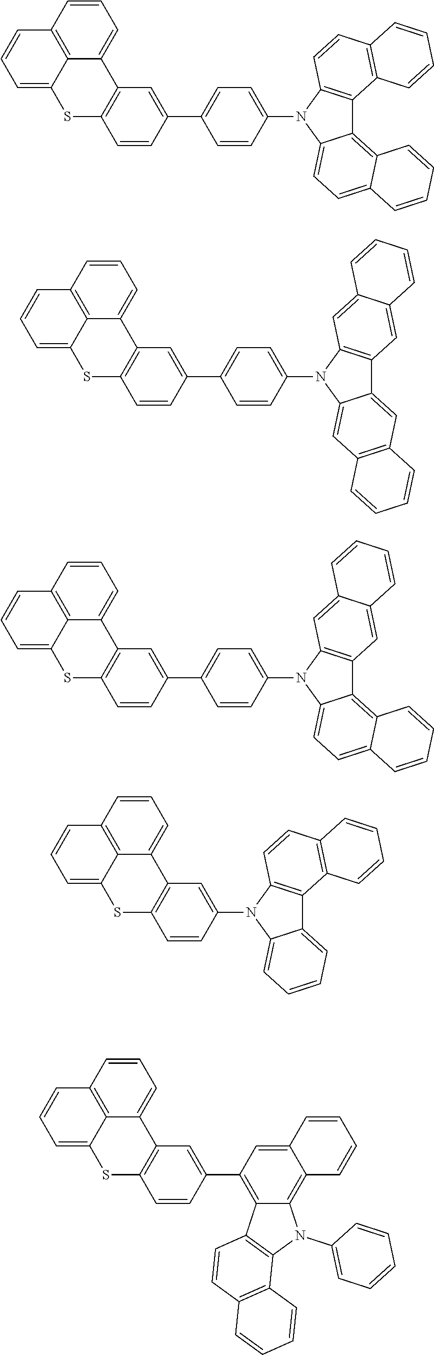

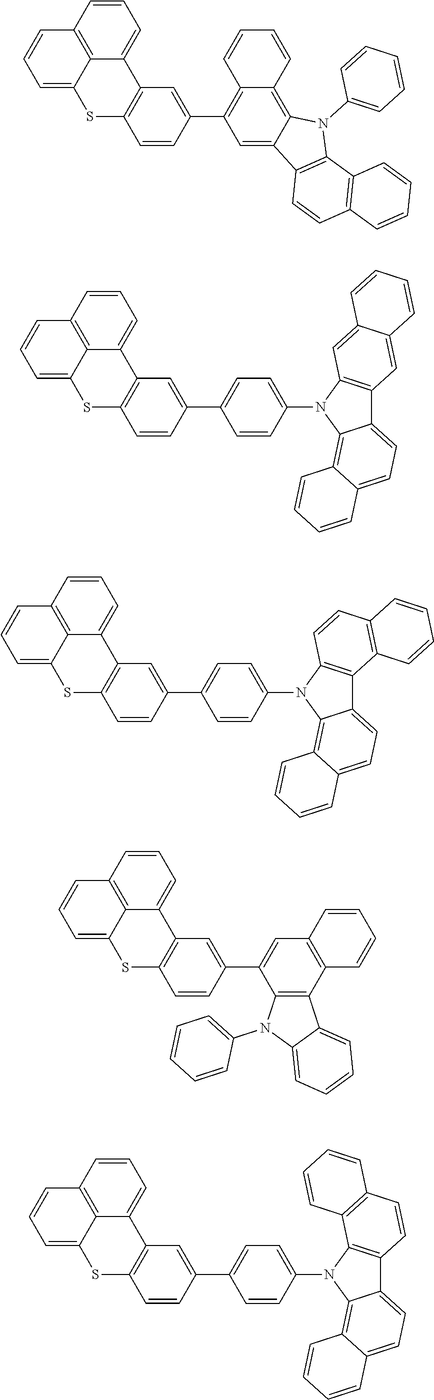

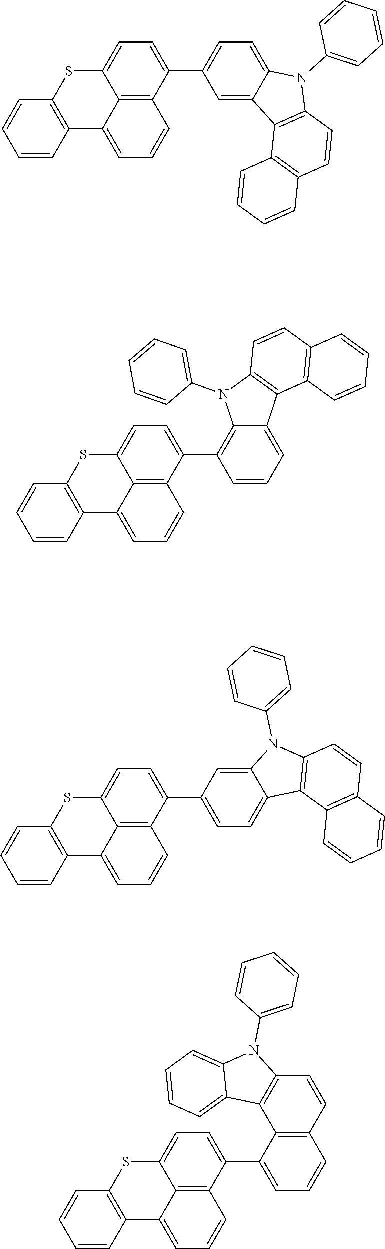 [SMILES: c1ccc(-n2c3ccc(-c4ccc5c6c(cccc46)-c4ccccc4S5)cc3c3c4ccccc4ccc32)cc1 ; c1ccc(-n2c3ccc4ccccc4c3c3cccc(-c4ccc5c6c(cccc46)-c4ccccc4S5)c32)cc1 ; c1ccc(-n2c3cc(-c4ccc5c6c(cccc46)-c4ccccc4S5)ccc3c3c4ccccc4ccc32)cc1 ; c1ccc(-n2c3ccccc3c3c4c(-c5ccc6c7c(cccc57)-c5ccccc5S6)cccc4ccc32)cc1]

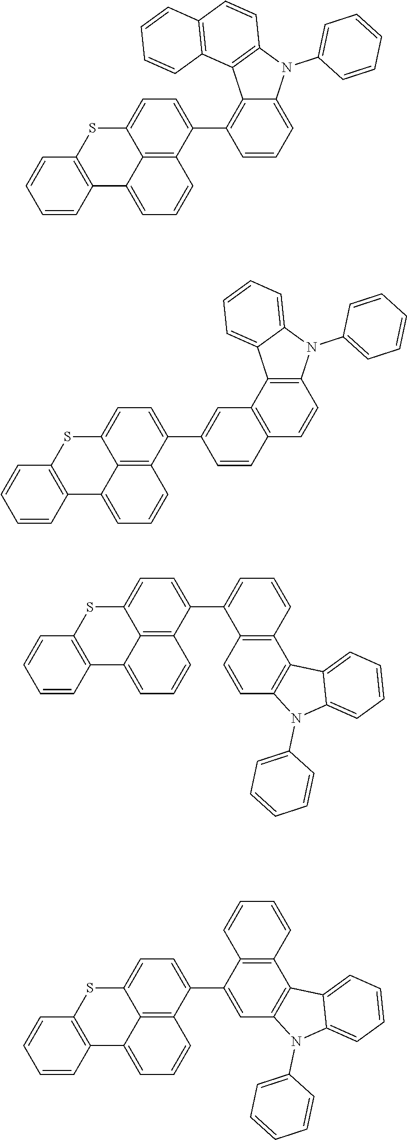 [SMILES: c1ccc(-n2c3cccc(-c4ccc5c6c(cccc46)-c4ccccc4S5)c3c3c4ccccc4ccc32)cc1 ; c1ccc(-n2c3ccccc3c3c4cc(-c5ccc6c7c(cccc57)-c5ccccc5S6)ccc4ccc32)cc1 ; c1ccc(-n2c3ccccc3c3c4cccc(-c5ccc6c7c(cccc57)-c5ccccc5S6)c4ccc32)cc1 ; c1ccc(-n2c3ccccc3c3c4ccccc4c(-c4ccc5c6c(cccc46)-c4ccccc4S5)cc32)cc1]

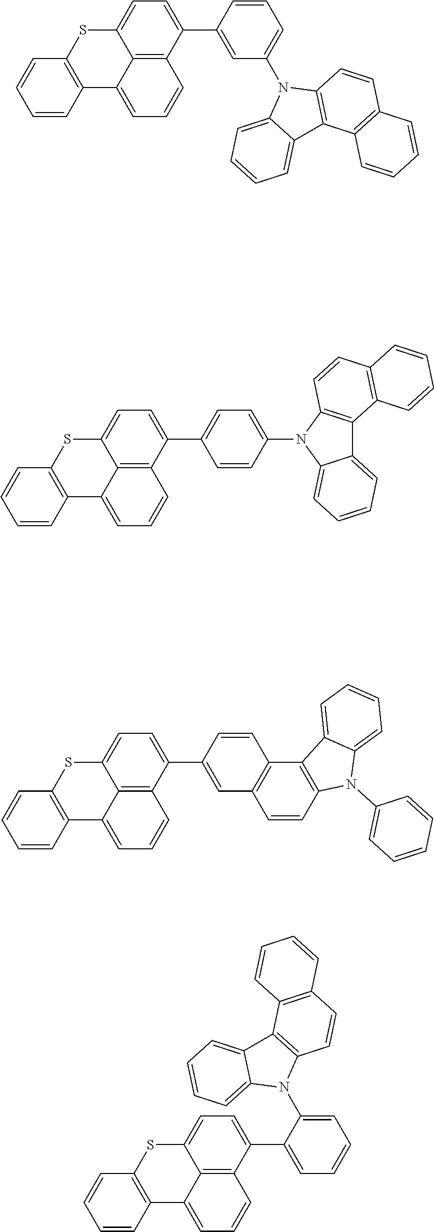 [SMILES: c1cc(-c2ccc3c4c(cccc24)-c2ccccc2S3)cc(-n2c3ccccc3c3c4ccccc4ccc32)c1 ; c1ccc2c(c1)Sc1ccc(-c3ccc(-n4c5ccccc5c5c6ccccc6ccc54)cc3)c3cccc-2c13 ; c1ccc(-n2c3ccccc3c3c4ccc(-c5ccc6c7c(cccc57)-c5ccccc5S6)cc4ccc32)cc1 ; c1ccc2c(c1)Sc1ccc(-c3ccccc3-n3c4ccccc4c4c5ccccc5ccc43)c3cccc-2c13]

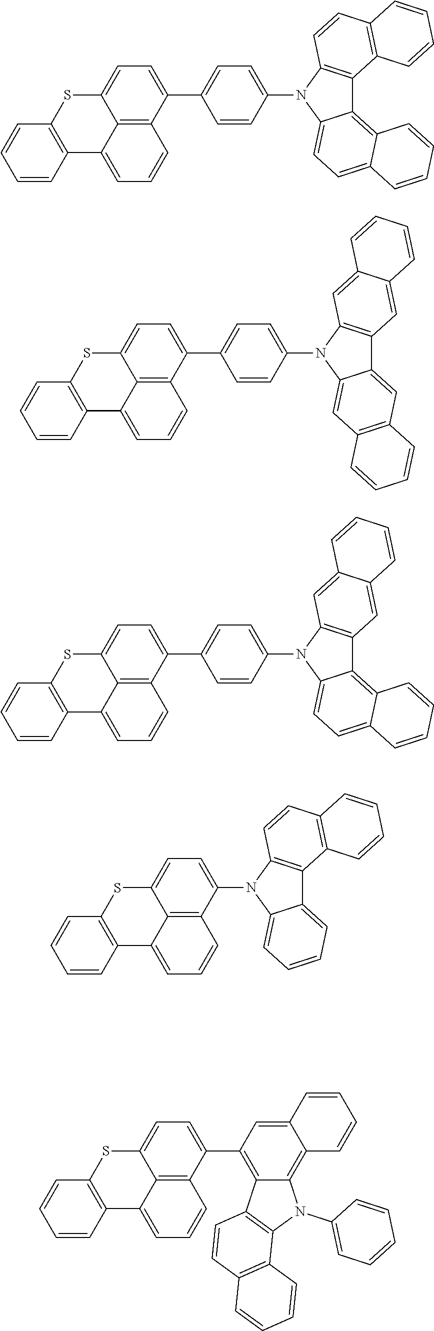

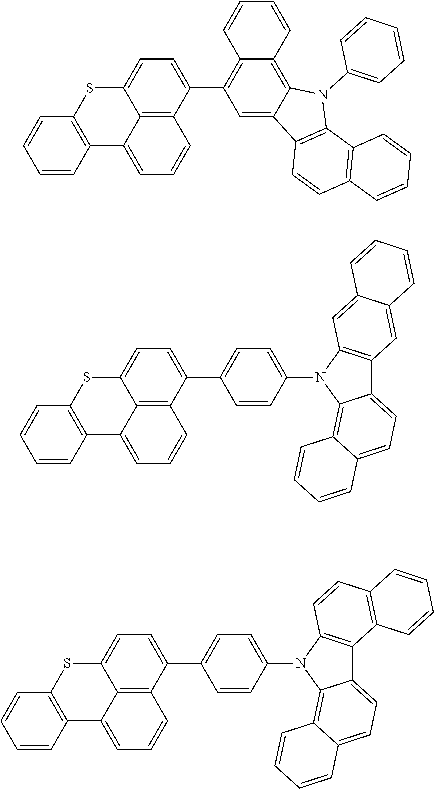

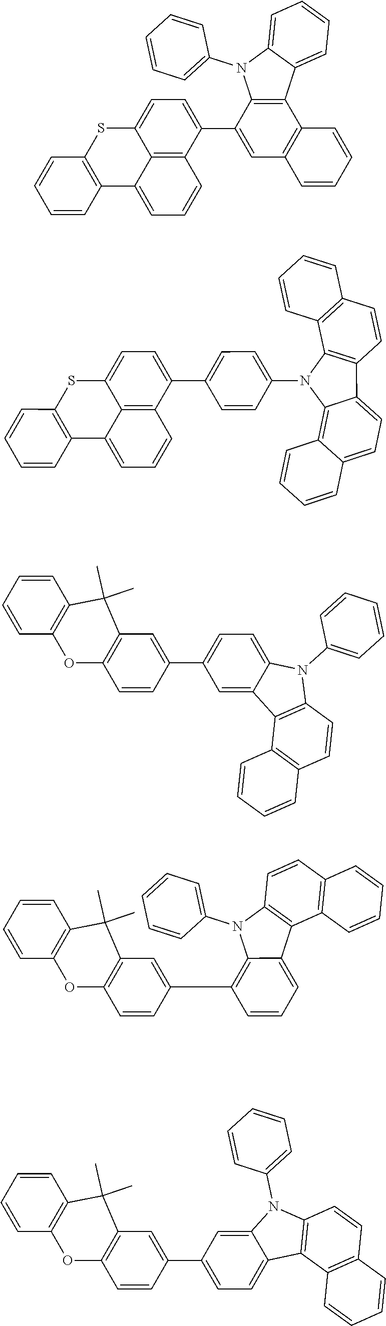 [SMILES: c1ccc(-n2c3ccccc3c3c4ccccc4cc(-c4ccc5c6c(cccc46)-c4ccccc4S5)c32)cc1 ; c1ccc2c(c1)Sc1ccc(-c3ccc(-n4c5c6ccccc6ccc5c5ccc6ccccc6c54)cc3)c3cccc-2c13 ; CC1(C)c2ccccc2Oc2ccc(-c3ccc4c(c3)c3c5ccccc5ccc3n4-c3ccccc3)cc21 ; CC1(C)c2ccccc2Oc2ccc(-c3cccc4c5c6ccccc6ccc5n(-c5ccccc5)c34)cc21 ; CC1(C)c2ccccc2Oc2ccc(-c3ccc4c5c6ccccc6ccc5n(-c5ccccc5)c4c3)cc21]

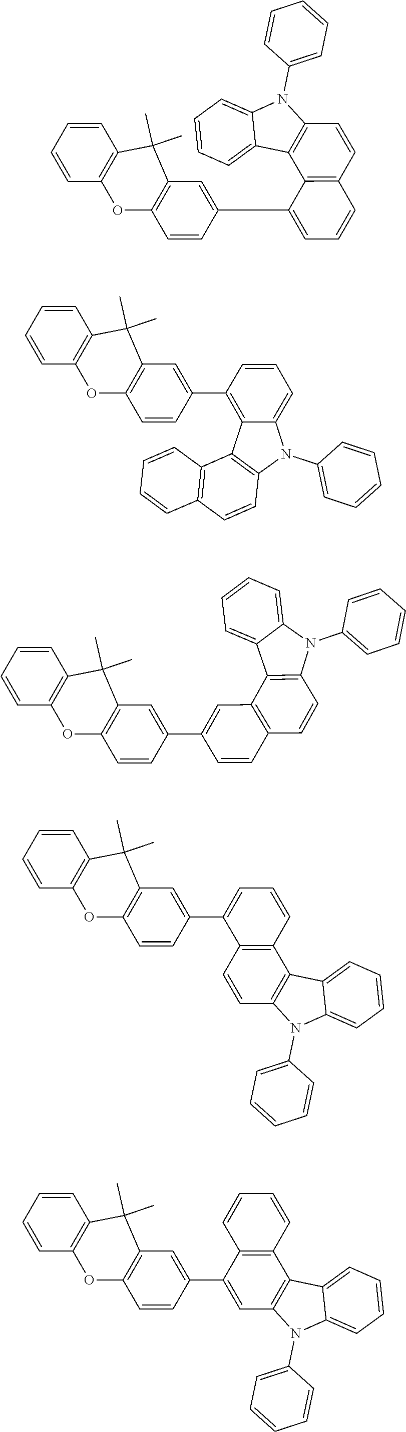 [SMILES: CC1(C)c2ccccc2Oc2ccc(-c3cccc4ccc5c(c6ccccc6n5-c5ccccc5)c34)cc21 ; CC1(C)c2ccccc2Oc2ccc(-c3cccc4c3c3c5ccccc5ccc3n4-c3ccccc3)cc21 ; CC1(C)c2ccccc2Oc2ccc(-c3ccc4ccc5c(c4c3)c3ccccc3n5-c3ccccc3)cc21 ; CC1(C)c2ccccc2Oc2ccc(-c3cccc4c3ccc3c4c4ccccc4n3-c3ccccc3)cc21 ; CC1(C)c2ccccc2Oc2ccc(-c3cc4c(c5ccccc35)c3ccccc3n4-c3ccccc3)cc21]

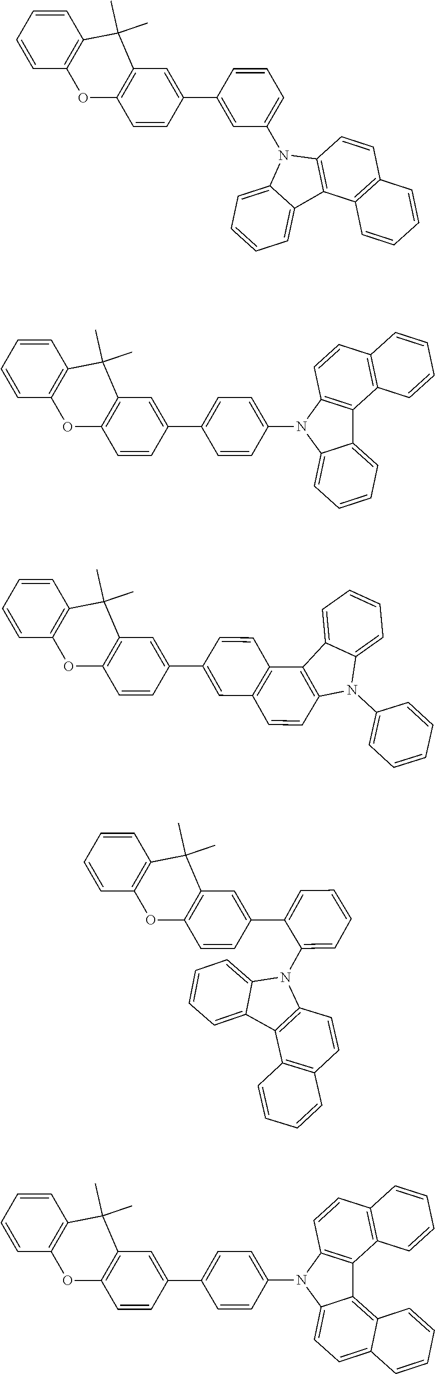 [SMILES: CC1(C)c2ccccc2Oc2ccc(-c3cccc(-n4c5ccccc5c5c6ccccc6ccc54)c3)cc21 ; CC1(C)c2ccccc2Oc2ccc(-c3ccc(-n4c5ccccc5c5c6ccccc6ccc54)cc3)cc21 ; CC1(C)c2ccccc2Oc2ccc(-c3ccc4c(ccc5c4c4ccccc4n5-c4ccccc4)c3)cc21 ; CC1(C)c2ccccc2Oc2ccc(-c3ccccc3-n3c4ccccc4c4c5ccccc5ccc43)cc21 ; CC1(C)c2ccccc2Oc2ccc(-c3ccc(-n4c5ccc6ccccc6c5c5c6ccccc6ccc54)cc3)cc21]

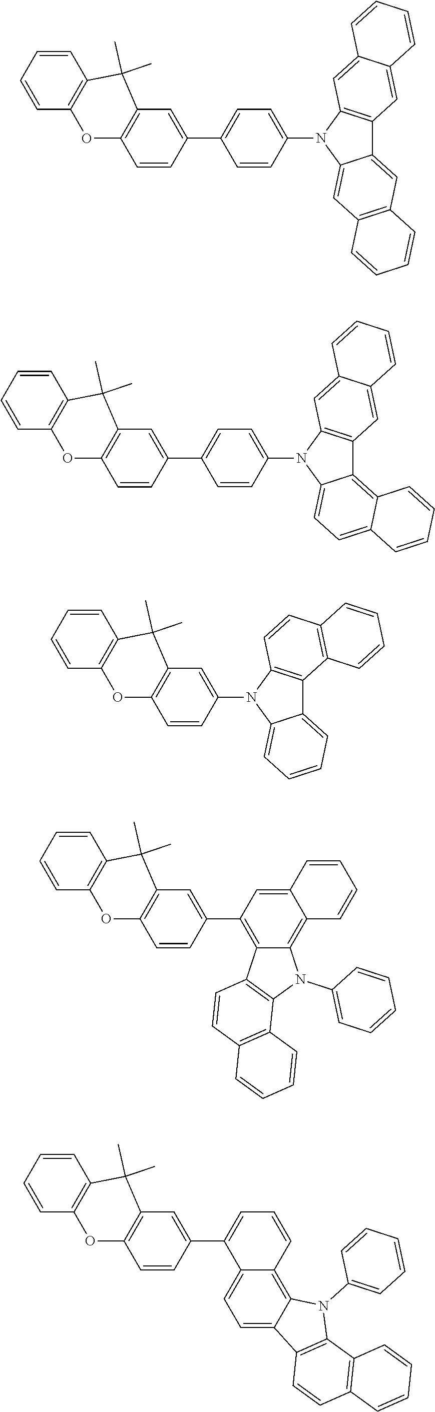 [SMILES: CC1(C)c2ccccc2Oc2ccc(-c3ccc(-n4c5cc6ccccc6cc5c5cc6ccccc6cc54)cc3)cc21 ; CC1(C)c2ccccc2Oc2ccc(-c3ccc(-n4c5cc6ccccc6cc5c5c6ccccc6ccc54)cc3)cc21 ; CC1(C)c2ccccc2Oc2ccc(-n3c4ccccc4c4c5ccccc5ccc43)cc21 ; CC1(C)c2ccccc2Oc2ccc(-c3cc4ccccc4c4c3c3ccc5ccccc5c3n4-c3ccccc3)cc21 ; CC1(C)c2ccccc2Oc2ccc(-c3cccc4c3ccc3c5ccc6ccccc6c5n(-c5ccccc5)c43)cc21]

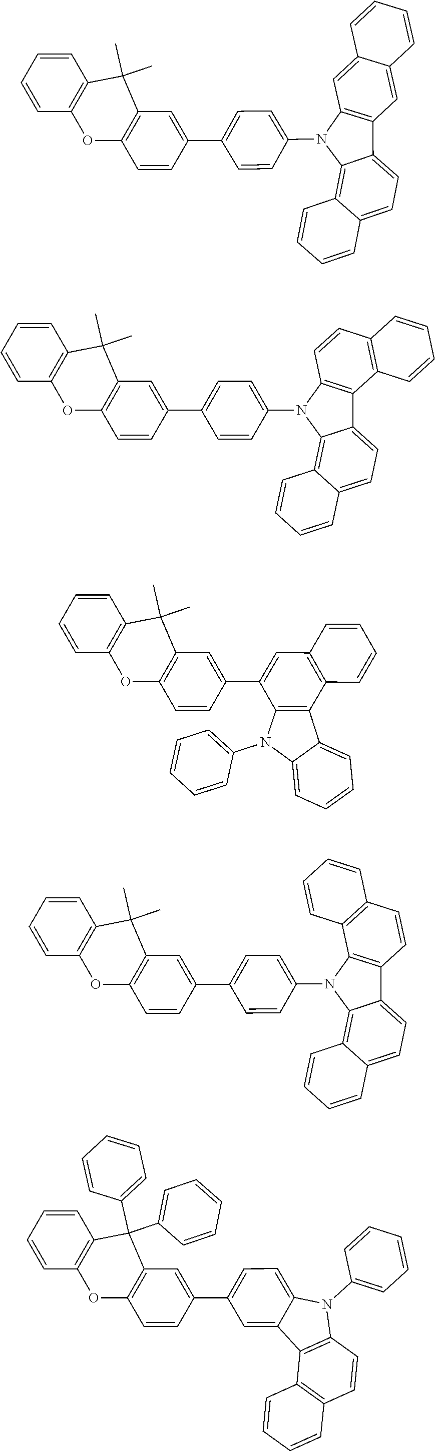 [SMILES: CC1(C)c2ccccc2Oc2ccc(-c3ccc(-n4c5cc6ccccc6cc5c5ccc6ccccc6c54)cc3)cc21 ; CC1(C)c2ccccc2Oc2ccc(-c3ccc(-n4c5ccc6ccccc6c5c5ccc6ccccc6c54)cc3)cc21 ; CC1(C)c2ccccc2Oc2ccc(-c3cc4ccccc4c4c5ccccc5n(-c5ccccc5)c34)cc21 ; CC1(C)c2ccccc2Oc2ccc(-c3ccc(-n4c5c6ccccc6ccc5c5ccc6ccccc6c54)cc3)cc21 ; c1ccc(-n2c3ccc(-c4ccc5c(c4)C(c4ccccc4)(c4ccccc4)c4ccccc4O5)cc3c3c4ccccc4ccc32)cc1]

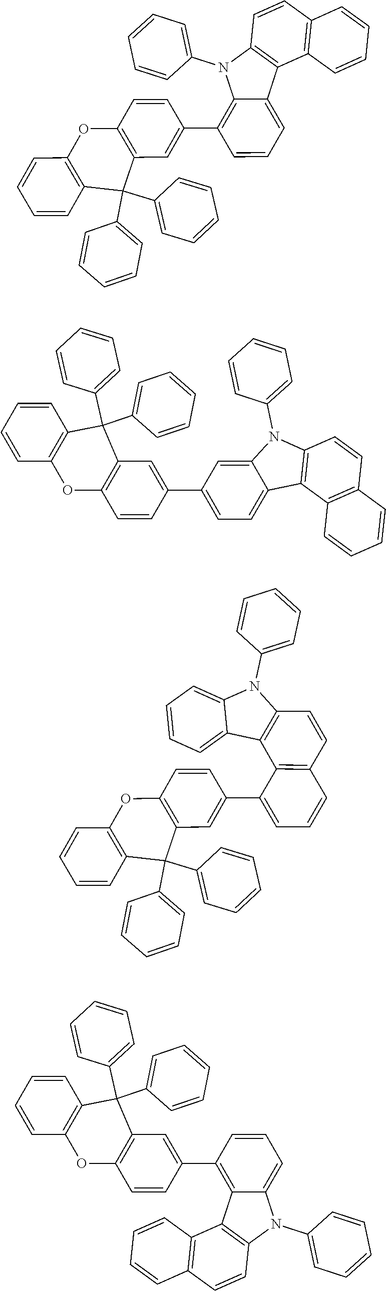 [SMILES: c1ccc(-n2c3ccc4ccccc4c3c3cccc(-c4ccc5c(c4)C(c4ccccc4)(c4ccccc4)c4ccccc4O5)c32)cc1 ; c1ccc(-n2c3cc(-c4ccc5c(c4)C(c4ccccc4)(c4ccccc4)c4ccccc4O5)ccc3c3c4ccccc4ccc32)cc1 ; c1ccc(-n2c3ccccc3c3c4c(-c5ccc6c(c5)C(c5ccccc5)(c5ccccc5)c5ccccc5O6)cccc4ccc32)cc1 ; c1ccc(-n2c3cccc(-c4ccc5c(c4)C(c4ccccc4)(c4ccccc4)c4ccccc4O5)c3c3c4ccccc4ccc32)cc1]

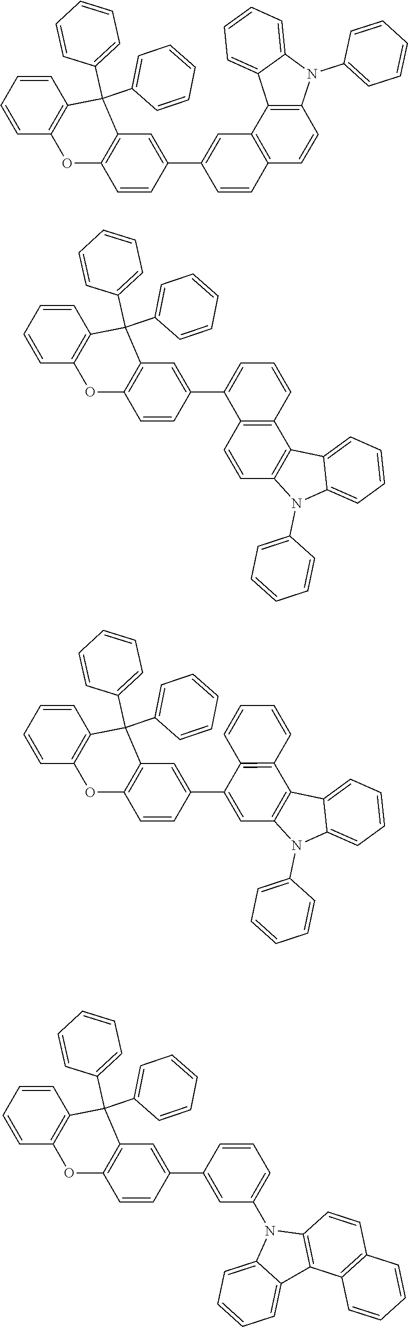

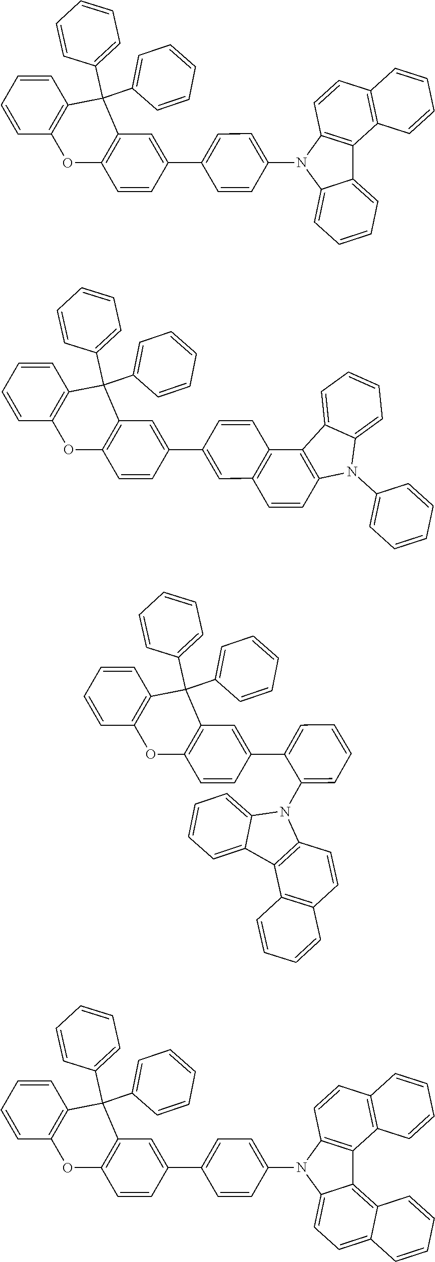 [SMILES: c1ccc(C2(c3ccccc3)c3ccccc3Oc3ccc(-c4ccc(-n5c6ccccc6c6c7ccccc7ccc65)cc4)cc32)cc1 ; c1ccc(-n2c3ccccc3c3c4ccc(-c5ccc6c(c5)C(c5ccccc5)(c5ccccc5)c5ccccc5O6)cc4ccc32)cc1 ; c1ccc(C2(c3ccccc3)c3ccccc3Oc3ccc(-c4ccccc4-n4c5ccccc5c5c6ccccc6ccc54)cc32)cc1 ; c1ccc(C2(c3ccccc3)c3ccccc3Oc3ccc(-c4ccc(-n5c6ccc7ccccc7c6c6c7ccccc7ccc65)cc4)cc32)cc1]

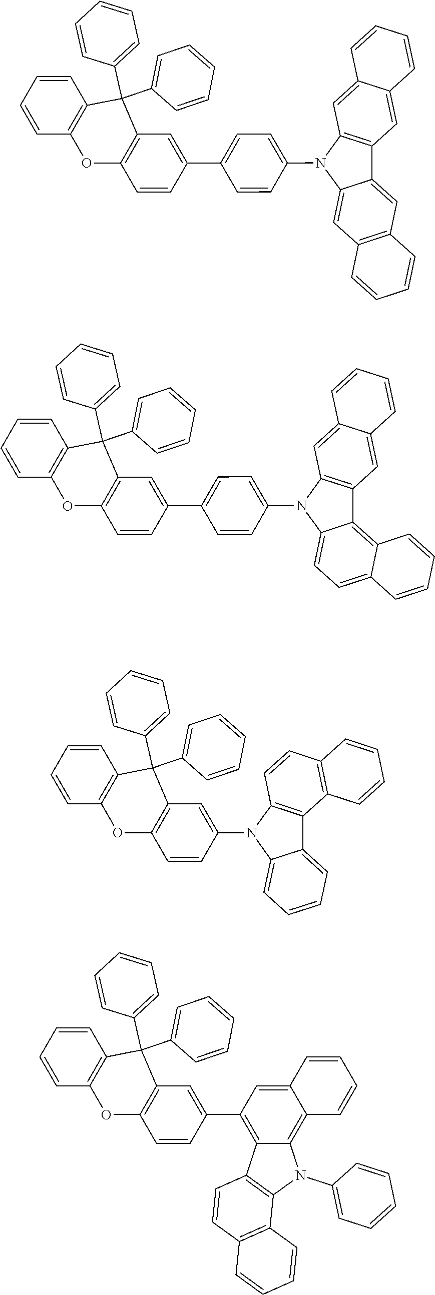 [SMILES: c1ccc(C2(c3ccccc3)c3ccccc3Oc3ccc(-c4ccc(-n5c6cc7ccccc7cc6c6cc7ccccc7cc65)cc4)cc32)cc1 ; c1ccc(C2(c3ccccc3)c3ccccc3Oc3ccc(-c4ccc(-n5c6cc7ccccc7cc6c6c7ccccc7ccc65)cc4)cc32)cc1 ; c1ccc(C2(c3ccccc3)c3ccccc3Oc3ccc(-n4c5ccccc5c5c6ccccc6ccc54)cc32)cc1 ; c1ccc(-n2c3c4ccccc4ccc3c3c(-c4ccc5c(c4)C(c4ccccc4)(c4ccccc4)c4ccccc4O5)cc4ccccc4c32)cc1]

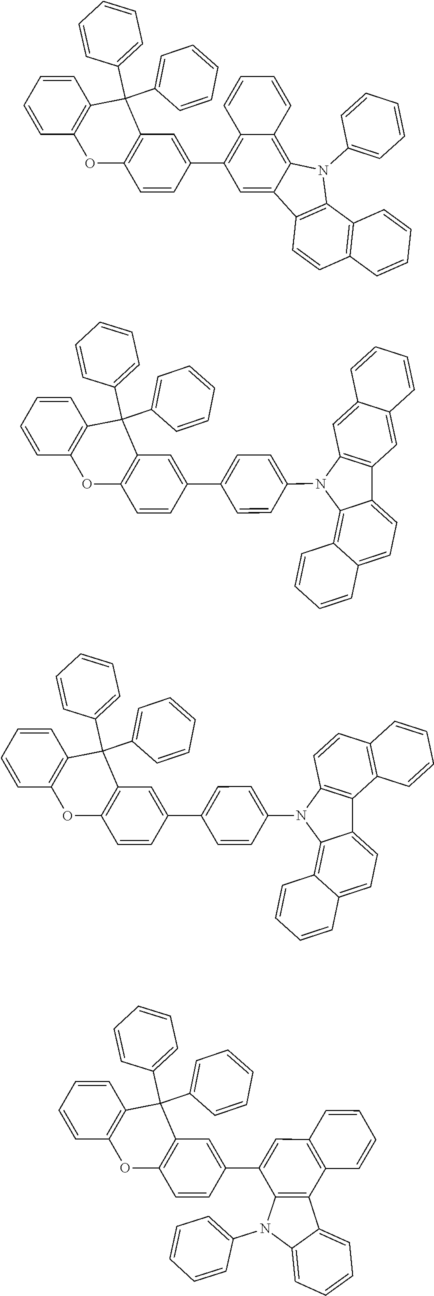 [SMILES: c1ccc(-n2c3c4ccccc4ccc3c3cc(-c4ccc5c(c4)C(c4ccccc4)(c4ccccc4)c4ccccc4O5)c4ccccc4c32)cc1 ; c1ccc(C2(c3ccccc3)c3ccccc3Oc3ccc(-c4ccc(-n5c6cc7ccccc7cc6c6ccc7ccccc7c65)cc4)cc32)cc1 ; c1ccc(C2(c3ccccc3)c3ccccc3Oc3ccc(-c4ccc(-n5c6ccc7ccccc7c6c6ccc7ccccc7c65)cc4)cc32)cc1 ; c1ccc(-n2c3ccccc3c3c4ccccc4cc(-c4ccc5c(c4)C(c4ccccc4)(c4ccccc4)c4ccccc4O5)c32)cc1]

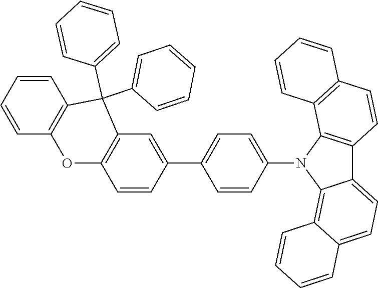 [SMILES: c1ccc(C2(c3ccccc3)c3ccccc3Oc3ccc(-c4ccc(-n5c6c7ccccc7ccc6c6ccc7ccccc7c65)cc4)cc32)cc1]

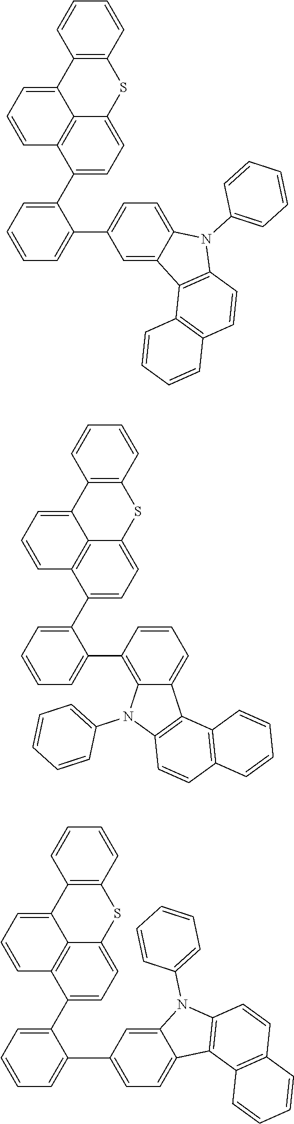 [SMILES: c1ccc(-n2c3ccc(-c4ccccc4-c4ccc5c6c(cccc46)-c4ccccc4S5)cc3c3c4ccccc4ccc32)cc1 ; c1ccc(-n2c3ccc4ccccc4c3c3cccc(-c4ccccc4-c4ccc5c6c(cccc46)-c4ccccc4S5)c32)cc1 ; c1ccc(-n2c3cc(-c4ccccc4-c4ccc5c6c(cccc46)-c4ccccc4S5)ccc3c3c4ccccc4ccc32)cc1]

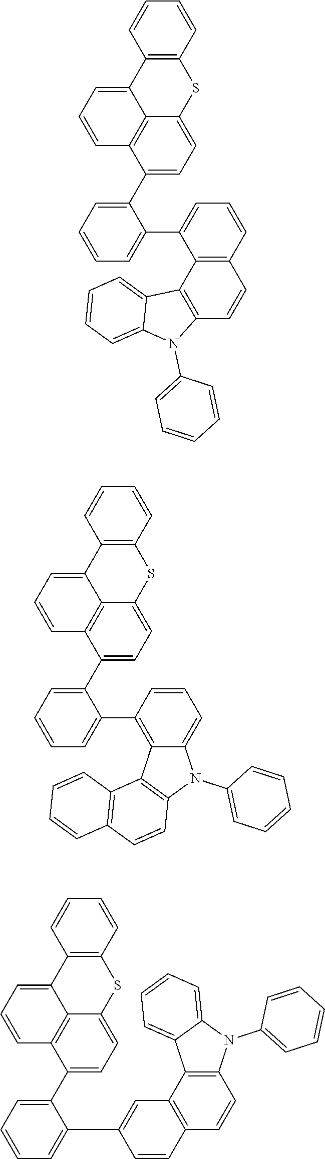 [SMILES: c1ccc(-n2c3ccccc3c3c4c(-c5ccccc5-c5ccc6c7c(cccc57)-c5ccccc5S6)cccc4ccc32)cc1 ; c1ccc(-n2c3cccc(-c4ccccc4-c4ccc5c6c(cccc46)-c4ccccc4S5)c3c3c4ccccc4ccc32)cc1 ; c1ccc(-n2c3ccccc3c3c4cc(-c5ccccc5-c5ccc6c7c(cccc57)-c5ccccc5S6)ccc4ccc32)cc1]

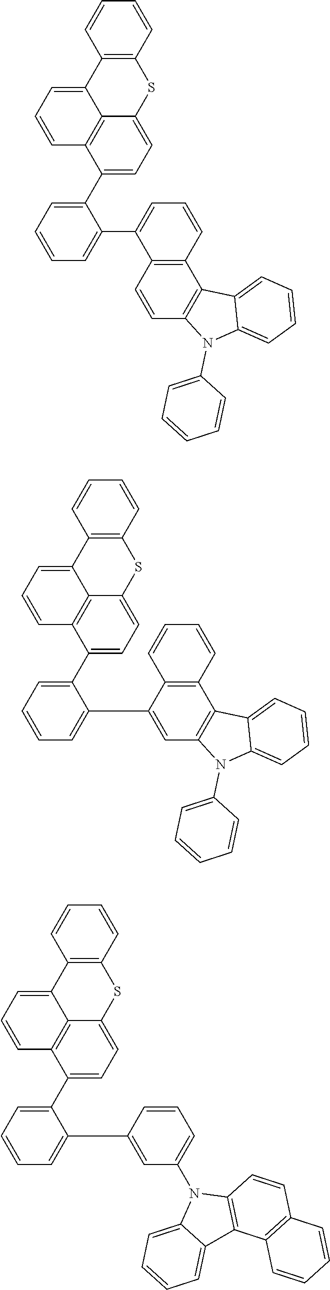 [SMILES: c1ccc(-n2c3ccccc3c3c4cccc(-c5ccccc5-c5ccc6c7c(cccc57)-c5ccccc5S6)c4ccc32)cc1 ; c1ccc(-n2c3ccccc3c3c4ccccc4c(-c4ccccc4-c4ccc5c6c(cccc46)-c4ccccc4S5)cc32)cc1 ; c1cc(-c2ccccc2-c2ccc3c4c(cccc24)-c2ccccc2S3)cc(-n2c3ccccc3c3c4ccccc4ccc32)c1]

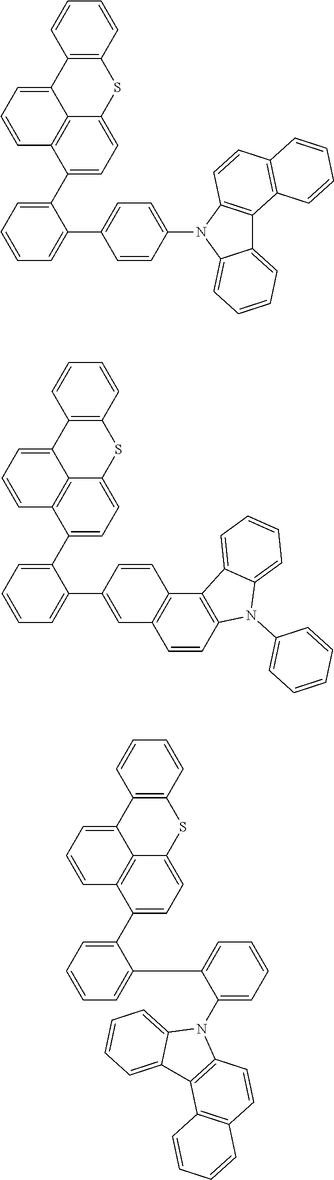 [SMILES: c1ccc2c(c1)Sc1ccc(-c3ccccc3-c3ccc(-n4c5ccccc5c5c6ccccc6ccc54)cc3)c3cccc-2c13 ; c1ccc(-n2c3ccccc3c3c4ccc(-c5ccccc5-c5ccc6c7c(cccc57)-c5ccccc5S6)cc4ccc32)cc1 ; c1ccc2c(c1)Sc1ccc(-c3ccccc3-c3ccccc3-n3c4ccccc4c4c5ccccc5ccc43)c3cccc-2c13]

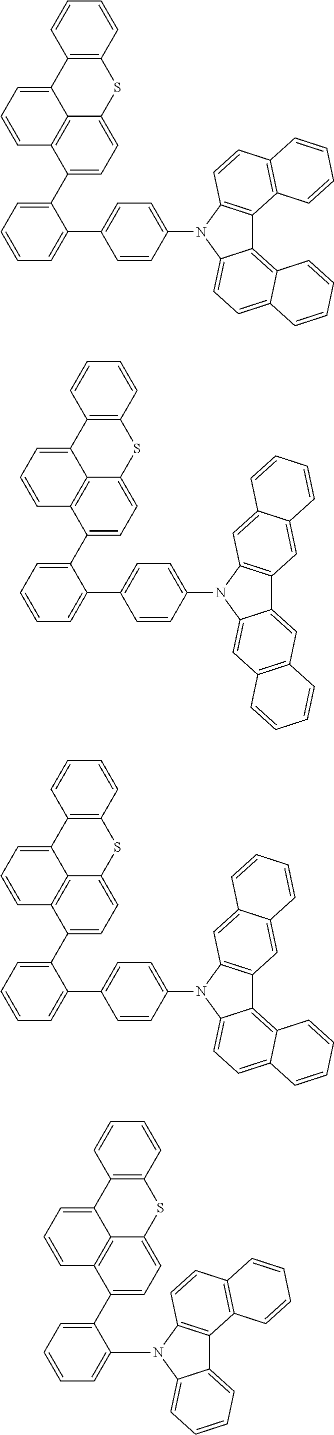 [SMILES: c1ccc2c(c1)Sc1ccc(-c3ccccc3-c3ccc(-n4c5ccc6ccccc6c5c5c6ccccc6ccc54)cc3)c3cccc-2c13 ; c1ccc2c(c1)Sc1ccc(-c3ccccc3-c3ccc(-n4c5cc6ccccc6cc5c5cc6ccccc6cc54)cc3)c3cccc-2c13 ; c1ccc2c(c1)Sc1ccc(-c3ccccc3-c3ccc(-n4c5cc6ccccc6cc5c5c6ccccc6ccc54)cc3)c3cccc-2c13 ; c1ccc2c(c1)Sc1ccc(-c3ccccc3-n3c4ccccc4c4c5ccccc5ccc43)c3cccc-2c13]

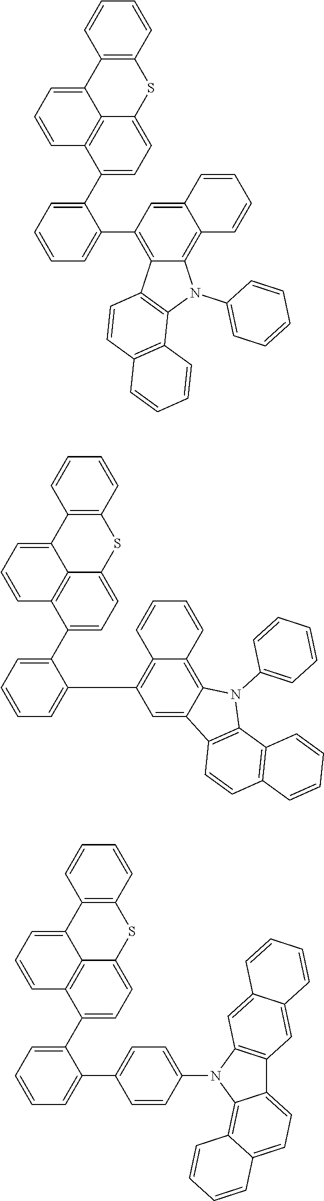 [SMILES: c1ccc(-n2c3c4ccccc4ccc3c3c(-c4ccccc4-c4ccc5c6c(cccc46)-c4ccccc4S5)cc4ccccc4c32)cc1 ; c1ccc(-n2c3c4ccccc4ccc3c3cc(-c4ccccc4-c4ccc5c6c(cccc46)-c4ccccc4S5)c4ccccc4c32)cc1 ; c1ccc2c(c1)Sc1ccc(-c3ccccc3-c3ccc(-n4c5cc6ccccc6cc5c5ccc6ccccc6c54)cc3)c3cccc-2c13]

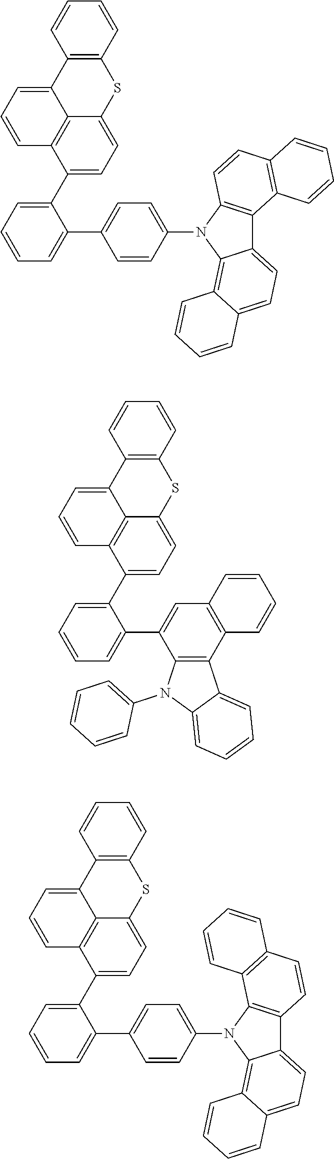 [SMILES: c1ccc2c(c1)Sc1ccc(-c3ccccc3-c3ccc(-n4c5ccc6ccccc6c5c5ccc6ccccc6c54)cc3)c3cccc-2c13 ; c1ccc(-n2c3ccccc3c3c4ccccc4cc(-c4ccccc4-c4ccc5c6c(cccc46)-c4ccccc4S5)c32)cc1 ; c1ccc2c(c1)Sc1ccc(-c3ccccc3-c3ccc(-n4c5c6ccccc6ccc5c5ccc6ccccc6c54)cc3)c3cccc-2c13]

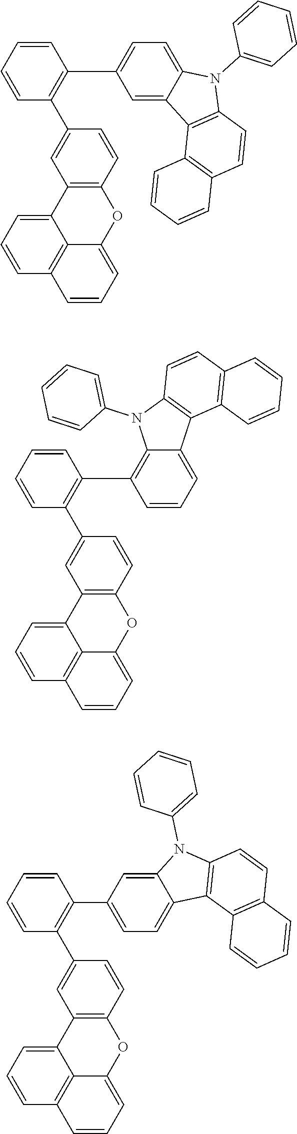 [SMILES: c1ccc(-n2c3ccc(-c4ccccc4-c4ccc5c(c4)-c4cccc6cccc(c46)O5)cc3c3c4ccccc4ccc32)cc1 ; c1ccc(-n2c3ccc4ccccc4c3c3cccc(-c4ccccc4-c4ccc5c(c4)-c4cccc6cccc(c46)O5)c32)cc1 ; c1ccc(-n2c3cc(-c4ccccc4-c4ccc5c(c4)-c4cccc6cccc(c46)O5)ccc3c3c4ccccc4ccc32)cc1]

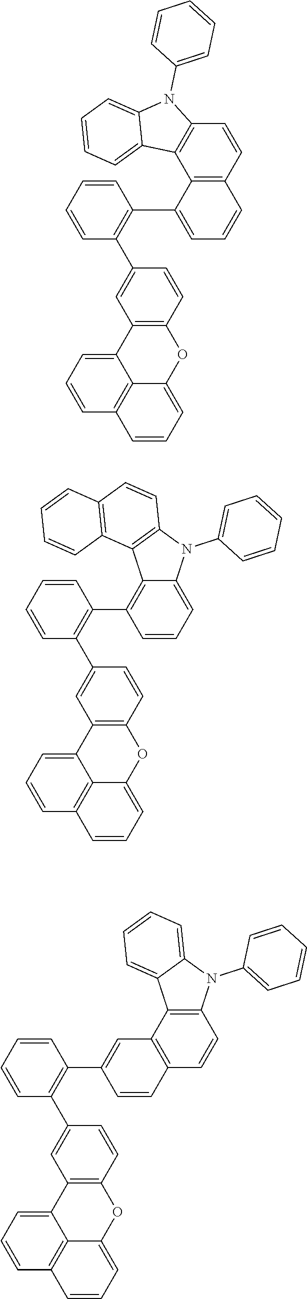

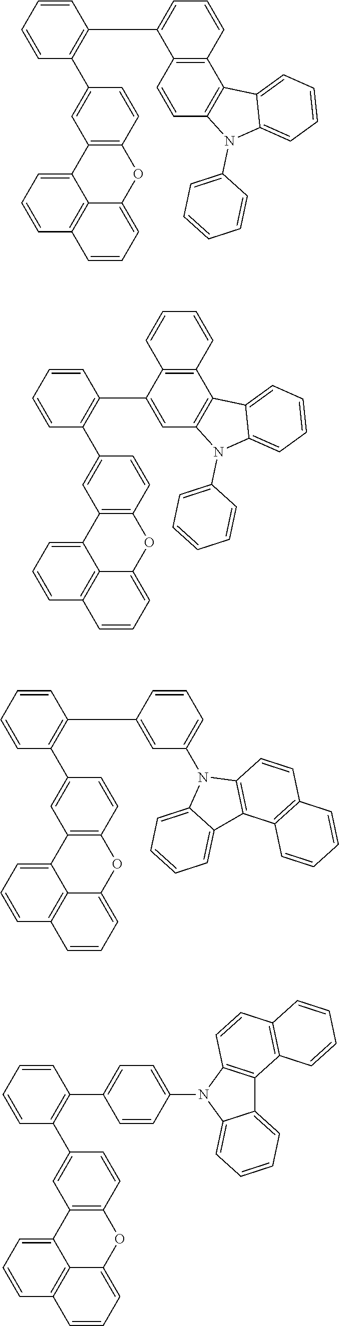 [SMILES: c1ccc(-n2c3ccccc3c3c4cccc(-c5ccccc5-c5ccc6c(c5)-c5cccc7cccc(c57)O6)c4ccc32)cc1 ; c1ccc(-n2c3ccccc3c3c4ccccc4c(-c4ccccc4-c4ccc5c(c4)-c4cccc6cccc(c46)O5)cc32)cc1 ; c1cc(-c2ccccc2-c2ccc3c(c2)-c2cccc4cccc(c24)O3)cc(-n2c3ccccc3c3c4ccccc4ccc32)c1 ; c1ccc(-c2ccc3c(c2)-c2cccc4cccc(c24)O3)c(-c2ccc(-n3c4ccccc4c4c5ccccc5ccc43)cc2)c1]

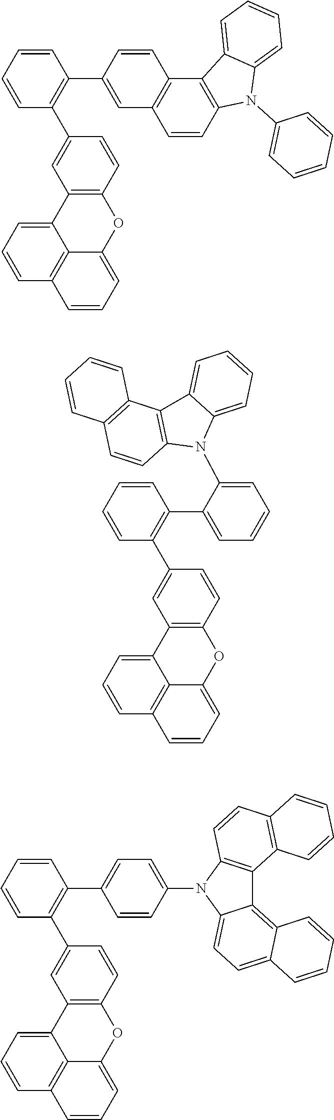 [SMILES: c1ccc(-n2c3ccccc3c3c4ccc(-c5ccccc5-c5ccc6c(c5)-c5cccc7cccc(c57)O6)cc4ccc32)cc1 ; c1ccc(-c2ccccc2-n2c3ccccc3c3c4ccccc4ccc32)c(-c2ccc3c(c2)-c2cccc4cccc(c24)O3)c1 ; c1ccc(-c2ccc3c(c2)-c2cccc4cccc(c24)O3)c(-c2ccc(-n3c4ccc5ccccc5c4c4c5ccccc5ccc43)cc2)c1]

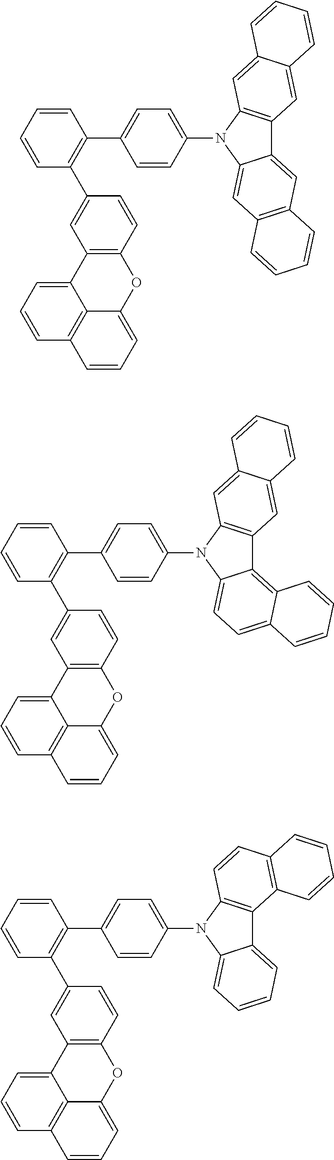 [SMILES: c1ccc(-c2ccc3c(c2)-c2cccc4cccc(c24)O3)c(-c2ccc(-n3c4cc5ccccc5cc4c4cc5ccccc5cc43)cc2)c1 ; c1ccc(-c2ccc3c(c2)-c2cccc4cccc(c24)O3)c(-c2ccc(-n3c4cc5ccccc5cc4c4c5ccccc5ccc43)cc2)c1 ; c1ccc(-c2ccc3c(c2)-c2cccc4cccc(c24)O3)c(-c2ccc(-n3c4ccccc4c4c5ccccc5ccc43)cc2)c1]

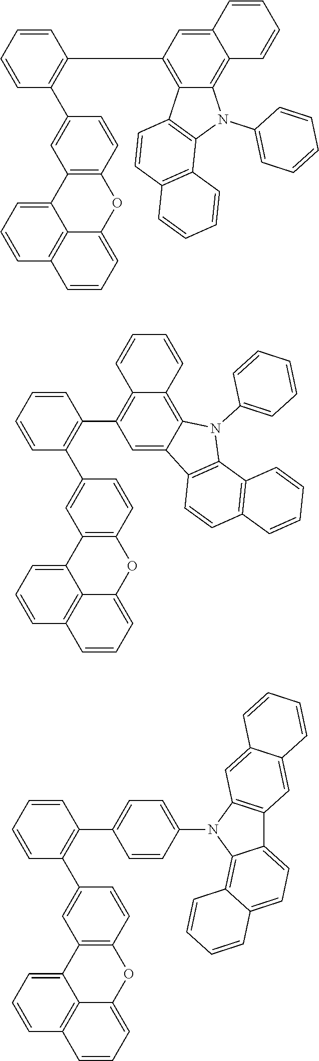 [SMILES: c1ccc(-n2c3c4ccccc4ccc3c3c(-c4ccccc4-c4ccc5c(c4)-c4cccc6cccc(c46)O5)cc4ccccc4c32)cc1 ; c1ccc(-n2c3c4ccccc4ccc3c3cc(-c4ccccc4-c4ccc5c(c4)-c4cccc6cccc(c46)O5)c4ccccc4c32)cc1 ; c1ccc(-c2ccc3c(c2)-c2cccc4cccc(c24)O3)c(-c2ccc(-n3c4cc5ccccc5cc4c4ccc5ccccc5c43)cc2)c1]

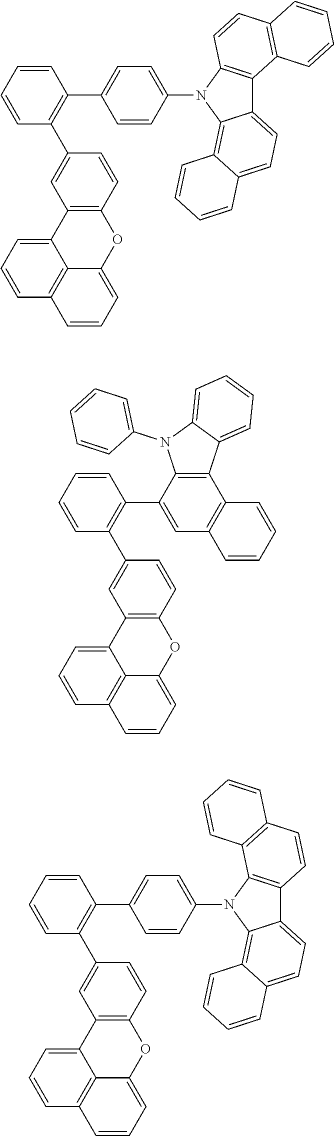 [SMILES: c1ccc(-c2ccc3c(c2)-c2cccc4cccc(c24)O3)c(-c2ccc(-n3c4ccc5ccccc5c4c4ccc5ccccc5c43)cc2)c1 ; c1ccc(-n2c3ccccc3c3c4ccccc4cc(-c4ccccc4-c4ccc5c(c4)-c4cccc6cccc(c46)O5)c32)cc1 ; c1ccc(-c2ccc3c(c2)-c2cccc4cccc(c24)O3)c(-c2ccc(-n3c4c5ccccc5ccc4c4ccc5ccccc5c43)cc2)c1]

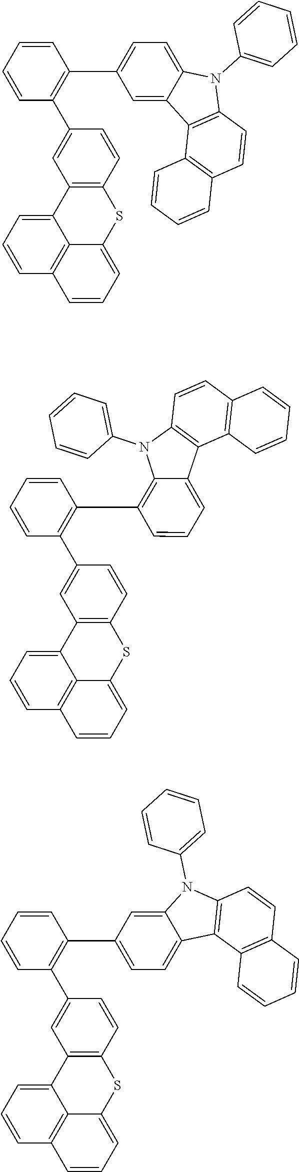 [SMILES: c1ccc(-n2c3ccc(-c4ccccc4-c4ccc5c(c4)-c4cccc6cccc(c46)S5)cc3c3c4ccccc4ccc32)cc1 ; c1ccc(-n2c3ccc4ccccc4c3c3cccc(-c4ccccc4-c4ccc5c(c4)-c4cccc6cccc(c46)S5)c32)cc1 ; c1ccc(-n2c3cc(-c4ccccc4-c4ccc5c(c4)-c4cccc6cccc(c46)S5)ccc3c3c4ccccc4ccc32)cc1]

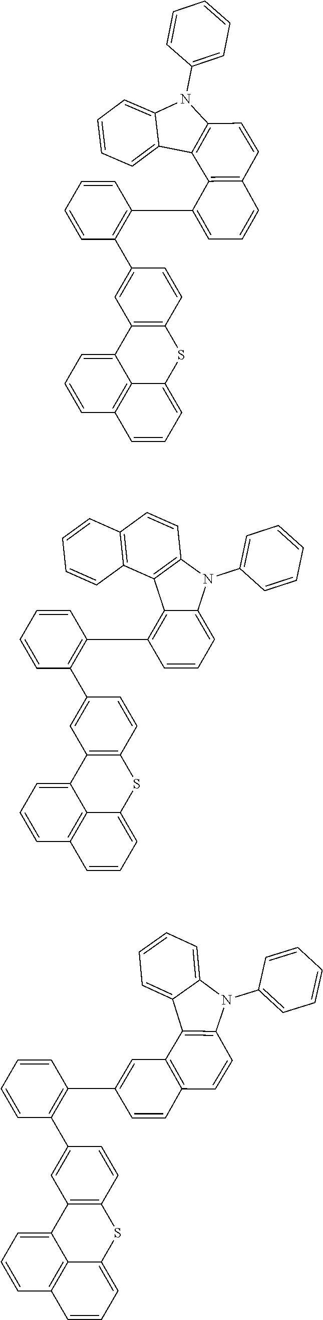 [SMILES: c1ccc(-n2c3ccccc3c3c4c(-c5ccccc5-c5ccc6c(c5)-c5cccc7cccc(c57)S6)cccc4ccc32)cc1 ; c1ccc(-n2c3cccc(-c4ccccc4-c4ccc5c(c4)-c4cccc6cccc(c46)S5)c3c3c4ccccc4ccc32)cc1 ; c1ccc(-n2c3ccccc3c3c4cc(-c5ccccc5-c5ccc6c(c5)-c5cccc7cccc(c57)S6)ccc4ccc32)cc1]

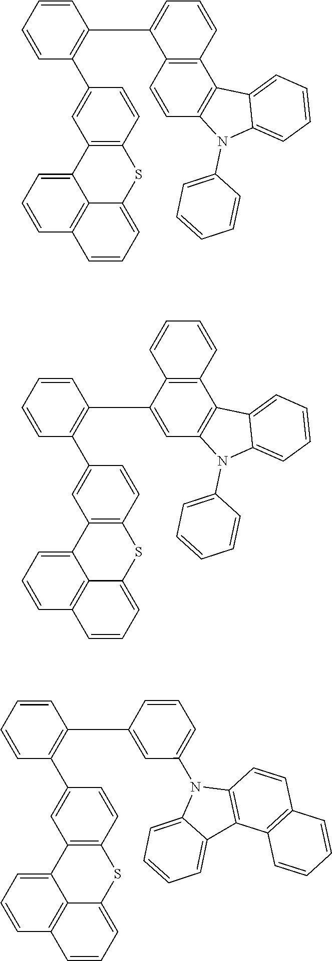 [SMILES: c1ccc(-n2c3ccccc3c3c4cccc(-c5ccccc5-c5ccc6c(c5)-c5cccc7cccc(c57)S6)c4ccc32)cc1 ; c1ccc(-n2c3ccccc3c3c4ccccc4c(-c4ccccc4-c4ccc5c(c4)-c4cccc6cccc(c46)S5)cc32)cc1 ; c1cc(-c2ccccc2-c2ccc3c(c2)-c2cccc4cccc(c24)S3)cc(-n2c3ccccc3c3c4ccccc4ccc32)c1]

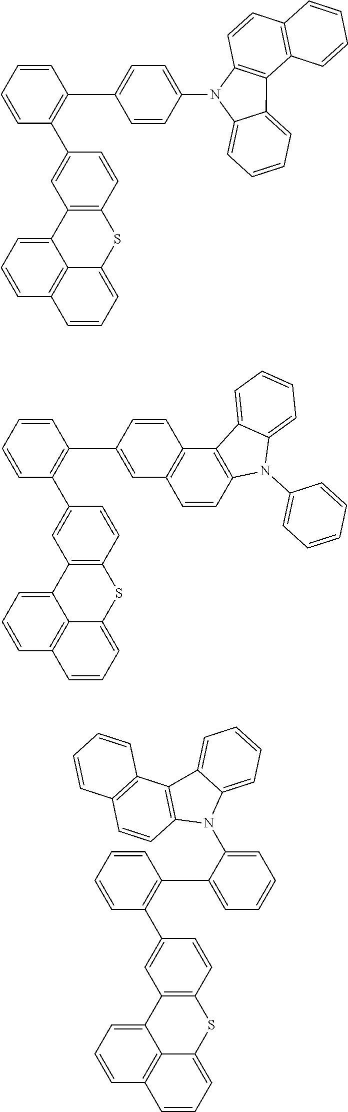 [SMILES: c1ccc(-c2ccc3c(c2)-c2cccc4cccc(c24)S3)c(-c2ccc(-n3c4ccccc4c4c5ccccc5ccc43)cc2)c1 ; c1ccc(-n2c3ccccc3c3c4ccc(-c5ccccc5-c5ccc6c(c5)-c5cccc7cccc(c57)S6)cc4ccc32)cc1 ; c1ccc(-c2ccccc2-n2c3ccccc3c3c4ccccc4ccc32)c(-c2ccc3c(c2)-c2cccc4cccc(c24)S3)c1]

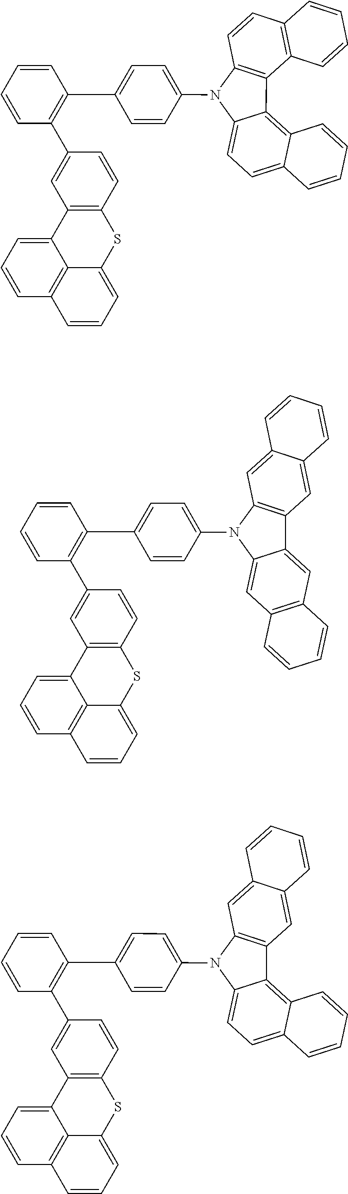 [SMILES: c1ccc(-c2ccc3c(c2)-c2cccc4cccc(c24)S3)c(-c2ccc(-n3c4ccc5ccccc5c4c4c5ccccc5ccc43)cc2)c1 ; c1ccc(-c2ccc3c(c2)-c2cccc4cccc(c24)S3)c(-c2ccc(-n3c4cc5ccccc5cc4c4cc5ccccc5cc43)cc2)c1 ; c1ccc(-c2ccc3c(c2)-c2cccc4cccc(c24)S3)c(-c2ccc(-n3c4cc5ccccc5cc4c4c5ccccc5ccc43)cc2)c1]

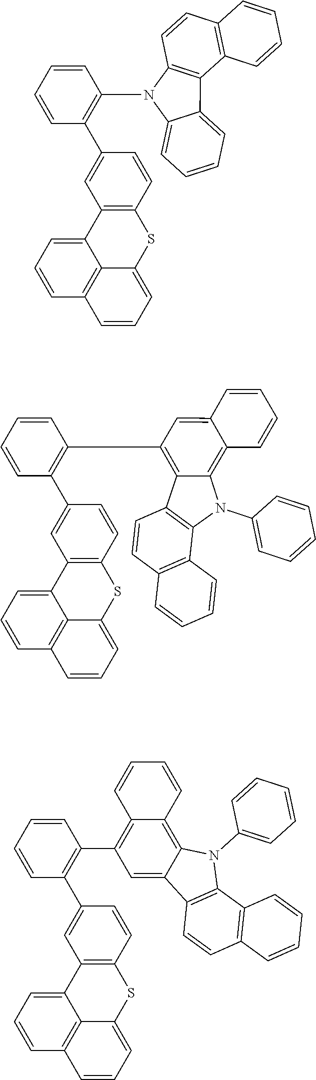 [SMILES: c1ccc(-n2c3ccccc3c3c4ccccc4ccc32)c(-c2ccc3c(c2)-c2cccc4cccc(c24)S3)c1 ; c1ccc(-n2c3c4ccccc4ccc3c3c(-c4ccccc4-c4ccc5c(c4)-c4cccc6cccc(c46)S5)cc4ccccc4c32)cc1 ; c1ccc(-n2c3c4ccccc4ccc3c3cc(-c4ccccc4-c4ccc5c(c4)-c4cccc6cccc(c46)S5)c4ccccc4c32)cc1]

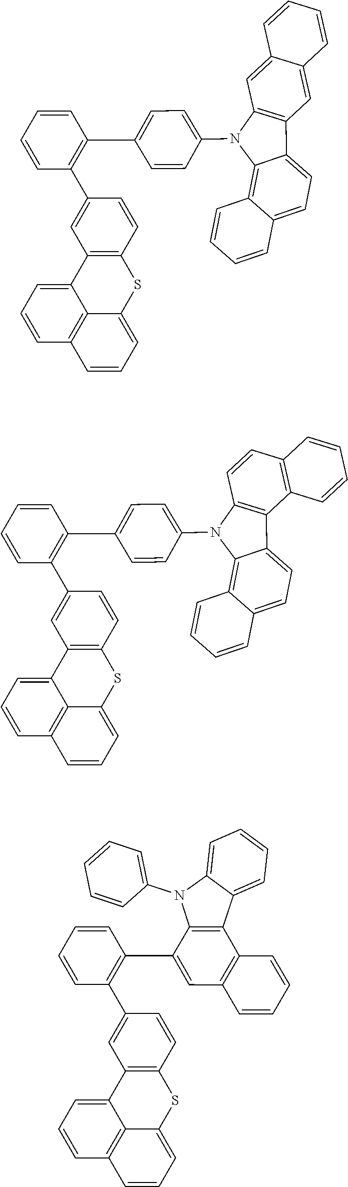 [SMILES: c1ccc(-c2ccc3c(c2)-c2cccc4cccc(c24)S3)c(-c2ccc(-n3c4cc5ccccc5cc4c4ccc5ccccc5c43)cc2)c1 ; c1ccc(-c2ccc3c(c2)-c2cccc4cccc(c24)S3)c(-c2ccc(-n3c4ccc5ccccc5c4c4ccc5ccccc5c43)cc2)c1 ; c1ccc(-n2c3ccccc3c3c4ccccc4cc(-c4ccccc4-c4ccc5c(c4)-c4cccc6cccc(c46)S5)c32)cc1]

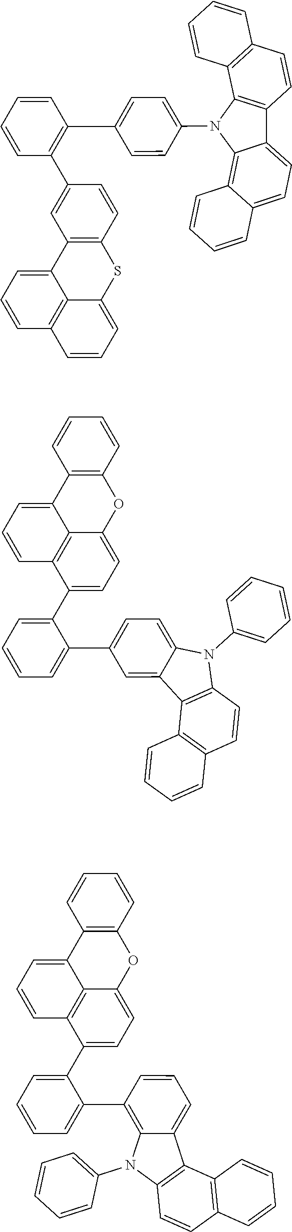 [SMILES: c1ccc(-c2ccc3c(c2)-c2cccc4cccc(c24)S3)c(-c2ccc(-n3c4c5ccccc5ccc4c4ccc5ccccc5c43)cc2)c1 ; c1ccc(-n2c3ccc(-c4ccccc4-c4ccc5c6c(cccc46)-c4ccccc4O5)cc3c3c4ccccc4ccc32)cc1 ; c1ccc(-n2c3ccc4ccccc4c3c3cccc(-c4ccccc4-c4ccc5c6c(cccc46)-c4ccccc4O5)c32)cc1]

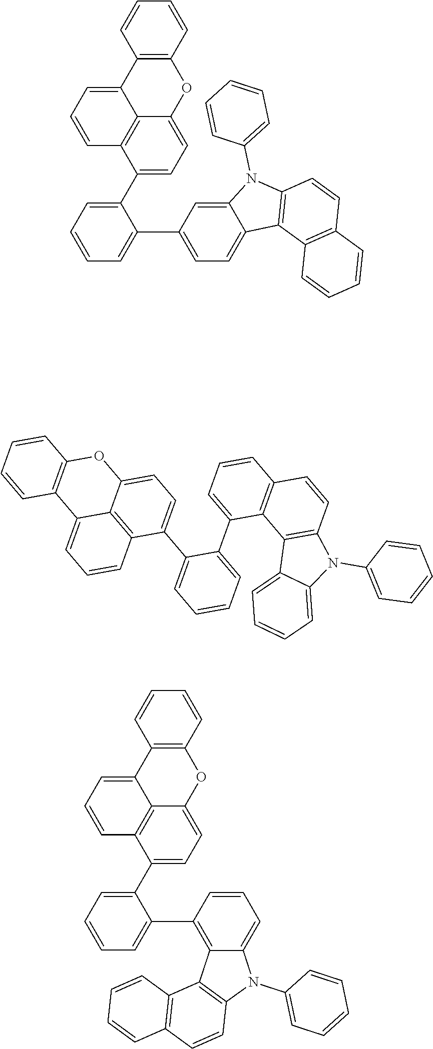 [SMILES: c1ccc(-n2c3cc(-c4ccccc4-c4ccc5c6c(cccc46)-c4ccccc4O5)ccc3c3c4ccccc4ccc32)cc1 ; c1ccc(-n2c3ccccc3c3c4c(-c5ccccc5-c5ccc6c7c(cccc57)-c5ccccc5O6)cccc4ccc32)cc1 ; c1ccc(-n2c3cccc(-c4ccccc4-c4ccc5c6c(cccc46)-c4ccccc4O5)c3c3c4ccccc4ccc32)cc1]

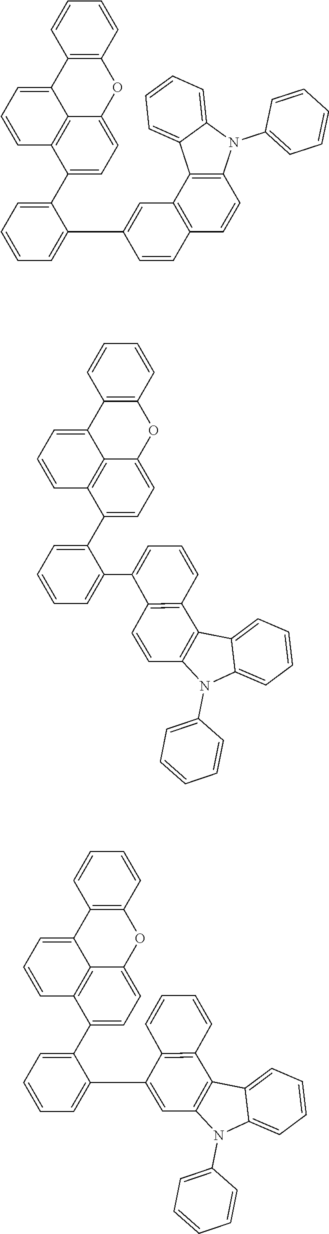 [SMILES: c1ccc(-n2c3ccccc3c3c4cc(-c5ccccc5-c5ccc6c7c(cccc57)-c5ccccc5O6)ccc4ccc32)cc1 ; c1ccc(-n2c3ccccc3c3c4cccc(-c5ccccc5-c5ccc6c7c(cccc57)-c5ccccc5O6)c4ccc32)cc1 ; c1ccc(-n2c3ccccc3c3c4ccccc4c(-c4ccccc4-c4ccc5c6c(cccc46)-c4ccccc4O5)cc32)cc1]

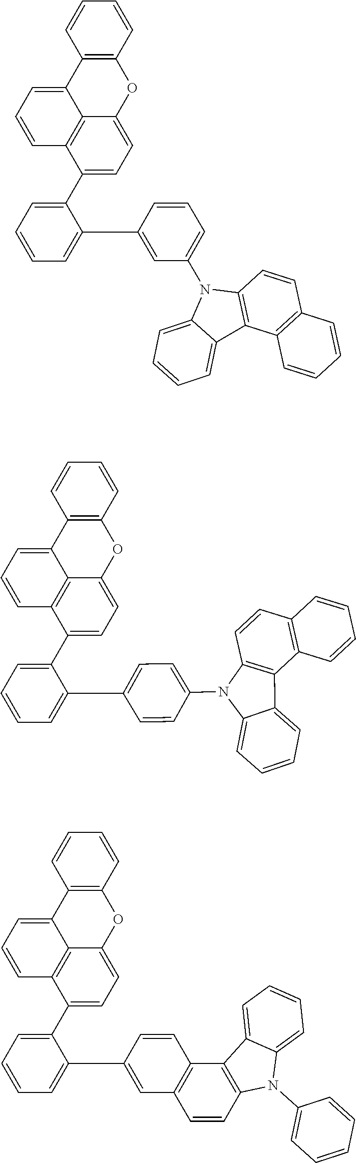 [SMILES: c1cc(-c2ccccc2-c2ccc3c4c(cccc24)-c2ccccc2O3)cc(-n2c3ccccc3c3c4ccccc4ccc32)c1 ; c1ccc2c(c1)Oc1ccc(-c3ccccc3-c3ccc(-n4c5ccccc5c5c6ccccc6ccc54)cc3)c3cccc-2c13 ; c1ccc(-n2c3ccccc3c3c4ccc(-c5ccccc5-c5ccc6c7c(cccc57)-c5ccccc5O6)cc4ccc32)cc1]

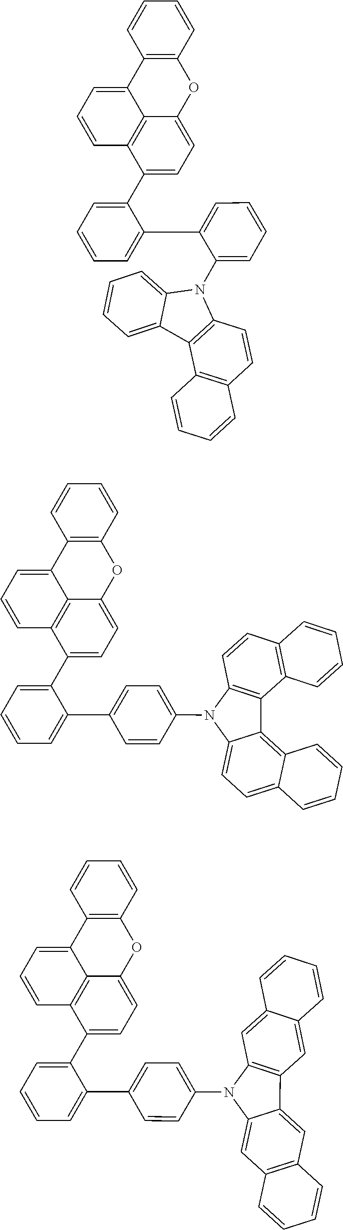 [SMILES: c1ccc2c(c1)Oc1ccc(-c3ccccc3-c3ccccc3-n3c4ccccc4c4c5ccccc5ccc43)c3cccc-2c13 ; c1ccc2c(c1)Oc1ccc(-c3ccccc3-c3ccc(-n4c5ccc6ccccc6c5c5c6ccccc6ccc54)cc3)c3cccc-2c13 ; c1ccc2c(c1)Oc1ccc(-c3ccccc3-c3ccc(-n4c5cc6ccccc6cc5c5cc6ccccc6cc54)cc3)c3cccc-2c13]

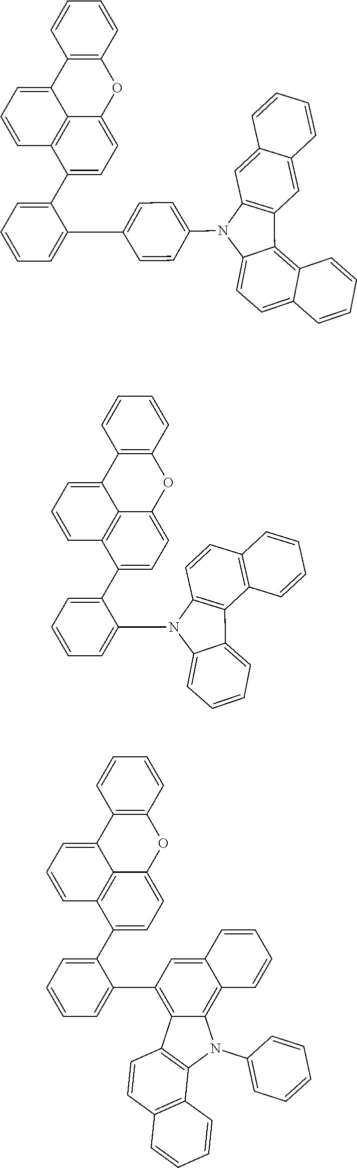 [SMILES: c1ccc2c(c1)Oc1ccc(-c3ccccc3-c3ccc(-n4c5cc6ccccc6cc5c5c6ccccc6ccc54)cc3)c3cccc-2c13 ; c1ccc2c(c1)Oc1ccc(-c3ccccc3-n3c4ccccc4c4c5ccccc5ccc43)c3cccc-2c13 ; c1ccc(-n2c3c4ccccc4ccc3c3c(-c4ccccc4-c4ccc5c6c(cccc46)-c4ccccc4O5)cc4ccccc4c32)cc1]

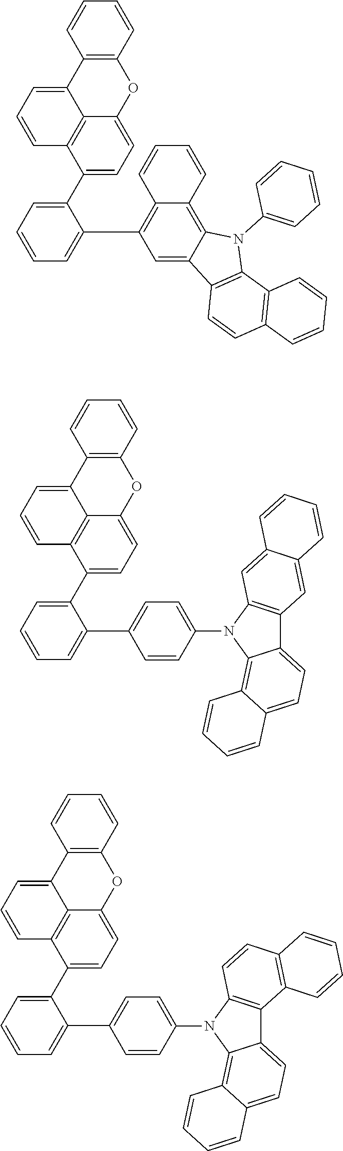 [SMILES: c1ccc(-n2c3c4ccccc4ccc3c3cc(-c4ccccc4-c4ccc5c6c(cccc46)-c4ccccc4O5)c4ccccc4c32)cc1 ; c1ccc2c(c1)Oc1ccc(-c3ccccc3-c3ccc(-n4c5cc6ccccc6cc5c5ccc6ccccc6c54)cc3)c3cccc-2c13 ; c1ccc2c(c1)Oc1ccc(-c3ccccc3-c3ccc(-n4c5ccc6ccccc6c5c5ccc6ccccc6c54)cc3)c3cccc-2c13]

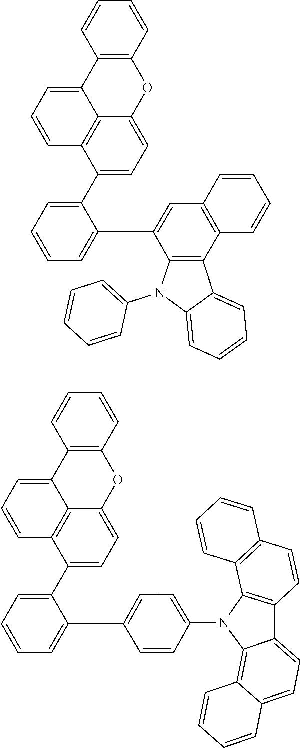 [SMILES: c1ccc(-n2c3ccccc3c3c4ccccc4cc(-c4ccccc4-c4ccc5c6c(cccc46)-c4ccccc4O5)c32)cc1 ; c1ccc2c(c1)Oc1ccc(-c3ccccc3-c3ccc(-n4c5c6ccccc6ccc5c5ccc6ccccc6c54)cc3)c3cccc-2c13]

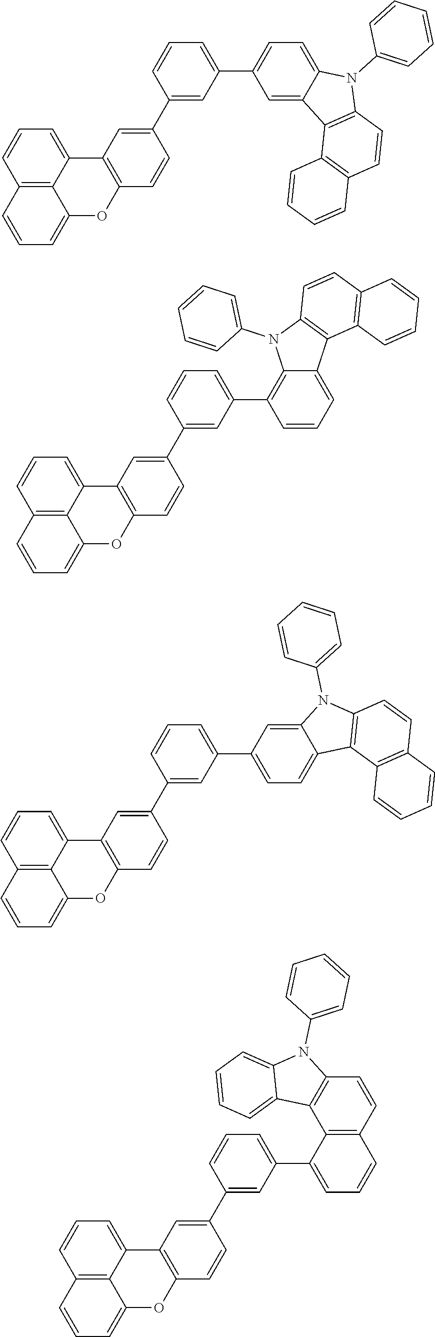 [SMILES: c1ccc(-n2c3ccc(-c4cccc(-c5ccc6c(c5)-c5cccc7cccc(c57)O6)c4)cc3c3c4ccccc4ccc32)cc1 ; c1ccc(-n2c3ccc4ccccc4c3c3cccc(-c4cccc(-c5ccc6c(c5)-c5cccc7cccc(c57)O6)c4)c32)cc1 ; c1ccc(-n2c3cc(-c4cccc(-c5ccc6c(c5)-c5cccc7cccc(c57)O6)c4)ccc3c3c4ccccc4ccc32)cc1 ; c1ccc(-n2c3ccccc3c3c4c(-c5cccc(-c6ccc7c(c6)-c6cccc8cccc(c68)O7)c5)cccc4ccc32)cc1]

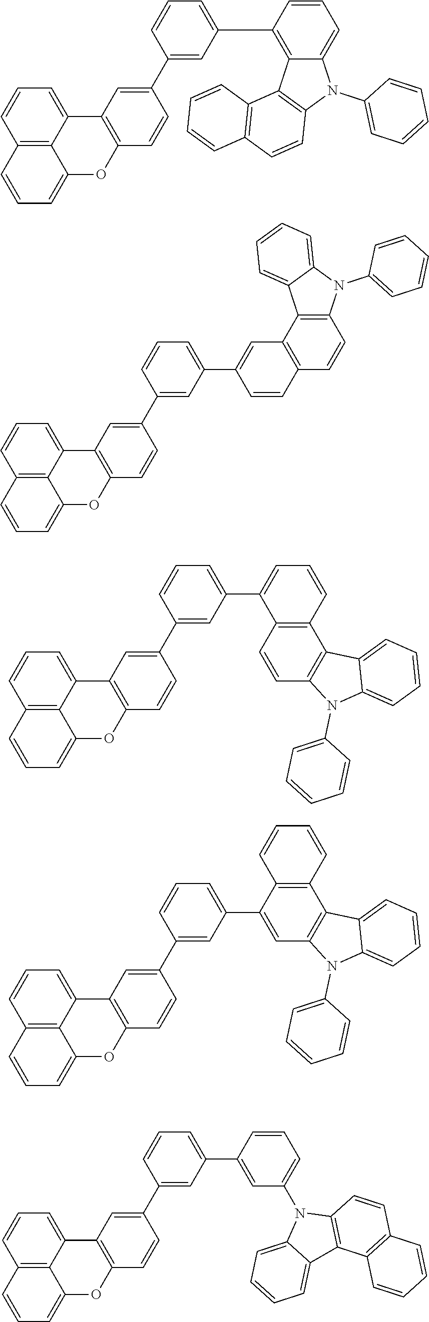

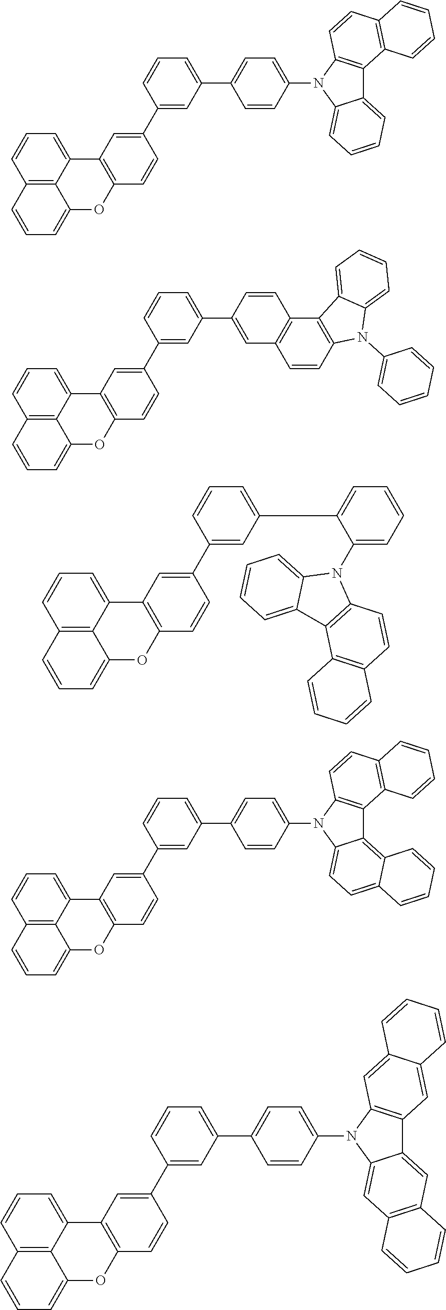

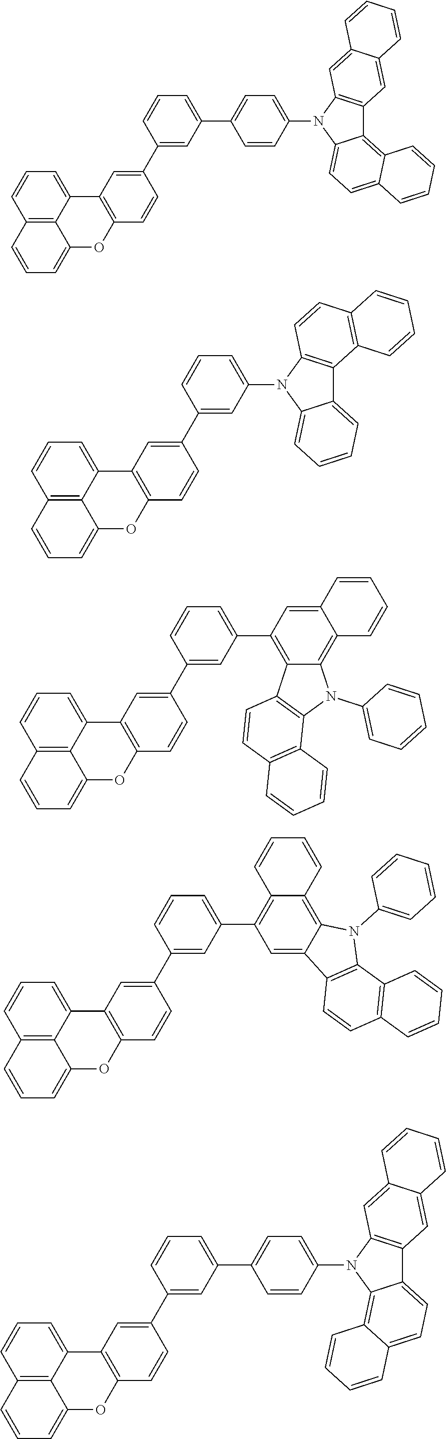

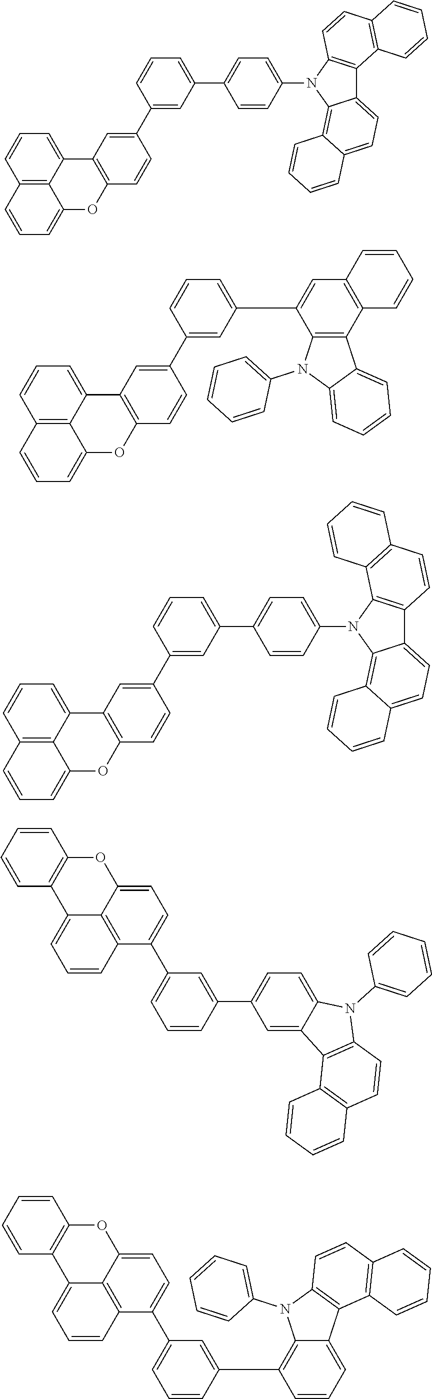

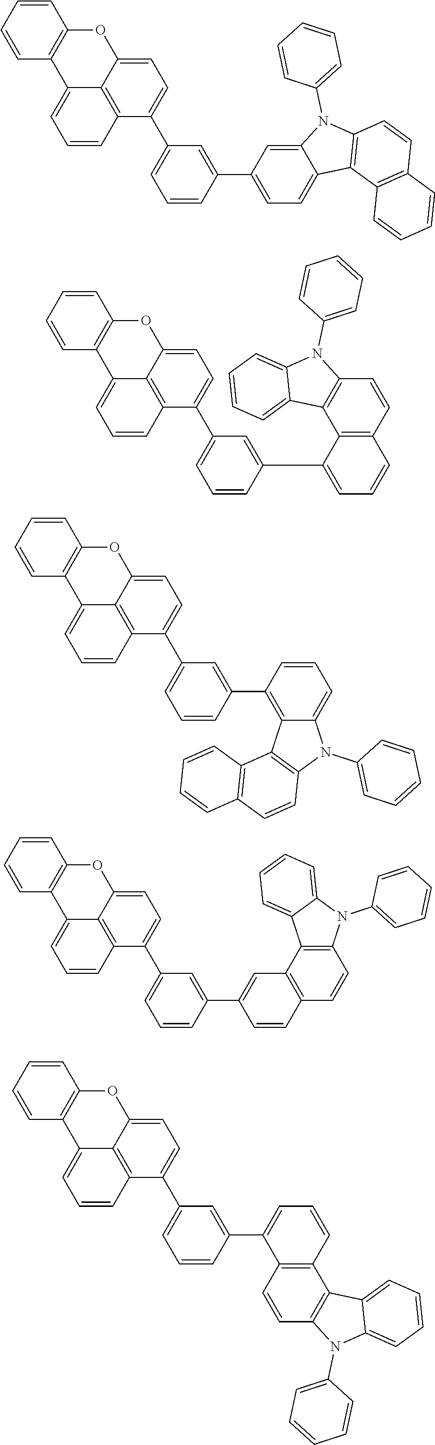

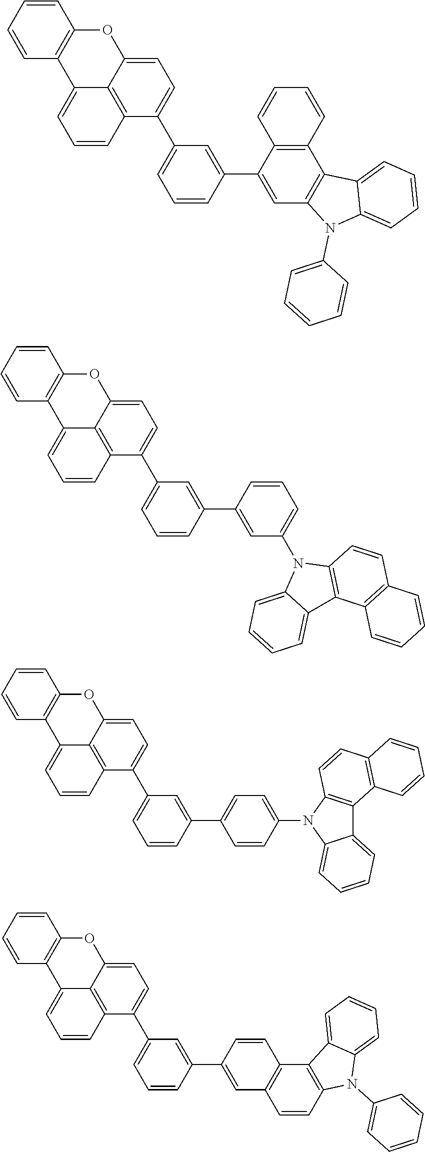 [SMILES: c1ccc(-n2c3ccccc3c3c4ccccc4c(-c4cccc(-c5ccc6c7c(cccc57)-c5ccccc5O6)c4)cc32)cc1 ; c1cc(-c2cccc(-n3c4ccccc4c4c5ccccc5ccc43)c2)cc(-c2ccc3c4c(cccc24)-c2ccccc2O3)c1 ; c1cc(-c2ccc(-n3c4ccccc4c4c5ccccc5ccc43)cc2)cc(-c2ccc3c4c(cccc24)-c2ccccc2O3)c1 ; c1ccc(-n2c3ccccc3c3c4ccc(-c5cccc(-c6ccc7c8c(cccc68)-c6ccccc6O7)c5)cc4ccc32)cc1]

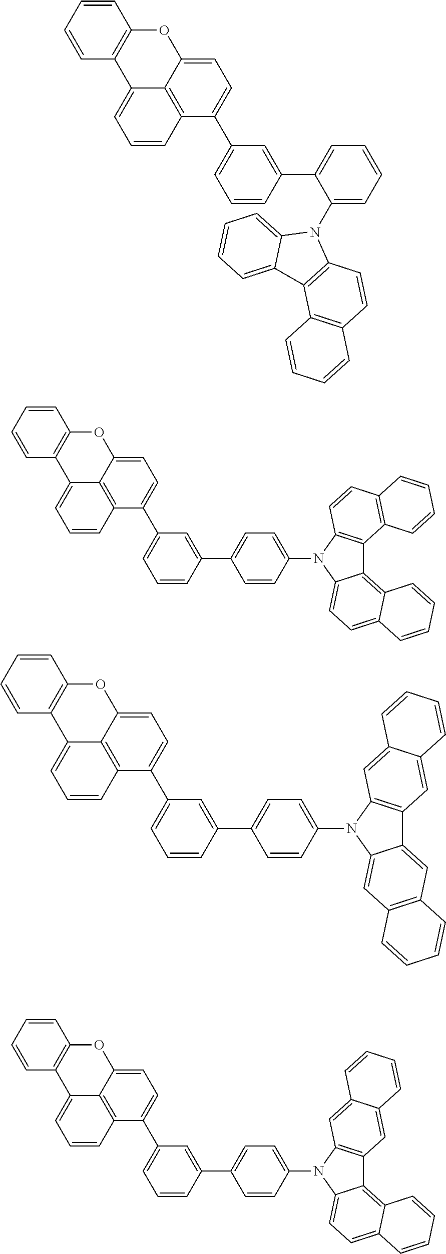 [SMILES: c1cc(-c2ccccc2-n2c3ccccc3c3c4ccccc4ccc32)cc(-c2ccc3c4c(cccc24)-c2ccccc2O3)c1 ; c1cc(-c2ccc(-n3c4ccc5ccccc5c4c4c5ccccc5ccc43)cc2)cc(-c2ccc3c4c(cccc24)-c2ccccc2O3)c1 ; c1cc(-c2ccc(-n3c4cc5ccccc5cc4c4cc5ccccc5cc43)cc2)cc(-c2ccc3c4c(cccc24)-c2ccccc2O3)c1 ; c1cc(-c2ccc(-n3c4cc5ccccc5cc4c4c5ccccc5ccc43)cc2)cc(-c2ccc3c4c(cccc24)-c2ccccc2O3)c1]

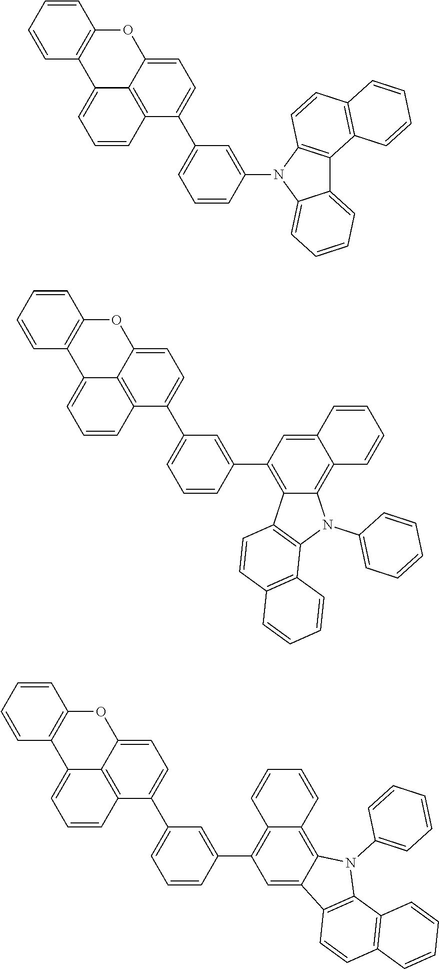 [SMILES: c1cc(-c2ccc3c4c(cccc24)-c2ccccc2O3)cc(-n2c3ccccc3c3c4ccccc4ccc32)c1 ; c1ccc(-n2c3c4ccccc4ccc3c3c(-c4cccc(-c5ccc6c7c(cccc57)-c5ccccc5O6)c4)cc4ccccc4c32)cc1 ; c1ccc(-n2c3c4ccccc4ccc3c3cc(-c4cccc(-c5ccc6c7c(cccc57)-c5ccccc5O6)c4)c4ccccc4c32)cc1]

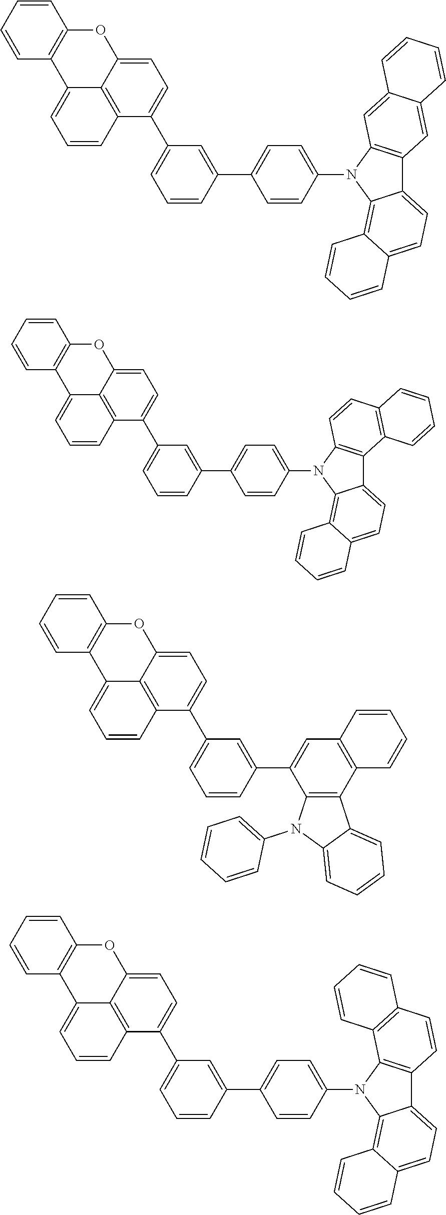 [SMILES: c1cc(-c2ccc(-n3c4cc5ccccc5cc4c4ccc5ccccc5c43)cc2)cc(-c2ccc3c4c(cccc24)-c2ccccc2O3)c1 ; c1cc(-c2ccc(-n3c4ccc5ccccc5c4c4ccc5ccccc5c43)cc2)cc(-c2ccc3c4c(cccc24)-c2ccccc2O3)c1 ; c1ccc(-n2c3ccccc3c3c4ccccc4cc(-c4cccc(-c5ccc6c7c(cccc57)-c5ccccc5O6)c4)c32)cc1 ; c1cc(-c2ccc(-n3c4c5ccccc5ccc4c4ccc5ccccc5c43)cc2)cc(-c2ccc3c4c(cccc24)-c2ccccc2O3)c1]

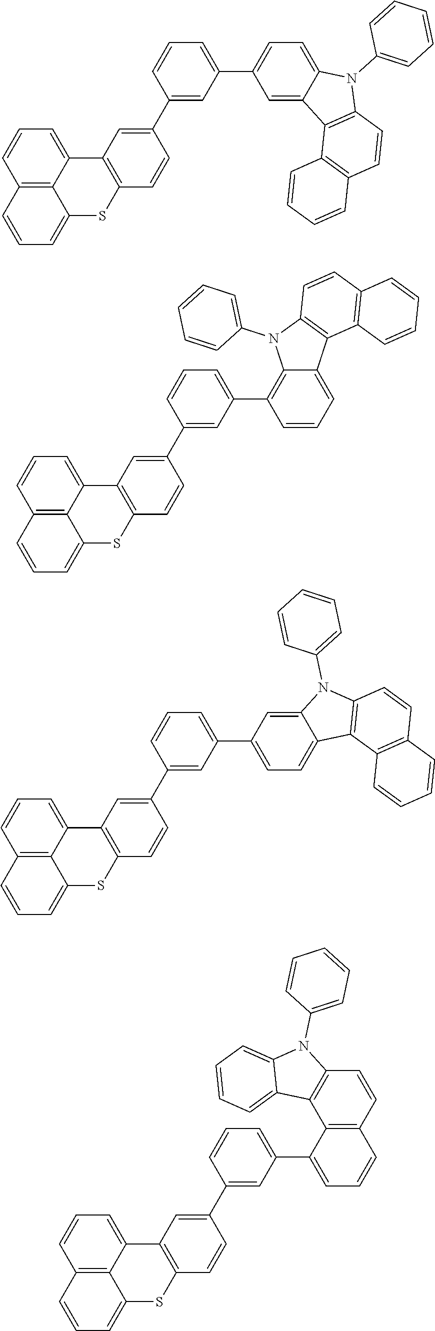 [SMILES: c1ccc(-n2c3ccc(-c4cccc(-c5ccc6c(c5)-c5cccc7cccc(c57)S6)c4)cc3c3c4ccccc4ccc32)cc1 ; c1ccc(-n2c3ccc4ccccc4c3c3cccc(-c4cccc(-c5ccc6c(c5)-c5cccc7cccc(c57)S6)c4)c32)cc1 ; c1ccc(-n2c3cc(-c4cccc(-c5ccc6c(c5)-c5cccc7cccc(c57)S6)c4)ccc3c3c4ccccc4ccc32)cc1 ; c1ccc(-n2c3ccccc3c3c4c(-c5cccc(-c6ccc7c(c6)-c6cccc8cccc(c68)S7)c5)cccc4ccc32)cc1]

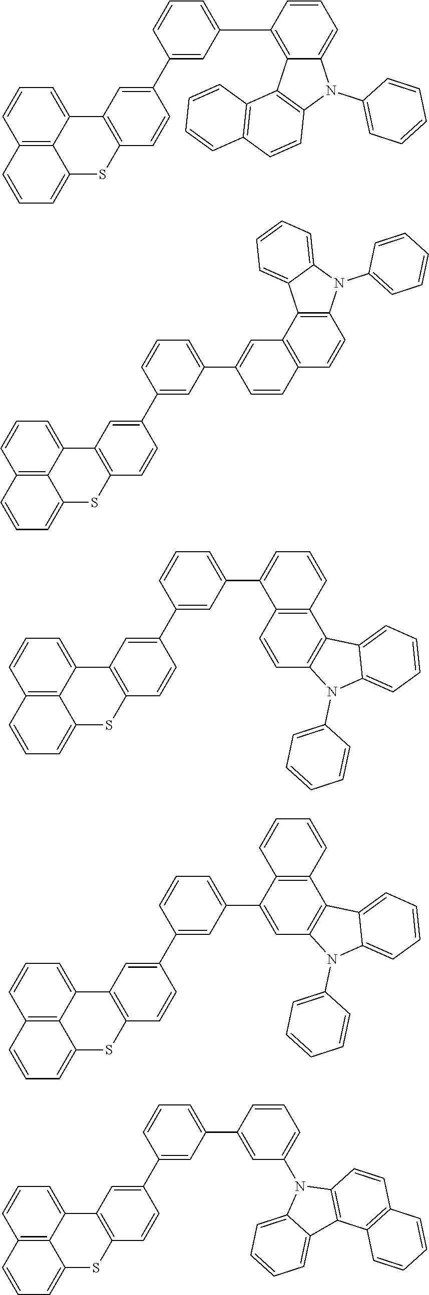

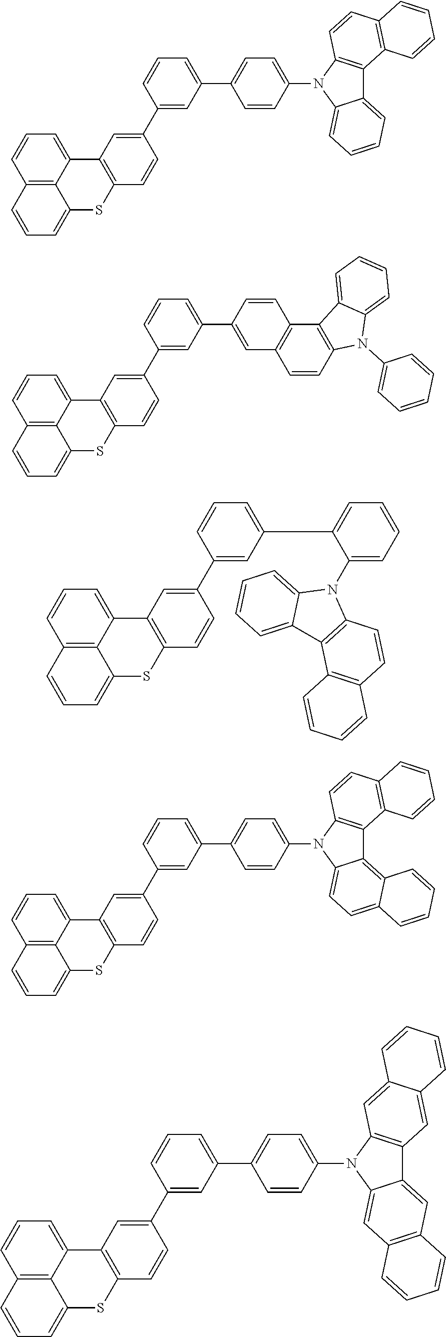

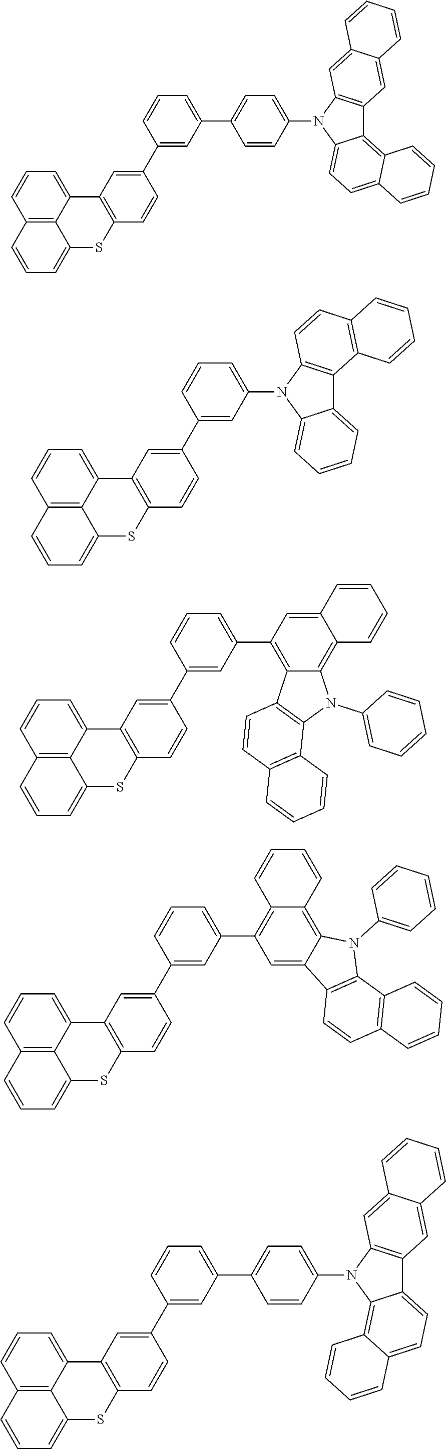

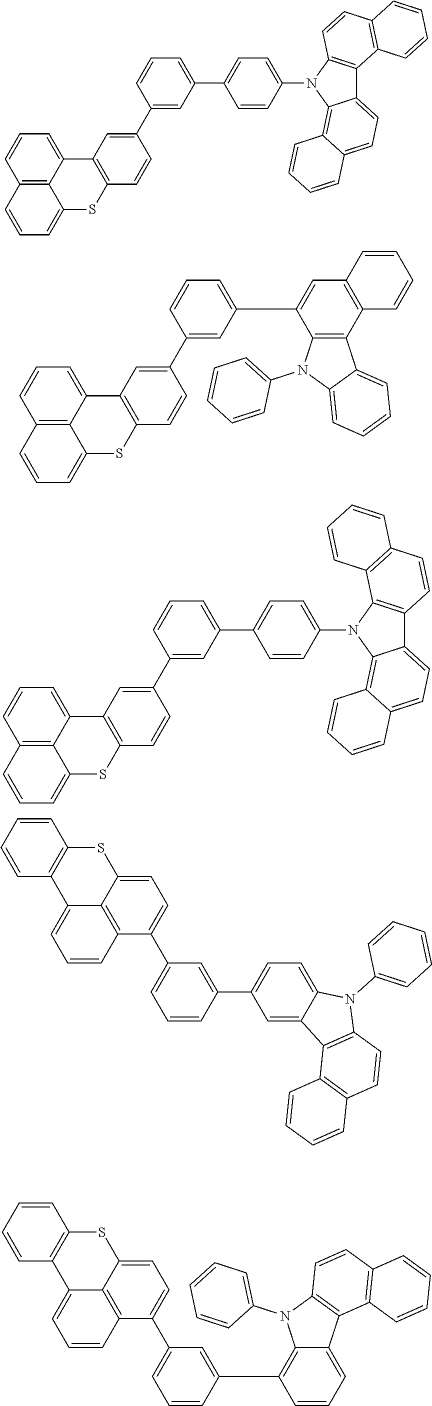

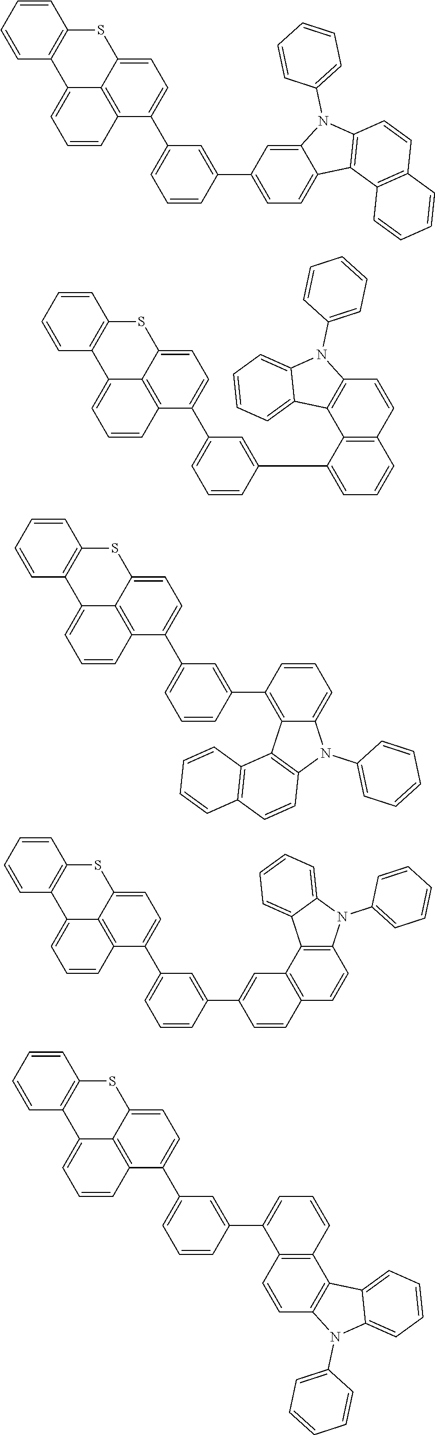

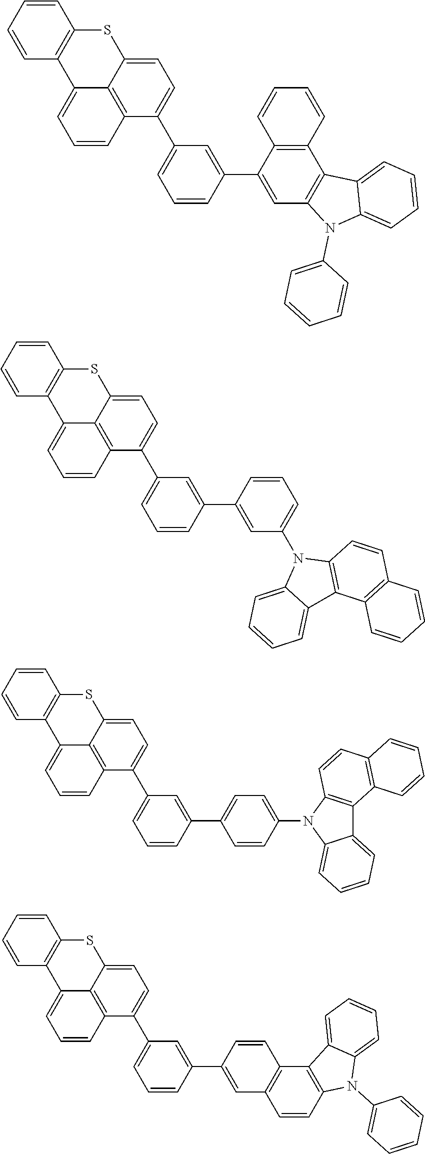 [SMILES: c1ccc(-n2c3ccccc3c3c4ccccc4c(-c4cccc(-c5ccc6c7c(cccc57)-c5ccccc5S6)c4)cc32)cc1 ; c1cc(-c2cccc(-n3c4ccccc4c4c5ccccc5ccc43)c2)cc(-c2ccc3c4c(cccc24)-c2ccccc2S3)c1 ; c1cc(-c2ccc(-n3c4ccccc4c4c5ccccc5ccc43)cc2)cc(-c2ccc3c4c(cccc24)-c2ccccc2S3)c1 ; c1ccc(-n2c3ccccc3c3c4ccc(-c5cccc(-c6ccc7c8c(cccc68)-c6ccccc6S7)c5)cc4ccc32)cc1]

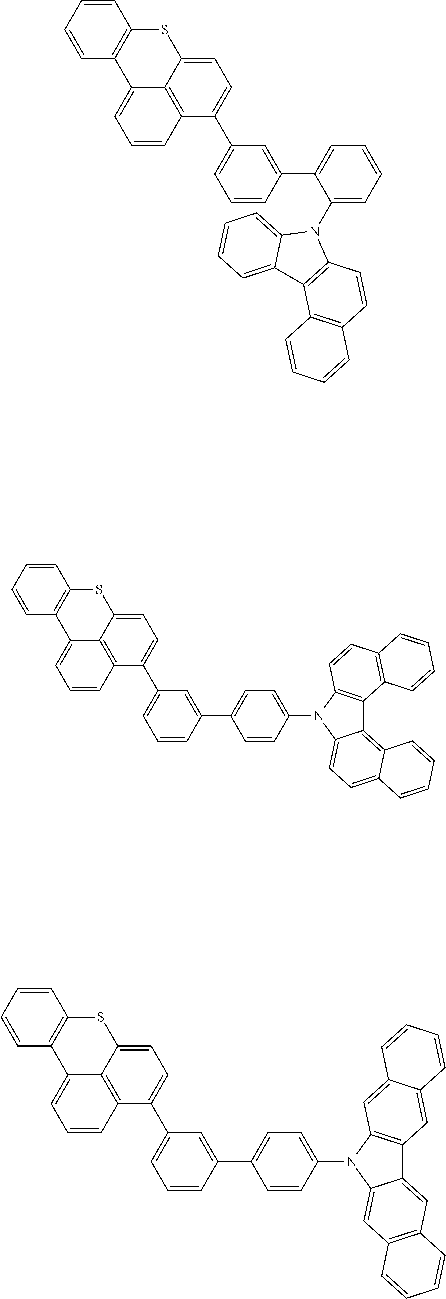 [SMILES: c1cc(-c2ccccc2-n2c3ccccc3c3c4ccccc4ccc32)cc(-c2ccc3c4c(cccc24)-c2ccccc2S3)c1 ; c1cc(-c2ccc(-n3c4ccc5ccccc5c4c4c5ccccc5ccc43)cc2)cc(-c2ccc3c4c(cccc24)-c2ccccc2S3)c1 ; c1cc(-c2ccc(-n3c4cc5ccccc5cc4c4cc5ccccc5cc43)cc2)cc(-c2ccc3c4c(cccc24)-c2ccccc2S3)c1]

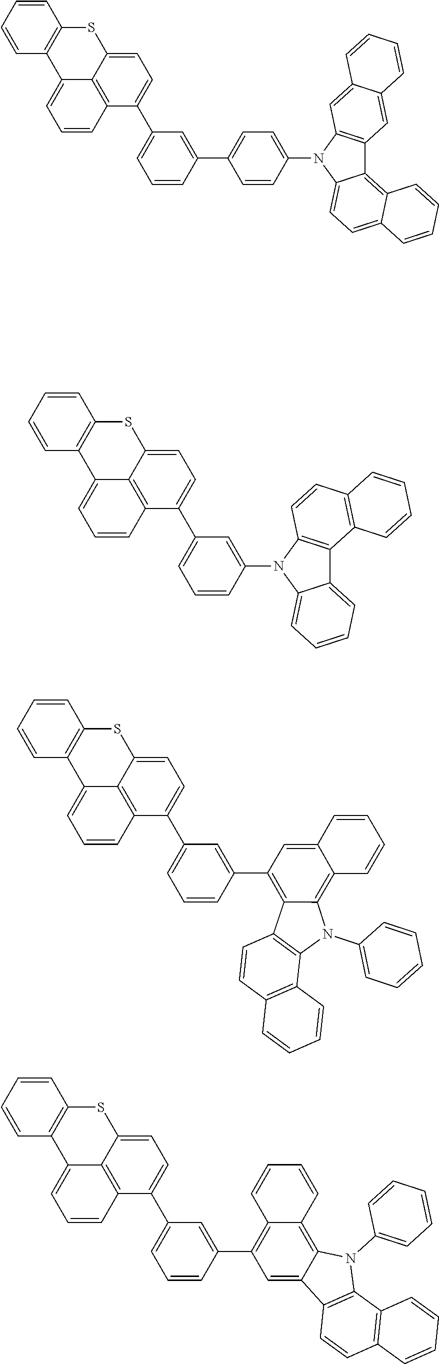 [SMILES: c1cc(-c2ccc(-n3c4cc5ccccc5cc4c4c5ccccc5ccc43)cc2)cc(-c2ccc3c4c(cccc24)-c2ccccc2S3)c1 ; c1cc(-c2ccc3c4c(cccc24)-c2ccccc2S3)cc(-n2c3ccccc3c3c4ccccc4ccc32)c1 ; c1ccc(-n2c3c4ccccc4ccc3c3c(-c4cccc(-c5ccc6c7c(cccc57)-c5ccccc5S6)c4)cc4ccccc4c32)cc1 ; c1ccc(-n2c3c4ccccc4ccc3c3cc(-c4cccc(-c5ccc6c7c(cccc57)-c5ccccc5S6)c4)c4ccccc4c32)cc1]

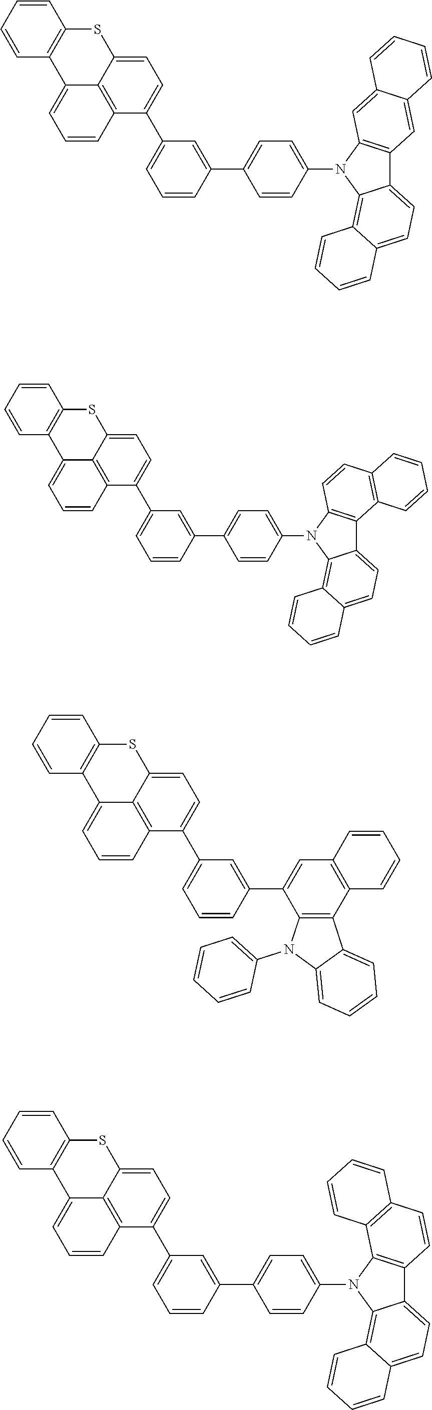 [SMILES: c1cc(-c2ccc(-n3c4cc5ccccc5cc4c4ccc5ccccc5c43)cc2)cc(-c2ccc3c4c(cccc24)-c2ccccc2S3)c1 ; c1cc(-c2ccc(-n3c4ccc5ccccc5c4c4ccc5ccccc5c43)cc2)cc(-c2ccc3c4c(cccc24)-c2ccccc2S3)c1 ; c1ccc(-n2c3ccccc3c3c4ccccc4cc(-c4cccc(-c5ccc6c7c(cccc57)-c5ccccc5S6)c4)c32)cc1 ; c1cc(-c2ccc(-n3c4c5ccccc5ccc4c4ccc5ccccc5c43)cc2)cc(-c2ccc3c4c(cccc24)-c2ccccc2S3)c1]

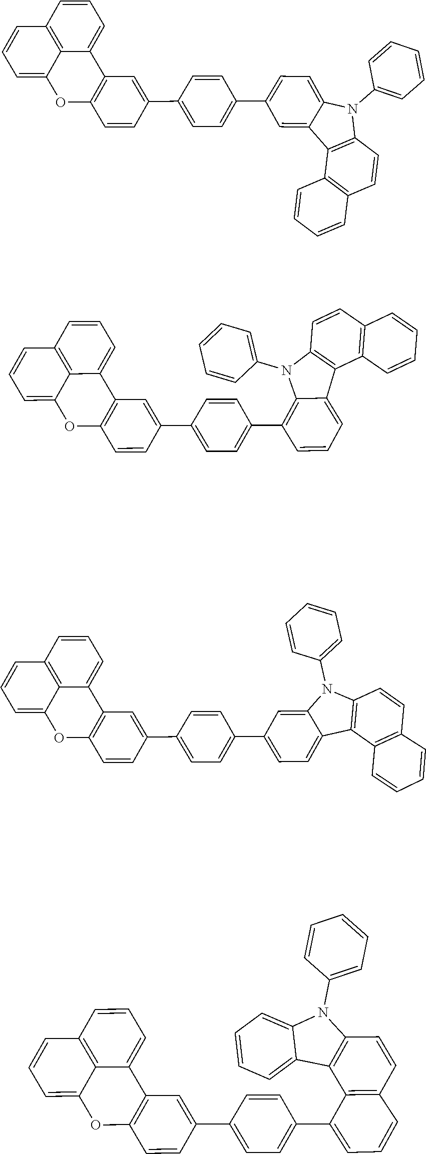 [SMILES: c1ccc(-n2c3ccc(-c4ccc(-c5ccc6c(c5)-c5cccc7cccc(c57)O6)cc4)cc3c3c4ccccc4ccc32)cc1 ; c1ccc(-n2c3ccc4ccccc4c3c3cccc(-c4ccc(-c5ccc6c(c5)-c5cccc7cccc(c57)O6)cc4)c32)cc1 ; c1ccc(-n2c3cc(-c4ccc(-c5ccc6c(c5)-c5cccc7cccc(c57)O6)cc4)ccc3c3c4ccccc4ccc32)cc1 ; c1ccc(-n2c3ccccc3c3c4c(-c5ccc(-c6ccc7c(c6)-c6cccc8cccc(c68)O7)cc5)cccc4ccc32)cc1]

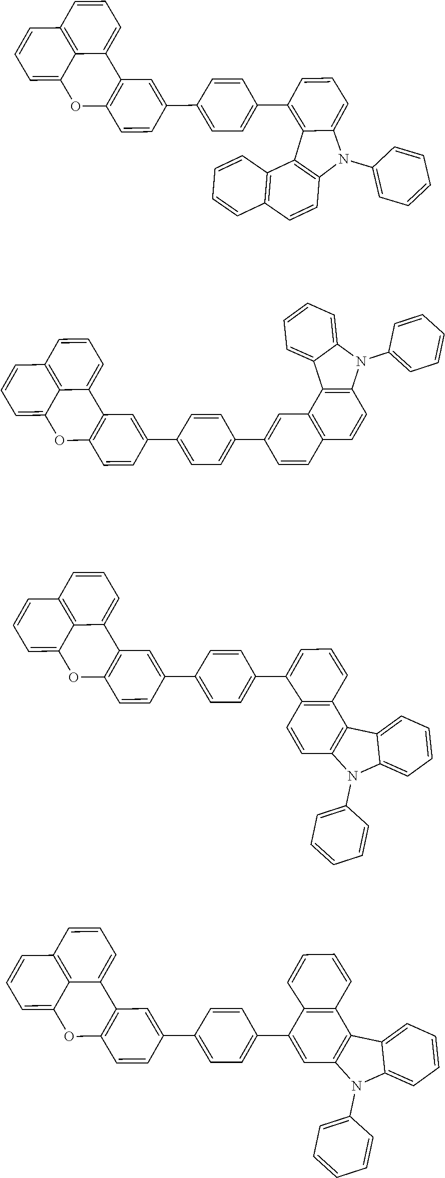 [SMILES: c1ccc(-n2c3cccc(-c4ccc(-c5ccc6c(c5)-c5cccc7cccc(c57)O6)cc4)c3c3c4ccccc4ccc32)cc1 ; c1ccc(-n2c3ccccc3c3c4cc(-c5ccc(-c6ccc7c(c6)-c6cccc8cccc(c68)O7)cc5)ccc4ccc32)cc1 ; c1ccc(-n2c3ccccc3c3c4cccc(-c5ccc(-c6ccc7c(c6)-c6cccc8cccc(c68)O7)cc5)c4ccc32)cc1 ; c1ccc(-n2c3ccccc3c3c4ccccc4c(-c4ccc(-c5ccc6c(c5)-c5cccc7cccc(c57)O6)cc4)cc32)cc1]

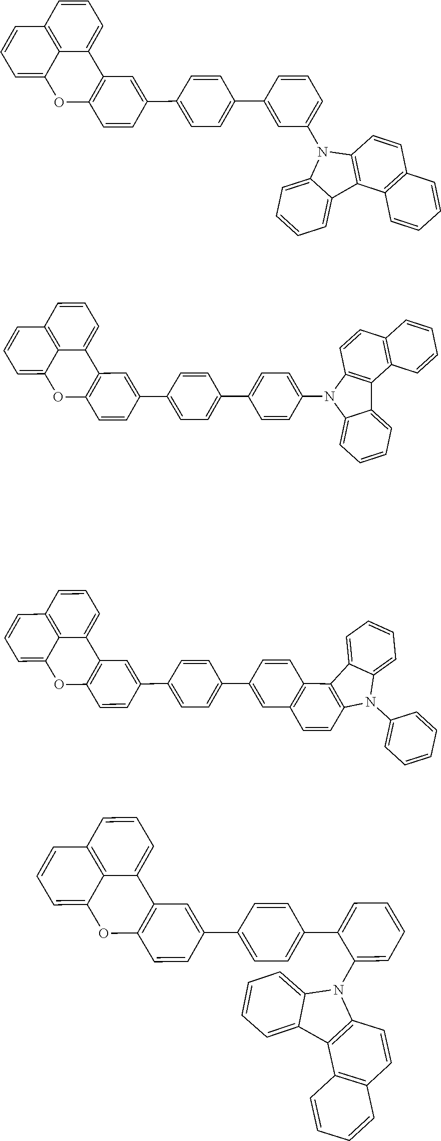 [SMILES: c1cc(-c2ccc(-c3ccc4c(c3)-c3cccc5cccc(c35)O4)cc2)cc(-n2c3ccccc3c3c4ccccc4ccc32)c1 ; c1cc2c3c(cccc3c1)-c1cc(-c3ccc(-c4ccc(-n5c6ccccc6c6c7ccccc7ccc65)cc4)cc3)ccc1O2 ; c1ccc(-n2c3ccccc3c3c4ccc(-c5ccc(-c6ccc7c(c6)-c6cccc8cccc(c68)O7)cc5)cc4ccc32)cc1 ; c1ccc(-n2c3ccccc3c3c4ccccc4ccc32)c(-c2ccc(-c3ccc4c(c3)-c3cccc5cccc(c35)O4)cc2)c1]

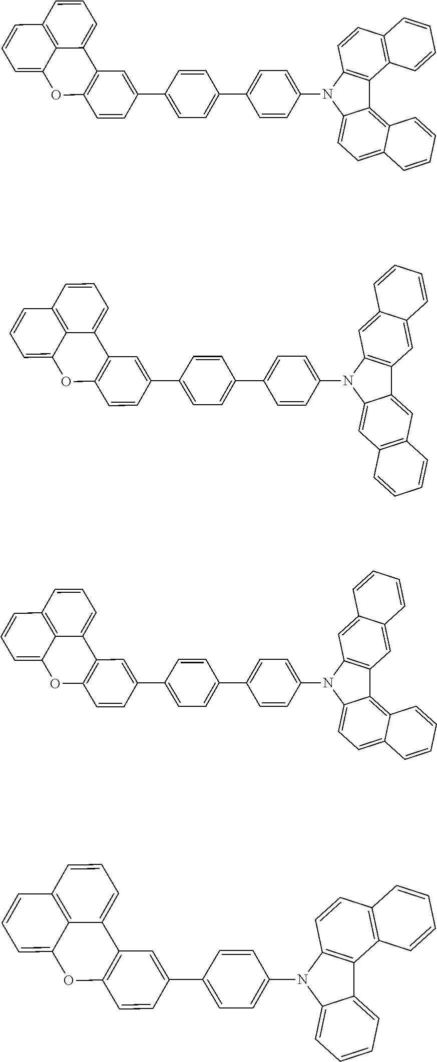 [SMILES: c1cc2c3c(cccc3c1)-c1cc(-c3ccc(-c4ccc(-n5c6ccc7ccccc7c6c6c7ccccc7ccc65)cc4)cc3)ccc1O2 ; c1ccc2cc3c(cc2c1)c1cc2ccccc2cc1n3-c1ccc(-c2ccc(-c3ccc4c(c3)-c3cccc5cccc(c35)O4)cc2)cc1 ; c1ccc2cc3c(cc2c1)c1c2ccccc2ccc1n3-c1ccc(-c2ccc(-c3ccc4c(c3)-c3cccc5cccc(c35)O4)cc2)cc1 ; c1cc2c3c(cccc3c1)-c1cc(-c3ccc(-n4c5ccccc5c5c6ccccc6ccc54)cc3)ccc1O2]

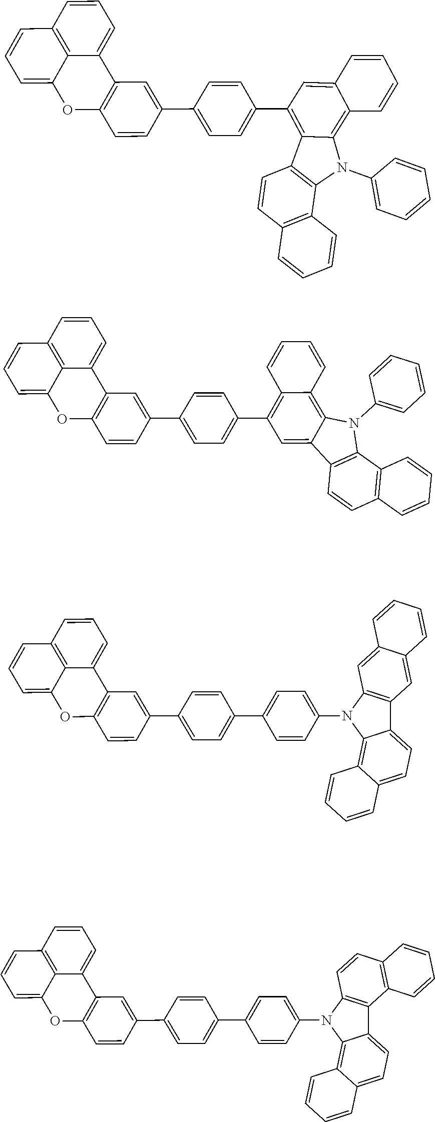 [SMILES: c1ccc(-n2c3c4ccccc4ccc3c3c(-c4ccc(-c5ccc6c(c5)-c5cccc7cccc(c57)O6)cc4)cc4ccccc4c32)cc1 ; c1ccc(-n2c3c4ccccc4ccc3c3cc(-c4ccc(-c5ccc6c(c5)-c5cccc7cccc(c57)O6)cc4)c4ccccc4c32)cc1 ; c1ccc2cc3c(cc2c1)c1ccc2ccccc2c1n3-c1ccc(-c2ccc(-c3ccc4c(c3)-c3cccc5cccc(c35)O4)cc2)cc1 ; c1cc2c3c(cccc3c1)-c1cc(-c3ccc(-c4ccc(-n5c6ccc7ccccc7c6c6ccc7ccccc7c65)cc4)cc3)ccc1O2]

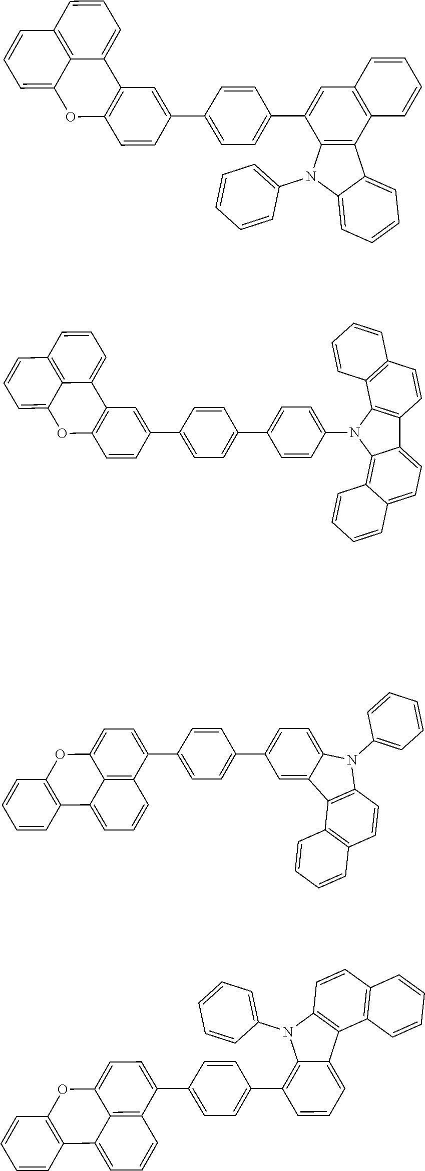 [SMILES: c1ccc(-n2c3ccccc3c3c4ccccc4cc(-c4ccc(-c5ccc6c(c5)-c5cccc7cccc(c57)O6)cc4)c32)cc1 ; c1cc2c3c(cccc3c1)-c1cc(-c3ccc(-c4ccc(-n5c6c7ccccc7ccc6c6ccc7ccccc7c65)cc4)cc3)ccc1O2 ; c1ccc(-n2c3ccc(-c4ccc(-c5ccc6c7c(cccc57)-c5ccccc5O6)cc4)cc3c3c4ccccc4ccc32)cc1 ; c1ccc(-n2c3ccc4ccccc4c3c3cccc(-c4ccc(-c5ccc6c7c(cccc57)-c5ccccc5O6)cc4)c32)cc1]

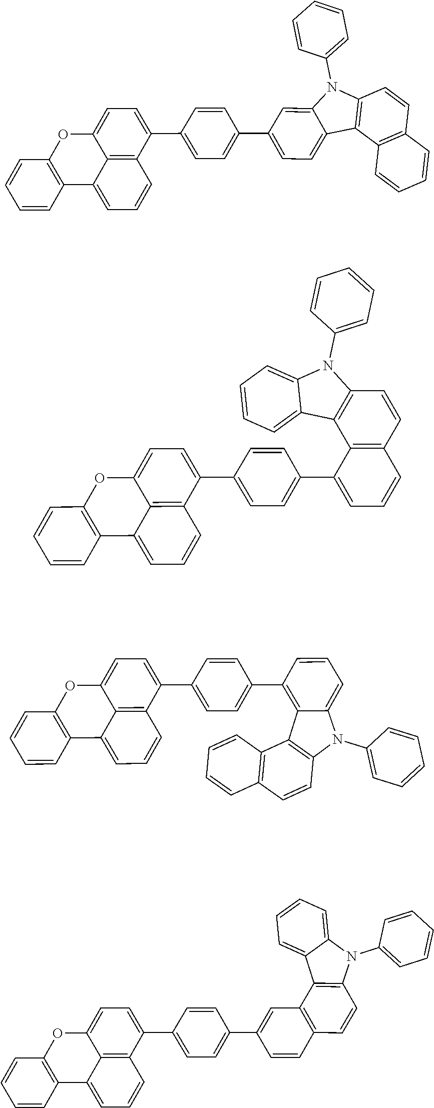 [SMILES: c1ccc(-n2c3cc(-c4ccc(-c5ccc6c7c(cccc57)-c5ccccc5O6)cc4)ccc3c3c4ccccc4ccc32)cc1 ; c1ccc(-n2c3ccccc3c3c4c(-c5ccc(-c6ccc7c8c(cccc68)-c6ccccc6O7)cc5)cccc4ccc32)cc1 ; c1ccc(-n2c3cccc(-c4ccc(-c5ccc6c7c(cccc57)-c5ccccc5O6)cc4)c3c3c4ccccc4ccc32)cc1 ; c1ccc(-n2c3ccccc3c3c4cc(-c5ccc(-c6ccc7c8c(cccc68)-c6ccccc6O7)cc5)ccc4ccc32)cc1]

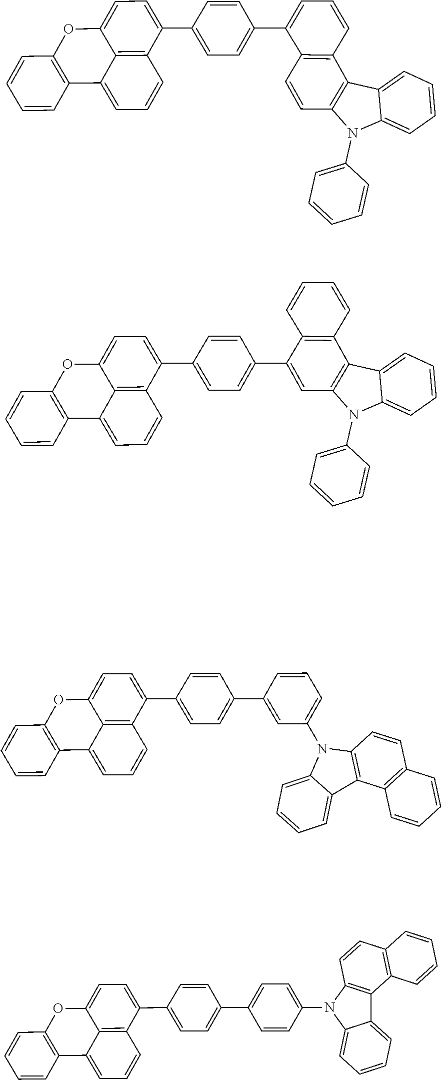 [SMILES: c1ccc(-n2c3ccccc3c3c4cccc(-c5ccc(-c6ccc7c8c(cccc68)-c6ccccc6O7)cc5)c4ccc32)cc1 ; c1ccc(-n2c3ccccc3c3c4ccccc4c(-c4ccc(-c5ccc6c7c(cccc57)-c5ccccc5O6)cc4)cc32)cc1 ; c1cc(-c2ccc(-c3ccc4c5c(cccc35)-c3ccccc3O4)cc2)cc(-n2c3ccccc3c3c4ccccc4ccc32)c1 ; c1ccc2c(c1)Oc1ccc(-c3ccc(-c4ccc(-n5c6ccccc6c6c7ccccc7ccc65)cc4)cc3)c3cccc-2c13]

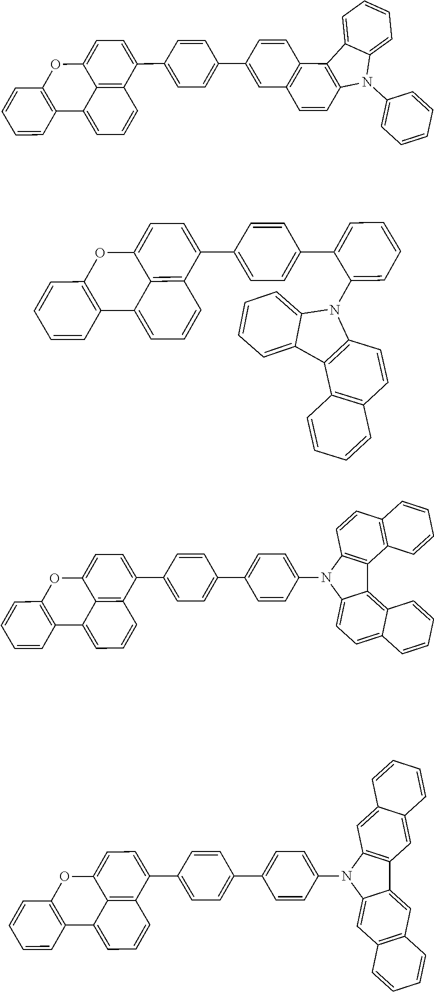 [SMILES: c1ccc(-n2c3ccccc3c3c4ccc(-c5ccc(-c6ccc7c8c(cccc68)-c6ccccc6O7)cc5)cc4ccc32)cc1 ; c1ccc2c(c1)Oc1ccc(-c3ccc(-c4ccccc4-n4c5ccccc5c5c6ccccc6ccc54)cc3)c3cccc-2c13 ; c1ccc2c(c1)Oc1ccc(-c3ccc(-c4ccc(-n5c6ccc7ccccc7c6c6c7ccccc7ccc65)cc4)cc3)c3cccc-2c13 ; c1ccc2c(c1)Oc1ccc(-c3ccc(-c4ccc(-n5c6cc7ccccc7cc6c6cc7ccccc7cc65)cc4)cc3)c3cccc-2c13]

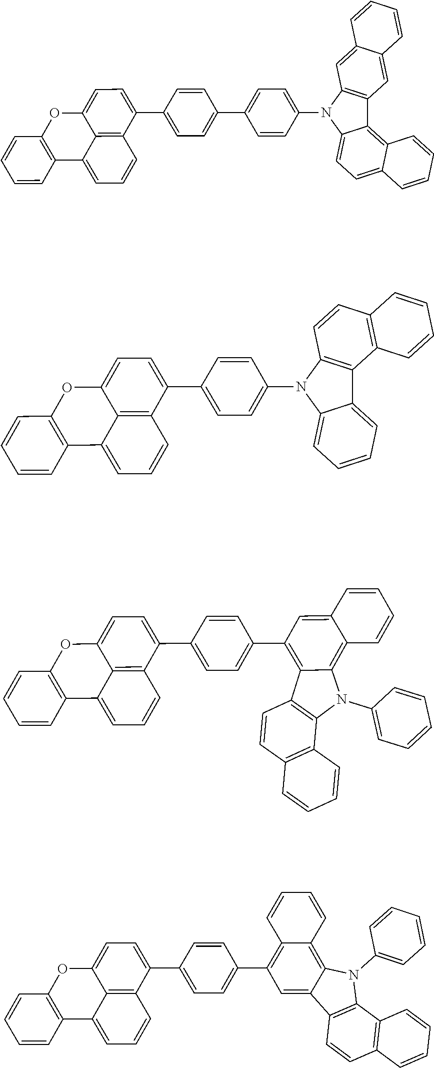 [SMILES: c1ccc2c(c1)Oc1ccc(-c3ccc(-c4ccc(-n5c6cc7ccccc7cc6c6c7ccccc7ccc65)cc4)cc3)c3cccc-2c13 ; c1ccc2c(c1)Oc1ccc(-c3ccc(-n4c5ccccc5c5c6ccccc6ccc54)cc3)c3cccc-2c13 ; c1ccc(-n2c3c4ccccc4ccc3c3c(-c4ccc(-c5ccc6c7c(cccc57)-c5ccccc5O6)cc4)cc4ccccc4c32)cc1 ; c1ccc(-n2c3c4ccccc4ccc3c3cc(-c4ccc(-c5ccc6c7c(cccc57)-c5ccccc5O6)cc4)c4ccccc4c32)cc1]

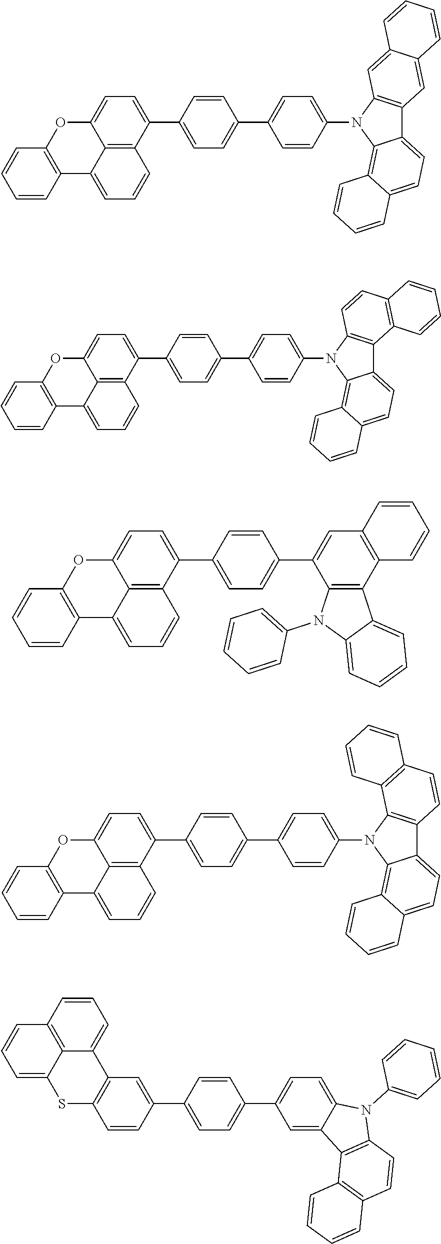

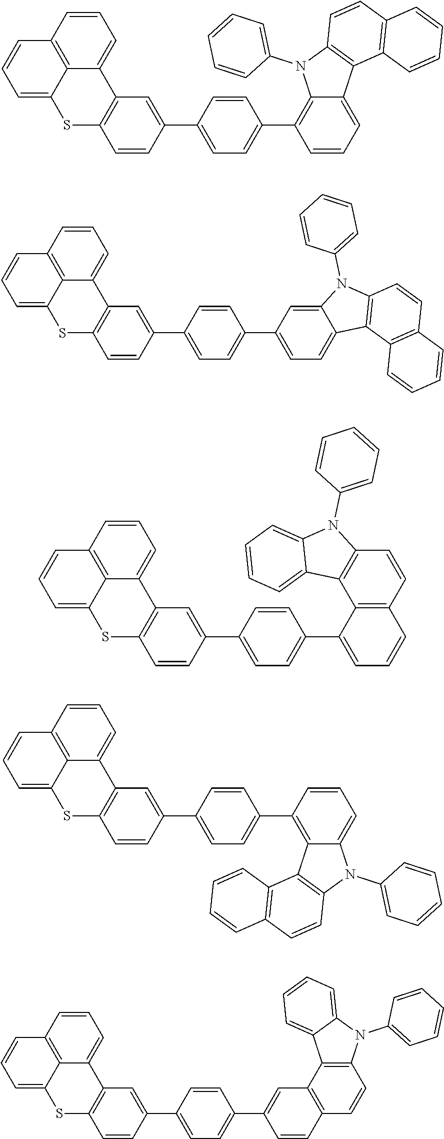

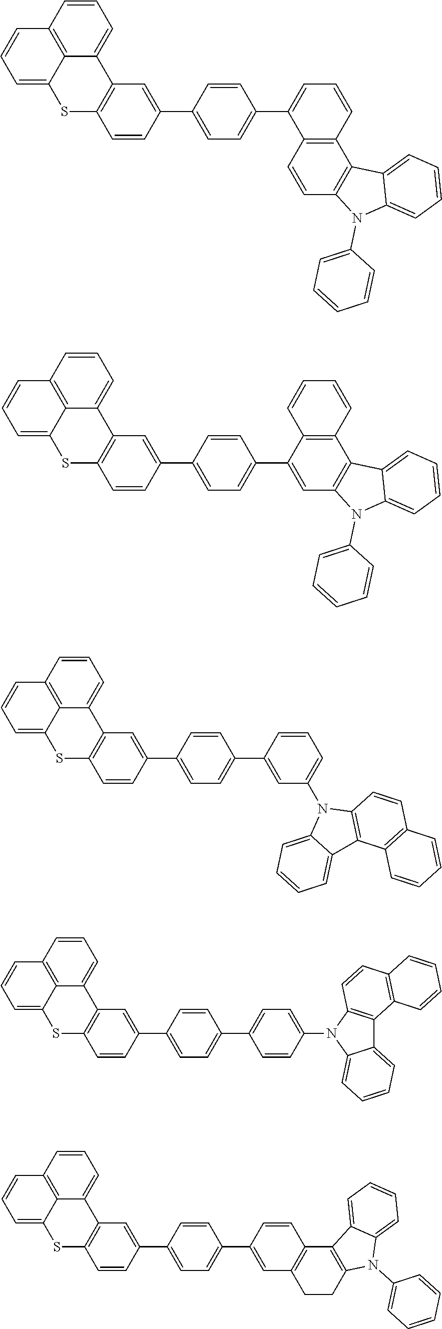 [SMILES: c1ccc(-n2c3ccccc3c3c4cccc(-c5ccc(-c6ccc7c(c6)-c6cccc8cccc(c68)S7)cc5)c4ccc32)cc1 ; c1ccc(-n2c3ccccc3c3c4ccccc4c(-c4ccc(-c5ccc6c(c5)-c5cccc7cccc(c57)S6)cc4)cc32)cc1 ; c1cc(-c2ccc(-c3ccc4c(c3)-c3cccc5cccc(c35)S4)cc2)cc(-n2c3ccccc3c3c4ccccc4ccc32)c1 ; c1cc2c3c(cccc3c1)-c1cc(-c3ccc(-c4ccc(-n5c6ccccc6c6c7ccccc7ccc65)cc4)cc3)ccc1S2 ; c1ccc(-n2c3c(c4ccccc42)-c2ccc(-c4ccc(-c5ccc6c(c5)-c5cccc7cccc(c57)S6)cc4)cc2CC3)cc1]

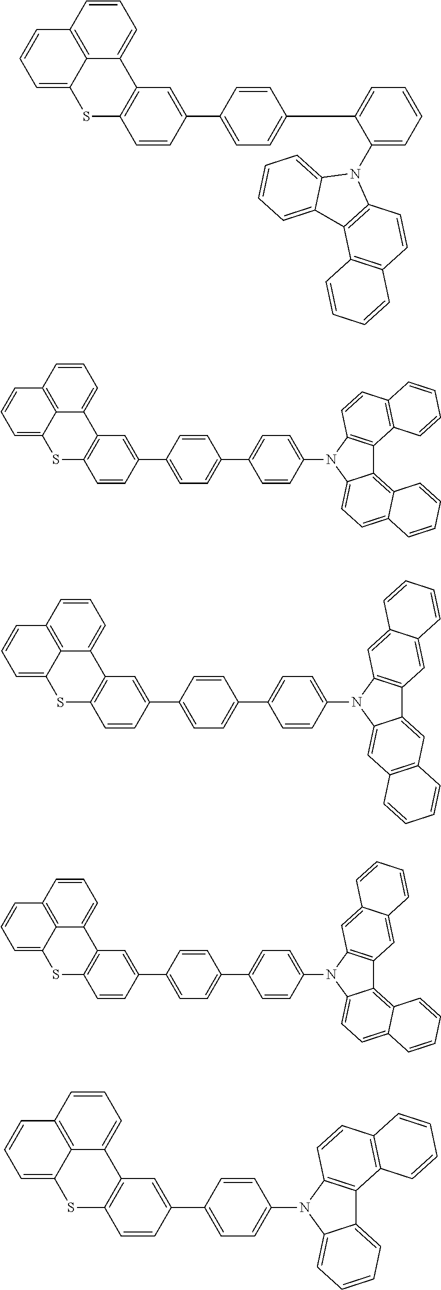 [SMILES: c1ccc(-n2c3ccccc3c3c4ccccc4ccc32)c(-c2ccc(-c3ccc4c(c3)-c3cccc5cccc(c35)S4)cc2)c1 ; c1cc2c3c(cccc3c1)-c1cc(-c3ccc(-c4ccc(-n5c6ccc7ccccc7c6c6c7ccccc7ccc65)cc4)cc3)ccc1S2 ; c1ccc2cc3c(cc2c1)c1cc2ccccc2cc1n3-c1ccc(-c2ccc(-c3ccc4c(c3)-c3cccc5cccc(c35)S4)cc2)cc1 ; c1ccc2cc3c(cc2c1)c1c2ccccc2ccc1n3-c1ccc(-c2ccc(-c3ccc4c(c3)-c3cccc5cccc(c35)S4)cc2)cc1 ; c1cc2c3c(cccc3c1)-c1cc(-c3ccc(-n4c5ccccc5c5c6ccccc6ccc54)cc3)ccc1S2]

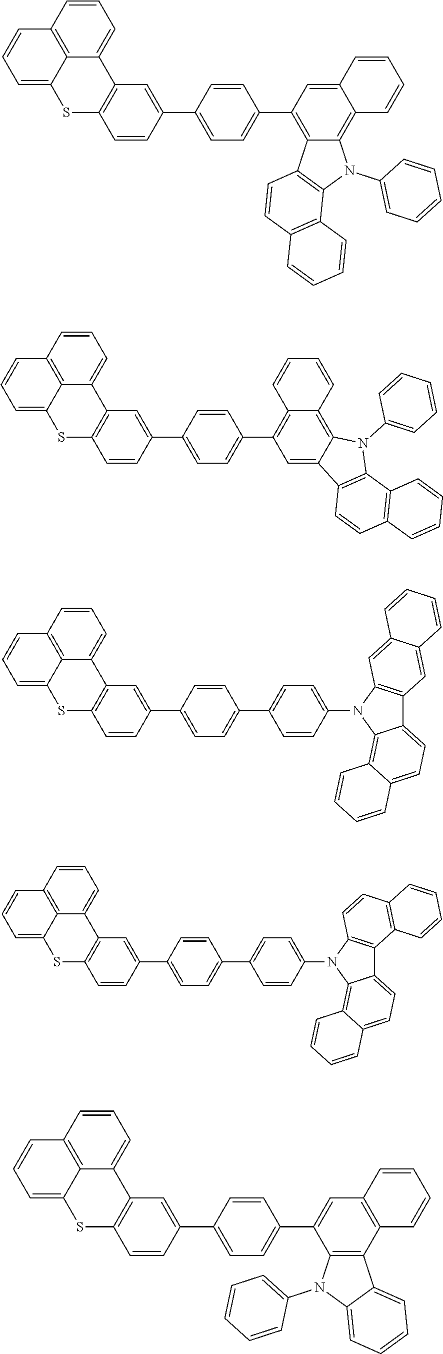 [SMILES: c1ccc(-n2c3c4ccccc4ccc3c3c(-c4ccc(-c5ccc6c(c5)-c5cccc7cccc(c57)S6)cc4)cc4ccccc4c32)cc1 ; c1ccc(-n2c3c4ccccc4ccc3c3cc(-c4ccc(-c5ccc6c(c5)-c5cccc7cccc(c57)S6)cc4)c4ccccc4c32)cc1 ; c1ccc2cc3c(cc2c1)c1ccc2ccccc2c1n3-c1ccc(-c2ccc(-c3ccc4c(c3)-c3cccc5cccc(c35)S4)cc2)cc1 ; c1cc2c3c(cccc3c1)-c1cc(-c3ccc(-c4ccc(-n5c6ccc7ccccc7c6c6ccc7ccccc7c65)cc4)cc3)ccc1S2 ; c1ccc(-n2c3ccccc3c3c4ccccc4cc(-c4ccc(-c5ccc6c(c5)-c5cccc7cccc(c57)S6)cc4)c32)cc1]

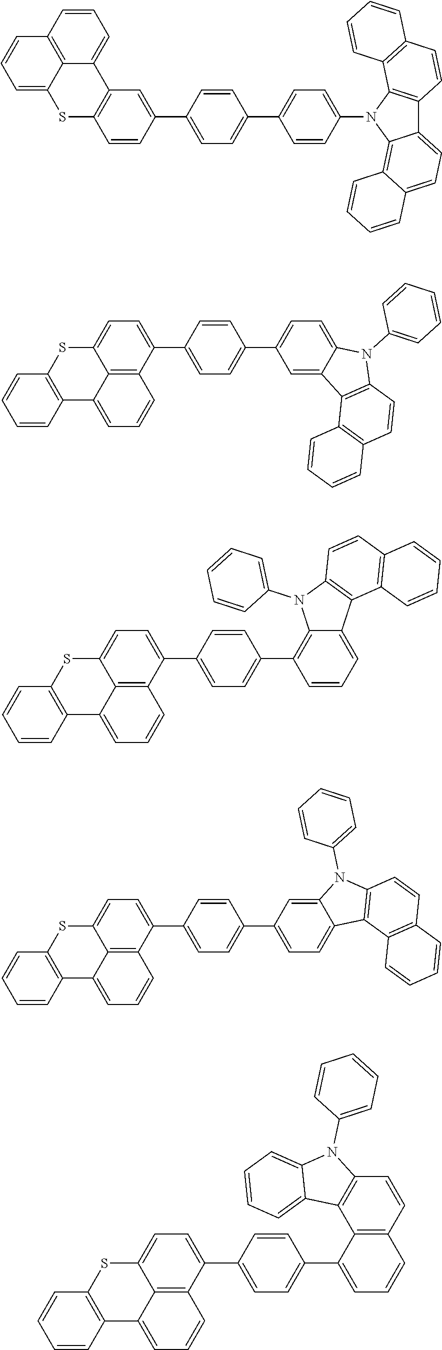 [SMILES: c1cc2c3c(cccc3c1)-c1cc(-c3ccc(-c4ccc(-n5c6c7ccccc7ccc6c6ccc7ccccc7c65)cc4)cc3)ccc1S2 ; c1ccc(-n2c3ccc(-c4ccc(-c5ccc6c7c(cccc57)-c5ccccc5S6)cc4)cc3c3c4ccccc4ccc32)cc1 ; c1ccc(-n2c3ccc4ccccc4c3c3cccc(-c4ccc(-c5ccc6c7c(cccc57)-c5ccccc5S6)cc4)c32)cc1 ; c1ccc(-n2c3cc(-c4ccc(-c5ccc6c7c(cccc57)-c5ccccc5S6)cc4)ccc3c3c4ccccc4ccc32)cc1 ; c1ccc(-n2c3ccccc3c3c4c(-c5ccc(-c6ccc7c8c(cccc68)-c6ccccc6S7)cc5)cccc4ccc32)cc1]

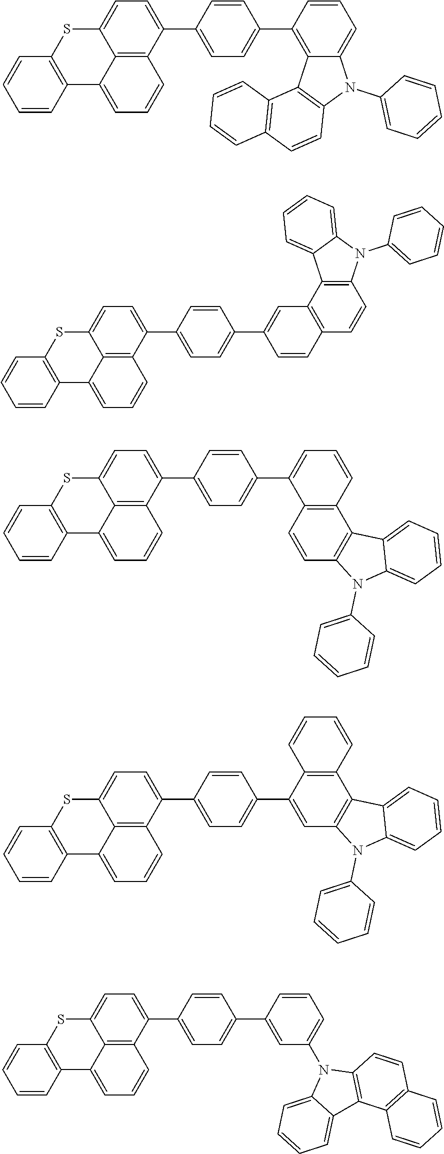

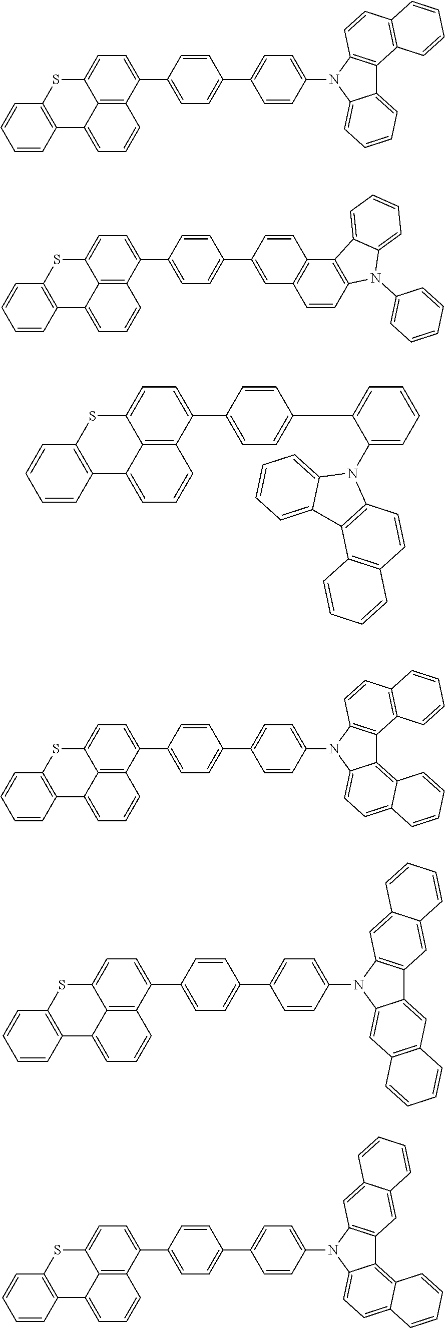

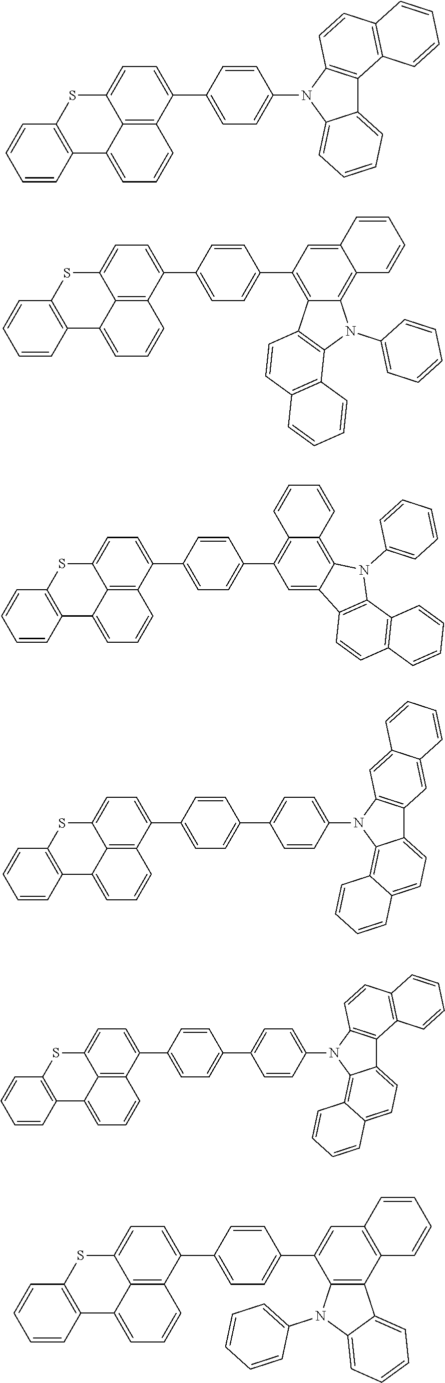

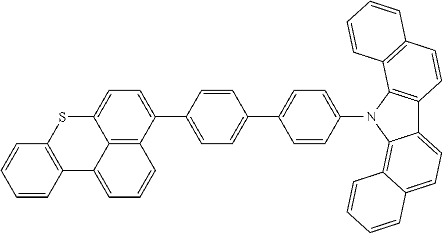 [SMILES: c1ccc2c(c1)Sc1ccc(-c3ccc(-c4ccc(-n5c6c7ccccc7ccc6c6ccc7ccccc7c65)cc4)cc3)c3cccc-2c13]

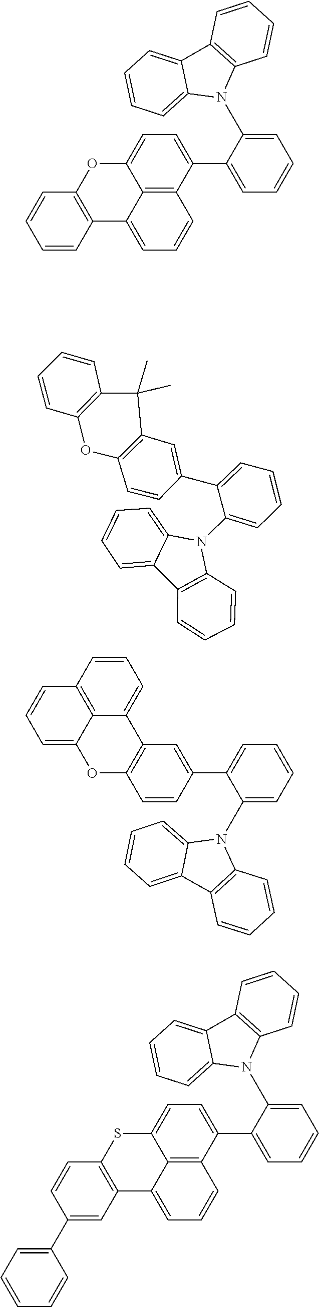

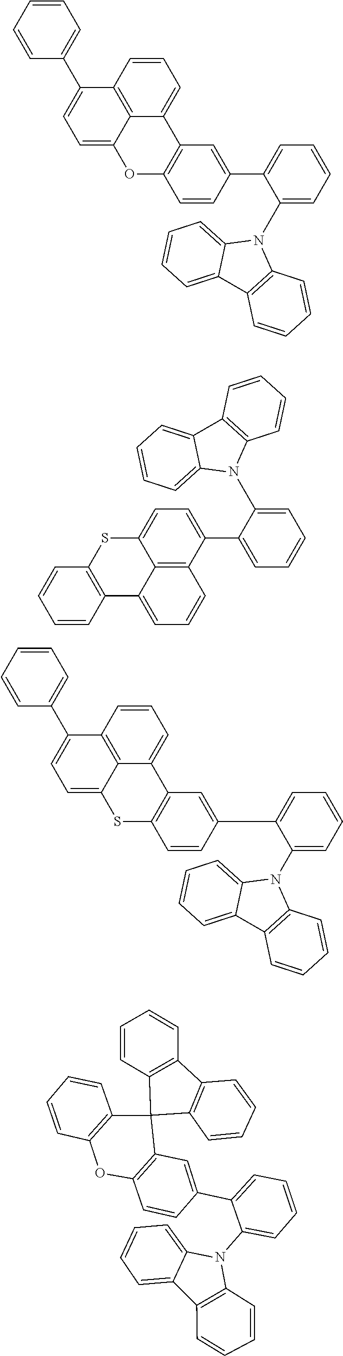

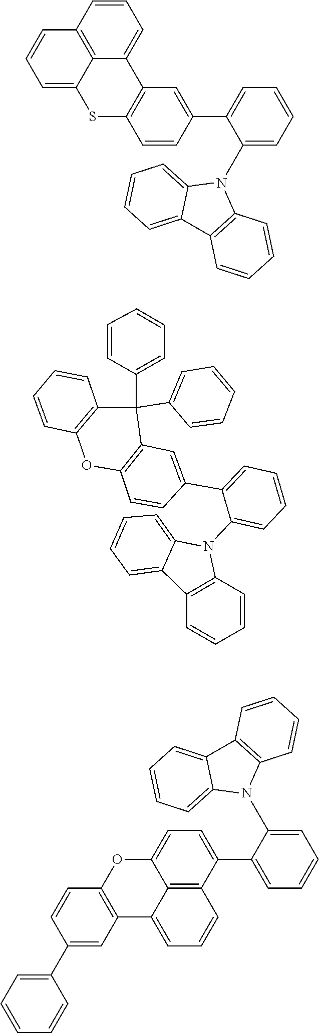 [SMILES: c1ccc(-n2c3ccccc3c3ccccc32)c(-c2ccc3c(c2)-c2cccc4cccc(c24)S3)c1 ; c1ccc(C2(c3ccccc3)c3ccccc3Oc3ccc(-c4ccccc4-n4c5ccccc5c5ccccc54)cc32)cc1 ; c1ccc(-c2ccc3c(c2)-c2cccc4c(-c5ccccc5-n5c6ccccc6c6ccccc65)ccc(c24)O3)cc1]

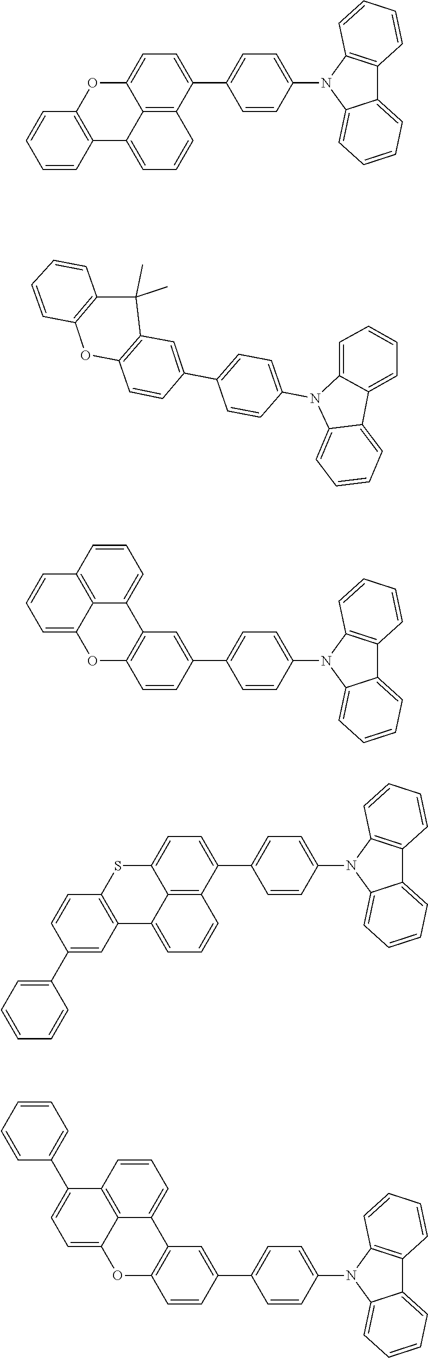 [SMILES: c1ccc2c(c1)Oc1ccc(-c3ccc(-n4c5ccccc5c5ccccc54)cc3)c3cccc-2c13 ; CC1(C)c2ccccc2Oc2ccc(-c3ccc(-n4c5ccccc5c5ccccc54)cc3)cc21 ; c1cc2c3c(cccc3c1)-c1cc(-c3ccc(-n4c5ccccc5c5ccccc54)cc3)ccc1O2 ; c1ccc(-c2ccc3c(c2)-c2cccc4c(-c5ccc(-n6c7ccccc7c7ccccc76)cc5)ccc(c24)S3)cc1 ; c1ccc(-c2ccc3c4c(cccc24)-c2cc(-c4ccc(-n5c6ccccc6c6ccccc65)cc4)ccc2O3)cc1]

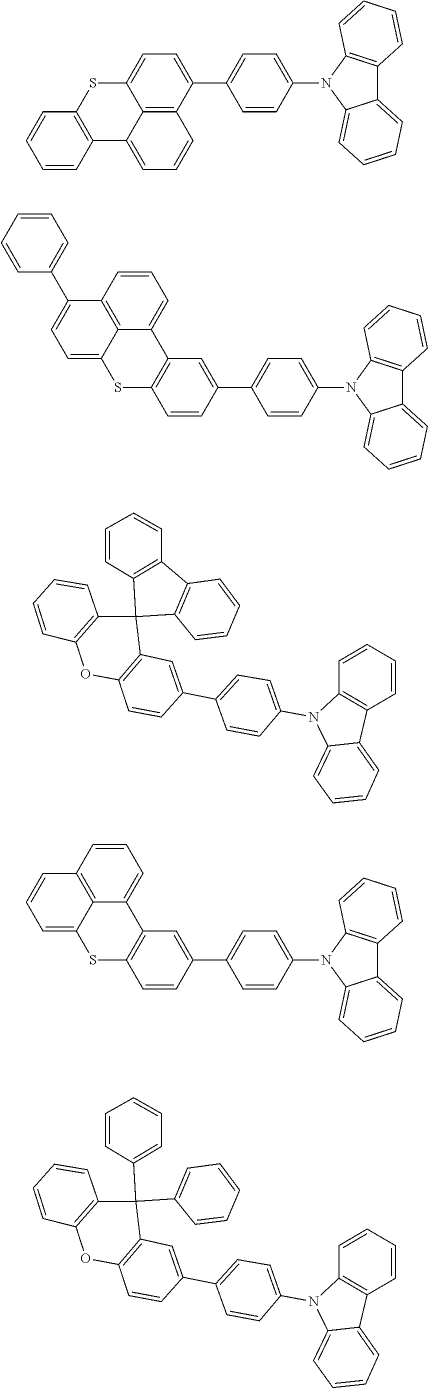

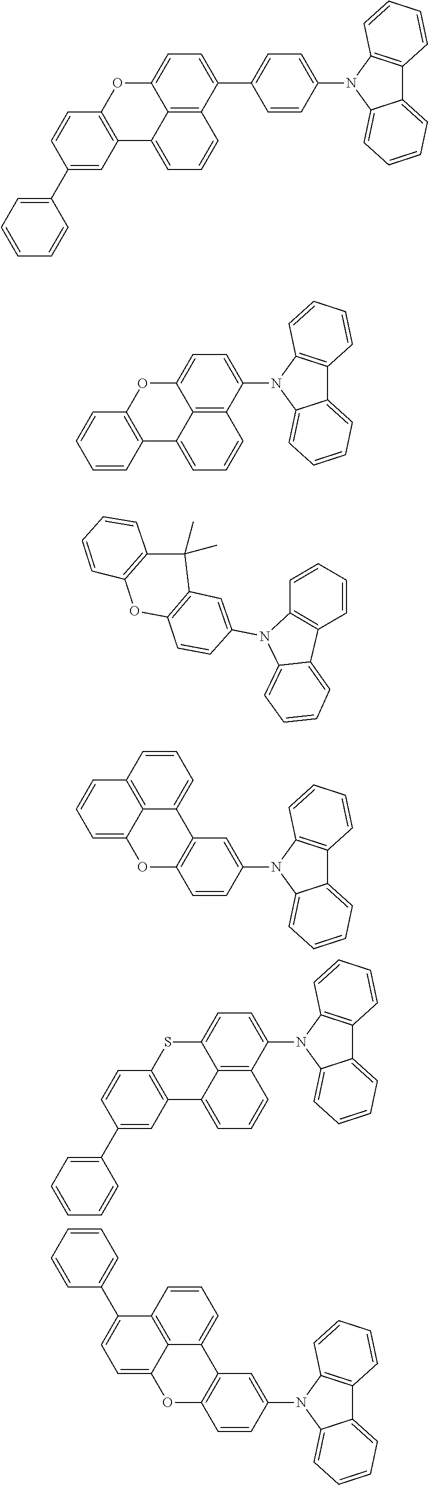

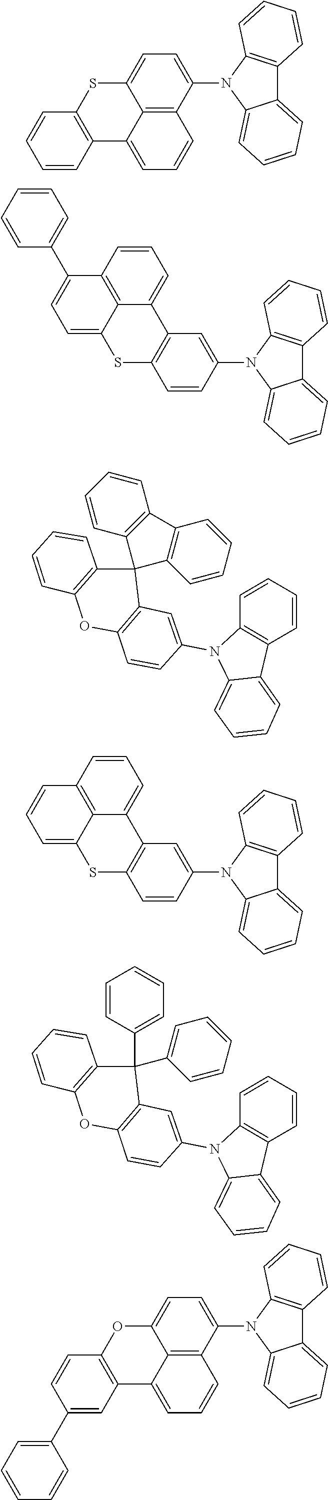

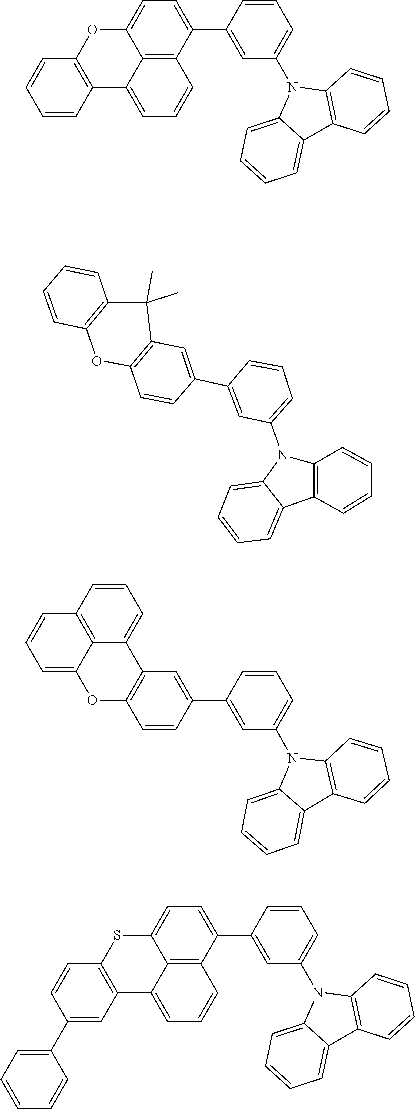 [SMILES: c1cc(-c2ccc3c4c(cccc24)-c2ccccc2O3)cc(-n2c3ccccc3c3ccccc32)c1 ; CC1(C)c2ccccc2Oc2ccc(-c3cccc(-n4c5ccccc5c5ccccc54)c3)cc21 ; c1cc(-c2ccc3c(c2)-c2cccc4cccc(c24)O3)cc(-n2c3ccccc3c3ccccc32)c1 ; c1ccc(-c2ccc3c(c2)-c2cccc4c(-c5cccc(-n6c7ccccc7c7ccccc76)c5)ccc(c24)S3)cc1]

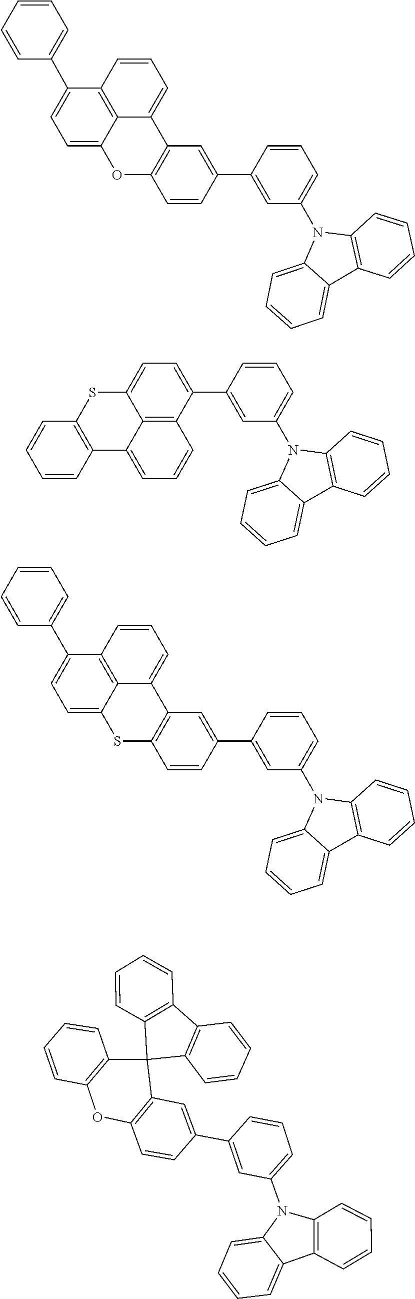

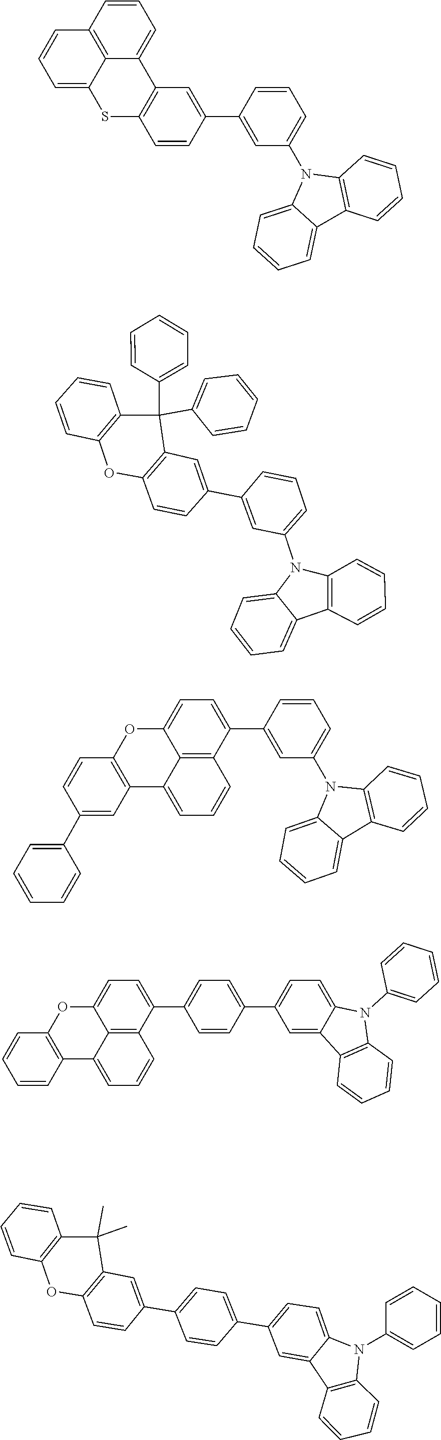 [SMILES: c1cc(-c2ccc3c(c2)-c2cccc4cccc(c24)S3)cc(-n2c3ccccc3c3ccccc32)c1 ; c1ccc(C2(c3ccccc3)c3ccccc3Oc3ccc(-c4cccc(-n5c6ccccc6c6ccccc65)c4)cc32)cc1 ; c1ccc(-c2ccc3c(c2)-c2cccc4c(-c5cccc(-n6c7ccccc7c7ccccc76)c5)ccc(c24)O3)cc1 ; c1ccc(-n2c3ccccc3c3cc(-c4ccc(-c5ccc6c7c(cccc57)-c5ccccc5O6)cc4)ccc32)cc1 ; CC1(C)c2ccccc2Oc2ccc(-c3ccc(-c4ccc5c(c4)c4ccccc4n5-c4ccccc4)cc3)cc21]

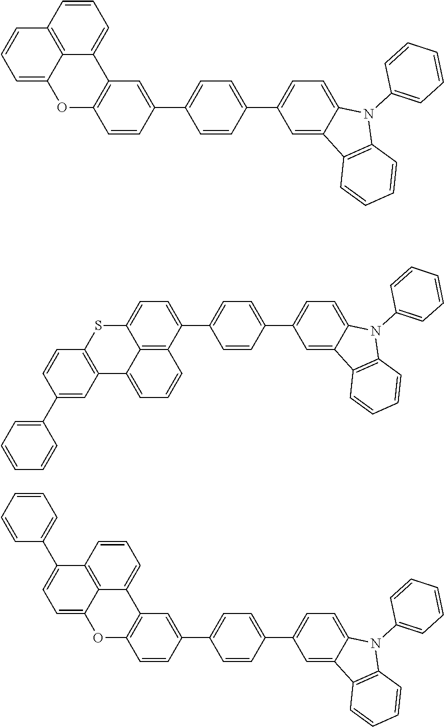

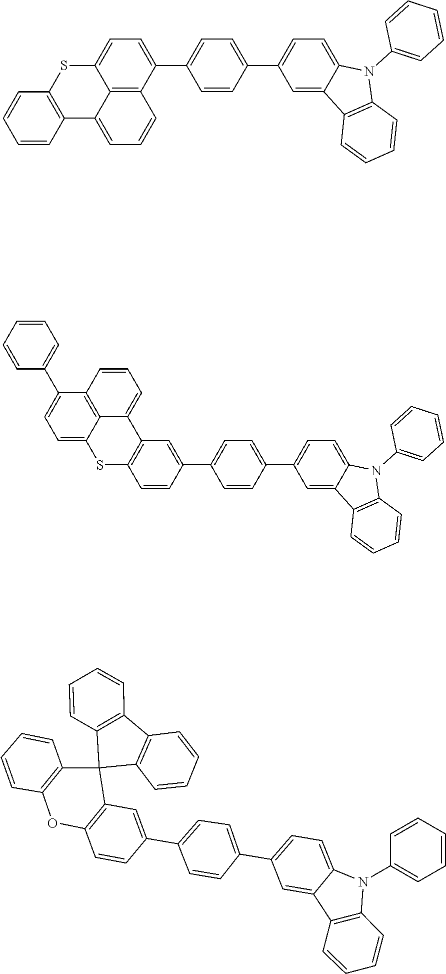 [SMILES: c1ccc(-n2c3ccccc3c3cc(-c4ccc(-c5ccc6c7c(cccc57)-c5ccccc5S6)cc4)ccc32)cc1 ; c1ccc(-c2ccc3c4c(cccc24)-c2cc(-c4ccc(-c5ccc6c(c5)c5ccccc5n6-c5ccccc5)cc4)ccc2S3)cc1 ; c1ccc(-n2c3ccccc3c3cc(-c4ccc(-c5ccc6c(c5)C5(c7ccccc7O6)c6ccccc6-c6ccccc65)cc4)ccc32)cc1]

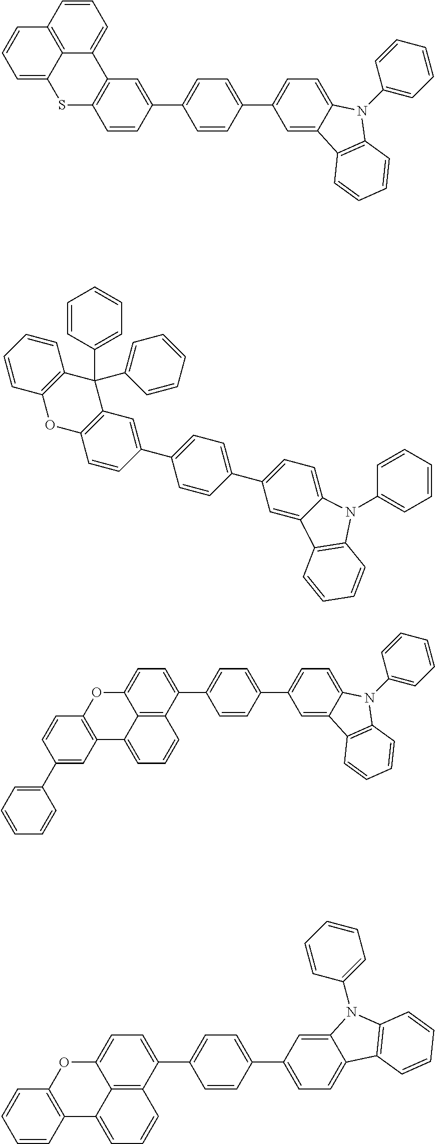 [SMILES: c1ccc(-n2c3ccccc3c3cc(-c4ccc(-c5ccc6c(c5)-c5cccc7cccc(c57)S6)cc4)ccc32)cc1 ; c1ccc(-n2c3ccccc3c3cc(-c4ccc(-c5ccc6c(c5)C(c5ccccc5)(c5ccccc5)c5ccccc5O6)cc4)ccc32)cc1 ; c1ccc(-c2ccc3c(c2)-c2cccc4c(-c5ccc(-c6ccc7c(c6)c6ccccc6n7-c6ccccc6)cc5)ccc(c24)O3)cc1 ; c1ccc(-n2c3ccccc3c3ccc(-c4ccc(-c5ccc6c7c(cccc57)-c5ccccc5O6)cc4)cc32)cc1]

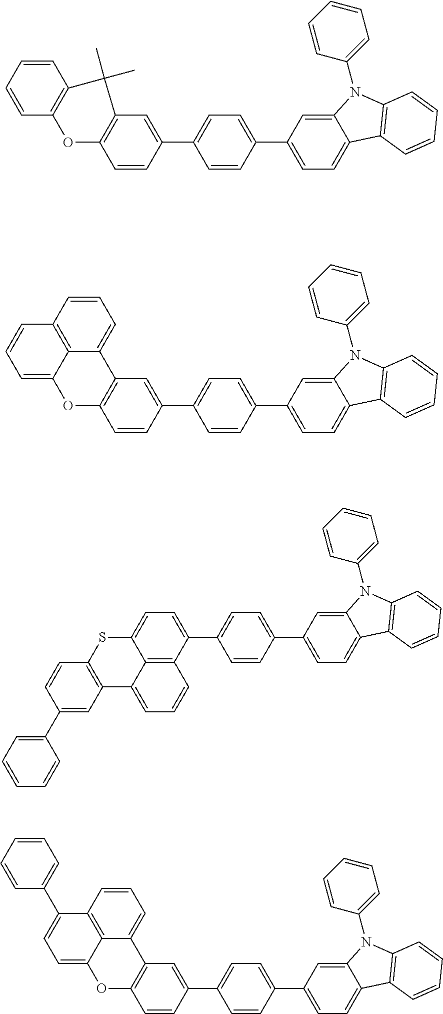 [SMILES: CC1(C)c2ccccc2Oc2ccc(-c3ccc(-c4ccc5c6ccccc6n(-c6ccccc6)c5c4)cc3)cc21 ; c1ccc(-n2c3ccccc3c3ccc(-c4ccc(-c5ccc6c(c5)-c5cccc7cccc(c57)O6)cc4)cc32)cc1 ; c1ccc(-c2ccc3c(c2)-c2cccc4c(-c5ccc(-c6ccc7c8ccccc8n(-c8ccccc8)c7c6)cc5)ccc(c24)S3)cc1 ; c1ccc(-c2ccc3c4c(cccc24)-c2cc(-c4ccc(-c5ccc6c7ccccc7n(-c7ccccc7)c6c5)cc4)ccc2O3)cc1]

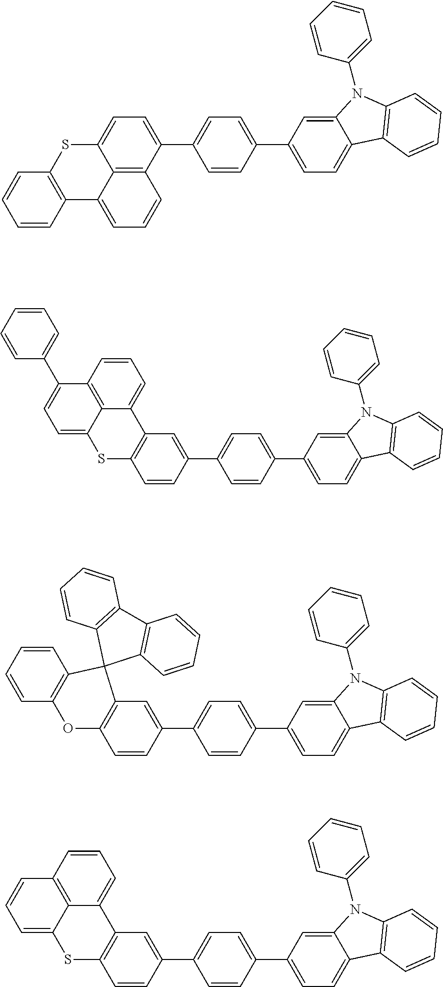 [SMILES: c1ccc(-n2c3ccccc3c3ccc(-c4ccc(-c5ccc6c7c(cccc57)-c5ccccc5S6)cc4)cc32)cc1 ; c1ccc(-c2ccc3c4c(cccc24)-c2cc(-c4ccc(-c5ccc6c7ccccc7n(-c7ccccc7)c6c5)cc4)ccc2S3)cc1 ; c1ccc(-n2c3ccccc3c3ccc(-c4ccc(-c5ccc6c(c5)C5(c7ccccc7O6)c6ccccc6-c6ccccc65)cc4)cc32)cc1 ; c1ccc(-n2c3ccccc3c3ccc(-c4ccc(-c5ccc6c(c5)-c5cccc7cccc(c57)S6)cc4)cc32)cc1]

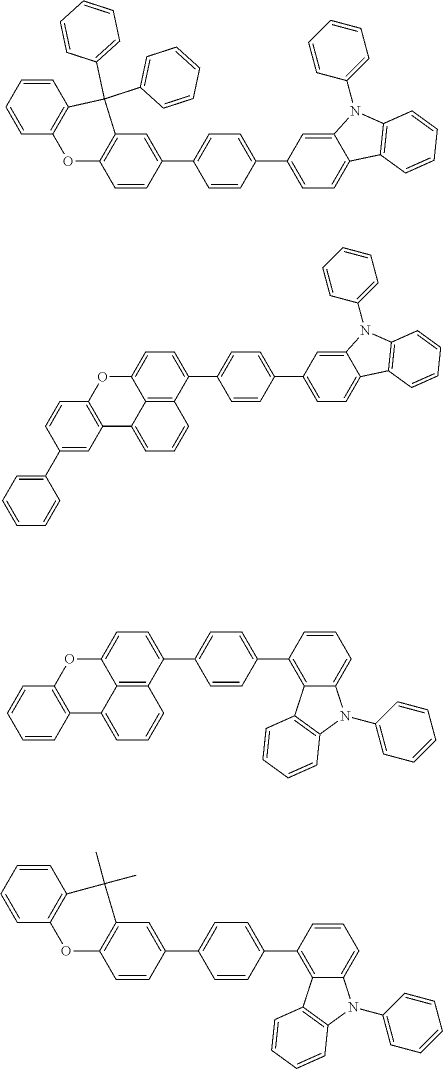 [SMILES: c1ccc(-n2c3ccccc3c3ccc(-c4ccc(-c5ccc6c(c5)C(c5ccccc5)(c5ccccc5)c5ccccc5O6)cc4)cc32)cc1 ; c1ccc(-c2ccc3c(c2)-c2cccc4c(-c5ccc(-c6ccc7c8ccccc8n(-c8ccccc8)c7c6)cc5)ccc(c24)O3)cc1 ; c1ccc(-n2c3ccccc3c3c(-c4ccc(-c5ccc6c7c(cccc57)-c5ccccc5O6)cc4)cccc32)cc1 ; CC1(C)c2ccccc2Oc2ccc(-c3ccc(-c4cccc5c4c4ccccc4n5-c4ccccc4)cc3)cc21]

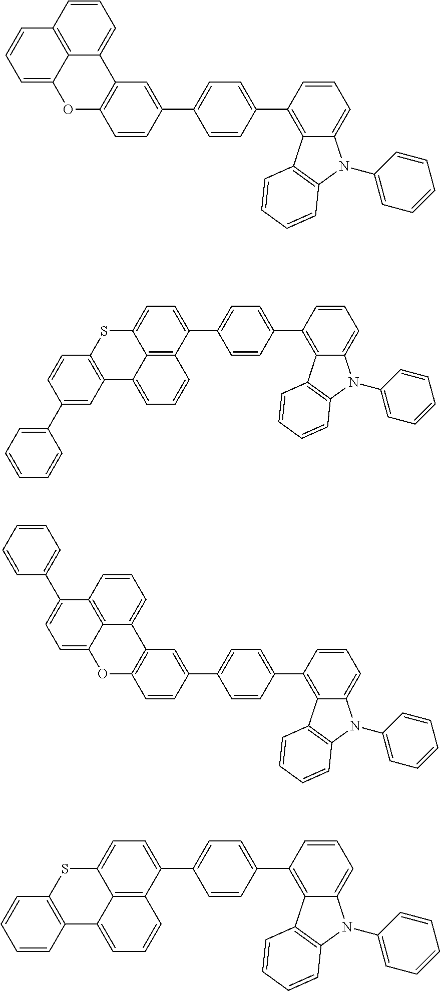 [SMILES: c1ccc(-n2c3ccccc3c3c(-c4ccc(-c5ccc6c(c5)-c5cccc7cccc(c57)O6)cc4)cccc32)cc1 ; c1ccc(-c2ccc3c(c2)-c2cccc4c(-c5ccc(-c6cccc7c6c6ccccc6n7-c6ccccc6)cc5)ccc(c24)S3)cc1 ; c1ccc(-c2ccc3c4c(cccc24)-c2cc(-c4ccc(-c5cccc6c5c5ccccc5n6-c5ccccc5)cc4)ccc2O3)cc1 ; c1ccc(-n2c3ccccc3c3c(-c4ccc(-c5ccc6c7c(cccc57)-c5ccccc5S6)cc4)cccc32)cc1]

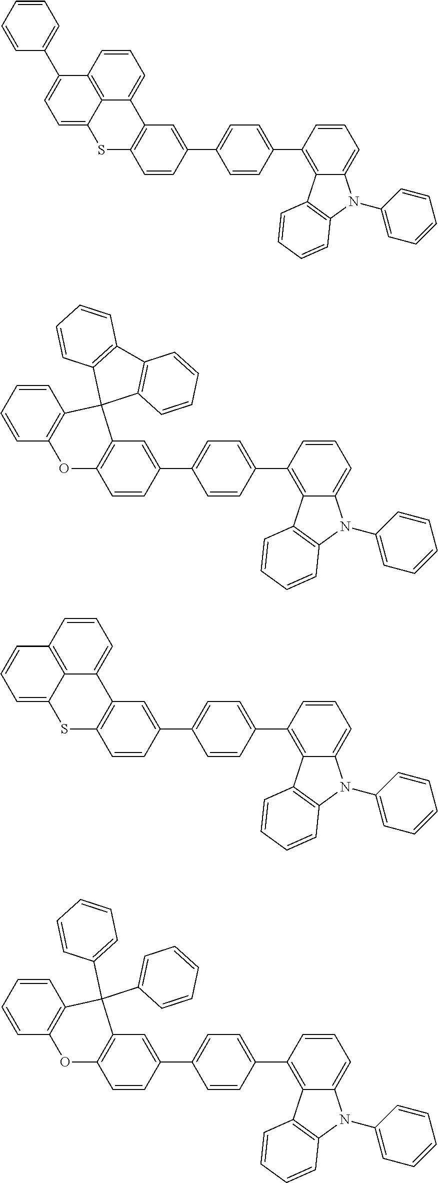 [SMILES: c1ccc(-c2ccc3c4c(cccc24)-c2cc(-c4ccc(-c5cccc6c5c5ccccc5n6-c5ccccc5)cc4)ccc2S3)cc1 ; c1ccc(-n2c3ccccc3c3c(-c4ccc(-c5ccc6c(c5)C5(c7ccccc7O6)c6ccccc6-c6ccccc65)cc4)cccc32)cc1 ; c1ccc(-n2c3ccccc3c3c(-c4ccc(-c5ccc6c(c5)-c5cccc7cccc(c57)S6)cc4)cccc32)cc1 ; c1ccc(-n2c3ccccc3c3c(-c4ccc(-c5ccc6c(c5)C(c5ccccc5)(c5ccccc5)c5ccccc5O6)cc4)cccc32)cc1]

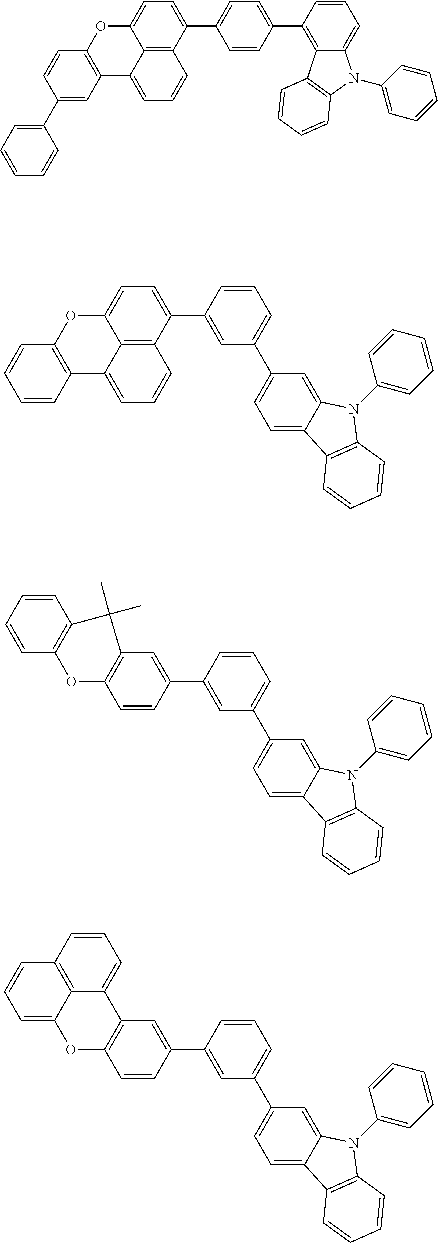 [SMILES: c1ccc(-c2ccc3c(c2)-c2cccc4c(-c5ccc(-c6cccc7c6c6ccccc6n7-c6ccccc6)cc5)ccc(c24)O3)cc1 ; c1ccc(-n2c3ccccc3c3ccc(-c4cccc(-c5ccc6c7c(cccc57)-c5ccccc5O6)c4)cc32)cc1 ; CC1(C)c2ccccc2Oc2ccc(-c3cccc(-c4ccc5c6ccccc6n(-c6ccccc6)c5c4)c3)cc21 ; c1ccc(-n2c3ccccc3c3ccc(-c4cccc(-c5ccc6c(c5)-c5cccc7cccc(c57)O6)c4)cc32)cc1]

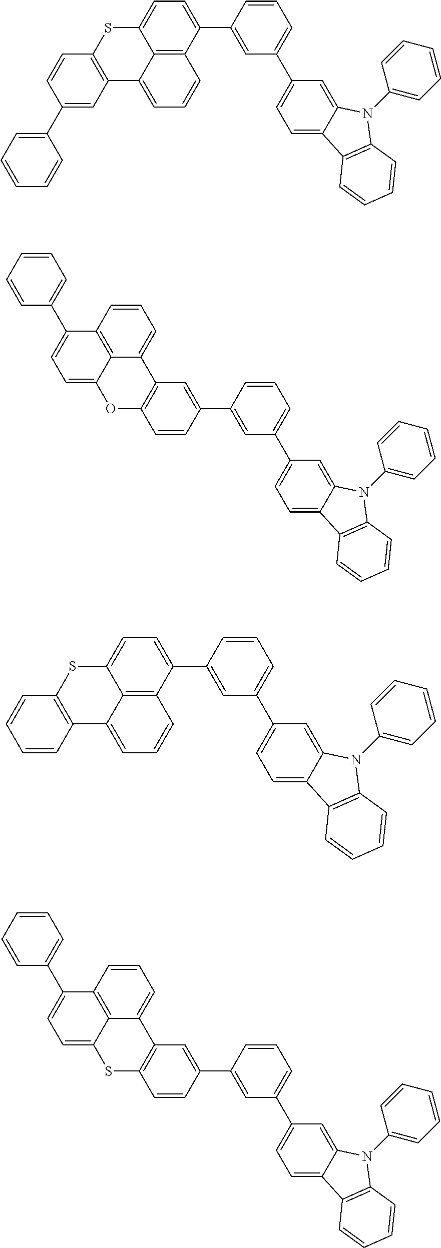 [SMILES: c1ccc(-c2ccc3c(c2)-c2cccc4c(-c5cccc(-c6ccc7c8ccccc8n(-c8ccccc8)c7c6)c5)ccc(c24)S3)cc1 ; c1ccc(-c2ccc3c4c(cccc24)-c2cc(-c4cccc(-c5ccc6c7ccccc7n(-c7ccccc7)c6c5)c4)ccc2O3)cc1 ; c1ccc(-n2c3ccccc3c3ccc(-c4cccc(-c5ccc6c7c(cccc57)-c5ccccc5S6)c4)cc32)cc1 ; c1ccc(-c2ccc3c4c(cccc24)-c2cc(-c4cccc(-c5ccc6c7ccccc7n(-c7ccccc7)c6c5)c4)ccc2S3)cc1]

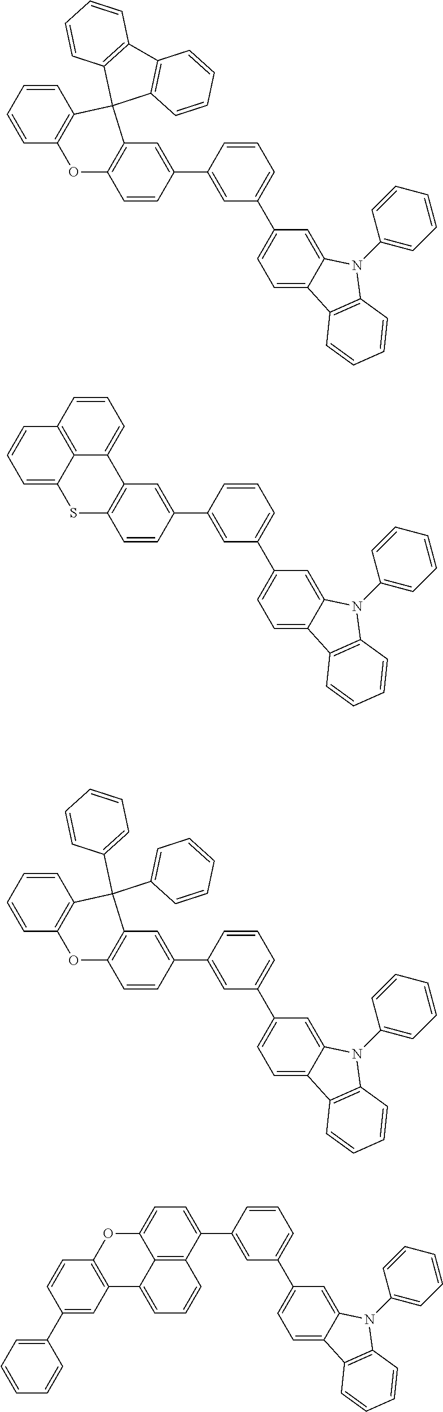 [SMILES: c1ccc(-n2c3ccccc3c3ccc(-c4cccc(-c5ccc6c(c5)C5(c7ccccc7O6)c6ccccc6-c6ccccc65)c4)cc32)cc1 ; c1ccc(-n2c3ccccc3c3ccc(-c4cccc(-c5ccc6c(c5)-c5cccc7cccc(c57)S6)c4)cc32)cc1 ; c1ccc(-n2c3ccccc3c3ccc(-c4cccc(-c5ccc6c(c5)C(c5ccccc5)(c5ccccc5)c5ccccc5O6)c4)cc32)cc1 ; c1ccc(-c2ccc3c(c2)-c2cccc4c(-c5cccc(-c6ccc7c8ccccc8n(-c8ccccc8)c7c6)c5)ccc(c24)O3)cc1]

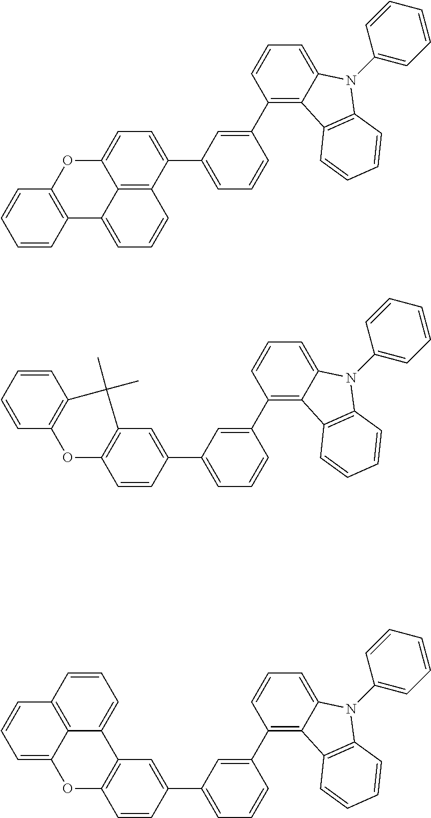 [SMILES: c1ccc(-n2c3ccccc3c3c(-c4cccc(-c5ccc6c7c(cccc57)-c5ccccc5O6)c4)cccc32)cc1 ; CC1(C)c2ccccc2Oc2ccc(-c3cccc(-c4cccc5c4c4ccccc4n5-c4ccccc4)c3)cc21 ; c1ccc(-n2c3ccccc3c3c(-c4cccc(-c5ccc6c(c5)-c5cccc7cccc(c57)O6)c4)cccc32)cc1]

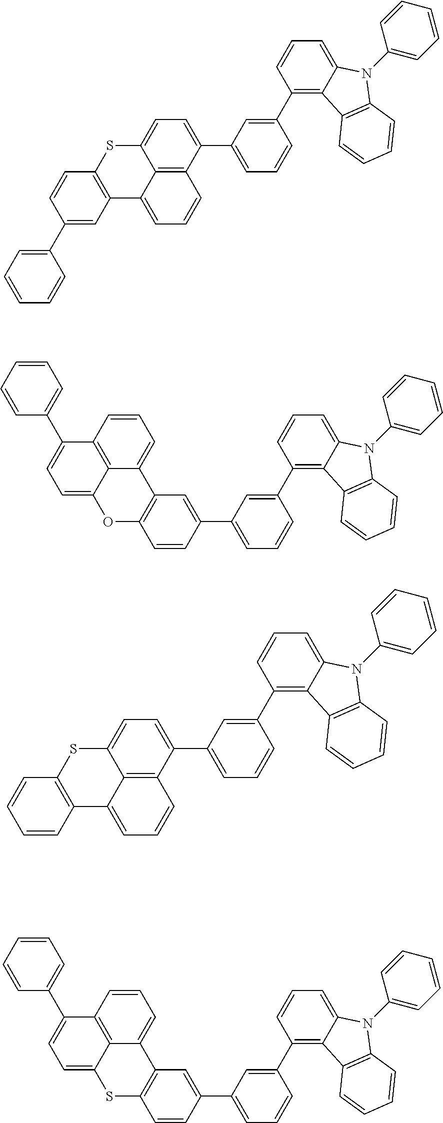 [SMILES: c1ccc(-c2ccc3c(c2)-c2cccc4c(-c5cccc(-c6cccc7c6c6ccccc6n7-c6ccccc6)c5)ccc(c24)S3)cc1 ; c1ccc(-c2ccc3c4c(cccc24)-c2cc(-c4cccc(-c5cccc6c5c5ccccc5n6-c5ccccc5)c4)ccc2O3)cc1 ; c1ccc(-n2c3ccccc3c3c(-c4cccc(-c5ccc6c7c(cccc57)-c5ccccc5S6)c4)cccc32)cc1 ; c1ccc(-c2ccc3c4c(cccc24)-c2cc(-c4cccc(-c5cccc6c5c5ccccc5n6-c5ccccc5)c4)ccc2S3)cc1]

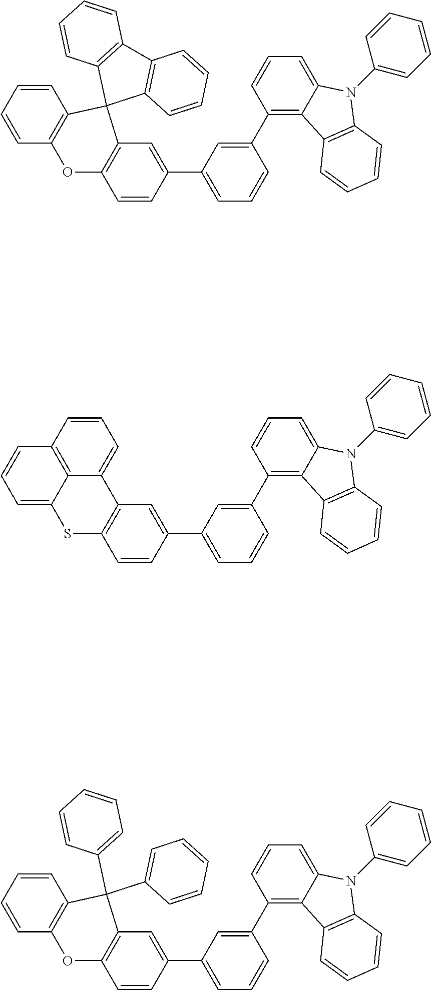 [SMILES: c1ccc(-n2c3ccccc3c3c(-c4cccc(-c5ccc6c(c5)C5(c7ccccc7O6)c6ccccc6-c6ccccc65)c4)cccc32)cc1 ; c1ccc(-n2c3ccccc3c3c(-c4cccc(-c5ccc6c(c5)-c5cccc7cccc(c57)S6)c4)cccc32)cc1 ; c1ccc(-n2c3ccccc3c3c(-c4cccc(-c5ccc6c(c5)C(c5ccccc5)(c5ccccc5)c5ccccc5O6)c4)cccc32)cc1]

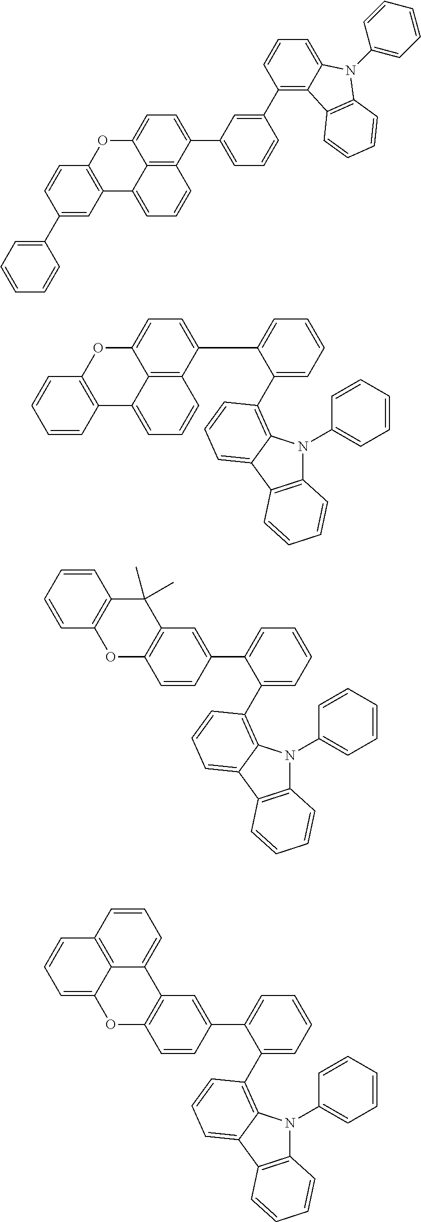 [SMILES: c1ccc(-c2ccc3c(c2)-c2cccc4c(-c5cccc(-c6cccc7c6c6ccccc6n7-c6ccccc6)c5)ccc(c24)O3)cc1 ; c1ccc(-n2c3ccccc3c3cccc(-c4ccccc4-c4ccc5c6c(cccc46)-c4ccccc4O5)c32)cc1 ; CC1(C)c2ccccc2Oc2ccc(-c3ccccc3-c3cccc4c5ccccc5n(-c5ccccc5)c34)cc21 ; c1ccc(-n2c3ccccc3c3cccc(-c4ccccc4-c4ccc5c(c4)-c4cccc6cccc(c46)O5)c32)cc1]

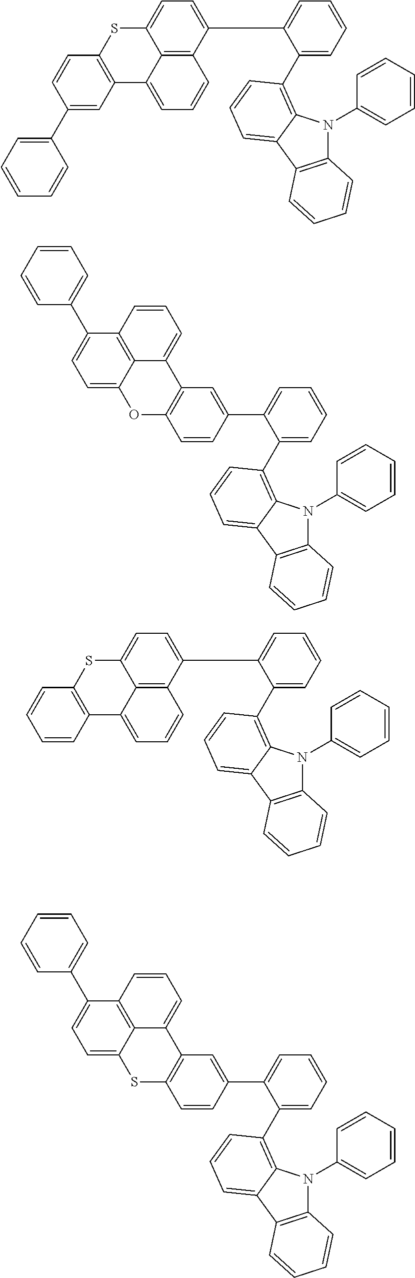

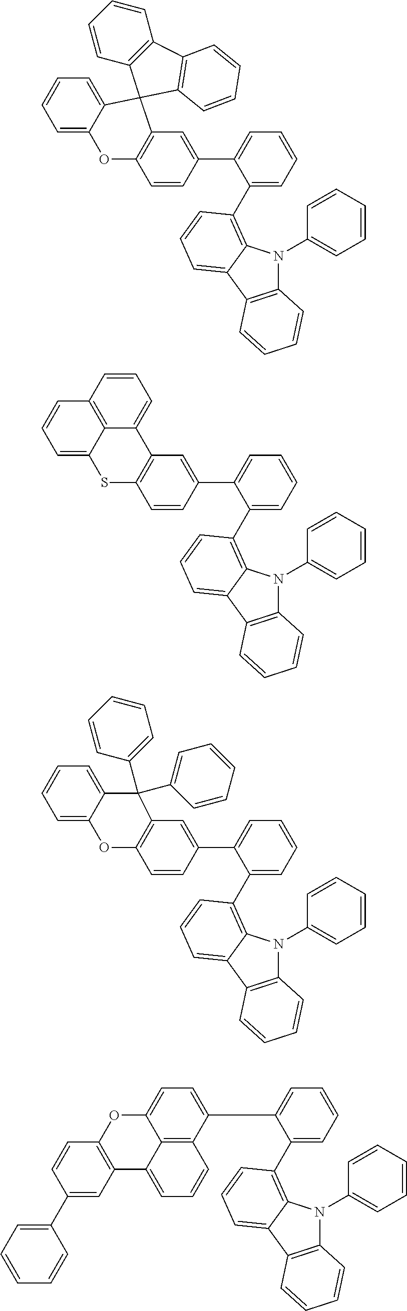 [SMILES: c1ccc(-n2c3ccccc3c3cccc(-c4ccccc4-c4ccc5c(c4)C4(c6ccccc6O5)c5ccccc5-c5ccccc54)c32)cc1 ; c1ccc(-n2c3ccccc3c3cccc(-c4ccccc4-c4ccc5c(c4)-c4cccc6cccc(c46)S5)c32)cc1 ; c1ccc(-n2c3ccccc3c3cccc(-c4ccccc4-c4ccc5c(c4)C(c4ccccc4)(c4ccccc4)c4ccccc4O5)c32)cc1 ; c1ccc(-c2ccc3c(c2)-c2cccc4c(-c5ccccc5-c5cccc6c7ccccc7n(-c7ccccc7)c56)ccc(c24)O3)cc1]

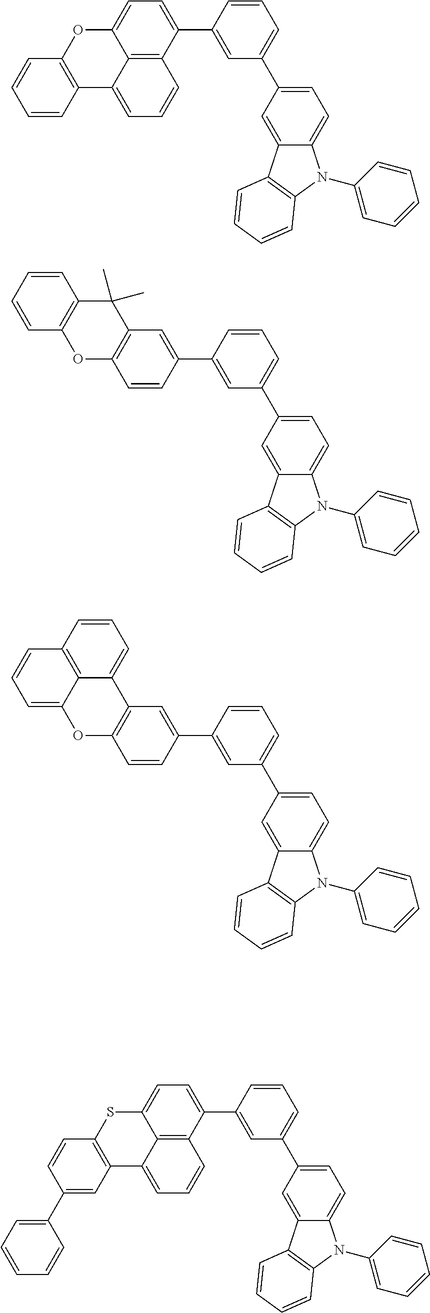 [SMILES: c1ccc(-n2c3ccccc3c3cc(-c4cccc(-c5ccc6c7c(cccc57)-c5ccccc5O6)c4)ccc32)cc1 ; CC1(C)c2ccccc2Oc2ccc(-c3cccc(-c4ccc5c(c4)c4ccccc4n5-c4ccccc4)c3)cc21 ; c1ccc(-n2c3ccccc3c3cc(-c4cccc(-c5ccc6c(c5)-c5cccc7cccc(c57)O6)c4)ccc32)cc1 ; c1ccc(-c2ccc3c(c2)-c2cccc4c(-c5cccc(-c6ccc7c(c6)c6ccccc6n7-c6ccccc6)c5)ccc(c24)S3)cc1]

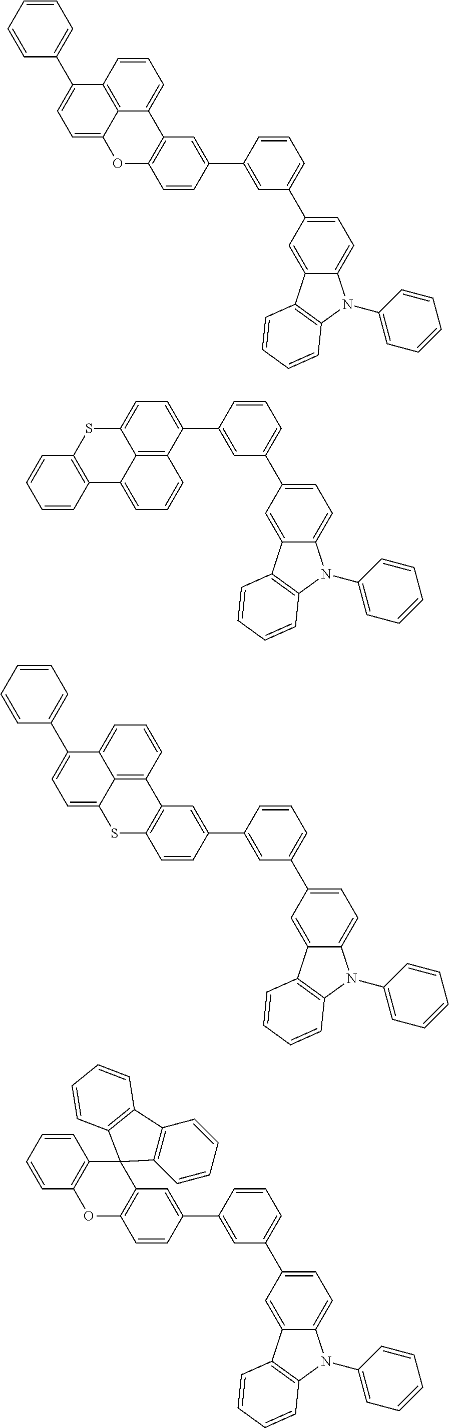

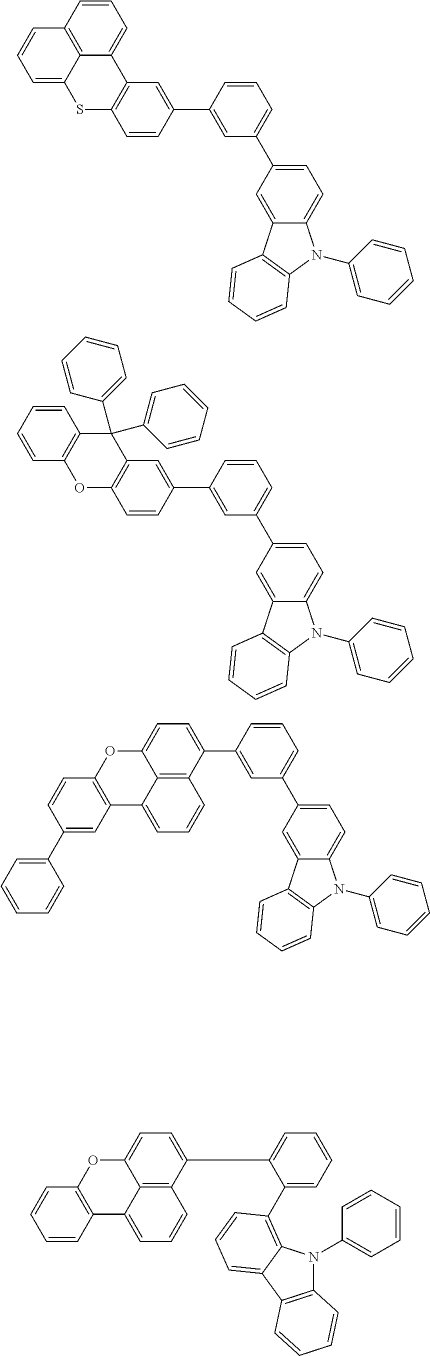

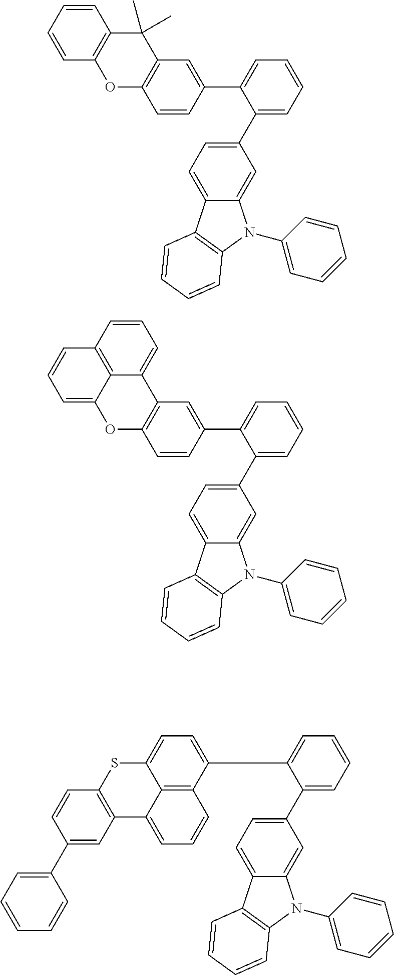

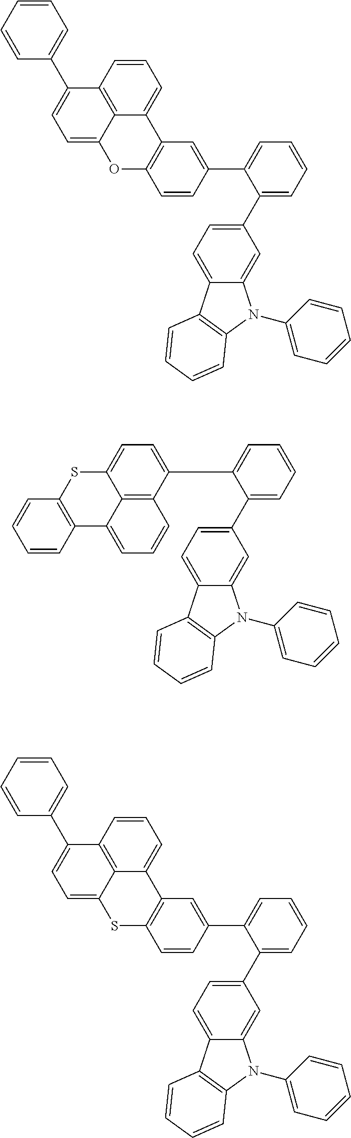 [SMILES: c1ccc(-c2ccc3c4c(cccc24)-c2cc(-c4ccccc4-c4ccc5c6ccccc6n(-c6ccccc6)c5c4)ccc2O3)cc1 ; c1ccc(-n2c3ccccc3c3ccc(-c4ccccc4-c4ccc5c6c(cccc46)-c4ccccc4S5)cc32)cc1 ; c1ccc(-c2ccc3c4c(cccc24)-c2cc(-c4ccccc4-c4ccc5c6ccccc6n(-c6ccccc6)c5c4)ccc2S3)cc1]

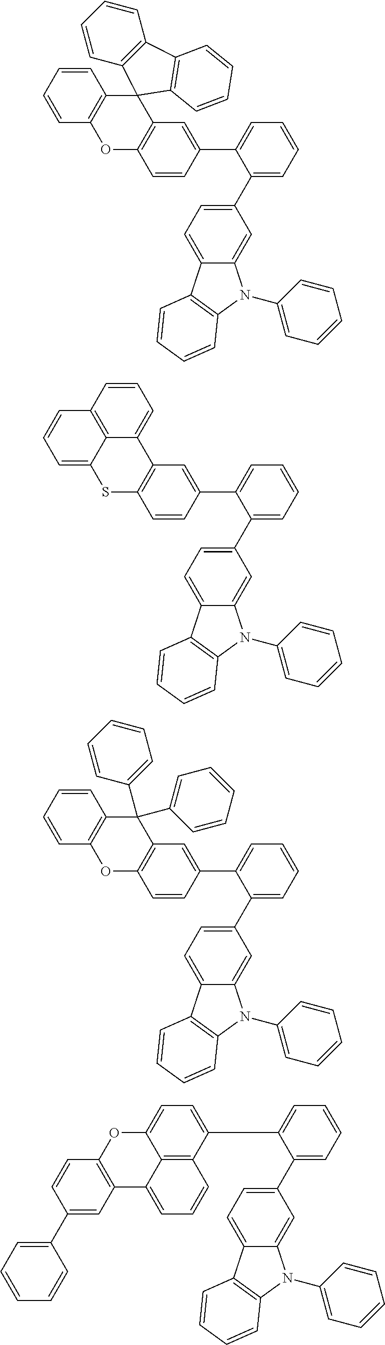

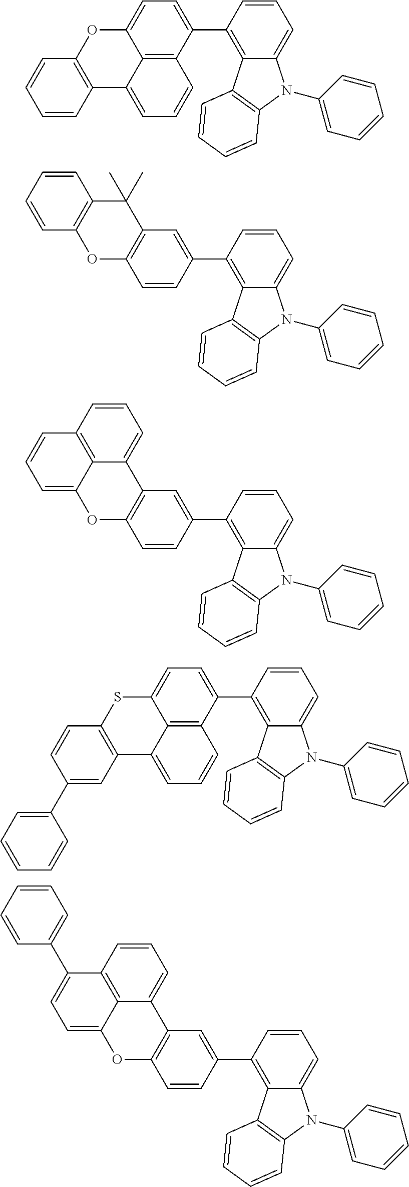

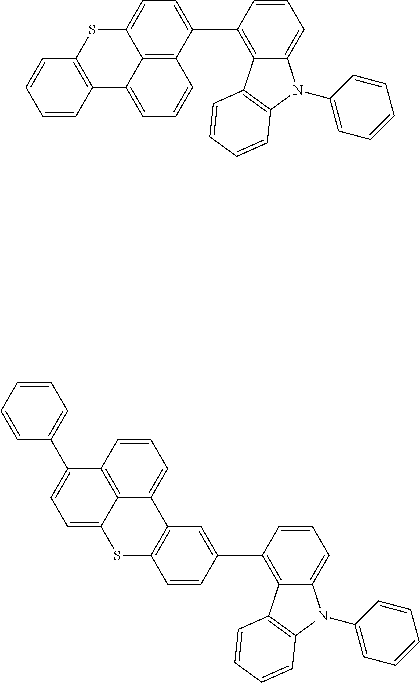 [SMILES: c1ccc(-n2c3ccccc3c3c(-c4ccc5c6c(cccc46)-c4ccccc4S5)cccc32)cc1 ; c1ccc(-c2ccc3c4c(cccc24)-c2cc(-c4cccc5c4c4ccccc4n5-c4ccccc4)ccc2S3)cc1]

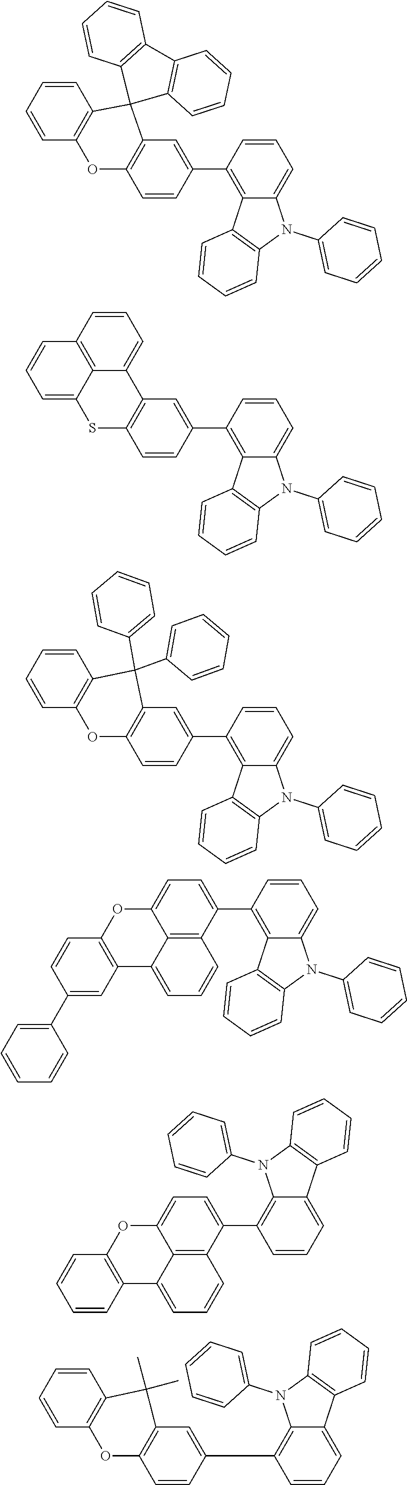

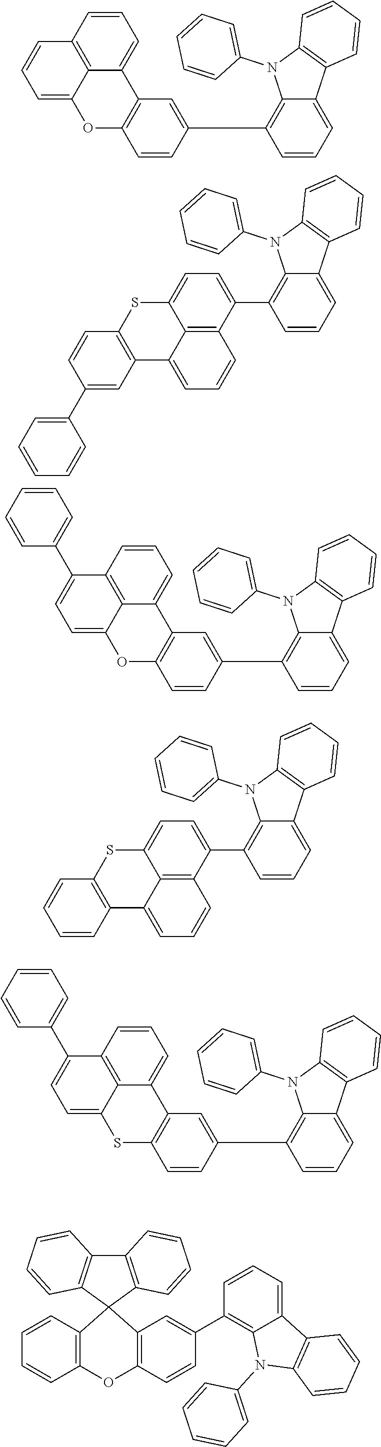

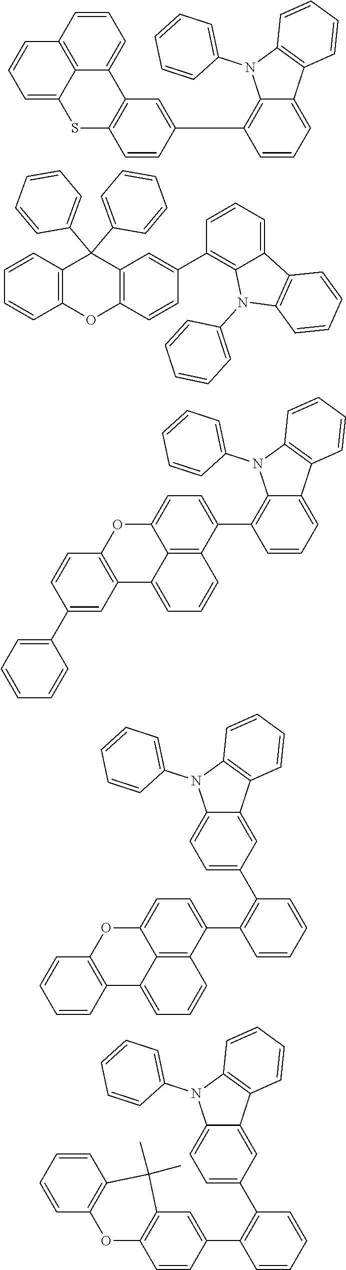

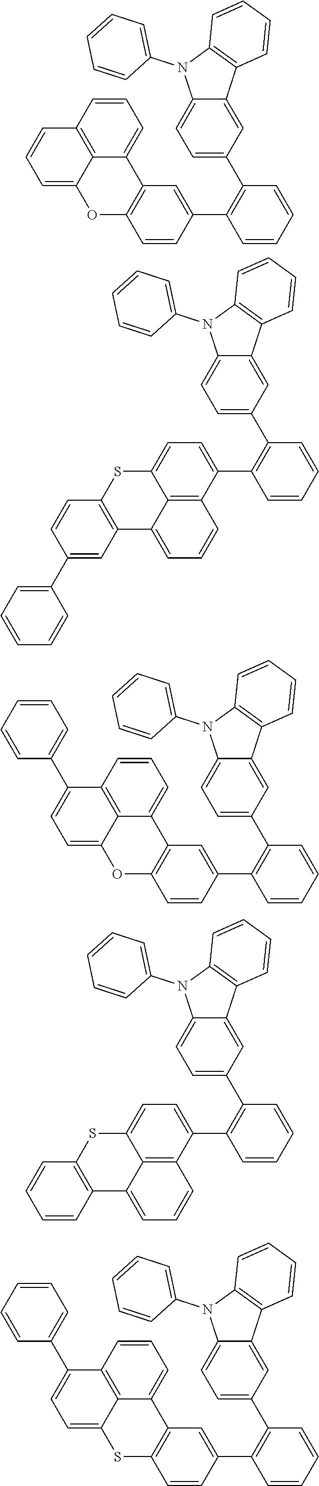

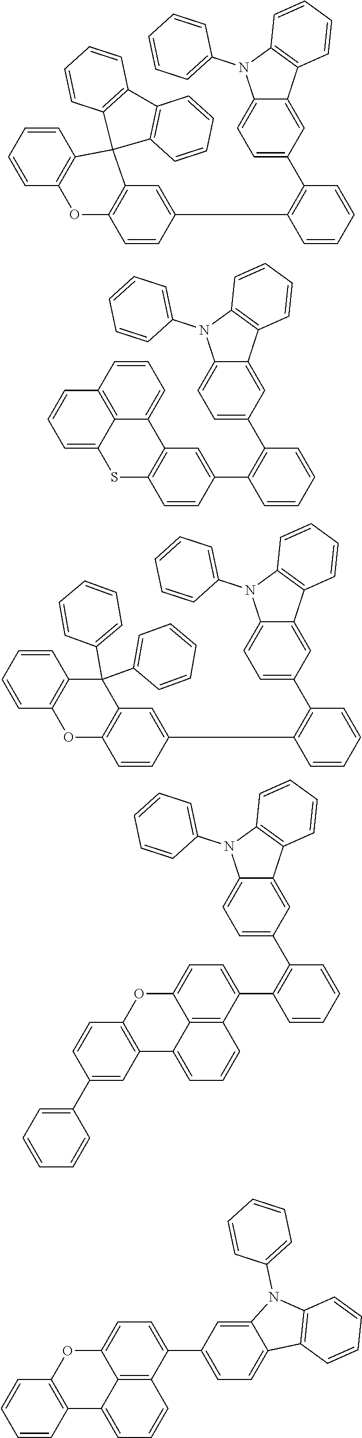

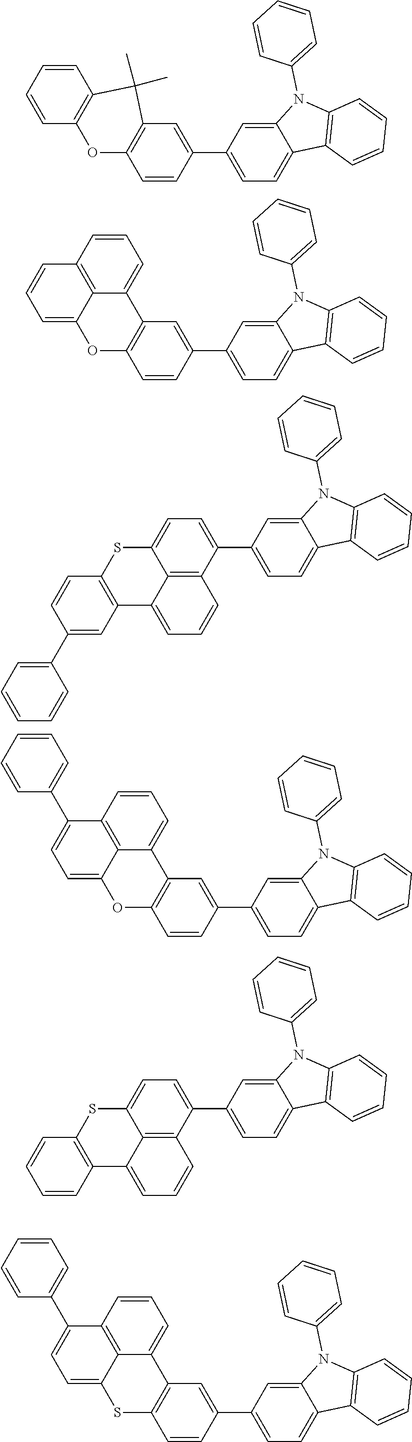

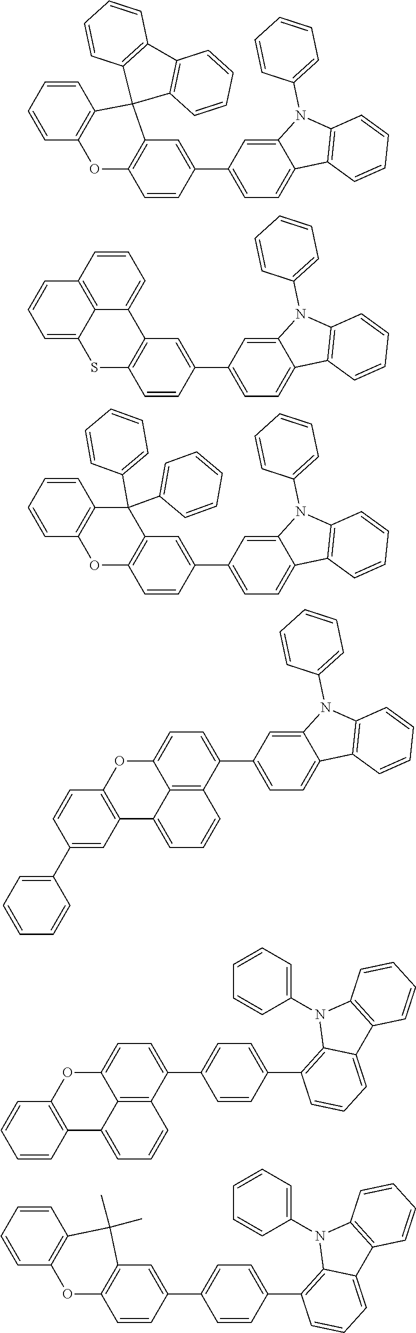

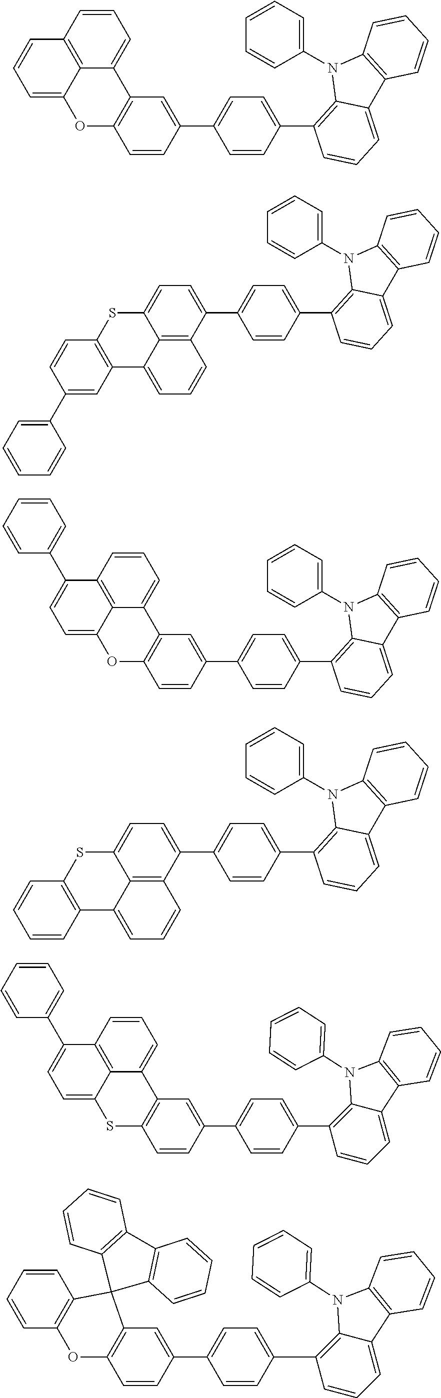

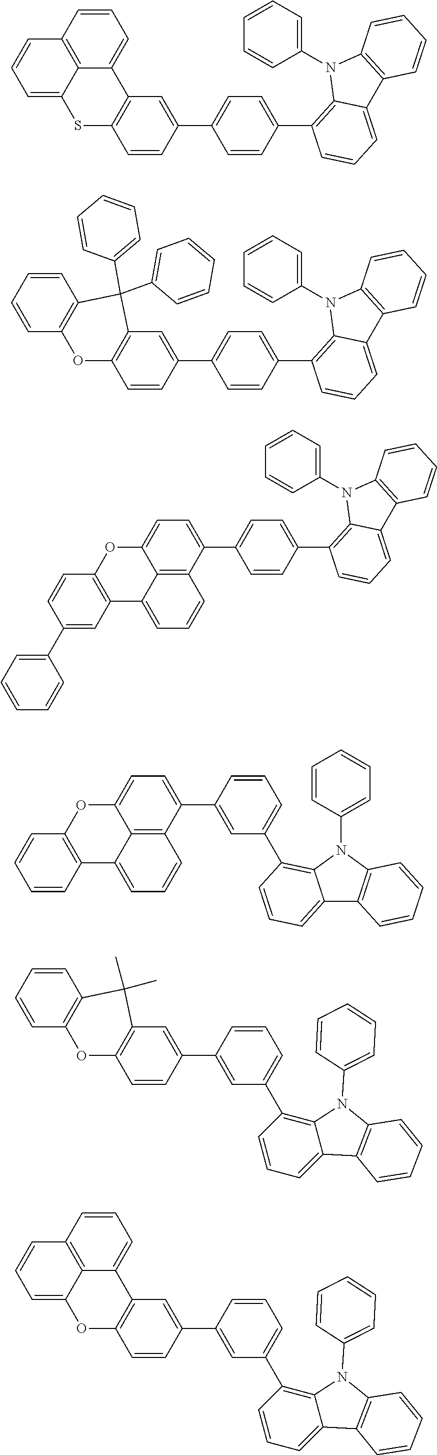 [SMILES: c1ccc(-n2c3ccccc3c3cccc(-c4ccc(-c5ccc6c(c5)-c5cccc7cccc(c57)S6)cc4)c32)cc1 ; c1ccc(-n2c3ccccc3c3cccc(-c4ccc(-c5ccc6c(c5)C(c5ccccc5)(c5ccccc5)c5ccccc5O6)cc4)c32)cc1 ; c1ccc(-c2ccc3c(c2)-c2cccc4c(-c5ccc(-c6cccc7c8ccccc8n(-c8ccccc8)c67)cc5)ccc(c24)O3)cc1 ; c1ccc(-n2c3ccccc3c3cccc(-c4cccc(-c5ccc6c7c(cccc57)-c5ccccc5O6)c4)c32)cc1 ; CC1(C)c2ccccc2Oc2ccc(-c3cccc(-c4cccc5c6ccccc6n(-c6ccccc6)c45)c3)cc21 ; c1ccc(-n2c3ccccc3c3cccc(-c4cccc(-c5ccc6c(c5)-c5cccc7cccc(c57)O6)c4)c32)cc1]

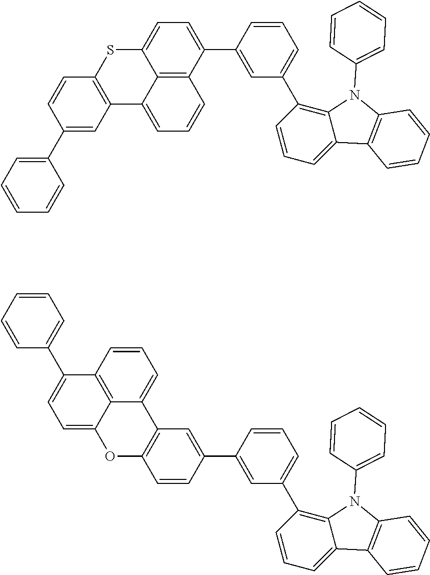 [SMILES: c1ccc(-c2ccc3c(c2)-c2cccc4c(-c5cccc(-c6cccc7c8ccccc8n(-c8ccccc8)c67)c5)ccc(c24)S3)cc1 ; c1ccc(-c2ccc3c4c(cccc24)-c2cc(-c4cccc(-c5cccc6c7ccccc7n(-c7ccccc7)c56)c4)ccc2O3)cc1]

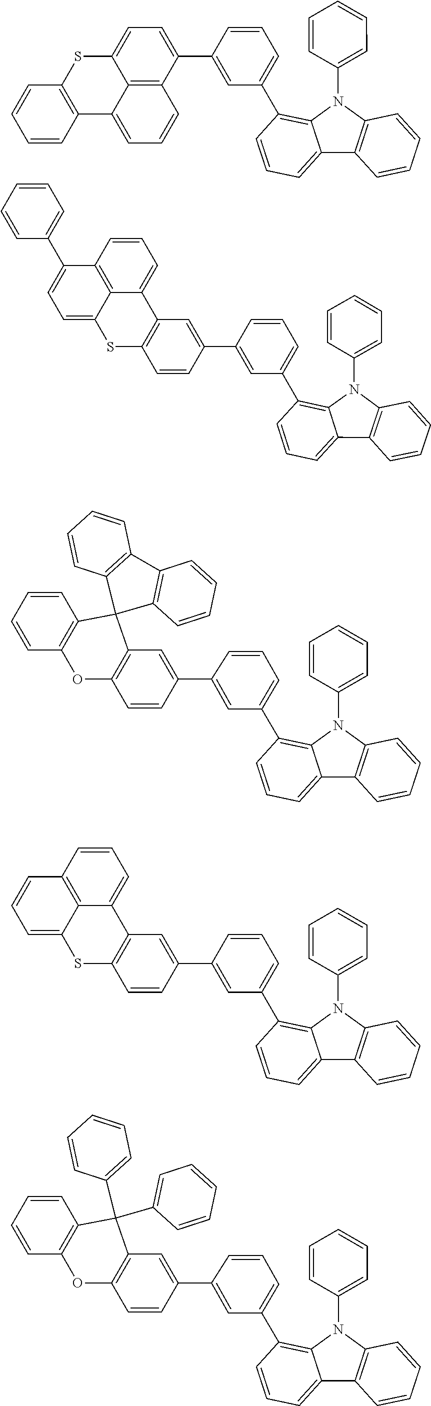

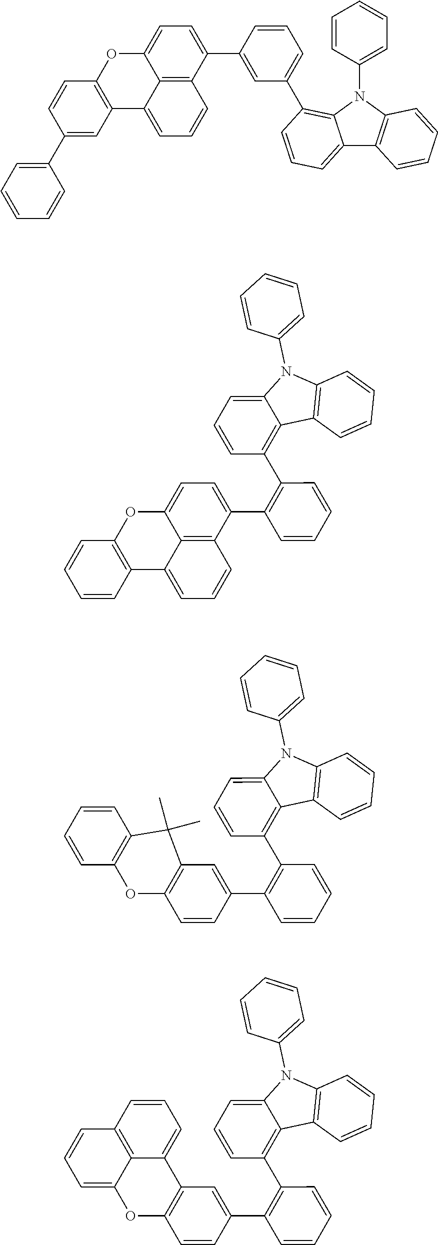 [SMILES: c1ccc(-c2ccc3c(c2)-c2cccc4c(-c5cccc(-c6cccc7c8ccccc8n(-c8ccccc8)c67)c5)ccc(c24)O3)cc1 ; c1ccc(-n2c3ccccc3c3c(-c4ccccc4-c4ccc5c6c(cccc46)-c4ccccc4O5)cccc32)cc1 ; CC1(C)c2ccccc2Oc2ccc(-c3ccccc3-c3cccc4c3c3ccccc3n4-c3ccccc3)cc21 ; c1ccc(-n2c3ccccc3c3c(-c4ccccc4-c4ccc5c(c4)-c4cccc6cccc(c46)O5)cccc32)cc1]

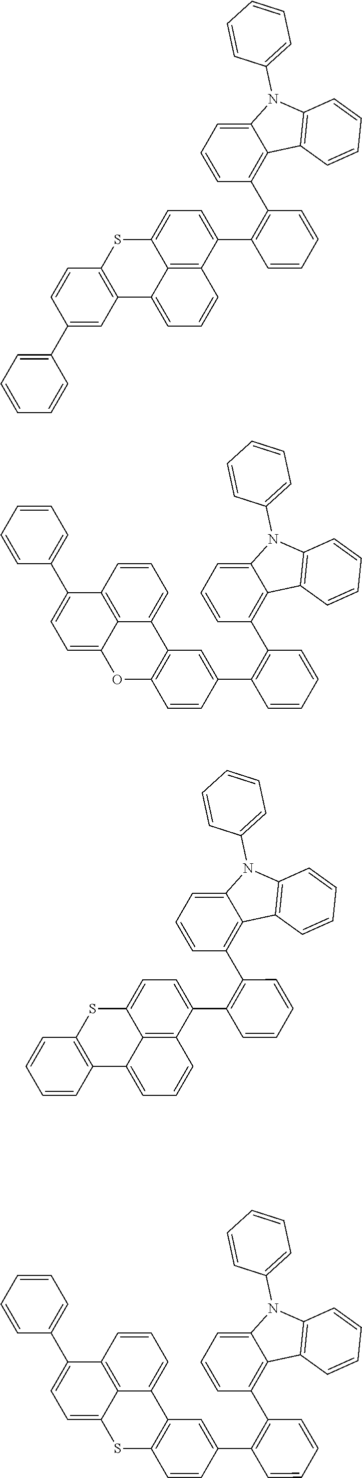 [SMILES: c1ccc(-c2ccc3c(c2)-c2cccc4c(-c5ccccc5-c5cccc6c5c5ccccc5n6-c5ccccc5)ccc(c24)S3)cc1 ; c1ccc(-c2ccc3c4c(cccc24)-c2cc(-c4ccccc4-c4cccc5c4c4ccccc4n5-c4ccccc4)ccc2O3)cc1 ; c1ccc(-n2c3ccccc3c3c(-c4ccccc4-c4ccc5c6c(cccc46)-c4ccccc4S5)cccc32)cc1 ; c1ccc(-c2ccc3c4c(cccc24)-c2cc(-c4ccccc4-c4cccc5c4c4ccccc4n5-c4ccccc4)ccc2S3)cc1]

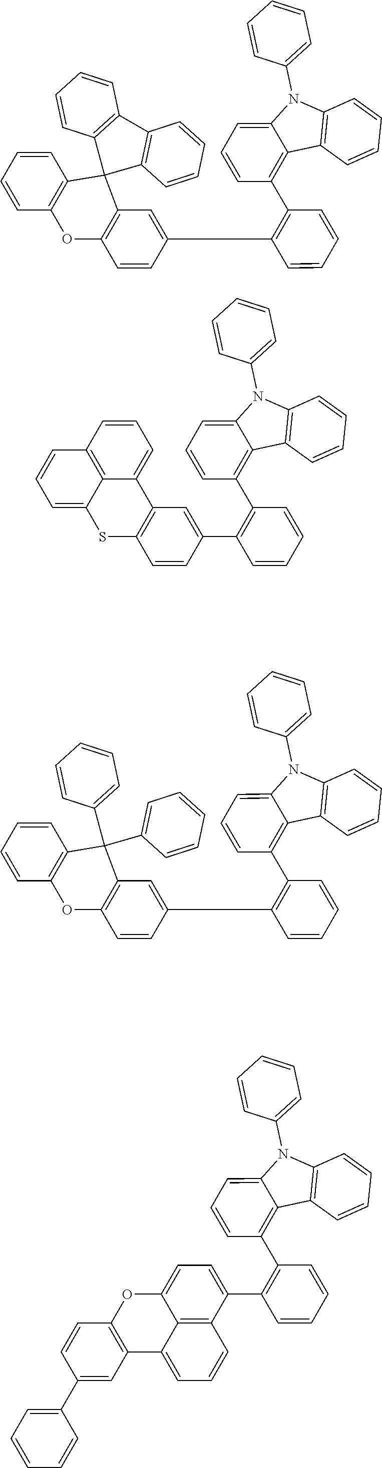

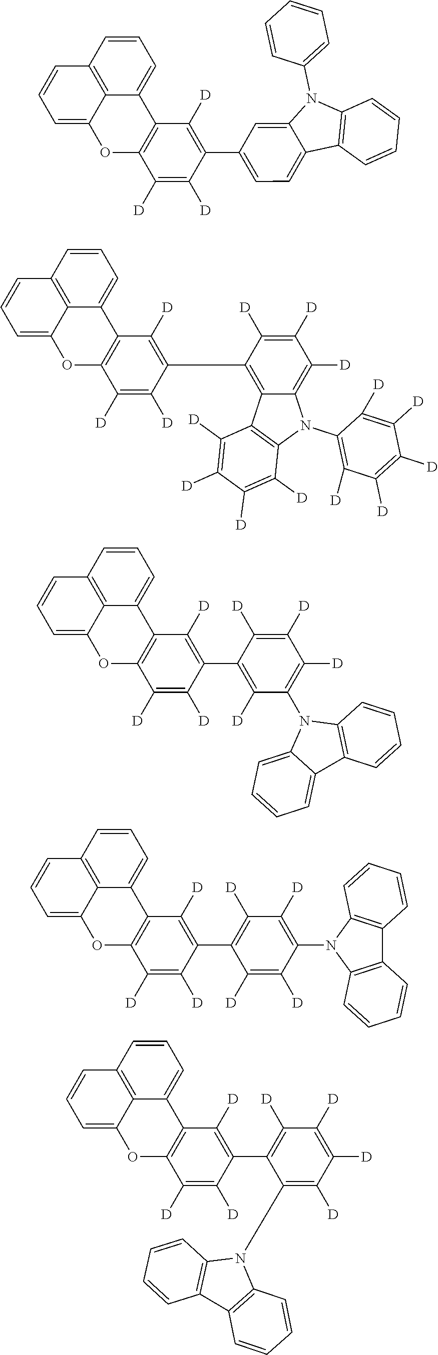

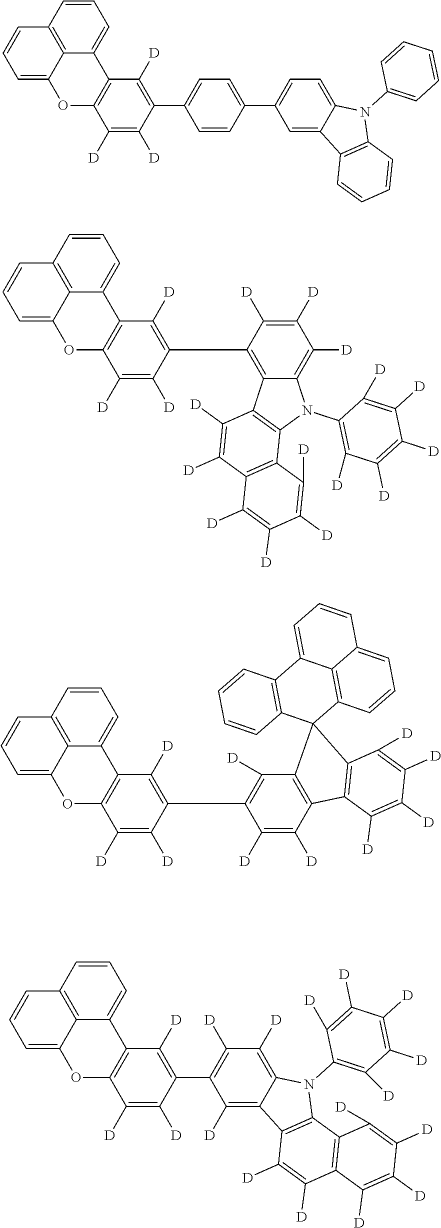

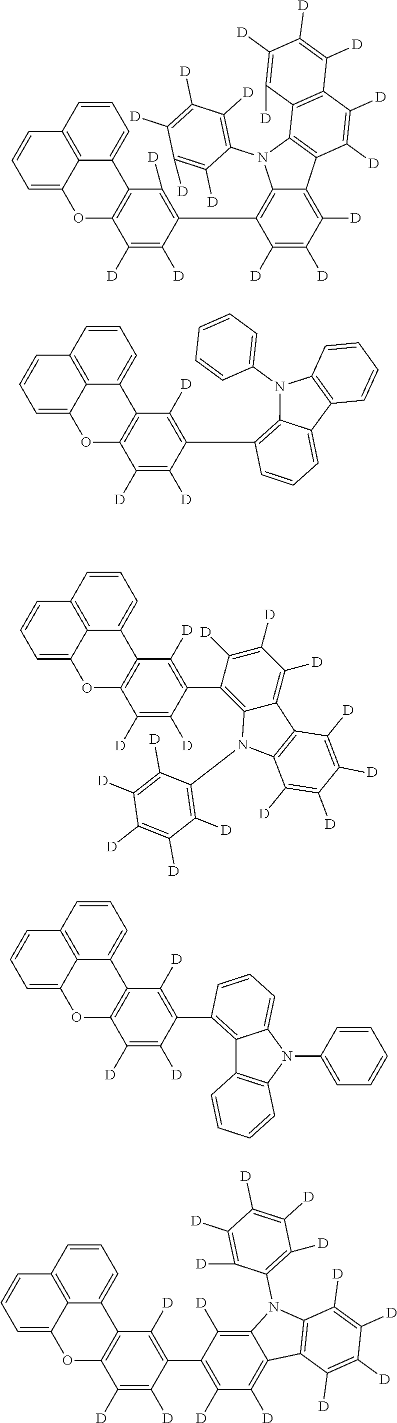

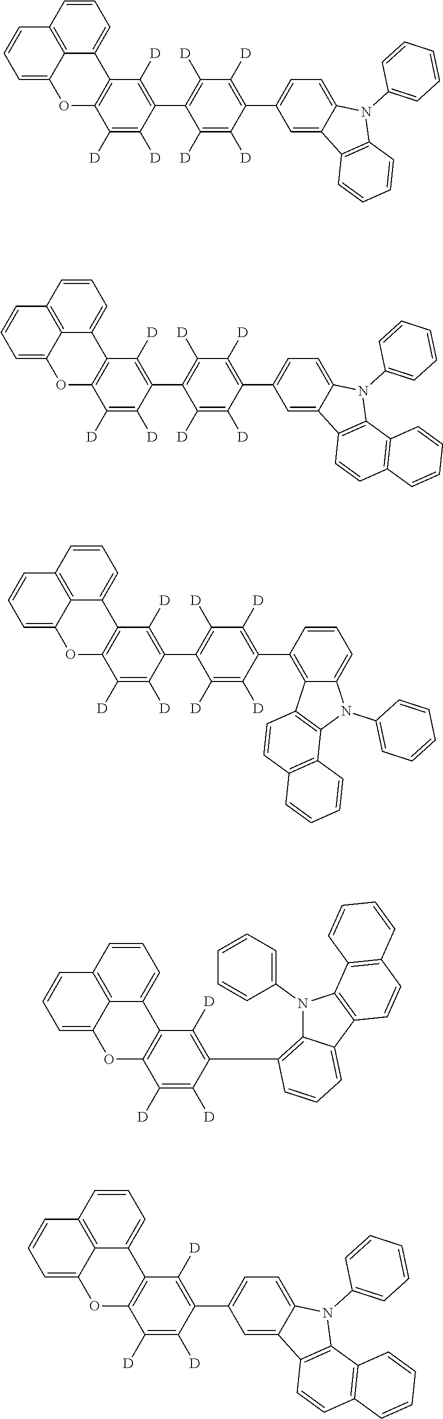

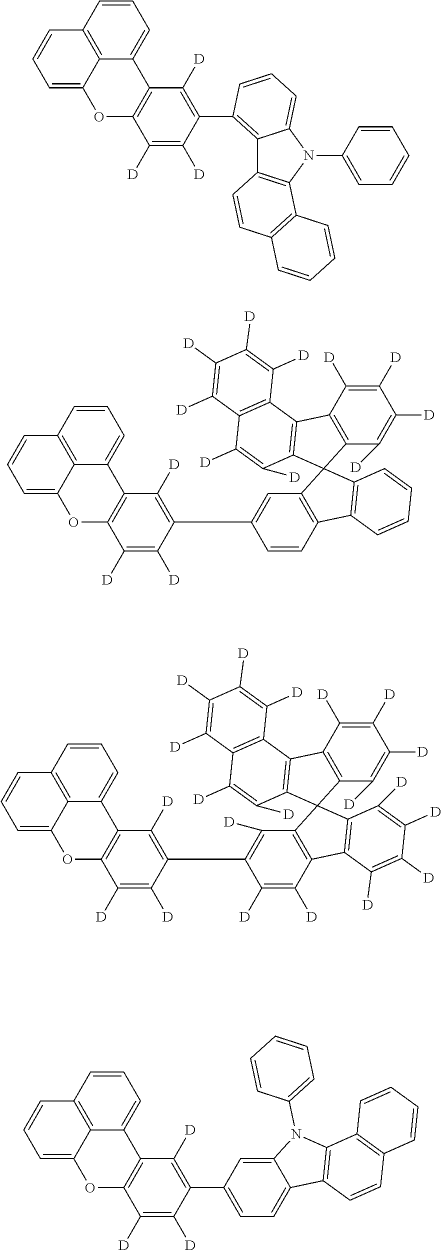

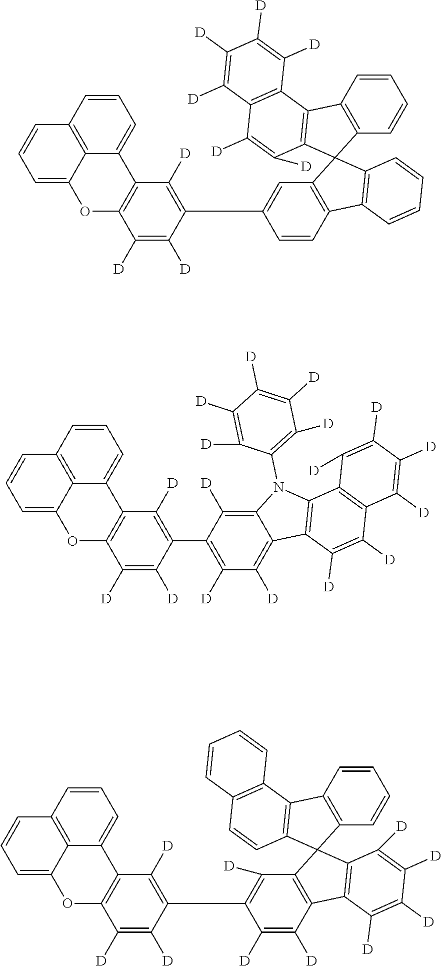 [SMILES: [2H]c1c([2H])c(-c2ccc3c(c2)C2(c4ccccc4-3)c3ccccc3-c3c2c([2H])c([2H])c2c([2H])c([2H])c([2H])c([2H])c32)c([2H])c2c1Oc1cccc3cccc-2c13 ; [2H]c1c([2H])c([2H])c(-n2c3c([2H])c(-c4c([2H])c([2H])c5c(c4[2H])-c4cccc6cccc(c46)O5)c([2H])c([2H])c3c3c([2H])c([2H])c4c([2H])c([2H])c([2H])c([2H])c4c32)c([2H])c1[2H] ; [2H]c1c([2H])c([2H])c2c(c1[2H])-c1c([2H])c([2H])c(-c3c([2H])c([2H])c4c(c3[2H])-c3cccc5cccc(c35)O4)c([2H])c1C21c2ccccc2-c2c1ccc1ccccc21]

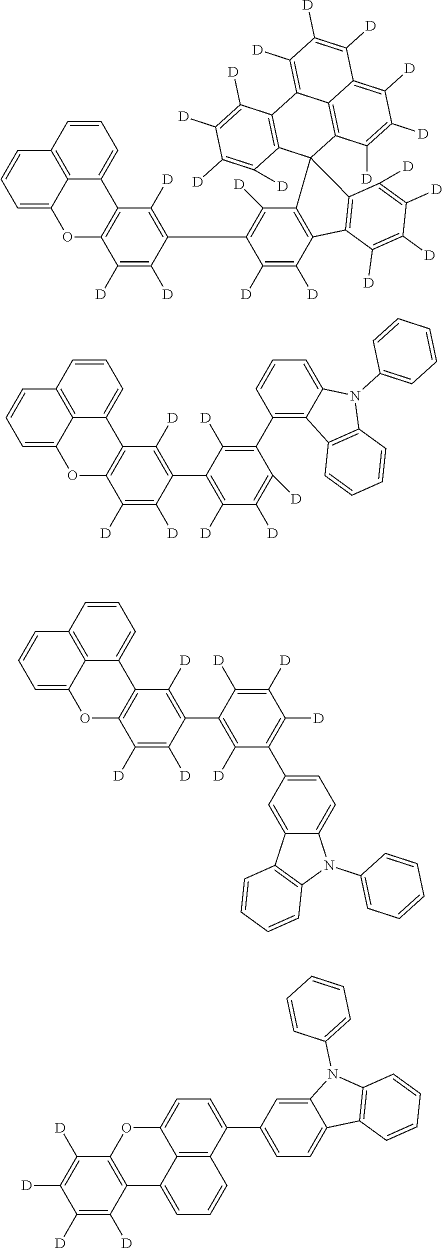

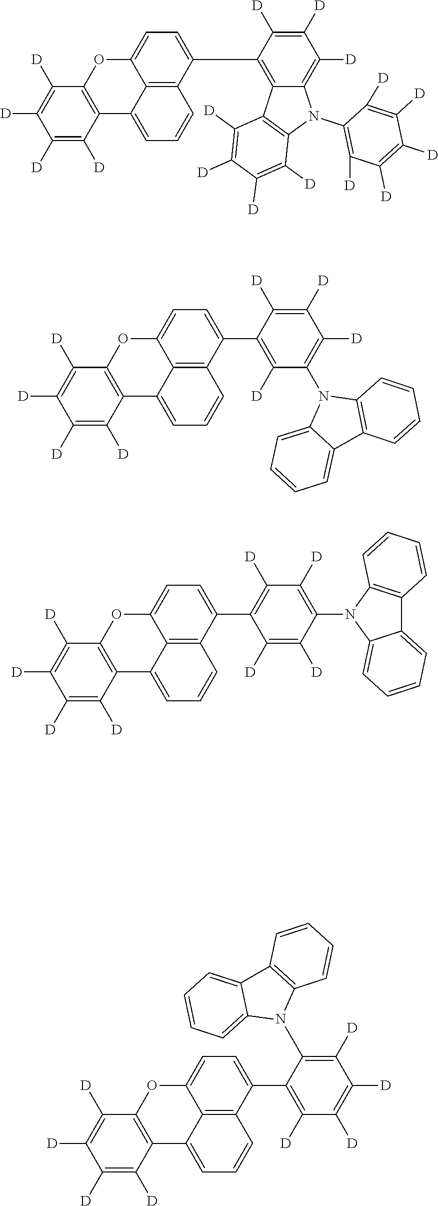 [SMILES: [2H]c1c([2H])c([2H])c(-n2c3c([2H])c([2H])c([2H])c([2H])c3c3c(-c4ccc5c6c(cccc46)-c4c([2H])c([2H])c([2H])c([2H])c4O5)c([2H])c([2H])c([2H])c32)c([2H])c1[2H] ; [2H]c1c([2H])c([2H])c2c(c1[2H])Oc1ccc(-c3c([2H])c([2H])c([2H])c(-n4c5ccccc5c5ccccc54)c3[2H])c3cccc-2c13 ; [2H]c1c([2H])c([2H])c2c(c1[2H])Oc1ccc(-c3c([2H])c([2H])c(-n4c5ccccc5c5ccccc54)c([2H])c3[2H])c3cccc-2c13 ; [2H]c1c([2H])c([2H])c2c(c1[2H])Oc1ccc(-c3c([2H])c([2H])c([2H])c([2H])c3-n3c4ccccc4c4ccccc43)c3cccc-2c13]

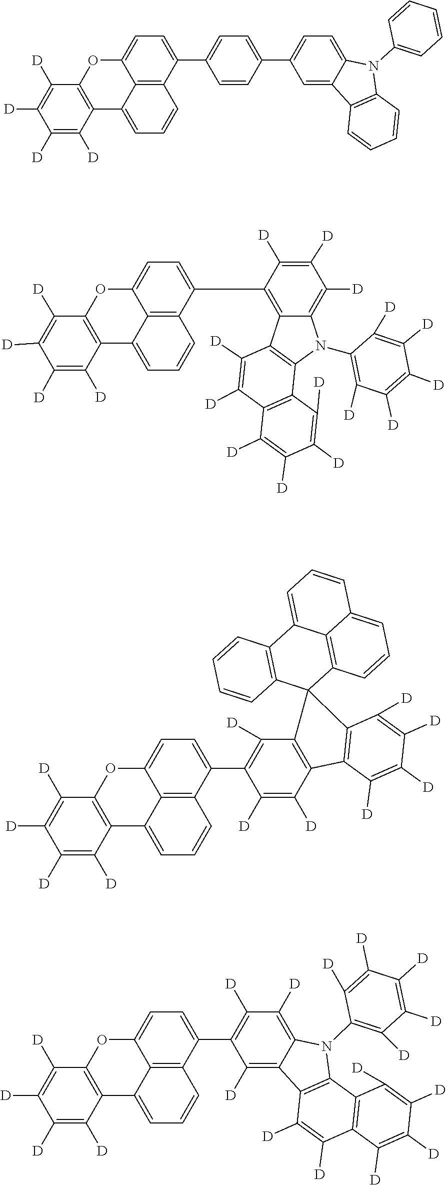

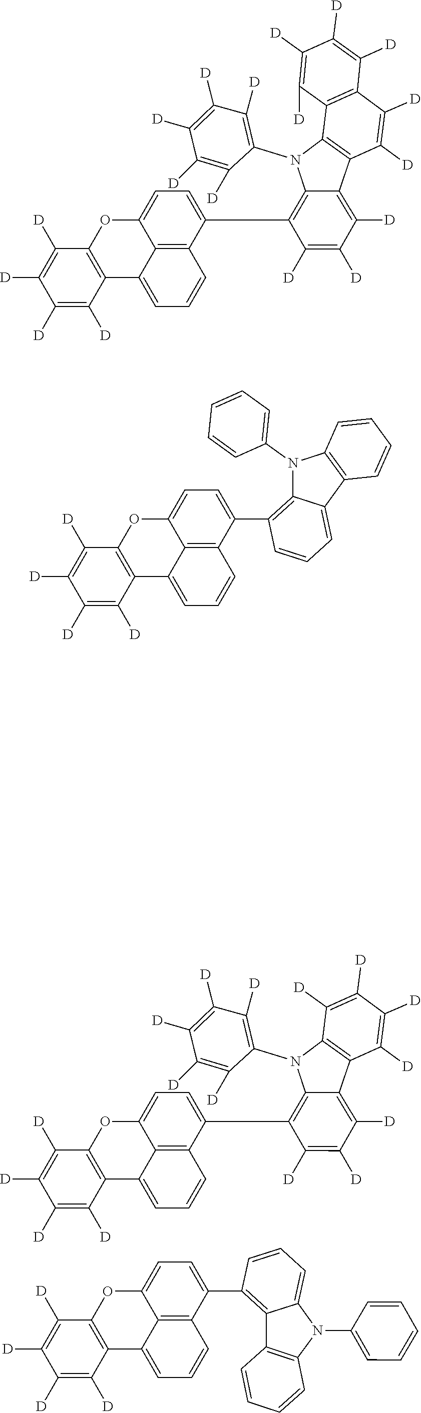 [SMILES: [2H]c1c([2H])c([2H])c(-n2c3c(-c4ccc5c6c(cccc46)-c4c([2H])c([2H])c([2H])c([2H])c4O5)c([2H])c([2H])c([2H])c3c3c([2H])c([2H])c4c([2H])c([2H])c([2H])c([2H])c4c32)c([2H])c1[2H] ; [2H]c1c([2H])c([2H])c2c(c1[2H])Oc1ccc(-c3cccc4c5ccccc5n(-c5ccccc5)c34)c3cccc-2c13 ; [2H]c1c([2H])c([2H])c(-n2c3c([2H])c([2H])c([2H])c([2H])c3c3c([2H])c([2H])c([2H])c(-c4ccc5c6c(cccc46)-c4c([2H])c([2H])c([2H])c([2H])c4O5)c32)c([2H])c1[2H] ; [2H]c1c([2H])c([2H])c2c(c1[2H])Oc1ccc(-c3cccc4c3c3ccccc3n4-c3ccccc3)c3cccc-2c13]

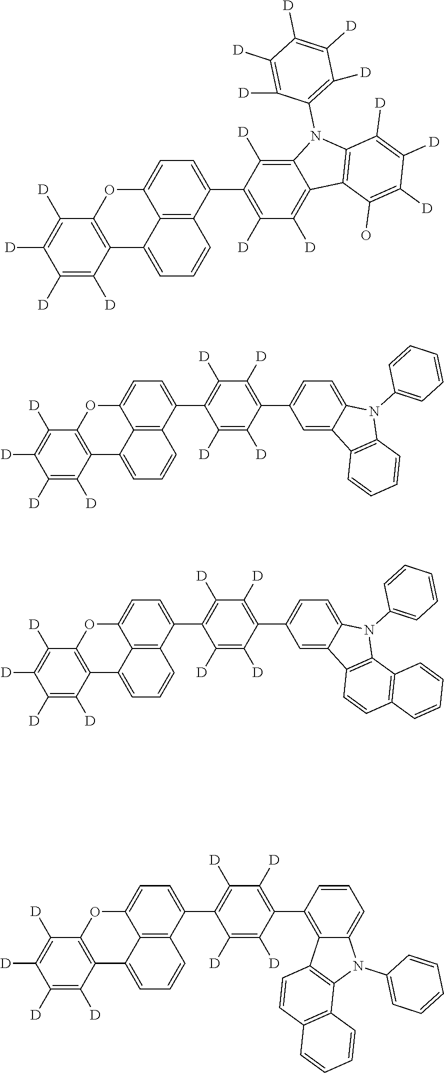

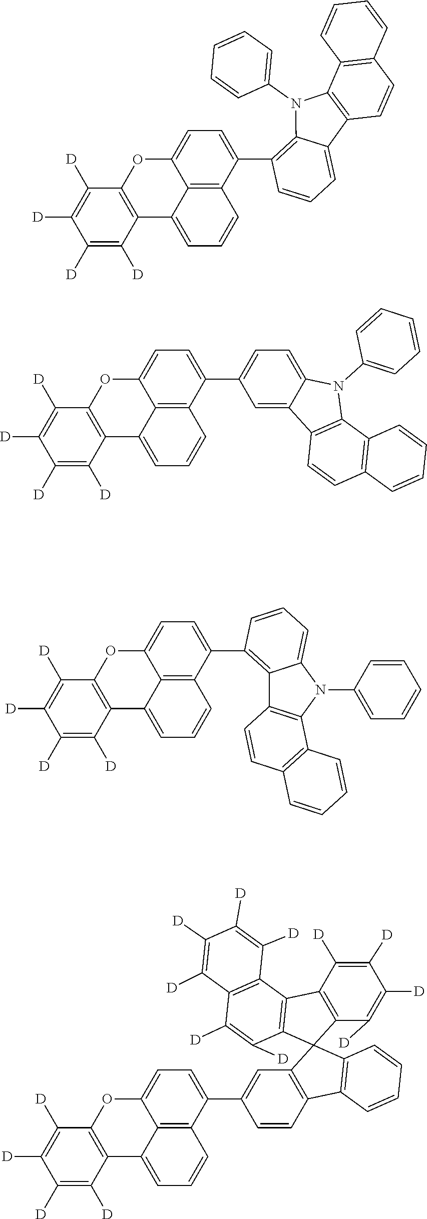 [SMILES: [2H]c1c([2H])c([2H])c2c(c1[2H])Oc1ccc(-c3cccc4c5ccc6ccccc6c5n(-c5ccccc5)c34)c3cccc-2c13 ; [2H]c1c([2H])c([2H])c2c(c1[2H])Oc1ccc(-c3ccc4c(c3)c3ccc5ccccc5c3n4-c3ccccc3)c3cccc-2c13 ; [2H]c1c([2H])c([2H])c2c(c1[2H])Oc1ccc(-c3cccc4c3c3ccc5ccccc5c3n4-c3ccccc3)c3cccc-2c13 ; [2H]c1c([2H])c([2H])c2c(c1[2H])Oc1ccc(-c3ccc4c(c3)C3(c5ccccc5-4)c4c([2H])c([2H])c([2H])c([2H])c4-c4c3c([2H])c([2H])c3c([2H])c([2H])c([2H])c([2H])c43)c3cccc-2c13]

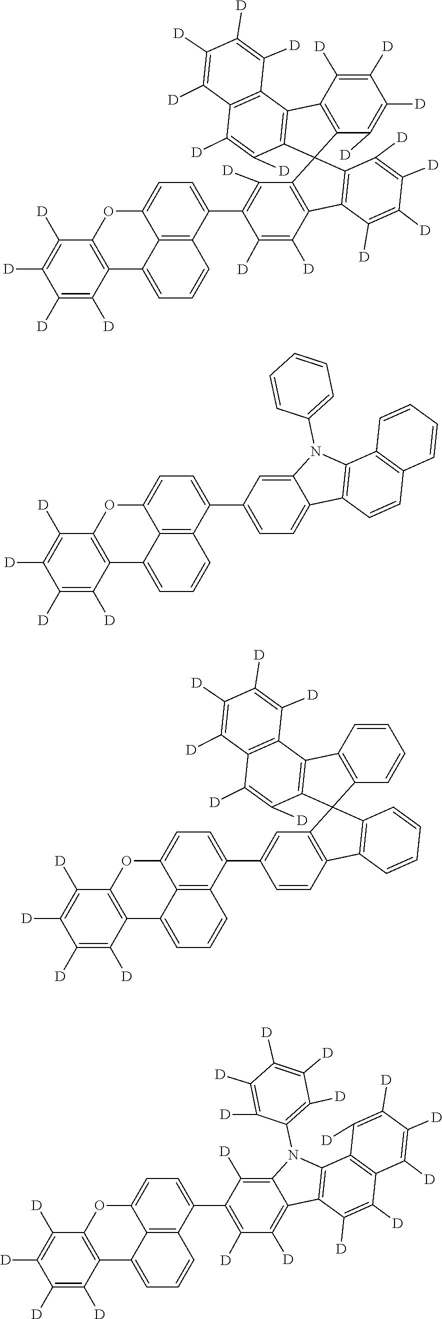

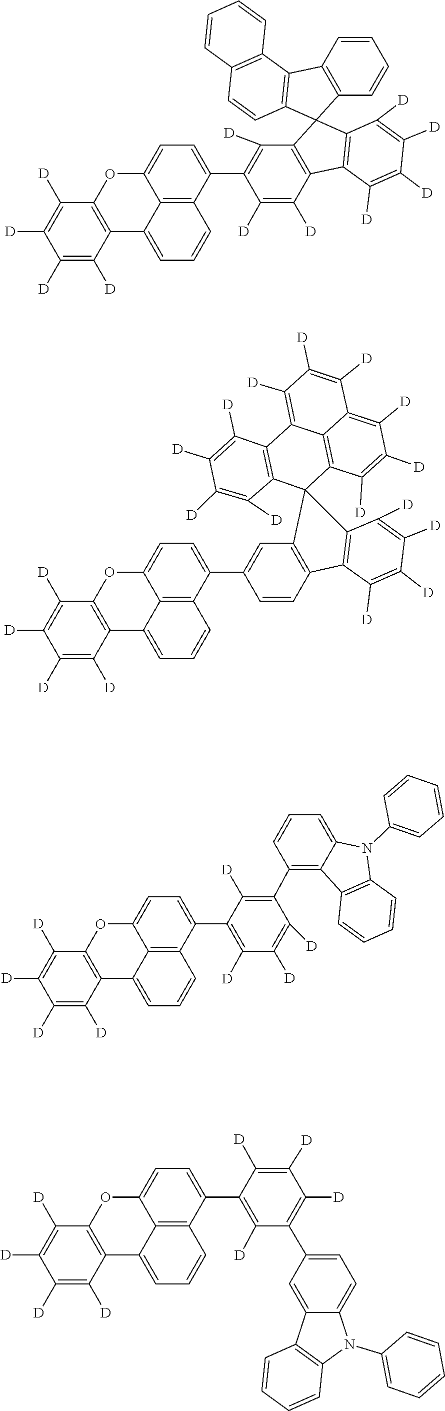

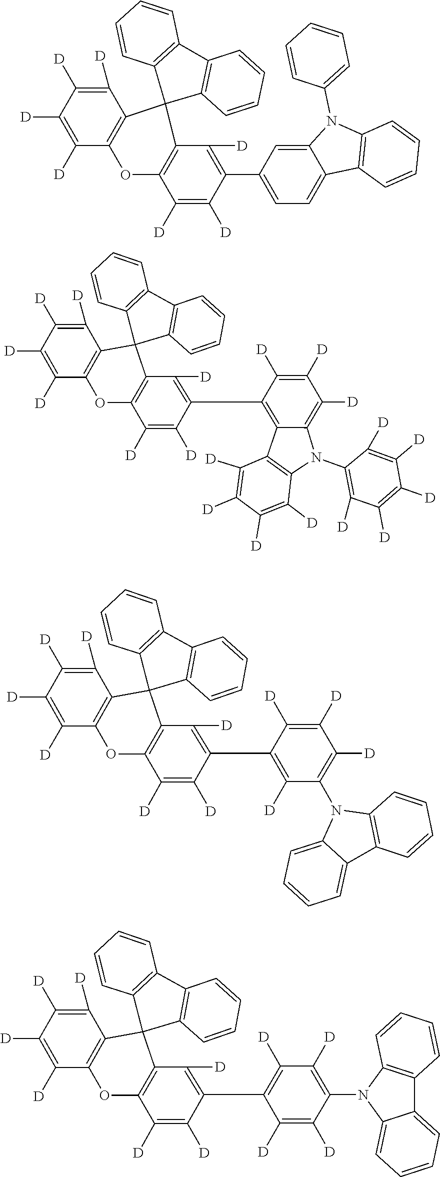

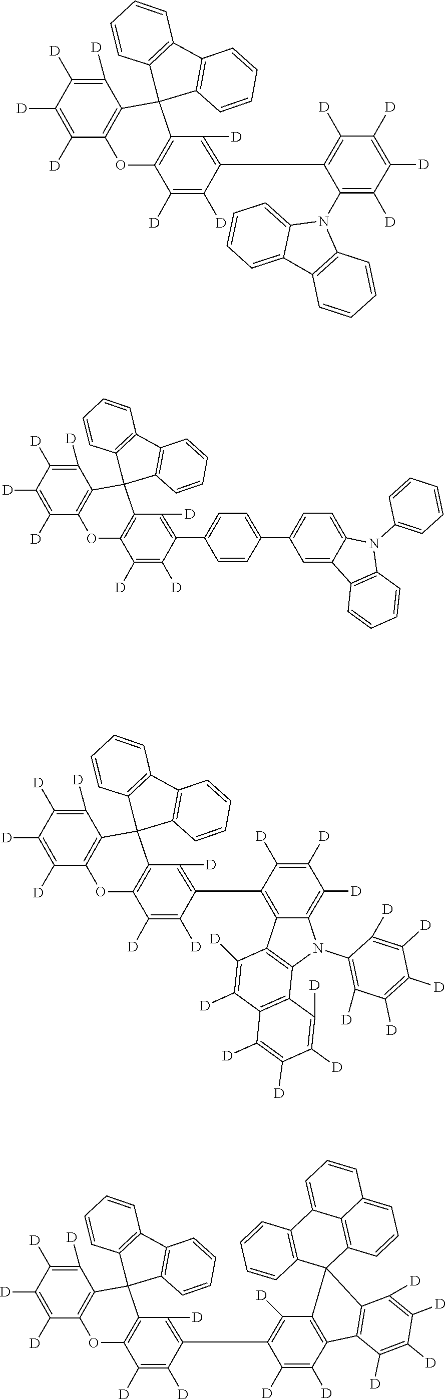

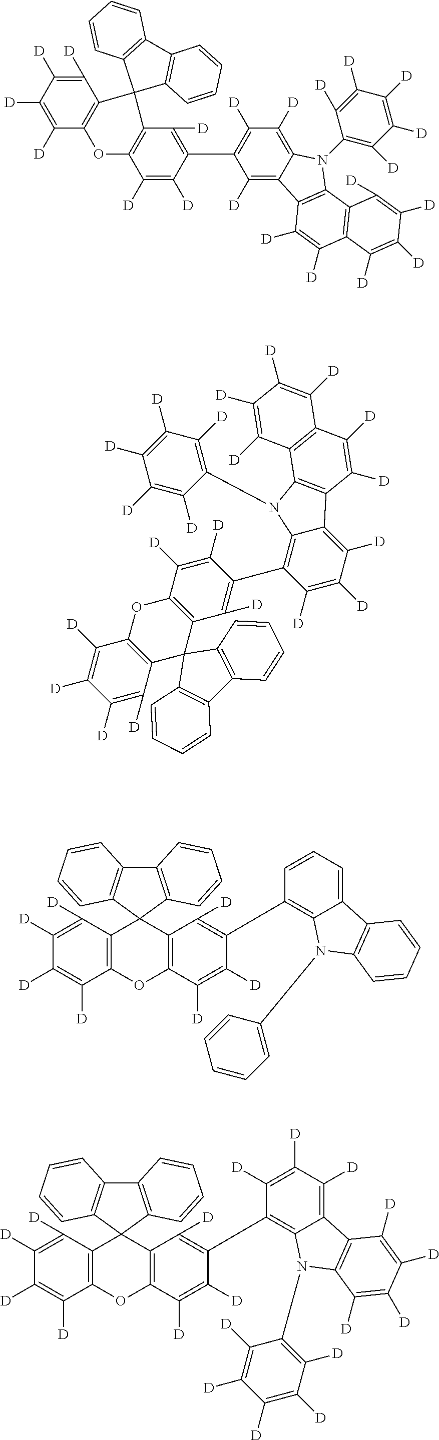

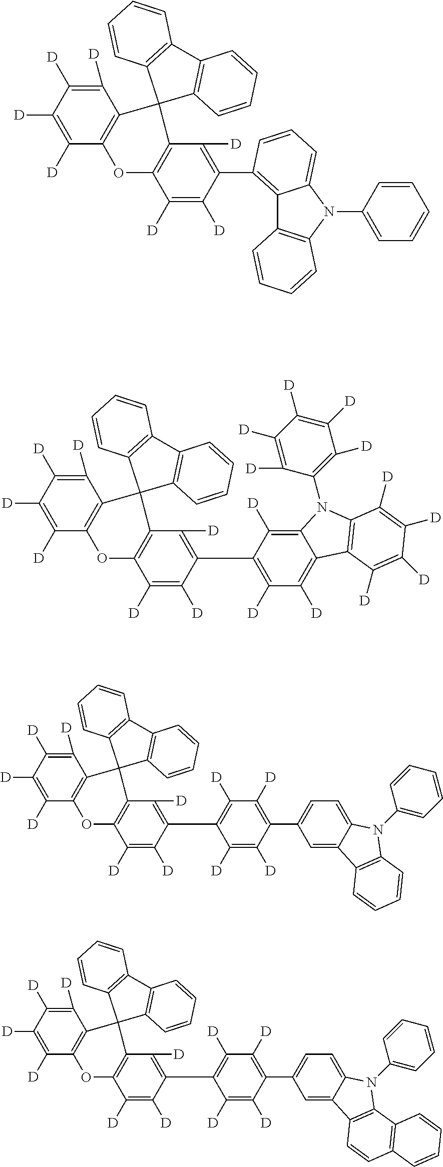

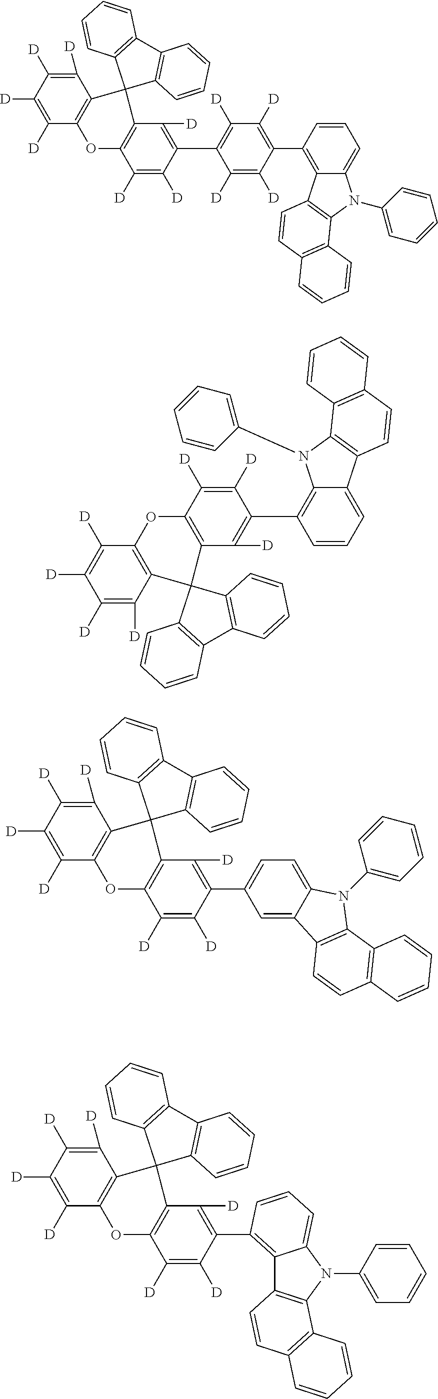

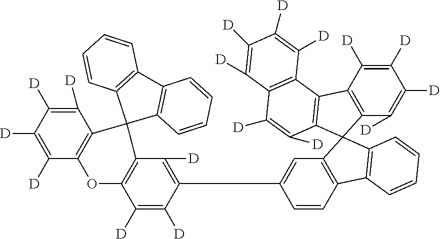 [SMILES: [2H]c1c([2H])c([2H])c2c(c1[2H])Oc1c([2H])c([2H])c(-c3ccc4c(c3)C3(c5ccccc5-4)c4c([2H])c([2H])c([2H])c([2H])c4-c4c3c([2H])c([2H])c3c([2H])c([2H])c([2H])c([2H])c43)c([2H])c1C21c2ccccc2-c2ccccc21]

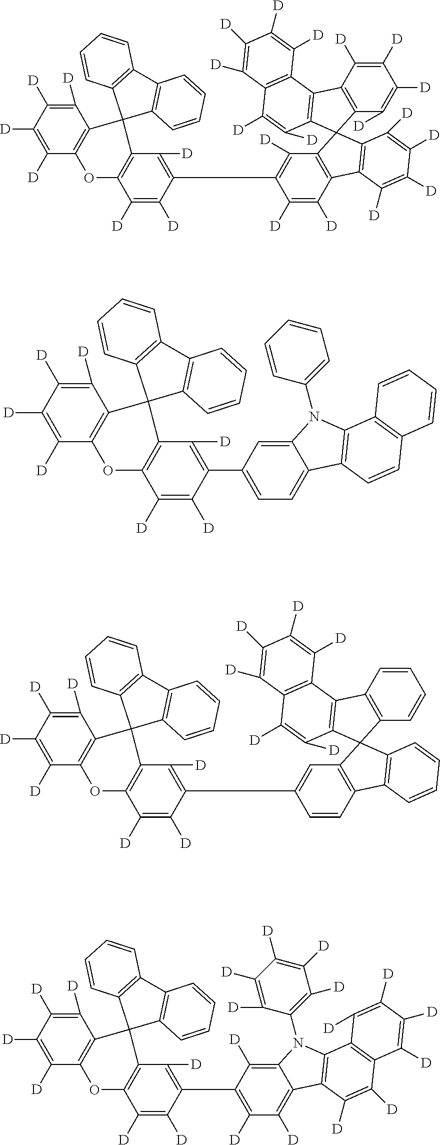

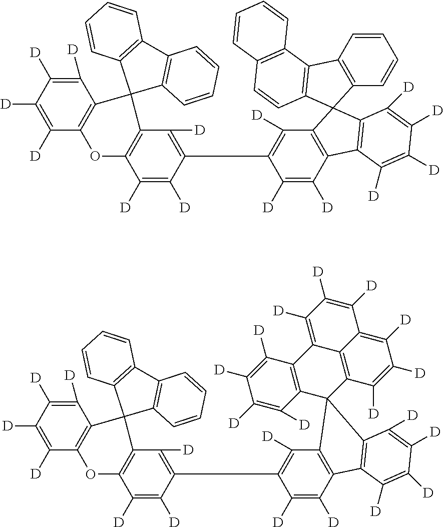 [SMILES: [2H]c1c([2H])c([2H])c2c(c1[2H])Oc1c([2H])c([2H])c(-c3c([2H])c([2H])c4c(c3[2H])C3(c5ccccc5-c5c3ccc3ccccc53)c3c([2H])c([2H])c([2H])c([2H])c3-4)c([2H])c1C21c2ccccc2-c2ccccc21 ; [2H]c1c([2H])c([2H])c2c(c1[2H])Oc1c([2H])c([2H])c(-c3c([2H])c([2H])c4c(c3[2H])C3(c5c([2H])c([2H])c([2H])c([2H])c5-4)c4c([2H])c([2H])c([2H])c([2H])c4-c4c([2H])c([2H])c([2H])c5c([2H])c([2H])c([2H])c3c45)c([2H])c1C21c2ccccc2-c2ccccc21]

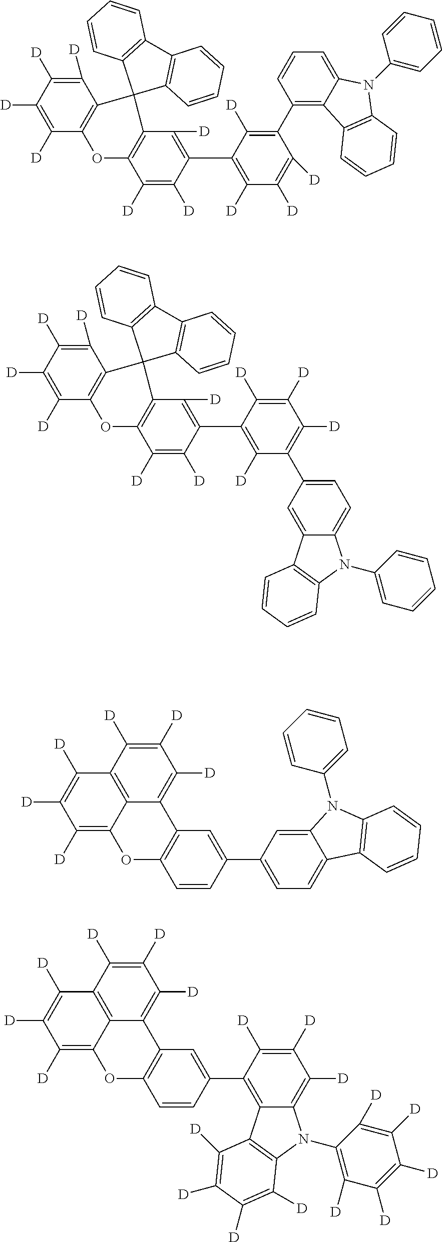

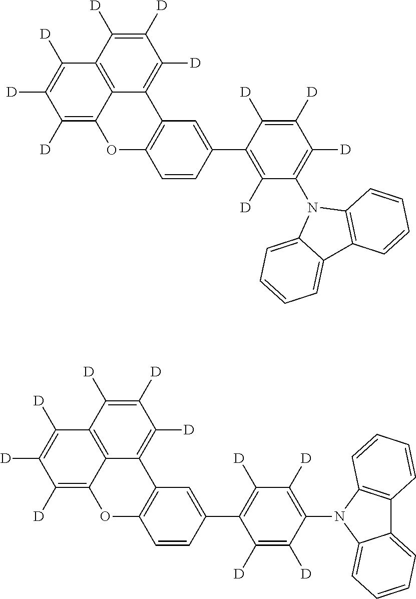 [SMILES: [2H]c1c([2H])c(-c2ccc3c(c2)-c2c([2H])c([2H])c([2H])c4c([2H])c([2H])c([2H])c(c24)O3)c([2H])c(-n2c3ccccc3c3ccccc32)c1[2H] ; [2H]c1c([2H])c(-n2c3ccccc3c3ccccc32)c([2H])c([2H])c1-c1ccc2c(c1)-c1c([2H])c([2H])c([2H])c3c([2H])c([2H])c([2H])c(c13)O2]

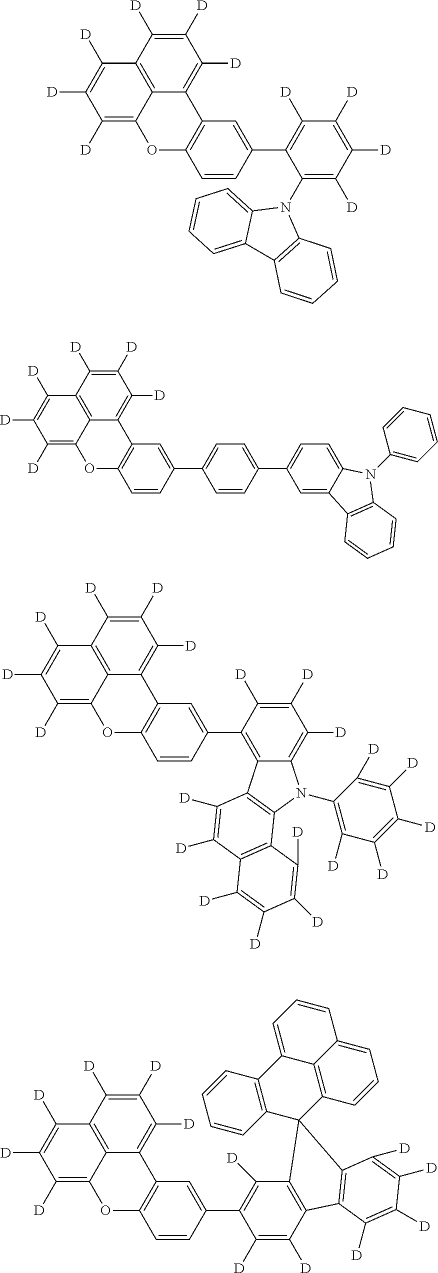

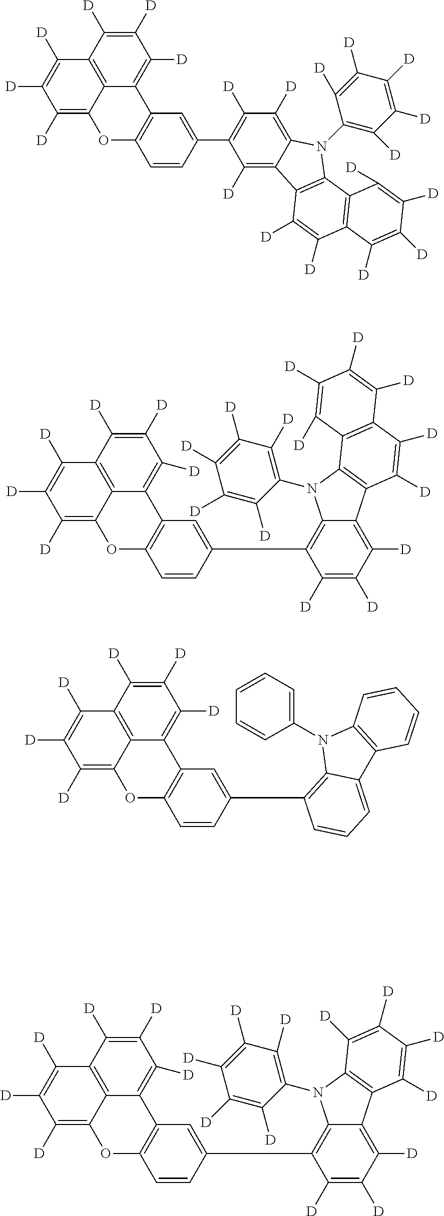

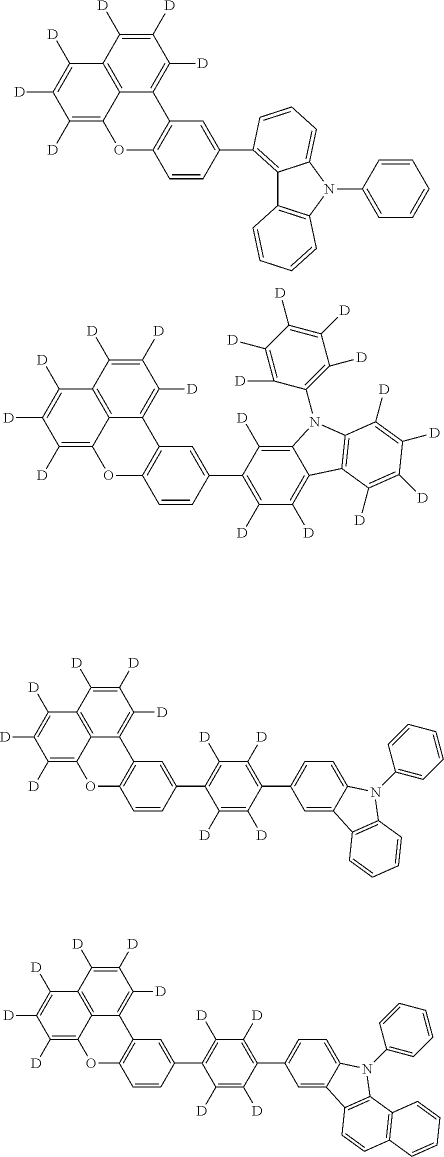

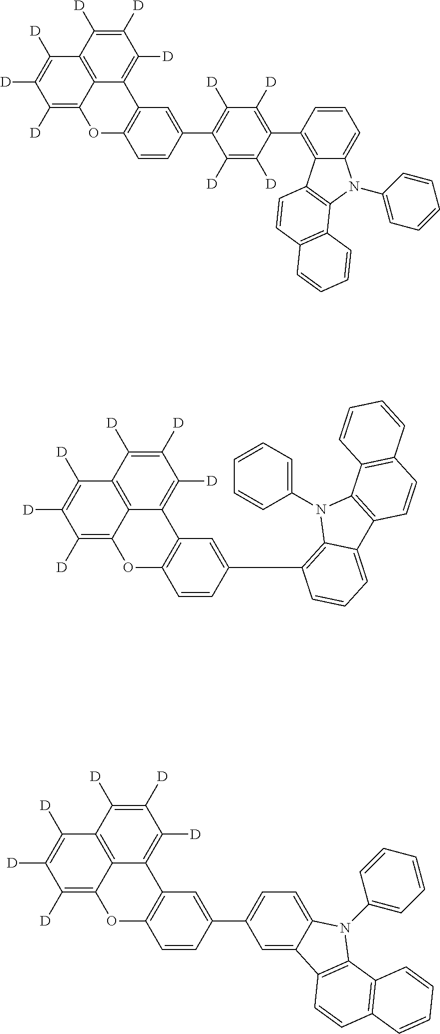 [SMILES: [2H]c1c([2H])c(-c2cccc3c2c2ccc4ccccc4c2n3-c2ccccc2)c([2H])c([2H])c1-c1ccc2c(c1)-c1c([2H])c([2H])c([2H])c3c([2H])c([2H])c([2H])c(c13)O2 ; [2H]c1c([2H])c2c3c(c([2H])c([2H])c([2H])c3c1[2H])-c1cc(-c3cccc4c5ccc6ccccc6c5n(-c5ccccc5)c34)ccc1O2 ; [2H]c1c([2H])c2c3c(c([2H])c([2H])c([2H])c3c1[2H])-c1cc(-c3ccc4c(c3)c3ccc5ccccc5c3n4-c3ccccc3)ccc1O2]

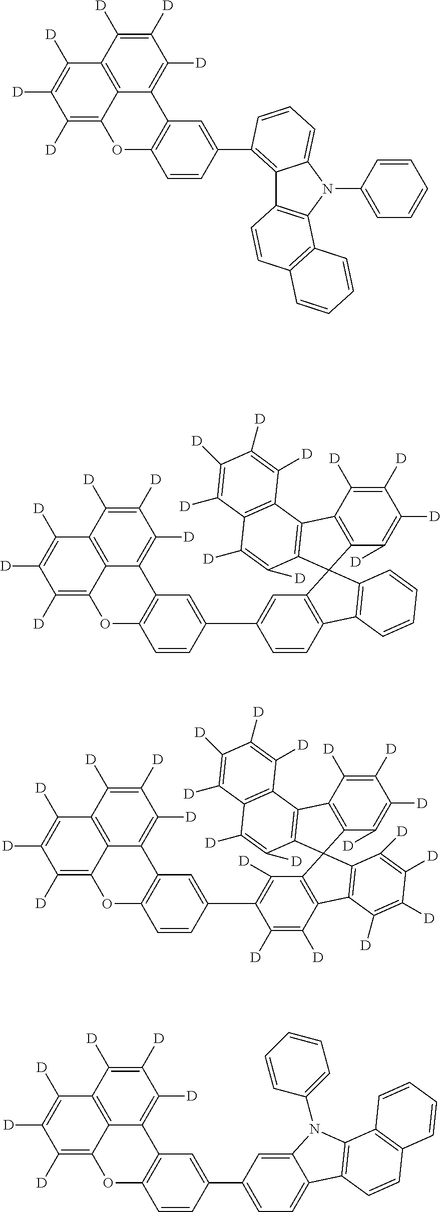

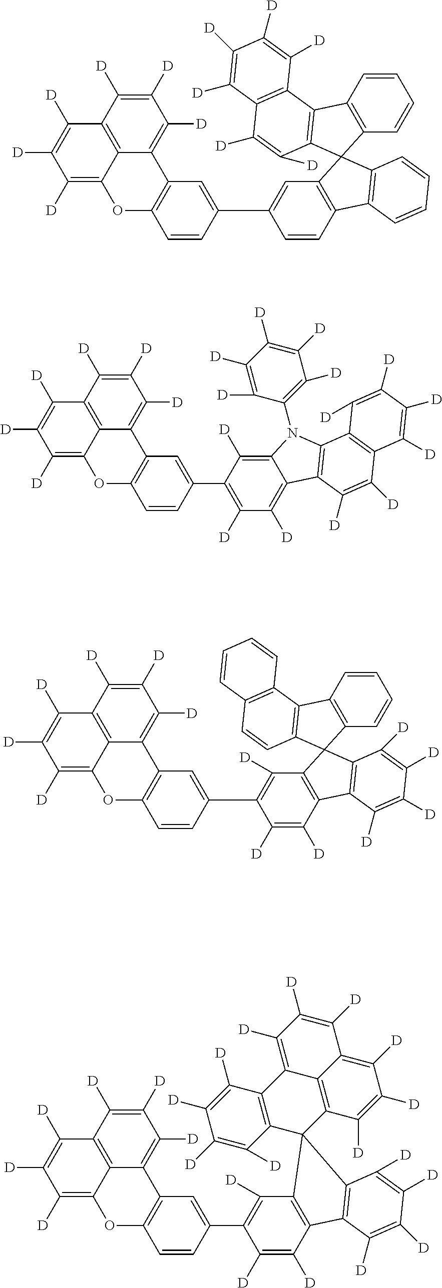

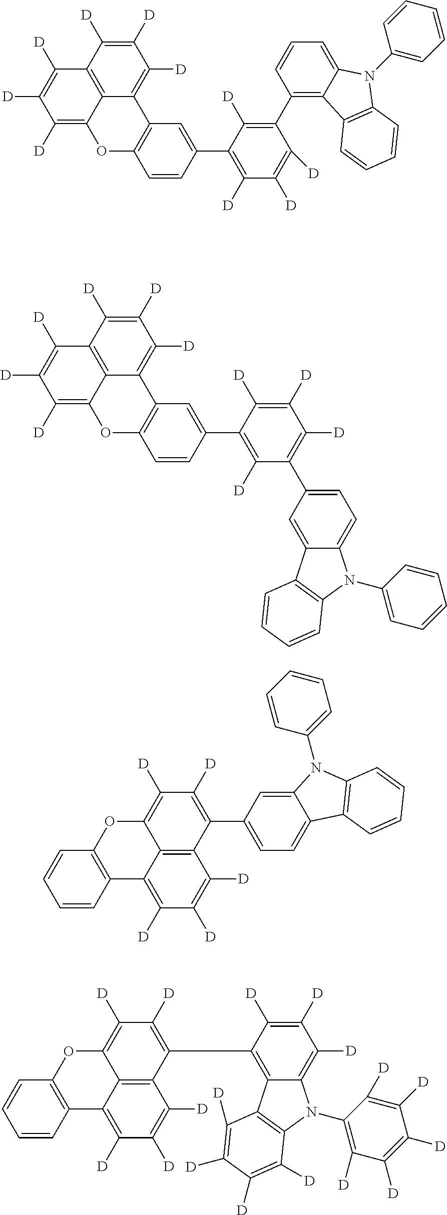

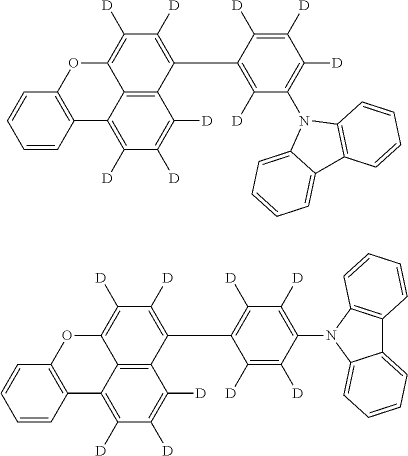 [SMILES: [2H]c1c([2H])c(-c2c([2H])c([2H])c3c4c(c([2H])c([2H])c([2H])c24)-c2ccccc2O3)c([2H])c(-n2c3ccccc3c3ccccc32)c1[2H] ; [2H]c1c([2H])c(-n2c3ccccc3c3ccccc32)c([2H])c([2H])c1-c1c([2H])c([2H])c2c3c(c([2H])c([2H])c([2H])c13)-c1ccccc1O2]

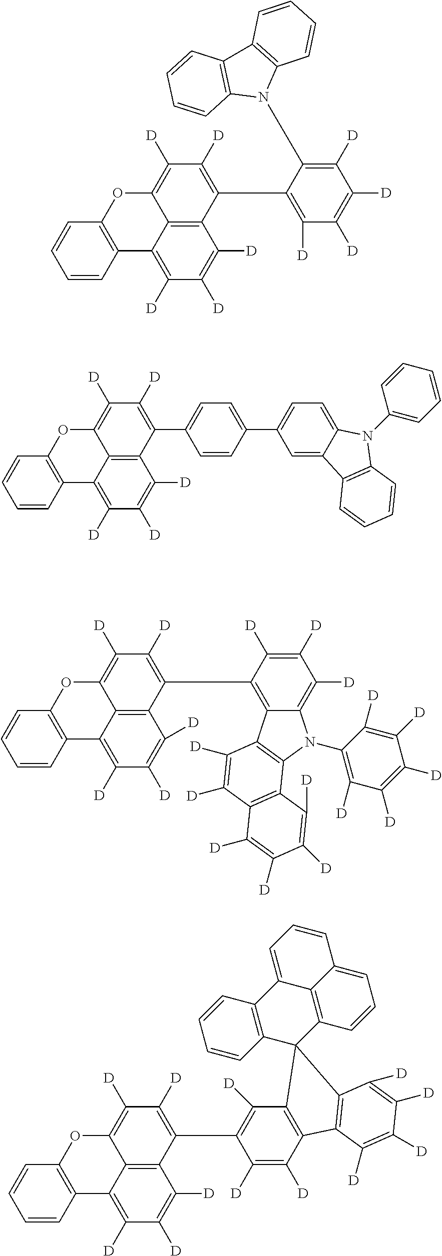

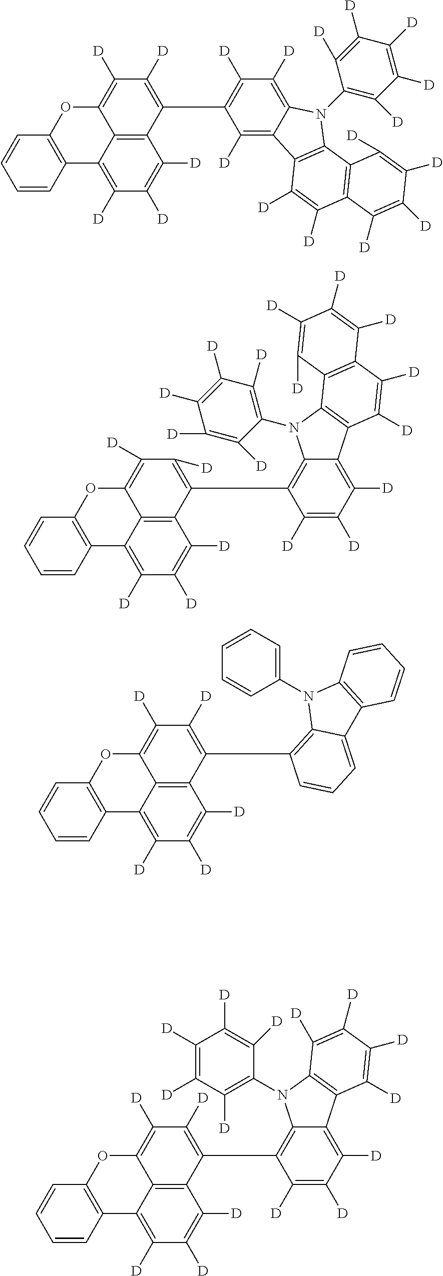

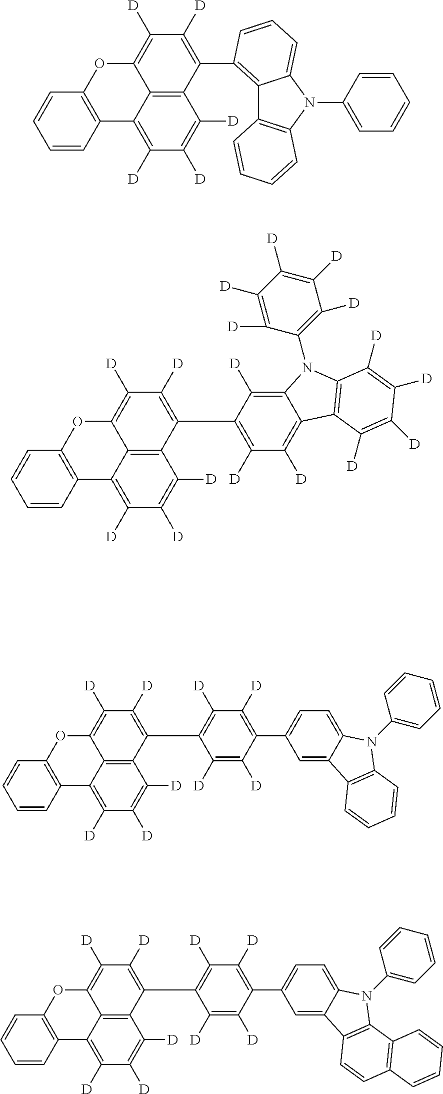

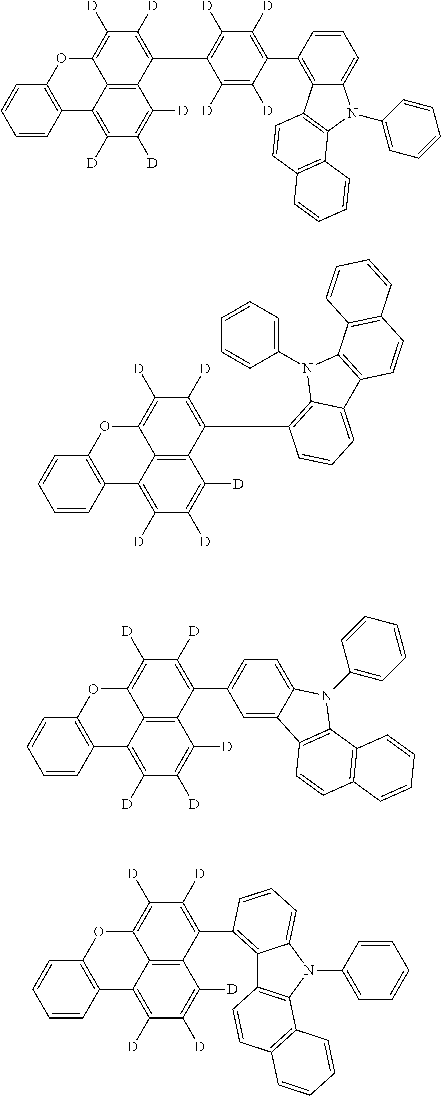 [SMILES: [2H]c1c([2H])c(-c2cccc3c2c2ccc4ccccc4c2n3-c2ccccc2)c([2H])c([2H])c1-c1c([2H])c([2H])c2c3c(c([2H])c([2H])c([2H])c13)-c1ccccc1O2 ; [2H]c1c([2H])c2c3c(c([2H])c([2H])c(-c4cccc5c6ccc7ccccc7c6n(-c6ccccc6)c45)c3c1[2H])Oc1ccccc1-2 ; [2H]c1c([2H])c2c3c(c([2H])c([2H])c(-c4ccc5c(c4)c4ccc6ccccc6c4n5-c4ccccc4)c3c1[2H])Oc1ccccc1-2 ; [2H]c1c([2H])c2c3c(c([2H])c([2H])c(-c4cccc5c4c4ccc6ccccc6c4n5-c4ccccc4)c3c1[2H])Oc1ccccc1-2]

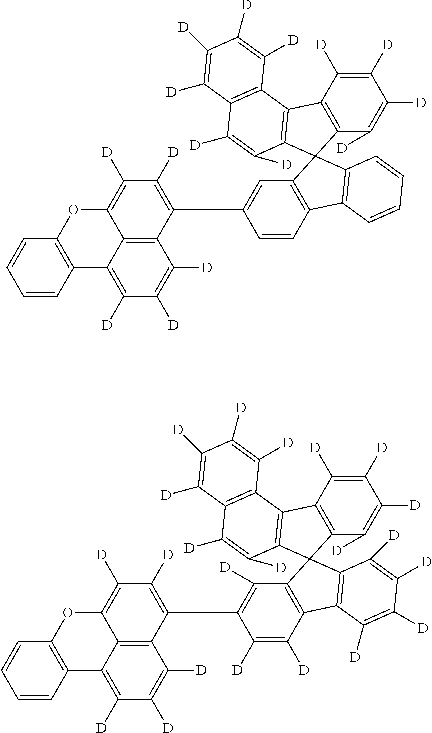 [SMILES: [2H]c1c([2H])c([2H])c2c(c1[2H])-c1c(c([2H])c([2H])c3c([2H])c([2H])c([2H])c([2H])c13)C21c2ccccc2-c2ccc(-c3c([2H])c([2H])c4c5c(c([2H])c([2H])c([2H])c35)-c3ccccc3O4)cc21 ; [2H]c1c([2H])c([2H])c2c(c1[2H])-c1c([2H])c([2H])c(-c3c([2H])c([2H])c4c5c(c([2H])c([2H])c([2H])c35)-c3ccccc3O4)c([2H])c1C21c2c([2H])c([2H])c([2H])c([2H])c2-c2c1c([2H])c([2H])c1c([2H])c([2H])c([2H])c([2H])c21]

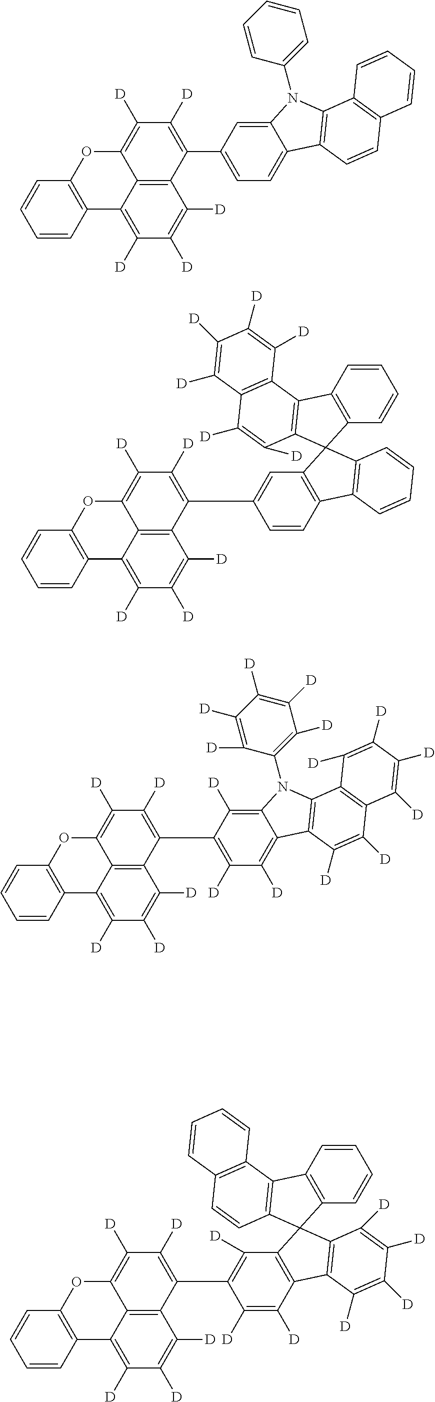

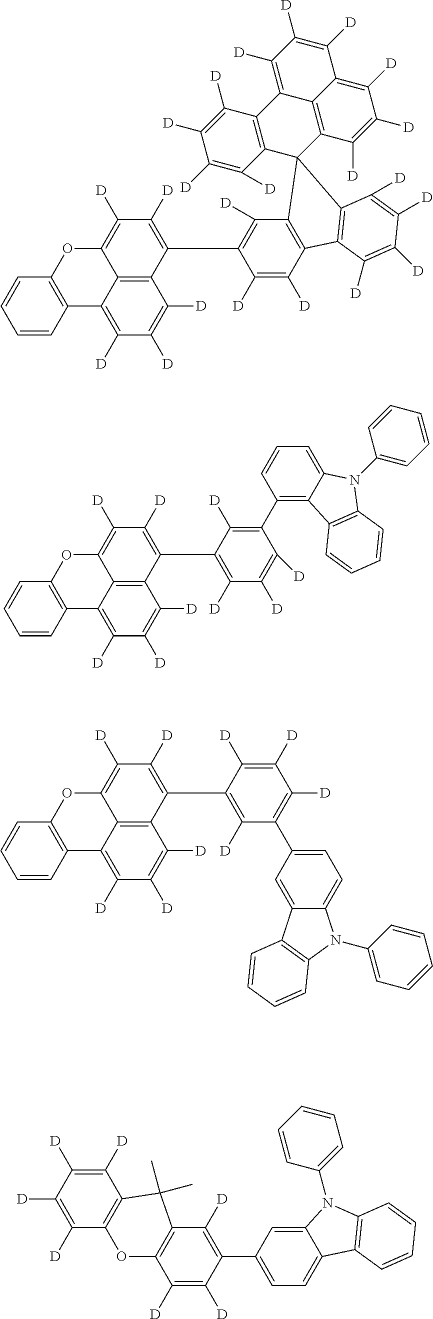

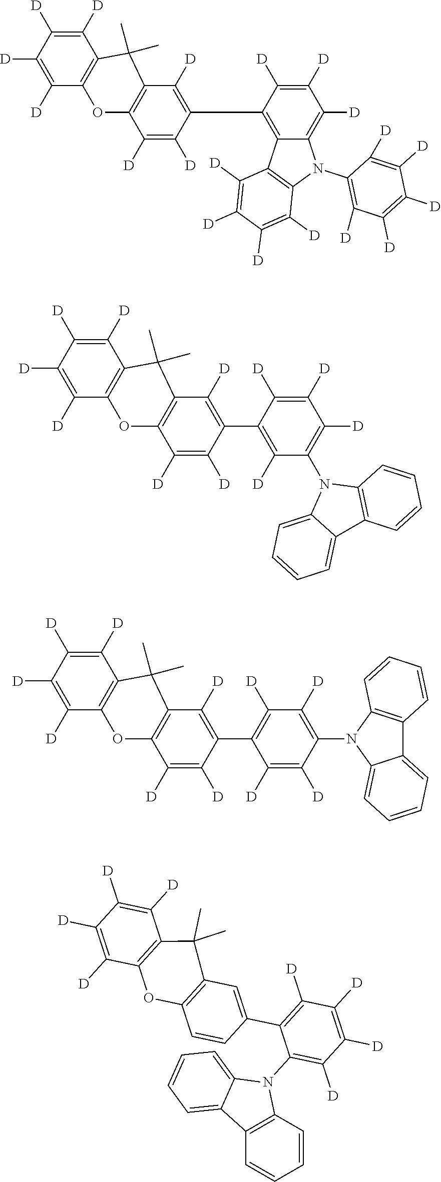

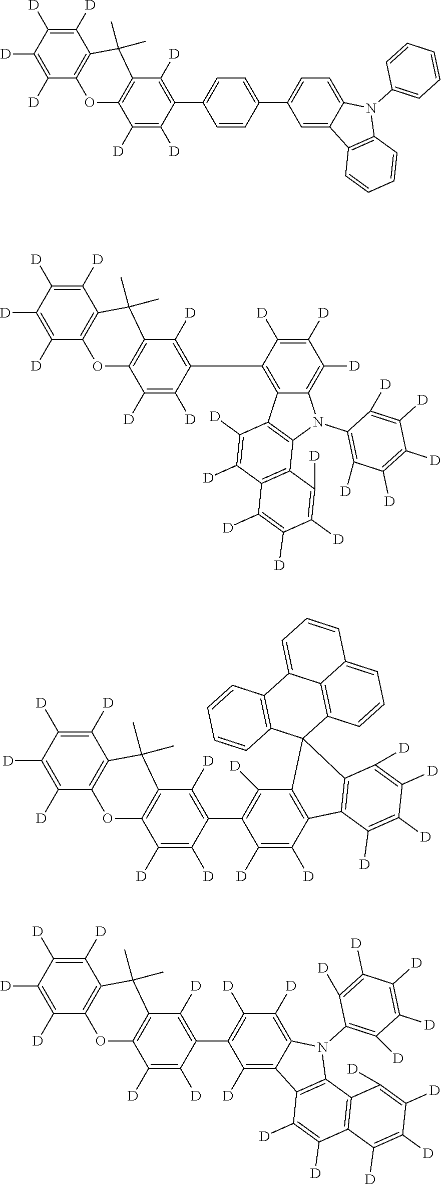

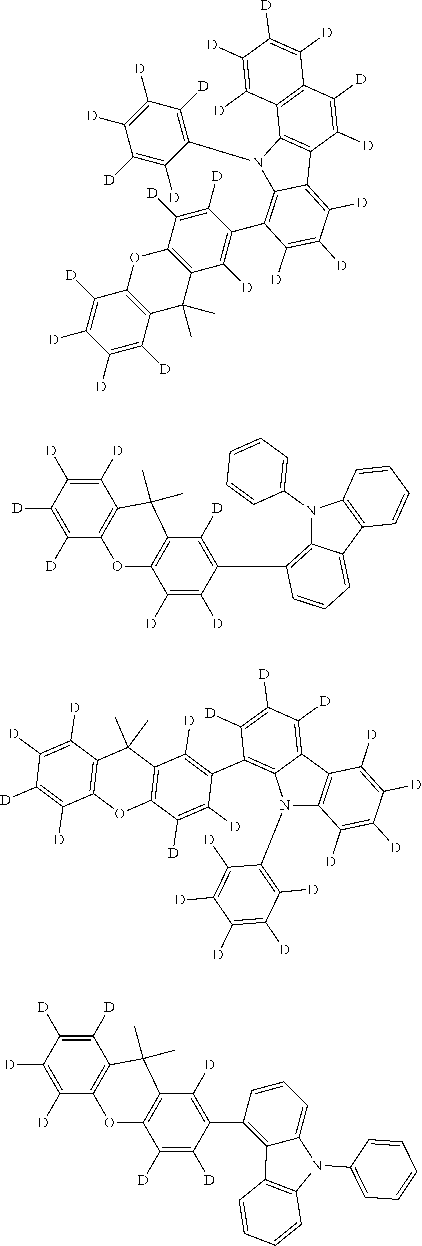

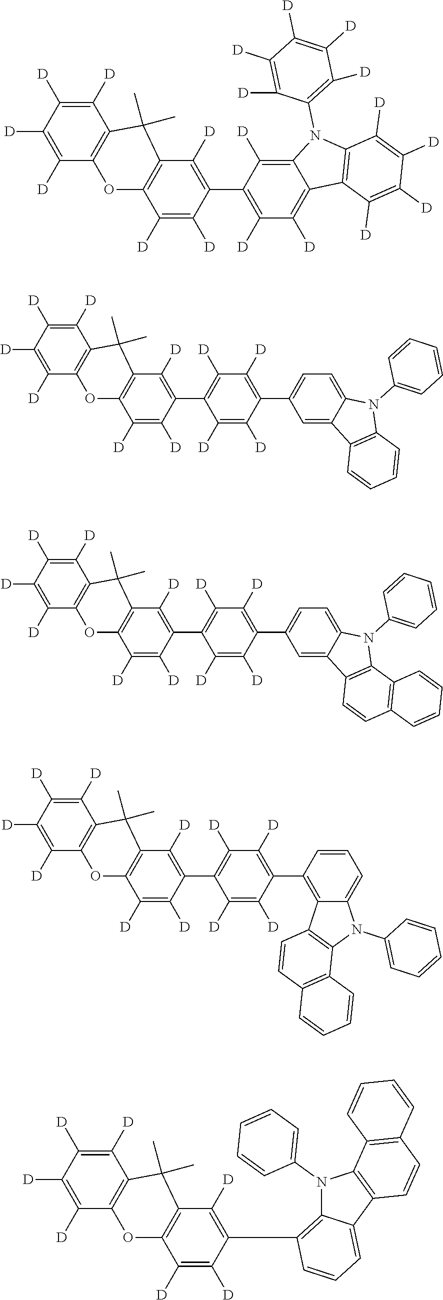

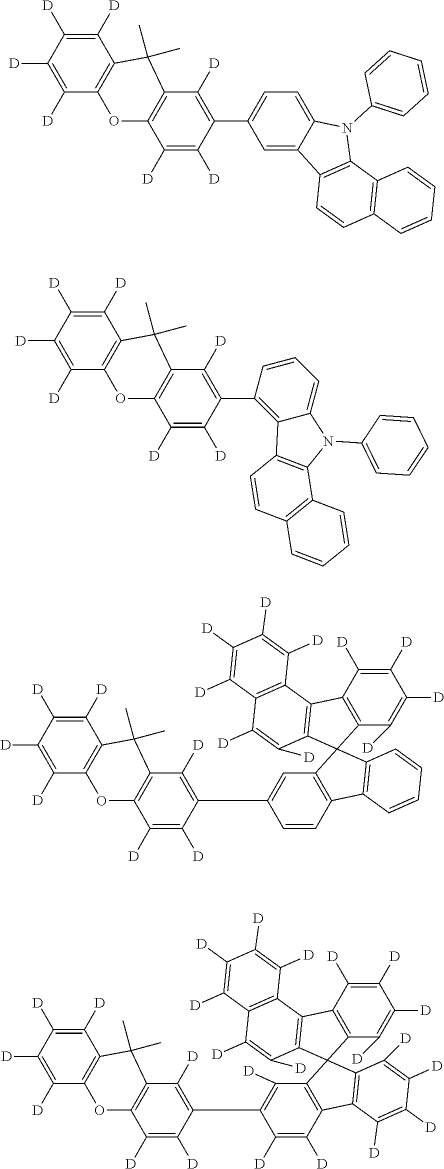

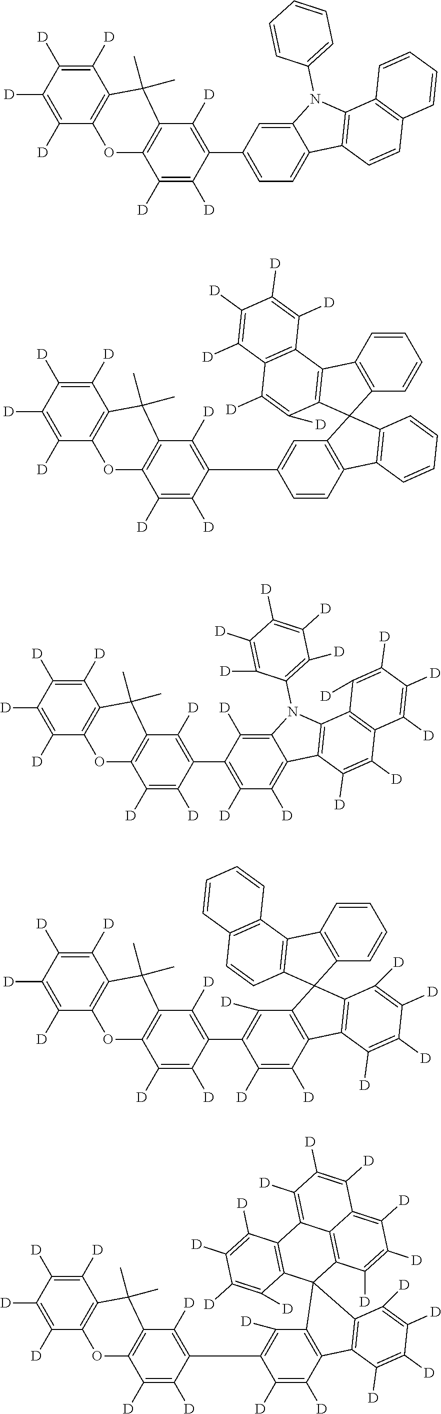

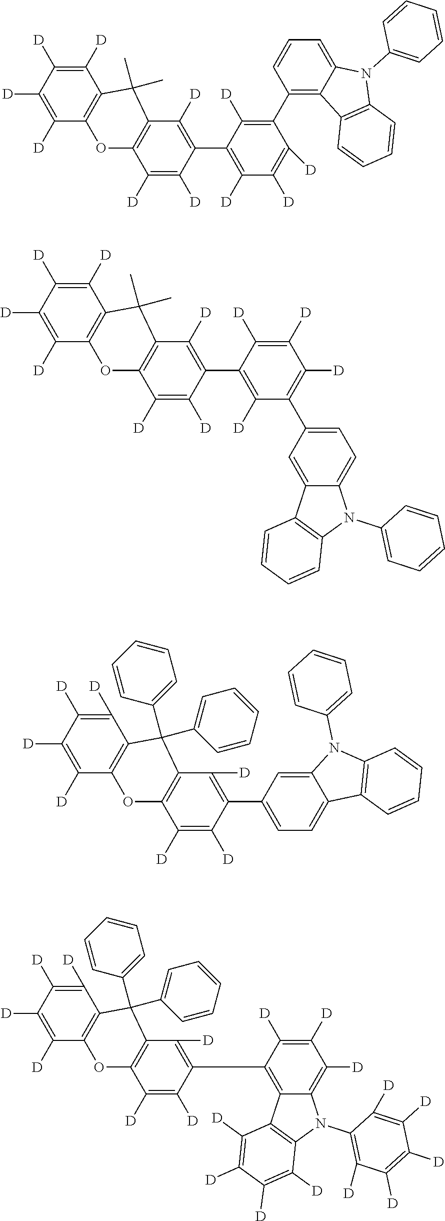

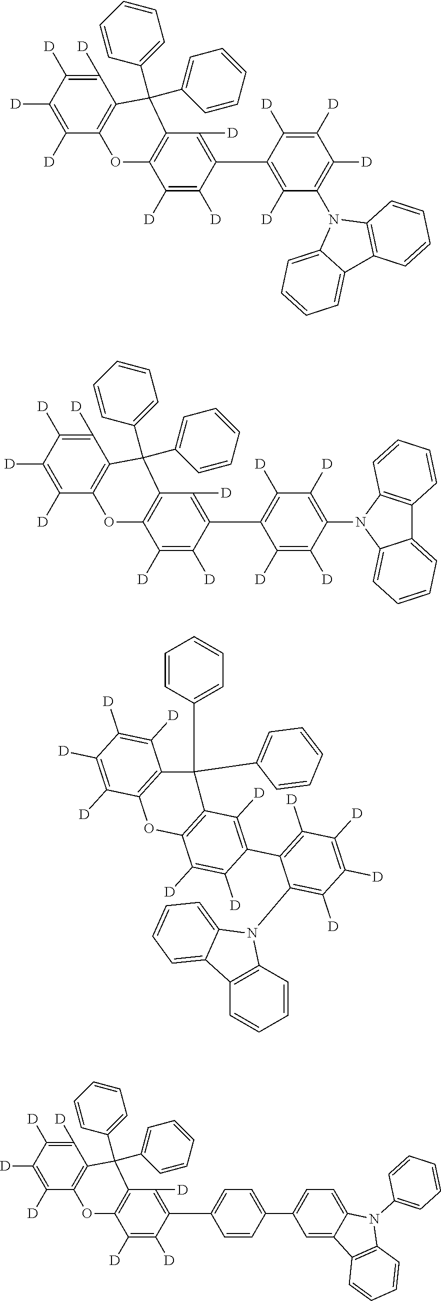

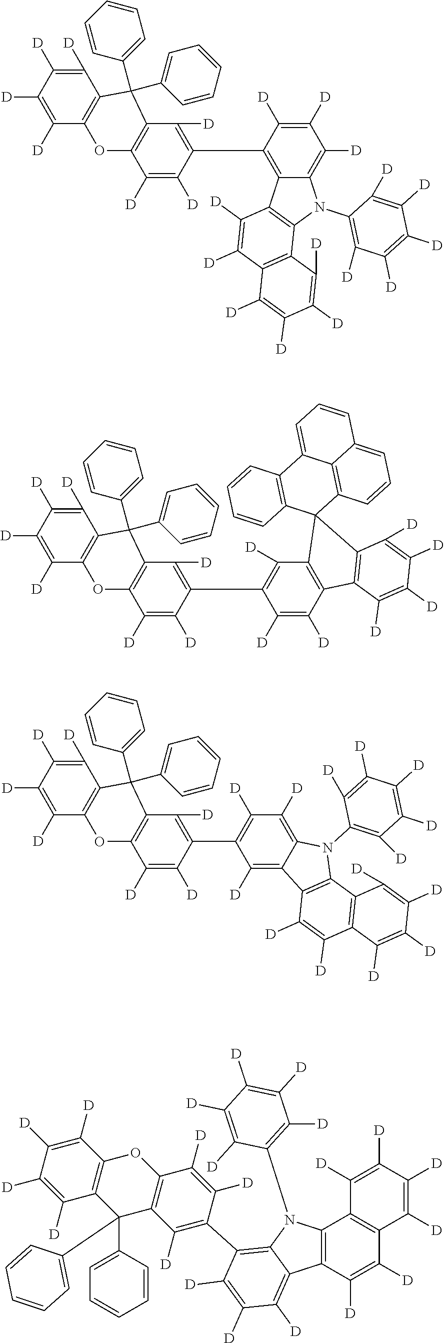

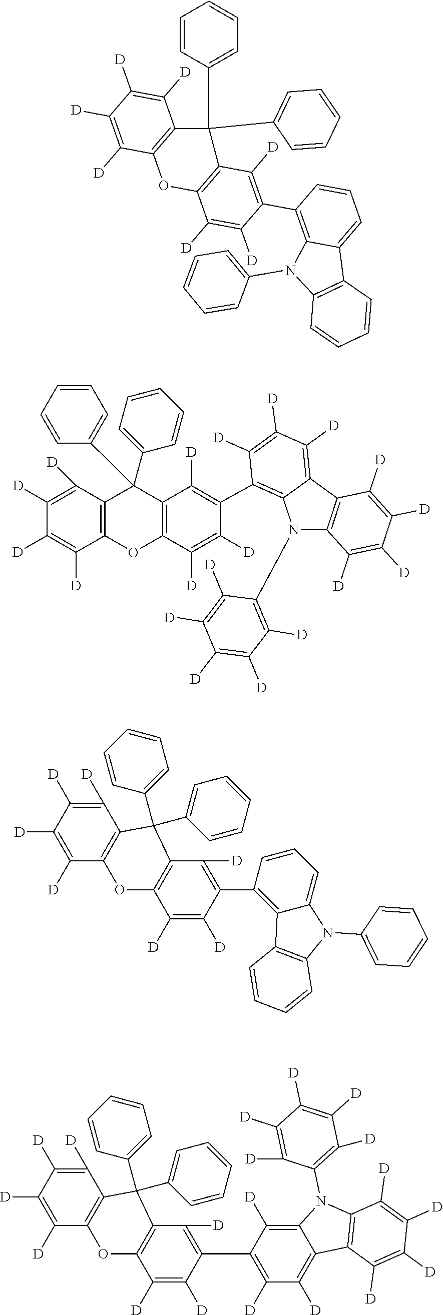

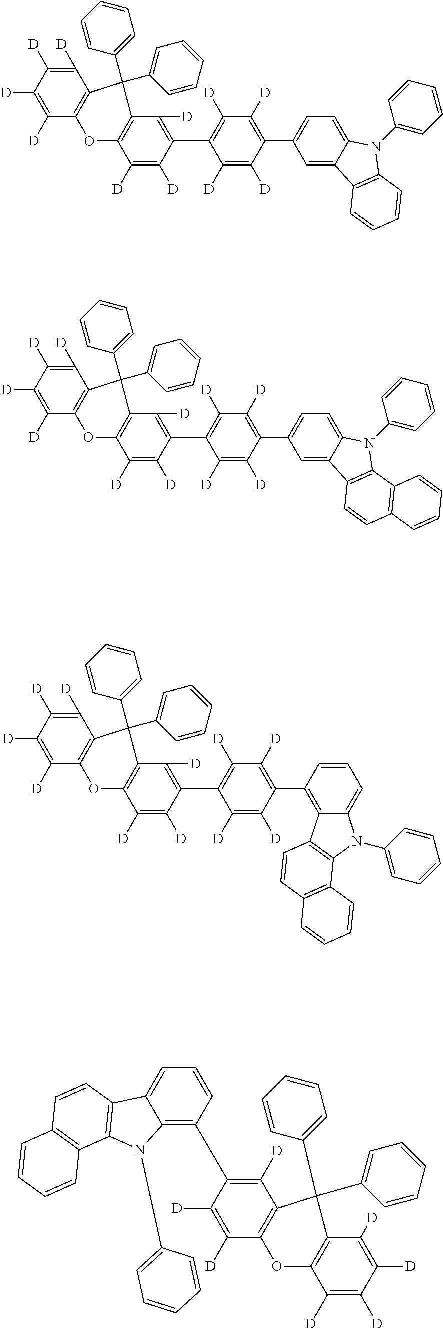

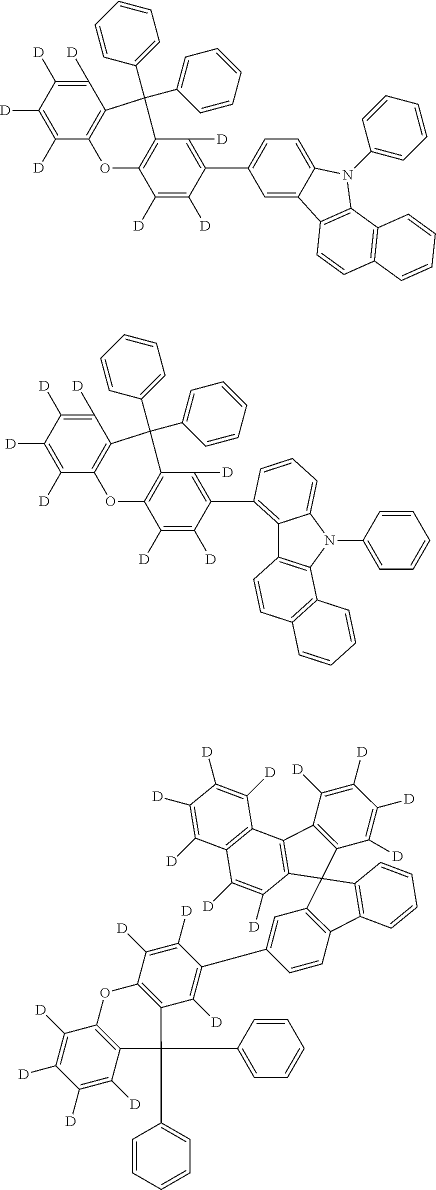 [SMILES: [2H]c1c([2H])c([2H])c2c(c1[2H])Oc1c([2H])c([2H])c(-c3ccc4c(c3)c3ccc5ccccc5c3n4-c3ccccc3)c([2H])c1C2(c1ccccc1)c1ccccc1 ; [2H]c1c([2H])c([2H])c2c(c1[2H])Oc1c([2H])c([2H])c(-c3cccc4c3c3ccc5ccccc5c3n4-c3ccccc3)c([2H])c1C2(c1ccccc1)c1ccccc1 ; [2H]c1c([2H])c([2H])c2c(c1[2H])Oc1c([2H])c([2H])c(-c3ccc4c(c3)C3(c5ccccc5-4)c4c([2H])c([2H])c([2H])c([2H])c4-c4c3c([2H])c([2H])c3c([2H])c([2H])c([2H])c([2H])c43)c([2H])c1C2(c1ccccc1)c1ccccc1]

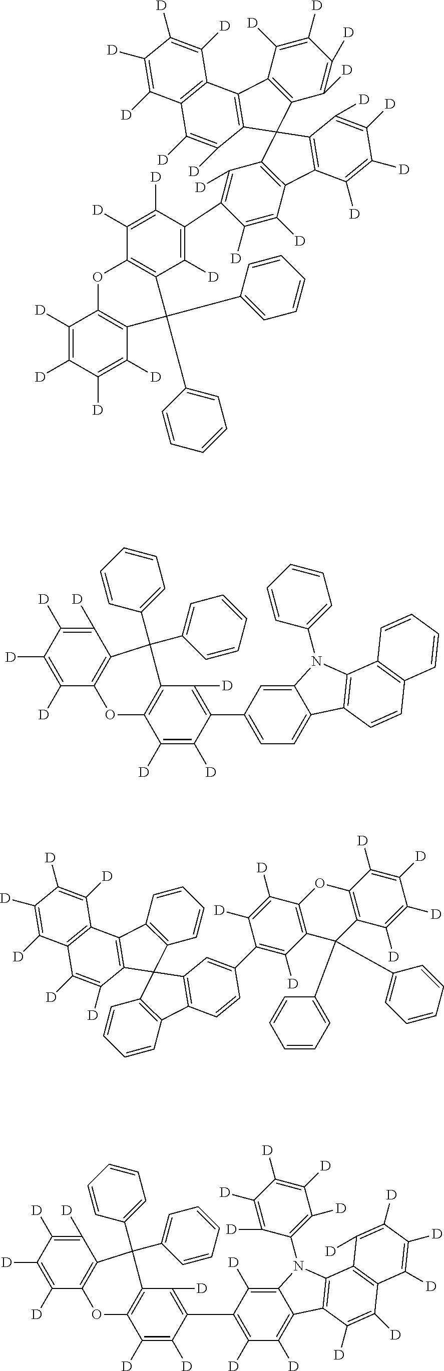

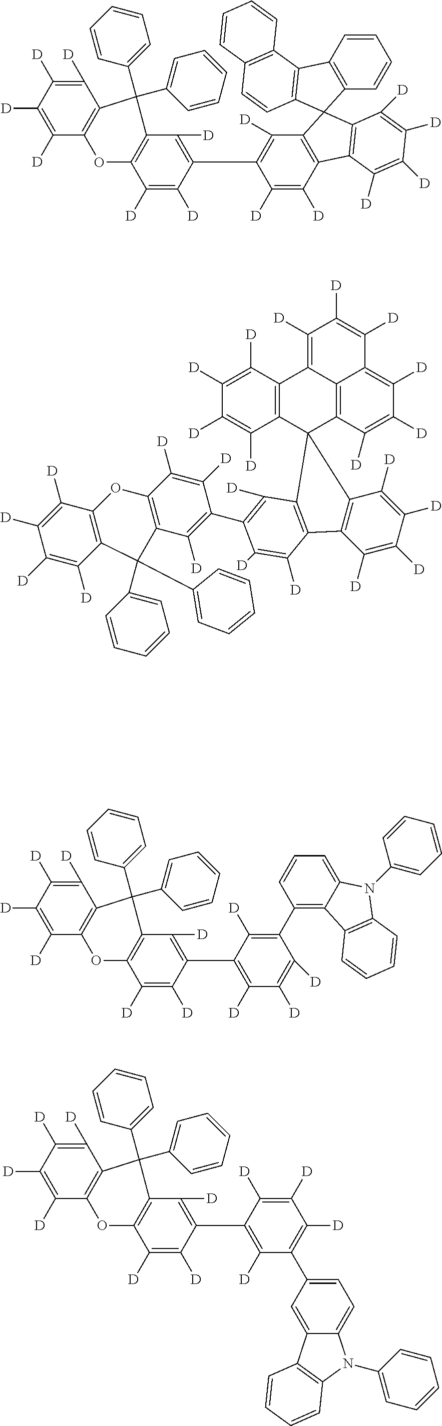

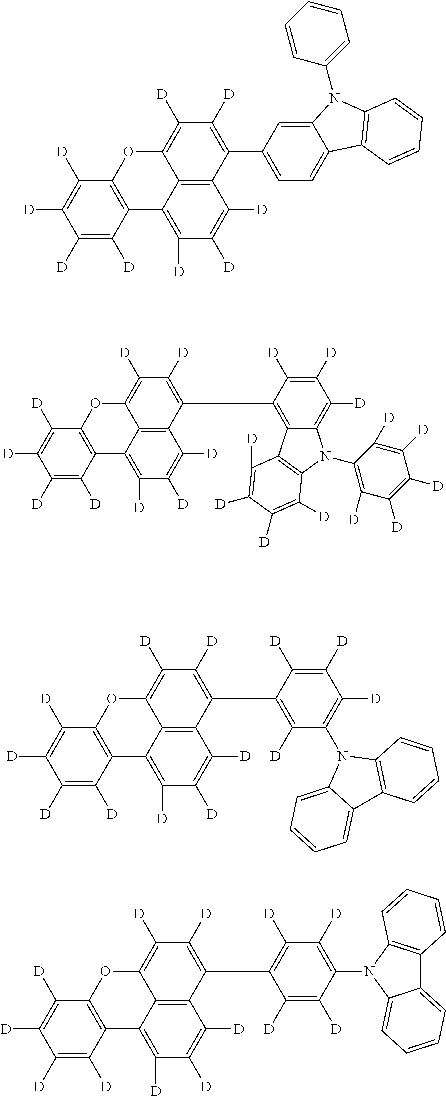

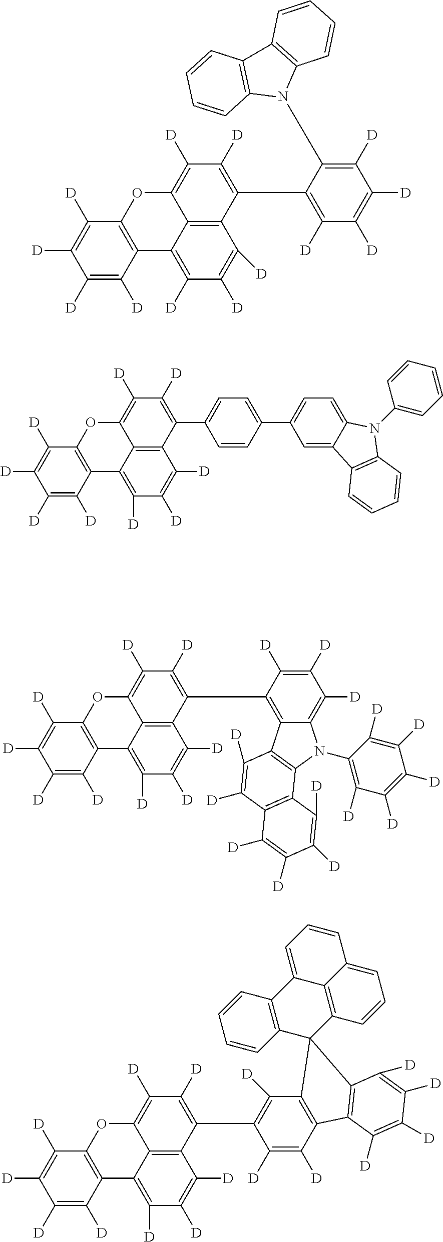

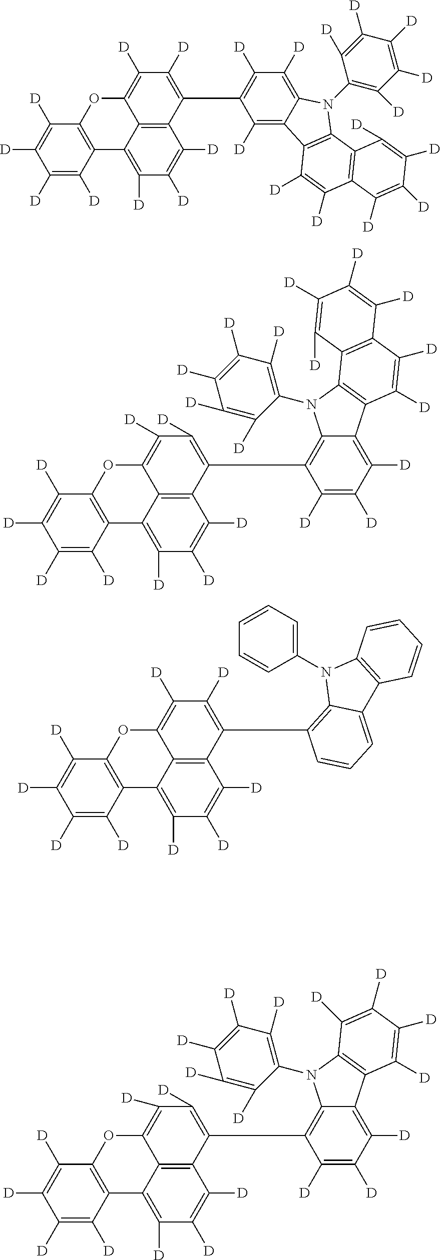

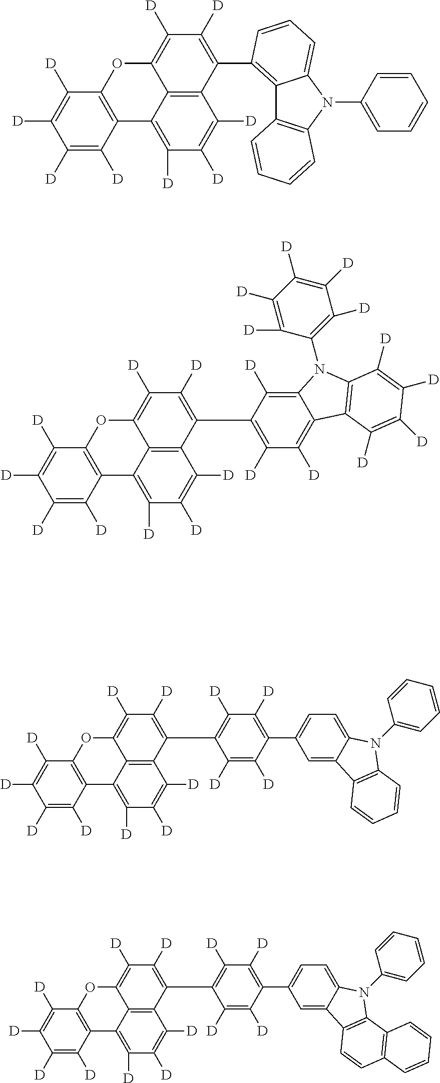

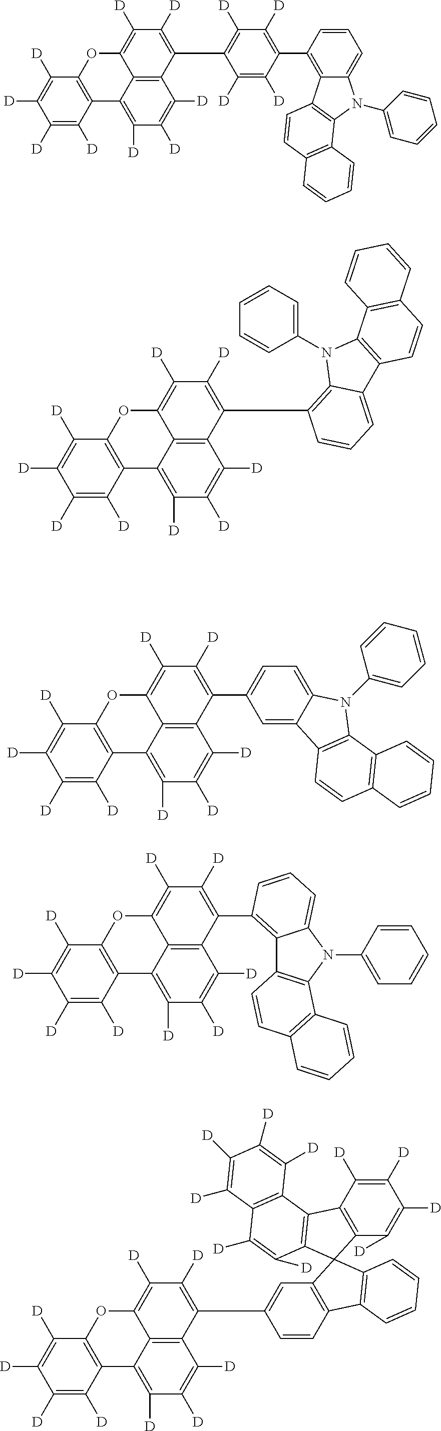

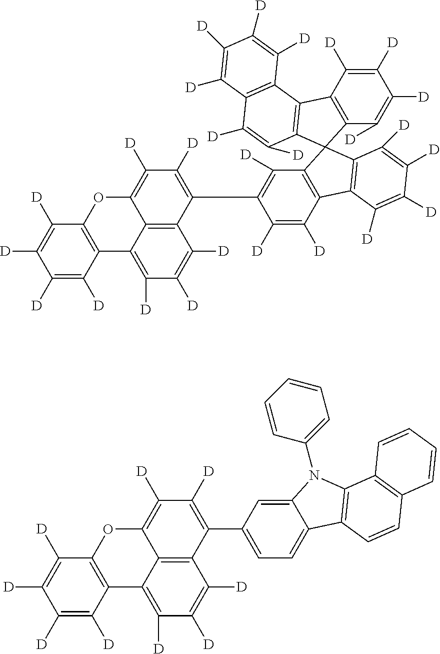 [SMILES: [2H]c1c([2H])c([2H])c2c(c1[2H])Oc1c([2H])c([2H])c(-c3c([2H])c([2H])c4c(c3[2H])C3(c5c([2H])c([2H])c([2H])c([2H])c5-4)c4c([2H])c([2H])c([2H])c([2H])c4-c4c3c([2H])c([2H])c3c([2H])c([2H])c([2H])c([2H])c43)c3c([2H])c([2H])c([2H])c-2c13 ; [2H]c1c([2H])c([2H])c2c(c1[2H])Oc1c([2H])c([2H])c(-c3ccc4c5ccc6ccccc6c5n(-c5ccccc5)c4c3)c3c([2H])c([2H])c([2H])c-2c13]

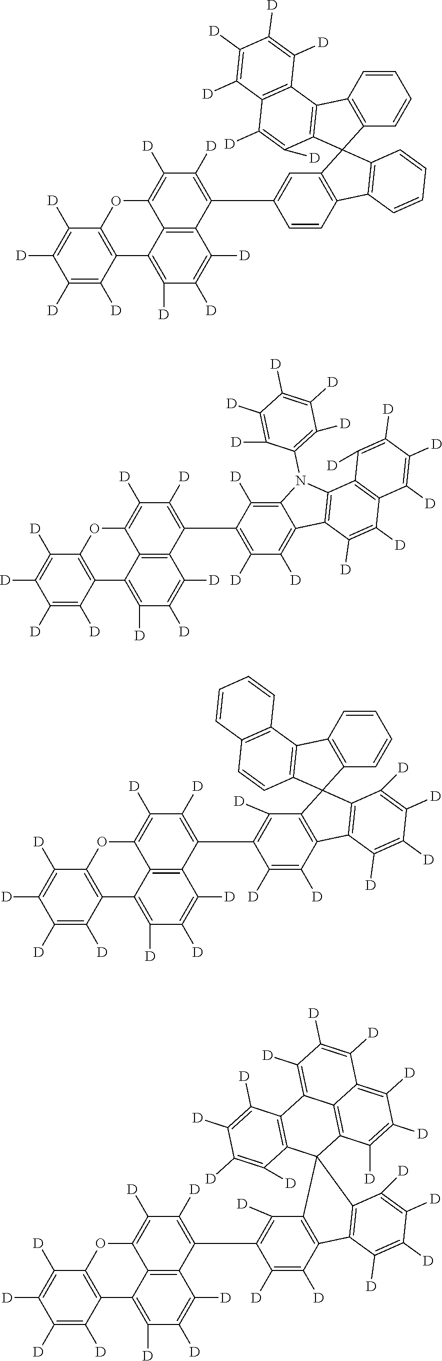

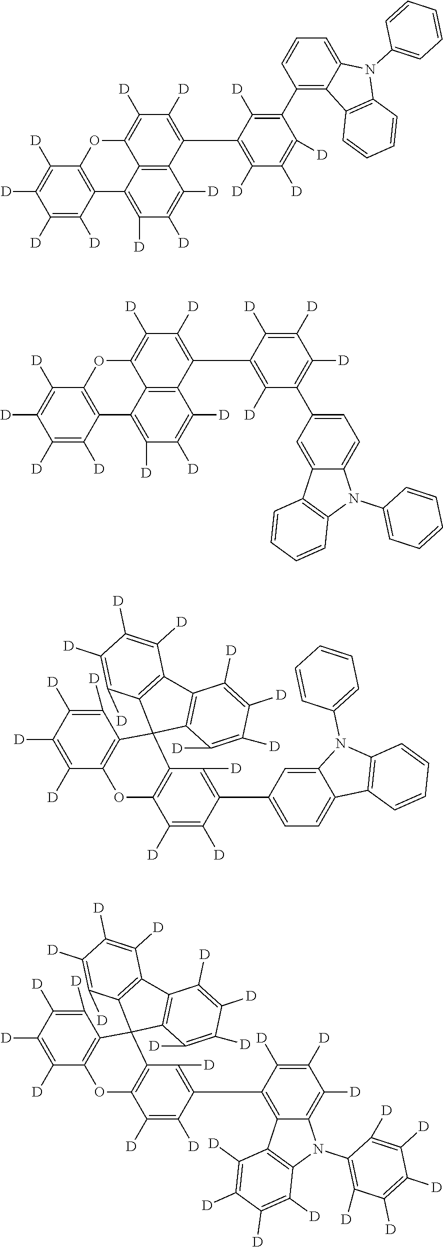

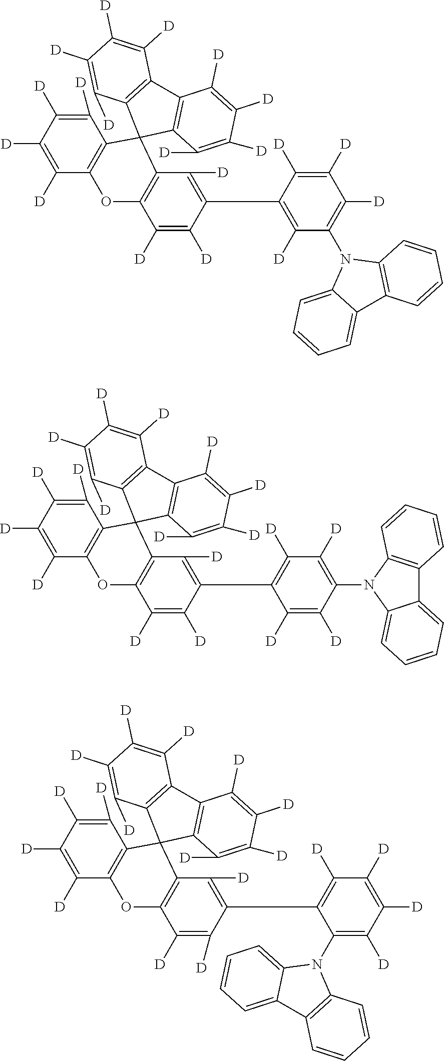

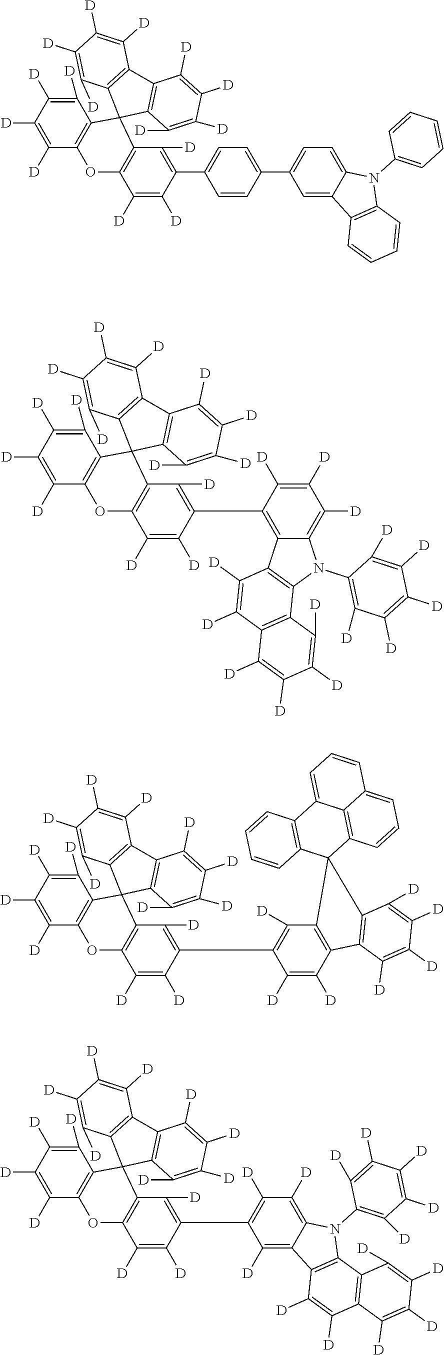

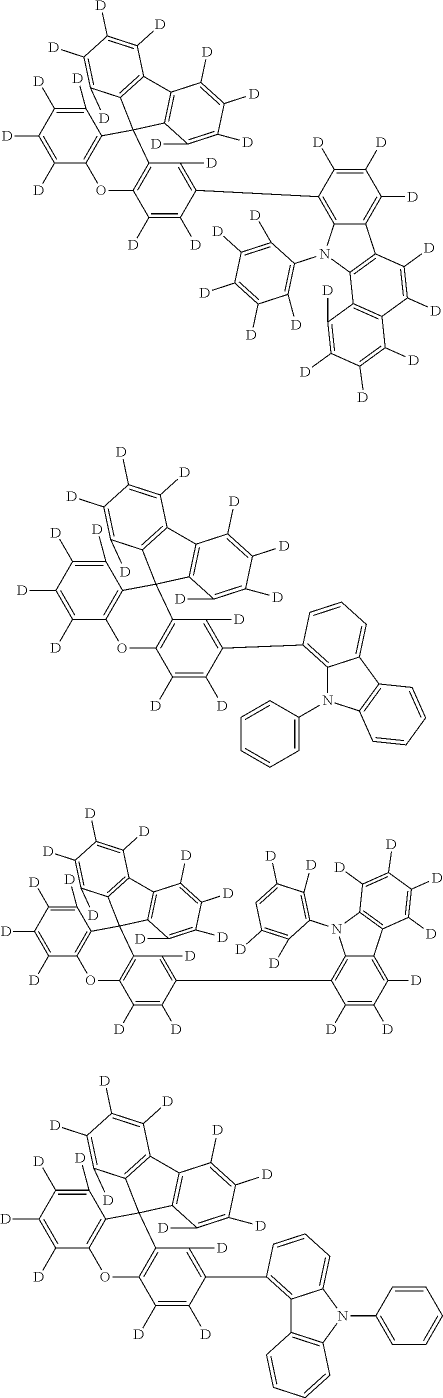

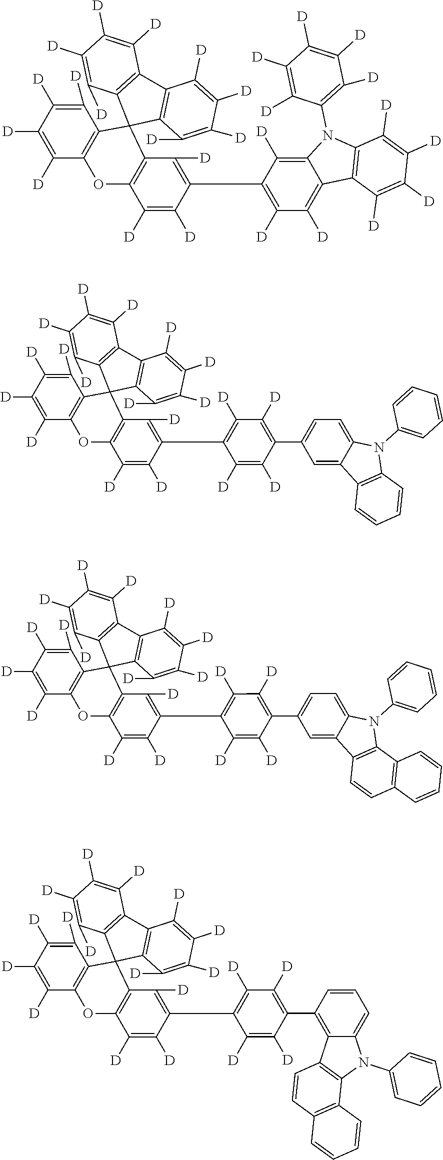

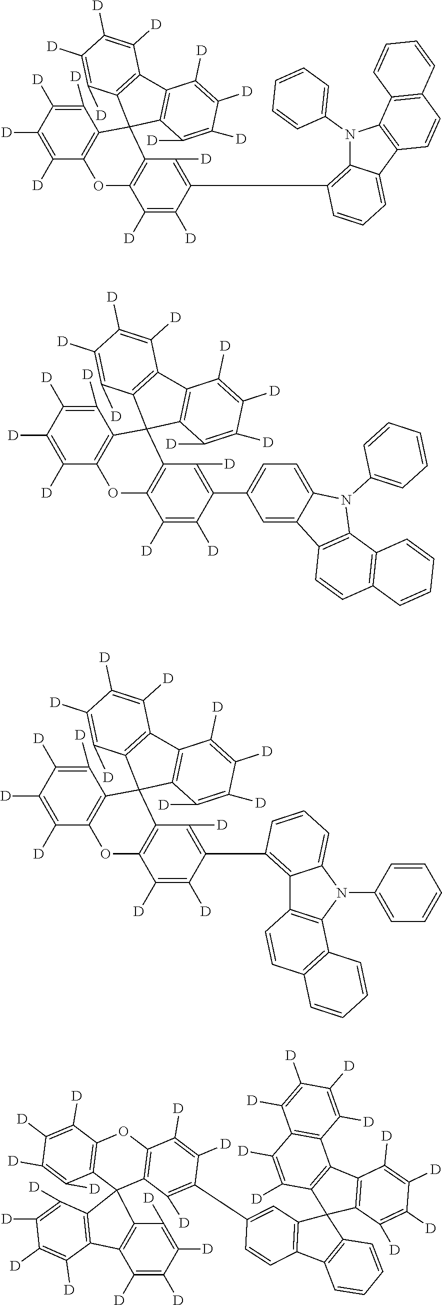

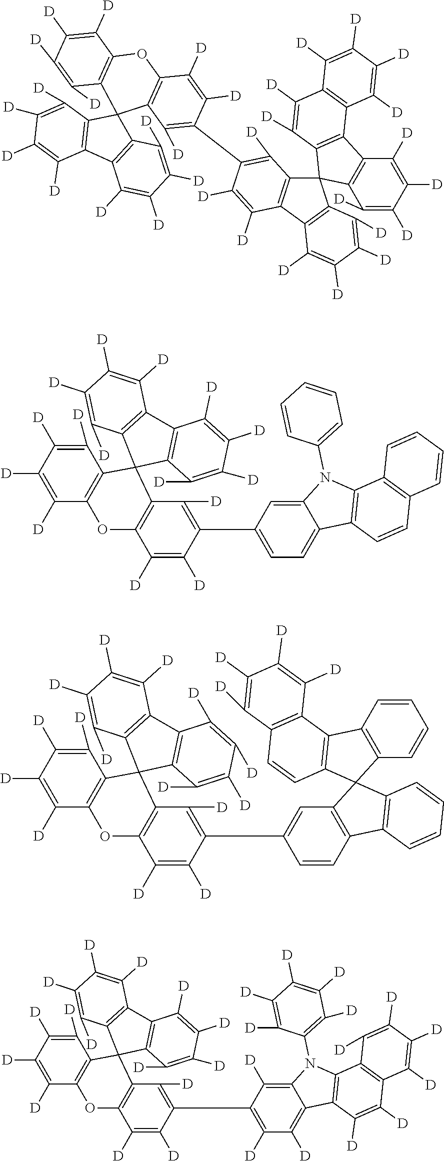

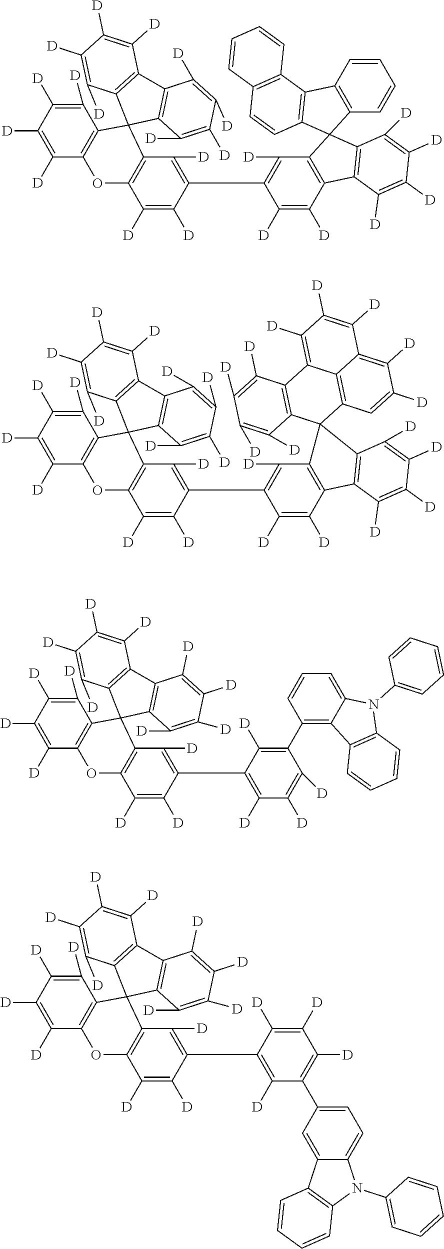

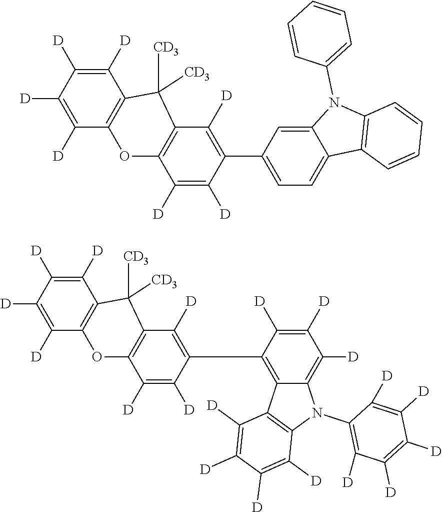 [SMILES: [2H]c1c([2H])c([2H])c2c(c1[2H])Oc1c([2H])c([2H])c(-c3ccc4c5ccccc5n(-c5ccccc5)c4c3)c([2H])c1C2(C([2H])([2H])[2H])C([2H])([2H])[2H] ; [2H]c1c([2H])c([2H])c(-n2c3c([2H])c([2H])c([2H])c([2H])c3c3c(-c4c([2H])c([2H])c5c(c4[2H])C(C([2H])([2H])[2H])(C([2H])([2H])[2H])c4c([2H])c([2H])c([2H])c([2H])c4O5)c([2H])c([2H])c([2H])c32)c([2H])c1[2H]]

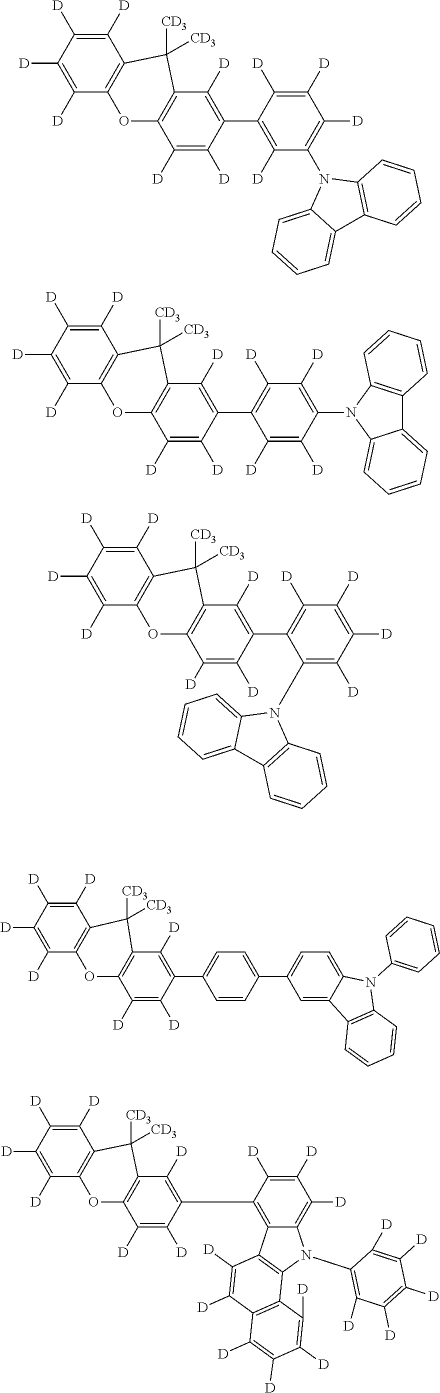

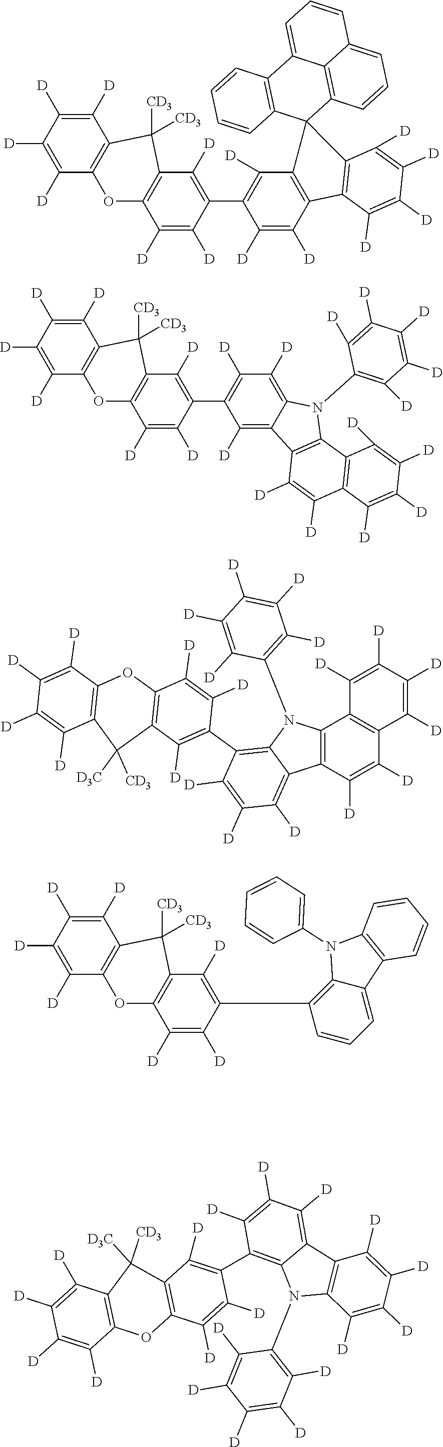

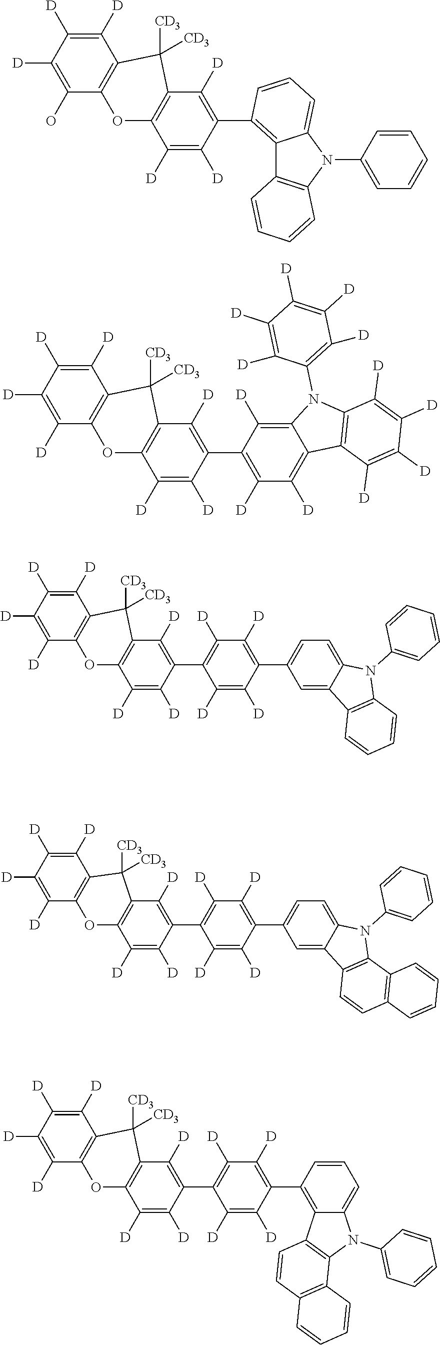

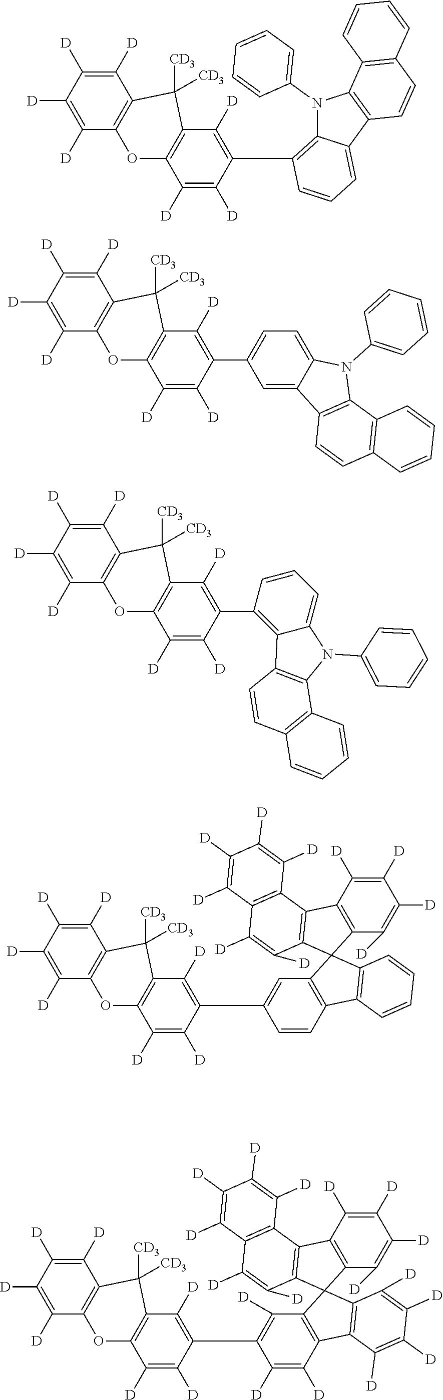

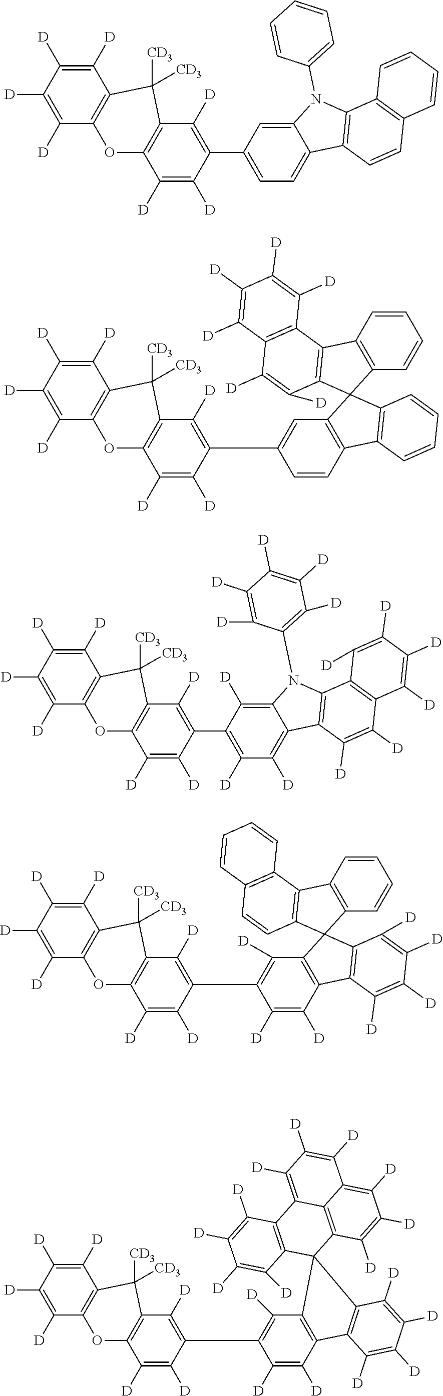

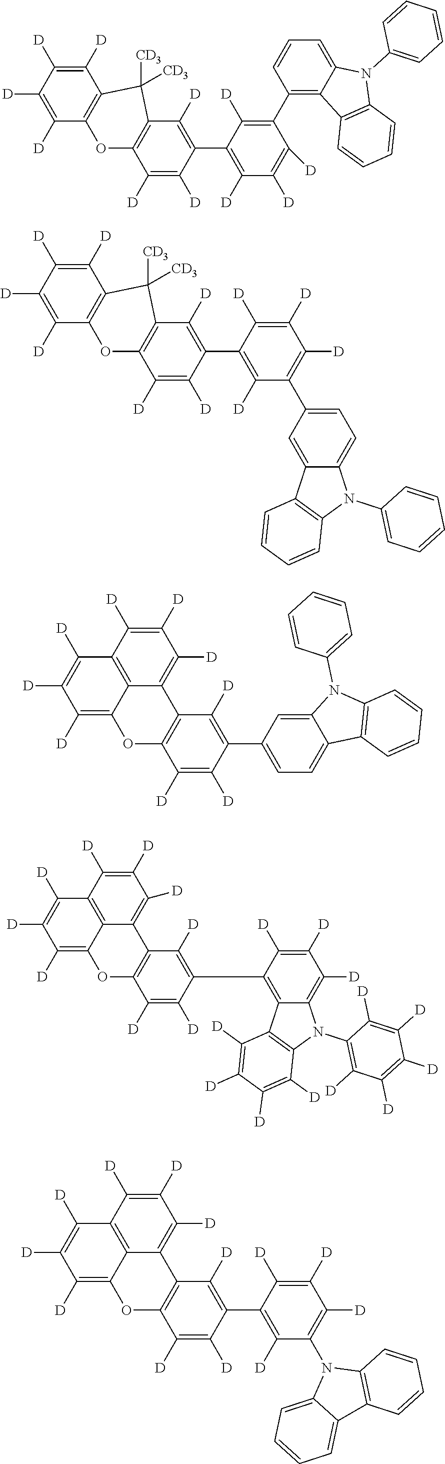

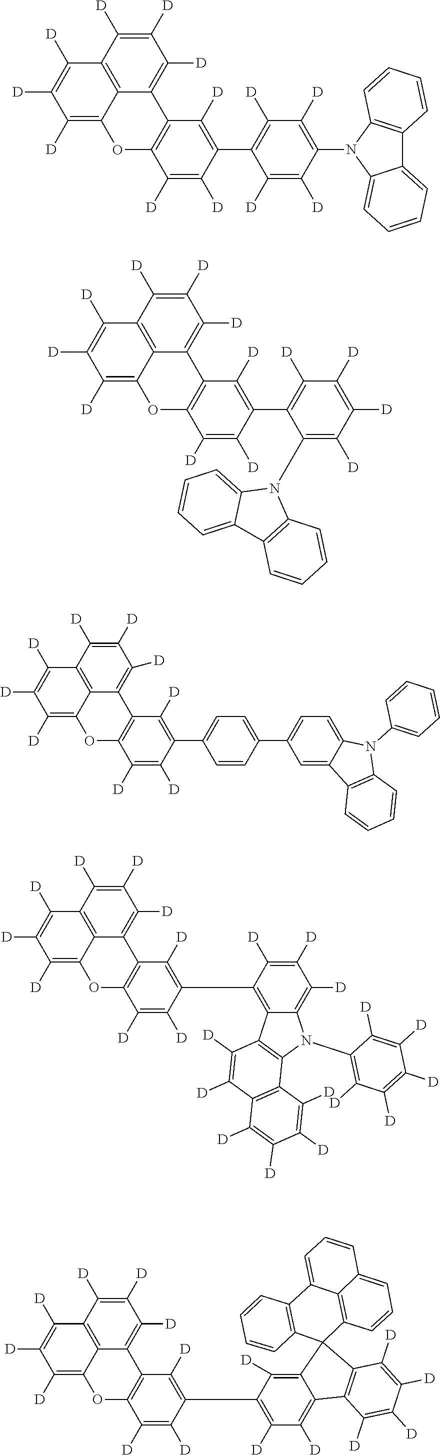

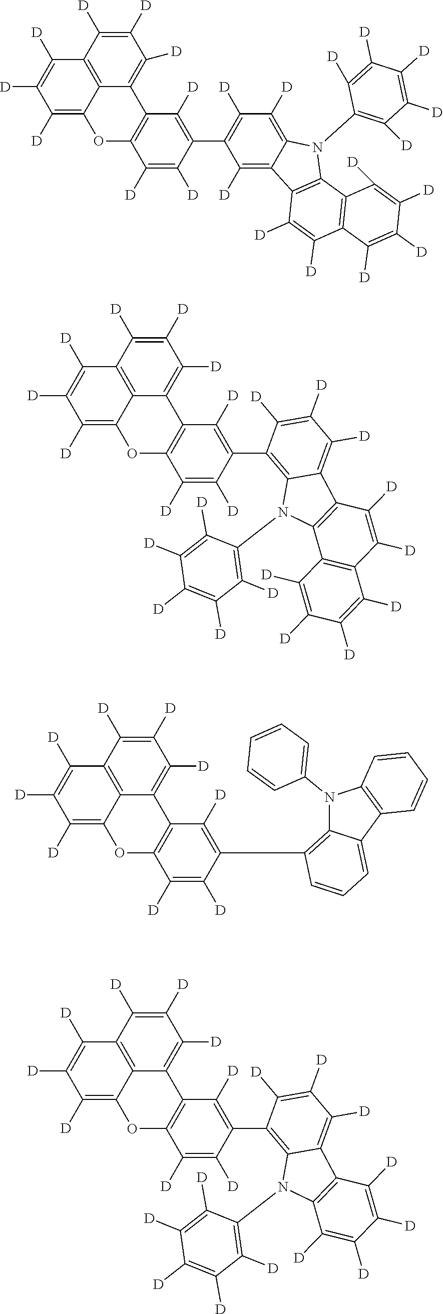

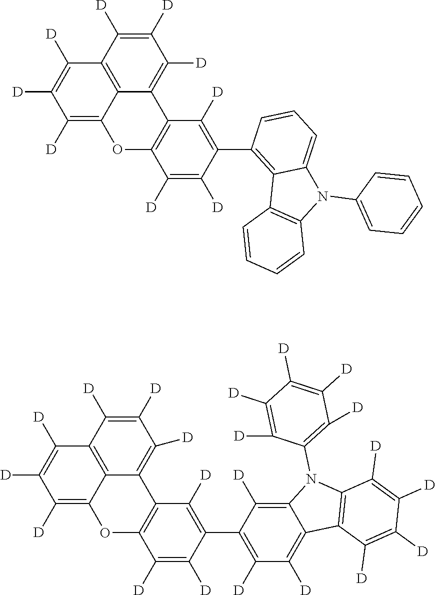 [SMILES: [2H]c1c([2H])c(-c2cccc3c2c2ccccc2n3-c2ccccc2)c([2H])c2c1Oc1c([2H])c([2H])c([2H])c3c([2H])c([2H])c([2H])c-2c13 ; [2H]c1c([2H])c([2H])c(-n2c3c([2H])c([2H])c([2H])c([2H])c3c3c([2H])c([2H])c(-c4c([2H])c([2H])c5c(c4[2H])-c4c([2H])c([2H])c([2H])c6c([2H])c([2H])c([2H])c(c46)O5)c([2H])c32)c([2H])c1[2H]]

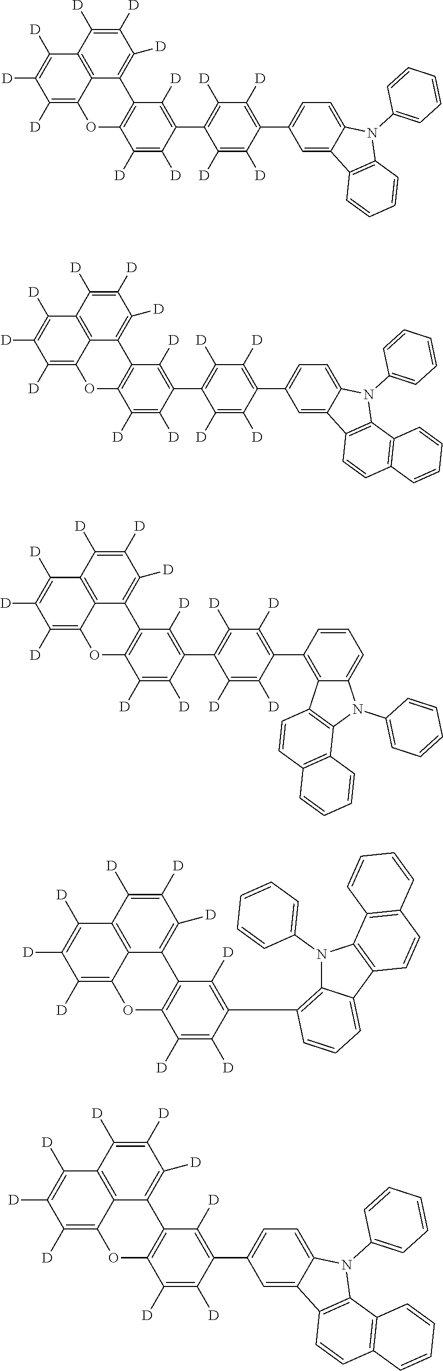

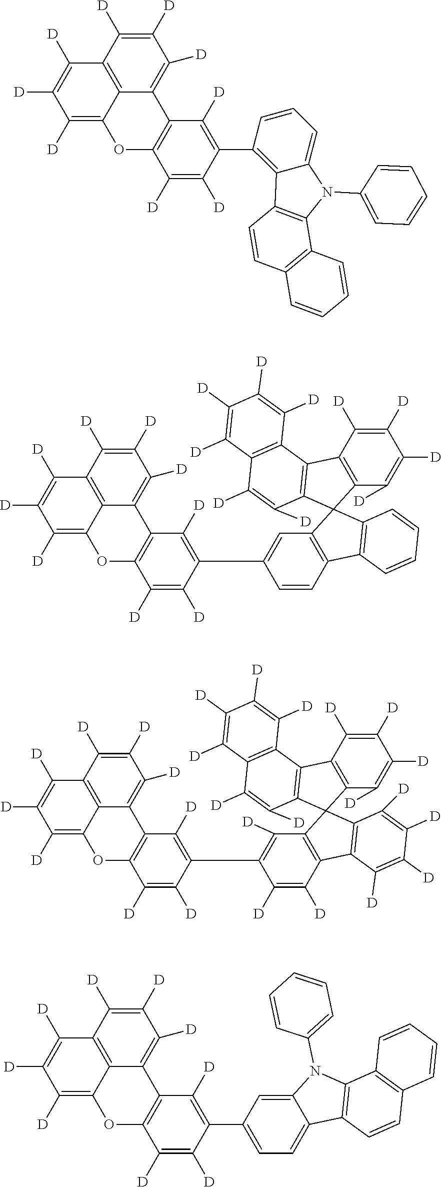

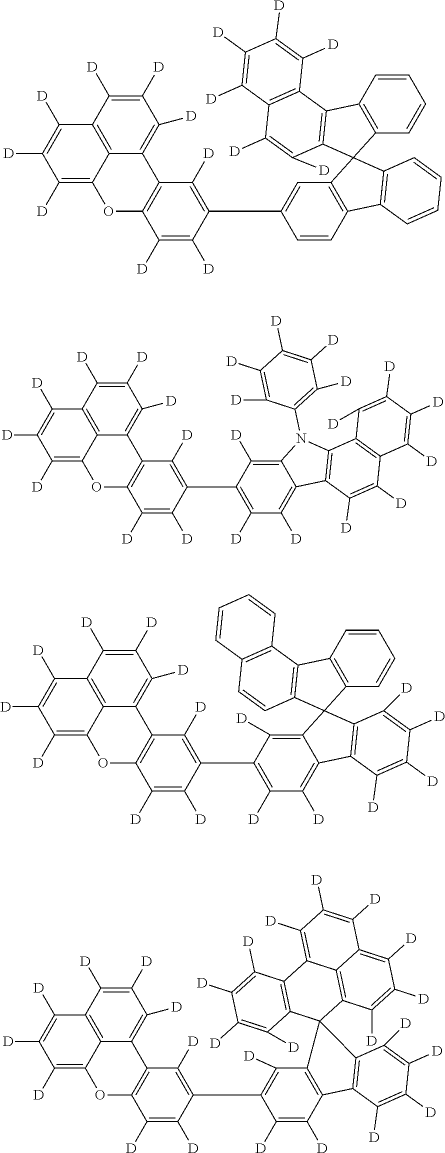

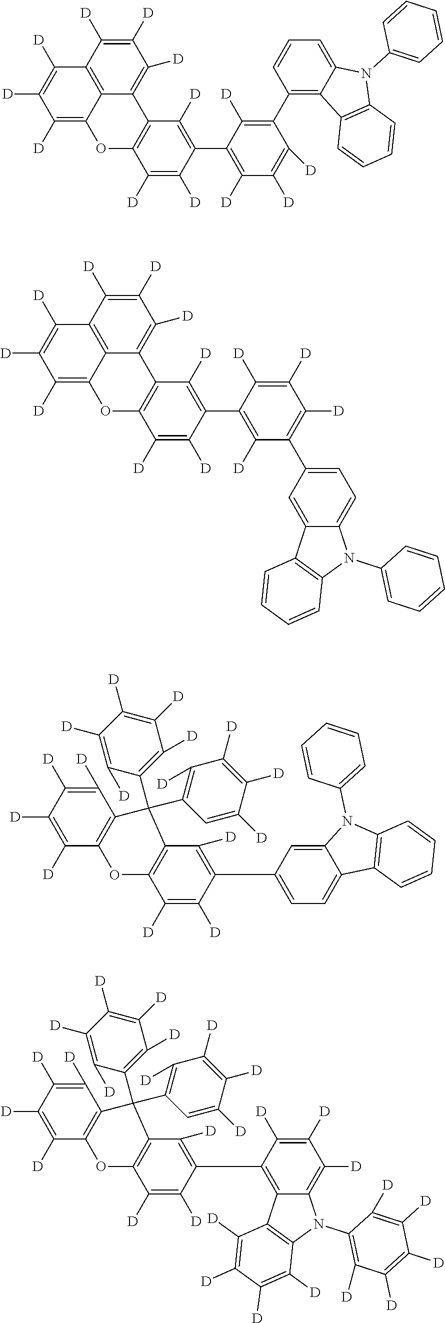

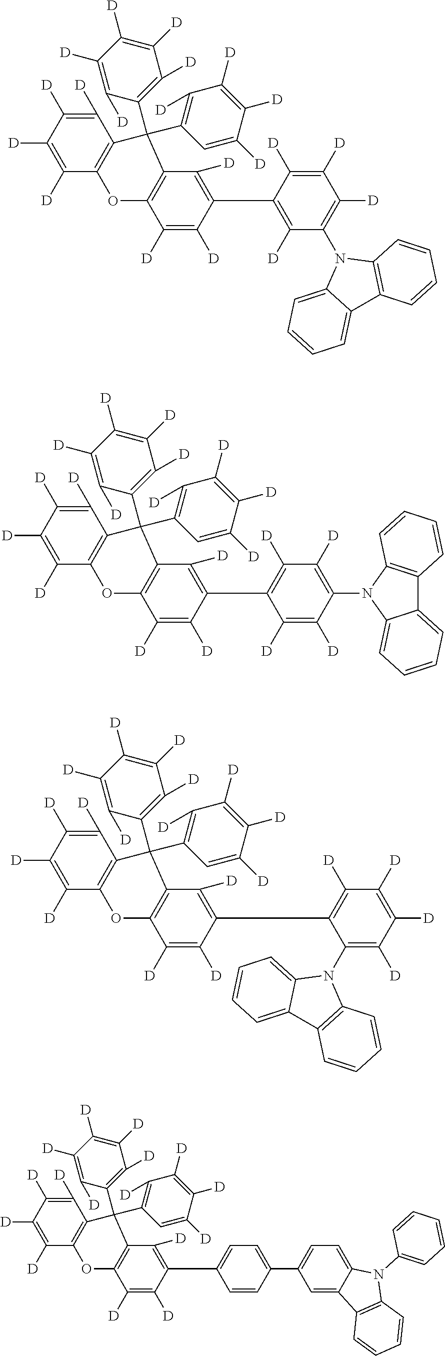

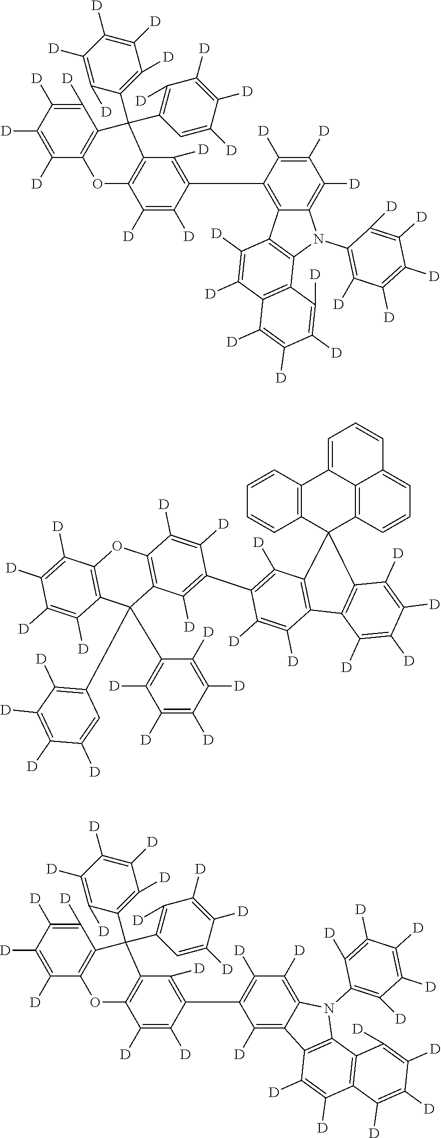

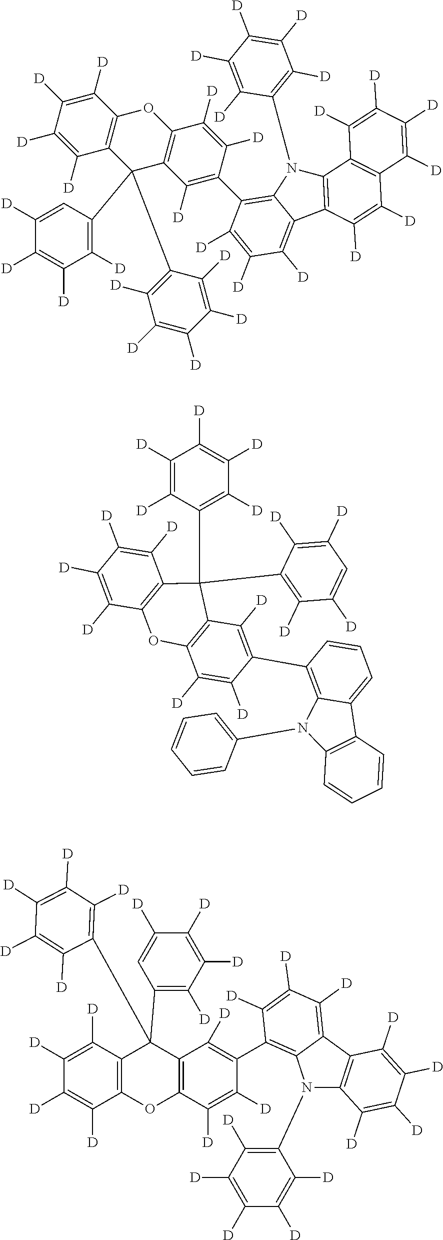

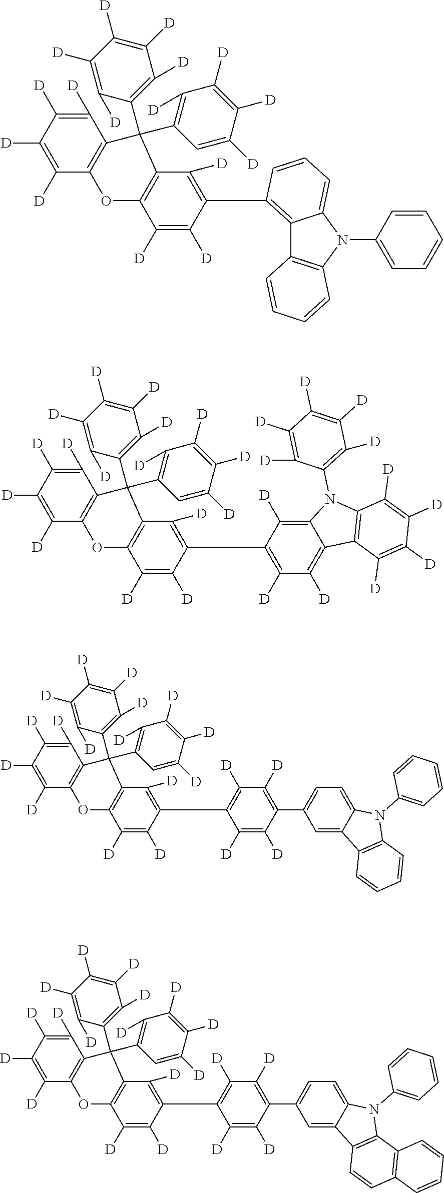

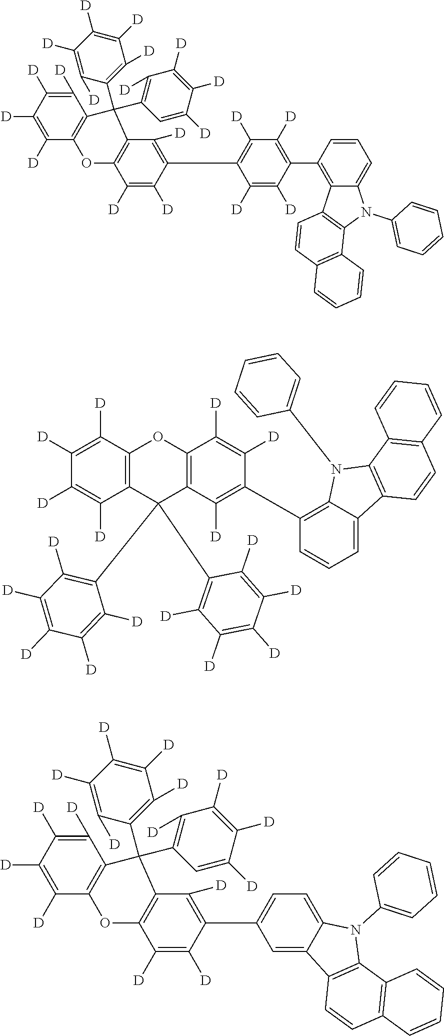

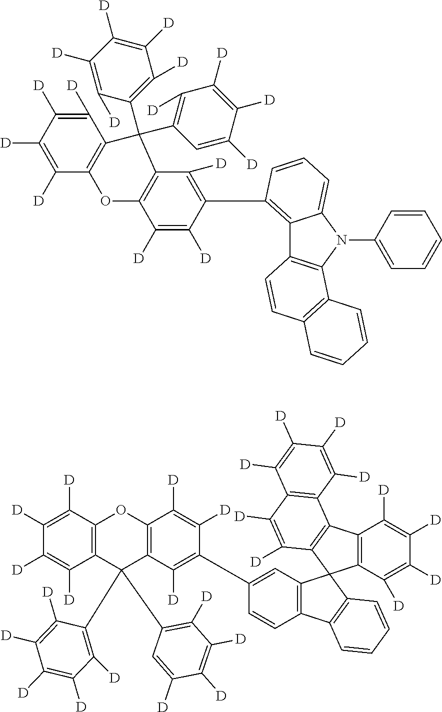 [SMILES: [2H]c1cc(C2(c3c([2H])c([2H])c([2H])c([2H])c3[2H])c3c([2H])c([2H])c([2H])c([2H])c3Oc3c([2H])c([2H])c(-c4cccc5c4c4ccc6ccccc6c4n5-c4ccccc4)c([2H])c32)c([2H])c([2H])c1[2H] ; [2H]c1cc(C2(c3c([2H])c([2H])c([2H])c([2H])c3[2H])c3c([2H])c([2H])c([2H])c([2H])c3Oc3c([2H])c([2H])c(-c4ccc5c(c4)C4(c6ccccc6-5)c5c([2H])c([2H])c([2H])c([2H])c5-c5c4c([2H])c([2H])c4c([2H])c([2H])c([2H])c([2H])c54)c([2H])c32)c([2H])c([2H])c1[2H]]

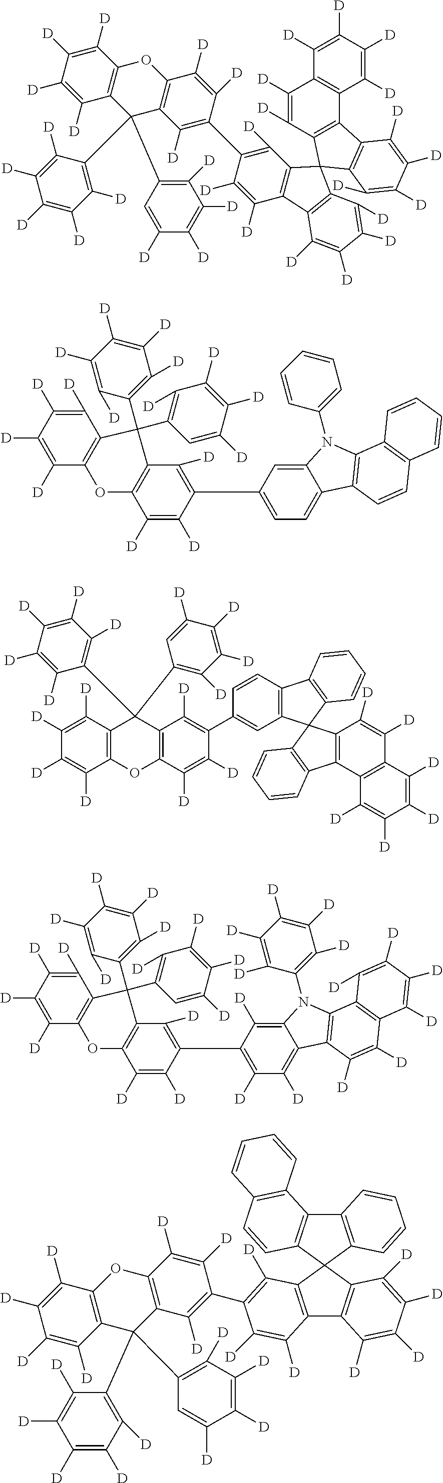

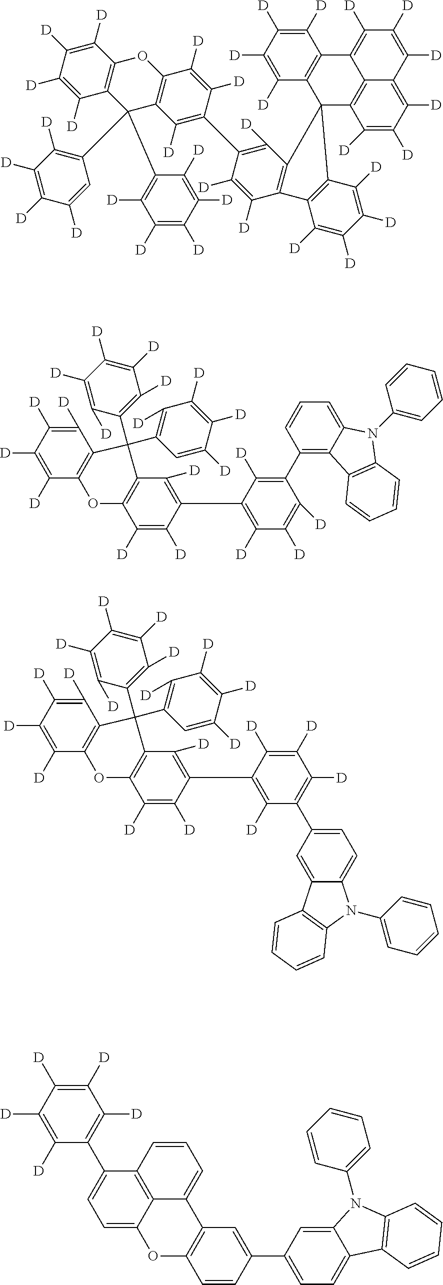

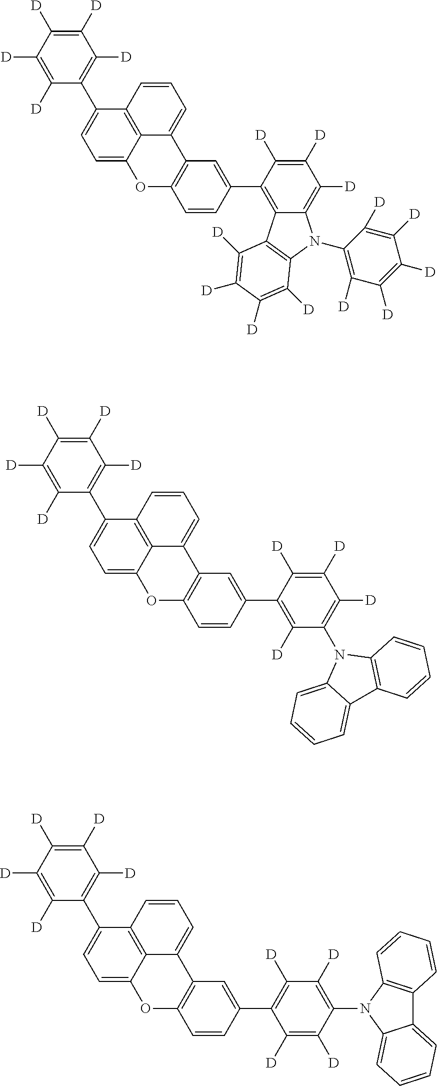 [SMILES: [2H]c1c([2H])c([2H])c(-c2ccc3c4c(cccc24)-c2cc(-c4c([2H])c([2H])c([2H])c5c4c4c([2H])c([2H])c([2H])c([2H])c4n5-c4c([2H])c([2H])c([2H])c([2H])c4[2H])ccc2O3)c([2H])c1[2H] ; [2H]c1c([2H])c([2H])c(-c2ccc3c4c(cccc24)-c2cc(-c4c([2H])c([2H])c([2H])c(-n5c6ccccc6c6ccccc65)c4[2H])ccc2O3)c([2H])c1[2H] ; [2H]c1c([2H])c([2H])c(-c2ccc3c4c(cccc24)-c2cc(-c4c([2H])c([2H])c(-n5c6ccccc6c6ccccc65)c([2H])c4[2H])ccc2O3)c([2H])c1[2H]]

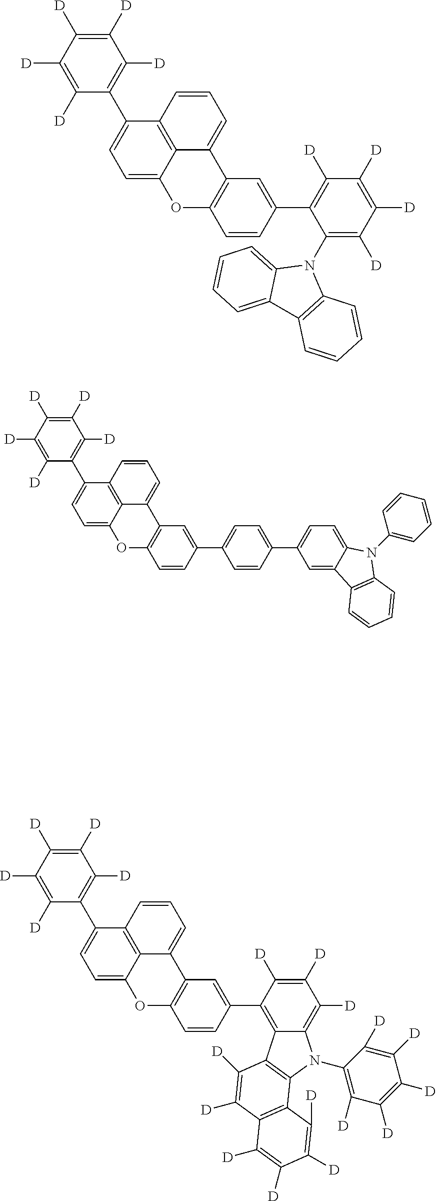 [SMILES: [2H]c1c([2H])c([2H])c(-c2ccc3c4c(cccc24)-c2cc(-c4c([2H])c([2H])c([2H])c([2H])c4-n4c5ccccc5c5ccccc54)ccc2O3)c([2H])c1[2H] ; [2H]c1c([2H])c([2H])c(-c2ccc3c4c(cccc24)-c2cc(-c4ccc(-c5ccc6c(c5)c5ccccc5n6-c5ccccc5)cc4)ccc2O3)c([2H])c1[2H] ; [2H]c1c([2H])c([2H])c(-c2ccc3c4c(cccc24)-c2cc(-c4c([2H])c([2H])c([2H])c5c4c4c([2H])c([2H])c6c([2H])c([2H])c([2H])c([2H])c6c4n5-c4c([2H])c([2H])c([2H])c([2H])c4[2H])ccc2O3)c([2H])c1[2H]]

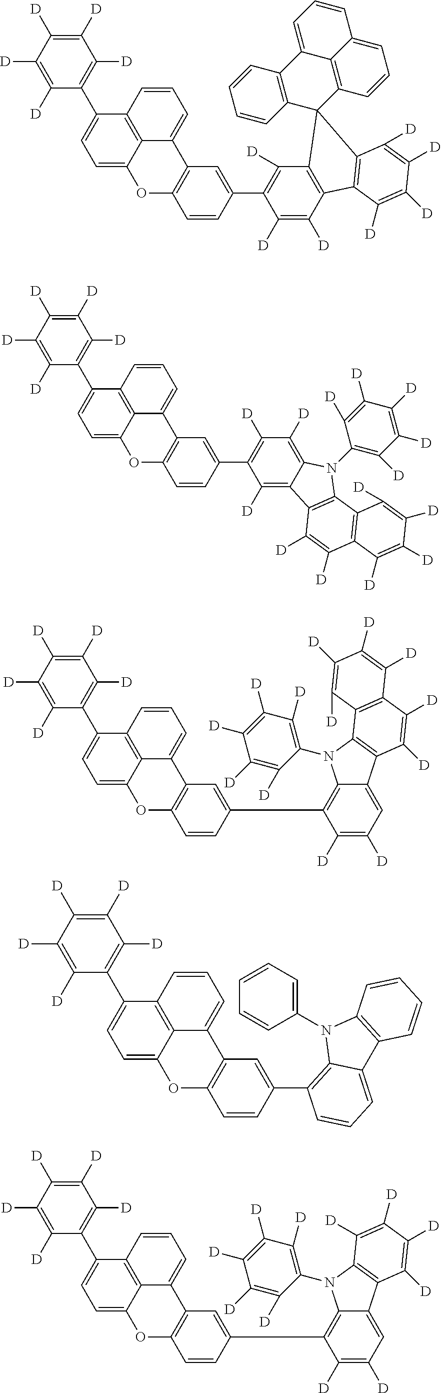

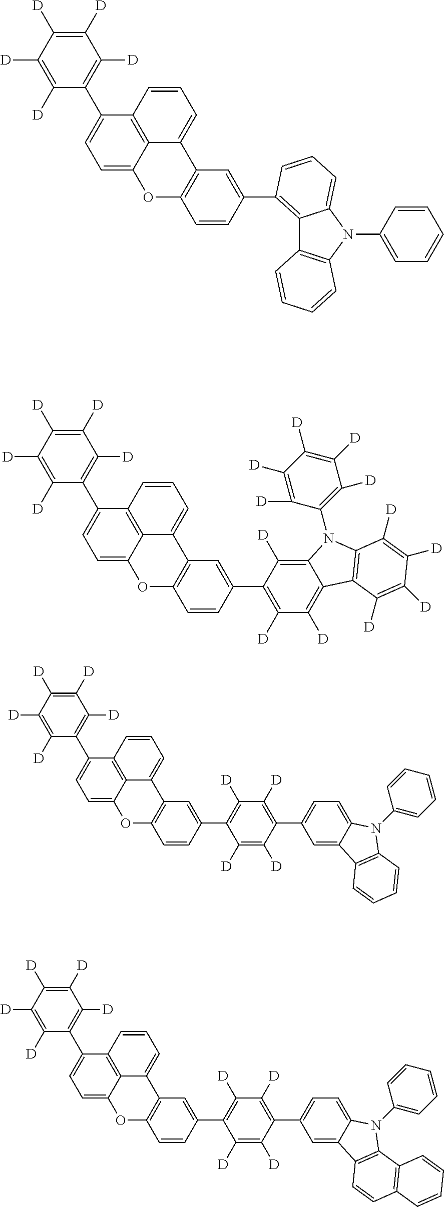

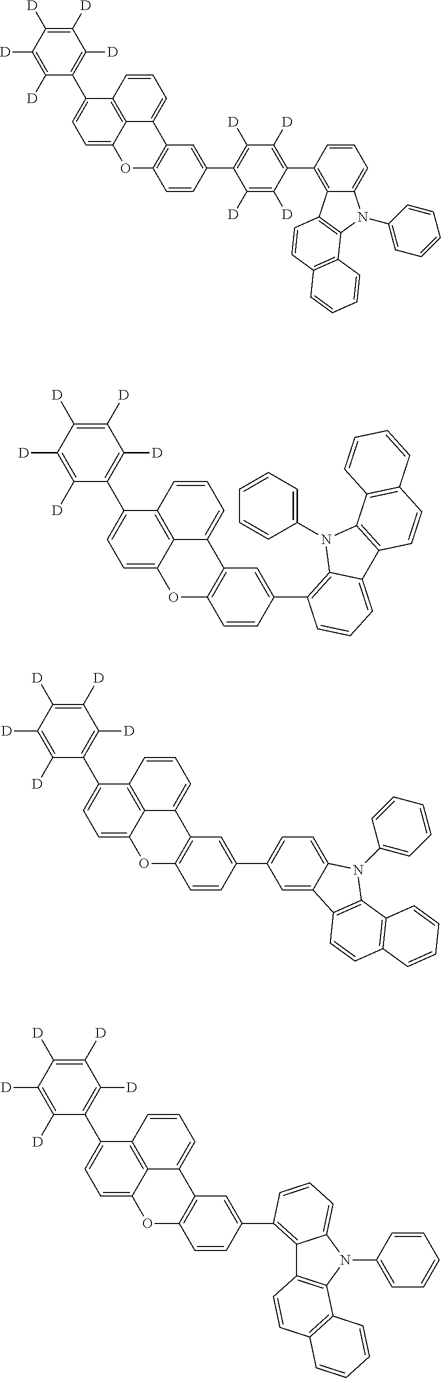 [SMILES: [2H]c1c([2H])c([2H])c(-c2ccc3c4c(cccc24)-c2cc(-c4c([2H])c([2H])c(-c5cccc6c5c5ccc7ccccc7c5n6-c5ccccc5)c([2H])c4[2H])ccc2O3)c([2H])c1[2H] ; [2H]c1c([2H])c([2H])c(-c2ccc3c4c(cccc24)-c2cc(-c4cccc5c6ccc7ccccc7c6n(-c6ccccc6)c45)ccc2O3)c([2H])c1[2H] ; [2H]c1c([2H])c([2H])c(-c2ccc3c4c(cccc24)-c2cc(-c4ccc5c(c4)c4ccc6ccccc6c4n5-c4ccccc4)ccc2O3)c([2H])c1[2H] ; [2H]c1c([2H])c([2H])c(-c2ccc3c4c(cccc24)-c2cc(-c4cccc5c4c4ccc6ccccc6c4n5-c4ccccc4)ccc2O3)c([2H])c1[2H]]

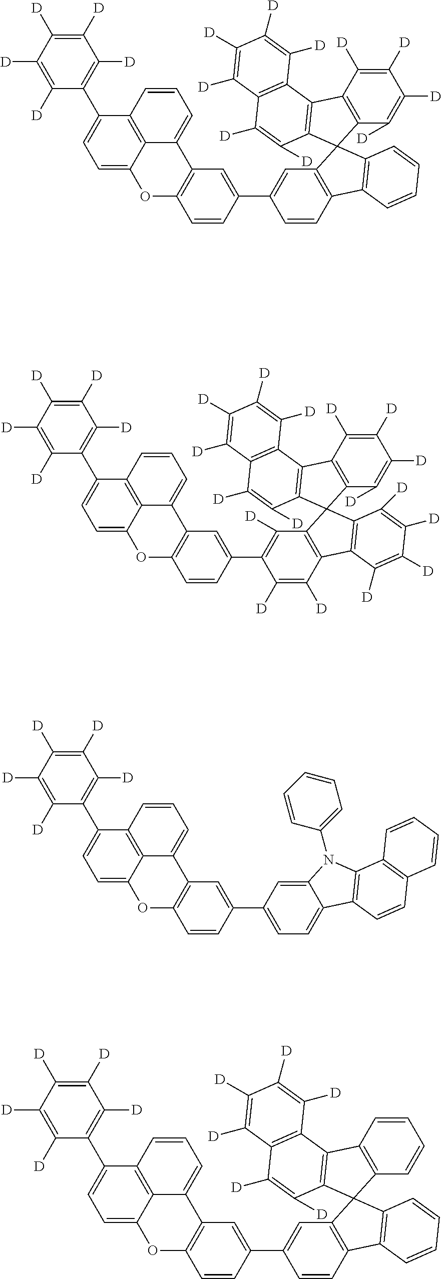

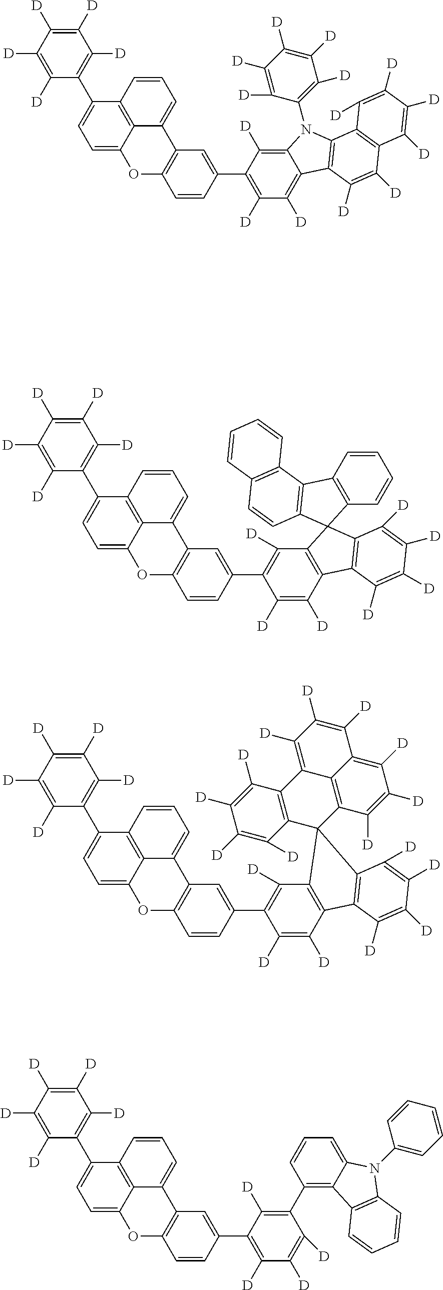

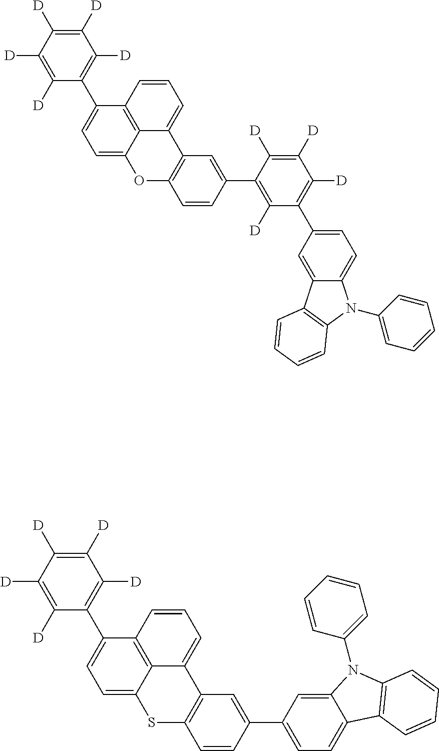 [SMILES: [2H]c1c([2H])c([2H])c(-c2ccc3c4c(cccc24)-c2cc(-c4c([2H])c([2H])c([2H])c(-c5ccc6c(c5)c5ccccc5n6-c5ccccc5)c4[2H])ccc2O3)c([2H])c1[2H] ; [2H]c1c([2H])c([2H])c(-c2ccc3c4c(cccc24)-c2cc(-c4ccc5c6ccccc6n(-c6ccccc6)c5c4)ccc2S3)c([2H])c1[2H]]

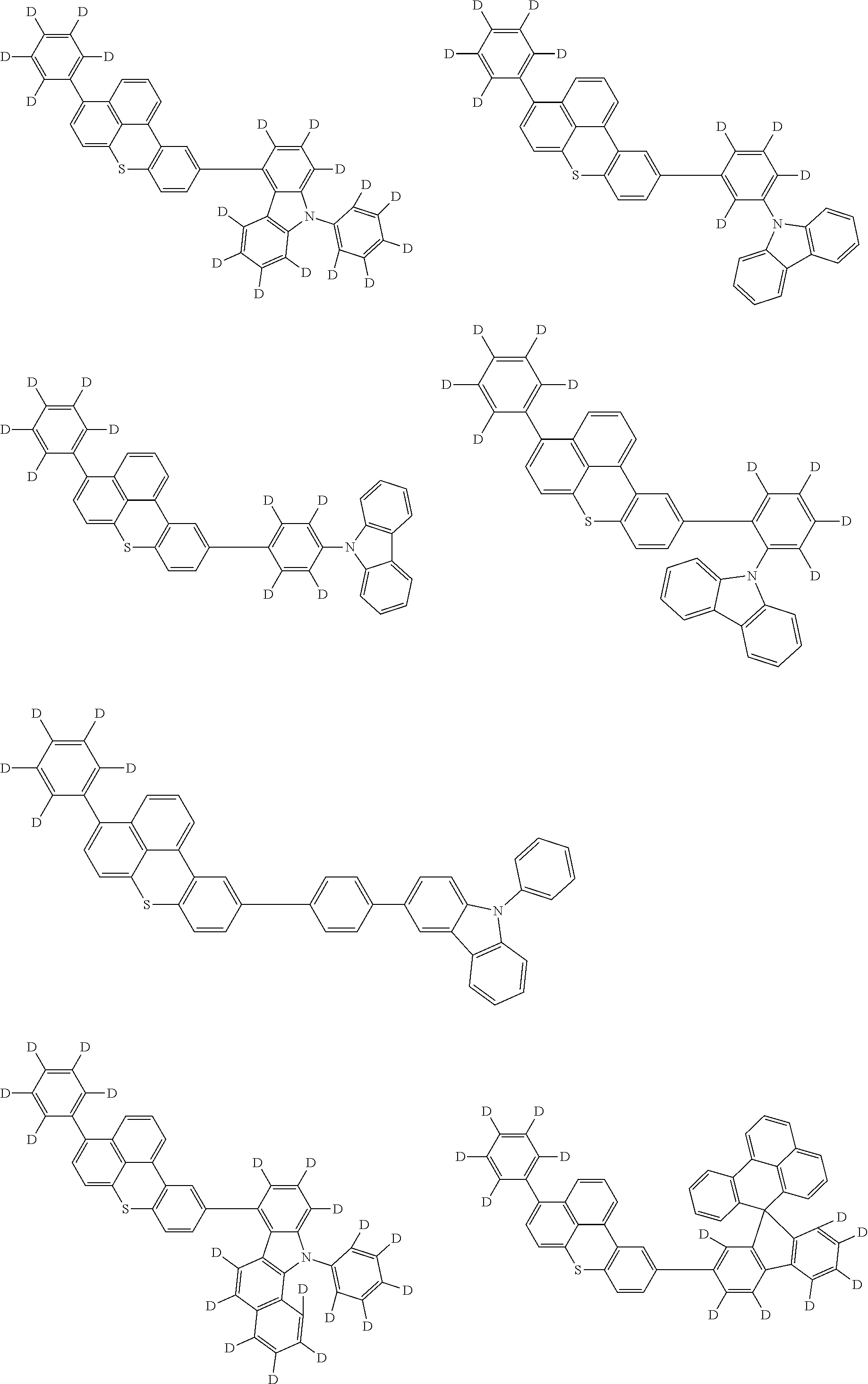

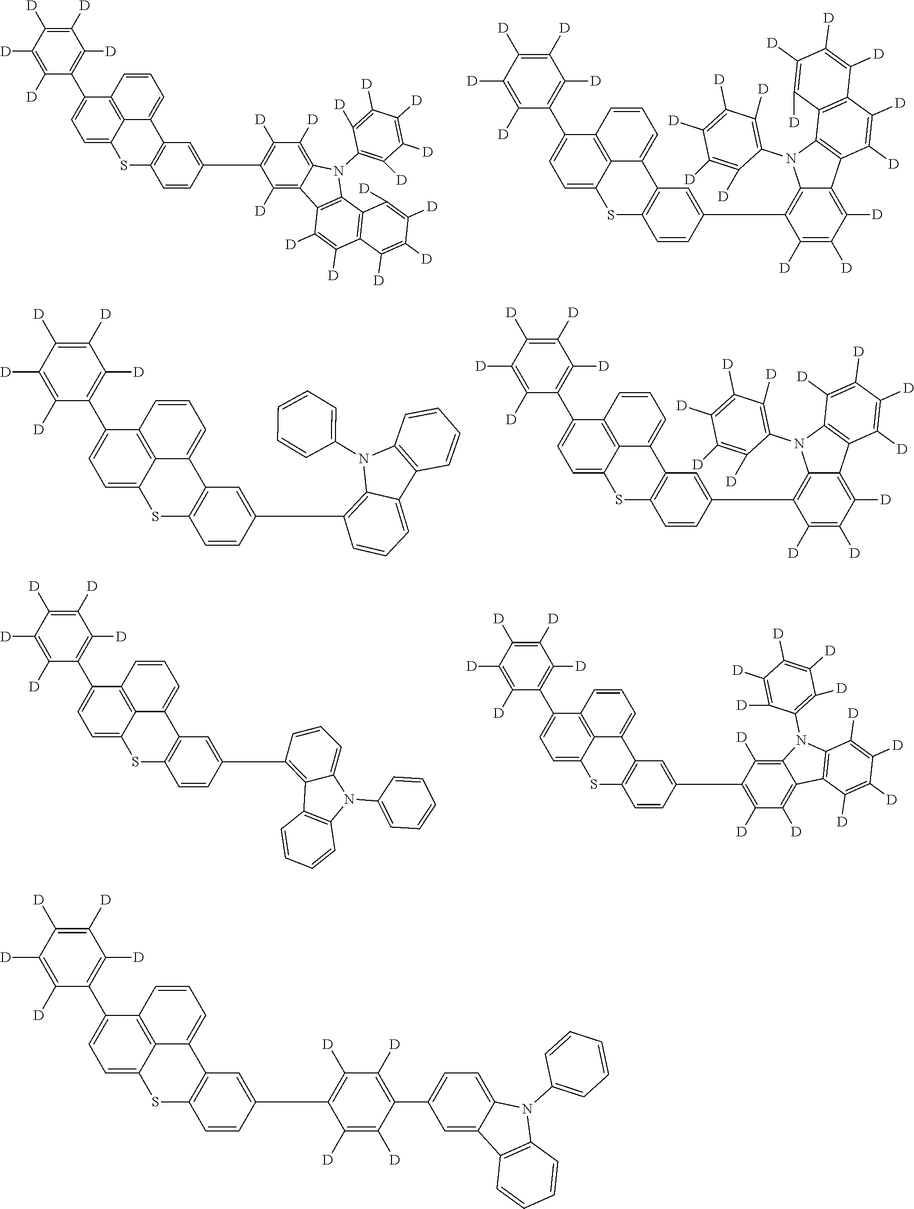

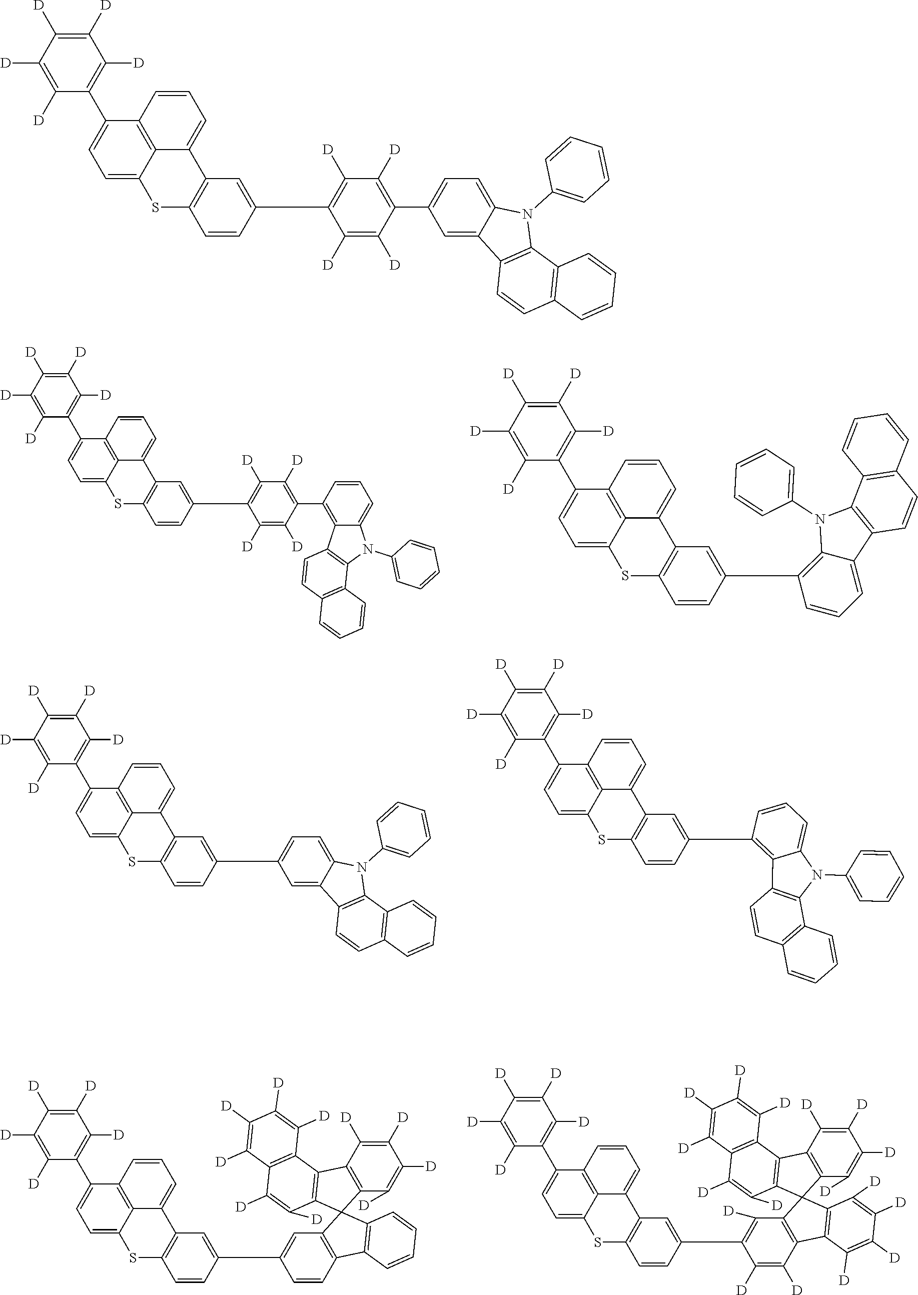

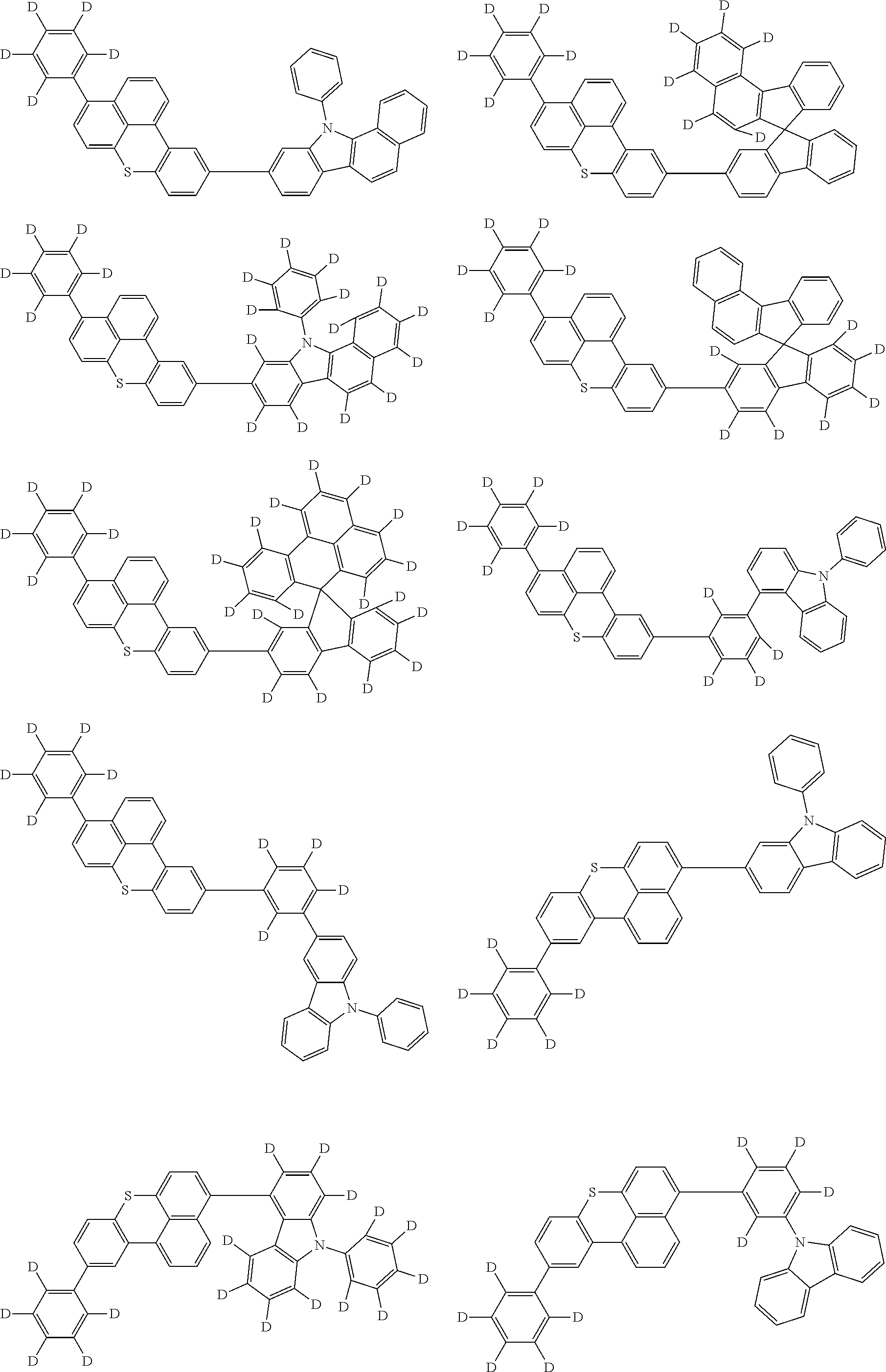

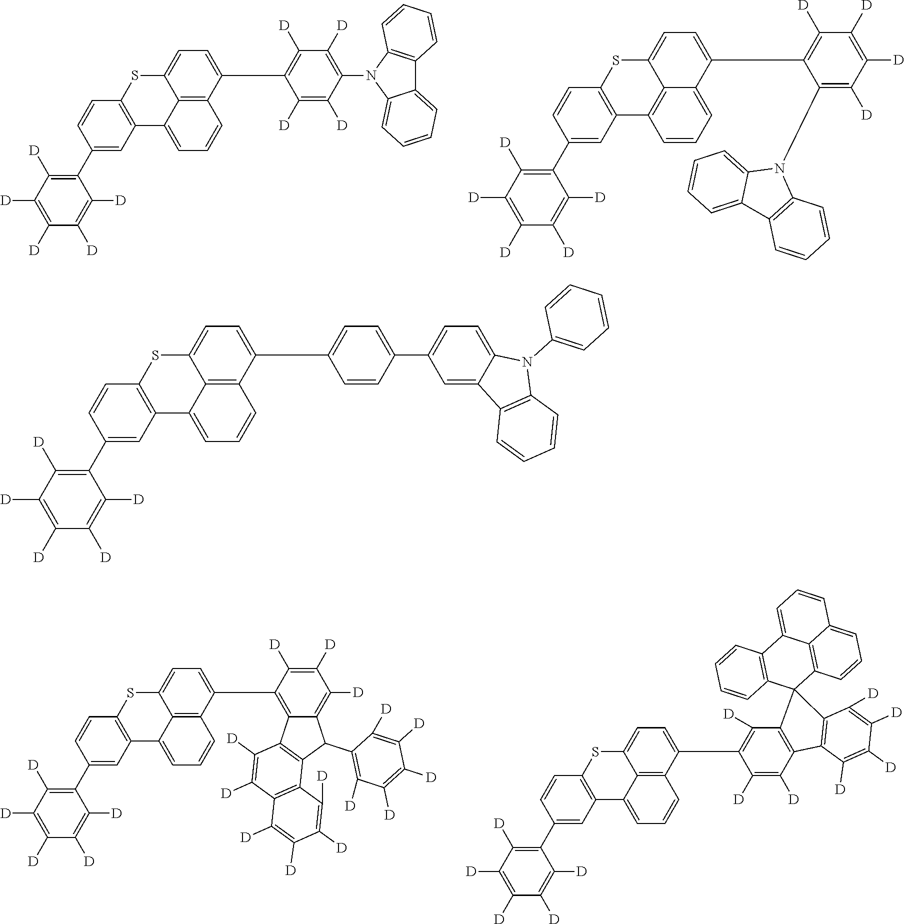

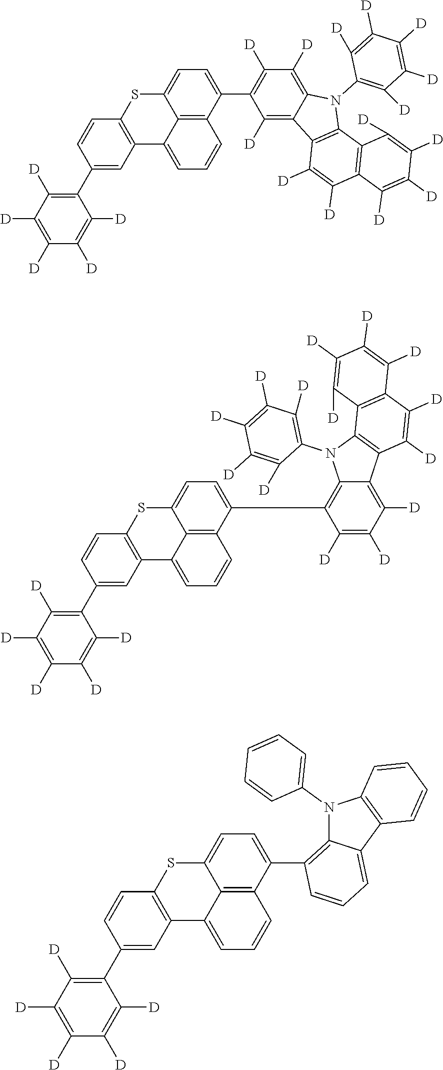 [SMILES: [2H]c1c([2H])c([2H])c(-c2ccc3c(c2)-c2cccc4c(-c5c([2H])c([2H])c6c(c5[2H])c5c([2H])c([2H])c7c([2H])c([2H])c([2H])c([2H])c7c5n6-c5c([2H])c([2H])c([2H])c([2H])c5[2H])ccc(c24)S3)c([2H])c1[2H] ; [2H]c1c([2H])c([2H])c(-c2ccc3c(c2)-c2cccc4c(-c5c([2H])c([2H])c([2H])c6c7c([2H])c([2H])c8c([2H])c([2H])c([2H])c([2H])c8c7n(-c7c([2H])c([2H])c([2H])c([2H])c7[2H])c56)ccc(c24)S3)c([2H])c1[2H] ; [2H]c1c([2H])c([2H])c(-c2ccc3c(c2)-c2cccc4c(-c5cccc6c7ccccc7n(-c7ccccc7)c56)ccc(c24)S3)c([2H])c1[2H]]

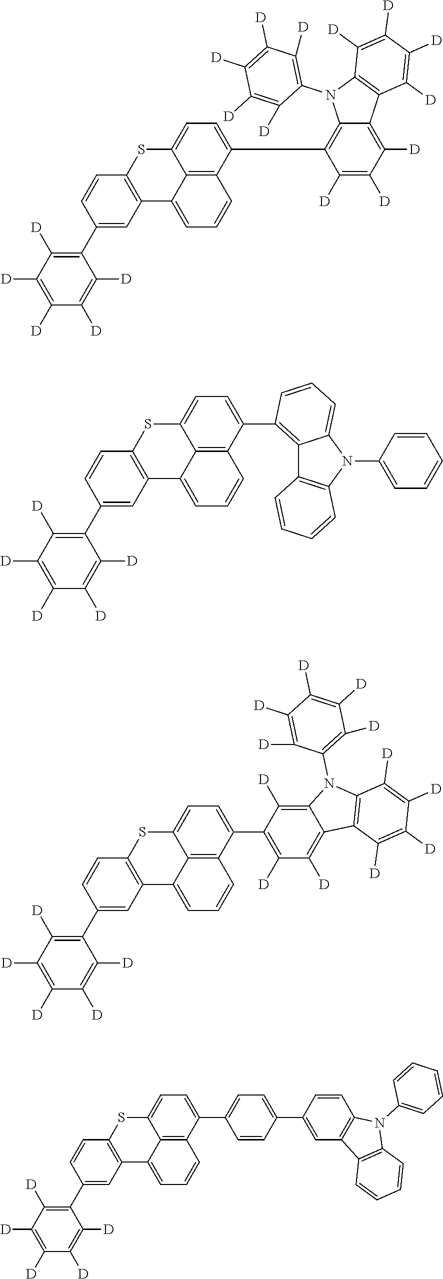

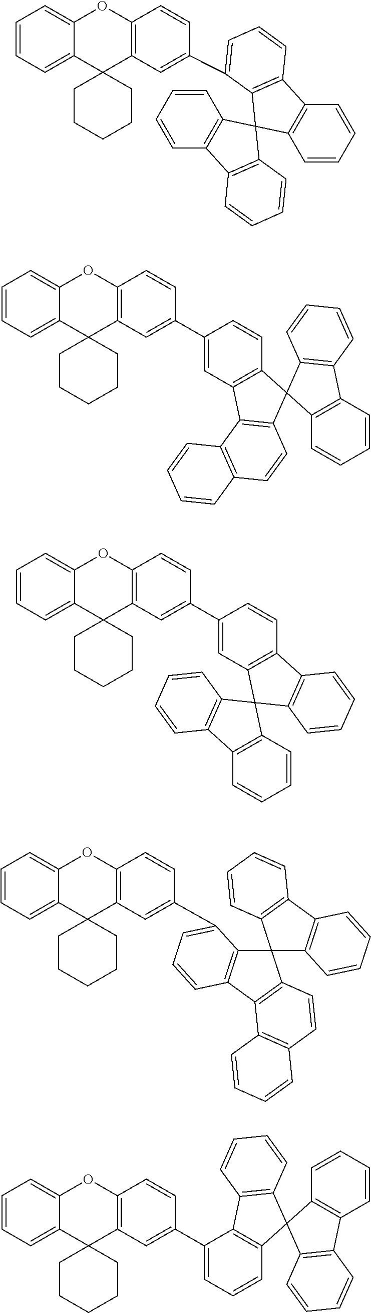

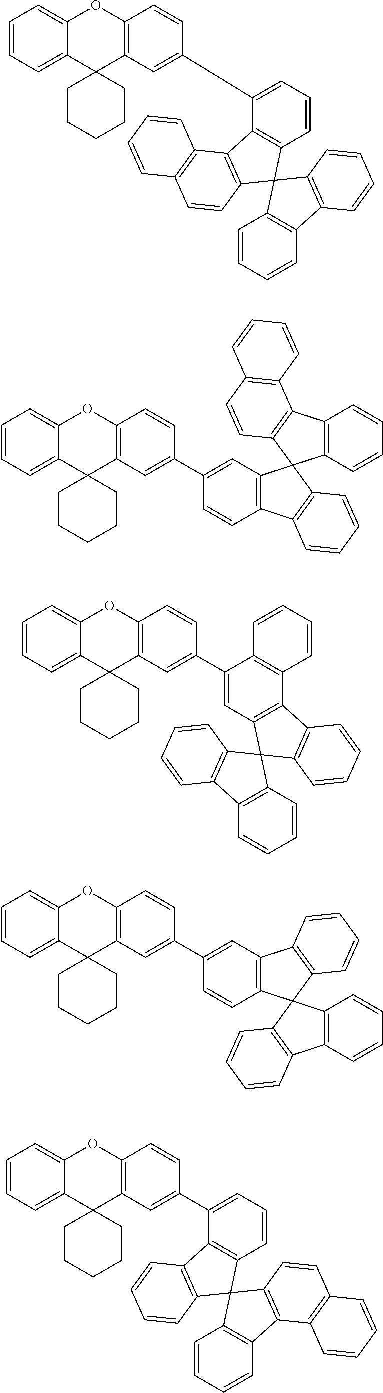

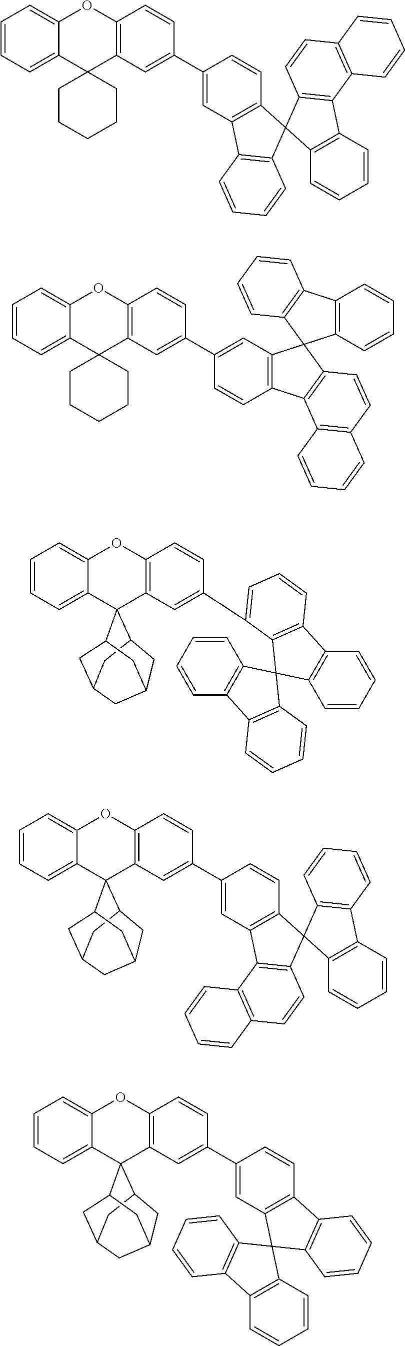

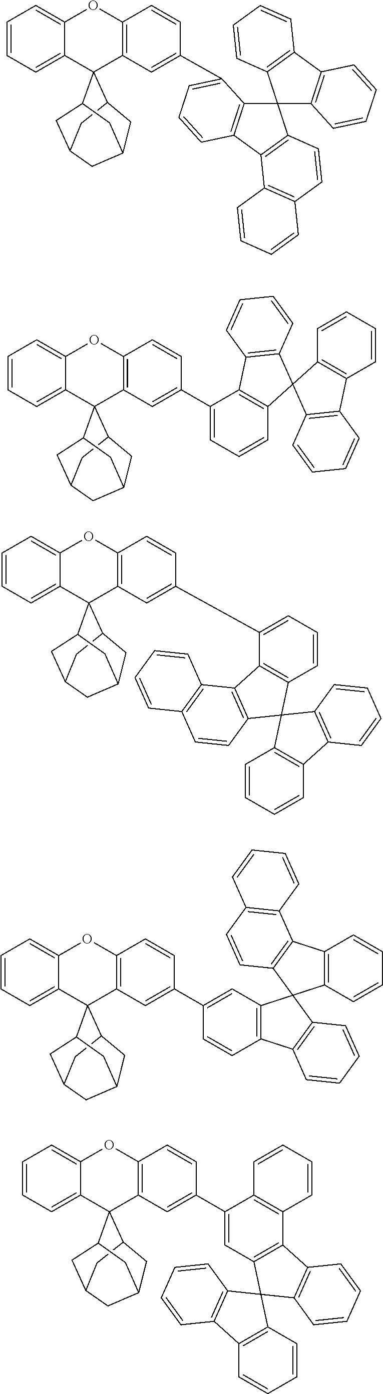

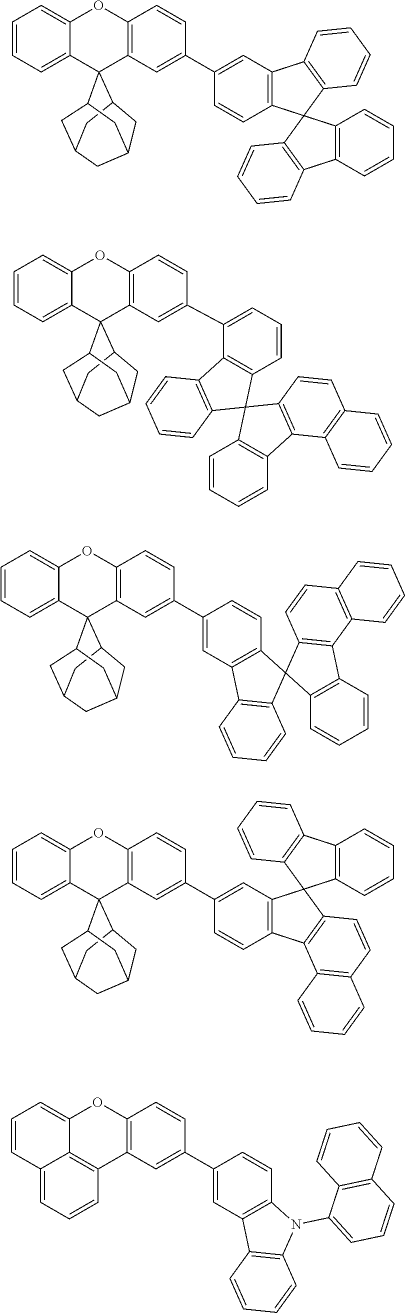

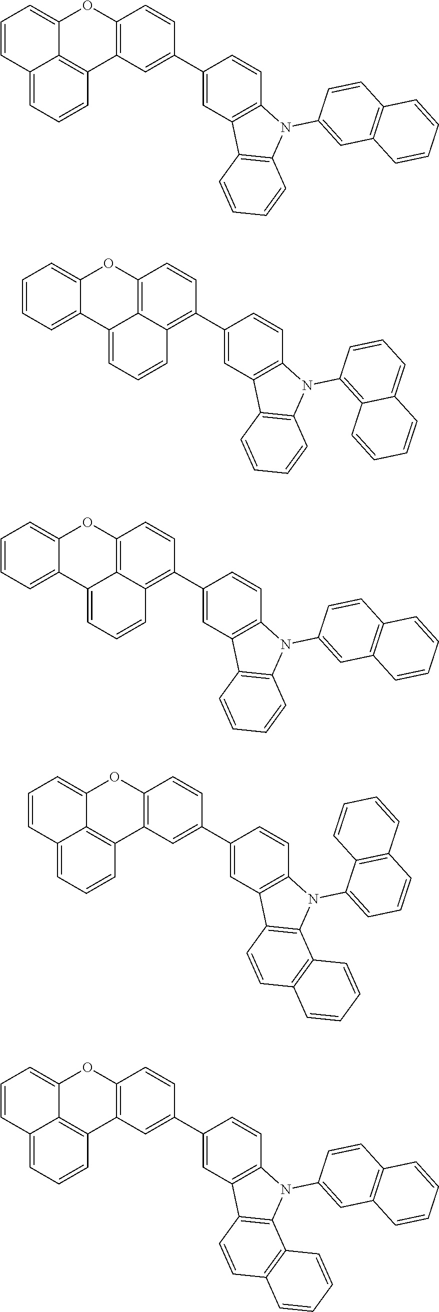 [SMILES: c1ccc2cc(-n3c4ccccc4c4cc(-c5ccc6c(c5)-c5cccc7cccc(c57)O6)ccc43)ccc2c1 ; c1ccc2c(c1)Oc1ccc(-c3ccc4c(c3)c3ccccc3n4-c3cccc4ccccc34)c3cccc-2c13 ; c1ccc2c(c1)Oc1ccc(-c3ccc4c(c3)c3ccccc3n4-c3ccc4ccccc4c3)c3cccc-2c13 ; c1ccc2c(-n3c4ccc(-c5ccc6c(c5)-c5cccc7cccc(c57)O6)cc4c4ccc5ccccc5c43)cccc2c1 ; c1ccc2cc(-n3c4ccc(-c5ccc6c(c5)-c5cccc7cccc(c57)O6)cc4c4ccc5ccccc5c43)ccc2c1]

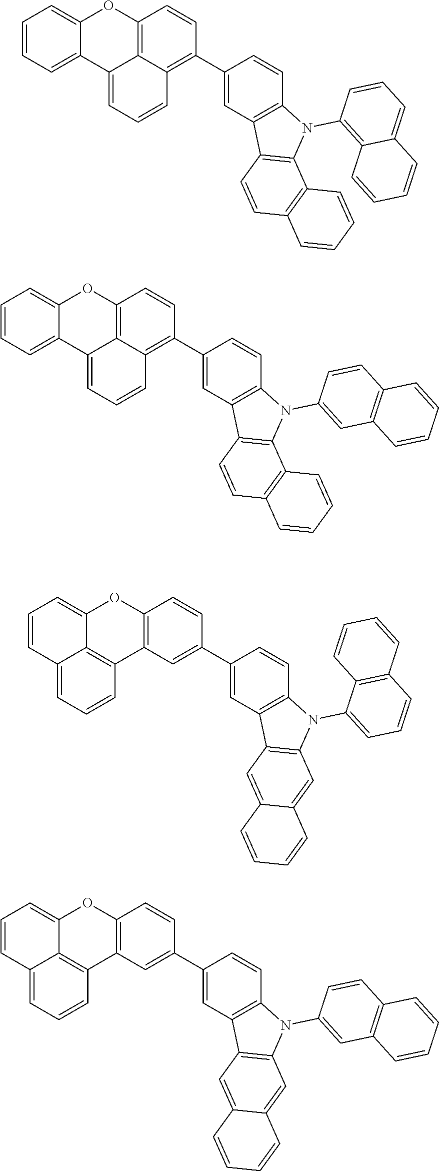 [SMILES: c1ccc2c(c1)Oc1ccc(-c3ccc4c(c3)c3ccc5ccccc5c3n4-c3cccc4ccccc34)c3cccc-2c13 ; c1ccc2c(c1)Oc1ccc(-c3ccc4c(c3)c3ccc5ccccc5c3n4-c3ccc4ccccc4c3)c3cccc-2c13 ; c1ccc2cc3c(cc2c1)c1cc(-c2ccc4c(c2)-c2cccc5cccc(c25)O4)ccc1n3-c1cccc2ccccc12 ; c1ccc2cc(-n3c4ccc(-c5ccc6c(c5)-c5cccc7cccc(c57)O6)cc4c4cc5ccccc5cc43)ccc2c1]

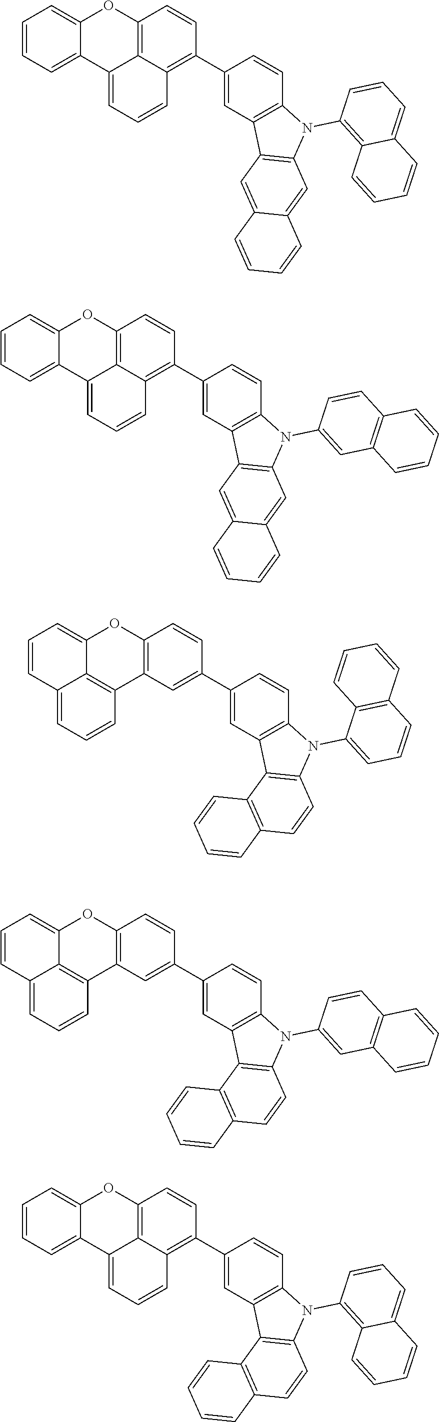

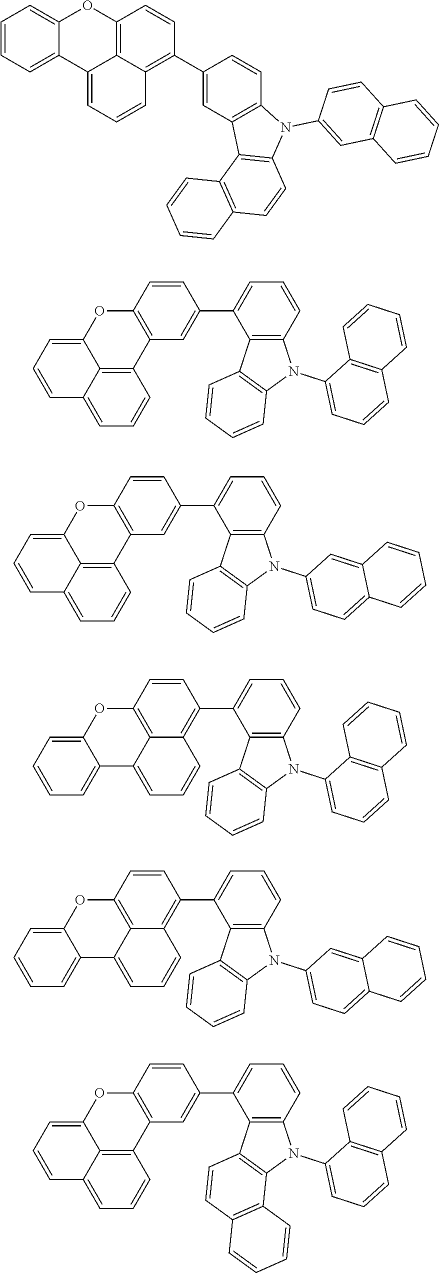

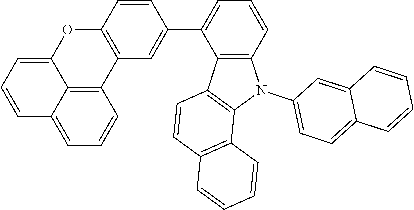 [SMILES: c1ccc2cc(-n3c4cccc(-c5ccc6c(c5)-c5cccc7cccc(c57)O6)c4c4ccc5ccccc5c43)ccc2c1]

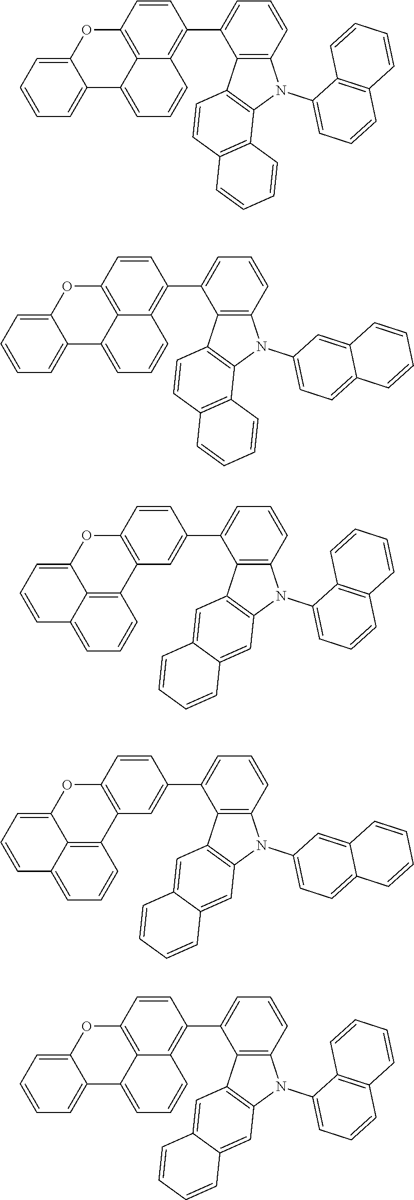

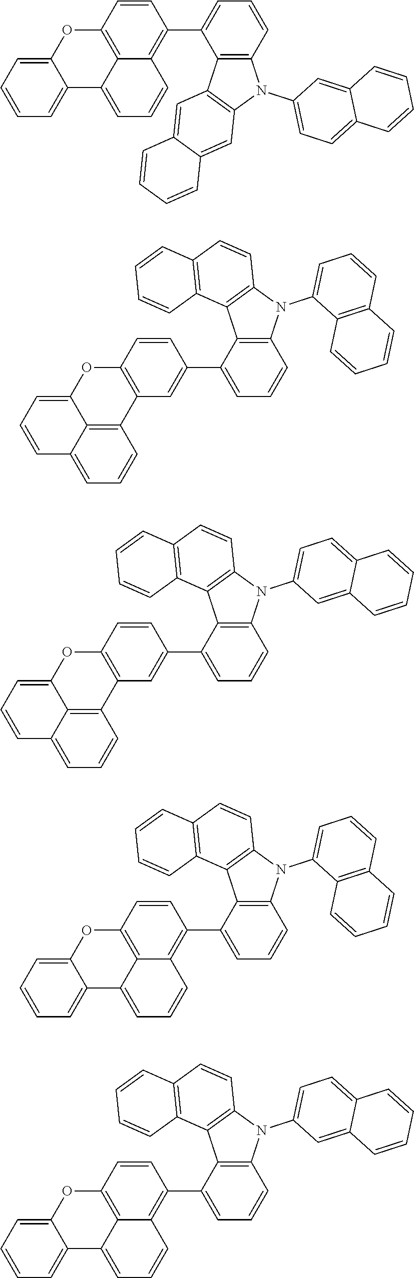

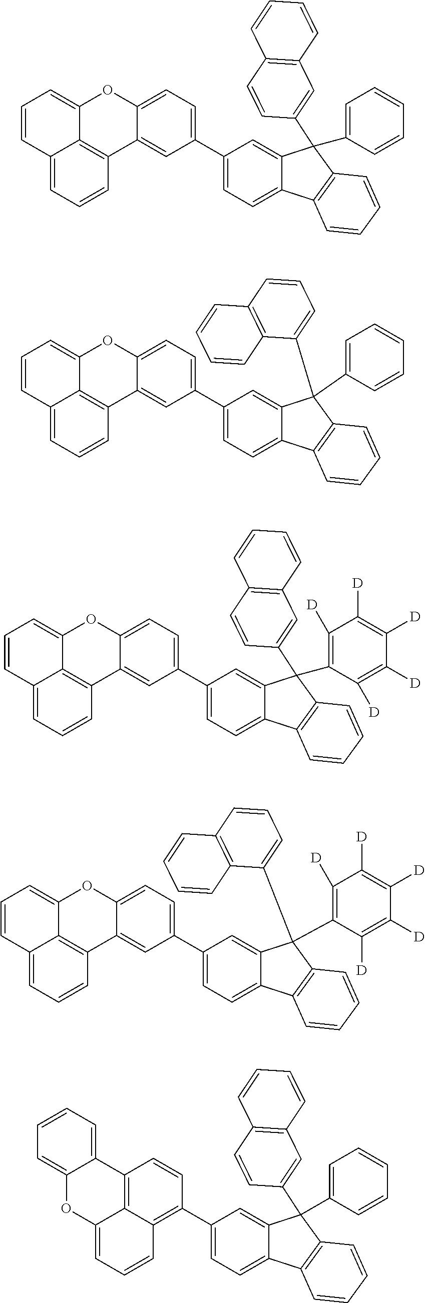 [SMILES: c1ccc(C2(c3ccc4ccccc4c3)c3ccccc3-c3ccc(-c4ccc5c(c4)-c4cccc6cccc(c46)O5)cc32)cc1 ; c1ccc(C2(c3cccc4ccccc34)c3ccccc3-c3ccc(-c4ccc5c(c4)-c4cccc6cccc(c46)O5)cc32)cc1 ; [2H]c1c([2H])c([2H])c(C2(c3ccc4ccccc4c3)c3ccccc3-c3ccc(-c4ccc5c(c4)-c4cccc6cccc(c46)O5)cc32)c([2H])c1[2H] ; [2H]c1c([2H])c([2H])c(C2(c3cccc4ccccc34)c3ccccc3-c3ccc(-c4ccc5c(c4)-c4cccc6cccc(c46)O5)cc32)c([2H])c1[2H] ; c1ccc(C2(c3ccc4ccccc4c3)c3ccccc3-c3ccc(-c4ccc5c6c(cccc46)Oc4ccccc4-5)cc32)cc1]

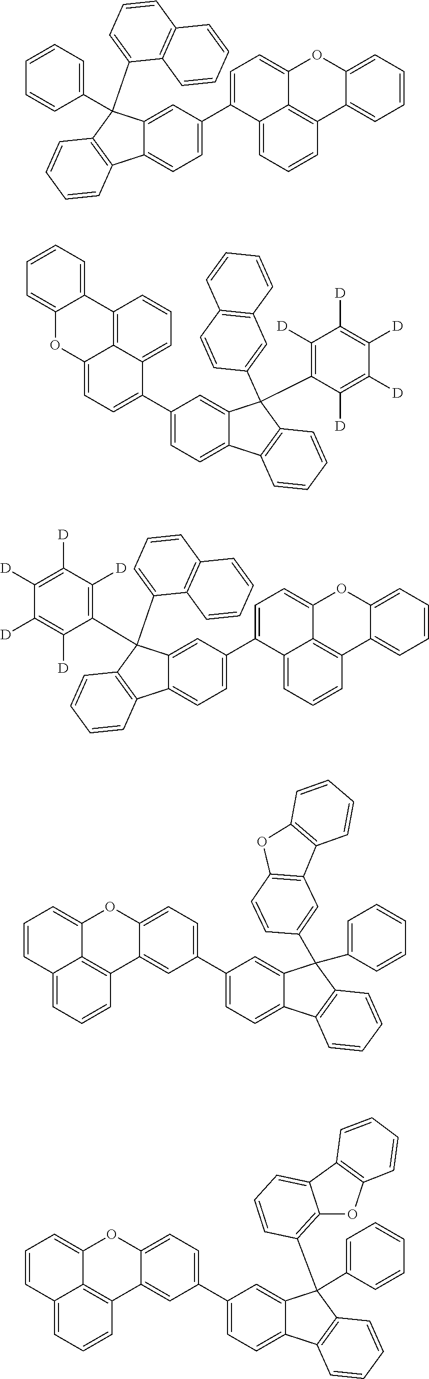 [SMILES: c1ccc(C2(c3cccc4ccccc34)c3ccccc3-c3ccc(-c4ccc5c6c(cccc46)-c4ccccc4O5)cc32)cc1 ; [2H]c1c([2H])c([2H])c(C2(c3ccc4ccccc4c3)c3ccccc3-c3ccc(-c4ccc5c6c(cccc46)-c4ccccc4O5)cc32)c([2H])c1[2H] ; [2H]c1c([2H])c([2H])c(C2(c3cccc4ccccc34)c3ccccc3-c3ccc(-c4ccc5c6c(cccc46)-c4ccccc4O5)cc32)c([2H])c1[2H] ; c1ccc(C2(c3ccc4oc5ccccc5c4c3)c3ccccc3-c3ccc(-c4ccc5c(c4)-c4cccc6cccc(c46)O5)cc32)cc1 ; c1ccc(C2(c3cccc4c3oc3ccccc34)c3ccccc3-c3ccc(-c4ccc5c(c4)-c4cccc6cccc(c46)O5)cc32)cc1]

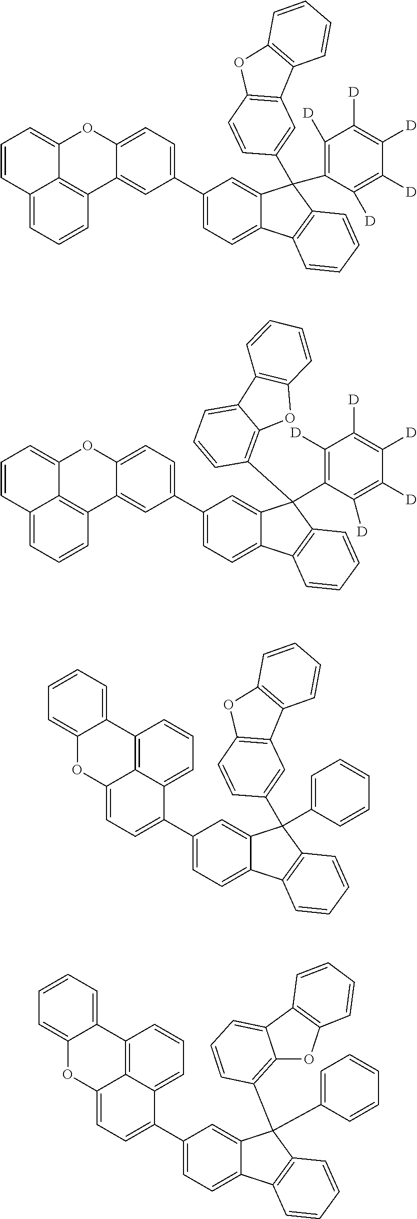 [SMILES: [2H]c1c([2H])c([2H])c(C2(c3ccc4oc5ccccc5c4c3)c3ccccc3-c3ccc(-c4ccc5c(c4)-c4cccc6cccc(c46)O5)cc32)c([2H])c1[2H] ; [2H]c1c([2H])c([2H])c(C2(c3cccc4c3oc3ccccc34)c3ccccc3-c3ccc(-c4ccc5c(c4)-c4cccc6cccc(c46)O5)cc32)c([2H])c1[2H] ; c1ccc(C2(c3ccc4oc5ccccc5c4c3)c3ccccc3-c3ccc(-c4ccc5c6c(cccc46)-c4ccccc4O5)cc32)cc1 ; c1ccc(C2(c3cccc4c3oc3ccccc34)c3ccccc3-c3ccc(-c4ccc5c6c(cccc46)-c4ccccc4O5)cc32)cc1]

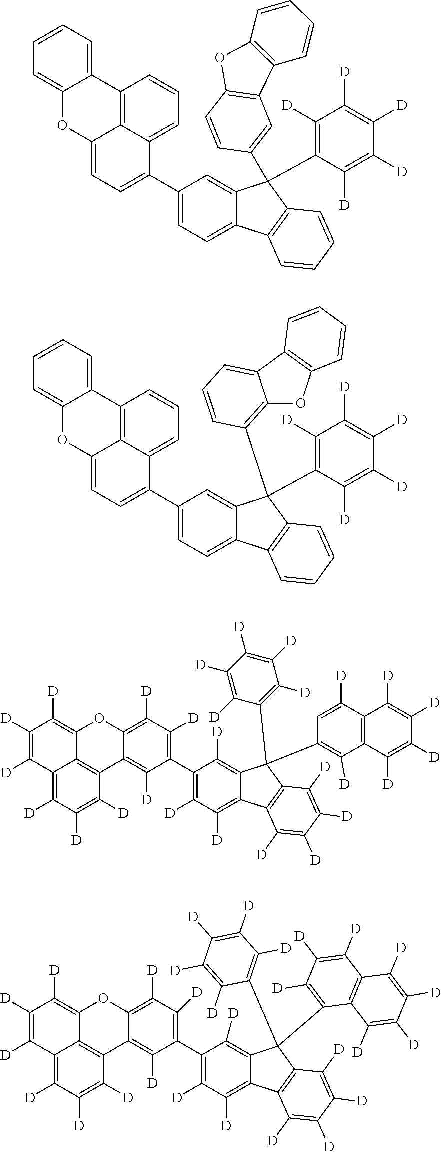

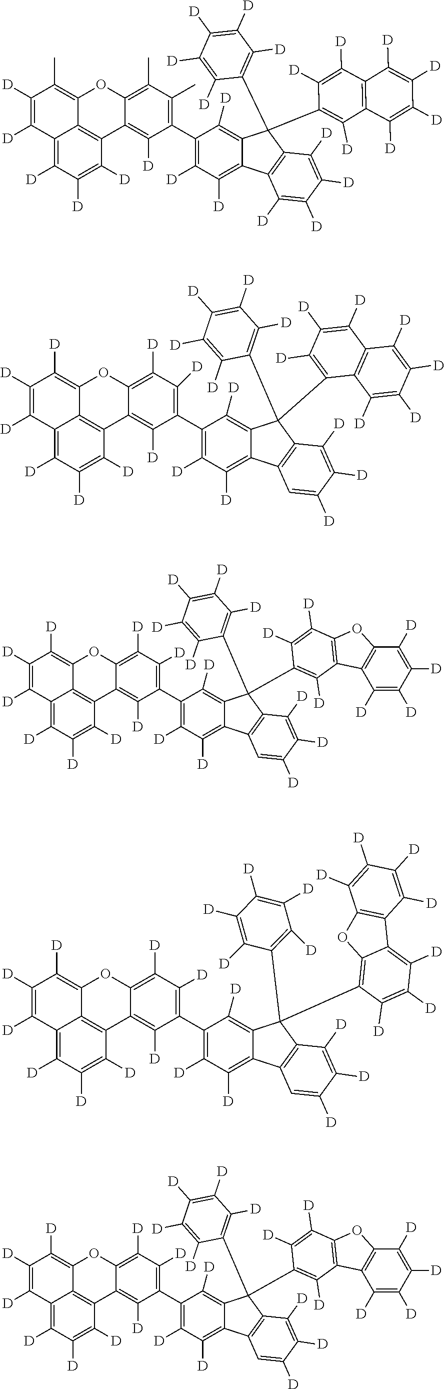

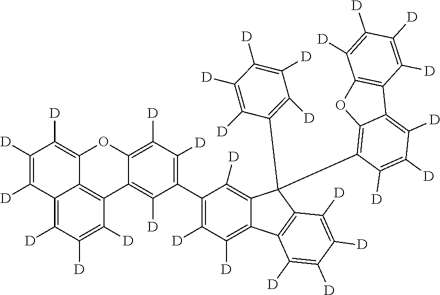 [SMILES: [2H]c1c([2H])c([2H])c(C2(c3c([2H])c([2H])c([2H])c4c3oc3c([2H])c([2H])c([2H])c([2H])c34)c3c([2H])c([2H])c([2H])c([2H])c3-c3c([2H])c([2H])c(-c4c([2H])c([2H])c5c(c4[2H])-c4c([2H])c([2H])c([2H])c6c([2H])c([2H])c([2H])c(c46)O5)c([2H])c32)c([2H])c1[2H]]

### [Material for Organic EL Devices]

The material for organic EL devices of one embodiment of the present invention contains the inventive compound. The content of the inventive compound in the material for organic EL devices is 1% by mass or more (including 100%), preferably 10% by mass or more (including 100%), more preferably 50% by mass or more (including 100%), still more preferably 80% by mass or more (including 100%), and particularly preferably 90% by mass or more (including 100%). The material for organic EL devices of one embodiment of the present invention is useful for the production of an organic EL device.

In one embodiment of the present invention, the inventive compound is preferably a hole transporting layer material.

In another embodiment of the present invention, the inventive compound is preferably a host material used in a light emitting layer.

In one embodiment of the present invention, the material for organic EL devices preferably further contains a protium compound of the inventive compound. The protium compound is a compound in which all hydrogen atoms in the inventive compound are protium atoms.

The mixing molar ratio of the inventive compound to the protium compound of the inventive compound (inventive compound: protium compound) is preferably 10:90 to 90:10, more preferably 20:80 to 80:20, still more preferably 30:70 to 70:30, and particularly preferably 40:60 to 60:40.

The material for organic electroluminescent devices according to one embodiment of the present invention is a hole transporting layer material.

The material for organic electroluminescent devices according to another embodiment of the present invention is a host material used in a light emitting layer.

The content of the inventive compound in the material for organic electroluminescent devices is preferably 1% by mass or more (including 100%), more preferably 10% by mass or more (including 100%), still more preferably 50% by mass or more (including 100%), even more preferably 80% by mass or more (including 100%), and particularly preferably 90% by mass or more (including 100%).

### [Organic EL Device]

### <First Organic EL Device>

A first organic EL device according to an embodiment of the present invention includes an anode, a cathode, and one or more organic layers intervening between the anode and the cathode. The organic layers include a light emitting layer, and at least one layer of the organic layers contains the inventive compound.

Examples of the organic layer containing the inventive compound include a hole transporting zone (such as a hole injecting layer, a hole transporting layer, an electron blocking layer, and an exciton blocking layer) intervening between the anode and the light emitting layer, the light emitting layer, a space layer, and an electron transporting zone (such as an electron injecting layer, an electron transporting layer, and a hole blocking layer) intervening between the cathode and the light emitting layer, but are not limited thereto. The inventive compound is preferably used as a material for the hole transporting zone or the light emitting layer in a fluorescent or phosphorescent EL device, more preferably as a material for the hole transporting zone or a host material used for the light emitting layer, still more preferably as a material for the hole injecting layer, the hole transporting layer, the electron blocking layer, or the exciton blocking layer, or as a host material, particularly preferably as a material for the hole injecting layer or the hole transport layer or as a host material, and most preferably as a material for the hole injecting layer or the hole transporting layer.

### <Second Organic EL Device>

A second organic EL device according to an embodiment of the present invention includes an anode, a hole transporting zone, a light emitting layer, and a cathode in this order, and the hole transporting zone contains a compound A satisfying the following conditions (A) to (C):
(A) a highest occupied molecular orbital energy level HOMO is −6.00 to −5.50 eV;
(B) a triplet energy T₁ is 2.10 eV or more; and
(C) an 80% attenuation time t of photoluminescence intensity PL is 0.10 h or more,

With respect to the above condition (A), in the description herein, the highest occupied molecular orbital energy level HOMO of the compound A is measured by using a photoelectron spectrometer under the atmosphere. Specifically, the highest occupied molecular orbital energy level HOMO of the compound A can be measured by a method described in Examples.

With respect to the condition (B), the triplet energy T₁ can be measured by the following method.

A compound to be measured is dissolved in EPA (diethyl ether:isopentane:ethanol=5:5:2 (volume ratio)) at a concentration of 10⁻⁵ mol/L or more and 10⁻⁴ mol/L or less to prepare a solution, and the solution is placed in a quartz cell to be used as a measurement sample. For this measurement sample, a phosphorescence spectrum (vertical axis: phosphorescence emission intensity, horizontal axis: wavelengths) is measured at a low temperature (77 [K]), a tangent line is drawn to the rise on the short wavelength side of this phosphorescence spectrum, and the energy amount calculated from the following conversion formula (F1) based on the wavelengths λ_{edge} [nm] at the intersection of the tangent line and the horizontal axis is taken as the triplet energy T₁.

T₁ [eV]=1239.85/λ_{edge} Conversion formula (F1):

The tangent line to the rise on the short wavelength side of the phosphorescence spectrum is drawn as follows. When moving on the spectrum curve from the short wavelength side of the phosphorescence spectrum to the maximum value on the shortest wavelength side among the maximum values of the spectrum, the tangent line at each point on the curve is considered toward the long wavelength side. This tangent line increases in slope as the curve rises (i.e., as the vertical axis increases). A tangent line drawn at a point at which the slope has a maximum value (i.e., a tangent line at the inflection point) is regarded as a tangent line to the rise of the phosphorescence spectrum on the short wavelength side.

Note that a maximum point having a peak intensity of 15% or less of the maximum peak intensity of the spectrum is not included in the above-described maximum value on the shortest wavelength side, and a tangent line drawn at a point which is closest to the maximum value on the shortest wavelength side and at which the value of the slope is a maximum value is regarded as a tangent line to the rise on the short wavelength side of the phosphorescence spectrum.

For the measurement of phosphorescence, an F-4500 type fluorescence spectrophotometer main body manufactured by Hitachi High-Tech Corporation can be used. The measuring device is not limited thereto, and the measurement may be performed by combining a cooling device, a low-temperature vessel, an exciting light source, and a light receiving device.

In the above condition (C), the photoluminescence intensity PL is an intensity of a photoluminescence emission spectrum when a measurement material in which a compound to be measured is formed into a film having a film thickness of 100 nm is irradiated with ultraviolet rays of 365 nm at an irradiation intensity I₁.

In the condition (C), the 80% attenuation time t of the photoluminescence intensity PL is a time from the start of the irradiation with the ultraviolet rays to the attenuation of the photoluminescence intensity PL to 80%.

The irradiation intensity I₁ is defined by the following mathematical expression (Equation 1).

I₁=I₀×(A₀/A₁) (Equation 1)

In the mathematical expression (Equation 1), I₀ is an irradiation intensity at the time of PL measurement of a reference material in which a compound represented by the following chemical formula is formed into a film having a film thickness of 100 nm.

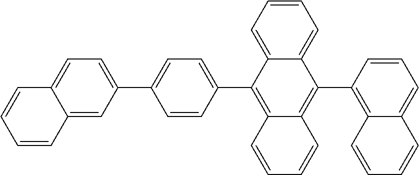 [SMILES: c1ccc2cc(-c3ccc(-c4c5ccccc5c(-c5cccc6ccccc56)c5ccccc45)cc3)ccc2c1]

In the above mathematical expression (Equation 1), A₀ is an absorptance of the reference material, and A₁ is an absorptance of the measurement material. Each absorptance is defined by the following mathematical expression (Equation 2).

$$Absorptance = 1 - EXP ( - 4 \times 3.1416 \times ko \times d / w ) \qquad ( Equation 2 )$$

In the mathematical expression (Equation 2), ko is an extinction coefficient in an in-plane direction of a measurement material or a reference material on which a film of a compound to be measured is formed, d is a film thickness of the measurement material or the reference material on which a film of the compound to be measured is formed, and w is a wavelength of irradiation light.

Here, the extinction coefficient ko is measured by the following procedure. A material to be measured is vacuum-deposited on a glass substrate to a film thickness of about 50 nm to prepare a sample to be measured, and the sample is irradiated with incident light (ultraviolet to visible light to near-infrared light) every 5° in a measurement angle range of 45° to 75° with a spectroscopic ellipsometry device (M-2000UI, manufactured by J. A. Woollam Company, Inc., USA) to measure a change in a polarization state of light reflected from the sample surface. In order to increase the measurement accuracy of the extinction coefficient, the transmission spectrum in the substrate normal direction (direction perpendicular to the surface of the organic EL device substrate) is also measured by the device. Similarly, only the glass substrate on which the material to be measured is not vapor-deposited is subjected to the same measurement. The obtained measurement information is subjected to fitting with analysis software (Complete EASE) manufactured by J. A. Woollam Company, Inc.

As the fitting conditions, the refractive indices in the in-plane direction and the normal direction of the organic film formed on the substrate, the extinction coefficients in the in-plane direction and the normal direction, and the order parameter are calculated using a uniaxial rotationally symmetric anisotropic model such that the parameter MSE indicating the mean square error in the software is 3.0 or less. The order parameter is calculated from the peak wavelengths of the S1, with the peak on the long wavelength side of the extinction coefficient (in-plane direction) as the S1. As the fitting condition for the glass substrate, an isotropic model is used.

The film of the low-molecular-weight material vacuum-deposited on the substrate usually has uniaxial rotational symmetry with the substrate normal direction as the rotation target axis. In the case where an angle formed between a molecular axis in a thin film formed on a substrate and a substrate normal direction is denoted by θ, and extinction coefficients in a substrate parallel direction (Ordinary direction) and a substrate perpendicular direction (Extra-Ordinary direction) obtained by multiple incident angle spectroscopic ellipsometry measurement of the thin film are denoted by ko and ke, respectively, S′ represented by the following mathematical expressions (Equation 3) and (Equation 4) is an order parameter.

$${S}^{′} = 1 - 〈 cos 2 θ 〉 = 2 ko / ( ke + 2 ko ) = 2 / 3 ( 1 - S ) \qquad ( Equation 3 )$$

$$S = ( 1 / 2 ) 〈 3 cos 2 θ - 1 〉 = ( ke - ko ) / ( ke + 2 ko ) \qquad ( Equation 4 )$$

The method for evaluating the molecular orientation is a known technique, and details thereof are described in Organic Electronics, 2009, Vol. 10, p. 127. The method for forming the thin film is a vacuum deposition method.

The order parameter S′ obtained from the multiple incident angle spectroscopic ellipsometry measurement is 1.0 when all the molecules are oriented in the direction parallel to the substrate. The order parameter S′ is 0.66 when the molecules are not oriented and are random.

The organic EL device according to one embodiment of the present invention exhibits high device capability by having the above-described configuration. Specifically, it is possible to provide an organic EL device capable of achieving both high external quantum efficiency and a long life.

The organic EL device according to the embodiment of the present invention has the above-described characteristics, but is not limited thereto. When the compound A satisfying the above-described conditions (A) to (C) is contained in the hole transporting zone, the compound A has high excitation tolerance and has large triplet energy and an appropriate energy level of the highest occupied molecular orbital, and thus efficiency is easily increased. As a result, it is considered that the organic EL device can achieve both high external quantum efficiency and a long life.

### <Configuration of Organic EL Device>

A schematic configuration of an organic EL device according to one embodiment of the present invention will be described with reference to the drawings.

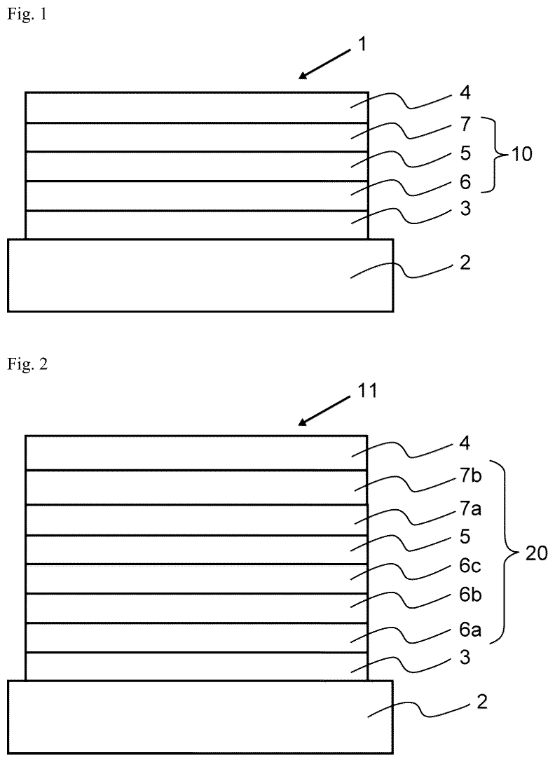

FIG. 1 is a schematic view showing an example of the configuration of the first and second organic EL devices. An organic EL device 1 shown in FIG. 1 includes a substrate 2, an anode 3, a cathode 4, and a light emitting unit 10 disposed between the anode 3 and the cathode 4. The light emitting unit 10 includes a light emitting layer 5. A hole transporting zone 6 (such as a hole injecting layer and a hole transporting layer) is provided between the light emitting layer 5 and the anode 3, and an electron transporting zone 7 (such as an electron injecting layer and an electron transporting layer) is provided between the light emitting layer 5 and the cathode 4. In addition, an electron blocking layer (which is not shown in the figure) may be provided on the side of the anode 3 of the light emitting layer 5, and a hole blocking layer (which is not shown in the figure) may be provided on the side of the cathode 4 of the light emitting layer 5. According to the configuration, electrons and holes are trapped in the light emitting layer 5, thereby enabling one to further increase the production efficiency of excitons in the light emitting layer 5.

FIG. 2 is a schematic view showing another configuration of the organic EL device. An organic EL device 11 shown in FIG. 2 includes a substrate 2, an anode 3, a cathode 4, and a light emitting unit 20 disposed between the anode 3 and the cathode 4. The light emitting unit 20 includes a light emitting layer 5. A hole transporting zone disposed between the anode 3 and the light emitting layer 5 is formed of a first hole transporting layer 6a and a second hole transporting layer 6b. In addition, the electron transporting zone disposed between the light emitting layer 5 and the cathode 4 is formed of a first electron transporting layer 7a and a second electron transporting layer 7b.

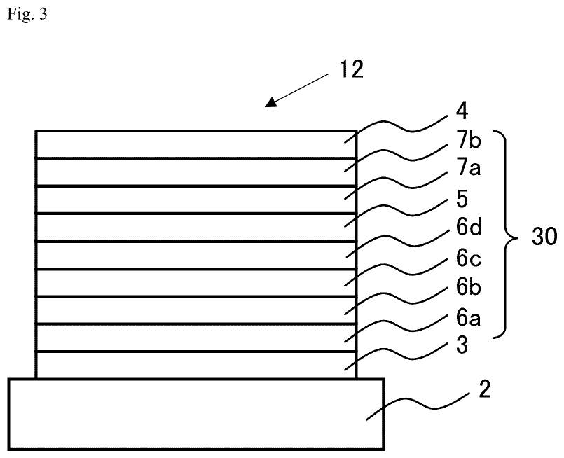

FIG. 3 is a schematic view showing still another configuration of the organic EL device according to one embodiment of the present invention. An organic EL device 12 includes a substrate 2, an anode 3, a cathode 4, and a light emitting unit 30 disposed between the anode 3 and the cathode 4. The light emitting unit 30 includes a light emitting layer 5. A hole transporting zone disposed between the anode 3 and the light emitting layer 5 is formed of a hole injecting layer 6a, a first hole transporting layer 6b, a second hole transporting layer 6c, and a third hole transporting layer 6d. In addition, the electron transporting zone disposed between the light emitting layer 5 and the cathode 4 is formed of a first electron transporting layer 7a and a second electron transporting layer 7b.

In FIGS. 1 to 3, the light emitting layer 5 includes at least one light emitting layer. The light emitting layer 5 may be a single layer or may include a plurality of layers (for example, a plurality of light emitting layers or a plurality of light emitting layers and a space layer).

In the description herein, the light emitting unit 10 in the organic EL device 1 of FIG. 1, the light emitting unit 20 in the organic EL device 11 of FIG. 2, and the light emitting unit 30 in the organic EL device 12 of FIG. 3 may be referred to as an “organic layer including a light emitting layer”.

The organic EL device of one embodiment of the present invention may be a fluorescent or phosphorescent light emission-type monochromatic light emitting device or a fluorescent/phosphorescent hybrid-type white light emitting device, and may be a simple type having a single light emitting unit or a tandem type having a plurality of light emitting units. Above all, the fluorescent light emission-type device is preferred. The “light emitting unit” referred to herein refers to a minimum unit that emits light through recombination of injected holes and electrons, which includes one or more organic layers among which at least one layer is a light emitting layer.

For example, as a representative device configuration of the simple type organic EL device, the following device configuration may be exemplified.

### (1) Anode/Light Emitting Unit/Cathode

The light emitting unit may be a multilayer type having a plurality of phosphorescent light emitting layers or fluorescent light emitting layers. In this case, a space layer may intervene between the light emitting layers for the purpose of preventing excitons generated in the phosphorescent light emitting layer from diffusing into the fluorescent light emitting layer. Representative layer configurations of the simple type light emitting unit are described below. Layers in parentheses are optional.
(a) (hole injecting layer/) hole transporting layer/fluorescent light emitting layer/electron transporting layer (/electron injecting layer)
(b) (hole injecting layer/) hole transporting layer/first fluorescent light emitting layer/second fluorescent light emitting layer/electron transporting layer (/electron injecting layer)
(c) (hole injecting layer/) hole transporting layer/phosphorescent light emitting layer/space layer/fluorescent light emitting layer/electron transporting layer (/electron injecting layer)
(d) (hole injecting layer/) hole transporting layer/first phosphorescent light emitting layer/second phosphorescent light emitting layer/space layer/fluorescent light emitting layer/electron transporting layer (/electron injecting layer)
(e) (hole injecting layer/) hole transporting layer/phosphorescent light emitting layer/space layer/first fluorescent light emitting layer/second fluorescent light emitting layer/electron transporting layer (/electron injecting layer)
(f) (hole injecting layer/) hole transporting layer/electron blocking layer/fluorescent light emitting layer/electron transporting layer (/electron injecting layer)
(g) (hole injecting layer/) hole transporting layer/exciton blocking layer/fluorescent light emitting layer/electron transporting layer (/electron injecting layer)
(h) (hole injecting layer/) first hole transporting layer/second hole transporting layer/fluorescent light emitting layer/electron transporting layer (/electron injecting layer)
(i) (hole injecting layer/) first hole transporting layer/second hole transporting layer/fluorescent light emitting layer/first electron transporting layer/second electron transporting layer (/electron injecting layer)
(j) (hole injecting layer/) hole transporting layer/fluorescent light emitting layer/hole blocking layer/electron transporting layer (/electron injecting layer)
(k) (hole injecting layer/) hole transporting layer/fluorescent light emitting layer/exciton blocking layer/electron transporting layer (/electron injecting layer)

The phosphorescent and fluorescent light emitting layers may emit emission colors different from each other, respectively. Specifically, in the light emitting unit (d), a layer configuration, such as (hole injecting layer/) hole transporting layer/first phosphorescent light emitting layer (red light emission)/second phosphorescent light emitting layer (green light emission)/space layer/fluorescent light emitting layer (blue light emission)/electron transporting layer, may be exemplified.

An electron blocking layer may be properly provided between each light emitting layer and the hole transporting layer or the space layer. A hole blocking layer may be properly provided between each light emitting layer and the electron transporting layer. The employment of the electron blocking layer or the hole blocking layer allows to improve the emission efficiency by trapping electrons or holes within the light emitting layer and increasing the probability of charge recombination in the light emitting layer.

As a representative device configuration of the tandem type organic EL device, the following device configuration may be exemplified.

### (2) Anode/First Light Emitting Unit/Intermediate Layer/Second Light Emitting Unit/Cathode

For example, each of the first light emitting unit and the second light emitting unit may be independently selected from the above-described light emitting units.

The intermediate layer is also generally referred to as an intermediate electrode, an intermediate conductive layer, a charge generation layer, an electron withdrawing layer, a connecting layer, or an intermediate insulating layer, and a known material configuration can be used, in which electrons are supplied to the first light emitting unit, and holes are supplied to the second light emitting unit.

In the present invention, a host combined with a fluorescent dopant material (fluorescent light emitting material) is referred to as a fluorescent host, and a host combined with a phosphorescent dopant material is referred to as a phosphorescent host. The fluorescent host and the phosphorescent host are not distinguished only by a molecular structure. That is, the phosphorescent host means a material for forming a phosphorescent light emitting layer containing a phosphorescent dopant, and does not mean that the material cannot be used as a material for forming a fluorescent light emitting layer. The same applies to a fluorescent host.

### <Compound A>

Hereinafter, the compound A contained in the second organic EL device will be described.

The compound A contained in the hole transporting zone satisfies the above-described conditions (A) to (C).

In general, it is known that an amine compound is used as a hole transporting material, but the amine compound has low resistance to photodegradation, which is one of the causes of shortening the life of an organic EL device.

In addition, it is known that a benzoxanthene compound or a benzothioxanthene compound is used in an organic EL device. However, these compounds are mainly intended to be used as a host material of a light emitting layer, and the fact is that these compounds have not been sufficiently studied as a hole transporting material.

In particular, since a compound having a structure in which pyrene or anthracene is bonded to a benzoxanthene compound or a benzothioxanthene compound directly or via a bonding group generally has a small triplet energy T₁, there is a problem that even if the compound exhibits good performance as a host material, it is difficult to increase the efficiency as a hole transporting material.

As a result of various studies, the present inventors have found that, for example, a compound having a specific structure having a benzoxanthene skeleton or a benzothioxanthene skeleton has high excitation tolerance and high efficiency and can satisfy the above-described conditions (A) to (C), and that by using such a compound, an organic EL device in which high external quantum efficiency and a long life can be simultaneously achieved can be obtained, thereby leading to the present invention.

The highest occupied molecular orbital energy level HOMO of the compound A is preferably −5.95 to −5.60 eV, and more preferably −5.90 to −5.70 eV

The triplet energy T₁ of the compound A is preferably 2.15 eV or more, and more preferably 2.20 eV or more. On the other hand, T₁ is preferably 2.70 eV or less from the viewpoint of making it easy to avoid a case where at least one value of the HOMO or the lowest unoccupied molecular orbital energy level LUMO of the compound A is too close to a positive value. In other words, the triplet energy T₁ of the compound A is preferably 2.10 eV or more and 2.70 eV or less.

The lowest unoccupied molecular orbital energy level LUMO of the compound A is preferably −1.85 to −1.30 eV, and more preferably −1.80 to −1.45 eV

In the description herein, the LUMO is measured by cyclic voltammetry, and specifically, is measured by a method described in Examples.

The 80% attenuation time t of the PL of the compound A is preferably 0.25 h or more, more preferably 0.5 h or more, and still more preferably 1 h or more.

The compound according to one embodiment of the present invention is represented by the following formula (1′).

Hereinafter, the compound A represented by the formula (1′) and each formula included in the formula (1′) and described later may be simply referred to as “compound (1′)”.

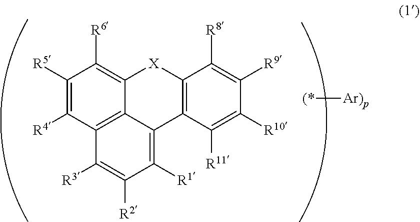

Hereinafter, symbols in the formula (1′) and respective formulae included in the formula (1′) and described later will be described. The same symbols have the same meanings. Further, in the description herein, as shown below, in the formula (1′), a partial structure bonded to * may be referred to as a “partial structure A”, and a partial structure represented by “*—Ar” may be referred to as a “partial structure B” or simply as “Ar”.

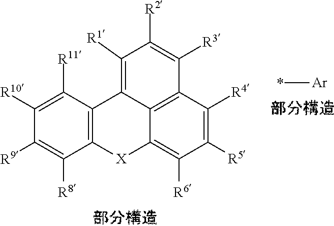

### Partial Structure A

### Partial Structure B

The partial structure A is represented by the following formula (1Aa′) or (1Ab′).

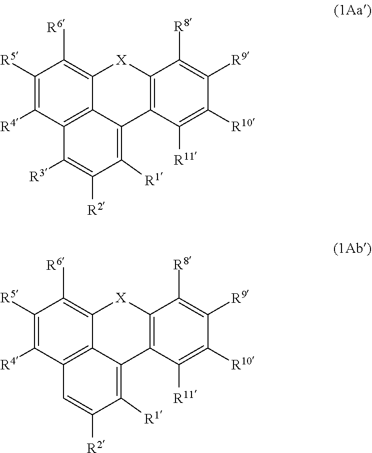

In the formulae (1′), (1Aa′), and (1Ab′), X is an oxygen atom or a sulfur atom, and preferably an oxygen atom.

In the formula (1′), p is 1, 2, or 3, preferably 1 or 2, and more preferably 1.

In the formulae (1′), (1Aa′), and (1Ab′), R^{1′} to R^{6′} and R^{8′} to R^{11′} are each independently a hydrogen atom; a substituted or unsubstituted alkyl group having 1 to 30 carbon atoms; a substituted or unsubstituted phenyl group, a substituted or unsubstituted naphthyl group, a substituted or unsubstituted biphenyl group, a substituted or unsubstituted phenanthryl group, a substituted or unsubstituted fluorenyl group, or a substituted or unsubstituted triphenylenyl group; and preferably are each independently a hydrogen atom; a substituted or unsubstituted alkyl group having 1 to 6 carbon atoms; a substituted or unsubstituted phenyl group, a substituted or unsubstituted naphthyl group, a substituted or unsubstituted biphenyl group or a substituted or unsubstituted phenanthryl group.

Provided that, when p is 1, one selected from R^{1′} to R^{6′} and R^{8′} to R^{11′} is a single bond bonded to * or a group bonded to *; when p is 2, two selected from R^{1′} to R^{6′} and R^{8′} to R^{11′} are a single bond bonded to * or a group bonded to *; and when p is 3, three selected from R^{1′} to R^{6′} and R^{8′} to R^{11′} are a single bond bonded to * or a group bonded to *.

In the formulae (1′), (1Aa′), and (1Ab′), a pair of groups adjacent to each other among R^{1′} to R^{6′} and R^{8′} to R^{11′} which are not a hydrogen atom and which are not a single bond are not bonded to each other and do not form a ring.

The unsubstituted alkyl group represented by R^{1′} to R^{6′} and R^{8′} to R^{11′} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{1′} to R^{6′} and R^{8′} to R^{11′} is preferably a phenyl group, a biphenyl group, or a naphthyl group, and more preferably a phenyl group.

In the formula (1′), Ar is
a substituted or unsubstituted phenyl group, a substituted or unsubstituted naphthyl group, a substituted or unsubstituted biphenyl group, a substituted or unsubstituted phenanthryl group, a substituted or unsubstituted fluorenyl group, or a substituted or unsubstituted triphenylenyl group, or a substituted or unsubstituted heteroaryl group (aromatic heterocyclic group) having 5 to 30 ring atoms, which is directly bonded to any of R^{1′} to R^{6′} and R^{8′} to R^{11′}; or
a substituted or unsubstituted phenyl group, a substituted or unsubstituted naphthyl group, a substituted or unsubstituted biphenyl group, a substituted or unsubstituted phenanthryl group, a substituted or unsubstituted fluorenyl group, or a substituted or unsubstituted triphenylenyl group, or a substituted or unsubstituted heteroaryl group having 5 to 30 ring atoms, bonded to a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a group formed by combining a plurality of groups selected from these divalent groups, which is bonded to any of R^{1′} to R^{6′} and R^{8′} to R^{11′}.

In one embodiment, the unsubstituted heteroaryl group having 5 to 30 ring atoms represented by Ar is a dibenzofuranyl group, a naphthobenzofuranyl group, a dinaphthofuranyl group, a dibenzothiophenyl group, a naphthobenzothiophenyl group, a dinaphthothiophenyl group, a carbazolyl group, a benzocarbazolyl group, a dibenzocarbazolyl group, a xanthenyl group, or a benzoxanthenyl group.

In the formula (1′), when p is 2 or 3, a plurality of *—Ar's are the same as or different from each other.

In one embodiment, the partial structure B (that is, *—Ar) in the formula (1′) is represented by the following formula (2A′) or (2B′).

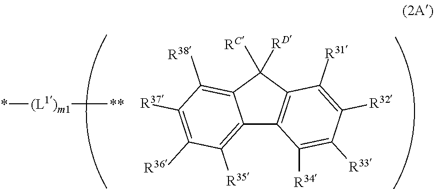

In the formula (2A′), L^{1′} is a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a divalent group formed by combining a plurality of groups selected from these groups.

In the formula (2A′), m1 is 0 or 1.

In the formula (2A′), R^{31′} to R^{38′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2A′), R^{C′} and R^{D′} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

One selected from R^{31′} to R^{38′}, R^{C′}, and R^{D′} is a single bond bonded to ** or a group bonded to **.

A pair of groups adjacent to each other among R^{31′} to R^{38′} which are not a single bond are bonded to each other to form a substituted or unsubstituted monocyclic ring, are bonded to each other to form a substituted or unsubstituted condensed ring, or are not bonded to each other and do not form a ring.

R^{C′} and R^{D′} which are not a single bond are bonded to each other to form a substituted or unsubstituted monocyclic ring, are bonded to each other to form a substituted or unsubstituted condensed ring, or are not bonded to each other and do not form a ring.

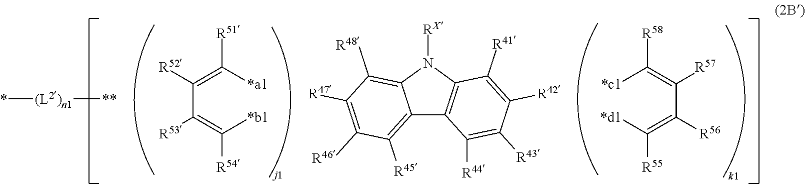

In the formula (2B′), L^{2′} is a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a divalent group formed by combining a plurality of groups selected from these groups.

In the formula (2B′), n1 is 0 or 1.

In the formula (2B′), j1 is 0 or 1.

In the formula (2B′), when j1 is 1, R^{41′} to R^{48′}, R^{51′} to R^{54′}, and R^{55′} to R^{58′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

One of R^{45′} and R^{46′}, R^{46′} and R^{47′}, or R^{47′} and R^{48′} is a single bond bonded to *a1, and the other is a single bond bonded to *b.

In the formula (2B′), k1 is 0 or 1.

In the formula (2B′), when k1 is 1, one of R^{41′} and R^{42′}, R^{42′} and R^{43′}, or R^{43′} and R^{44′} is a single bond bonded to *c1, and the other is a single bond bonded to *d1.

In the formula (2B′1. R^{X′} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

One selected from R^{41′} to R^{44′} which are not bonded to *c1 and *d1, R^{45′} to R^{48′} which are not bonded to *a1 and *b1, R^{51′} to R^{54′}, R^{55′} to R^{58′}, and R^{X′} is a single bond bonded to ** or a group bonded to **.

A pair of groups adjacent to each other among R^{41′} to R^{44′} which are not bonded to *c1 and *d1 and are not a single bond bonded to the above **, R^{45′} to R^{48′} which are not bonded to *a1 and *b1 and are not a single bond bonded to the above **, R^{51′} to R^{54′} which are not a single bond bonded to the above **, and R^{55′} to R^{58′} which are not a single bond bonded to the above ** are bonded to each other to form a substituted or unsubstituted monocyclic ring, are bonded to each other to form a substituted or unsubstituted condensed ring, or are not bonded to each other and do not form a ring.

In one embodiment, the partial structure B (that is, *—Ar) in the above formula (1) is represented by any one of the following formulae (2C′) to (2F′).

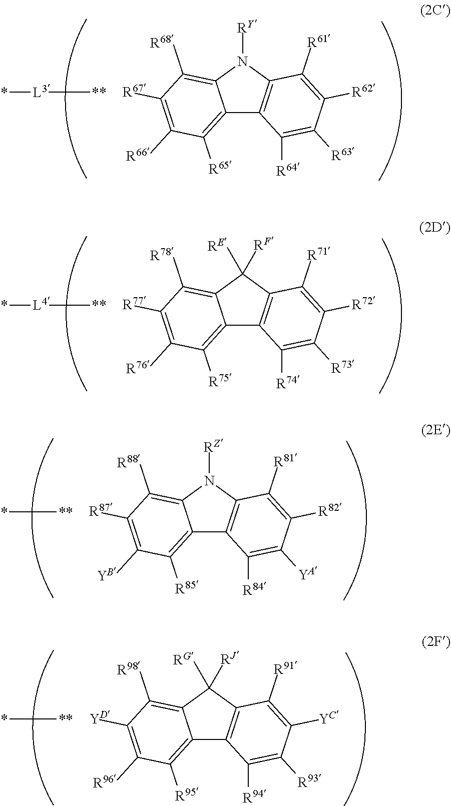

In the formula (2C′), L^{3′} is a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a divalent group formed by combining a plurality of groups selected from these groups.

In the formula (2C′), R^{61′} to R^{68′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

R^{61′} to R^{68′} are not bonded to each other and do not form a ring.

In the formula (2C′), R^{Y} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2C′), one selected from R^{61′} to R^{68′} and R^{Y} is a single bond bonded to ** or a group bonded to **.

In the formula (2D′), L^{4′} is a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a group formed by combining a plurality of groups selected from these divalent groups,

In the formula (2D′), R^{71′} to R^{78′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2D′), R^{E′} and R^{F′} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2D′), one selected from R^{71′} to R^{78′}, R^{E′}, and R^{F′} is a single bond bonded to ** or a group bonded to **.

A pair of groups adjacent to each other among R^{71′} to R^{78′} which are not a single bond are bonded to each other to form a substituted or unsubstituted monocyclic ring, are bonded to each other to form a substituted or unsubstituted condensed ring, or are not bonded to each other and do not form a ring.

R^{E′} and R^{F′} which are not a single bond are bonded to each other to form a substituted or unsubstituted monocyclic ring, are bonded to each other to form a substituted or unsubstituted condensed ring, or are not bonded to each other and do not form a ring.

In the formula (2E′), R^{81′}, R^{82′}, R^{84′}, R^{85′}, R^{87′}, R^{88′}, Y^{A′}, and Y^{B′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

R^{81′}, R^{82′}, R^{84′}, R^{85′}, R^{87′}, R^{88′}, Y^{A′}, and Y^{B′} are not bonded to each other and do not form a ring.

In the formula (2E′), R^{Z′} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2E′), one selected from R^{81′}, R^{82′}, R^{84′}, R^{85′}, R^{87′}, R^{88′}, and R^{Z′} is a single bond bonded to ** or a group bonded to **.

In the formula (2F′), R^{91′}, R^{93′} to R^{96′}, R^{98′}, Y^{C′}, and Y^{D′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms.

In the formula (2F′), R^{G′} and R^{J′} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms.

In the formula (2F′), one selected from R^{91′}, R^{93′} to R^{96′}, R^{98′}, R^{G′}, and R^{1′} is a single bond bonded to ** or a group bonded to **.

In the formula (2F′), a pair of groups adjacent to each other among R^{91′}, R^{93′} to R^{96′}, R^{98′}, and Y^{C′} and Y^{D′} which are not a single bond are bonded to each other to form a substituted or unsubstituted monocyclic ring, are bonded to each other to form a substituted or unsubstituted condensed ring, or are not bonded to each other and do not form a ring.

In the formula (2F′), R^{G} and R^{J} which are not a single bond are bonded to each other to form a substituted or unsubstituted monocyclic ring, are bonded to each other to form a substituted or unsubstituted condensed ring, or are not bonded to each other and do not form a ring.

The unsubstituted alkyl group represented by R^{31′} to R^{38′}, R^{41′} to R^{48′}, R^{51′} to R^{54′}, R^{55′} to R^{58′}, R^{61′} to R^{68′}, R^{71′} to R^{78′}, R^{88′}, R^{82′}, R^{84′}, R^{85′}, R^{87′}, R^{88′}, R^{91′}, R^{92′}, R^{94′}, R^{95′}, R^{97′}, Y^{A′}, Y^{B′} Y^{C′}, and Y^{D′} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, Y^{A′}, Y^{B′}, Y^{C′} and Y^{D′} is preferably a phenyl group, a biphenyl group, or a naphthyl group, and more preferably a phenyl group.

The unsubstituted heteroaryl group represented by R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, Y^{A′}, Y^{B′}, Y^{C′}, and Y^{D′} is preferably a pyridyl group or a quinazolinyl group.

The unsubstituted alkyl group represented by R^{C′}, R^{D′}, R^{E′}, R^{F′}, R^{G′}, and R^{J′} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{C′}, R^{D′}, R^{E′}, R^{F′}, R^{G′}, and R^{J′} is preferably a phenyl group, a biphenyl group, a naphthyl group, a terphenyl group, a phenanthrenyl group, a triphenylenyl group, or a fluorenyl group, more preferably a phenyl group, a biphenyl group, a naphthyl group, or a phenanthrenyl group, still more preferably a phenyl group, a naphthyl group, or a phenanthrenyl group, and even more preferably a phenyl group or a naphthyl group.

The unsubstituted heteroaryl group represented by R^{C′}, R^{D′}, R^{E′}, R^{F′}, R^{G′}, and R^{J′} is preferably a dibenzofuranyl group, a dibenzothiophenyl group, or a pyridyl group, and more preferably a dibenzofuranyl group or a dibenzothiophenyl group.

The unsubstituted alkyl group represented by R^{X′}, R^{Y′}, and R^{Z′} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The unsubstituted aryl group represented by R^{X′}, R^{Y′}, and R^{Z′} is preferably a phenyl group, a biphenyl group, a naphthyl group, a terphenyl group, a phenanthrenyl group, a triphenylenyl group, or a fluorenyl group, more preferably a phenyl group, a biphenyl group, a naphthyl group, or a phenanthrenyl group, still more preferably a phenyl group, a naphthyl group, or a phenanthrenyl group, and even more preferably a phenyl group or a naphthyl group.

The unsubstituted heteroaryl group represented by R^{X′}, R^{Y′}, and R^{Z′} is preferably a dibenzofuranyl group, a dibenzothiophenyl group, or a pyridyl group, and more preferably a dibenzofuranyl group or a dibenzothiophenyl group.

L^{1′} to L^{4′} are preferably each independently a substituted or unsubstituted phenylene group, a substituted or unsubstituted biphenylene group, or a substituted or unsubstituted naphthylene group.

The unsubstituted phenylene group represented by L^{1′} to L^{4′} is an o-phenylene group, a m-phenylene group, or a p-phenylene group, and particularly preferably a p-phenylene group.

The unsubstituted biphenylene group represented by L^{1′} to L^{4′} is preferably a 4,2′-biphenylene group, a 4,3′-biphenylene group, a 4,4′-biphenylene group, or a 3,3′-biphenylene group, more preferably a 4,3′-biphenylene group, a 4,4′-biphenylene group, or a 3,3′-biphenylene group, and still more preferably a 4,4′-biphenylene group.

The unsubstituted naphthylene group represented by L^{1′} to L^{4′} is preferably a 1,4-naphthylene group, a 2,6-naphthylene group, a 1,5-naphthylene group, or a 1,8-naphthylene group.

Examples of the case where L^{1′} to L^{4′} are a divalent group obtained by combining a plurality of groups selected from the above-described groups include the following groups:
a substituted or unsubstituted phenylene group and a substituted or unsubstituted naphthylene group;
a plurality of substituted or unsubstituted naphthylene groups;
a substituted or unsubstituted naphthylene group and a plurality of substituted or unsubstituted phenylene groups;
a substituted or unsubstituted phenylene group and a plurality of substituted or unsubstituted naphthylene groups; and
a plurality of substituted or unsubstituted phenylene groups and a plurality of substituted or unsubstituted naphthylene groups.

In the formula (1′),
in the case where the partial structure B is represented by the formula (2A′), it is preferable that any one of R^{31′} to R^{38′}, R^{C′}, and R^{D′} is a single bond bonded to **, a monocyclic ring or a condensed ring formed of a pair of groups adjacent to each other among R^{31′} to R^{38′} is bonded to **, or a monocyclic ring or a condensed ring formed of R^{C′} and R^{D′} is bonded to **;
in the case where the partial structure B is represented by the formula (2B′), it is preferable that one selected from R^{41′} to R^{44′} which are not bonded to *c and *d, R^{45′} to R^{48′} which are not bonded to *a1 and *b1, R^{51′} to R^{54′}, R^{55′} to R^{58′}, and R^{X′} is a single bond bonded to **, or a monocyclic ring or a condensed ring formed of a pair of groups adjacent to each other among R^{41′} to R^{44′} which are not bonded to *c1 and *d1, R^{45′} to R^{48′} which are not bonded to *a1 and *b1, R^{51′} to R^{54′}, and R^{55′} to R^{58′} is bonded to **;
in the case where the partial structure B is represented by the formula (2C′), it is preferable that one selected from R^{61′} to R^{68′}, and R^{Y} is a single bond bonded to **;
in the case where the partial structure B is represented by the formula (2D′), it is preferable that any one of R^{71′} to R^{78′}, R^{E′}, and R^{F′} which are not a hydrogen atom is a single bond bonded to **, a monocyclic ring or a condensed ring formed of a pair of groups adjacent to each other among R^{71′} to R^{78′} is bonded to **, or a monocyclic ring or a condensed ring formed of R^{E′} and R^{F′} is bonded to **;
in the case where the partial structure B is represented by the formula (2E′), it is preferable that any one of R^{81′}, R^{82′}, R^{84′}, R^{85′}, R^{87′}, R^{88′}, and R^{Z′} is a single bond bonded to **; and
in the case where *—Ar is represented by the formula (2F′), it is preferable that any one of R^{91′}, R^{93′} to R^{96′}, R^{98′}, R^{G′}, and R^{J′} which are not a hydrogen atom is a single bond bonded to the partial structure A, a monocyclic ring or a condensed ring formed of a pair of groups adjacent to each other among R^{91′}, R^{93′} to R^{96′}, R^{98′}, Y^{C′}, and Y^{D′} is bonded to the partial structure A, or a monocyclic ring or a condensed ring formed of R^{G′} and R^{J′} is bonded to the partial structure A.

In other words, in one embodiment, the compound (1′) is represented by any one of combinations of the following formulae [a′] to [1′] below.
[a′]: (1Aa′)-(2A′)
[b′]: (1Aa′)-(2B′)
[c′]: (1Aa′)-(2C′)
[d′]: (1Aa′)-(2D′)
[e′]: (1Aa′)-(2E′)
[f′]: (1Aa′)-(2F′)
[g′]: (1Ab′)-(2A′)
[h′]: (1Ab′)-(2B′)
[i′]: (1Ab′)-(2C′)
[j′]: (1Ab′)-(2D′)
[k′]: (1Ab′)-(2E′)
[1′]: (1Ab′)-(2F′)

Among these, [c′] to [h′] are preferable, and [g′] and [h′] are more preferable.

In one embodiment, the compound (1′) is represented by the following formula (1-1′) or (1-2′).

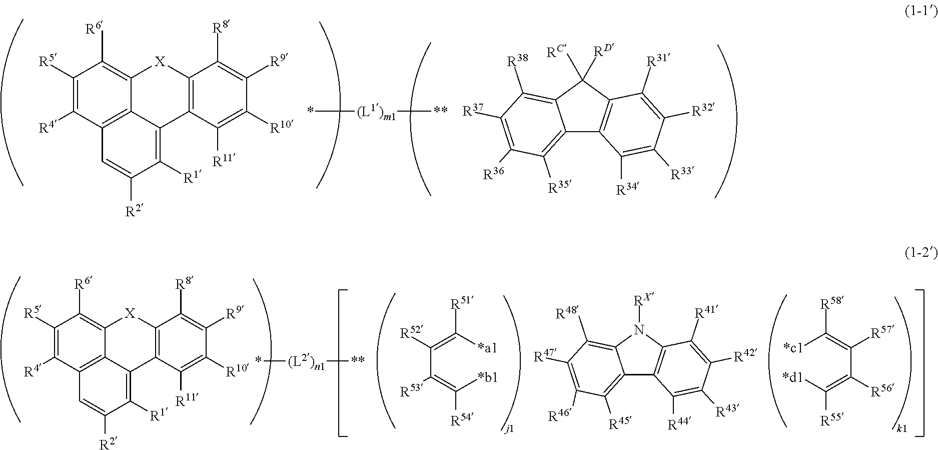

In the formulae (1-1′) and (1-2′), X, L^{1′}, L^{2′}, R^{C′}, R^{D′}, R^{X′}, R^{1′} to R^{6′}, R^{8′} to R^{11′}, R^{31′} to R^{38′}, R^{41′} to R^{48′}, R^{51′} to R^{54′}, R^{55′} to R^{58′}, j1, k1, m1, n1, *, **, *a1, *b1, *c1, and *d1 are as defined in the formulae (1′), (2A′), and (2B′).

Further, in one embodiment, the compound (1′) is represented by the following formula (1-5′) or (1-6′).

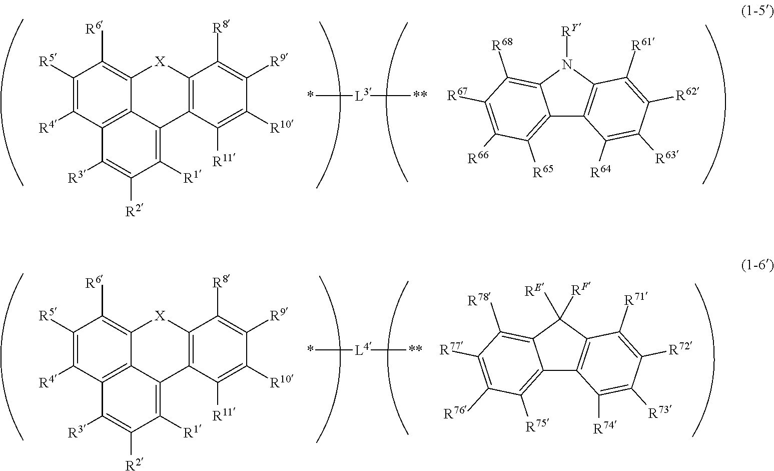

In the formulae (1-5′) and (1-6′), X, L^{3′}, L^{4′}, R^{E′}, R^{F′}, R^{Y′}, R^{1′} to R^{6′}, R^{8′} to R^{11′}, R^{61′} to R^{68′}, R^{71′} to R^{78′}, *, and ** are as defined in the formulae (1′), (2C′), and (2D′).

In one embodiment, in the formula (1-5′), one selected from R^{61′}, R^{62′}, R^{64′}, R^{65′}, R^{67′}, R^{68′}, and R^{Y′} is a single bond bonded to ** or a group bonded to **.

In one embodiment, the compound (1′) is represented by any one of the following formulae (1-5a′) to (1-5d′).

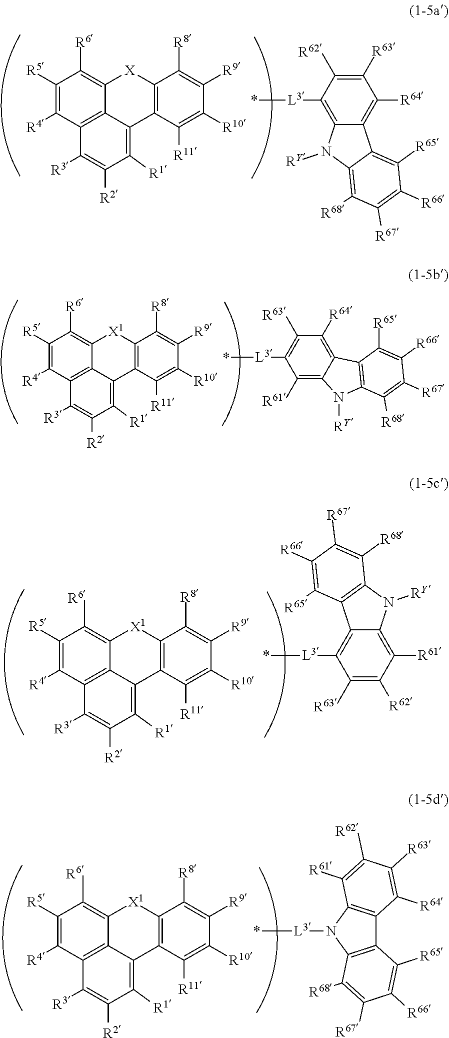

In the formulae (1-5a′) to (1-5d′), X, L^{1′}, R^{Y′}, R^{1′} to R^{6′}, R^{8′} to R^{11′}, R^{61′} to R^{68′}, and * are as defined in the formulae (1′) and (2C′).

Further, in one embodiment, in the above formula (1-6′), one selected from R^{71′}, R^{73′} to R^{76′}, R^{78′}, R^{E′}, and R^{F′} is a single bond bonded to ** or a group bonded to **.

In one embodiment, the compound (1′) is represented by any one of the following formulae (1-6a′) to (1-6c′).

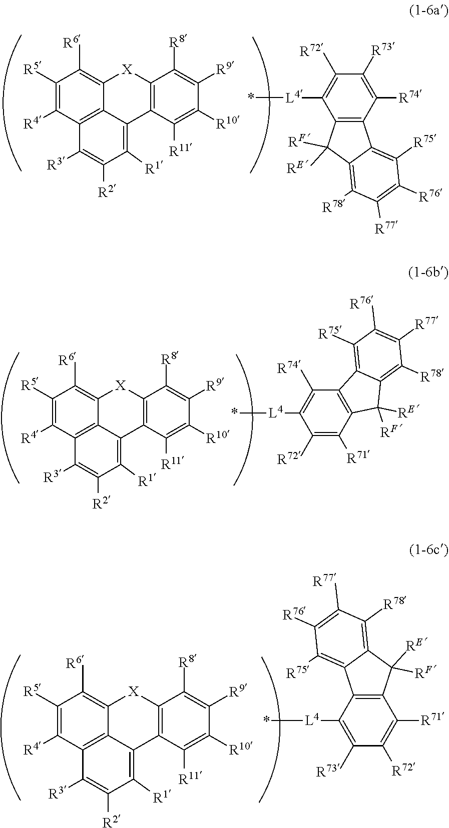

In the formulae (1-6a′) to (1-6c′), X, L^{4′}, R^{E′}, R^{F′}, R^{1′} to R^{6′}, R^{8′} to R^{11′}, R^{71′} to R^{78′}, and * are as defined in the formulae (1′) and (2D′).

Further, in one embodiment, the compound (1′) is represented by the following formula (1-9′) or (1-10′).

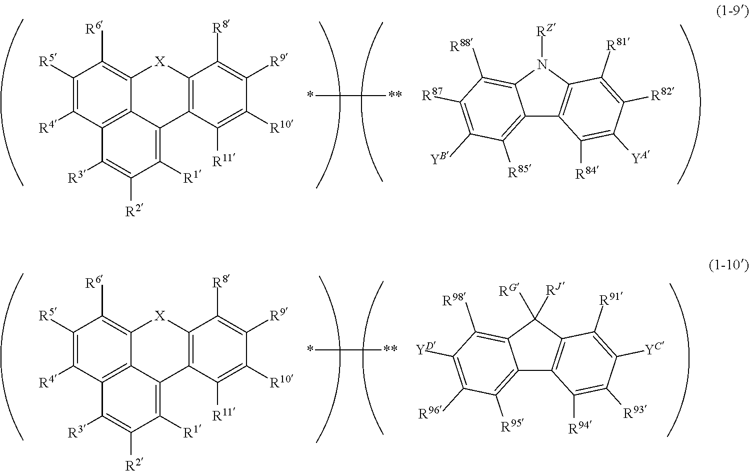

In the formulae (1-9′) and (1-10′), X, R^{G′}, R^{J′}, R^{Z′}, R^{1′} to R^{6′}, R^{8′} to R^{11′}, R^{81′}, R^{82′}, R^{84′}, R^{85′}, R^{87′}, R^{88′}, R^{91′}, R^{93′} to R^{96′}, R^{98′}, Y^{A′}, Y^{B′}, Y^{C′} Y^{D′}, *, and ** are as defined in the formulae (1′) and (2F′).

In one embodiment, the compound (1′) is represented by any one of the following formulae (1-9a′) to (1-9d′).

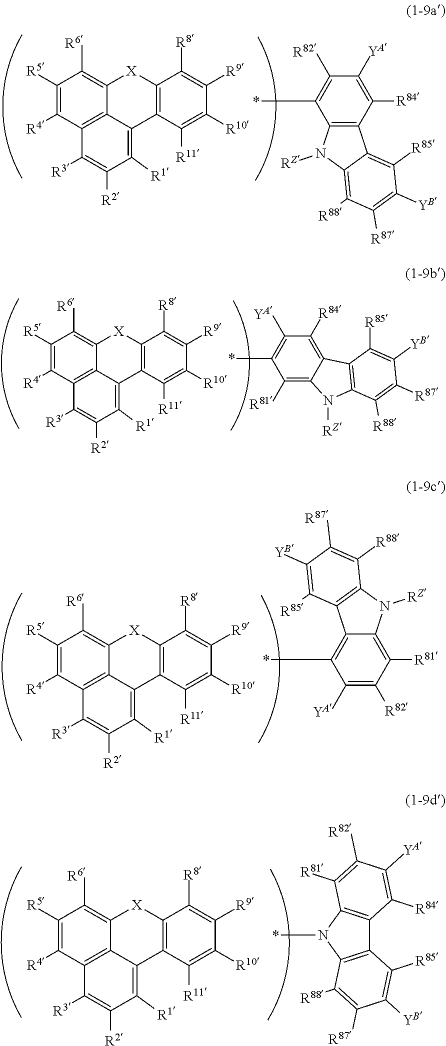

In the formulae (1-9a′) to (1-9d′), X, R^{Z′}, R^{1′} to R^{6′}, R^{8′} to R^{11′}, R^{81′}, R^{82′}, R^{84′}, R^{85′}, R^{87′}, R^{88′}, Y^{A′}, Y^{B′}, and * are as defined in the formulae (1′) and (2E′).

In one embodiment, the compound (1′) is represented by any one of the following formulae (1-10a′) to (1-10c′).

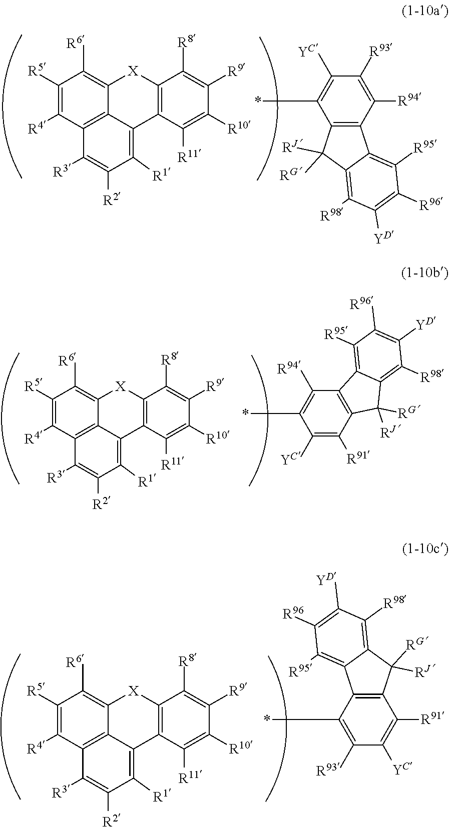

In the formulae (1-10a′) to (1-10c′), X, R^{G′}, R^{J′}, R^{1′} to R^{6′}, R^{8′} to R^{11′}, R^{91′}, R^{93′} to R^{96′}, R^{98′}, Y^{C′} Y^{D′} and * are as defined in the formulae (1′) and (2F′).

In one embodiment, the compound (1′) is represented by any of the following formulae (1-1-1′), (1-2-1′), (1-5-1′), (1-6-1′), (1-9-1′), and (1-10-1′).

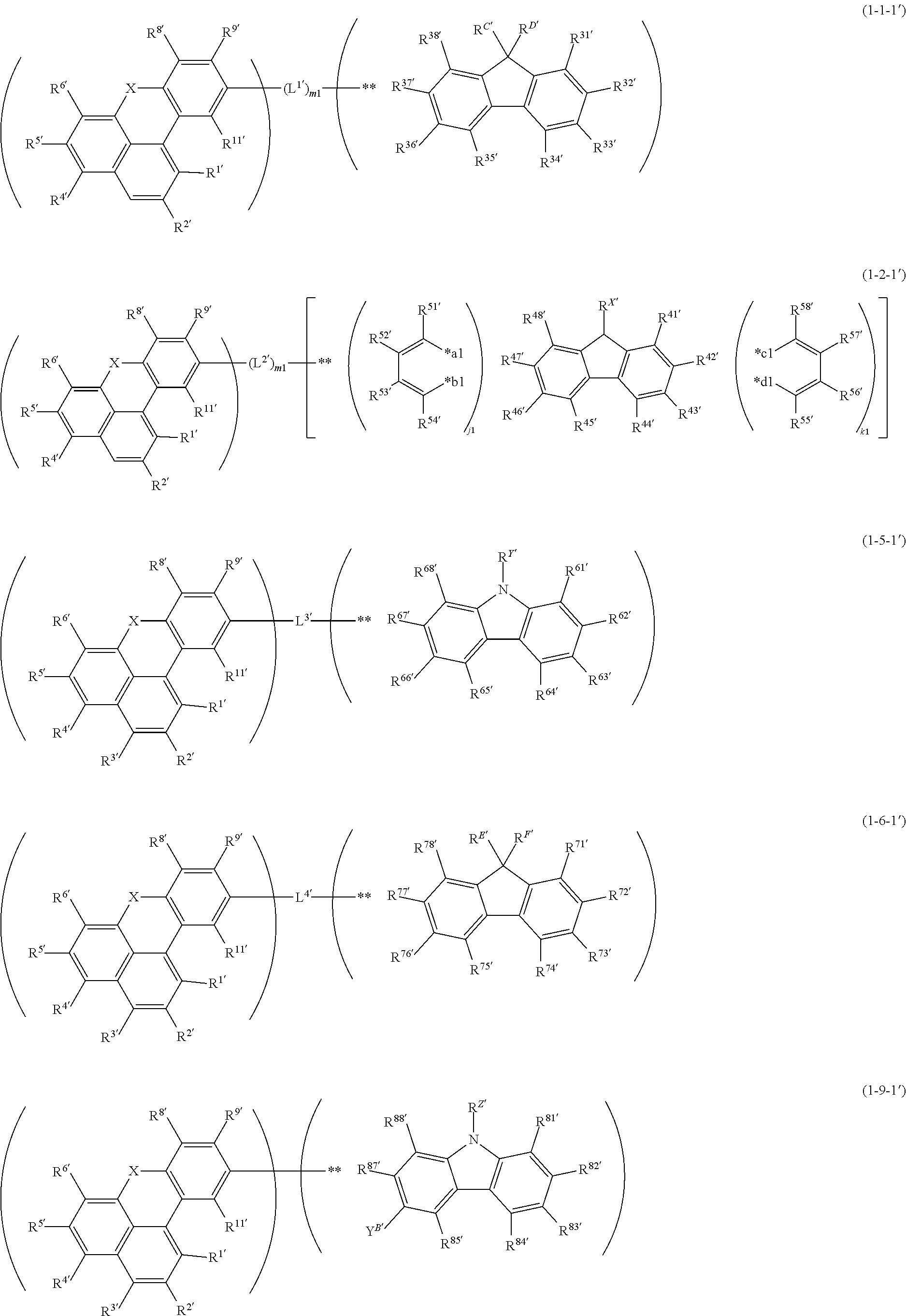

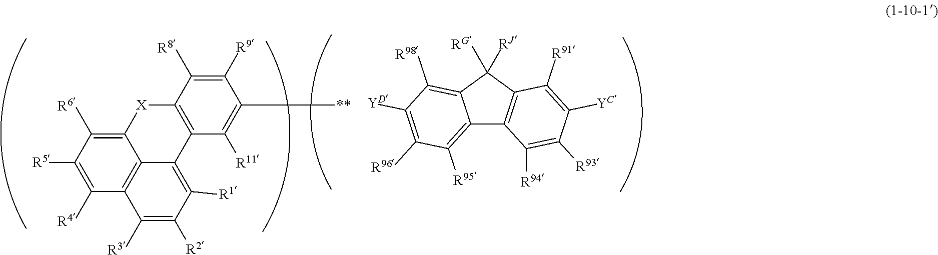

In the formulae (1-1-1′), (1-2-1′), (1-5-1′), (1-6-1′), (1-9-1′), and (1-10-1′), X, L^{1′}, L^{2′} L^{3′}, L^{4′}, R^{C′}, R^{D′}, R^{E′}, R^{F′}, R^{G′}, R^{J′}, R^{X′}, R^{Y}, R^{Z′}, R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{93′} to R^{96′}, R^{98′} Y^{A′}, Y^{B′}, Y^{C′}, Y^{D′}, j1, k1, m1, n1, **, *a1, *b1, *c1, and *d1 are as defined in the formulae (1′) and (2A′) to (2F′).

In one embodiment, in the compound A, *—Ar is represented by the formula (2A′), (1-1′), (1-3′) or (1-1-1′), and m1 is 0, or m1 is 1 and L^{1′} is a substituted or unsubstituted phenylene group or a substituted or unsubstituted biphenylene group.

In one embodiment, in the compound A, *—Ar is represented by the formula (2B′), (1-2′), (1-4′) or (1-2-1′), n1 is 0 or n1 is 1, and L^{2′} is a substituted or unsubstituted phenylene group or a substituted or unsubstituted biphenylene group.

In one embodiment, X in the compound A is an oxygen atom. To be specific, in one embodiment, the compound A is represented by the formula (1′), (1-1′), (1-2′), (1-5′), (1-6′), (1-9′), (1-10′), (1-9a′) to (1-9d′), (1-10a′) to (1-10c′), (1-1-1′), (1-2-1′), (1-5-1′), (1-6-1′), (1-9-1′), or (1-10-1′), and X is an oxygen atom.

In one embodiment, in the formula (2A′), one selected from R^{32′}, R^{34′}, R^{35′}, and R^{37′} is a single bond bonded to ** or a group bonded to **;
in the formula (2D′), one selected from R^{72′}, R^{74′}, R^{75′}, and R^{77′} is a single bond bonded to ** or a group bonded to **; and
in the formula (2F′), one selected from R^{92′}, R^{94′}, R^{95′}, and R^{97′} is a single bond bonded to ** or a group bonded to **.

In other words, in one embodiment, the partial structure B of the compound (1′) is represented by the formula (2A′), (2D′), or (2F′), and the 2-position or 4-position on the fluorene skeleton in these formulae is bonded to **.

In one embodiment, the partial structure A is represented by any one of formulae (1Aa-1′) to (1Aa-10′) obtained by replacing R¹ to R⁶ and R⁸ to R¹¹ in the formulae (1Aa-1) to (1Aa-10) described for the compound (1) with R^{1′} to R^{6′} and R^{8′} to R^{11′}.

In one embodiment, the partial structure A is represented by any one of formulae (1Aa-11′) to (1Aa-20′) obtained by replacing R¹ to R⁶ and R⁸ to R¹¹ in the formulae (1Aa-11) to (1Aa-20) described for the compound (1) with R^{1′} to R^{6′} and R^{8′} to R^{11′}.

In one embodiment, the partial structure A is represented by any one of formulae (1Ab-1′) to (1Ab-9′) obtained by replacing R¹, R² to R⁴, and R⁸ to R¹¹ in the formulae (1Ab-1) to (1Ab-9) described for the compound (1) with R^{1′}, R^{2′} to R^{4′}, and R^{8′} to R^{11′}.

In one embodiment, the partial structure A is represented by any one of formulae (1Ab-11′) to (1Ab-19′) obtained by replacing R¹, R² to R⁴, and R⁸ to R¹¹ in the formulae (1Ab-11) to (1Ab-19) described for the compound (1) with R^{1′}, R^{2′} to R^{4′}, and R^{8′} to R^{11′}.

In the formulae (1Aa-1′) to (1Aa-20′), the formulae (1Ab-1′) to (1Ab-9′), and the formulae (1Ab-11′) to (1Ab-19′), *** represents a bonding site to Ar. R^{1′} to R^{6′} and R^{8′} to R^{11′} are as defined in the formula (1′). The description of specific structural formulae of the formulae (1Aa-1′) to (1Aa-20′), the formulae (1Ab-1′) to (1Ab-9′), and the formulae (1Ab-11′) to (1Ab-19′) is omitted.

In one embodiment, in the compound A (in other words, in the formulae (2A′), (1-1′), and (1-1-1′)), R^{C′} and R^{D′} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group. Preferably, R^{C′} and R^{D′} are a substituted or unsubstituted phenyl group, one of R^{C′} and R^{D′} is a substituted or unsubstituted phenyl group, and the other is a substituted or unsubstituted naphthyl group.

In one embodiment, in the above formulae (2D′), (1-6′), and (1-6-1′), R^{E′} and R^{F′} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group. Preferably, R^{E′} and R^{F′} are a substituted or unsubstituted phenyl group, one of R^{E′} and R^{F′} is a substituted or unsubstituted phenyl group, and the other is a substituted or unsubstituted naphthyl group.

In one embodiment, in the above formulae (2F′), (1-10′), (1-10a′) to (1-10c′), and (1-10-1′), R^{G′} and R^{J′} are each independently a substituted or unsubstituted aryl group having 6 to 30 ring carbon atoms, and for example, are each independently selected from a substituted or unsubstituted phenyl group, a substituted or unsubstituted naphthyl group, and a substituted or unsubstituted phenanthryl group. Preferably, R^{G′} and R^{J′} are a substituted or unsubstituted phenyl group, one of R^{G′} and R^{J′} is a substituted or unsubstituted phenyl group, and the other is a substituted or unsubstituted naphthyl group.

In one embodiment, R^{C′} and R^{D′} in the compound A (in other words, in the formulae (2A′), (1-1′), and (1-1-1′)) are bonded to each other to form a substituted or unsubstituted monocyclic ring, or are bonded to each other to form a substituted or unsubstituted condensed ring.

In one embodiment, in the formulae (2D′), (1-6′), and (1-6-1′), R^{E′} and R^{F′} are bonded to each other to form a substituted or unsubstituted monocyclic ring, or are bonded to each other to form a substituted or unsubstituted condensed ring.

In one embodiment, in the formulae (2F′), (1-10′), (1-10a′) to (1-10c′), and (1-10-1′), R^{G′} and R^{J′} are bonded to each other to form a substituted or unsubstituted monocyclic ring, or are bonded to each other to form a substituted or unsubstituted condensed ring.

The unsubstituted monocyclic ring formed by R^{C′} and R^{D′}, the unsubstituted monocyclic ring formed by R^{E′} and R^{F′}, and the unsubstituted monocyclic ring formed by R^{G′} and R^{J′} are, for example, a benzene ring, a cyclopentane ring, or a cyclohexane ring.

The unsubstituted condensed ring formed by R^{C′} and R^{D′}, the unsubstituted condensed ring formed by R^{E′} and R^{F′}, and the unsubstituted condensed ring formed by R^{G′} and R^{J′} are, for example, a naphthalene ring or an anthracene ring.

In addition, in the case where R^{C′} and R^{D′} are bonded to each other to form an unsubstituted monocyclic ring or an unsubstituted condensed ring, in the case where R^{E′} and R^{F′} are bonded to each other to form an unsubstituted monocyclic ring or an unsubstituted condensed ring, and in the case where R^{G′} and R^{J′} are bonded to each other to form an unsubstituted monocyclic ring or an unsubstituted condensed ring, R^{C′} and R^{D′}, R^{E′} and R^{F′}, and R^{G′} and R^{J′} form a ring together with a fluorene skeleton to which these are bonded, and for example, a spirobifluorene skeleton, a spiro[9H-fluorene-9,1′-cyclopentane] skeleton, a spiro[cyclohexane-1,9′-[9H]fluorene] skeleton, and a spiro[9H-fluorene-9,2′-tricyclo[3.3.1.1³′7]decane] skeleton may be formed.

In one embodiment, the partial structure B (*—Ar) represented by the formula (2A′) is represented by any one of formulae (2Aa′) to (2Ag′) obtained by replacing R³¹ to R³⁸, L¹, and m in the formulae (2Aa) to (2Ag) described for the compound (1) with R^{31′} to R^{38′}, L^{1′}, and m1.

In one embodiment, the partial structure B (*—Ar) represented by the formula (2B′) is represented by any one of formulae (2Ba′) to (2Bg′) obtained by replacing R⁴¹ to R⁴⁸, R⁵¹ to R⁵⁴, L², and n in the formulae (2Ba) to (2Bi) described for the compound (1) with R^{41′} to R^{48′} R^{1′} to R^{54′}, L^{2′}, and n1, and the following formula (2Bj′).

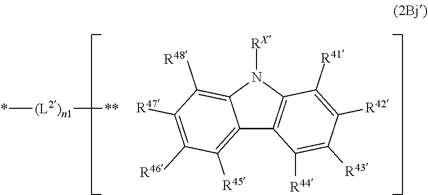

In the formula (2Bj′), L^{2′}, R^{X′}, R^{41′} to R^{48′}, n1, *, and ** are as defined in the formula (1′) and the formula (2B′).

In one embodiment, the partial structure B (*—Ar) represented by the formula (2D′) is represented by any one of formulae (2 Da′) to (2Dg′) obtained by replacing R⁷¹ to R⁷⁸, R⁴⁰¹ to R⁴⁰⁸, R⁵⁰¹ to R⁵¹⁰, and L⁴ in the formulae (2 Da) to (2Dg) described for the compound (1) with R^{71′} to R^{78′}, R^{401′} to R^{408′}, R^{501′} to R^{510′}, and L^{4′}.

In one embodiment, the partial structure B (*—Ar) represented by the formula (2F′) is represented by any one of formulae (2Fa′) to (2Fg′) obtained by replacing R⁹¹, R⁹³ to R⁹⁶, R⁹⁸, R⁶⁰¹ to R⁶⁰⁸, R⁷⁰¹ to R⁷¹⁰, Y^{C}, and Y^{D} in the formulae (2Fa) to (2Fg) described for the compound (1) with R^{91′}, R^{93′} to R^{96′}, R^{98′}, R^{601′} to R^{608′}, R^{701′} to R^{710′}, Y^{C}, and Y^{D′}

In the formulae (2Aa′) to (2Ag′), the formulae (2Ba′) to (2Bj′), the formulae (2 Da′) to (2Dg′), and the formulae (2Fa′) to (2Fg′), R^{41′} to R^{48′}, R^{51′} to R^{54′}, R^{58′} to R^{58′}, R^{71′} to R^{78′}, R^{91′} to R^{98′}, R^{X}, L², L⁴, and n are as defined in the formulae (2A′), (2B′), and (2D′) to (2F′). Preferred examples of each group are also as described in the formulae (2A′), (2B′), and (2D′) to (2F′). Note that description of specific structural formulae of the formulae (2Aa′) to (2Ag′), the formulae (2Ba′) to (2Bj′), the formulae (2 Da′) to (2Dg′), and the formulae (2Fa′) to (2Fg′) is omitted.

It is preferable that the compound A does not include a pyrene skeleton, an anthracene skeleton, a fluoranthene skeleton, a benzotriphenylene skeleton, a benzoanthracene skeleton, a benzopyrene skeleton, a benzofluoranthene skeleton, a chrysene skeleton, and a benzophenanthrene skeleton.

In one embodiment of the compound A, at least one of the following (1′) to (19′) is a deuterium atom:
(1′) a hydrogen atom represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R⁸¹′, R^{82′}, R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R^{71′} to R^{78′}, R^{401′} to R^{408′}, R^{501′} to R^{510′}, R^{601′} to R⁶⁰⁸, R^{701′} to R^{710′}, Y^{A′}, Y^{B′}, Y^{C′}, and Y^{D′};
(2′) a hydrogen atom at the 3-position of the benzoxanthene skeleton of the formula (1Ab′);
(3′) a hydrogen atom directly bonded to an alkyl group represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′} R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R^{71′} to R^{78′}, R^{401′} to R^{408′}, R^{501′} to R^{510′}, R^{601′} to R^{608′}, R^{701′} to R^{710′}, Y^{A′}, Y^{B′}, Y^{C′}, and Y^{D′};
(4′) a hydrogen atom directly bonded to an aryl group represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R^{71′} to R^{78′}, R^{401′} to R^{408′}, R^{501′} to R^{510′}, R^{601′} to R⁶⁰⁸, R^{701′} to R^{710′}, Y^{A′}, Y^{B′}, Y^{C′} and Y^{D′};
(5′) a hydrogen atom directly bonded to a heteroaryl group represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R^{71′} to R^{78′}, R^{401′} to R^{408′}, R^{501′} to R^{510′}, R^{601′} to R^{608′}, R^{701′} to R^{710′}, Y^{A′}, Y^{B′}, Y^{C′}, and Y^{D′};
(6′) a hydrogen atom directly bonded to a substituent of an alkyl group represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R^{71′} to R^{78′}, R^{401′} to R^{408′}, R^{501′} to R^{510′}, R^{601′} to R^{608′}, R^{701′} to R^{710′}, Y^{A′}, Y^{B′}, Y^{C′}, and Y^{D′};
(7′) a hydrogen atom directly bonded to a substituent of an aryl group represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R⁷¹ to R^{78′}, R⁴⁰¹ to R^{408′}, R⁵⁰¹ to R^{51′}, R^{601′} to R^{608′}, R^{701′} to R^{710′}, Y^{A′}, Y^{B′}, Y^{C′}, and Y^{D′};
(8′) a hydrogen atom directly bonded to a substituent of a heteroaryl group represented by each of R^{1′} to R^{6′}, R^{8′} to R^{11′}, R^{31′} to R^{38′}, R^{41′} to R^{48′}, R^{51′} to R^{54′}, R^{55′} to R^{58′}, R^{61′} to R^{68′}, R^{71′} to R^{78′}, R^{81′}, R^{82′}, R^{84′}, R^{85′}, R^{87′}, R^{88′}, R^{91′}, R^{92′}, R^{94′}, R^{95′}, R^{97′}, R^{98′}, R^{201′} to R^{208′}, R^{301′} to R^{310′}, R^{71′} to R^{78′}, R^{401′} to R^{408′}, R^{501′} to R^{510′}, R^{601′} to R^{608′}, R^{701′} to R^{710′}, Y^{A′}, Y^{B′}, Y^{C′}, and Y^{D′};
(9′) a hydrogen atom represented by each of R^{C′}, R^{D′}, R^{E′}, R^{F′}, R^{G′}, R^{J′}, R^{X′}, R^{Y′}, and R^{Z′};
(10′) a hydrogen atom directly bonded to an alkyl group represented by each of R^{C′}, R^{D′}, R^{E′}, R^{F′}, R^{G′}, R^{J′}, R^{X′}, R^{Y′}, and R^{Z′};
(11′) a hydrogen atom directly bonded to an aryl group represented by each of R^{C′}, R^{D}, R^{E′}, R^{F′}, R^{G′}, R^{J′}, R^{X′}, R^{Y′} and R^{Z′};
(12′) a hydrogen atom directly bonded to a heteroaryl group represented by each of R^{C′}, R^{D′}, R^{E′}, R^{F′}, R^{G′}, R^{Y}, R^{X′}, R^{Y′}, and R^{Z′};
(13′) a hydrogen atom directly bonded to a substituent of an alkyl group represented by each of R^{Y}, R^{D′}, R^{E′}, R^{F′}, R^{G′}, R^{Y}, R^{X′}, R^{Y′}, and R^{Z′};
(14′) a hydrogen atom directly bonded to a substituent of an aryl group represented by each of R^{C′}, R^{D′}, R^{E′}, R^{F′}, R^{G′}, R^{Y}, R^{X′}, R^{Y′}, and R^{Z′};
(15′) a hydrogen atom directly bonded to a substituent of a heteroaryl group represented by each of R^{C′}, R^{D′}, R^{E′}, R^{F′}, R^{G′}, R^{J′}, R^{X′}, R^{Y′}, and R^{Z′};
(16′) a hydrogen atom directly bonded to an arylene group represented by each of L^{1′}, L^{2′}, L^{3′}, and L^{4′};
(17′) a hydrogen atom directly bonded to a divalent heterocyclic group represented by each of L^{1′}, L^{2′}, L^{3′}, and L^{4′};
(18′) a hydrogen atom directly bonded to a substituent of an arylene group represented by each of L^{1′}, L^{2′}, L^{3′}, and L^{4′}; and
(19′) a hydrogen atom directly bonded to a substituent of a divalent heterocyclic group represented by each of L^{1′}, L^{2′}, L^{3′}, and L^{4′}.

In one embodiment, the compound A contains at least one deuterium atom.

The compound A may contain a naturally-derived deuterium atom, or a deuterium atom may be intentionally introduced into the compound A by using a deuterated compound as a part or the whole of the raw material compound.

The preferred deuteration rate of the compound A is the same as that described for the inventive compound.

In addition, the compound A may be a mixture containing a deuterated compound and a non-deuterated compound, or a mixture of two or more compounds having different deuteration rates from each other, and the preferable numerical range of the deuteration rate of the mixture in this case is the same as that described for the above-mentioned inventive compound.

Further, the respective proportions of the number of the deuterium atoms to the number of all the hydrogen atoms in the compound A are also the same as those described for the above-mentioned inventive compound.

The deuteration rate of the compound A depends on the deuteration rate of the raw material compound used. Even when a raw material having a predetermined deuteration rate is used, a naturally-derived protium isotope can be contained in a certain ratio. Accordingly, an embodiment of the deuteration rate of the compound A shown below includes the proportion for which a minor amount of a naturally-derived isotope is taken into consideration, relative to the proportion determined by counting the number of the deuterium atoms merely represented by a chemical formula.

The deuteration rate of the compound A is preferably 1% or more, more preferably 3% or more, still more preferably 5% or more, even more preferably 10% or more, and even more preferably 50% or more.

The compound A may be a deuterium compound in which all hydrogen atoms are deuterium atoms (that is, the deuteration rate of the compound A is 100%).

The compound A may be a mixture of a deuterated compound and a non-deuterated compound, or a mixture of two or more compounds having different deuteration rates from each other. The deuteration rate of the mixture is preferably 1% or more, more preferably 3% or more, still more preferably 5% or more, even more preferably 10% or more, and even more preferably 50% or more, and is less than 100%.

The proportion of the number of the deuterium atoms to the number of all the hydrogen atoms in the compound A is preferably 1% or more, more preferably 3% or more, still more preferably 5% or more, even more preferably 10% or more, and is 100% or less.

Specific examples of the compound A will be described below, but the compound A is not limited to the following example compounds.

In the following specific examples, D represents a deuterium atom.

Compounds obtained by excluding compounds having a structure represented by the above formula (1B) from the exemplified compounds of the above compound (1).

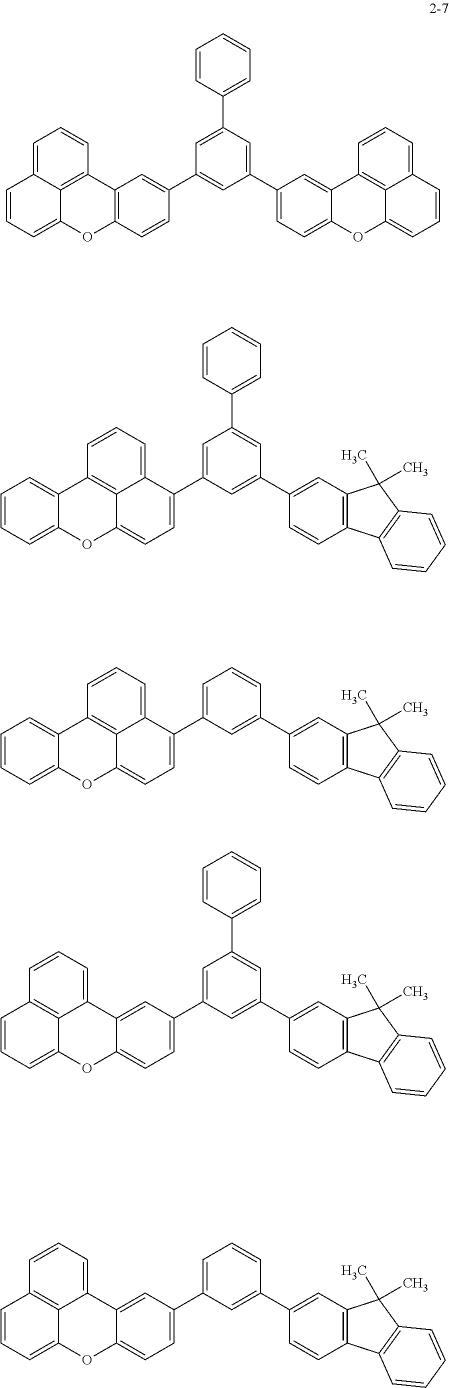 [SMILES: c1ccc(-c2cc(-c3ccc4c(c3)-c3cccc5cccc(c35)O4)cc(-c3ccc4c(c3)-c3cccc5cccc(c35)O4)c2)cc1 ; CC1(C)c2ccccc2-c2ccc(-c3cc(-c4ccccc4)cc(-c4ccc5c6c(cccc46)-c4ccccc4O5)c3)cc21 ; CC1(C)c2ccccc2-c2ccc(-c3cccc(-c4ccc5c6c(cccc46)-c4ccccc4O5)c3)cc21 ; CC1(C)c2ccccc2-c2ccc(-c3cc(-c4ccccc4)cc(-c4ccc5c(c4)-c4cccc6cccc(c46)O5)c3)cc21 ; CC1(C)c2ccccc2-c2ccc(-c3cccc(-c4ccc5c(c4)-c4cccc6cccc(c46)O5)c3)cc21]

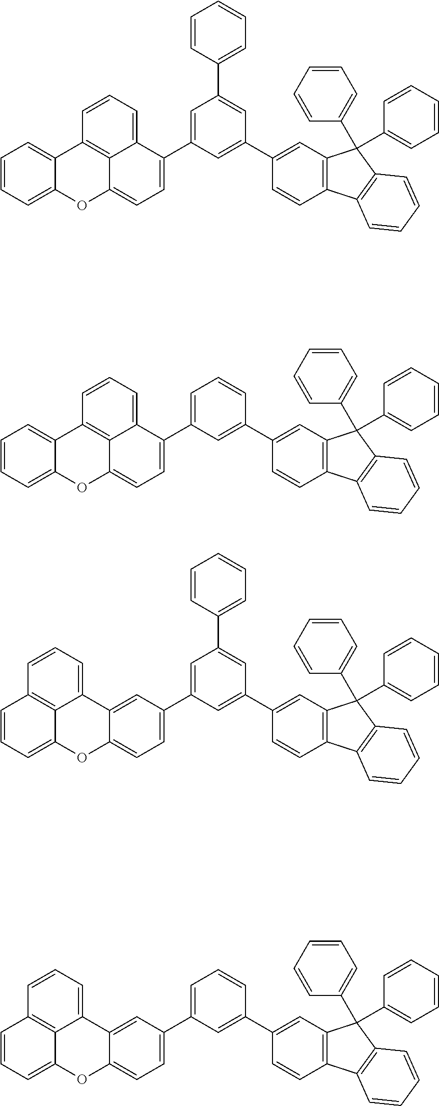 [SMILES: c1ccc(-c2cc(-c3ccc4c(c3)C(c3ccccc3)(c3ccccc3)c3ccccc3-4)cc(-c3ccc4c5c(cccc35)-c3ccccc3O4)c2)cc1 ; c1ccc(C2(c3ccccc3)c3ccccc3-c3ccc(-c4cccc(-c5ccc6c7c(cccc57)-c5ccccc5O6)c4)cc32)cc1 ; c1ccc(-c2cc(-c3ccc4c(c3)-c3cccc5cccc(c35)O4)cc(-c3ccc4c(c3)C(c3ccccc3)(c3ccccc3)c3ccccc3-4)c2)cc1 ; c1ccc(C2(c3ccccc3)c3ccccc3-c3ccc(-c4cccc(-c5ccc6c(c5)-c5cccc7cccc(c57)O6)c4)cc32)cc1]

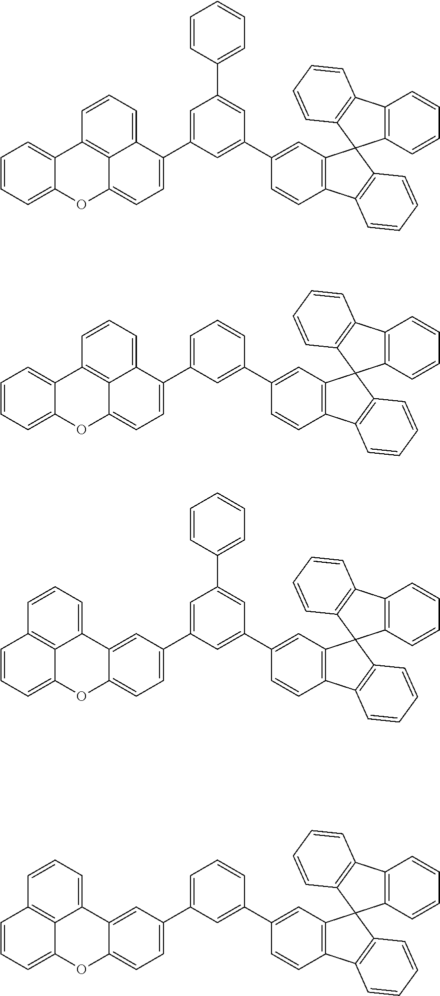 [SMILES: c1ccc(-c2cc(-c3ccc4c(c3)C3(c5ccccc5-c5ccccc53)c3ccccc3-4)cc(-c3ccc4c5c(cccc35)-c3ccccc3O4)c2)cc1 ; c1cc(-c2ccc3c(c2)C2(c4ccccc4-c4ccccc42)c2ccccc2-3)cc(-c2ccc3c4c(cccc24)-c2ccccc2O3)c1 ; c1ccc(-c2cc(-c3ccc4c(c3)-c3cccc5cccc(c35)O4)cc(-c3ccc4c(c3)C3(c5ccccc5-c5ccccc53)c3ccccc3-4)c2)cc1 ; c1cc(-c2ccc3c(c2)-c2cccc4cccc(c24)O3)cc(-c2ccc3c(c2)C2(c4ccccc4-c4ccccc42)c2ccccc2-3)c1]

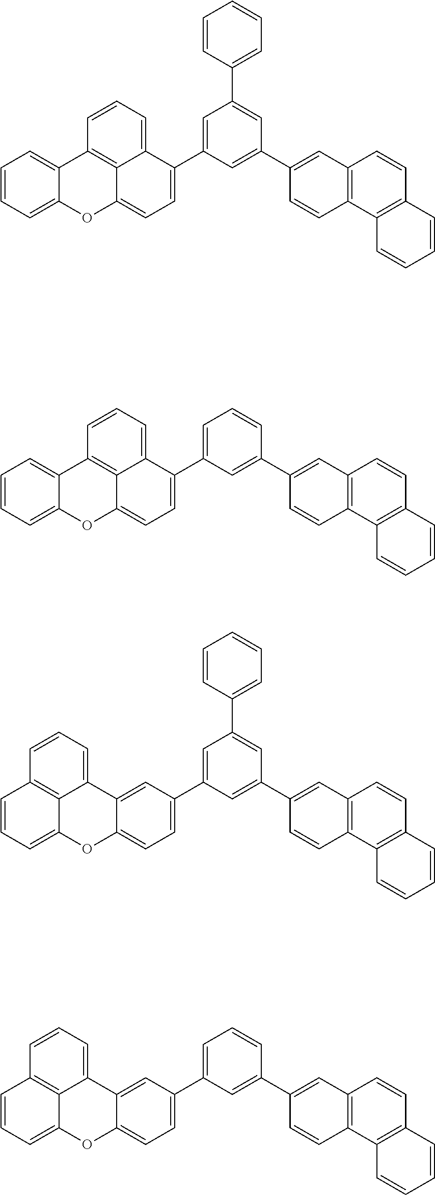 [SMILES: c1ccc(-c2cc(-c3ccc4c(ccc5ccccc54)c3)cc(-c3ccc4c5c(cccc35)-c3ccccc3O4)c2)cc1 ; c1cc(-c2ccc3c(ccc4ccccc43)c2)cc(-c2ccc3c4c(cccc24)-c2ccccc2O3)c1 ; c1ccc(-c2cc(-c3ccc4c(c3)-c3cccc5cccc(c35)O4)cc(-c3ccc4c(ccc5ccccc54)c3)c2)cc1 ; c1cc(-c2ccc3c(c2)-c2cccc4cccc(c24)O3)cc(-c2ccc3c(ccc4ccccc43)c2)c1]

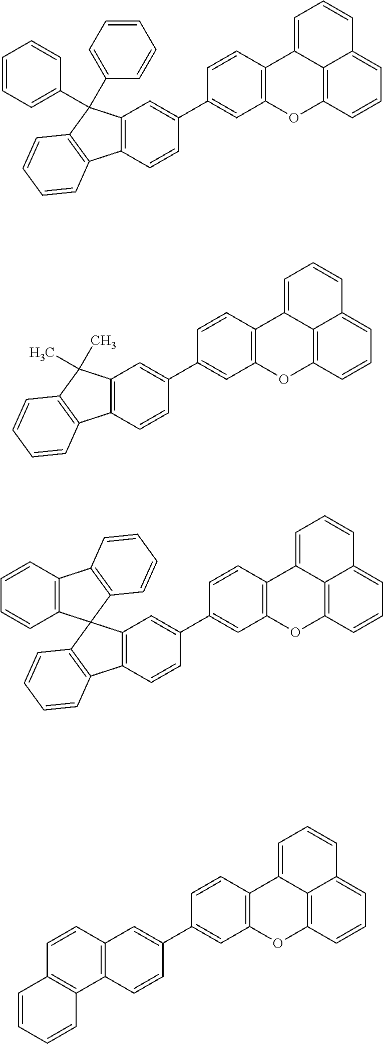 [SMILES: c1ccc(C2(c3ccccc3)c3ccccc3-c3ccc(-c4ccc5c(c4)Oc4cccc6cccc-5c46)cc32)cc1 ; CC1(C)c2ccccc2-c2ccc(-c3ccc4c(c3)Oc3cccc5cccc-4c35)cc21 ; c1ccc2c(c1)-c1ccccc1C21c2ccccc2-c2ccc(-c3ccc4c(c3)Oc3cccc5cccc-4c35)cc21 ; c1cc2c3c(cccc3c1)-c1ccc(-c3ccc4c(ccc5ccccc54)c3)cc1O2]

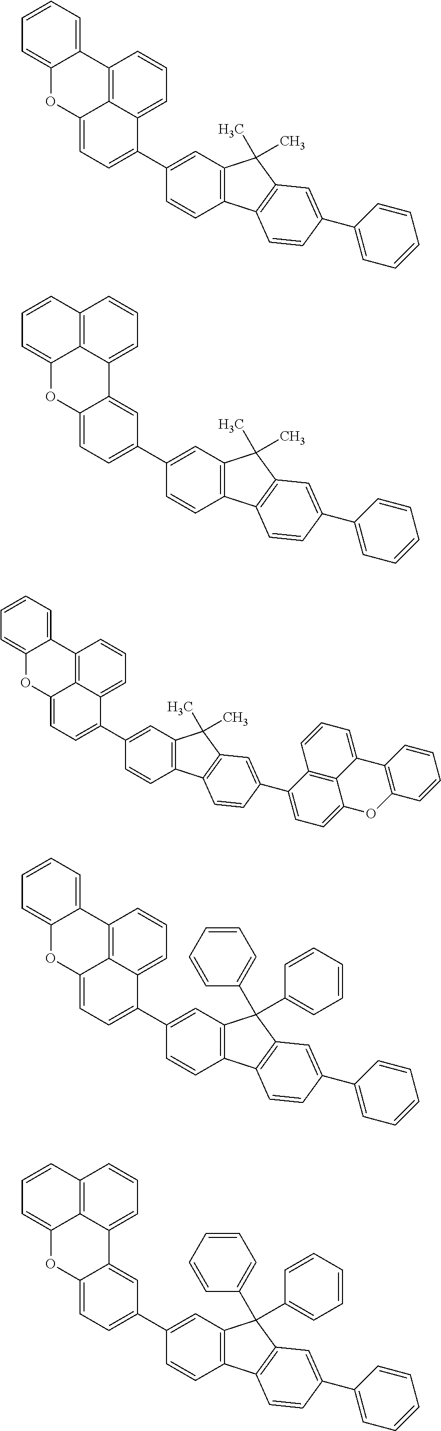 [SMILES: CC1(C)c2cc(-c3ccccc3)ccc2-c2ccc(-c3ccc4c5c(cccc35)-c3ccccc3O4)cc21 ; CC1(C)c2cc(-c3ccccc3)ccc2-c2ccc(-c3ccc4c(c3)-c3cccc5cccc(c35)O4)cc21 ; CC1(C)c2cc(-c3ccc4c5c(cccc35)-c3ccccc3O4)ccc2-c2ccc(-c3ccc4c5c(cccc35)-c3ccccc3O4)cc21 ; c1ccc(-c2ccc3c(c2)C(c2ccccc2)(c2ccccc2)c2cc(-c4ccc5c6c(cccc46)-c4ccccc4O5)ccc2-3)cc1 ; c1ccc(-c2ccc3c(c2)C(c2ccccc2)(c2ccccc2)c2cc(-c4ccc5c(c4)-c4cccc6cccc(c46)O5)ccc2-3)cc1]

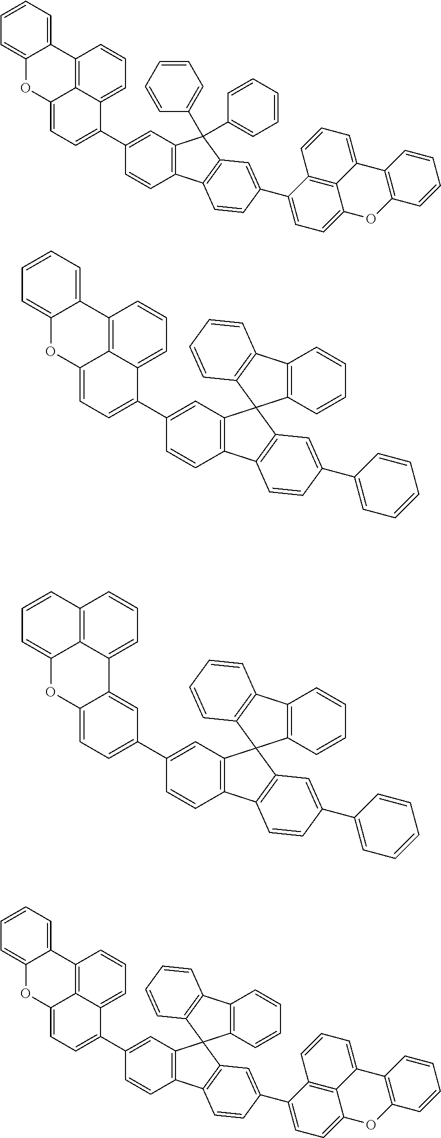 [SMILES: c1ccc(C2(c3ccccc3)c3cc(-c4ccc5c6c(cccc46)-c4ccccc4O5)ccc3-c3ccc(-c4ccc5c6c(cccc46)-c4ccccc4O5)cc32)cc1 ; c1ccc(-c2ccc3c(c2)C2(c4ccccc4-c4ccccc42)c2cc(-c4ccc5c6c(cccc46)-c4ccccc4O5)ccc2-3)cc1 ; c1ccc(-c2ccc3c(c2)C2(c4ccccc4-c4ccccc42)c2cc(-c4ccc5c(c4)-c4cccc6cccc(c46)O5)ccc2-3)cc1 ; c1ccc2c(c1)Oc1ccc(-c3ccc4c(c3)C3(c5ccccc5-c5ccccc53)c3cc(-c5ccc6c7c(cccc57)-c5ccccc5O6)ccc3-4)c3cccc-2c13]

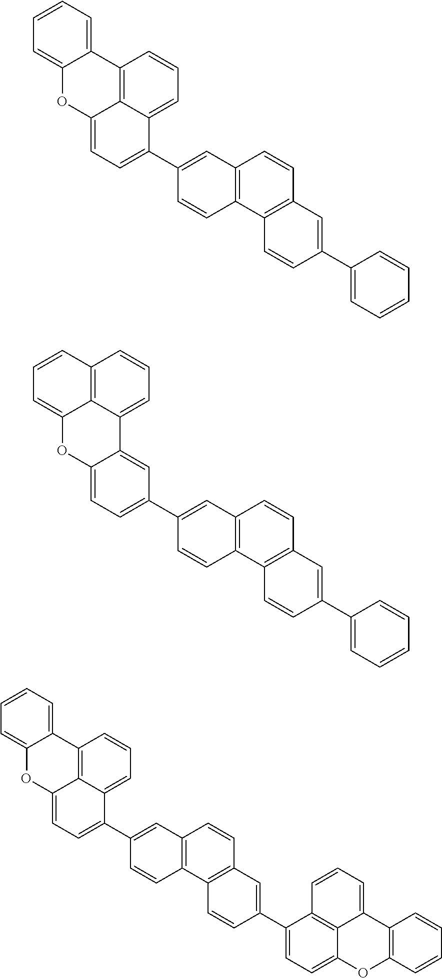 [SMILES: c1ccc(-c2ccc3c(ccc4cc(-c5ccc6c7c(cccc57)-c5ccccc5O6)ccc43)c2)cc1 ; c1ccc(-c2ccc3c(ccc4cc(-c5ccc6c(c5)-c5cccc7cccc(c57)O6)ccc43)c2)cc1 ; c1ccc2c(c1)Oc1ccc(-c3ccc4c(ccc5cc(-c6ccc7c8c(cccc68)-c6ccccc6O7)ccc54)c3)c3cccc-2c13]

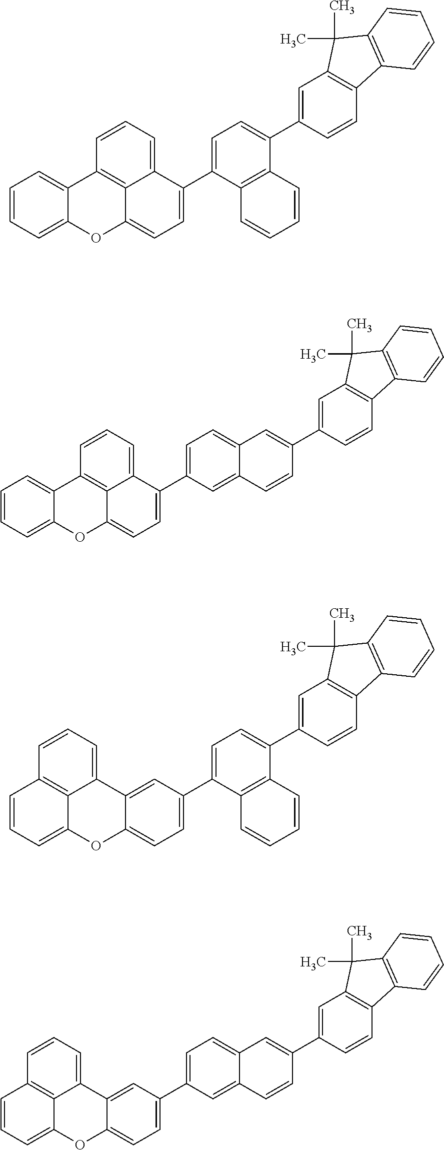 [SMILES: CC1(C)c2ccccc2-c2ccc(-c3ccc(-c4ccc5c6c(cccc46)-c4ccccc4O5)c4ccccc34)cc21 ; CC1(C)c2ccccc2-c2ccc(-c3ccc4cc(-c5ccc6c7c(cccc57)-c5ccccc5O6)ccc4c3)cc21 ; CC1(C)c2ccccc2-c2ccc(-c3ccc(-c4ccc5c(c4)-c4cccc6cccc(c46)O5)c4ccccc34)cc21 ; CC1(C)c2ccccc2-c2ccc(-c3ccc4cc(-c5ccc6c(c5)-c5cccc7cccc(c57)O6)ccc4c3)cc21]

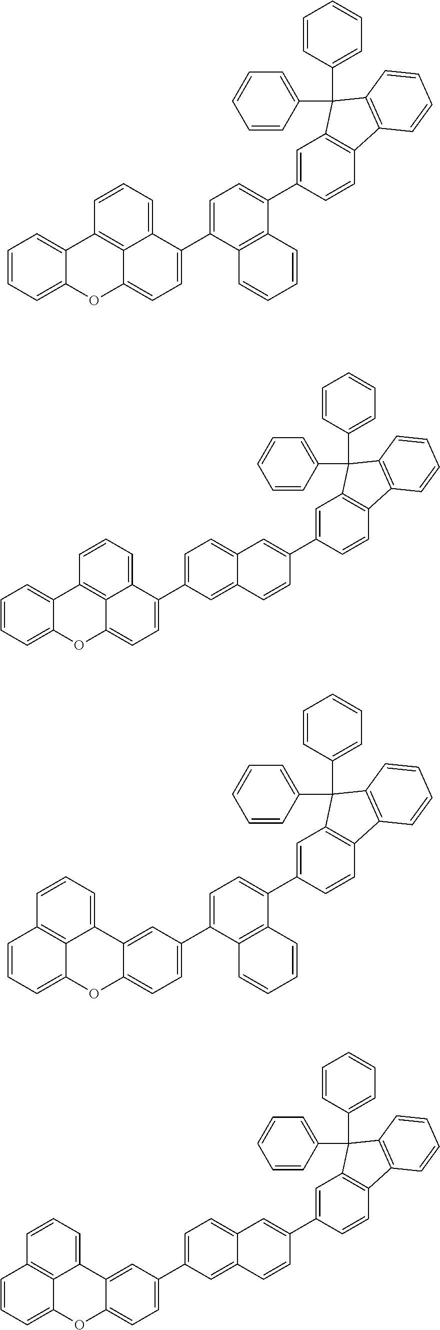 [SMILES: c1ccc(C2(c3ccccc3)c3ccccc3-c3ccc(-c4ccc(-c5ccc6c7c(cccc57)-c5ccccc5O6)c5ccccc45)cc32)cc1 ; c1ccc(C2(c3ccccc3)c3ccccc3-c3ccc(-c4ccc5cc(-c6ccc7c8c(cccc68)-c6ccccc6O7)ccc5c4)cc32)cc1 ; c1ccc(C2(c3ccccc3)c3ccccc3-c3ccc(-c4ccc(-c5ccc6c(c5)-c5cccc7cccc(c57)O6)c5ccccc45)cc32)cc1 ; c1ccc(C2(c3ccccc3)c3ccccc3-c3ccc(-c4ccc5cc(-c6ccc7c(c6)-c6cccc8cccc(c68)O7)ccc5c4)cc32)cc1]

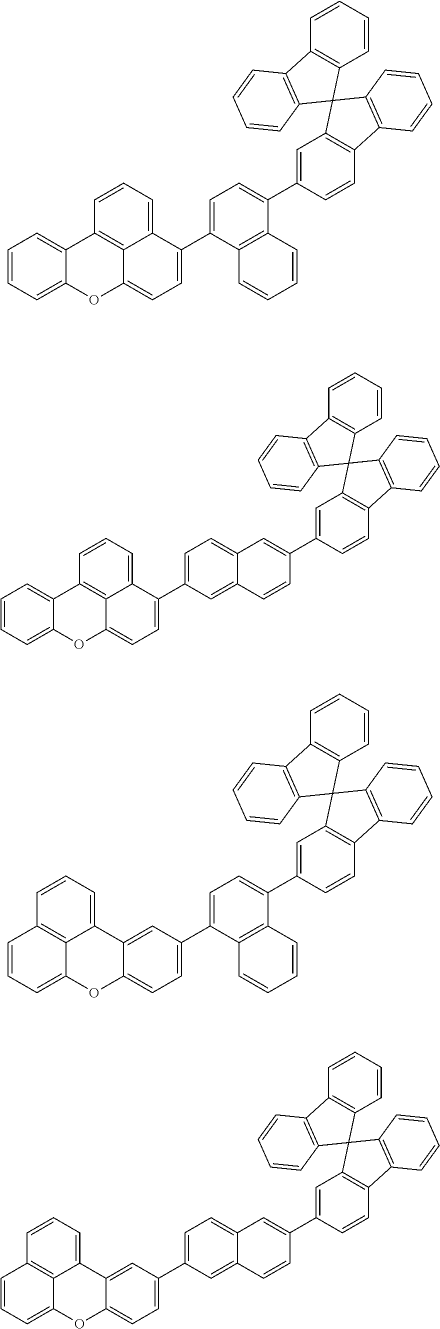 [SMILES: c1ccc2c(c1)Oc1ccc(-c3ccc(-c4ccc5c(c4)C4(c6ccccc6-c6ccccc64)c4ccccc4-5)c4ccccc34)c3cccc-2c13 ; c1ccc2c(c1)Oc1ccc(-c3ccc4cc(-c5ccc6c(c5)C5(c7ccccc7-c7ccccc75)c5ccccc5-6)ccc4c3)c3cccc-2c13 ; c1ccc2c(c1)-c1ccccc1C21c2ccccc2-c2ccc(-c3ccc(-c4ccc5c(c4)-c4cccc6cccc(c46)O5)c4ccccc34)cc21 ; c1ccc2c(c1)-c1ccccc1C21c2ccccc2-c2ccc(-c3ccc4cc(-c5ccc6c(c5)-c5cccc7cccc(c57)O6)ccc4c3)cc21]

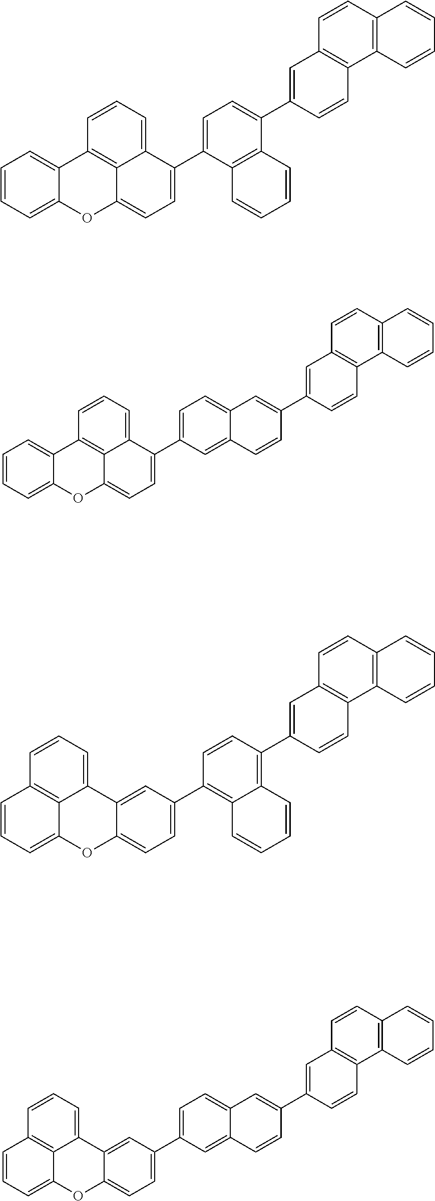 [SMILES: c1ccc2c(c1)Oc1ccc(-c3ccc(-c4ccc5c(ccc6ccccc65)c4)c4ccccc34)c3cccc-2c13 ; c1ccc2c(c1)Oc1ccc(-c3ccc4cc(-c5ccc6c(ccc7ccccc76)c5)ccc4c3)c3cccc-2c13 ; c1cc2c3c(cccc3c1)-c1cc(-c3ccc(-c4ccc5c(ccc6ccccc65)c4)c4ccccc34)ccc1O2 ; c1cc2c3c(cccc3c1)-c1cc(-c3ccc4cc(-c5ccc6c(ccc7ccccc76)c5)ccc4c3)ccc1O2]

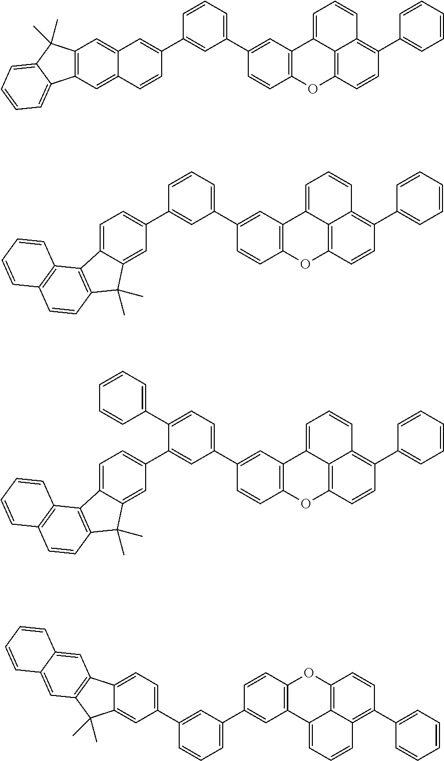 [SMILES: CC1(C)c2ccccc2-c2cc3ccc(-c4cccc(-c5ccc6c(c5)-c5cccc7c(-c8ccccc8)ccc(c57)O6)c4)cc3cc21 ; CC1(C)c2cc(-c3cccc(-c4ccc5c(c4)-c4cccc6c(-c7ccccc7)ccc(c46)O5)c3)ccc2-c2c1ccc1ccccc21 ; CC1(C)c2cc(-c3cc(-c4ccc5c(c4)-c4cccc6c(-c7ccccc7)ccc(c46)O5)ccc3-c3ccccc3)ccc2-c2c1ccc1ccccc21 ; CC1(C)c2cc(-c3cccc(-c4ccc5c(c4)-c4cccc6c(-c7ccccc7)ccc(c46)O5)c3)ccc2-c2cc3ccccc3cc21]

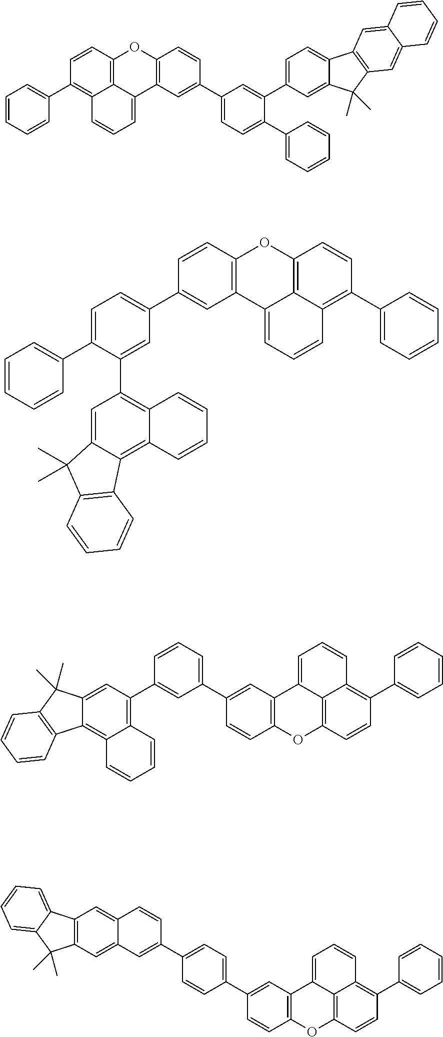 [SMILES: CC1(C)c2cc(-c3cc(-c4ccc5c(c4)-c4cccc6c(-c7ccccc7)ccc(c46)O5)ccc3-c3ccccc3)ccc2-c2cc3ccccc3cc21 ; CC1(C)c2ccccc2-c2c1cc(-c1cc(-c3ccc4c(c3)-c3cccc5c(-c6ccccc6)ccc(c35)O4)ccc1-c1ccccc1)c1ccccc21 ; CC1(C)c2ccccc2-c2c1cc(-c1cccc(-c3ccc4c(c3)-c3cccc5c(-c6ccccc6)ccc(c35)O4)c1)c1ccccc21 ; CC1(C)c2ccccc2-c2cc3ccc(-c4ccc(-c5ccc6c(c5)-c5cccc7c(-c8ccccc8)ccc(c57)O6)cc4)cc3cc21]

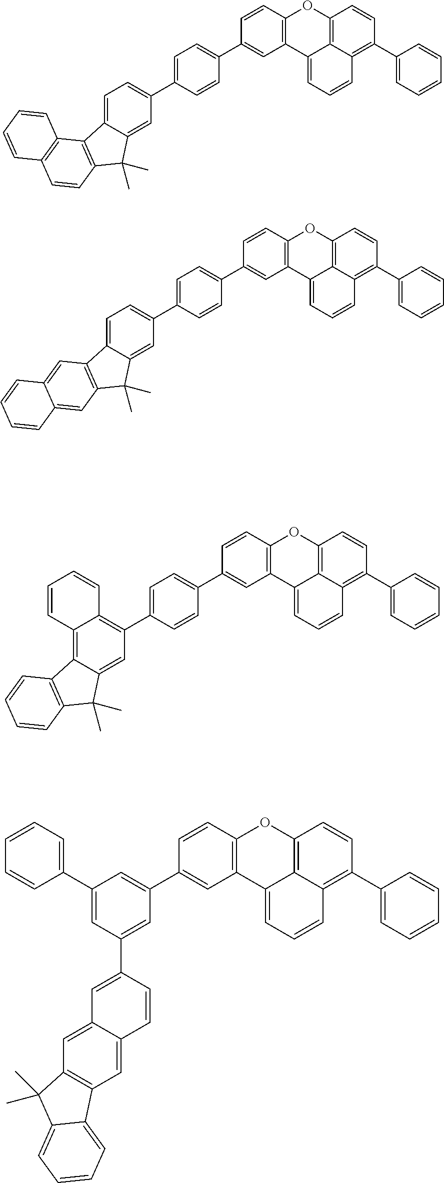 [SMILES: CC1(C)c2cc(-c3ccc(-c4ccc5c(c4)-c4cccc6c(-c7ccccc7)ccc(c46)O5)cc3)ccc2-c2c1ccc1ccccc21 ; CC1(C)c2cc(-c3ccc(-c4ccc5c(c4)-c4cccc6c(-c7ccccc7)ccc(c46)O5)cc3)ccc2-c2cc3ccccc3cc21 ; CC1(C)c2ccccc2-c2c1cc(-c1ccc(-c3ccc4c(c3)-c3cccc5c(-c6ccccc6)ccc(c35)O4)cc1)c1ccccc21 ; CC1(C)c2ccccc2-c2cc3ccc(-c4cc(-c5ccccc5)cc(-c5ccc6c(c5)-c5cccc7c(-c8ccccc8)ccc(c57)O6)c4)cc3cc21]

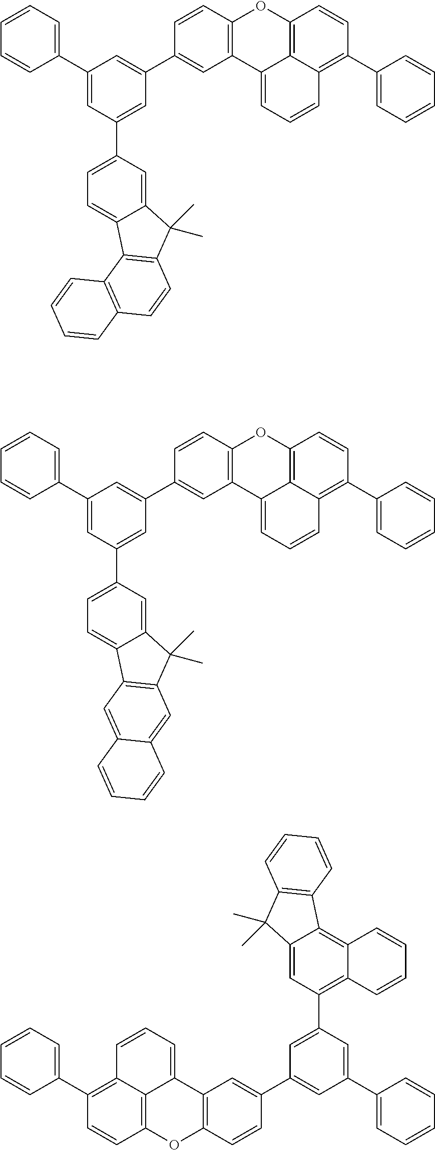 [SMILES: CC1(C)c2cc(-c3cc(-c4ccccc4)cc(-c4ccc5c(c4)-c4cccc6c(-c7ccccc7)ccc(c46)O5)c3)ccc2-c2c1ccc1ccccc21 ; CC1(C)c2cc(-c3cc(-c4ccccc4)cc(-c4ccc5c(c4)-c4cccc6c(-c7ccccc7)ccc(c46)O5)c3)ccc2-c2cc3ccccc3cc21 ; CC1(C)c2ccccc2-c2c1cc(-c1cc(-c3ccccc3)cc(-c3ccc4c(c3)-c3cccc5c(-c6ccccc6)ccc(c35)O4)c1)c1ccccc21]

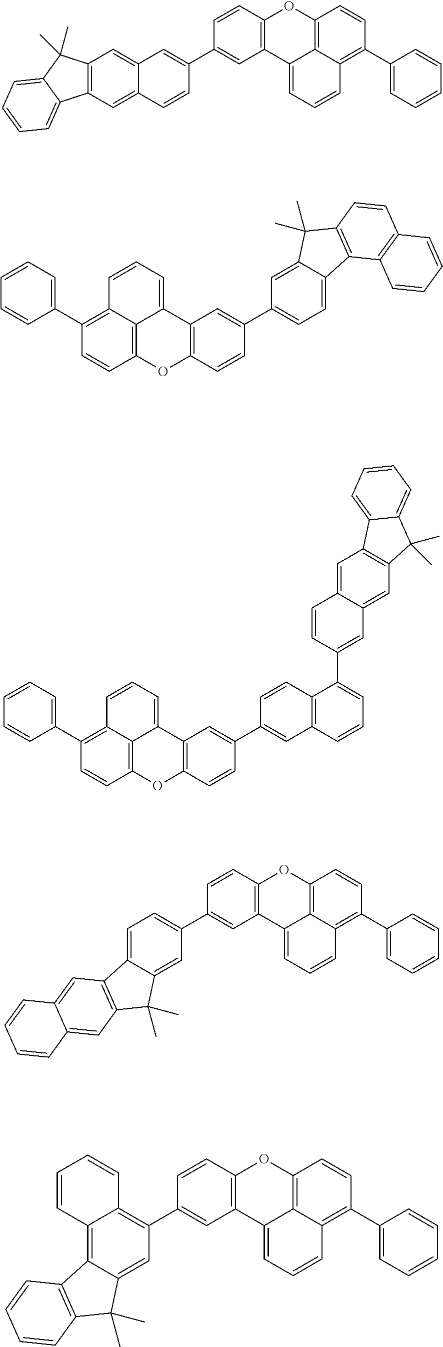 [SMILES: CC1(C)c2ccccc2-c2cc3ccc(-c4ccc5c(c4)-c4cccc6c(-c7ccccc7)ccc(c46)O5)cc3cc21 ; CC1(C)c2cc(-c3ccc4c(c3)-c3cccc5c(-c6ccccc6)ccc(c35)O4)ccc2-c2c1ccc1ccccc21 ; CC1(C)c2ccccc2-c2cc3ccc(-c4cccc5cc(-c6ccc7c(c6)-c6cccc8c(-c9ccccc9)ccc(c68)O7)ccc45)cc3cc21 ; CC1(C)c2cc(-c3ccc4c(c3)-c3cccc5c(-c6ccccc6)ccc(c35)O4)ccc2-c2cc3ccccc3cc21 ; CC1(C)c2ccccc2-c2c1cc(-c1ccc3c(c1)-c1cccc4c(-c5ccccc5)ccc(c14)O3)c1ccccc21]

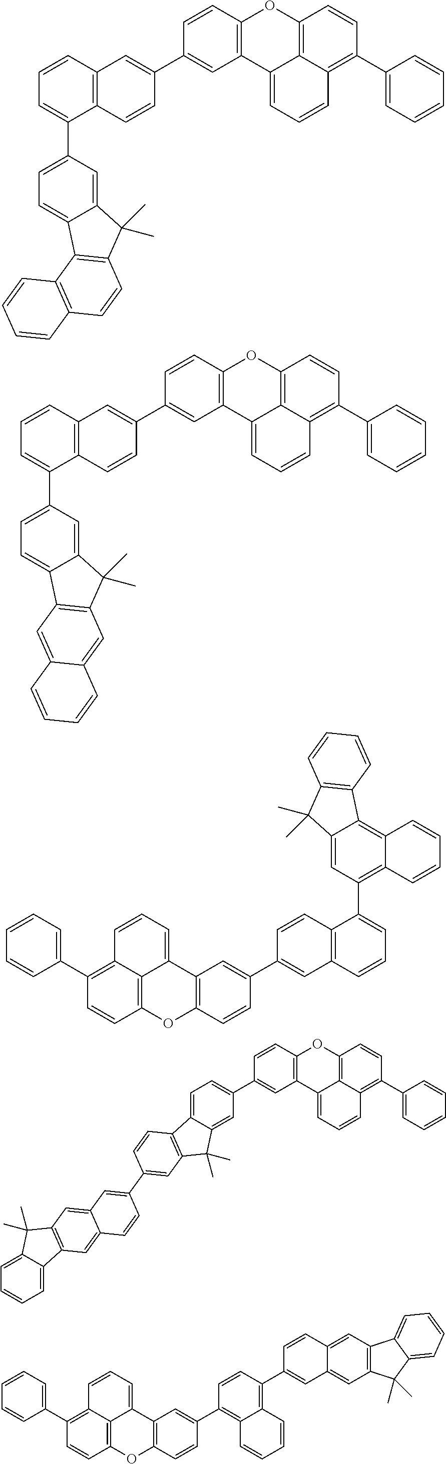 [SMILES: CC1(C)c2cc(-c3cccc4cc(-c5ccc6c(c5)-c5cccc7c(-c8ccccc8)ccc(c57)O6)ccc34)ccc2-c2c1ccc1ccccc21 ; CC1(C)c2cc(-c3cccc4cc(-c5ccc6c(c5)-c5cccc7c(-c8ccccc8)ccc(c57)O6)ccc34)ccc2-c2cc3ccccc3cc21 ; CC1(C)c2ccccc2-c2c1cc(-c1cccc3cc(-c4ccc5c(c4)-c4cccc6c(-c7ccccc7)ccc(c46)O5)ccc13)c1ccccc21 ; CC1(C)c2cc(-c3ccc4c(c3)-c3cccc5c(-c6ccccc6)ccc(c35)O4)ccc2-c2ccc(-c3ccc4cc5c(cc4c3)C(C)(C)c3ccccc3-5)cc21 ; CC1(C)c2ccccc2-c2cc3ccc(-c4ccc(-c5ccc6c(c5)-c5cccc7c(-c8ccccc8)ccc(c57)O6)c5ccccc45)cc3cc21]

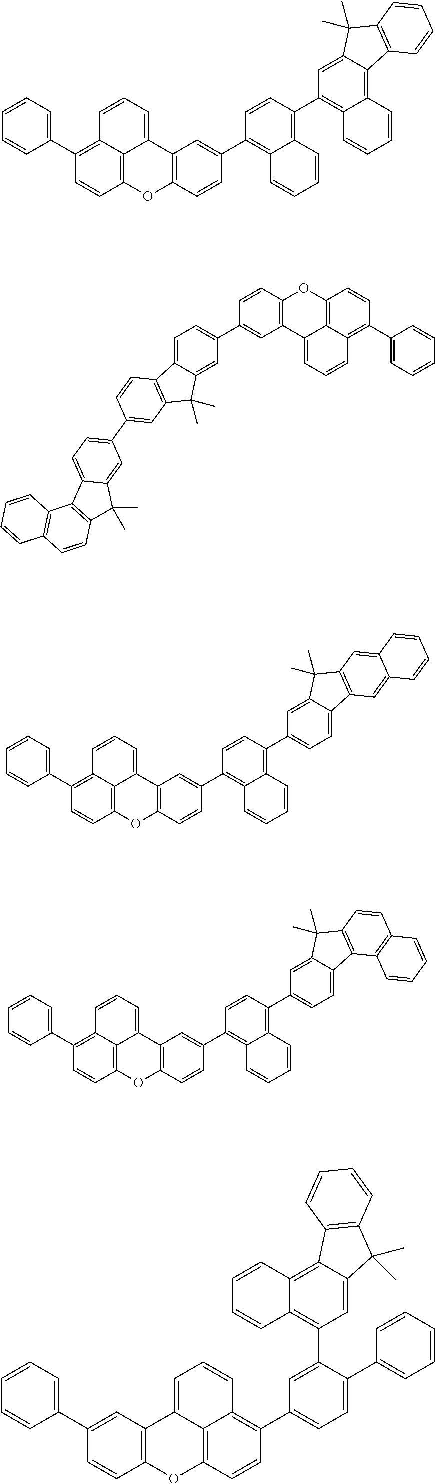 [SMILES: CC1(C)c2ccccc2-c2c1cc(-c1ccc(-c3ccc4c(c3)-c3cccc5c(-c6ccccc6)ccc(c35)O4)c3ccccc13)c1ccccc21 ; CC1(C)c2cc(-c3ccc4c(c3)-c3cccc5c(-c6ccccc6)ccc(c35)O4)ccc2-c2ccc(-c3ccc4c(c3)C(C)(C)c3ccc5ccccc5c3-4)cc21 ; CC1(C)c2cc(-c3ccc(-c4ccc5c(c4)-c4cccc6c(-c7ccccc7)ccc(c46)O5)c4ccccc34)ccc2-c2cc3ccccc3cc21 ; CC1(C)c2cc(-c3ccc(-c4ccc5c(c4)-c4cccc6c(-c7ccccc7)ccc(c46)O5)c4ccccc34)ccc2-c2c1ccc1ccccc21 ; CC1(C)c2ccccc2-c2c1cc(-c1cc(-c3ccc4c5c(cccc35)-c3cc(-c5ccccc5)ccc3O4)ccc1-c1ccccc1)c1ccccc21]

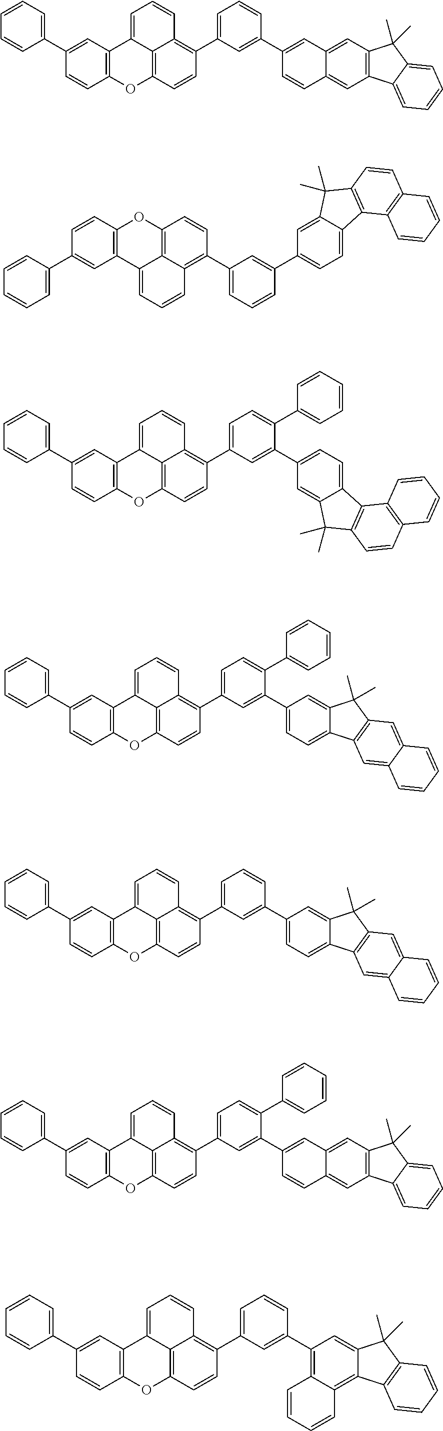

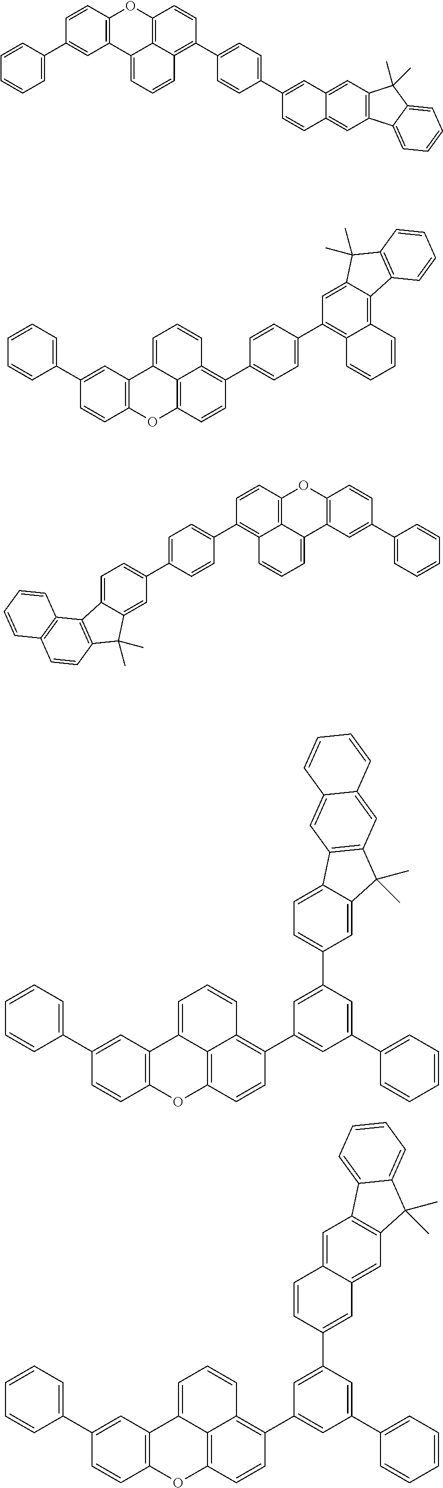

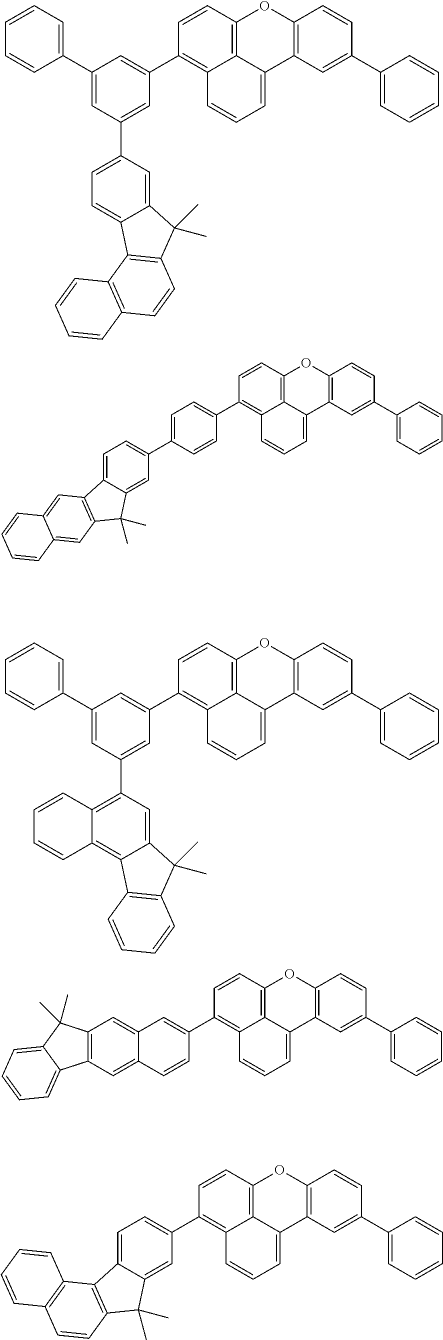 [SMILES: CC1(C)c2cc(-c3cc(-c4ccccc4)cc(-c4ccc5c6c(cccc46)-c4cc(-c6ccccc6)ccc4O5)c3)ccc2-c2c1ccc1ccccc21 ; CC1(C)c2cc(-c3ccc(-c4ccc5c6c(cccc46)-c4cc(-c6ccccc6)ccc4O5)cc3)ccc2-c2cc3ccccc3cc21 ; CC1(C)c2ccccc2-c2c1cc(-c1cc(-c3ccccc3)cc(-c3ccc4c5c(cccc35)-c3cc(-c5ccccc5)ccc3O4)c1)c1ccccc21 ; CC1(C)c2ccccc2-c2cc3ccc(-c4ccc5c6c(cccc46)-c4cc(-c6ccccc6)ccc4O5)cc3cc21 ; CC1(C)c2cc(-c3ccc4c5c(cccc35)-c3cc(-c5ccccc5)ccc3O4)ccc2-c2c1ccc1ccccc21]

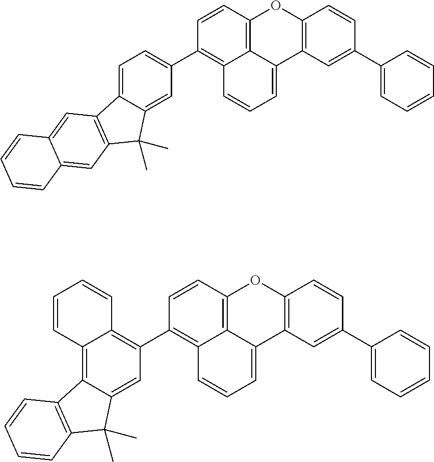 [SMILES: CC1(C)c2cc(-c3ccc4c5c(cccc35)-c3cc(-c5ccccc5)ccc3O4)ccc2-c2cc3ccccc3cc21 ; CC1(C)c2ccccc2-c2c1cc(-c1ccc3c4c(cccc14)-c1cc(-c4ccccc4)ccc1O3)c1ccccc21]

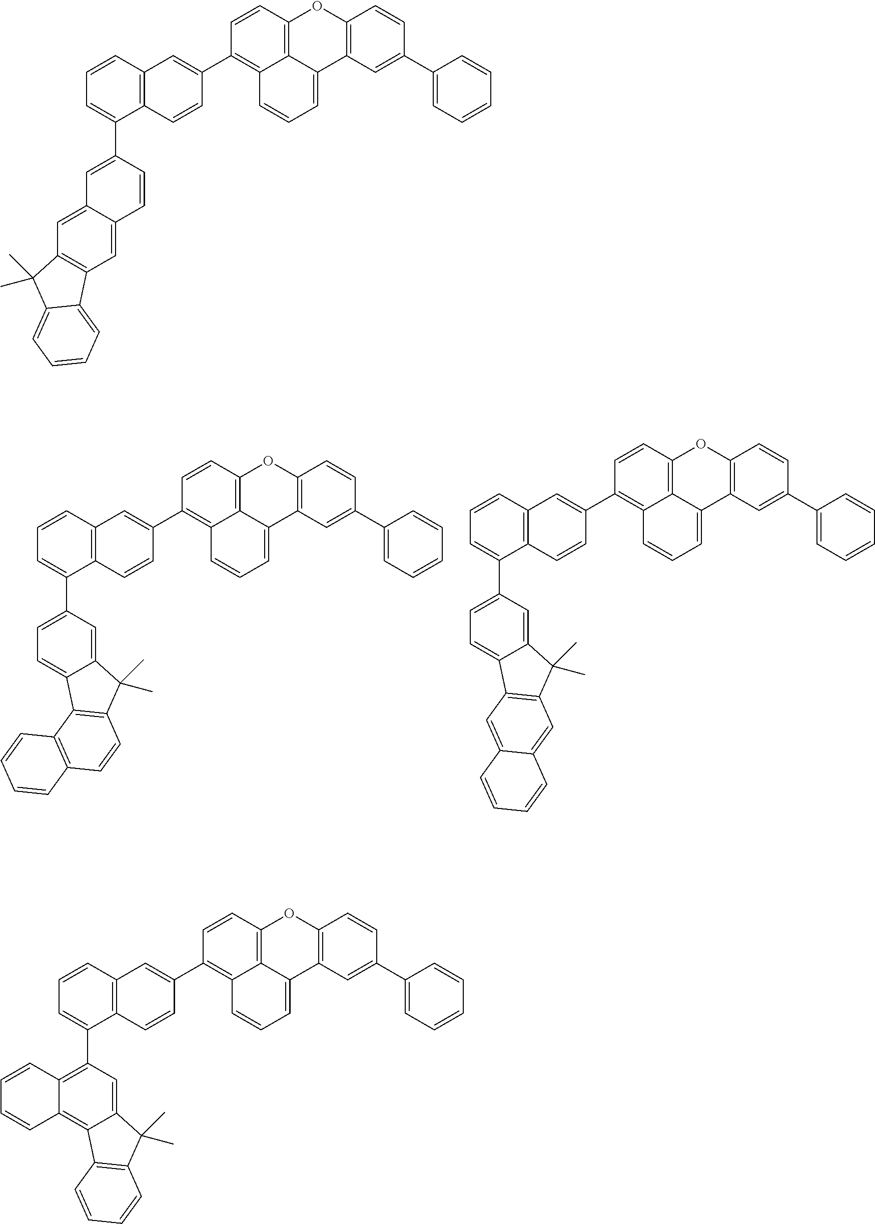

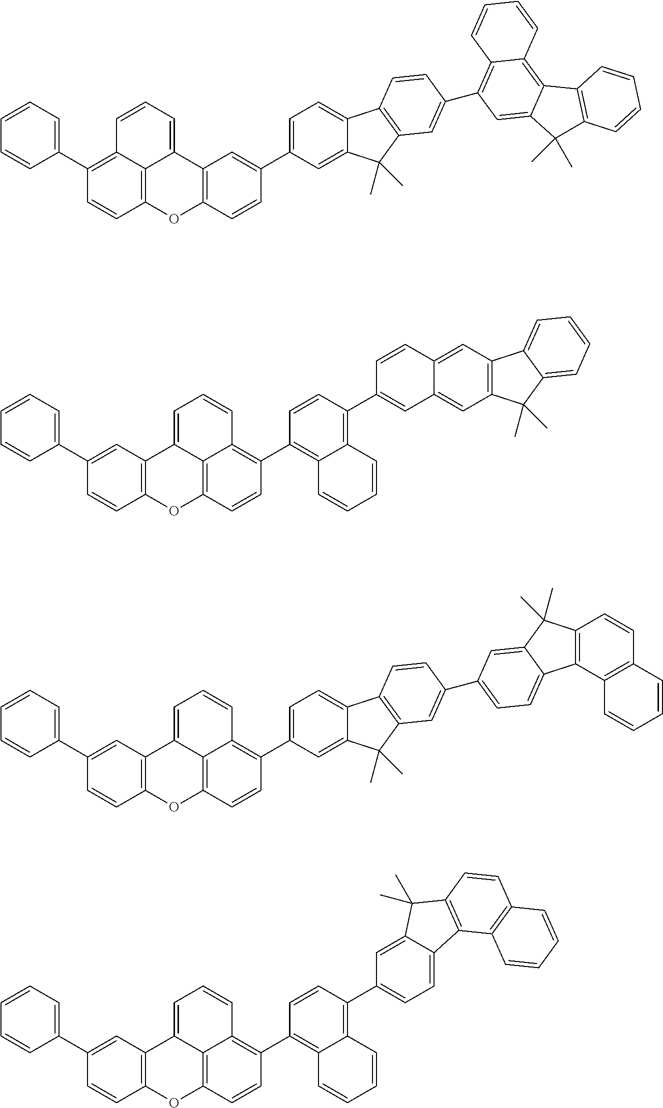 [SMILES: CC1(C)c2cc(-c3ccc4c(c3)-c3cccc5c(-c6ccccc6)ccc(c35)O4)ccc2-c2ccc(-c3cc4c(c5ccccc35)-c3ccccc3C4(C)C)cc21 ; CC1(C)c2ccccc2-c2cc3ccc(-c4ccc(-c5ccc6c7c(cccc57)-c5cc(-c7ccccc7)ccc5O6)c5ccccc45)cc3cc21 ; CC1(C)c2cc(-c3ccc4c(c3)C(C)(C)c3ccc5ccccc5c3-4)ccc2-c2ccc(-c3ccc4c5c(cccc35)-c3cc(-c5ccccc5)ccc3O4)cc21 ; CC1(C)c2cc(-c3ccc(-c4ccc5c6c(cccc46)-c4cc(-c6ccccc6)ccc4O5)c4ccccc34)ccc2-c2c1ccc1ccccc21]

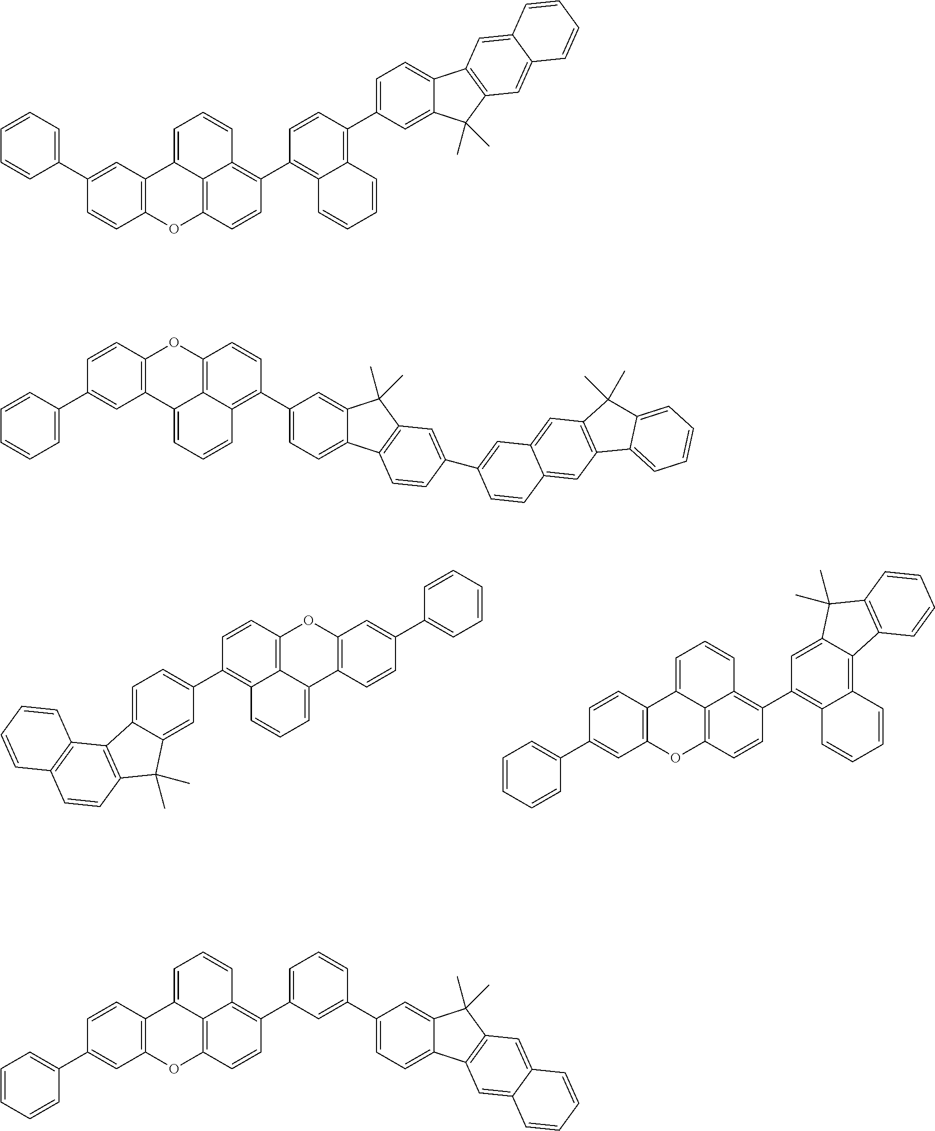 [SMILES: CC1(C)c2cc(-c3ccc(-c4ccc5c6c(cccc46)-c4cc(-c6ccccc6)ccc4O5)c4ccccc34)ccc2-c2cc3ccccc3cc21 ; CC1(C)c2cc(-c3ccc4cc5c(cc4c3)C(C)(C)c3ccccc3-5)ccc2-c2ccc(-c3ccc4c5c(cccc35)-c3cc(-c5ccccc5)ccc3O4)cc21 ; CC1(C)c2cc(-c3ccc4c5c(cccc35)-c3ccc(-c5ccccc5)cc3O4)ccc2-c2c1ccc1ccccc21 ; CC1(C)c2ccccc2-c2c1cc(-c1ccc3c4c(cccc14)-c1ccc(-c4ccccc4)cc1O3)c1ccccc21 ; CC1(C)c2cc(-c3cccc(-c4ccc5c6c(cccc46)-c4ccc(-c6ccccc6)cc4O5)c3)ccc2-c2cc3ccccc3cc21]

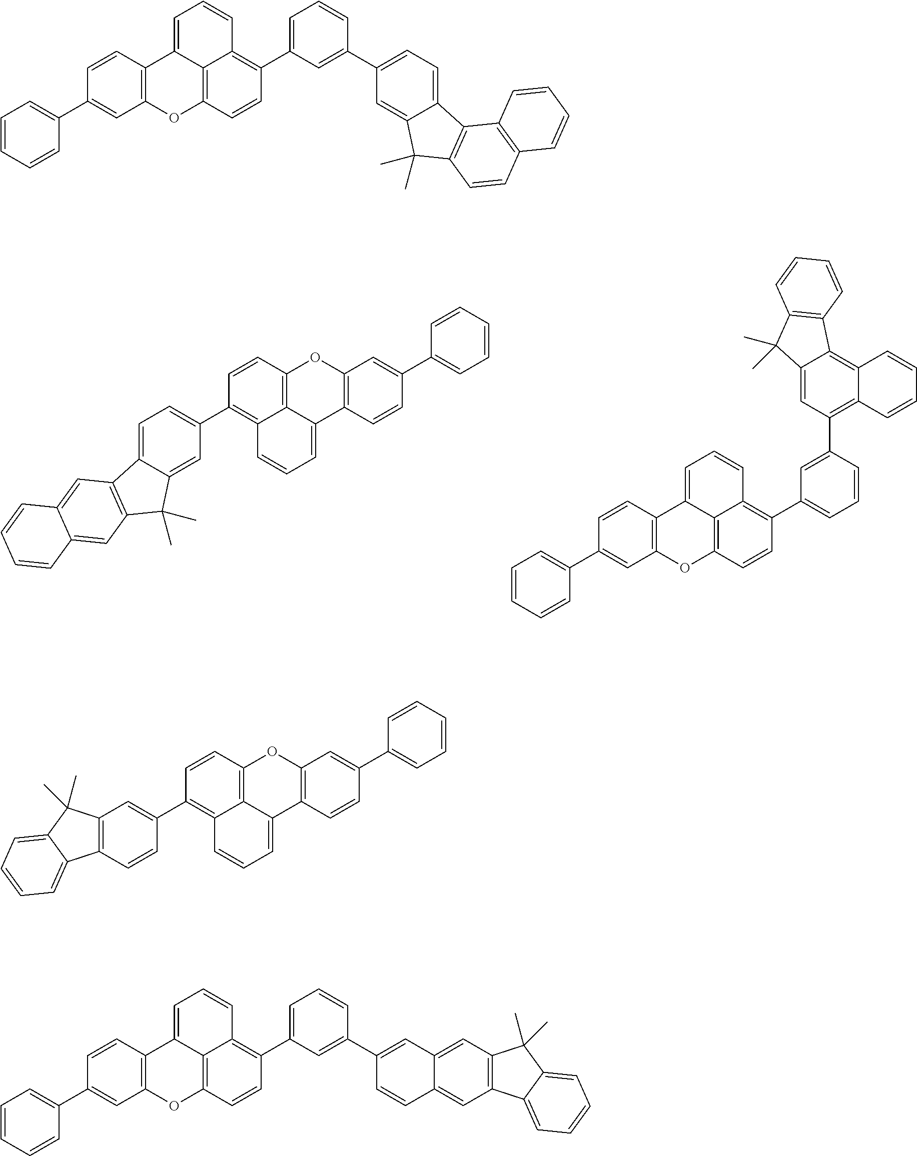 [SMILES: CC1(C)c2cc(-c3cccc(-c4ccc5c6c(cccc46)-c4ccc(-c6ccccc6)cc4O5)c3)ccc2-c2c1ccc1ccccc21 ; CC1(C)c2cc(-c3ccc4c5c(cccc35)-c3ccc(-c5ccccc5)cc3O4)ccc2-c2cc3ccccc3cc21 ; CC1(C)c2ccccc2-c2c1cc(-c1cccc(-c3ccc4c5c(cccc35)-c3ccc(-c5ccccc5)cc3O4)c1)c1ccccc21 ; CC1(C)c2ccccc2-c2ccc(-c3ccc4c5c(cccc35)-c3ccc(-c5ccccc5)cc3O4)cc21 ; CC1(C)c2ccccc2-c2cc3ccc(-c4cccc(-c5ccc6c7c(cccc57)-c5ccc(-c7ccccc7)cc5O6)c4)cc3cc21]

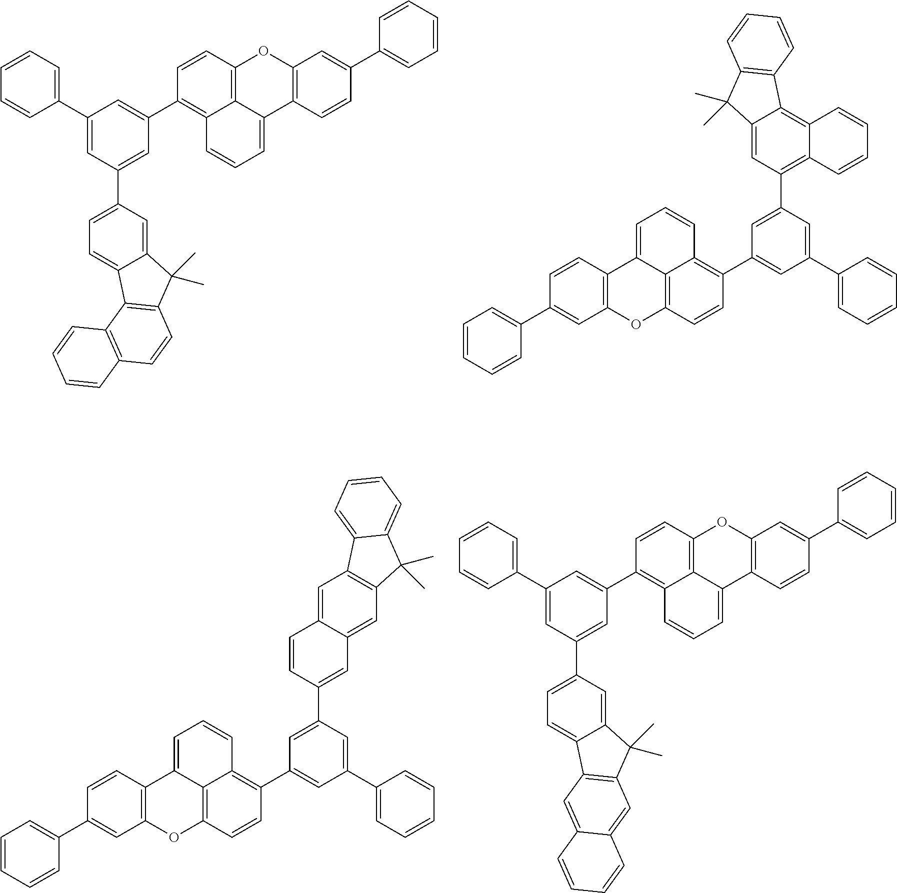

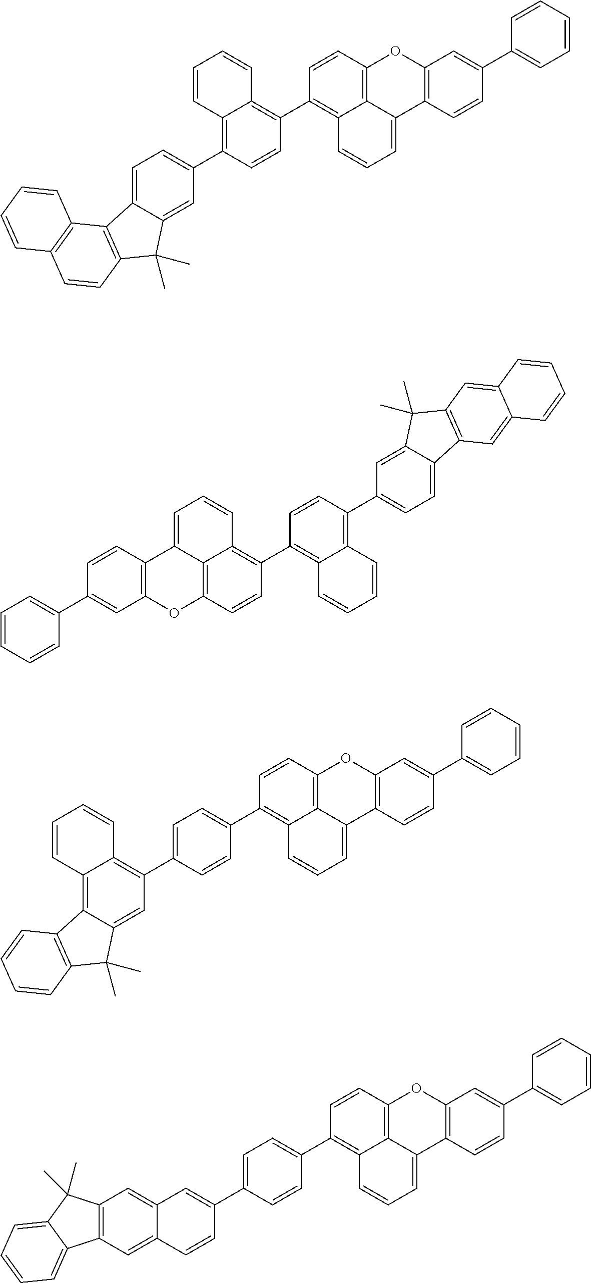 [SMILES: CC1(C)c2cc(-c3ccc(-c4ccc5c6c(cccc46)-c4ccc(-c6ccccc6)cc4O5)c4ccccc34)ccc2-c2c1ccc1ccccc21 ; CC1(C)c2cc(-c3ccc(-c4ccc5c6c(cccc46)-c4ccc(-c6ccccc6)cc4O5)c4ccccc34)ccc2-c2cc3ccccc3cc21 ; CC1(C)c2ccccc2-c2c1cc(-c1ccc(-c3ccc4c5c(cccc35)-c3ccc(-c5ccccc5)cc3O4)cc1)c1ccccc21 ; CC1(C)c2ccccc2-c2cc3ccc(-c4ccc(-c5ccc6c7c(cccc57)-c5ccc(-c7ccccc7)cc5O6)cc4)cc3cc21]

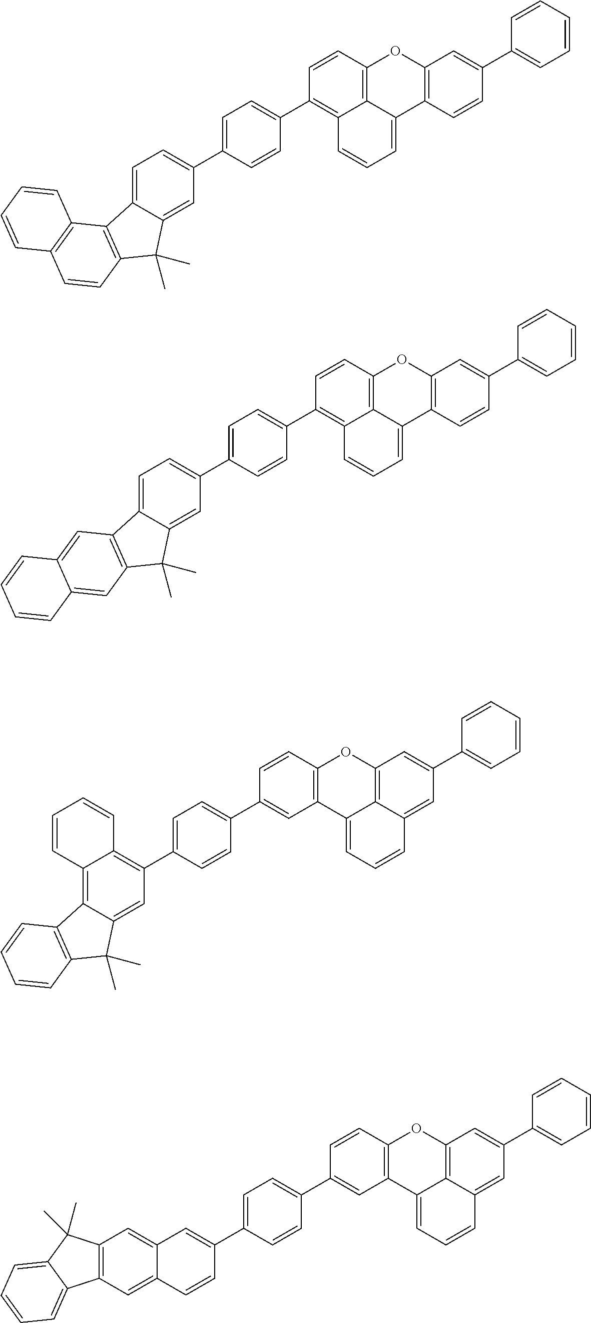 [SMILES: CC1(C)c2cc(-c3ccc(-c4ccc5c6c(cccc46)-c4ccc(-c6ccccc6)cc4O5)cc3)ccc2-c2c1ccc1ccccc21 ; CC1(C)c2cc(-c3ccc(-c4ccc5c6c(cccc46)-c4ccc(-c6ccccc6)cc4O5)cc3)ccc2-c2cc3ccccc3cc21 ; CC1(C)c2ccccc2-c2c1cc(-c1ccc(-c3ccc4c(c3)-c3cccc5cc(-c6ccccc6)cc(c35)O4)cc1)c1ccccc21 ; CC1(C)c2ccccc2-c2cc3ccc(-c4ccc(-c5ccc6c(c5)-c5cccc7cc(-c8ccccc8)cc(c57)O6)cc4)cc3cc21]

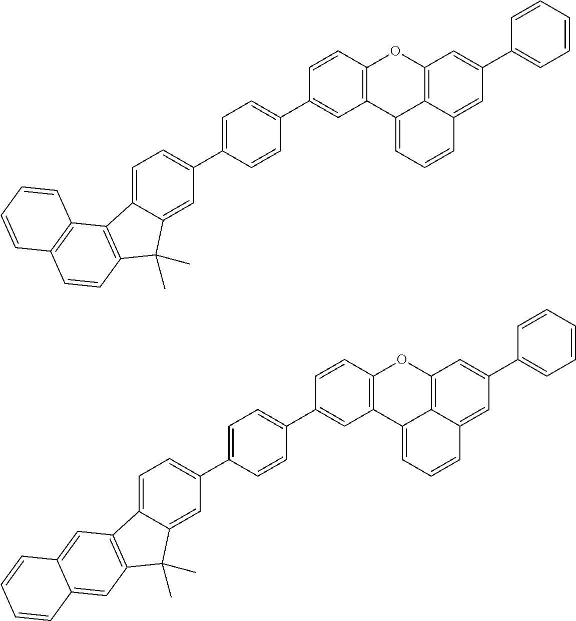 [SMILES: CC1(C)c2cc(-c3ccc(-c4ccc5c(c4)-c4cccc6cc(-c7ccccc7)cc(c46)O5)cc3)ccc2-c2c1ccc1ccccc21 ; CC1(C)c2cc(-c3ccc(-c4ccc5c(c4)-c4cccc6cc(-c7ccccc7)cc(c46)O5)cc3)ccc2-c2cc3ccccc3cc21]

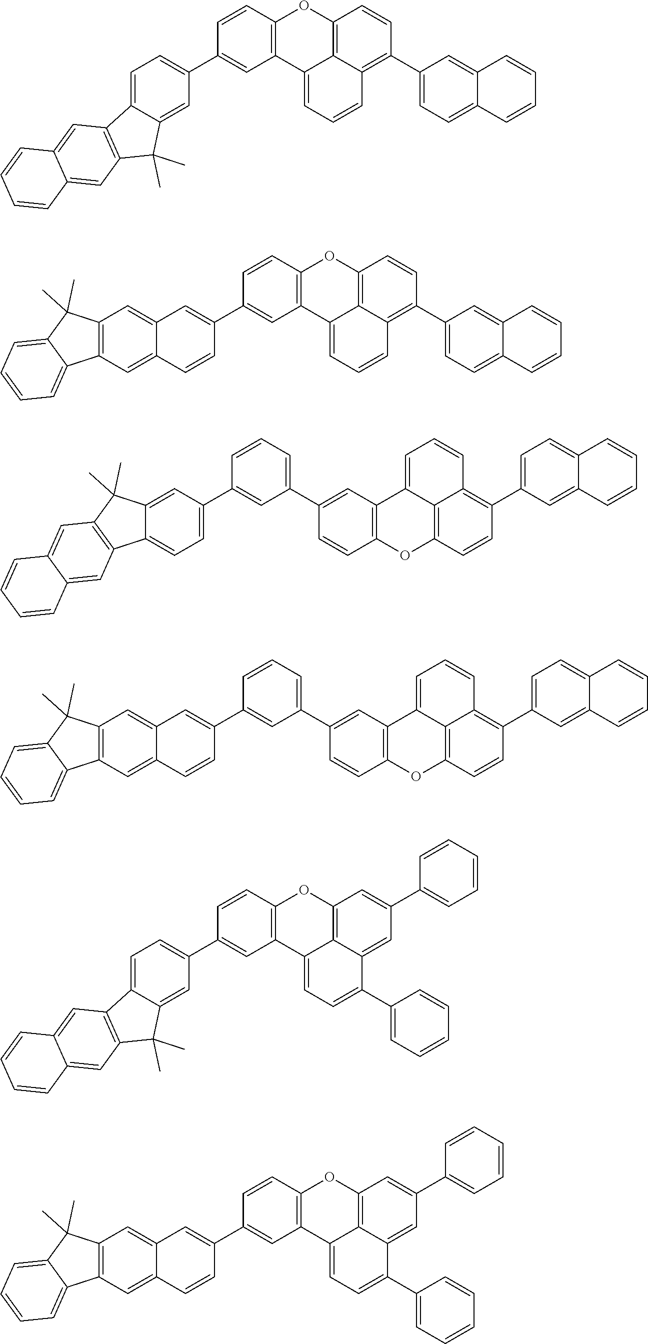

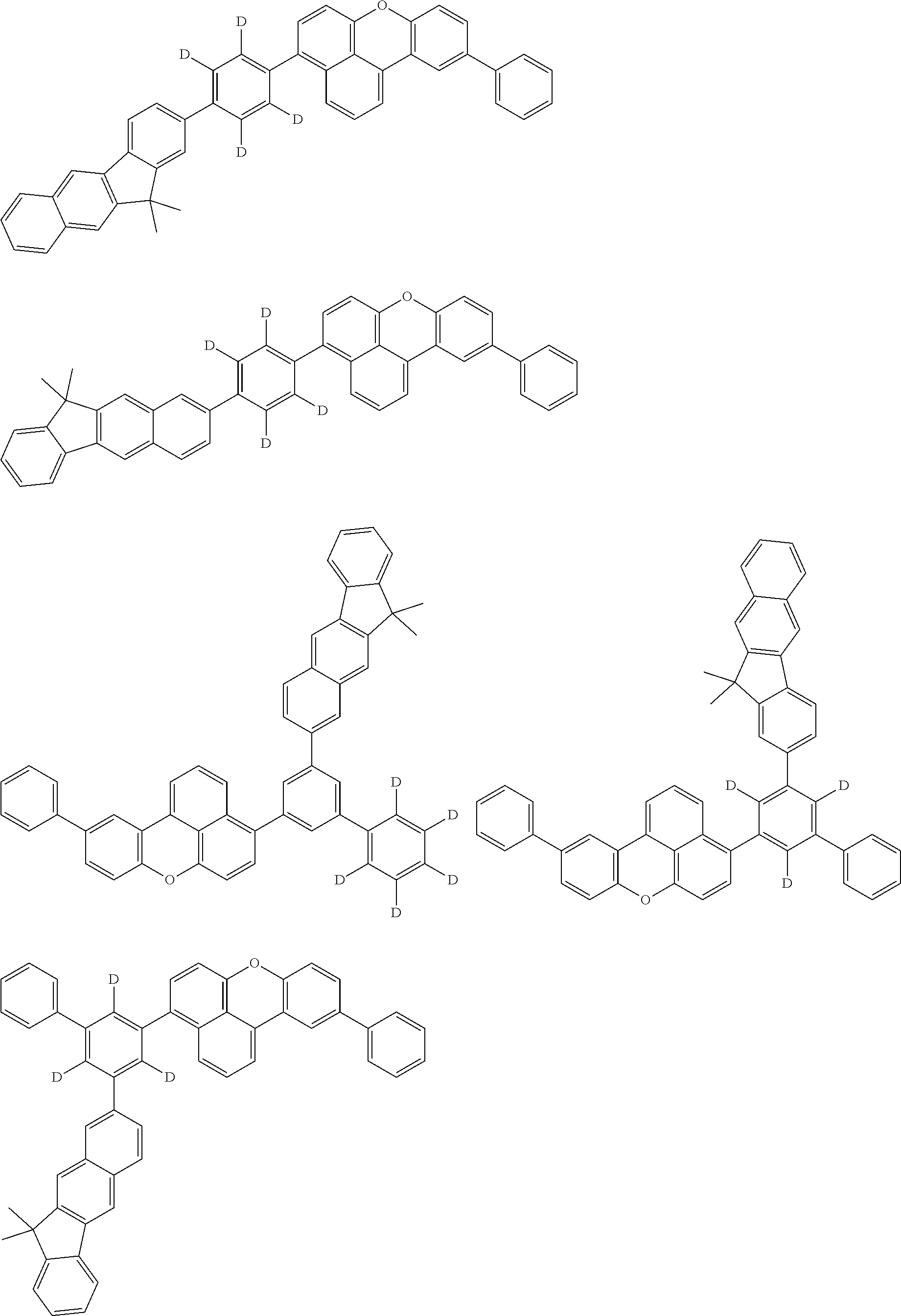

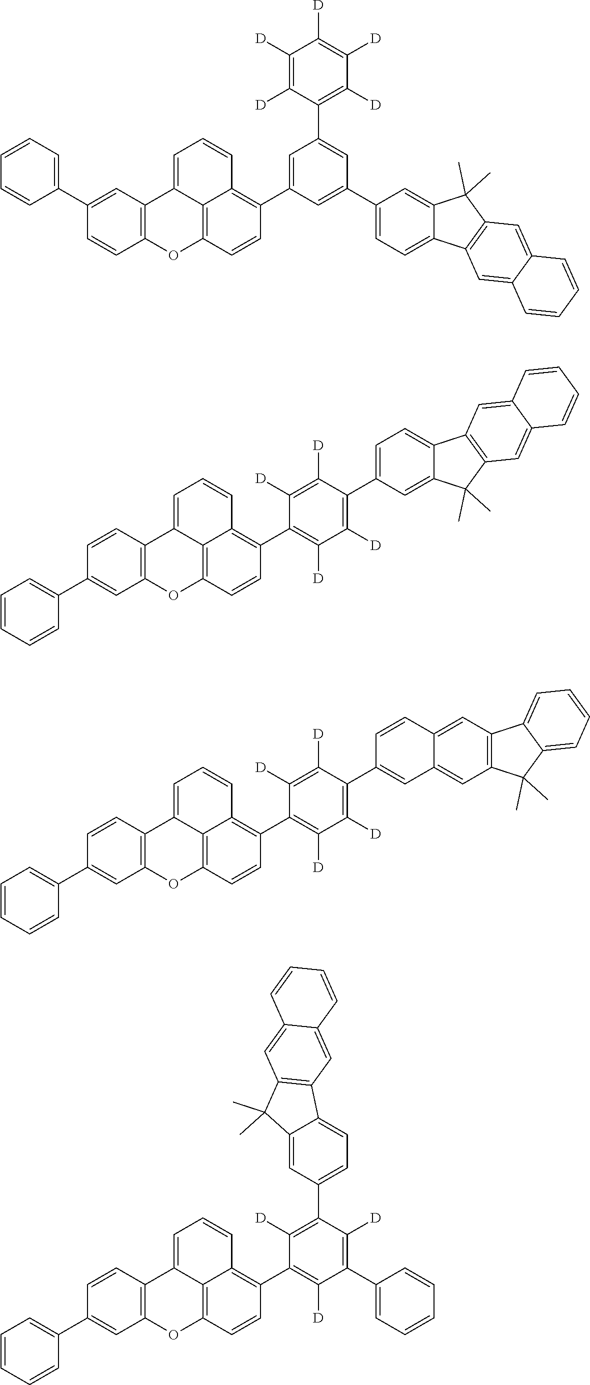 [SMILES: [2H]c1c([2H])c([2H])c(-c2cc(-c3ccc4c(c3)C(C)(C)c3cc5ccccc5cc3-4)cc(-c3ccc4c5c(cccc35)-c3cc(-c5ccccc5)ccc3O4)c2)c([2H])c1[2H] ; [2H]c1c([2H])c(-c2ccc3c4c(cccc24)-c2ccc(-c4ccccc4)cc2O3)c([2H])c([2H])c1-c1ccc2c(c1)C(C)(C)c1cc3ccccc3cc1-2 ; [2H]c1c([2H])c(-c2ccc3c4c(cccc24)-c2ccc(-c4ccccc4)cc2O3)c([2H])c([2H])c1-c1ccc2cc3c(cc2c1)C(C)(C)c1ccccc1-3 ; [2H]c1c(-c2ccccc2)c([2H])c(-c2ccc3c4c(cccc24)-c2ccc(-c4ccccc4)cc2O3)c([2H])c1-c1ccc2c(c1)C(C)(C)c1cc3ccccc3cc1-2]

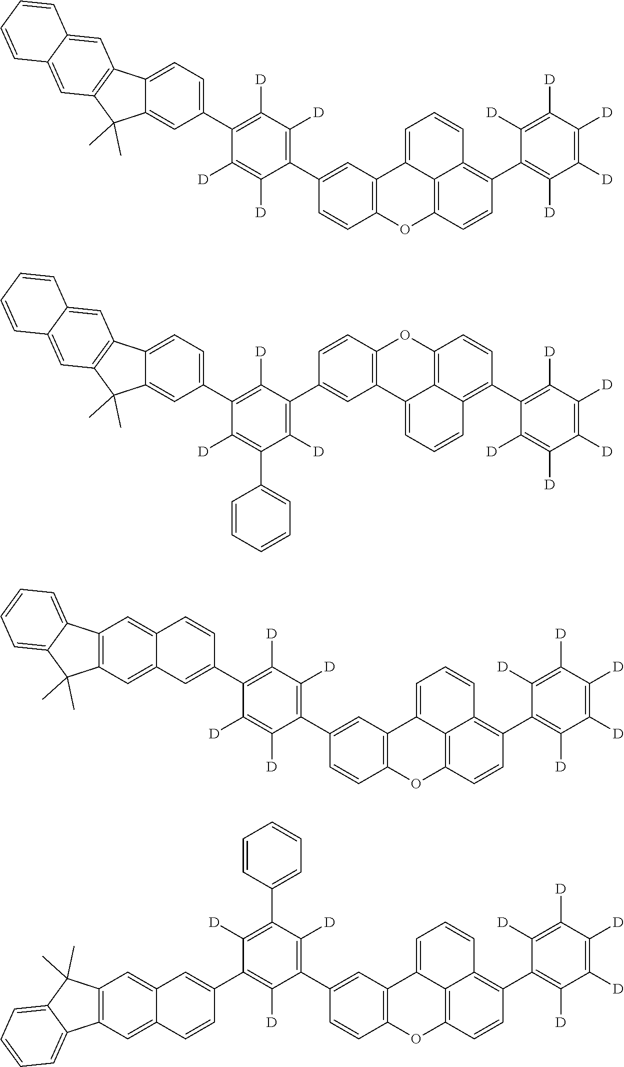

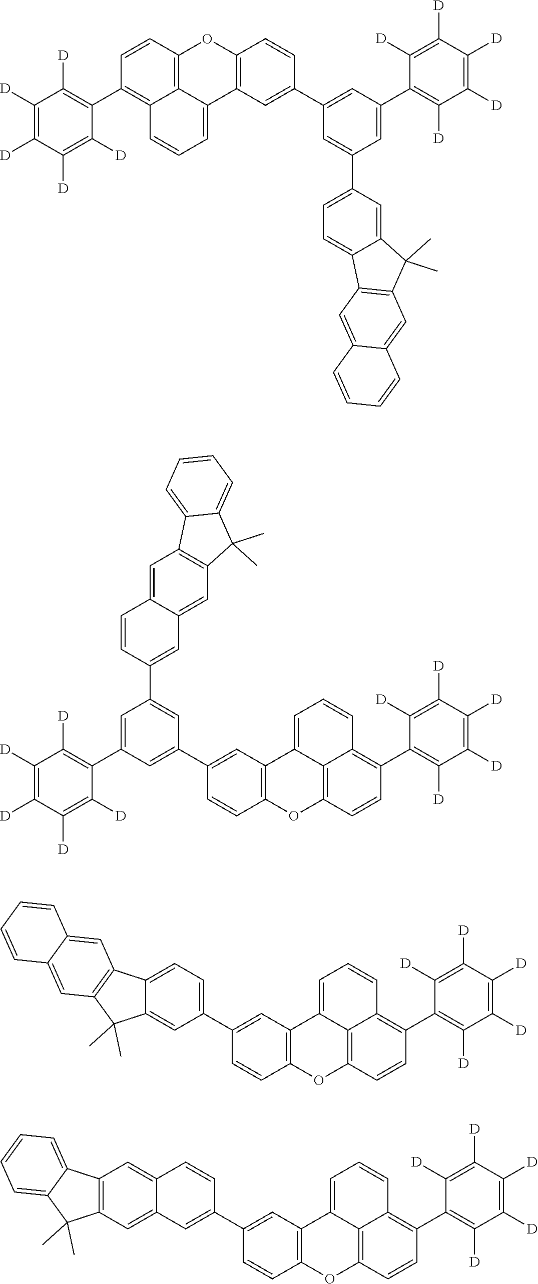

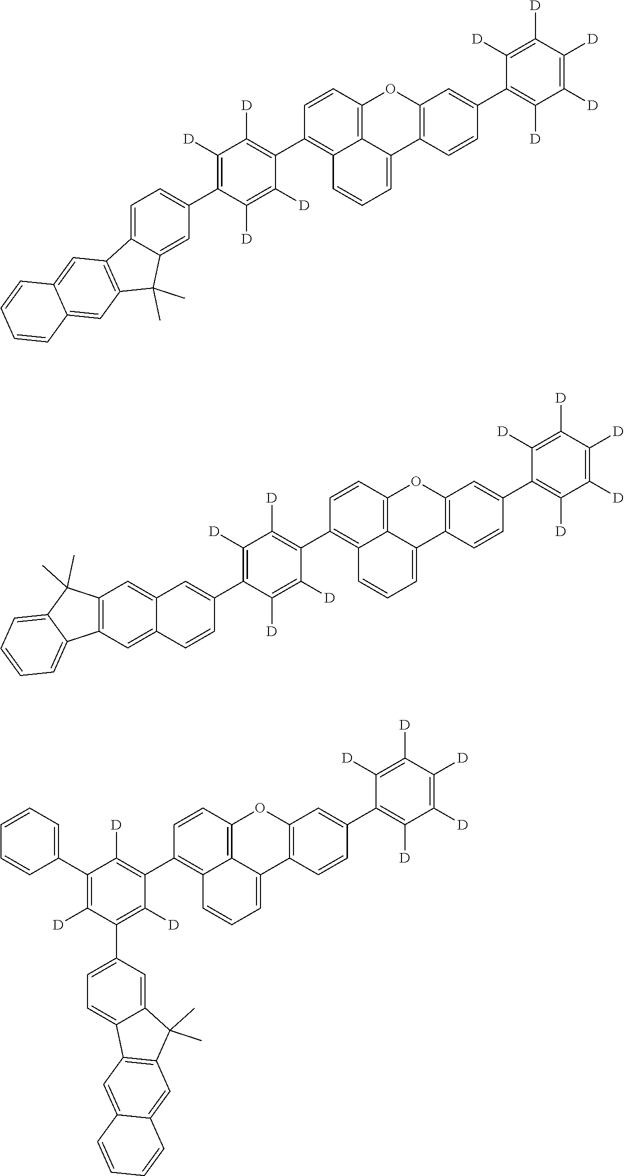 [SMILES: [2H]c1c([2H])c([2H])c(-c2ccc3c(c2)Oc2ccc(-c4c([2H])c([2H])c(-c5ccc6c(c5)C(C)(C)c5cc7ccccc7cc5-6)c([2H])c4[2H])c4cccc-3c24)c([2H])c1[2H] ; [2H]c1c([2H])c([2H])c(-c2ccc3c(c2)Oc2ccc(-c4c([2H])c([2H])c(-c5ccc6cc7c(cc6c5)C(C)(C)c5ccccc5-7)c([2H])c4[2H])c4cccc-3c24)c([2H])c1[2H] ; [2H]c1c([2H])c([2H])c(-c2ccc3c(c2)Oc2ccc(-c4c([2H])c(-c5ccccc5)c([2H])c(-c5ccc6c(c5)C(C)(C)c5cc7ccccc7cc5-6)c4[2H])c4cccc-3c24)c([2H])c1[2H]]

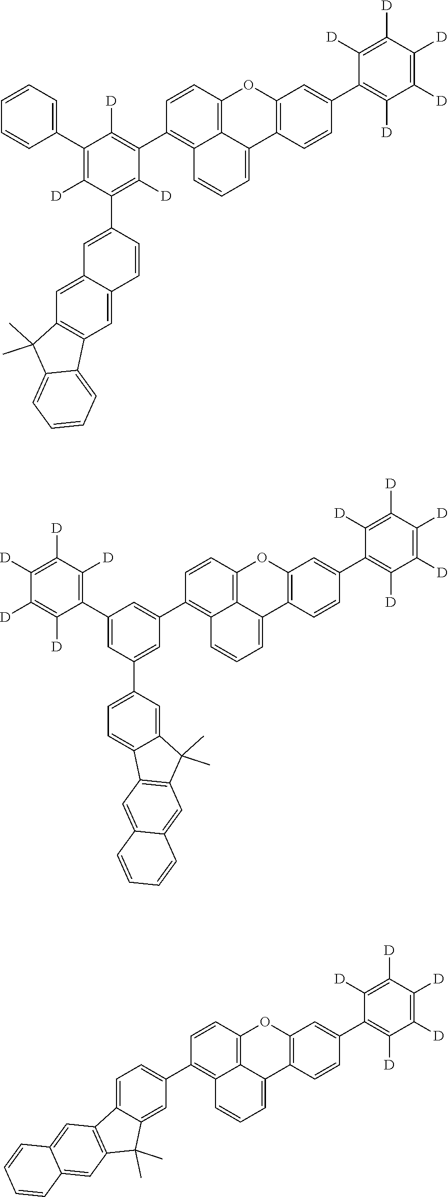 [SMILES: [2H]c1c([2H])c([2H])c(-c2ccc3c(c2)Oc2ccc(-c4c([2H])c(-c5ccccc5)c([2H])c(-c5ccc6cc7c(cc6c5)C(C)(C)c5ccccc5-7)c4[2H])c4cccc-3c24)c([2H])c1[2H] ; [2H]c1c([2H])c([2H])c(-c2cc(-c3ccc4c(c3)C(C)(C)c3cc5ccccc5cc3-4)cc(-c3ccc4c5c(cccc35)-c3ccc(-c5c([2H])c([2H])c([2H])c([2H])c5[2H])cc3O4)c2)c([2H])c1[2H] ; [2H]c1c([2H])c([2H])c(-c2ccc3c(c2)Oc2ccc(-c4ccc5c(c4)C(C)(C)c4cc6ccccc6cc4-5)c4cccc-3c24)c([2H])c1[2H]]

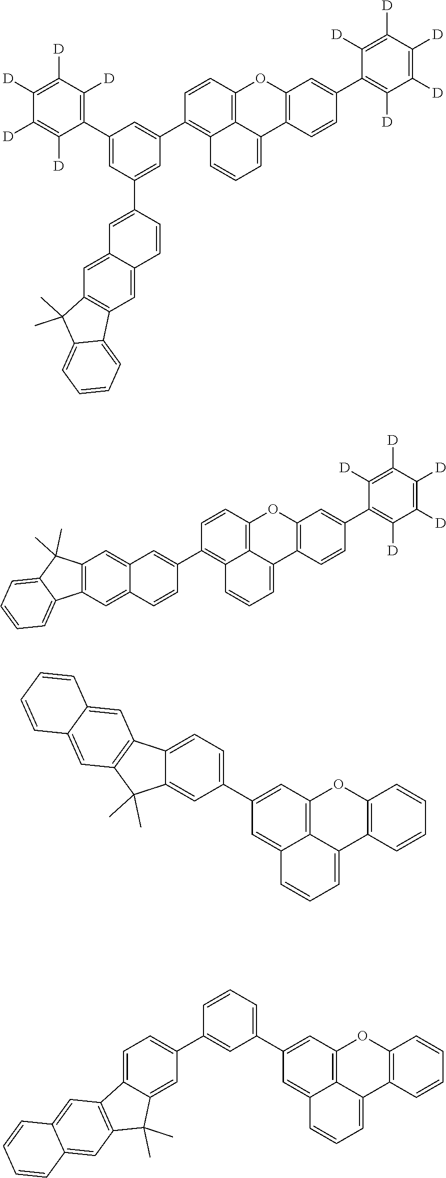 [SMILES: [2H]c1c([2H])c([2H])c(-c2cc(-c3ccc4cc5c(cc4c3)C(C)(C)c3ccccc3-5)cc(-c3ccc4c5c(cccc35)-c3ccc(-c5c([2H])c([2H])c([2H])c([2H])c5[2H])cc3O4)c2)c([2H])c1[2H] ; [2H]c1c([2H])c([2H])c(-c2ccc3c(c2)Oc2ccc(-c4ccc5cc6c(cc5c4)C(C)(C)c4ccccc4-6)c4cccc-3c24)c([2H])c1[2H] ; CC1(C)c2cc(-c3cc4c5c(cccc5c3)-c3ccccc3O4)ccc2-c2cc3ccccc3cc21 ; CC1(C)c2cc(-c3cccc(-c4cc5c6c(cccc6c4)-c4ccccc4O5)c3)ccc2-c2cc3ccccc3cc21]

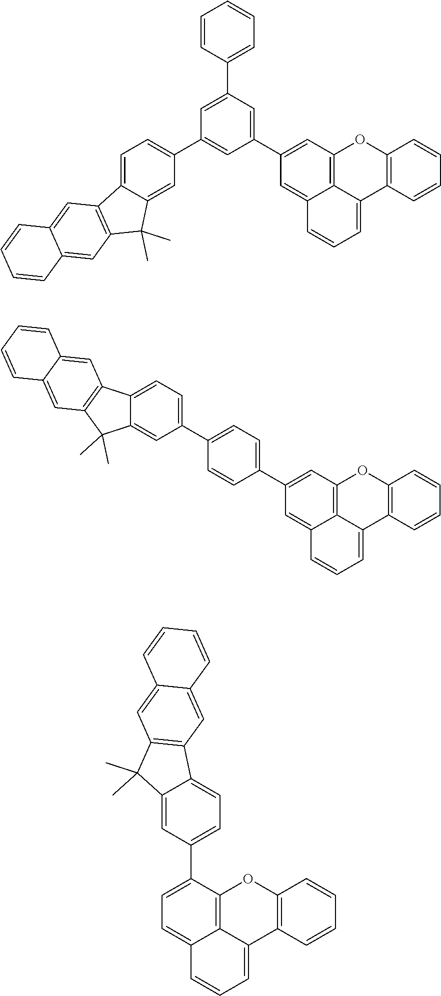 [SMILES: CC1(C)c2cc(-c3cc(-c4ccccc4)cc(-c4cc5c6c(cccc6c4)-c4ccccc4O5)c3)ccc2-c2cc3ccccc3cc21 ; CC1(C)c2cc(-c3ccc(-c4cc5c6c(cccc6c4)-c4ccccc4O5)cc3)ccc2-c2cc3ccccc3cc21 ; CC1(C)c2cc(-c3ccc4cccc5c4c3Oc3ccccc3-5)ccc2-c2cc3ccccc3cc21]

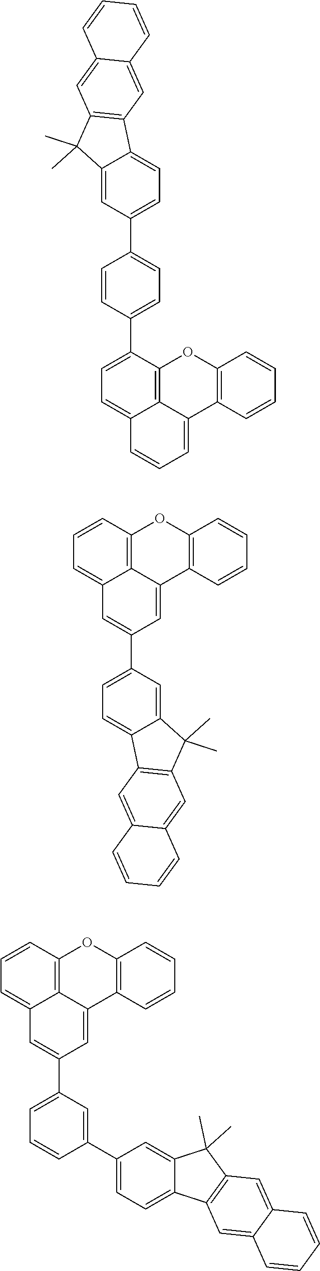 [SMILES: CC1(C)c2cc(-c3ccc(-c4ccc5cccc6c5c4Oc4ccccc4-6)cc3)ccc2-c2cc3ccccc3cc21 ; CC1(C)c2cc(-c3cc4c5c(cccc5c3)Oc3ccccc3-4)ccc2-c2cc3ccccc3cc21 ; CC1(C)c2cc(-c3cccc(-c4cc5c6c(cccc6c4)Oc4ccccc4-5)c3)ccc2-c2cc3ccccc3cc21]

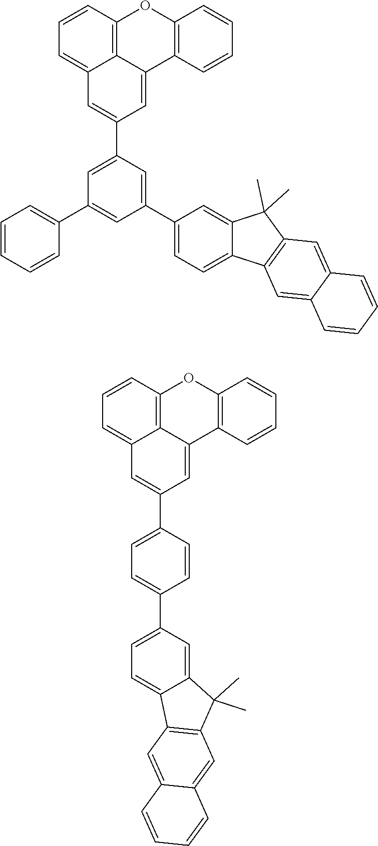 [SMILES: CC1(C)c2cc(-c3cc(-c4ccccc4)cc(-c4cc5c6c(cccc6c4)Oc4ccccc4-5)c3)ccc2-c2cc3ccccc3cc21 ; CC1(C)c2cc(-c3ccc(-c4cc5c6c(cccc6c4)Oc4ccccc4-5)cc3)ccc2-c2cc3ccccc3cc21]

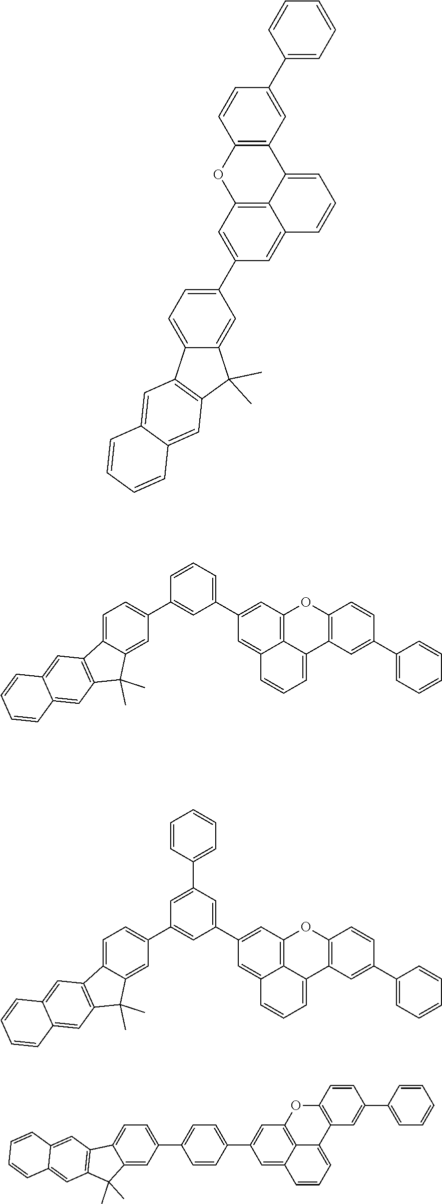 [SMILES: CC1(C)c2cc(-c3cc4c5c(cccc5c3)-c3cc(-c5ccccc5)ccc3O4)ccc2-c2cc3ccccc3cc21 ; CC1(C)c2cc(-c3cccc(-c4cc5c6c(cccc6c4)-c4cc(-c6ccccc6)ccc4O5)c3)ccc2-c2cc3ccccc3cc21 ; CC1(C)c2cc(-c3cc(-c4ccccc4)cc(-c4cc5c6c(cccc6c4)-c4cc(-c6ccccc6)ccc4O5)c3)ccc2-c2cc3ccccc3cc21 ; CC1(C)c2cc(-c3ccc(-c4cc5c6c(cccc6c4)-c4cc(-c6ccccc6)ccc4O5)cc3)ccc2-c2cc3ccccc3cc21]

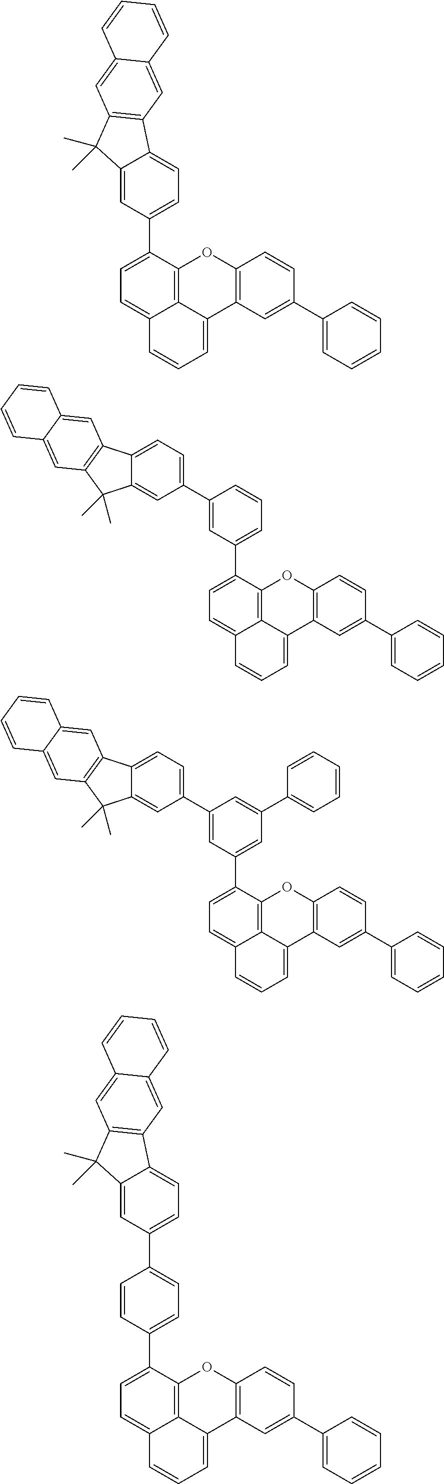 [SMILES: CC1(C)c2cc(-c3ccc4cccc5c4c3Oc3ccc(-c4ccccc4)cc3-5)ccc2-c2cc3ccccc3cc21 ; CC1(C)c2cc(-c3cccc(-c4ccc5cccc6c5c4Oc4ccc(-c5ccccc5)cc4-6)c3)ccc2-c2cc3ccccc3cc21 ; CC1(C)c2cc(-c3cc(-c4ccccc4)cc(-c4ccc5cccc6c5c4Oc4ccc(-c5ccccc5)cc4-6)c3)ccc2-c2cc3ccccc3cc21 ; CC1(C)c2cc(-c3ccc(-c4ccc5cccc6c5c4Oc4ccc(-c5ccccc5)cc4-6)cc3)ccc2-c2cc3ccccc3cc21]

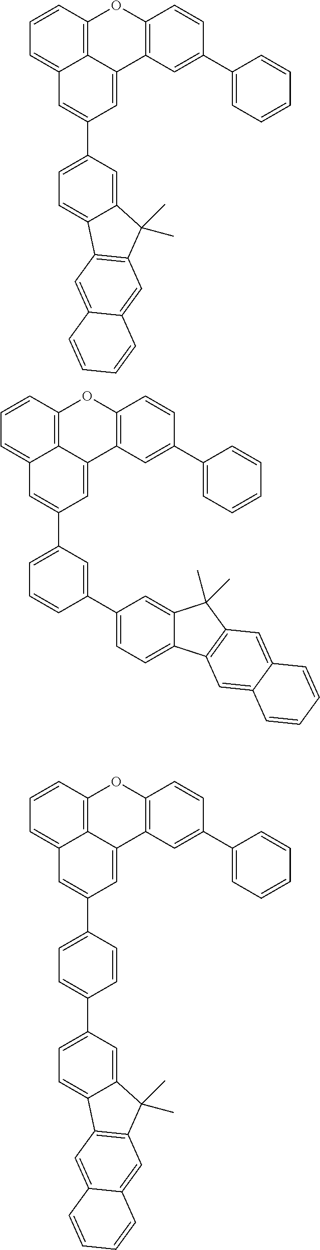

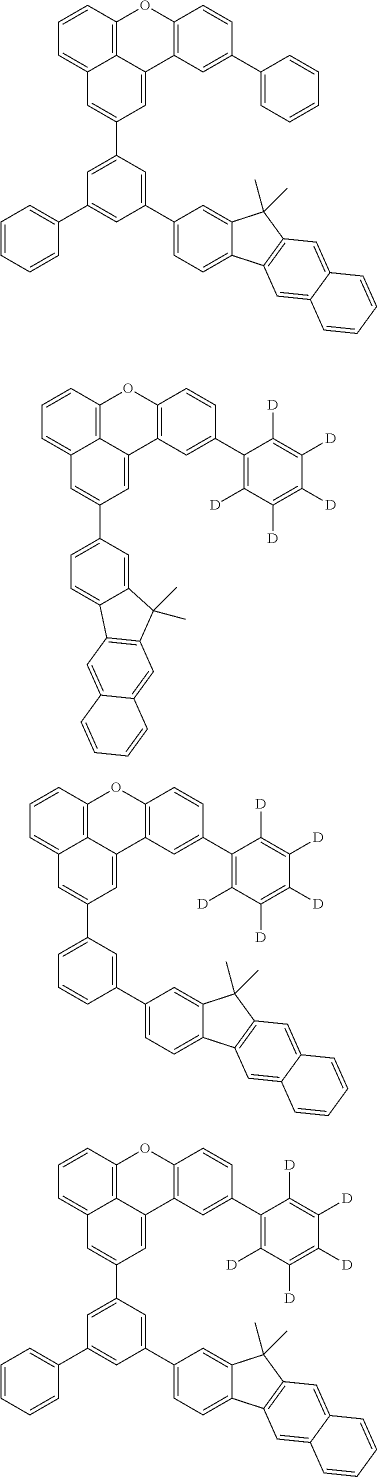

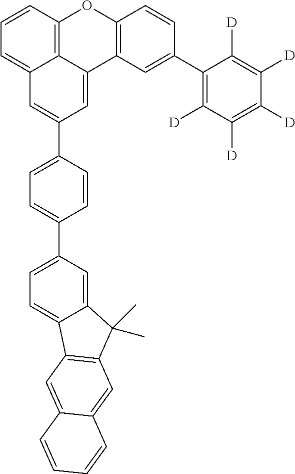 [SMILES: [2H]c1c([2H])c([2H])c(-c2ccc3c(c2)-c2cc(-c4ccc(-c5ccc6c(c5)C(C)(C)c5cc7ccccc7cc5-6)cc4)cc4cccc(c24)O3)c([2H])c1[2H]]

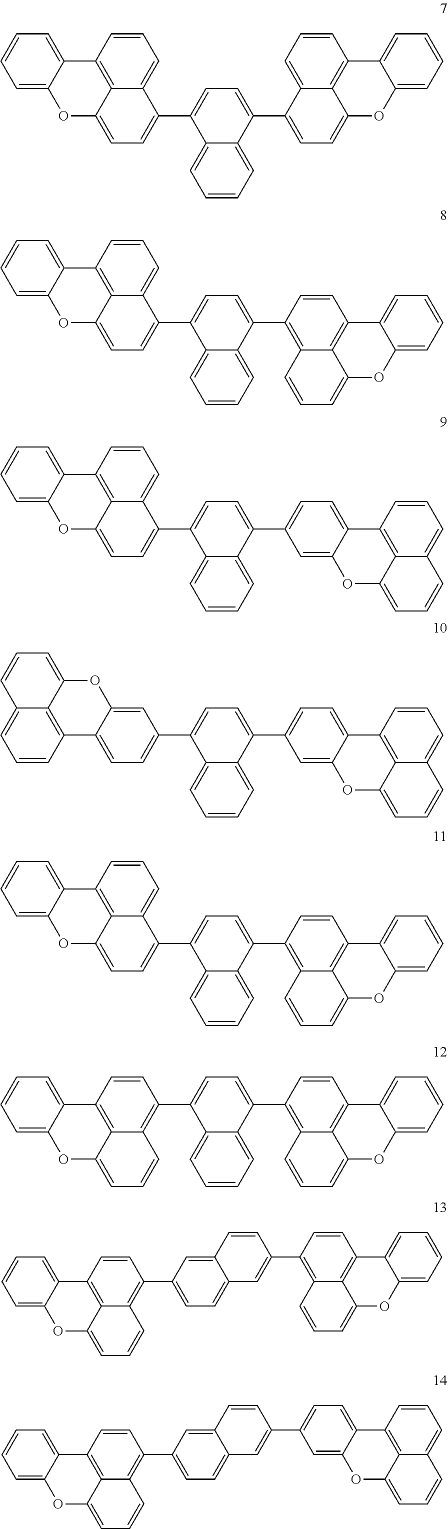

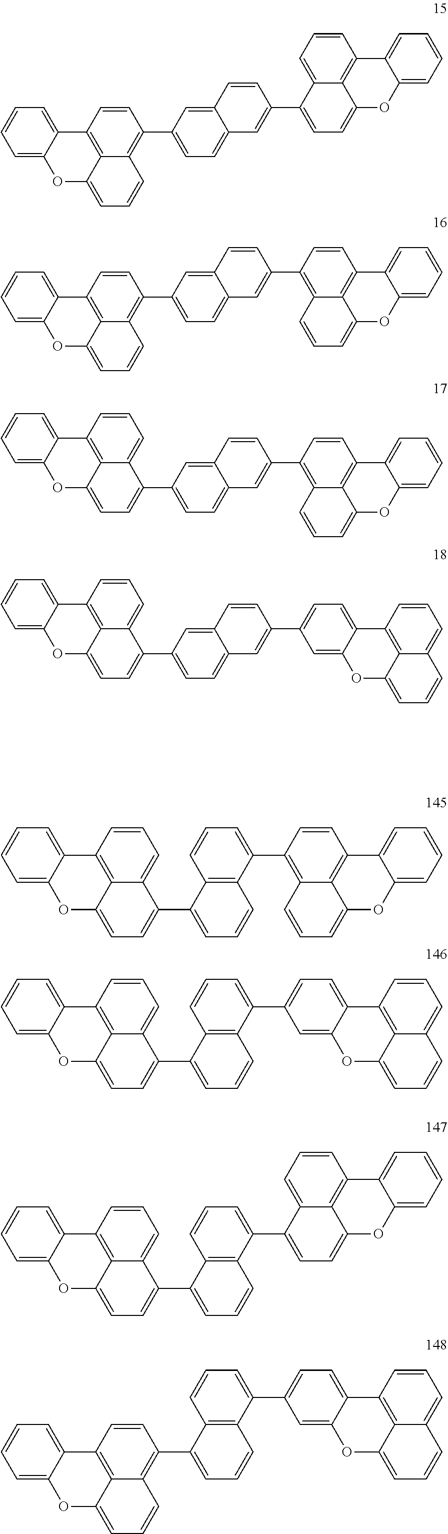

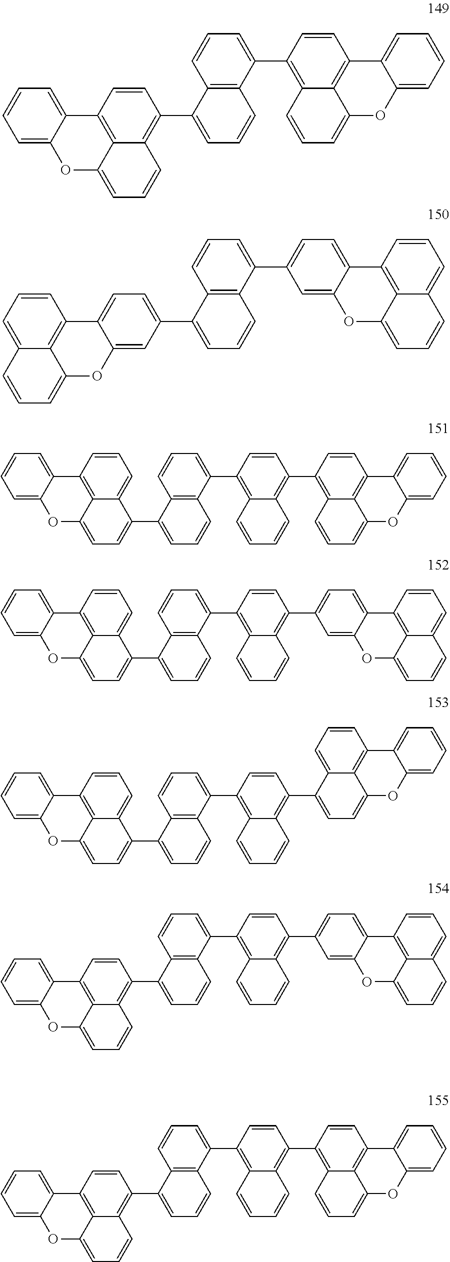

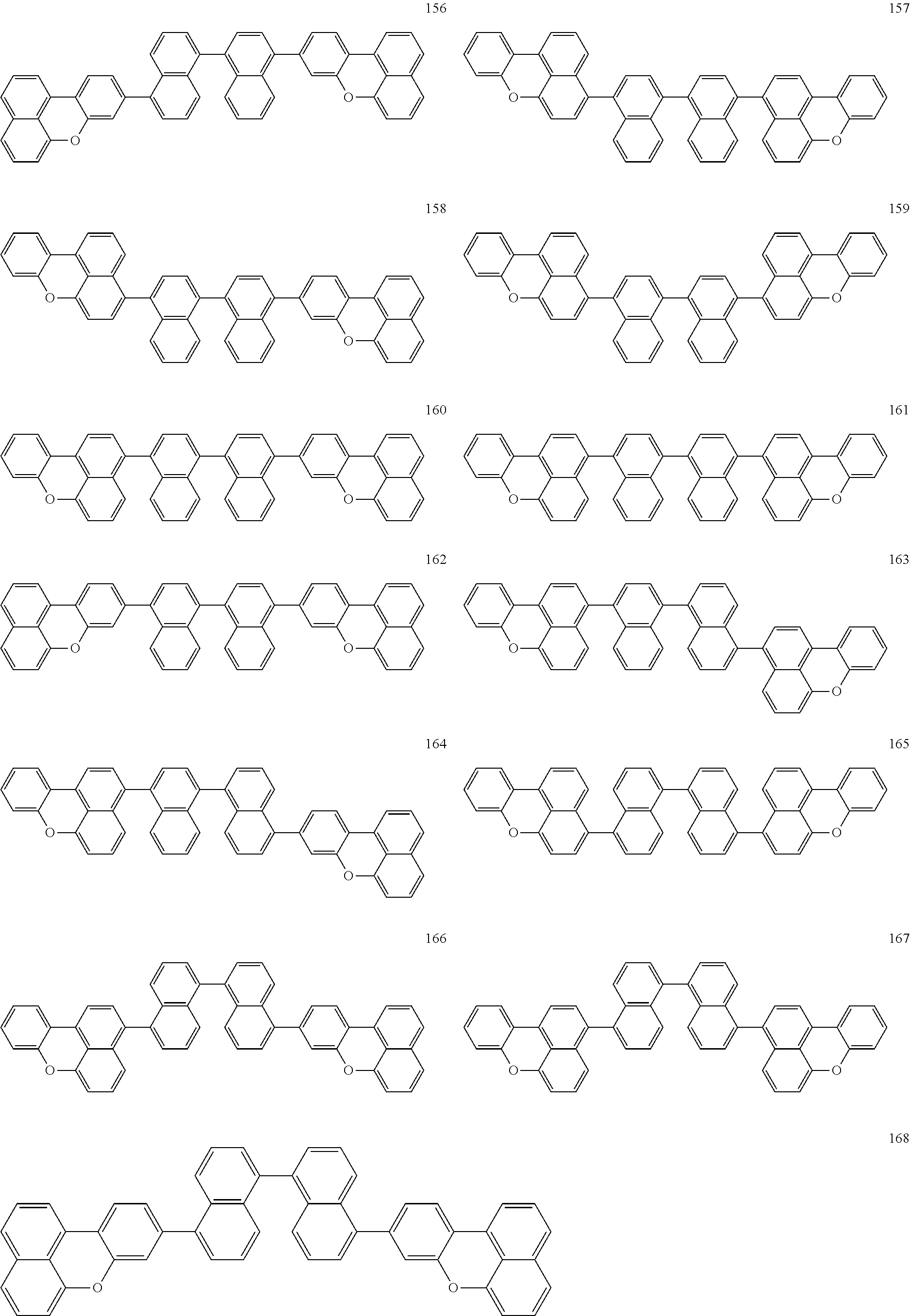

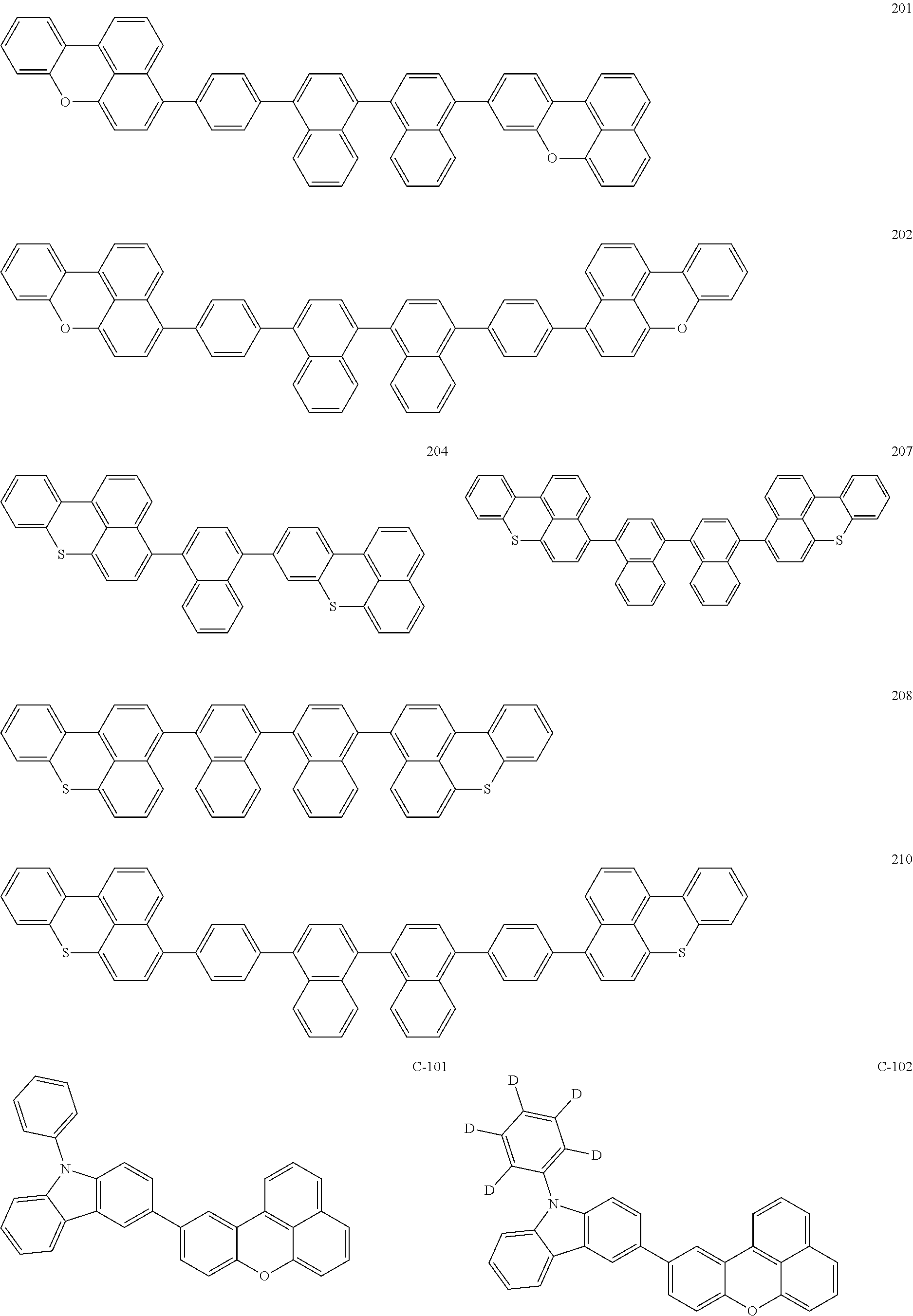

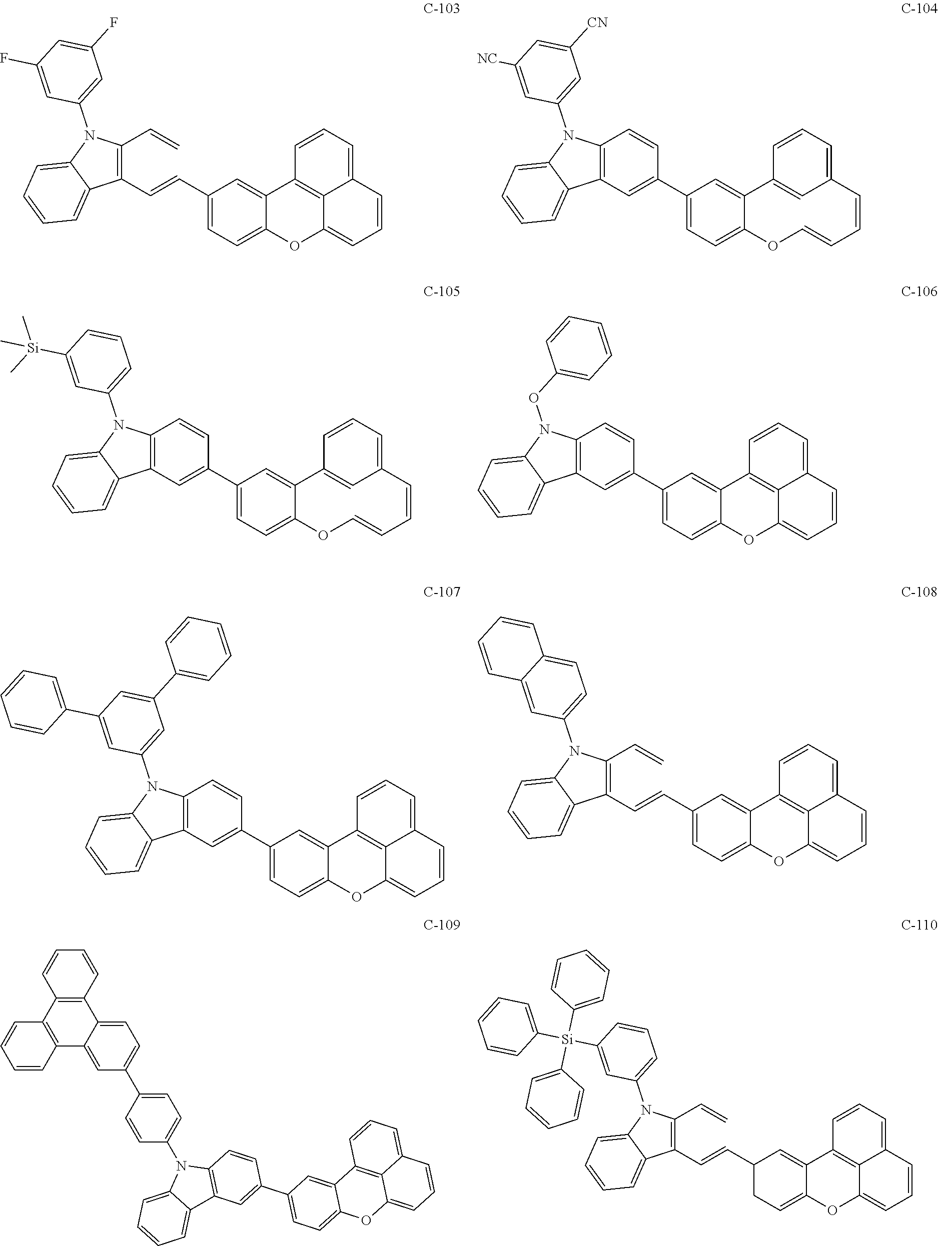

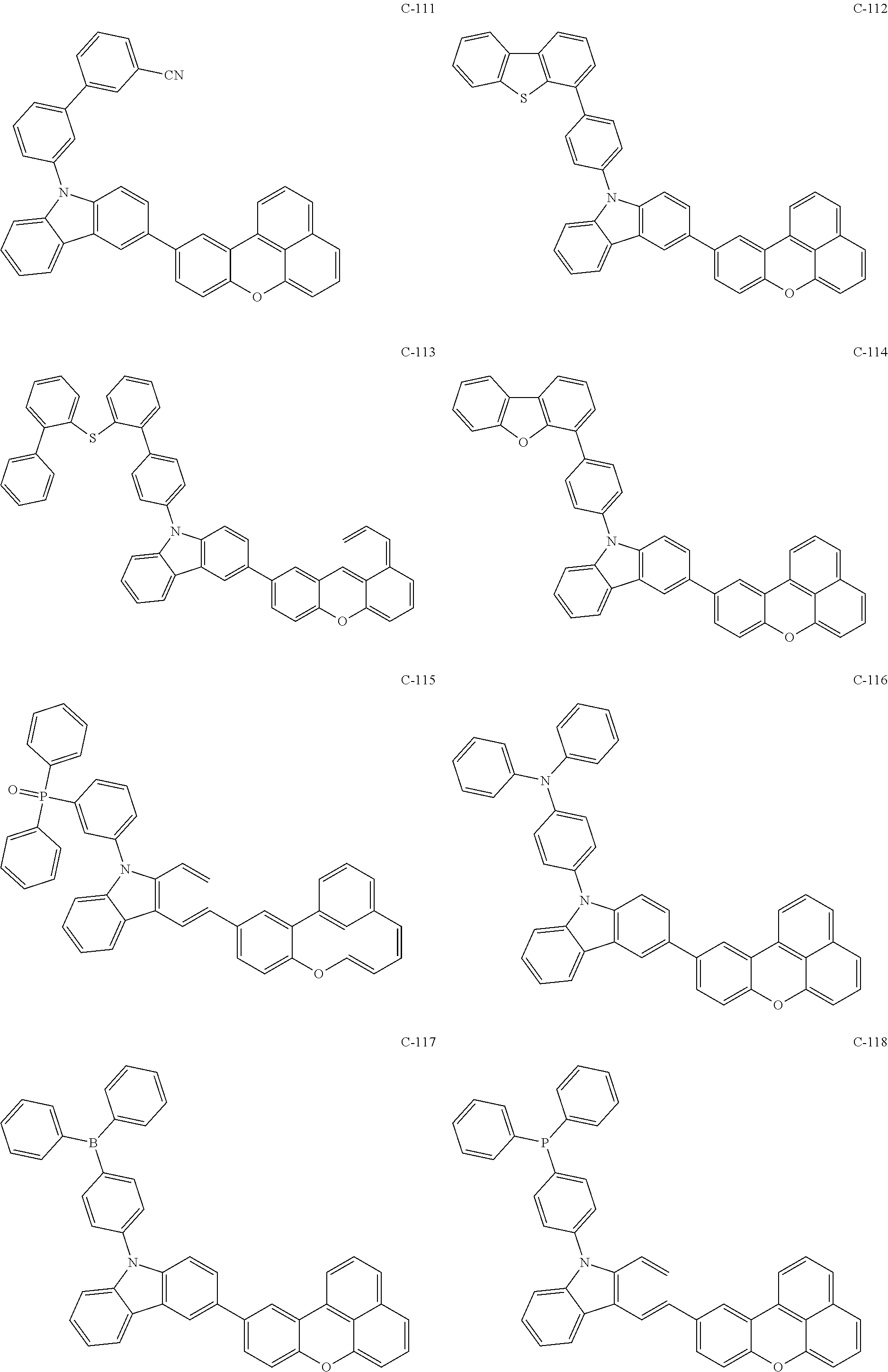

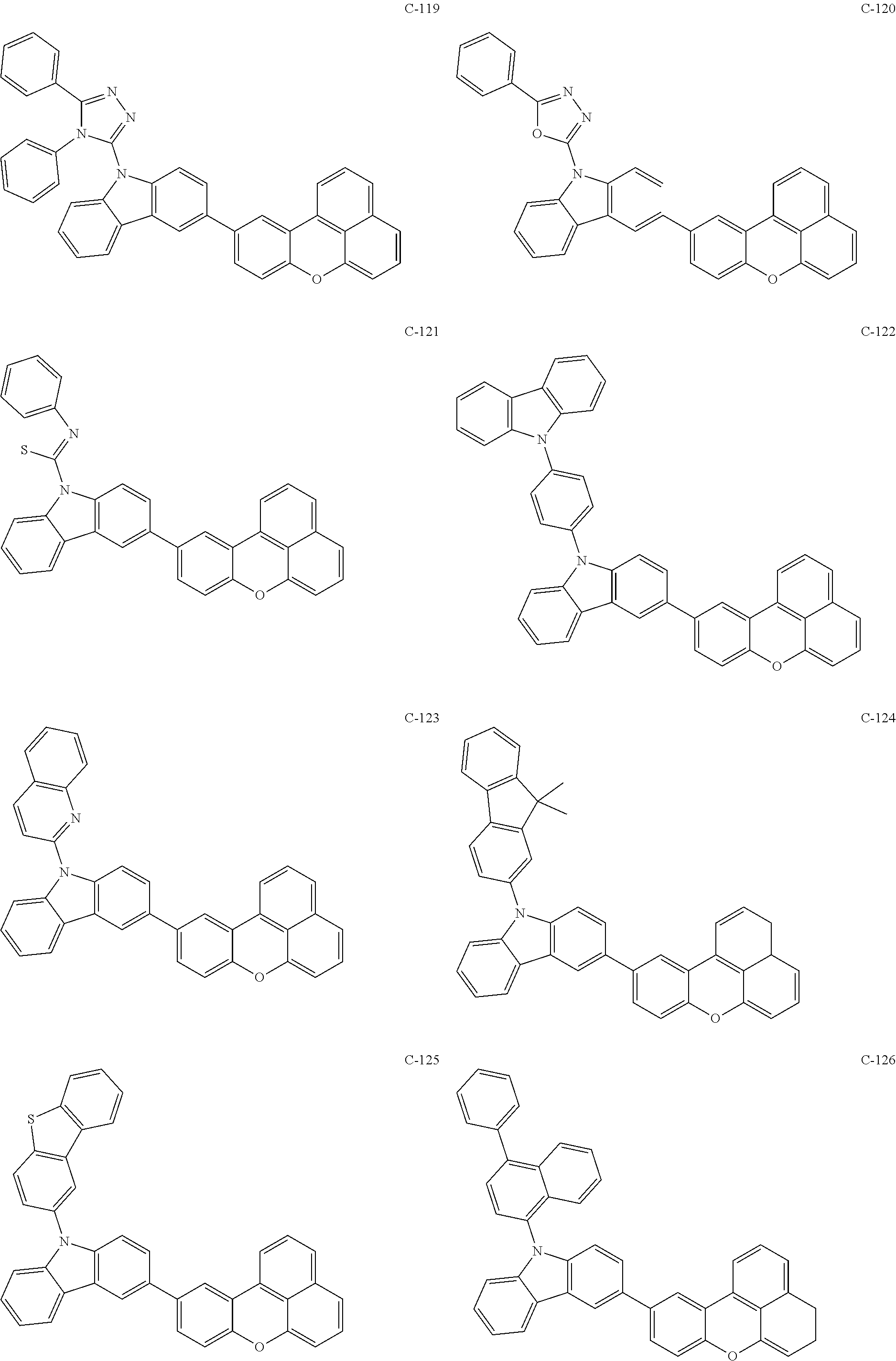

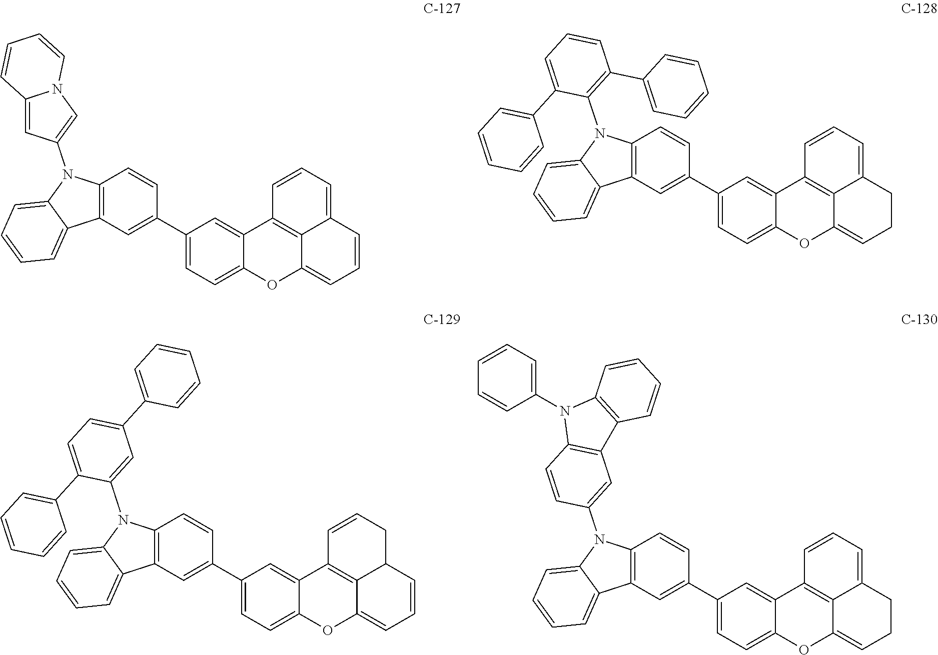 [SMILES: c1cc2c3c(cccc3c1)-c1cc(-c3ccc4c(c3)c3ccccc3n4-c3cc4ccccn4c3)ccc1O2 ; C1=C2Oc3ccc(-c4ccc5c(c4)c4ccccc4n5-c4c(-c5ccccc5)cccc4-c4ccccc4)cc3-c3cccc(c32)CC1 ; C1=CC2CC=CC3=C2C(=C1)Oc1ccc(-c2ccc4c(c2)c2ccccc2n4-c2cc(-c4ccccc4)ccc2-c2ccccc2)cc13 ; C1=C2Oc3ccc(-c4ccc5c(c4)c4ccccc4n5-c4ccc5c(c4)c4ccccc4n5-c4ccccc4)cc3-c3cccc(c32)CC1]

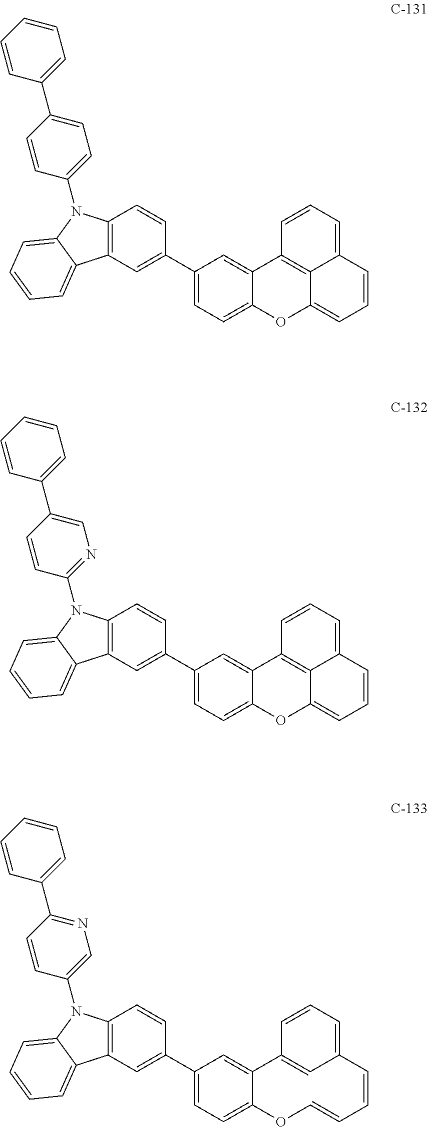 [SMILES: c1ccc(-c2ccc(-n3c4ccccc4c4cc(-c5ccc6c(c5)-c5cccc7cccc(c57)O6)ccc43)cc2)cc1 ; c1ccc(-c2ccc(-n3c4ccccc4c4cc(-c5ccc6c(c5)-c5cccc7cccc(c57)O6)ccc43)nc2)cc1 ; C1=C\c2cccc(c2)-c2cc(-c3ccc4c(c3)c3ccccc3n4-c3ccc(-c4ccccc4)nc3)ccc2O/C=C/1]

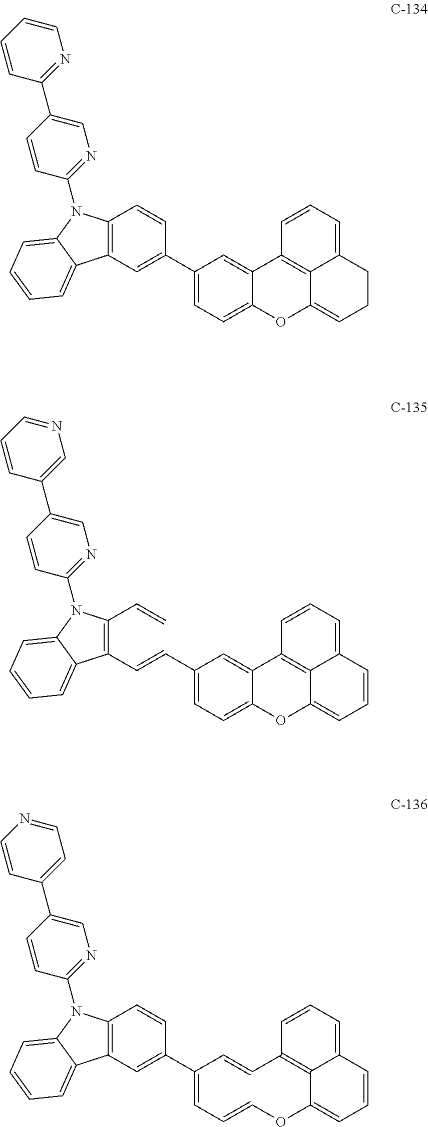 [SMILES: C1=C2Oc3ccc(-c4ccc5c(c4)c4ccccc4n5-c4ccc(-c5ccccn5)cn4)cc3-c3cccc(c32)CC1 ; C=Cc1c(/C=C/c2ccc3c(c2)-c2cccc4cccc(c24)O3)c2ccccc2n1-c1ccc(-c2cccnc2)cn1 ; C1=C\Oc2cccc3cccc(c23)/C=C/C(c2ccc3c(c2)c2ccccc2n3-c2ccc(-c3ccncc3)cn2)=C\1]

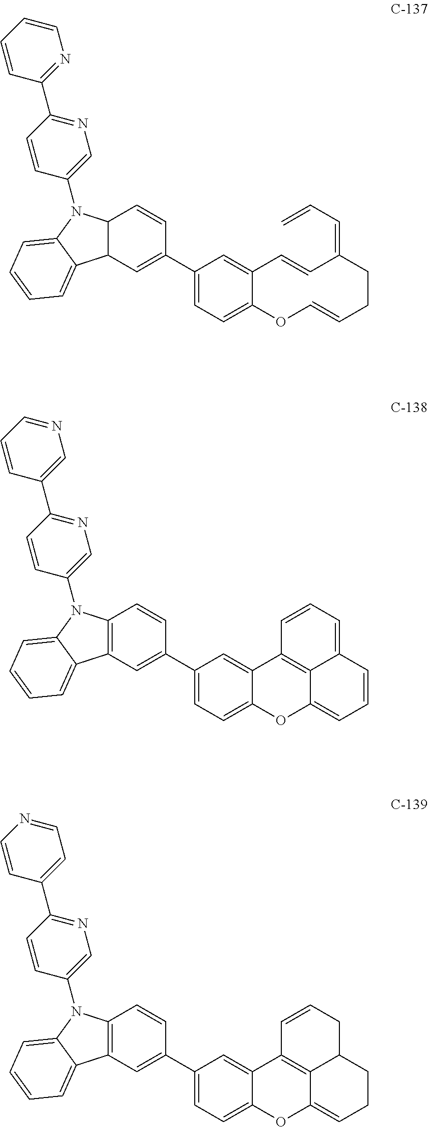 [SMILES: C=C/C=C1\C=C\c2cc(C3=CC4c5ccccc5N(c5ccc(-c6ccccn6)nc5)C4C=C3)ccc2O/C=C/CC1 ; c1cncc(-c2ccc(-n3c4ccccc4c4cc(-c5ccc6c(c5)-c5cccc7cccc(c57)O6)ccc43)cn2)c1 ; C1=CC2=C3C(=CCCC3C1)Oc1ccc(-c3ccc4c(c3)c3ccccc3n4-c3ccc(-c4ccncc4)nc3)cc12]

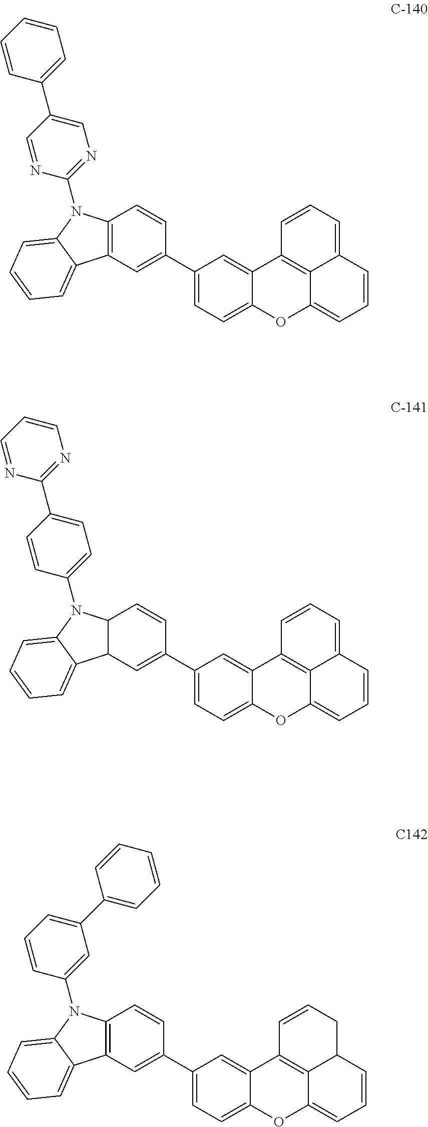 [SMILES: c1ccc(-c2cnc(-n3c4ccccc4c4cc(-c5ccc6c(c5)-c5cccc7cccc(c57)O6)ccc43)nc2)cc1 ; C1=CC2C(C=C1c1ccc3c(c1)-c1cccc4cccc(c14)O3)c1ccccc1N2c1ccc(-c2ncccn2)cc1 ; C1=CC2CC=CC3=C2C(=C1)Oc1ccc(-c2ccc4c(c2)c2ccccc2n4-c2cccc(-c4ccccc4)c2)cc13]

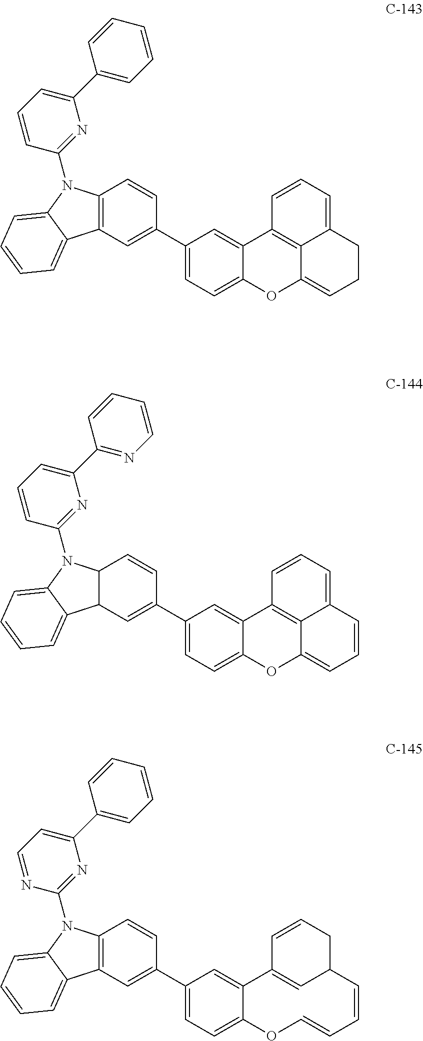 [SMILES: C1=C2Oc3ccc(-c4ccc5c(c4)c4ccccc4n5-c4cccc(-c5ccccc5)n4)cc3-c3cccc(c32)CC1 ; C1=CC2C(C=C1c1ccc3c(c1)-c1cccc4cccc(c14)O3)c1ccccc1N2c1cccc(-c2ccccn2)n1 ; C1=CC2=CC(/C=C\C=C\Oc3ccc(-c4ccc5c(c4)c4ccccc4n5-c4nccc(-c5ccccc5)n4)cc32)C1]

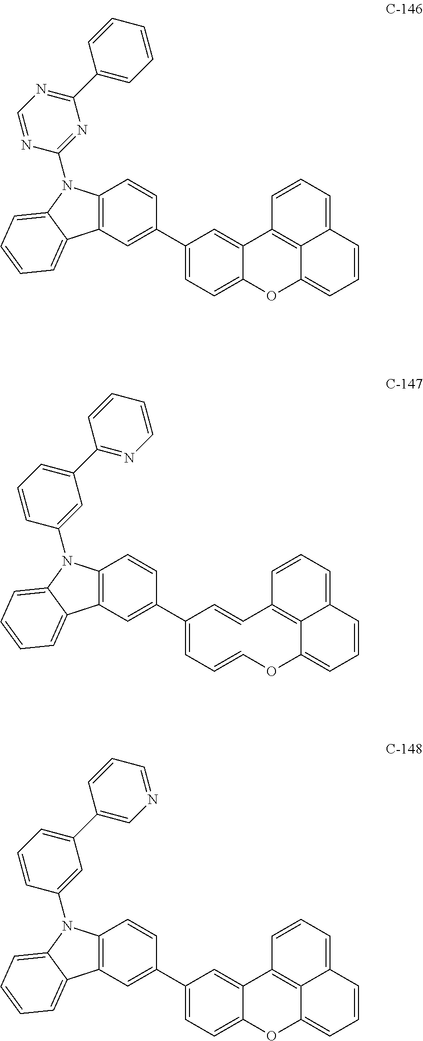 [SMILES: c1ccc(-c2ncnc(-n3c4ccccc4c4cc(-c5ccc6c(c5)-c5cccc7cccc(c57)O6)ccc43)n2)cc1 ; C1=C\Oc2cccc3cccc(c23)/C=C/C(c2ccc3c(c2)c2ccccc2n3-c2cccc(-c3ccccn3)c2)=C\1 ; c1cncc(-c2cccc(-n3c4ccccc4c4cc(-c5ccc6c(c5)-c5cccc7cccc(c57)O6)ccc43)c2)c1]

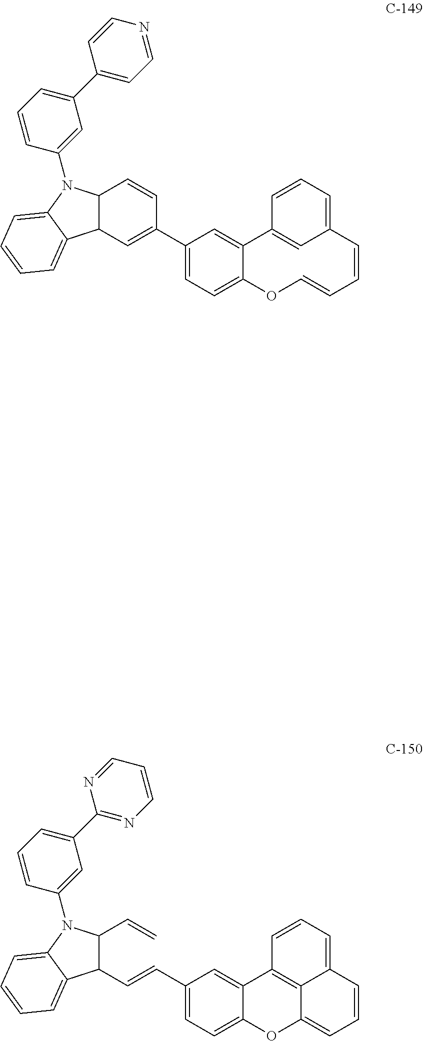 [SMILES: C1=CC2C(C=C1c1ccc3c(c1)-c1cccc(c1)/C=C\C=C\O3)c1ccccc1N2c1cccc(-c2ccncc2)c1 ; C=CC1C(/C=C/c2ccc3c(c2)-c2cccc4cccc(c24)O3)c2ccccc2N1c1cccc(-c2ncccn2)c1]

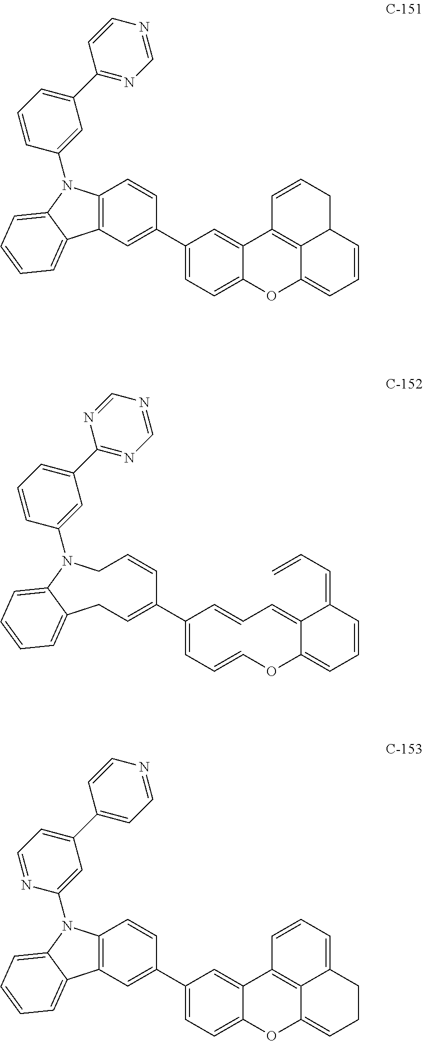 [SMILES: C1=CC2CC=CC3=C2C(=C1)Oc1ccc(-c2ccc4c(c2)c2ccccc2n4-c2cccc(-c4ccncn4)c2)cc13 ; C=C\C=c1\cccc2\c1=C/C=C/C(C1=C/Cc3ccccc3N(c3cccc(-c4ncncn4)c3)C/C=C\1)=C\C=C\O2 ; C1=C2Oc3ccc(-c4ccc5c(c4)c4ccccc4n5-c4cc(-c5ccncc5)ccn4)cc3-c3cccc(c32)CC1]

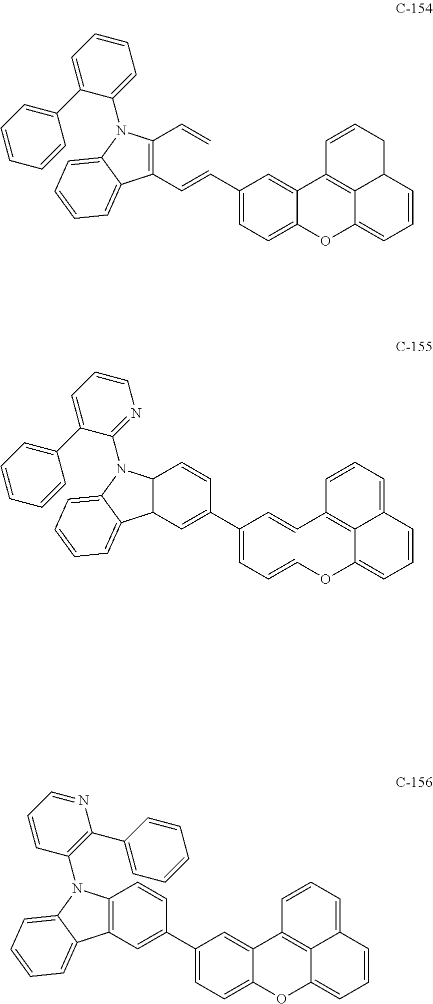 [SMILES: C=Cc1c(/C=C/c2ccc3c(c2)C2=C4C(=CC=CC4CC=C2)O3)c2ccccc2n1-c1ccccc1-c1ccccc1 ; C1=CC2C(C=C1C1=C/C=C/Oc3cccc4cccc(c34)\C=C\1)c1ccccc1N2c1ncccc1-c1ccccc1 ; c1ccc(-c2ncccc2-n2c3ccccc3c3cc(-c4ccc5c(c4)-c4cccc6cccc(c46)O5)ccc32)cc1]

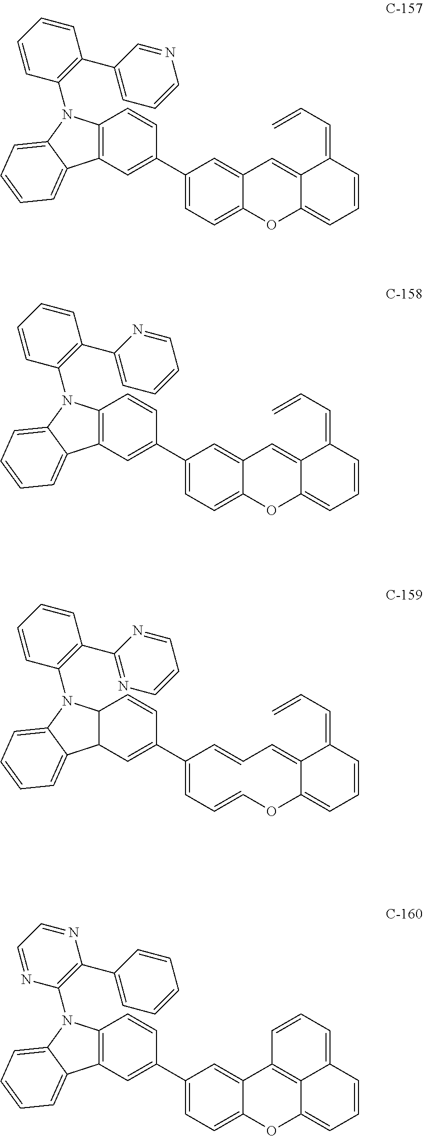 [SMILES: C=C/C=c1/cccc2c1=Cc1cc(-c3ccc4c(c3)c3ccccc3n4-c3ccccc3-c3cccnc3)ccc1O2 ; C=C/C=c1/cccc2c1=Cc1cc(-c3ccc4c(c3)c3ccccc3n4-c3ccccc3-c3ccccn3)ccc1O2 ; C=C/C=c1/cccc2/c1=C\C=C\C(C1=CC3c4ccccc4N(c4ccccc4-c4ncccn4)C3C=C1)=C/C=C/O2 ; c1ccc(-c2nccnc2-n2c3ccccc3c3cc(-c4ccc5c(c4)-c4cccc6cccc(c46)O5)ccc32)cc1]

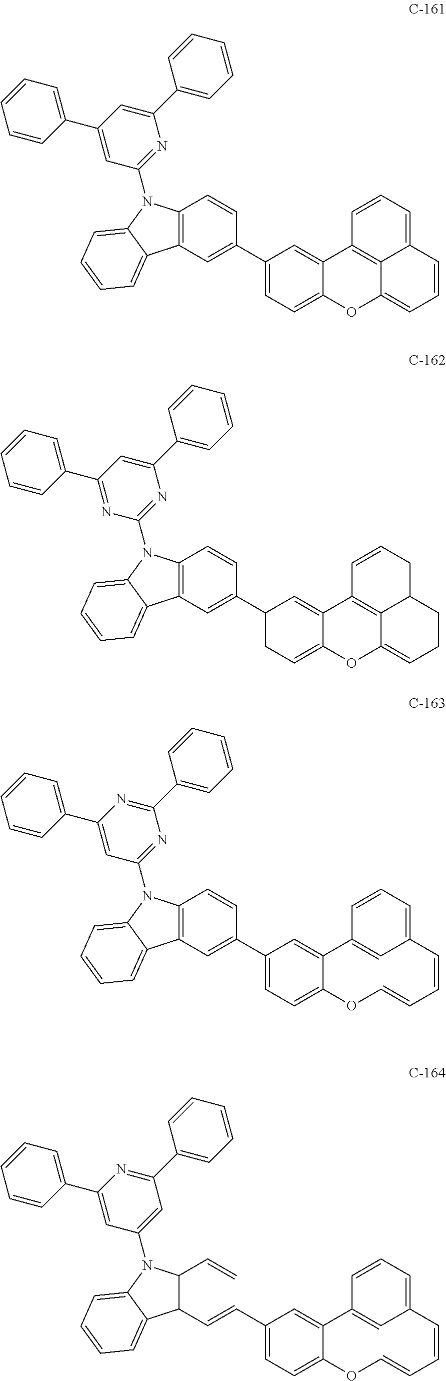 [SMILES: c1ccc(-c2cc(-c3ccccc3)nc(-n3c4ccccc4c4cc(-c5ccc6c(c5)-c5cccc7cccc(c57)O6)ccc43)c2)cc1 ; C1=CC2=C3C(=CCCC3C1)OC1=CCC(c3ccc4c(c3)c3ccccc3n4-c3nc(-c4ccccc4)cc(-c4ccccc4)n3)C=C12 ; C1=C\c2cccc(c2)-c2cc(-c3ccc4c(c3)c3ccccc3n4-c3cc(-c4ccccc4)nc(-c4ccccc4)n3)ccc2O/C=C/1 ; C=CC1C(/C=C/c2ccc3c(c2)-c2cccc(c2)/C=C\C=C\O3)c2ccccc2N1c1cc(-c2ccccc2)nc(-c2ccccc2)c1]

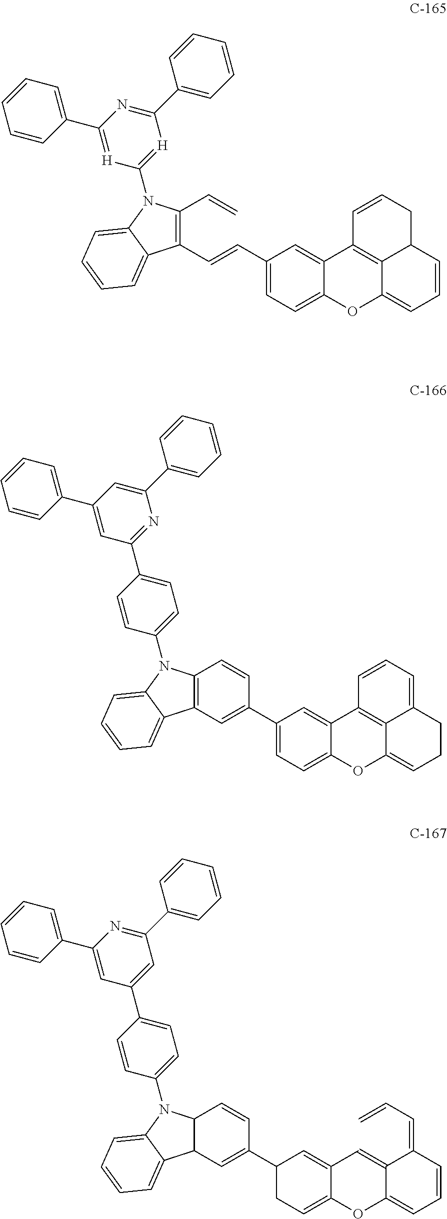

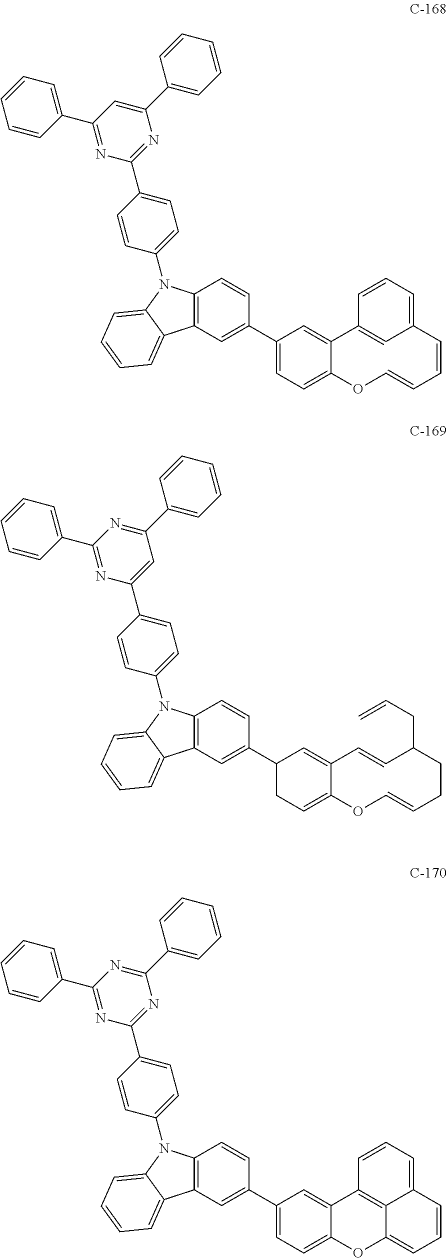 [SMILES: C1=C\c2cccc(c2)-c2cc(-c3ccc4c(c3)c3ccccc3n4-c3ccc(-c4nc(-c5ccccc5)cc(-c5ccccc5)n4)cc3)ccc2O/C=C/1 ; C=CCC1/C=C/C2=CC(c3ccc4c(c3)c3ccccc3n4-c3ccc(-c4cc(-c5ccccc5)nc(-c5ccccc5)n4)cc3)CC=C2O/C=C/CC1 ; c1ccc(-c2nc(-c3ccccc3)nc(-c3ccc(-n4c5ccccc5c5cc(-c6ccc7c(c6)-c6cccc8cccc(c68)O7)ccc54)cc3)n2)cc1]

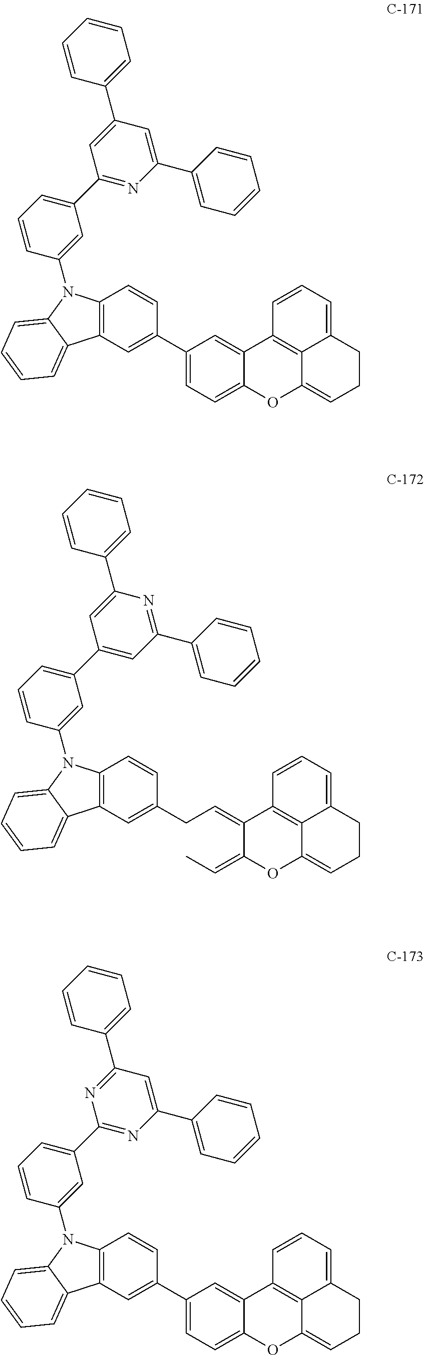 [SMILES: C1=C2Oc3ccc(-c4ccc5c(c4)c4ccccc4n5-c4cccc(-c5cc(-c6ccccc6)cc(-c6ccccc6)n5)c4)cc3-c3cccc(c32)CC1 ; C/C=C1/OC2=CCCc3cccc(c32)/C1=C/Cc1ccc2c(c1)c1ccccc1n2-c1cccc(-c2cc(-c3ccccc3)nc(-c3ccccc3)c2)c1 ; C1=C2Oc3ccc(-c4ccc5c(c4)c4ccccc4n5-c4cccc(-c5nc(-c6ccccc6)cc(-c6ccccc6)n5)c4)cc3-c3cccc(c32)CC1]

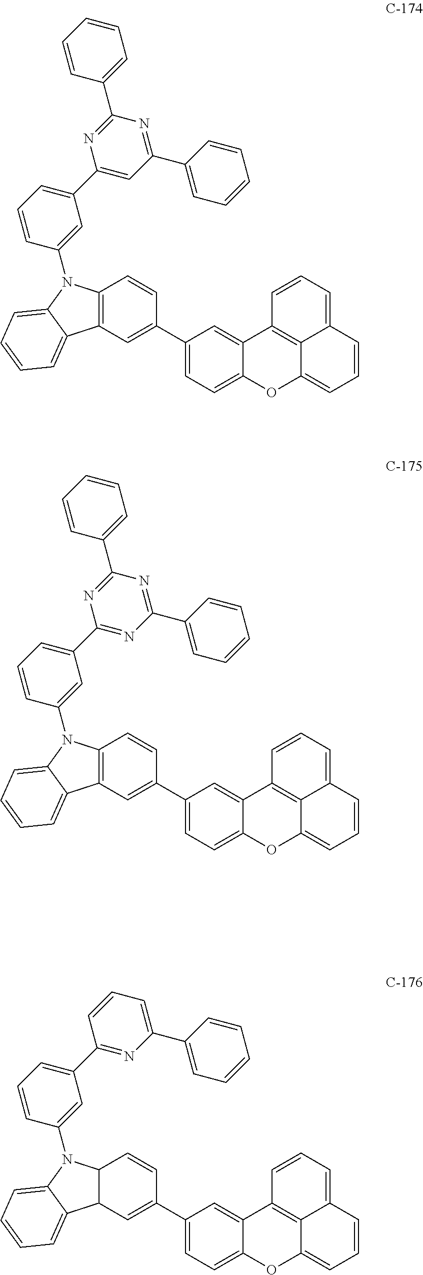 [SMILES: c1ccc(-c2cc(-c3cccc(-n4c5ccccc5c5cc(-c6ccc7c(c6)-c6cccc8cccc(c68)O7)ccc54)c3)nc(-c3ccccc3)n2)cc1 ; c1ccc(-c2nc(-c3ccccc3)nc(-c3cccc(-n4c5ccccc5c5cc(-c6ccc7c(c6)-c6cccc8cccc(c68)O7)ccc54)c3)n2)cc1 ; C1=CC2C(C=C1c1ccc3c(c1)-c1cccc4cccc(c14)O3)c1ccccc1N2c1cccc(-c2cccc(-c3ccccc3)n2)c1]

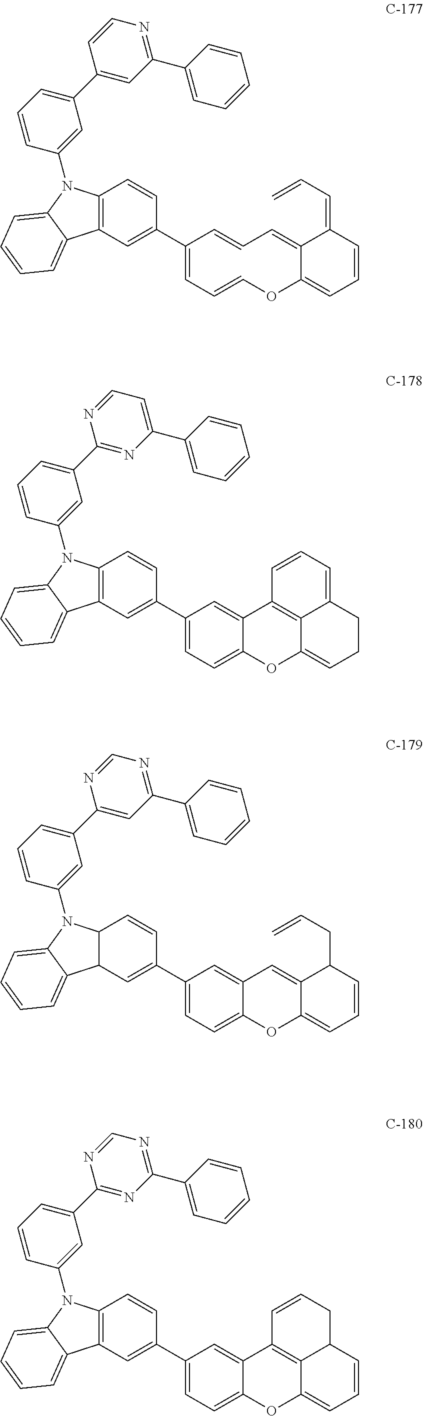 [SMILES: C=C/C=c1/cccc2/c1=C\C=C\C(c1ccc3c(c1)c1ccccc1n3-c1cccc(-c3ccnc(-c4ccccc4)c3)c1)=C/C=C/O2 ; C1=C2Oc3ccc(-c4ccc5c(c4)c4ccccc4n5-c4cccc(-c5nccc(-c6ccccc6)n5)c4)cc3-c3cccc(c32)CC1 ; C=CCC1C=CC=C2Oc3ccc(C4=CC5c6ccccc6N(c6cccc(-c7cc(-c8ccccc8)ncn7)c6)C5C=C4)cc3C=C21 ; C1=CC2CC=CC3=C2C(=C1)Oc1ccc(-c2ccc4c(c2)c2ccccc2n4-c2cccc(-c4ncnc(-c5ccccc5)n4)c2)cc13]

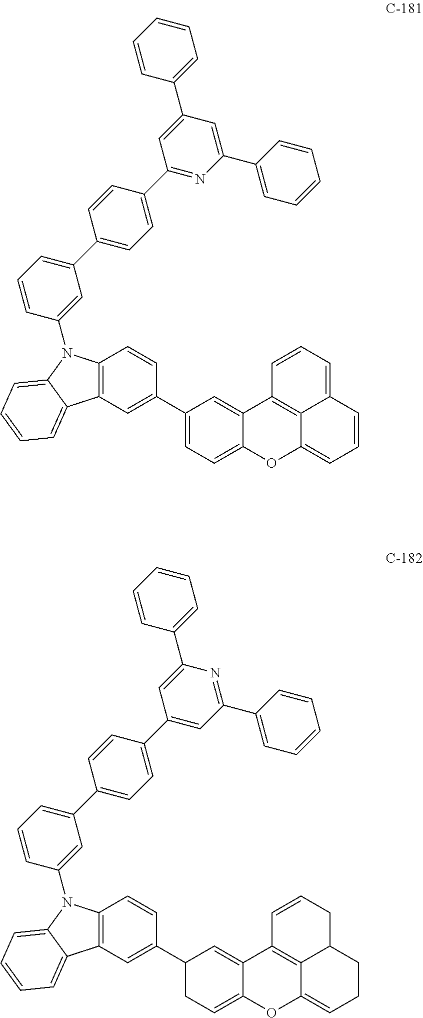 [SMILES: c1ccc(-c2cc(-c3ccccc3)nc(-c3ccc(-c4cccc(-n5c6ccccc6c6cc(-c7ccc8c(c7)-c7cccc9cccc(c79)O8)ccc65)c4)cc3)c2)cc1 ; C1=CC2=C3C(=CCCC3C1)OC1=CCC(c3ccc4c(c3)c3ccccc3n4-c3cccc(-c4ccc(-c5cc(-c6ccccc6)nc(-c6ccccc6)c5)cc4)c3)C=C12]

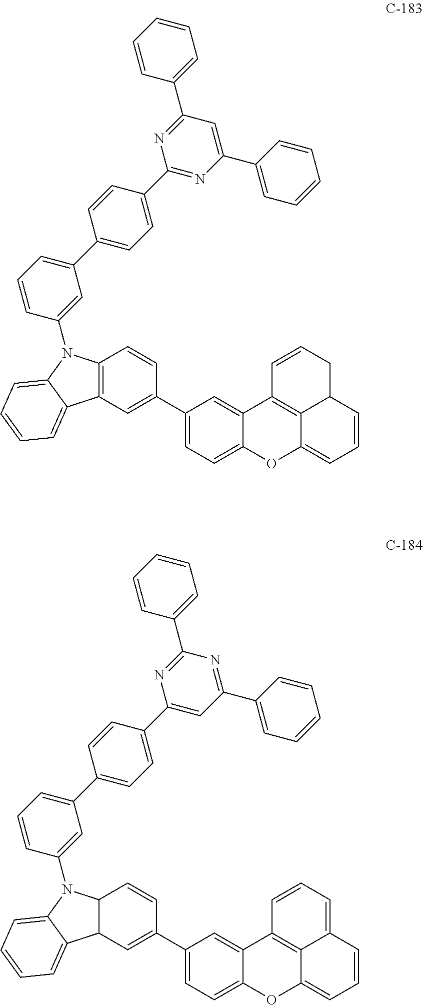 [SMILES: C1=CC2CC=CC3=C2C(=C1)Oc1ccc(-c2ccc4c(c2)c2ccccc2n4-c2cccc(-c4ccc(-c5nc(-c6ccccc6)cc(-c6ccccc6)n5)cc4)c2)cc13 ; C1=CC2C(C=C1c1ccc3c(c1)-c1cccc4cccc(c14)O3)c1ccccc1N2c1cccc(-c2ccc(-c3cc(-c4ccccc4)nc(-c4ccccc4)n3)cc2)c1]

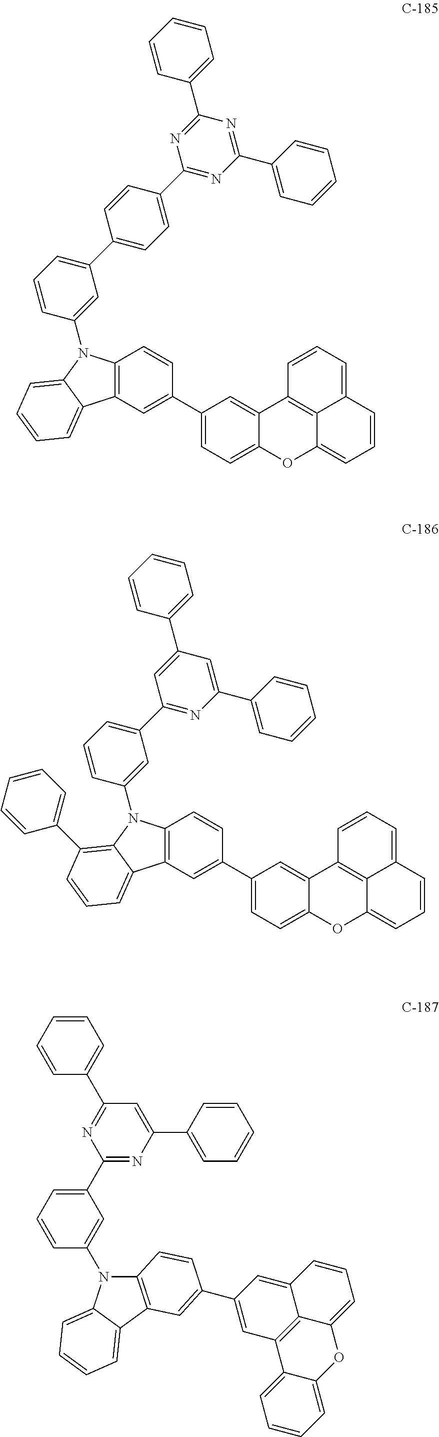 [SMILES: c1ccc(-c2nc(-c3ccccc3)nc(-c3ccc(-c4cccc(-n5c6ccccc6c6cc(-c7ccc8c(c7)-c7cccc9cccc(c79)O8)ccc65)c4)cc3)n2)cc1 ; c1ccc(-c2cc(-c3ccccc3)nc(-c3cccc(-n4c5ccc(-c6ccc7c(c6)-c6cccc8cccc(c68)O7)cc5c5cccc(-c6ccccc6)c54)c3)c2)cc1 ; c1ccc(-c2cc(-c3ccccc3)nc(-c3cccc(-n4c5ccccc5c5cc(-c6cc7c8c(cccc8c6)Oc6ccccc6-7)ccc54)c3)n2)cc1]

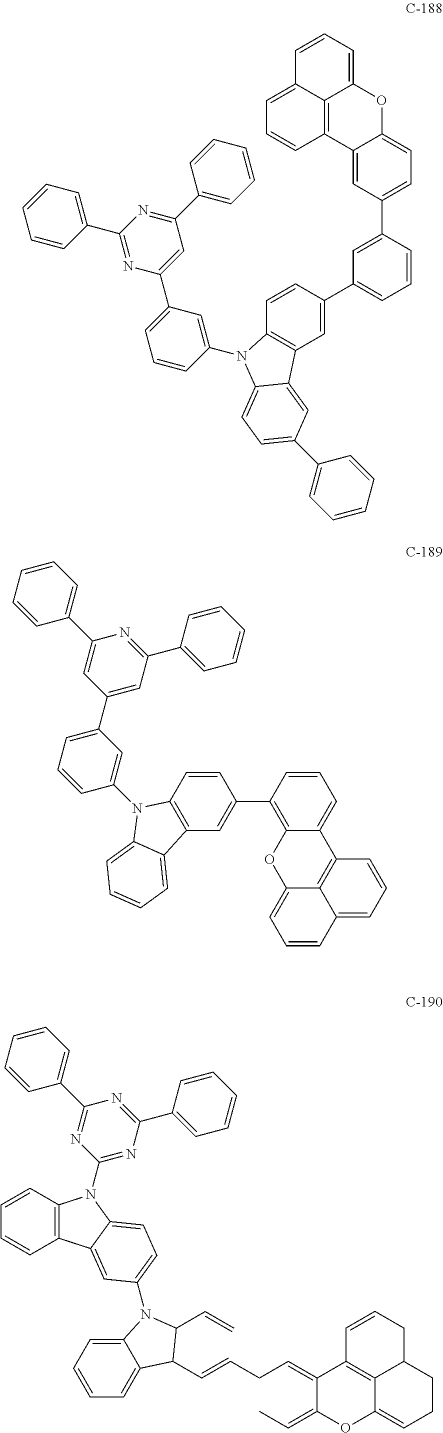 [SMILES: c1ccc(-c2ccc3c(c2)c2cc(-c4cccc(-c5ccc6c(c5)-c5cccc7cccc(c57)O6)c4)ccc2n3-c2cccc(-c3cc(-c4ccccc4)nc(-c4ccccc4)n3)c2)cc1 ; c1ccc(-c2cc(-c3cccc(-n4c5ccccc5c5cc(-c6cccc7c6Oc6cccc8cccc-7c68)ccc54)c3)cc(-c3ccccc3)n2)cc1 ; C=CC1C(/C=C/C/C=C2/C3=C4C(=CCCC4CC=C3)O/C2=C/C)c2ccccc2N1c1ccc2c(c1)c1ccccc1n2-c1nc(-c2ccccc2)nc(-c2ccccc2)n1]

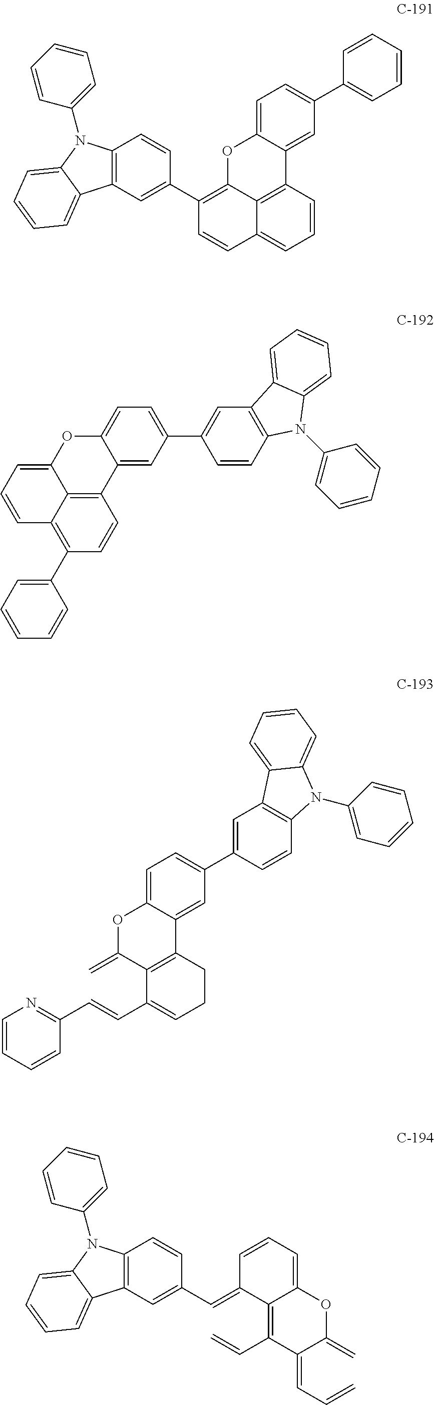 [SMILES: c1ccc(-c2ccc3c(c2)-c2cccc4ccc(-c5ccc6c(c5)c5ccccc5n6-c5ccccc5)c(c24)O3)cc1 ; c1ccc(-c2ccc3c4c(cccc24)Oc2ccc(-c4ccc5c(c4)c4ccccc4n5-c4ccccc4)cc2-3)cc1 ; C=C1Oc2ccc(-c3ccc4c(c3)c3ccccc3n4-c3ccccc3)cc2C2=C1C(/C=C/c1ccccn1)=CCC2 ; C=C/C=C1\C(=C)Oc2ccc/c(=C\c3ccc4c(c3)c3ccccc3n4-c3ccccc3)c2=C1C=C]

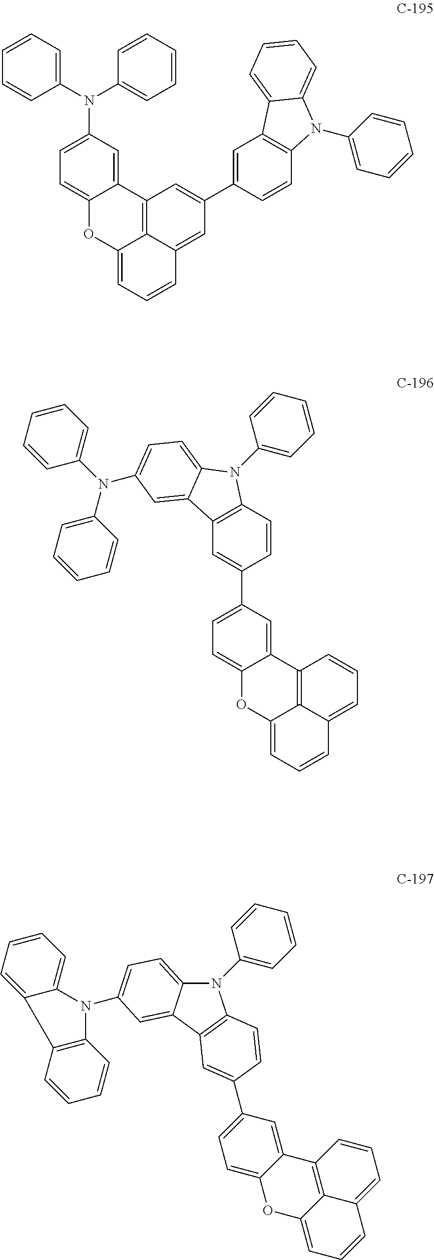 [SMILES: c1ccc(N(c2ccccc2)c2ccc3c(c2)-c2cc(-c4ccc5c(c4)c4ccccc4n5-c4ccccc4)cc4cccc(c24)O3)cc1 ; c1ccc(N(c2ccccc2)c2ccc3c(c2)c2cc(-c4ccc5c(c4)-c4cccc6cccc(c46)O5)ccc2n3-c2ccccc2)cc1 ; c1ccc(-n2c3ccc(-c4ccc5c(c4)-c4cccc6cccc(c46)O5)cc3c3cc(-n4c5ccccc5c5ccccc54)ccc32)cc1]

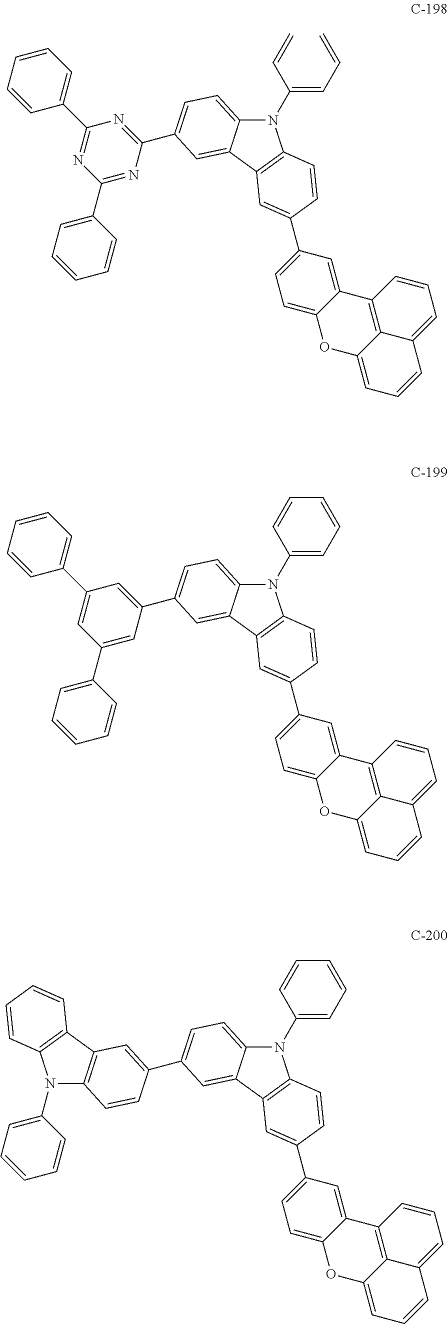 [SMILES: C=C/C=C(\C=C)n1c2ccc(-c3ccc4c(c3)-c3cccc5cccc(c35)O4)cc2c2cc(-c3nc(-c4ccccc4)nc(-c4ccccc4)n3)ccc21 ; c1ccc(-c2cc(-c3ccccc3)cc(-c3ccc4c(c3)c3cc(-c5ccc6c(c5)-c5cccc7cccc(c57)O6)ccc3n4-c3ccccc3)c2)cc1 ; c1ccc(-n2c3ccccc3c3cc(-c4ccc5c(c4)c4cc(-c6ccc7c(c6)-c6cccc8cccc(c68)O7)ccc4n5-c4ccccc4)ccc32)cc1]

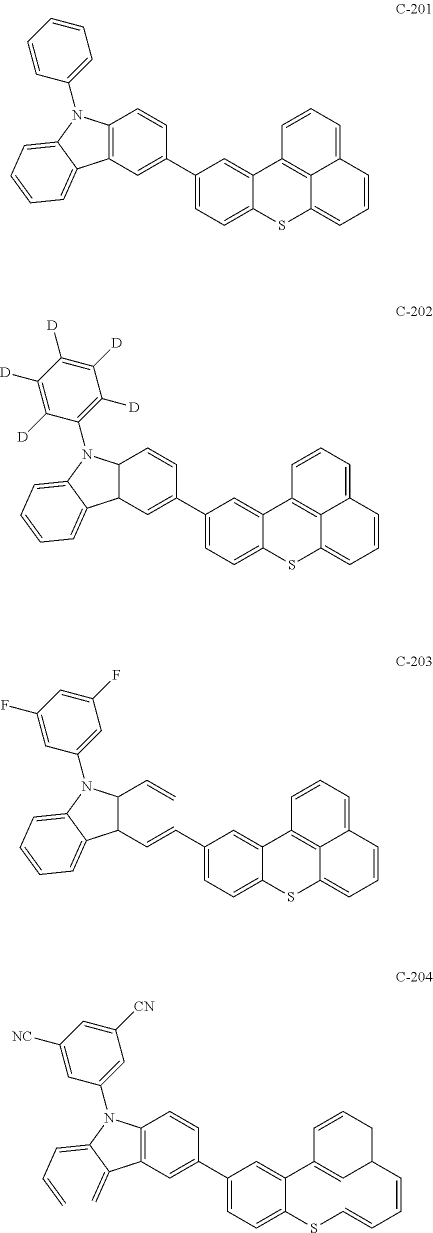 [SMILES: c1ccc(-n2c3ccccc3c3cc(-c4ccc5c(c4)-c4cccc6cccc(c46)S5)ccc32)cc1 ; [2H]c1c([2H])c([2H])c(N2c3ccccc3C3C=C(c4ccc5c(c4)-c4cccc6cccc(c46)S5)C=CC32)c([2H])c1[2H] ; C=CC1C(/C=C/c2ccc3c(c2)-c2cccc4cccc(c24)S3)c2ccccc2N1c1cc(F)cc(F)c1 ; C=C/C=c1\c(=C)c2cc(-c3ccc4c(c3)C3=CC(/C=C\C=C\S4)CC=C3)ccc2n1-c1cc(C#N)cc(C#N)c1]

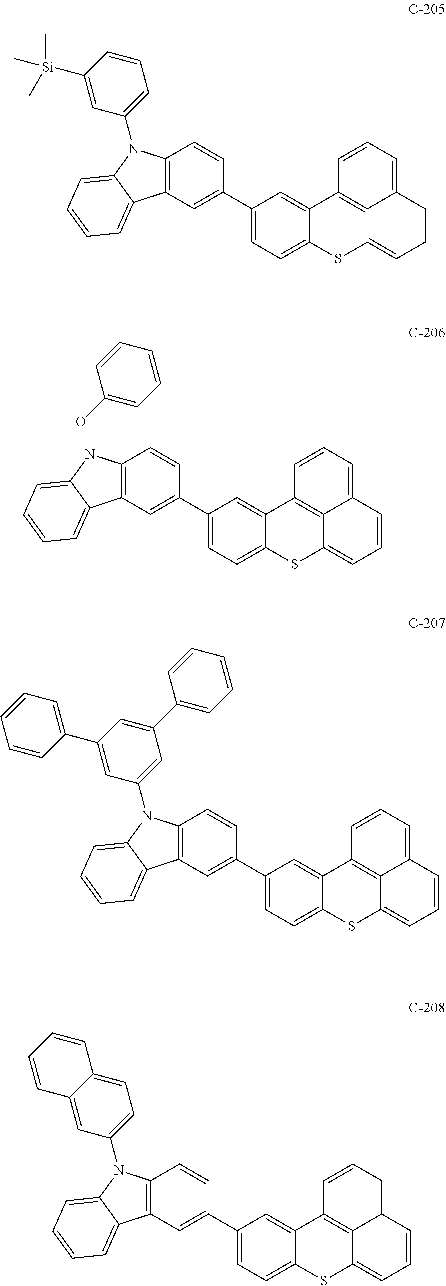

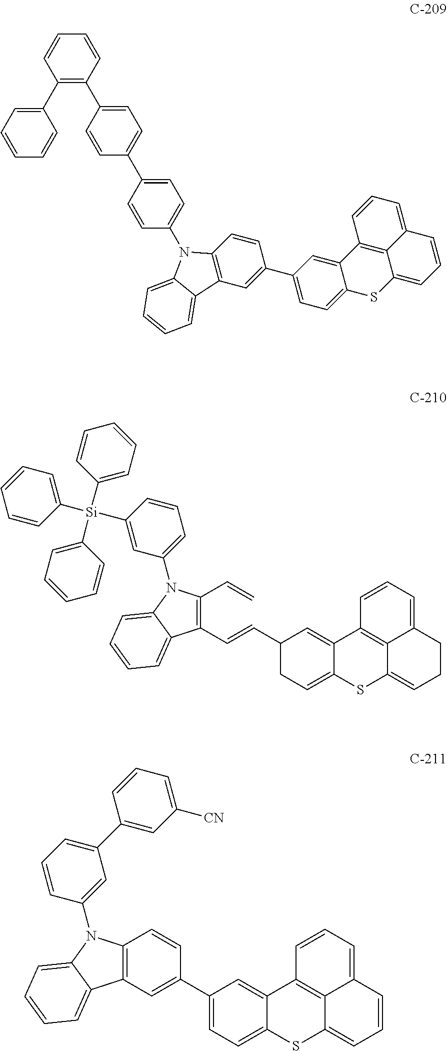 [SMILES: c1ccc(-c2ccccc2-c2ccc(-c3ccc(-n4c5ccccc5c5cc(-c6ccc7c(c6)-c6cccc8cccc(c68)S7)ccc54)cc3)cc2)cc1 ; C=Cc1c(/C=C/C2C=C3C(=CC2)SC2=CCCc4cccc3c42)c2ccccc2n1-c1cccc([Si](c2ccccc2)(c2ccccc2)c2ccccc2)c1 ; N#Cc1cccc(-c2cccc(-n3c4ccccc4c4cc(-c5ccc6c(c5)-c5cccc7cccc(c57)S6)ccc43)c2)c1]

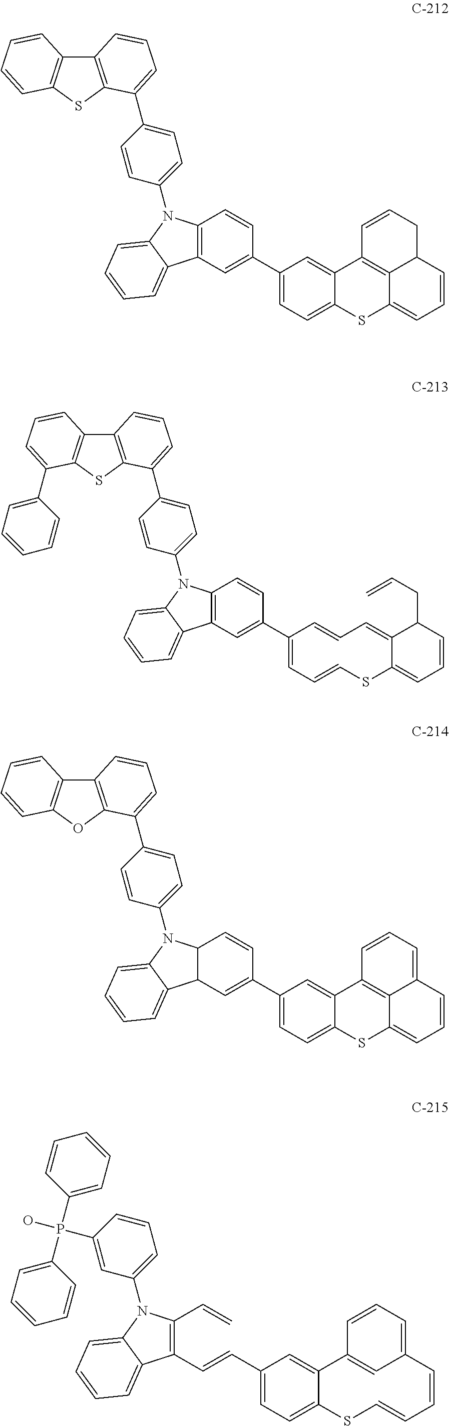

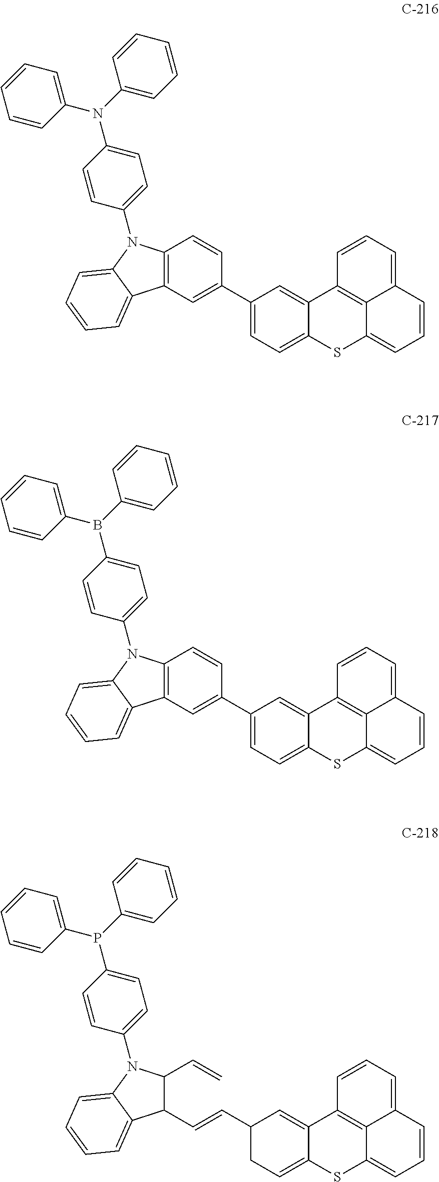 [SMILES: c1ccc(N(c2ccccc2)c2ccc(-n3c4ccccc4c4cc(-c5ccc6c(c5)-c5cccc7cccc(c57)S6)ccc43)cc2)cc1 ; c1ccc(B(c2ccccc2)c2ccc(-n3c4ccccc4c4cc(-c5ccc6c(c5)-c5cccc7cccc(c57)S6)ccc43)cc2)cc1 ; C=CC1C(/C=C/C2C=C3C(=CC2)Sc2cccc4cccc3c24)c2ccccc2N1c1ccc(P(c2ccccc2)c2ccccc2)cc1]

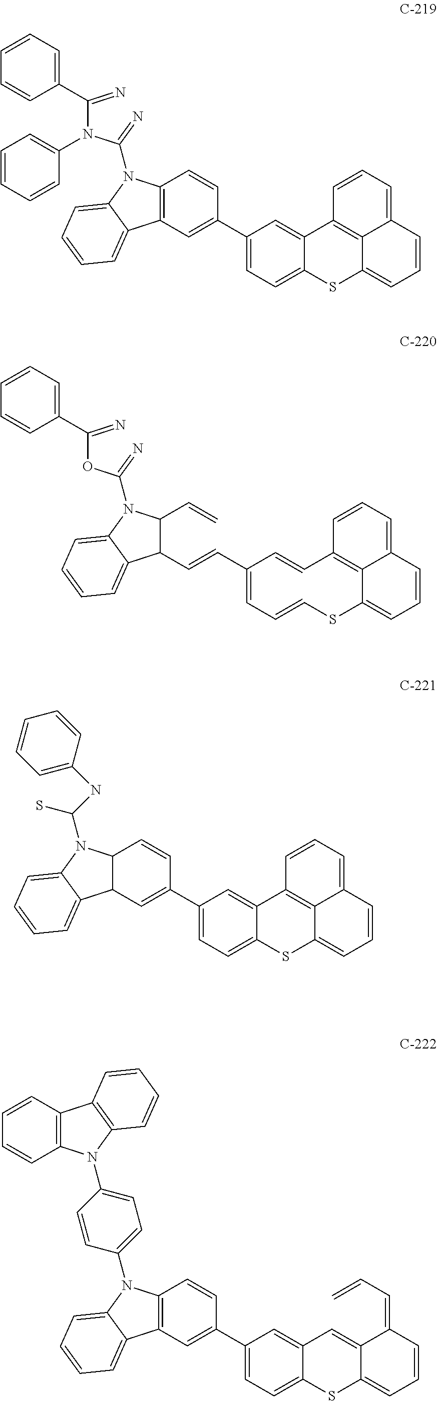 [SMILES: N=C(c1ccccc1)N(C(=N)n1c2ccccc2c2cc(-c3ccc4c(c3)-c3cccc5cccc(c35)S4)ccc21)c1ccccc1 ; C=CC1C(/C=C/C2=C/C=C/Sc3cccc4cccc(c34)\C=C\2)c2ccccc2N1C(=N)OC(=N)c1ccccc1 ; SC(Nc1ccccc1)N1c2ccccc2C2C=C(c3ccc4c(c3)-c3cccc5cccc(c35)S4)C=CC21 ; C=C/C=c1/cccc2c1=Cc1cc(-c3ccc4c(c3)c3ccccc3n4-c3ccc(-n4c5ccccc5c5ccccc54)cc3)ccc1S2]

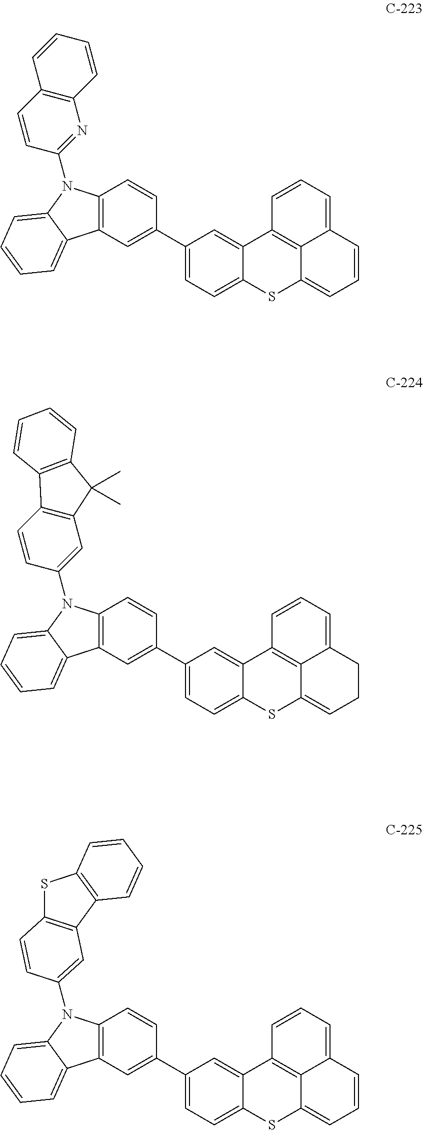 [SMILES: c1ccc2nc(-n3c4ccccc4c4cc(-c5ccc6c(c5)-c5cccc7cccc(c57)S6)ccc43)ccc2c1 ; CC1(C)c2ccccc2-c2ccc(-n3c4ccccc4c4cc(-c5ccc6c(c5)-c5cccc7c5C(=CCC7)S6)ccc43)cc21 ; c1cc2c3c(cccc3c1)-c1cc(-c3ccc4c(c3)c3ccccc3n4-c3ccc4sc5ccccc5c4c3)ccc1S2]

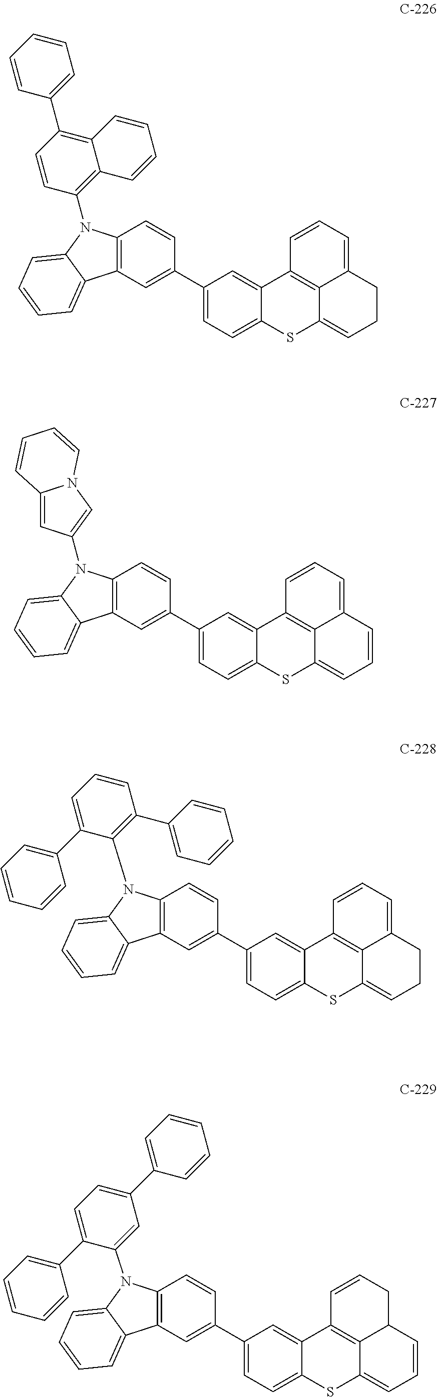 [SMILES: C1=C2Sc3ccc(-c4ccc5c(c4)c4ccccc4n5-c4ccc(-c5ccccc5)c5ccccc45)cc3-c3cccc(c32)CC1 ; c1cc2c3c(cccc3c1)-c1cc(-c3ccc4c(c3)c3ccccc3n4-c3cc4ccccn4c3)ccc1S2 ; C1=C2Sc3ccc(-c4ccc5c(c4)c4ccccc4n5-c4c(-c5ccccc5)cccc4-c4ccccc4)cc3-c3cccc(c32)CC1 ; C1=CC2CC=CC3=C2C(=C1)Sc1ccc(-c2ccc4c(c2)c2ccccc2n4-c2cc(-c4ccccc4)ccc2-c2ccccc2)cc13]

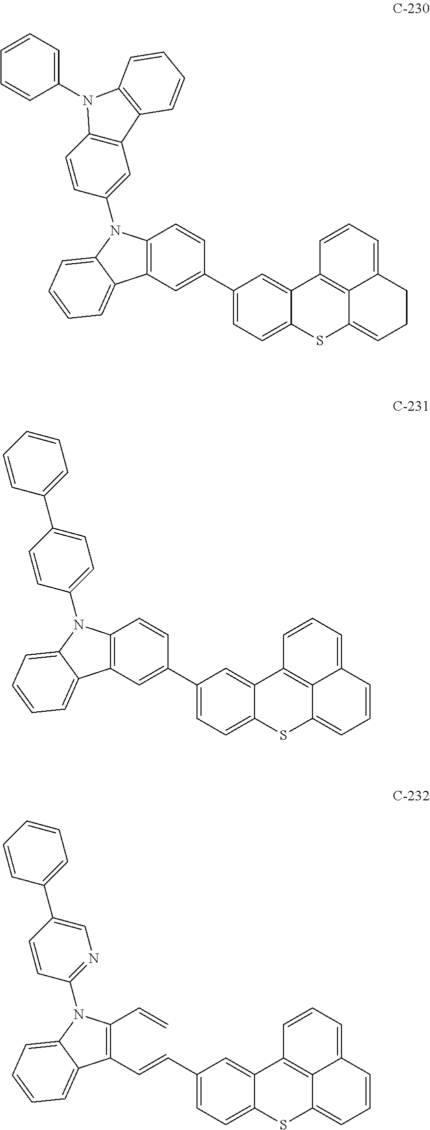 [SMILES: C1=C2Sc3ccc(-c4ccc5c(c4)c4ccccc4n5-c4ccc5c(c4)c4ccccc4n5-c4ccccc4)cc3-c3cccc(c32)CC1 ; c1ccc(-c2ccc(-n3c4ccccc4c4cc(-c5ccc6c(c5)-c5cccc7cccc(c57)S6)ccc43)cc2)cc1 ; C=Cc1c(/C=C/c2ccc3c(c2)-c2cccc4cccc(c24)S3)c2ccccc2n1-c1ccc(-c2ccccc2)cn1]

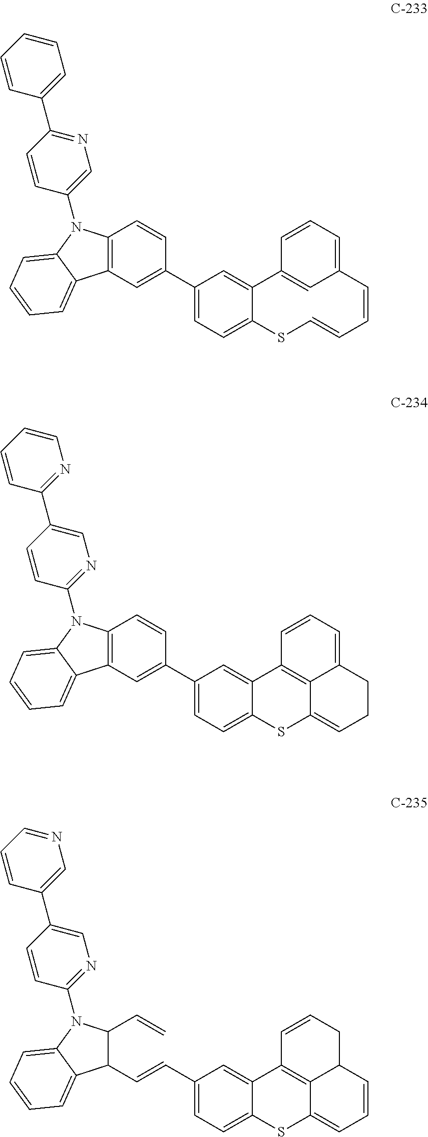 [SMILES: C1=C\c2cccc(c2)-c2cc(-c3ccc4c(c3)c3ccccc3n4-c3ccc(-c4ccccc4)nc3)ccc2S/C=C/1 ; C1=C2Sc3ccc(-c4ccc5c(c4)c4ccccc4n5-c4ccc(-c5ccccn5)cn4)cc3-c3cccc(c32)CC1 ; C=CC1C(/C=C/c2ccc3c(c2)C2=C4C(=CC=CC4CC=C2)S3)c2ccccc2N1c1ccc(-c2cccnc2)cn1]

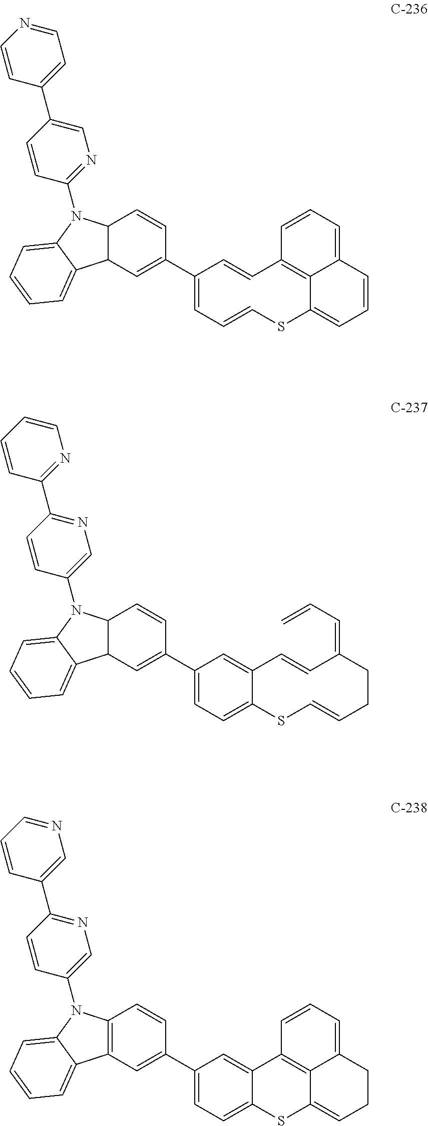 [SMILES: C1=CC2C(C=C1C1=C/C=C/Sc3cccc4cccc(c34)\C=C\1)c1ccccc1N2c1ccc(-c2ccncc2)cn1 ; C=C/C=C1\C=C\c2cc(C3=CC4c5ccccc5N(c5ccc(-c6ccccn6)nc5)C4C=C3)ccc2S/C=C/CC1 ; C1=C2Sc3ccc(-c4ccc5c(c4)c4ccccc4n5-c4ccc(-c5cccnc5)nc4)cc3-c3cccc(c32)CC1]

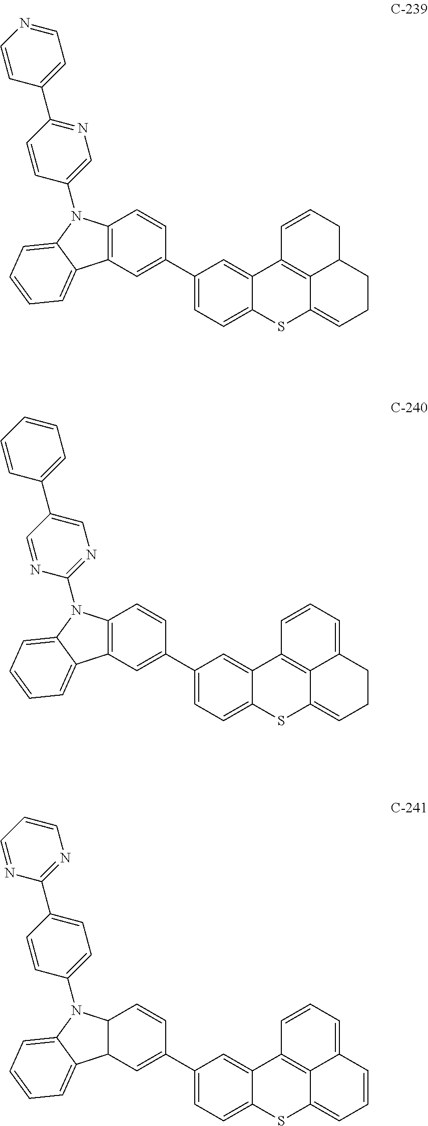 [SMILES: C1=CC2=C3C(=CCCC3C1)Sc1ccc(-c3ccc4c(c3)c3ccccc3n4-c3ccc(-c4ccncc4)nc3)cc12 ; C1=C2Sc3ccc(-c4ccc5c(c4)c4ccccc4n5-c4ncc(-c5ccccc5)cn4)cc3-c3cccc(c32)CC1 ; C1=CC2C(C=C1c1ccc3c(c1)-c1cccc4cccc(c14)S3)c1ccccc1N2c1ccc(-c2ncccn2)cc1]

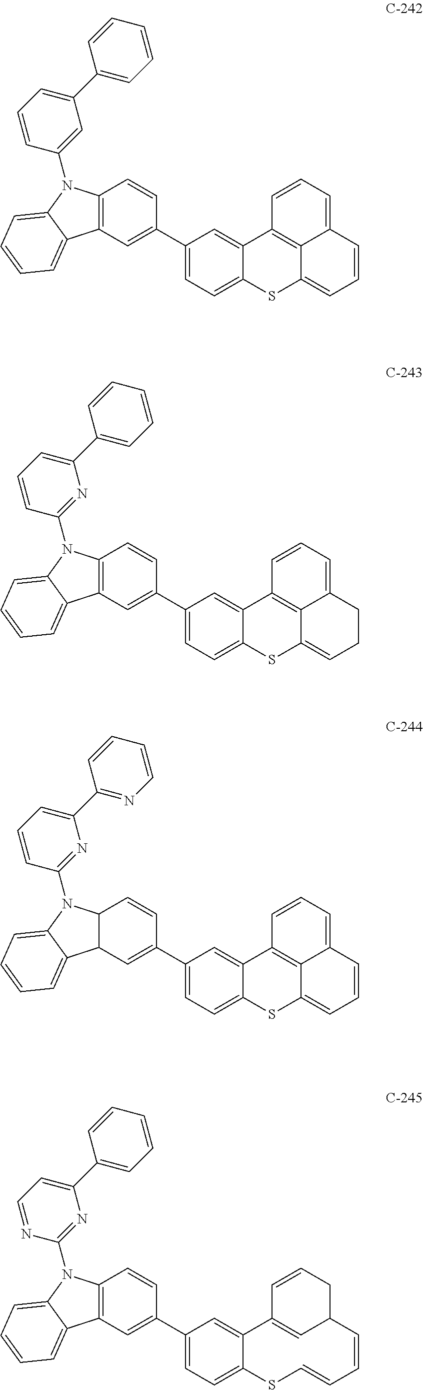 [SMILES: c1ccc(-c2cccc(-n3c4ccccc4c4cc(-c5ccc6c(c5)-c5cccc7cccc(c57)S6)ccc43)c2)cc1 ; C1=C2Sc3ccc(-c4ccc5c(c4)c4ccccc4n5-c4cccc(-c5ccccc5)n4)cc3-c3cccc(c32)CC1 ; C1=CC2C(C=C1c1ccc3c(c1)-c1cccc4cccc(c14)S3)c1ccccc1N2c1cccc(-c2ccccn2)n1 ; C1=CC2=CC(/C=C\C=C\Sc3ccc(-c4ccc5c(c4)c4ccccc4n5-c4nccc(-c5ccccc5)n4)cc32)C1]

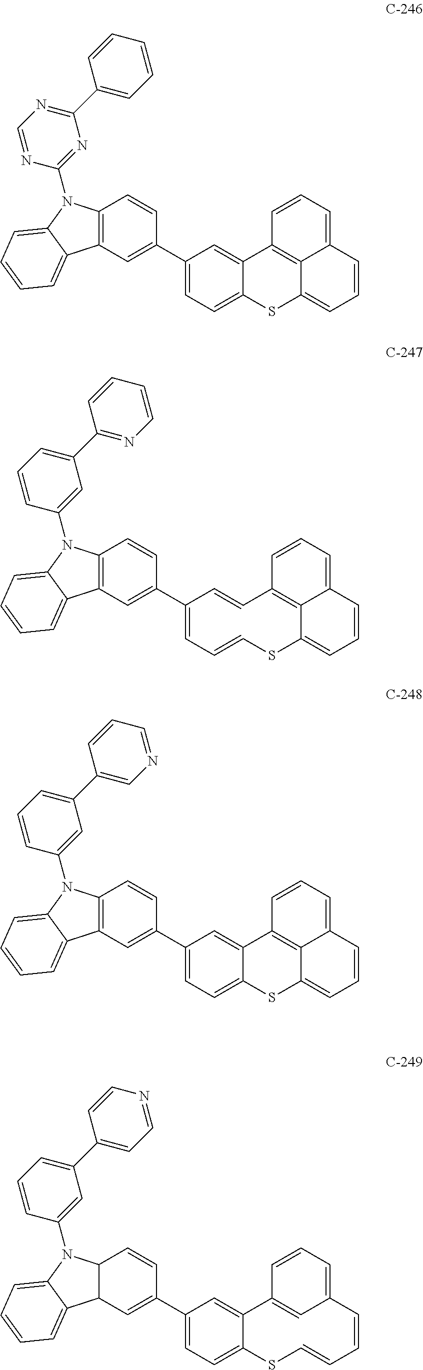 [SMILES: c1ccc(-c2ncnc(-n3c4ccccc4c4cc(-c5ccc6c(c5)-c5cccc7cccc(c57)S6)ccc43)n2)cc1 ; C1=C\Sc2cccc3cccc(c23)/C=C/C(c2ccc3c(c2)c2ccccc2n3-c2cccc(-c3ccccn3)c2)=C\1 ; c1cncc(-c2cccc(-n3c4ccccc4c4cc(-c5ccc6c(c5)-c5cccc7cccc(c57)S6)ccc43)c2)c1 ; C1=CC2C(C=C1c1ccc3c(c1)-c1cccc(c1)/C=C\C=C\S3)c1ccccc1N2c1cccc(-c2ccncc2)c1]

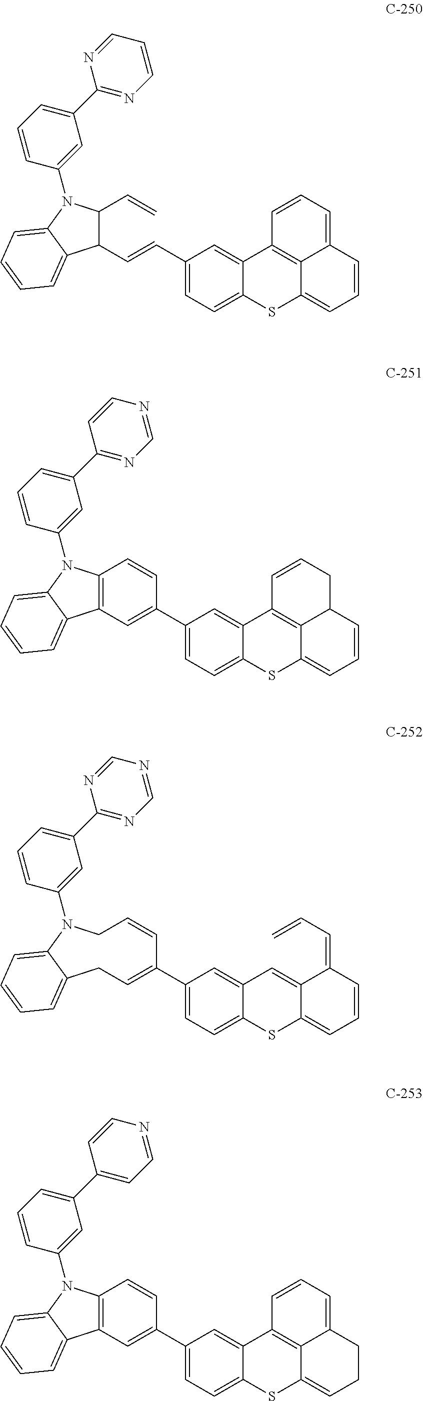 [SMILES: C=CC1C(/C=C/c2ccc3c(c2)-c2cccc4cccc(c24)S3)c2ccccc2N1c1cccc(-c2ncccn2)c1 ; C1=CC2CC=CC3=C2C(=C1)Sc1ccc(-c2ccc4c(c2)c2ccccc2n4-c2cccc(-c4ccncn4)c2)cc13 ; C=C/C=c1/cccc2c1=Cc1cc(C3=C/Cc4ccccc4N(c4cccc(-c5ncncn5)c4)C/C=C\3)ccc1S2 ; C1=C2Sc3ccc(-c4ccc5c(c4)c4ccccc4n5-c4cccc(-c5ccncc5)c4)cc3-c3cccc(c32)CC1]

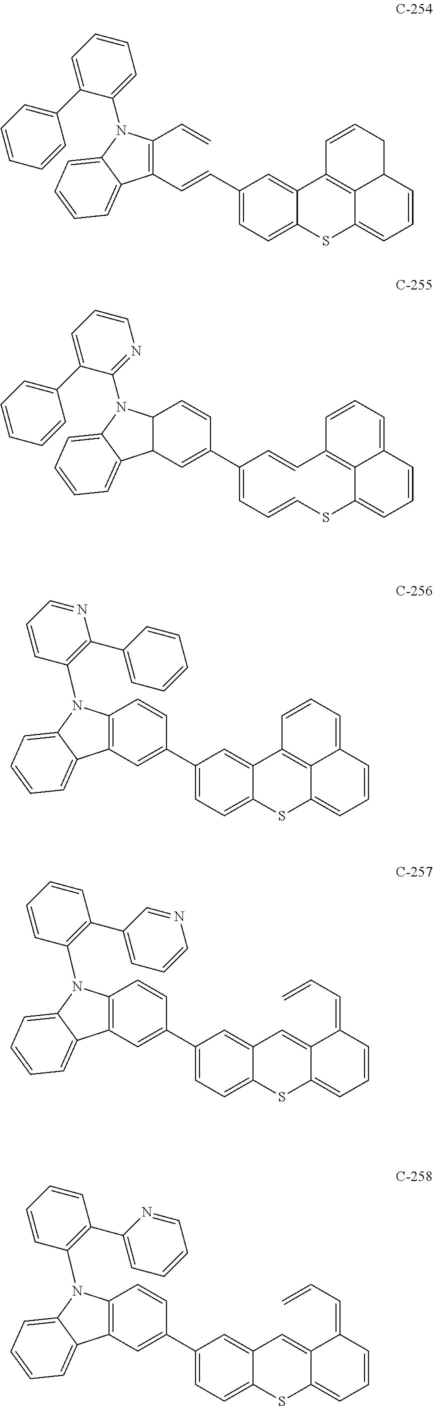 [SMILES: C=Cc1c(/C=C/c2ccc3c(c2)C2=C4C(=CC=CC4CC=C2)S3)c2ccccc2n1-c1ccccc1-c1ccccc1 ; C1=CC2C(C=C1C1=C/C=C/Sc3cccc4cccc(c34)\C=C\1)c1ccccc1N2c1ncccc1-c1ccccc1 ; c1ccc(-c2ncccc2-n2c3ccccc3c3cc(-c4ccc5c(c4)-c4cccc6cccc(c46)S5)ccc32)cc1 ; C=C/C=c1/cccc2c1=Cc1cc(-c3ccc4c(c3)c3ccccc3n4-c3ccccc3-c3cccnc3)ccc1S2 ; C=C/C=c1/cccc2c1=Cc1cc(-c3ccc4c(c3)c3ccccc3n4-c3ccccc3-c3ccccn3)ccc1S2]

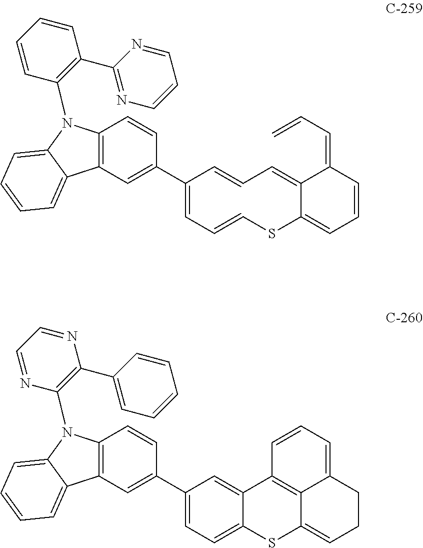 [SMILES: C=C/C=c1/cccc2/c1=C\C=C\C(c1ccc3c(c1)c1ccccc1n3-c1ccccc1-c1ncccn1)=C/C=C/S2 ; C1=C2Sc3ccc(-c4ccc5c(c4)c4ccccc4n5-c4nccnc4-c4ccccc4)cc3-c3cccc(c32)CC1]

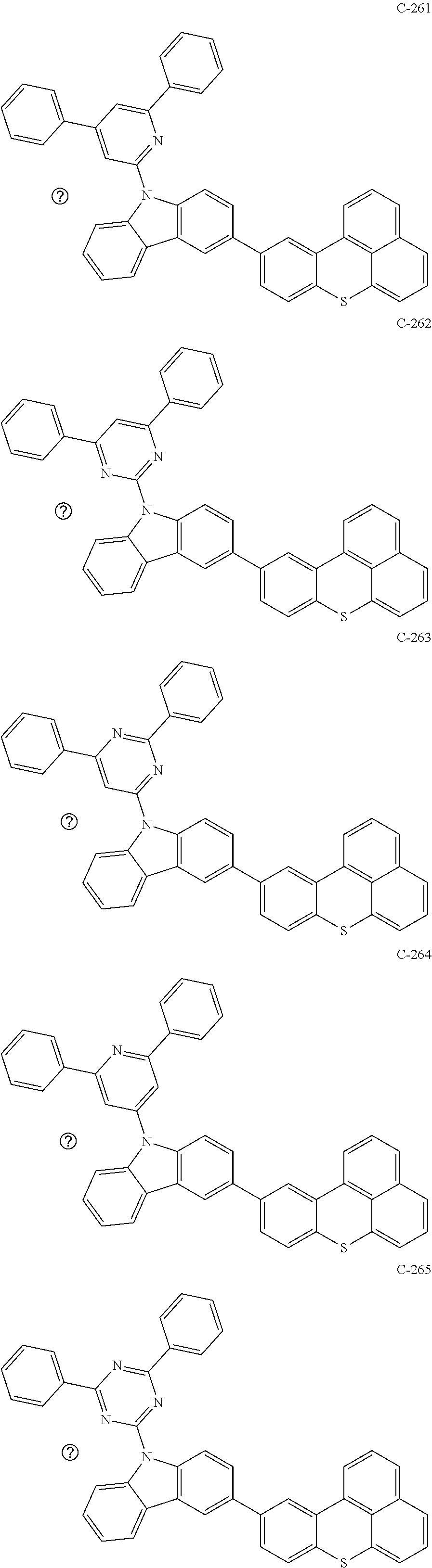 [SMILES: c1ccc(-c2cc(-c3ccccc3)nc(-n3c4ccccc4c4cc(-c5ccc6c(c5)-c5cccc7cccc(c57)S6)ccc43)c2)cc1 ; c1ccc(-c2cc(-c3ccccc3)nc(-n3c4ccccc4c4cc(-c5ccc6c(c5)-c5cccc7cccc(c57)S6)ccc43)n2)cc1 ; c1ccc(-c2cc(-n3c4ccccc4c4cc(-c5ccc6c(c5)-c5cccc7cccc(c57)S6)ccc43)nc(-c3ccccc3)n2)cc1 ; c1ccc(-c2cc(-n3c4ccccc4c4cc(-c5ccc6c(c5)-c5cccc7cccc(c57)S6)ccc43)cc(-c3ccccc3)n2)cc1 ; c1ccc(-c2nc(-c3ccccc3)nc(-n3c4ccccc4c4cc(-c5ccc6c(c5)-c5cccc7cccc(c57)S6)ccc43)n2)cc1]

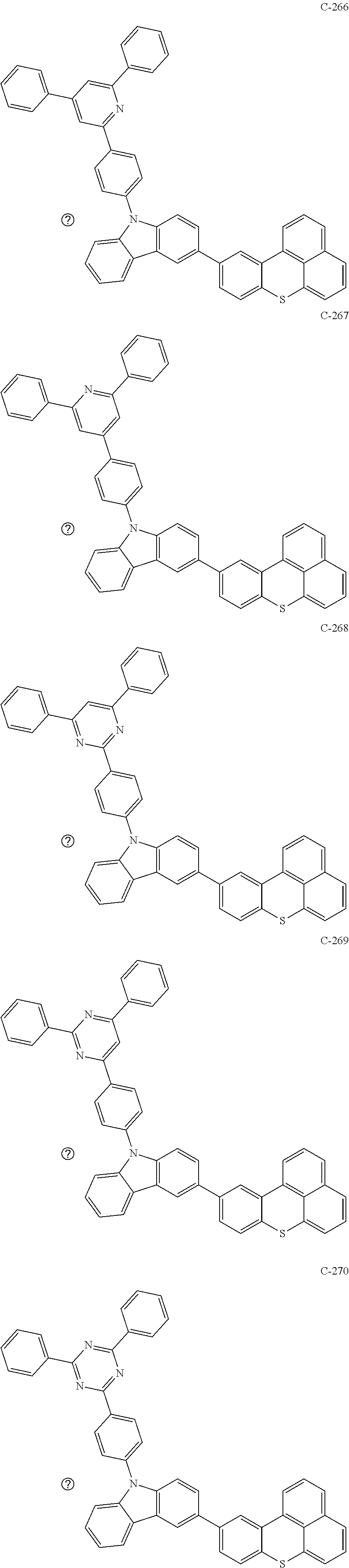 [SMILES: c1ccc(-c2cc(-c3ccccc3)nc(-c3ccc(-n4c5ccccc5c5cc(-c6ccc7c(c6)-c6cccc8cccc(c68)S7)ccc54)cc3)c2)cc1 ; c1ccc(-c2cc(-c3ccc(-n4c5ccccc5c5cc(-c6ccc7c(c6)-c6cccc8cccc(c68)S7)ccc54)cc3)cc(-c3ccccc3)n2)cc1 ; c1ccc(-c2cc(-c3ccccc3)nc(-c3ccc(-n4c5ccccc5c5cc(-c6ccc7c(c6)-c6cccc8cccc(c68)S7)ccc54)cc3)n2)cc1 ; c1ccc(-c2cc(-c3ccc(-n4c5ccccc5c5cc(-c6ccc7c(c6)-c6cccc8cccc(c68)S7)ccc54)cc3)nc(-c3ccccc3)n2)cc1 ; c1ccc(-c2nc(-c3ccccc3)nc(-c3ccc(-n4c5ccccc5c5cc(-c6ccc7c(c6)-c6cccc8cccc(c68)S7)ccc54)cc3)n2)cc1]

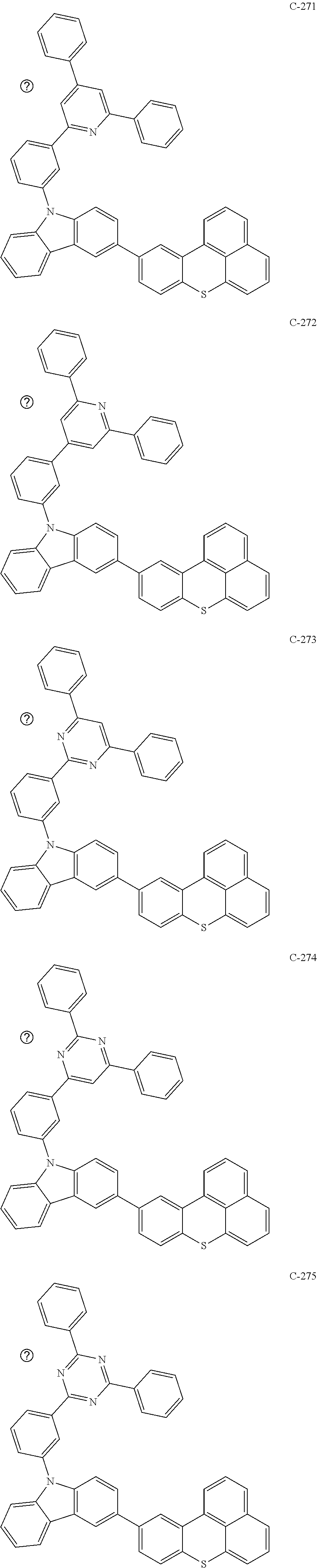 [SMILES: c1ccc(-c2cc(-c3ccccc3)nc(-c3cccc(-n4c5ccccc5c5cc(-c6ccc7c(c6)-c6cccc8cccc(c68)S7)ccc54)c3)c2)cc1 ; c1ccc(-c2cc(-c3cccc(-n4c5ccccc5c5cc(-c6ccc7c(c6)-c6cccc8cccc(c68)S7)ccc54)c3)cc(-c3ccccc3)n2)cc1 ; c1ccc(-c2cc(-c3ccccc3)nc(-c3cccc(-n4c5ccccc5c5cc(-c6ccc7c(c6)-c6cccc8cccc(c68)S7)ccc54)c3)n2)cc1 ; c1ccc(-c2cc(-c3cccc(-n4c5ccccc5c5cc(-c6ccc7c(c6)-c6cccc8cccc(c68)S7)ccc54)c3)nc(-c3ccccc3)n2)cc1 ; c1ccc(-c2nc(-c3ccccc3)nc(-c3cccc(-n4c5ccccc5c5cc(-c6ccc7c(c6)-c6cccc8cccc(c68)S7)ccc54)c3)n2)cc1]

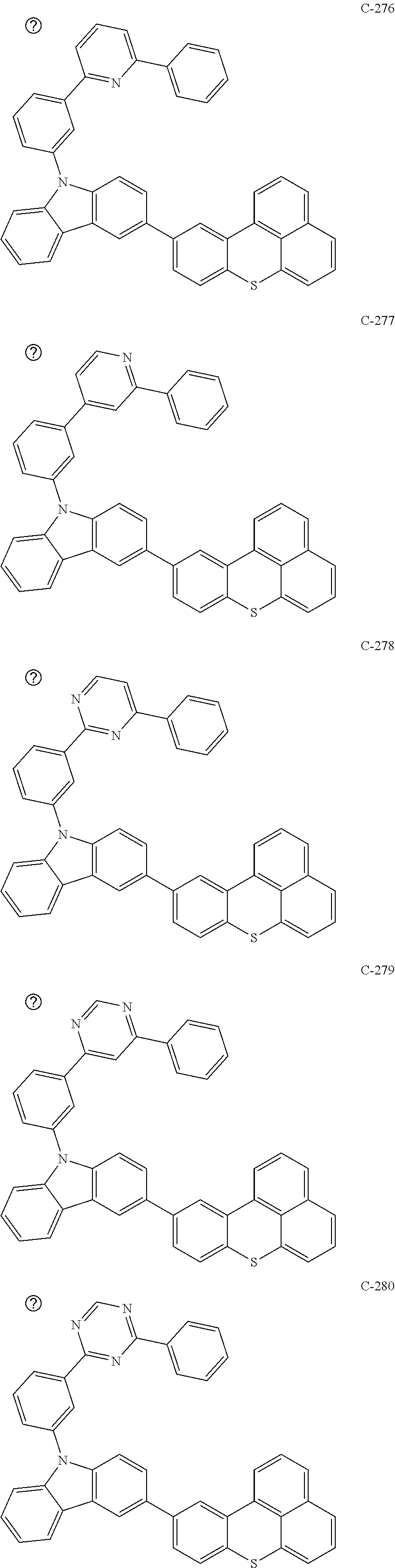 [SMILES: c1ccc(-c2cccc(-c3cccc(-n4c5ccccc5c5cc(-c6ccc7c(c6)-c6cccc8cccc(c68)S7)ccc54)c3)n2)cc1 ; c1ccc(-c2cc(-c3cccc(-n4c5ccccc5c5cc(-c6ccc7c(c6)-c6cccc8cccc(c68)S7)ccc54)c3)ccn2)cc1 ; c1ccc(-c2ccnc(-c3cccc(-n4c5ccccc5c5cc(-c6ccc7c(c6)-c6cccc8cccc(c68)S7)ccc54)c3)n2)cc1 ; c1ccc(-c2cc(-c3cccc(-n4c5ccccc5c5cc(-c6ccc7c(c6)-c6cccc8cccc(c68)S7)ccc54)c3)ncn2)cc1 ; c1ccc(-c2ncnc(-c3cccc(-n4c5ccccc5c5cc(-c6ccc7c(c6)-c6cccc8cccc(c68)S7)ccc54)c3)n2)cc1]

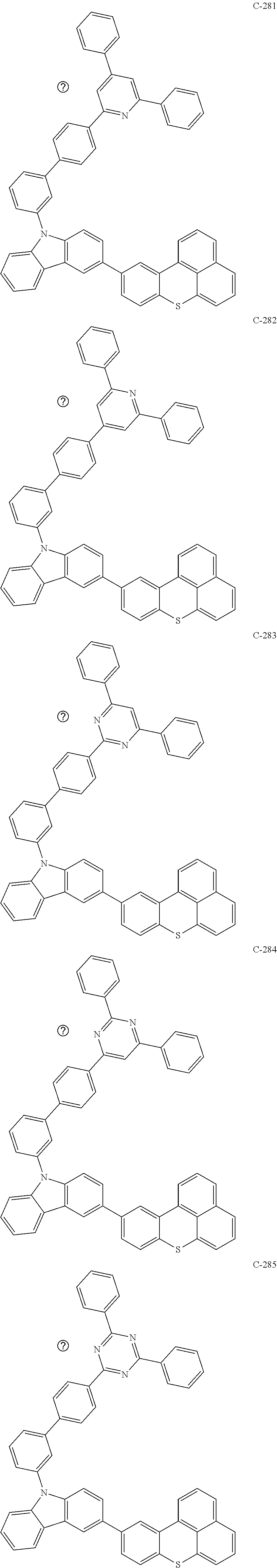

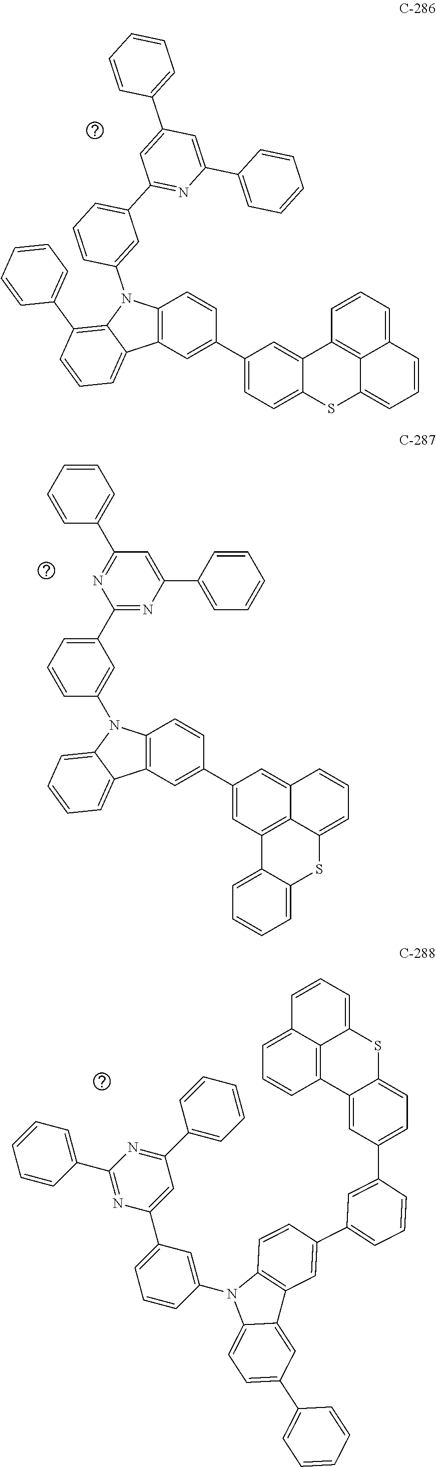 [SMILES: c1ccc(-c2cc(-c3ccccc3)nc(-c3cccc(-n4c5ccc(-c6ccc7c(c6)-c6cccc8cccc(c68)S7)cc5c5cccc(-c6ccccc6)c54)c3)c2)cc1 ; c1ccc(-c2cc(-c3ccccc3)nc(-c3cccc(-n4c5ccccc5c5cc(-c6cc7c8c(cccc8c6)Sc6ccccc6-7)ccc54)c3)n2)cc1 ; c1ccc(-c2ccc3c(c2)c2cc(-c4cccc(-c5ccc6c(c5)-c5cccc7cccc(c57)S6)c4)ccc2n3-c2cccc(-c3cc(-c4ccccc4)nc(-c4ccccc4)n3)c2)cc1]

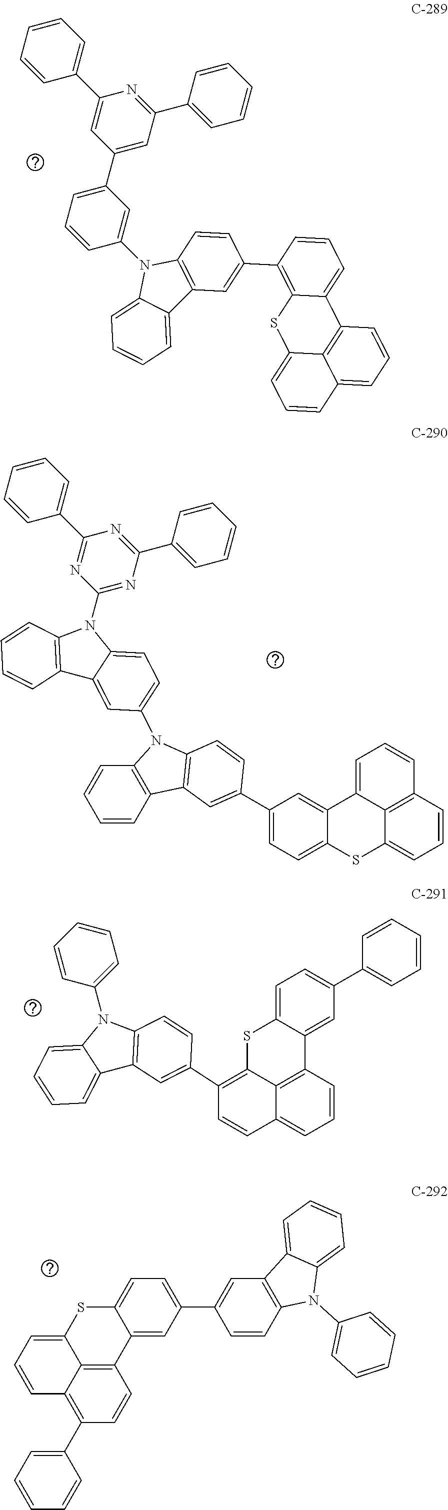 [SMILES: c1ccc(-c2cc(-c3cccc(-n4c5ccccc5c5cc(-c6cccc7c6Sc6cccc8cccc-7c68)ccc54)c3)cc(-c3ccccc3)n2)cc1 ; c1ccc(-c2nc(-c3ccccc3)nc(-n3c4ccccc4c4cc(-n5c6ccccc6c6cc(-c7ccc8c(c7)-c7cccc9cccc(c79)S8)ccc65)ccc43)n2)cc1 ; c1ccc(-c2ccc3c(c2)-c2cccc4ccc(-c5ccc6c(c5)c5ccccc5n6-c5ccccc5)c(c24)S3)cc1 ; c1ccc(-c2ccc3c4c(cccc24)Sc2ccc(-c4ccc5c(c4)c4ccccc4n5-c4ccccc4)cc2-3)cc1]

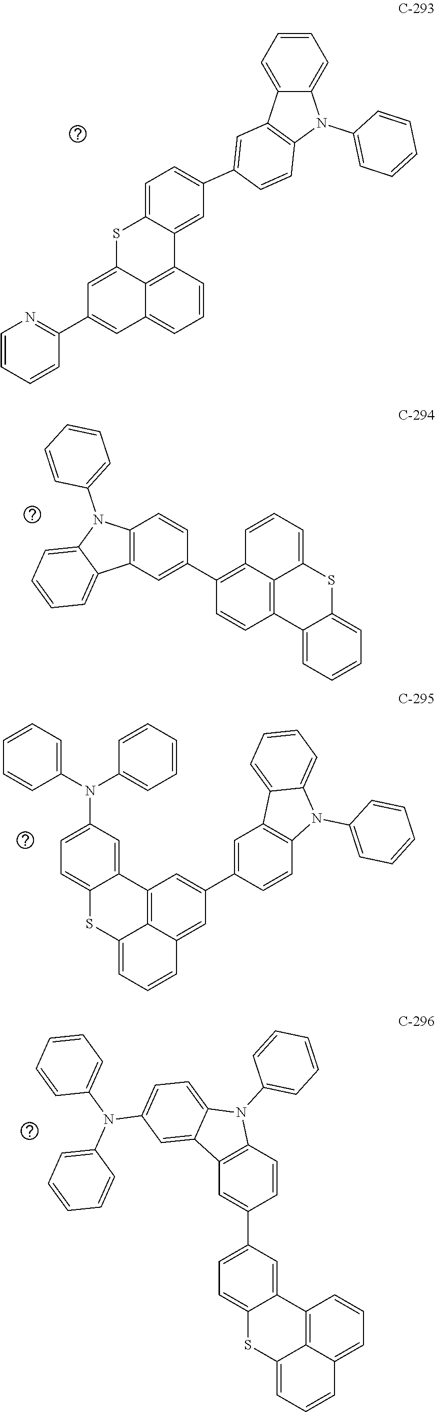 [SMILES: c1ccc(-n2c3ccccc3c3cc(-c4ccc5c(c4)-c4cccc6cc(-c7ccccn7)cc(c46)S5)ccc32)cc1 ; c1ccc(-n2c3ccccc3c3cc(-c4ccc5c6c(cccc46)Sc4ccccc4-5)ccc32)cc1 ; c1ccc(N(c2ccccc2)c2ccc3c(c2)-c2cc(-c4ccc5c(c4)c4ccccc4n5-c4ccccc4)cc4cccc(c24)S3)cc1 ; c1ccc(N(c2ccccc2)c2ccc3c(c2)c2cc(-c4ccc5c(c4)-c4cccc6cccc(c46)S5)ccc2n3-c2ccccc2)cc1]

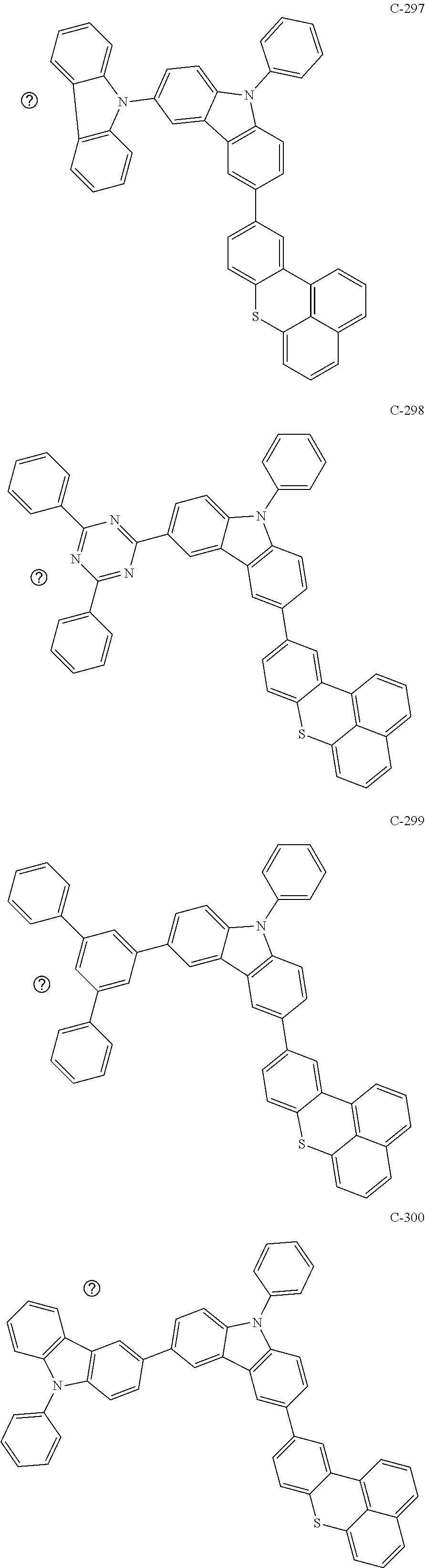 [SMILES: c1ccc(-n2c3ccc(-c4ccc5c(c4)-c4cccc6cccc(c46)S5)cc3c3cc(-n4c5ccccc5c5ccccc54)ccc32)cc1 ; c1ccc(-c2nc(-c3ccccc3)nc(-c3ccc4c(c3)c3cc(-c5ccc6c(c5)-c5cccc7cccc(c57)S6)ccc3n4-c3ccccc3)n2)cc1 ; c1ccc(-c2cc(-c3ccccc3)cc(-c3ccc4c(c3)c3cc(-c5ccc6c(c5)-c5cccc7cccc(c57)S6)ccc3n4-c3ccccc3)c2)cc1 ; c1ccc(-n2c3ccccc3c3cc(-c4ccc5c(c4)c4cc(-c6ccc7c(c6)-c6cccc8cccc(c68)S7)ccc4n5-c4ccccc4)ccc32)cc1]

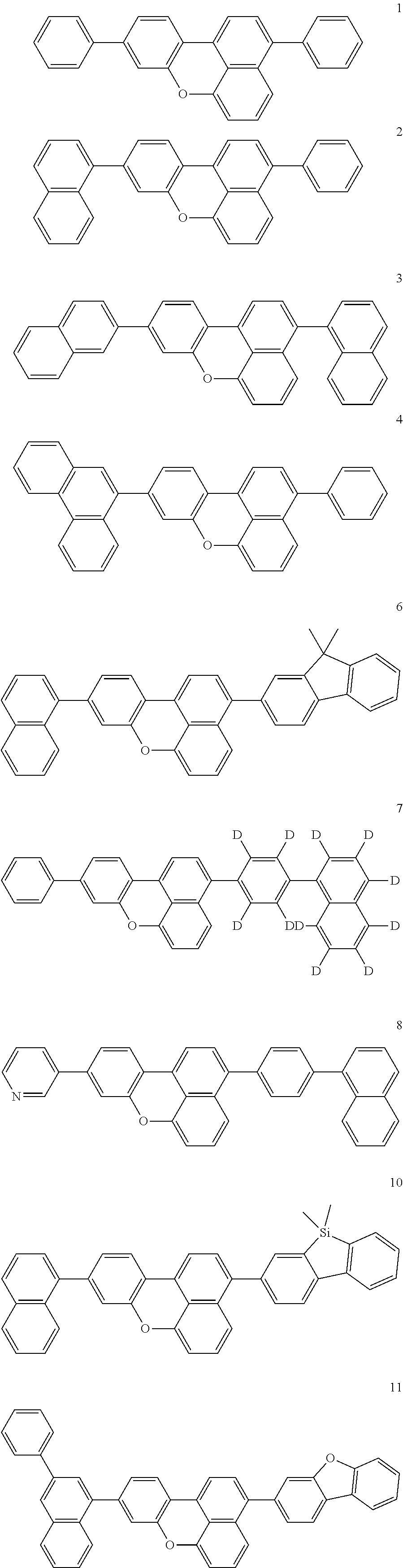

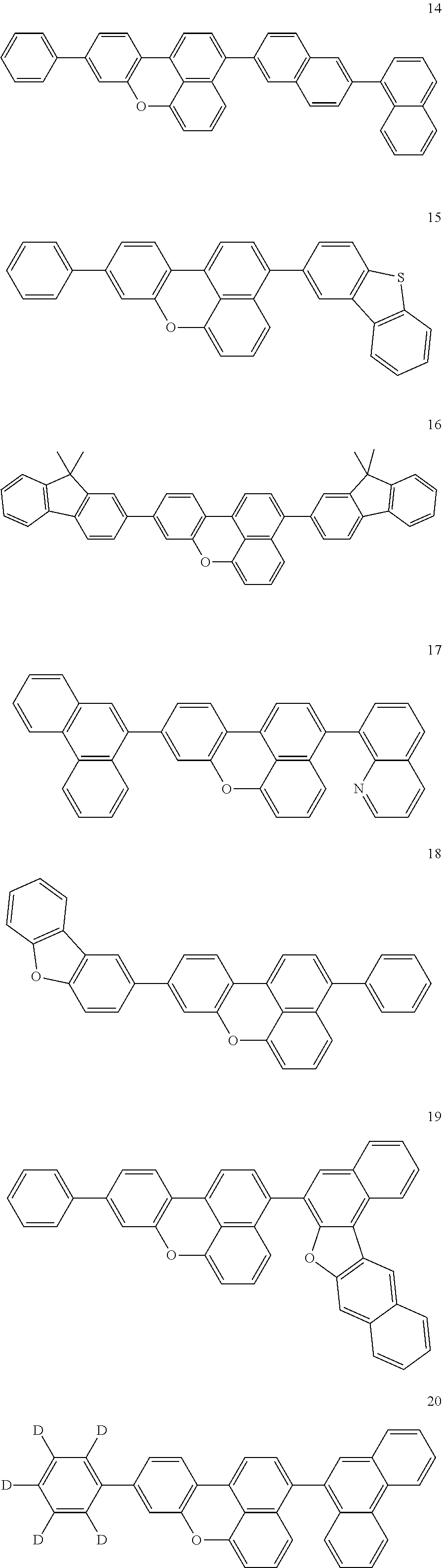

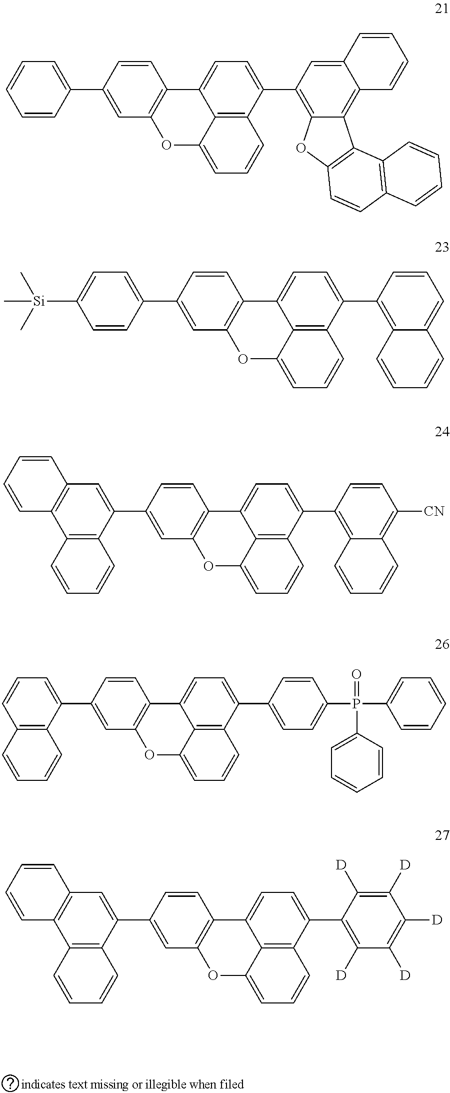 [SMILES: c1ccc(-c2ccc3c(c2)Oc2cccc4c(-c5cc6ccccc6c6c5oc5ccc7ccccc7c56)ccc-3c24)cc1 ; C[Si](C)(C)c1ccc(-c2ccc3c(c2)Oc2cccc4c(-c5cccc6ccccc56)ccc-3c24)cc1 ; N#Cc1ccc(-c2ccc3c4c(cccc24)Oc2cc(-c4cc5ccccc5c5ccccc45)ccc2-3)c2ccccc12 ; O=P(c1ccccc1)(c1ccccc1)c1ccc(-c2ccc3c4c(cccc24)Oc2cc(-c4cccc5ccccc45)ccc2-3)cc1 ; [2H]c1c([2H])c([2H])c(-c2ccc3c4c(cccc24)Oc2cc(-c4cc5ccccc5c5ccccc45)ccc2-3)c([2H])c1[2H]]

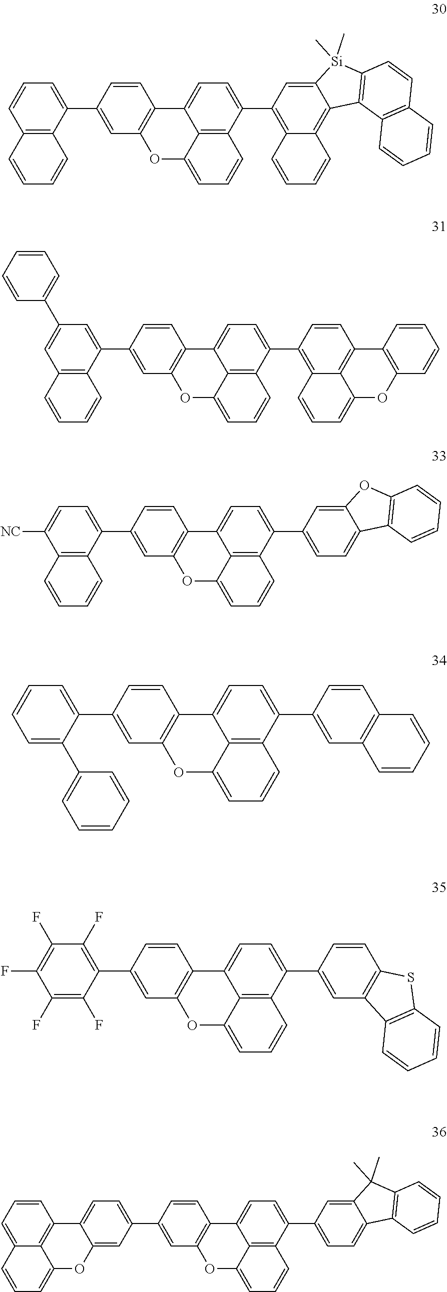 [SMILES: C[Si]1(C)c2ccc3ccccc3c2-c2c1cc(-c1ccc3c4c(cccc14)Oc1cc(-c4cccc5ccccc45)ccc1-3)c1ccccc21 ; c1ccc(-c2cc(-c3ccc4c(c3)Oc3cccc5c(-c6ccc7c8c(cccc68)Oc6ccccc6-7)ccc-4c35)c3ccccc3c2)cc1 ; N#Cc1ccc(-c2ccc3c(c2)Oc2cccc4c(-c5ccc6c(c5)oc5ccccc56)ccc-3c24)c2ccccc12 ; c1ccc(-c2ccccc2-c2ccc3c(c2)Oc2cccc4c(-c5ccc6ccccc6c5)ccc-3c24)cc1 ; Fc1c(F)c(F)c(-c2ccc3c(c2)Oc2cccc4c(-c5ccc6sc7ccccc7c6c5)ccc-3c24)c(F)c1F ; CC1(C)c2ccccc2-c2ccc(-c3ccc4c5c(cccc35)Oc3cc(-c5ccc6c(c5)Oc5cccc7cccc-6c57)ccc3-4)cc21]

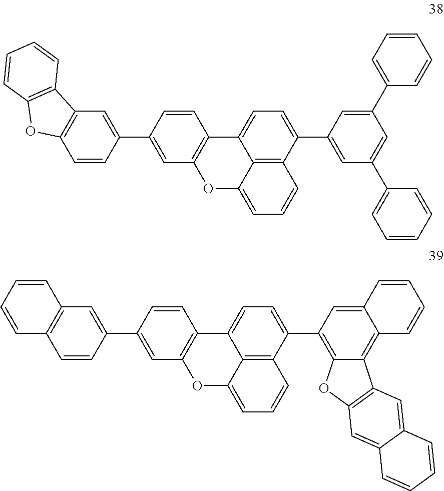

### <Layer Structure of Organic EL Device>

The organic EL device according to the embodiment of the present invention includes an anode, a cathode, and an organic layer intervening between the anode and the cathode, and the organic layer includes a light emitting layer.

In the first organic EL device, at least one layer of the organic layer contains the inventive compound. As described above, the inventive compound is contained in a hole transporting zone (a hole injecting layer, a hole transporting layer, an electron blocking layer, an exciton blocking layer), a light emitting layer, a space layer, or an electron transporting zone (an electron injecting layer, an electron transporting layer, a hole blocking layer) provided between a cathode and a light emitting layer.

Further, as described above, the second organic EL device includes an anode, a hole transporting zone, a light emitting layer, and a cathode in this order, and the hole transporting zone contains the compound A satisfying the above-described conditions (A) to (C). In addition, other materials and device configurations known in the related art can be applied to the organic EL device as long as the effects of the present invention are not impaired.

The hole transporting zone is composed of at least one layer having a hole transporting function. Examples of the layer constituting the hole transporting zone include a hole injecting layer, a hole transporting layer, an electron blocking layer, and an exciton blocking layer. The hole transporting zone may be composed of a plurality of layers or may be a single layer.

In a preferred embodiment of the second organic EL device, the hole transporting zone is composed of a plurality of layers including a hole transporting layer, or is composed of a single hole transporting layer, and the compound A is contained in the hole transporting layer. In other words, the compound A is preferably used as a material for the hole transporting layer.

In one embodiment of the present invention, the hole transporting zone includes a hole transporting layer and a second layer other than the hole transporting layer. In this case, the second layer may or may not contain the inventive compound. In addition, the second layer may contain the compound A or may not contain the compound A. The second layer may be disposed between the anode and the hole transporting layer or between the hole transporting layer and the light emitting layer.

In a preferred embodiment of the present invention, as described below, the hole transporting layer is a multilayer structure including two or more layers, and the hole transporting layer has a two layer structure including a first hole transporting layer (on the anode side) and a second hole transporting layer (on the cathode side). In this case, the inventive compound may be contained only in the first hole transporting layer, may be contained only in the second hole transporting layer, or may be contained in the first and second hole transporting layers. Further, the compound A may be contained only in the first hole transporting layer, may be contained only in the second hole transporting layer, or may be contained in the first and second hole transporting layers.

The organic EL device may include an organic layer other than the hole transporting zone and the light emitting layer, and at least one of the inventive compound and the compound A may be contained in the other organic layer.

Examples of the organic layer containing at least one of the inventive compound and the compound A include, but are not limited to, a space layer and an electron transporting zone (an electron injecting layer, an electron transporting layer, or a hole blocking layer) provided between the cathode and the light emitting layer.

The light emitting layer may contain at least one of the inventive compound and the compound A.

In one embodiment of the present invention, the hole transporting layer constituting the hole transporting zone has a two layer structure including a first hole transporting layer (on the anode side) and a second hole transporting layer (on the cathode side), and no other layer is included between the second hole transporting layer and the light emitting layer. In other words, in one embodiment of the present invention, the second hole transporting layer and the light emitting layer are in direct contact with each other.

The compound A is preferably used as a material for a hole transporting zone of a fluorescent or phosphorescent EL device, more preferably as a material for a hole injecting layer, a hole transporting layer, an electron blocking layer, or an exciton blocking layer, and particularly preferably as a material for a hole injecting layer or a hole transporting layer.

In one embodiment of the present invention, the light emitting layer contains a fluorescent dopant material.

In one embodiment of the present invention, the light emitting layer contains a phosphorescent dopant material.

Hereinafter, members used in the first and second organic EL devices and materials constituting each layer other than the inventive compound and the compound A will be described.

### (Substrate)

The substrate is used as a support of the organic EL device. As the substrate, for example, a plate of glass, quartz, or plastic can be used. Alternatively, a flexible substrate may be used. Examples of the flexible substrate include a plastic substrate made of polycarbonate, polyarylate, polyethersulfone, polypropylene, polyester, polyvinyl fluoride, or polyvinyl chloride. In addition, an inorganic vapor deposition film can be used.

### (Anode)

It is preferred that a metal, an alloy, an electrically conductive compound, or a mixture thereof which has a high work function (specifically 4.0 eV or more) is used for the anode formed on the substrate. Specific examples thereof include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. Besides, examples thereof include gold (Au), platinum (Pt), nickel (Ni), tungsten (W), chromium (Cr), molybdenum (Mo), iron (Fe), cobalt (Co), copper (Cu), palladium (Pd), titanium (Ti), or nitrides of the metals (for example, titanium nitride).

These materials are usually deposited by a sputtering method. For example, through a sputtering method, it is possible to form indium oxide-zinc oxide by using a target in which 1 to 10 wt % of zinc oxide is added to indium oxide, and to form indium oxide containing tungsten oxide and zinc oxide by using a target containing 0.5 to 5 wt % of tungsten oxide and 0.1 to 1 wt % of zinc oxide with respect to indium oxide. Besides, the manufacturing may be performed by a vacuum vapor deposition method, a coating method, an inkjet method, or a spin coating method.

### (Hole Transporting Zone)

As described above, the organic layer may include a hole transporting zone between the anode and the light emitting layer. The hole transporting zone is composed of a hole injecting layer, a hole transporting layer, and an electron blocking layer. It is preferable that the hole transporting zone contains at least one of the inventive compound and the compound A. It is preferable that at least one of the inventive compound and the compound A is contained in at least one layer of the layers constituting the hole transporting layer, and it is more preferable that at least one of the inventive compound and the compound A is contained in the hole transporting layer.

The hole injecting layer formed in contact with the anode is formed by using a material that facilitates hole injection regardless of a work function of the anode, and thus, it is possible to use materials generally used as an electrode material (for example, metals, alloys, electrically conductive compounds, or mixtures thereof, elements belonging to Group 1 or Group 2 of the periodic table of the elements).

It is also possible to use elements belonging to Group 1 or Group 2 of the periodic table of the elements, that is, alkali metals such as lithium (Li) and cesium (Cs), alkaline earth metals such as magnesium (Mg), calcium (Ca), and strontium (Sr) and alloys containing these (such as MgAg, and AlLi), and rare earth metals such as europium (Eu), and ytterbium (Yb) and alloys containing these, which are materials having a small work function. When the anode is formed by using the alkali metals, the alkaline earth metals, and the alloys containing these, a vacuum vapor deposition method or a sputtering method can be adopted. Further, when a silver paste is used, a coating method or an inkjet method can be adopted.

### (Hole Injecting Layer)

The hole injecting layer is a layer containing a material having a high hole injection capability (a hole injecting material) and is provided between the anode and the light emitting layer, or between the hole transporting layer, if exists, and the anode.

As the hole injecting material except the inventive compound, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, and the like can be used.

Examples of the hole injecting layer material also include aromatic amine compounds as low-molecular weight organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

High-molecular weight compounds (such as oligomers, dendrimers, and polymers) may also be used. Examples thereof include high-molecular weight compounds, such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). In addition, high-molecular weight compounds to which an acid is added, such as poly(3,4-ethylenedioxythiophene)/poly(styrene sulfonic acid) (PEDOT/PSS), and polyaniline/poly(styrenesulfonic acid) (PAni/PSS), can also be used.

Furthermore, it is also preferred to use an acceptor material, such as a hexaazatriphenylene (HAT) compound represented by formula (K).

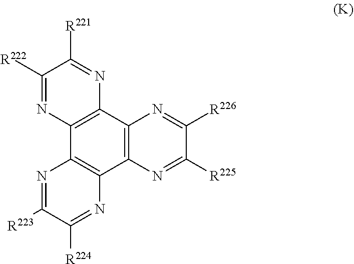

In the aforementioned formula, R²²¹ to R²²⁶ each independently represent a cyano group, —CONH₂, a carboxy group, or —COOR²²⁷ (R²²⁷ represents an alkyl group having 1 to 20 carbon atoms or a cycloalkyl group having 3 to 20 carbon atoms). In addition, adjacent two selected from R²²¹ and R²²², R²²³ and R²²⁴, and R²²⁵ and R²²⁶ may be bonded to each other to form a group represented by —CO—O—CO—.

Examples of R²²⁷ include a methyl group, an ethyl group, a n-propyl group, an isopropyl group, a n-butyl group, an isobutyl group, a t-butyl group, a cyclopentyl group, and a cyclohexyl group.

### (Hole Transporting Layer)

The hole transporting layer is a layer containing a material having a high hole transporting capability (a hole transporting material) and is provided between the anode and the light emitting layer, or between the hole injecting layer, if exists, and the light emitting layer. The inventive compound can be used as the hole transporting layer either singly or as combined with the compound mentioned below.

The hole transporting layer may have a single layer structure or a multilayer structure including two or more layers. For example, the hole transporting layer may have a two layer structure including a first hole transporting layer (on the anode side) and a second hole transporting layer (on the cathode side). That is, the hole transporting zone may include the first hole transporting layer on the anode side and the second hole transporting layer on the cathode side. In addition, the hole transporting layer may have a three layer structure including a first hole transporting layer, a second hole transporting layer, and a third hole transporting layer in this order from the anode side. That is, the third hole transporting layer may be disposed between the second hole transporting layer and the light emitting layer.

In one embodiment of the present invention, the hole transporting layer having a single layer structure is preferably disposed adjacent to the light emitting layer, and the hole transporting layer that is closest to the cathode in the multilayer structure, such as the second hole transporting layer of the two layer structure or the third hole transporting layer of the three layer structure, is preferably disposed adjacent to the light emitting layer. In another embodiment of the present invention, an electron blocking layer described later may be interposed between the hole transporting layer having a single layer structure and the light emitting layer, or between the hole transporting layer that is closest to the light emitting layer in the multilayer structure and the light emitting layer.

In one embodiment of the organic electroluminescent device according to the present invention, at least one of the first hole transporting layer and the second hole transporting layer contains at least one of the inventive compound and the compound A. Specifically, in the hole transporting layer having a two layer structure, at least one of the inventive compound and the compound A may be contained in either or both of the first hole transporting layer and the second hole transporting layer. In another embodiment, at least one of the first to third hole transporting layers contains at least one of the inventive compound and the compound A. Specifically, in the hole transporting layer having a three layer structure, at least one of the inventive compound and the compound A may be contained in only one of the first to third hole transporting layers, in any two of the first to third hole transporting layers, or in all of the first to third hole transporting layers.

In one embodiment of the present invention, at least one of the inventive compound and the compound A is preferably contained in the second hole transporting layer, and specifically, at least one of the inventive compound and the compound A is preferably contained only in the second hole transporting layer, or at least one of the inventive compound and the compound A is preferably contained in the first hole transporting layer and the second hole transporting layer.

In one embodiment of the present invention, the inventive compound or the compound A contained in one or both of the first hole transporting layer and the second hole transporting layer, or the inventive compound or the compound A contained in at least one or more of the first to third hole transporting layers is preferably a protium compound from the viewpoint of production cost.

The protium compound is an inventive compound in which all hydrogen atoms in the inventive compound are protium atoms.

Therefore, the present invention includes an organic EL device including at least one of the inventive compound and the compound A in which one or both of the first hole transporting layer and the second hole transporting layer or at least one or more of the first to third hole transporting layers are substantially composed only of a protium compound. The expression “the inventive compound substantially composed of only a protium compound” means that the content ratio of the protium compound to the total amount of the inventive compound is 90 mol % or more, preferably 95 mol % or more, and more preferably 99 mol % or more (each including 100%). The same applies to the expression “the compound A substantially composed of only a protium compound”.

As the hole transporting layer material except the inventive compound and the compound A, for example, an aromatic amine compound, a carbazole derivative, and an anthracene derivative can be used.

Examples of the aromatic amine compound include 4,4′-bis[N-(1-naphthyl)-N-phenylamino]biphenyl (abbreviation: NPB) or N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB). The aforementioned compounds have a hole mobility of 106 cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

High-molecular weight compounds, such as poly(N-vinylcarbazole) (abbreviation: PVK) and poly(4-vinyltriphenylamine) (abbreviation: PVTPA), can also be used.

However, compounds other than those as mentioned above can also be used so long as they are compounds high in the hole transporting capability rather than in the electron transporting capability.

In one embodiment of the organic EL device according to the present invention, the first hole transporting layer contains a compound represented by the following formula (21) or formula (22).

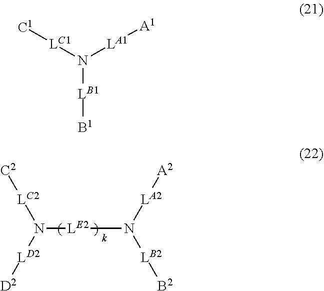

In the formula (21) and the formula (22),
L^{A1}, L^{B1}, L^{C1}, L^{A2}, L^{B2}, L^{C2}, and L^{D2} are each independently a single bond, a substituted or unsubstituted arylene group having 6 to 50 ring carbon atoms, or a substituted or unsubstituted divalent heterocyclic group having 5 to 50 ring atoms;
k is 1, 2, 3, or 4;
in the case where k is 1, L^{E2} is a substituted or unsubstituted arylene group having 6 to 50 ring carbon atoms, or a substituted or unsubstituted divalent heterocyclic group having 5 to 50 ring atoms;
in the case where k is 2, 3, or 4, a plurality of L^{E2}'s are the same as or different from each other;
in the case where k is 2, 3, or 4, a plurality of L^{E2}'s are bonded to each other to form a substituted or unsubstituted monocyclic ring, are bonded to each other to form a substituted or unsubstituted condensed ring, or are not bonded to each other;
L^{E2} which does not form a monocyclic ring and does not form a condensed ring is a substituted or unsubstituted arylene group having 6 to 50 ring carbon atoms, or a substituted or unsubstituted divalent heterocyclic group having 5 to 50 ring atoms;
A¹, B¹, C¹, A², B², C², and D² are each independently a substituted or unsubstituted aryl group having 6 to 50 ring carbon atoms, a substituted or unsubstituted heterocyclic group having 5 to 50 ring atoms, or —Si(R′₉₀₁)(R′₉₀₂)(R′₉₀₃); R′₉₀₁, R′₉₀₂, and R′₉₀₃ are each independently a substituted or unsubstituted aryl group having 6 to 50 ring carbon atoms;
in the case where a plurality of R′₉₀₁'s exist, the plurality of R′₉₀₁'s are the same as or different from each other;
in the case where a plurality of R′₉₀₂'s exist, the plurality of R′₉₀₂'s are the same as or different from each other;
in the case where a plurality of R′₉₀₃'s exist, the plurality of R′₉₀₃'s are the same as or different from each other.

R₉₀₁ to R₉₀₇ are each independently a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms;
in the case where a plurality of R₉₀₁'s are present, the plurality of R₉₀₁'s are the same as or different from each other,
in the case where a plurality of R₉₀₂'s are present, the plurality of R₉₀₂'s are the same as or different from each other,
in the case where a plurality of R₉₀₃'s are present, the plurality of R₉₀₃'s are the same as or different from each other,
in the case where a plurality of R₉₀₄'s are present, the plurality of R₉₀₄'s are the same as or different from each other,
in the case where a plurality of R₉₀₅'s are present, the plurality of R₉₀₅'s are the same as or different from each other,
in the case where a plurality of R₉₀₆'s are present, the plurality of R₉₀₆'s are the same as or different from each other, and
in the case where a plurality of R₉₀₇'s are present, the plurality of R₉₀₇'s are the same as or different from each other.

The first hole transporting layer may contain one kind of the compounds represented by the formula (21) and the formula (22), or may contain a plurality of kinds of the compounds represented by the formula (21) and the formula (22).

In the formula (21) and the formula (22), A1, B1, C1, A2, B2, C2, and D2 are preferably each independently selected from substituted or unsubstituted phenylene groups, substituted or unsubstituted biphenyl groups, substituted or unsubstituted terphenyl groups, substituted or unsubstituted naphthyl groups, substituted or unsubstituted fluorenyl groups, substituted or unsubstituted dibenzofuranyl groups, substituted or unsubstituted dibenzothiophenyl groups, and substituted or unsubstituted carbazolyl groups.

More preferably, at least one of A1, B1, and C1 in the formula (21), and at least one of A2, B2, C2, and D2 in the formula (22) is a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, or a substituted or unsubstituted carbazolyl group.

The fluorenyl group that can be taken by A1, B1, C1, A2, B2, C2, and D2 may have a substituent at the 9-position, and may be, for example, a 9,9-dimethylfluorenyl group or a 9,9-diphenylfluorenyl group. The substituents at the 9-position may form a ring, and for example, the substituents at the 9-position may form a fluorene skeleton or a xanthene skeleton.

L^{A1}, L^{B1}, L^{C1}, L^{A2}, L^{B2}, L^{C2}, and L^{D2} are preferably each independently a single bond or a substituted or unsubstituted arylene group having 6 to 12 ring carbon atoms.

Specific examples of the compounds represented by the formula (21) and the formula (22) include the following compounds.

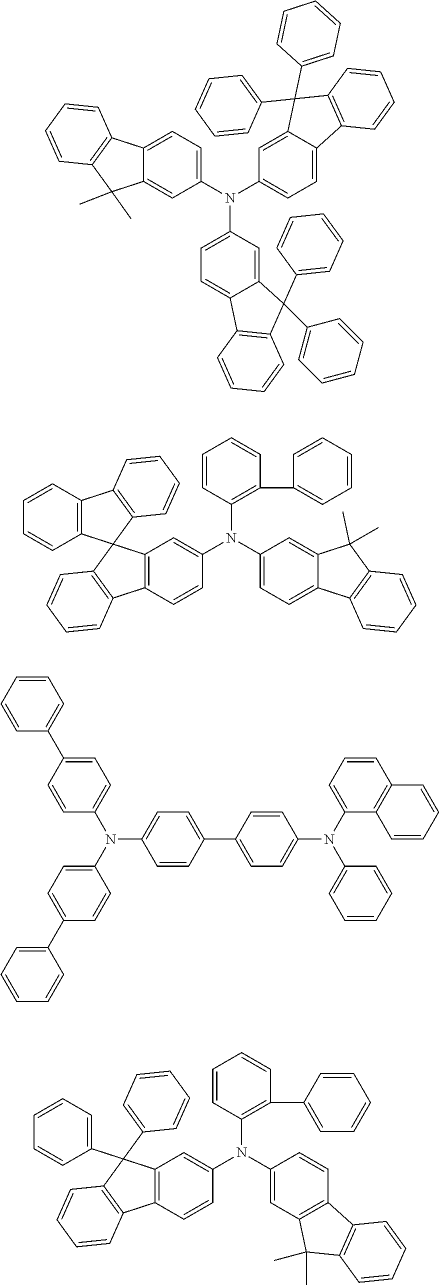 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc4c(c3)C(c3ccccc3)(c3ccccc3)c3ccccc3-4)c3ccccc3-c3ccccc3)cc21]

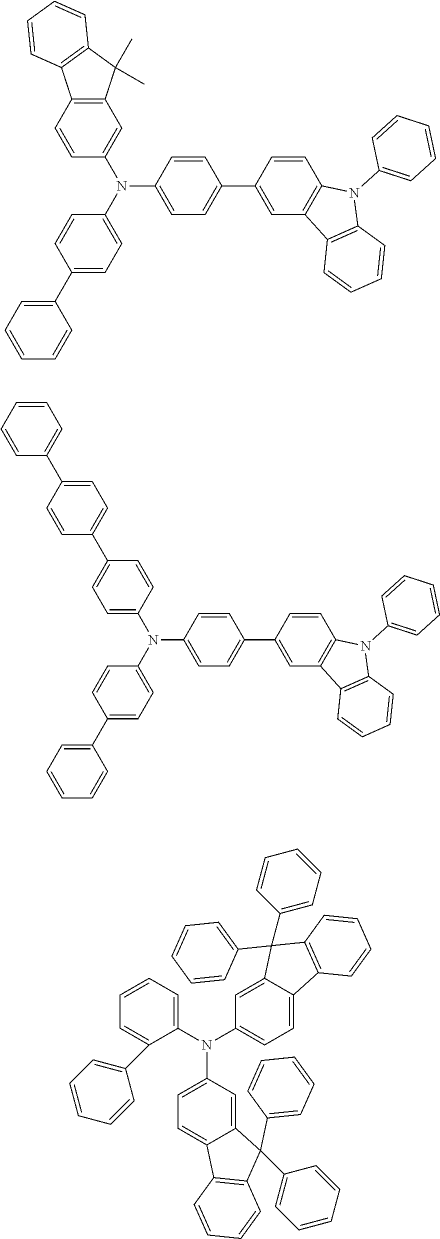

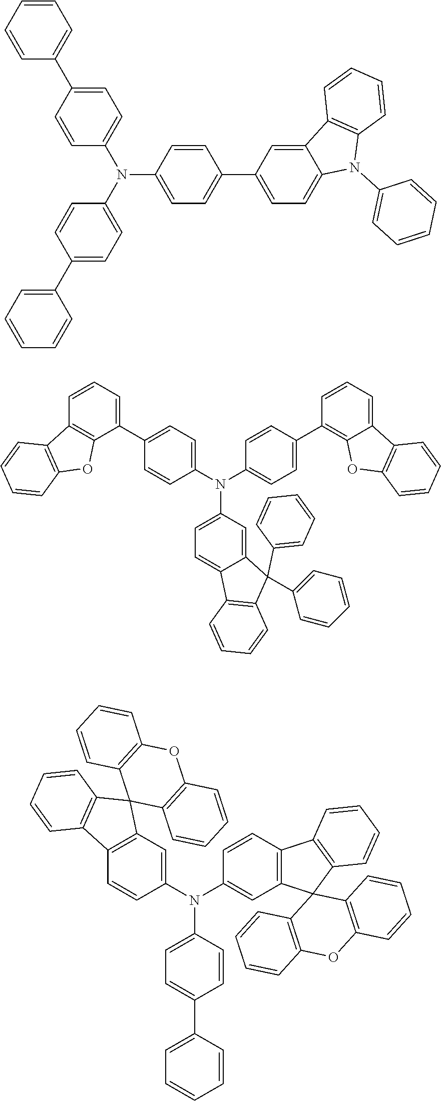 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1]

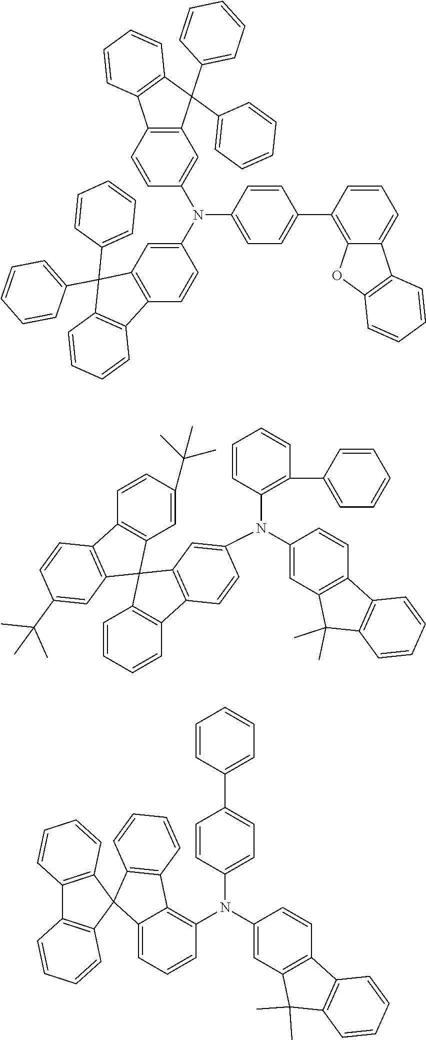 [SMILES: c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; CC1(C)c2ccccc2-c2ccc(N(c3ccc(-c4ccccc4)cc3)c3cccc4c3-c3ccccc3C43c4ccccc4-c4ccccc43)cc21]

### Dopant Material of Light Emitting Layer

The light emitting laver is a laver containing a material having a high light emitting property (a dopant material), and various materials can be used. For example, a fluorescent emitting material or a phosphorescent emitting material can be used as the dopant material. The fluorescent emitting material is a compound that emits light from a singlet excited state, and the phosphorescent emitting material is a compound that emits light from a triplet excited state.

In one embodiment of the organic EL device according to the present invention, the light emitting layer is a single layer.

In another embodiment of the organic EL device according to the present invention, the light emitting layer includes a first light emitting layer and a second light emitting layer.

Examples of a blue-based fluorescent emitting material that can be used for the light emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a green-based fluorescent emitting material that can be used for the light emitting layer include an aromatic amine derivative. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), and N,N,9-triphenylanthracene-9-amine (abbreviation: DPhAPhA).

Examples of a red-based fluorescent emitting material that can be used for the light emitting layer include a tetracene derivative and a diamine derivative. Specific examples thereof include N,N,N′,N′-tetrakis(4-methylphenyl)tetracene-5,11-diamine (abbreviation: p-mPhTD) and 7,14-diphenyl-N,N,N′,N′-tetrakis(4-methylphenyl)acenaphtho[1,2-a]fluoranthene-3,10-diamine (abbreviation: p-mPhAFD).

In one embodiment of the present invention, the light emitting layer preferably contains a fluorescent light emitting material (fluorescent dopant material).

Examples of a blue-based phosphorescent emitting material that can be used for the light emitting layer include a metal complex, such as an iridium complex, an osmium complex, and a platinum complex. Specific examples thereof include bis[2-(4′,6′-difluorophenyl)pyridinato-N,C2′]iridium(III)tetrakis(1-pyrazolyl)borate (abbreviation: FIr6), bis[2-(4′,6′-difluorophenyl)pyridinato-N,C2′]iridium(III)picolinate (abbreviation: FIrpic), bis[2-(3′,5′bistrifluoromethylphenyl)pyridinato-N,C2′]iridium(III)picolinate (abbreviation: Ir(CF3ppy)2(pic)), and bis[2-(4′,6′-difluorophenyl)pyridinato-N,C2′]iridium(III)acetylacetonate (abbreviation: FIracac).

Examples of a green-based phosphorescent emitting material that can be used for the light emitting layer include an iridium complex. Examples thereof include tris(2-phenylpyridinato-N,C2′)iridium(III) (abbreviation: Ir(ppy)3), bis(2-phenylpyridinato-N,C2′)iridium(III)acetylacetonate (abbreviation: Ir(ppy)2(acac)), bis(1,2-diphenyl-1H-benzimidazolato)iridium(III)acetylacetonate (abbreviation: Ir(pbi)2(acac)), and bis(benzo[h]quinolinato)iridium(III)acetylacetonate (abbreviation: Ir(bzq)2(acac)).

Examples of a red-based phosphorescent emitting material that can be used for the light emitting layer include a metal complex, such as an iridium complex, a platinum complex, a terbium complex, and a europium complex. Specific examples thereof include organic metal complexes, such as bis[2-(2′-benzo[4,5-a]thienyl)pyridinato-N,C3′]iridium(III)acetylacetonate (abbreviation: Ir(btp)2(acac)), bis(1-phenylisoquinolinato-N,C2′)iridium(III)acetylacetonate (abbreviation: Ir(piq)2(acac)), (acetylacetonate)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrinplatinum(II) (abbreviation: PtOEP).

Rare earth metal complexes, such as tris(acetylacetonate) (monophenanthroline)terbium(III) (abbreviation: Tb(acac)3(Phen)), tris(1,3-diphenyl-1,3-propanedionate)(monophenanthroline)europium(III) (abbreviation: Eu(DBM)3(Phen)), and tris[l-(2-thenoyl)-3,3,3-trifluoroacetonate](monophenanthroline)europium(III) (abbreviation: Eu(TTA)3(Phen)), emit light from rare earth metal ions (electron transition between different multiplicities), and thus may be used as the phosphorescent emitting material.

### Host Material of Light Emitting Layer

The light emitting layer may have a configuration in which the aforementioned dopant material is dispersed in another material (a host material). The host material is preferably a material that has a higher lowest unoccupied orbital level (LUMO level) and a lower highest occupied orbital level (HOMO level) than the dopant material.

Examples of the host material include:
(1) the inventive compound,
(2) a metal complex, such as an aluminum complex, a beryllium complex, and a zinc complex,
(3) a heterocyclic compound, such as an oxadiazole derivative, a benzimidazole derivative, and a phenanthroline derivative,
(4) a fused aromatic compound, such as a carbazole derivative, an anthracene derivative, a phenanthrene derivative, a pyrene derivative, and a chrysene derivative, or
(5) an aromatic amine compound, such as a triarylamine derivative and a fused polycyclic aromatic amine derivative.

For example,
metal complexes, such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), and bathocuproine (abbreviation: BCP);
fused aromatic compounds, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl(abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and
aromatic amine compounds, such as N,N-diphenyl-9-[4-(10-phenyl-9-anthryl)phenyl]-9H-carbazole-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N,9-diphenyl-N-{4-[4-(10-phenyl-9-anthryl)phenyl]phenyl}-9H-carbazole-3-amine (abbreviation: PCAPBA), N-(9,10-diphenyl-2-anthryl)-N,9-diphenyl-9H-carbazole-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB) can be used. A plurality of host materials may be used.

In particular, in the case of a blue fluorescent device, it is preferred to use the following anthracene compounds as the host material.

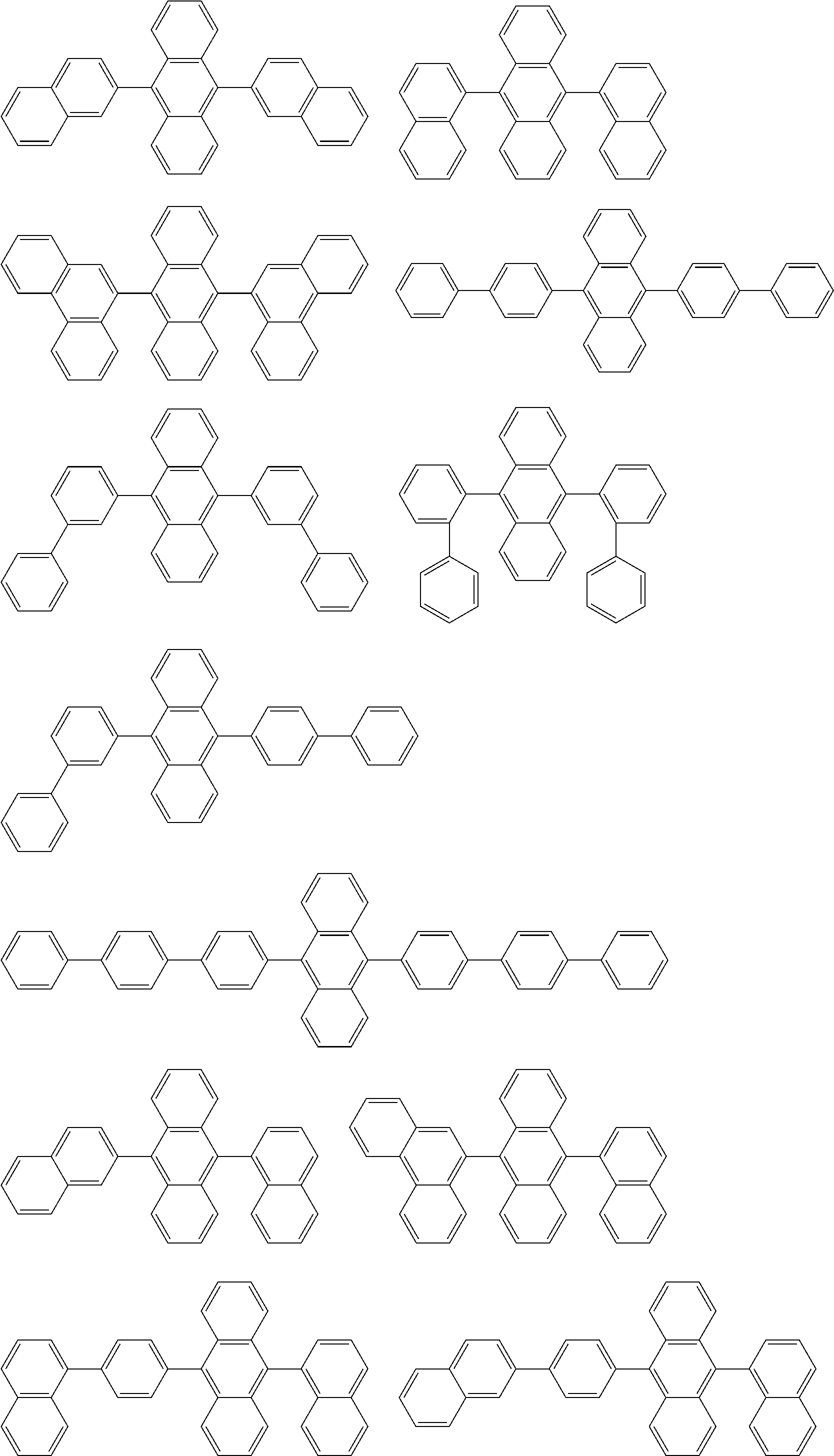

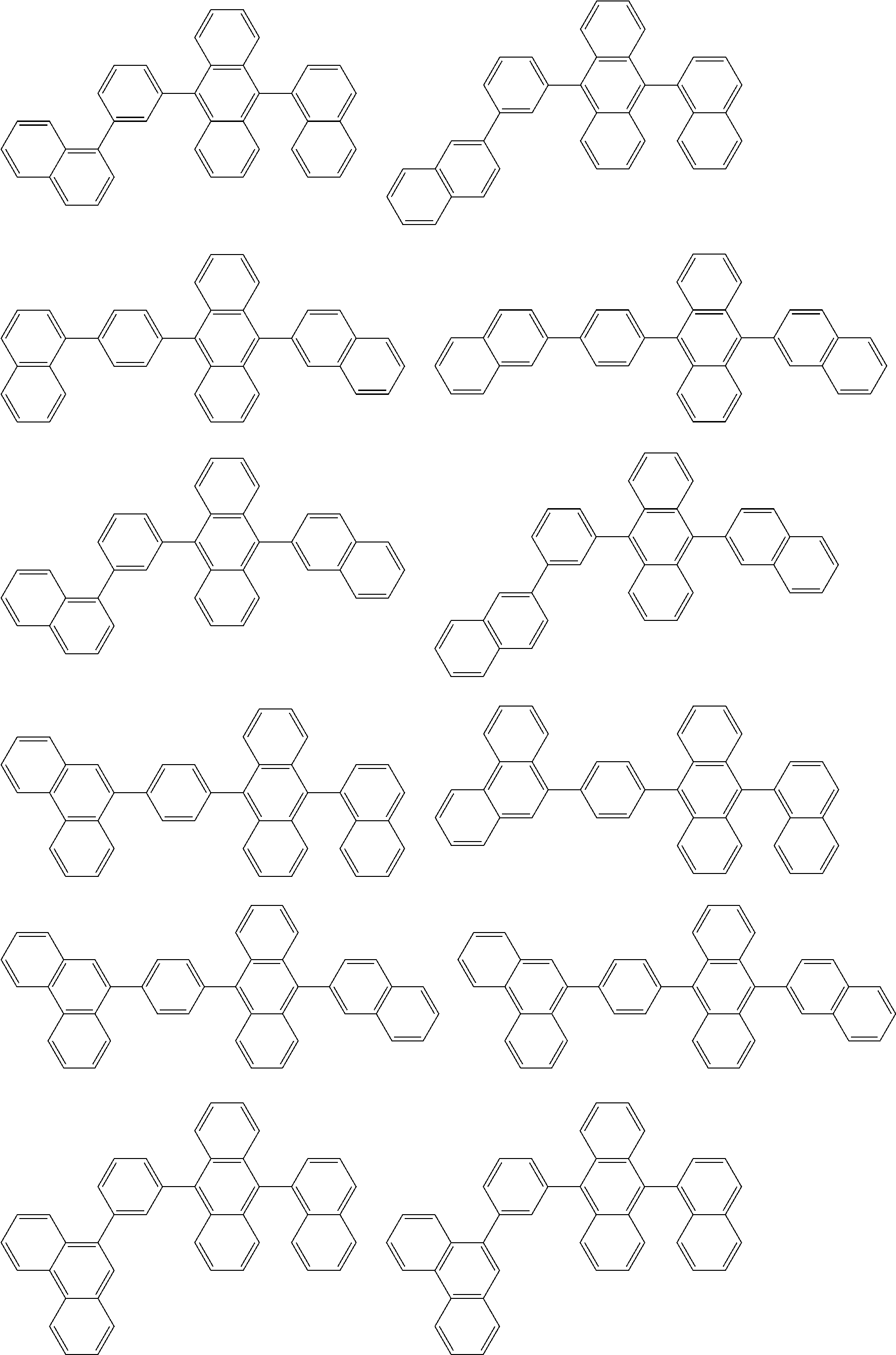

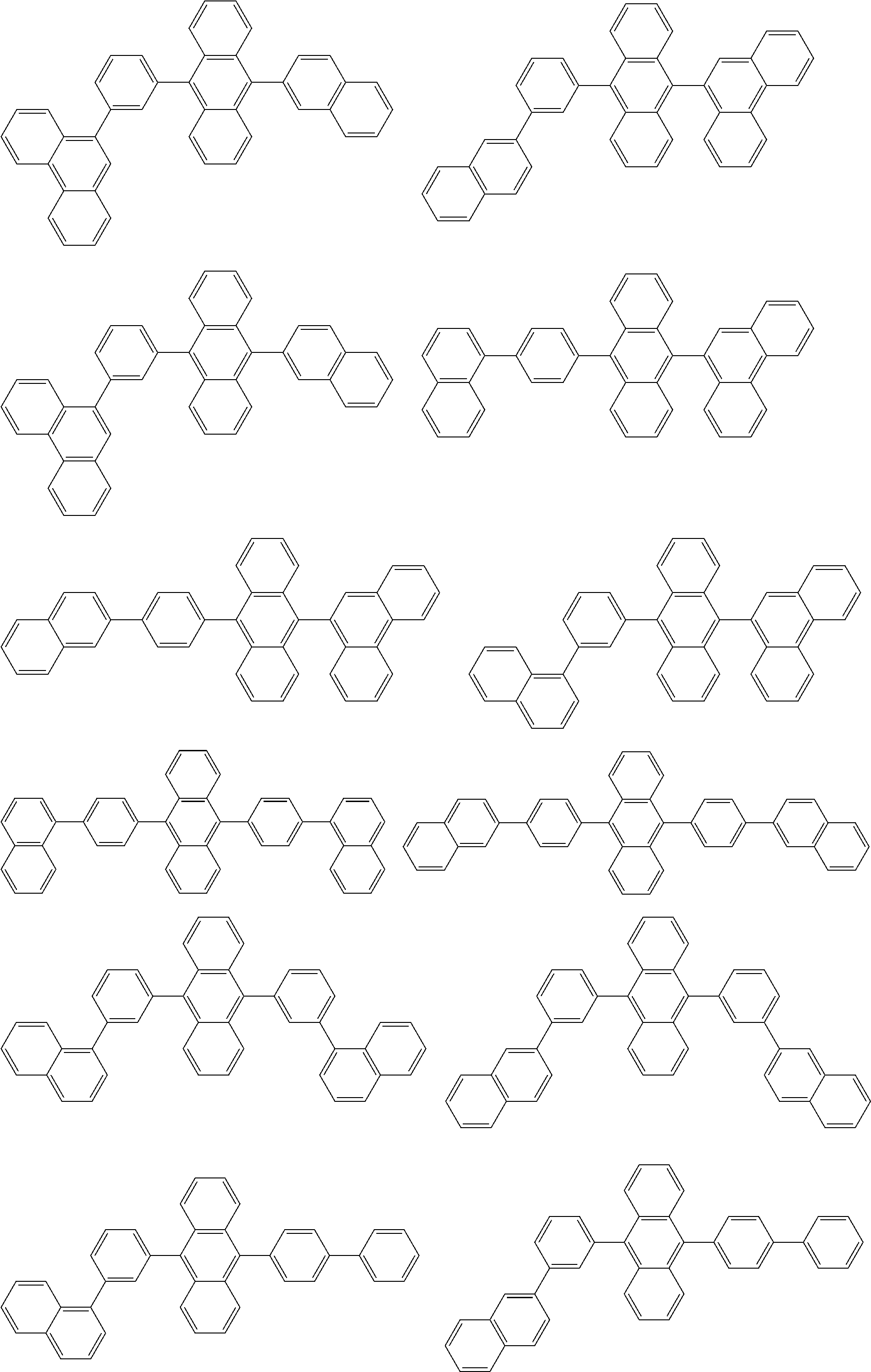

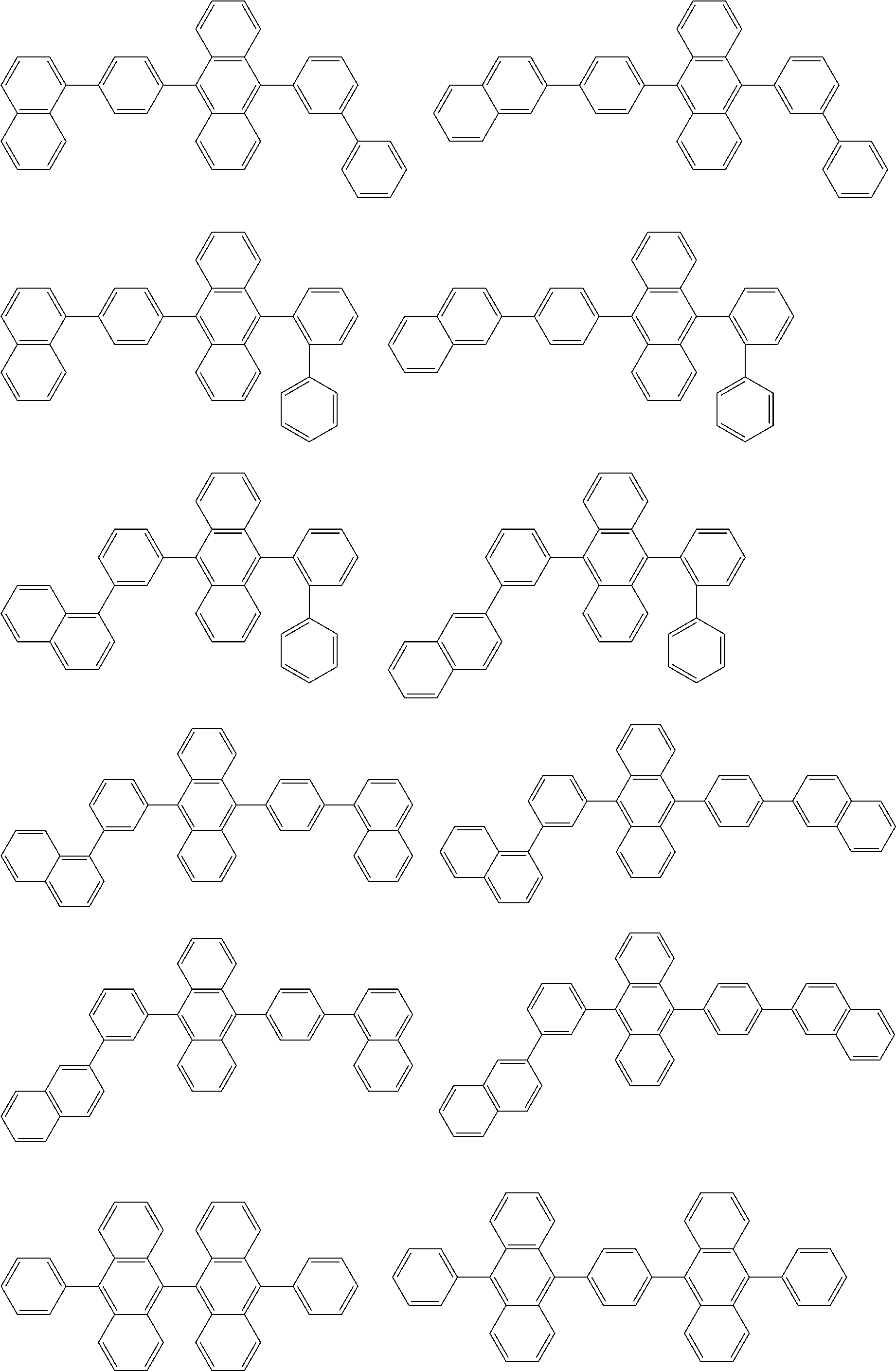

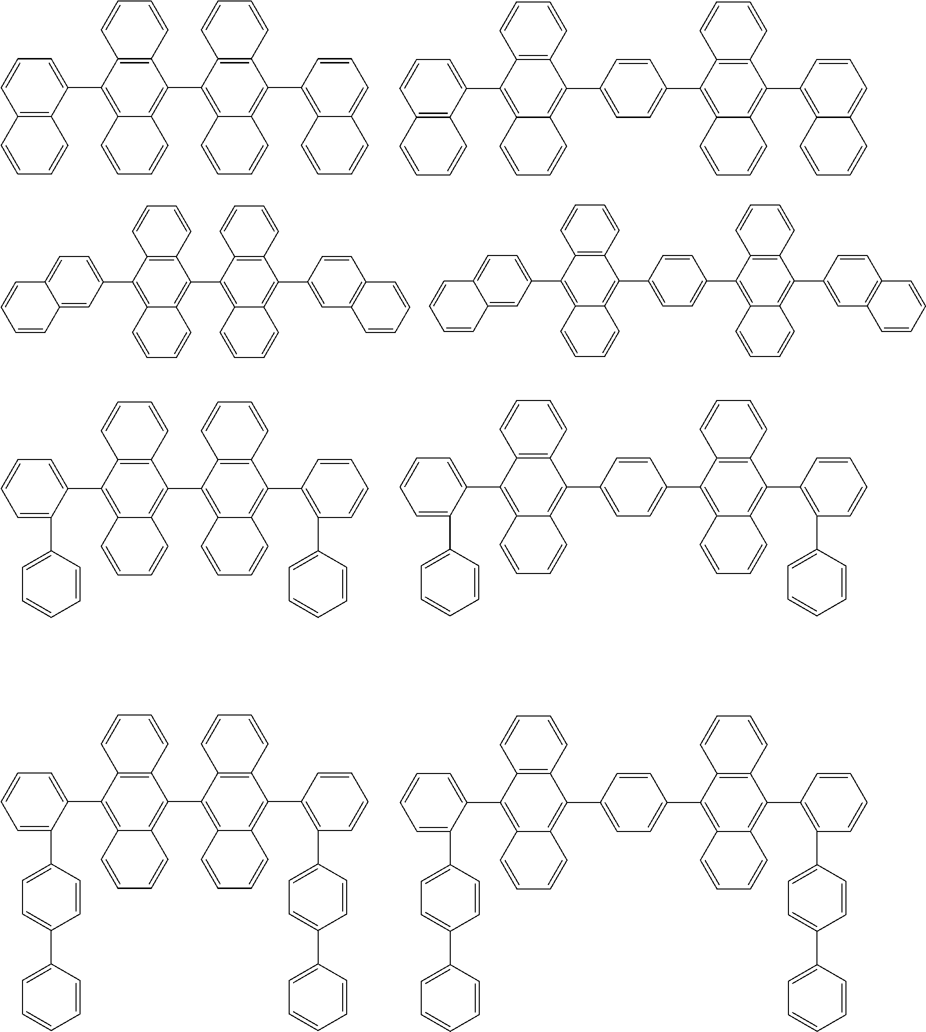

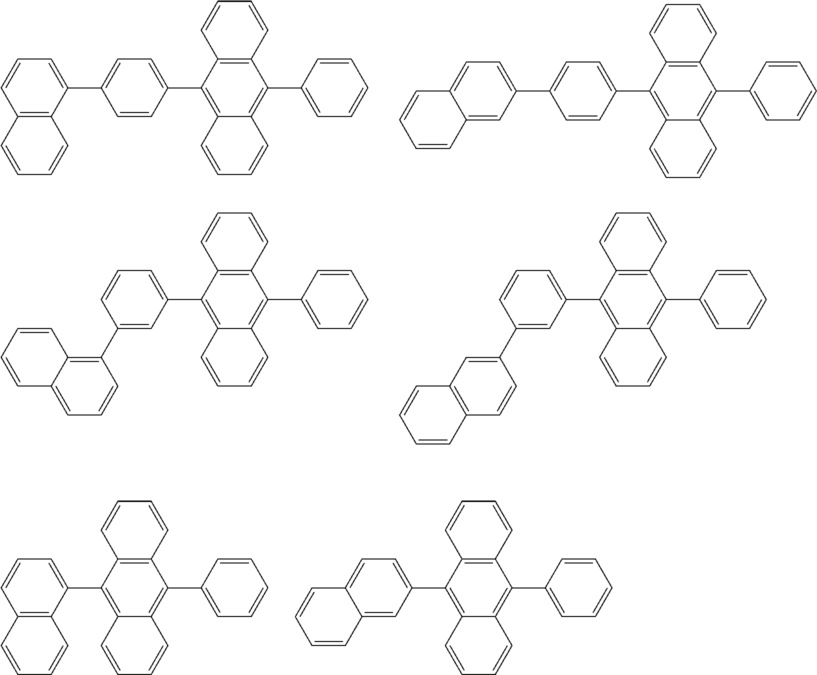

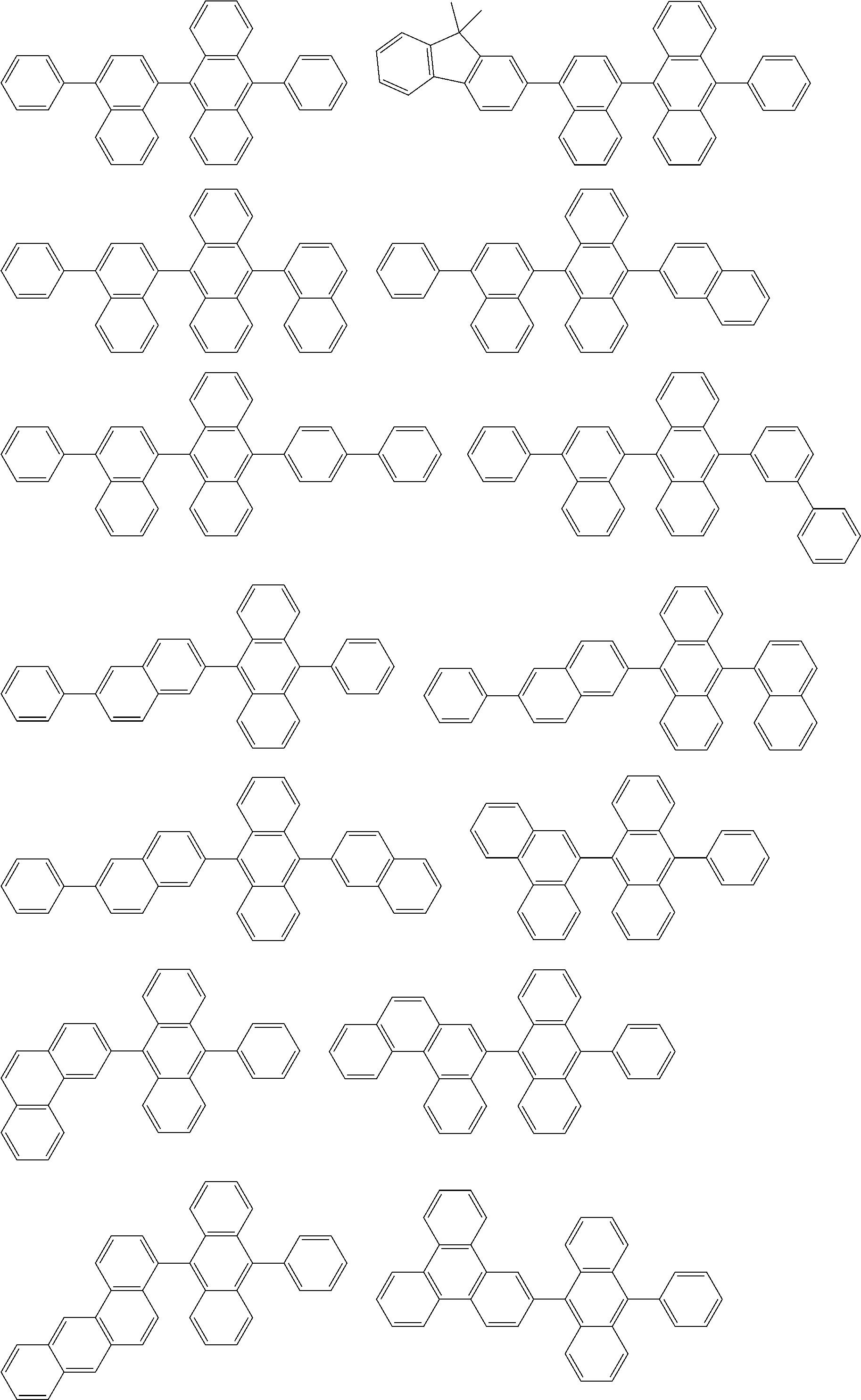

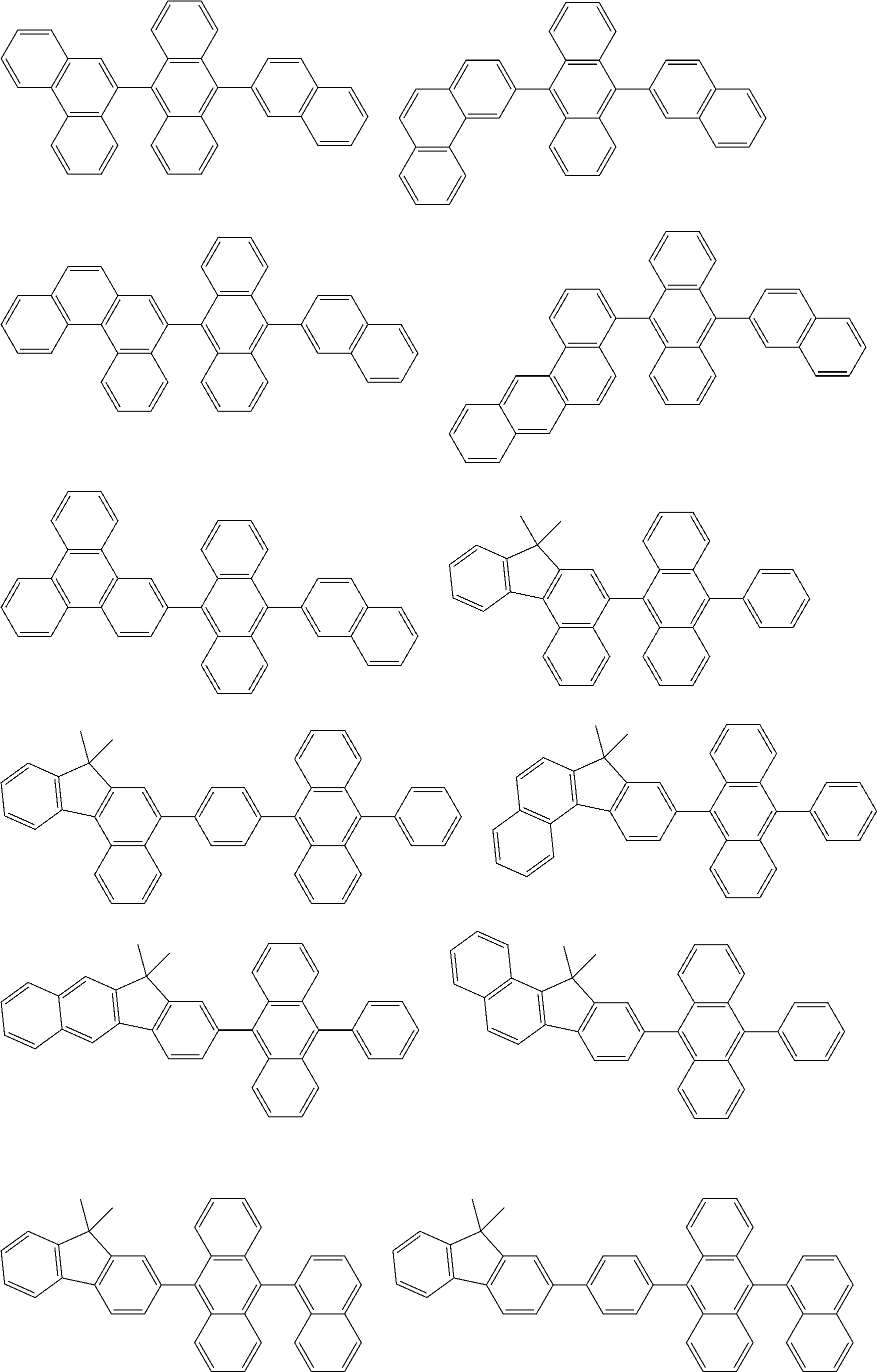

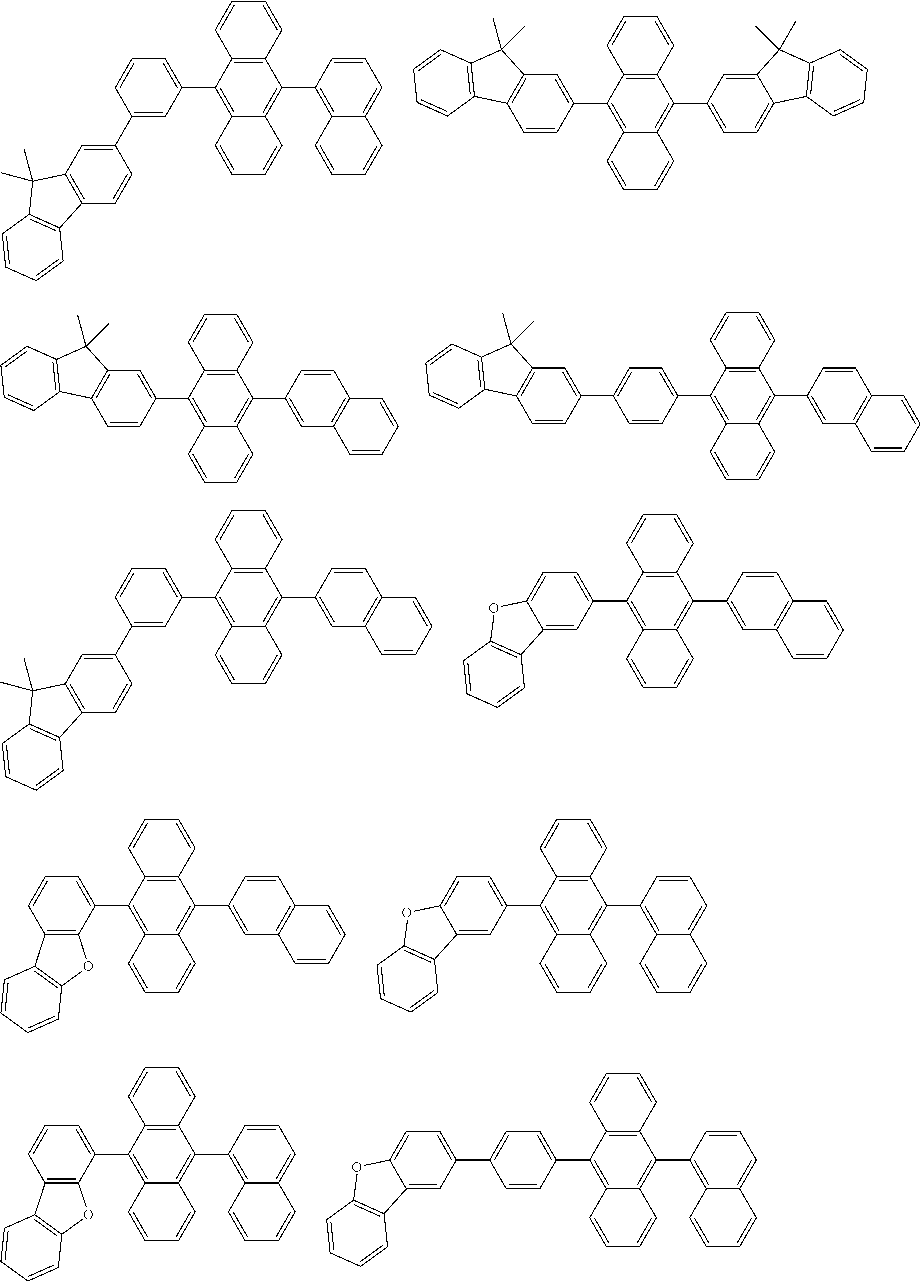

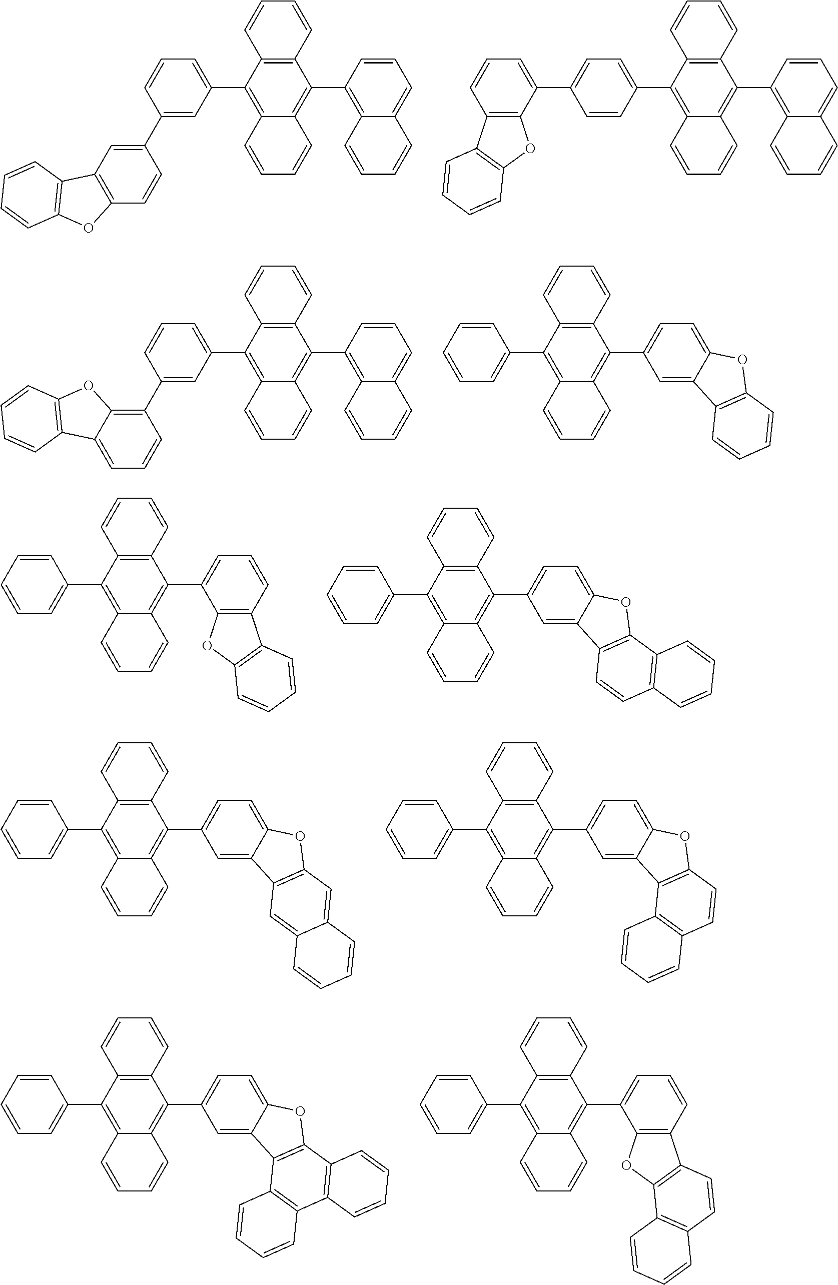

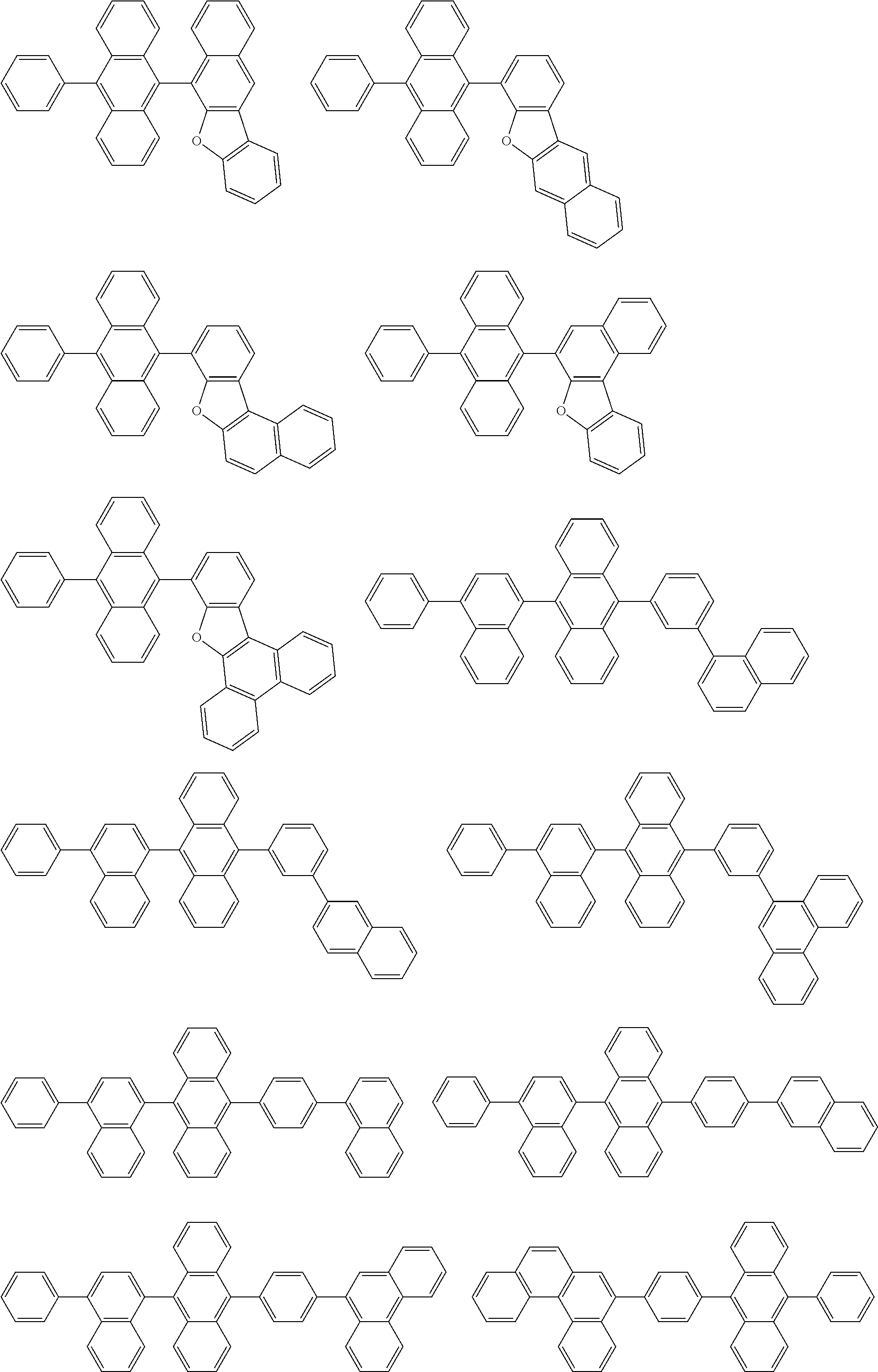

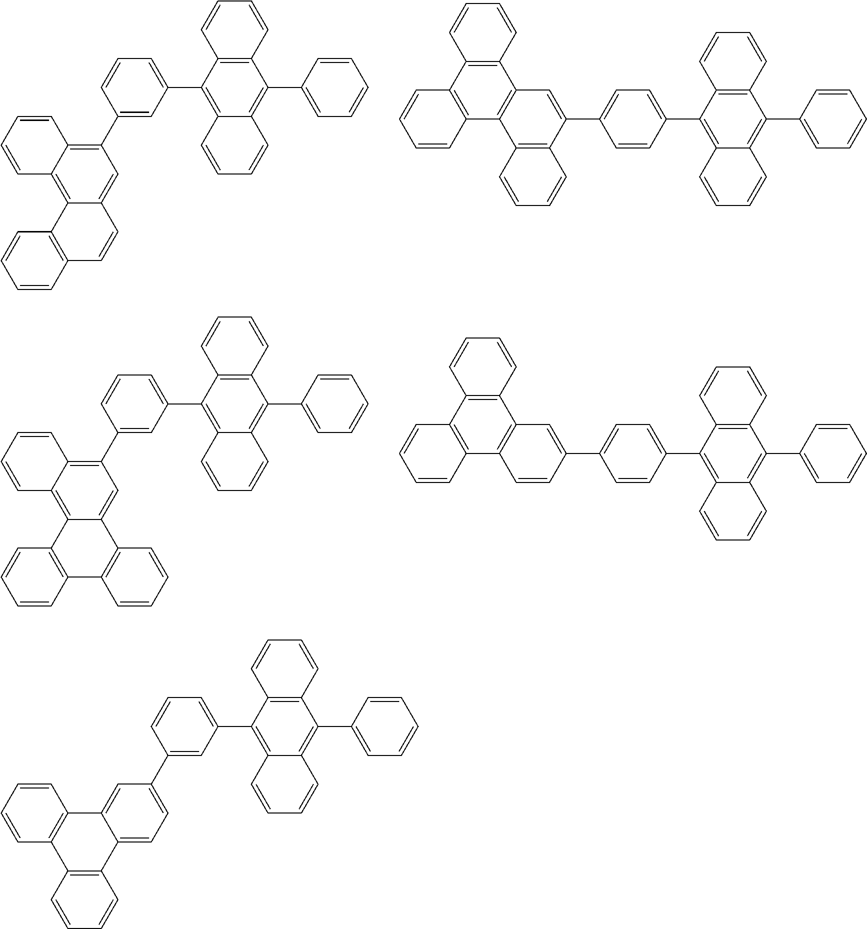

In one embodiment of the organic EL device according to the present invention, when the light emitting layer includes a first light emitting layer and a second light emitting layer, at least one of components constituting the first light emitting layer is different from a component constituting the second light emitting layer. For example, a dopant material contained in the first light emitting layer may be different from a dopant material contained in the second light emitting layer, or a host material contained in the first light emitting layer may be different from a host material contained in the second light emitting layer.

In the organic EL device according to the present embodiment, the light emitting layer may contain a light emitting compound exhibiting fluorescence emission having a main peak wavelength of 500 nm or less.

The method for measuring the main peak wavelength of the compound is as follows. A 5 μmol/L toluene solution of a compound to be measured is prepared and placed in a quartz cell, and the emission spectrum (vertical axis: emission intensity, horizontal axis: wavelength) of the sample is measured at room temperature (300K). The emission spectrum can be measured using a fluorescence spectrophotometer (device name: F-7000) manufactured by Hitachi High-Tech Science Corporation. Note that the emission spectrum measuring device is not limited to the device used here.

In the emission spectrum, the peak wavelength of the emission spectrum at which the emission intensity becomes maximum is defined as a main peak wavelength. In the description herein, the main peak wavelength is sometimes referred to as a fluorescence emission main peak wavelength (FL-peak).

The light emitting compound exhibiting fluorescence emission having a main peak wavelength of 500 nm or less may be the dopant material or the host material.

In the case where the light emitting layer is a single layer, only one of the dopant material and the host material may be a light emitting compound exhibiting fluorescence emission having a main peak wavelength of 500 nm or less, or both of the materials may be a light emitting compound exhibiting fluorescence emission having a main peak wavelength of 500 nm or less.

In the case where the light emitting layer includes a first light emitting layer and a second light emitting layer, only one of the first light emitting layer and the second light emitting layer may contain a light emitting compound exhibiting fluorescence emission having a main peak wavelength of 500 nm or less, or both of the light emitting layers may contain a light emitting compound exhibiting fluorescence emission having a main peak wavelength of 500 nm or less. In the case where the first light emitting layer contains a light emitting compound exhibiting fluorescence emission having a main peak wavelength of 500 nm or less, only one of the dopant material and the host material contained in the first light emitting layer may be a light emitting compound exhibiting fluorescence emission having a main peak wavelength of 500 nm or less, or both of the materials may be a light emitting compound exhibiting fluorescence emission having a main peak wavelength of 500 nm or less. In addition, in the case where the second light emitting layer contains a light emitting compound exhibiting fluorescence emission having a main peak wavelength of 500 nm or less, only one of the dopant material and the host material contained in the second light emitting layer may be a light emitting compound exhibiting fluorescence emission having a main peak wavelength of 500 nm or less, or both of the materials may be a light emitting compound exhibiting fluorescence emission having a main peak wavelength of 500 nm or less.

### (Electron Transporting Layer)

The electron transporting layer is a layer containing a material having a high electron transporting capability (an electron transporting material) and is provided between the light emitting layer and the cathode, or between the electron injecting layer, if exists, and the light emitting layer.

The electron transporting layer may have a single layer structure or a multilayer structure including two or more layers. For example, the electron transporting layer may be a two layer structure including a first electron transporting layer (on the anode side) and a second electron transporting layer (on the cathode side). In one embodiment of the present invention, the electron transporting layer having a single layer structure is preferably adjacent to the light emitting layer, and the electron transporting layer closest to the anode in the multilayer structure, for example, the first electron transporting layer having a two layer structure is preferably adjacent to the light emitting layer. In another embodiment of the present invention, a hole blocking layer to be described later may be interposed between the electron transporting layer having a single layer structure and the light emitting layer or between the electron transporting layer closest to the light emitting layer in the multilayer structure and the light emitting layer.

As the electron transporting layer, for example,
(1) a metal complex, such as an aluminum complex, a beryllium complex, and a zinc complex;
(2) a heteroaromatic compound, such as an imidazole derivative, a benzimidazole derivative, an azine derivative, a carbazole derivative, and a phenanthroline derivative; and
(3) a high-molecular weight compound can be used.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzxazol-2-yl)stilbene (abbreviation: BzOs).

Examples of the high-molecular weight compound include poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy).

The above-mentioned materials are materials having an electron mobility of 10⁻⁶ cm²/Vs or more. Materials other than those as mentioned above may also be used in the electron transporting layer so long as they are materials high in the electron transporting capability rather than in the hole transporting capability.

### (Electron Injecting Layer)

The electron injecting layer is a layer containing a material having a high electron injection capability. As the electron injecting layer, alkali metals, such as lithium (Li) and cesium (Cs), alkaline earth metals, such as magnesium (Mg), calcium (Ca), and strontium (Sr), rare earth metals, such as europium (Eu) and ytterbium (Yb), and compounds containing these metals can be used. Examples of the compounds include an alkali metal oxide, an alkali metal halide, an alkali metal-containing organic complex, an alkaline earth metal oxide, an alkaline earth metal halide, an alkaline earth metal-containing organic complex, a rare earth metal oxide, a rare earth metal halide, and a rare earth metal-containing organic complex. These compounds may be used as a mixture of a plurality thereof.

In addition, a material having an electron transporting capability, in which an alkali metal, an alkaline earth metal, or a compound thereof is contained, specifically Alq in which magnesium (Mg) is contained may be used. In this case, electron injection from the cathode can be more efficiently performed.

Otherwise, in the electron injecting layer, a composite material obtained by mixing an organic compound with an electron donor may be used. Such a composite material is excellent in the electron injection capability and the electron transporting capability because the organic compound receives electrons from the electron donor. In this case, the organic compound is preferably a material excellent in transporting received electrons, and specifically, examples thereof include a material constituting the aforementioned electron transporting layer (such as a metal complex and a heteroaromatic compound). As the electron donor, a material having an electron donation property for the organic compound may be used. Specifically, alkali metals, alkaline earth metals, and rare earth metals are preferred, and examples thereof include lithium, cesium, magnesium, calcium, erbium, and ytterbium. In addition, an alkali metal oxide or an alkaline earth metal oxide is preferred, and examples thereof include lithium oxide, calcium oxide, and barium oxide. In addition, a Lewis base, such as magnesium oxide, can also be used. In addition, an organic compound, such as tetrathiafulvalene (abbreviation: TTF), can also be used.

### (Cathode)

It is preferred that a metal, an alloy, an electrically conductive compound, or a mixture thereof which has a low work function (specifically 3.8 eV or less) is used for the cathode. Specific examples of such a cathode material include elements belonging to Group 1 or Group 2 of the periodic table of the elements, that is, alkali metals, such as lithium (Li) and cesium (Cs), alkaline earth metals, such as magnesium (Mg), calcium (Ca), and strontium (Sr), and alloys containing these (such as MgAg, and AlLi), and rare earth metals, such as europium (Eu), and ytterbium (Yb) and alloys containing these.

When the cathode is formed by using the alkali metals, the alkaline earth metals, and the alloys containing these, a vacuum vapor deposition method or a sputtering method can be adopted. In addition, when a silver paste is used, a coating method or an inkjet method can be adopted.

By providing the electron injecting layer, the cathode can be formed using various conductive materials, such as A1, Ag, ITO, graphene, and indium oxide-tin oxide containing silicon or silicon oxide regardless of the magnitude of a work function. Such a conductive material can be deposited by using a sputtering method, an inkjet method, or a spin coating method.

### (Insulating Layer)

The organic EL device applies an electric field to an ultrathin film, and thus, pixel defects are likely to occur due to leaks or short-circuiting. In order to prevent this, an insulating layer formed of an insulating thin film layer may be inserted between a pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate of these may also be used.

### (Space Layer)

The space layer is, for example, a layer provided between a fluorescent light emitting layer and a phosphorescent light emitting layer for the purpose of preventing excitons generated in the phosphorescent light emitting layer from diffusing into the fluorescent light emitting layer, or adjusting a carrier balance, in the case where the fluorescent light emitting layers and the phosphorescent light emitting layers are stacked. The space layer can also be provided among the plurality of phosphorescent light emitting layers.

Since the space layer is provided between the light emitting layers, it is preferably formed using a material having both an electron transporting capability and a hole transporting capability. Also, one having a triplet energy of 2.6 eV or more is preferred in order to prevent triplet energy diffusion in the adjacent phosphorescent light emitting layer. Examples of the material used for the space layer are the same as those used for the hole transporting layer described above.

### (Blocking Layer)

The blocking layer such as the electron blocking layer, the hole blocking layer, or the exciton blocking layer may be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function of preventing excitons generated in the light emitting layer from diffusing into the surrounding layers, and trapping the excitons within the light emitting layer.

Each layer of the organic EL device may be formed by a conventionally known vapor deposition method or coating method. For example, formation can be performed by a known method using a vapor deposition method such as a vacuum vapor deposition method, or a molecular beam vapor deposition method (MBE method), or a coating method using a solution of a compound for forming a layer, such as a dipping method, a spin-coating method, a casting method, a bar-coating method, and a roll-coating method.

The film thickness of each layer is not particularly limited, but is typically 5 nm to 10 m, and more preferably 10 nm to 0.2 m because in general, when the film thickness is too small, defects such as pinholes are likely to occur, and conversely, when the film thickness is too large, a high driving voltage is required and the efficiency decreases.

In one embodiment of the organic EL device of the present invention, the sum of the thicknesses of the first hole transporting layer and the second hole transporting layer is 30 nm or more and 150 nm or less. In this case, it is preferably 40 nm or more and 130 nm or less.

In one embodiment of the organic EL device of the present invention, the thickness of the second hole transporting layer is 20 nm or more. The thickness of the second hole transporting layer is preferably 25 nm or more, and more preferably 35 nm or more, and is preferably 100 nm or less.

Further, in one embodiment of the organic EL device of the present invention, the thickness of the hole transporting layer adjacent to the light emitting layer is 20 nm layer or more. The thickness of the hole transporting layer adjacent to the light emitting layer is preferably 25 nm or more, and more preferably 30 nm or more, and is preferably 100 nm or less.

In one embodiment of the organic EL device of the present invention, a film thickness D1 of the first hole transporting layer and a film thickness D2 of the second hole transporting layer satisfy the relationship of 0.3<D2/D1<4.0. Preferably, the film thickness D1 of the first hole transporting layer and the film thickness D2 of the second hole transporting layer satisfy the relationship of 0.5<D2/D1<3.5. More preferably, the film thickness D1 of the first hole transporting layer and the film thickness D2 of the second hole transporting layer satisfy the relationship of 0.75<D2/D1<3.0.

Embodiments of the organic EL device of the present invention include, for example, the following:
in the organic EL device having the hole transporting layer of a two layer structure,
a first embodiment in which the second hole transport layer contains at least one of the compound of the present invention and the compound A and the first hole transporting layer does not contain the compound of the present invention and the compound A;
a second embodiment in which both the first hole transporting layer and the second hole transporting layer contain at least one of the compound of the present invention and the compound A; and
a third embodiment in which the first hole transporting layer contains at least one of the compound of the present invention and the compound A and the second hole transporting layer does not contain the compound of the present invention and the compound A; and
in the organic EL device having the hole transporting layer of a three layer structure,
a fourth embodiment in which the first hole transporting layer contains at least one of the compound of the present invention and the compound A and the second and third hole transporting layers do not contain the compound of the present invention and the compound A;
a fifth embodiment in which the second hole transport layer contains at least one of the compound of the present invention and the compound A and the first and third hole transporting layers do not contain the compound of the present invention and the compound A;
a sixth embodiment in which the third hole transport layer contains at least one of the compound of the present invention and the compound A and the first and second hole transporting layers do not contain the compound of the present invention and the compound A;
a seventh embodiment in which the first and second hole transporting layers contain at least one of the compound of the present invention and the compound A and the third hole transporting layer does not contain the compound of the present invention and the compound A;
an eighth embodiment in which the first and third hole transporting layers contain at least one of the compound of the present invention and the compound A and the second hole transporting layer does not contain the compound of the present invention and the compound A;
a tenth embodiment in which the second and third hole transport layers contain at least one of the compound of the present invention and the compound A and the first hole transporting layer does not contain the compound of the present invention and the compound A; and
a tenth embodiment in which all of the first to third hole transporting layers contain at least one of the compound of the present invention and the compound A.

### [Electronic Device]

The organic EL device can be used for electronic devices, such as display components of an organic EL panel module, display devices of a television, a mobile phone and a personal computer, and light emitting devices of lightings and vehicular lamps.

### EXAMPLES

Hereinafter, the present invention will be described in more detail with reference to Examples, but the present invention is not limited thereto.

Compounds used for Production of Organic EL Devices of Examples 1 to 5

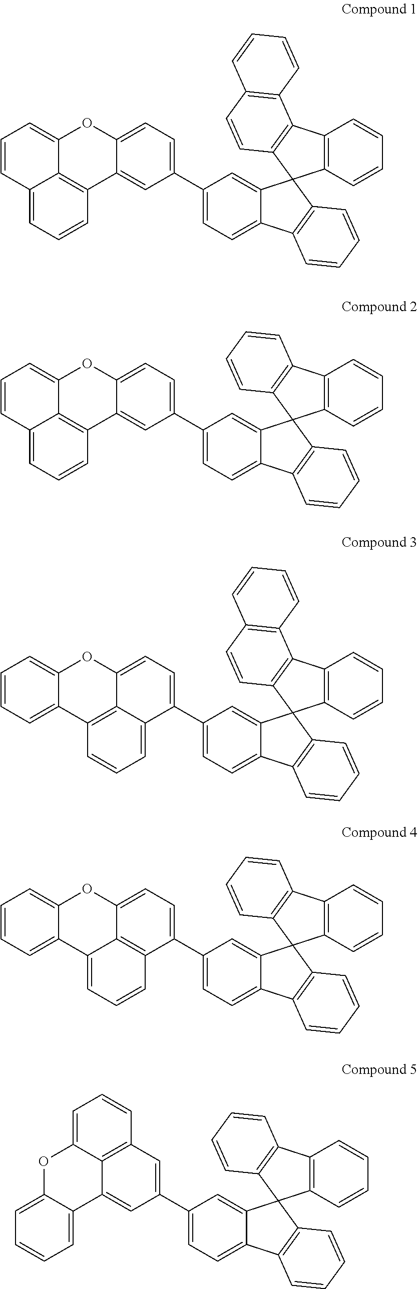 [SMILES: c1ccc2c(c1)-c1ccc(-c3ccc4c(c3)-c3cccc5cccc(c35)O4)cc1C21c2ccccc2-c2c1ccc1ccccc21 ; c1ccc2c(c1)-c1ccccc1C21c2ccccc2-c2ccc(-c3ccc4c(c3)-c3cccc5cccc(c35)O4)cc21 ; c1ccc2c(c1)Oc1ccc(-c3ccc4c(c3)C3(c5ccccc5-4)c4ccccc4-c4c3ccc3ccccc43)c3cccc-2c13 ; c1ccc2c(c1)Oc1ccc(-c3ccc4c(c3)C3(c5ccccc5-c5ccccc53)c3ccccc3-4)c3cccc-2c13 ; c1ccc2c(c1)Oc1cccc3cc(-c4ccc5c(c4)C4(c6ccccc6-c6ccccc64)c4ccccc4-5)cc-2c13]

Comparative Compounds used for Production of Organic EL Devices of Comparative Examples 1 to 3

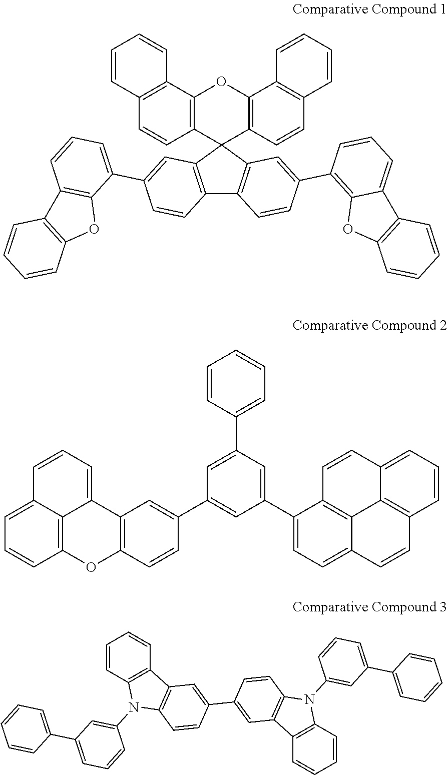 [SMILES: c1ccc2c3c(ccc2c1)C1(c2cc(-c4cccc5c4oc4ccccc45)ccc2-c2ccc(-c4cccc5c4oc4ccccc45)cc21)c1ccc2ccccc2c1O3 ; c1ccc(-c2cc(-c3ccc4c(c3)-c3cccc5cccc(c35)O4)cc(-c3ccc4ccc5cccc6ccc3c4c56)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

Comparative Compound 1
Comparative Compound 2
Comparative Compound 3

Other Compounds used for Production of Organic EL Devices of Examples 1 to 5 and Comparative Examples 1 to 3

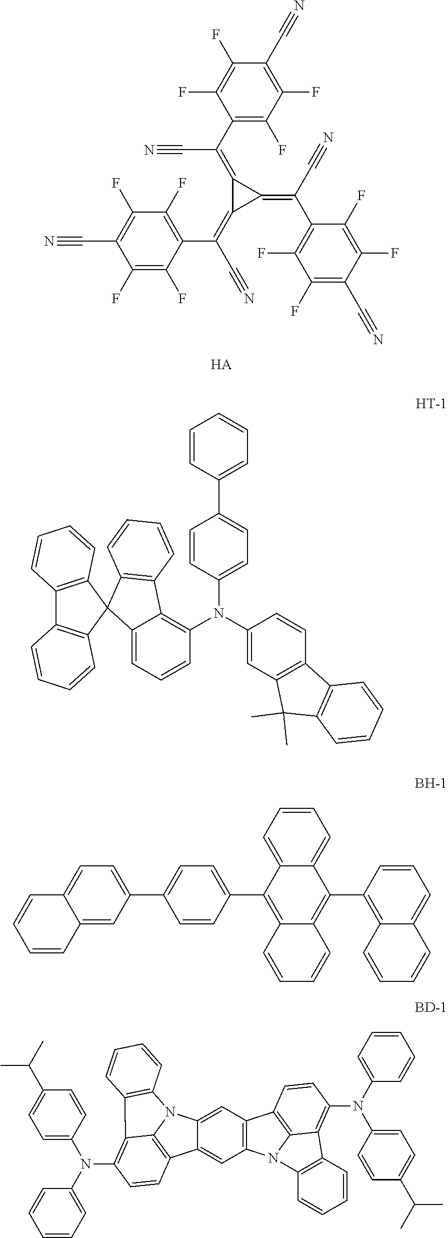

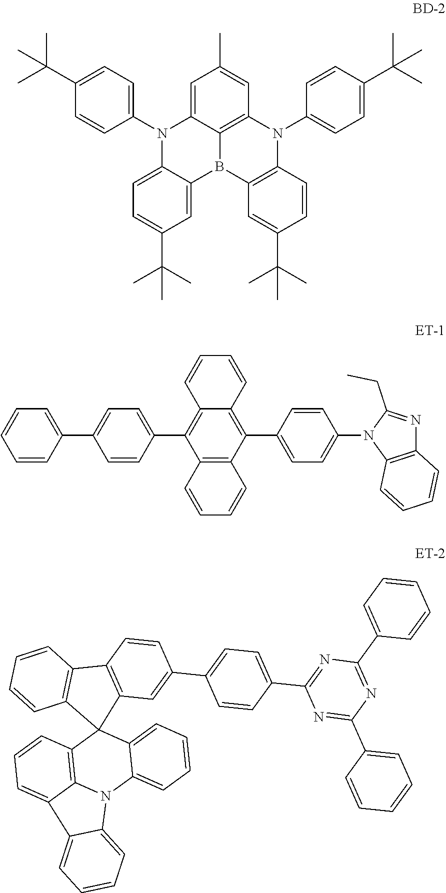 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; CCc1nc2ccccc2n1-c1ccc(-c2c3ccccc3c(-c3ccc(-c4ccccc4)cc3)c3ccccc23)cc1 ; c1ccc(-c2nc(-c3ccccc3)nc(-c3ccc(-c4ccc5c(c4)C4(c6ccccc6-5)c5ccccc5-n5c6ccccc6c6cccc4c65)cc3)n2)cc1]

### Production of Organic EL Devices 1

### Example 1

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the transparent electrode was mounted on a substrate holder of a vacuum vapor deposition apparatus, and firstly, Compound HT-1 and Compound HA were vapor co-deposited on the surface having the transparent electrode formed thereon, so as to cover the transparent electrode, resulting in a hole injecting layer with a film thickness of 10 nm. The mass ratio of Compound HT-1 to Compound HA (HT-1:HA) was 85:15.

Subsequently, on this hole injecting layer, Compound HT-1 was vapor deposited to form a first hole transporting layer with a film thickness of 80 nm.

Subsequently, Compound 1 as Compound HT-2 (second hole transporting material) was vapor deposited on the first hole transporting layer to form a second hole transporting layer with a film thickness of 10 nm.

Subsequently, Compound BH-1 (host material) and Compound BD-1 as the dopant material BD were vapor co-deposited on the second hole transporting layer to form a light emitting layer with a film thickness of 25 nm. The mass ratio of Compound BH-1 to Compound BD-1 (BH-1:BD-1) was 98:2.

Subsequently, on this light emitting layer, Compound ET-1 and Compound ET-2 were vapor co-deposited to form an electron transporting layer with a film thickness of 20 nm. The mass ratio of Compound ET-1 to Compound ET-2 (ET-1:ET-2) was 50:50.

Next, LiF was vapor deposited on this electron transporting layer to form an electron injecting electrode having a film thickness of 1 nm.

Next, metal A1 was vapor deposited on the electron injecting electrode to form a metal cathode having a film thickness of 50 nm.

The layer configuration of the organic EL device of Example 1 thus obtained is shown below.

$$ITO ( 130 ) / HT - 1 : HA = 85 : 15 ( 10 ) / HT - 1 ( 80 ) / HT - 2 ( 10 ) / BH - 1 : BD = 98 : 2 ( 25 ) / ET - 1 : ET - 2 = 50 : 50 ( 20 ) / LiF ( 1 ) / Al ( 50 )$$

In the layer configuration, the numeral in parentheses indicates the film thickness (nm), and the ratio is a mass ratio.

### Examples 2 to 4

Organic EL devices were produced in the same manner as in Example 1 except that the second hole transporting layer material HT-2 was changed from Compound 1 to Compounds 2, 3, and 4, respectively, as shown in Table 1 below.

### Example 5

An organic EL device was produced in the same manner as in Example 1 except that the second hole transporting layer material HT-2 was changed from Compound 1 to Compound 5 as shown in Table 1 below, and the dopant material BD was changed from Compound BD-1 to Compound BD-2.

### Comparative Examples 1 to 3

Organic EL devices were produced in the same manner as in Example 1 except that the second hole transporting layer material HT-2 was changed from Compound 1 to Comparative Compounds 1, 2, and 3, respectively, as shown in Table 1 below.

### Evaluation of Organic EL Devices 1

For the organic EL devices produced in Examples 1 to 5 and Comparative Examples 1 to 3, a voltage was applied to the organic EL device so that the current density was 10 mA/cm², and the external quantum efficiency (EQE) was evaluated.

In addition, for each of the organic EL devices described above, a voltage was applied to the organic EL device so that the current density was 50 mA/cm², and the 95% life (LT95) was evaluated. Here, the 95% life (LT95) refers to a time (hr) until the brightness decreased to 95% of the initial brightness in the constant-current driving.

The results are shown in Table 1.

TABLE 1

| | Second hole | Dopant | EQE | LT95 [h] |
|---|---|---|---|---|
| | transporting | material | @10 | @50 |
| | material (HT-2) | (BD) | mA/cm² | mA/cm² |
| Example 1 | Compound 1 | BD-1 | 10.5 | 58 |
| Example 2 | Compound 2 | BD-1 | 10.0 | 66 |
| Example 3 | Compound 3 | BD-1 | 10.2 | 62 |
| Example 4 | Compound 4 | BD-1 | 10.4 | 75 |
| Example 5 | Compound 5 | BD-2 | 9.6 | 73 |
| Comparative | Comparative | BD-1 | 9.4 | 52 |
| Example 1 | Compound 1 | | | |
| Comparative | Comparative | BD-1 | 9.3 | 61 |
| Example 2 | Compound 2 | | | |
| Comparative | Comparative | BD-1 | 9.4 | 34 |
| Example 3 | Compound 3 | | | |

As is clear from the results of Table 1, it is found that the compounds satisfying the requirements of the present invention and included in the above formula (1) (Compounds 1 to 5 of Examples 1 to 5) exhibit remarkably improved values of EQE and LT95 as compared with the monoamines not satisfying the requirements of the present invention and not included in the above formula (1) (Comparative Compounds 1 to 3 of Comparative Examples 1 to 3).

### <Measurement of Physical Properties of Compound 1 and Comparative Compound 3>

### (Highest Occupied Molecular Orbital Energy Level HOMO)

The ionization potential and the highest occupied molecular orbital energy level HOMO of Compound 1 and Comparative Compound 3 were measured using a photoelectron spectrometer (“AC-3” manufactured by RIKEN KEIKI CO., LTD) in the atmosphere. Specifically, the ionization potential and the highest occupied molecular orbital energy level HOMO of Compound 1 and Comparative Compound 3 were measured by irradiating a material, on which a film was formed to a film thickness of 50 nm, with light and measuring the amount of electrons generated by charge separation at that time. The ionization potential may be referred to as Ip. In addition, the value of the highest occupied molecular orbital energy level HOMO corresponds to a value obtained by adding a negative sign to the value of the ionization potential. For example, since the value of the ionization potential of Compound 1 shown in Table 2 is 5.75 eV, the value of the highest occupied molecular orbital energy level HOMO of Compound 1 is −5.75 eV

### (Electron Affinity Af and Lowest Unoccupied Molecular Orbital Energy Level LUMO)

The electron affinity Af was calculated by the following mathematical expression (Equation 5).

$$Af = - 1.19 \times ( Ere - Efc ) - 4.78 eV \qquad ( Equation 5 )$$

Here, each symbol means the following.
Ere: first reduction potential (DPV, negative scan)
Efc: first oxidation potential of ferrocene (DPV, positive scan), (ca. +0.55 V vs Ag/AgCl)

The oxidation reduction potential was measured by a differential pulse voltammetry (DPV) method based on the following reference documents using an electrochemical analyzer (manufactured by ALS company: CHI630B).

N,N-dimethylformamide (DMF) was used as a solvent, and the sample concentration was 1.0 mmol/L. Tetrabutylammonium hexafluorophosphate (TBHP) (100 mmol/L) was used as a supporting electrolyte. Glassy carbon and Pt were used as the working electrode and the counter electrode, respectively.
(References) M. E. Thompson, et. al., Organic Electronics, 6 (2005), p. 11-20, Organic Electronics, 10 (2009), p. 515-520

A value obtained by adding a negative sign to the value of the electron affinity Af was defined as the lowest unoccupied molecular orbital energy level LUMO.

### (Triplet Energy T₁)

The triplet energy T₁ was measured by the following method.

A compound to be measured was dissolved in EPA (diethyl ether:isopentane:ethanol=5:5:2 (volume ratio)) at a concentration of 10⁻⁵ mol/L or more and 10⁻⁴ mol/L or less to prepare a solution, and the solution was placed in a quartz cell to be used as a measurement sample. For this measurement sample, a phosphorescence spectrum (vertical axis: phosphorescence emission intensity, horizontal axis: wavelengths) was measured at a low temperature (77 [K]), a tangent line was drawn to the rise on the short wavelength side of this phosphorescence spectrum, and the energy amount calculated from the following conversion formula (F1) based on the wavelengths λ_{edge} [nm] at the intersection of the tangent line and the horizontal axis was taken as the triplet energy T₁.

$${T}_{1} [ eV ] = 1239.85 / {λ}_{edge} \qquad Conversion formula ( F1 )$$

The tangent line to the rise on the short wavelength side of the phosphorescence spectrum was drawn as follows. When moving on the spectrum curve from the short wavelength side of the phosphorescence spectrum to the maximum value on the shortest wavelength side among the maximum values of the spectrum, the tangent line at each point on the curve is considered toward the long wavelength side. This tangent line increases in slope as the curve rises (i.e., as the vertical axis increases). A tangent line drawn at a point at which the slope has a maximum value (i.e., a tangent line at the inflection point) was regarded as a tangent line to the rise of the phosphorescence spectrum on the short wavelength side.

Note that a maximum point having a peak intensity of 15% or less of the maximum peak intensity of the spectrum was not included in the above-described maximum value on the shortest wavelength side, and a tangent line drawn at a point which is closest to the maximum value on the shortest wavelength side and at which the value of the slope is a maximum value was regarded as a tangent line to the rise on the short wavelength side of the phosphorescence spectrum.

For the measurement of phosphorescence, an F-4500 type fluorescence spectrophotometer main body manufactured by Hitachi High-Tech Corporation was used.

### (Refractive Index, Extinction Coefficient, and Order Parameter)

A refractive index, an extinction coefficient, and an order parameter of a constituent material (compound or composition) constituting the organic layer and a compound serving as a reference material were measured and calculated as follows.

A material to be measured was vacuum-deposited on a glass substrate to a film thickness of about 50 nm to prepare a sample to be measured, and the sample was irradiated with incident light (ultraviolet to visible light to near-infrared light) every 5° in a measurement angle range of 45° to 75° with a spectroscopic ellipsometry device (M-2000UI, manufactured by J. A. Woollam Company, Inc., USA) to measure a change in a polarization state of light reflected from the sample surface. In order to increase the measurement accuracy of the extinction coefficient, the transmission spectrum in the substrate normal direction (direction perpendicular to the surface of the organic EL device substrate) was also measured by the device. Similarly, only the glass substrate on which the material to be measured was not vapor-deposited was subjected to the same measurement. The obtained measurement information was subjected to fitting with analysis software (Complete EASE) manufactured by J. A. Woollam Company, Inc.

As the fitting conditions, the refractive indices in the in-plane direction and the normal direction of the organic film formed on the substrate, the extinction coefficients in the in-plane direction and the normal direction, and the order parameter were calculated using a uniaxial rotationally symmetric anisotropic model such that the parameter MSE indicating the mean square error in the software was 3.0 or less. The order parameter was calculated from the peak wavelengths of the S1, with the peak on the long wavelength side of the extinction coefficient (in-plane direction) as the S1. As the fitting condition for the glass substrate, an isotropic model was used.

The film of the low-molecular-weight material vacuum-deposited on the substrate usually has uniaxial rotational symmetry with the substrate normal direction as the rotation target axis. In the case where an angle formed between a molecular axis in a thin film formed on a substrate and a substrate normal direction is denoted by θ, and extinction coefficients in a substrate parallel direction (Ordinary direction) and a substrate perpendicular direction (Extra-Ordinary direction) obtained by multiple incident angle spectroscopic ellipsometry measurement of the thin film are denoted by ko and ke, respectively, S′ represented by the following equations is an order parameter.

$${S}^{′} = 1 - 〈 cos 2 θ 〉 = 2 ko / ( ke + 2 ko ) = 2 / 3 ( 1 - S ) \\ S = ( 1 / 2 ) 〈 3 cos 2 θ - 1 〉 = ( ke - ko ) / ( ke + 2 ko )$$

The method for forming the thin film was a vacuum deposition method.

The order parameter S′ obtained from the multiple incident angle spectroscopic ellipsometry measurement is 1.0 when all the molecules are oriented in the direction parallel to the substrate. The order parameter S′ is 0.66 when the molecules are not oriented and are random.

Table 2 shows the measurement results of Compound 1 and Comparative Compounds 1 to 3.

TABLE 2

| | HOMO | LUMO | T₁ | 80% Attenuation |
|---|---|---|---|---|
| Compound | [eV] | [eV] | [eV] | time t of PL [h] |
| Compound 1 | −5.75 | −1.63 | 2.23 | 2.7 |
| Comparative | −5.82 | −1.39 | 2.69 | 0.05 |
| Compound 1 | | | | |
| Comparative | −5.75 | −1.90 | 2.09 | 44 |
| Compound 2 | | | | |
| Comparative | −5.67 | −1.36 | 2.87 | 0.09 |
| Compound 3 | | | | |

As shown in Table 2, regarding Compound 1, the highest occupied molecular orbital energy level HOMO satisfies the condition (A), the triplet energy T₁ satisfies the condition (B), and the 80% attenuation time t of the photoluminescence intensity PL satisfies the condition (C).

On the other hand, regarding Comparative Compound 1, the highest occupied molecular orbital energy level HOMO satisfies the condition (A), and the triplet energy T₁ satisfies the condition (B), but the 80% attenuation time t of the photoluminescence intensity PL is extremely short, and the condition (C) is not satisfied.

In addition, in Comparative Compound 2, the highest occupied molecular orbital energy level HOMO satisfies the condition (A), and the 80% attenuation time t of the photoluminescence intensity PL satisfies the condition (C), but T₁ is very small, and the condition (B) is not satisfied.

In addition, in Comparative Compound 3, the highest occupied molecular orbital energy level HOMO satisfies the condition (A), but the 80% attenuation time t of the photoluminescence intensity PL is extremely short, and the condition (C) is not satisfied.

Compounds used for Production of Organic EL Devices of Examples 6 to 11

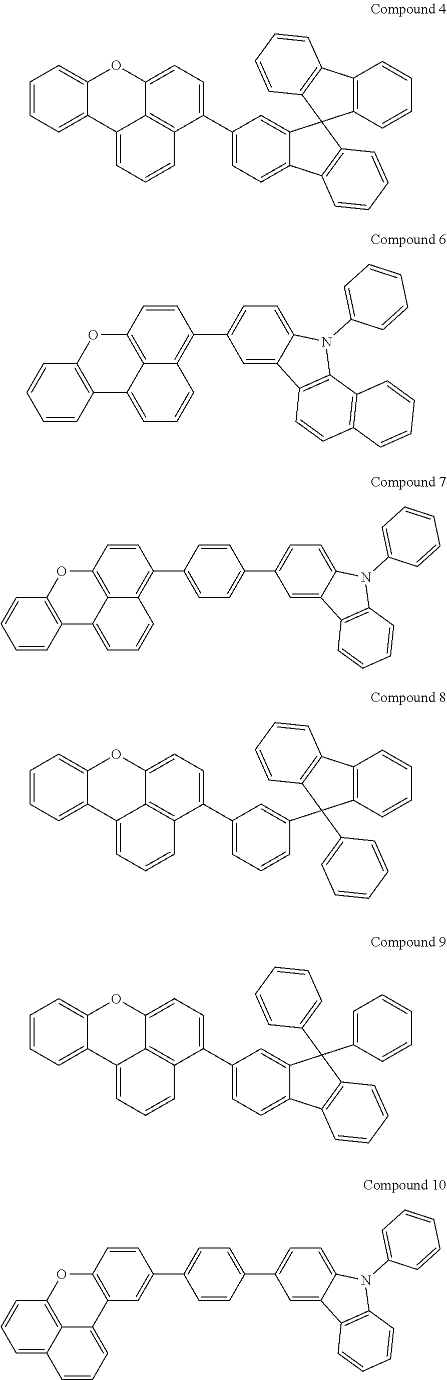 [SMILES: c1ccc2c(c1)Oc1ccc(-c3ccc4c(c3)C3(c5ccccc5-c5ccccc53)c3ccccc3-4)c3cccc-2c13 ; c1ccc(-n2c3ccc(-c4ccc5c6c(cccc46)-c4ccccc4O5)cc3c3ccc4ccccc4c32)cc1 ; c1ccc(-n2c3ccccc3c3cc(-c4ccc(-c5ccc6c7c(cccc57)-c5ccccc5O6)cc4)ccc32)cc1 ; c1ccc(C2(c3cccc(-c4ccc5c6c(cccc46)-c4ccccc4O5)c3)c3ccccc3-c3ccccc32)cc1 ; c1ccc(C2(c3ccccc3)c3ccccc3-c3ccc(-c4ccc5c6c(cccc46)-c4ccccc4O5)cc32)cc1 ; c1ccc(-n2c3ccccc3c3cc(-c4ccc(-c5ccc6c(c5)-c5cccc7cccc(c57)O6)cc4)ccc32)cc1]

Comparative Compounds used for Production of Organic EL Devices of Comparative Examples 4 and 5

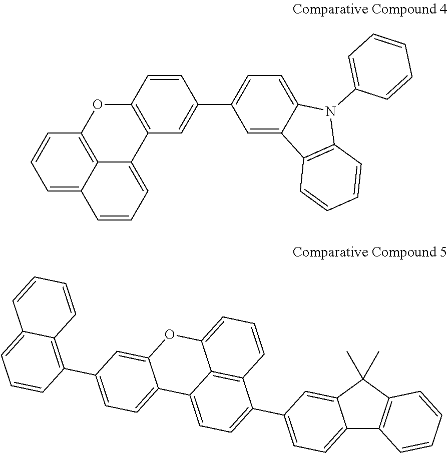 [SMILES: c1ccc(-n2c3ccccc3c3cc(-c4ccc5c(c4)-c4cccc6cccc(c46)O5)ccc32)cc1 ; CC1(C)c2ccccc2-c2ccc(-c3ccc4c5c(cccc35)Oc3cc(-c5cccc6ccccc56)ccc3-4)cc21]

Other Compounds used for Production of Organic EL Devices of Examples 6 to 11 and Comparative Examples 4 and 5

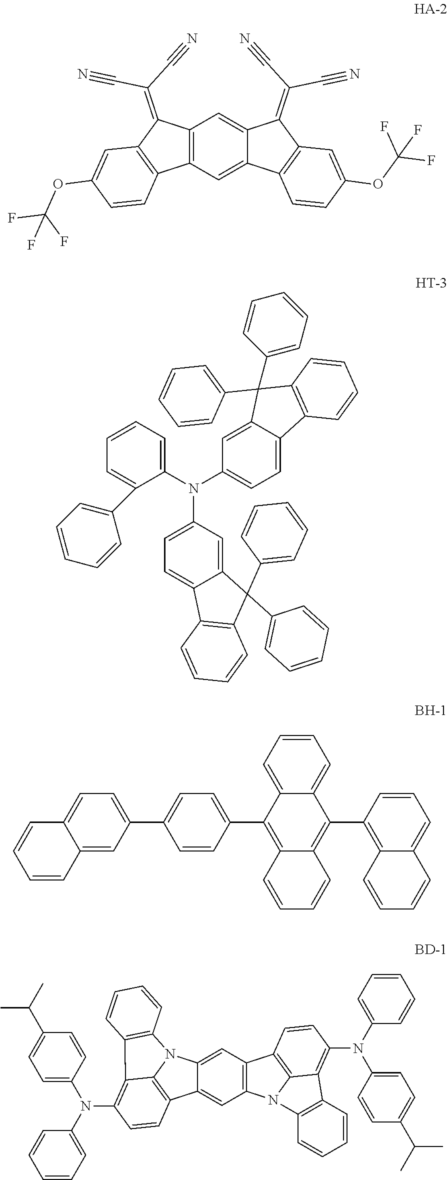

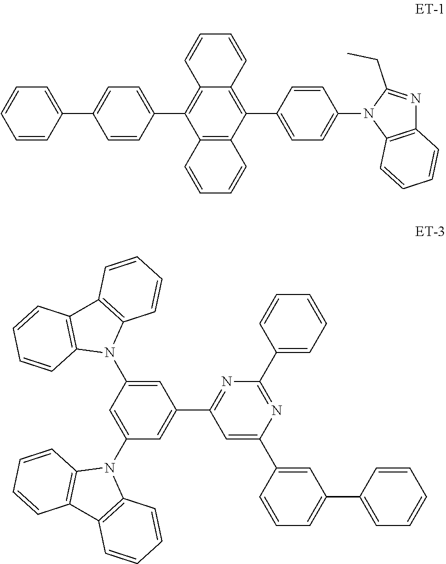 [SMILES: CCc1nc2ccccc2n1-c1ccc(-c2c3ccccc3c(-c3ccc(-c4ccccc4)cc3)c3ccccc23)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

### Production of Organic EL Devices 2

### Example 6

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the ITO transparent electrode was mounted on a substrate holder of a vacuum vapor deposition apparatus, and firstly, Compound HA-2 was vapor deposited on the surface having the transparent electrode formed thereon, so as to cover the transparent electrode, resulting in a hole injecting layer with a film thickness of 5 nm.

Subsequently, on this hole injecting layer, Compound HT-3 was vapor deposited to form a first hole transporting layer with a film thickness of 80 nm.

Subsequently, Compound 4 as Compound HT-2 (second hole transporting material) was vapor deposited on the first hole transporting layer to form a second hole transporting layer with a film thickness of 10 nm.

Subsequently, on this second hole transporting layer, Compound BH-1 (host material) and Compound BD-1 (dopant material) were vapor co-deposited to form a light emitting layer with a film thickness of 25 nm. The mass ratio of Compound BH-1 to Compound BD-1 (BH-1:BD-1) was 98:2.

Subsequently, on this light emitting layer, Compound ET-3 was vapor deposited to form a first electron transporting layer with a film thickness of 10 nm.

Subsequently, on this first electron transporting layer, Compound ET-1 was vapor deposited to form a second electron transporting layer.

Next, LiF was vapor deposited on this second electron transporting layer to form an electron injecting electrode having a film thickness of 1 nm.

Next, metal A1 was vapor deposited on the electron injecting electrode to form a metal cathode having a film thickness of 50 nm.

The layer configuration of the organic EL device of Example 6 thus obtained is shown below.

$$ITO ( 130 ) / HA - 2 ( 5 ) / HT - 3 ( 80 ) / HT - 2 ( 10 ) / BH - 1 : BD - 1 = 98 : 2 ( 25 ) / ET - 3 ( 10 ) / ET - 1 ( 15 ) / LiF ( 1 ) / Al ( 80 )$$

In the layer configuration, the numeral in parentheses indicates the film thickness (nm), and the ratio is a mass ratio.

### Examples 6 to 11

Organic EL devices were produced in the same manner as in Example 6 except that the second hole transporting layer material HT-2 was changed from Compound 4 to Compounds 6 to 10, respectively, as shown in Table 3 below.

### Comparative Examples 4 and 5

Organic EL devices were produced in the same manner as in Example 6 except that the second hole transporting layer material HT-2 was changed from Compound 4 to Comparative Compounds 4 and 5, respectively, as shown in Table 3 below.

### Evaluation of Organic EL Devices 2

For the organic EL devices produced in Examples 6 to 11 and Comparative Examples 4 and 5, a voltage was applied to the organic EL device so that the current density was 10 mA/cm², and the external quantum efficiency (EQE) was evaluated.

In addition, for each of the organic EL devices described above, a voltage was applied to the organic EL device so that the current density was 50 mA/cm², and LT95 (hr) was evaluated.

The results are shown in Table 3.

TABLE 3

| | Second hole | EQE | LT95 [h] |
|---|---|---|---|
| | transporting | @10 | @50 |
| | material (HT-2) | mA/cm² | mA/cm² |
| Example 6 | Compound 4 | 9.5 | 126 |
| Example 7 | Compound 6 | 9.9 | 79 |
| Example 8 | Compound 7 | 9.5 | 149 |
| Example 9 | Compound 8 | 9.6 | 65 |
| Example 10 | Compound 9 | 9.6 | 130 |
| Example 11 | Compound 10 | 9.6 | 146 |
| Comparative | Comparative | 9.2 | 58 |
| Example 4 | Compound 4 | | |
| Comparative | Comparative | 7.6 | 40 |
| Example 5 | Compound 5 | | |

As is clear from the results of Table 3, it is found that the compounds satisfying the requirements of the present invention and included in the above formula (1) (Compounds 4 and 6 to 10 of Examples 6 to 11) exhibit remarkably improved values of EQE and LT95 as compared with the monoamines not satisfying the requirements of the present invention and not included in the above formula (1) (Comparative Compounds 4 and 5 of Comparative Examples 4 and 5).

Compounds used for Production of Organic EL Devices of Examples 12 and 13

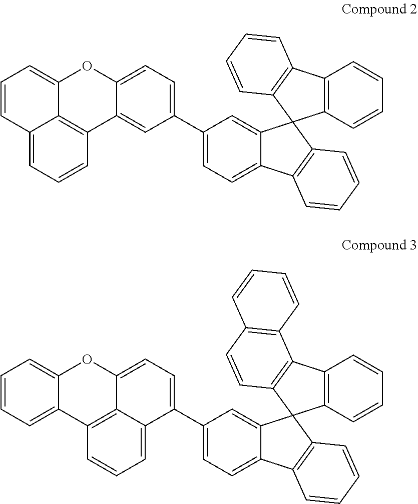 [SMILES: c1ccc2c(c1)-c1ccccc1C21c2ccccc2-c2ccc(-c3ccc4c(c3)-c3cccc5cccc(c35)O4)cc21 ; c1ccc2c(c1)Oc1ccc(-c3ccc4c(c3)C3(c5ccccc5-4)c4ccccc4-c4c3ccc3ccccc43)c3cccc-2c13]

Comparative Compound used for Production of Organic EL device of Comparative Example 6

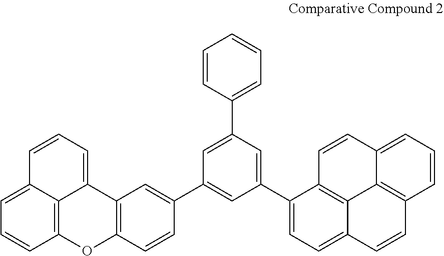 [SMILES: c1ccc(-c2cc(-c3ccc4c(c3)-c3cccc5cccc(c35)O4)cc(-c3ccc4ccc5cccc6ccc3c4c56)c2)cc1]

Other Compounds used for Production of Organic EL Devices of Examples 12 and 13 and Comparative Example 6

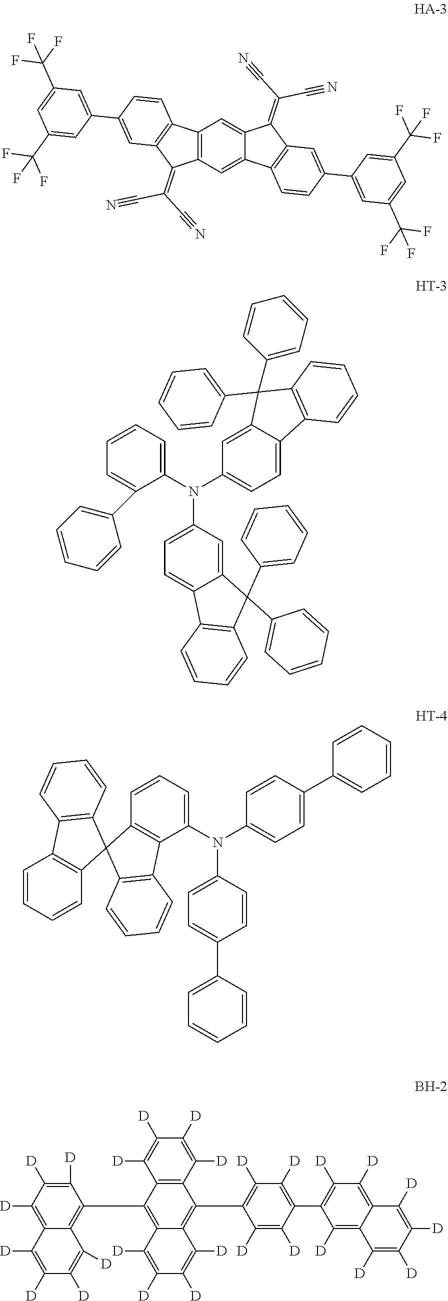

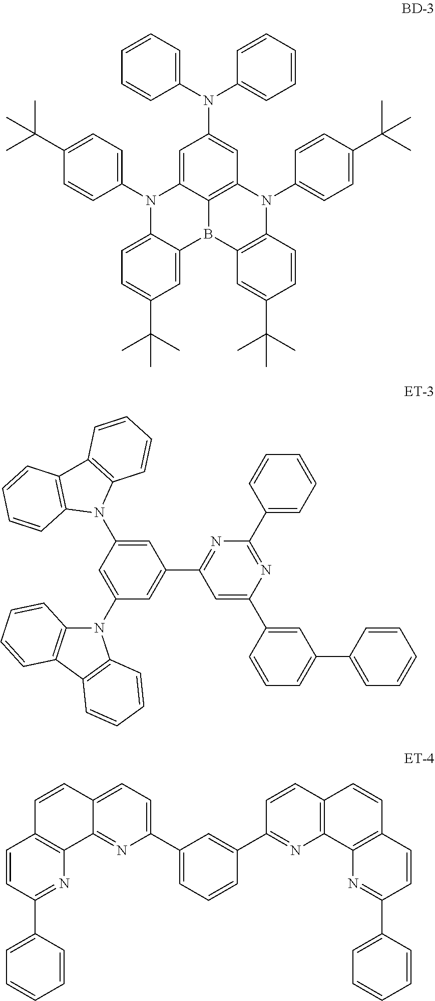 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2ccc3ccc4ccc(-c5cccc(-c6ccc7ccc8ccc(-c9ccccc9)nc8c7n6)c5)nc4c3n2)cc1]

### Production of Organic EL Devices 3

### Example 12

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the ITO transparent electrode was mounted on a substrate holder of a vacuum vapor deposition apparatus, and firstly, Compound HA-3 was vapor deposited on the surface having the transparent electrode formed thereon, so as to cover the transparent electrode, resulting in a hole injecting layer with a film thickness of 5 nm.

Subsequently, on this hole injecting layer, Compound HT-3 was vapor deposited to form a first hole transporting layer with a film thickness of 75 nm.

Subsequently, Compound HT-4 as a second hole transporting material was vapor deposited on the first hole transporting layer to form a second hole transporting layer with a film thickness of 10 nm.

Subsequently, Compound 2 and Compound BD-3 (first dopant material) were vapor co-deposited on the second hole transporting layer by using Compound 2 as a first host material BH, thereby forming a first light emitting layer with a film thickness of 6 nm. The mass ratio of the first host material BH to Compound BD-3 (BH:BD-3) was 98:2.

Subsequently, on this first light emitting layer, Compound BH-2 (second host material) and Compound BD-3 (second dopant material) were vapor co-deposited to form a second light emitting layer with a film thickness of 14 nm. The mass ratio of Compound BH-2 to Compound BD-3 (BH-2:BD-3) was 98:2.

Subsequently, on this second light emitting layer, Compound ET-3 was vapor deposited to form an electron transporting layer with a film thickness of 10 nm.

Subsequently, on this electron transporting layer, Compound ET-4 and metal L¹ were vapor co-deposited to form an electron injecting layer with a film thickness of 15 nm. The mass ratio of Compound ET-4 to L¹ (ET-4:L¹) was 96:4.

Next, metal A1 was vapor deposited on the electron injecting layer to form a metal cathode having a film thickness of 50 nm.

The layer configuration of the organic EL device of Example 12 thus obtained is shown below.

$$ITO ( 130 ) / HA - 3 ( 5 ) / HT - 3 ( 75 ) / HT - 4 ( 10 ) / BH : BD - 3 = 98 : 2 ( 6 ) / BH - 2 : BD - 3 = 98 : 2 ( 14 ) / ET - 3 ( 10 ) / ET - 4 : Li = 96 : 4 ( 15 ) / Al ( 50 )$$

In the layer configuration, the numeral in parentheses indicates the film thickness (nm), and the ratio is a mass ratio.

### Example 13

An organic EL device was produced in the same manner as in Example 12 except that the first host material BH was changed from Compound 2 to Compound 3 as shown in Table 4 below.

### Comparative Example 6

An organic EL device was produced in the same manner as in Example 12 except that the first host material BH was changed from Compound 2 to Comparative Compound 2 as shown in Table 4 below.

### Evaluation of Organic EL Devices 3

For the organic EL devices produced in Examples 12 and 13 and Comparative Example 6, a voltage was applied to the organic EL device so that the current density was 10 mA/cm², and the external quantum efficiency (EQE) was evaluated.

In addition, for each of the organic EL devices described above, a voltage was applied to the organic EL device so that the current density was 50 mA/cm², and LT95 (hr) was evaluated.

The results are shown in Table 4.

TABLE 4

| | Second hole | First host | EQE | LT95 [h] |
|---|---|---|---|---|
| | transporting | material | @10 | @50 |
| | material | (BH) | mA/cm² | mA/cm² |
| Example 12 | HT-4 | Compound 2 | 9.0 | 72 |
| Example 13 | HT-4 | Compound 3 | 9.3 | 121 |
| Example 14 | HT-5 | Compound 3 | 9.1 | 140 |
| Comparative | HT-4 | Comparative | 9.0 | 51 |
| Example 6 | | Compound 2 | | |

As is clear from the results of Table 4, it is found that the organic EL devices using the compounds satisfying the requirements of the present invention and included in the above formula (1) (Compounds 2 and 3 of Examples 12, 13 and 14) as the host material of the light emitting layer also exhibit remarkably improved values of EQE and LT95 as compared with the organic EL device using the monoamine not satisfying the requirements of the present invention and not included in the above formula (1) (Comparative Compound 2 of Comparative Example 6).

### <Synthesis of Compound>

### Synthesis Example 1: Synthesis of Compound 1

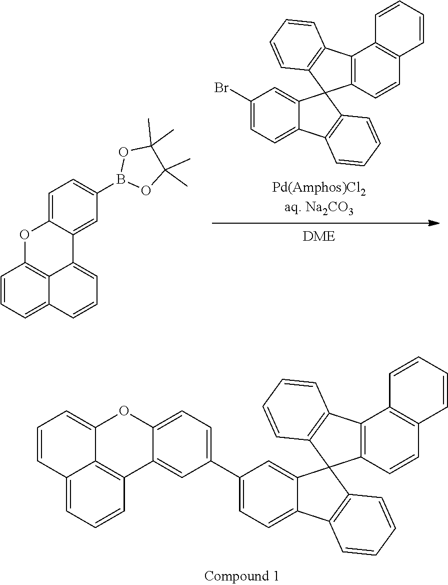 [SMILES: CC1(C)OB(c2ccc3c(c2)-c2cccc4cccc(c24)O3)OC1(C)C ; Brc1ccc2c(c1)C1(c3ccccc3-2)c2ccccc2-c2c1ccc1ccccc21 ; c1ccc2c(c1)-c1ccc(-c3ccc4c(c3)-c3cccc5cccc(c35)O4)cc1C21c2ccccc2-c2c1ccc1ccccc21]

Under an argon atmosphere, a mixture of 2.17 g (6.30 mmol) of 10-(4,4,5,5-tetramethyl-1,3,2-dioxaboran-2-yl)benzo[k1]xanthene, 2.67 g (6.0 mmol) of 2′-bromospiro[benzo[c]fluorene-7,9′-fluorene], 0.085 g (0.120 mmol) of bis(di-tert-butyl(4-dimethylaminophenyl)phosphine)dichloropalladium (II), 9.0 mL aqueous solution of 2M sodium carbonate, and 30 mL of DME was stirred at 80° C. for 3 hours. The reaction solution was cooled to room temperature, filtered, and concentrated under reduced pressure. The obtained residue was purified by silica gel column chromatography and recrystallization to obtain 1.92 g of a white solid. The yield was 55%.

As a result of mass spectrum analysis, the obtained product was Compound 1, and m/e was 583 with respect to the molecular weight of 582.70.

### Synthesis Example 2: Synthesis of Compound 2

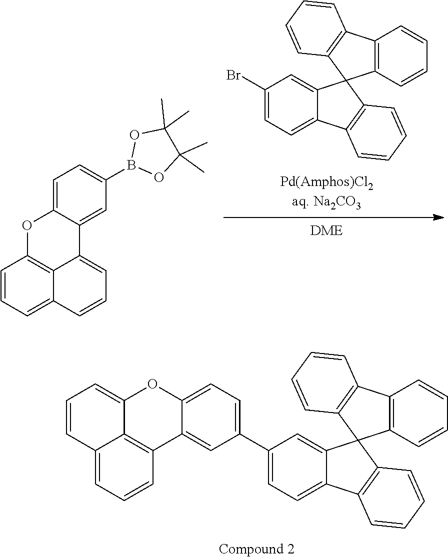 [SMILES: CC1(C)OB(c2ccc3c(c2)-c2cccc4cccc(c24)O3)OC1(C)C ; Brc1ccc2c(c1)C1(c3ccccc3-c3ccccc31)c1ccccc1-2 ; c1ccc2c(c1)-c1ccccc1C21c2ccccc2-c2ccc(-c3ccc4c(c3)-c3cccc5cccc(c35)O4)cc21]

A white solid was obtained by performing the same operation as in Synthesis Example 1, except that 2-bromo-9,9′-spirobi[9H-fluorene]was used instead of 2′-bromospiro[benzo[c]fluorene-7,9′-fluorene].

As a result of mass spectrum analysis, the obtained product was Compound 2, and m/e was 533 with respect to the molecular weight of 532.64.

### Synthesis Example 3: Synthesis of Compound 3

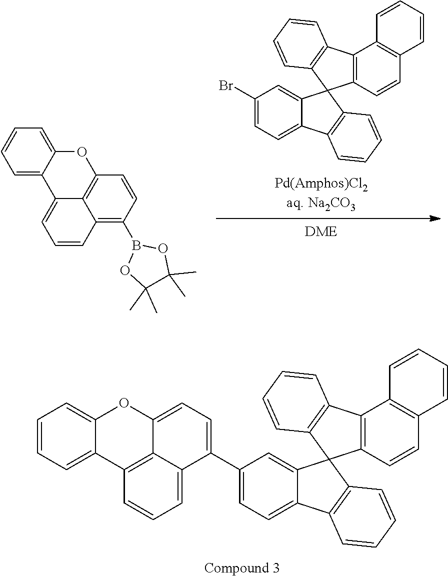 [SMILES: CC1(C)OB(c2ccc3c4c(cccc24)-c2ccccc2O3)OC1(C)C ; Brc1ccc2c(c1)C1(c3ccccc3-2)c2ccccc2-c2c1ccc1ccccc21 ; c1ccc2c(c1)Oc1ccc(-c3ccc4c(c3)C3(c5ccccc5-4)c4ccccc4-c4c3ccc3ccccc43)c3cccc-2c13]

A white solid was obtained by performing the same operation as in Synthesis Example 1, except that 4-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene. As a result of mass spectrum analysis, the obtained product was Compound 3, and m/e was 583 with respect to the molecular weight of 582.70.

### Synthesis Example 4: Synthesis of Compound 4

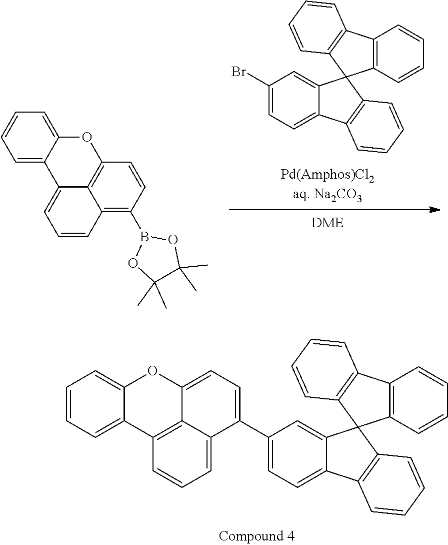 [SMILES: CC1(C)OB(c2ccc3c4c(cccc24)-c2ccccc2O3)OC1(C)C ; Brc1ccc2c(c1)C1(c3ccccc3-c3ccccc31)c1ccccc1-2 ; c1ccc2c(c1)Oc1ccc(-c3ccc4c(c3)C3(c5ccccc5-c5ccccc53)c3ccccc3-4)c3cccc-2c13]

A white solid was obtained by performing the same operation as in Synthesis Example 1, except that 4-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene, and 2-bromo-9,9′-spirobi[9H-fluorene]was used instead of 2′-bromospiro[benzo[c]fluorene-7,9′-fluorene].

As a result of mass spectrum analysis, the obtained product was Compound 4, and m/e was 533 with respect to the molecular weight of 532.64.

### Synthesis Example 5: Synthesis of Compound 5

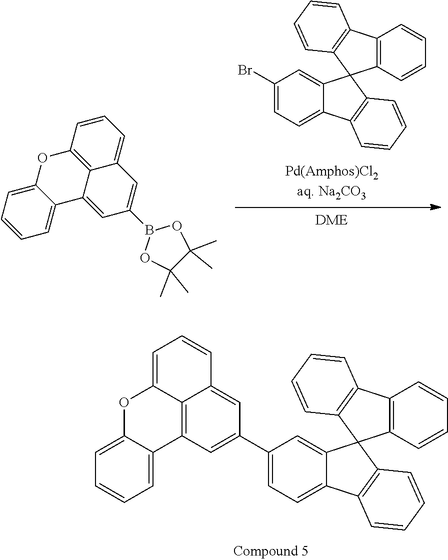 [SMILES: CC1(C)OB(c2cc3c4c(cccc4c2)Oc2ccccc2-3)OC1(C)C ; Brc1ccc2c(c1)C1(c3ccccc3-c3ccccc31)c1ccccc1-2 ; c1ccc2c(c1)Oc1cccc3cc(-c4ccc5c(c4)C4(c6ccccc6-c6ccccc64)c4ccccc4-5)cc-2c13]

A white solid was obtained by performing the same operation as in Synthesis Example 1, except that 2-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene, and 2-bromo-9,9′-spirobi[9H-fluorene]was used instead of 2′-bromospiro[benzo[c]fluorene-7,9′-fluorene].

As a result of mass spectrum analysis, the obtained product was Compound 5, and m/e was 533 with respect to the molecular weight of 532.64.

### Synthesis Example 6: Synthesis of Compound 6

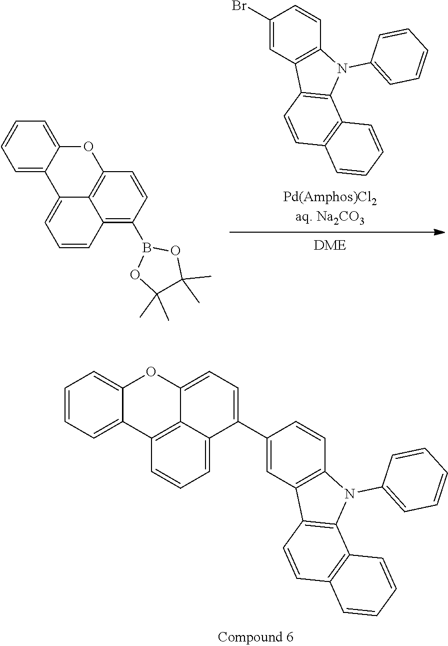 [SMILES: CC1(C)OB(c2ccc3c4c(cccc24)-c2ccccc2O3)OC1(C)C ; Brc1ccc2c(c1)c1ccc3ccccc3c1n2-c1ccccc1 ; c1ccc(-n2c3ccc(-c4ccc5c6c(cccc46)-c4ccccc4O5)cc3c3ccc4ccccc4c32)cc1]

A white solid was obtained by performing the same operation as in Synthesis Example 1, except that 4-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene, and 8-bromo-11-phenyl-11H-benzo[a]carbazole was used instead of 2′-bromospiro[benzo[c]fluorene-7,9′-fluorene].

As a result of mass spectrum analysis, the obtained product was Compound 6, and m/e was 510 with respect to the molecular weight of 509.61.

### Synthesis Example 7: Synthesis of Compound 7

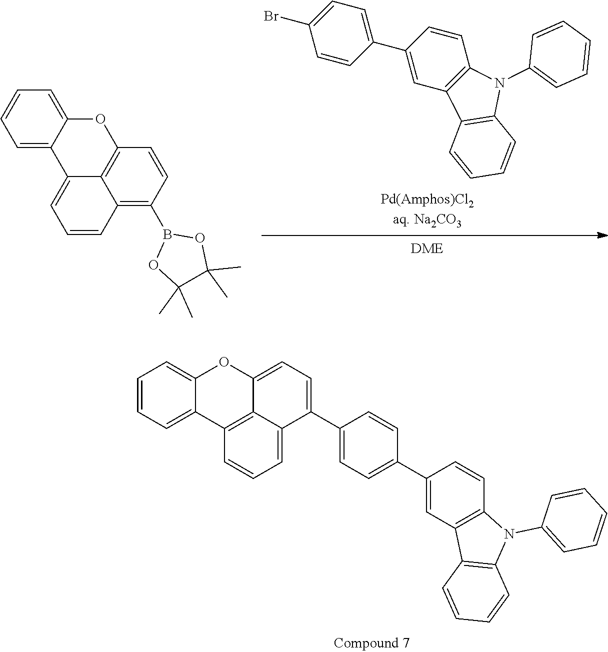 [SMILES: CC1(C)OB(c2ccc3c4c(cccc24)-c2ccccc2O3)OC1(C)C ; Brc1ccc(-c2ccc3c(c2)c2ccccc2n3-c2ccccc2)cc1 ; c1ccc(-n2c3ccccc3c3cc(-c4ccc(-c5ccc6c7c(cccc57)-c5ccccc5O6)cc4)ccc32)cc1]

A white solid was obtained by performing the same operation as in Synthesis Example 1, except that 4-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene, and 3-(4-bromophenyl)-9-phenyl-9H-carbazole was used instead of 2′-bromospiro[benzo[c]fluorene-7,9′-fluorene].

As a result of mass spectrum analysis, the obtained product was Compound 7, and m/e was 536 with respect to the molecular weight of 535.65.

### Synthesis Example 8: Synthesis of Compound 8

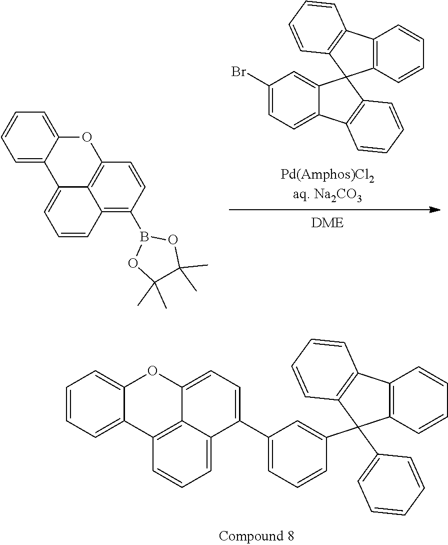 [SMILES: CC1(C)OB(c2ccc3c4c(cccc24)-c2ccccc2O3)OC1(C)C ; Brc1ccc2c(c1)C1(c3ccccc3-c3ccccc31)c1ccccc1-2 ; c1ccc(C2(c3cccc(-c4ccc5c6c(cccc46)-c4ccccc4O5)c3)c3ccccc3-c3ccccc32)cc1]

A white solid was obtained by performing the same operation as in Synthesis Example 1, except that 4-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene, and 9-(3-bromophenyl)-9-phenyl-9H-fluorene was used instead of 2′-bromospiro[benzo[c]fluorene-7,9′-fluorene].

As a result of mass spectrum analysis, the obtained product was Compound 8, and m/e was 535 with respect to the molecular weight of 534.66.

### Synthesis Example 9: Synthesis of Compound 9

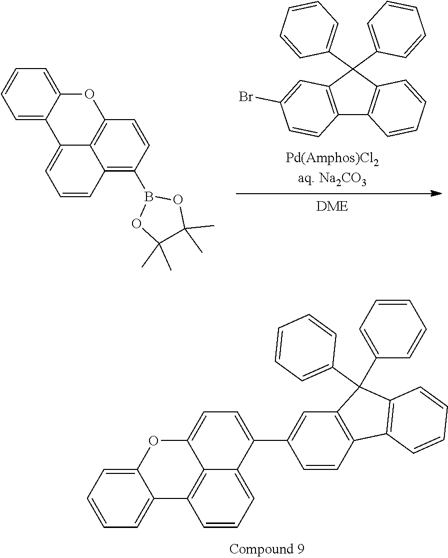 [SMILES: CC1(C)OB(c2ccc3c4c(cccc24)-c2ccccc2O3)OC1(C)C ; Brc1ccc2c(c1)C(c1ccccc1)(c1ccccc1)c1ccccc1-2 ; c1ccc(C2(c3ccccc3)c3ccccc3-c3ccc(-c4ccc5c6c(cccc46)-c4ccccc4O5)cc32)cc1]

A white solid was obtained by performing the same operation as in Synthesis Example 1, except that 4-(4,4,5,5-tetramethyl-1,3,2-dioxaboran-2-yl)benzo[k1]xanthene was used instead of 10-(4,4,5,5-tetramethyl-1,3,2-dioxaboran-2-yl)benzo[k1]xanthene, and 2-bromo-9,9-diphenyl-9H-fluorene was used instead of 2′-bromospiro[benzo[c]fluorene-7,9′-fluorene].

As a result of mass spectrum analysis, the obtained product was Compound 9, and m/e was 535 with respect to the molecular weight of 534.66.

### Synthesis Example 10: Synthesis of Compound 10

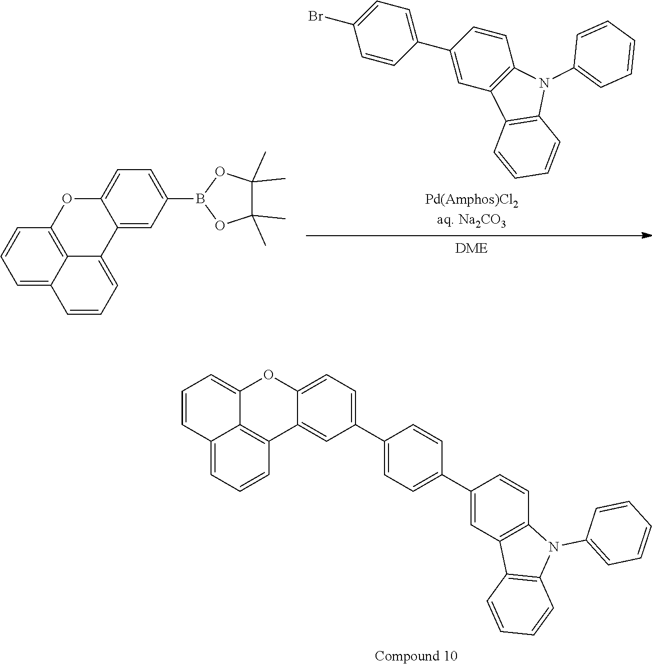 [SMILES: CC1(C)OB(c2ccc3c(c2)-c2cccc4cccc(c24)O3)OC1(C)C ; Brc1ccc(-c2ccc3c(c2)c2ccccc2n3-c2ccccc2)cc1 ; c1ccc(-n2c3ccccc3c3cc(-c4ccc(-c5ccc6c(c5)-c5cccc7cccc(c57)O6)cc4)ccc32)cc1]

A white solid was obtained by performing the same operation as in Synthesis Example 1, except that 3-(4-bromophenyl)-9-phenyl-9H-carbazole was used instead of 2′-bromospiro[benzo[c]fluorene-7,9′-fluorene].

As a result of mass spectrum analysis, the obtained product was Compound 10, and m/e was 536 with respect to the molecular weight of 535.65.

### Synthesis Example 11: Synthesis of Compound HT-5

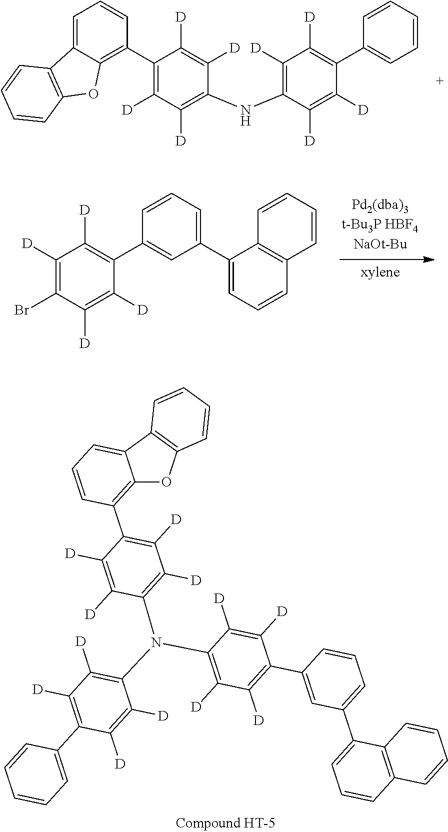 [SMILES: [2H]c1c([2H])c(-c2ccccc2)c([2H])c([2H])c1Nc1c([2H])c([2H])c(-c2cccc3c2oc2ccccc23)c([2H])c1[2H] ; [2H]c1c([2H])c(-c2cccc(-c3cccc4ccccc34)c2)c([2H])c([2H])c1Br ; [2H]c1c([2H])c(N(c2c([2H])c([2H])c(-c3cccc(-c4cccc5ccccc45)c3)c([2H])c2[2H])c2c([2H])c([2H])c(-c3cccc4c3oc3ccccc34)c([2H])c2[2H])c([2H])c([2H])c1-c1ccccc1]

Under an argon atmosphere, a mixture of 3.63 g (10.0 mmol) of 4′-bromo-3-(naphthalen-1-yl)(2′,3′,5′,6′-d4)-1,1′-biphenyl, 4.20 g (10.0 mmol) of N-[4-(dibenzo[b,d]furan-4-yl)(2,3,5,6-d4)phenyl](2,3,5,6-d4)[1,1′-biphenyl]-4-amine, 0.183 g (0.200 mmol) of tris(dibenzylideneacetone)dipalladium(0), 0.232 g (0.800 mmol) of tri-t-butylphosphonium tetrafluoroborate, 1.35 g (14.0 mmol) of sodium-t-butoxide, and 67 mL of xylene was refluxed at boiling point for 7 hours. The reaction solution was cooled to room temperature, and concentrated under reduced pressure. The obtained residue was purified by silica gel column chromatography and recrystallization to obtain 3.59 g of a white solid. The yield was 51%.

As a result of mass spectrum analysis, the obtained product was Compound HT-5, and m/e was 702 with respect to the molecular weight of 701.93.

### REFERENCE SIGNS LIST

1, 11, 12: Organic EL device
2: Substrate
3: Anode
4: Cathode
5: Light emitting layer
6: Hole transporting zone (hole transporting layer)
6a: Hole injecting layer
6b: First hole transporting layer
6c: Second hole transporting layer
6d: Third hole transporting layer
7: Electron transporting zone (electron transporting layer)
7a: First electron transporting layer
7b: Second electron transporting layer
10, 20, 30: Light emitting unit

1: A compound represented by the following formula (1):
Ar¹—Ar² (1)
where,
Ar¹ is represented by the following formula (1Aa), (1Ab), or (1B): 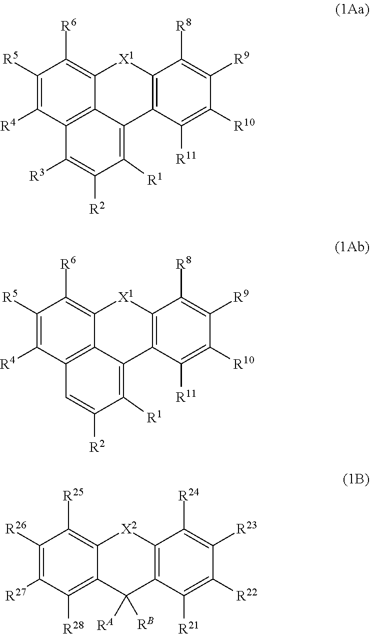
where in the formulae (1A) and (1B),
X¹ is an oxygen atom or a sulfur atom,
R¹ to R⁶ and R⁸ to R¹¹ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
one of R¹ to R⁶ and R⁸ to R¹¹ which is not a hydrogen atom is a single bond bonded to Ar² or a group bonded to Ar², and
a pair of groups adjacent to each other among R¹ to R⁶ and R⁸ to R¹¹ which are not a hydrogen atom and which are not a single bond are bonded to each other to form a substituted or unsubstituted monocyclic ring, are bonded to each other to form a substituted or unsubstituted condensed ring, or are not bonded to each other and do not form a ring,
where in the formula (1B),
X² is an oxygen atom or a sulfur atom,
R²¹ to R²⁸ are each independently a hydrogen atom, a substituted or unsubstituted alkyl group having 1 to 6 carbon atoms, a substituted or unsubstituted aryl group having 6 to 12 ring carbon atoms, or a heteroaryl group having 5 to 13 ring atoms,
R^{A} and R^{B} are each independently a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R²¹ to R²⁸, R^{A}, and R^{B} is a single bond bonded to Ar² or a group bonded to Ar²,
a pair of groups adjacent to each other among R²¹ to R²⁴ and R²⁵ to R²⁸ which are not a single bond are bonded to each other to form a substituted or unsubstituted monocyclic ring, are bonded to each other to form a substituted or unsubstituted condensed ring, or are not bonded to each other and do not form a ring, and
R^{A} and R^{B} which are not a single bond are bonded to each other to form a substituted or unsubstituted monocyclic ring, are bonded to each other to form a substituted or unsubstituted condensed ring, or are not bonded to each other and do not form a ring,
Ar² is represented by the following formula (2A), (2B), (2C), (2D), (2E), or (2F), provided that when Ar¹ is represented by the formula (1Aa), Ar² is represented by the following formula (2C), (2D), (2E) or (2F); when Ar¹ is represented by the formula (1Ab), Ar² is represented by the following formula (2A) or (2B); and when Ar¹ is represented by the formula (1B), Ar² is represented by the following formula (2A), (2B), (2C), (2D), (2E) or (2F): 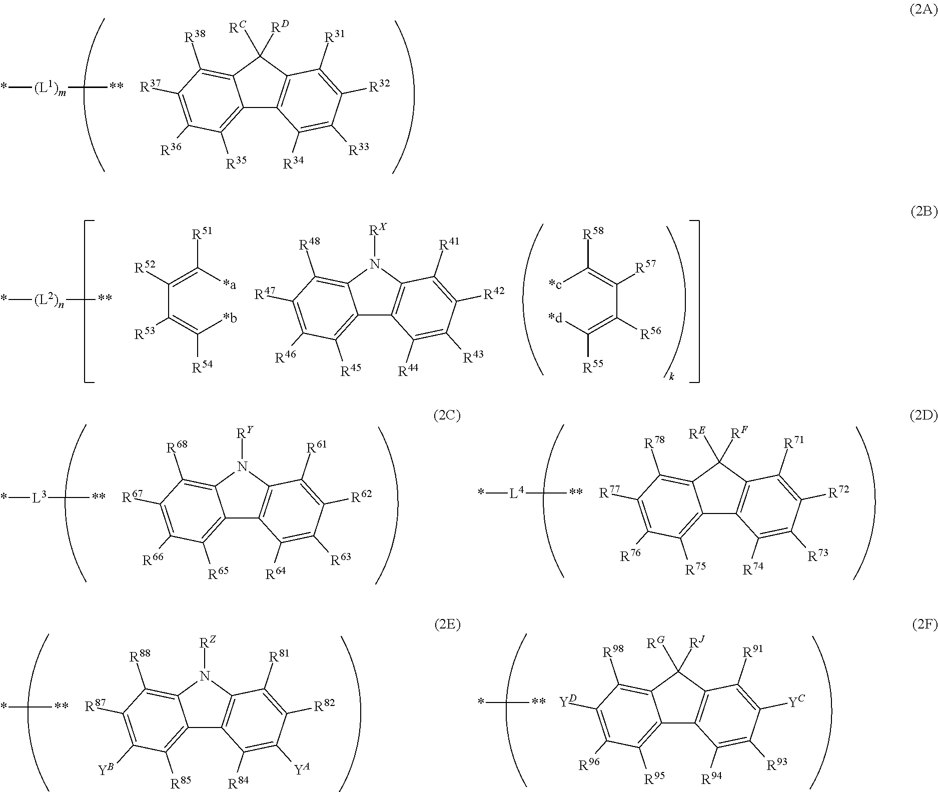
where in the formula (2A),
L¹ is a substituted or unsubstituted arylene group having 6 to 30 ring carbon atoms, or a substituted or unsubstituted divalent heterocyclic group having 5 to 30 ring atoms,
* represents one end of L¹ or a single bond bonded to Ar¹, and ** represents the other end of L¹ or a single bond,
m is 0 or 1,
R³¹ to R³⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R^{C} and R^{D} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R³¹ to R³⁸, R^{C}, and R^{D} is a single bond bonded to ** or a group bonded to **,
a pair of groups adjacent to each other among R³¹ to R³⁸ which are not a single bond are bonded to each other to form a substituted or unsubstituted monocyclic ring, are bonded to each other to form a substituted or unsubstituted condensed ring, or are not bonded to each other and do not form a ring, and
R^{C} and R^{D} which are not a single bond are bonded to each other to form a substituted or unsubstituted monocyclic ring, are bonded to each other to form a substituted or unsubstituted condensed ring, or are not bonded to each other and do not form a ring,
where in the formula (2B),
L² is a substituted or unsubstituted arylene group having 6 to 30 ring carbon atoms, or a substituted or unsubstituted divalent heterocyclic group having 5 to 30 ring atoms,
* represents one end of L² or a single bond bonded to Ar¹, and ** represents the other end of L² or the single bond,
n is 0 or 1,
R⁴¹ to R⁴⁸, R⁵¹ to R⁵⁴, and R⁵⁵ to R⁵⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
one of R⁴⁵ and R⁴⁶, R⁴⁶ and R⁴⁷, or R⁴⁷ and R⁴⁸ is a single bond bonded to *a, and the other is a single bond bonded to *b,
k is 0 or 1,
when k is 1, one of R⁴¹ and R⁴², R⁴² and R⁴³, or R⁴³ and R⁴⁴ is a single bond bonded to *c, and the other is a single bond bonded to *d,
R^{X} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R⁴¹ to R⁴⁴ which are not bonded to *c and *d, R⁴⁵ to R⁴⁸ which are not bonded to *a and *b, R⁵¹ to R⁵⁴, R⁵⁵ to R⁵⁸, and R^{X} is a single bond bonded to ** or a group bonded to **, and
a pair of groups adjacent to each other among R⁴¹ to R⁴⁴ which are not bonded to *c and *d and are not a single bond bonded to **, R⁴⁵ to R⁴⁸ which are not bonded to *a and *b and are not a single bond bonded to **, R⁵¹ to R⁵⁴, and R⁵⁵ to R⁵⁸ a bonded to each other to form a substituted or unsubstituted monocyclic ring, are bonded to each other to form a substituted or unsubstituted condensed ring, or are not bonded to each other and do not form a ring,
where in the formula (2C),
L³ is a substituted or unsubstituted arylene group having 6 to 30 ring carbon atoms, or a substituted or unsubstituted divalent heterocyclic group having 5 to 30 ring atoms,
* represents one end of L³ bonded to Ar¹, and ** represents the other end of L³,
R⁶¹ to R⁶⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R⁶¹ to R⁶⁸ are not bonded to each other and do not form a ring,
R^{Y} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and
one of R⁶¹ to R⁶⁸ and R^{Y} is a single bond bonded to ** or a group bonded to **,
where in the formula (2D),
L⁴ is a substituted or unsubstituted arylene group having 6 to 30 ring carbon atoms, or a substituted or unsubstituted divalent heterocyclic group having 5 to 30 ring atoms,
* represents one end of L⁴ bonded to Ar¹, and ** represents the other end of L⁴,
R⁷¹ to R⁷⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R^{E} and R^{F} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R⁷¹ to R⁷⁸, R^{E}, and R^{F} is a single bond bonded to ** or a group bonded to **,
a pair of groups adjacent to each other among R⁷¹ to R⁷⁸ which are not a single bond are bonded to each other to form a substituted or unsubstituted monocyclic ring, are bonded to each other to form a substituted or unsubstituted condensed ring, or are not bonded to each other and do not form a ring, and
R^{E} and R^{F} which are not a single bond are bonded to each other to form a substituted or unsubstituted monocyclic ring, are bonded to each other to form a substituted or unsubstituted condensed ring, or are not bonded to each other and do not form a ring,
where in the formula (2E),
* represents one end of a single bond bonded to Ar¹, and ** represents the other end of the single bond,
R⁸¹, R⁸², R⁸⁴, R⁸⁵, R⁸⁷, R⁸⁸, Y^{A}, and Y^{B} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R⁸¹, R⁸², R⁸⁴, R⁸⁵, R⁸⁷, R⁸⁸, Y^{A}, and Y^{B} are not bonded to each other and do not form a ring,
R^{Z} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and
one of R⁸¹, R⁸², R⁸⁴, R⁸⁵, R⁸⁷, R⁸⁸, and R^{Z} is a single bond bonded to ** or a group bonded to **,
where in the formula (2F),
* represents one end of a single bond bonded to Ar¹, and ** represents the other end of the single bond,
R⁹¹, R⁹³ to R⁹⁶, R⁹⁸, Y^{C}, and Y^{D} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R^{G} and R^{J} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R⁹¹, R⁹³ to R⁹⁶, R⁹⁸, R^{G}, and R^{Y} is a single bond bonded to ** or a group bonded to **,
a pair of groups adjacent to each other among R⁹¹, R⁹³ to R⁹⁶, R⁹⁸, Y^{C}, and Y^{D} which are not a single bond are bonded to each other to form a substituted or unsubstituted monocyclic ring, are bonded to each other to form a substituted or unsubstituted condensed ring, or are not bonded to each other and do not form a ring, and
R^{G} and R^{J} which are not a single bond are bonded to each other to form a substituted or unsubstituted monocyclic ring, are bonded to each other to form a substituted or unsubstituted condensed ring, or are not bonded to each other and do not form a ring.

2: The compound according to claim 1, represented by any one of the following formulae (1-1) to (1-4): 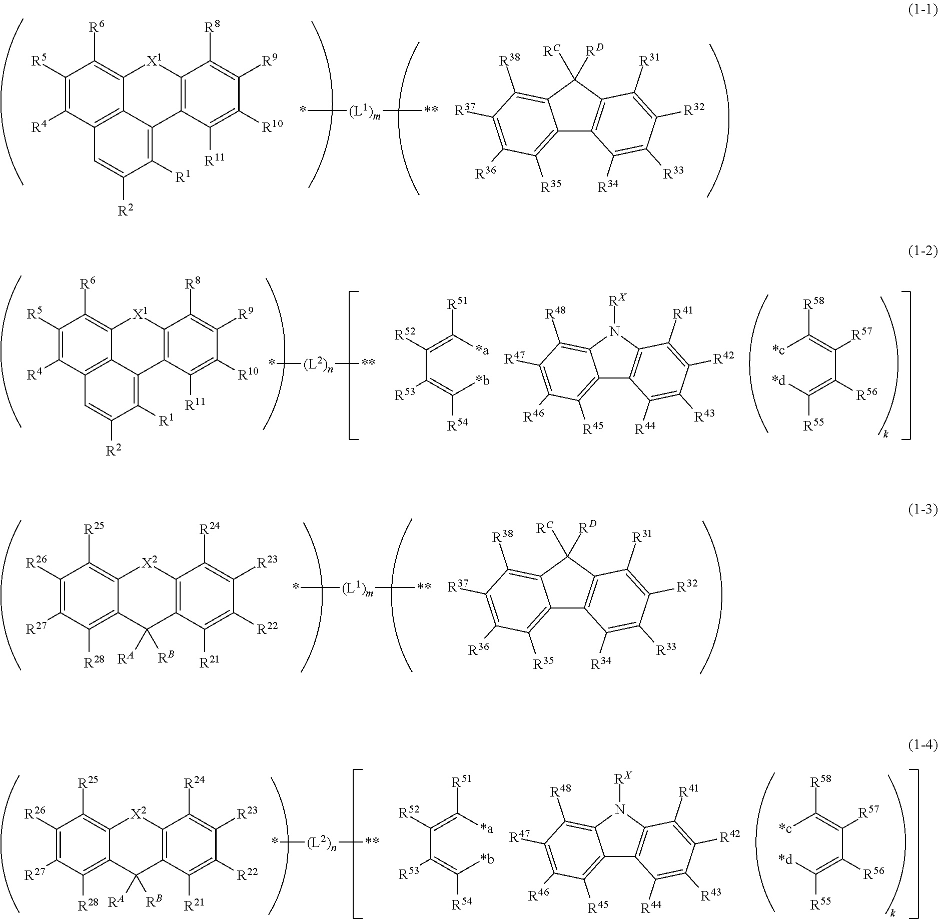
where X¹, X², L¹, L², R^{A}, R^{B}, R^{C}, R^{D}, R^{X}, R¹ to R⁶, R⁸ to R¹¹, R²¹ to R²⁸, R³¹ to R³⁸, R⁴¹ to R⁴⁸, R⁵¹ to R⁵⁴, R⁵⁵ to R⁵⁸, k, m, n, *, **, *a, *b, *c, and *d are as defined in the formula (1).

3-11. (canceled)

12: The compound according to claim 1, represented by any one of the following formulae (1-1-1), (1-2-1), (1-5-1), (1-6-1), (1-9-1), and (1-10-1): 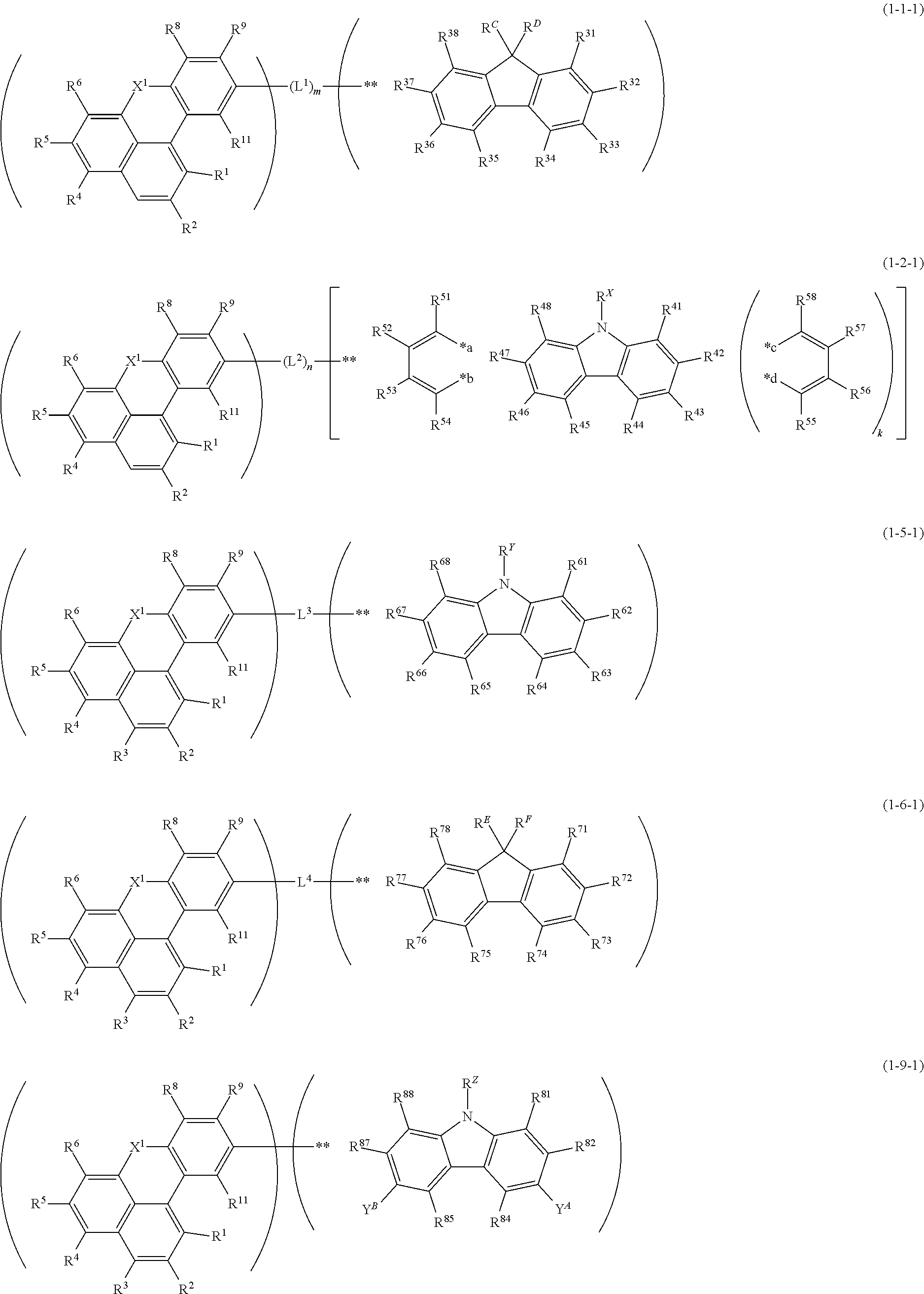 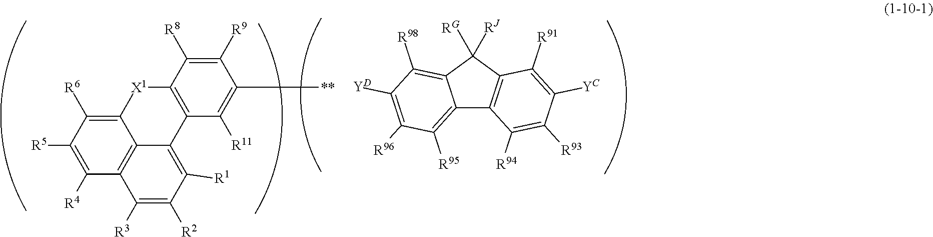
where X¹, L¹, L², L³, L⁴, R^{C}, R^{D}, R^{E}, R^{F}, R^{G}, R^{J}, R^{X}, R^{Y}, R^{Z}, R¹ to R⁶, R⁸ to R¹¹, R³¹ to R³⁸, R⁴¹ to R⁴⁸, R⁵¹ to R⁵⁴, R⁵⁵ to R⁵⁸, R⁶¹ to R⁶⁸, R⁷¹ to R⁷⁸, R⁸¹, R⁸², R⁸⁴, R⁸⁵, R⁸⁷, R⁸⁸, R⁹¹, R⁹³ to R⁹⁶, R⁹⁸, Y^{A}, Y^{B}, Y^{C}, Y^{D}, k, m, n, **, *a, *b, *c, and *d are as defined in the formula (1).

13: The compound according to claim 1, wherein m is 0, or m is 1 and L¹ is a phenylene group or a biphenylene group.

14: The compound according to claim 1, wherein n is 0, or n is 1 and L² is a phenylene group or a biphenylene group.

15-16. (canceled)

17: The compound according to claim 1, where
in the formula (2A), one of R³², R³⁴, R³⁵, and R³⁷ is a single bond bonded to ** or a group bonded to **;
in the formula (2D), one of R⁷², R⁷⁴, R⁷⁵, and R⁷⁷ is a single bond bonded to ** or a group bonded to **; and
in the formula (2F), one of R⁹⁴ and R⁹⁵ is a single bond bonded to ** or a group bonded to **.

18-20. (canceled)

21: The compound according to claim 1, wherein R^{C} and R^{D} are each independently selected from the group consisting of a phenyl group, a naphthyl group, and a phenanthryl group.

22: The compound according to claim 1, wherein R^{E} and R^{F} are each independently selected from the group consisting of a phenyl group, a naphthyl group, and a phenanthryl group.

23: The compound according to claim 1, wherein R^{G} and R^{J} are each independently selected from the group consisting of a phenyl group, a naphthyl group, and a phenanthryl group.

24-26. (canceled)

27: The compound according to claim 1, wherein Ar² represented by the formula (2B) is a group represented by any one of the following formulae (2Ba) to (2Bi): 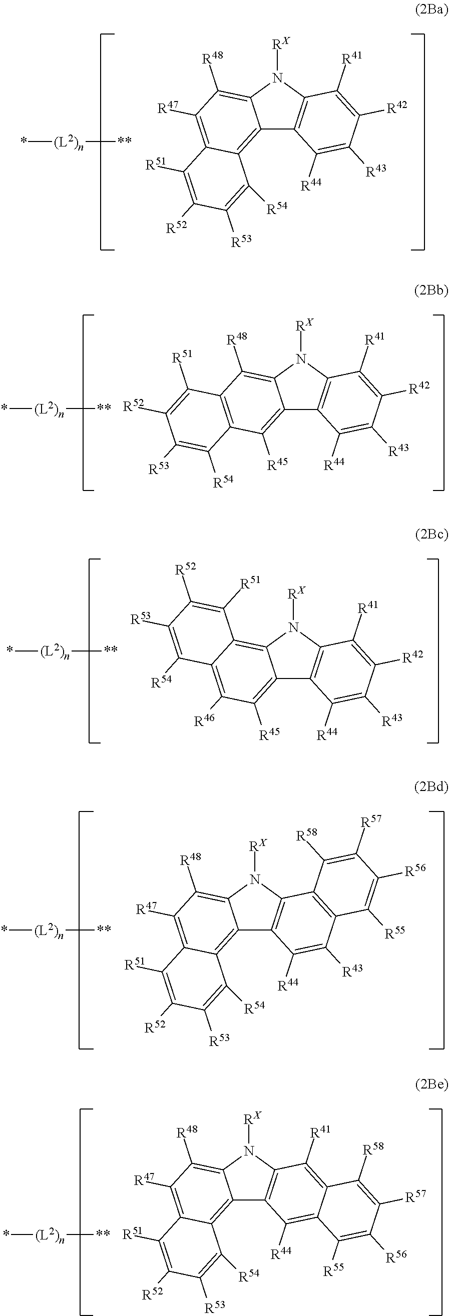 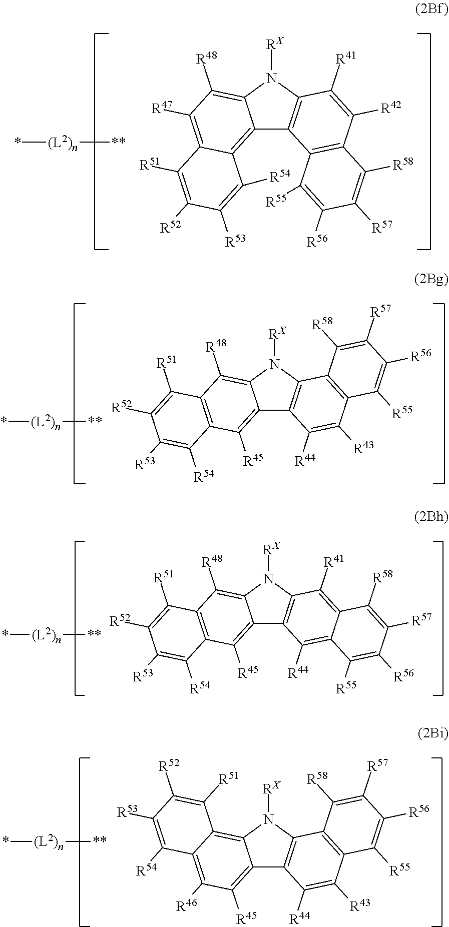
where L², R^{X}, R⁴¹ to R⁴⁸, R⁵¹ to R⁵⁴, R⁵⁵ to R⁵⁸, n, *, and ** are as defined in the formula (1).

28: The compound according to claim 1, wherein the compound represented by the formula (1) contains at least one deuterium atom.

29-30. (canceled)

31: An organic electroluminescent device, comprising:
a cathode, an anode, and one or more organic layers intervening between the cathode and the anode and including a light emitting layer,
wherein at least one organic layer contains the compound according to claim 1.

32: The organic electroluminescent device according to claim 31, wherein the at least one organic layer includes a hole transporting zone between the anode and the light emitting layer, and the hole transporting zone includes the compound.

33: The organic electroluminescent device according to claim 32, wherein the hole transporting zone includes a first hole transporting layer on the anode side and a second hole transporting layer on the cathode side, and at least one of the first hole transporting layer and the second hole transporting layer contains the compound.

34: The organic electroluminescent device according to claim 33, wherein the second hole transporting layer contains the compound.

35: An organic electroluminescent device, comprising:
an anode, a hole transporting zone, a light emitting layer, and a cathode in this order, wherein the hole transporting zone contains a compound A satisfying the following conditions (A) to (C):
(A) a highest occupied molecular orbital energy level HOMO is −6.00 to −5.50 eV;
(B) a triplet energy T₁ is 2.10 eV or more; and
(C) an 80% attenuation time t of photoluminescence intensity PL is 0.10 h or more, wherein PL is an intensity of a photoluminescence emission spectrum when a measurement material in which a compound to be measured is formed into a film having a film thickness of 100 nm is irradiated with ultraviolet rays of 365 nm at an irradiation intensity I₁, t is a time from the start of the irradiation with the ultraviolet rays until PL is attenuated to 80%, and I₁ is defined by the following (Equation 1):
$${I}_{1} = {I}_{0} \times ( {A}_{0} / {A}_{1} ) \qquad ( Equation 1 )$$
where,
I₀ is an irradiation intensity at the time of PL measurement of a reference material in which a compound represented by the following chemical formula is formed into a film having a film thickness of 100 nm: 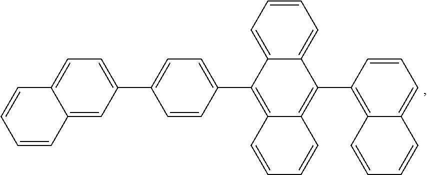 [SMILES: c1ccc2cc(-c3ccc(-c4c5ccccc5c(-c5cccc6ccccc56)c5ccccc45)cc3)ccc2c1]
A₀ is an absorptance of the reference material, and
A₁ is an absorptance of the measurement material,
each absorptance is defined by the following (Equation 2):
$$Absorptance = 1 - EXP ( - 4 \times 3.1416 \times ko \times d / w ) \qquad ( Equation 2 )$$
where,
ko is an extinction coefficient in an in-plane direction of the measurement material or the reference material on which a film of a compound to be measured is formed,
d is a film thickness of the measurement material or the reference material on which a film of the compound to be measured is formed, and
w is a wavelength of irradiation light.

36: The organic electroluminescent device according to claim 35, wherein the triplet energy T₁ in the condition (B) is 2.10 eV or more and 2.70 eV or less.

37: The organic electroluminescent device according to claim 35, wherein HOMO of the compound A is −5.95 to −5.60 eV.

38: The organic electroluminescent device according to claim 35, wherein the triplet energy T₁ of the compound A is 2.15 eV or more.

39: The organic electroluminescent device according to claim 35, wherein a lowest unoccupied molecular orbital energy level LUMO of the compound A is −1.85 to −1.30 eV.

40: The organic electroluminescent device according to claim 35, wherein the 80% attenuation time t of the compound A is 0.25 h or more.

41: The organic electroluminescent device according to claim 35, wherein the compound A is represented by the following formula (1′): 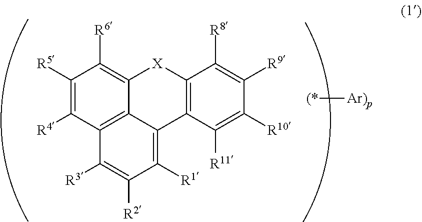
where,
X is an oxygen atom or a sulfur atom,
p is 1, 2 or 3,
R^{1′} to R^{6′} and R^{8′} to R^{11′} are each independently a hydrogen atom; a substituted or unsubstituted alkyl group having 1 to 30 carbon atoms; a substituted or unsubstituted phenyl group, a substituted or unsubstituted naphthyl group, a substituted or unsubstituted biphenyl group, a substituted or unsubstituted phenanthryl group, a substituted or unsubstituted fluorenyl group, or a substituted or unsubstituted triphenylenyl group, provided that, when p is 1, one of R^{1′} to R^{6′} and R^{8′} to R^{11′} is a single bond bonded to * or a group bonded to *; when p is 2, two of R^{1′} to R^{6′} and R^{8′} to R^{11′} are a single bond bonded to * or a group bonded to *; and when p is 3, three of R^{1′} to R^{6′} and R^{8′} to R^{11′} are a single bond bonded to * or a group bonded to *,
a pair of groups adjacent to each other among R^{1′} to R^{6′} and R^{8′} to R^{11′} which are not a hydrogen atom and which are not a single bond are not bonded to each other and do not form a ring,
Ar is,
a substituted or unsubstituted phenyl group, a substituted or unsubstituted naphthyl group, a substituted or unsubstituted biphenyl group, a substituted or unsubstituted phenanthryl group, a substituted or unsubstituted fluorenyl group, or a substituted or unsubstituted triphenylenyl group, or a substituted or unsubstituted heteroaryl group having 5 to 30 ring atoms, which is directly bonded to any of R^{1′} to R^{6′} and R^{8′} to R^{11′}; or
a substituted or unsubstituted phenyl group, a substituted or unsubstituted naphthyl group, a substituted or unsubstituted biphenyl group, a substituted or unsubstituted phenanthryl group, a substituted or unsubstituted fluorenyl group, or a substituted or unsubstituted triphenylenyl group, or a substituted or unsubstituted heteroaryl group having 5 to 30 ring atoms, bonded to a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a group formed by combining a plurality of groups of these divalent groups, which is bonded to any of R^{1′} to R^{6′} and R^{8′} to R^{11′}, and
when p is 2 or 3, a plurality of *—Ar's are the same as or different from each other.

42: The organic electroluminescent device according to claim 41, wherein *—Ar in the formula (1′) is represented by the following formula (2A′) or (2B′): 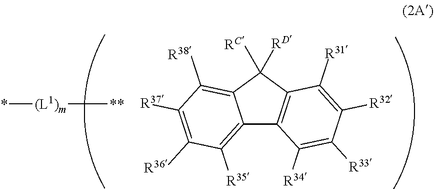
where,
L^{1′} is a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a divalent group formed by combining a plurality of groups of these groups,
m1 is 0 or 1,
R^{31′} to R^{38′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
R^{C′} and R^{D} are each independently a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R^{31′} to R^{38′}, R^{C′}, and R^{D′} is a single bond bonded to ** or a group bonded to **,
a pair of groups adjacent to each other among R^{31′} to R^{38′} which are not a single bond are bonded to each other to form a substituted or unsubstituted monocyclic ring, are bonded to each other to form a substituted or unsubstituted condensed ring, or are not bonded to each other and do not form a ring, and
R^{C′} and R^{D′} which are not a single bond are bonded to each other to form a substituted or unsubstituted monocyclic ring, are bonded to each other to form a substituted or unsubstituted condensed ring, or are not bonded to each other and do not form a ring, 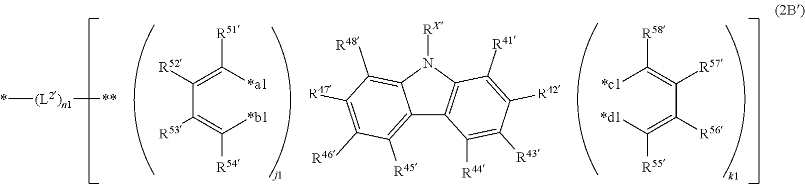
where
L^{2′} is a substituted or unsubstituted phenylene group, a substituted or unsubstituted naphthylene group, a substituted or unsubstituted biphenylene group, a substituted or unsubstituted phenanthrylene group, a substituted or unsubstituted fluorenylene group, a substituted or unsubstituted triphenylenylene group, or a divalent group formed by combining a plurality of groups of these groups,
n1 is 0 or 1,
R^{41′} to R^{48′}, R^{51′} to R^{54′}, and R^{55′} to R^{58′} are each independently a hydrogen atom, a substituted or unsubstituted alkyl group having 1 to 6 carbon atoms, a substituted or unsubstituted aryl group having 6 to 12 ring carbon atoms, or a substituted or unsubstituted heteroaryl group having 5 to 13 ring atoms,
j1 is 0 or 1,
when j1 is 1, one of R^{45′} and R^{46′}, R^{46′} and R^{47′}, or R^{47′} and R^{48′} is a single bond bonded to *a1, and the other is a single bond bonded to *b1,
k1 is 0 or 1,
when k is 1, one of R^{41′} and R^{42′}, R^{42′} and R^{43′}, or R^{43′} and R^{44′} is a single bond bonded to *c1, and the other is a single bond bonded to *d1,
R^{X′} is a hydrogen atom, a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms,
one of R^{41′} to R^{44′} which are not bonded to *c1 and *d1, R^{45′} to R^{48′} which are not bonded to *a1 and *b1, R^{51′} to R^{54′}, R^{55′} to R^{58′}, and R^{X′} is a single bond bonded to ** or a group bonded to **, and
a pair of groups adjacent to each other among R^{41′} to R^{44′} which are not bonded to *c1 and *d1 and are not a single bond bonded to **, R^{45′} to R^{48′} which are not bonded to *a1 and *b1 and are not a single bond bonded to **, R^{51′} to R^{54′} which are not a single bond bonded to **, and R^{55′} to R^{58′} which are not a single bond bonded to ** are bonded to each other to form a substituted or unsubstituted monocyclic ring, are bonded to each other to form a substituted or unsubstituted condensed ring, or are not bonded to each other and do not form a ring.

43-50. (canceled)

51: The organic electroluminescent device according to claim 35, wherein the compound A contains at least one deuterium atom.

52: The organic electroluminescent device according to claim 35, wherein the hole transporting zone includes a hole injecting layer located on the anode side and a hole transporting layer located on the cathode side.

53: The organic electroluminescent device according to claim 52, wherein the hole transporting zone includes a first hole transporting layer on the anode side and a second hole transporting layer on the cathode side, and at least one of the first hole transporting layer and the second hole transporting layer contain the compound A.

54: The organic electroluminescent device according to claim 53, wherein the second hole transporting layer contains the compound A.

55-56. (canceled)

57: The organic electroluminescent device according to claim 31, further comprising:
an electron transporting zone between the light emitting layer and the cathode,
wherein the electron transporting zone includes a first electron transporting layer on the light emitting layer side and a second electron transporting layer on the cathode side.

58: The organic electroluminescent device according to claim 31, further comprising:
an electron transporting zone between the light emitting layer and the cathode,
wherein the electron transporting zone includes an electron transporting layer located on the light emitting layer side and an electron injecting layer located on the cathode side.

59: The organic electroluminescent device according to claim 31, wherein the light emitting layer contains a layer containing a light emitting compound exhibiting fluorescence emission with a main peak wavelength of 500 nm or less.

60: The organic electroluminescent device according to claim 31, wherein the light emitting layer is a single layer.

61: The organic electroluminescent device according to claim 31, comprising one or more light emitting units each including an organic layer at least one layer of which is the light emitting layer,
wherein the light emitting layer in one of the light emitting units consists of only one host material.

62. (canceled)
